(19) United States
(12) Patent Application Publication
(10) Pub. No.: US 2026/0173647 A1
(43) Pub. Date: Jun. 18, 2026

(54) ORGANIC ELECTROLUMINESCENT COMPOUND AND ORGANIC ELECTROLUMINESCENT DEVICE COMPRISING THE SAME

(71) Applicant: DuPont Specialty Materials Korea Ltd., Chungcheongnam-do (KR)

(72) Inventors: Jin-Man KIM, Gyeonggi-do (KR); Hyun KIM, Gyeonggi-do (KR); Hyun-Woo KANG, Gyeonggi-do (KR); Hong-Se OH, Gyeonggi-do (KR); Hyo-Soon PARK, Gyeonggi-do (KR); Soo-Yong LEE, Gyeonggi-do (KR); Han-Kyu YOO, Gyeonggi-do (KR); Mi-Ja LEE, Gyeonggi-do (KR); Hyo-Jung LEE, Gyeonggi-do (KR)

(21) Appl. No.: 19/410,028

(22) Filed: Dec. 5, 2025

(30) Foreign Application Priority Data
Dec. 18, 2024 (KR) 10-2024-0189786
Nov. 12, 2025 (KR) 10-2025-0170571

(51) Int. Cl. H10K 50/155; C07B 59/00; C07C 211/55; C09K 11/06; H10K 50/19; H10K 85/30; H10K 85/40; H10K 85/60

(52) U.S. Cl. CPC H10K 50/155; C07B 59/004; C07C 211/55; C09K 11/06; H10K 50/19; H10K 85/342; H10K 85/40; H10K 85/622; H10K 85/626; H10K 85/633; H10K 85/636; H10K 85/654; H10K 85/656; H10K 85/6572; H10K 85/6574; H10K 85/6576; C07B 2200/05; C09K 2211/1475

(57) ABSTRACT

The present disclosure relates to an organic electroluminescent compound represented by Formula 1, and an organic electroluminescent material and an organic electroluminescent device comprising the same. By comprising the compound according to the present disclosure, it is possible to provide an organic electroluminescent device having a lower driving voltage, higher luminous efficiency and/or improved lifetime characteristics compared to the prior art.

### TECHNICAL FIELD

The present disclosure relates to an organic electroluminescent compound and an organic electroluminescent device comprising the same.

### BACKGROUND ART

The TPD/Alq3 bilayer small-molecule organic electroluminescent device (OLED) with green emission, which is constituted with a light-emitting layer and a charge transport layer, was first developed by Tang et al. of Eastman Kodak in 1987. Thereafter, studies on organic electroluminescent devices have proceeded rapidly, and OLEDs have since been commercialized. At present, OLEDs primarily use phosphorescent materials having excellent luminous efficiency in panel implementation. For long-term use of displays and high resolution, OLEDs with high luminous efficiency are required.

Chinese Patent Application Laid-Open Nos. 117142962, 114773286, and 116283865 disclose fluorene derivatives or compounds comprising amino groups, but do not specifically disclose the particular compounds claimed in the present disclosure. In addition, there is a continuous need to develop light-emitting materials that exhibit improved performance compared to previously disclosed compounds, such as enhanced driving voltage, luminous efficiency, and/or lifetime characteristics.

### DISCLOSURE OF INVENTION

### Technical Problem

The objective of the present disclosure is to provide an organic electroluminescent compound having a novel structure that is suitable for application in organic electroluminescent devices. Another objective of the present disclosure is to provide an organic electroluminescent material and an organic electroluminescent device exhibiting high luminous efficiency and/or long lifetime characteristics.

### Solution to Problem

As a result of intensive studies to solve the technical problems, the present inventors found that the above objective can be achieved by an organic electroluminescent compound represented by the following Formula 1, and an organic electroluminescent material or an organic electroluminescent device comprising the same.

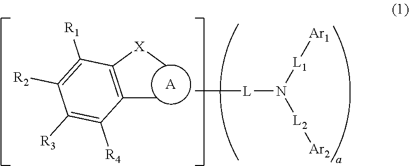

In Formula 1,
X represents —O—, —S—, —Se—, —Ge— or —(CR₉R₁₀)ₙ—; ring A represents a substituted or unsubstituted benzene ring, or a substituted or unsubstituted naphthalene ring, and two adjacent carbon atoms in ring A are each bonded to the * positions in Formula 1-A to form an aliphatic ring.

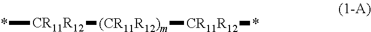

R₁ to R₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L-N(L₁-Ar₁)(L₂-Ar₂);
R₉ and R₁₀ each independently represent a substituted or unsubstituted (C1-C30)alkyl, or a substituted or unsubstituted (C6-C30)aryl, or R₉ and R₁₀ may be linked to each other to form a ring(s);
R₁₁ and R₁₂ each independently represent hydrogen, deuterium, a halogen, a substituted or unsubstituted (C1-C30)alkyl, or a substituted or unsubstituted (C6-C30)aryl, and each R₁ or each R₁₂ may be the same as or different from each other;
L, L₁, and L₂ each independently represent a single bond, a substituted or unsubstituted (C6-C30)arylene, a substituted or unsubstituted (3- to 30-membered)heteroarylene, or a substituted or unsubstituted (C3-C30)cycloalkylene;
L is linked to at least one selected from the group consisting of a carbon atom bonded to any one of R₁ to R₄, and a carbon atom of the benzene ring or the naphthalene ring of the ring A;
Ar₁ and Ar₂ each independently represent a substituted or unsubstituted (C6-C30)aryl, a substituted or unsubstituted (3- to 30-membered)heteroaryl, or a substituted or unsubstituted (C3-C30)cycloalkyl;
n represents an integer of 1 or 2, m represents an integer of 1 to 3, a represents an integer of 1 or 2; and
hydrogen atoms in the compound represented by Formula 1 may be substituted with deuterium;
with the proviso that if ring A is a substituted or unsubstituted benzene ring, L₁, L₂, Ar₁, and Ar₂ do not comprise a heteroaryl or an aryl fused with a cycloalkyl.

In addition, the present inventors found that the above objective can be achieved by an organic electroluminescent device comprising a first electrode; a second electrode facing the first electrode; a light-emitting layer between the first electrode and the second electrode, and a hole transport zone between the first electrode and the light-emitting layer, wherein the hole transport zone comprises the compound represented by Formula 1, wherein the light-emitting layer comprises a compound represented by the following Formula 6 and a compound represented by the following Formula 7.

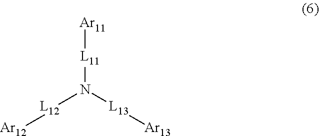

In Formula 6,
L₁₁ to L₁₃ each independently represent a single bond, a substituted or unsubstituted (C6-C30)arylene, and a substituted or unsubstituted (3- to 30-membered)heteroarylene;
Ar₁₁ represents a substituted or unsubstituted (3- to 30-membered)heteroaryl;
Ar₁₂ and Ar₁₃ each independently represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C2-C30)alkenylamino, a substituted or unsubstituted (C1-C30)alkyl(C2-C30)alkenylamino, a substituted or unsubstituted mono- or di(C6-C30)arylamino, a substituted or unsubstituted (C1-C30)alkyl(C6-C30)arylamino, a substituted or unsubstituted mono- or di(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino.

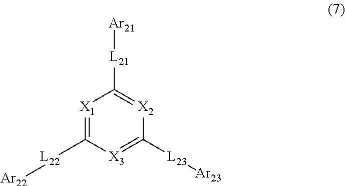

In Formula 7,
X₁ to X₃ each independently represent N or CR₂₁; with the proviso that at least one of X₁ to X₃ is N;
each R₂₁ independently represents hydrogen or deuterium;
L₂₁ to L₂₃ each independently represent a single bond, a substituted or unsubstituted (C6-C30)arylene, or a substituted or unsubstituted (3- to 30-membered)heteroarylene; and
Ar₂₁ to Ar₂₃ each independently represent a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl;
with the proviso that at least one of Ar₂₁ to Ar₂₃ is a substituted or unsubstituted (3- to 30-membered)heteroaryl.

### Advantageous Effects of Invention

An organic electroluminescent compound according to the present disclosure exhibits performance suitable for use in organic electroluminescent devices. In addition, by comprising the compound according to the present disclosure as an organic electroluminescent material, it is possible to provide an organic electroluminescent device exhibiting higher luminous efficiency and/or improved lifetime characteristics compared to conventional organic electroluminescent devices, and to manufacture a display device or lighting device using the same.

### MODE FOR INVENTION

Hereinafter, the present disclosure will be described in detail. However, the following description is intended to explain the present disclosure, and is not meant in any way to restrict the scope of the present disclosure.

The “organic electroluminescent compound” in the present disclosure is a compound that may be used in an organic electroluminescent device, and may be comprised in any layer constituting an organic electroluminescent device, as necessary.

The “organic electroluminescent material” in the present disclosure is a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a charge-generating material, an N-type charge-generating material, a P-type charge-generating material, a light-emitting auxiliary material, an electron-blocking material, a light-emitting material (including a host material and a dopant material), an electron buffer material, a hole-blocking material, an electron transport material, an electron injection material, etc.

Herein, the term “(C1-C30)alkyl” is meant to refer to a linear or branched alkyl having 1 to 30 carbon atoms constituting a chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C3-C30)cycloalkyl” is meant to refer to a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, norbornyl, adamantly, etc. The term “(3- to 7-membered)heterocycloalkyl” in the present disclosure is meant to refer to a saturated or partially unsaturated monocyclic or polycyclic ring-shaped hydrocarbon substituent having 3 to 7, preferably 5 to 7 ring backbone atoms, and including at least one heteroatom selected from the group consisting of B, N, O, S, Si, P, and Se. The above heterocycloalkyl may include tetrahydrofuran, pyrrolidine, thiolane, tetrahydropyran, etc.

The “(C6-C30)aryl” or “(C6-C30)arylene” in the present disclosure refers to a monocyclic or fused ring radical derived from an aromatic hydrocarbon having 6 to 30 ring backbone carbon atoms, and may be partially saturated. The number of the ring backbone carbon atoms is preferably 6 to 20, more preferably 6 to 15. The above aryl may comprise a spiro structure. The above aryl may include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. Specifically, the aryl may include o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The “(3- to 30-membered)heteroaryl” or “(3- to 30-membered)heteroarylene” in the present disclosure refers to an aryl group or arylene group having 3 to 30 ring backbone atoms and including at least one heteroatom(s) selected from the group consisting of B, N, O, S, Si, P, and Se. Herein, the number of ring backbone atoms is preferably 3 to 30, and more preferably 5 to 20. The number of heteroatoms is preferably 1 to 4. The above heteroaryl or heteroarylene may be a monocyclic ring or a fused ring condensed with at least one benzene ring, and may be partially saturated. In addition, the above heteroaryl or heteroarylene may be one formed by linking at least one heteroaryl or aryl group to a heteroaryl group via a single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzisothiazolyl, benzisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolyl, thiochromenoquinazolyl, dimethylbenzopyrimidinyl, indolocarbazolyl, indenocarbazolyl, etc. More specifically, the heteroaryl may include 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl,3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc.

Additionally, “heteroaryl(ene)” can be classified into a heteroaryl(ene) with electronic properties and a heteroaryl(ene) with hole properties. A heteroaryl(ene) with electronic properties is a substituent that is relatively rich in electrons in the parent nucleus, for example, a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene) with hole properties is a substituent that is relatively electron-deficient in the parent nucleus, for example, a substituted or unsubstituted carbazolyl, a substituted or unsubstituted dibenzofuranyl, a substituted or unsubstituted dibenzothiophenyl, etc.

Herein, “a fused ring group of a (C3-C30) aliphatic ring(s) and a (C6-C30) aromatic ring(s)” is meant to be a functional group of a ring in which at least one aliphatic ring having 3 to 30 ring backbone carbon atoms, preferably 3 to 25 ring backbone carbon atoms, and more preferably 3 to 18 ring backbone carbon atoms, is fused with at least one aromatic ring having 6 to 30 ring backbone carbon atoms, preferably 6 to 25 ring backbone carbon atoms, and more preferably 6 to 18 ring backbone carbon atoms. Specific examples of the fused ring group include a fused ring group of one or more benzene and one or more cyclohexane, or a fused ring group of one or more naphthalene and one or more cyclopentane, etc. Herein, the carbon atom of the fused ring group of a (C3-C30) aliphatic ring(s) and a (C6-C30) aromatic ring(s) may be replaced with one or more heteroatoms selected from B, N, O, S, Si, P, and Se. Herein, “halogen” includes F, Cl, Br, and I.

In addition, “ortho-” (“o-”), “meta-” (“m-”), and “para” (“p-”) are prefixes which each represent the relative positions of substituents. The prefix “ortho-” indicates that two substituents are adjacent to each other, and for example, when two substituents in a benzene derivative occupy positions 1 and 2, this is called an “ortho-” configuration. The prefix “meta-” indicates that two substituents are at positions 1 and 3, and for example, when two substituents in a benzene derivative occupy positions 1 and 3, this is called a “meta-” configuration. The prefix “para-” indicates that two substituents are at positions 1 and 4, and for example, when two substituents in a benzene derivative occupy positions 1 and 4, this is called a “para-” configuration.

Herein, “a ring formed by being linked to an adjacent substituent(s)” means that at least two adjacent substituents are linked to or fused with each other to form a substituted or unsubstituted, mono- or polycyclic, (3- to 30-membered) alicyclic or aromatic ring, or a combination thereof. Preferably, the ring may be a substituted or unsubstituted, mono- or polycyclic, (5- to 25-membered) alicyclic or aromatic ring, or a combination thereof. In addition, the ring may contain at least one heteroatom selected from B, N, O, S, Si, P, and Se. According to one embodiment of the present disclosure, the number of ring backbone carbon atoms is 5 to 20, and according to another embodiment of the present disclosure, the number of ring backbone carbon atoms is 5 to 15. For example, the fused ring may have a spiro structure and may be in the form of, for instance, a substituted or unsubstituted fluorene ring, a substituted or unsubstituted benzofluorene ring, etc.

Herein, “substituted” in the expression “substituted or unsubstituted” means that a hydrogen atom in a certain functional group is replaced with another atom or another functional group, i.e., a substituent, and this also includes substitution by a group in which two or more of the substituents are connected. Unless otherwise specified, the substituent may replace hydrogen at a position where the substituent can be substituted without limitation, and when two or more hydrogen atoms in a certain functional group are each replaced with a substituent, each substituent may be the same as or different from each other. The maximum number of substituents that can be substituted for a certain functional group may be the total number of valences that can be substituted for each atom forming the functional group. Herein, the substituted alkyl, the substituted aryl(ene), the substituted heteroaryl(ene), the substituted cycloalkyl(ene), the substituted heterocycloalkyl(ene), the substituted alkoxy, the substituted trialkylsilyl, the substituted dialkylarylsilyl, the substituted alkyldiarylsilyl, the substituted triarylsilyl, and the substituted fused ring group of an aliphatic ring(s) and an aromatic ring(s), the substituted benzene ring, the substituted naphthalene ring, the substituted mono- or dialkylamino, the substituted mono- or dialkenylamino, the substituted alkylalkenylamino, the substituted mono- or diarylamino, the substituted alkylarylamino, the substituted alkylheteroarylamino, the substituted alkenylarylamino, the substituted alkenylheteroarylamino, the substituted mono- or diheteroarylamino, and the substituted arylheteroarylamino each independently may be substituted with at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (3- to 30-membered)heteroaryl, a (C6-C30)aryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, a fused ring group of a (C3-C30) aliphatic ring(s) and a (C6-C30) aromatic ring(s), an amino, a mono- or di(C1-C30)alkylamino, a substituted or unsubstituted mono- or di(C6-C30)arylamino, a substituted or unsubstituted (C1-C30)alkyl(C6-C30)arylamino, a substituted or unsubstituted mono- or di(3- to 30-membered)heteroarylamino, a substituted or unsubstituted (C1-C30)alkyl(3- to 30-membered)heteroarylamino, a substituted or unsubstituted (C6-C30)aryl(3- to 30-membered)heteroarylamino, a (C1-C30)alkylcarbonyl, a (C1-C30)alkoxycarbonyl, a (C6-C30)arylcarbonyl, a (C6-C30)arylphosphinyl, a di(C6-C30)arylboronyl, a di(C1-C30)alkylboronyl, a (C1-C30)alkyl(C6-C30)arylboronyl, a (C6-C30)aryl(C1-C30)alkyl, a (C1-C30)alkyl(C6-C30)aryl, and a combination thereof. According to one embodiment of the present disclosure, the group consists of deuterium, a halogen, a (C1-C20)alkyl, a (C3-C20)cycloalkyl, a (C6-C25)aryl, a (3- to 25-membered)heteroaryl, a mono- or di(C6-C30)arylamino and a combination thereof.

According to another embodiment of the present disclosure, the group consists of deuterium, a halogen, a (C1-C10)alkyl, a (C6-C10)cycloalkyl, a (C6-C18)aryl, a (3- to 25-membered)heteroaryl, a mono- or di(C6-C20)arylamino and a combination thereof. For example, the group may constist of deuterium, a methyl, an ethyl, a tert-butyl, a phenyl, a naphthyl, a biphenyl, a phenanthrenyl, a dibenzofuranyl, a dibenzothiophenyl, a pyridyl, a triphenylenyl, a diphenylamino, a carbazolyl, a cyclohexyl, a norbornyl, or an adamantyl etc., and these may be further substituted with deuterium.

In the present disclosure, if a substituent is not indicated in the chemical formula or compound structure, it may mean that all possible positions for the substituent are hydrogen or deuterium. That is, in the case of deuterium, it is an isotope of hydrogen, and some hydrogen atoms may be the isotope deuterium, and in this case, the content of deuterium may be 0% to 100%. In the present disclosure, in cases where a substituent is not indicated in the chemical formula or compound structure, if the substituent is not explicitly excluded, such as 0% deuterium, 100% hydrogen, and all substituents are hydrogen, hydrogen and deuterium may be used intermixed in a compound. Deuterium is an isotope of hydrogen and an element with a deuteron consisting of one proton and one neutron, as its nucleus. It can be represented as hydrogen-2, whose element symbol can also be written as D or ²H. Isotopes are atoms with the same atomic number (Z) but different mass numbers (A), and can also be interpreted as elements with the same number of protons but different numbers of neutrons.

In the present disclosure, “a combination thereof” refers to a combination of one or more elements from the corresponding list to form a known or chemically stable arrangement that can be envisioned from the corresponding list by a person skilled in the art. For example, alkyl and deuterium can be combined to form a partially or fully deuterated alkyl group, a halogen and alkyl can be combined to form a halogenated alkyl substituent, and a halogen, alkyl, and aryl can be combined to form a halogenated arylalkyl. For example, a preferred combination of substituents includes up to 50 atoms that are not hydrogen or deuterium, or up to 40 atoms that are not hydrogen or deuterium, or up to 30 atoms that are not hydrogen or deuterium, or in many cases, a preferred combination of substituents may comprise up to 20 atoms that are not hydrogen or deuterium.

In the formulas of the present disclosure, when there are multiple substituents represented by the same symbol, each substituent represented by the same symbol may be the same as or different from each other.

The compound represented by Formula 1 is described in more detail as follows.

In Formula 1, X represents —O—, —S—, —Se—, —Ge— or —(CR₉R₁₀)ₙ—. According to one embodiment of the present disclosure, X represents —(CR₉R₁₀)ₙ—.

In Formula 1, R₉ and R₁₀ each independently represent a substituted or unsubstituted (C1-C30)alkyl, or a substituted or unsubstituted (C6-C30)aryl, or R₉ and R₁₀ may be linked to each other to form a ring(s). According to one embodiment of the present disclosure, R₉ and R₁₀ each independently represent a substituted or unsubstituted (C1-C20)alkyl, or a substituted or unsubstituted (C6-C20)aryl, or R₉ and R₁₀ may be linked to each other to form a spiro ring(s). According to another embodiment of the present disclosure, R₉ and R₁₀ each independently represent a substituted or unsubstituted (C1-C10)alkyl, or a substituted or unsubstituted (C6-C18)aryl. For example, R₉ and R₁₀ may each independently be a methyl, an ethyl, or a phenyl, and these may be substituted with deuterium. R₉ and R₁₀ may be the same as or different from each other. According to one embodiment of the present disclosure, R₉ and R₁₀ may be the same as.

In Formula 1, n represents an integer of 1 or 2. According to one embodiment of the present disclosure, n is 1.

In Formula 1, ring A represents a substituted or unsubstituted benzene ring, or a substituted or unsubstituted naphthalene ring, and two adjacent carbon atoms in ring A are each bonded to the * positions in Formula 1-A to form an aliphatic ring. According to one embodiment of the present disclosure, the benzene ring or the naphthalene ring may be substituted with one or more of deuterium, a phenyl, and -L-N(L₁-Ar₁)(L₂-Ar₂), and the two adjacent carbon atoms in the benzene ring or the naphthalene ring may each be bonded to the * positions in Formula 1-A to form a cyclohexane, cyclopentane, or cycloheptane ring.

In Formula 1, R₁ to R₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L-N(L₁-Ar₁)(L₂-Ar₂). According to one embodiment of the present disclosure, R₁ to R₄ each independently represent hydrogen, deuterium, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or -L-N(L₁-Ar₁)(L₂-Ar₂). According to another embodiment of the present disclosure, R₁ to R₄ each independently represent hydrogen, deuterium, a substituted or unsubstituted (C6-C30)aryl, or -L-N(L₁-Ar₁)(L₂-Ar₂). For example, R₁ to R₄ each independently represent hydrogen, deuterium, or -L-N(L₁-Ar₁)(L₂-Ar₂).

In Formula 1-A, R₁₁ and R₁₂ each independently represent hydrogen, deuterium, a halogen, a substituted or unsubstituted (C1-C30)alkyl, or a substituted or unsubstituted (C6-C30)aryl, and each R₁ or each R₁₂ may be the same as or different from each other. According to one embodiment of the present disclosure, R₁₁ and R₁₂ each independently represent hydrogen, or deuterium.

In Formula 1-A, m represents an integer of 1 to 3.

In Formula 1, L, L₁, and L₂ each independently represent a single bond, a substituted or unsubstituted (C6-C30)arylene, a substituted or unsubstituted (3- to 30-membered)heteroarylene, or a substituted or unsubstituted (C3-C30)cycloalkylene. According to one embodiment of the present disclosure, L, L₁, and L₂ each independently represent a single bond, or a substituted or unsubstituted (C6-C25)arylene. According to another embodiment of the present disclosure, L, L₁ and L₂ each independently represent a single bond, or a substituted or unsubstituted (C6-C18)arylene. Herein, arylene, heteroarylene, or cycloalkylene may be substituted with one or more of deuterium, (C1-C30)alkyl, and (C6-C30)aryl, and these may be further substituted with deuterium. For example, L may be a single bond, L₁ and L₂ may each independently be a single bond, a phenylene unsubstituted or substituted with a phenyl, a biphenyl, a dimethylfluorenylene, etc., and these may be further substituted with deuterium.

In Formula 1, L is linked to at least one selected from the group consisting of a carbon atom bonded to any one of R₁ to R₄, and a carbon atom of the benzene ring or the naphthalene ring of the ring A. According to one embodiment of the present disclosure, L is linked to at least one selected from the group consisting of a carbon atom bonded to any one of R₁ to R₄, and/or the benzene ring or the naphthalene ring of the ring A. For example, L may be linked to a carbon atom bonded to R₂, R₃, or R₄ and/or the benzene ring of the ring A.

In Formula 1, Ar₁ and Ar₂ each independently represent a substituted or unsubstituted (C6-C30)aryl, a substituted or unsubstituted (3- to 30-membered)heteroaryl, or a substituted or unsubstituted (C3-C30)cycloalkyl. According to one embodiment of the present disclosure, An and Ar₂ each independently represent a substituted or unsubstituted (C6-C25)aryl, or a substituted or unsubstituted (C6-C25)cycloalkyl. According to another embodiment of the present disclosure, Ar₁ and Ar₂ each independently represent a substituted or unsubstituted (C6-C25)aryl, or a substituted or unsubstituted (C6-C10)cycloalkyl. Herein, aryl, heteroaryl, or cycloalkyl may be substituted with at least one selected from the group consisting of deuterium, a (C1-C30)alkyl, a (C6-C30)aryl, and a (C3-C30)cycloalkyl. For example, Ar₁ and Ar₂ may each independently be a substituted or unsubstituted phenyl, a naphthyl, a biphenyl, a terphenyl, a quaterphenyl, a dimethylfluorenyl, a diphenylfluorenyl, a cyclohexyl, a norbornyl, an adamantyl, etc., and these may be further substituted with deuterium. The phenyl may be substituted with one or more of deuterium, a methyl, a tert-butyl, a norbornyl, an adamantyl, and a cyclohexyl. According to one embodiment of the present disclosure, at least one of Ar₁ and Ar₂ comprises a (C6-C30)aryl substituted with a (C1-C30)alkyl, or a (C3-C30)cycloalkyl, and preferably, at least one of Ar₁ and Ar₂ may comprise a (C6-C30)aryl substituted with a methyl, a tert-butyl, a norbornyl, an adamantyl, or a cyclohexyl.

In Formula 1, a represents an integer of 1 or 2. The hydrogen atoms in the compound represented by Formula 1 may be substituted with deuterium.

In Formula 1, if ring A is a substituted or unsubstituted benzene ring, L₁, L₂, Ar₁, and Ar₂ do not comprise a heteroaryl or an aryl fused with a cycloalkyl. According to one embodiment of the present disclosure, if ring A is a substituted or unsubstituted benzene ring, L₁, L₂, Ar₁, and Ar₂ do not comprise a (3- to 30-membered)heteroaryl or a (C6-C30)aryl fused with a (C3-C30)cycloalkyl. For example, an aryl fused with the cycloalkyl may be

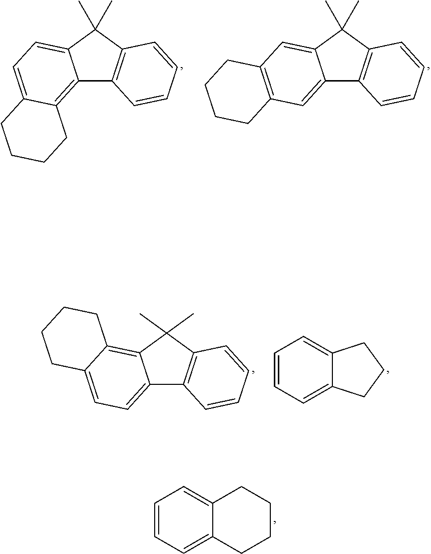

Formula 1 may be represented by any one of the following Formulas 2-1 to 2-4.

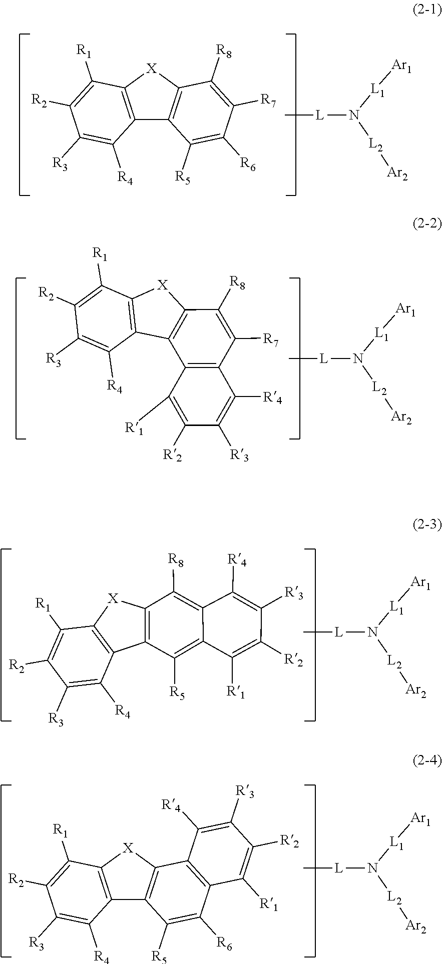

In Formulas 2-1 to 2-4, one pair selected from R₅ and R₆, R₆ and R₇, and R₇ and R₈ is each linked to * positions of Formula 1-A, or one pair selected from R′₁ and R′₂, R′₂ and R′₃, and R′₃ and R′₄ is each linked to * positions of Formula 1-A, and X, R₁ to R₄, L, L₁, L₂, Ar₁, and Ar₂ are as defined in Formula 1, and R₅ to R₈ and R′₁ to R′₄ which are not linked to Formula 1-A, are each independently the same as defined for R₁.

According to one embodiment of the present disclosure, in Formulas 2-1 to 2-4, L may be bonded to a carbon atom that is bonded to one or more of R₁ to R₈, and R′₁ to R′₄. For example, L may be bonded to a carbon atom that is bonded to one or more of R₂, R₃, R₄, and R₅.

Formula 1 may be represented by any one of the following Formulas 3-1 to 3-4.

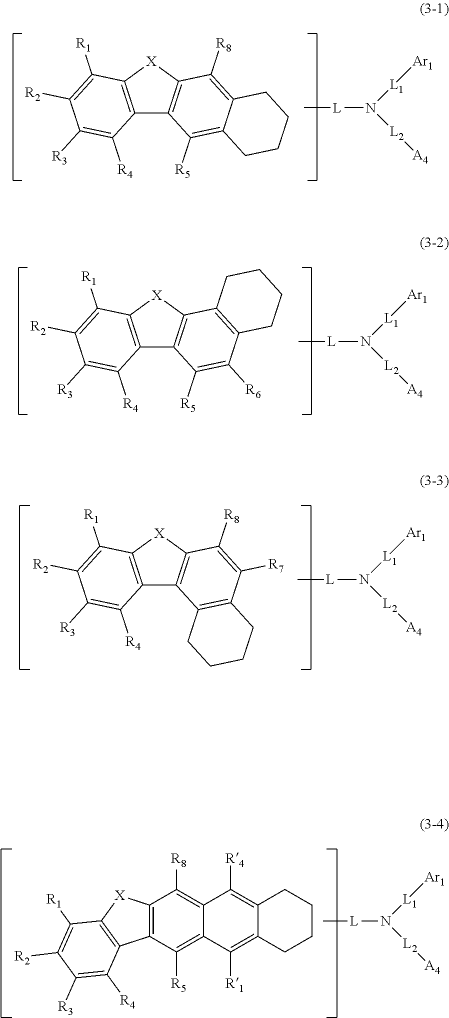

In Formulas 3-1 to 3-4, X, R₁ to R₄, L, L₁, L₂, Ar₁, and Ar₂ are as defined in Formula 1, and R₅ to R₈, R′₁ and R′₄ are each independently the same as defined for R₁.

According to one embodiment of the present disclosure, in Formulas 3-1 to 3-4, L may be bonded to a carbon atom that is bonded to one or more of R₁ to R₈, R′₁ and R′₄. For example, L may be bonded to a carbon atom that is bonded to one or more of R₂, R₃, R₄, and R₅.

Formula 1 may be represented by any one of the following Formulas 4-1 to 4-4.

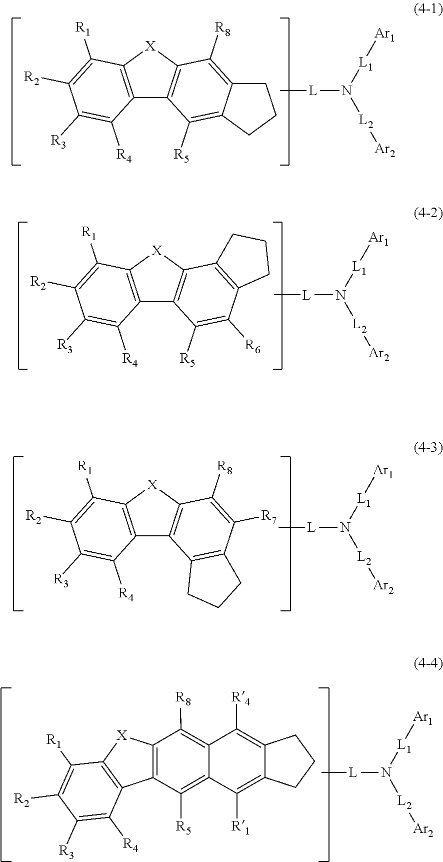

In Formulas 4-1 to 4-4, X, R₁ to R₄, L, L₁, L₂, Ar₁, and Ar₂ are as defined in Formula 1, and R₅ to R₈, R′₁ and R′₄ are each independently the same as defined for R₁.

According to one embodiment of the present disclosure, in Formulas 4-1 to 4-4, L may be bonded to a carbon atom that is bonded to one or more of R₁ to R₈, R′₁ and R′₄. For example, L may be bonded to a carbon atom that is bonded to one or more of R₂ and R₅.

Formula 1 is represented by any one of the following Formulas 5-1 to 5-4.

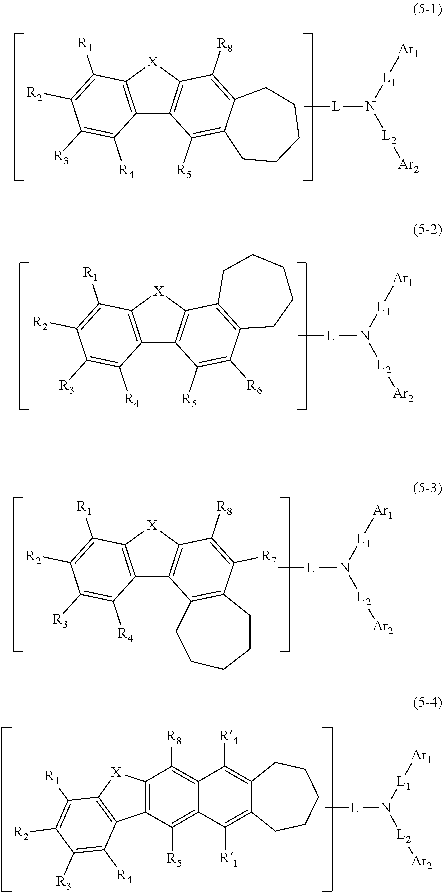

In Formulas 5-1 to 5-4, X, R₁ to R₄, L, L₁, L₂, Ar₁, and Ar₂ are as defined in Formula 1, and R₅ to R₈, R′₁ and R′₄ are each independently the same as defined for R₁.

According to one embodiment of the present disclosure, in Formulas 5-1 to 5-4, L may be bonded to a carbon atom that is bonded to one or more of R₁ to R₈, R′₁ and R′₄. For example, L may be bonded to a carbon atom that is bonded to one or more of R₂ and R₅.

The compound represented by Formula 1 is selected from the following compounds, but limited to thereto.

The organic electroluminescent compound represented by Formula 1 may be at least one selected from the following compounds, but is not limited thereto.

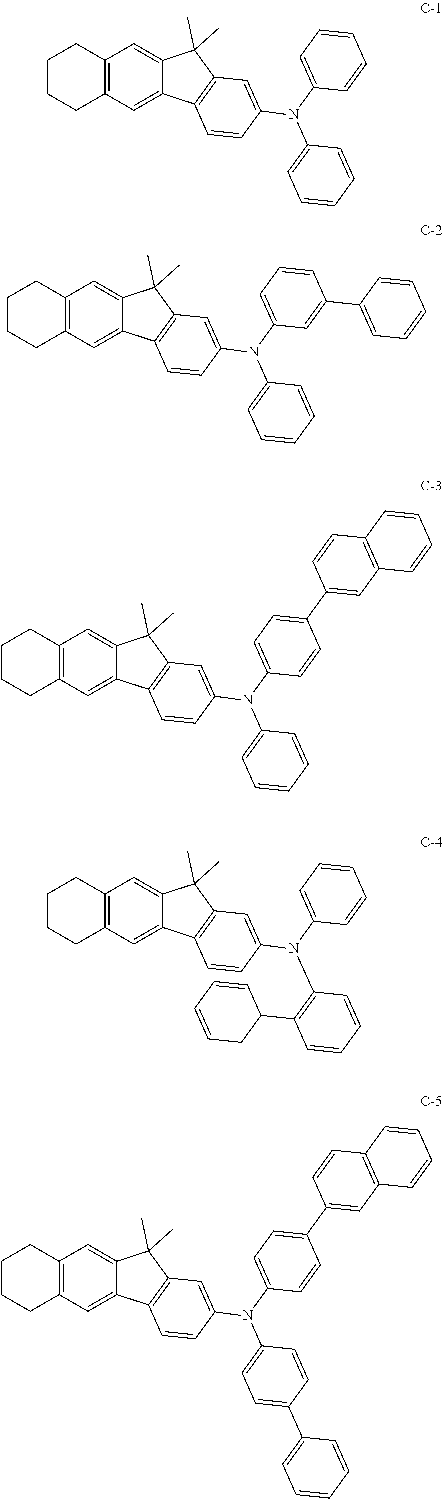 [SMILES: CC1(C)c2cc(N(c3ccccc3)c3ccccc3)ccc2-c2cc3c(cc21)CCCC3 ; CC1(C)c2cc(N(c3ccccc3)c3cccc(-c4ccccc4)c3)ccc2-c2cc3c(cc21)CCCC3 ; CC1(C)c2cc(N(c3ccccc3)c3ccc(-c4ccc5ccccc5c4)cc3)ccc2-c2cc3c(cc21)CCCC3 ; CC1(C)c2cc(N(c3ccccc3)c3ccccc3C3C=CC=CC3)ccc2-c2cc3c(cc21)CCCC3 ; CC1(C)c2cc(N(c3ccc(-c4ccccc4)cc3)c3ccc(-c4ccc5ccccc5c4)cc3)ccc2-c2cc3c(cc21)CCCC3]

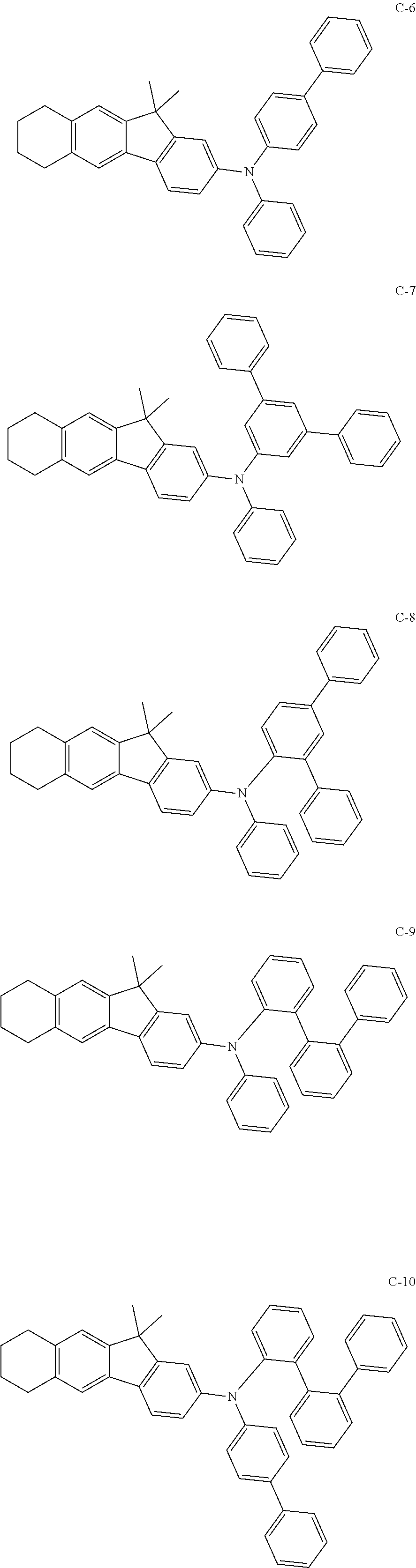 [SMILES: CC1(C)c2cc(N(c3ccccc3)c3ccc(-c4ccccc4)cc3)ccc2-c2cc3c(cc21)CCCC3 ; CC1(C)c2cc(N(c3ccccc3)c3cc(-c4ccccc4)cc(-c4ccccc4)c3)ccc2-c2cc3c(cc21)CCCC3 ; CC1(C)c2cc(N(c3ccccc3)c3ccc(-c4ccccc4)cc3-c3ccccc3)ccc2-c2cc3c(cc21)CCCC3 ; CC1(C)c2cc(N(c3ccccc3)c3ccccc3-c3ccccc3-c3ccccc3)ccc2-c2cc3c(cc21)CCCC3 ; CC1(C)c2cc(N(c3ccc(-c4ccccc4)cc3)c3ccccc3-c3ccccc3-c3ccccc3)ccc2-c2cc3c(cc21)CCCC3]

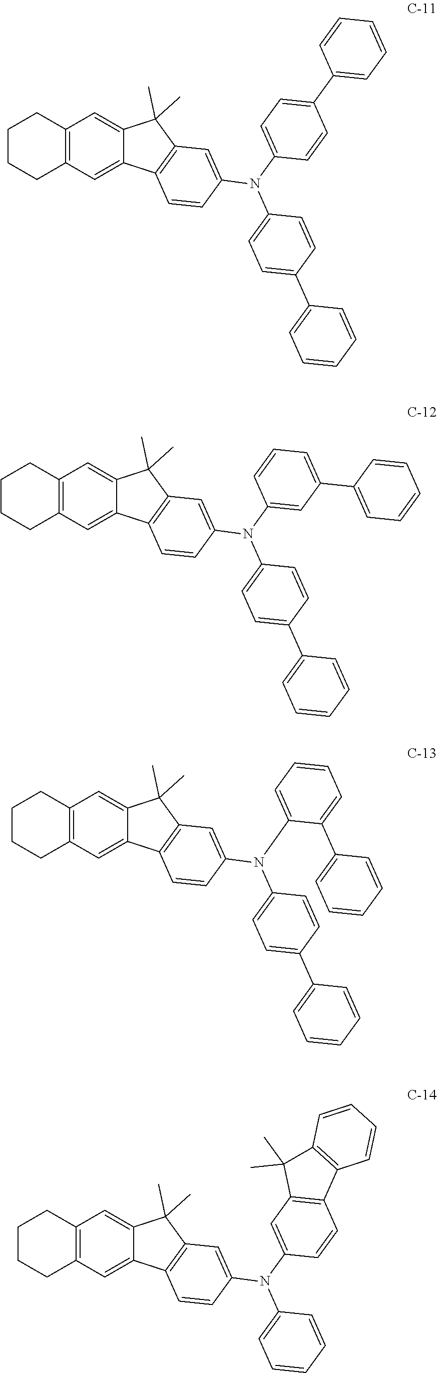 [SMILES: CC1(C)c2cc(N(c3ccc(-c4ccccc4)cc3)c3ccc(-c4ccccc4)cc3)ccc2-c2cc3c(cc21)CCCC3 ; CC1(C)c2cc(N(c3ccc(-c4ccccc4)cc3)c3cccc(-c4ccccc4)c3)ccc2-c2cc3c(cc21)CCCC3 ; CC1(C)c2cc(N(c3ccc(-c4ccccc4)cc3)c3ccccc3-c3ccccc3)ccc2-c2cc3c(cc21)CCCC3 ; CC1(C)c2ccccc2-c2ccc(N(c3ccccc3)c3ccc4c(c3)C(C)(C)c3cc5c(cc3-4)CCCC5)cc21]

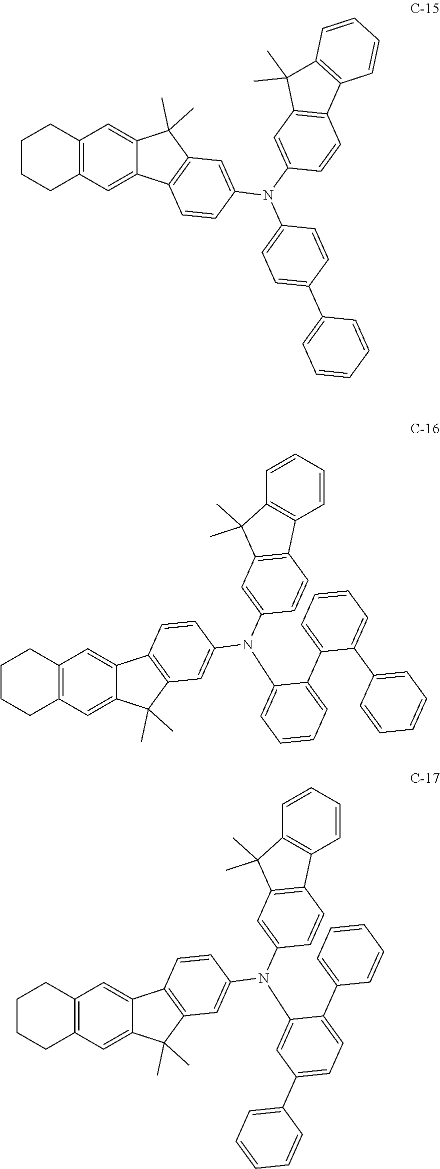 [SMILES: CC1(C)c2ccccc2-c2ccc(N(c3ccc(-c4ccccc4)cc3)c3ccc4c(c3)C(C)(C)c3cc5c(cc3-4)CCCC5)cc21 ; CC1(C)c2ccccc2-c2ccc(N(c3ccc4c(c3)C(C)(C)c3cc5c(cc3-4)CCCC5)c3ccccc3-c3ccccc3-c3ccccc3)cc21 ; CC1(C)c2ccccc2-c2ccc(N(c3ccc4c(c3)C(C)(C)c3cc5c(cc3-4)CCCC5)c3cc(-c4ccccc4)ccc3-c3ccccc3)cc21]

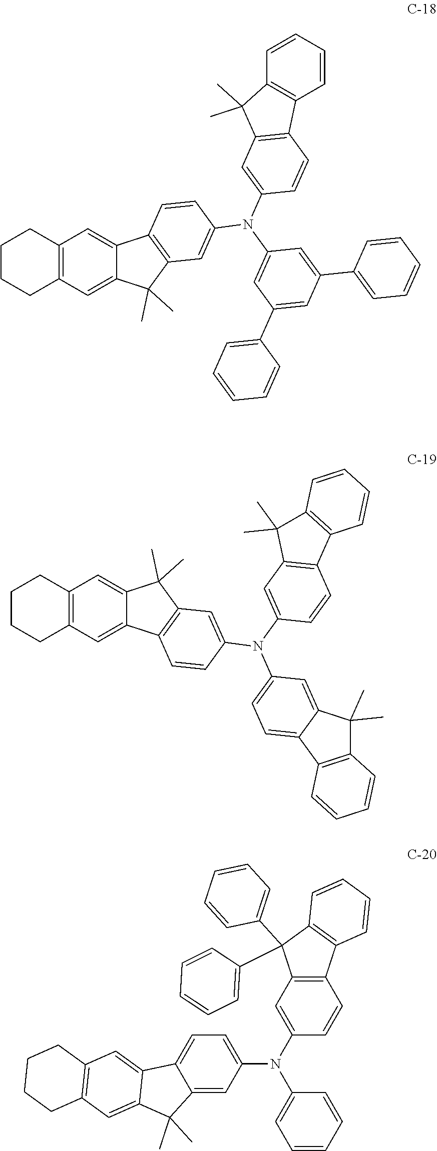 [SMILES: CC1(C)c2ccccc2-c2ccc(N(c3cc(-c4ccccc4)cc(-c4ccccc4)c3)c3ccc4c(c3)C(C)(C)c3cc5c(cc3-4)CCCC5)cc21 ; CC1(C)c2ccccc2-c2ccc(N(c3ccc4c(c3)C(C)(C)c3ccccc3-4)c3ccc4c(c3)C(C)(C)c3cc5c(cc3-4)CCCC5)cc21 ; CC1(C)c2cc(N(c3ccccc3)c3ccc4c(c3)C(c3ccccc3)(c3ccccc3)c3ccccc3-4)ccc2-c2cc3c(cc21)CCCC3]

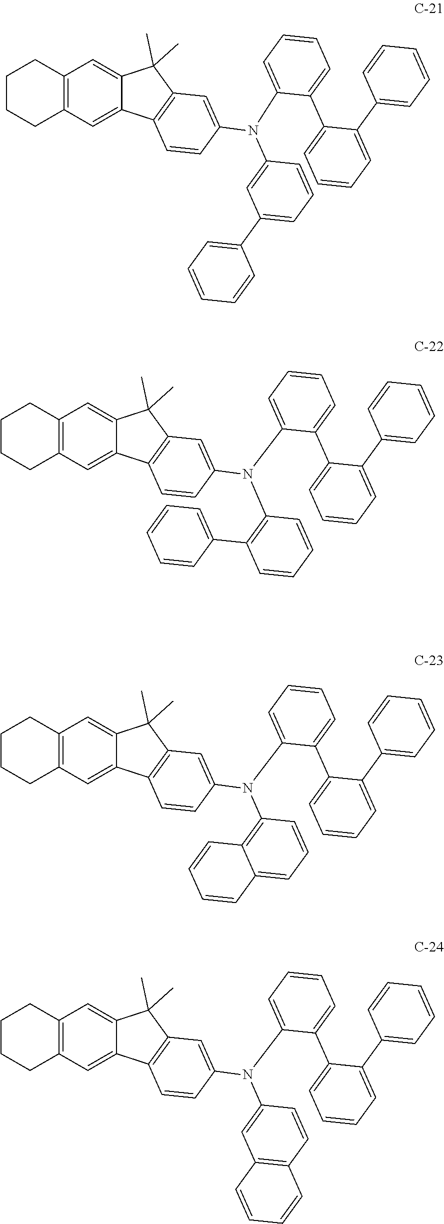 [SMILES: CC1(C)c2cc(N(c3cccc(-c4ccccc4)c3)c3ccccc3-c3ccccc3-c3ccccc3)ccc2-c2cc3c(cc21)CCCC3 ; CC1(C)c2cc(N(c3ccccc3-c3ccccc3)c3ccccc3-c3ccccc3-c3ccccc3)ccc2-c2cc3c(cc21)CCCC3 ; CC1(C)c2cc(N(c3ccccc3-c3ccccc3-c3ccccc3)c3cccc4ccccc34)ccc2-c2cc3c(cc21)CCCC3 ; CC1(C)c2cc(N(c3ccc4ccccc4c3)c3ccccc3-c3ccccc3-c3ccccc3)ccc2-c2cc3c(cc21)CCCC3]

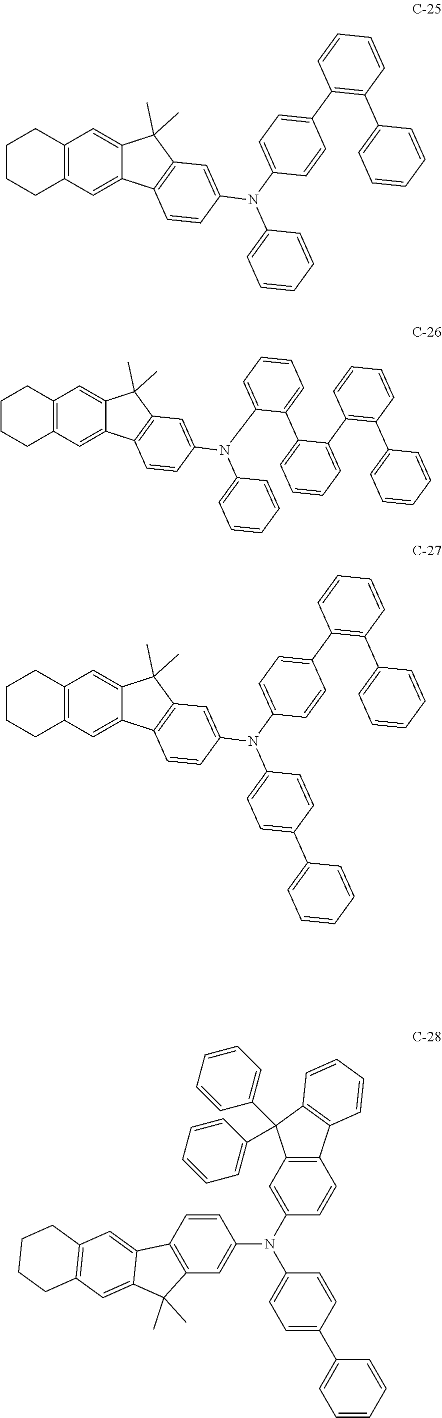 [SMILES: CC1(C)c2cc(N(c3ccccc3)c3ccc(-c4ccccc4-c4ccccc4)cc3)ccc2-c2cc3c(cc21)CCCC3 ; CC1(C)c2cc(N(c3ccccc3)c3ccccc3-c3ccccc3-c3ccccc3-c3ccccc3)ccc2-c2cc3c(cc21)CCCC3 ; CC1(C)c2cc(N(c3ccc(-c4ccccc4)cc3)c3ccc(-c4ccccc4-c4ccccc4)cc3)ccc2-c2cc3c(cc21)CCCC3 ; CC1(C)c2cc(N(c3ccc(-c4ccccc4)cc3)c3ccc4c(c3)C(c3ccccc3)(c3ccccc3)c3ccccc3-4)ccc2-c2cc3c(cc21)CCCC3]

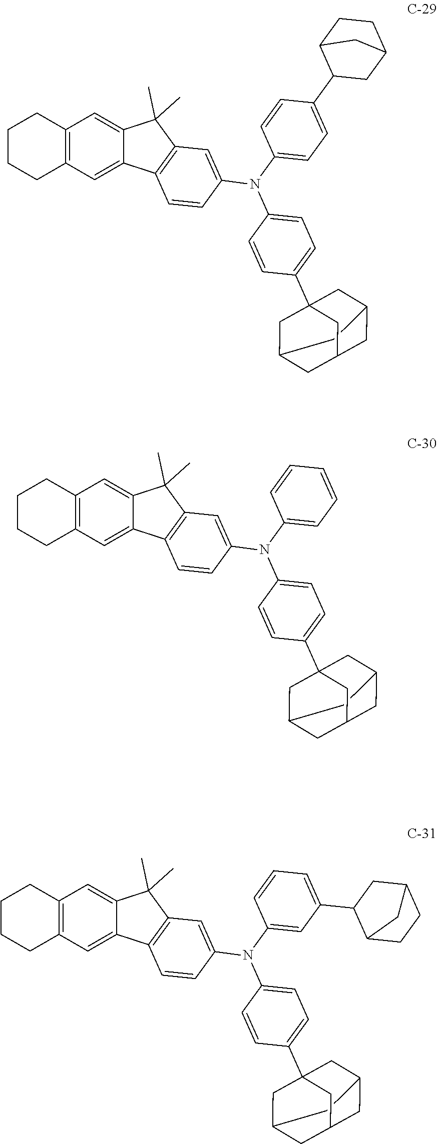 [SMILES: CC1(C)c2cc(N(c3ccc(C4CC5CCC4C5)cc3)c3ccc(C45CC6CC(CC(C6)C4)C5)cc3)ccc2-c2cc3c(cc21)CCCC3 ; CC1(C)c2cc(N(c3ccccc3)c3ccc(C45CC6CC(CC(C6)C4)C5)cc3)ccc2-c2cc3c(cc21)CCCC3 ; CC1(C)c2cc(N(c3ccc(C45CC6CC(CC(C6)C4)C5)cc3)c3cccc(C4CC5CCC4C5)c3)ccc2-c2cc3c(cc21)CCCC3]

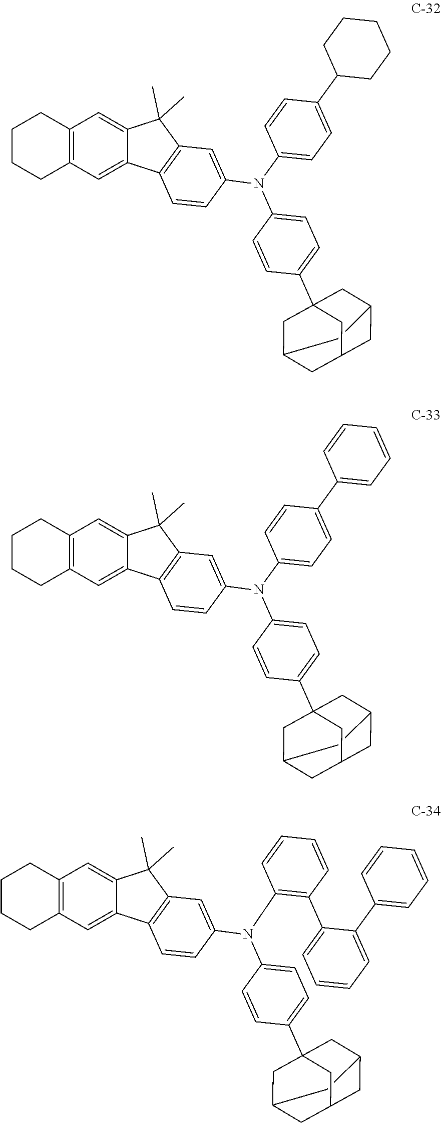 [SMILES: CC1(C)c2cc(N(c3ccc(C4CCCCC4)cc3)c3ccc(C45CC6CC(CC(C6)C4)C5)cc3)ccc2-c2cc3c(cc21)CCCC3 ; CC1(C)c2cc(N(c3ccc(-c4ccccc4)cc3)c3ccc(C45CC6CC(CC(C6)C4)C5)cc3)ccc2-c2cc3c(cc21)CCCC3 ; CC1(C)c2cc(N(c3ccc(C45CC6CC(CC(C6)C4)C5)cc3)c3ccccc3-c3ccccc3-c3ccccc3)ccc2-c2cc3c(cc21)CCCC3]

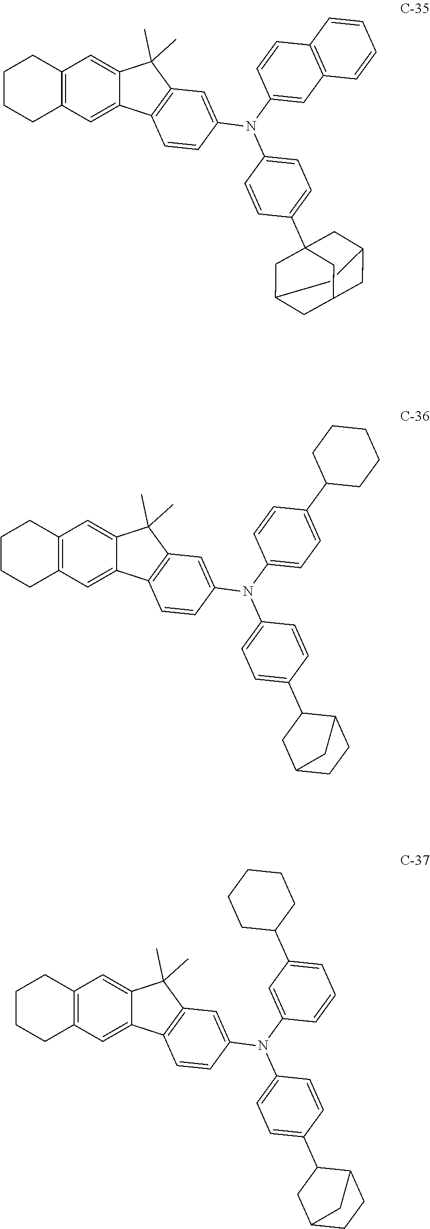 [SMILES: CC1(C)c2cc(N(c3ccc(C45CC6CC(CC(C6)C4)C5)cc3)c3ccc4ccccc4c3)ccc2-c2cc3c(cc21)CCCC3 ; CC1(C)c2cc(N(c3ccc(C4CCCCC4)cc3)c3ccc(C4CC5CCC4C5)cc3)ccc2-c2cc3c(cc21)CCCC3 ; CC1(C)c2cc(N(c3ccc(C4CC5CCC4C5)cc3)c3cccc(C4CCCCC4)c3)ccc2-c2cc3c(cc21)CCCC3]

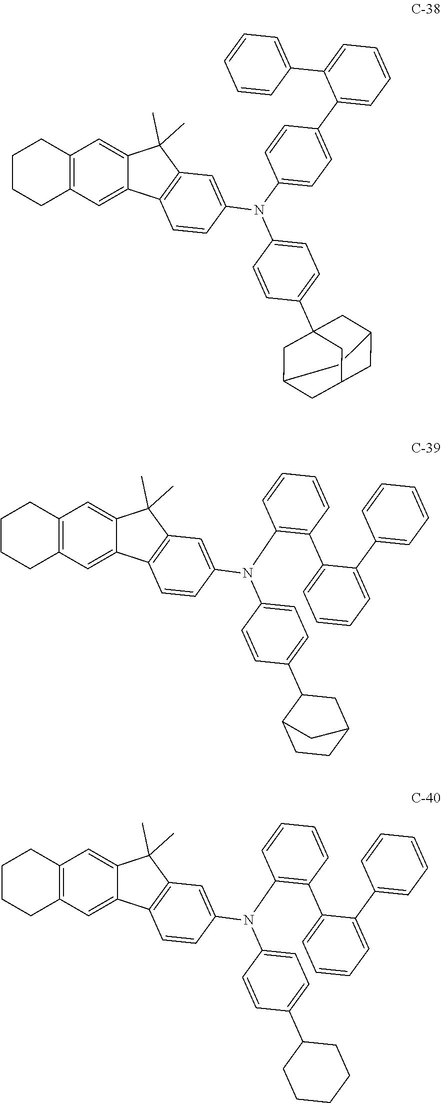 [SMILES: CC1(C)c2cc(N(c3ccc(-c4ccccc4-c4ccccc4)cc3)c3ccc(C45CC6CC(CC(C6)C4)C5)cc3)ccc2-c2cc3c(cc21)CCCC3 ; CC1(C)c2cc(N(c3ccc(C4CC5CCC4C5)cc3)c3ccccc3-c3ccccc3-c3ccccc3)ccc2-c2cc3c(cc21)CCCC3 ; CC1(C)c2cc(N(c3ccc(C4CCCCC4)cc3)c3ccccc3-c3ccccc3-c3ccccc3)ccc2-c2cc3c(cc21)CCCC3]

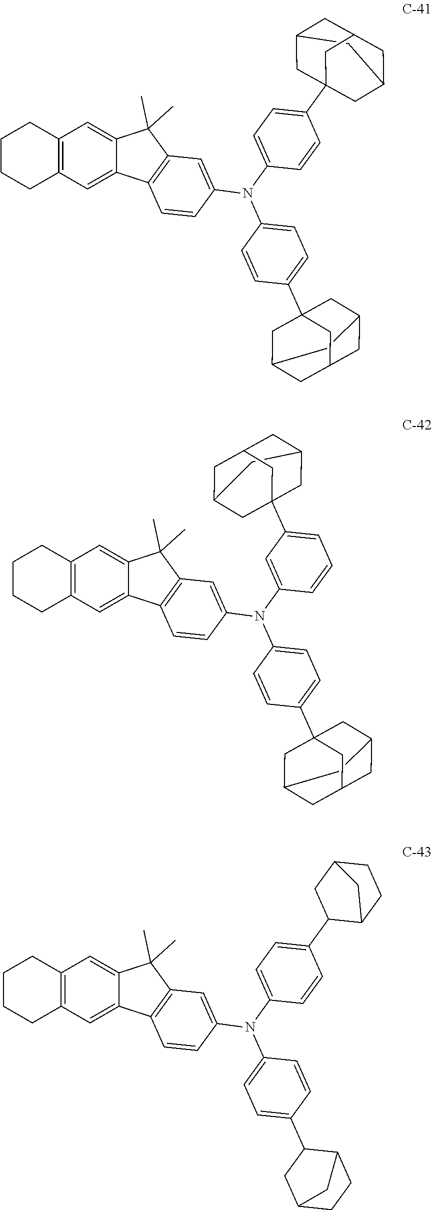 [SMILES: CC1(C)c2cc(N(c3ccc(C45CC6CC(CC(C6)C4)C5)cc3)c3ccc(C45CC6CC(CC(C6)C4)C5)cc3)ccc2-c2cc3c(cc21)CCCC3 ; CC1(C)c2cc(N(c3ccc(C45CC6CC(CC(C6)C4)C5)cc3)c3cccc(C45CC6CC(CC(C6)C4)C5)c3)ccc2-c2cc3c(cc21)CCCC3 ; CC1(C)c2cc(N(c3ccc(C4CC5CCC4C5)cc3)c3ccc(C4CC5CCC4C5)cc3)ccc2-c2cc3c(cc21)CCCC3]

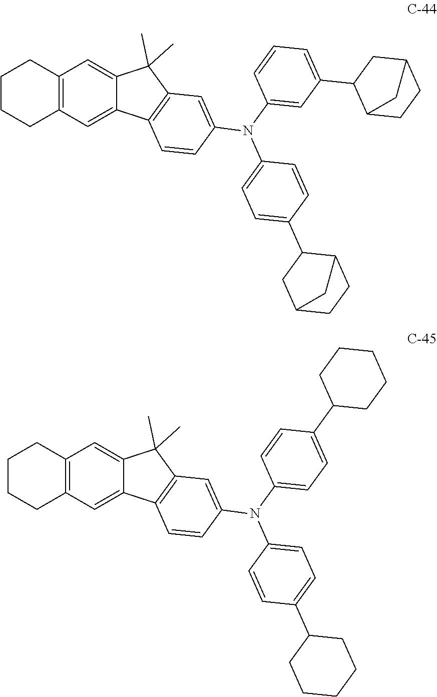

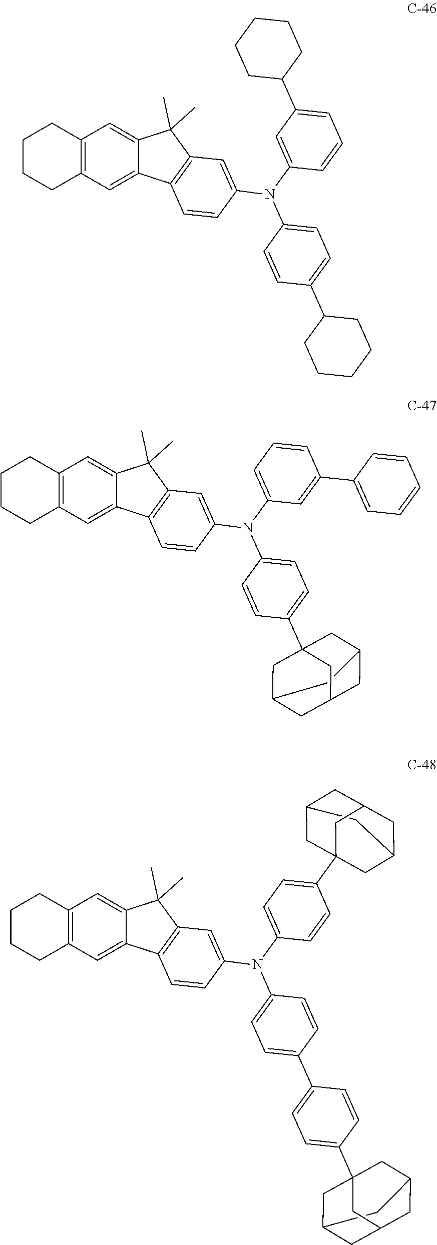 [SMILES: CC1(C)c2cc(N(c3ccc(C4CCCCC4)cc3)c3cccc(C4CCCCC4)c3)ccc2-c2cc3c(cc21)CCCC3 ; CC1(C)c2cc(N(c3ccc(C45CC6CC(CC(C6)C4)C5)cc3)c3cccc(-c4ccccc4)c3)ccc2-c2cc3c(cc21)CCCC3 ; CC1(C)c2cc(N(c3ccc(-c4ccc(C56CC7CC(CC(C7)C5)C6)cc4)cc3)c3ccc(C45CC6CC(CC(C6)C4)C5)cc3)ccc2-c2cc3c(cc21)CCCC3]

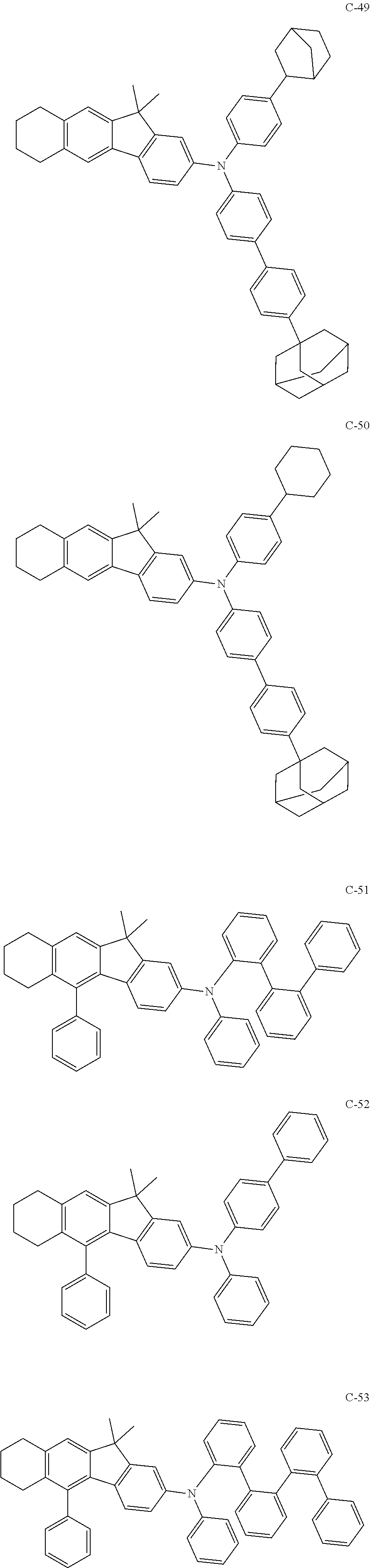 [SMILES: CC1(C)c2cc(N(c3ccc(-c4ccc(C56CC7CC(CC(C7)C5)C6)cc4)cc3)c3ccc(C4CC5CCC4C5)cc3)ccc2-c2cc3c(cc21)CCCC3 ; CC1(C)c2cc(N(c3ccc(-c4ccc(C56CC7CC(CC(C7)C5)C6)cc4)cc3)c3ccc(C4CCCCC4)cc3)ccc2-c2cc3c(cc21)CCCC3 ; CC1(C)c2cc(N(c3ccccc3)c3ccccc3-c3ccccc3-c3ccccc3)ccc2-c2c1cc1c(c2-c2ccccc2)CCCC1 ; CC1(C)c2cc(N(c3ccccc3)c3ccc(-c4ccccc4)cc3)ccc2-c2c1cc1c(c2-c2ccccc2)CCCC1 ; CC1(C)c2cc(N(c3ccccc3)c3ccccc3-c3ccccc3-c3ccccc3-c3ccccc3)ccc2-c2c1cc1c(c2-c2ccccc2)CCCC1]

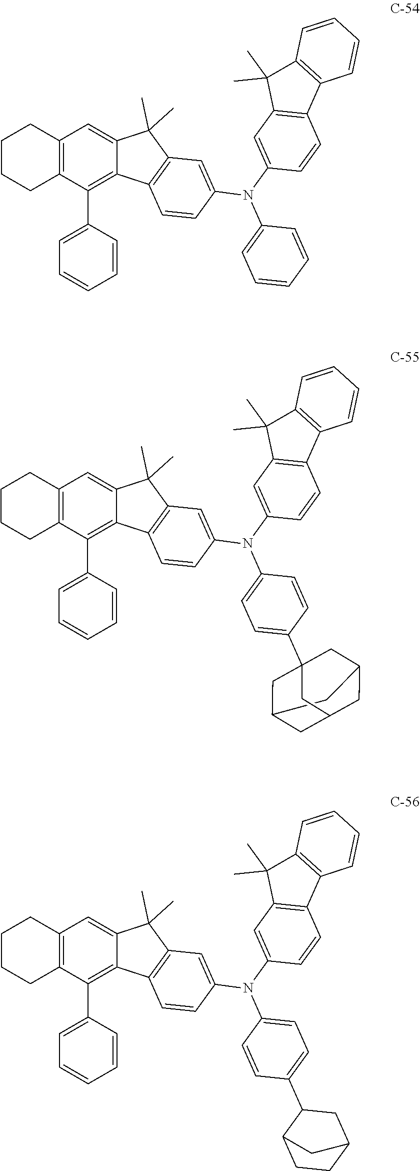 [SMILES: CC1(C)c2ccccc2-c2ccc(N(c3ccccc3)c3ccc4c(c3)C(C)(C)c3cc5c(c(-c6ccccc6)c3-4)CCCC5)cc21 ; CC1(C)c2ccccc2-c2ccc(N(c3ccc(C45CC6CC(CC(C6)C4)C5)cc3)c3ccc4c(c3)C(C)(C)c3cc5c(c(-c6ccccc6)c3-4)CCCC5)cc21 ; CC1(C)c2ccccc2-c2ccc(N(c3ccc(C4CC5CCC4C5)cc3)c3ccc4c(c3)C(C)(C)c3cc5c(c(-c6ccccc6)c3-4)CCCC5)cc21]

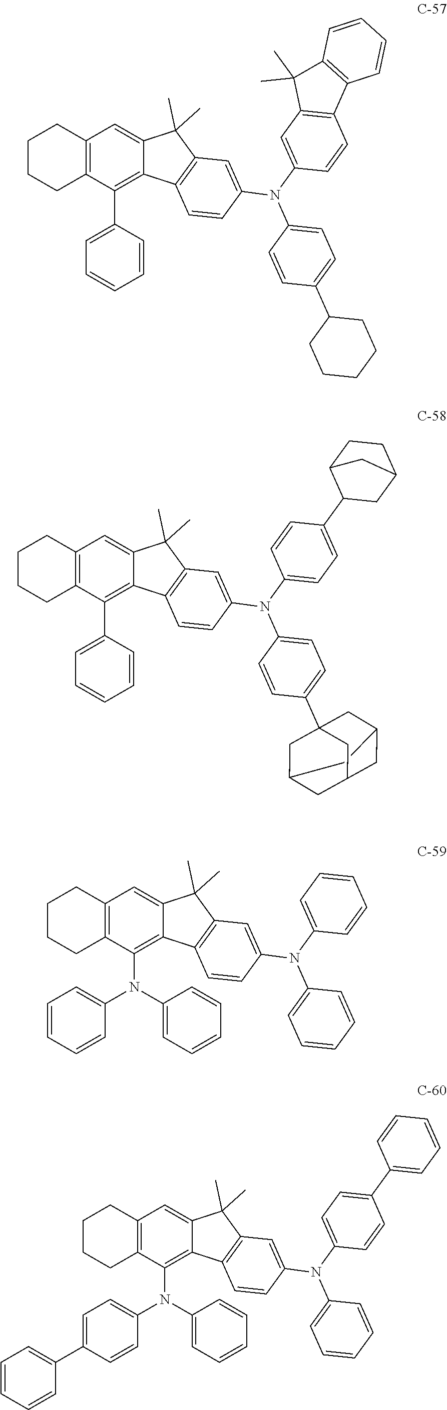 [SMILES: CC1(C)c2ccccc2-c2ccc(N(c3ccc(C4CCCCC4)cc3)c3ccc4c(c3)C(C)(C)c3cc5c(c(-c6ccccc6)c3-4)CCCC5)cc21 ; CC1(C)c2cc(N(c3ccc(C4CC5CCC4C5)cc3)c3ccc(C45CC6CC(CC(C6)C4)C5)cc3)ccc2-c2c1cc1c(c2-c2ccccc2)CCCC1 ; CC1(C)c2cc(N(c3ccccc3)c3ccccc3)ccc2-c2c1cc1c(c2N(c2ccccc2)c2ccccc2)CCCC1 ; CC1(C)c2cc(N(c3ccccc3)c3ccc(-c4ccccc4)cc3)ccc2-c2c1cc1c(c2N(c2ccccc2)c2ccc(-c3ccccc3)cc2)CCCC1]

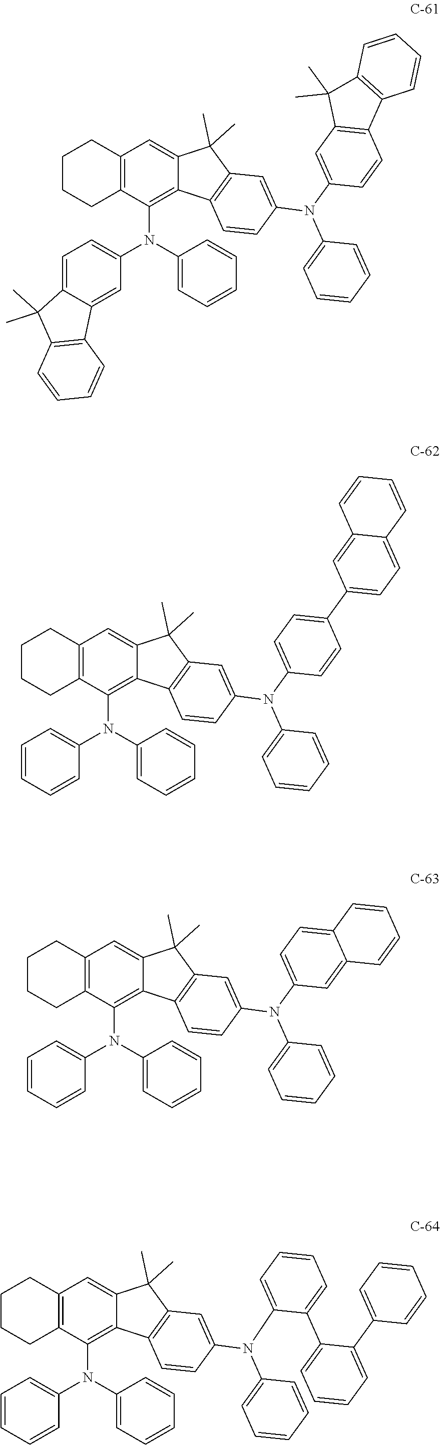 [SMILES: CC1(C)c2ccccc2-c2cc(N(c3ccccc3)c3c4c(cc5c3-c3ccc(N(c6ccccc6)c6ccc7c(c6)C(C)(C)c6ccccc6-7)cc3C5(C)C)CCCC4)ccc21 ; CC1(C)c2cc(N(c3ccccc3)c3ccc(-c4ccc5ccccc5c4)cc3)ccc2-c2c1cc1c(c2N(c2ccccc2)c2ccccc2)CCCC1 ; CC1(C)c2cc(N(c3ccccc3)c3ccc4ccccc4c3)ccc2-c2c1cc1c(c2N(c2ccccc2)c2ccccc2)CCCC1 ; CC1(C)c2cc(N(c3ccccc3)c3ccccc3-c3ccccc3-c3ccccc3)ccc2-c2c1cc1c(c2N(c2ccccc2)c2ccccc2)CCCC1]

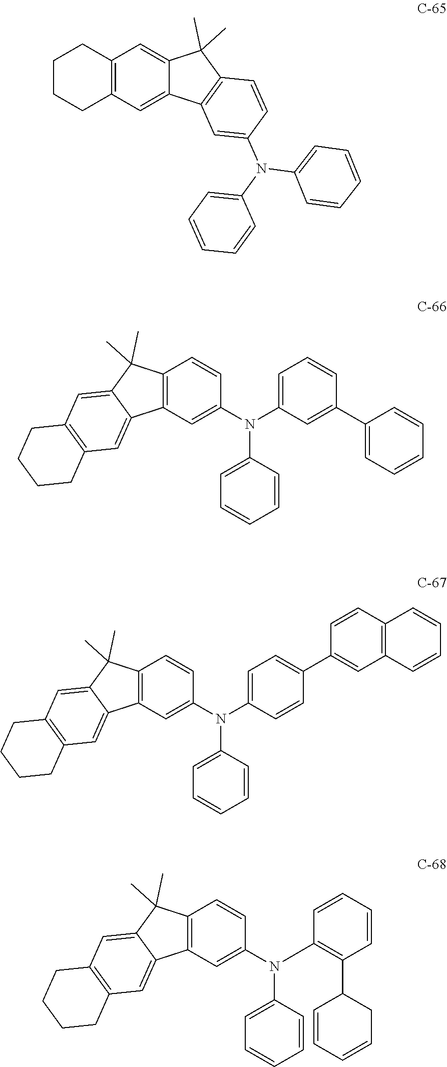 [SMILES: CC1(C)c2ccc(N(c3ccccc3)c3ccccc3)cc2-c2cc3c(cc21)CCCC3 ; CC1(C)c2ccc(N(c3ccccc3)c3cccc(-c4ccccc4)c3)cc2-c2cc3c(cc21)CCCC3 ; CC1(C)c2ccc(N(c3ccccc3)c3ccc(-c4ccc5ccccc5c4)cc3)cc2-c2cc3c(cc21)CCCC3 ; CC1(C)c2ccc(N(c3ccccc3)c3ccccc3C3C=CC=CC3)cc2-c2cc3c(cc21)CCCC3]

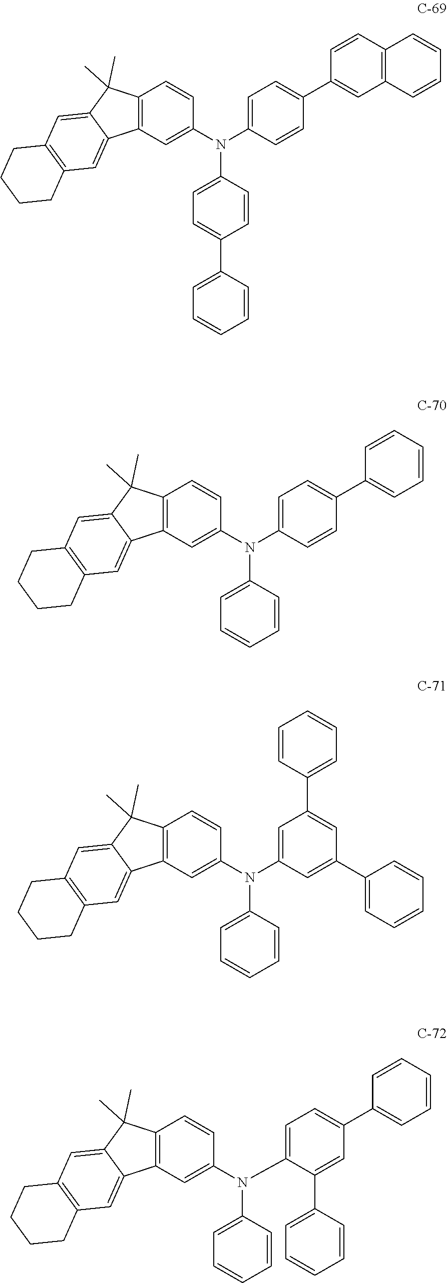 [SMILES: CC1(C)c2ccc(N(c3ccc(-c4ccccc4)cc3)c3ccc(-c4ccc5ccccc5c4)cc3)cc2-c2cc3c(cc21)CCCC3 ; CC1(C)c2ccc(N(c3ccccc3)c3ccc(-c4ccccc4)cc3)cc2-c2cc3c(cc21)CCCC3 ; CC1(C)c2ccc(N(c3ccccc3)c3cc(-c4ccccc4)cc(-c4ccccc4)c3)cc2-c2cc3c(cc21)CCCC3 ; CC1(C)c2ccc(N(c3ccccc3)c3ccc(-c4ccccc4)cc3-c3ccccc3)cc2-c2cc3c(cc21)CCCC3]

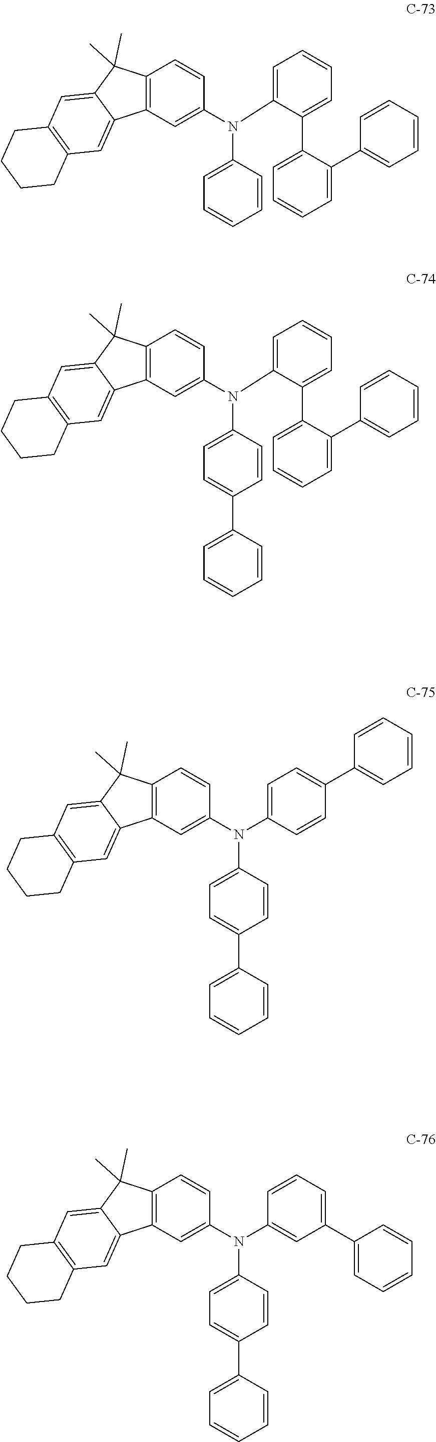 [SMILES: CC1(C)c2ccc(N(c3ccccc3)c3ccccc3-c3ccccc3-c3ccccc3)cc2-c2cc3c(cc21)CCCC3 ; CC1(C)c2ccc(N(c3ccc(-c4ccccc4)cc3)c3ccccc3-c3ccccc3-c3ccccc3)cc2-c2cc3c(cc21)CCCC3 ; CC1(C)c2ccc(N(c3ccc(-c4ccccc4)cc3)c3ccc(-c4ccccc4)cc3)cc2-c2cc3c(cc21)CCCC3 ; CC1(C)c2ccc(N(c3ccc(-c4ccccc4)cc3)c3cccc(-c4ccccc4)c3)cc2-c2cc3c(cc21)CCCC3]

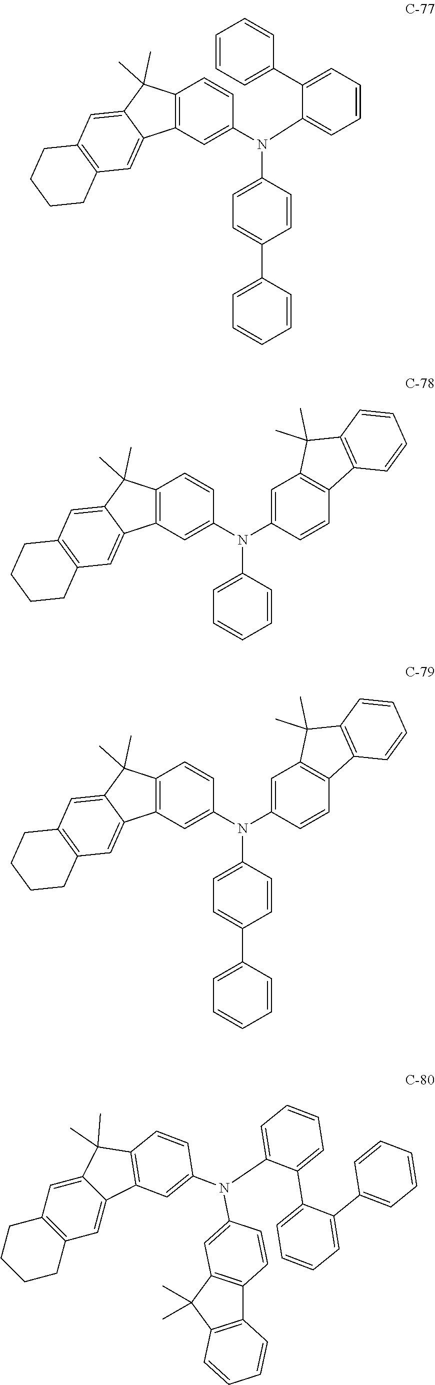 [SMILES: CC1(C)c2ccc(N(c3ccc(-c4ccccc4)cc3)c3ccccc3-c3ccccc3)cc2-c2cc3c(cc21)CCCC3 ; CC1(C)c2ccccc2-c2ccc(N(c3ccccc3)c3ccc4c(c3)-c3cc5c(cc3C4(C)C)CCCC5)cc21 ; CC1(C)c2ccccc2-c2ccc(N(c3ccc(-c4ccccc4)cc3)c3ccc4c(c3)-c3cc5c(cc3C4(C)C)CCCC5)cc21 ; CC1(C)c2ccccc2-c2ccc(N(c3ccc4c(c3)-c3cc5c(cc3C4(C)C)CCCC5)c3ccccc3-c3ccccc3-c3ccccc3)cc21]

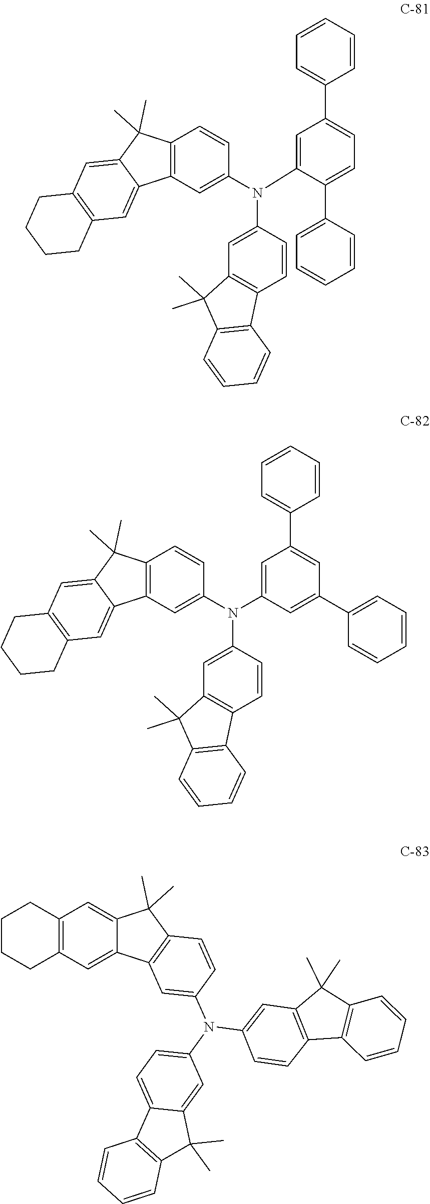 [SMILES: CC1(C)c2ccccc2-c2ccc(N(c3ccc4c(c3)-c3cc5c(cc3C4(C)C)CCCC5)c3cc(-c4ccccc4)ccc3-c3ccccc3)cc21 ; CC1(C)c2ccccc2-c2ccc(N(c3cc(-c4ccccc4)cc(-c4ccccc4)c3)c3ccc4c(c3)-c3cc5c(cc3C4(C)C)CCCC5)cc21 ; CC1(C)c2ccccc2-c2ccc(N(c3ccc4c(c3)-c3cc5c(cc3C4(C)C)CCCC5)c3ccc4c(c3)C(C)(C)c3ccccc3-4)cc21]

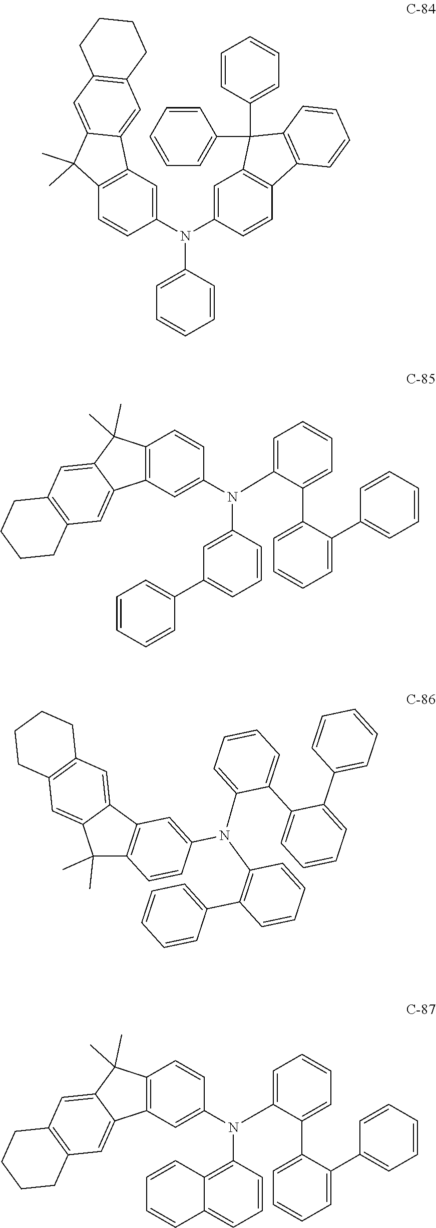 [SMILES: CC1(C)c2ccc(N(c3ccccc3)c3ccc4c(c3)C(c3ccccc3)(c3ccccc3)c3ccccc3-4)cc2-c2cc3c(cc21)CCCC3 ; CC1(C)c2ccc(N(c3cccc(-c4ccccc4)c3)c3ccccc3-c3ccccc3-c3ccccc3)cc2-c2cc3c(cc21)CCCC3 ; CC1(C)c2ccc(N(c3ccccc3-c3ccccc3)c3ccccc3-c3ccccc3-c3ccccc3)cc2-c2cc3c(cc21)CCCC3 ; CC1(C)c2ccc(N(c3ccccc3-c3ccccc3-c3ccccc3)c3cccc4ccccc34)cc2-c2cc3c(cc21)CCCC3]

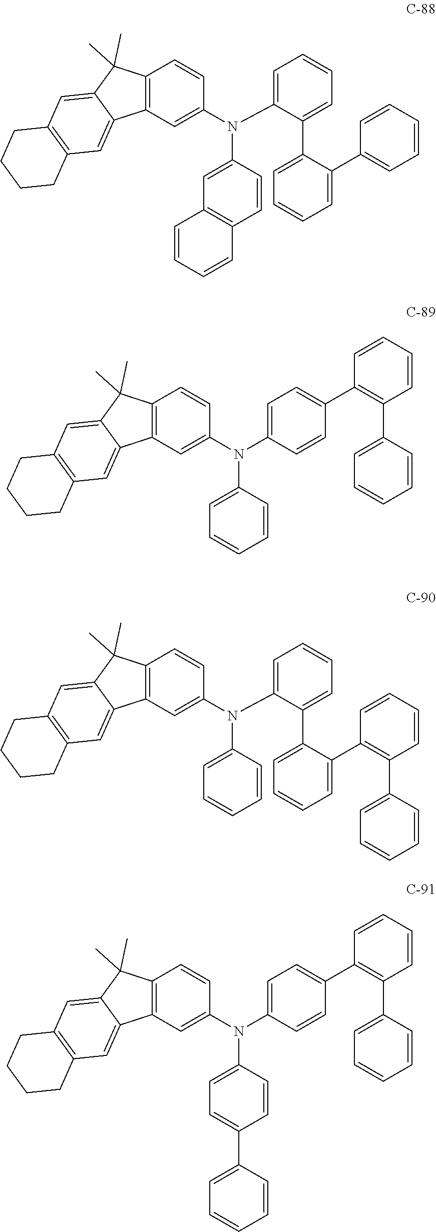 [SMILES: CC1(C)c2ccc(N(c3ccc4ccccc4c3)c3ccccc3-c3ccccc3-c3ccccc3)cc2-c2cc3c(cc21)CCCC3 ; CC1(C)c2ccc(N(c3ccccc3)c3ccc(-c4ccccc4-c4ccccc4)cc3)cc2-c2cc3c(cc21)CCCC3 ; CC1(C)c2ccc(N(c3ccccc3)c3ccccc3-c3ccccc3-c3ccccc3-c3ccccc3)cc2-c2cc3c(cc21)CCCC3 ; CC1(C)c2ccc(N(c3ccc(-c4ccccc4)cc3)c3ccc(-c4ccccc4-c4ccccc4)cc3)cc2-c2cc3c(cc21)CCCC3]

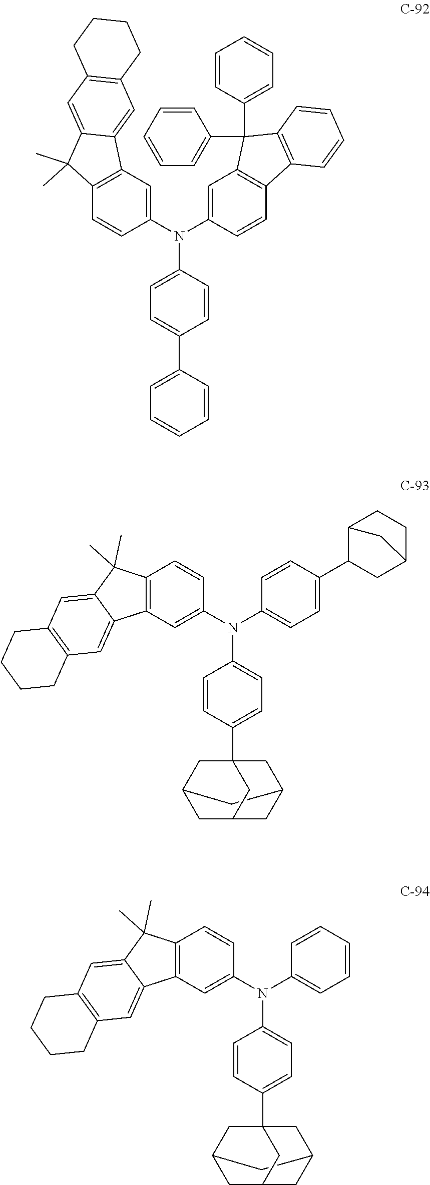 [SMILES: CC1(C)c2ccc(N(c3ccc(-c4ccccc4)cc3)c3ccc4c(c3)C(c3ccccc3)(c3ccccc3)c3ccccc3-4)cc2-c2cc3c(cc21)CCCC3 ; CC1(C)c2ccc(N(c3ccc(C4CC5CCC4C5)cc3)c3ccc(C45CC6CC(CC(C6)C4)C5)cc3)cc2-c2cc3c(cc21)CCCC3 ; CC1(C)c2ccc(N(c3ccccc3)c3ccc(C45CC6CC(CC(C6)C4)C5)cc3)cc2-c2cc3c(cc21)CCCC3]

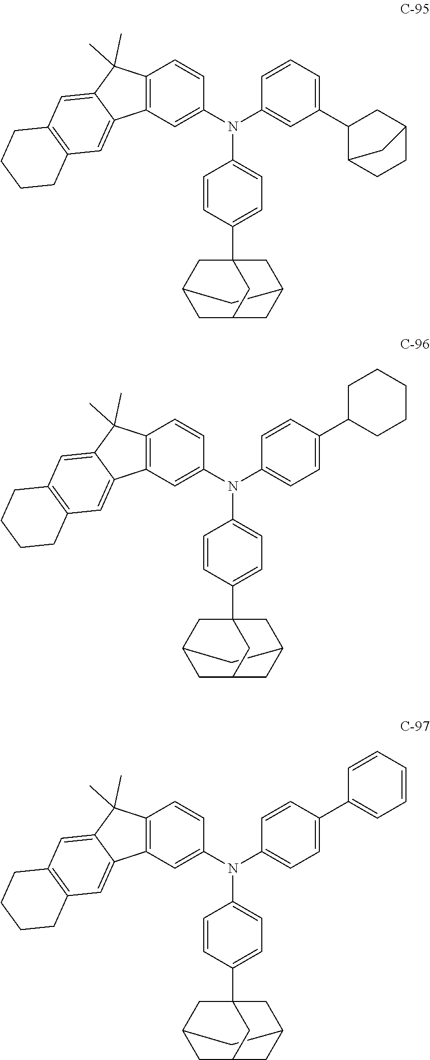 [SMILES: CC1(C)c2ccc(N(c3ccc(C45CC6CC(CC(C6)C4)C5)cc3)c3cccc(C4CC5CCC4C5)c3)cc2-c2cc3c(cc21)CCCC3 ; CC1(C)c2ccc(N(c3ccc(C4CCCCC4)cc3)c3ccc(C45CC6CC(CC(C6)C4)C5)cc3)cc2-c2cc3c(cc21)CCCC3 ; CC1(C)c2ccc(N(c3ccc(-c4ccccc4)cc3)c3ccc(C45CC6CC(CC(C6)C4)C5)cc3)cc2-c2cc3c(cc21)CCCC3]

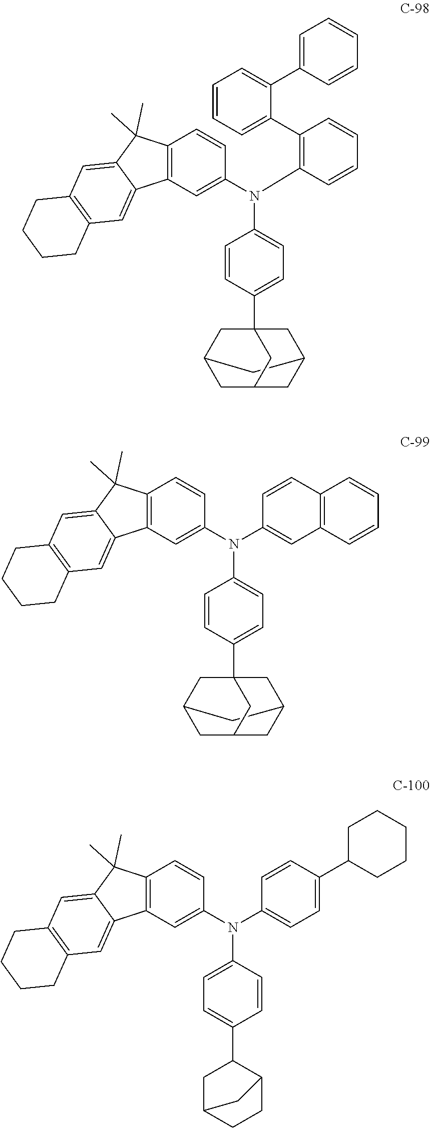 [SMILES: CC1(C)c2ccc(N(c3ccc(C45CC6CC(CC(C6)C4)C5)cc3)c3ccccc3-c3ccccc3-c3ccccc3)cc2-c2cc3c(cc21)CCCC3 ; CC1(C)c2ccc(N(c3ccc(C45CC6CC(CC(C6)C4)C5)cc3)c3ccc4ccccc4c3)cc2-c2cc3c(cc21)CCCC3 ; CC1(C)c2ccc(N(c3ccc(C4CCCCC4)cc3)c3ccc(C4CC5CCC4C5)cc3)cc2-c2cc3c(cc21)CCCC3]

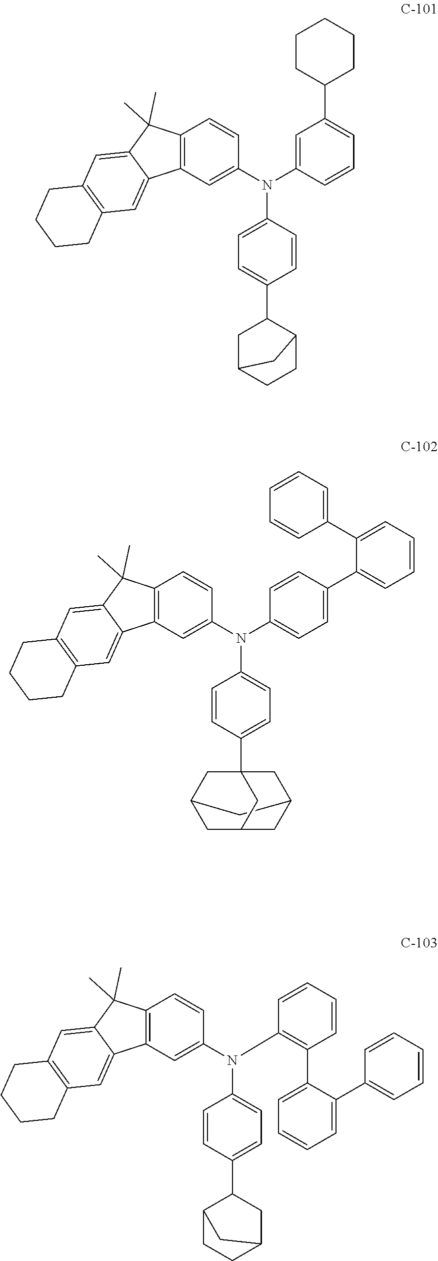 [SMILES: CC1(C)c2ccc(N(c3ccc(C4CC5CCC4C5)cc3)c3cccc(C4CCCCC4)c3)cc2-c2cc3c(cc21)CCCC3 ; CC1(C)c2ccc(N(c3ccc(-c4ccccc4-c4ccccc4)cc3)c3ccc(C45CC6CC(CC(C6)C4)C5)cc3)cc2-c2cc3c(cc21)CCCC3 ; CC1(C)c2ccc(N(c3ccc(C4CC5CCC4C5)cc3)c3ccccc3-c3ccccc3-c3ccccc3)cc2-c2cc3c(cc21)CCCC3]

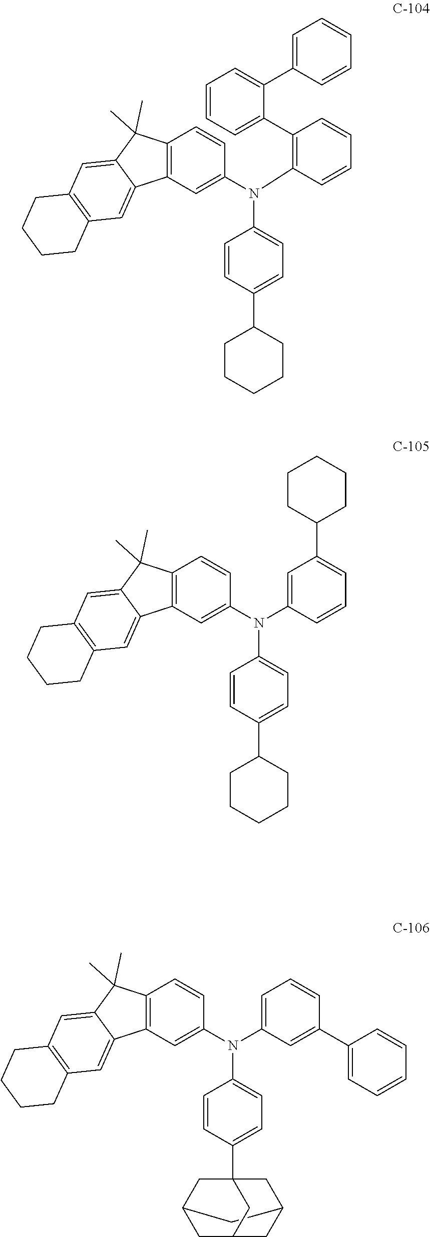 [SMILES: CC1(C)c2ccc(N(c3ccc(C4CCCCC4)cc3)c3ccccc3-c3ccccc3-c3ccccc3)cc2-c2cc3c(cc21)CCCC3 ; CC1(C)c2ccc(N(c3ccc(C4CCCCC4)cc3)c3cccc(C4CCCCC4)c3)cc2-c2cc3c(cc21)CCCC3 ; CC1(C)c2ccc(N(c3ccc(C45CC6CC(CC(C6)C4)C5)cc3)c3cccc(-c4ccccc4)c3)cc2-c2cc3c(cc21)CCCC3]

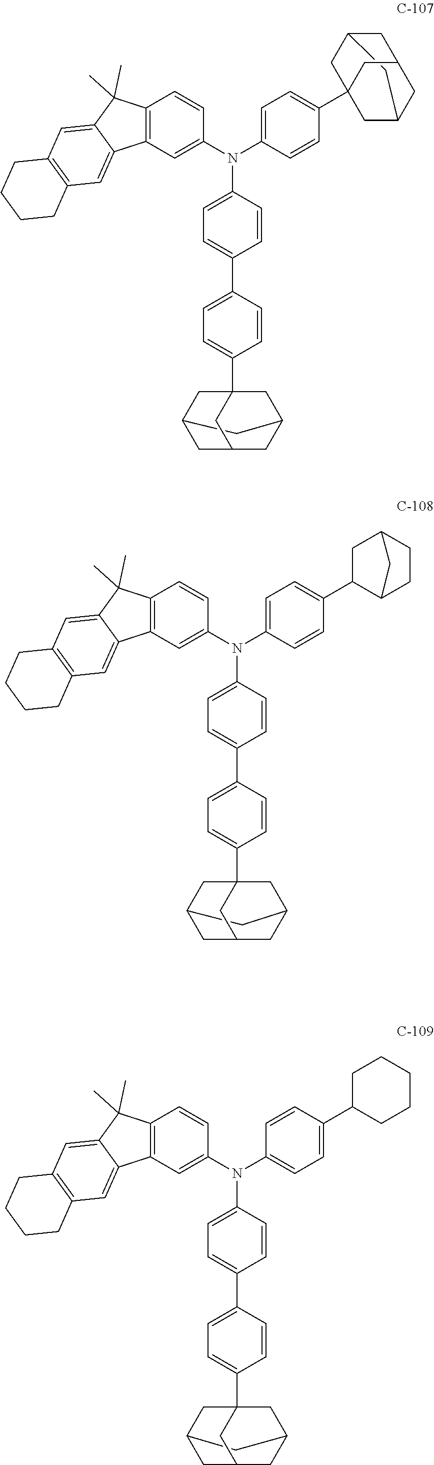 [SMILES: CC1(C)c2ccc(N(c3ccc(-c4ccc(C56CC7CC(CC(C7)C5)C6)cc4)cc3)c3ccc(C45CC6CC(CC(C6)C4)C5)cc3)cc2-c2cc3c(cc21)CCCC3 ; CC1(C)c2ccc(N(c3ccc(-c4ccc(C56CC7CC(CC(C7)C5)C6)cc4)cc3)c3ccc(C4CC5CCC4C5)cc3)cc2-c2cc3c(cc21)CCCC3 ; CC1(C)c2ccc(N(c3ccc(-c4ccc(C56CC7CC(CC(C7)C5)C6)cc4)cc3)c3ccc(C4CCCCC4)cc3)cc2-c2cc3c(cc21)CCCC3]

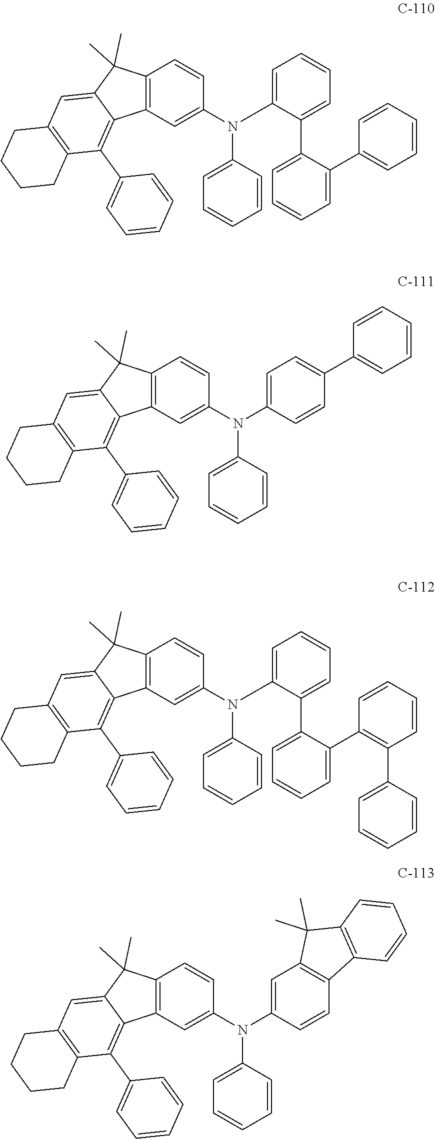 [SMILES: CC1(C)c2ccc(N(c3ccccc3)c3ccccc3-c3ccccc3-c3ccccc3)cc2-c2c1cc1c(c2-c2ccccc2)CCCC1 ; CC1(C)c2ccc(N(c3ccccc3)c3ccc(-c4ccccc4)cc3)cc2-c2c1cc1c(c2-c2ccccc2)CCCC1 ; CC1(C)c2ccc(N(c3ccccc3)c3ccccc3-c3ccccc3-c3ccccc3-c3ccccc3)cc2-c2c1cc1c(c2-c2ccccc2)CCCC1 ; CC1(C)c2ccccc2-c2ccc(N(c3ccccc3)c3ccc4c(c3)-c3c(cc5c(c3-c3ccccc3)CCCC5)C4(C)C)cc21]

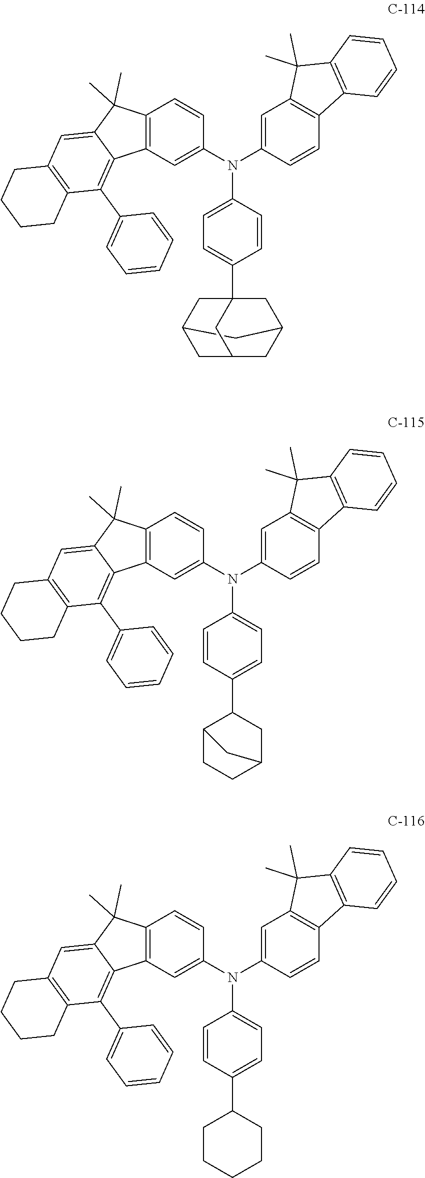 [SMILES: CC1(C)c2ccccc2-c2ccc(N(c3ccc(C45CC6CC(CC(C6)C4)C5)cc3)c3ccc4c(c3)-c3c(cc5c(c3-c3ccccc3)CCCC5)C4(C)C)cc21 ; CC1(C)c2ccccc2-c2ccc(N(c3ccc(C4CC5CCC4C5)cc3)c3ccc4c(c3)-c3c(cc5c(c3-c3ccccc3)CCCC5)C4(C)C)cc21 ; CC1(C)c2ccccc2-c2ccc(N(c3ccc(C4CCCCC4)cc3)c3ccc4c(c3)-c3c(cc5c(c3-c3ccccc3)CCCC5)C4(C)C)cc21]

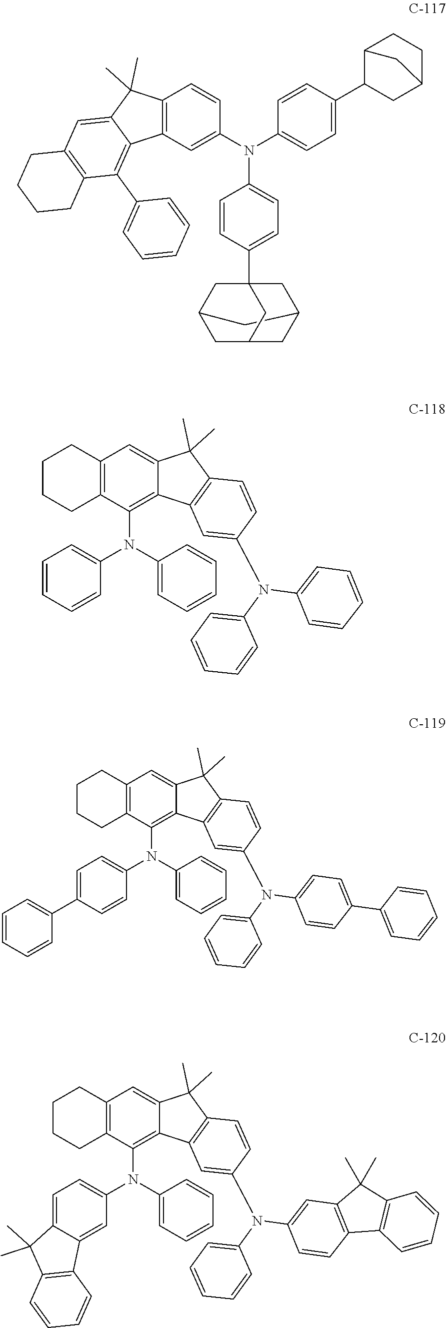 [SMILES: CC1(C)c2ccc(N(c3ccc(C4CC5CCC4C5)cc3)c3ccc(C45CC6CC(CC(C6)C4)C5)cc3)cc2-c2c1cc1c(c2-c2ccccc2)CCCC1 ; CC1(C)c2ccc(N(c3ccccc3)c3ccccc3)cc2-c2c1cc1c(c2N(c2ccccc2)c2ccccc2)CCCC1 ; CC1(C)c2ccc(N(c3ccccc3)c3ccc(-c4ccccc4)cc3)cc2-c2c1cc1c(c2N(c2ccccc2)c2ccc(-c3ccccc3)cc2)CCCC1 ; CC1(C)c2ccccc2-c2cc(N(c3ccccc3)c3c4c(cc5c3-c3cc(N(c6ccccc6)c6ccc7c(c6)C(C)(C)c6ccccc6-7)ccc3C5(C)C)CCCC4)ccc21]

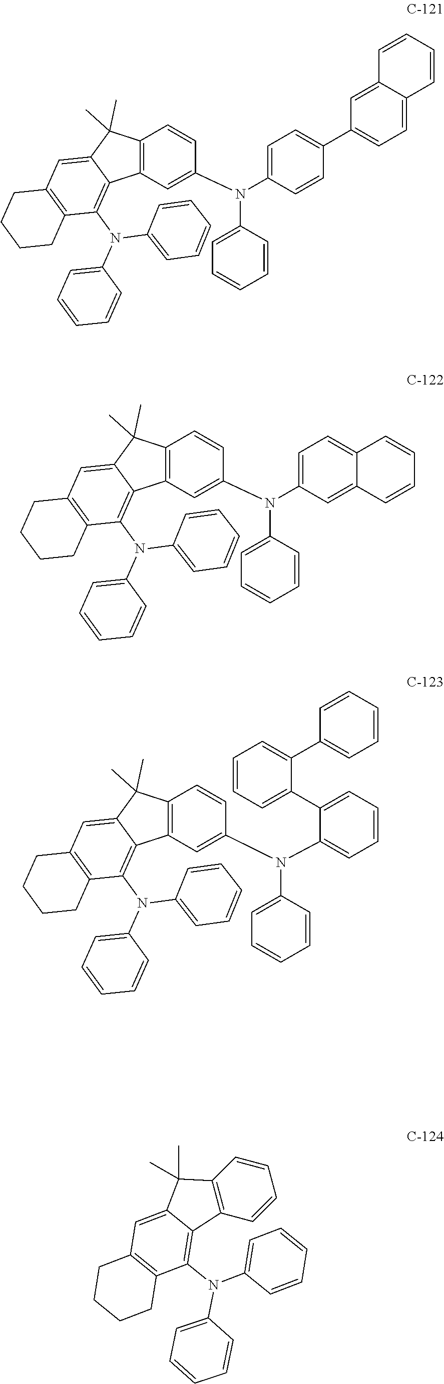 [SMILES: CC1(C)c2ccc(N(c3ccccc3)c3ccc(-c4ccc5ccccc5c4)cc3)cc2-c2c1cc1c(c2N(c2ccccc2)c2ccccc2)CCCC1 ; CC1(C)c2ccc(N(c3ccccc3)c3ccc4ccccc4c3)cc2-c2c1cc1c(c2N(c2ccccc2)c2ccccc2)CCCC1 ; CC1(C)c2ccc(N(c3ccccc3)c3ccccc3-c3ccccc3-c3ccccc3)cc2-c2c1cc1c(c2N(c2ccccc2)c2ccccc2)CCCC1 ; CC1(C)c2ccccc2-c2c1cc1c(c2N(c2ccccc2)c2ccccc2)CCCC1]

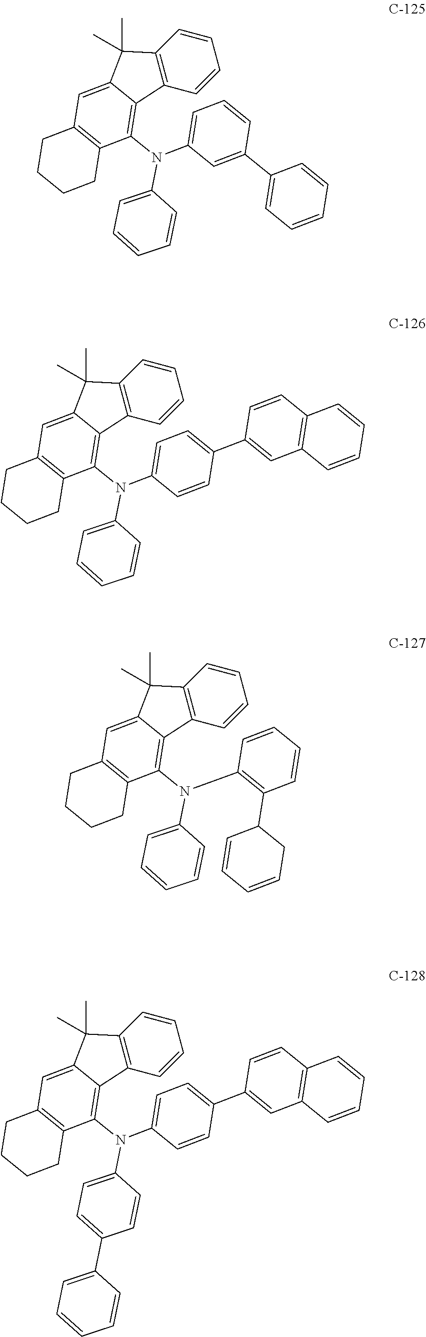 [SMILES: CC1(C)c2ccccc2-c2c1cc1c(c2N(c2ccccc2)c2cccc(-c3ccccc3)c2)CCCC1 ; CC1(C)c2ccccc2-c2c1cc1c(c2N(c2ccccc2)c2ccc(-c3ccc4ccccc4c3)cc2)CCCC1 ; CC1(C)c2ccccc2-c2c1cc1c(c2N(c2ccccc2)c2ccccc2C2C=CC=CC2)CCCC1 ; CC1(C)c2ccccc2-c2c1cc1c(c2N(c2ccc(-c3ccccc3)cc2)c2ccc(-c3ccc4ccccc4c3)cc2)CCCC1]

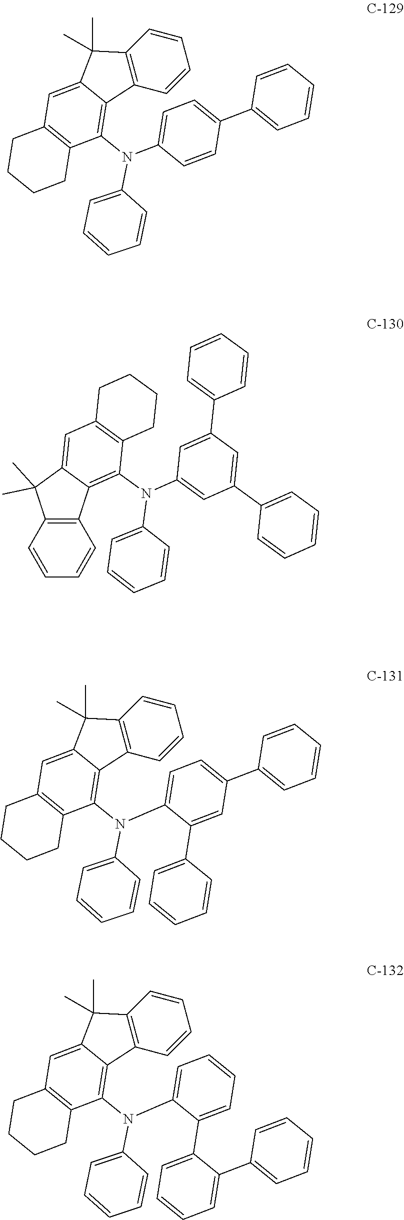 [SMILES: CC1(C)c2ccccc2-c2c1cc1c(c2N(c2ccccc2)c2ccc(-c3ccccc3)cc2)CCCC1 ; CC1(C)c2ccccc2-c2c1cc1c(c2N(c2ccccc2)c2cc(-c3ccccc3)cc(-c3ccccc3)c2)CCCC1 ; CC1(C)c2ccccc2-c2c1cc1c(c2N(c2ccccc2)c2ccc(-c3ccccc3)cc2-c2ccccc2)CCCC1 ; CC1(C)c2ccccc2-c2c1cc1c(c2N(c2ccccc2)c2ccccc2-c2ccccc2-c2ccccc2)CCCC1]

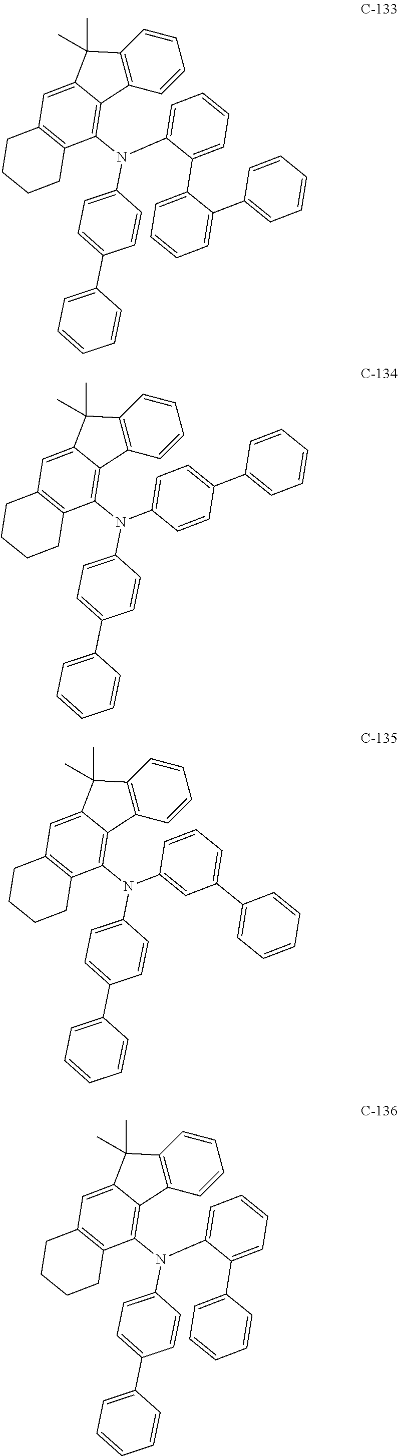

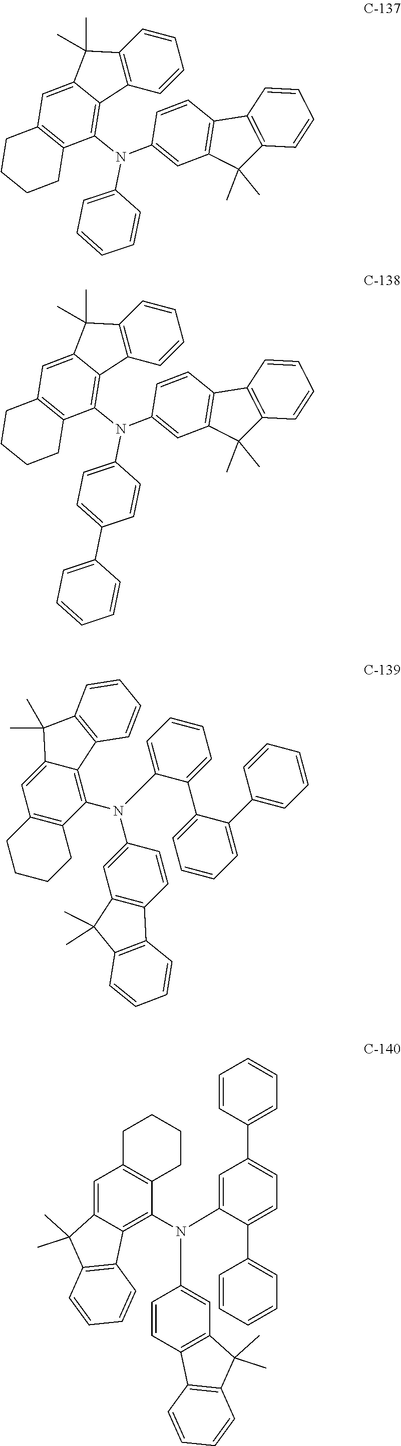

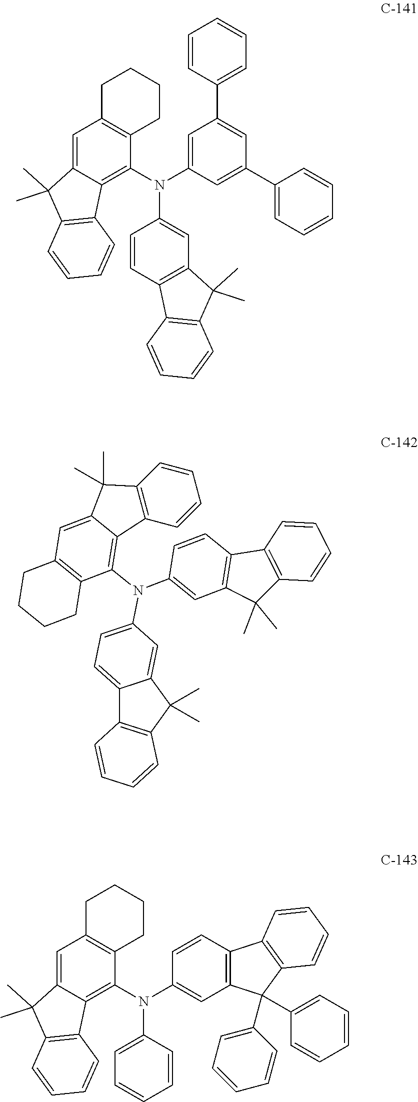 [SMILES: CC1(C)c2ccccc2-c2ccc(N(c3cc(-c4ccccc4)cc(-c4ccccc4)c3)c3c4c(cc5c3-c3ccccc3C5(C)C)CCCC4)cc21 ; CC1(C)c2ccccc2-c2ccc(N(c3ccc4c(c3)C(C)(C)c3ccccc3-4)c3c4c(cc5c3-c3ccccc3C5(C)C)CCCC4)cc21 ; CC1(C)c2ccccc2-c2c1cc1c(c2N(c2ccccc2)c2ccc3c(c2)C(c2ccccc2)(c2ccccc2)c2ccccc2-3)CCCC1]

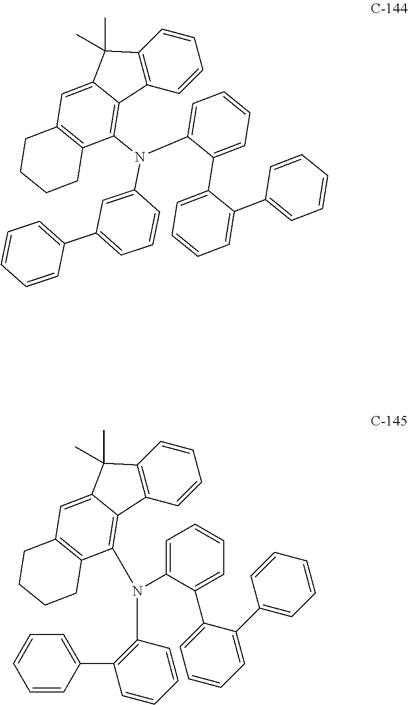 [SMILES: CC1(C)c2ccccc2-c2c1cc1c(c2N(c2cccc(-c3ccccc3)c2)c2ccccc2-c2ccccc2-c2ccccc2)CCCC1 ; CC1(C)c2ccccc2-c2c1cc1c(c2N(c2ccccc2-c2ccccc2)c2ccccc2-c2ccccc2-c2ccccc2)CCCC1]

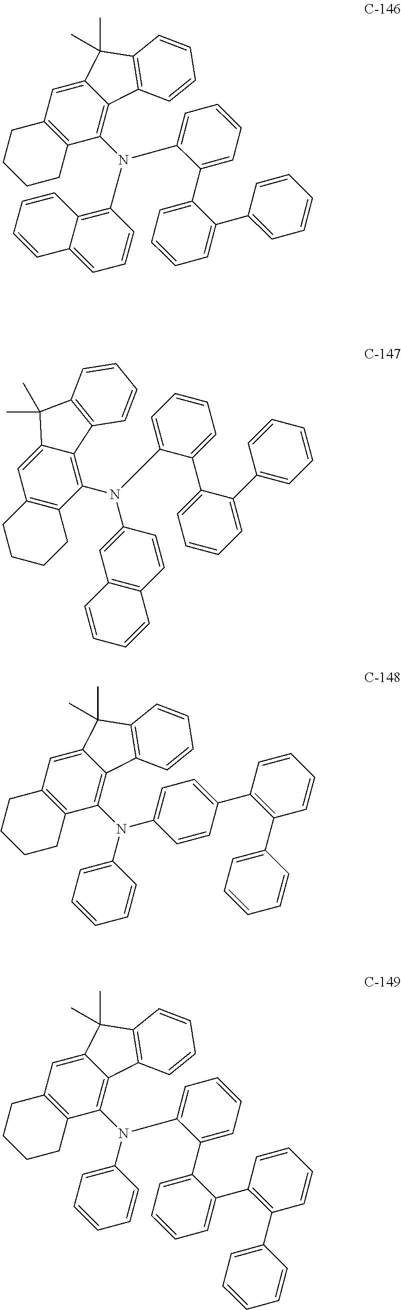 [SMILES: CC1(C)c2ccccc2-c2c1cc1c(c2N(c2ccccc2-c2ccccc2-c2ccccc2)c2cccc3ccccc23)CCCC1 ; CC1(C)c2ccccc2-c2c1cc1c(c2N(c2ccc3ccccc3c2)c2ccccc2-c2ccccc2-c2ccccc2)CCCC1 ; CC1(C)c2ccccc2-c2c1cc1c(c2N(c2ccccc2)c2ccc(-c3ccccc3-c3ccccc3)cc2)CCCC1 ; CC1(C)c2ccccc2-c2c1cc1c(c2N(c2ccccc2)c2ccccc2-c2ccccc2-c2ccccc2-c2ccccc2)CCCC1]

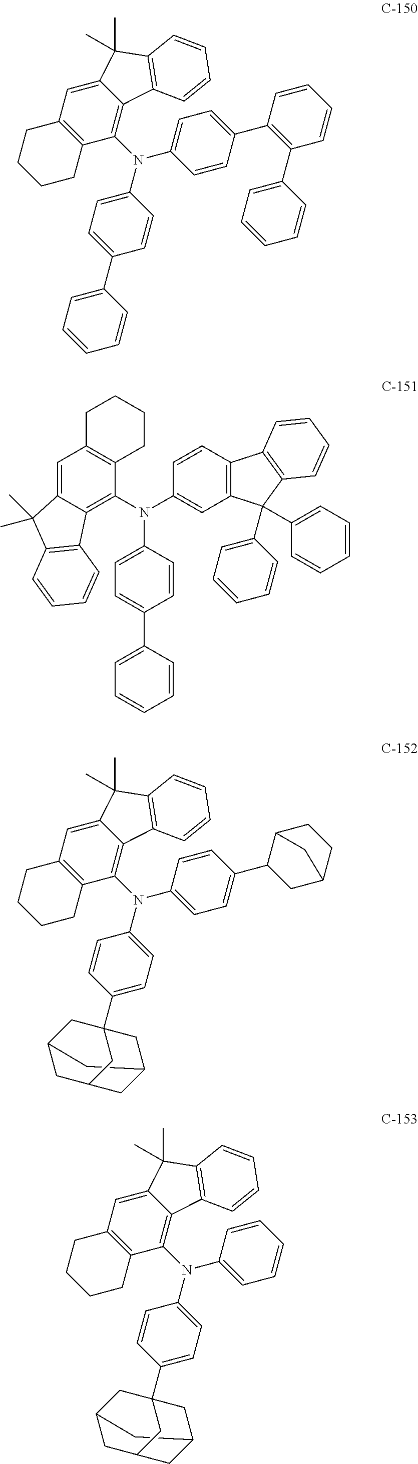 [SMILES: CC1(C)c2ccccc2-c2c1cc1c(c2N(c2ccc(-c3ccccc3)cc2)c2ccc(-c3ccccc3-c3ccccc3)cc2)CCCC1 ; CC1(C)c2ccccc2-c2c1cc1c(c2N(c2ccc(-c3ccccc3)cc2)c2ccc3c(c2)C(c2ccccc2)(c2ccccc2)c2ccccc2-3)CCCC1 ; CC1(C)c2ccccc2-c2c1cc1c(c2N(c2ccc(C3CC4CCC3C4)cc2)c2ccc(C34CC5CC(CC(C5)C3)C4)cc2)CCCC1 ; CC1(C)c2ccccc2-c2c1cc1c(c2N(c2ccccc2)c2ccc(C34CC5CC(CC(C5)C3)C4)cc2)CCCC1]

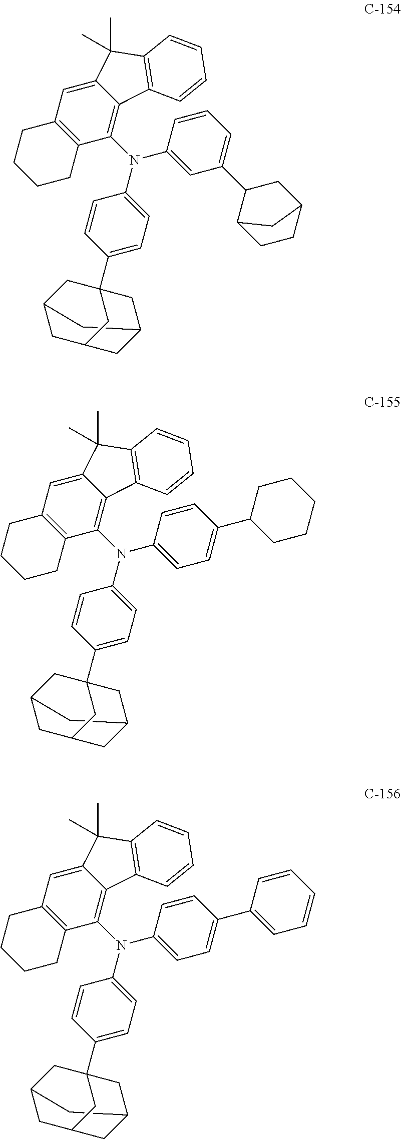 [SMILES: CC1(C)c2ccccc2-c2c1cc1c(c2N(c2ccc(C34CC5CC(CC(C5)C3)C4)cc2)c2cccc(C3CC4CCC3C4)c2)CCCC1 ; CC1(C)c2ccccc2-c2c1cc1c(c2N(c2ccc(C3CCCCC3)cc2)c2ccc(C34CC5CC(CC(C5)C3)C4)cc2)CCCC1 ; CC1(C)c2ccccc2-c2c1cc1c(c2N(c2ccc(-c3ccccc3)cc2)c2ccc(C34CC5CC(CC(C5)C3)C4)cc2)CCCC1]

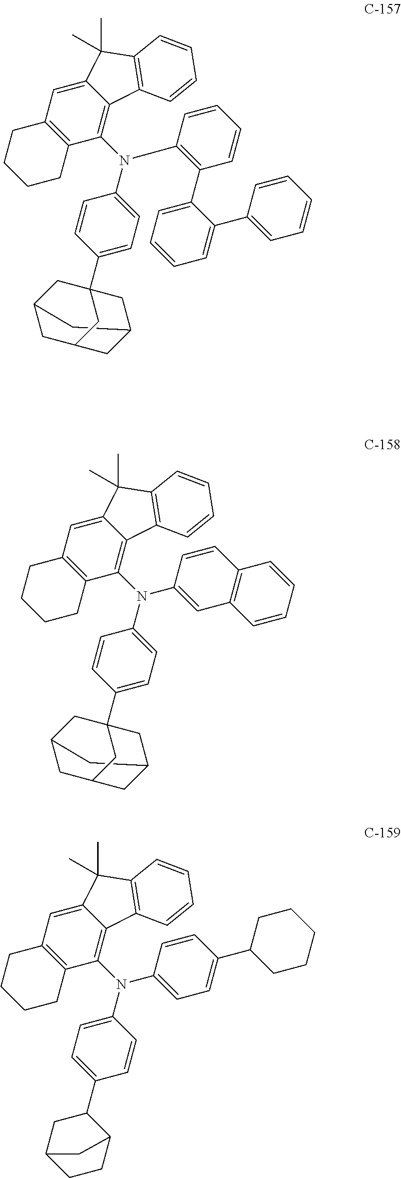 [SMILES: CC1(C)c2ccccc2-c2c1cc1c(c2N(c2ccc(C34CC5CC(CC(C5)C3)C4)cc2)c2ccccc2-c2ccccc2-c2ccccc2)CCCC1 ; CC1(C)c2ccccc2-c2c1cc1c(c2N(c2ccc(C34CC5CC(CC(C5)C3)C4)cc2)c2ccc3ccccc3c2)CCCC1 ; CC1(C)c2ccccc2-c2c1cc1c(c2N(c2ccc(C3CCCCC3)cc2)c2ccc(C3CC4CCC3C4)cc2)CCCC1]

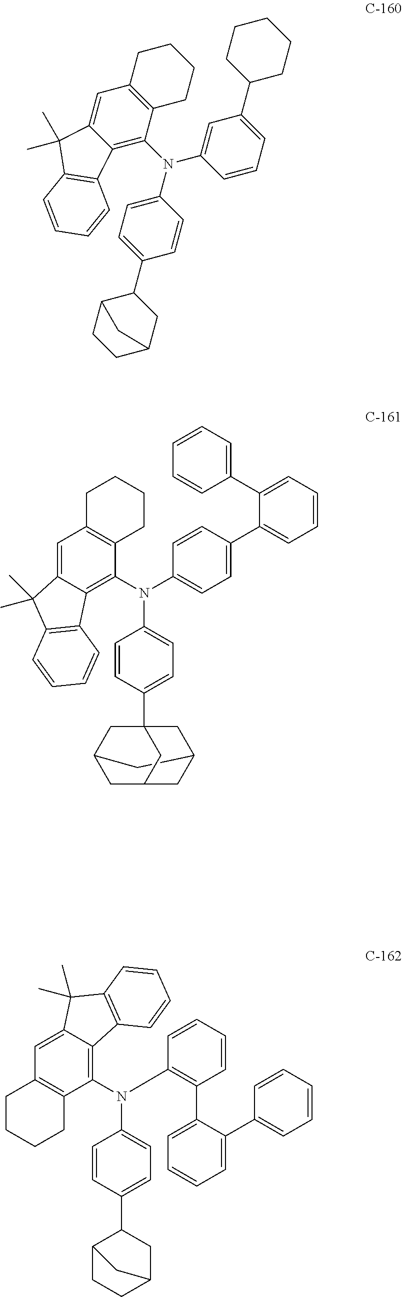 [SMILES: CC1(C)c2ccccc2-c2c1cc1c(c2N(c2ccc(C3CC4CCC3C4)cc2)c2cccc(C3CCCCC3)c2)CCCC1 ; CC1(C)c2ccccc2-c2c1cc1c(c2N(c2ccc(-c3ccccc3-c3ccccc3)cc2)c2ccc(C34CC5CC(CC(C5)C3)C4)cc2)CCCC1 ; CC1(C)c2ccccc2-c2c1cc1c(c2N(c2ccc(C3CC4CCC3C4)cc2)c2ccccc2-c2ccccc2-c2ccccc2)CCCC1]

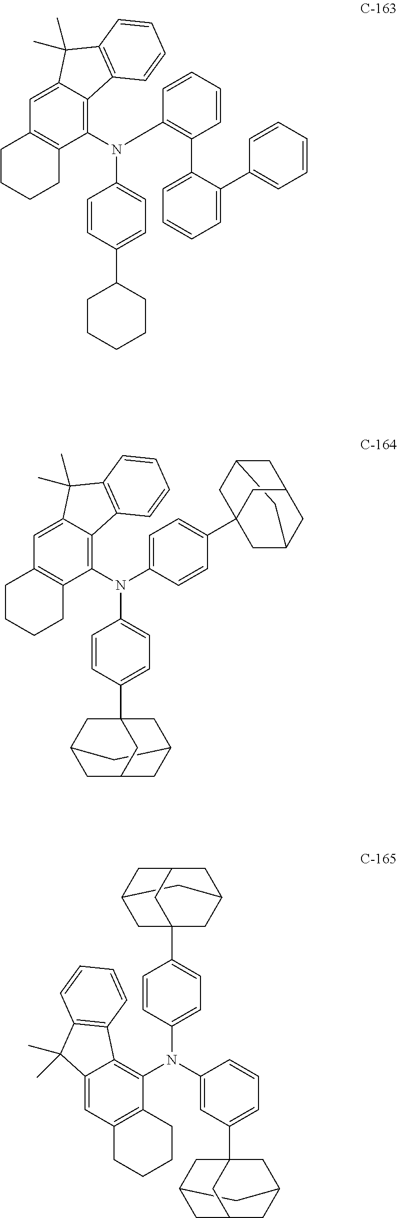 [SMILES: CC1(C)c2ccccc2-c2c1cc1c(c2N(c2ccc(C3CCCCC3)cc2)c2ccccc2-c2ccccc2-c2ccccc2)CCCC1 ; CC1(C)c2ccccc2-c2c1cc1c(c2N(c2ccc(C34CC5CC(CC(C5)C3)C4)cc2)c2ccc(C34CC5CC(CC(C5)C3)C4)cc2)CCCC1 ; CC1(C)c2ccccc2-c2c1cc1c(c2N(c2ccc(C34CC5CC(CC(C5)C3)C4)cc2)c2cccc(C34CC5CC(CC(C5)C3)C4)c2)CCCC1]

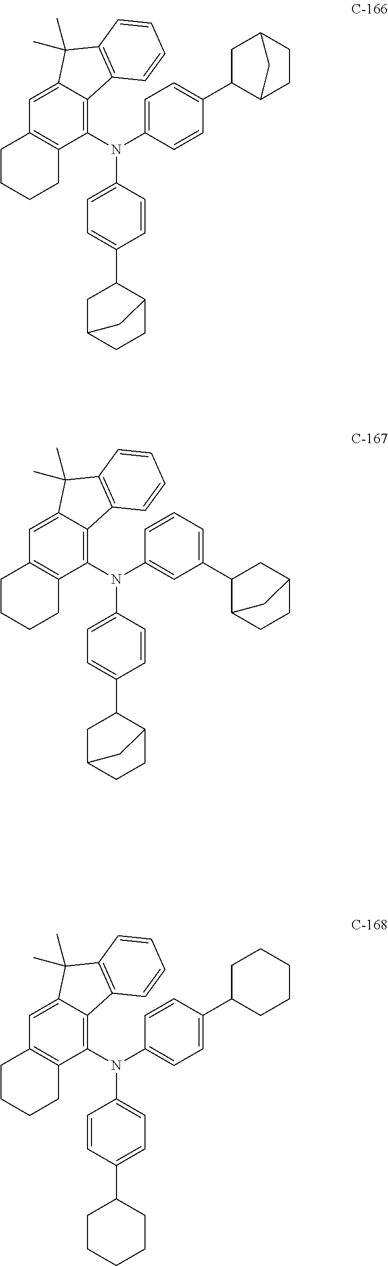 [SMILES: CC1(C)c2ccccc2-c2c1cc1c(c2N(c2ccc(C3CC4CCC3C4)cc2)c2ccc(C3CC4CCC3C4)cc2)CCCC1 ; CC1(C)c2ccccc2-c2c1cc1c(c2N(c2ccc(C3CC4CCC3C4)cc2)c2cccc(C3CC4CCC3C4)c2)CCCC1 ; CC1(C)c2ccccc2-c2c1cc1c(c2N(c2ccc(C3CCCCC3)cc2)c2ccc(C3CCCCC3)cc2)CCCC1]

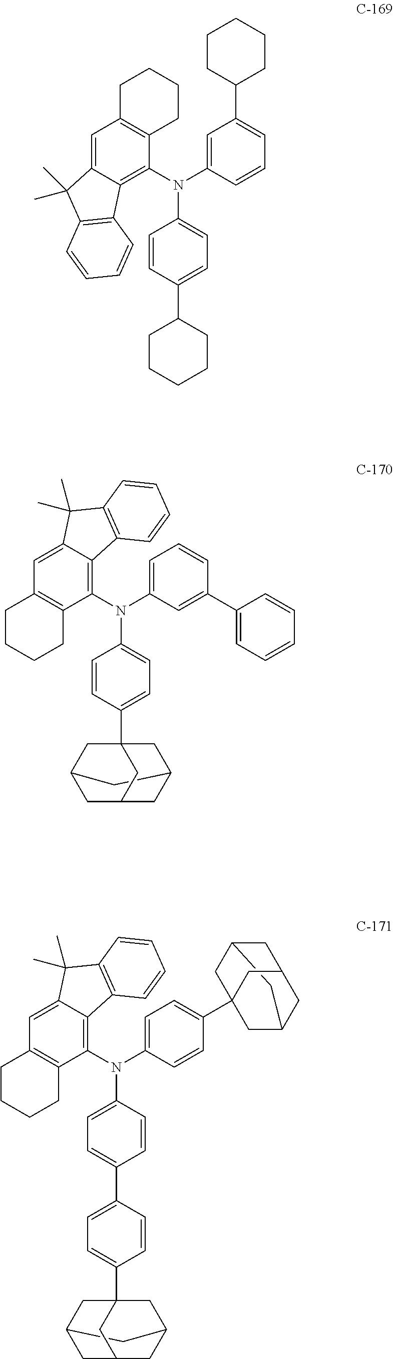 [SMILES: CC1(C)c2ccccc2-c2c1cc1c(c2N(c2ccc(C3CCCCC3)cc2)c2cccc(C3CCCCC3)c2)CCCC1 ; CC1(C)c2ccccc2-c2c1cc1c(c2N(c2ccc(C34CC5CC(CC(C5)C3)C4)cc2)c2cccc(-c3ccccc3)c2)CCCC1 ; CC1(C)c2ccccc2-c2c1cc1c(c2N(c2ccc(-c3ccc(C45CC6CC(CC(C6)C4)C5)cc3)cc2)c2ccc(C34CC5CC(CC(C5)C3)C4)cc2)CCCC1]

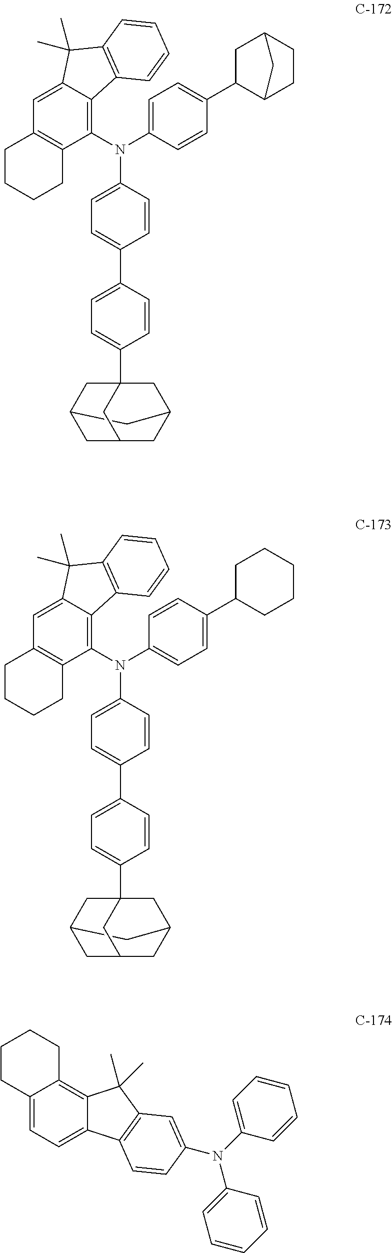 [SMILES: CC1(C)c2ccccc2-c2c1cc1c(c2N(c2ccc(-c3ccc(C45CC6CC(CC(C6)C4)C5)cc3)cc2)c2ccc(C3CC4CCC3C4)cc2)CCCC1 ; CC1(C)c2ccccc2-c2c1cc1c(c2N(c2ccc(-c3ccc(C45CC6CC(CC(C6)C4)C5)cc3)cc2)c2ccc(C3CCCCC3)cc2)CCCC1 ; CC1(C)c2cc(N(c3ccccc3)c3ccccc3)ccc2-c2ccc3c(c21)CCCC3]

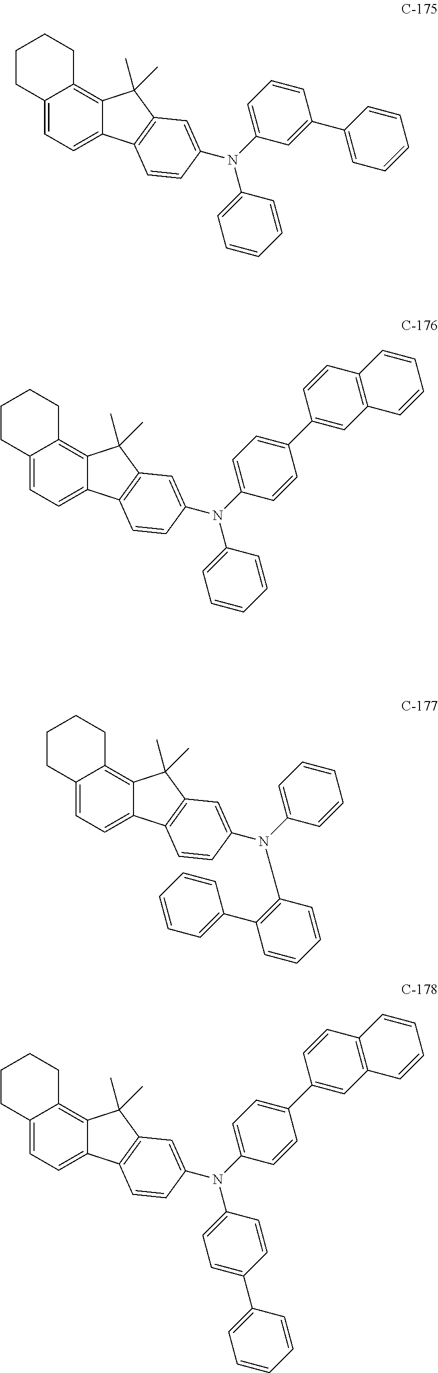 [SMILES: CC1(C)c2cc(N(c3ccccc3)c3cccc(-c4ccccc4)c3)ccc2-c2ccc3c(c21)CCCC3 ; CC1(C)c2cc(N(c3ccccc3)c3ccc(-c4ccc5ccccc5c4)cc3)ccc2-c2ccc3c(c21)CCCC3 ; CC1(C)c2cc(N(c3ccccc3)c3ccccc3-c3ccccc3)ccc2-c2ccc3c(c21)CCCC3 ; CC1(C)c2cc(N(c3ccc(-c4ccccc4)cc3)c3ccc(-c4ccc5ccccc5c4)cc3)ccc2-c2ccc3c(c21)CCCC3]

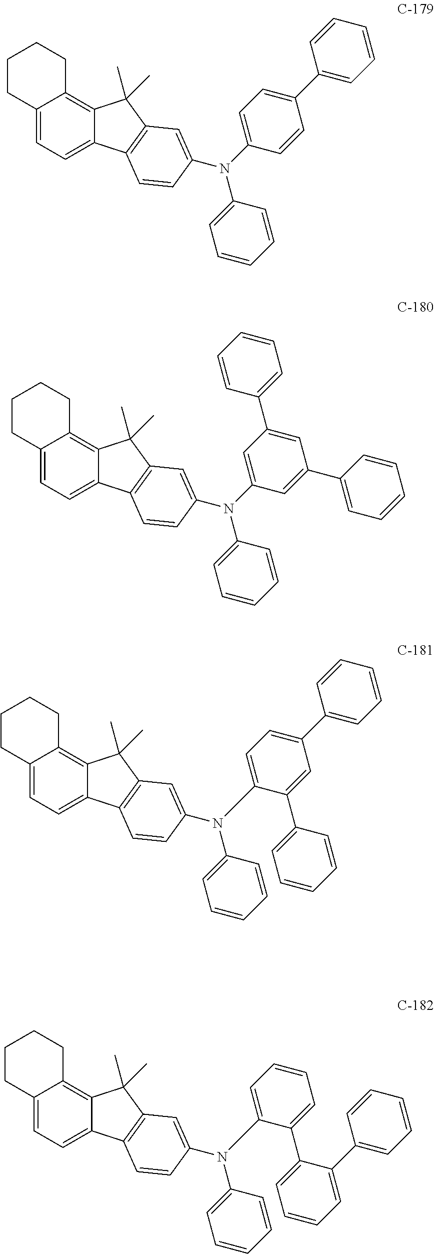 [SMILES: CC1(C)c2cc(N(c3ccccc3)c3ccc(-c4ccccc4)cc3)ccc2-c2ccc3c(c21)CCCC3 ; CC1(C)c2cc(N(c3ccccc3)c3cc(-c4ccccc4)cc(-c4ccccc4)c3)ccc2-c2ccc3c(c21)CCCC3 ; CC1(C)c2cc(N(c3ccccc3)c3ccc(-c4ccccc4)cc3-c3ccccc3)ccc2-c2ccc3c(c21)CCCC3 ; CC1(C)c2cc(N(c3ccccc3)c3ccccc3-c3ccccc3-c3ccccc3)ccc2-c2ccc3c(c21)CCCC3]

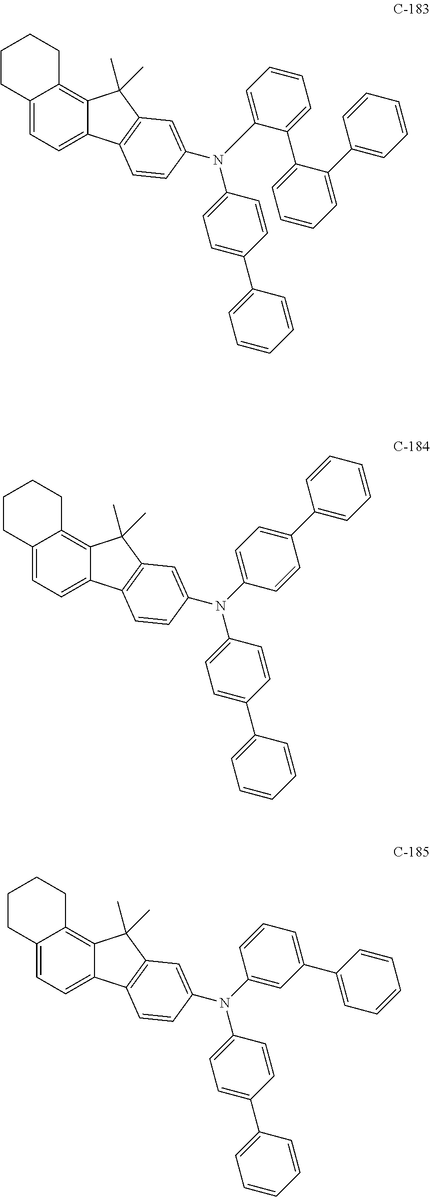 [SMILES: CC1(C)c2cc(N(c3ccc(-c4ccccc4)cc3)c3ccccc3-c3ccccc3-c3ccccc3)ccc2-c2ccc3c(c21)CCCC3 ; CC1(C)c2cc(N(c3ccc(-c4ccccc4)cc3)c3ccc(-c4ccccc4)cc3)ccc2-c2ccc3c(c21)CCCC3 ; CC1(C)c2cc(N(c3ccc(-c4ccccc4)cc3)c3cccc(-c4ccccc4)c3)ccc2-c2ccc3c(c21)CCCC3]

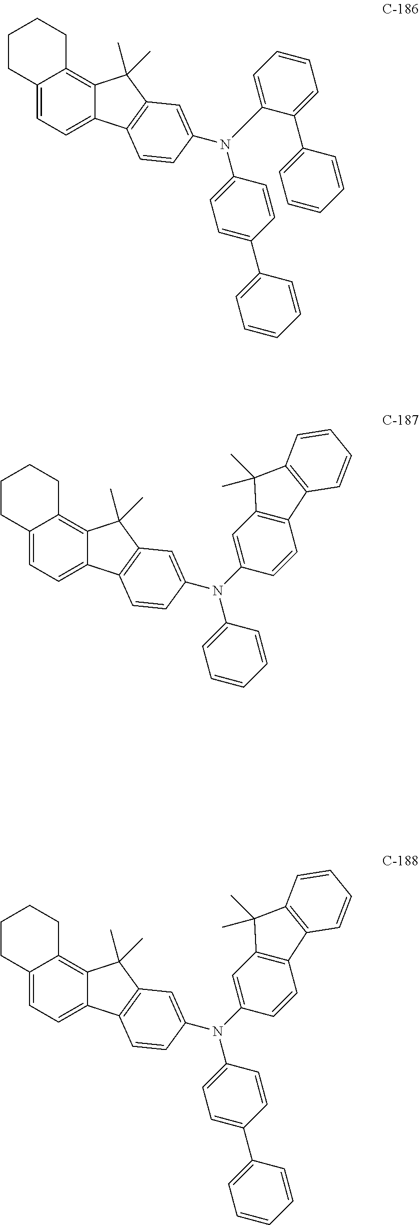 [SMILES: CC1(C)c2cc(N(c3ccc(-c4ccccc4)cc3)c3ccccc3-c3ccccc3)ccc2-c2ccc3c(c21)CCCC3 ; CC1(C)c2ccccc2-c2ccc(N(c3ccccc3)c3ccc4c(c3)C(C)(C)c3c-4ccc4c3CCCC4)cc21 ; CC1(C)c2ccccc2-c2ccc(N(c3ccc(-c4ccccc4)cc3)c3ccc4c(c3)C(C)(C)c3c-4ccc4c3CCCC4)cc21]

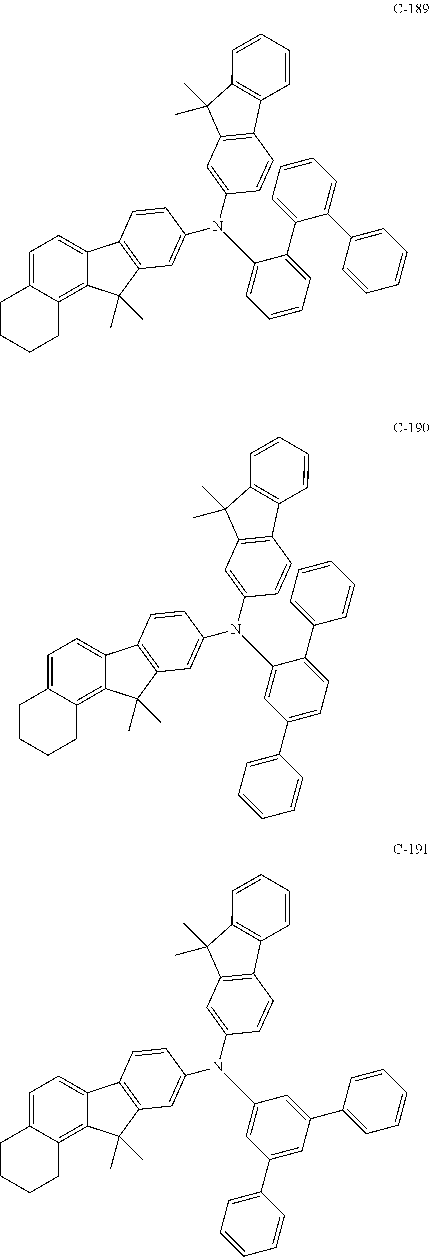 [SMILES: CC1(C)c2ccccc2-c2ccc(N(c3ccc4c(c3)C(C)(C)c3c-4ccc4c3CCCC4)c3ccccc3-c3ccccc3-c3ccccc3)cc21 ; CC1(C)c2ccccc2-c2ccc(N(c3ccc4c(c3)C(C)(C)c3c-4ccc4c3CCCC4)c3cc(-c4ccccc4)ccc3-c3ccccc3)cc21 ; CC1(C)c2ccccc2-c2ccc(N(c3cc(-c4ccccc4)cc(-c4ccccc4)c3)c3ccc4c(c3)C(C)(C)c3c-4ccc4c3CCCC4)cc21]

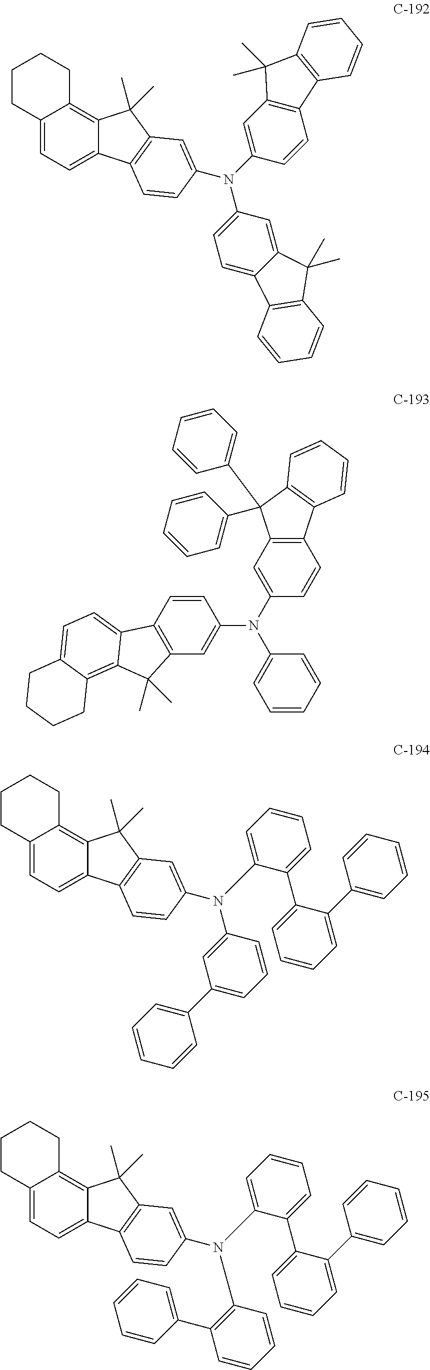 [SMILES: CC1(C)c2ccccc2-c2ccc(N(c3ccc4c(c3)C(C)(C)c3ccccc3-4)c3ccc4c(c3)C(C)(C)c3c-4ccc4c3CCCC4)cc21 ; CC1(C)c2cc(N(c3ccccc3)c3ccc4c(c3)C(c3ccccc3)(c3ccccc3)c3ccccc3-4)ccc2-c2ccc3c(c21)CCCC3 ; CC1(C)c2cc(N(c3cccc(-c4ccccc4)c3)c3ccccc3-c3ccccc3-c3ccccc3)ccc2-c2ccc3c(c21)CCCC3 ; CC1(C)c2cc(N(c3ccccc3-c3ccccc3)c3ccccc3-c3ccccc3-c3ccccc3)ccc2-c2ccc3c(c21)CCCC3]

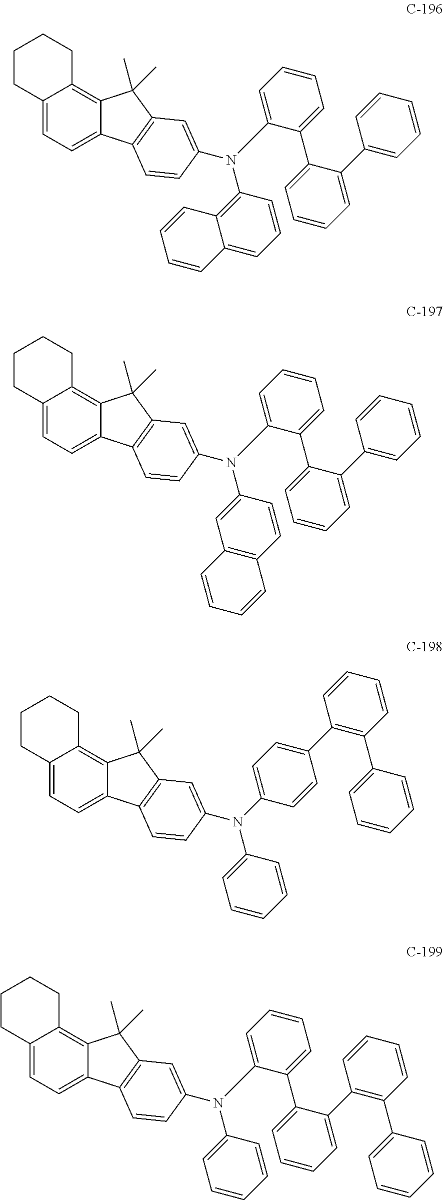 [SMILES: CC1(C)c2cc(N(c3ccccc3-c3ccccc3-c3ccccc3)c3cccc4ccccc34)ccc2-c2ccc3c(c21)CCCC3 ; CC1(C)c2cc(N(c3ccc4ccccc4c3)c3ccccc3-c3ccccc3-c3ccccc3)ccc2-c2ccc3c(c21)CCCC3 ; CC1(C)c2cc(N(c3ccccc3)c3ccc(-c4ccccc4-c4ccccc4)cc3)ccc2-c2ccc3c(c21)CCCC3 ; CC1(C)c2cc(N(c3ccccc3)c3ccccc3-c3ccccc3-c3ccccc3-c3ccccc3)ccc2-c2ccc3c(c21)CCCC3]

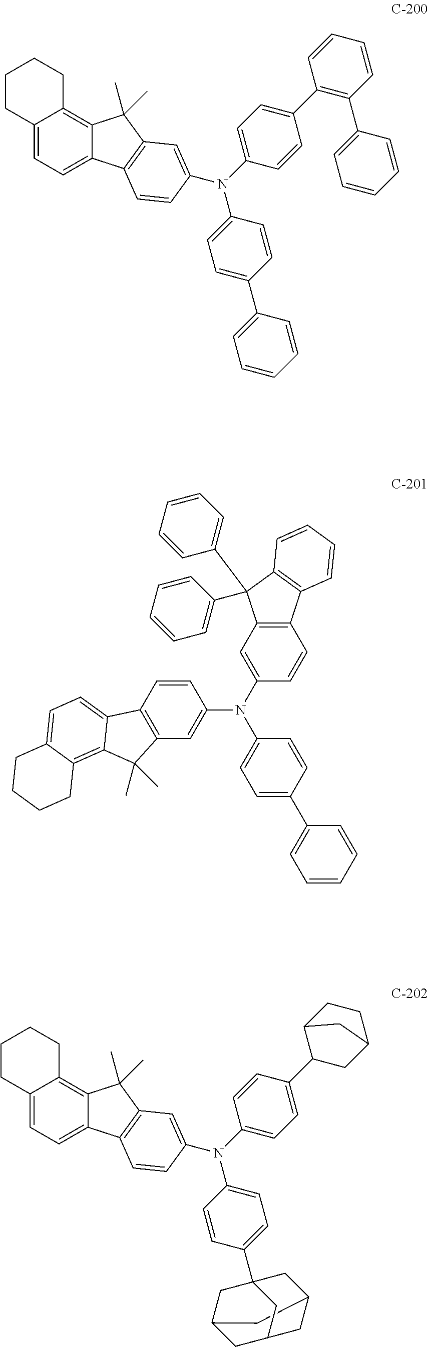 [SMILES: CC1(C)c2cc(N(c3ccc(-c4ccccc4)cc3)c3ccc(-c4ccccc4-c4ccccc4)cc3)ccc2-c2ccc3c(c21)CCCC3 ; CC1(C)c2cc(N(c3ccc(-c4ccccc4)cc3)c3ccc4c(c3)C(c3ccccc3)(c3ccccc3)c3ccccc3-4)ccc2-c2ccc3c(c21)CCCC3 ; CC1(C)c2cc(N(c3ccc(C4CC5CCC4C5)cc3)c3ccc(C45CC6CC(CC(C6)C4)C5)cc3)ccc2-c2ccc3c(c21)CCCC3]

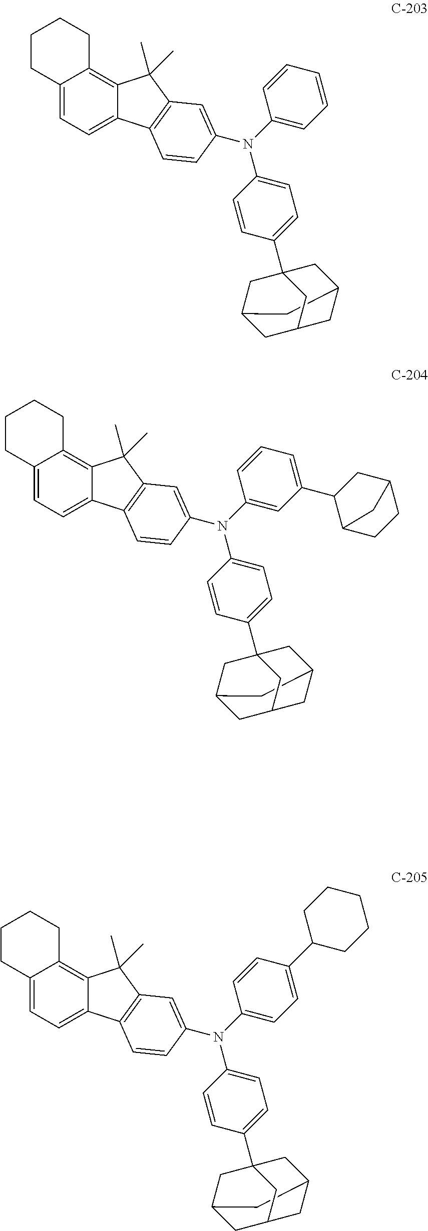 [SMILES: CC1(C)c2cc(N(c3ccccc3)c3ccc(C45CC6CC(CC(C6)C4)C5)cc3)ccc2-c2ccc3c(c21)CCCC3 ; CC1(C)c2cc(N(c3ccc(C45CC6CC(CC(C6)C4)C5)cc3)c3cccc(C4CC5CCC4C5)c3)ccc2-c2ccc3c(c21)CCCC3 ; CC1(C)c2cc(N(c3ccc(C4CCCCC4)cc3)c3ccc(C45CC6CC(CC(C6)C4)C5)cc3)ccc2-c2ccc3c(c21)CCCC3]

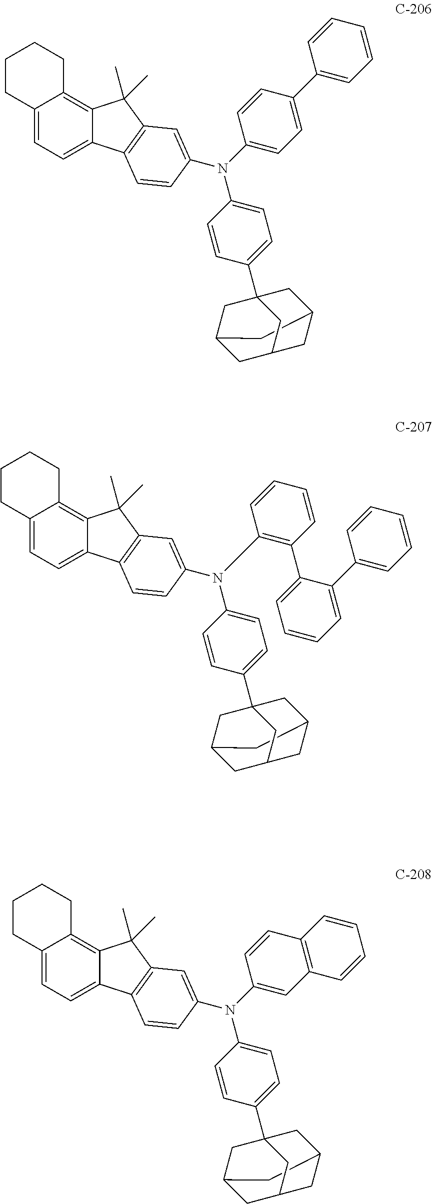 [SMILES: CC1(C)c2cc(N(c3ccc(-c4ccccc4)cc3)c3ccc(C45CC6CC(CC(C6)C4)C5)cc3)ccc2-c2ccc3c(c21)CCCC3 ; CC1(C)c2cc(N(c3ccc(C45CC6CC(CC(C6)C4)C5)cc3)c3ccccc3-c3ccccc3-c3ccccc3)ccc2-c2ccc3c(c21)CCCC3 ; CC1(C)c2cc(N(c3ccc(C45CC6CC(CC(C6)C4)C5)cc3)c3ccc4ccccc4c3)ccc2-c2ccc3c(c21)CCCC3]

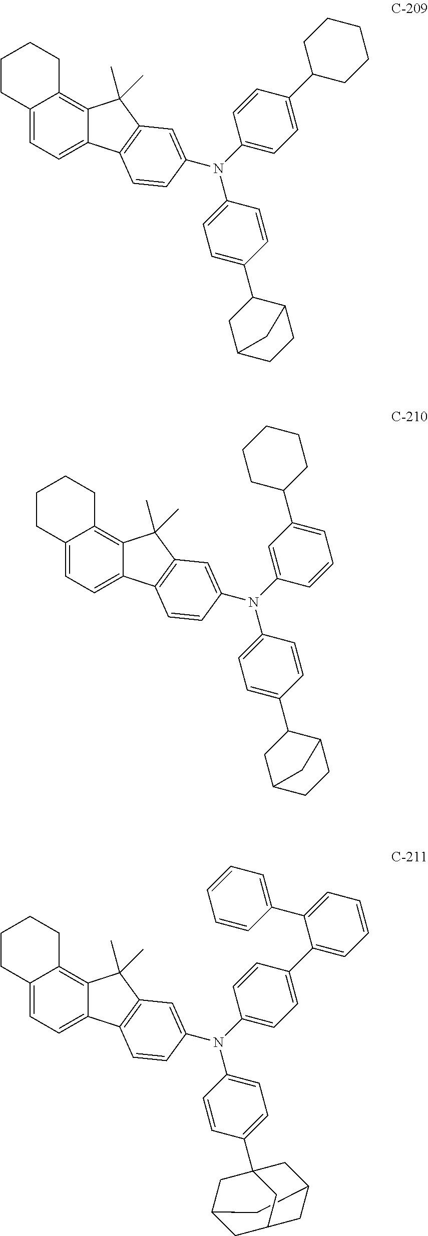 [SMILES: CC1(C)c2cc(N(c3ccc(C4CCCCC4)cc3)c3ccc(C4CC5CCC4C5)cc3)ccc2-c2ccc3c(c21)CCCC3 ; CC1(C)c2cc(N(c3ccc(C4CC5CCC4C5)cc3)c3cccc(C4CCCCC4)c3)ccc2-c2ccc3c(c21)CCCC3 ; CC1(C)c2cc(N(c3ccc(-c4ccccc4-c4ccccc4)cc3)c3ccc(C45CC6CC(CC(C6)C4)C5)cc3)ccc2-c2ccc3c(c21)CCCC3]

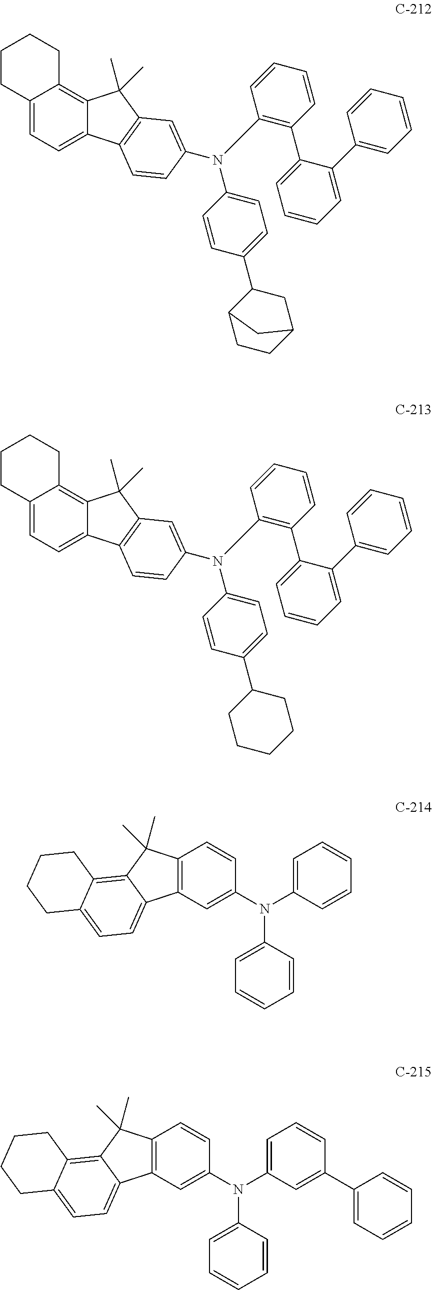 [SMILES: CC1(C)c2cc(N(c3ccc(C4CC5CCC4C5)cc3)c3ccccc3-c3ccccc3-c3ccccc3)ccc2-c2ccc3c(c21)CCCC3 ; CC1(C)c2cc(N(c3ccc(C4CCCCC4)cc3)c3ccccc3-c3ccccc3-c3ccccc3)ccc2-c2ccc3c(c21)CCCC3 ; CC1(C)c2ccc(N(c3ccccc3)c3ccccc3)cc2-c2ccc3c(c21)CCCC3 ; CC1(C)c2ccc(N(c3ccccc3)c3cccc(-c4ccccc4)c3)cc2-c2ccc3c(c21)CCCC3]

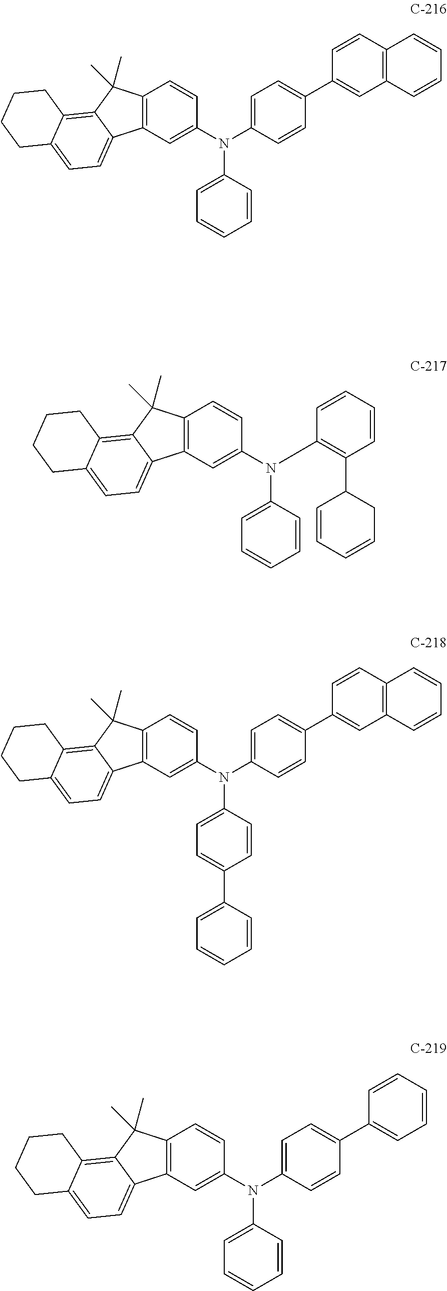 [SMILES: CC1(C)c2ccc(N(c3ccccc3)c3ccc(-c4ccc5ccccc5c4)cc3)cc2-c2ccc3c(c21)CCCC3 ; CC1(C)c2ccc(N(c3ccccc3)c3ccccc3C3C=CC=CC3)cc2-c2ccc3c(c21)CCCC3 ; CC1(C)c2ccc(N(c3ccc(-c4ccccc4)cc3)c3ccc(-c4ccc5ccccc5c4)cc3)cc2-c2ccc3c(c21)CCCC3 ; CC1(C)c2ccc(N(c3ccccc3)c3ccc(-c4ccccc4)cc3)cc2-c2ccc3c(c21)CCCC3]

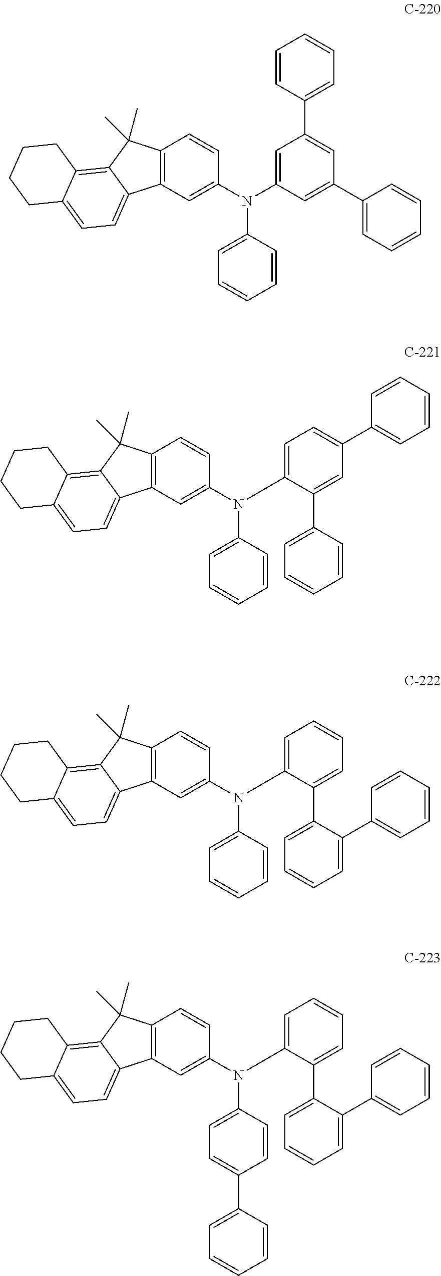 [SMILES: CC1(C)c2ccc(N(c3ccccc3)c3cc(-c4ccccc4)cc(-c4ccccc4)c3)cc2-c2ccc3c(c21)CCCC3 ; CC1(C)c2ccc(N(c3ccccc3)c3ccc(-c4ccccc4)cc3-c3ccccc3)cc2-c2ccc3c(c21)CCCC3 ; CC1(C)c2ccc(N(c3ccccc3)c3ccccc3-c3ccccc3-c3ccccc3)cc2-c2ccc3c(c21)CCCC3 ; CC1(C)c2ccc(N(c3ccc(-c4ccccc4)cc3)c3ccccc3-c3ccccc3-c3ccccc3)cc2-c2ccc3c(c21)CCCC3]

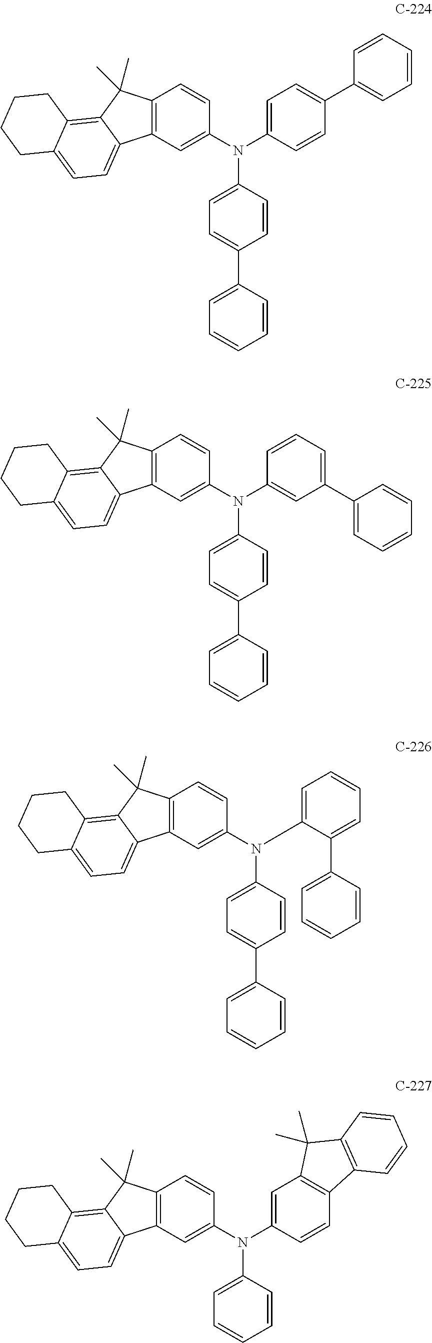 [SMILES: CC1(C)c2ccc(N(c3ccc(-c4ccccc4)cc3)c3ccc(-c4ccccc4)cc3)cc2-c2ccc3c(c21)CCCC3 ; CC1(C)c2ccc(N(c3ccc(-c4ccccc4)cc3)c3cccc(-c4ccccc4)c3)cc2-c2ccc3c(c21)CCCC3 ; CC1(C)c2ccc(N(c3ccc(-c4ccccc4)cc3)c3ccccc3-c3ccccc3)cc2-c2ccc3c(c21)CCCC3 ; CC1(C)c2ccccc2-c2ccc(N(c3ccccc3)c3ccc4c(c3)-c3ccc5c(c3C4(C)C)CCCC5)cc21]

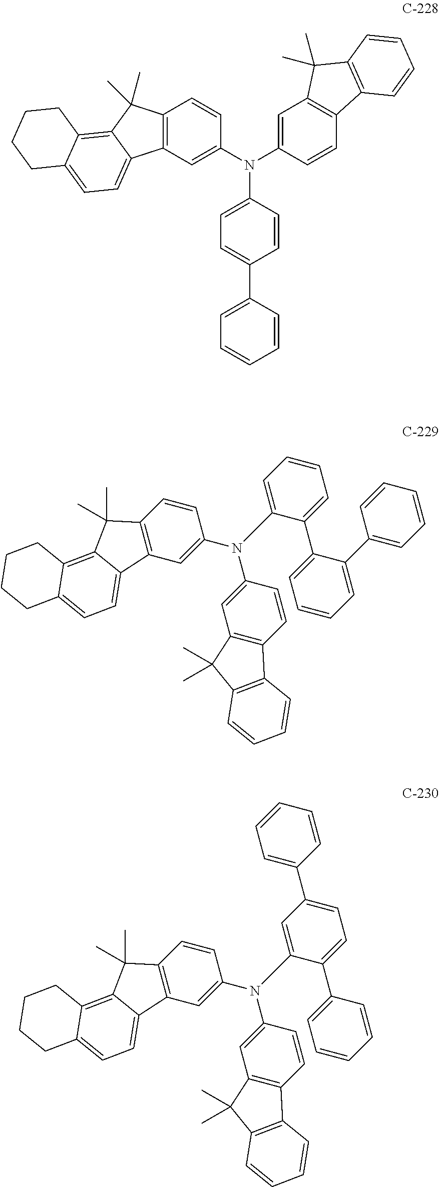 [SMILES: CC1(C)c2ccccc2-c2ccc(N(c3ccc(-c4ccccc4)cc3)c3ccc4c(c3)-c3ccc5c(c3C4(C)C)CCCC5)cc21 ; CC1(C)c2ccccc2-c2ccc(N(c3ccc4c(c3)-c3ccc5c(c3C4(C)C)CCCC5)c3ccccc3-c3ccccc3-c3ccccc3)cc21 ; CC1(C)c2ccccc2-c2ccc(N(c3ccc4c(c3)-c3ccc5c(c3C4(C)C)CCCC5)c3cc(-c4ccccc4)ccc3-c3ccccc3)cc21]

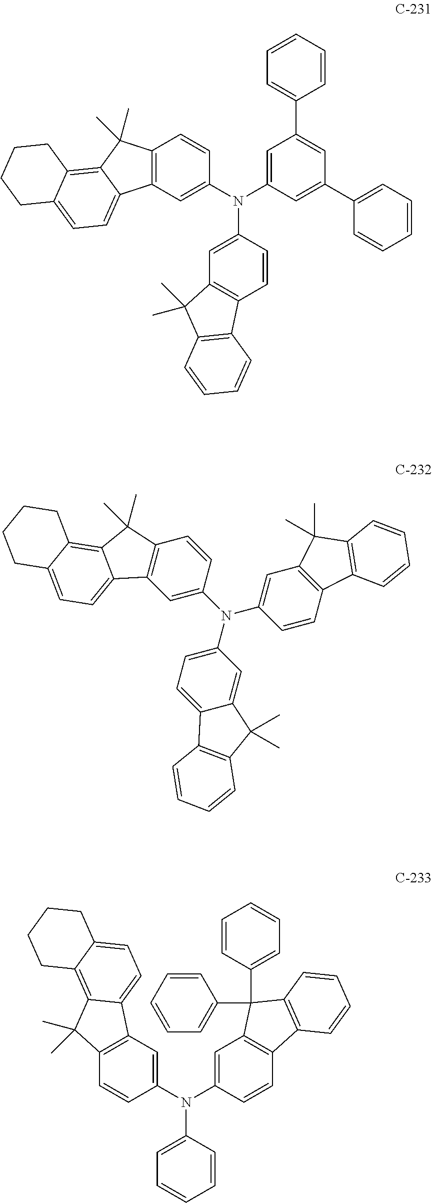 [SMILES: CC1(C)c2ccccc2-c2ccc(N(c3cc(-c4ccccc4)cc(-c4ccccc4)c3)c3ccc4c(c3)-c3ccc5c(c3C4(C)C)CCCC5)cc21 ; CC1(C)c2ccccc2-c2ccc(N(c3ccc4c(c3)-c3ccc5c(c3C4(C)C)CCCC5)c3ccc4c(c3)C(C)(C)c3ccccc3-4)cc21 ; CC1(C)c2ccc(N(c3ccccc3)c3ccc4c(c3)C(c3ccccc3)(c3ccccc3)c3ccccc3-4)cc2-c2ccc3c(c21)CCCC3]

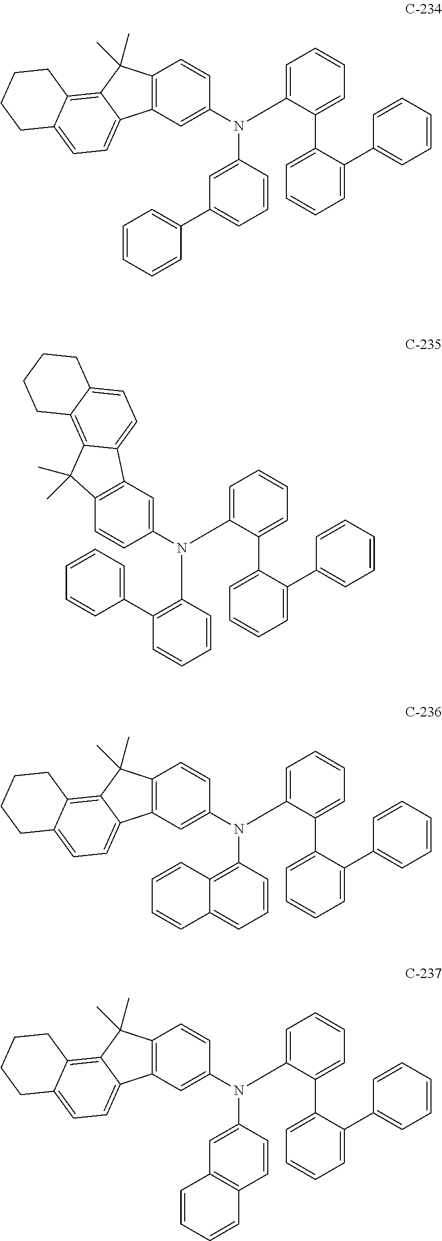 [SMILES: CC1(C)c2ccc(N(c3cccc(-c4ccccc4)c3)c3ccccc3-c3ccccc3-c3ccccc3)cc2-c2ccc3c(c21)CCCC3 ; CC1(C)c2ccc(N(c3ccccc3-c3ccccc3)c3ccccc3-c3ccccc3-c3ccccc3)cc2-c2ccc3c(c21)CCCC3 ; CC1(C)c2ccc(N(c3ccccc3-c3ccccc3-c3ccccc3)c3cccc4ccccc34)cc2-c2ccc3c(c21)CCCC3 ; CC1(C)c2ccc(N(c3ccc4ccccc4c3)c3ccccc3-c3ccccc3-c3ccccc3)cc2-c2ccc3c(c21)CCCC3]

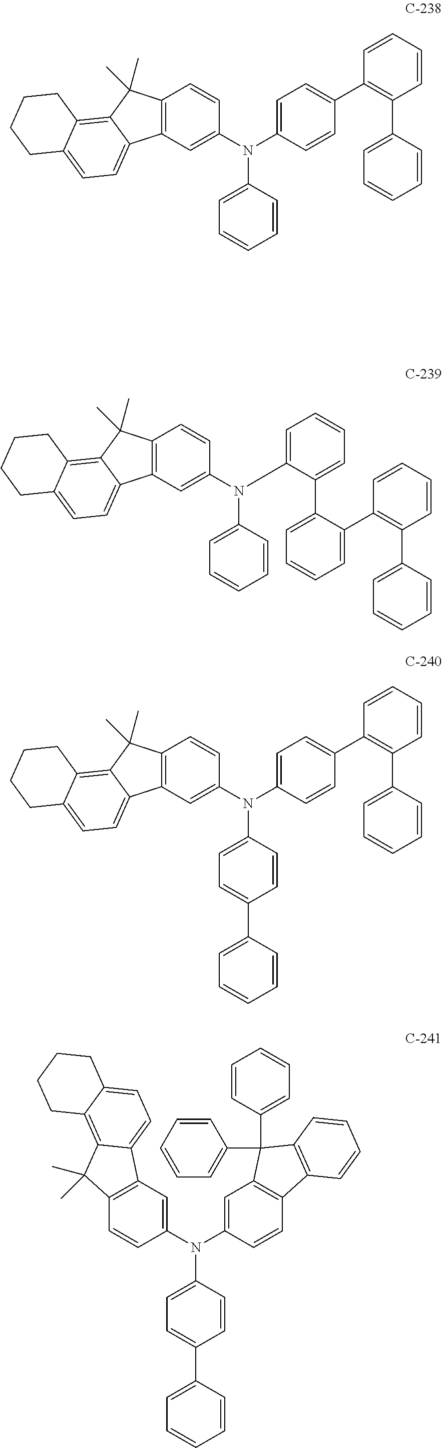

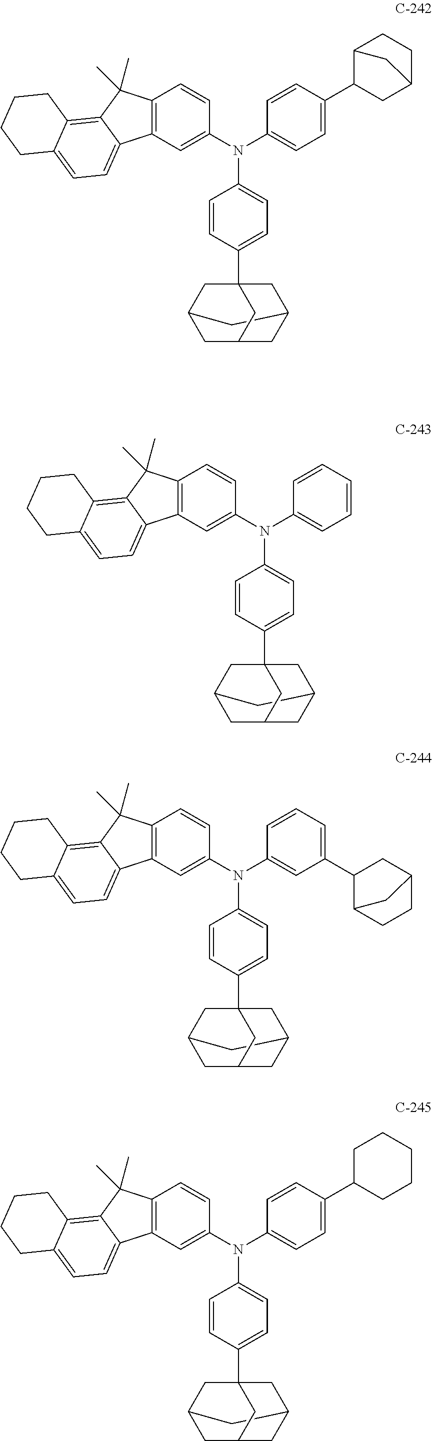 [SMILES: CC1(C)c2ccc(N(c3ccc(C4CC5CCC4C5)cc3)c3ccc(C45CC6CC(CC(C6)C4)C5)cc3)cc2-c2ccc3c(c21)CCCC3 ; CC1(C)c2ccc(N(c3ccccc3)c3ccc(C45CC6CC(CC(C6)C4)C5)cc3)cc2-c2ccc3c(c21)CCCC3 ; CC1(C)c2ccc(N(c3ccc(C45CC6CC(CC(C6)C4)C5)cc3)c3cccc(C4CC5CCC4C5)c3)cc2-c2ccc3c(c21)CCCC3 ; CC1(C)c2ccc(N(c3ccc(C4CCCCC4)cc3)c3ccc(C45CC6CC(CC(C6)C4)C5)cc3)cc2-c2ccc3c(c21)CCCC3]

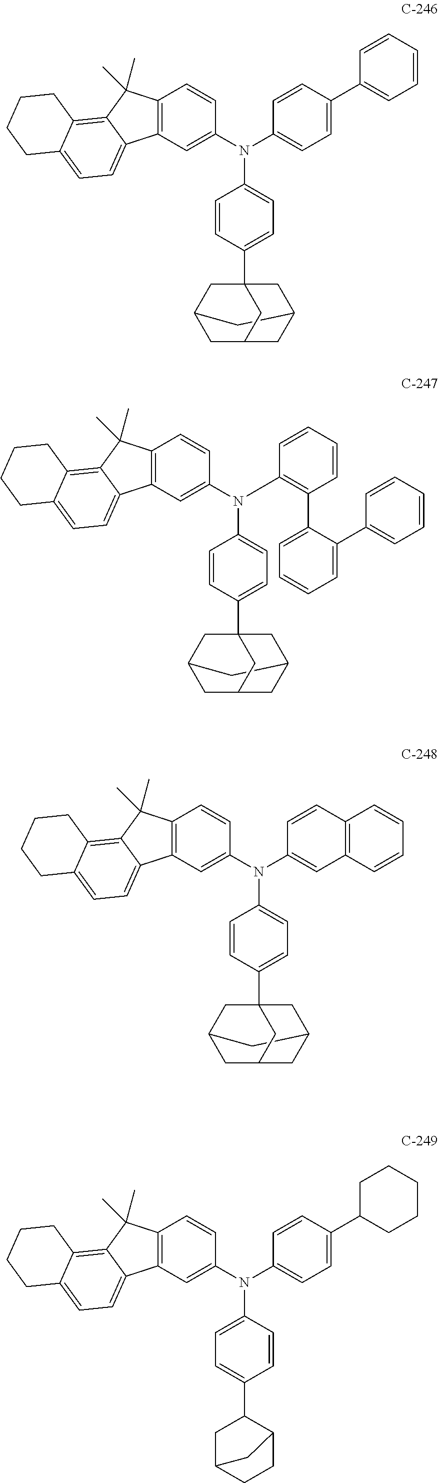 [SMILES: CC1(C)c2ccc(N(c3ccc(-c4ccccc4)cc3)c3ccc(C45CC6CC(CC(C6)C4)C5)cc3)cc2-c2ccc3c(c21)CCCC3 ; CC1(C)c2ccc(N(c3ccc(C45CC6CC(CC(C6)C4)C5)cc3)c3ccccc3-c3ccccc3-c3ccccc3)cc2-c2ccc3c(c21)CCCC3 ; CC1(C)c2ccc(N(c3ccc(C45CC6CC(CC(C6)C4)C5)cc3)c3ccc4ccccc4c3)cc2-c2ccc3c(c21)CCCC3 ; CC1(C)c2ccc(N(c3ccc(C4CCCCC4)cc3)c3ccc(C4CC5CCC4C5)cc3)cc2-c2ccc3c(c21)CCCC3]

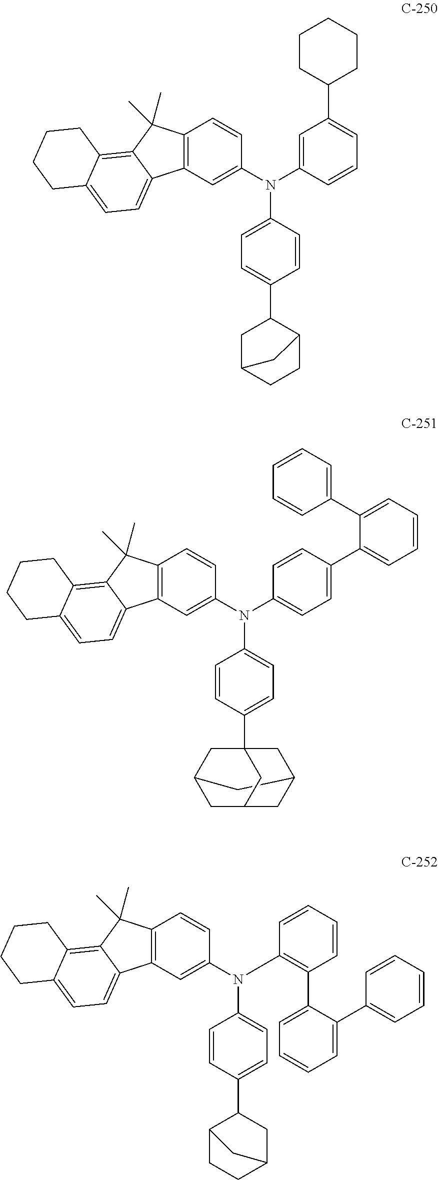 [SMILES: CC1(C)c2ccc(N(c3ccc(C4CC5CCC4C5)cc3)c3cccc(C4CCCCC4)c3)cc2-c2ccc3c(c21)CCCC3 ; CC1(C)c2ccc(N(c3ccc(-c4ccccc4-c4ccccc4)cc3)c3ccc(C45CC6CC(CC(C6)C4)C5)cc3)cc2-c2ccc3c(c21)CCCC3 ; CC1(C)c2ccc(N(c3ccc(C4CC5CCC4C5)cc3)c3ccccc3-c3ccccc3-c3ccccc3)cc2-c2ccc3c(c21)CCCC3]

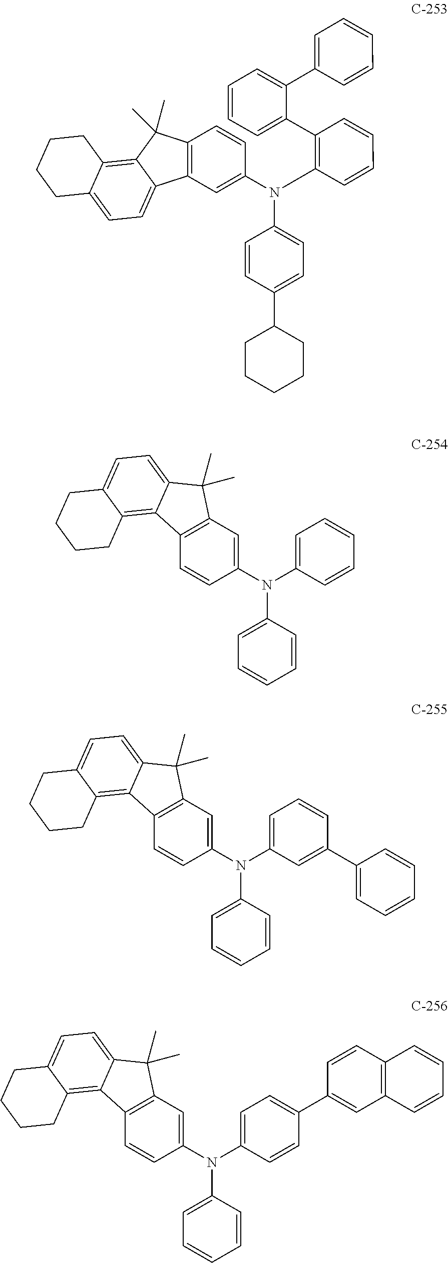 [SMILES: CC1(C)c2ccc(N(c3ccc(C4CCCCC4)cc3)c3ccccc3-c3ccccc3-c3ccccc3)cc2-c2ccc3c(c21)CCCC3 ; CC1(C)c2cc(N(c3ccccc3)c3ccccc3)ccc2-c2c1ccc1c2CCCC1 ; CC1(C)c2cc(N(c3ccccc3)c3cccc(-c4ccccc4)c3)ccc2-c2c1ccc1c2CCCC1 ; CC1(C)c2cc(N(c3ccccc3)c3ccc(-c4ccc5ccccc5c4)cc3)ccc2-c2c1ccc1c2CCCC1]

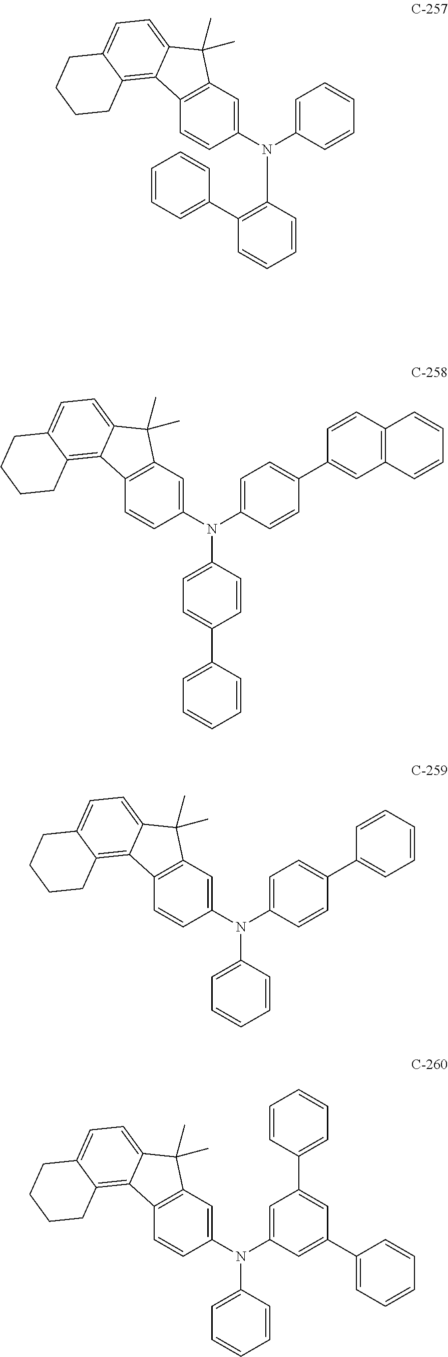 [SMILES: CC1(C)c2cc(N(c3ccccc3)c3ccccc3-c3ccccc3)ccc2-c2c1ccc1c2CCCC1 ; CC1(C)c2cc(N(c3ccc(-c4ccccc4)cc3)c3ccc(-c4ccc5ccccc5c4)cc3)ccc2-c2c1ccc1c2CCCC1 ; CC1(C)c2cc(N(c3ccccc3)c3ccc(-c4ccccc4)cc3)ccc2-c2c1ccc1c2CCCC1 ; CC1(C)c2cc(N(c3ccccc3)c3cc(-c4ccccc4)cc(-c4ccccc4)c3)ccc2-c2c1ccc1c2CCCC1]

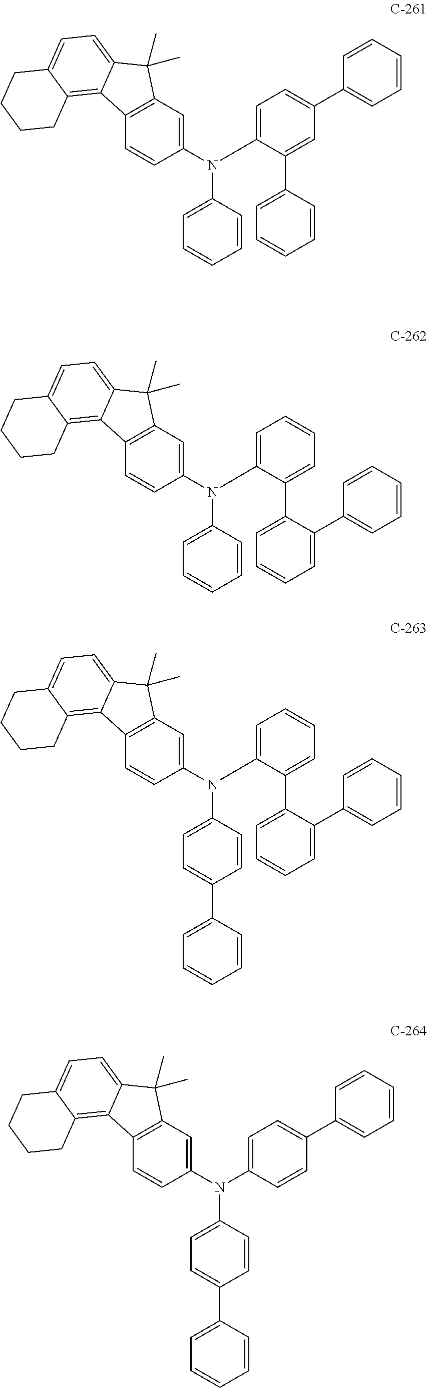 [SMILES: CC1(C)c2cc(N(c3ccccc3)c3ccc(-c4ccccc4)cc3-c3ccccc3)ccc2-c2c1ccc1c2CCCC1 ; CC1(C)c2cc(N(c3ccccc3)c3ccccc3-c3ccccc3-c3ccccc3)ccc2-c2c1ccc1c2CCCC1 ; CC1(C)c2cc(N(c3ccc(-c4ccccc4)cc3)c3ccccc3-c3ccccc3-c3ccccc3)ccc2-c2c1ccc1c2CCCC1 ; CC1(C)c2cc(N(c3ccc(-c4ccccc4)cc3)c3ccc(-c4ccccc4)cc3)ccc2-c2c1ccc1c2CCCC1]

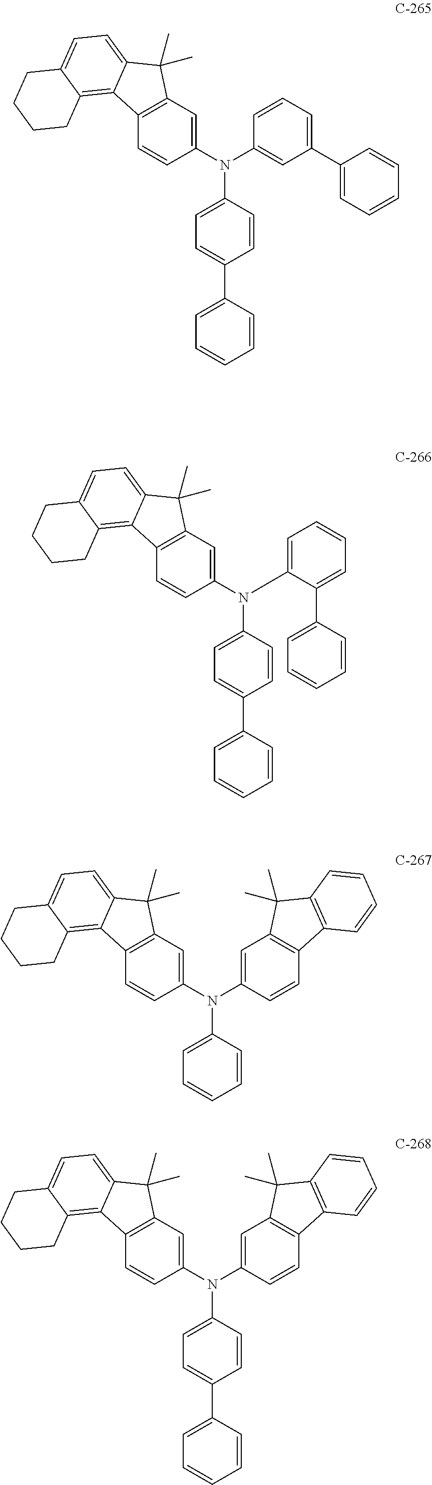 [SMILES: CC1(C)c2cc(N(c3ccc(-c4ccccc4)cc3)c3cccc(-c4ccccc4)c3)ccc2-c2c1ccc1c2CCCC1 ; CC1(C)c2cc(N(c3ccc(-c4ccccc4)cc3)c3ccccc3-c3ccccc3)ccc2-c2c1ccc1c2CCCC1 ; CC1(C)c2ccccc2-c2ccc(N(c3ccccc3)c3ccc4c(c3)C(C)(C)c3ccc5c(c3-4)CCCC5)cc21 ; CC1(C)c2ccccc2-c2ccc(N(c3ccc(-c4ccccc4)cc3)c3ccc4c(c3)C(C)(C)c3ccc5c(c3-4)CCCC5)cc21]

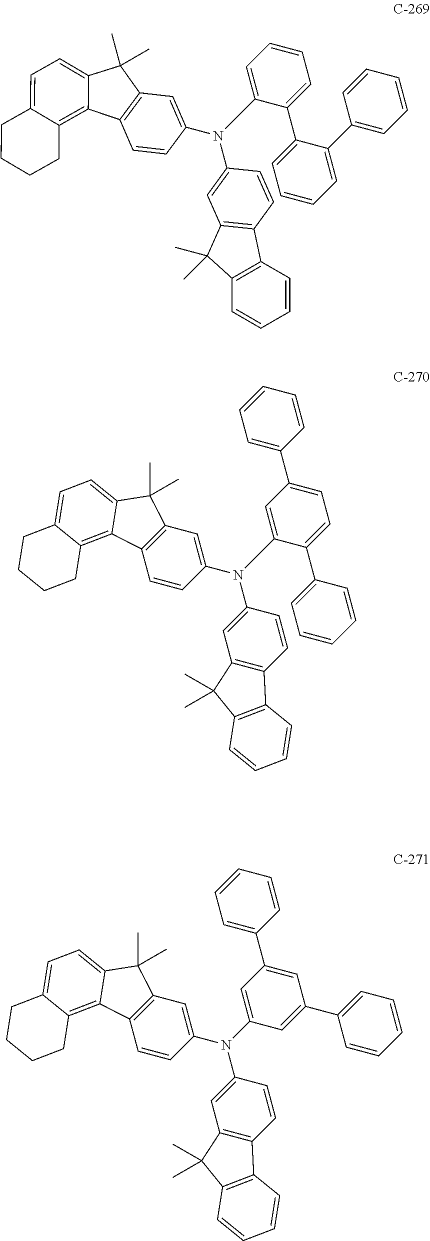 [SMILES: CC1(C)c2ccccc2-c2ccc(N(c3ccc4c(c3)C(C)(C)c3ccc5c(c3-4)CCCC5)c3ccccc3-c3ccccc3-c3ccccc3)cc21 ; CC1(C)c2ccccc2-c2ccc(N(c3ccc4c(c3)C(C)(C)c3ccc5c(c3-4)CCCC5)c3cc(-c4ccccc4)ccc3-c3ccccc3)cc21 ; CC1(C)c2ccccc2-c2ccc(N(c3cc(-c4ccccc4)cc(-c4ccccc4)c3)c3ccc4c(c3)C(C)(C)c3ccc5c(c3-4)CCCC5)cc21]

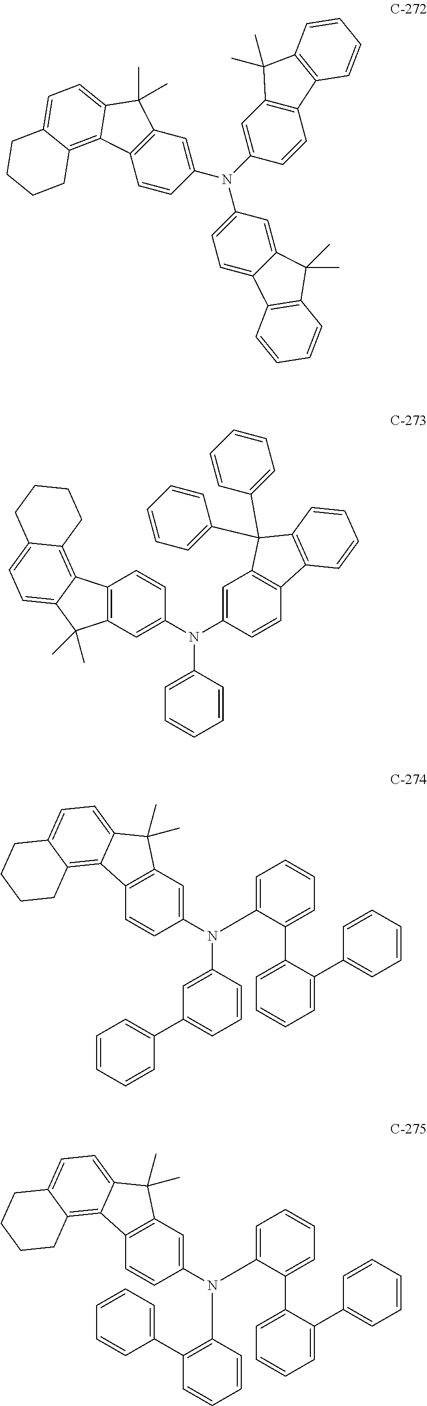 [SMILES: CC1(C)c2ccccc2-c2ccc(N(c3ccc4c(c3)C(C)(C)c3ccccc3-4)c3ccc4c(c3)C(C)(C)c3ccc5c(c3-4)CCCC5)cc21 ; CC1(C)c2cc(N(c3ccccc3)c3ccc4c(c3)C(c3ccccc3)(c3ccccc3)c3ccccc3-4)ccc2-c2c1ccc1c2CCCC1 ; CC1(C)c2cc(N(c3cccc(-c4ccccc4)c3)c3ccccc3-c3ccccc3-c3ccccc3)ccc2-c2c1ccc1c2CCCC1 ; CC1(C)c2cc(N(c3ccccc3-c3ccccc3)c3ccccc3-c3ccccc3-c3ccccc3)ccc2-c2c1ccc1c2CCCC1]

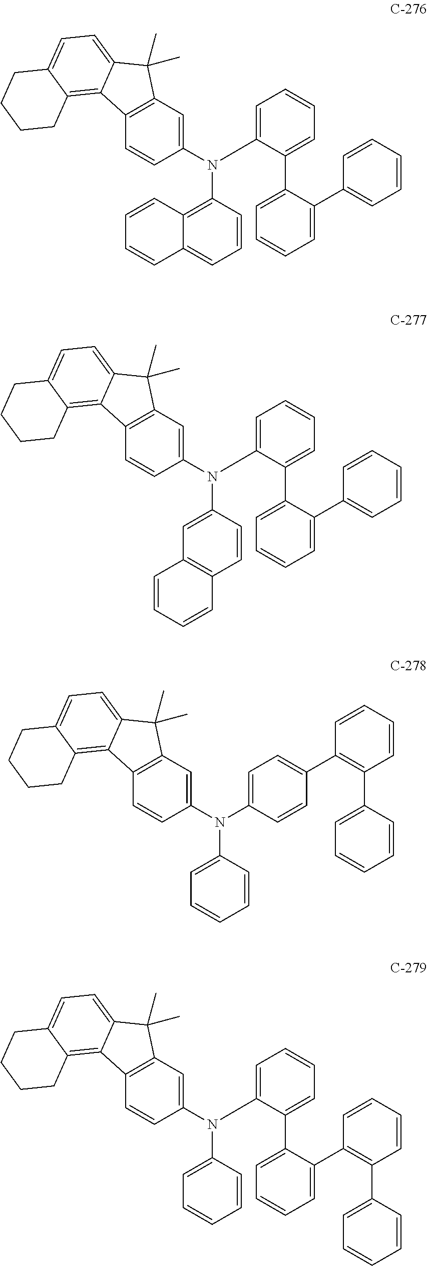 [SMILES: CC1(C)c2cc(N(c3ccccc3-c3ccccc3-c3ccccc3)c3cccc4ccccc34)ccc2-c2c1ccc1c2CCCC1 ; CC1(C)c2cc(N(c3ccc4ccccc4c3)c3ccccc3-c3ccccc3-c3ccccc3)ccc2-c2c1ccc1c2CCCC1 ; CC1(C)c2cc(N(c3ccccc3)c3ccc(-c4ccccc4-c4ccccc4)cc3)ccc2-c2c1ccc1c2CCCC1 ; CC1(C)c2cc(N(c3ccccc3)c3ccccc3-c3ccccc3-c3ccccc3-c3ccccc3)ccc2-c2c1ccc1c2CCCC1]

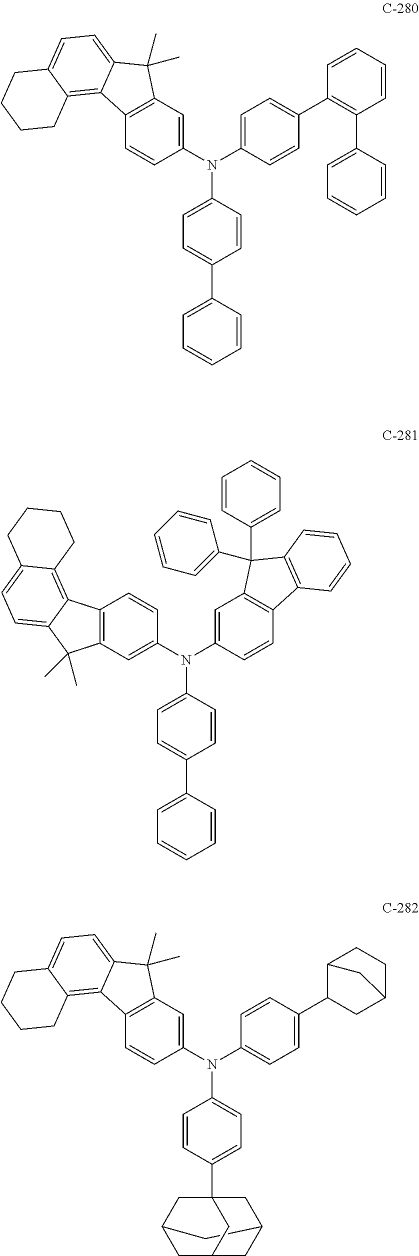 [SMILES: CC1(C)c2cc(N(c3ccc(-c4ccccc4)cc3)c3ccc(-c4ccccc4-c4ccccc4)cc3)ccc2-c2c1ccc1c2CCCC1 ; CC1(C)c2cc(N(c3ccc(-c4ccccc4)cc3)c3ccc4c(c3)C(c3ccccc3)(c3ccccc3)c3ccccc3-4)ccc2-c2c1ccc1c2CCCC1 ; CC1(C)c2cc(N(c3ccc(C4CC5CCC4C5)cc3)c3ccc(C45CC6CC(CC(C6)C4)C5)cc3)ccc2-c2c1ccc1c2CCCC1]

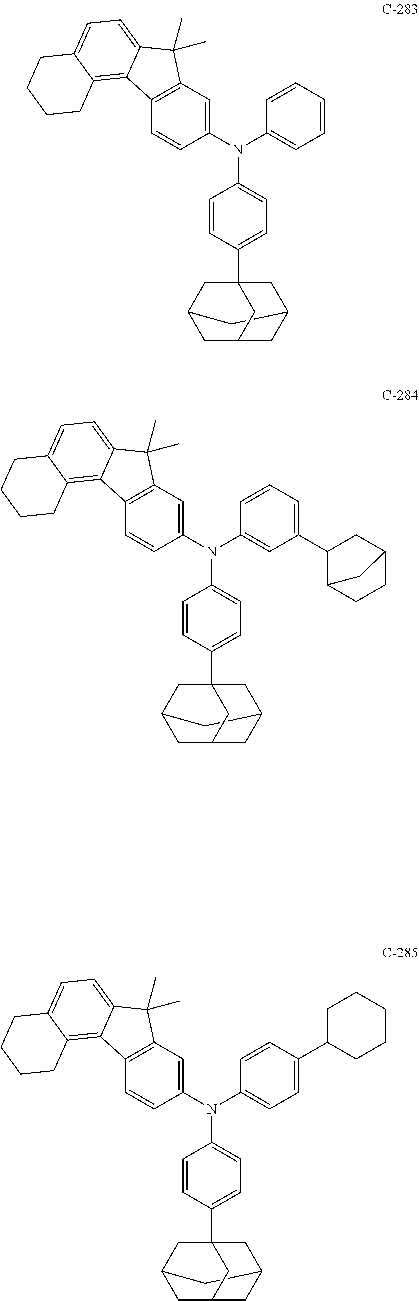 [SMILES: CC1(C)c2cc(N(c3ccccc3)c3ccc(C45CC6CC(CC(C6)C4)C5)cc3)ccc2-c2c1ccc1c2CCCC1 ; CC1(C)c2cc(N(c3ccc(C45CC6CC(CC(C6)C4)C5)cc3)c3cccc(C4CC5CCC4C5)c3)ccc2-c2c1ccc1c2CCCC1 ; CC1(C)c2cc(N(c3ccc(C4CCCCC4)cc3)c3ccc(C45CC6CC(CC(C6)C4)C5)cc3)ccc2-c2c1ccc1c2CCCC1]

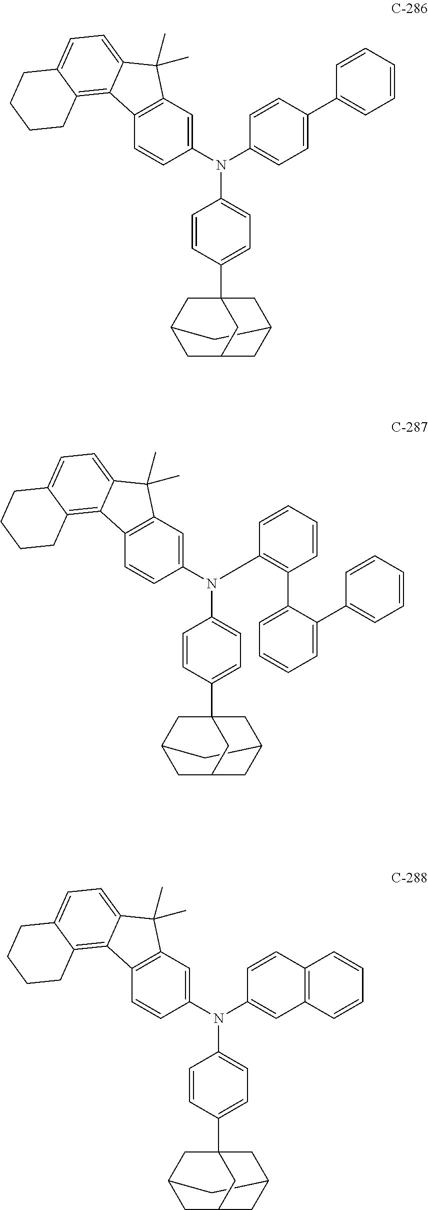 [SMILES: CC1(C)c2cc(N(c3ccc(-c4ccccc4)cc3)c3ccc(C45CC6CC(CC(C6)C4)C5)cc3)ccc2-c2c1ccc1c2CCCC1 ; CC1(C)c2cc(N(c3ccc(C45CC6CC(CC(C6)C4)C5)cc3)c3ccccc3-c3ccccc3-c3ccccc3)ccc2-c2c1ccc1c2CCCC1 ; CC1(C)c2cc(N(c3ccc(C45CC6CC(CC(C6)C4)C5)cc3)c3ccc4ccccc4c3)ccc2-c2c1ccc1c2CCCC1]

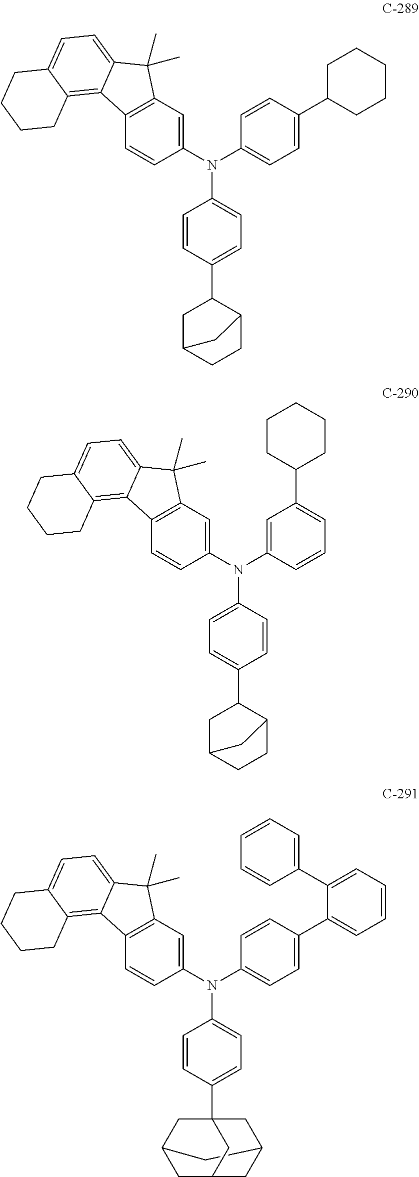 [SMILES: CC1(C)c2cc(N(c3ccc(C4CCCCC4)cc3)c3ccc(C4CC5CCC4C5)cc3)ccc2-c2c1ccc1c2CCCC1 ; CC1(C)c2cc(N(c3ccc(C4CC5CCC4C5)cc3)c3cccc(C4CCCCC4)c3)ccc2-c2c1ccc1c2CCCC1 ; CC1(C)c2cc(N(c3ccc(-c4ccccc4-c4ccccc4)cc3)c3ccc(C45CC6CC(CC(C6)C4)C5)cc3)ccc2-c2c1ccc1c2CCCC1]

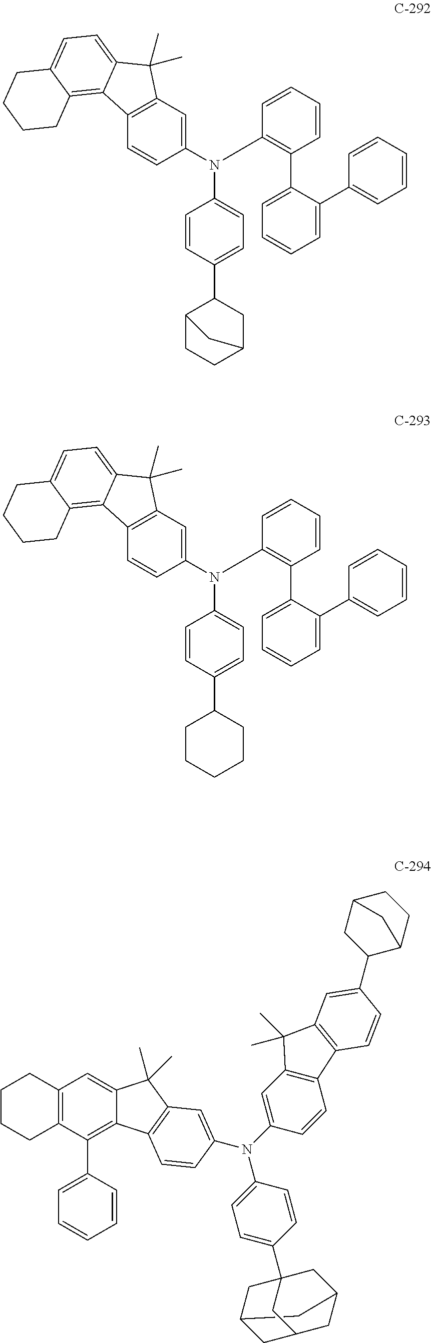 [SMILES: CC1(C)c2cc(N(c3ccc(C4CC5CCC4C5)cc3)c3ccccc3-c3ccccc3-c3ccccc3)ccc2-c2c1ccc1c2CCCC1 ; CC1(C)c2cc(N(c3ccc(C4CCCCC4)cc3)c3ccccc3-c3ccccc3-c3ccccc3)ccc2-c2c1ccc1c2CCCC1 ; CC1(C)c2cc(C3CC4CCC3C4)ccc2-c2ccc(N(c3ccc(C45CC6CC(CC(C6)C4)C5)cc3)c3ccc4c(c3)C(C)(C)c3cc5c(c(-c6ccccc6)c3-4)CCCC5)cc21]

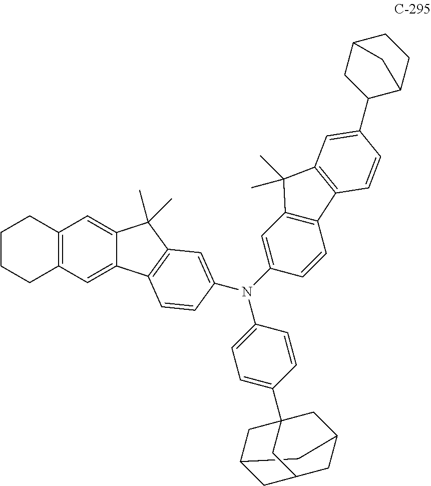 [SMILES: CC1(C)c2cc(C3CC4CCC3C4)ccc2-c2ccc(N(c3ccc(C45CC6CC(CC(C6)C4)C5)cc3)c3ccc4c(c3)C(C)(C)c3cc5c(cc3-4)CCCC5)cc21]

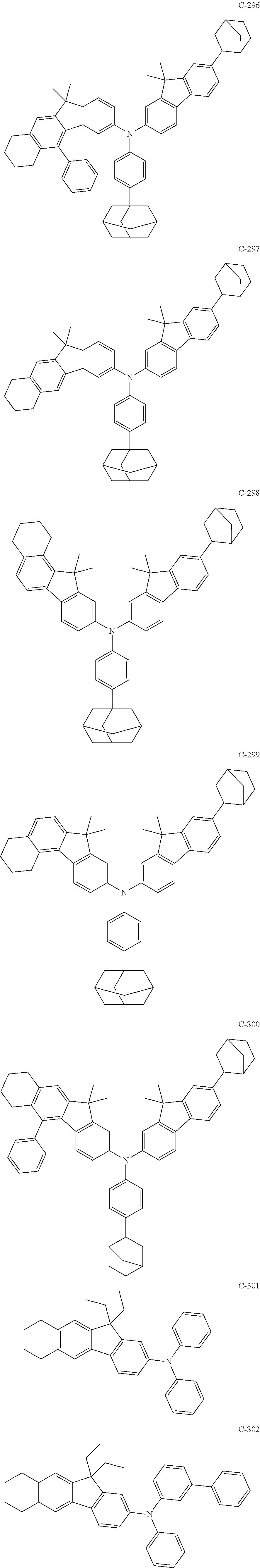

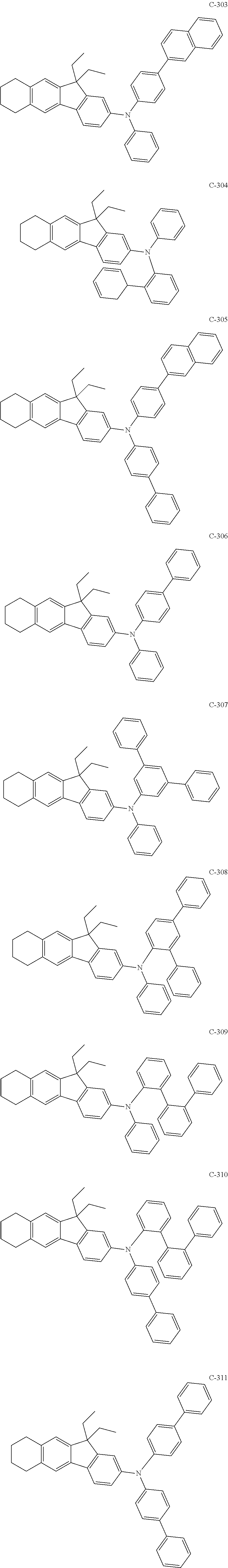

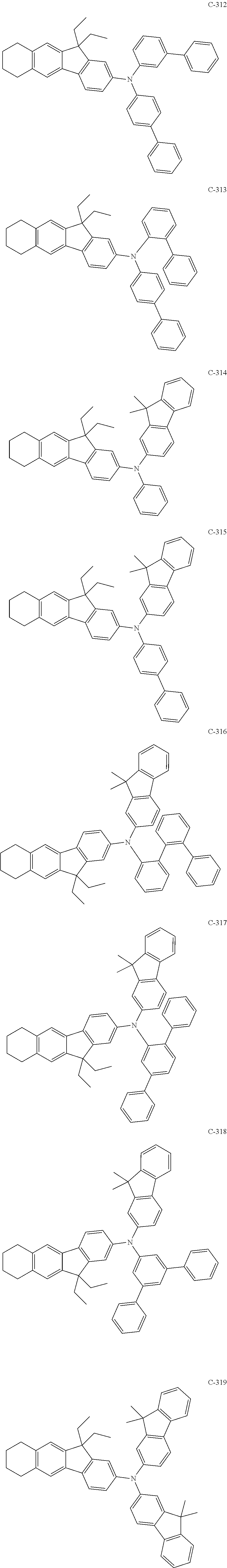

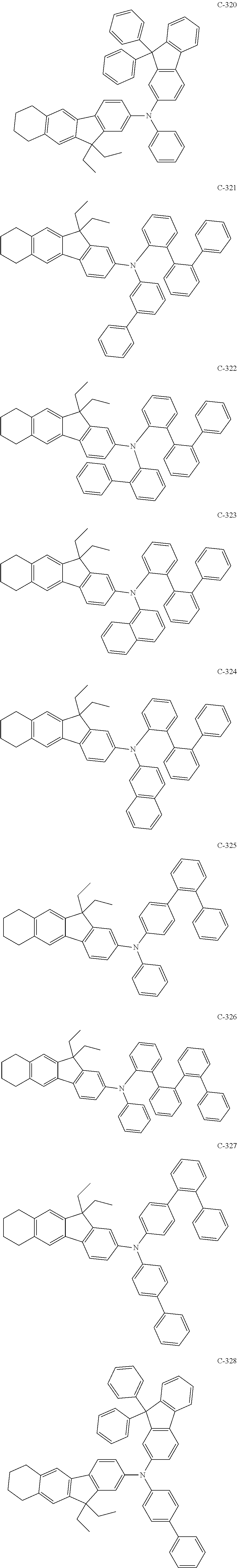

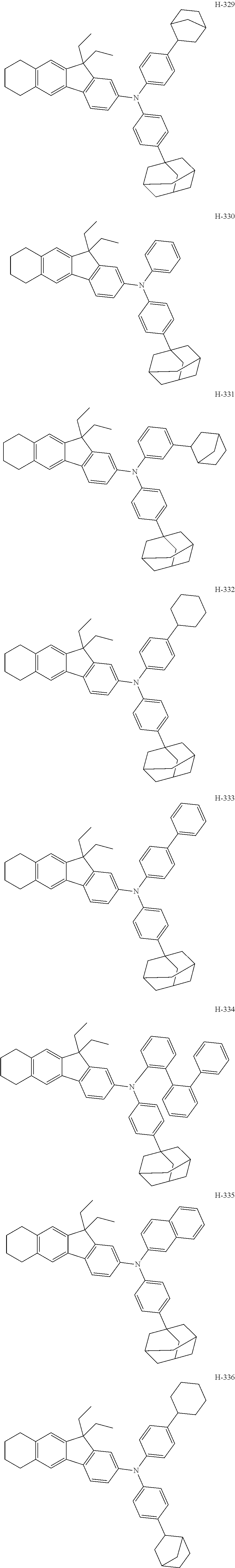

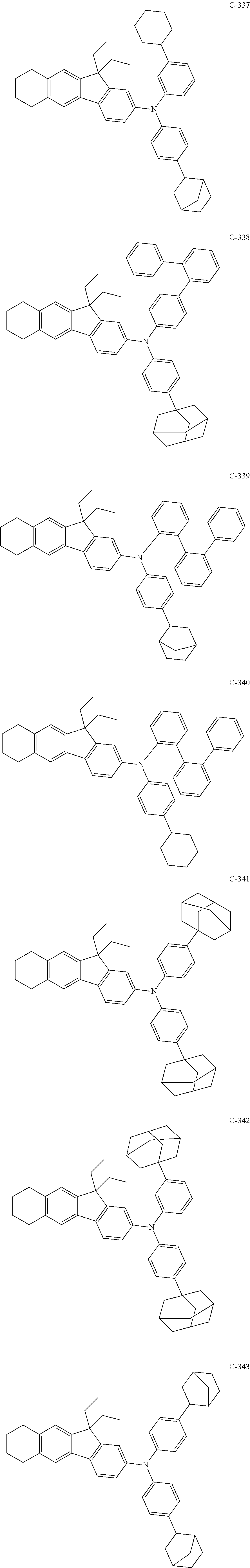

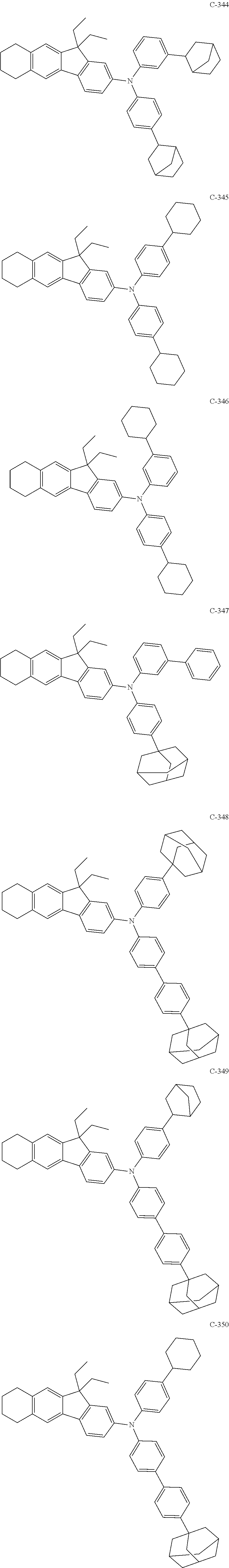

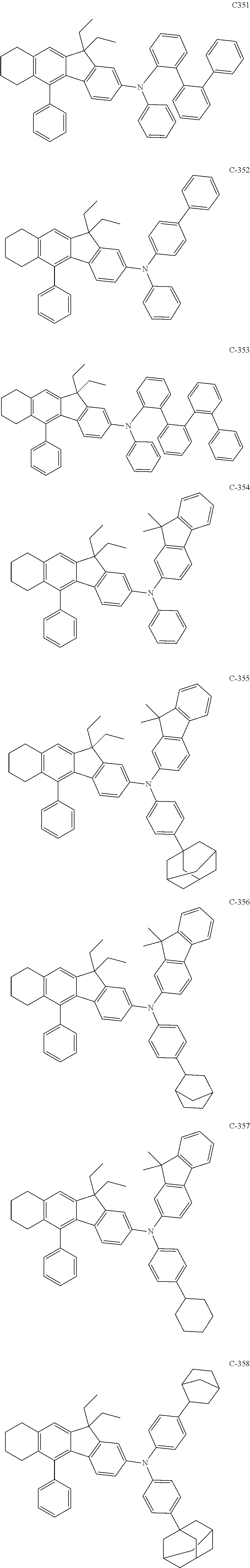

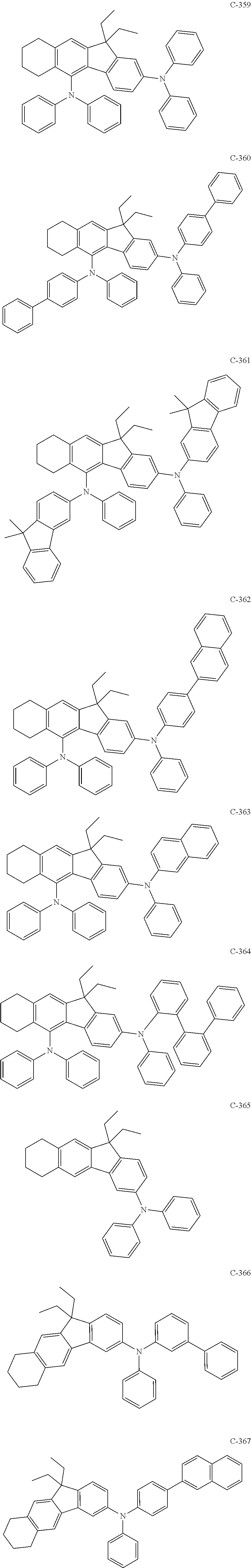

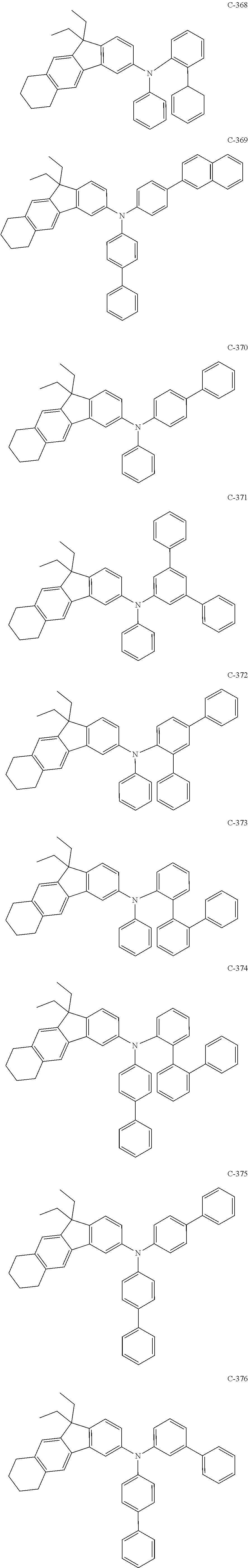

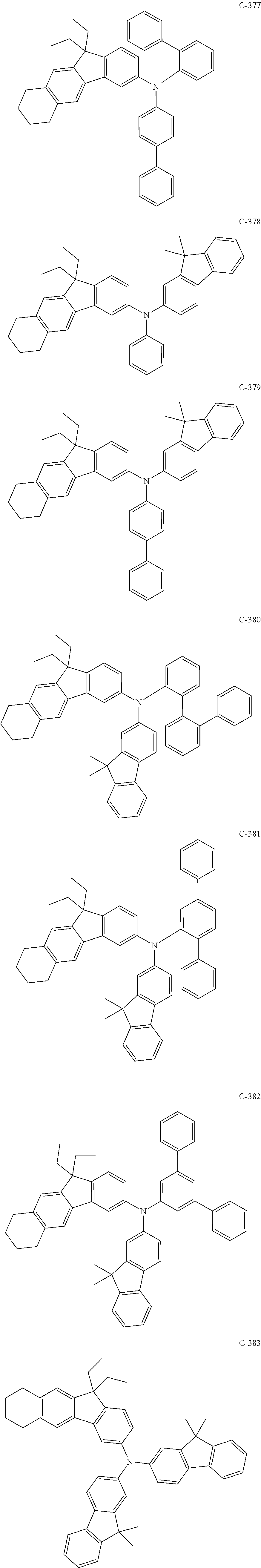

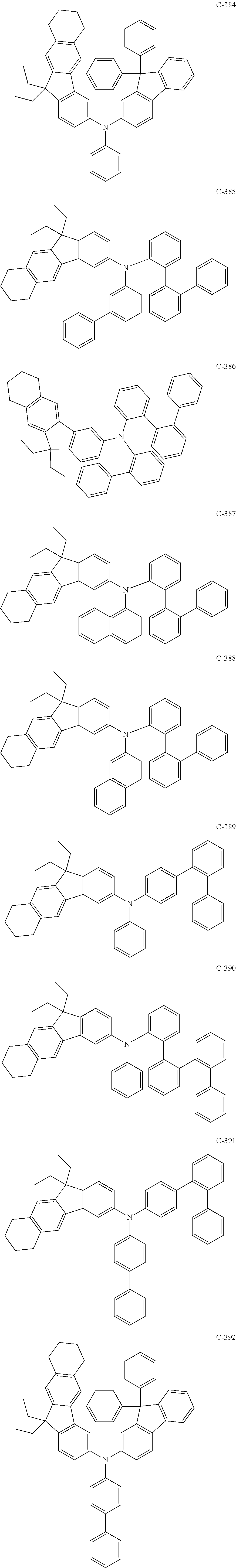

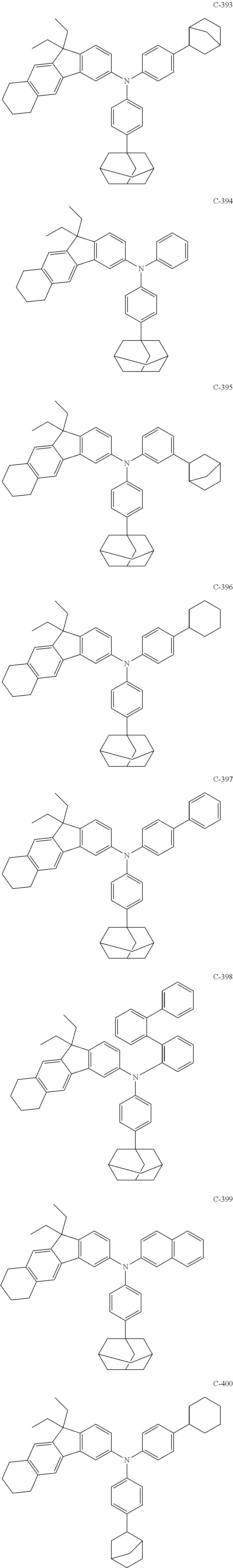

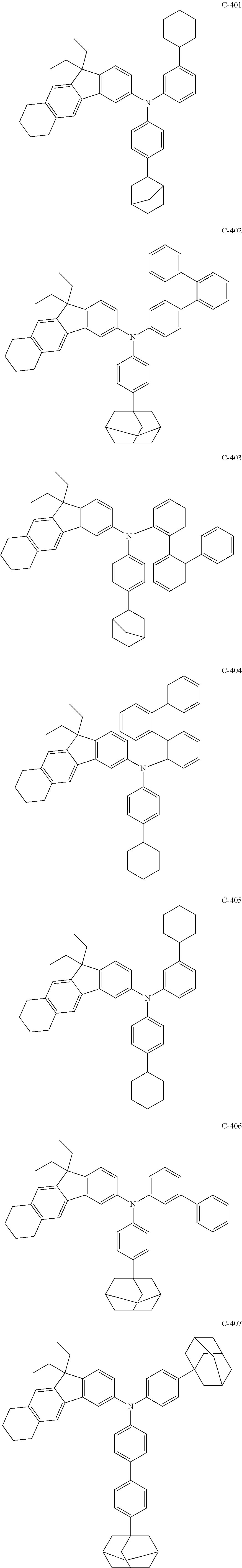

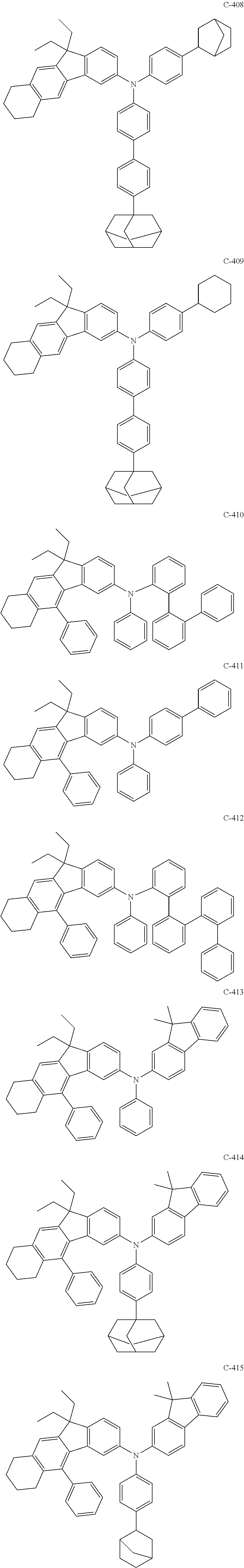

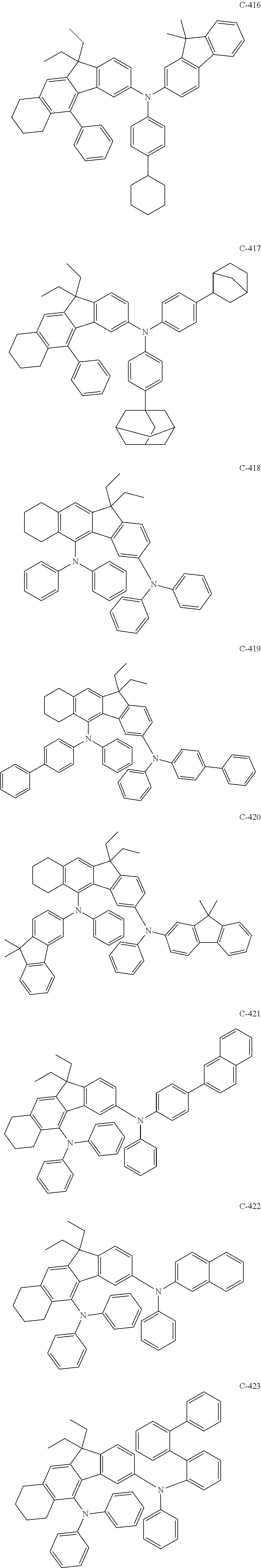

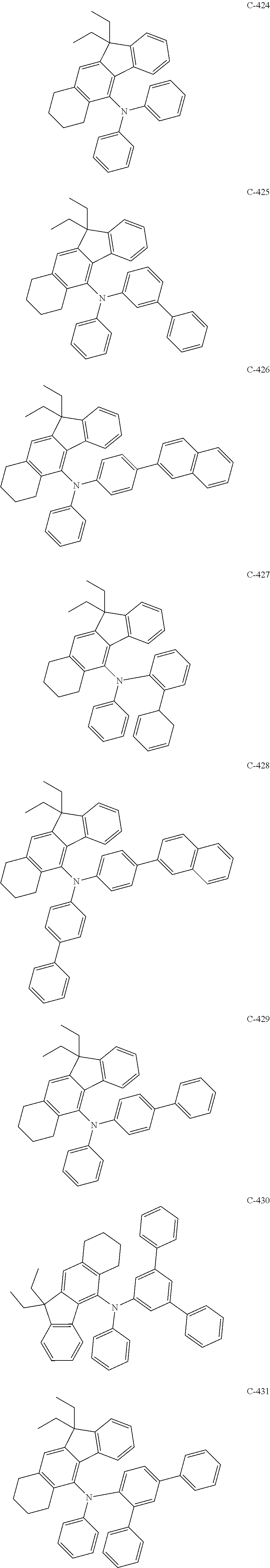

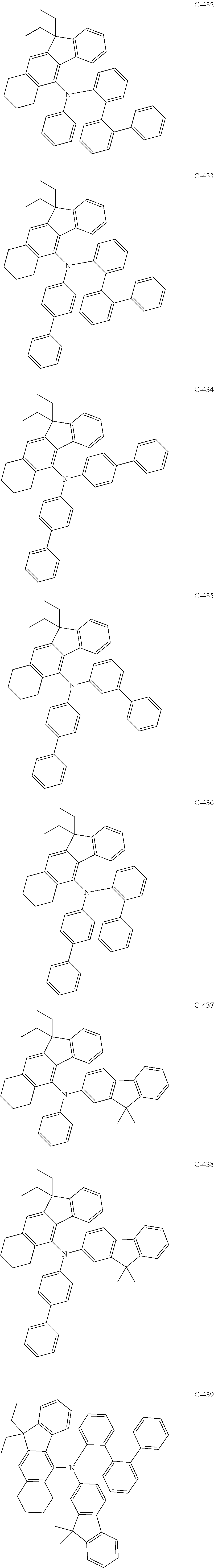

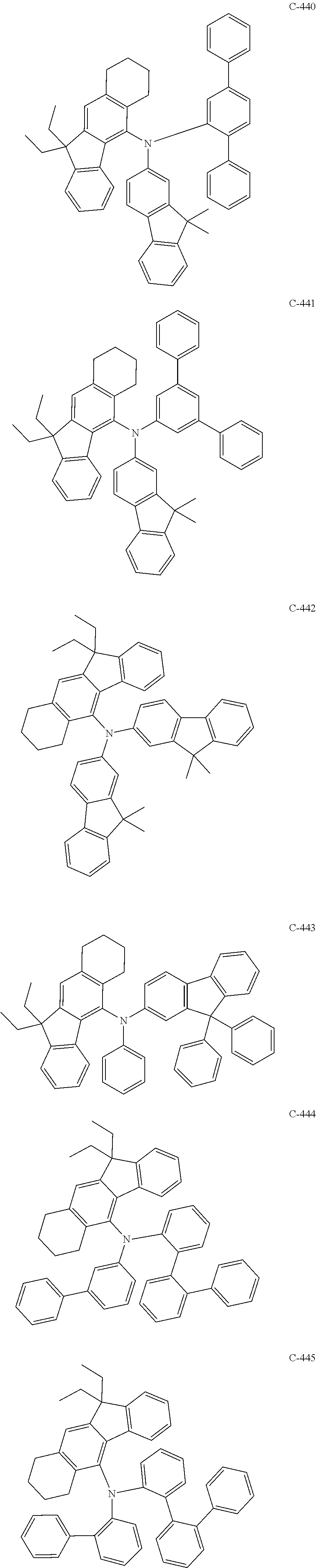

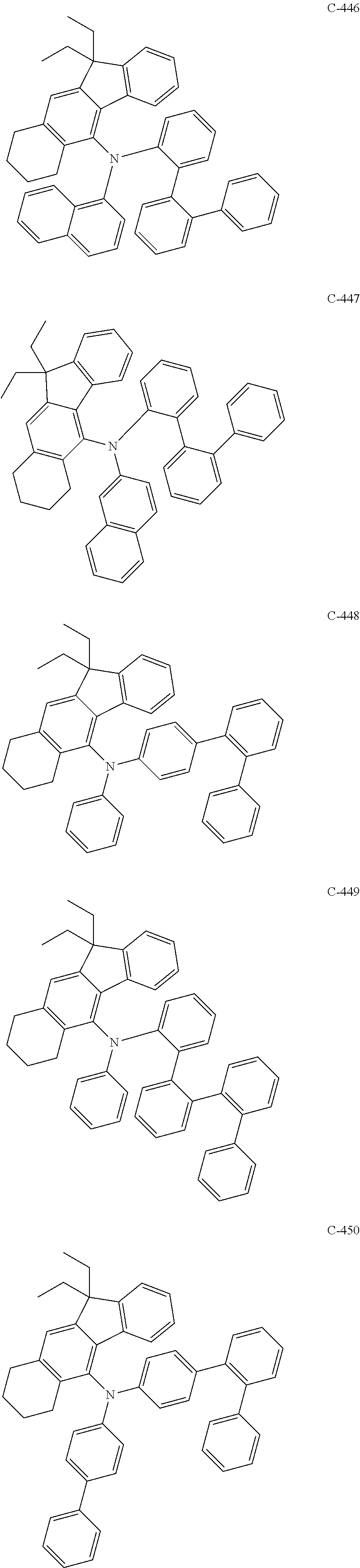 [SMILES: CCC1(CC)c2ccccc2-c2c1cc1c(c2N(c2ccccc2-c2ccccc2-c2ccccc2)c2cccc3ccccc23)CCCC1 ; CCC1(CC)c2ccccc2-c2c1cc1c(c2N(c2ccc3ccccc3c2)c2ccccc2-c2ccccc2-c2ccccc2)CCCC1 ; CCC1(CC)c2ccccc2-c2c1cc1c(c2N(c2ccccc2)c2ccc(-c3ccccc3-c3ccccc3)cc2)CCCC1 ; CCC1(CC)c2ccccc2-c2c1cc1c(c2N(c2ccccc2)c2ccccc2-c2ccccc2-c2ccccc2-c2ccccc2)CCCC1 ; CCC1(CC)c2ccccc2-c2c1cc1c(c2N(c2ccc(-c3ccccc3)cc2)c2ccc(-c3ccccc3-c3ccccc3)cc2)CCCC1]

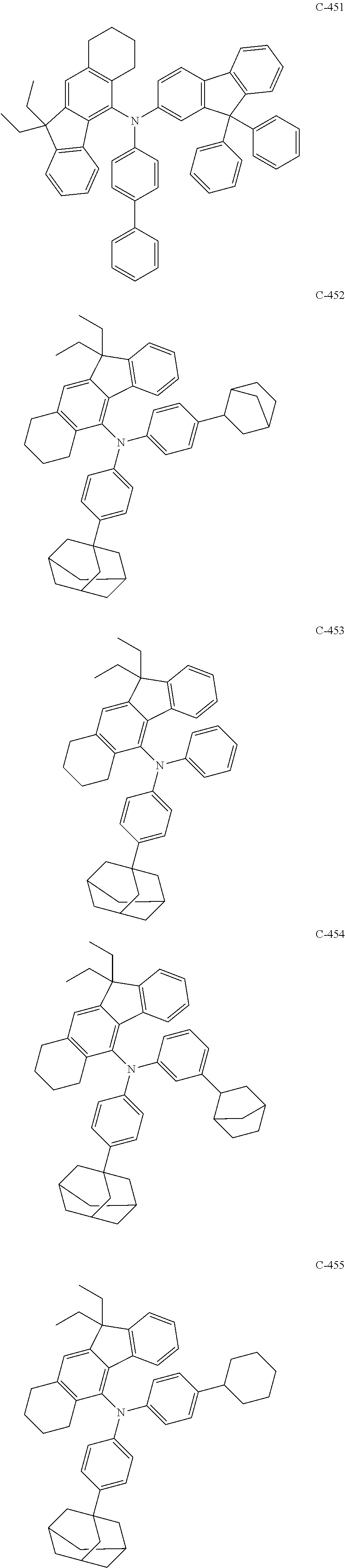

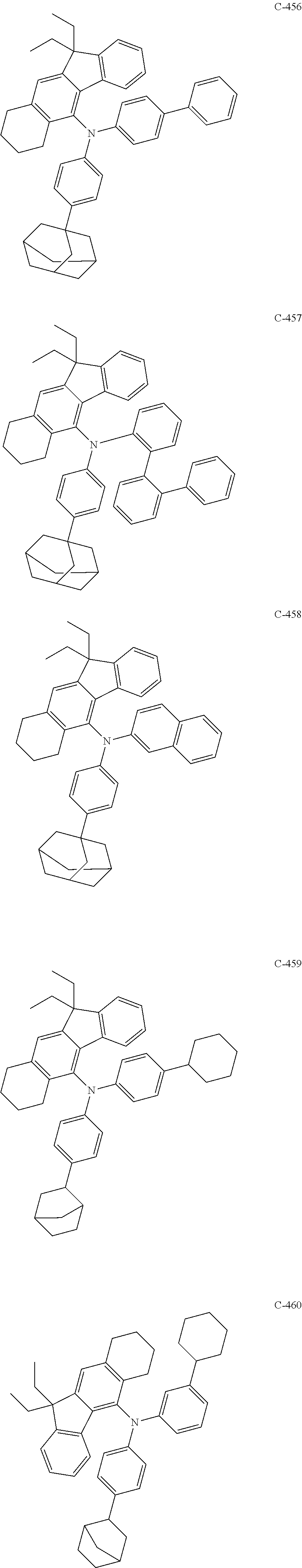

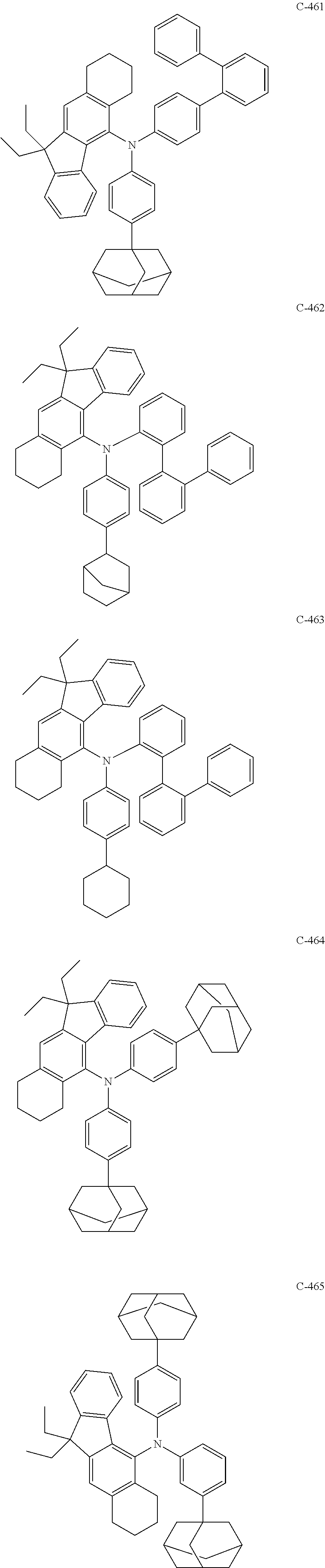 [SMILES: CCC1(CC)c2ccccc2-c2c1cc1c(c2N(c2ccc(-c3ccccc3-c3ccccc3)cc2)c2ccc(C34CC5CC(CC(C5)C3)C4)cc2)CCCC1 ; CCC1(CC)c2ccccc2-c2c1cc1c(c2N(c2ccc(C3CC4CCC3C4)cc2)c2ccccc2-c2ccccc2-c2ccccc2)CCCC1 ; CCC1(CC)c2ccccc2-c2c1cc1c(c2N(c2ccc(C3CCCCC3)cc2)c2ccccc2-c2ccccc2-c2ccccc2)CCCC1 ; CCC1(CC)c2ccccc2-c2c1cc1c(c2N(c2ccc(C34CC5CC(CC(C5)C3)C4)cc2)c2ccc(C34CC5CC(CC(C5)C3)C4)cc2)CCCC1 ; CCC1(CC)c2ccccc2-c2c1cc1c(c2N(c2ccc(C34CC5CC(CC(C5)C3)C4)cc2)c2cccc(C34CC5CC(CC(C5)C3)C4)c2)CCCC1]

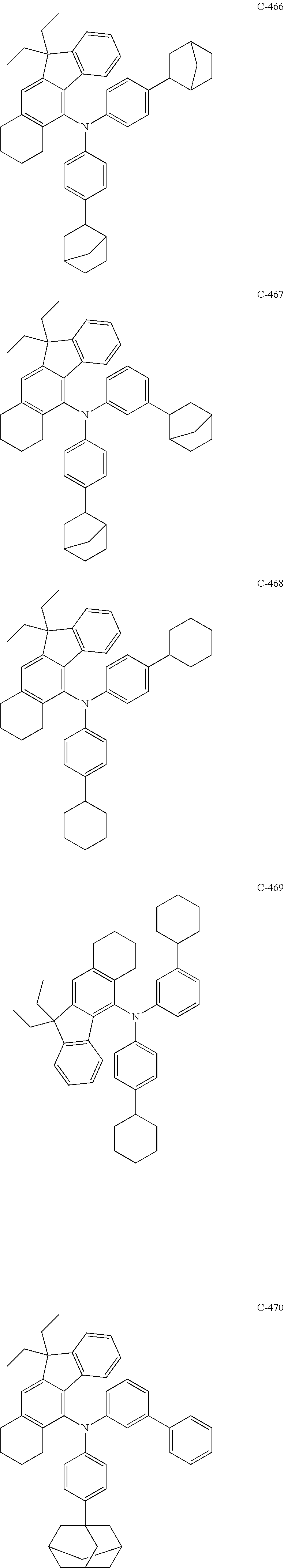

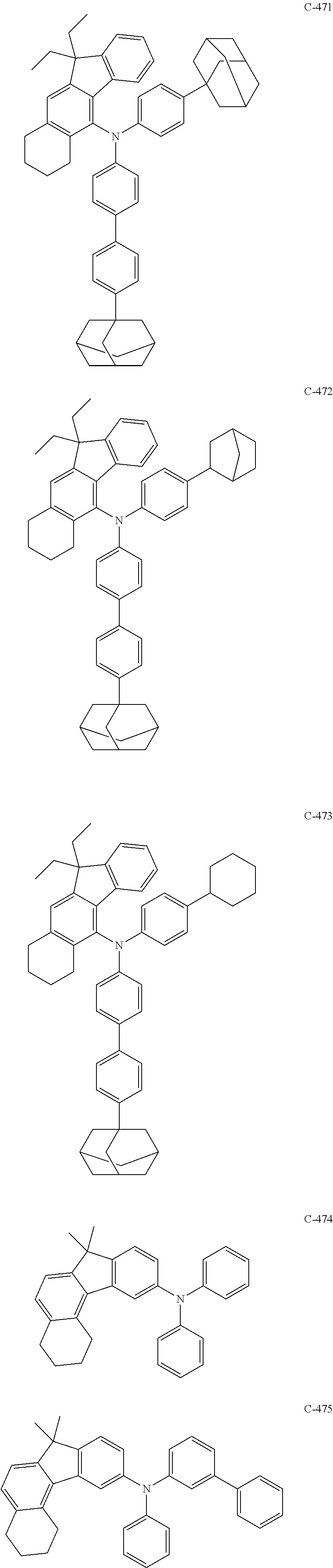

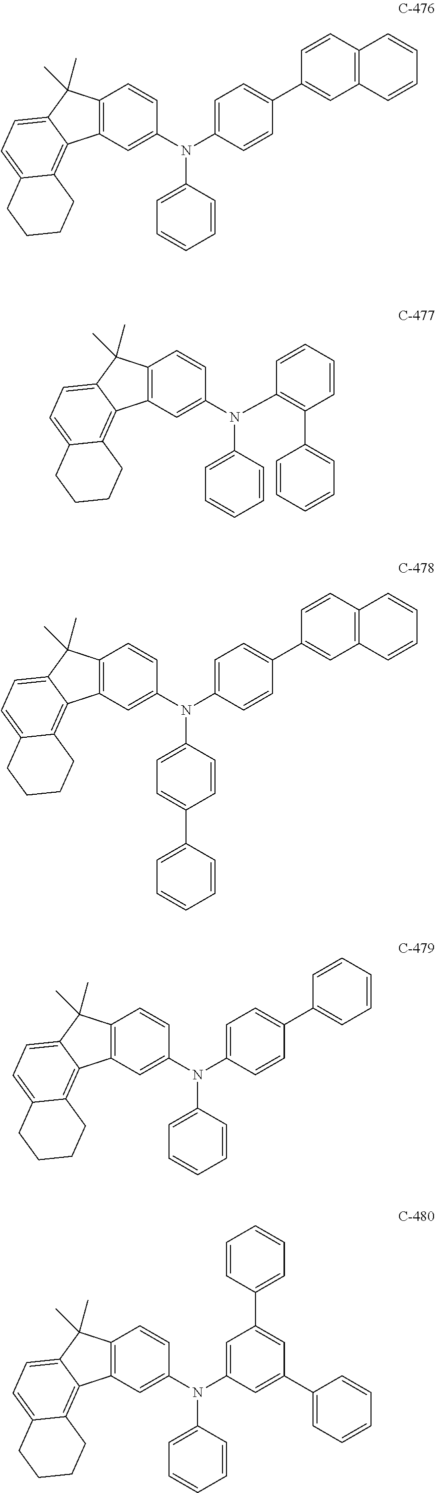 [SMILES: CC1(C)c2ccc(N(c3ccccc3)c3ccc(-c4ccc5ccccc5c4)cc3)cc2-c2c1ccc1c2CCCC1 ; CC1(C)c2ccc(N(c3ccccc3)c3ccccc3-c3ccccc3)cc2-c2c1ccc1c2CCCC1 ; CC1(C)c2ccc(N(c3ccc(-c4ccccc4)cc3)c3ccc(-c4ccc5ccccc5c4)cc3)cc2-c2c1ccc1c2CCCC1 ; CC1(C)c2ccc(N(c3ccccc3)c3ccc(-c4ccccc4)cc3)cc2-c2c1ccc1c2CCCC1 ; CC1(C)c2ccc(N(c3ccccc3)c3cc(-c4ccccc4)cc(-c4ccccc4)c3)cc2-c2c1ccc1c2CCCC1]

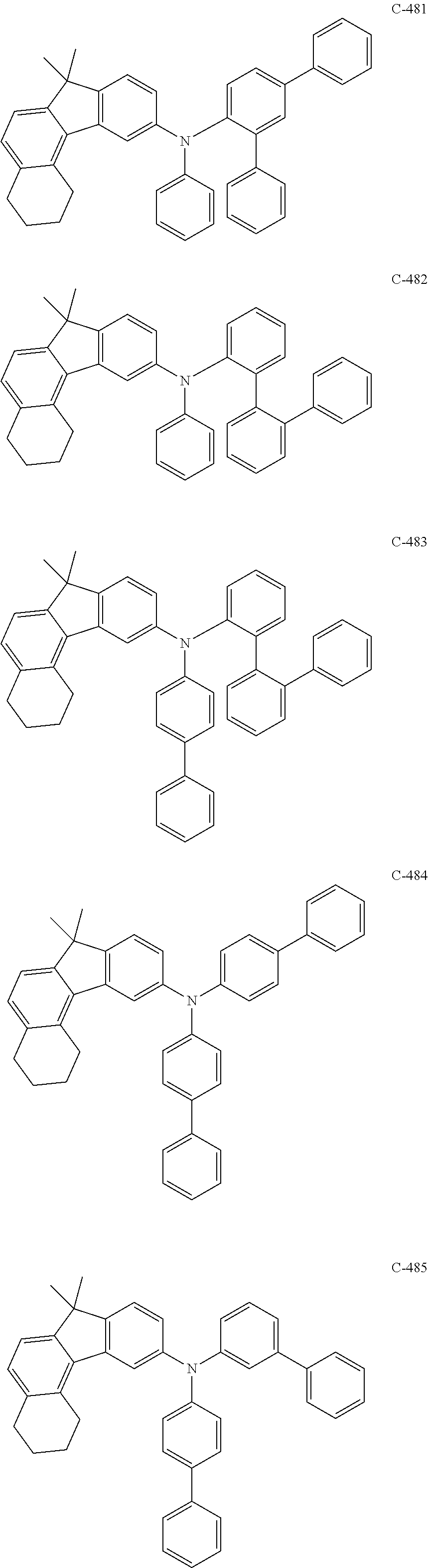 [SMILES: CC1(C)c2ccc(N(c3ccccc3)c3ccc(-c4ccccc4)cc3-c3ccccc3)cc2-c2c1ccc1c2CCCC1 ; CC1(C)c2ccc(N(c3ccccc3)c3ccccc3-c3ccccc3-c3ccccc3)cc2-c2c1ccc1c2CCCC1 ; CC1(C)c2ccc(N(c3ccc(-c4ccccc4)cc3)c3ccccc3-c3ccccc3-c3ccccc3)cc2-c2c1ccc1c2CCCC1 ; CC1(C)c2ccc(N(c3ccc(-c4ccccc4)cc3)c3ccc(-c4ccccc4)cc3)cc2-c2c1ccc1c2CCCC1 ; CC1(C)c2ccc(N(c3ccc(-c4ccccc4)cc3)c3cccc(-c4ccccc4)c3)cc2-c2c1ccc1c2CCCC1]

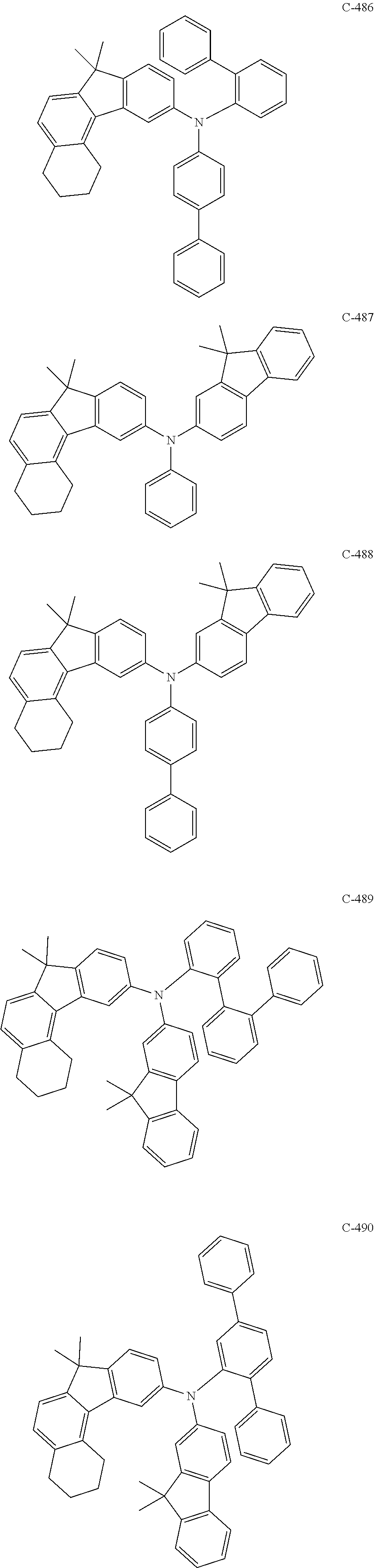

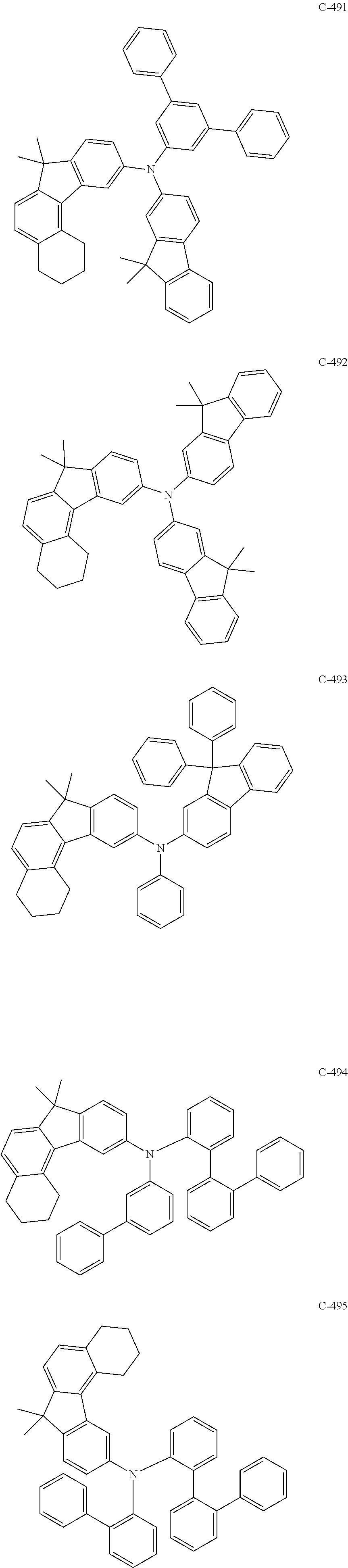 [SMILES: CC1(C)c2ccccc2-c2ccc(N(c3cc(-c4ccccc4)cc(-c4ccccc4)c3)c3ccc4c(c3)-c3c(ccc5c3CCCC5)C4(C)C)cc21 ; CC1(C)c2ccccc2-c2ccc(N(c3ccc4c(c3)-c3c(ccc5c3CCCC5)C4(C)C)c3ccc4c(c3)C(C)(C)c3ccccc3-4)cc21 ; CC1(C)c2ccc(N(c3ccccc3)c3ccc4c(c3)C(c3ccccc3)(c3ccccc3)c3ccccc3-4)cc2-c2c1ccc1c2CCCC1 ; CC1(C)c2ccc(N(c3cccc(-c4ccccc4)c3)c3ccccc3-c3ccccc3-c3ccccc3)cc2-c2c1ccc1c2CCCC1 ; CC1(C)c2ccc(N(c3ccccc3-c3ccccc3)c3ccccc3-c3ccccc3-c3ccccc3)cc2-c2c1ccc1c2CCCC1]

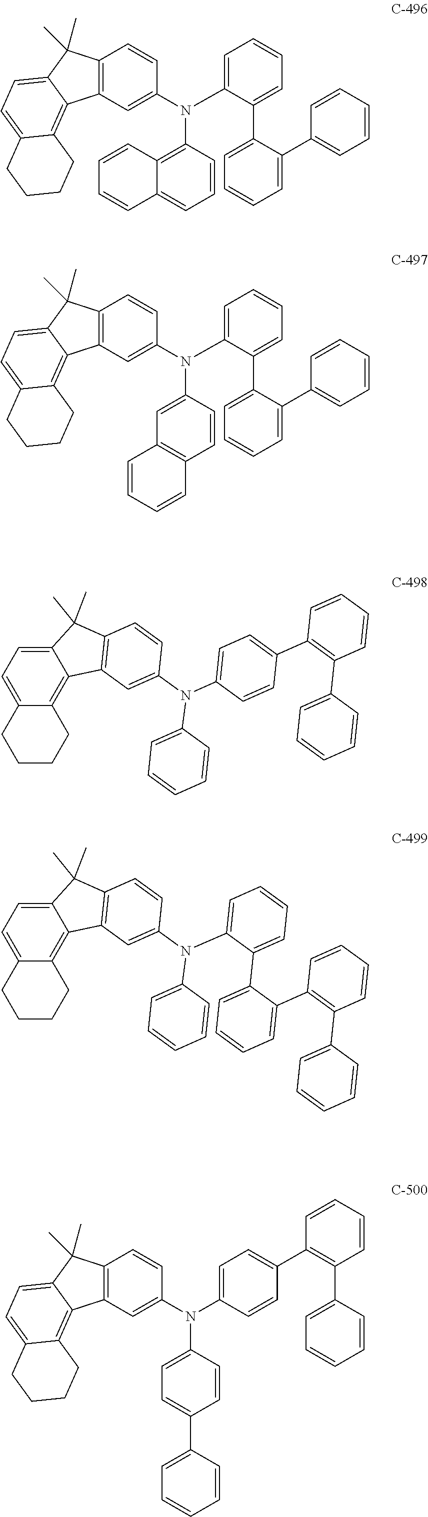 [SMILES: CC1(C)c2ccc(N(c3ccccc3-c3ccccc3-c3ccccc3)c3cccc4ccccc34)cc2-c2c1ccc1c2CCCC1 ; CC1(C)c2ccc(N(c3ccc4ccccc4c3)c3ccccc3-c3ccccc3-c3ccccc3)cc2-c2c1ccc1c2CCCC1 ; CC1(C)c2ccc(N(c3ccccc3)c3ccc(-c4ccccc4-c4ccccc4)cc3)cc2-c2c1ccc1c2CCCC1 ; CC1(C)c2ccc(N(c3ccccc3)c3ccccc3-c3ccccc3-c3ccccc3-c3ccccc3)cc2-c2c1ccc1c2CCCC1 ; CC1(C)c2ccc(N(c3ccc(-c4ccccc4)cc3)c3ccc(-c4ccccc4-c4ccccc4)cc3)cc2-c2c1ccc1c2CCCC1]

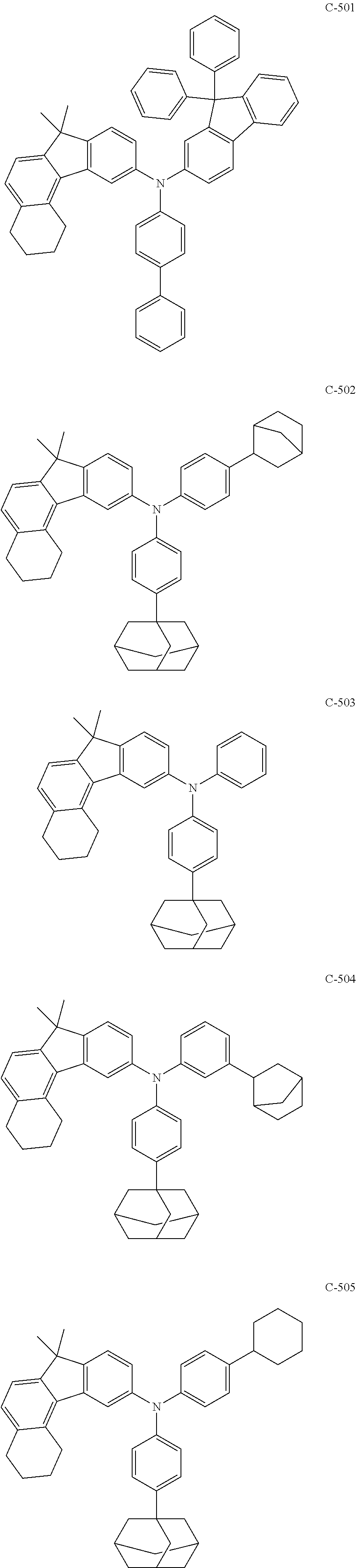 [SMILES: CC1(C)c2ccc(N(c3ccc(-c4ccccc4)cc3)c3ccc4c(c3)C(c3ccccc3)(c3ccccc3)c3ccccc3-4)cc2-c2c1ccc1c2CCCC1 ; CC1(C)c2ccc(N(c3ccc(C4CC5CCC4C5)cc3)c3ccc(C45CC6CC(CC(C6)C4)C5)cc3)cc2-c2c1ccc1c2CCCC1 ; CC1(C)c2ccc(N(c3ccccc3)c3ccc(C45CC6CC(CC(C6)C4)C5)cc3)cc2-c2c1ccc1c2CCCC1 ; CC1(C)c2ccc(N(c3ccc(C45CC6CC(CC(C6)C4)C5)cc3)c3cccc(C4CC5CCC4C5)c3)cc2-c2c1ccc1c2CCCC1 ; CC1(C)c2ccc(N(c3ccc(C4CCCCC4)cc3)c3ccc(C45CC6CC(CC(C6)C4)C5)cc3)cc2-c2c1ccc1c2CCCC1]

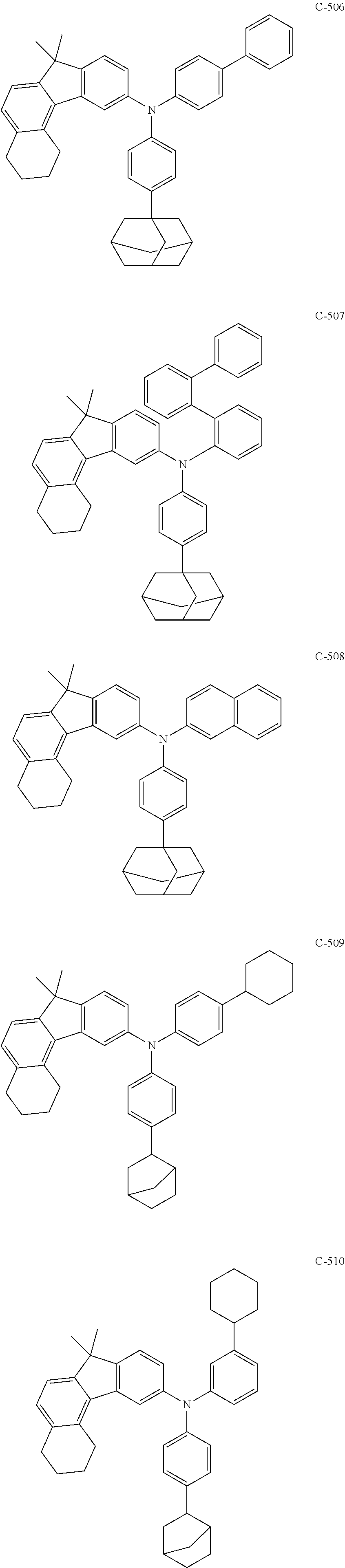 [SMILES: CC1(C)c2ccc(N(c3ccc(-c4ccccc4)cc3)c3ccc(C45CC6CC(CC(C6)C4)C5)cc3)cc2-c2c1ccc1c2CCCC1 ; CC1(C)c2ccc(N(c3ccc(C45CC6CC(CC(C6)C4)C5)cc3)c3ccccc3-c3ccccc3-c3ccccc3)cc2-c2c1ccc1c2CCCC1 ; CC1(C)c2ccc(N(c3ccc(C45CC6CC(CC(C6)C4)C5)cc3)c3ccc4ccccc4c3)cc2-c2c1ccc1c2CCCC1 ; CC1(C)c2ccc(N(c3ccc(C4CCCCC4)cc3)c3ccc(C4CC5CCC4C5)cc3)cc2-c2c1ccc1c2CCCC1 ; CC1(C)c2ccc(N(c3ccc(C4CC5CCC4C5)cc3)c3cccc(C4CCCCC4)c3)cc2-c2c1ccc1c2CCCC1]

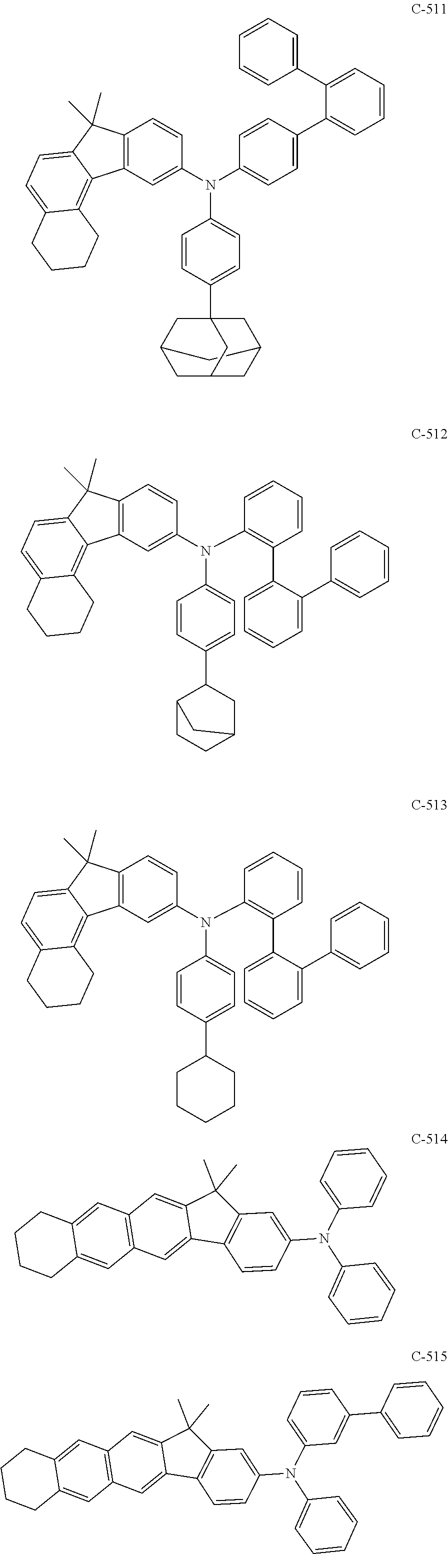

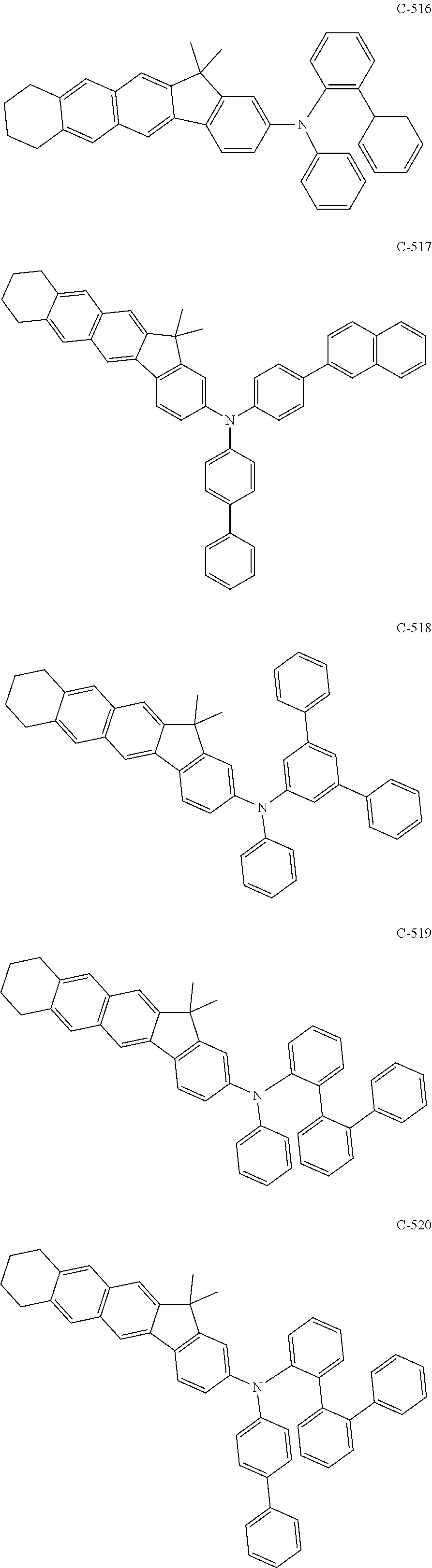 [SMILES: CC1(C)c2cc(N(c3ccccc3)c3ccccc3C3C=CC=CC3)ccc2-c2cc3cc4c(cc3cc21)CCCC4 ; CC1(C)c2cc(N(c3ccc(-c4ccccc4)cc3)c3ccc(-c4ccc5ccccc5c4)cc3)ccc2-c2cc3cc4c(cc3cc21)CCCC4 ; CC1(C)c2cc(N(c3ccccc3)c3cc(-c4ccccc4)cc(-c4ccccc4)c3)ccc2-c2cc3cc4c(cc3cc21)CCCC4 ; CC1(C)c2cc(N(c3ccccc3)c3ccccc3-c3ccccc3-c3ccccc3)ccc2-c2cc3cc4c(cc3cc21)CCCC4 ; CC1(C)c2cc(N(c3ccc(-c4ccccc4)cc3)c3ccccc3-c3ccccc3-c3ccccc3)ccc2-c2cc3cc4c(cc3cc21)CCCC4]

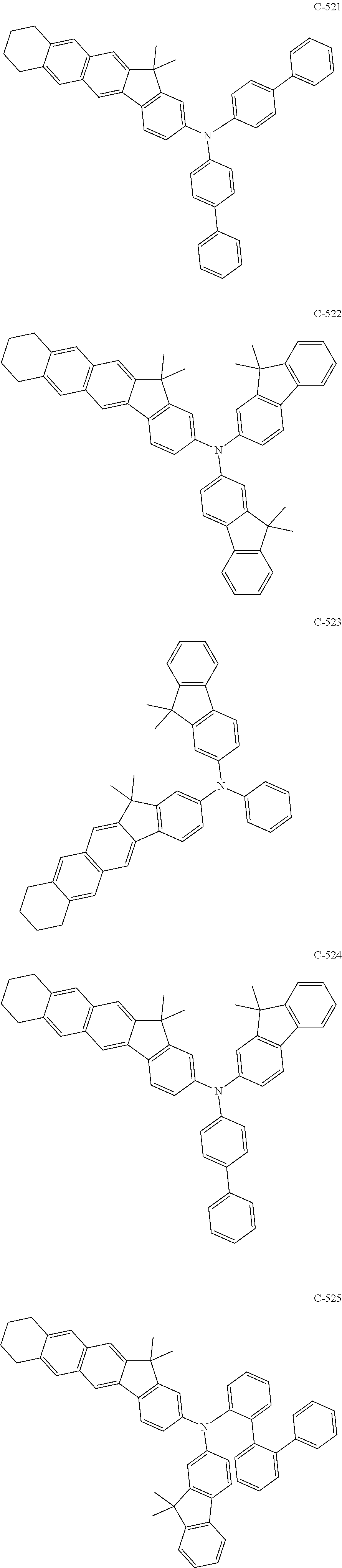 [SMILES: CC1(C)c2cc(N(c3ccc(-c4ccccc4)cc3)c3ccc(-c4ccccc4)cc3)ccc2-c2cc3cc4c(cc3cc21)CCCC4 ; CC1(C)c2ccccc2-c2ccc(N(c3ccc4c(c3)C(C)(C)c3ccccc3-4)c3ccc4c(c3)C(C)(C)c3cc5cc6c(cc5cc3-4)CCCC6)cc21 ; CC1(C)c2ccccc2-c2ccc(N(c3ccccc3)c3ccc4c(c3)C(C)(C)c3cc5cc6c(cc5cc3-4)CCCC6)cc21 ; CC1(C)c2ccccc2-c2ccc(N(c3ccc(-c4ccccc4)cc3)c3ccc4c(c3)C(C)(C)c3cc5cc6c(cc5cc3-4)CCCC6)cc21 ; CC1(C)c2ccccc2-c2ccc(N(c3ccc4c(c3)C(C)(C)c3cc5cc6c(cc5cc3-4)CCCC6)c3ccccc3-c3ccccc3-c3ccccc3)cc21]

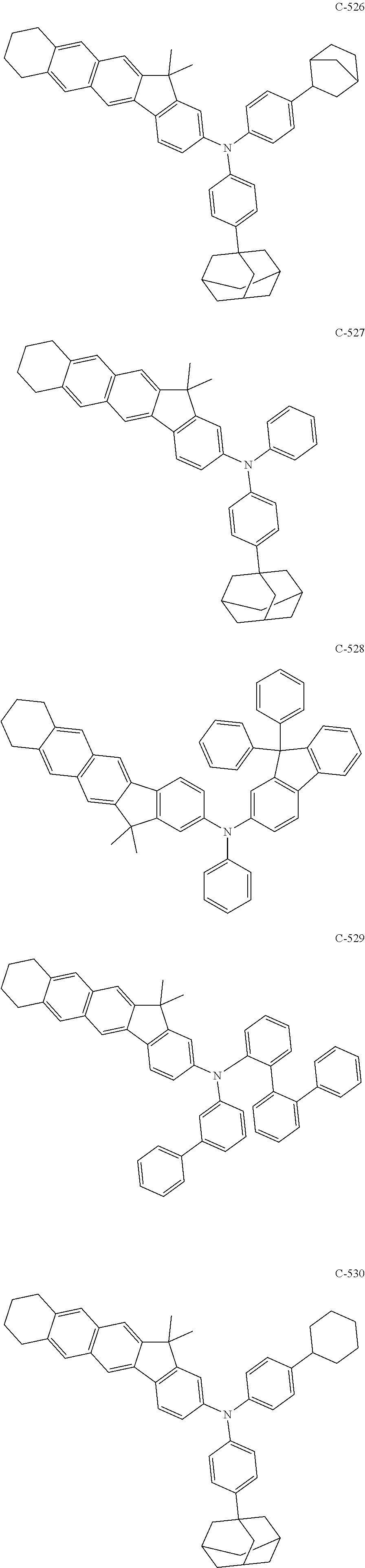 [SMILES: CC1(C)c2cc(N(c3ccc(C4CC5CCC4C5)cc3)c3ccc(C45CC6CC(CC(C6)C4)C5)cc3)ccc2-c2cc3cc4c(cc3cc21)CCCC4 ; CC1(C)c2cc(N(c3ccccc3)c3ccc(C45CC6CC(CC(C6)C4)C5)cc3)ccc2-c2cc3cc4c(cc3cc21)CCCC4 ; CC1(C)c2cc(N(c3ccccc3)c3ccc4c(c3)C(c3ccccc3)(c3ccccc3)c3ccccc3-4)ccc2-c2cc3cc4c(cc3cc21)CCCC4 ; CC1(C)c2cc(N(c3cccc(-c4ccccc4)c3)c3ccccc3-c3ccccc3-c3ccccc3)ccc2-c2cc3cc4c(cc3cc21)CCCC4 ; CC1(C)c2cc(N(c3ccc(C4CCCCC4)cc3)c3ccc(C45CC6CC(CC(C6)C4)C5)cc3)ccc2-c2cc3cc4c(cc3cc21)CCCC4]

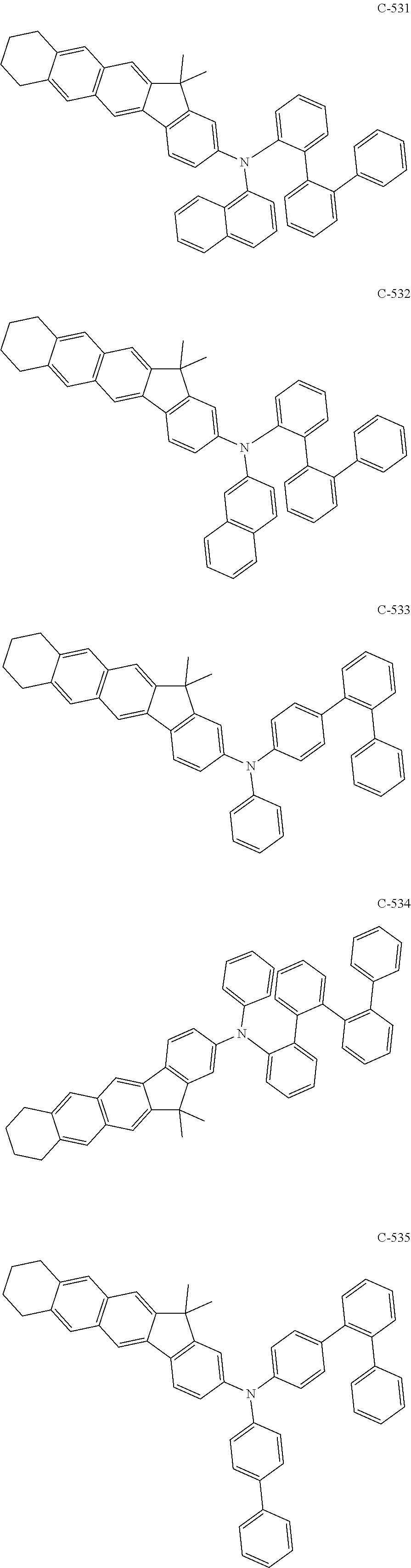 [SMILES: CC1(C)c2cc(N(c3ccccc3-c3ccccc3-c3ccccc3)c3cccc4ccccc34)ccc2-c2cc3cc4c(cc3cc21)CCCC4 ; CC1(C)c2cc(N(c3ccc4ccccc4c3)c3ccccc3-c3ccccc3-c3ccccc3)ccc2-c2cc3cc4c(cc3cc21)CCCC4 ; CC1(C)c2cc(N(c3ccccc3)c3ccc(-c4ccccc4-c4ccccc4)cc3)ccc2-c2cc3cc4c(cc3cc21)CCCC4 ; CC1(C)c2cc(N(c3ccccc3)c3ccccc3-c3ccccc3-c3ccccc3-c3ccccc3)ccc2-c2cc3cc4c(cc3cc21)CCCC4 ; CC1(C)c2cc(N(c3ccc(-c4ccccc4)cc3)c3ccc(-c4ccccc4-c4ccccc4)cc3)ccc2-c2cc3cc4c(cc3cc21)CCCC4]

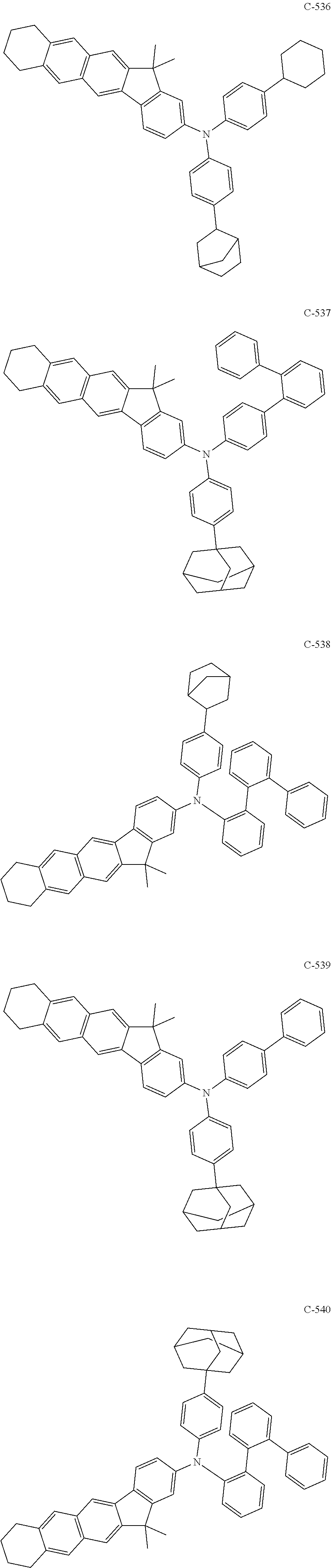 [SMILES: CC1(C)c2cc(N(c3ccc(C4CCCCC4)cc3)c3ccc(C4CC5CCC4C5)cc3)ccc2-c2cc3cc4c(cc3cc21)CCCC4 ; CC1(C)c2cc(N(c3ccc(-c4ccccc4-c4ccccc4)cc3)c3ccc(C45CC6CC(CC(C6)C4)C5)cc3)ccc2-c2cc3cc4c(cc3cc21)CCCC4 ; CC1(C)c2cc(N(c3ccc(C4CC5CCC4C5)cc3)c3ccccc3-c3ccccc3-c3ccccc3)ccc2-c2cc3cc4c(cc3cc21)CCCC4 ; CC1(C)c2cc(N(c3ccc(-c4ccccc4)cc3)c3ccc(C45CC6CC(CC(C6)C4)C5)cc3)ccc2-c2cc3cc4c(cc3cc21)CCCC4 ; CC1(C)c2cc(N(c3ccc(C45CC6CC(CC(C6)C4)C5)cc3)c3ccccc3-c3ccccc3-c3ccccc3)ccc2-c2cc3cc4c(cc3cc21)CCCC4]

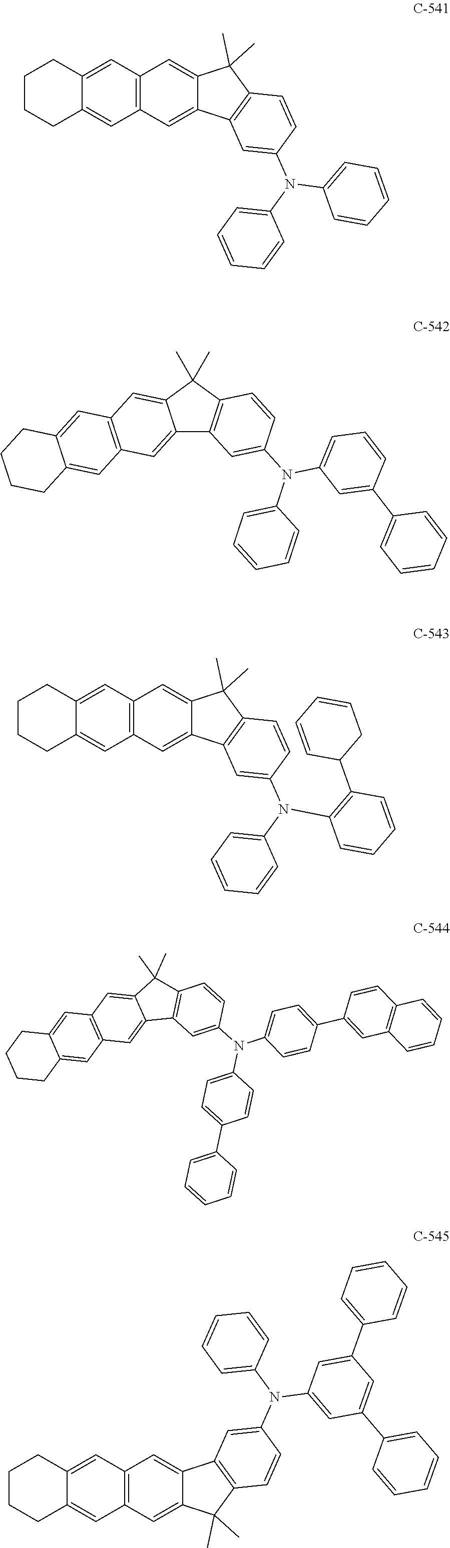 [SMILES: CC1(C)c2ccc(N(c3ccccc3)c3ccccc3)cc2-c2cc3cc4c(cc3cc21)CCCC4 ; CC1(C)c2ccc(N(c3ccccc3)c3cccc(-c4ccccc4)c3)cc2-c2cc3cc4c(cc3cc21)CCCC4 ; CC1(C)c2ccc(N(c3ccccc3)c3ccccc3C3C=CC=CC3)cc2-c2cc3cc4c(cc3cc21)CCCC4 ; CC1(C)c2ccc(N(c3ccc(-c4ccccc4)cc3)c3ccc(-c4ccc5ccccc5c4)cc3)cc2-c2cc3cc4c(cc3cc21)CCCC4 ; CC1(C)c2ccc(N(c3ccccc3)c3cc(-c4ccccc4)cc(-c4ccccc4)c3)cc2-c2cc3cc4c(cc3cc21)CCCC4]

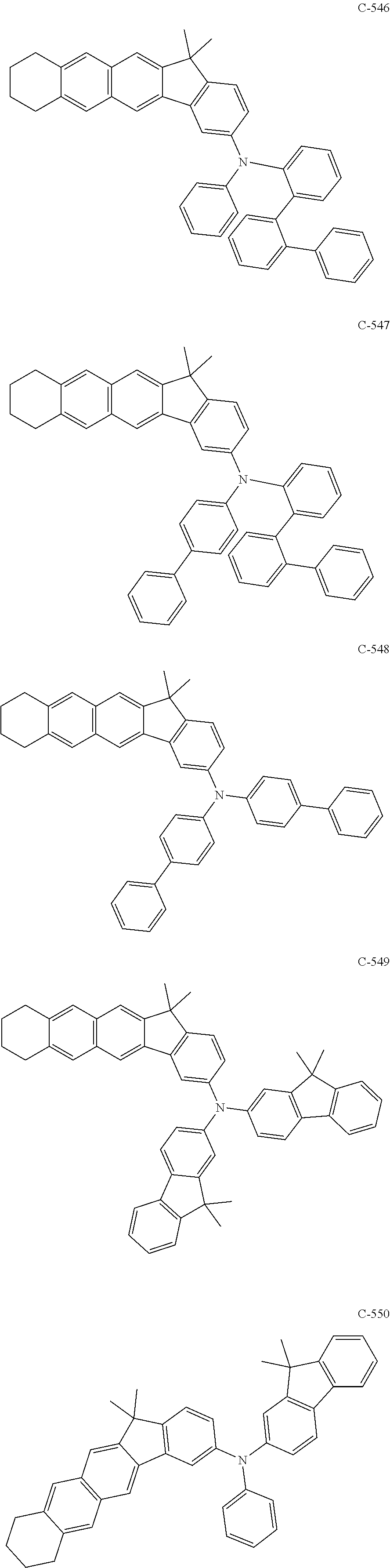 [SMILES: CC1(C)c2ccc(N(c3ccccc3)c3ccccc3-c3ccccc3-c3ccccc3)cc2-c2cc3cc4c(cc3cc21)CCCC4 ; CC1(C)c2ccc(N(c3ccc(-c4ccccc4)cc3)c3ccccc3-c3ccccc3-c3ccccc3)cc2-c2cc3cc4c(cc3cc21)CCCC4 ; CC1(C)c2ccc(N(c3ccc(-c4ccccc4)cc3)c3ccc(-c4ccccc4)cc3)cc2-c2cc3cc4c(cc3cc21)CCCC4 ; CC1(C)c2ccccc2-c2ccc(N(c3ccc4c(c3)-c3cc5cc6c(cc5cc3C4(C)C)CCCC6)c3ccc4c(c3)C(C)(C)c3ccccc3-4)cc21 ; CC1(C)c2ccccc2-c2ccc(N(c3ccccc3)c3ccc4c(c3)-c3cc5cc6c(cc5cc3C4(C)C)CCCC6)cc21]

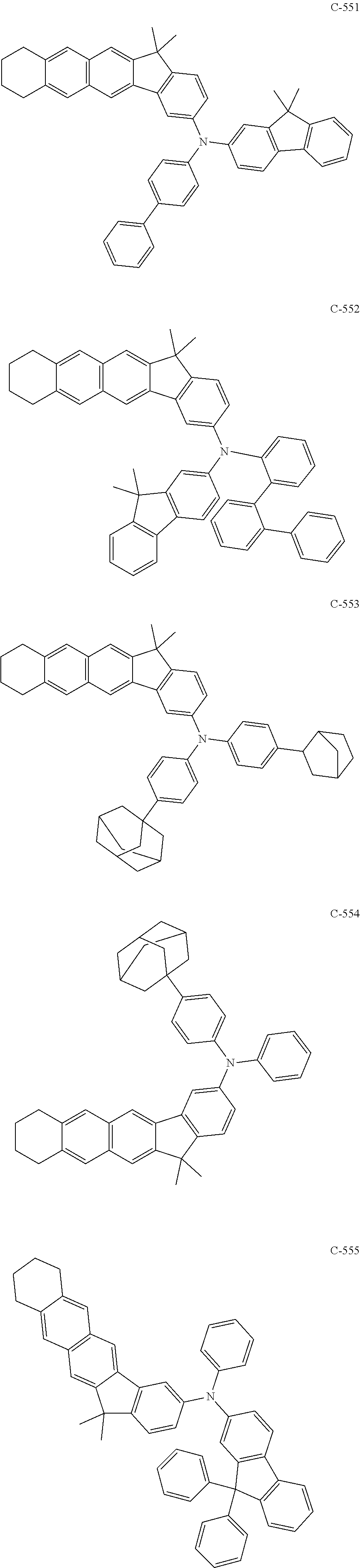 [SMILES: CC1(C)c2ccccc2-c2ccc(N(c3ccc(-c4ccccc4)cc3)c3ccc4c(c3)-c3cc5cc6c(cc5cc3C4(C)C)CCCC6)cc21 ; CC1(C)c2ccccc2-c2ccc(N(c3ccc4c(c3)-c3cc5cc6c(cc5cc3C4(C)C)CCCC6)c3ccccc3-c3ccccc3-c3ccccc3)cc21 ; CC1(C)c2ccc(N(c3ccc(C4CC5CCC4C5)cc3)c3ccc(C45CC6CC(CC(C6)C4)C5)cc3)cc2-c2cc3cc4c(cc3cc21)CCCC4 ; CC1(C)c2ccc(N(c3ccccc3)c3ccc(C45CC6CC(CC(C6)C4)C5)cc3)cc2-c2cc3cc4c(cc3cc21)CCCC4 ; CC1(C)c2ccc(N(c3ccccc3)c3ccc4c(c3)C(c3ccccc3)(c3ccccc3)c3ccccc3-4)cc2-c2cc3cc4c(cc3cc21)CCCC4]

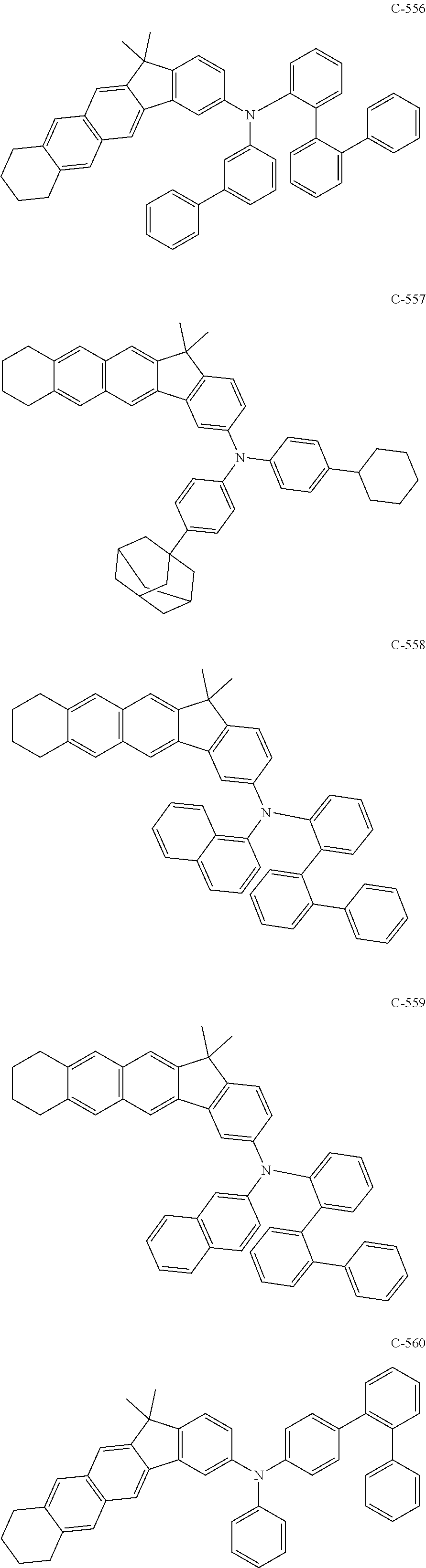 [SMILES: CC1(C)c2ccc(N(c3cccc(-c4ccccc4)c3)c3ccccc3-c3ccccc3-c3ccccc3)cc2-c2cc3cc4c(cc3cc21)CCCC4 ; CC1(C)c2ccc(N(c3ccc(C4CCCCC4)cc3)c3ccc(C45CC6CC(CC(C6)C4)C5)cc3)cc2-c2cc3cc4c(cc3cc21)CCCC4 ; CC1(C)c2ccc(N(c3ccccc3-c3ccccc3-c3ccccc3)c3cccc4ccccc34)cc2-c2cc3cc4c(cc3cc21)CCCC4 ; CC1(C)c2ccc(N(c3ccc4ccccc4c3)c3ccccc3-c3ccccc3-c3ccccc3)cc2-c2cc3cc4c(cc3cc21)CCCC4 ; CC1(C)c2ccc(N(c3ccccc3)c3ccc(-c4ccccc4-c4ccccc4)cc3)cc2-c2cc3cc4c(cc3cc21)CCCC4]

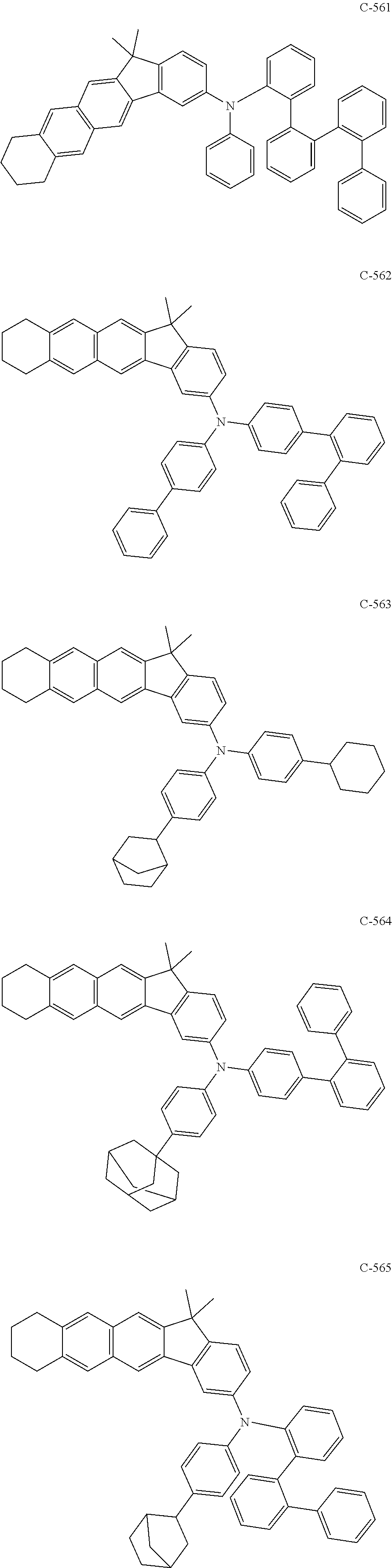 [SMILES: CC1(C)c2ccc(N(c3ccccc3)c3ccccc3-c3ccccc3-c3ccccc3-c3ccccc3)cc2-c2cc3cc4c(cc3cc21)CCCC4 ; CC1(C)c2ccc(N(c3ccc(-c4ccccc4)cc3)c3ccc(-c4ccccc4-c4ccccc4)cc3)cc2-c2cc3cc4c(cc3cc21)CCCC4 ; CC1(C)c2ccc(N(c3ccc(C4CCCCC4)cc3)c3ccc(C4CC5CCC4C5)cc3)cc2-c2cc3cc4c(cc3cc21)CCCC4 ; CC1(C)c2ccc(N(c3ccc(-c4ccccc4-c4ccccc4)cc3)c3ccc(C45CC6CC(CC(C6)C4)C5)cc3)cc2-c2cc3cc4c(cc3cc21)CCCC4 ; CC1(C)c2ccc(N(c3ccc(C4CC5CCC4C5)cc3)c3ccccc3-c3ccccc3-c3ccccc3)cc2-c2cc3cc4c(cc3cc21)CCCC4]

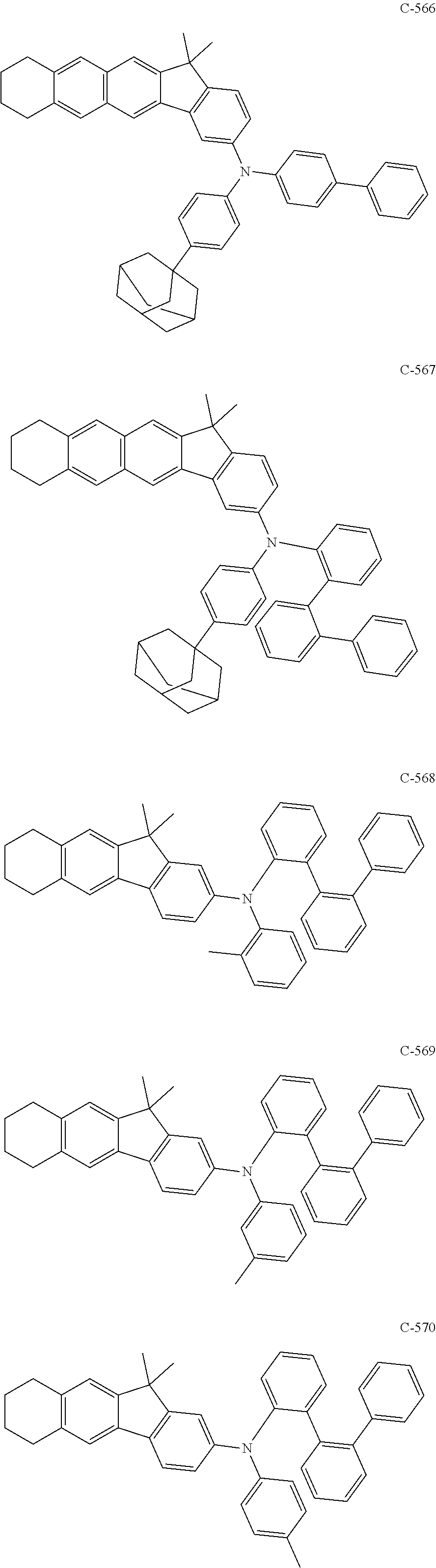 [SMILES: CC1(C)c2ccc(N(c3ccc(-c4ccccc4)cc3)c3ccc(C45CC6CC(CC(C6)C4)C5)cc3)cc2-c2cc3cc4c(cc3cc21)CCCC4 ; CC1(C)c2ccc(N(c3ccc(C45CC6CC(CC(C6)C4)C5)cc3)c3ccccc3-c3ccccc3-c3ccccc3)cc2-c2cc3cc4c(cc3cc21)CCCC4 ; Cc1ccccc1N(c1ccc2c(c1)C(C)(C)c1cc3c(cc1-2)CCCC3)c1ccccc1-c1ccccc1-c1ccccc1 ; Cc1cccc(N(c2ccc3c(c2)C(C)(C)c2cc4c(cc2-3)CCCC4)c2ccccc2-c2ccccc2-c2ccccc2)c1 ; Cc1ccc(N(c2ccc3c(c2)C(C)(C)c2cc4c(cc2-3)CCCC4)c2ccccc2-c2ccccc2-c2ccccc2)cc1]

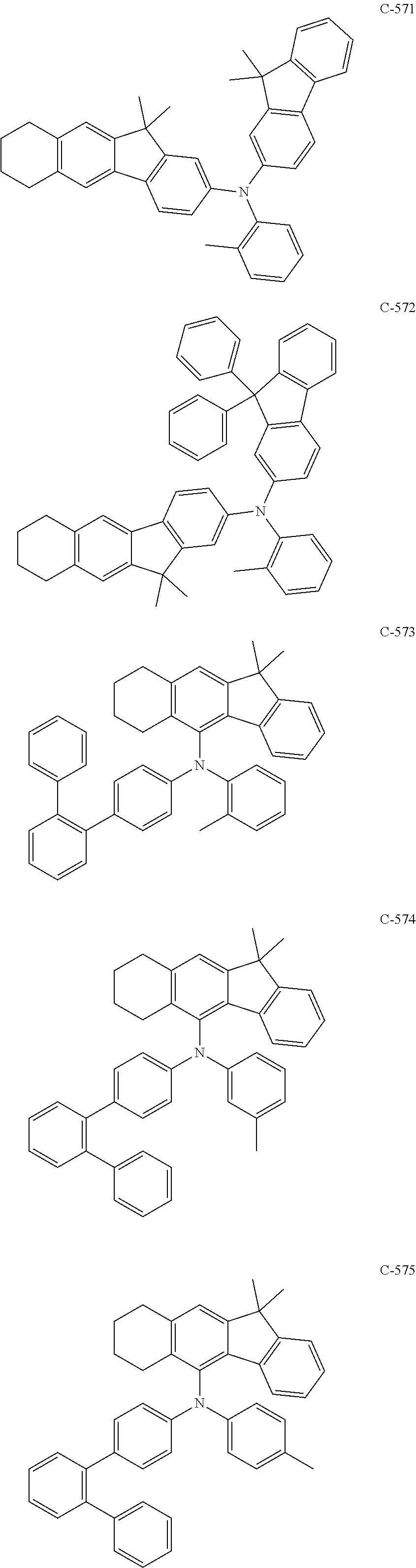 [SMILES: Cc1ccccc1N(c1ccc2c(c1)C(C)(C)c1ccccc1-2)c1ccc2c(c1)C(C)(C)c1cc3c(cc1-2)CCCC3 ; Cc1ccccc1N(c1ccc2c(c1)C(C)(C)c1cc3c(cc1-2)CCCC3)c1ccc2c(c1)C(c1ccccc1)(c1ccccc1)c1ccccc1-2 ; Cc1ccccc1N(c1ccc(-c2ccccc2-c2ccccc2)cc1)c1c2c(cc3c1-c1ccccc1C3(C)C)CCCC2 ; Cc1cccc(N(c2ccc(-c3ccccc3-c3ccccc3)cc2)c2c3c(cc4c2-c2ccccc2C4(C)C)CCCC3)c1 ; Cc1ccc(N(c2ccc(-c3ccccc3-c3ccccc3)cc2)c2c3c(cc4c2-c2ccccc2C4(C)C)CCCC3)cc1]

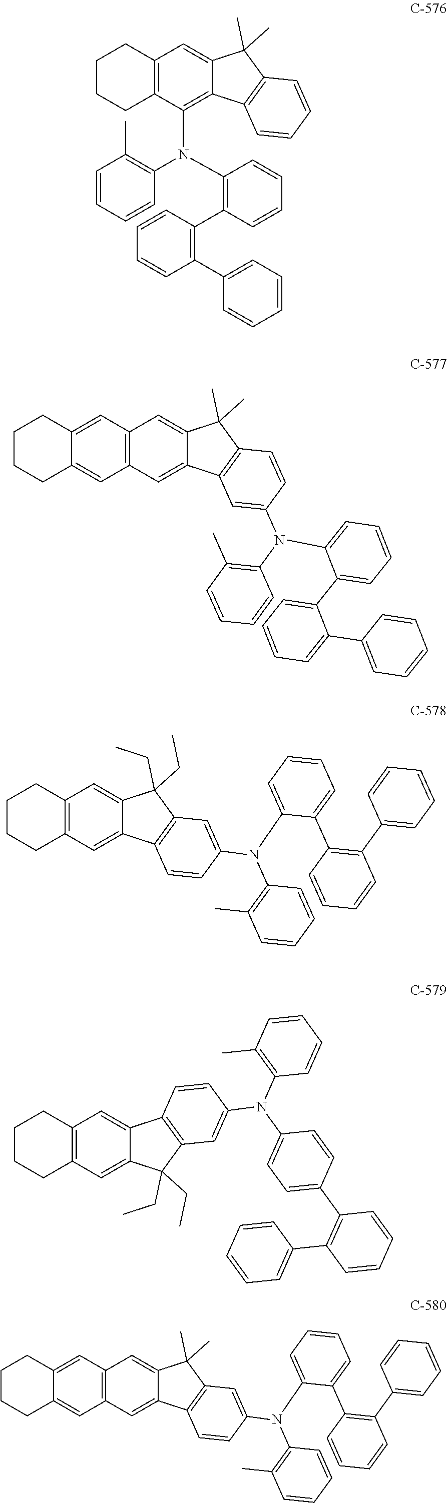 [SMILES: Cc1ccccc1N(c1ccccc1-c1ccccc1-c1ccccc1)c1c2c(cc3c1-c1ccccc1C3(C)C)CCCC2 ; Cc1ccccc1N(c1ccc2c(c1)-c1cc3cc4c(cc3cc1C2(C)C)CCCC4)c1ccccc1-c1ccccc1-c1ccccc1 ; CCC1(CC)c2cc(N(c3ccccc3C)c3ccccc3-c3ccccc3-c3ccccc3)ccc2-c2cc3c(cc21)CCCC3 ; CCC1(CC)c2cc(N(c3ccc(-c4ccccc4-c4ccccc4)cc3)c3ccccc3C)ccc2-c2cc3c(cc21)CCCC3 ; Cc1ccccc1N(c1ccc2c(c1)C(C)(C)c1cc3cc4c(cc3cc1-2)CCCC4)c1ccccc1-c1ccccc1-c1ccccc1]

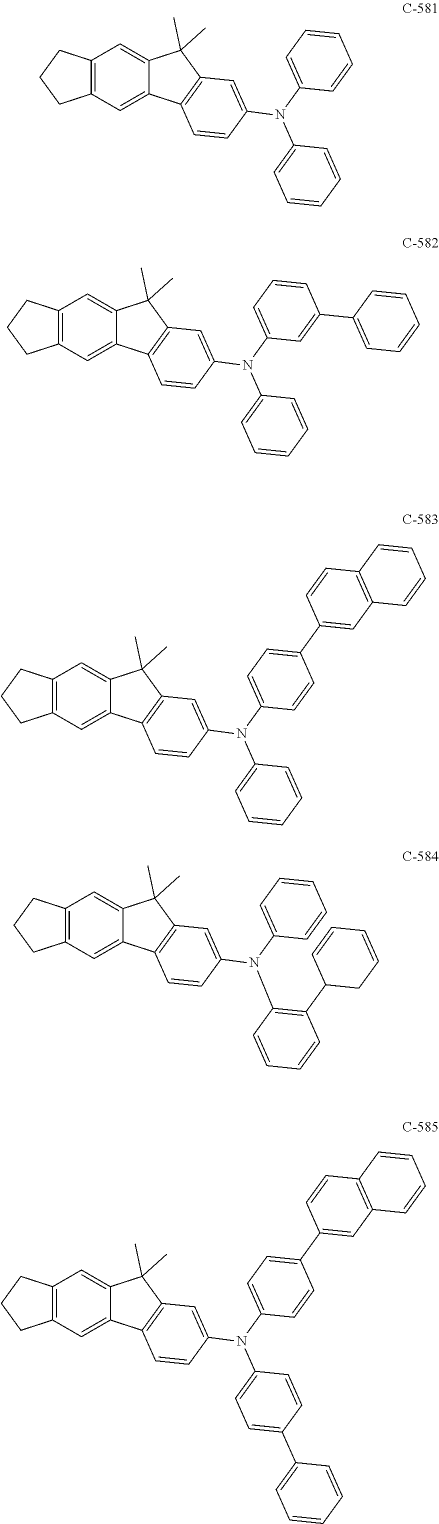 [SMILES: CC1(C)c2cc(N(c3ccccc3)c3ccccc3)ccc2-c2cc3c(cc21)CCC3 ; CC1(C)c2cc(N(c3ccccc3)c3cccc(-c4ccccc4)c3)ccc2-c2cc3c(cc21)CCC3 ; CC1(C)c2cc(N(c3ccccc3)c3ccc(-c4ccc5ccccc5c4)cc3)ccc2-c2cc3c(cc21)CCC3 ; CC1(C)c2cc(N(c3ccccc3)c3ccccc3C3C=CC=CC3)ccc2-c2cc3c(cc21)CCC3 ; CC1(C)c2cc(N(c3ccc(-c4ccccc4)cc3)c3ccc(-c4ccc5ccccc5c4)cc3)ccc2-c2cc3c(cc21)CCC3]

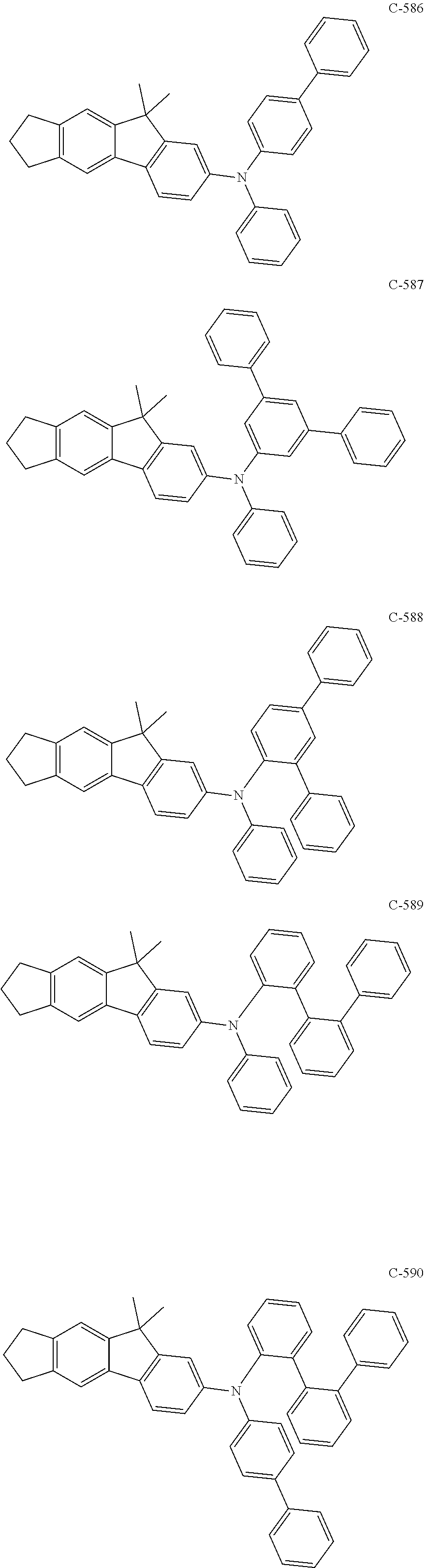 [SMILES: CC1(C)c2cc(N(c3ccccc3)c3ccc(-c4ccccc4)cc3)ccc2-c2cc3c(cc21)CCC3 ; CC1(C)c2cc(N(c3ccccc3)c3cc(-c4ccccc4)cc(-c4ccccc4)c3)ccc2-c2cc3c(cc21)CCC3 ; CC1(C)c2cc(N(c3ccccc3)c3ccc(-c4ccccc4)cc3-c3ccccc3)ccc2-c2cc3c(cc21)CCC3 ; CC1(C)c2cc(N(c3ccccc3)c3ccccc3-c3ccccc3-c3ccccc3)ccc2-c2cc3c(cc21)CCC3 ; CC1(C)c2cc(N(c3ccc(-c4ccccc4)cc3)c3ccccc3-c3ccccc3-c3ccccc3)ccc2-c2cc3c(cc21)CCC3]

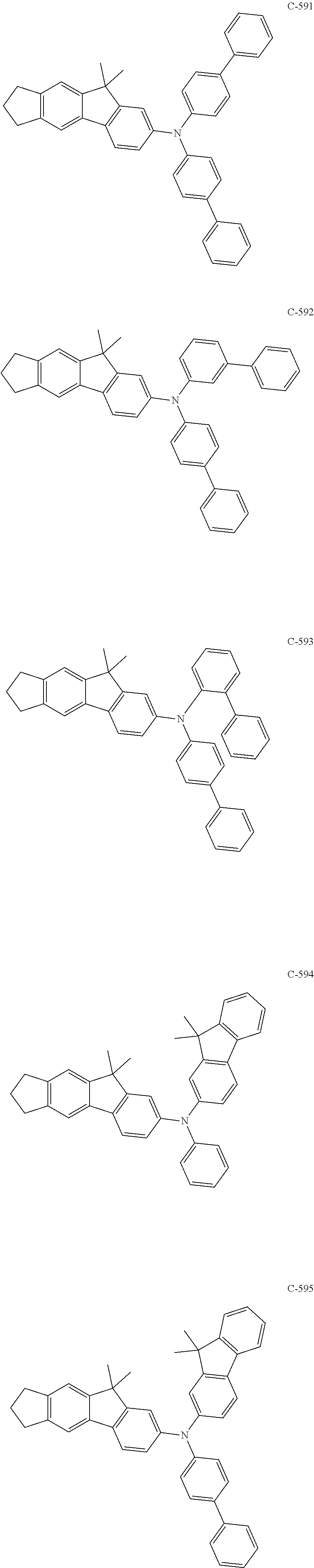 [SMILES: CC1(C)c2cc(N(c3ccc(-c4ccccc4)cc3)c3ccc(-c4ccccc4)cc3)ccc2-c2cc3c(cc21)CCC3 ; CC1(C)c2cc(N(c3ccc(-c4ccccc4)cc3)c3cccc(-c4ccccc4)c3)ccc2-c2cc3c(cc21)CCC3 ; CC1(C)c2cc(N(c3ccc(-c4ccccc4)cc3)c3ccccc3-c3ccccc3)ccc2-c2cc3c(cc21)CCC3 ; CC1(C)c2ccccc2-c2ccc(N(c3ccccc3)c3ccc4c(c3)C(C)(C)c3cc5c(cc3-4)CCC5)cc21 ; CC1(C)c2ccccc2-c2ccc(N(c3ccc(-c4ccccc4)cc3)c3ccc4c(c3)C(C)(C)c3cc5c(cc3-4)CCC5)cc21]

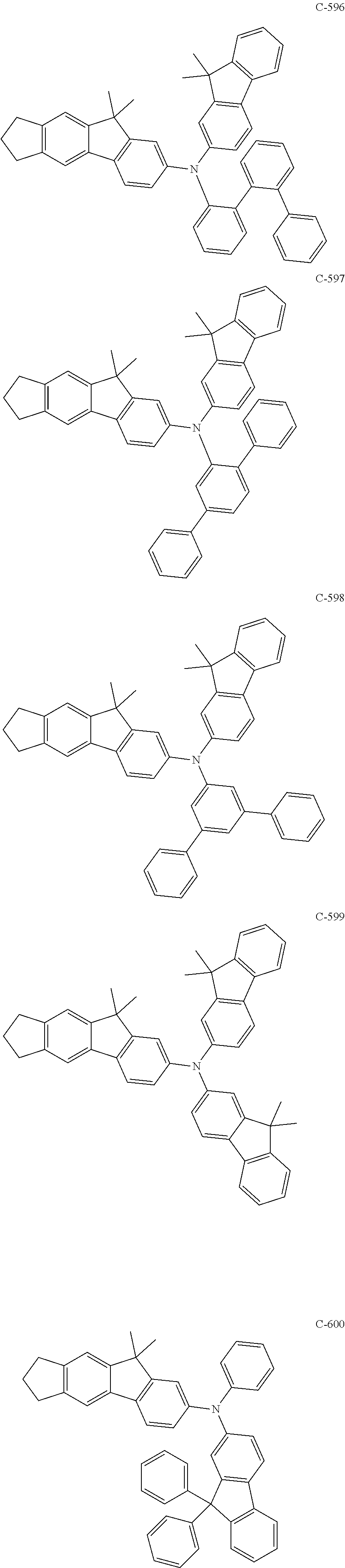

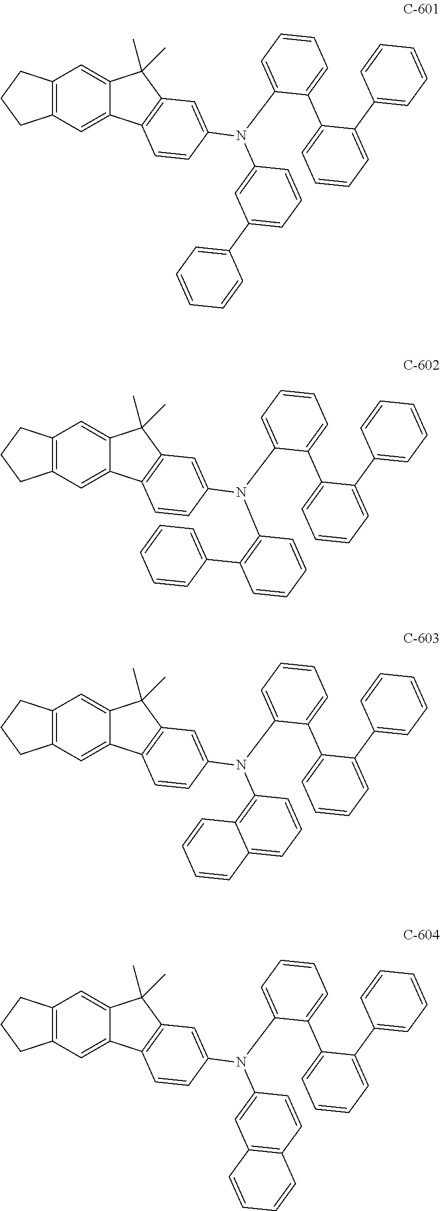 [SMILES: CC1(C)c2cc(N(c3cccc(-c4ccccc4)c3)c3ccccc3-c3ccccc3-c3ccccc3)ccc2-c2cc3c(cc21)CCC3 ; CC1(C)c2cc(N(c3ccccc3-c3ccccc3)c3ccccc3-c3ccccc3-c3ccccc3)ccc2-c2cc3c(cc21)CCC3 ; CC1(C)c2cc(N(c3ccccc3-c3ccccc3-c3ccccc3)c3cccc4ccccc34)ccc2-c2cc3c(cc21)CCC3 ; CC1(C)c2cc(N(c3ccc4ccccc4c3)c3ccccc3-c3ccccc3-c3ccccc3)ccc2-c2cc3c(cc21)CCC3]

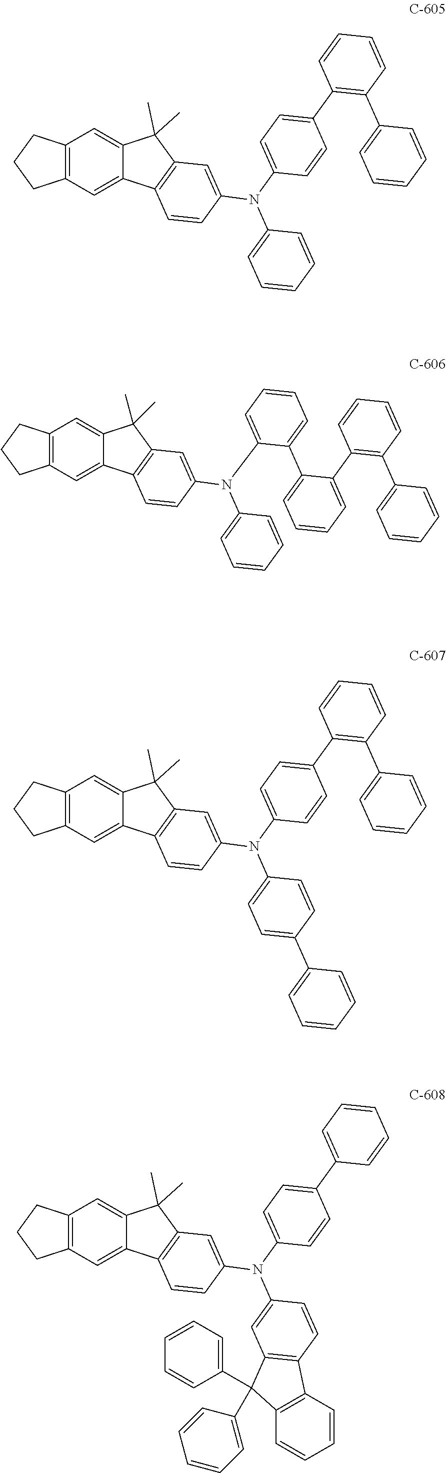 [SMILES: CC1(C)c2cc(N(c3ccccc3)c3ccc(-c4ccccc4-c4ccccc4)cc3)ccc2-c2cc3c(cc21)CCC3 ; CC1(C)c2cc(N(c3ccccc3)c3ccccc3-c3ccccc3-c3ccccc3-c3ccccc3)ccc2-c2cc3c(cc21)CCC3 ; CC1(C)c2cc(N(c3ccc(-c4ccccc4)cc3)c3ccc(-c4ccccc4-c4ccccc4)cc3)ccc2-c2cc3c(cc21)CCC3 ; CC1(C)c2cc(N(c3ccc(-c4ccccc4)cc3)c3ccc4c(c3)C(c3ccccc3)(c3ccccc3)c3ccccc3-4)ccc2-c2cc3c(cc21)CCC3]

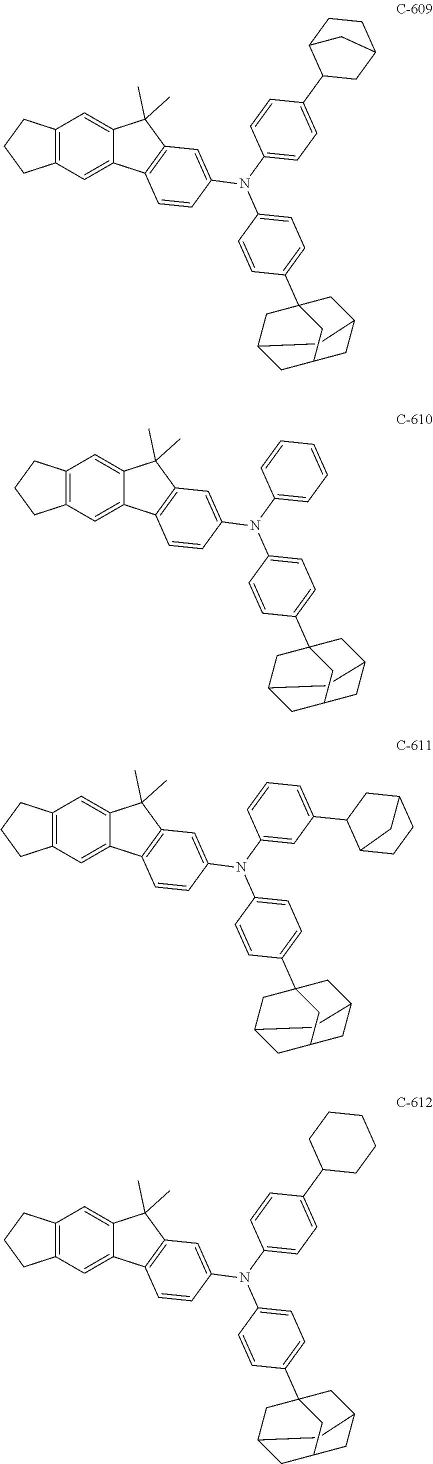 [SMILES: CC1(C)c2cc(N(c3ccc(C4CC5CCC4C5)cc3)c3ccc(C45CC6CC(CC(C6)C4)C5)cc3)ccc2-c2cc3c(cc21)CCC3 ; CC1(C)c2cc(N(c3ccccc3)c3ccc(C45CC6CC(CC(C6)C4)C5)cc3)ccc2-c2cc3c(cc21)CCC3 ; CC1(C)c2cc(N(c3ccc(C45CC6CC(CC(C6)C4)C5)cc3)c3cccc(C4CC5CCC4C5)c3)ccc2-c2cc3c(cc21)CCC3 ; CC1(C)c2cc(N(c3ccc(C4CCCCC4)cc3)c3ccc(C45CC6CC(CC(C6)C4)C5)cc3)ccc2-c2cc3c(cc21)CCC3]

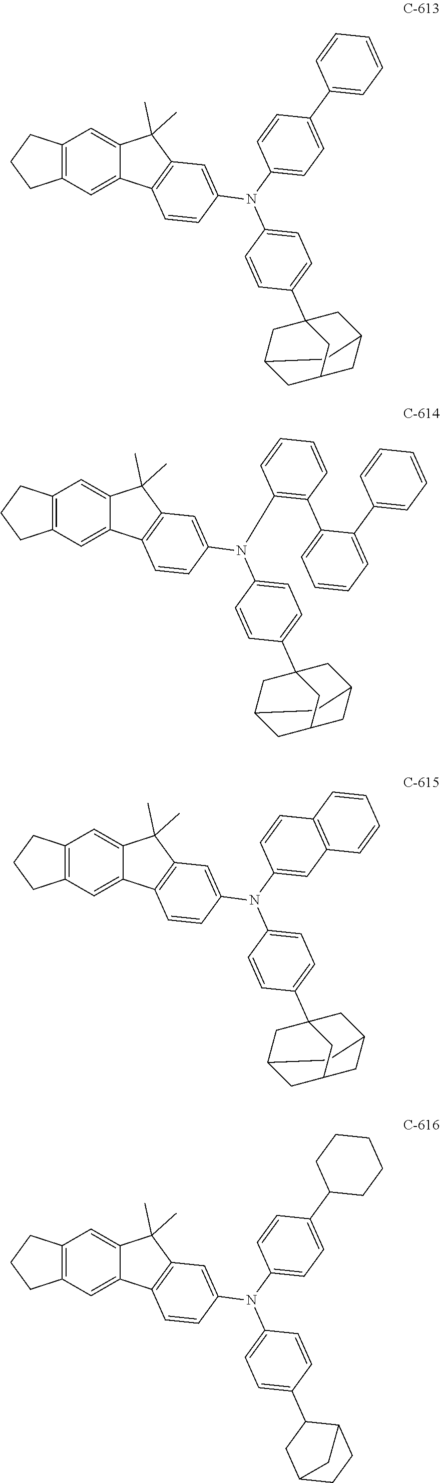 [SMILES: CC1(C)c2cc(N(c3ccc(-c4ccccc4)cc3)c3ccc(C45CC6CC(CC(C6)C4)C5)cc3)ccc2-c2cc3c(cc21)CCC3 ; CC1(C)c2cc(N(c3ccc(C45CC6CC(CC(C6)C4)C5)cc3)c3ccccc3-c3ccccc3-c3ccccc3)ccc2-c2cc3c(cc21)CCC3 ; CC1(C)c2cc(N(c3ccc(C45CC6CC(CC(C6)C4)C5)cc3)c3ccc4ccccc4c3)ccc2-c2cc3c(cc21)CCC3 ; CC1(C)c2cc(N(c3ccc(C4CCCCC4)cc3)c3ccc(C4CC5CCC4C5)cc3)ccc2-c2cc3c(cc21)CCC3]

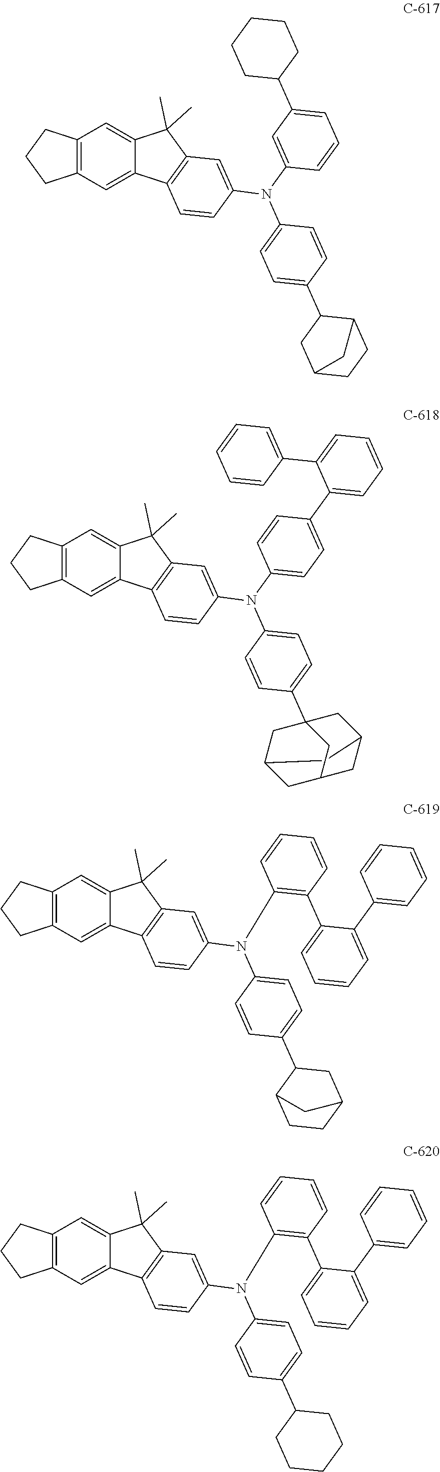 [SMILES: CC1(C)c2cc(N(c3ccc(C4CC5CCC4C5)cc3)c3cccc(C4CCCCC4)c3)ccc2-c2cc3c(cc21)CCC3 ; CC1(C)c2cc(N(c3ccc(-c4ccccc4-c4ccccc4)cc3)c3ccc(C45CC6CC(CC(C6)C4)C5)cc3)ccc2-c2cc3c(cc21)CCC3 ; CC1(C)c2cc(N(c3ccc(C4CC5CCC4C5)cc3)c3ccccc3-c3ccccc3-c3ccccc3)ccc2-c2cc3c(cc21)CCC3 ; CC1(C)c2cc(N(c3ccc(C4CCCCC4)cc3)c3ccccc3-c3ccccc3-c3ccccc3)ccc2-c2cc3c(cc21)CCC3]

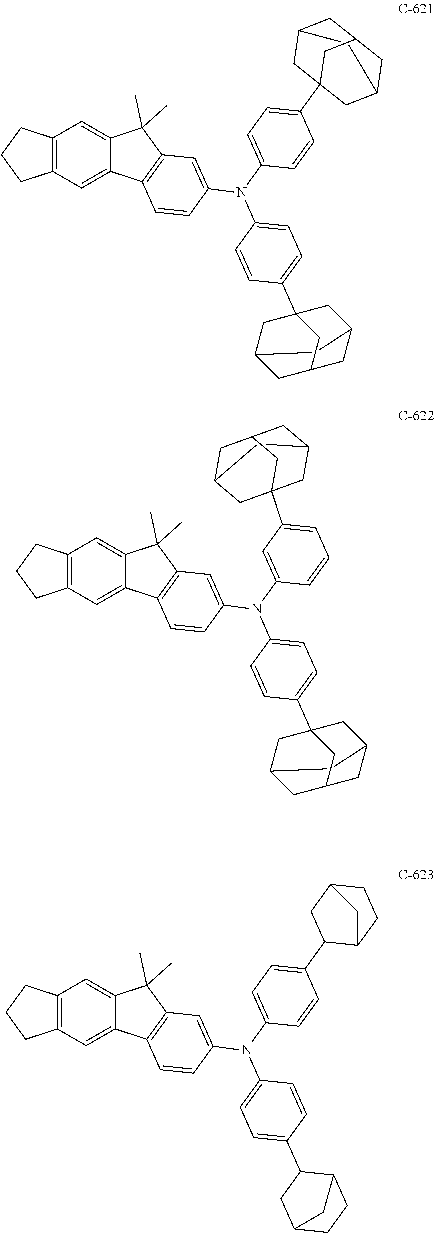 [SMILES: CC1(C)c2cc(N(c3ccc(C45CC6CC(CC(C6)C4)C5)cc3)c3ccc(C45CC6CC(CC(C6)C4)C5)cc3)ccc2-c2cc3c(cc21)CCC3 ; CC1(C)c2cc(N(c3ccc(C45CC6CC(CC(C6)C4)C5)cc3)c3cccc(C45CC6CC(CC(C6)C4)C5)c3)ccc2-c2cc3c(cc21)CCC3 ; CC1(C)c2cc(N(c3ccc(C4CC5CCC4C5)cc3)c3ccc(C4CC5CCC4C5)cc3)ccc2-c2cc3c(cc21)CCC3]

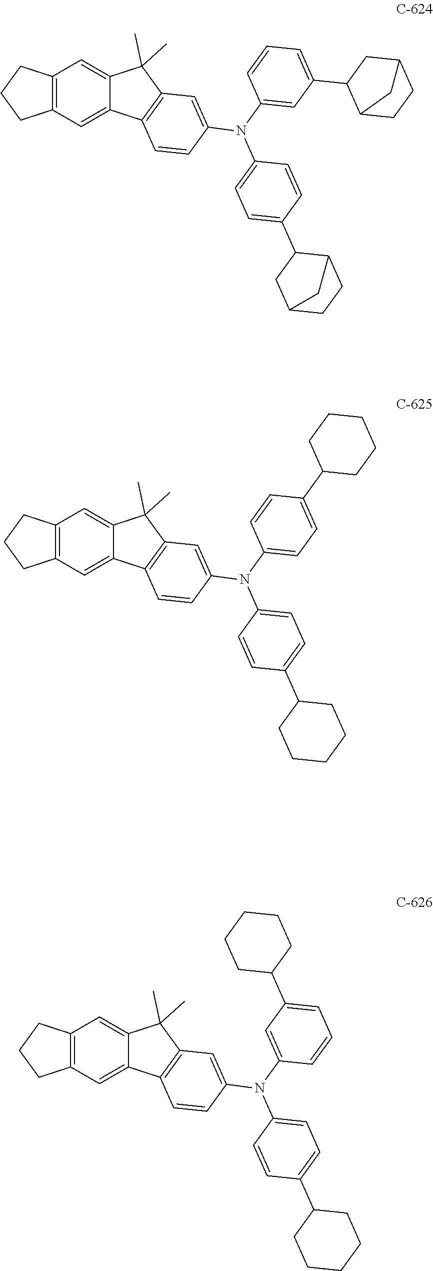 [SMILES: CC1(C)c2cc(N(c3ccc(C4CC5CCC4C5)cc3)c3cccc(C4CC5CCC4C5)c3)ccc2-c2cc3c(cc21)CCC3 ; CC1(C)c2cc(N(c3ccc(C4CCCCC4)cc3)c3ccc(C4CCCCC4)cc3)ccc2-c2cc3c(cc21)CCC3 ; CC1(C)c2cc(N(c3ccc(C4CCCCC4)cc3)c3cccc(C4CCCCC4)c3)ccc2-c2cc3c(cc21)CCC3]

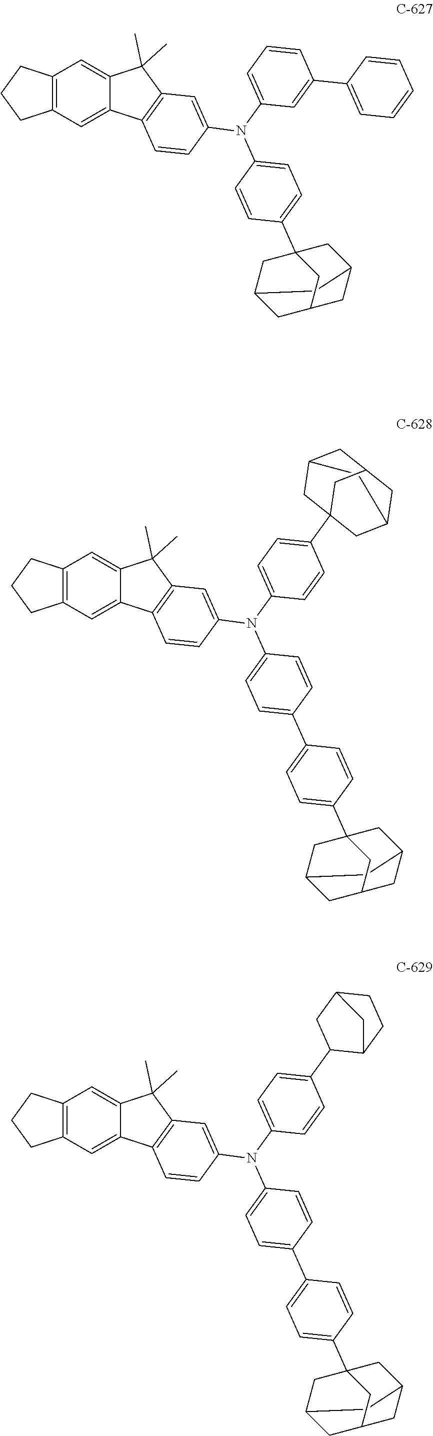 [SMILES: CC1(C)c2cc(N(c3ccc(C45CC6CC(CC(C6)C4)C5)cc3)c3cccc(-c4ccccc4)c3)ccc2-c2cc3c(cc21)CCC3 ; CC1(C)c2cc(N(c3ccc(-c4ccc(C56CC7CC(CC(C7)C5)C6)cc4)cc3)c3ccc(C45CC6CC(CC(C6)C4)C5)cc3)ccc2-c2cc3c(cc21)CCC3 ; CC1(C)c2cc(N(c3ccc(-c4ccc(C56CC7CC(CC(C7)C5)C6)cc4)cc3)c3ccc(C4CC5CCC4C5)cc3)ccc2-c2cc3c(cc21)CCC3]

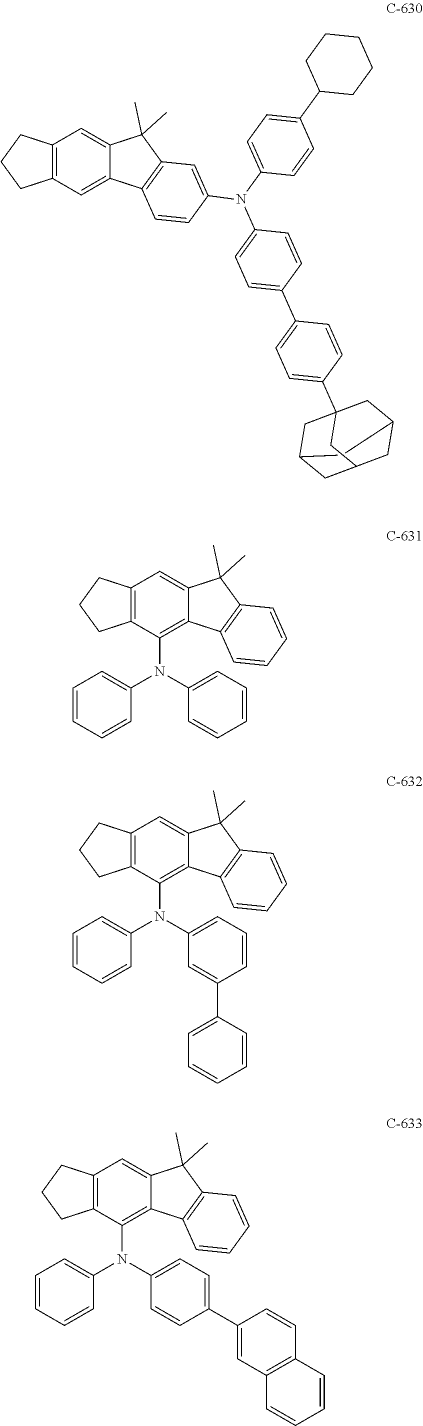 [SMILES: CC1(C)c2cc(N(c3ccc(-c4ccc(C56CC7CC(CC(C7)C5)C6)cc4)cc3)c3ccc(C4CCCCC4)cc3)ccc2-c2cc3c(cc21)CCC3 ; CC1(C)c2ccccc2-c2c1cc1c(c2N(c2ccccc2)c2ccccc2)CCC1 ; CC1(C)c2ccccc2-c2c1cc1c(c2N(c2ccccc2)c2cccc(-c3ccccc3)c2)CCC1 ; CC1(C)c2ccccc2-c2c1cc1c(c2N(c2ccccc2)c2ccc(-c3ccc4ccccc4c3)cc2)CCC1]

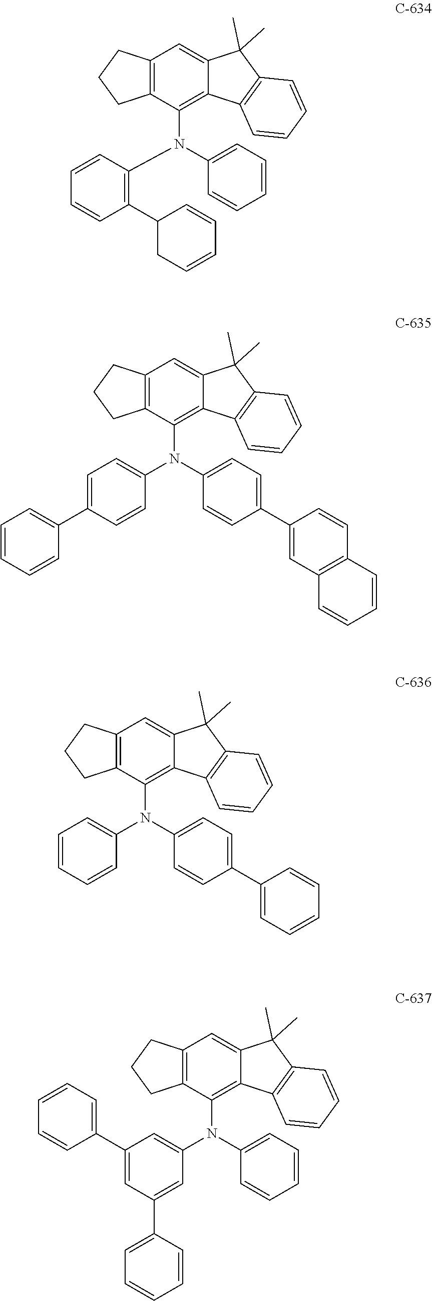 [SMILES: CC1(C)c2ccccc2-c2c1cc1c(c2N(c2ccccc2)c2ccccc2C2C=CC=CC2)CCC1 ; CC1(C)c2ccccc2-c2c1cc1c(c2N(c2ccc(-c3ccccc3)cc2)c2ccc(-c3ccc4ccccc4c3)cc2)CCC1 ; CC1(C)c2ccccc2-c2c1cc1c(c2N(c2ccccc2)c2ccc(-c3ccccc3)cc2)CCC1 ; CC1(C)c2ccccc2-c2c1cc1c(c2N(c2ccccc2)c2cc(-c3ccccc3)cc(-c3ccccc3)c2)CCC1]

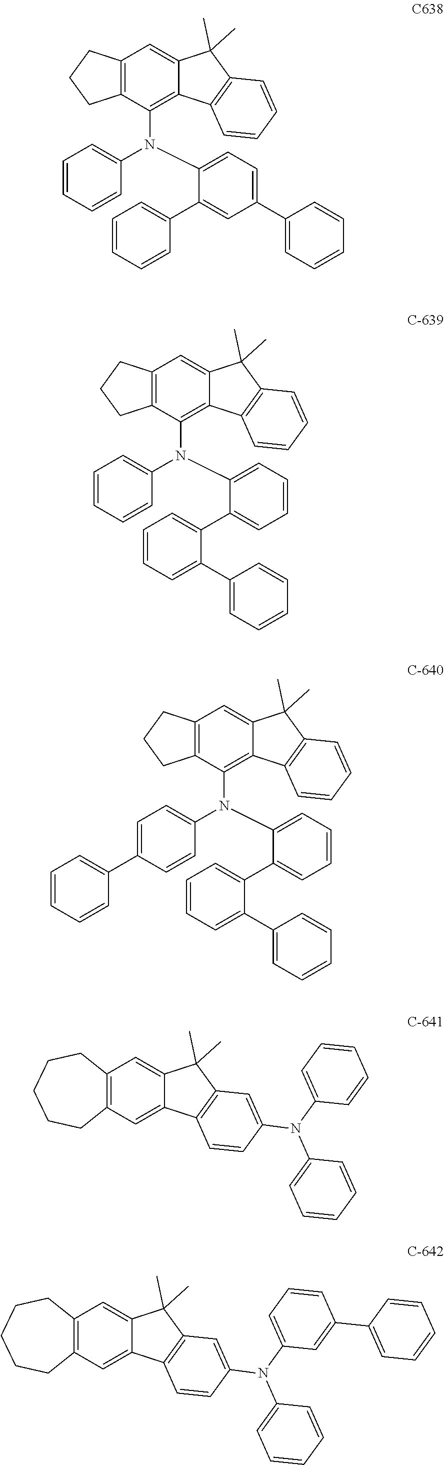 [SMILES: CC1(C)c2ccccc2-c2c1cc1c(c2N(c2ccccc2)c2ccc(-c3ccccc3)cc2-c2ccccc2)CCC1 ; CC1(C)c2ccccc2-c2c1cc1c(c2N(c2ccccc2)c2ccccc2-c2ccccc2-c2ccccc2)CCC1 ; CC1(C)c2ccccc2-c2c1cc1c(c2N(c2ccc(-c3ccccc3)cc2)c2ccccc2-c2ccccc2-c2ccccc2)CCC1 ; CC1(C)c2cc(N(c3ccccc3)c3ccccc3)ccc2-c2cc3c(cc21)CCCCC3 ; CC1(C)c2cc(N(c3ccccc3)c3cccc(-c4ccccc4)c3)ccc2-c2cc3c(cc21)CCCCC3]

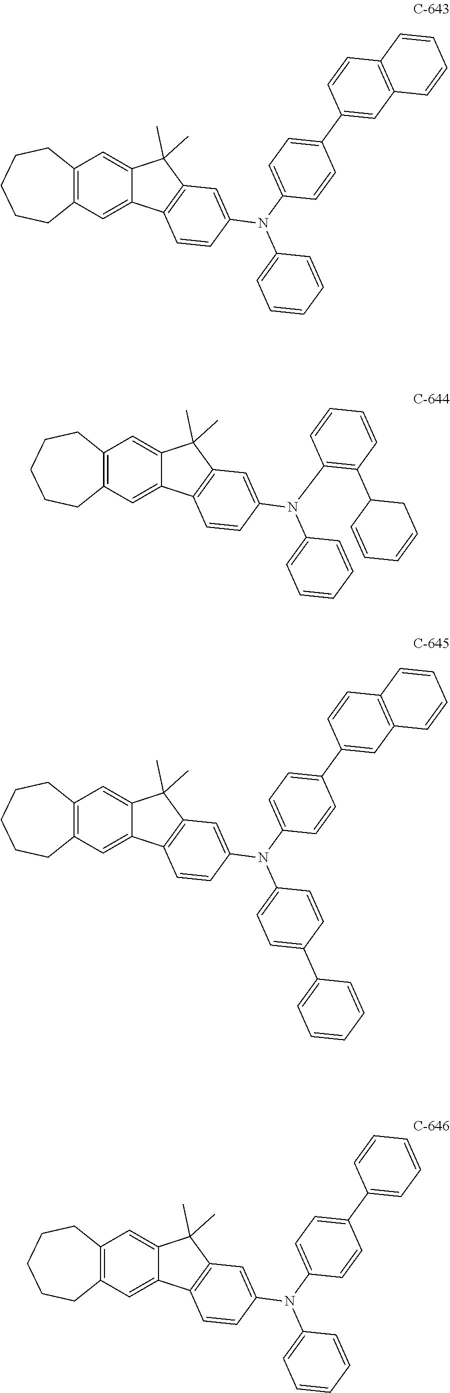 [SMILES: CC1(C)c2cc(N(c3ccccc3)c3ccc(-c4ccc5ccccc5c4)cc3)ccc2-c2cc3c(cc21)CCCCC3 ; CC1(C)c2cc(N(c3ccccc3)c3ccccc3C3C=CC=CC3)ccc2-c2cc3c(cc21)CCCCC3 ; CC1(C)c2cc(N(c3ccc(-c4ccccc4)cc3)c3ccc(-c4ccc5ccccc5c4)cc3)ccc2-c2cc3c(cc21)CCCCC3 ; CC1(C)c2cc(N(c3ccccc3)c3ccc(-c4ccccc4)cc3)ccc2-c2cc3c(cc21)CCCCC3]

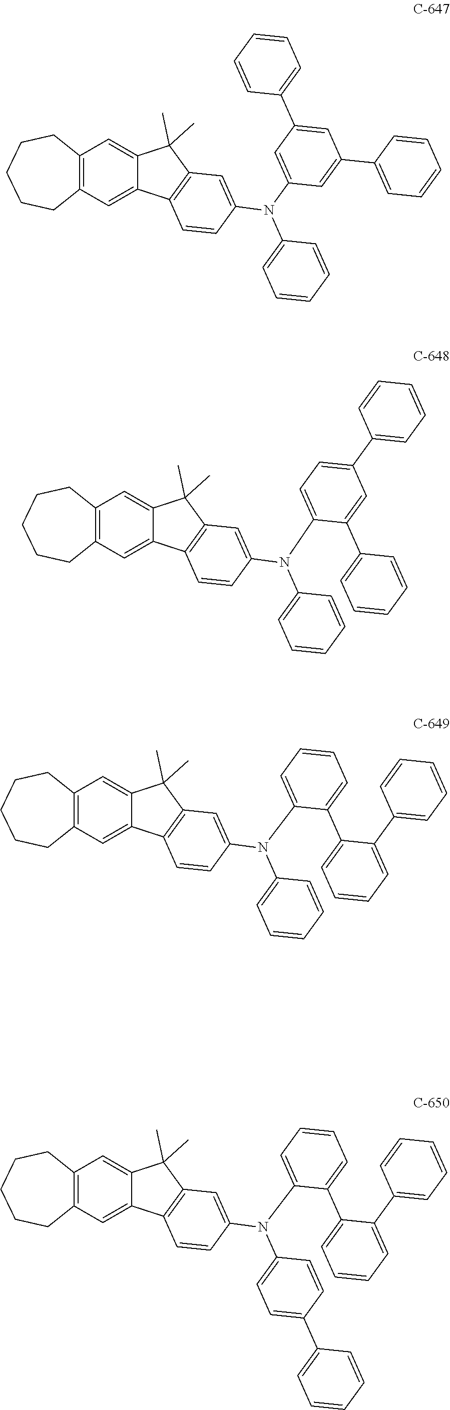 [SMILES: CC1(C)c2cc(N(c3ccccc3)c3cc(-c4ccccc4)cc(-c4ccccc4)c3)ccc2-c2cc3c(cc21)CCCCC3 ; CC1(C)c2cc(N(c3ccccc3)c3ccc(-c4ccccc4)cc3-c3ccccc3)ccc2-c2cc3c(cc21)CCCCC3 ; CC1(C)c2cc(N(c3ccccc3)c3ccccc3-c3ccccc3-c3ccccc3)ccc2-c2cc3c(cc21)CCCCC3 ; CC1(C)c2cc(N(c3ccc(-c4ccccc4)cc3)c3ccccc3-c3ccccc3-c3ccccc3)ccc2-c2cc3c(cc21)CCCCC3]

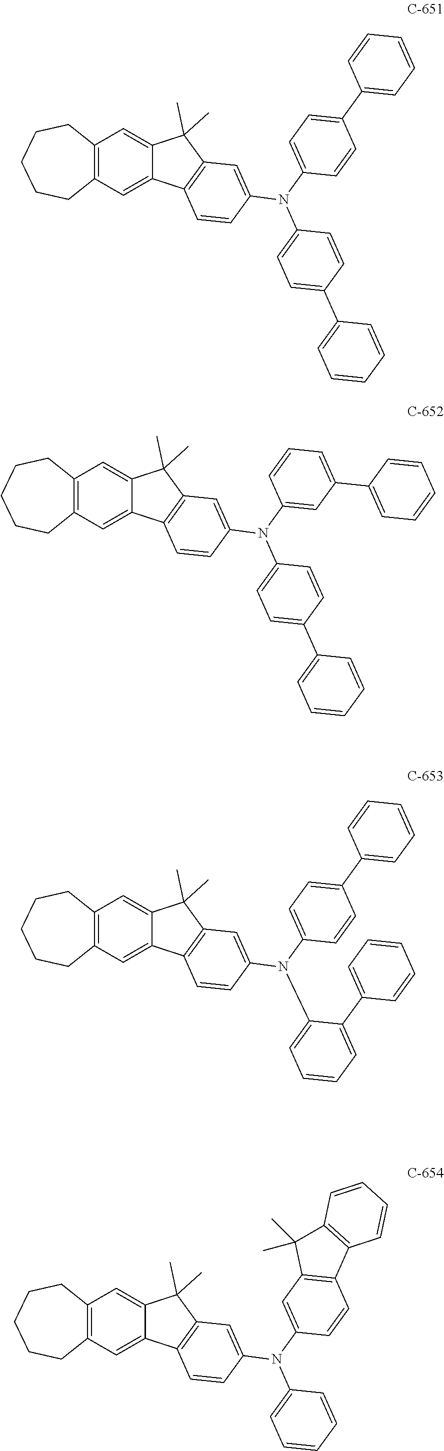 [SMILES: CC1(C)c2cc(N(c3ccc(-c4ccccc4)cc3)c3ccc(-c4ccccc4)cc3)ccc2-c2cc3c(cc21)CCCCC3 ; CC1(C)c2cc(N(c3ccc(-c4ccccc4)cc3)c3cccc(-c4ccccc4)c3)ccc2-c2cc3c(cc21)CCCCC3 ; CC1(C)c2cc(N(c3ccc(-c4ccccc4)cc3)c3ccccc3-c3ccccc3)ccc2-c2cc3c(cc21)CCCCC3 ; CC1(C)c2ccccc2-c2ccc(N(c3ccccc3)c3ccc4c(c3)C(C)(C)c3cc5c(cc3-4)CCCCC5)cc21]

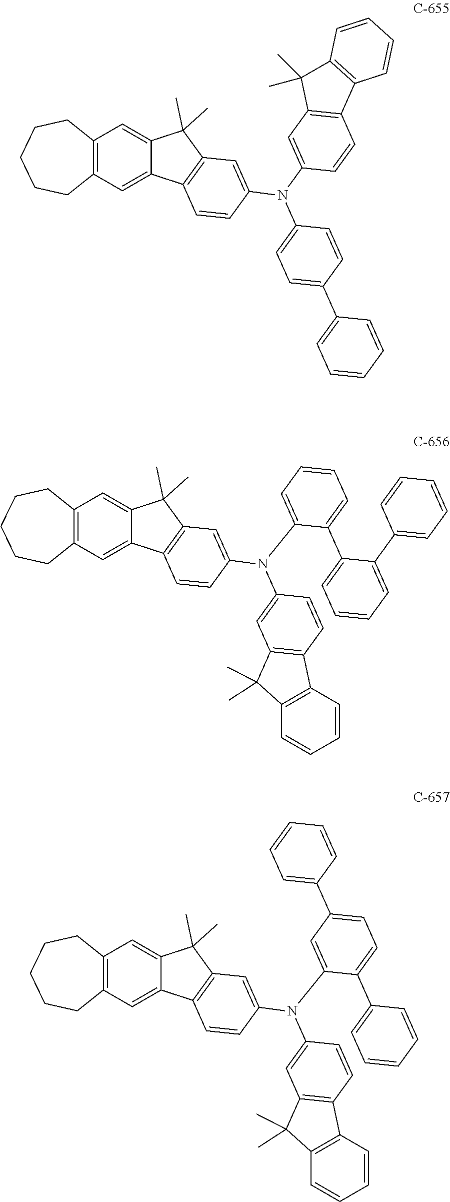 [SMILES: CC1(C)c2ccccc2-c2ccc(N(c3ccc(-c4ccccc4)cc3)c3ccc4c(c3)C(C)(C)c3cc5c(cc3-4)CCCCC5)cc21 ; CC1(C)c2ccccc2-c2ccc(N(c3ccc4c(c3)C(C)(C)c3cc5c(cc3-4)CCCCC5)c3ccccc3-c3ccccc3-c3ccccc3)cc21 ; CC1(C)c2ccccc2-c2ccc(N(c3ccc4c(c3)C(C)(C)c3cc5c(cc3-4)CCCCC5)c3cc(-c4ccccc4)ccc3-c3ccccc3)cc21]

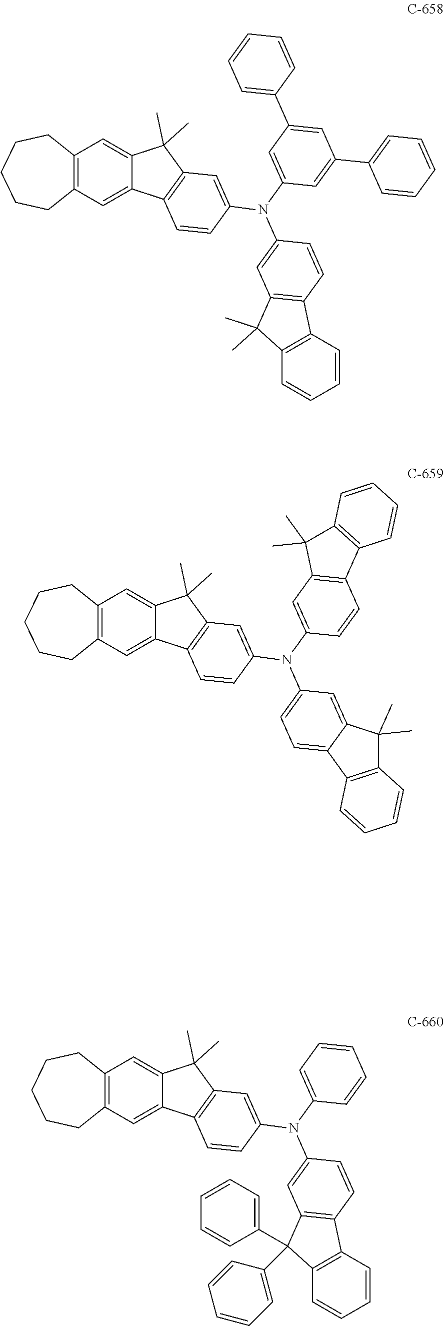 [SMILES: CC1(C)c2ccccc2-c2ccc(N(c3cc(-c4ccccc4)cc(-c4ccccc4)c3)c3ccc4c(c3)C(C)(C)c3cc5c(cc3-4)CCCCC5)cc21 ; CC1(C)c2ccccc2-c2ccc(N(c3ccc4c(c3)C(C)(C)c3ccccc3-4)c3ccc4c(c3)C(C)(C)c3cc5c(cc3-4)CCCCC5)cc21 ; CC1(C)c2cc(N(c3ccccc3)c3ccc4c(c3)C(c3ccccc3)(c3ccccc3)c3ccccc3-4)ccc2-c2cc3c(cc21)CCCCC3]

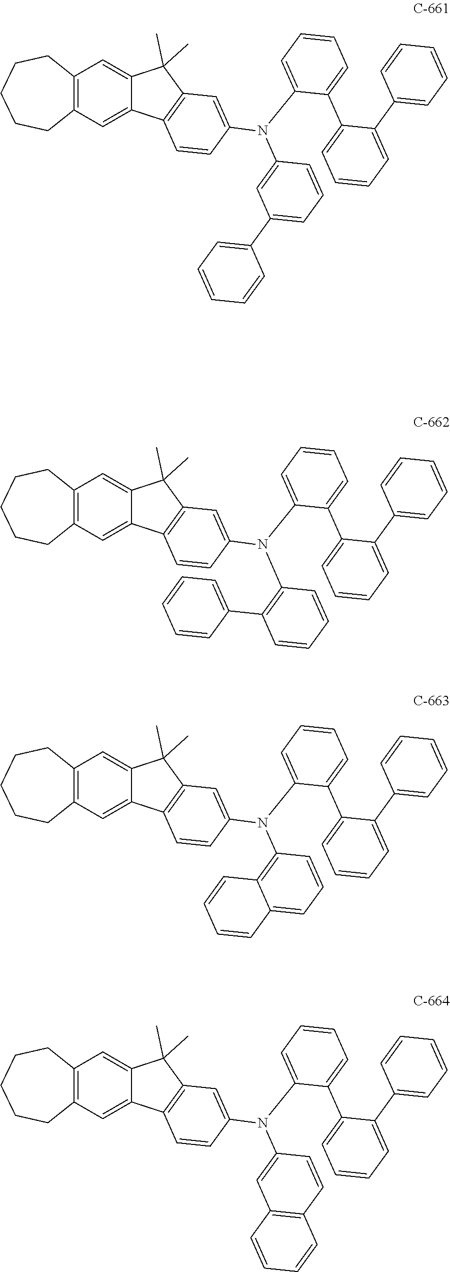 [SMILES: CC1(C)c2cc(N(c3cccc(-c4ccccc4)c3)c3ccccc3-c3ccccc3-c3ccccc3)ccc2-c2cc3c(cc21)CCCCC3 ; CC1(C)c2cc(N(c3ccccc3-c3ccccc3)c3ccccc3-c3ccccc3-c3ccccc3)ccc2-c2cc3c(cc21)CCCCC3 ; CC1(C)c2cc(N(c3ccccc3-c3ccccc3-c3ccccc3)c3cccc4ccccc34)ccc2-c2cc3c(cc21)CCCCC3 ; CC1(C)c2cc(N(c3ccc4ccccc4c3)c3ccccc3-c3ccccc3-c3ccccc3)ccc2-c2cc3c(cc21)CCCCC3]

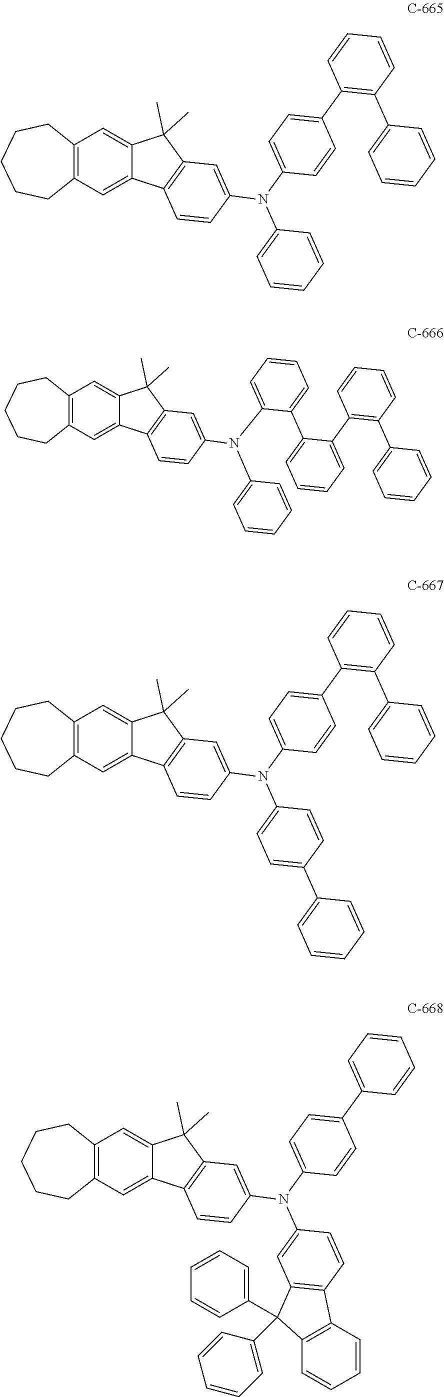 [SMILES: CC1(C)c2cc(N(c3ccccc3)c3ccc(-c4ccccc4-c4ccccc4)cc3)ccc2-c2cc3c(cc21)CCCCC3 ; CC1(C)c2cc(N(c3ccccc3)c3ccccc3-c3ccccc3-c3ccccc3-c3ccccc3)ccc2-c2cc3c(cc21)CCCCC3 ; CC1(C)c2cc(N(c3ccc(-c4ccccc4)cc3)c3ccc(-c4ccccc4-c4ccccc4)cc3)ccc2-c2cc3c(cc21)CCCCC3 ; CC1(C)c2cc(N(c3ccc(-c4ccccc4)cc3)c3ccc4c(c3)C(c3ccccc3)(c3ccccc3)c3ccccc3-4)ccc2-c2cc3c(cc21)CCCCC3]

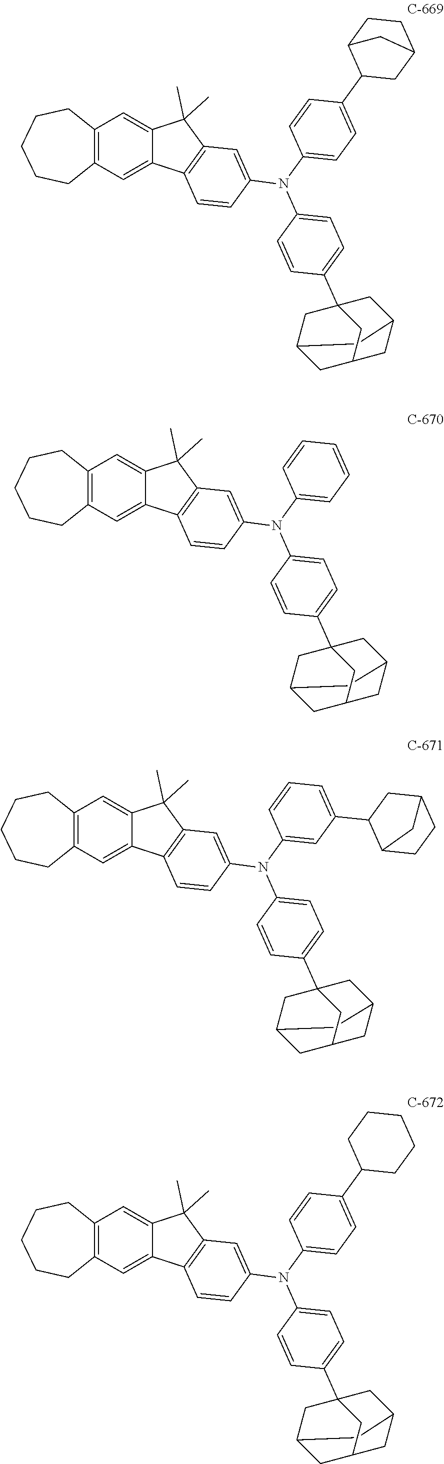 [SMILES: CC1(C)c2cc(N(c3ccc(C4CC5CCC4C5)cc3)c3ccc(C45CC6CC(CC(C6)C4)C5)cc3)ccc2-c2cc3c(cc21)CCCCC3 ; CC1(C)c2cc(N(c3ccccc3)c3ccc(C45CC6CC(CC(C6)C4)C5)cc3)ccc2-c2cc3c(cc21)CCCCC3 ; CC1(C)c2cc(N(c3ccc(C45CC6CC(CC(C6)C4)C5)cc3)c3cccc(C4CC5CCC4C5)c3)ccc2-c2cc3c(cc21)CCCCC3 ; CC1(C)c2cc(N(c3ccc(C4CCCCC4)cc3)c3ccc(C45CC6CC(CC(C6)C4)C5)cc3)ccc2-c2cc3c(cc21)CCCCC3]

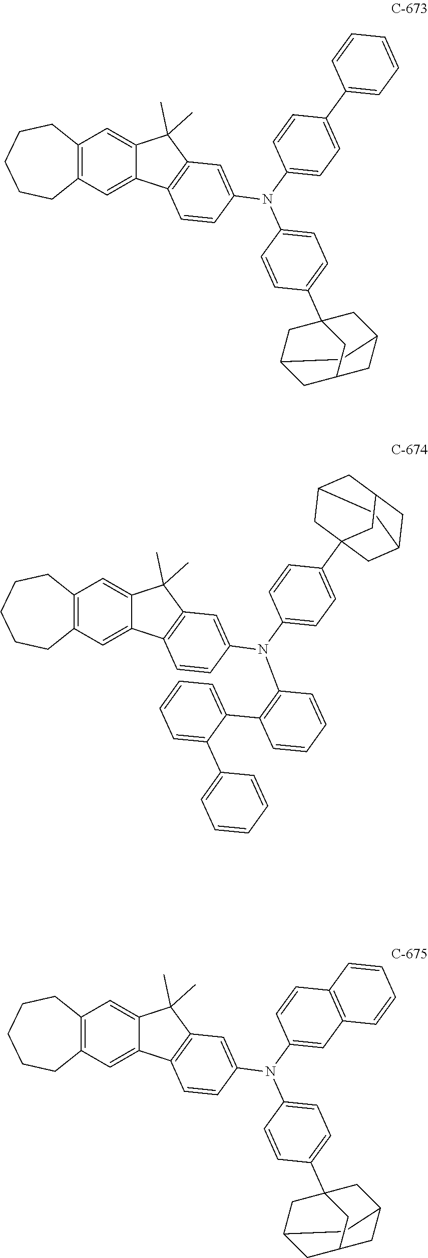 [SMILES: CC1(C)c2cc(N(c3ccc(-c4ccccc4)cc3)c3ccc(C45CC6CC(CC(C6)C4)C5)cc3)ccc2-c2cc3c(cc21)CCCCC3 ; CC1(C)c2cc(N(c3ccc(C45CC6CC(CC(C6)C4)C5)cc3)c3ccccc3-c3ccccc3-c3ccccc3)ccc2-c2cc3c(cc21)CCCCC3 ; CC1(C)c2cc(N(c3ccc(C45CC6CC(CC(C6)C4)C5)cc3)c3ccc4ccccc4c3)ccc2-c2cc3c(cc21)CCCCC3]

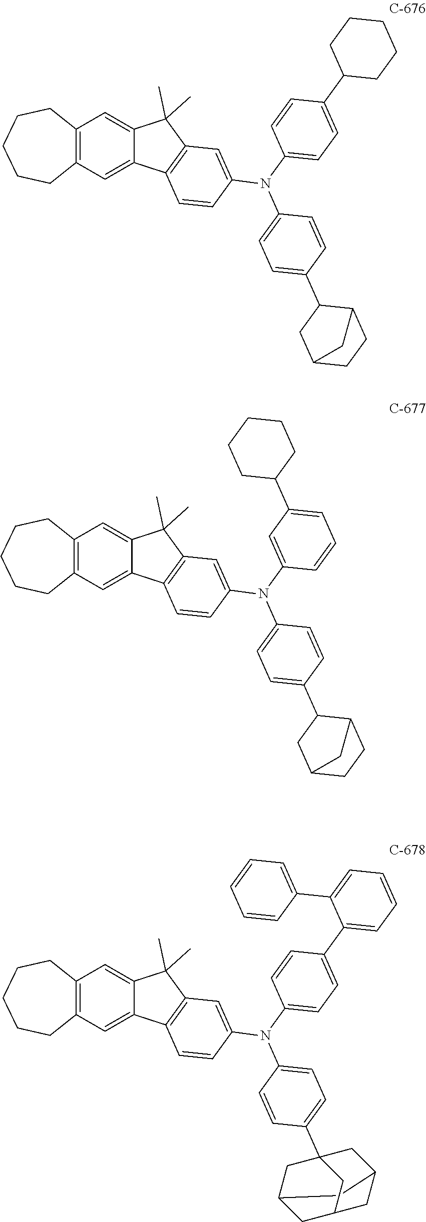 [SMILES: CC1(C)c2cc(N(c3ccc(C4CCCCC4)cc3)c3ccc(C4CC5CCC4C5)cc3)ccc2-c2cc3c(cc21)CCCCC3 ; CC1(C)c2cc(N(c3ccc(C4CC5CCC4C5)cc3)c3cccc(C4CCCCC4)c3)ccc2-c2cc3c(cc21)CCCCC3 ; CC1(C)c2cc(N(c3ccc(-c4ccccc4-c4ccccc4)cc3)c3ccc(C45CC6CC(CC(C6)C4)C5)cc3)ccc2-c2cc3c(cc21)CCCCC3]

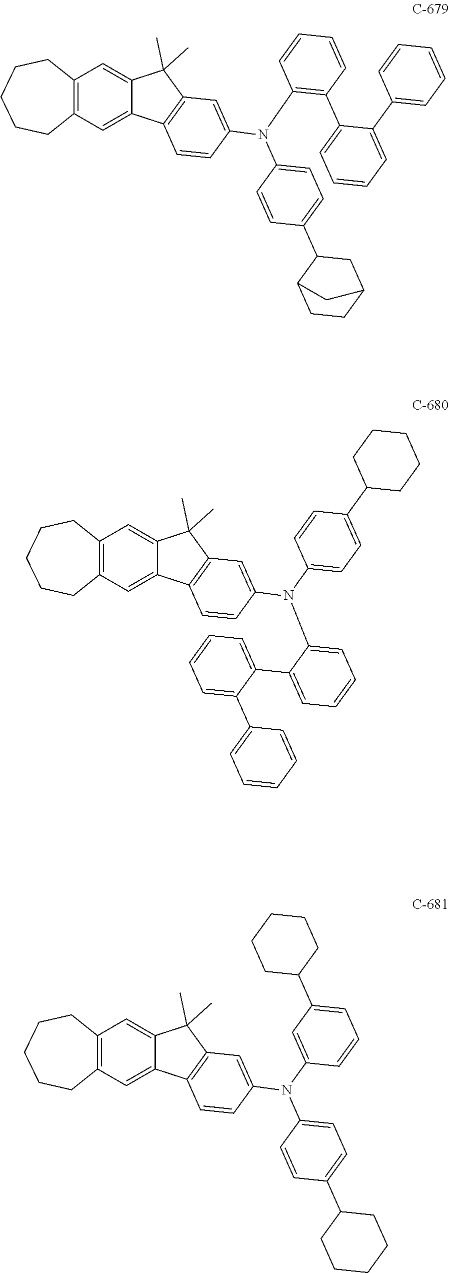 [SMILES: CC1(C)c2cc(N(c3ccc(C4CC5CCC4C5)cc3)c3ccccc3-c3ccccc3-c3ccccc3)ccc2-c2cc3c(cc21)CCCCC3 ; CC1(C)c2cc(N(c3ccc(C4CCCCC4)cc3)c3ccccc3-c3ccccc3-c3ccccc3)ccc2-c2cc3c(cc21)CCCCC3 ; CC1(C)c2cc(N(c3ccc(C4CCCCC4)cc3)c3cccc(C4CCCCC4)c3)ccc2-c2cc3c(cc21)CCCCC3]

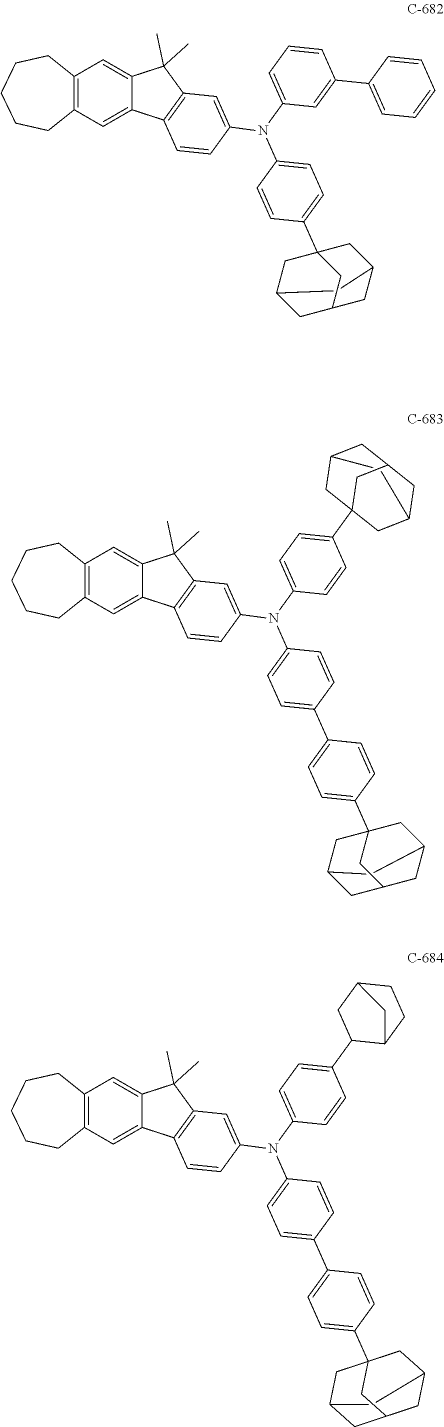 [SMILES: CC1(C)c2cc(N(c3ccc(C45CC6CC(CC(C6)C4)C5)cc3)c3cccc(-c4ccccc4)c3)ccc2-c2cc3c(cc21)CCCCC3 ; CC1(C)c2cc(N(c3ccc(-c4ccc(C56CC7CC(CC(C7)C5)C6)cc4)cc3)c3ccc(C45CC6CC(CC(C6)C4)C5)cc3)ccc2-c2cc3c(cc21)CCCCC3 ; CC1(C)c2cc(N(c3ccc(-c4ccc(C56CC7CC(CC(C7)C5)C6)cc4)cc3)c3ccc(C4CC5CCC4C5)cc3)ccc2-c2cc3c(cc21)CCCCC3]

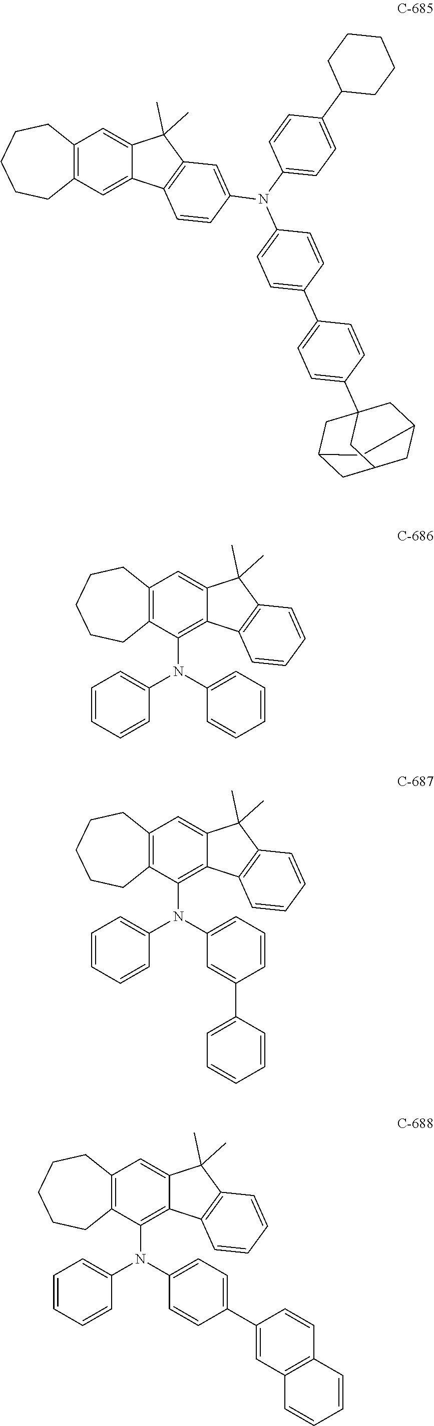 [SMILES: CC1(C)c2cc(N(c3ccc(-c4ccc(C56CC7CC(CC(C7)C5)C6)cc4)cc3)c3ccc(C4CCCCC4)cc3)ccc2-c2cc3c(cc21)CCCCC3 ; CC1(C)c2ccccc2-c2c1cc1c(c2N(c2ccccc2)c2ccccc2)CCCCC1 ; CC1(C)c2ccccc2-c2c1cc1c(c2N(c2ccccc2)c2cccc(-c3ccccc3)c2)CCCCC1 ; CC1(C)c2ccccc2-c2c1cc1c(c2N(c2ccccc2)c2ccc(-c3ccc4ccccc4c3)cc2)CCCCC1]

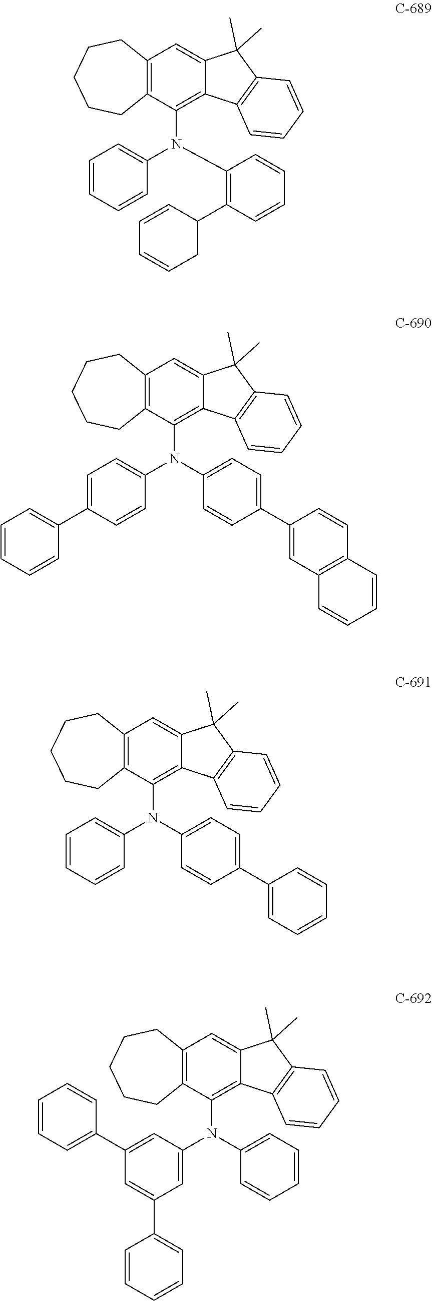 [SMILES: CC1(C)c2ccccc2-c2c1cc1c(c2N(c2ccccc2)c2ccccc2C2C=CC=CC2)CCCCC1 ; CC1(C)c2ccccc2-c2c1cc1c(c2N(c2ccc(-c3ccccc3)cc2)c2ccc(-c3ccc4ccccc4c3)cc2)CCCCC1 ; CC1(C)c2ccccc2-c2c1cc1c(c2N(c2ccccc2)c2ccc(-c3ccccc3)cc2)CCCCC1 ; CC1(C)c2ccccc2-c2c1cc1c(c2N(c2ccccc2)c2cc(-c3ccccc3)cc(-c3ccccc3)c2)CCCCC1]

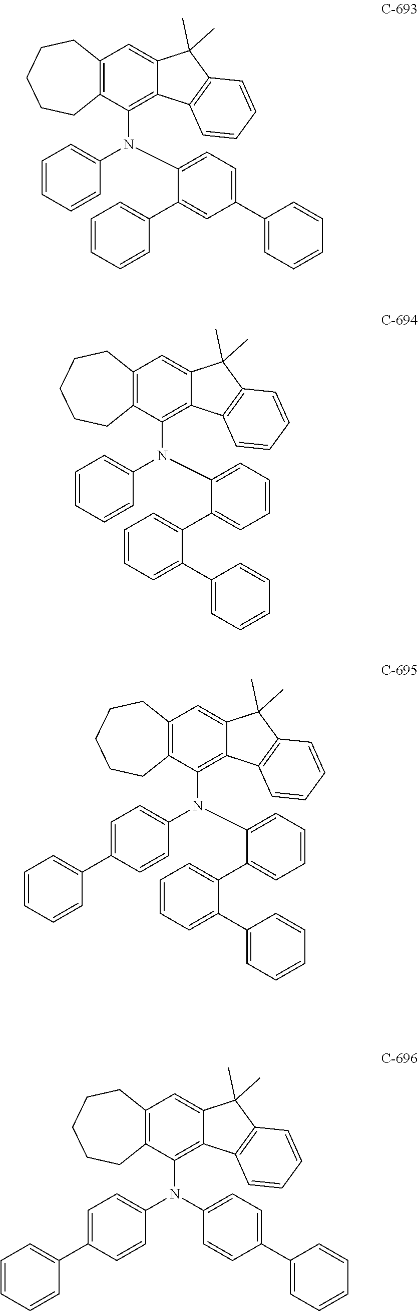 [SMILES: CC1(C)c2ccccc2-c2c1cc1c(c2N(c2ccccc2)c2ccc(-c3ccccc3)cc2-c2ccccc2)CCCCC1 ; CC1(C)c2ccccc2-c2c1cc1c(c2N(c2ccccc2)c2ccccc2-c2ccccc2-c2ccccc2)CCCCC1 ; CC1(C)c2ccccc2-c2c1cc1c(c2N(c2ccc(-c3ccccc3)cc2)c2ccccc2-c2ccccc2-c2ccccc2)CCCCC1 ; CC1(C)c2ccccc2-c2c1cc1c(c2N(c2ccc(-c3ccccc3)cc2)c2ccc(-c3ccccc3)cc2)CCCCC1]

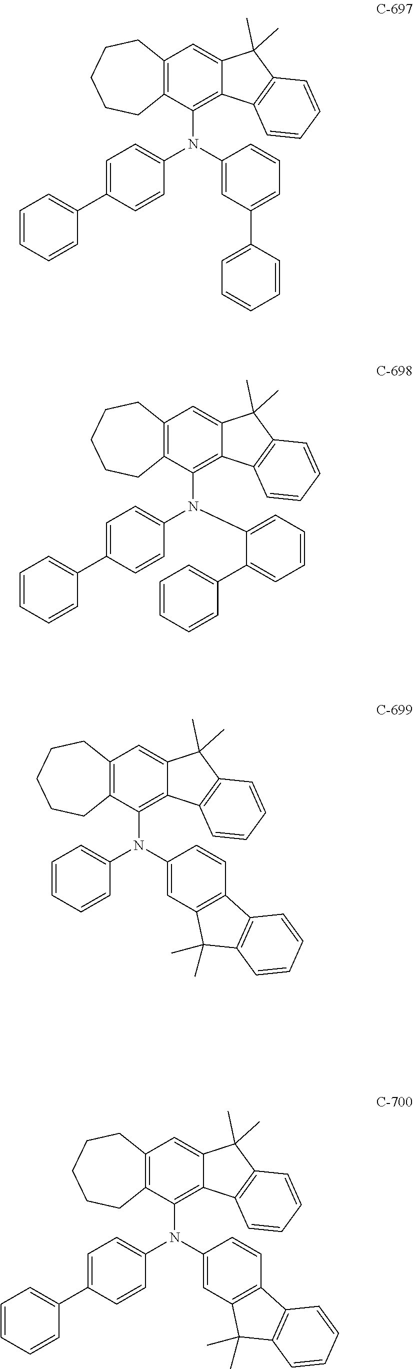 [SMILES: CC1(C)c2ccccc2-c2c1cc1c(c2N(c2ccc(-c3ccccc3)cc2)c2cccc(-c3ccccc3)c2)CCCCC1 ; CC1(C)c2ccccc2-c2c1cc1c(c2N(c2ccc(-c3ccccc3)cc2)c2ccccc2-c2ccccc2)CCCCC1 ; CC1(C)c2ccccc2-c2ccc(N(c3ccccc3)c3c4c(cc5c3-c3ccccc3C5(C)C)CCCCC4)cc21 ; CC1(C)c2ccccc2-c2ccc(N(c3ccc(-c4ccccc4)cc3)c3c4c(cc5c3-c3ccccc3C5(C)C)CCCCC4)cc21]

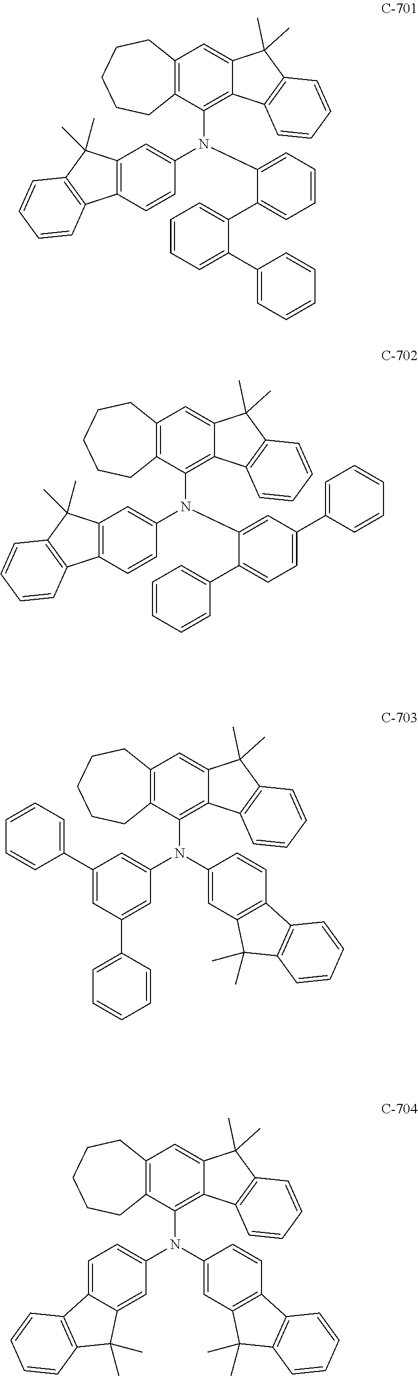 [SMILES: CC1(C)c2ccccc2-c2ccc(N(c3ccccc3-c3ccccc3-c3ccccc3)c3c4c(cc5c3-c3ccccc3C5(C)C)CCCCC4)cc21 ; CC1(C)c2ccccc2-c2ccc(N(c3cc(-c4ccccc4)ccc3-c3ccccc3)c3c4c(cc5c3-c3ccccc3C5(C)C)CCCCC4)cc21 ; CC1(C)c2ccccc2-c2ccc(N(c3cc(-c4ccccc4)cc(-c4ccccc4)c3)c3c4c(cc5c3-c3ccccc3C5(C)C)CCCCC4)cc21 ; CC1(C)c2ccccc2-c2ccc(N(c3ccc4c(c3)C(C)(C)c3ccccc3-4)c3c4c(cc5c3-c3ccccc3C5(C)C)CCCCC4)cc21]

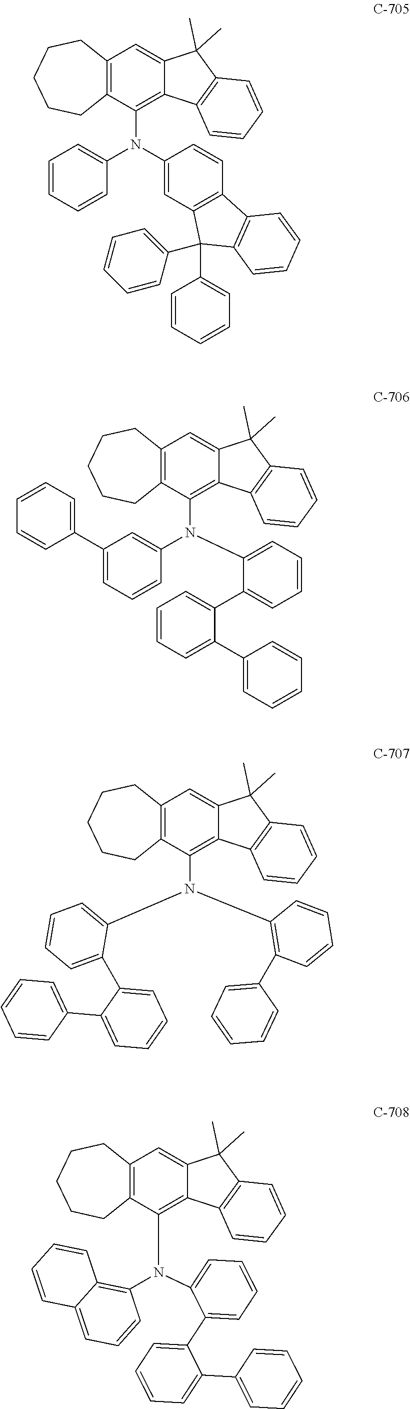 [SMILES: CC1(C)c2ccccc2-c2c1cc1c(c2N(c2ccccc2)c2ccc3c(c2)C(c2ccccc2)(c2ccccc2)c2ccccc2-3)CCCCC1 ; CC1(C)c2ccccc2-c2c1cc1c(c2N(c2cccc(-c3ccccc3)c2)c2ccccc2-c2ccccc2-c2ccccc2)CCCCC1 ; CC1(C)c2ccccc2-c2c1cc1c(c2N(c2ccccc2-c2ccccc2)c2ccccc2-c2ccccc2-c2ccccc2)CCCCC1 ; CC1(C)c2ccccc2-c2c1cc1c(c2N(c2ccccc2-c2ccccc2-c2ccccc2)c2cccc3ccccc23)CCCCC1]

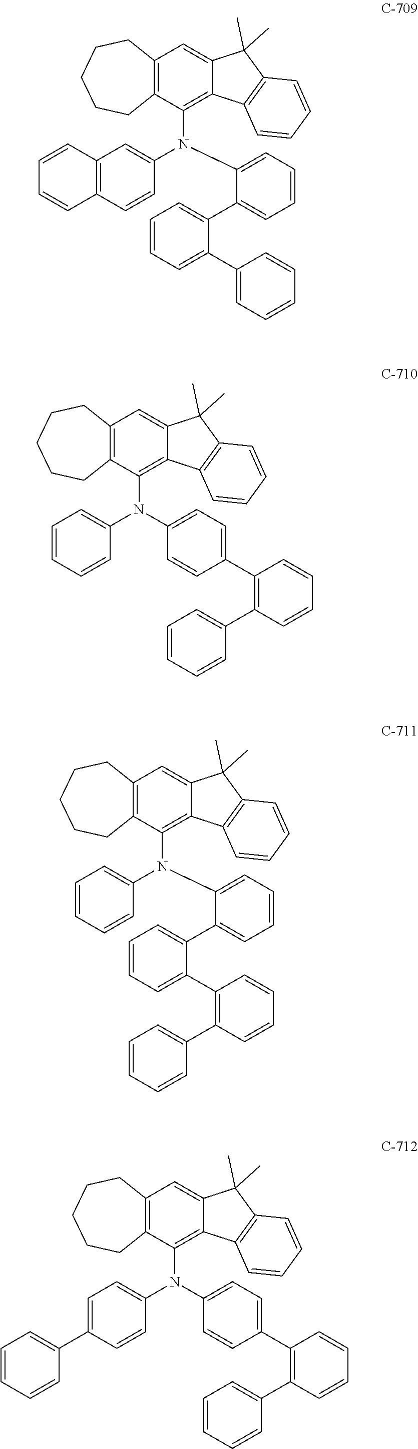 [SMILES: CC1(C)c2ccccc2-c2c1cc1c(c2N(c2ccc3ccccc3c2)c2ccccc2-c2ccccc2-c2ccccc2)CCCCC1 ; CC1(C)c2ccccc2-c2c1cc1c(c2N(c2ccccc2)c2ccc(-c3ccccc3-c3ccccc3)cc2)CCCCC1 ; CC1(C)c2ccccc2-c2c1cc1c(c2N(c2ccccc2)c2ccccc2-c2ccccc2-c2ccccc2-c2ccccc2)CCCCC1 ; CC1(C)c2ccccc2-c2c1cc1c(c2N(c2ccc(-c3ccccc3)cc2)c2ccc(-c3ccccc3-c3ccccc3)cc2)CCCCC1]

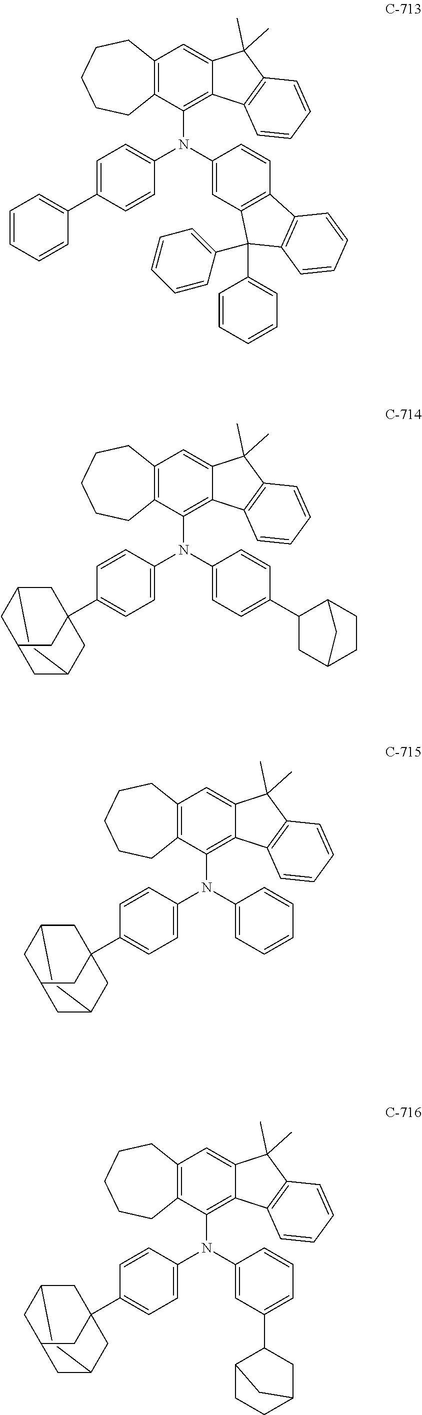 [SMILES: CC1(C)c2ccccc2-c2c1cc1c(c2N(c2ccc(-c3ccccc3)cc2)c2ccc3c(c2)C(c2ccccc2)(c2ccccc2)c2ccccc2-3)CCCCC1 ; CC1(C)c2ccccc2-c2c1cc1c(c2N(c2ccc(C3CC4CCC3C4)cc2)c2ccc(C34CC5CC(CC(C5)C3)C4)cc2)CCCCC1 ; CC1(C)c2ccccc2-c2c1cc1c(c2N(c2ccccc2)c2ccc(C34CC5CC(CC(C5)C3)C4)cc2)CCCCC1 ; CC1(C)c2ccccc2-c2c1cc1c(c2N(c2ccc(C34CC5CC(CC(C5)C3)C4)cc2)c2cccc(C3CC4CCC3C4)c2)CCCCC1]

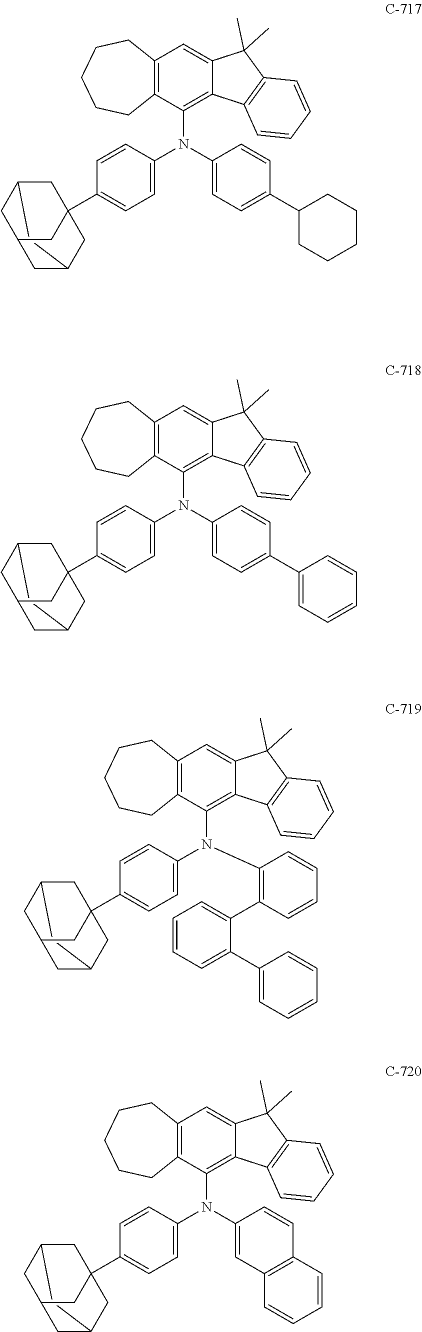 [SMILES: CC1(C)c2ccccc2-c2c1cc1c(c2N(c2ccc(C3CCCCC3)cc2)c2ccc(C34CC5CC(CC(C5)C3)C4)cc2)CCCCC1 ; CC1(C)c2ccccc2-c2c1cc1c(c2N(c2ccc(-c3ccccc3)cc2)c2ccc(C34CC5CC(CC(C5)C3)C4)cc2)CCCCC1 ; CC1(C)c2ccccc2-c2c1cc1c(c2N(c2ccc(C34CC5CC(CC(C5)C3)C4)cc2)c2ccccc2-c2ccccc2-c2ccccc2)CCCCC1 ; CC1(C)c2ccccc2-c2c1cc1c(c2N(c2ccc(C34CC5CC(CC(C5)C3)C4)cc2)c2ccc3ccccc3c2)CCCCC1]

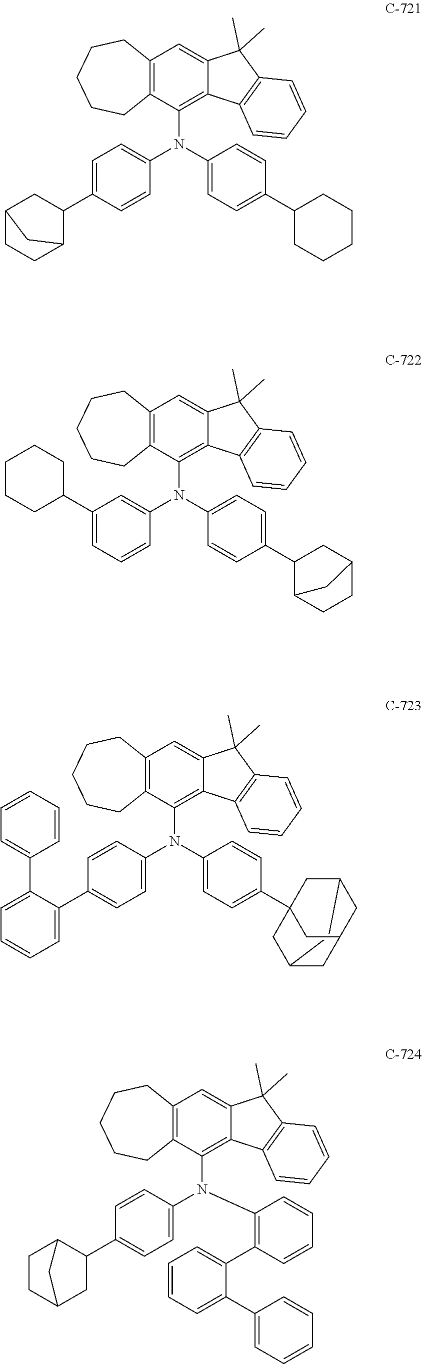 [SMILES: CC1(C)c2ccccc2-c2c1cc1c(c2N(c2ccc(C3CCCCC3)cc2)c2ccc(C3CC4CCC3C4)cc2)CCCCC1 ; CC1(C)c2ccccc2-c2c1cc1c(c2N(c2ccc(C3CC4CCC3C4)cc2)c2cccc(C3CCCCC3)c2)CCCCC1 ; CC1(C)c2ccccc2-c2c1cc1c(c2N(c2ccc(-c3ccccc3-c3ccccc3)cc2)c2ccc(C34CC5CC(CC(C5)C3)C4)cc2)CCCCC1 ; CC1(C)c2ccccc2-c2c1cc1c(c2N(c2ccc(C3CC4CCC3C4)cc2)c2ccccc2-c2ccccc2-c2ccccc2)CCCCC1]

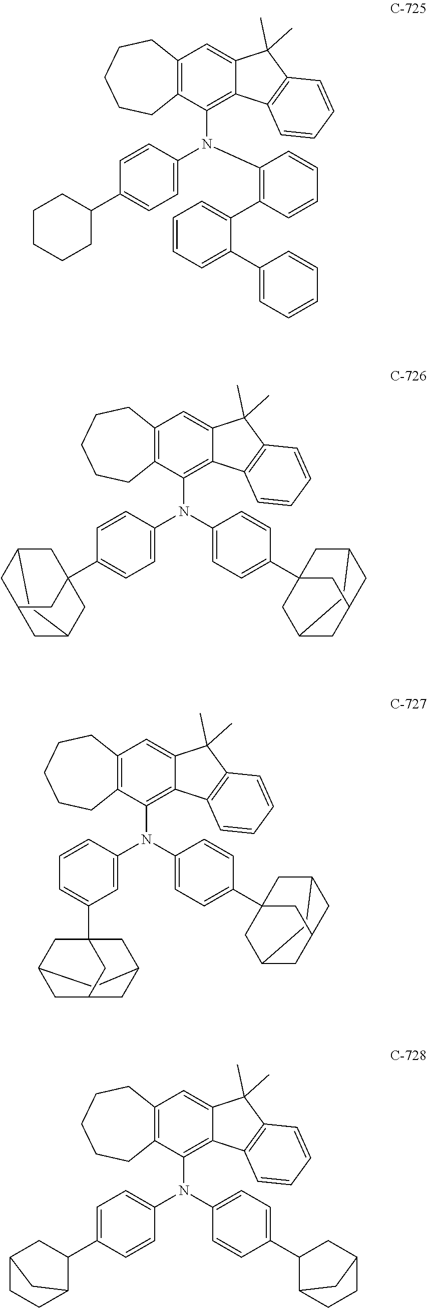 [SMILES: CC1(C)c2ccccc2-c2c1cc1c(c2N(c2ccc(C3CCCCC3)cc2)c2ccccc2-c2ccccc2-c2ccccc2)CCCCC1 ; CC1(C)c2ccccc2-c2c1cc1c(c2N(c2ccc(C34CC5CC(CC(C5)C3)C4)cc2)c2ccc(C34CC5CC(CC(C5)C3)C4)cc2)CCCCC1 ; CC1(C)c2ccccc2-c2c1cc1c(c2N(c2ccc(C34CC5CC(CC(C5)C3)C4)cc2)c2cccc(C34CC5CC(CC(C5)C3)C4)c2)CCCCC1 ; CC1(C)c2ccccc2-c2c1cc1c(c2N(c2ccc(C3CC4CCC3C4)cc2)c2ccc(C3CC4CCC3C4)cc2)CCCCC1]

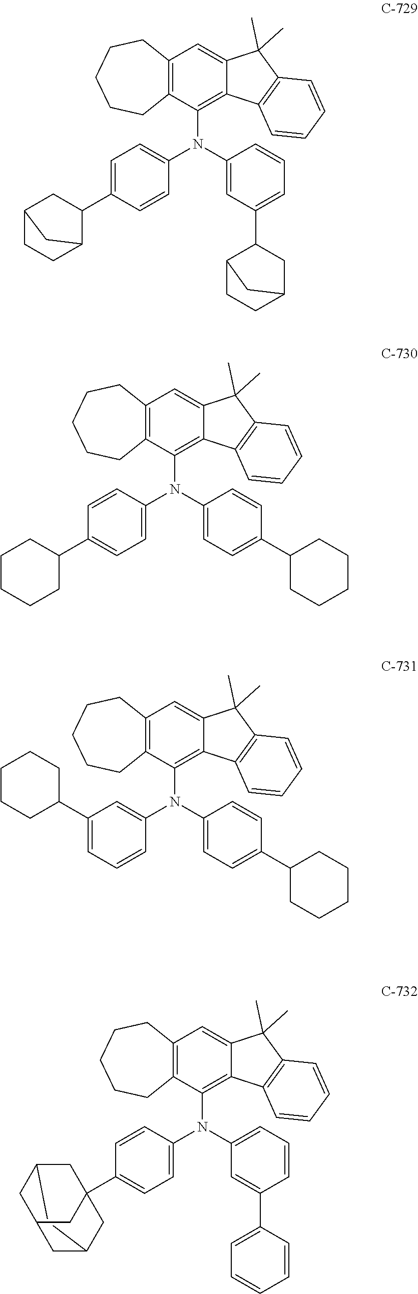 [SMILES: CC1(C)c2ccccc2-c2c1cc1c(c2N(c2ccc(C3CC4CCC3C4)cc2)c2cccc(C3CC4CCC3C4)c2)CCCCC1 ; CC1(C)c2ccccc2-c2c1cc1c(c2N(c2ccc(C3CCCCC3)cc2)c2ccc(C3CCCCC3)cc2)CCCCC1 ; CC1(C)c2ccccc2-c2c1cc1c(c2N(c2ccc(C3CCCCC3)cc2)c2cccc(C3CCCCC3)c2)CCCCC1 ; CC1(C)c2ccccc2-c2c1cc1c(c2N(c2ccc(C34CC5CC(CC(C5)C3)C4)cc2)c2cccc(-c3ccccc3)c2)CCCCC1]

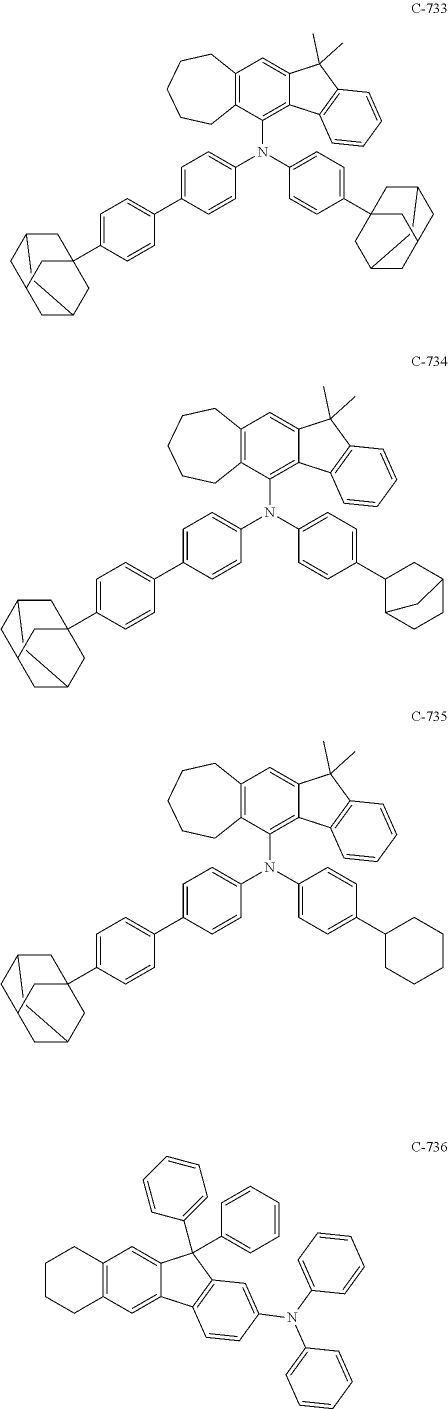 [SMILES: CC1(C)c2ccccc2-c2c1cc1c(c2N(c2ccc(-c3ccc(C45CC6CC(CC(C6)C4)C5)cc3)cc2)c2ccc(C34CC5CC(CC(C5)C3)C4)cc2)CCCCC1 ; CC1(C)c2ccccc2-c2c1cc1c(c2N(c2ccc(-c3ccc(C45CC6CC(CC(C6)C4)C5)cc3)cc2)c2ccc(C3CC4CCC3C4)cc2)CCCCC1 ; CC1(C)c2ccccc2-c2c1cc1c(c2N(c2ccc(-c3ccc(C45CC6CC(CC(C6)C4)C5)cc3)cc2)c2ccc(C3CCCCC3)cc2)CCCCC1 ; c1ccc(N(c2ccccc2)c2ccc3c(c2)C(c2ccccc2)(c2ccccc2)c2cc4c(cc2-3)CCCC4)cc1]

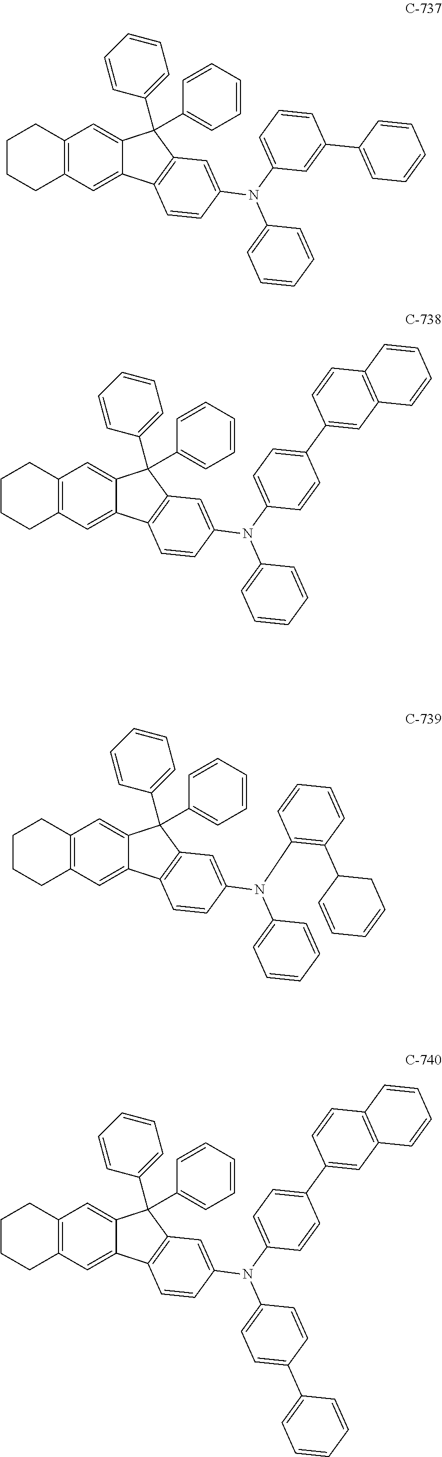 [SMILES: c1ccc(-c2cccc(N(c3ccccc3)c3ccc4c(c3)C(c3ccccc3)(c3ccccc3)c3cc5c(cc3-4)CCCC5)c2)cc1 ; c1ccc(N(c2ccc(-c3ccc4ccccc4c3)cc2)c2ccc3c(c2)C(c2ccccc2)(c2ccccc2)c2cc4c(cc2-3)CCCC4)cc1 ; C1=CCC(c2ccccc2N(c2ccccc2)c2ccc3c(c2)C(c2ccccc2)(c2ccccc2)c2cc4c(cc2-3)CCCC4)C=C1 ; c1ccc(-c2ccc(N(c3ccc(-c4ccc5ccccc5c4)cc3)c3ccc4c(c3)C(c3ccccc3)(c3ccccc3)c3cc5c(cc3-4)CCCC5)cc2)cc1]

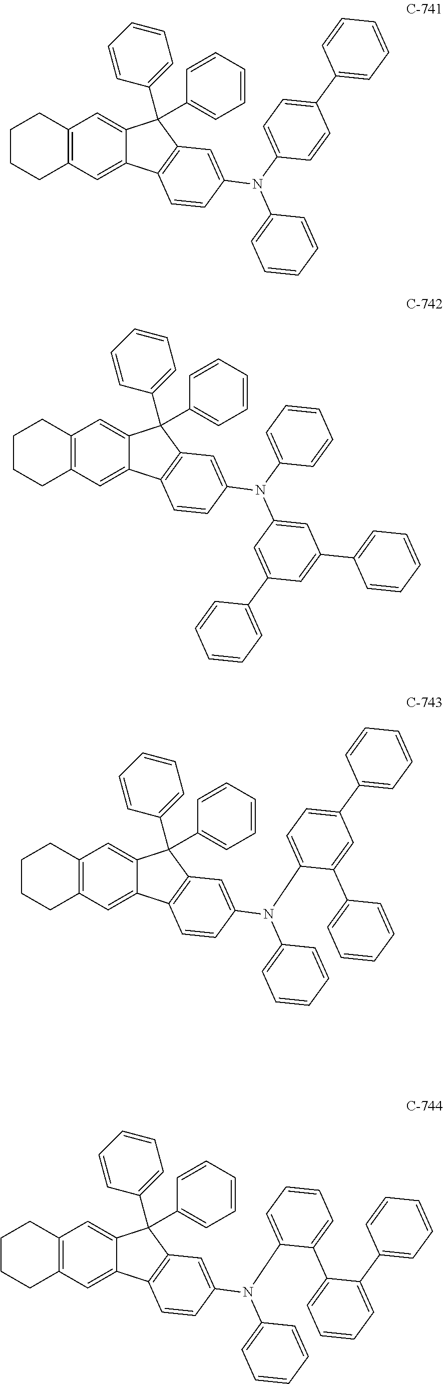 [SMILES: c1ccc(-c2ccc(N(c3ccccc3)c3ccc4c(c3)C(c3ccccc3)(c3ccccc3)c3cc5c(cc3-4)CCCC5)cc2)cc1 ; c1ccc(-c2cc(-c3ccccc3)cc(N(c3ccccc3)c3ccc4c(c3)C(c3ccccc3)(c3ccccc3)c3cc5c(cc3-4)CCCC5)c2)cc1 ; c1ccc(-c2ccc(N(c3ccccc3)c3ccc4c(c3)C(c3ccccc3)(c3ccccc3)c3cc5c(cc3-4)CCCC5)c(-c3ccccc3)c2)cc1 ; c1ccc(-c2ccccc2-c2ccccc2N(c2ccccc2)c2ccc3c(c2)C(c2ccccc2)(c2ccccc2)c2cc4c(cc2-3)CCCC4)cc1]

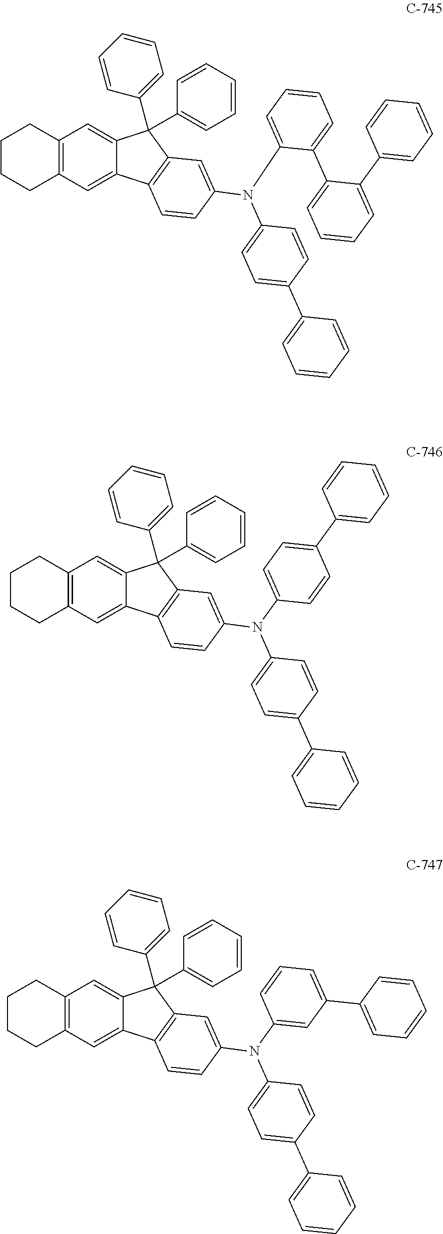 [SMILES: c1ccc(-c2ccc(N(c3ccc4c(c3)C(c3ccccc3)(c3ccccc3)c3cc5c(cc3-4)CCCC5)c3ccccc3-c3ccccc3-c3ccccc3)cc2)cc1 ; c1ccc(-c2ccc(N(c3ccc(-c4ccccc4)cc3)c3ccc4c(c3)C(c3ccccc3)(c3ccccc3)c3cc5c(cc3-4)CCCC5)cc2)cc1 ; c1ccc(-c2ccc(N(c3cccc(-c4ccccc4)c3)c3ccc4c(c3)C(c3ccccc3)(c3ccccc3)c3cc5c(cc3-4)CCCC5)cc2)cc1]

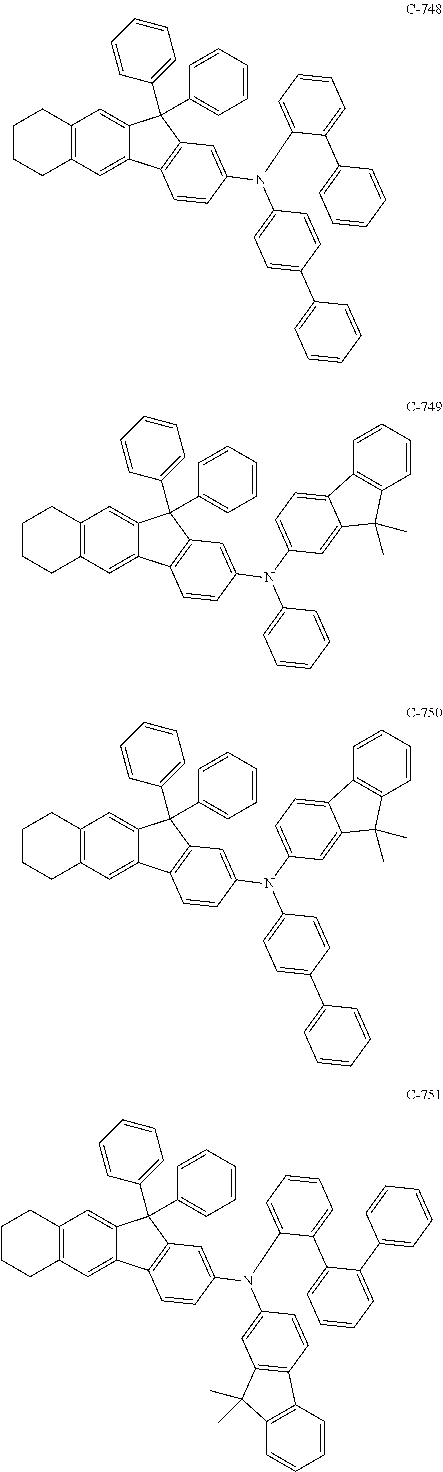 [SMILES: c1ccc(-c2ccc(N(c3ccc4c(c3)C(c3ccccc3)(c3ccccc3)c3cc5c(cc3-4)CCCC5)c3ccccc3-c3ccccc3)cc2)cc1 ; CC1(C)c2ccccc2-c2ccc(N(c3ccccc3)c3ccc4c(c3)C(c3ccccc3)(c3ccccc3)c3cc5c(cc3-4)CCCC5)cc21 ; CC1(C)c2ccccc2-c2ccc(N(c3ccc(-c4ccccc4)cc3)c3ccc4c(c3)C(c3ccccc3)(c3ccccc3)c3cc5c(cc3-4)CCCC5)cc21 ; CC1(C)c2ccccc2-c2ccc(N(c3ccc4c(c3)C(c3ccccc3)(c3ccccc3)c3cc5c(cc3-4)CCCC5)c3ccccc3-c3ccccc3-c3ccccc3)cc21]

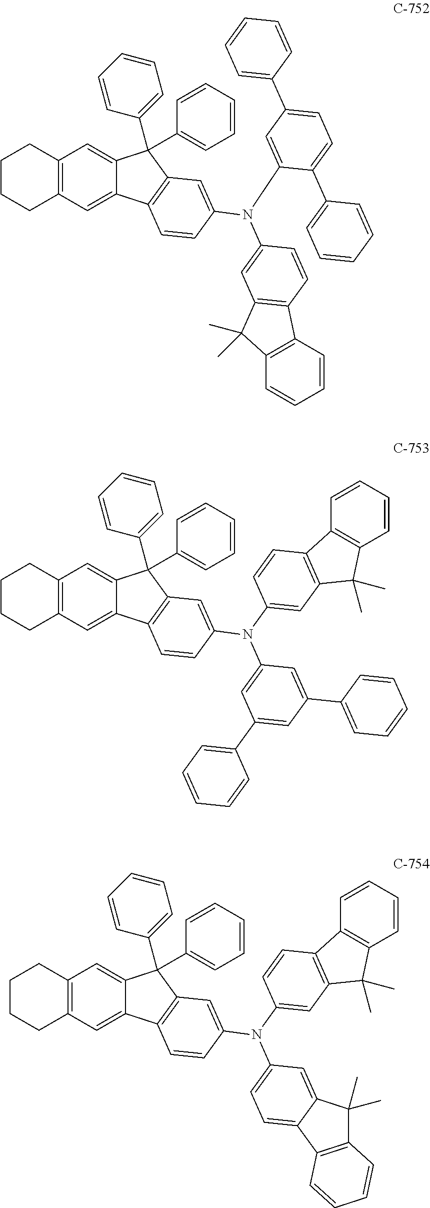 [SMILES: CC1(C)c2ccccc2-c2ccc(N(c3ccc4c(c3)C(c3ccccc3)(c3ccccc3)c3cc5c(cc3-4)CCCC5)c3cc(-c4ccccc4)ccc3-c3ccccc3)cc21 ; CC1(C)c2ccccc2-c2ccc(N(c3cc(-c4ccccc4)cc(-c4ccccc4)c3)c3ccc4c(c3)C(c3ccccc3)(c3ccccc3)c3cc5c(cc3-4)CCCC5)cc21 ; CC1(C)c2ccccc2-c2ccc(N(c3ccc4c(c3)C(C)(C)c3ccccc3-4)c3ccc4c(c3)C(c3ccccc3)(c3ccccc3)c3cc5c(cc3-4)CCCC5)cc21]

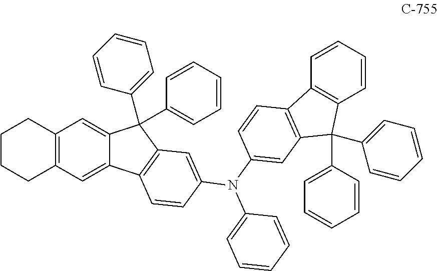 [SMILES: c1ccc(N(c2ccc3c(c2)C(c2ccccc2)(c2ccccc2)c2ccccc2-3)c2ccc3c(c2)C(c2ccccc2)(c2ccccc2)c2cc4c(cc2-3)CCCC4)cc1]

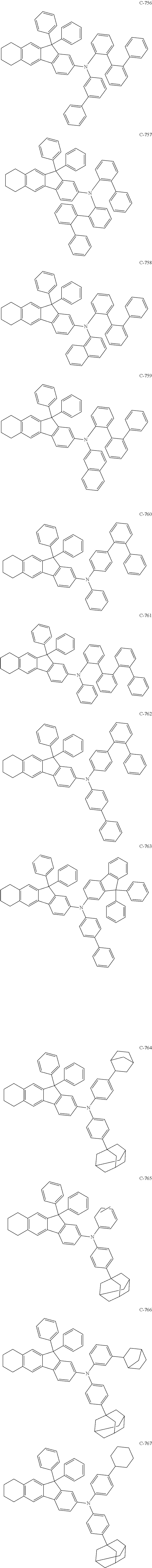

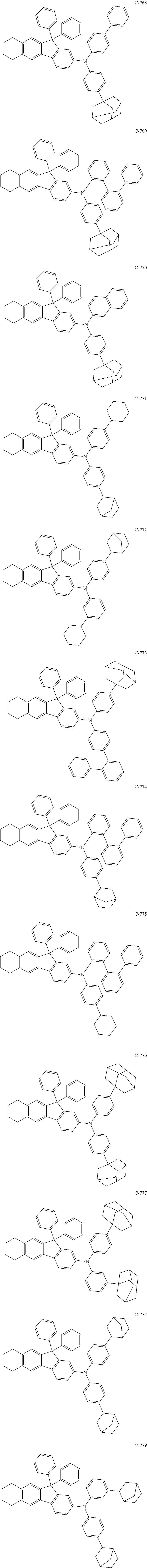

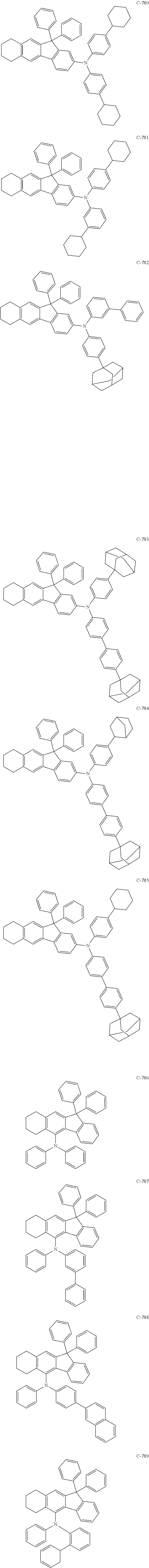

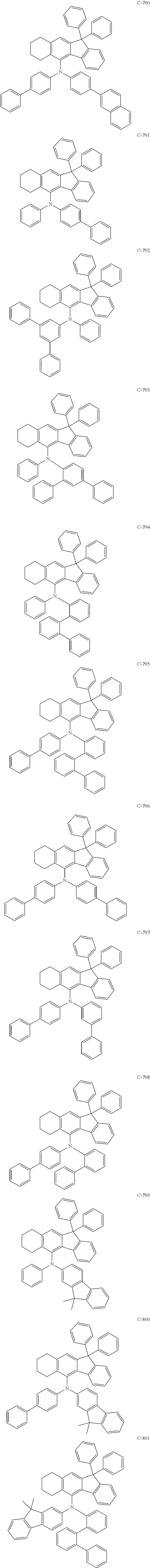

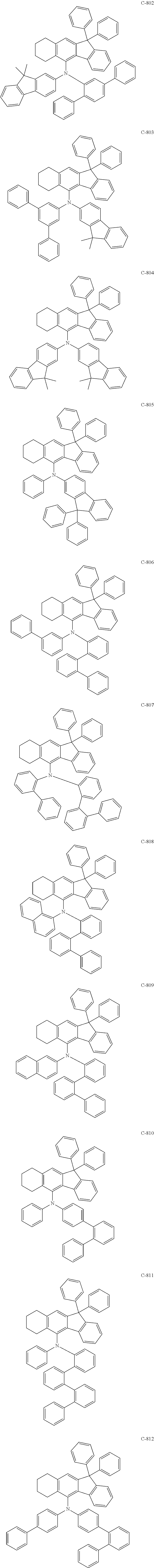

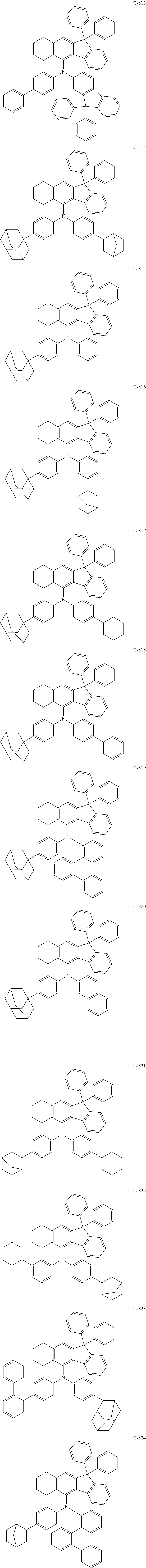

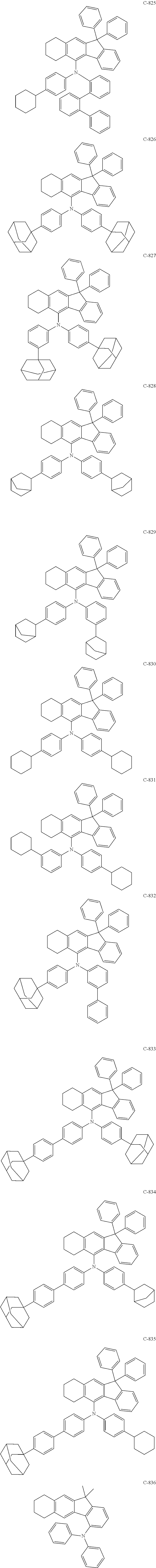

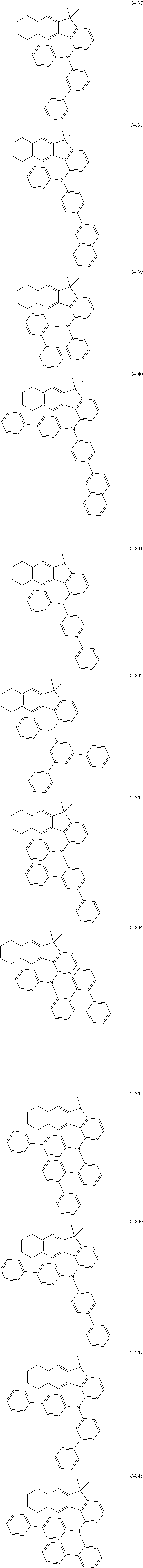

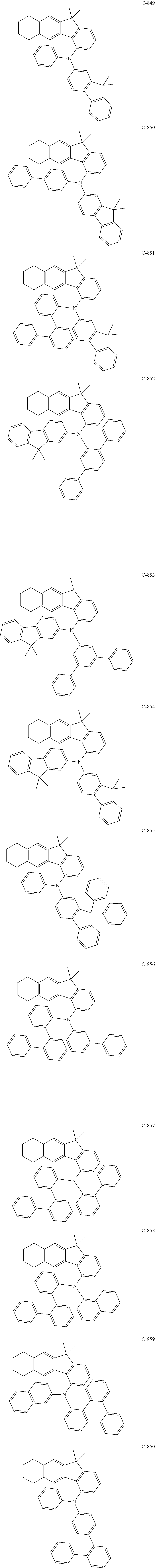

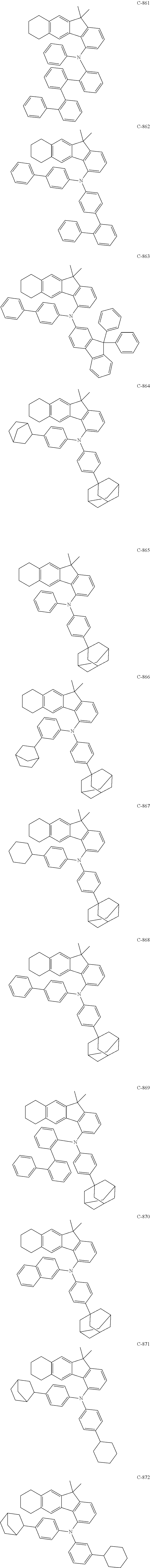

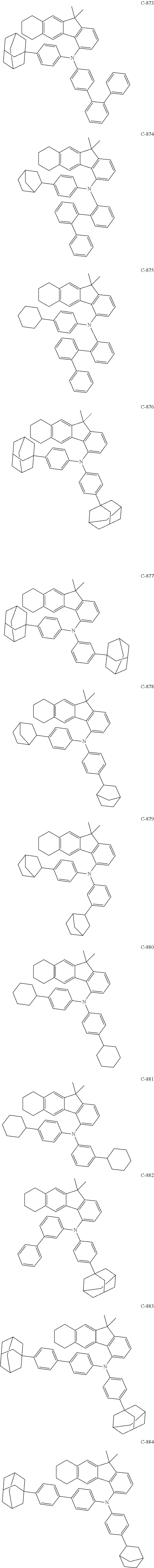

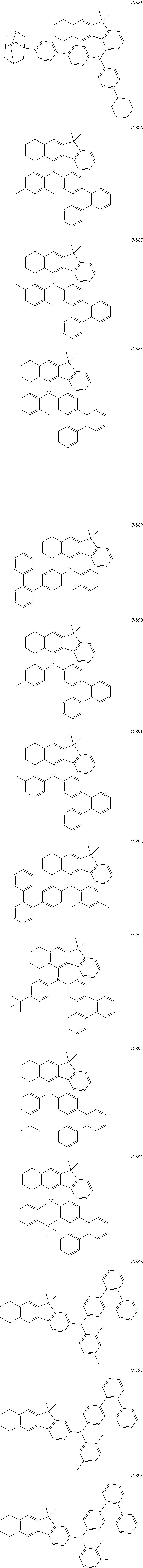

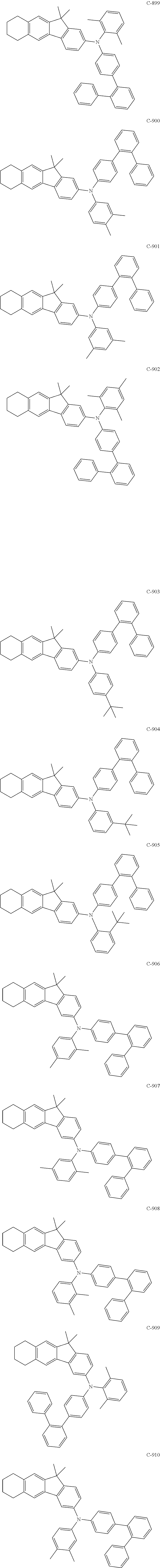

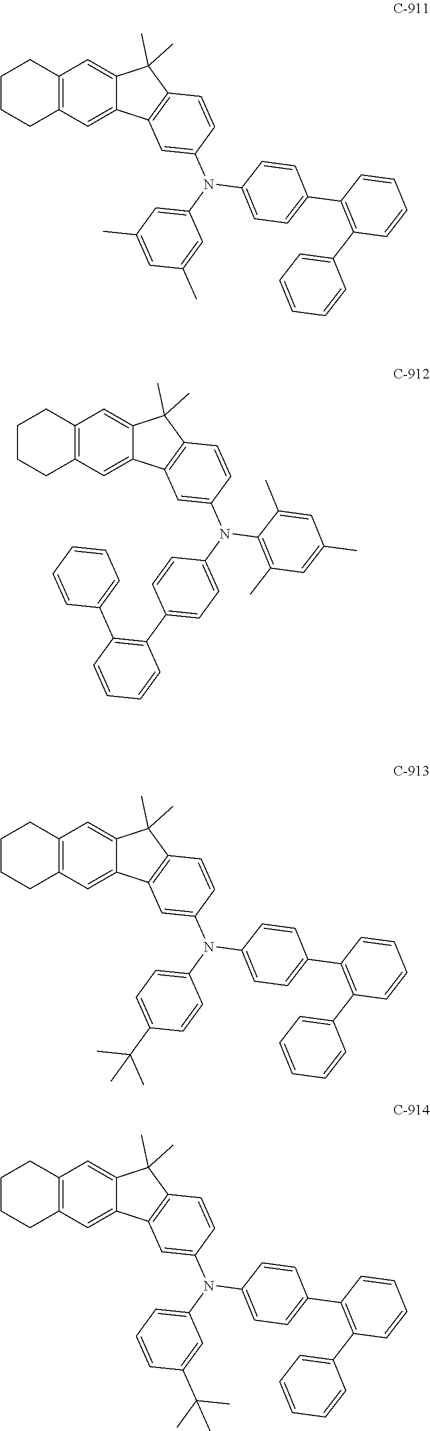 [SMILES: Cc1cc(C)cc(N(c2ccc(-c3ccccc3-c3ccccc3)cc2)c2ccc3c(c2)-c2cc4c(cc2C3(C)C)CCCC4)c1 ; Cc1cc(C)c(N(c2ccc(-c3ccccc3-c3ccccc3)cc2)c2ccc3c(c2)-c2cc4c(cc2C3(C)C)CCCC4)c(C)c1 ; CC(C)(C)c1ccc(N(c2ccc(-c3ccccc3-c3ccccc3)cc2)c2ccc3c(c2)-c2cc4c(cc2C3(C)C)CCCC4)cc1 ; CC(C)(C)c1cccc(N(c2ccc(-c3ccccc3-c3ccccc3)cc2)c2ccc3c(c2)-c2cc4c(cc2C3(C)C)CCCC4)c1]

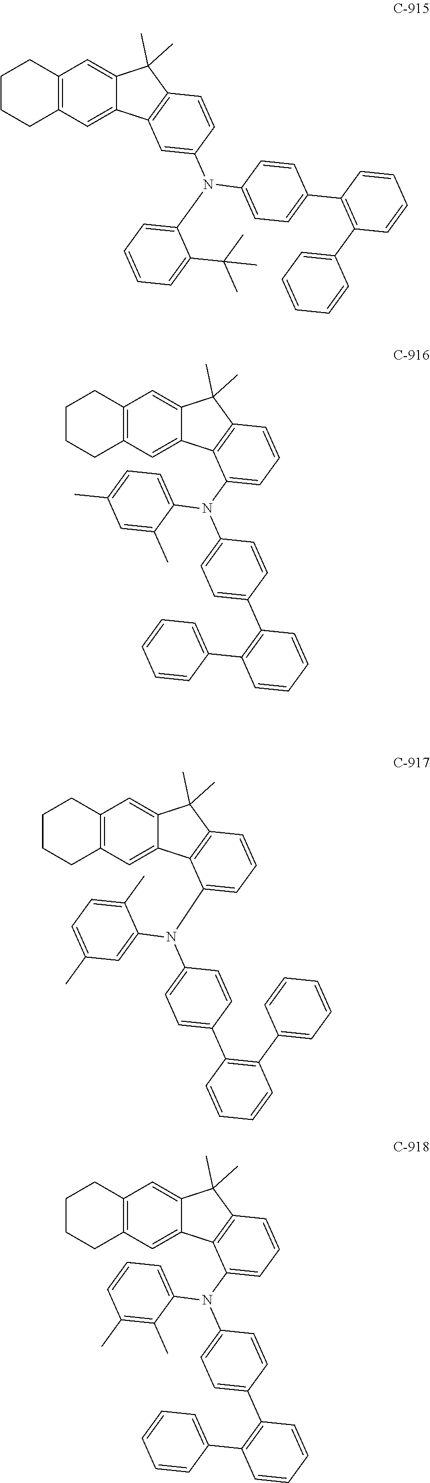

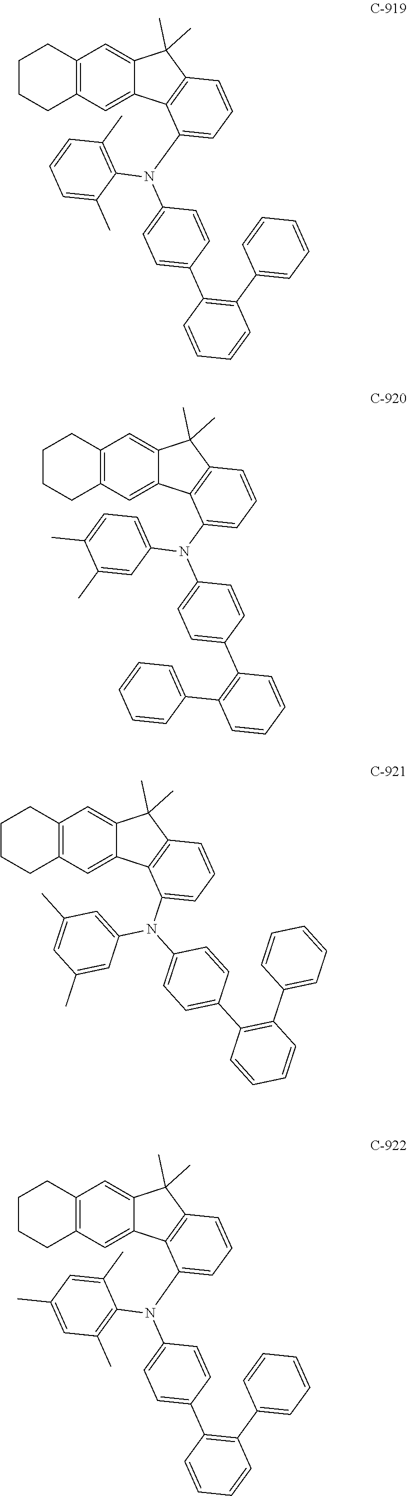 [SMILES: Cc1cccc(C)c1N(c1ccc(-c2ccccc2-c2ccccc2)cc1)c1cccc2c1-c1cc3c(cc1C2(C)C)CCCC3 ; Cc1ccc(N(c2ccc(-c3ccccc3-c3ccccc3)cc2)c2cccc3c2-c2cc4c(cc2C3(C)C)CCCC4)cc1C ; Cc1cc(C)cc(N(c2ccc(-c3ccccc3-c3ccccc3)cc2)c2cccc3c2-c2cc4c(cc2C3(C)C)CCCC4)c1 ; Cc1cc(C)c(N(c2ccc(-c3ccccc3-c3ccccc3)cc2)c2cccc3c2-c2cc4c(cc2C3(C)C)CCCC4)c(C)c1]

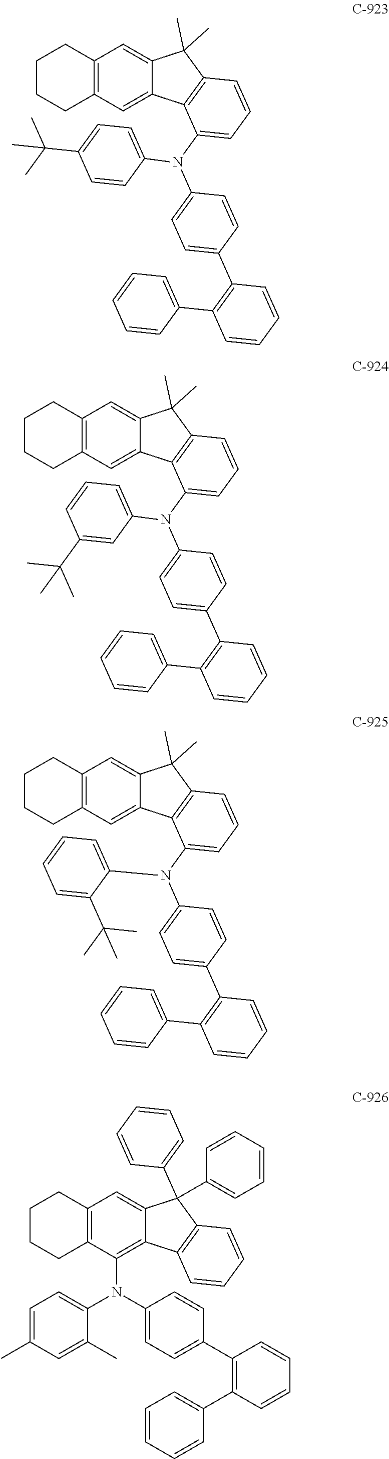

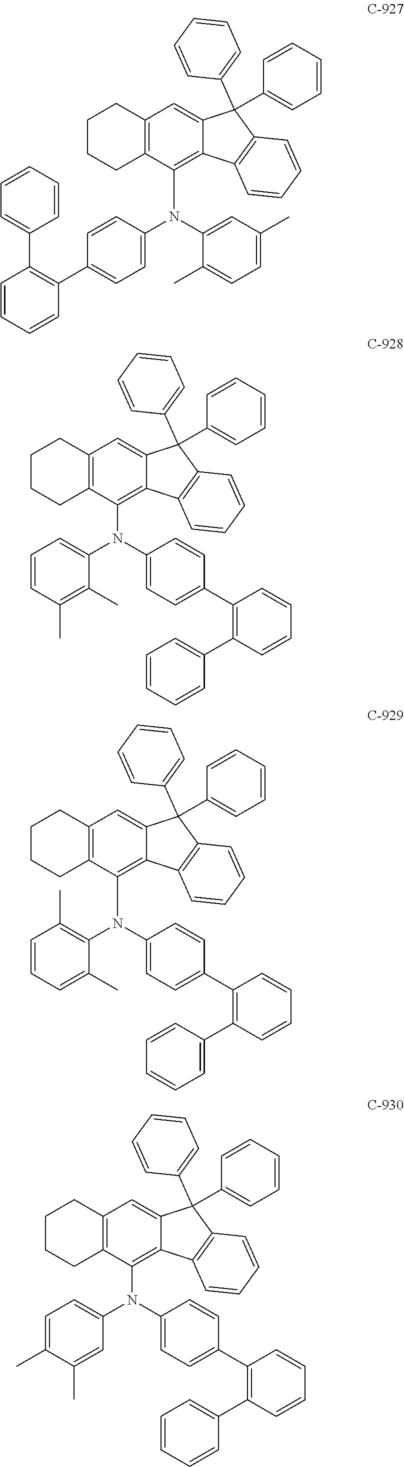

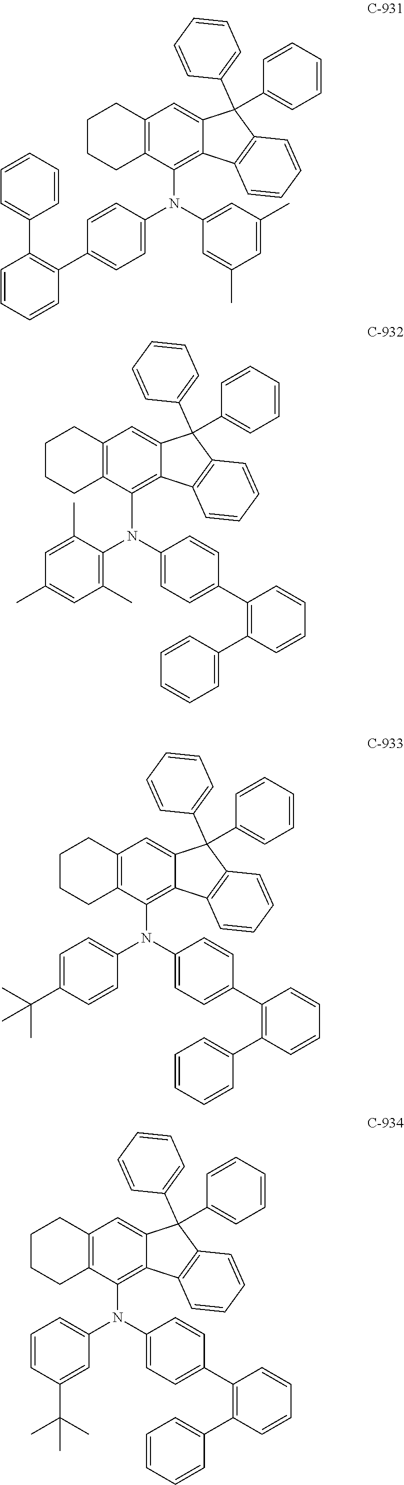

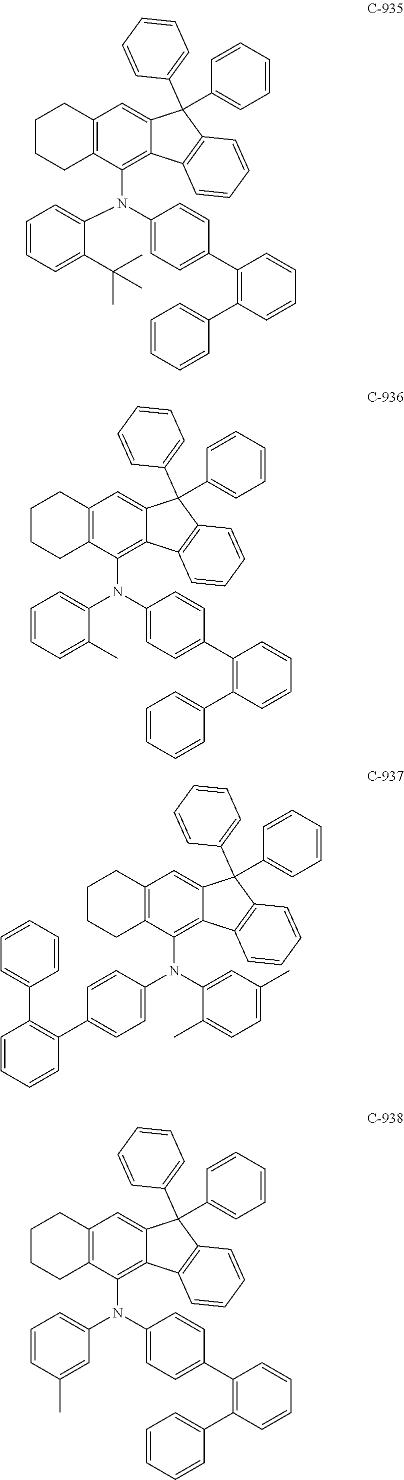

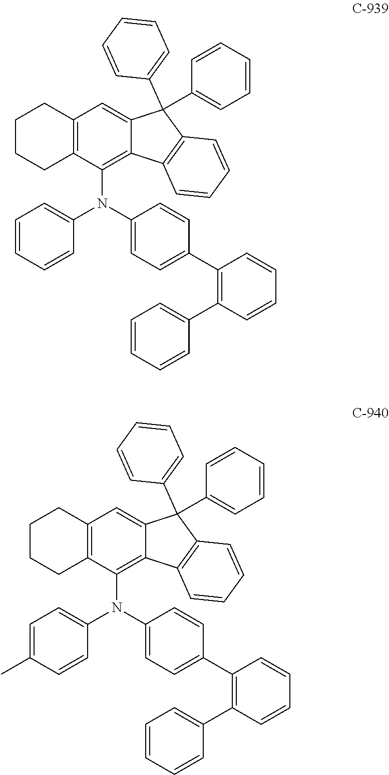 [SMILES: c1ccc(-c2ccccc2-c2ccc(N(c3ccccc3)c3c4c(cc5c3-c3ccccc3C5(c3ccccc3)c3ccccc3)CCCC4)cc2)cc1 ; Cc1ccc(N(c2ccc(-c3ccccc3-c3ccccc3)cc2)c2c3c(cc4c2-c2ccccc2C4(c2ccccc2)c2ccccc2)CCCC3)cc1]

The compounds represented by Formula 1 according to the present disclosure may be produced by synthetic methods known to one skilled in the art. For example, the compounds of the present disclosure may be synthesized by referring to Korean Patent Application Laid-Open Nos. 2023-0034139 (Mar. 9, 2023) and 2021-0143521 (Nov. 29, 2021), but are not limited thereto.

Hereinafter, an organic electroluminescent device employing the above-described organic electroluminescent compound will be described.

An organic electroluminescent device according to one embodiment of the present disclosure comprises a first electrode; a second electrode; and one or more of organic layers positioned between the first electrode and the second electrode, wherein the organic layers comprise a hole transport layer, a light-emitting layer, a hole auxiliary layer, an electron-blocking layer, and an emission auxiliary layer. According to one example, at least one of the hole transport layer, the light-emitting layer, the hole auxiliary layer, the electron-blocking layer, and the emission auxiliary layer may comprise an organic electroluminescent compound represented by Formula 1. For example, the hole transport layer may comprise an organic electroluminescent compound represented by Formula 1. According to one example, an organic electroluminescent material of the present disclosure may comprise at least one compound among compounds C-1 to C-940, and an organic electroluminescent material may be comprised in the same organic layer, for example, the hole transport layer.

A light-emitting layer according to one example may comprise a first host compound and a second host compound. Here, the weight ratio of the first host compound to the second host compound may be included in the light-emitting layer in a range of about 1:99 to about 99:1, preferably about 10:90 to about 90:10, more preferably about 30:70 to about 70:30, more preferably about 40:60 to about 60:40, and even more preferably about 50:50.

An organic electroluminescent device according to one embodiment of the present disclosure comprises a first electrode; a second electrode facing the first electrode; a light-emitting layer between the first electrode and the second electrode, and a hole transport zone between the first electrode and the light-emitting layer, wherein the hole transport zone comprises the compound represented by Formula 1, and the light-emitting layer comprises a compound represented by the following Formula 6 and a compound represented by the following Formula 7.

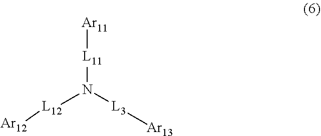

In Formula 6, L₁₁ to L₁₃ each independently represent a single bond, a substituted or unsubstituted (C6-C30)arylene, or a substituted or unsubstituted (3- to 30-membered)heteroarylene. According to one embodiment of the present disclosure, L₁₁ to L₁₃ each independently represent a single bond, a substituted or unsubstituted (C6-C20)arylene, or a substituted or unsubstituted (3- to 20-membered)heteroarylene. For example, L₁₁ to L₁₃ may each independently be a single bond, a phenylene unsubstituted or substituted with deuterium, a biphenylene unsubstituted or substituted with deuterium, a terphenylene unsubstituted or substituted with deuterium, a naphthylene unsubstituted or substituted with deuterium, a phenanthrenylene, a dibenzofuranylene, a dibenzothiophenylene, a carbazolylene, or a pyridylene, and these may be further substituted with deuterium.

In Formula 6, Ar₁₁ represents a substituted or unsubstituted (3- to 30-membered)heteroaryl. According to one embodiment of the present disclosure, Ar₁₁ represents a substituted or unsubstituted (3- to 25-membered)heteroaryl. According to another embodiment of the present disclosure, Ar₁₁ may be a substituted or unsubstituted (3- to 30-membered)heteroaryl having four or more rings. According to yet another embodiment of the present disclosure, Ar₁₁ may be a substituted or unsubstituted (3- to 30-membered)heteroaryl in which four or more rings are fused and which necessarily contains an oxygen atom. According to still another embodiment of the present disclosure, Ar₁₁ may be represented by the following Formulas 6-1 or 6-2.

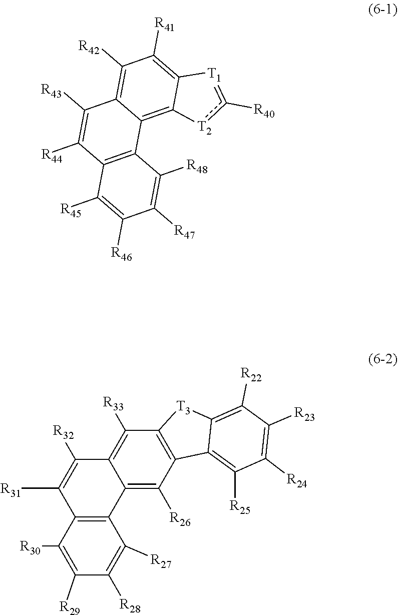

In Formulas 6-1 and 6-2, T₁ and T₂ each independently represent —N═, —NR₂₀—, —O—, or —S—, with the proviso that one of T₁ and T₂ is —N═, and the other of T₁ and T₂ is —NR₂₀—, —O—, or —S—. For example, T₁ and T₂ may each independently be —N═, —O—, or —S—.

In Formula 6-2, T₃ represents 0 or S. For example, T₃ may be 0.

In Formula 6-1, R₄₀ represents a substituted or unsubstituted (C6-C30)aryl, or a substituted or unsubstituted (3- to 30-membered)heteroaryl. According to one embodiment of the present disclosure, Rao represents a substituted or unsubstituted (C6-C15)aryl, or a substituted or unsubstituted (3- to 10-membered)heteroaryl. For example, Rao may be a phenyl unsubstituted or substituted with deuterium, a naphthyl, a biphenyl, or a pyridyl, and these may be further substituted with deuterium.

In Formulas 6-1 and 6-2, R₄₁ to R₄₈ and R₂₂ to R₃₃ are each independently linked to L₁₁, or represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di(C1-C30)alkylamino, a substituted or unsubstituted mono- or di(C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3-to 30-membered)heteroarylamino, a substituted or unsubstituted mono- or di(C6-C30)arylamino, a substituted or unsubstituted mono- or di(3- to 30-membered)heteroarylamino, or a substituted or unsubstituted (C6-C30)aryl(3- to 30-membered)heteroarylamino, or may be linked to an adjacent substituent(s) to form a ring(s), with the proviso that in Formula 6-1, any one of R₄₁ to R₄₈ is linked to L₁₁, and in Formula 6-2, any one of R₂₂ to R₃₃ is linked to L₁₁. According to one embodiment of the present disclosure, R₄₁ to R₄₈ and R₂₂ to R₃₃ each independently represent hydrogen, deuterium, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted fused ring group of a (C3-C30) aliphatic ring(s) and a (C6-C30) aromatic ring(s), a substituted or unsubstituted (C1-C30)alkyl(C6-C30)arylamino, a substituted or unsubstituted (C1-C30)alkyl(3- to 30-membered)heteroarylamino, a substituted or unsubstituted mono- or di(C6-C30)arylamino, a substituted or unsubstituted mono- or di(3- to 30-membered)heteroarylamino, or a substituted or unsubstituted (C6-C30)aryl(3- to 30-membered)heteroarylamino, or may be linked to an adjacent substituent(s) to form a ring(s). According to another embodiment of the present disclosure, R₄₁ to R₄₈ and R₂₂ to R₃₃ each independently represent hydrogen, deuterium, a substituted or unsubstituted (C1-C30)alkyl, a substituted or unsubstituted (C6-C30)aryl, or a substituted or unsubstituted (3- to 30-membered)heteroaryl. For example, R₄₁ to R₄₈ and R₂₂ to R₃₃ may each independently be hydrogen or deuterium.

In Formula 6, Ar₁₂ and Ar₁₃ each independently represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a mono- or di(C1-C30)alkylamino, a mono- or di(C2-C30)alkenylamino, a substituted or unsubstituted (C1-C30)alkyl(C2-C30)alkenylamino, a substituted or unsubstituted mono- or di(C6-C30)arylamino, a substituted or unsubstituted (C1-C30)alkyl(C6-C30)arylamino, a substituted or unsubstituted mono- or di(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino. According to one embodiment of the present disclosure, Ar₁₂ and Ar₁₃ each independently represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted mono- or di(C6-C30)arylamino, or a substituted or unsubstituted (C6-C30)aryl(3- to 30-membered)heteroarylamino. According to another embodiment of the present disclosure, Ar₁₂ and Ar₁₃ each independently represent a substituted or unsubstituted (C6-C30)aryl, a substituted or unsubstituted (3- to 30-membered)heteroaryl, a substituted or unsubstituted mono- or di(C6-C30)arylamino, or a substituted or unsubstituted (C6-C30)aryl(3- to 30-membered)heteroarylamino. For example, Ar₁₂ and Ar₁₃ may each independently be a phenyl unsubstituted or substituted with deuterium, cyano, a phenyl unsubstituted or substituted with deuterium, a biphenyl, a naphthyl unsubstituted or substituted with deuterium, a phenanthrenyl, a dibenzofuranyl, or a carbazolyl; a biphenyl unsubstituted or substituted with deuterium or a phenyl; a naphthyl unsubstituted or substituted with deuterium, a phenyl unsubstituted or substituted with deuterium, or a naphthyl unsubstituted or substituted with a pyridyl; a phenanthrenyl unsubstituted or substituted with deuterium, a phenyl or a pyridyl; a dimethylfluorenyl; a dimethylbenzofluorenyl; a diphenylfluorenyl unsubstituted or substituted with deuterium; an o-terphenyl unsubstituted or substituted with deuterium; a m-terphenyl; a p-terphenyl unsubstituted or substituted with deuterium; a 2,6-dimethylphenyl; a tert-butyl phenyl; a fluoranthenyl; an anthracenyl; a spirobifluorenyl; a quaterphenyl unsubstituted or substituted with deuterium; a triphenylenyl unsubstituted or substituted with deuterium; a dibenzofuranyl unsubstituted or substituted with deuterium, a phenyl, or a pyridyl; a dibenzothiophenyl unsubstituted or substituted with deuterium, or a phenyl; a pyridyl unsubstituted or substituted with a phenyl; a benzonaphthofuranyl; a benzonaphthothiophenyl; a carbazolyl unsubstituted or substituted with a phenyl or a biphenyl; a phenoxazinyl; a benzimidazolyl unsubstituted or substituted with a phenyl; a triphenylsilyl; a dibenzoselenophenyl; a 14-membered heteroaryl substituted with a methyl; a 22-membered heteroaryl; a benzophenanthrenyl; a benzonaphthoselenophenyl; a diphenyl amino; a phenylbiphenyl amino; a phenyl dibenzofuranyl amino; a phenyl dibenzothiophenyl amino; or a phenyl pyridyl amino, and these may be further substituted with deuterium.

The organic electroluminescent compound represented by Formula 6 may be selected from the following compounds, but is not limited thereto.

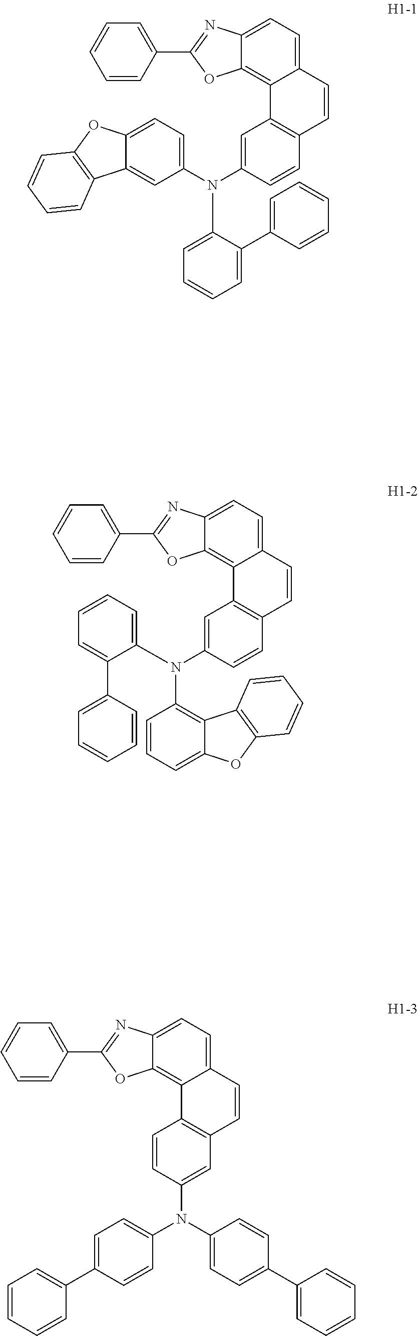 [SMILES: c1ccc(-c2nc3ccc4ccc5ccc(N(c6ccc7oc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

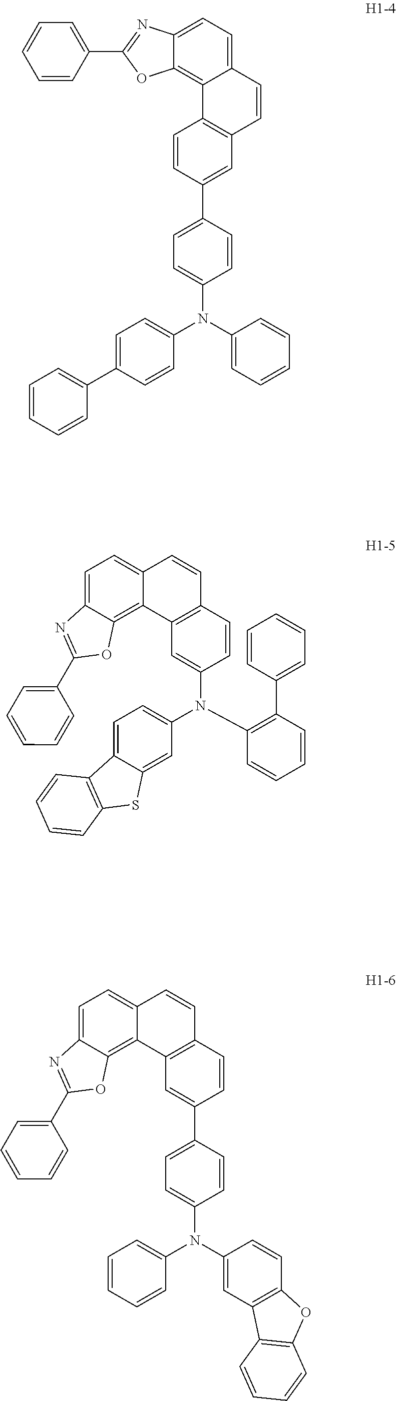 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

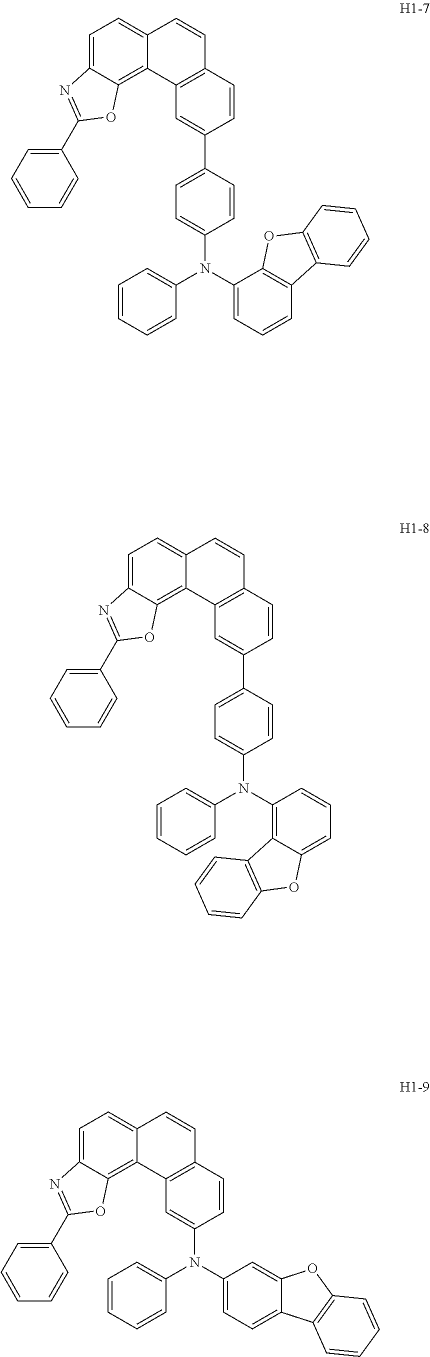 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccccc6)c6ccc7c(c6)oc6ccccc67)cc5c4c3o2)cc1]

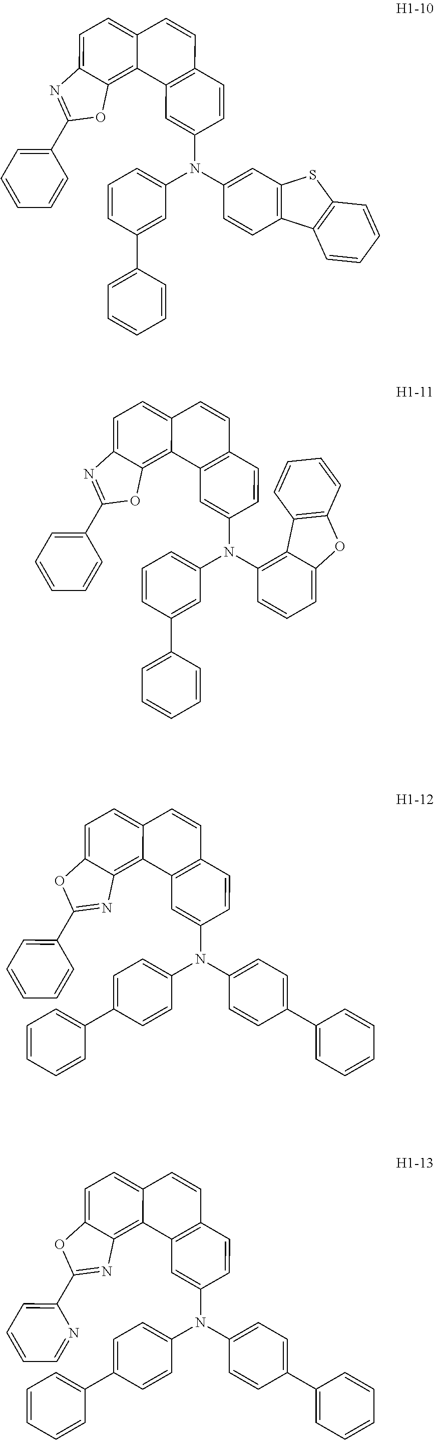 [SMILES: c1ccc(-c2cccc(N(c3ccc4c(c3)sc3ccccc34)c3ccc4ccc5ccc6nc(-c7ccccc7)oc6c5c4c3)c2)cc1 ; c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccn7)nc6c5c4c3)cc2)cc1]

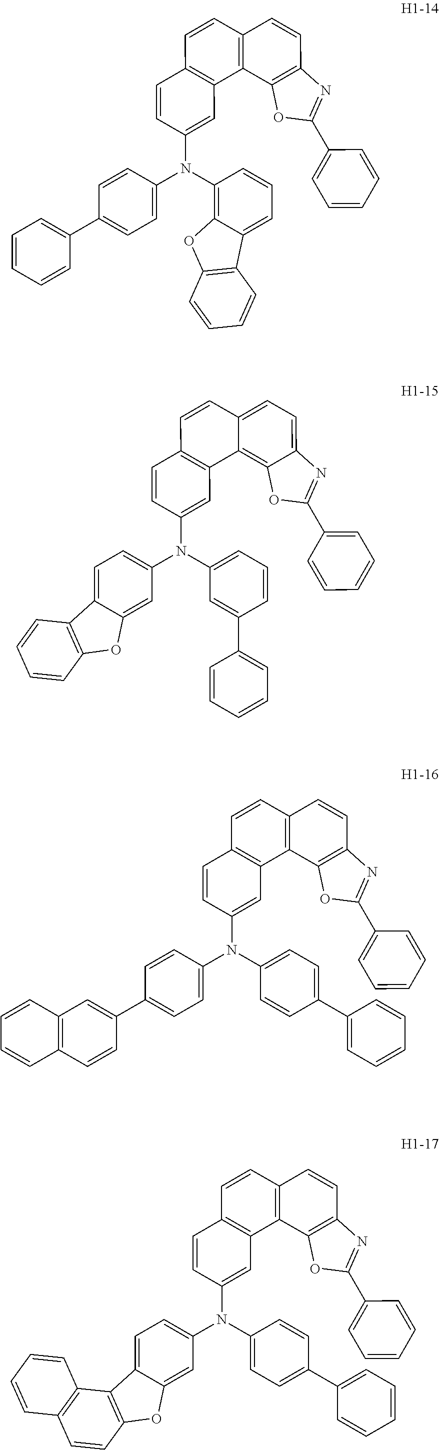 [SMILES: c1ccc(-c2ccc(N(c3ccc4ccc5ccc6nc(-c7ccccc7)oc6c5c4c3)c3cccc4c3oc3ccccc34)cc2)cc1 ; c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1]

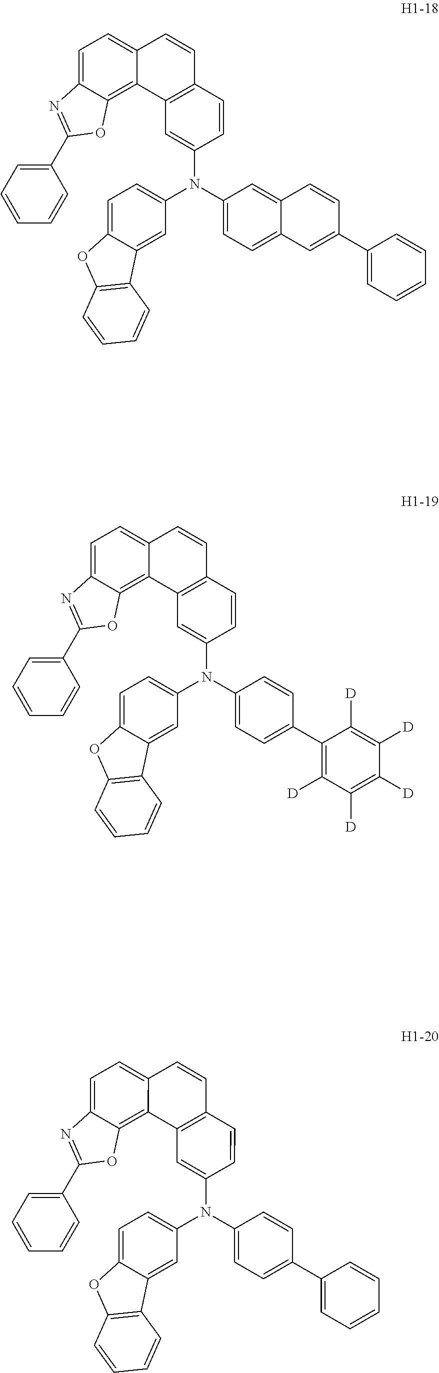 [SMILES: c1ccc(-c2ccc3cc(N(c4ccc5oc6ccccc6c5c4)c4ccc5ccc6ccc7nc(-c8ccccc8)oc7c6c5c4)ccc3c2)cc1 ; [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2ccc(N(c3ccc4oc5ccccc5c4c3)c3ccc4ccc5ccc6nc(-c7ccccc7)oc6c5c4c3)cc2)cc1]

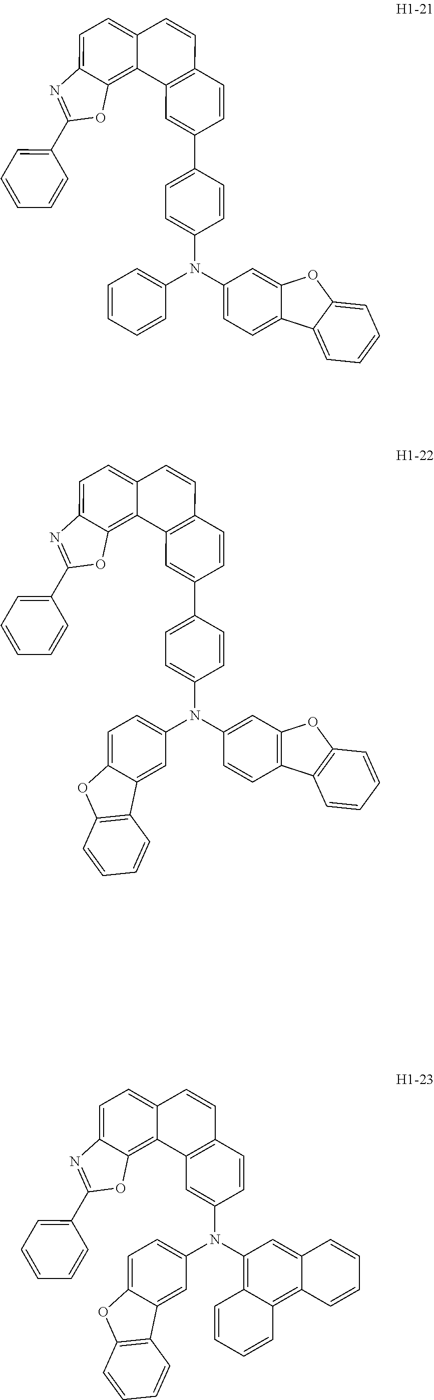 [SMILES: c1ccc(-c2nc3ccc4ccc5ccc(-c6ccc(N(c7ccccc7)c7ccc8c(c7)oc7ccccc78)cc6)cc5c4c3o2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1]

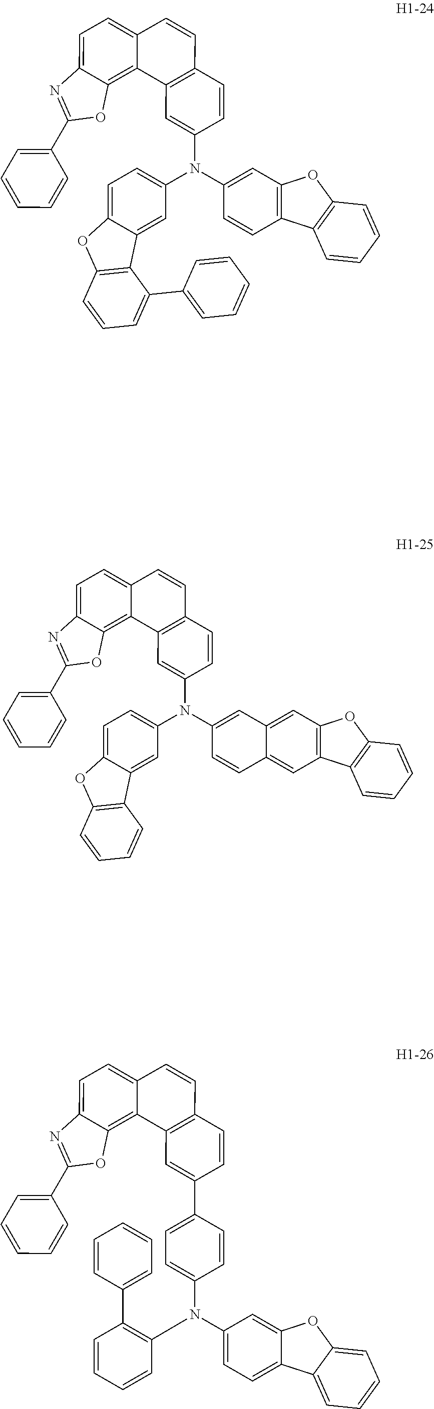 [SMILES: c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1]

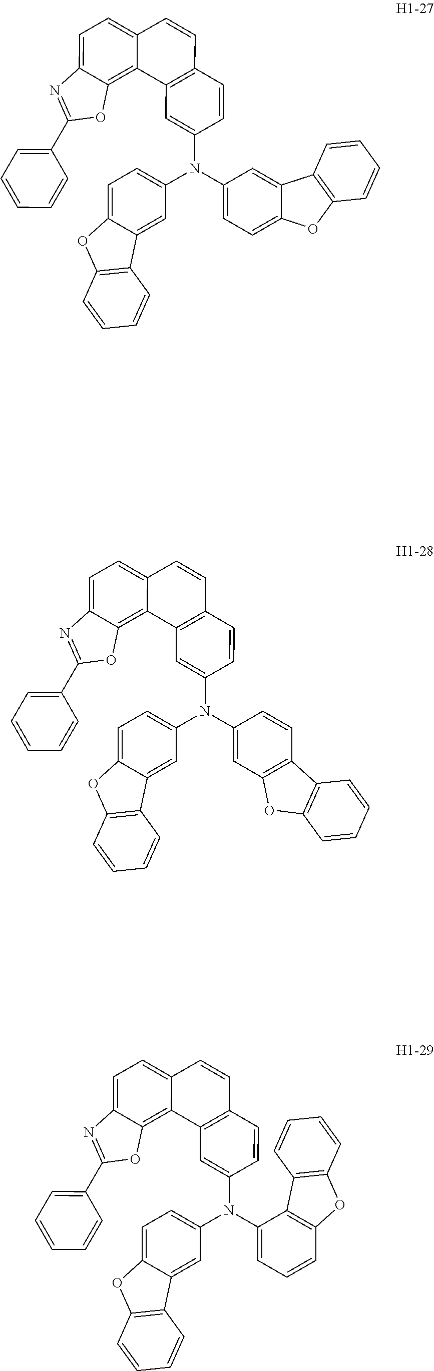 [SMILES: c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1]

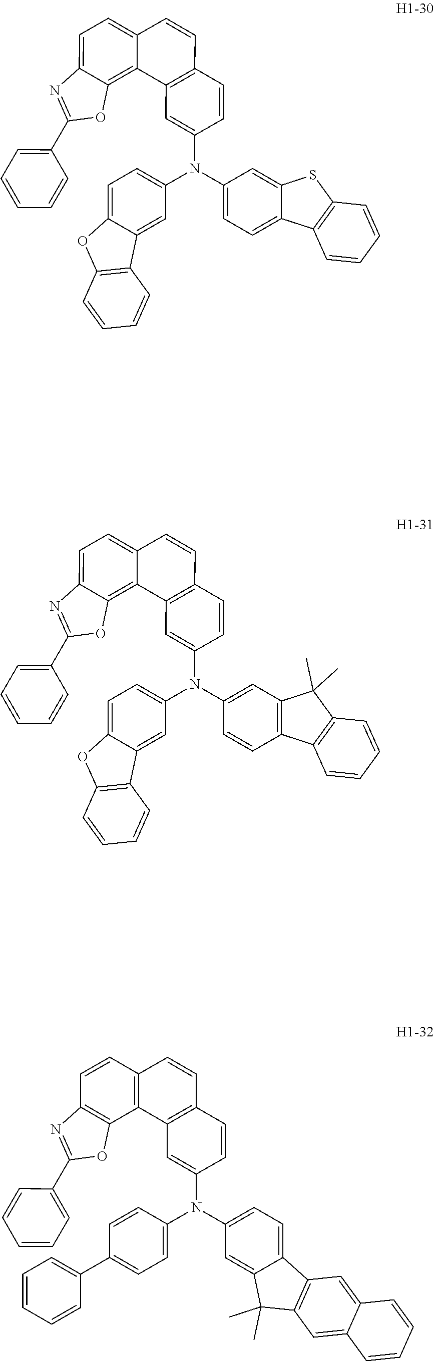 [SMILES: c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21]

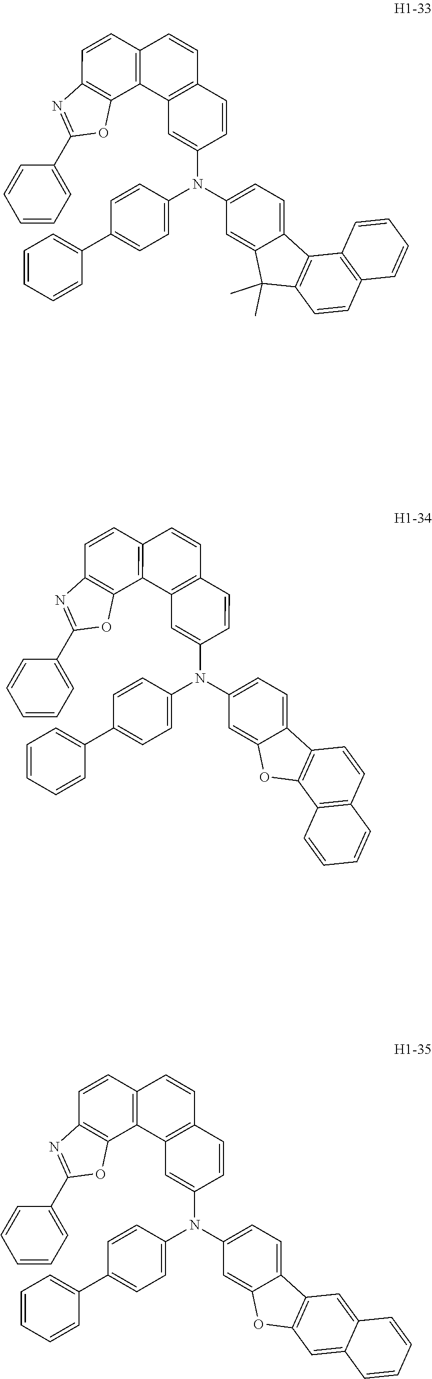 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; c1ccc(-c2ccc(N(c3ccc4c(c3)oc3cc5ccccc5cc34)c3ccc4ccc5ccc6nc(-c7ccccc7)oc6c5c4c3)cc2)cc1]

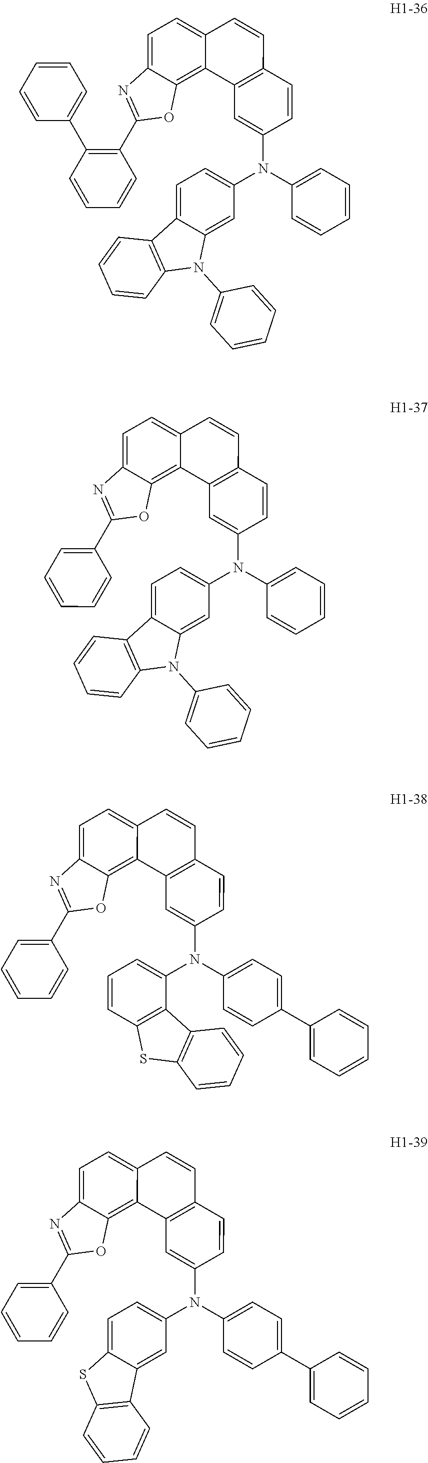 [SMILES: c1ccc(-c2ccccc2-c2nc3ccc4ccc5ccc(N(c6ccccc6)c6ccc7c8ccccc8n(-c8ccccc8)c7c6)cc5c4c3o2)cc1 ; c1ccc(-c2nc3ccc4ccc5ccc(N(c6ccccc6)c6ccc7c8ccccc8n(-c8ccccc8)c7c6)cc5c4c3o2)cc1 ; c1ccc(-c2ccc(N(c3ccc4ccc5ccc6nc(-c7ccccc7)oc6c5c4c3)c3cccc4sc5ccccc5c34)cc2)cc1 ; c1ccc(-c2ccc(N(c3ccc4sc5ccccc5c4c3)c3ccc4ccc5ccc6nc(-c7ccccc7)oc6c5c4c3)cc2)cc1]

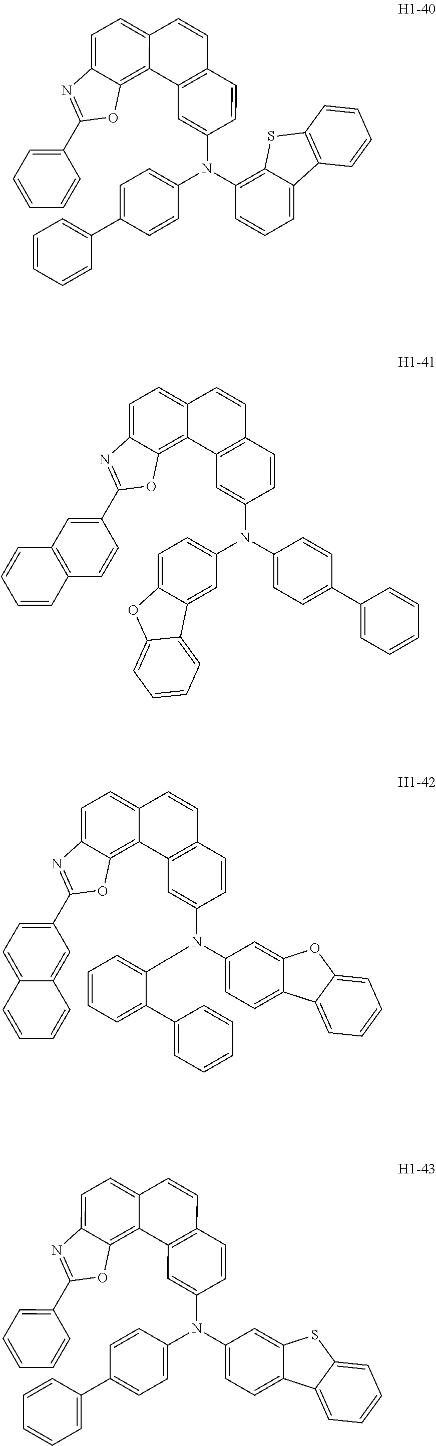

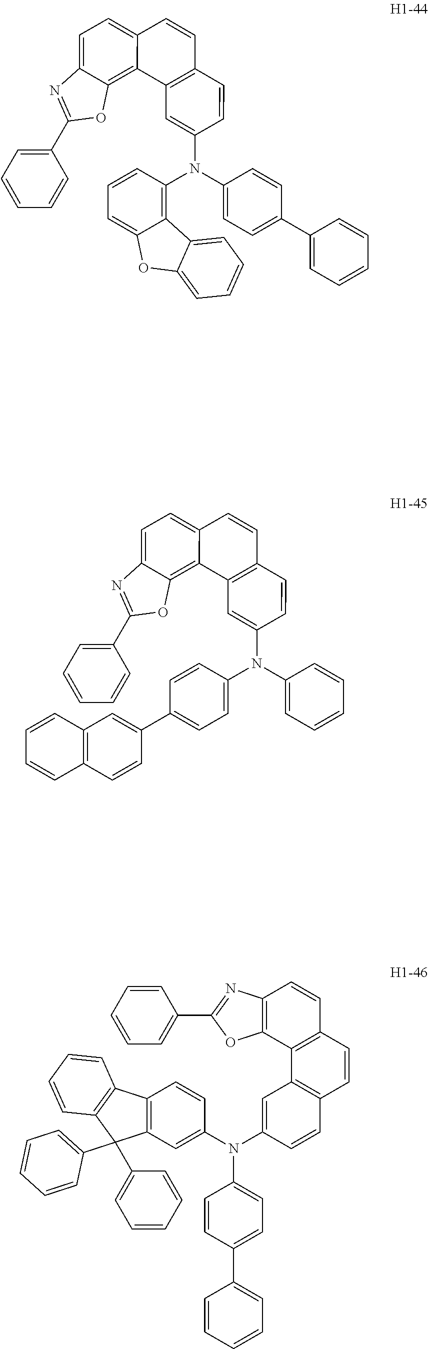 [SMILES: c1ccc(-c2ccc(N(c3ccc4ccc5ccc6nc(-c7ccccc7)oc6c5c4c3)c3cccc4oc5ccccc5c34)cc2)cc1 ; c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4c(c3)C(c3ccccc3)(c3ccccc3)c3ccccc3-4)c3ccc4ccc5ccc6nc(-c7ccccc7)oc6c5c4c3)cc2)cc1]

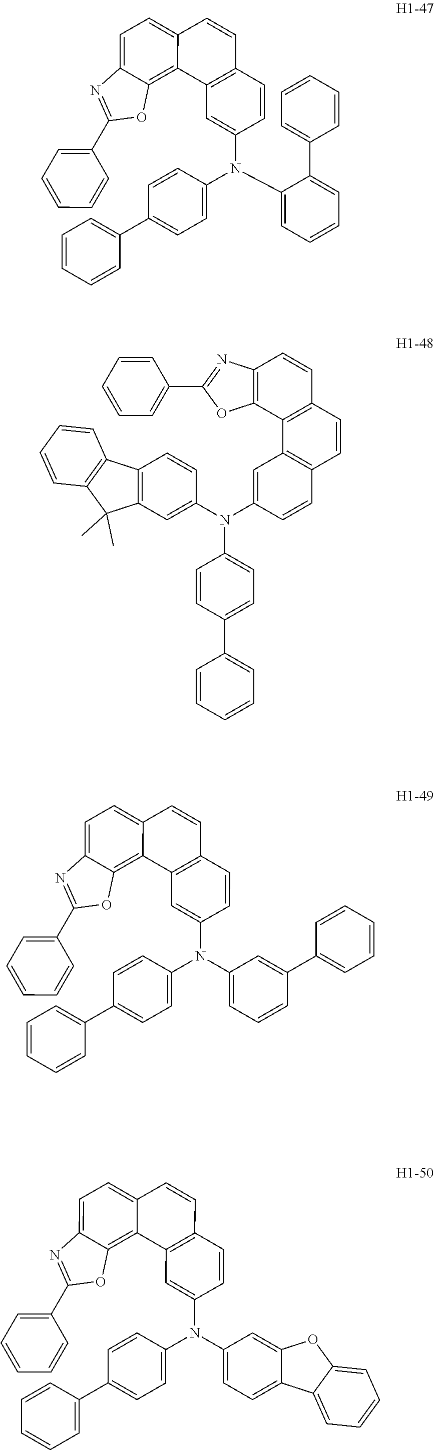 [SMILES: c1ccc(-c2ccc(N(c3ccc4ccc5ccc6nc(-c7ccccc7)oc6c5c4c3)c3ccccc3-c3ccccc3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3cccc(-c4ccccc4)c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1]

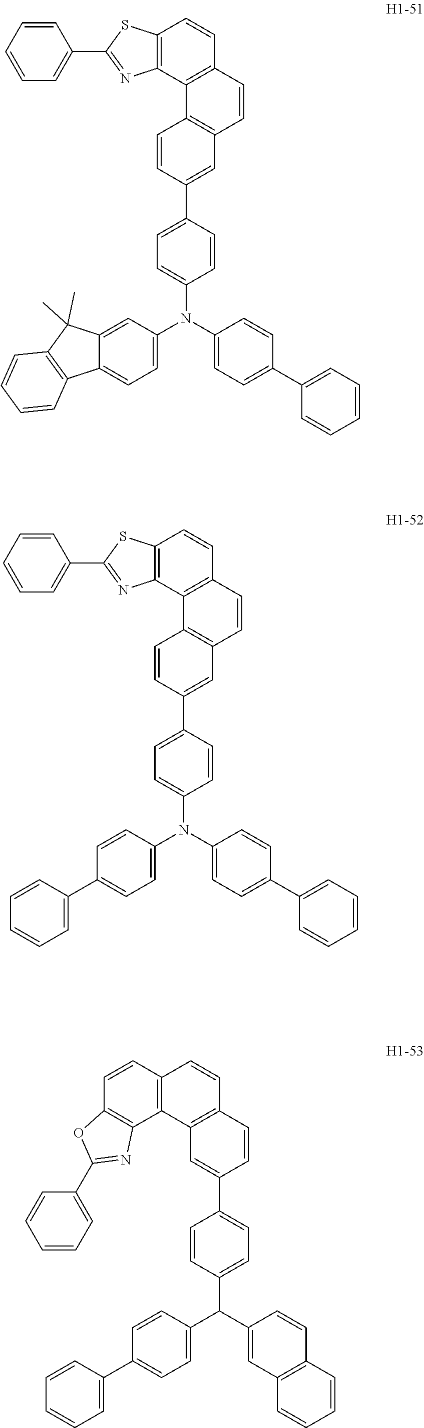 [SMILES: CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; c1ccc(-c2ccc(C(c3ccc(-c4ccc5ccc6ccc7oc(-c8ccccc8)nc7c6c5c4)cc3)c3ccc4ccccc4c3)cc2)cc1]

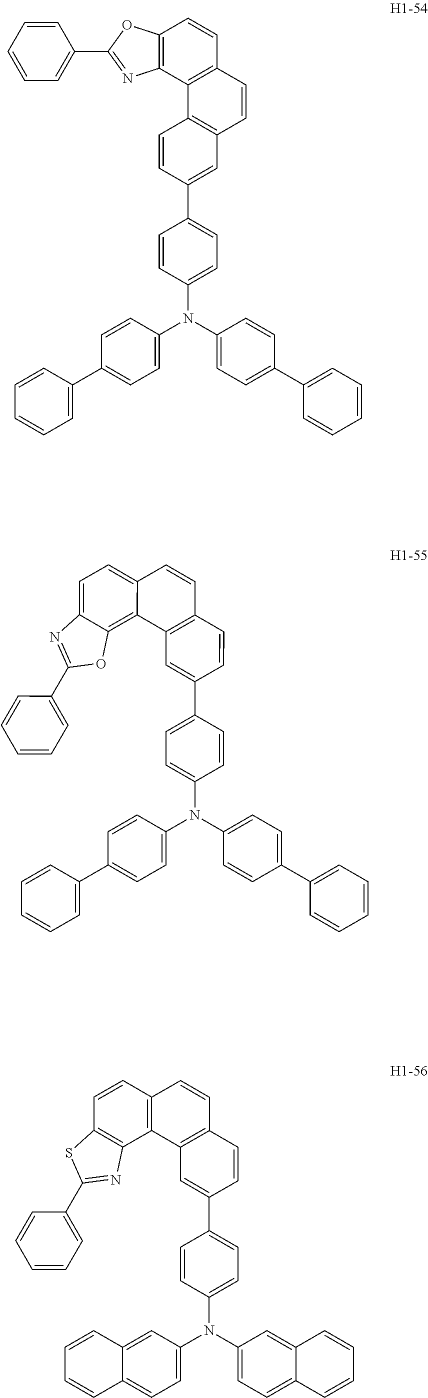 [SMILES: c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2nc3c(ccc4ccc5ccc(-c6ccc(N(c7ccc8ccccc8c7)c7ccc8ccccc8c7)cc6)cc5c43)s2)cc1]

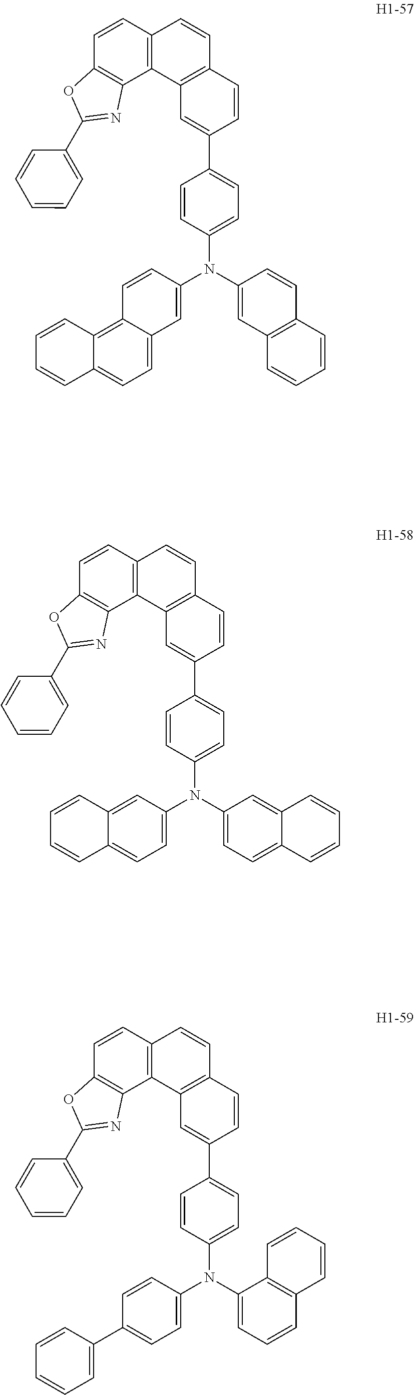 [SMILES: c1ccc(-c2nc3c(ccc4ccc5ccc(-c6ccc(N(c7ccc8ccccc8c7)c7ccc8c(ccc9ccccc98)c7)cc6)cc5c43)o2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2ccc(N(c3ccc(-c4ccc5ccc6ccc7oc(-c8ccccc8)nc7c6c5c4)cc3)c3cccc4ccccc34)cc2)cc1]

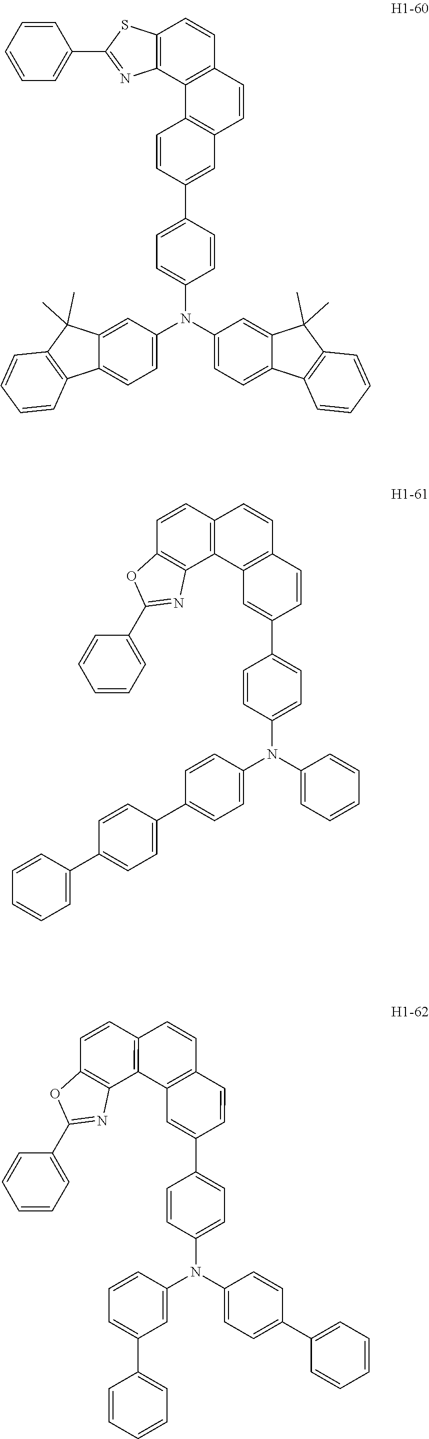 [SMILES: CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1]

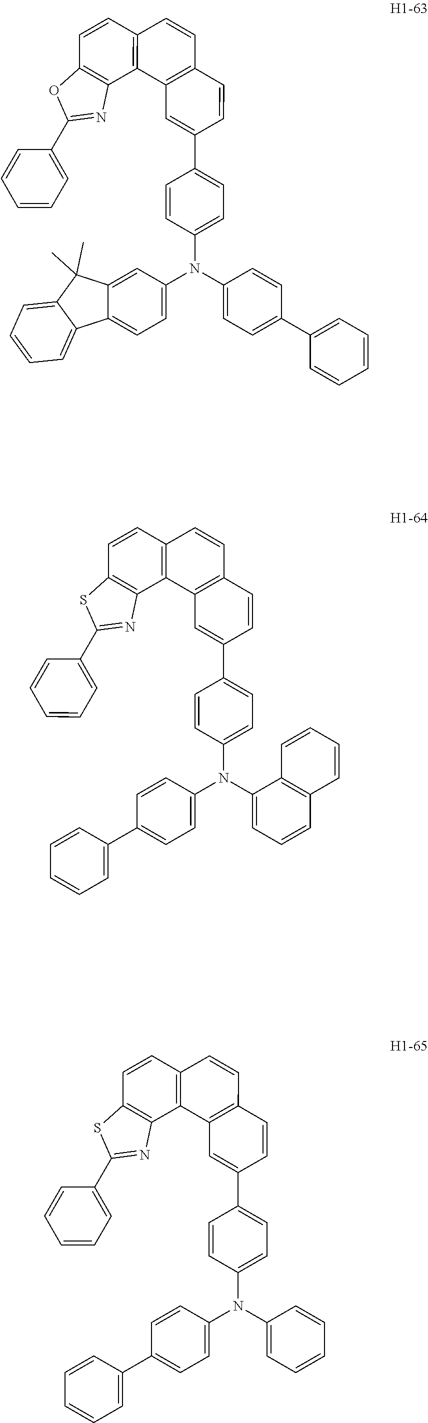 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7sc(-c8ccccc8)nc7c6c5c4)cc3)c3cccc4ccccc34)cc2)cc1 ; c1ccc(-c2ccc(N(c3ccccc3)c3ccc(-c4ccc5ccc6ccc7sc(-c8ccccc8)nc7c6c5c4)cc3)cc2)cc1]

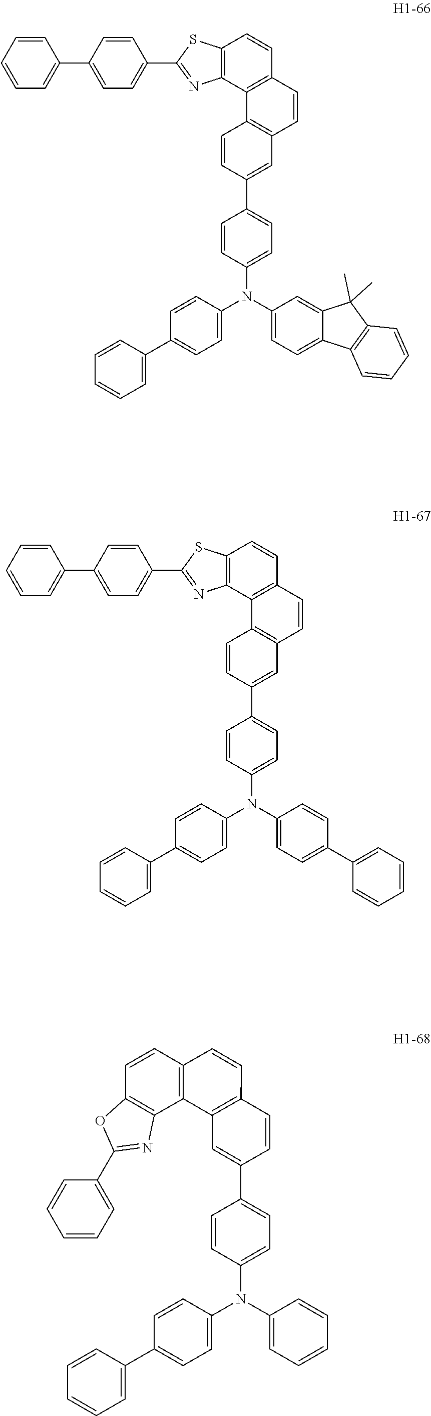 [SMILES: CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; c1ccc(-c2ccc(N(c3ccccc3)c3ccc(-c4ccc5ccc6ccc7oc(-c8ccccc8)nc7c6c5c4)cc3)cc2)cc1]

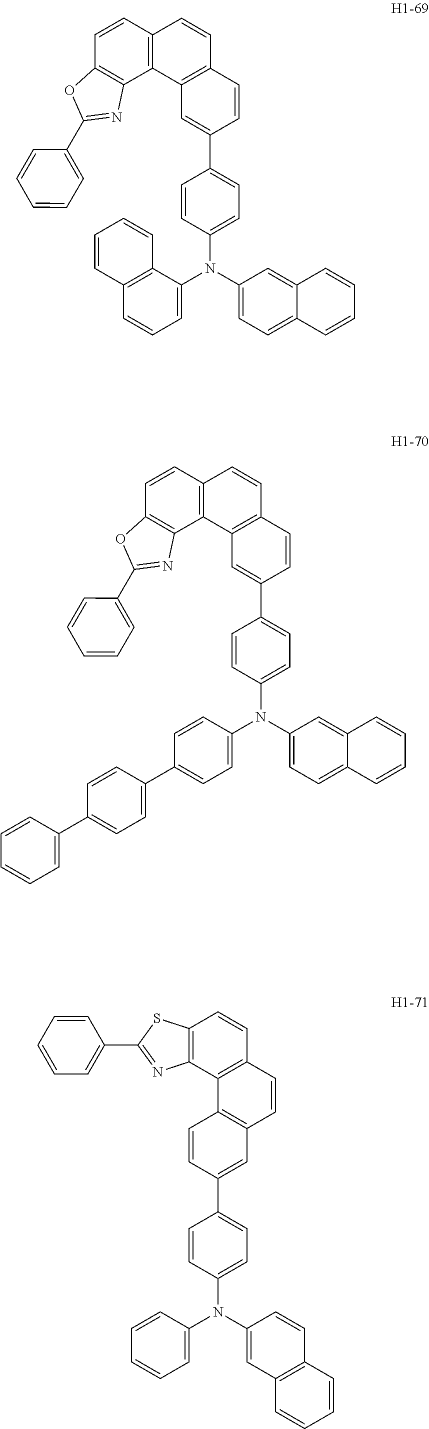 [SMILES: c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5cc(-c6ccc(N(c7ccccc7)c7ccc8ccccc8c7)cc6)ccc5c43)s2)cc1]

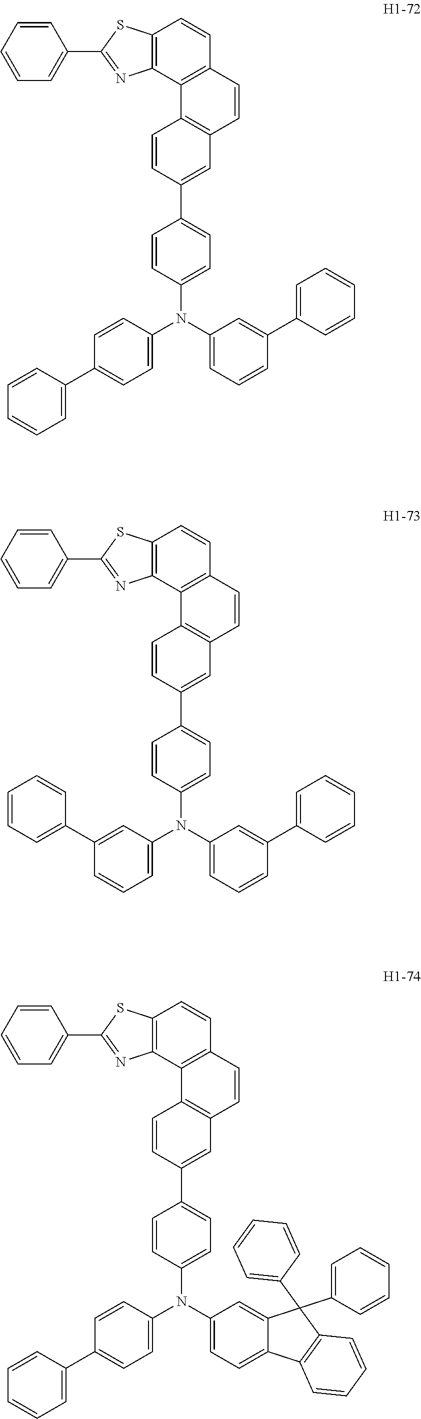 [SMILES: c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1]

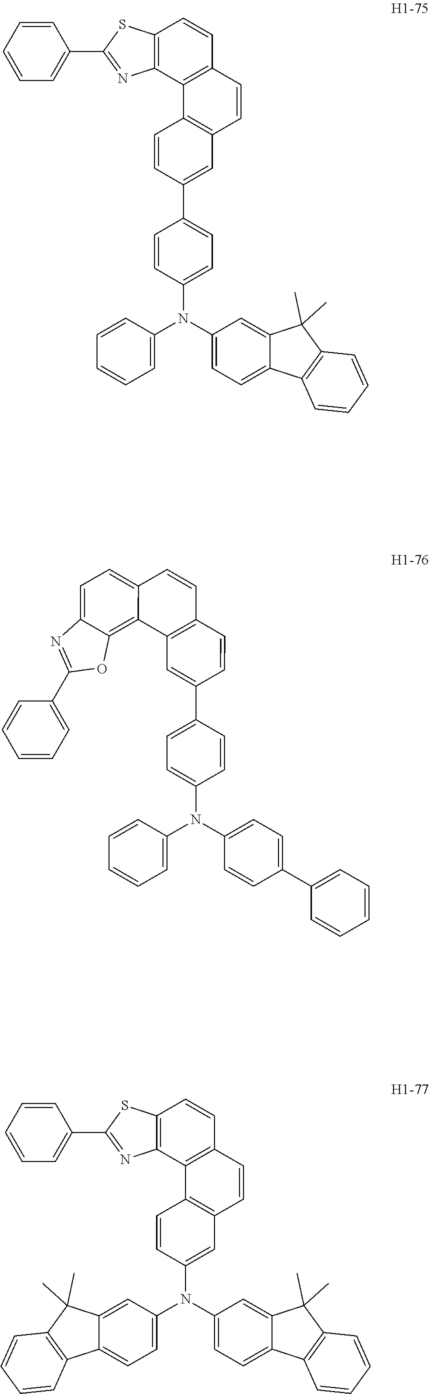 [SMILES: CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccccc3)c3ccc(-c4ccc5ccc6ccc7nc(-c8ccccc8)oc7c6c5c4)cc3)cc2)cc1 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21]

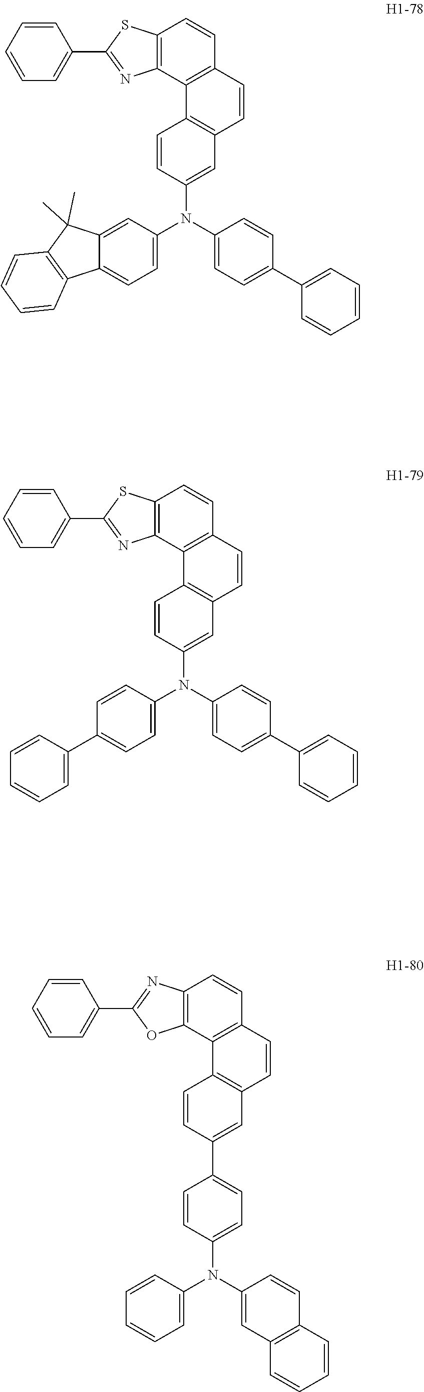 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; c1ccc(-c2ccc(N(c3ccc(-c4ccccc4)cc3)c3ccc4c(ccc5ccc6sc(-c7ccccc7)nc6c54)c3)cc2)cc1 ; c1ccc(-c2nc3ccc4ccc5cc(-c6ccc(N(c7ccccc7)c7ccc8ccccc8c7)cc6)ccc5c4c3o2)cc1]

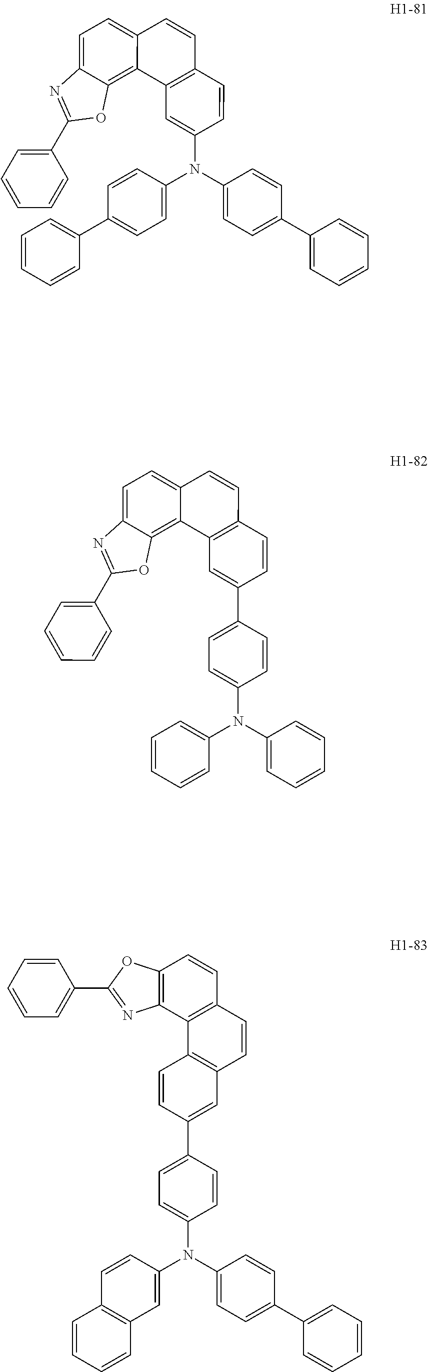 [SMILES: c1ccc(-c2ccc(N(c3ccc(-c4ccccc4)cc3)c3ccc4ccc5ccc6nc(-c7ccccc7)oc6c5c4c3)cc2)cc1 ; c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

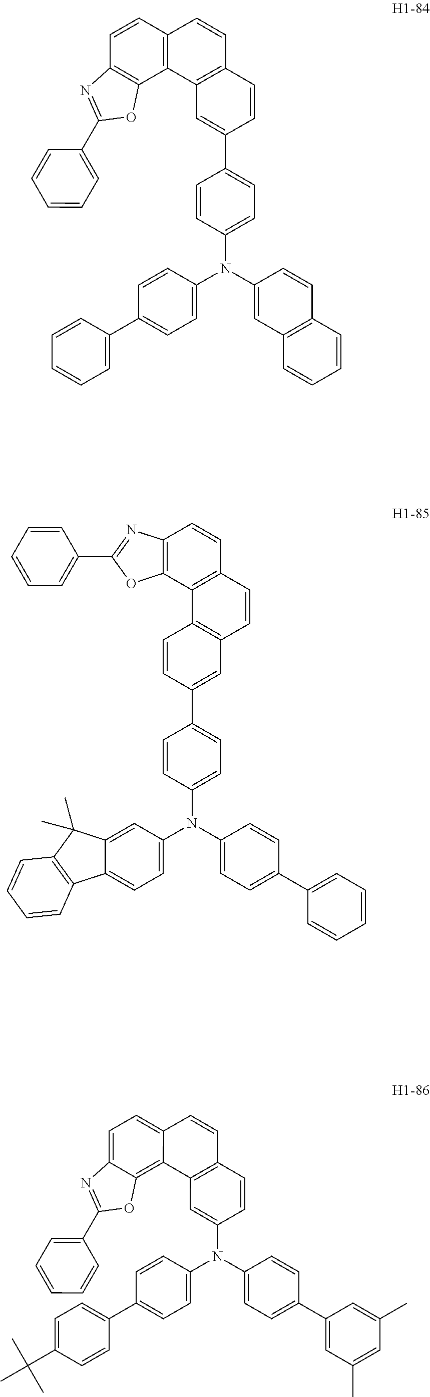 [SMILES: c1ccc(-c2ccc(N(c3ccc(-c4ccc5ccc6ccc7nc(-c8ccccc8)oc7c6c5c4)cc3)c3ccc4ccccc4c3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1]

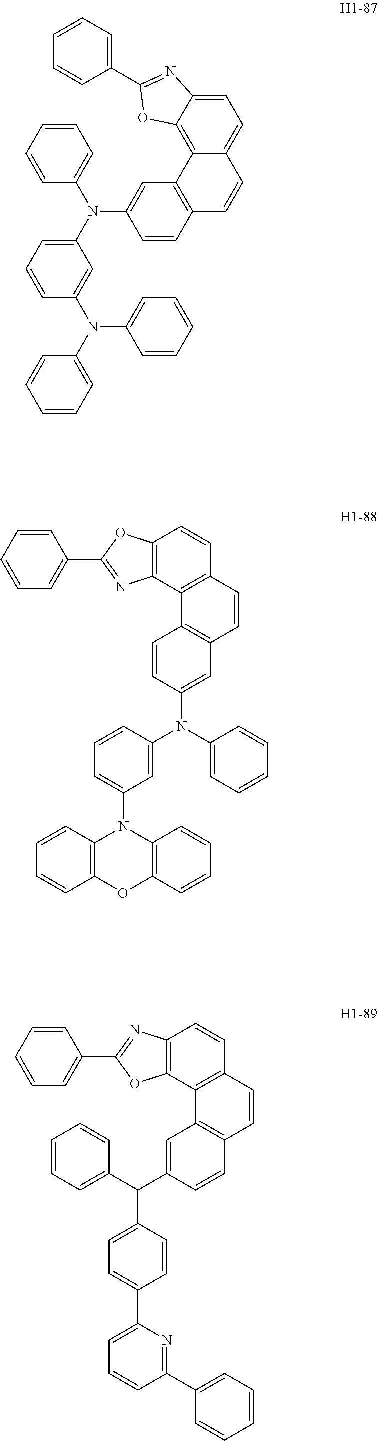 [SMILES: c1ccc(-c2nc3ccc4ccc5ccc(N(c6ccccc6)c6cccc(N(c7ccccc7)c7ccccc7)c6)cc5c4c3o2)cc1 ; c1ccc(-c2nc3c(ccc4ccc5cc(N(c6ccccc6)c6cccc(N7c8ccccc8Oc8ccccc87)c6)ccc5c43)o2)cc1 ; c1ccc(-c2cccc(-c3ccc(C(c4ccccc4)c4ccc5ccc6ccc7nc(-c8ccccc8)oc7c6c5c4)cc3)n2)cc1]

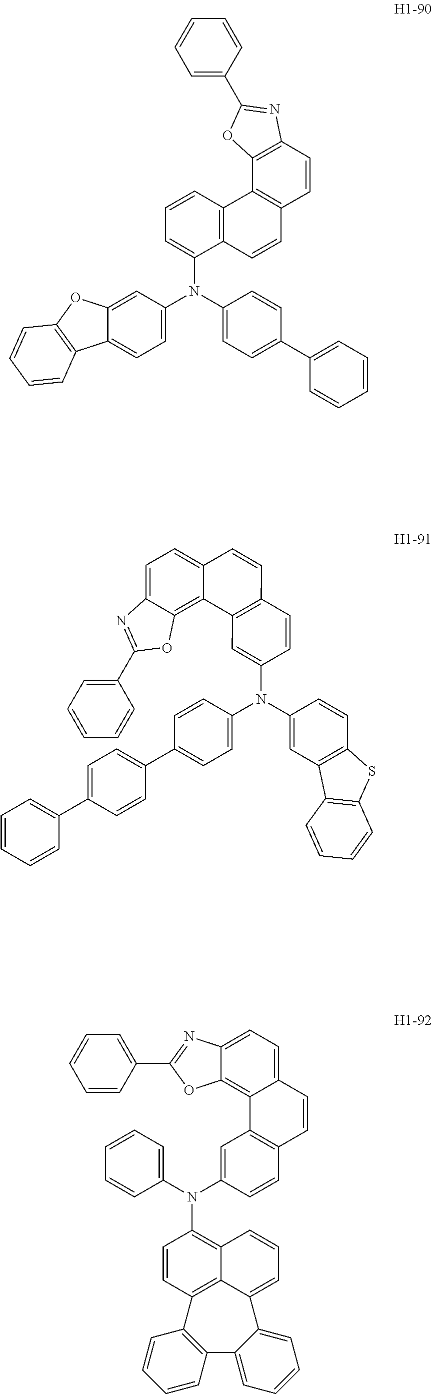 [SMILES: c1ccc(-c2ccc(N(c3ccc4c(c3)oc3ccccc34)c3cccc4c3ccc3ccc5nc(-c6ccccc6)oc5c34)cc2)cc1 ; c1ccc(-c2ccc(-c3ccc(N(c4ccc5sc6ccccc6c5c4)c4ccc5ccc6ccc7nc(-c8ccccc8)oc7c6c5c4)cc3)cc2)cc1 ; c1ccc(-c2nc3ccc4ccc5ccc(N(c6ccccc6)c6ccc7c8c(cccc68)-c6ccccc6-c6ccccc6-7)cc5c4c3o2)cc1]

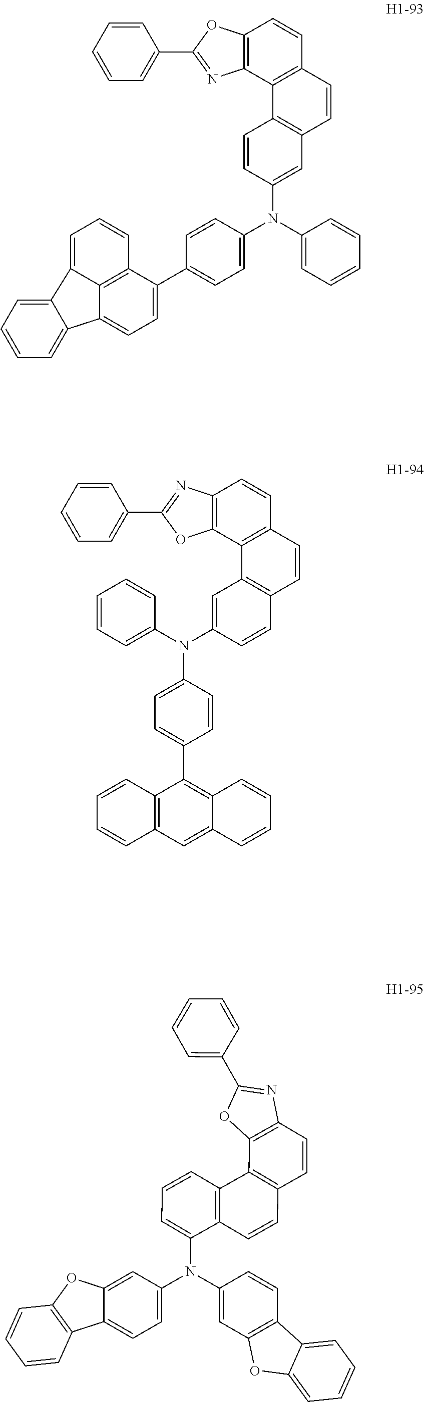 [SMILES: c1ccc(-c2nc3c(ccc4ccc5cc(N(c6ccccc6)c6ccc(-c7ccc8c9c(cccc79)-c7ccccc7-8)cc6)ccc5c43)o2)cc1 ; c1ccc(-c2nc3ccc4ccc5ccc(N(c6ccccc6)c6ccc(-c7c8ccccc8cc8ccccc78)cc6)cc5c4c3o2)cc1 ; c1ccc(-c2nc3ccc4ccc5c(N(c6ccc7c(c6)oc6ccccc67)c6ccc7c(c6)oc6ccccc67)cccc5c4c3o2)cc1]

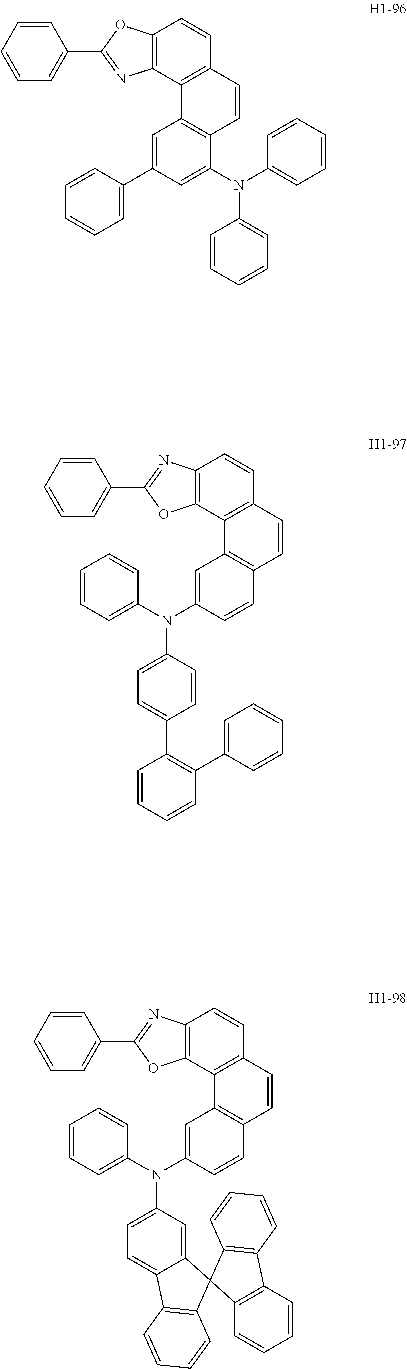 [SMILES: c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2nc3ccc4ccc5ccc(N(c6ccccc6)c6ccc7c(c6)C6(c8ccccc8-c8ccccc86)c6ccccc6-7)cc5c4c3o2)cc1]

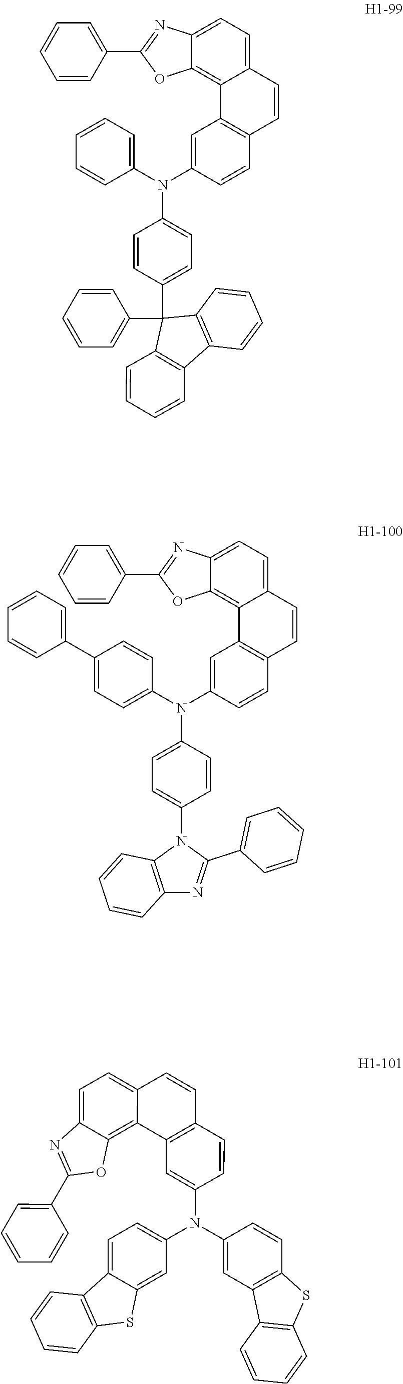 [SMILES: c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1 ; c1ccc(-c2nc3ccc4ccc5ccc(N(c6ccc7c(c6)sc6ccccc67)c6ccc7sc8ccccc8c7c6)cc5c4c3o2)cc1]

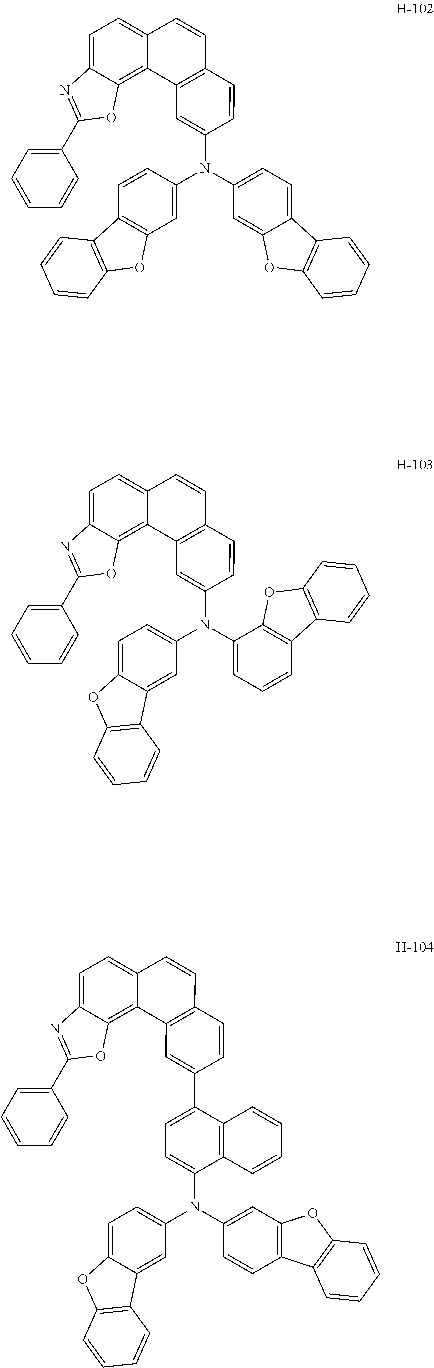 [SMILES: c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1]

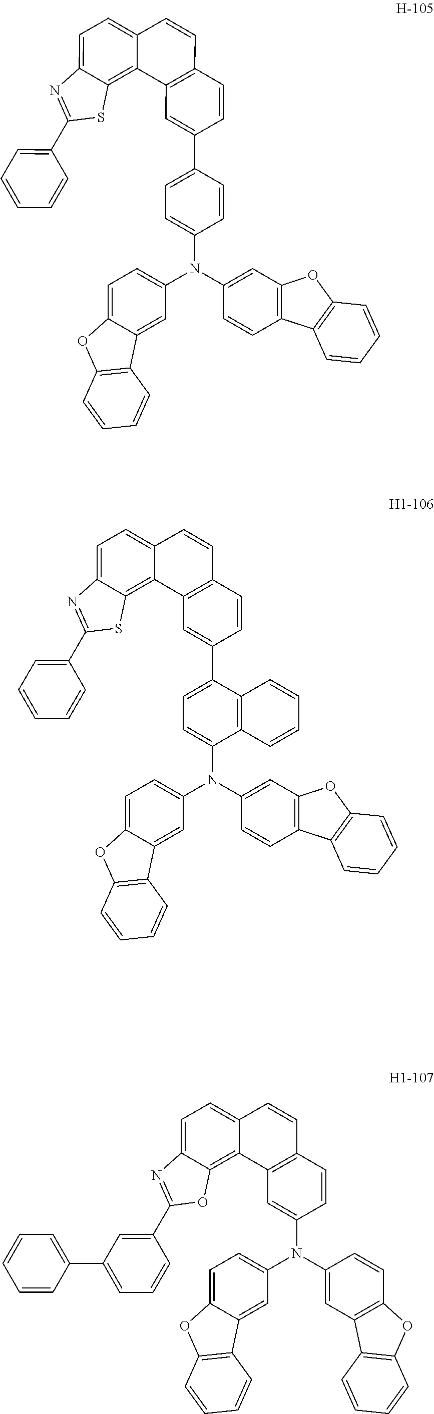 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3s2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3s2)cc1 ; c1ccc(-c2cccc(-c3nc4ccc5ccc6ccc(N(c7ccc8oc9ccccc9c8c7)c7ccc8oc9ccccc9c8c7)cc6c5c4o3)c2)cc1]

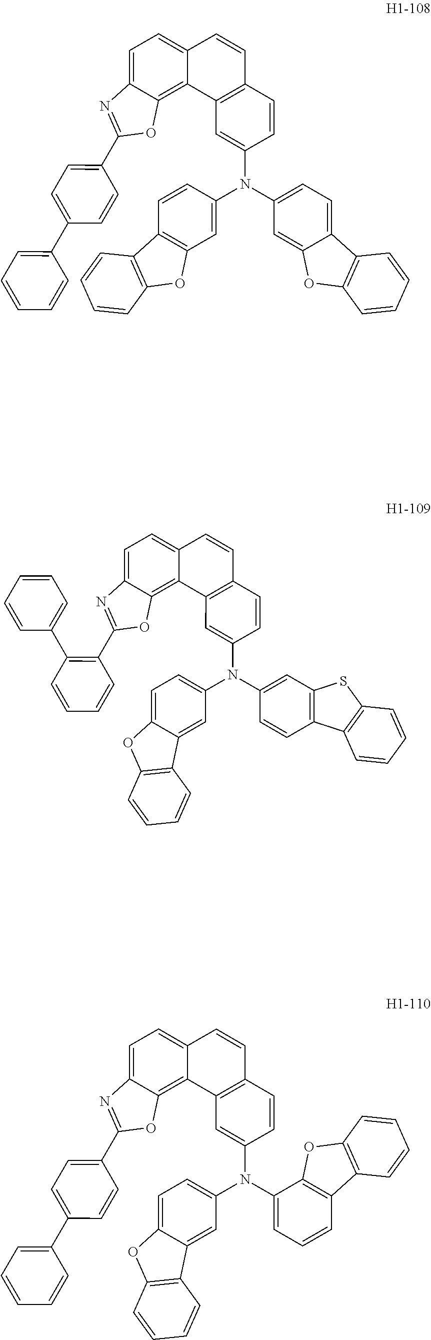 [SMILES: c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1 ; c1ccc(-c2ccc(-c3nc4ccc5ccc6ccc(N(c7ccc8oc9ccccc9c8c7)c7cccc8c7oc7ccccc78)cc6c5c4o3)cc2)cc1]

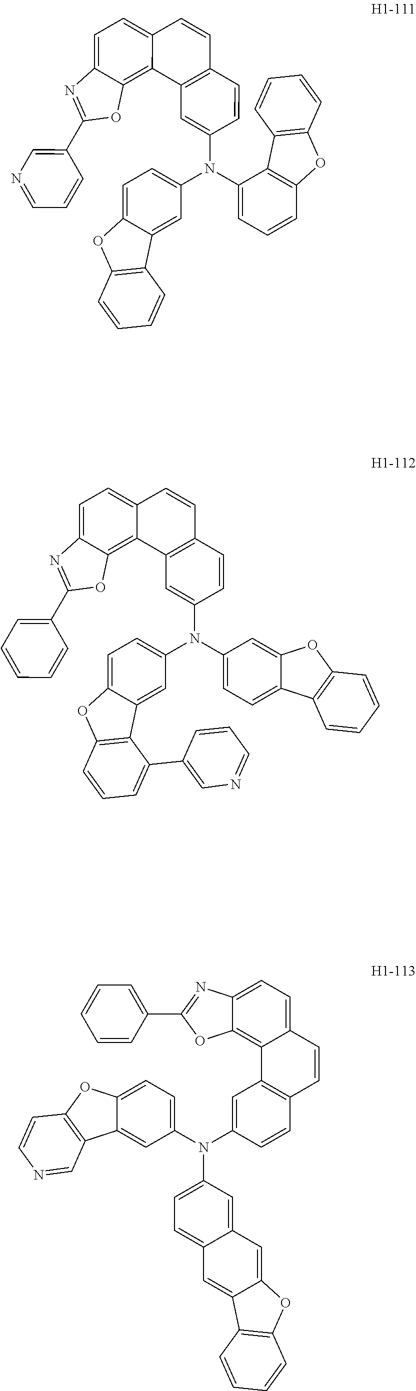 [SMILES: c1cncc(-c2nc3ccc4ccc5ccc(N(c6ccc7oc8ccccc8c7c6)c6cccc7oc8ccccc8c67)cc5c4c3o2)c1 ; c1ccc(-c2nc3ccc4ccc5ccc(N(c6ccc7c(c6)oc6ccccc67)c6ccc7oc8cccc(-c9cccnc9)c8c7c6)cc5c4c3o2)cc1 ; c1ccc(-c2nc3ccc4ccc5ccc(N(c6ccc7cc8c(cc7c6)oc6ccccc68)c6ccc7oc8ccncc8c7c6)cc5c4c3o2)cc1]

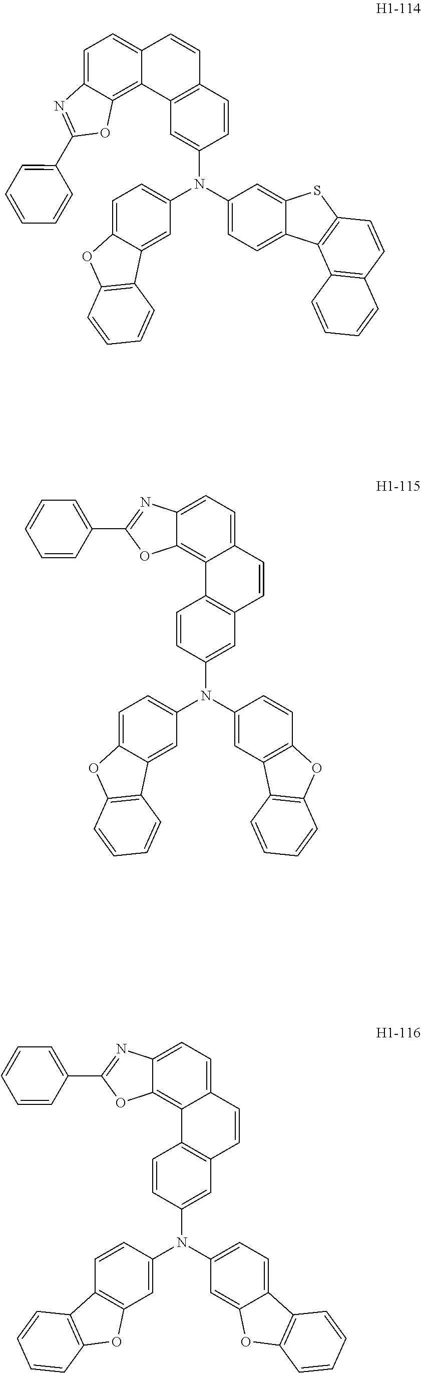 [SMILES: c1ccc(-c2nc3ccc4ccc5ccc(N(c6ccc7c(c6)sc6ccc8ccccc8c67)c6ccc7oc8ccccc8c7c6)cc5c4c3o2)cc1 ; c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1]

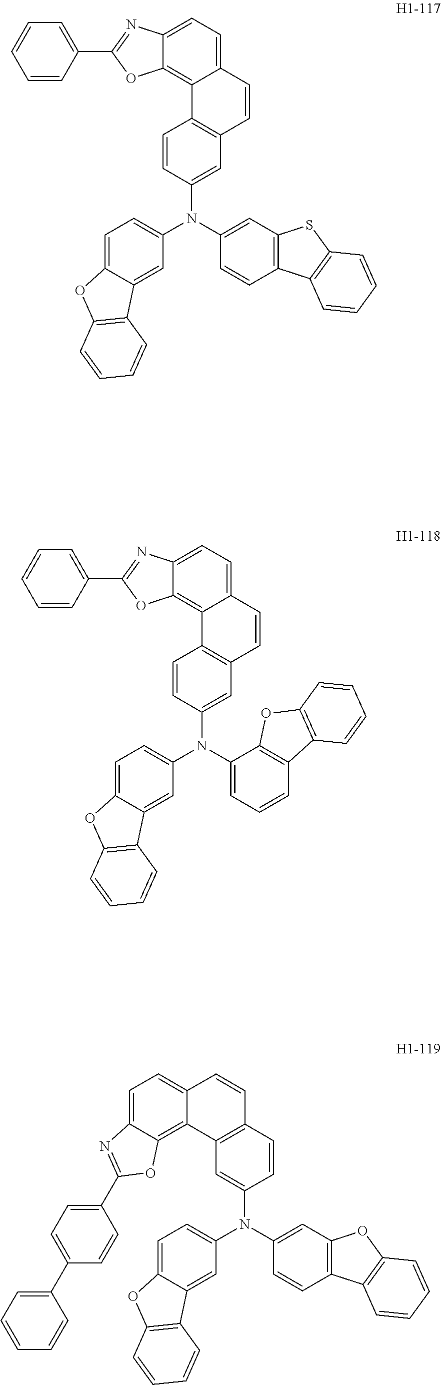 [SMILES: c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7oc8ccccc8c7c6)c6cccc7c6oc6ccccc67)ccc5c4c3o2)cc1 ; c1ccc(-c2ccc(-c3nc4ccc5ccc6ccc(N(c7ccc8c(c7)oc7ccccc78)c7ccc8oc9ccccc9c8c7)cc6c5c4o3)cc2)cc1]

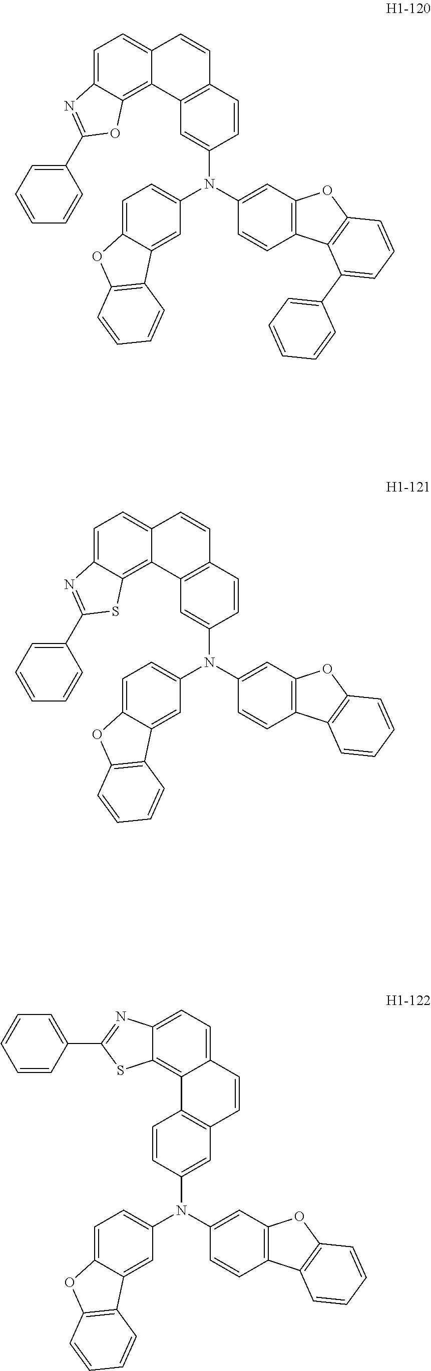 [SMILES: c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2nc3ccc4ccc5cc(N(c6ccc7c(c6)oc6ccccc67)c6ccc7oc8ccccc8c7c6)ccc5c4c3s2)cc1]

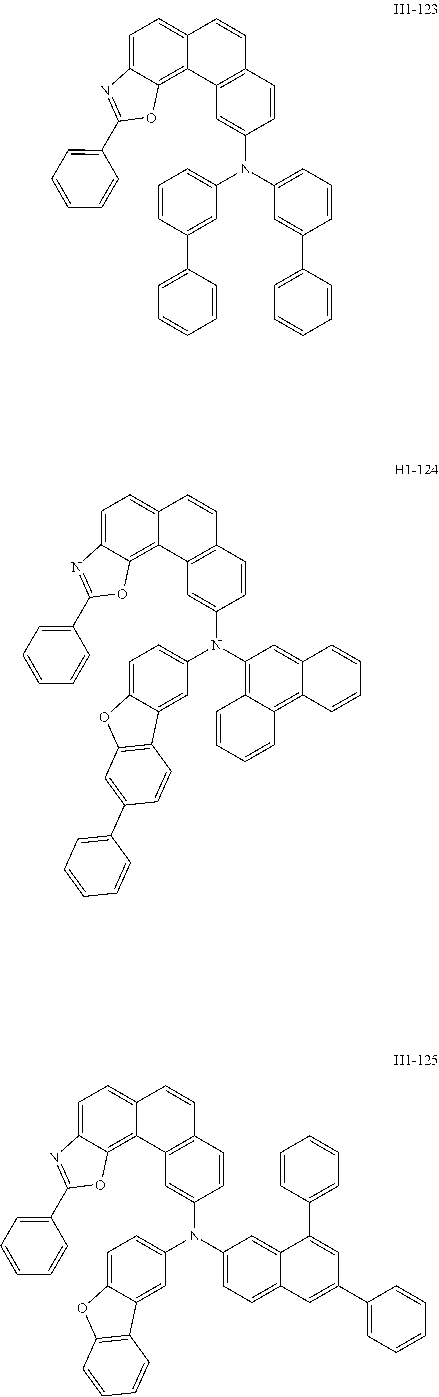 [SMILES: c1ccc(-c2cccc(N(c3cccc(-c4ccccc4)c3)c3ccc4ccc5ccc6nc(-c7ccccc7)oc6c5c4c3)c2)cc1 ; c1ccc(-c2ccc3c(c2)oc2ccc(N(c4ccc5ccc6ccc7nc(-c8ccccc8)oc7c6c5c4)c4cc5ccccc5c5ccccc45)cc23)cc1 ; c1ccc(-c2cc(-c3ccccc3)c3cc(N(c4ccc5oc6ccccc6c5c4)c4ccc5ccc6ccc7nc(-c8ccccc8)oc7c6c5c4)ccc3c2)cc1]

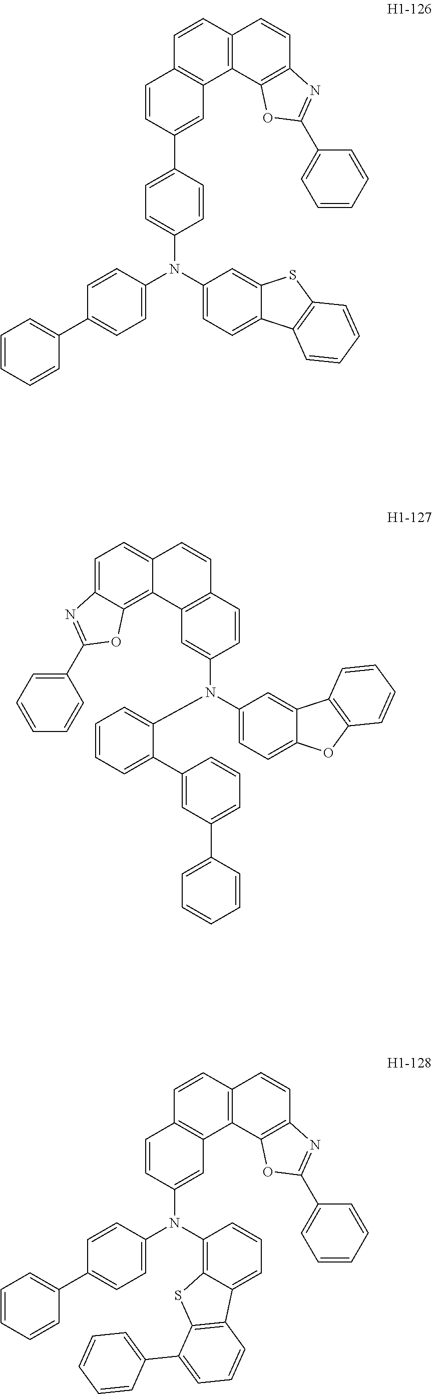

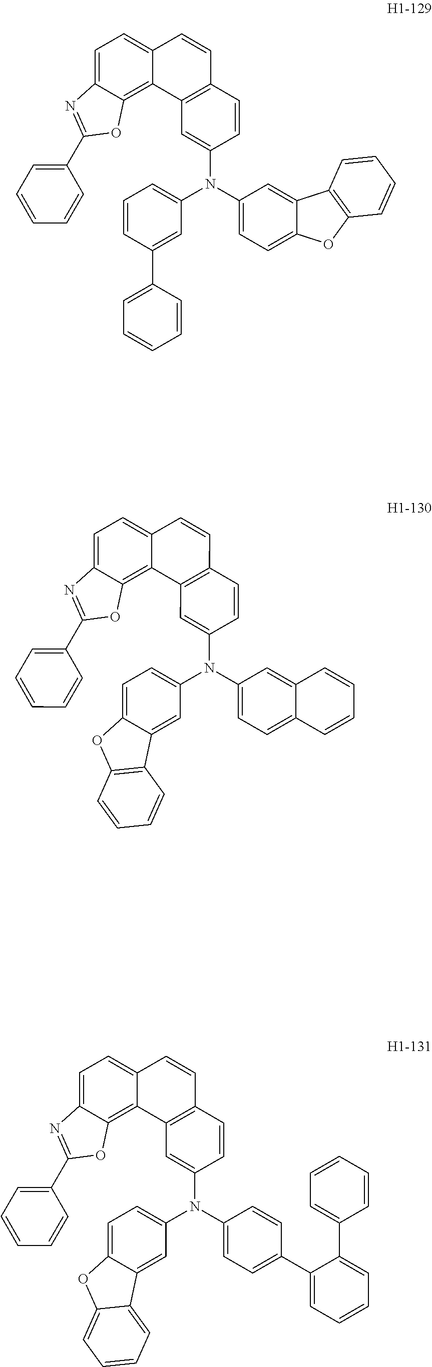 [SMILES: c1ccc(-c2cccc(N(c3ccc4oc5ccccc5c4c3)c3ccc4ccc5ccc6nc(-c7ccccc7)oc6c5c4c3)c2)cc1 ; c1ccc(-c2nc3ccc4ccc5ccc(N(c6ccc7ccccc7c6)c6ccc7oc8ccccc8c7c6)cc5c4c3o2)cc1 ; c1ccc(-c2nc3ccc4ccc5ccc(N(c6ccc(-c7ccccc7-c7ccccc7)cc6)c6ccc7oc8ccccc8c7c6)cc5c4c3o2)cc1]

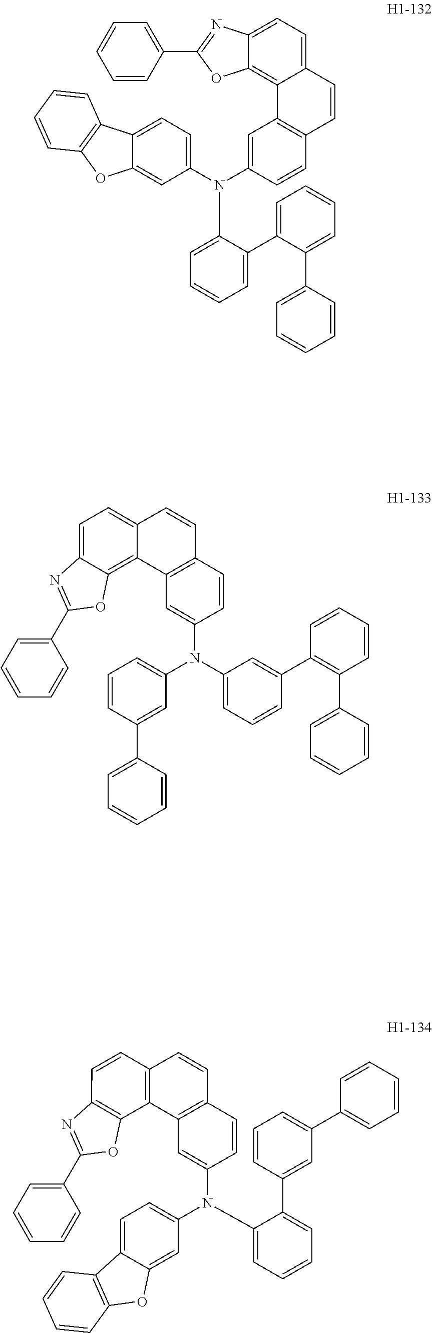 [SMILES: c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1 ; c1ccc(-c2cccc(-c3ccccc3N(c3ccc4c(c3)oc3ccccc34)c3ccc4ccc5ccc6nc(-c7ccccc7)oc6c5c4c3)c2)cc1]

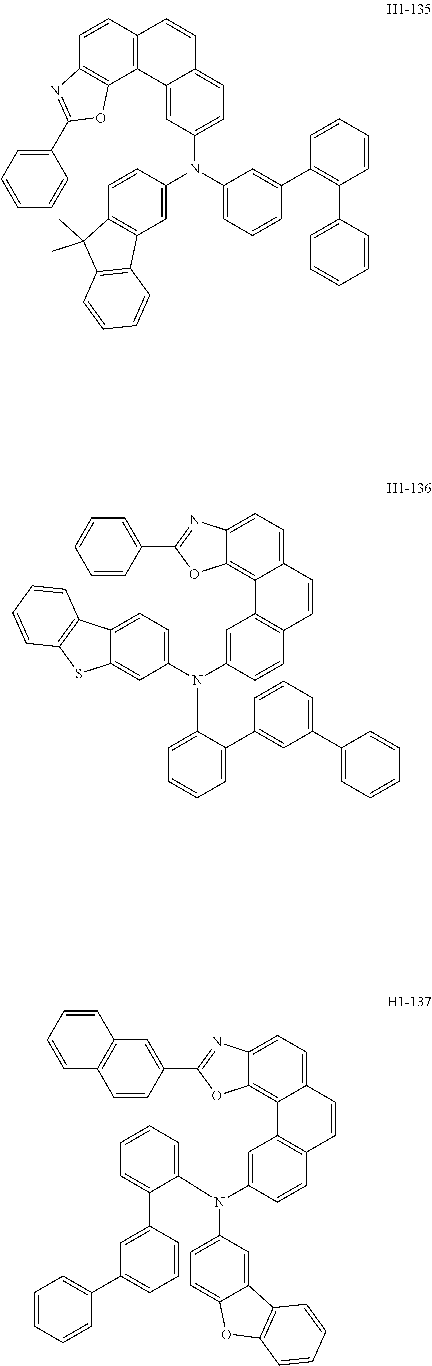 [SMILES: CC1(C)c2ccccc2-c2cc(N(c3cccc(-c4ccccc4-c4ccccc4)c3)c3ccc4ccc5ccc6nc(-c7ccccc7)oc6c5c4c3)ccc21 ; c1ccc(-c2cccc(-c3ccccc3N(c3ccc4c(c3)sc3ccccc34)c3ccc4ccc5ccc6nc(-c7ccccc7)oc6c5c4c3)c2)cc1 ; c1ccc(-c2cccc(-c3ccccc3N(c3ccc4oc5ccccc5c4c3)c3ccc4ccc5ccc6nc(-c7ccc8ccccc8c7)oc6c5c4c3)c2)cc1]

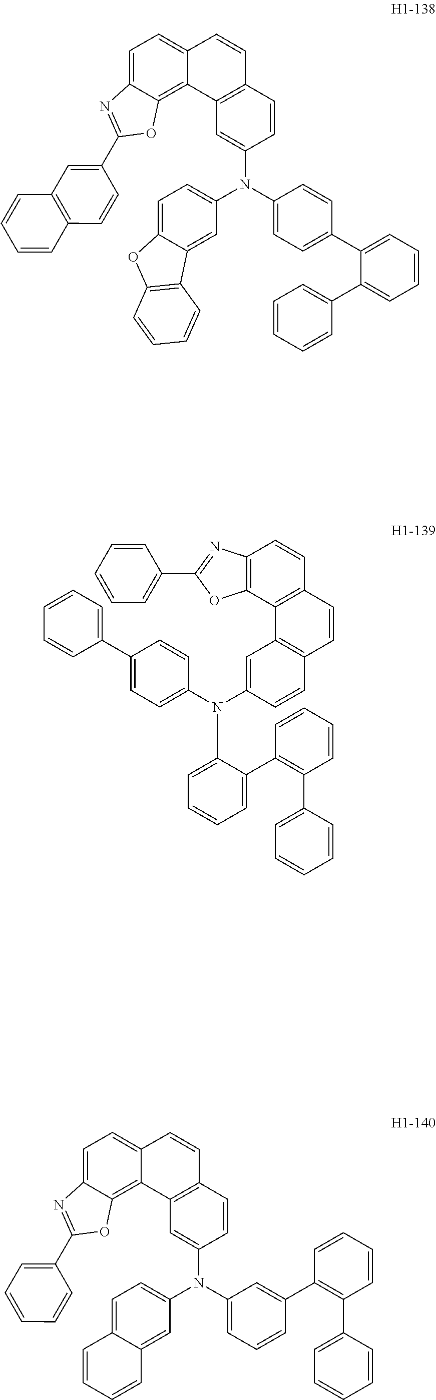 [SMILES: c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1 ; c1ccc(-c2nc3ccc4ccc5ccc(N(c6cccc(-c7ccccc7-c7ccccc7)c6)c6ccc7ccccc7c6)cc5c4c3o2)cc1]

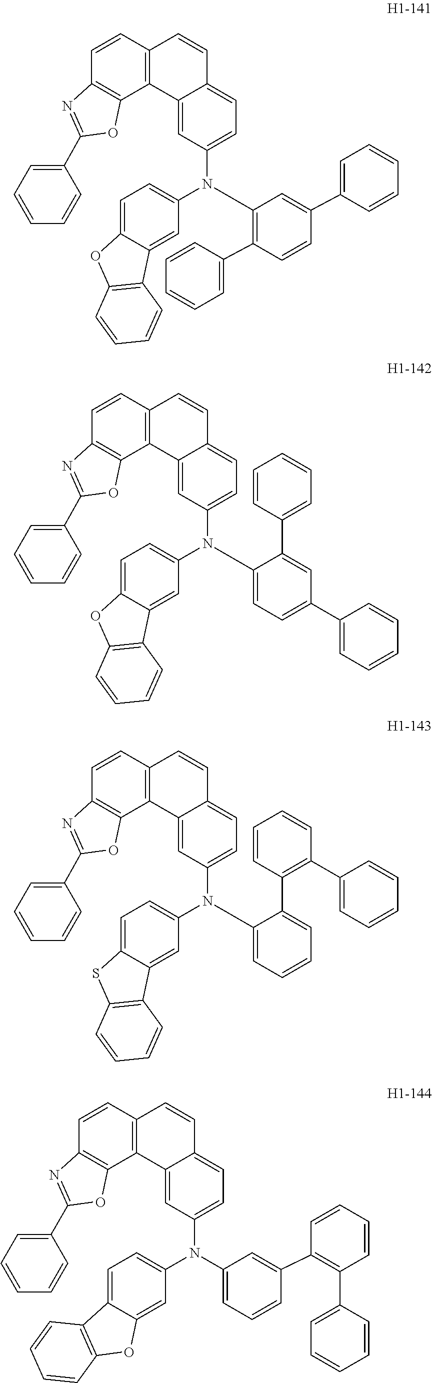

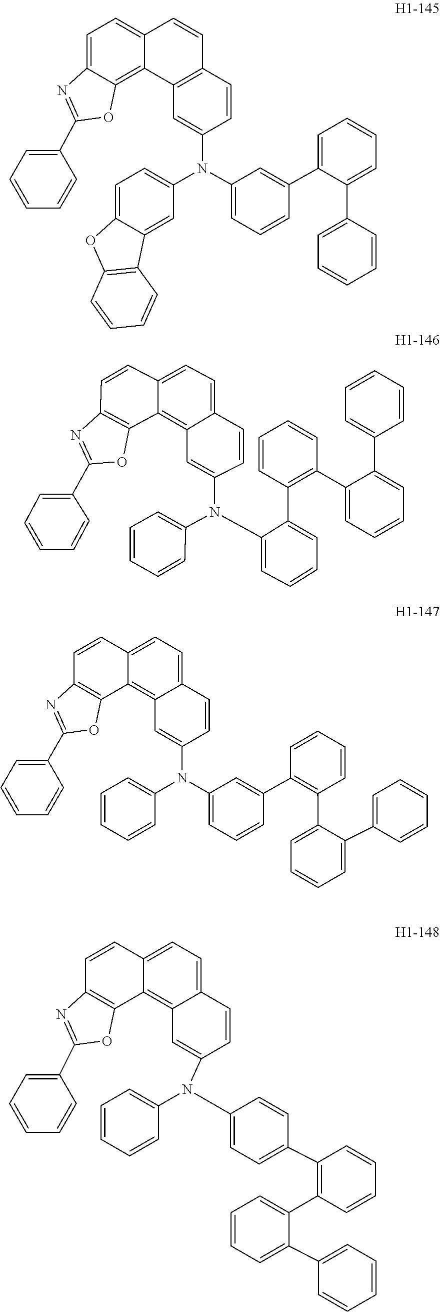

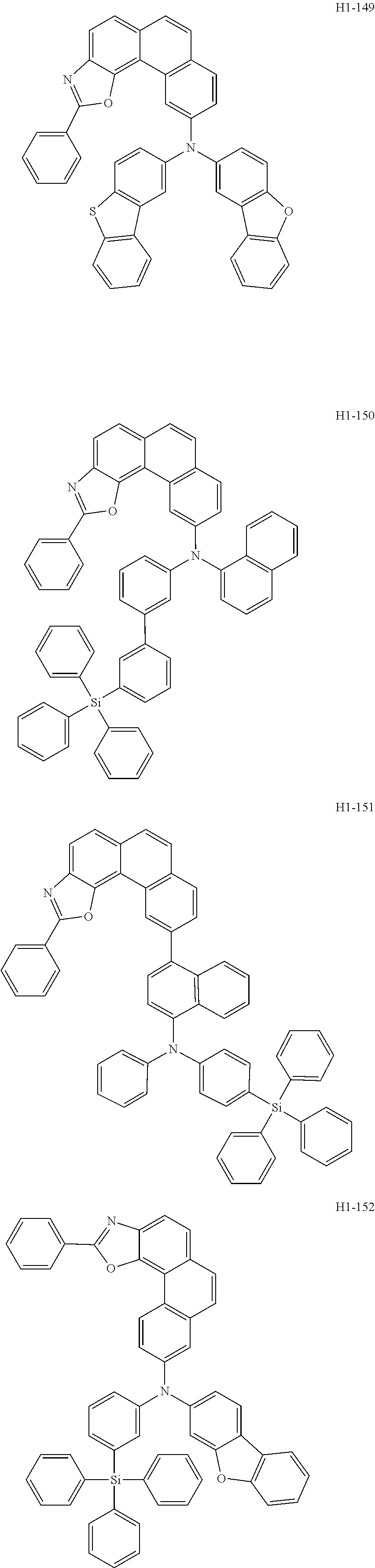 [SMILES: c1ccc(-c2nc3ccc4ccc5ccc(N(c6ccc7oc8ccccc8c7c6)c6ccc7sc8ccccc8c7c6)cc5c4c3o2)cc1 ; c1ccc(-c2nc3ccc4ccc5ccc(N(c6cccc(-c7cccc([Si](c8ccccc8)(c8ccccc8)c8ccccc8)c7)c6)c6cccc7ccccc67)cc5c4c3o2)cc1 ; c1ccc(-c2nc3ccc4ccc5ccc(-c6ccc(N(c7ccccc7)c7ccc([Si](c8ccccc8)(c8ccccc8)c8ccccc8)cc7)c7ccccc67)cc5c4c3o2)cc1 ; c1ccc(-c2nc3ccc4ccc5cc(N(c6cccc([Si](c7ccccc7)(c7ccccc7)c7ccccc7)c6)c6ccc7c(c6)oc6ccccc67)ccc5c4c3o2)cc1]

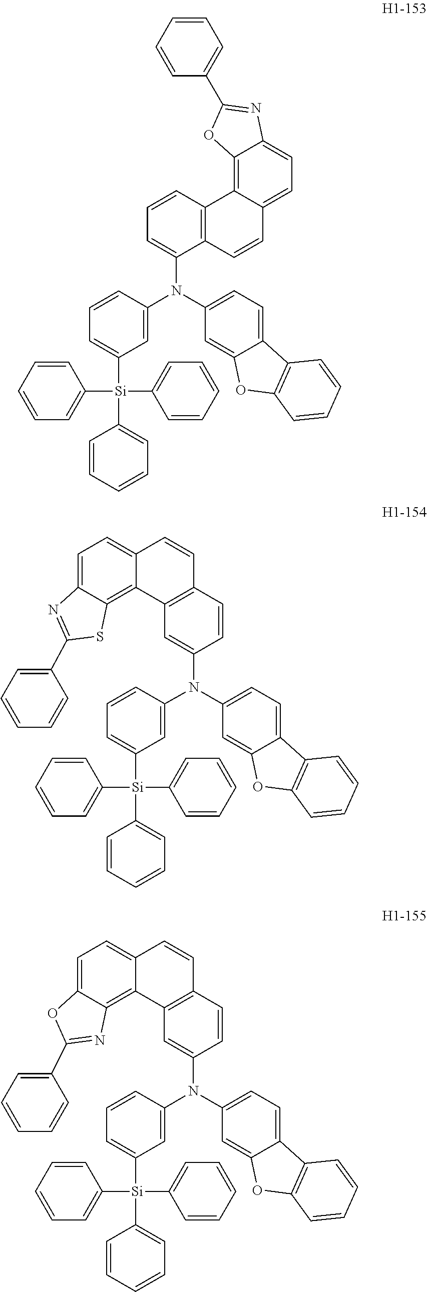 [SMILES: c1ccc(-c2nc3ccc4ccc5c(N(c6cccc([Si](c7ccccc7)(c7ccccc7)c7ccccc7)c6)c6ccc7c(c6)oc6ccccc67)cccc5c4c3o2)cc1 ; c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3s2)cc1 ; c1ccc(-c2nc3c(ccc4ccc5ccc(N(c6cccc([Si](c7ccccc7)(c7ccccc7)c7ccccc7)c6)c6ccc7c(c6)oc6ccccc67)cc5c43)o2)cc1]

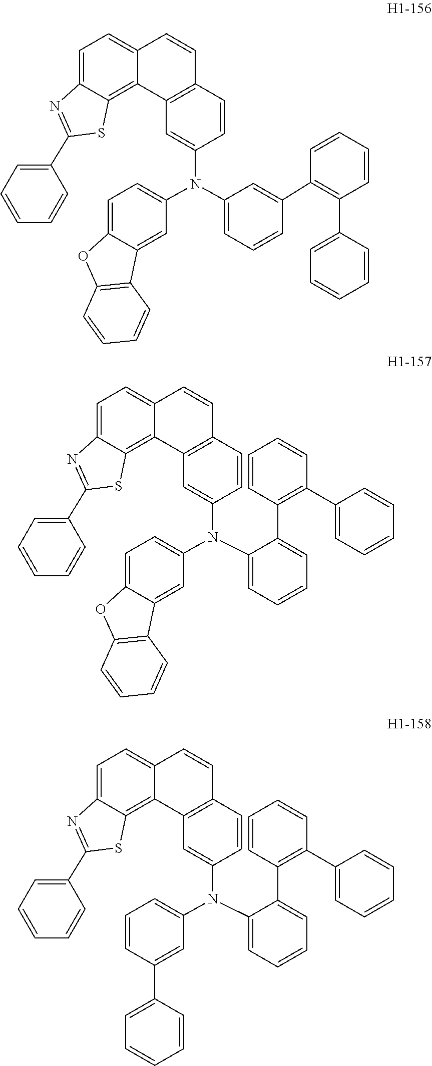 [SMILES: c1ccc(-c2nc3ccc4ccc5ccc(N(c6cccc(-c7ccccc7-c7ccccc7)c6)c6ccc7oc8ccccc8c7c6)cc5c4c3s2)cc1 ; c1ccc(-c2nc3ccc4ccc5ccc(N(c6ccc7oc8ccccc8c7c6)c6ccccc6-c6ccccc6-c6ccccc6)cc5c4c3s2)cc1 ; c1ccc(-c2cccc(N(c3ccc4ccc5ccc6nc(-c7ccccc7)sc6c5c4c3)c3ccccc3-c3ccccc3-c3ccccc3)c2)cc1]

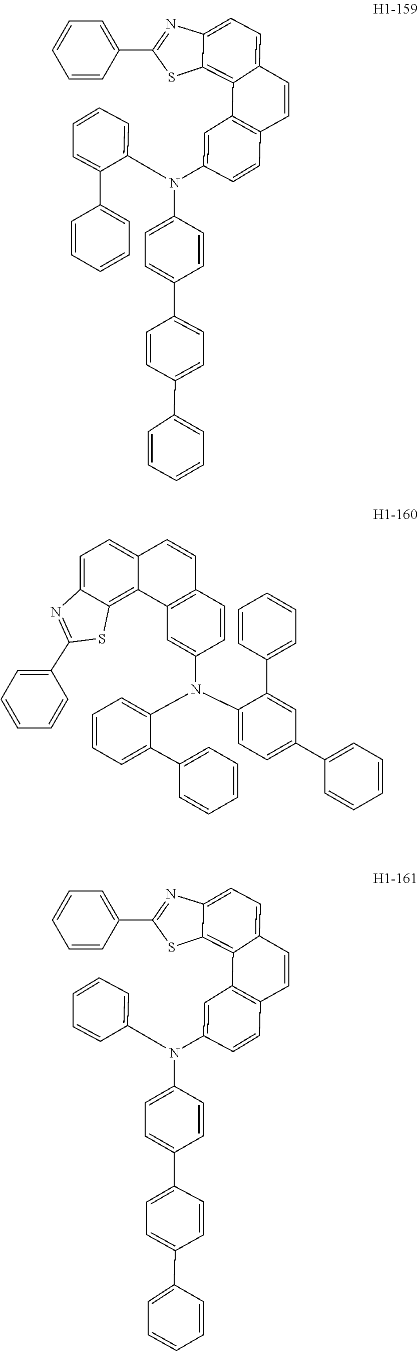 [SMILES: c1ccc(-c2ccc(-c3ccc(N(c4ccc5ccc6ccc7nc(-c8ccccc8)sc7c6c5c4)c4ccccc4-c4ccccc4)cc3)cc2)cc1 ; c1ccc(-c2ccc(N(c3ccc4ccc5ccc6nc(-c7ccccc7)sc6c5c4c3)c3ccccc3-c3ccccc3)c(-c3ccccc3)c2)cc1 ; c1ccc(-c2ccc(-c3ccc(N(c4ccccc4)c4ccc5ccc6ccc7nc(-c8ccccc8)sc7c6c5c4)cc3)cc2)cc1]

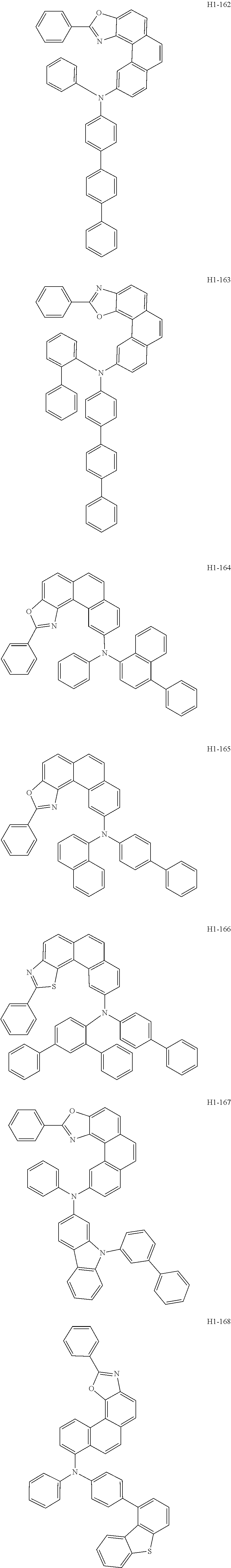

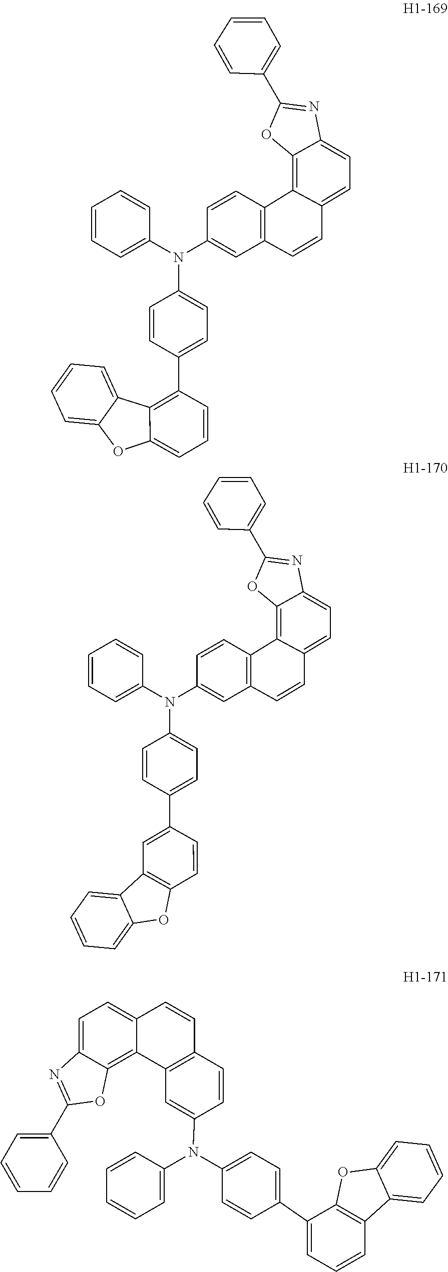

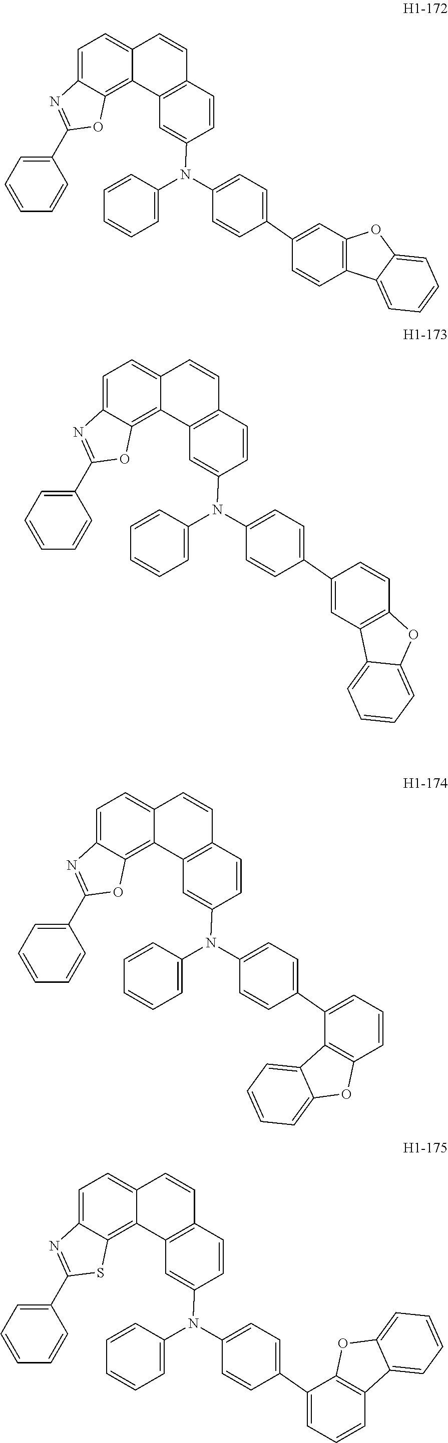 [SMILES: c1ccc(-c2nc3ccc4ccc5ccc(N(c6ccccc6)c6ccc(-c7ccc8c(c7)oc7ccccc78)cc6)cc5c4c3o2)cc1 ; c1ccc(-c2nc3ccc4ccc5ccc(N(c6ccccc6)c6ccc(-c7ccc8oc9ccccc9c8c7)cc6)cc5c4c3o2)cc1 ; c1ccc(-c2nc3ccc4ccc5ccc(N(c6ccccc6)c6ccc(-c7cccc8oc9ccccc9c78)cc6)cc5c4c3o2)cc1 ; c1ccc(-c2nc3ccc4ccc5ccc(N(c6ccccc6)c6ccc(-c7cccc8c7oc7ccccc78)cc6)cc5c4c3s2)cc1]

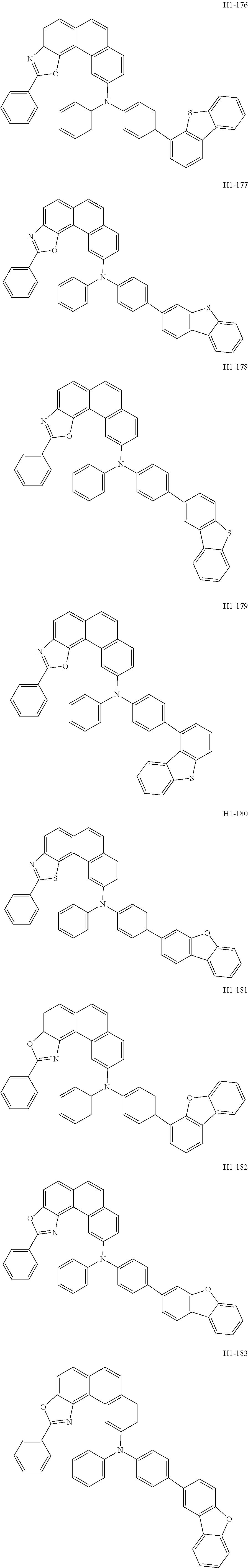

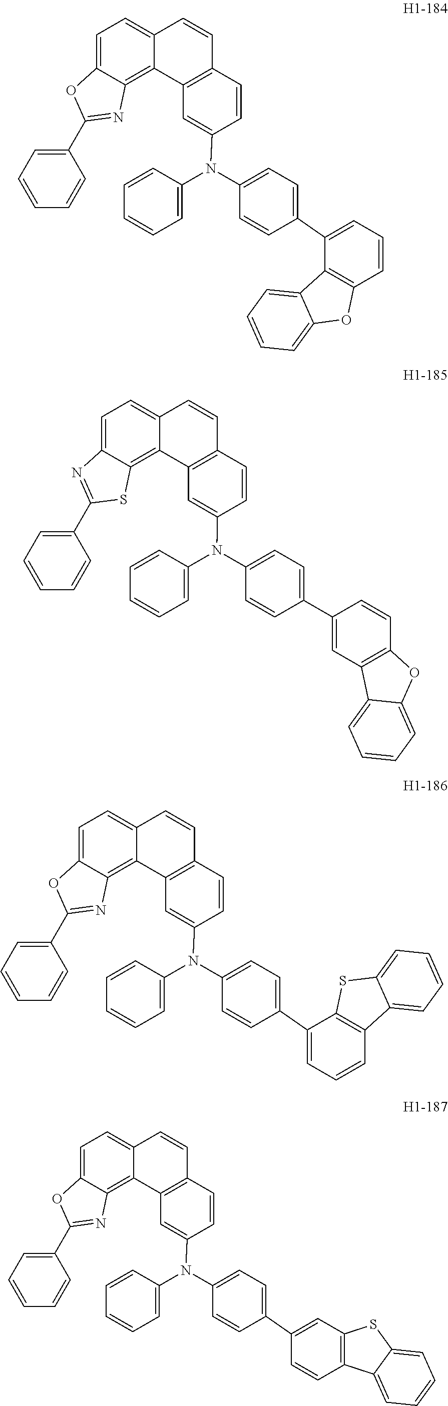 [SMILES: c1ccc(-c2nc3c(ccc4ccc5ccc(N(c6ccccc6)c6ccc(-c7cccc8oc9ccccc9c78)cc6)cc5c43)o2)cc1 ; c1ccc(-c2nc3ccc4ccc5ccc(N(c6ccccc6)c6ccc(-c7ccc8oc9ccccc9c8c7)cc6)cc5c4c3s2)cc1 ; c1ccc(-c2nc3c(ccc4ccc5ccc(N(c6ccccc6)c6ccc(-c7cccc8c7sc7ccccc78)cc6)cc5c43)o2)cc1 ; c1ccc(-c2nc3c(ccc4ccc5ccc(N(c6ccccc6)c6ccc(-c7ccc8c(c7)sc7ccccc78)cc6)cc5c43)o2)cc1]

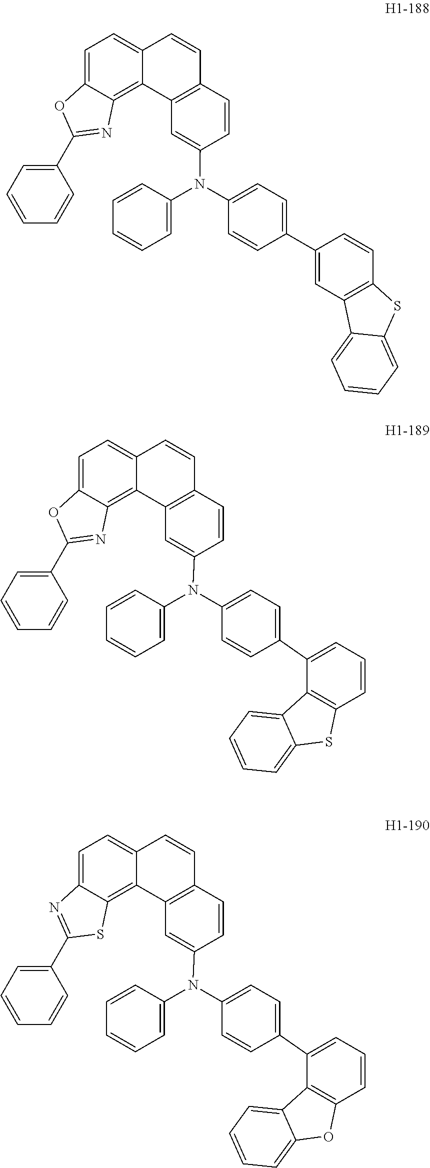 [SMILES: c1ccc(-c2nc3c(ccc4ccc5ccc(N(c6ccccc6)c6ccc(-c7ccc8sc9ccccc9c8c7)cc6)cc5c43)o2)cc1 ; c1ccc(-c2nc3c(ccc4ccc5ccc(N(c6ccccc6)c6ccc(-c7cccc8sc9ccccc9c78)cc6)cc5c43)o2)cc1 ; c1ccc(-c2nc3ccc4ccc5ccc(N(c6ccccc6)c6ccc(-c7cccc8oc9ccccc9c78)cc6)cc5c4c3s2)cc1]

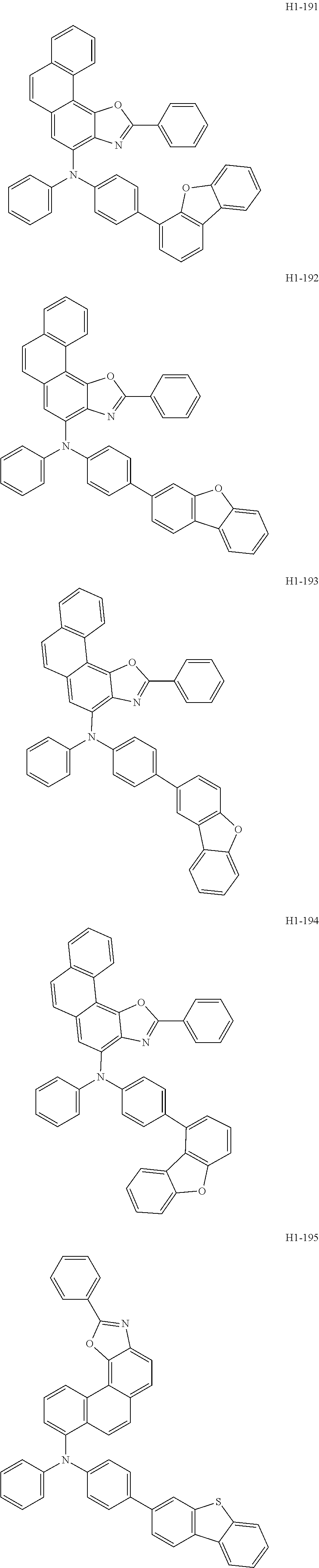 [SMILES: c1ccc(-c2nc3c(N(c4ccccc4)c4ccc(-c5cccc6c5oc5ccccc56)cc4)cc4ccc5ccccc5c4c3o2)cc1 ; c1ccc(-c2nc3c(N(c4ccccc4)c4ccc(-c5ccc6c(c5)oc5ccccc56)cc4)cc4ccc5ccccc5c4c3o2)cc1 ; c1ccc(-c2nc3c(N(c4ccccc4)c4ccc(-c5ccc6oc7ccccc7c6c5)cc4)cc4ccc5ccccc5c4c3o2)cc1 ; c1ccc(-c2nc3c(N(c4ccccc4)c4ccc(-c5cccc6oc7ccccc7c56)cc4)cc4ccc5ccccc5c4c3o2)cc1 ; c1ccc(-c2nc3ccc4ccc5c(N(c6ccccc6)c6ccc(-c7ccc8c(c7)sc7ccccc78)cc6)cccc5c4c3o2)cc1]

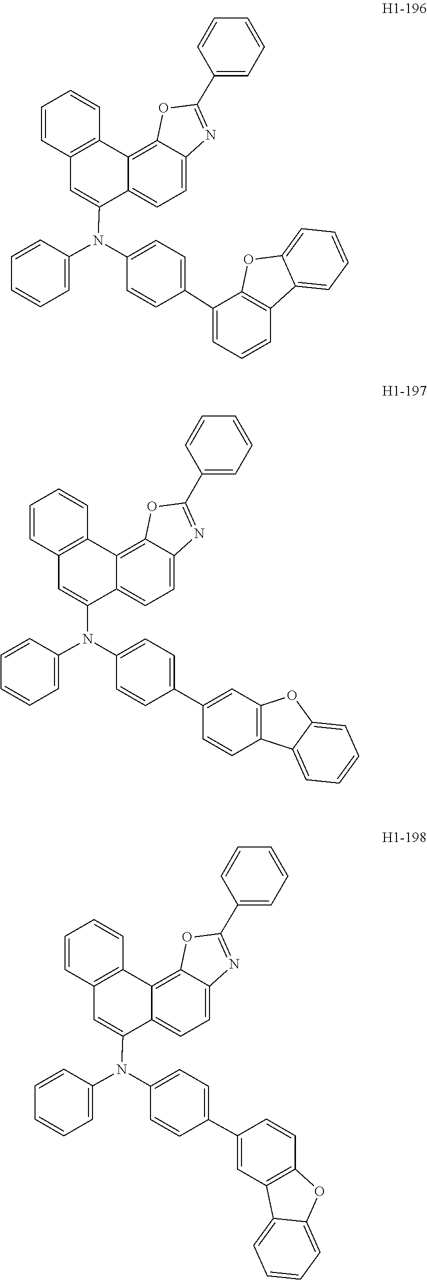 [SMILES: c1ccc(-c2nc3ccc4c(N(c5ccccc5)c5ccc(-c6cccc7c6oc6ccccc67)cc5)cc5ccccc5c4c3o2)cc1 ; c1ccc(-c2nc3ccc4c(N(c5ccccc5)c5ccc(-c6ccc7c(c6)oc6ccccc67)cc5)cc5ccccc5c4c3o2)cc1 ; c1ccc(-c2nc3ccc4c(N(c5ccccc5)c5ccc(-c6ccc7oc8ccccc8c7c6)cc5)cc5ccccc5c4c3o2)cc1]

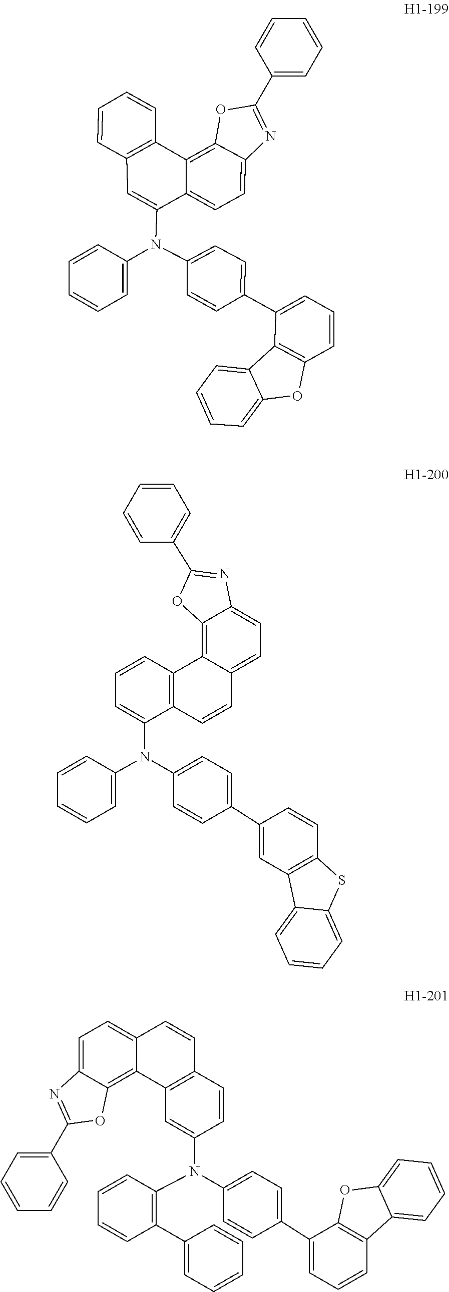 [SMILES: c1ccc(-c2nc3ccc4c(N(c5ccccc5)c5ccc(-c6cccc7oc8ccccc8c67)cc5)cc5ccccc5c4c3o2)cc1 ; c1ccc(-c2nc3ccc4ccc5c(N(c6ccccc6)c6ccc(-c7ccc8sc9ccccc9c8c7)cc6)cccc5c4c3o2)cc1 ; c1ccc(-c2nc3ccc4ccc5ccc(N(c6ccc(-c7cccc8c7oc7ccccc78)cc6)c6ccccc6-c6ccccc6)cc5c4c3o2)cc1]

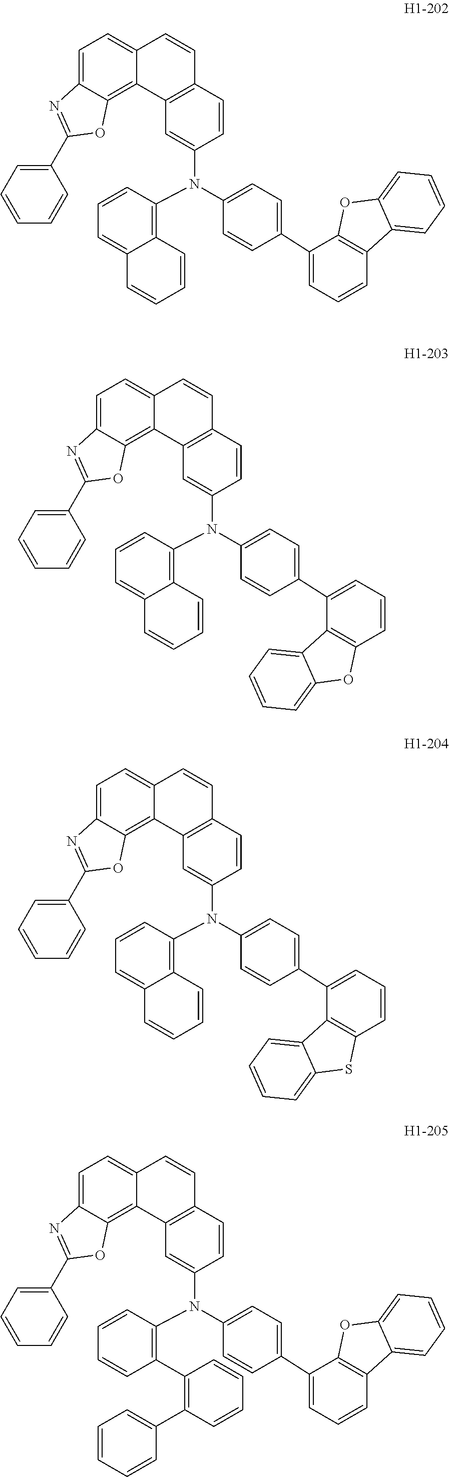 [SMILES: c1ccc(-c2nc3ccc4ccc5ccc(N(c6ccc(-c7cccc8c7oc7ccccc78)cc6)c6cccc7ccccc67)cc5c4c3o2)cc1 ; c1ccc(-c2nc3ccc4ccc5ccc(N(c6ccc(-c7cccc8oc9ccccc9c78)cc6)c6cccc7ccccc67)cc5c4c3o2)cc1 ; c1ccc(-c2nc3ccc4ccc5ccc(N(c6ccc(-c7cccc8sc9ccccc9c78)cc6)c6cccc7ccccc67)cc5c4c3o2)cc1 ; c1ccc(-c2nc3ccc4ccc5ccc(N(c6ccc(-c7cccc8c7oc7ccccc78)cc6)c6ccccc6-c6ccccc6-c6ccccc6)cc5c4c3o2)cc1]

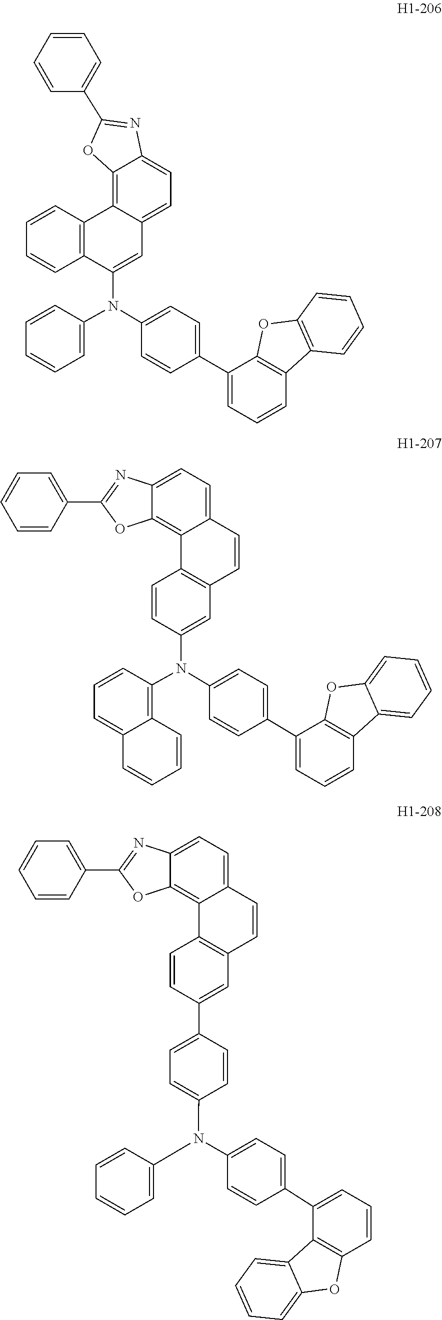 [SMILES: c1ccc(-c2nc3ccc4cc(N(c5ccccc5)c5ccc(-c6cccc7c6oc6ccccc67)cc5)c5ccccc5c4c3o2)cc1 ; c1ccc(-c2nc3ccc4ccc5cc(N(c6ccc(-c7cccc8c7oc7ccccc78)cc6)c6cccc7ccccc67)ccc5c4c3o2)cc1 ; c1ccc(-c2nc3ccc4ccc5cc(-c6ccc(N(c7ccccc7)c7ccc(-c8cccc9oc%10ccccc%10c89)cc7)cc6)ccc5c4c3o2)cc1]

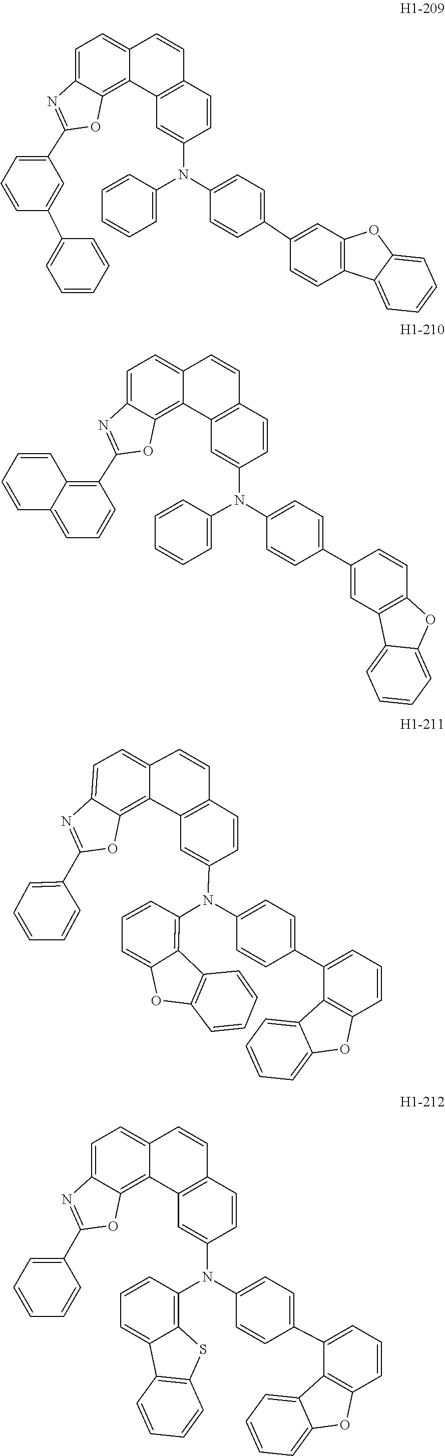 [SMILES: c1ccc(-c2cccc(-c3nc4ccc5ccc6ccc(N(c7ccccc7)c7ccc(-c8ccc9c(c8)oc8ccccc89)cc7)cc6c5c4o3)c2)cc1 ; c1ccc(N(c2ccc(-c3ccc4oc5ccccc5c4c3)cc2)c2ccc3ccc4ccc5nc(-c6cccc7ccccc67)oc5c4c3c2)cc1 ; c1ccc(-c2nc3ccc4ccc5ccc(N(c6ccc(-c7cccc8oc9ccccc9c78)cc6)c6cccc7oc8ccccc8c67)cc5c4c3o2)cc1 ; c1ccc(-c2nc3ccc4ccc5ccc(N(c6ccc(-c7cccc8oc9ccccc9c78)cc6)c6cccc7c6sc6ccccc67)cc5c4c3o2)cc1]

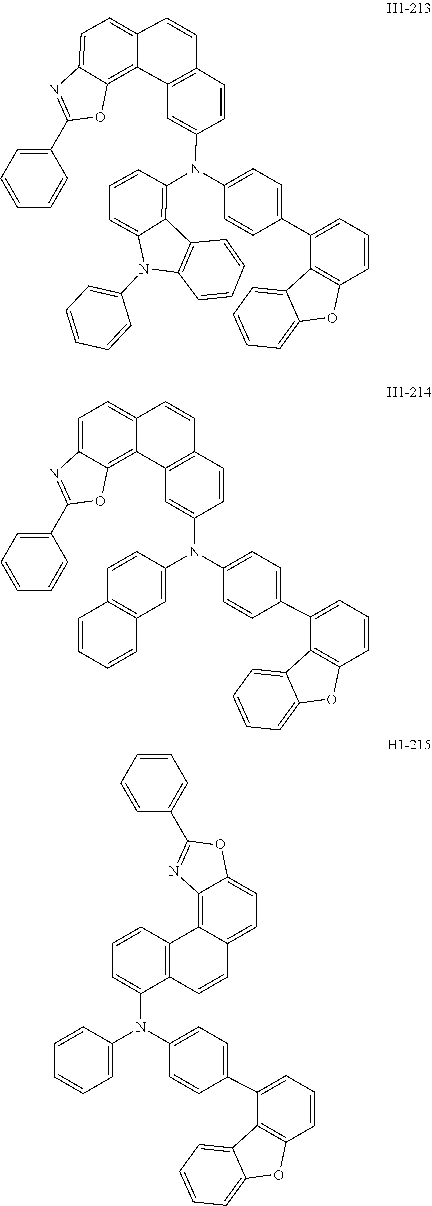 [SMILES: c1ccc(-c2nc3ccc4ccc5ccc(N(c6ccc(-c7cccc8oc9ccccc9c78)cc6)c6cccc7c6c6ccccc6n7-c6ccccc6)cc5c4c3o2)cc1 ; c1ccc(-c2nc3ccc4ccc5ccc(N(c6ccc(-c7cccc8oc9ccccc9c78)cc6)c6ccc7ccccc7c6)cc5c4c3o2)cc1 ; c1ccc(-c2nc3c(ccc4ccc5c(N(c6ccccc6)c6ccc(-c7cccc8oc9ccccc9c78)cc6)cccc5c43)o2)cc1]

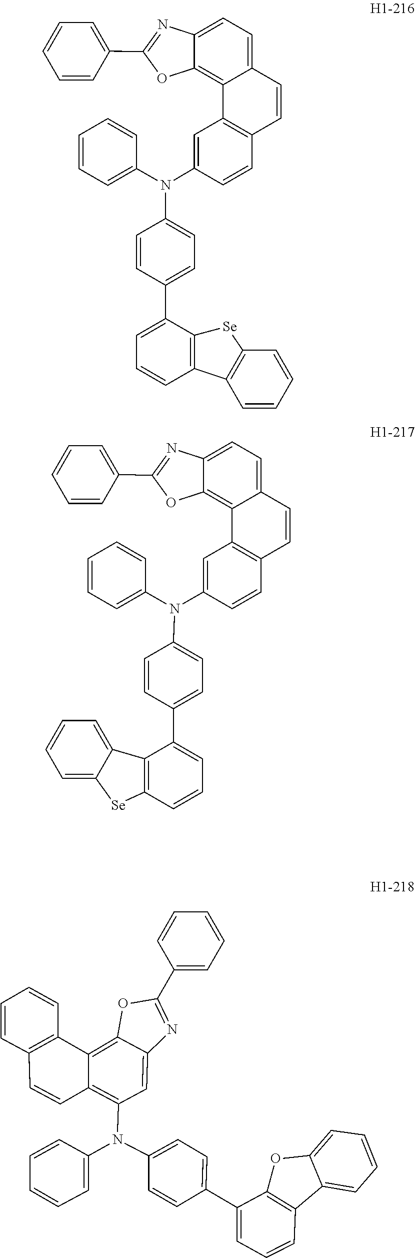

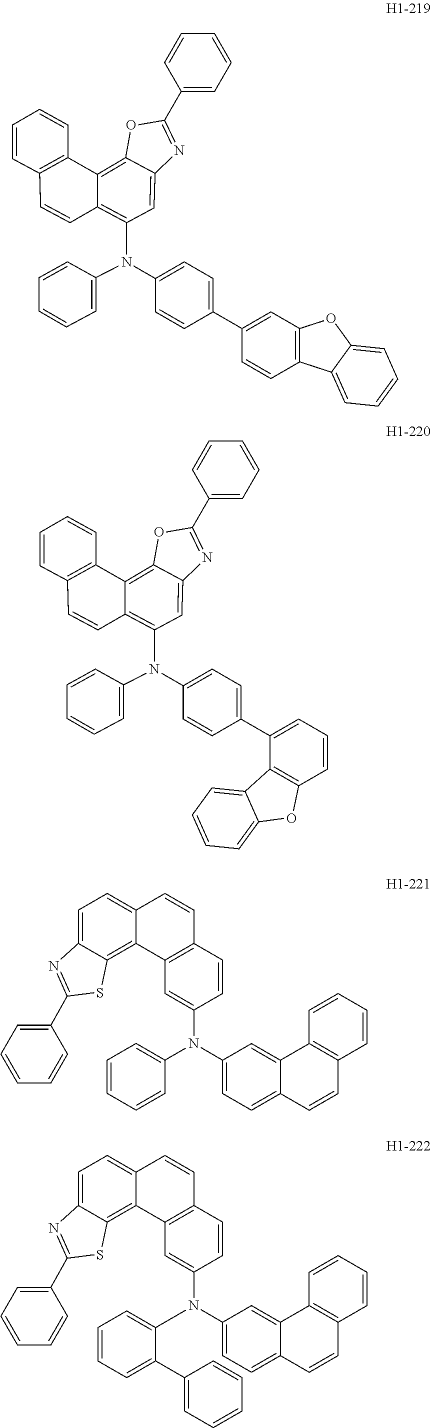 [SMILES: c1ccc(-c2nc3cc(N(c4ccccc4)c4ccc(-c5ccc6c(c5)oc5ccccc56)cc4)c4ccc5ccccc5c4c3o2)cc1 ; c1ccc(-c2nc3cc(N(c4ccccc4)c4ccc(-c5cccc6oc7ccccc7c56)cc4)c4ccc5ccccc5c4c3o2)cc1 ; c1ccc(-c2nc3ccc4ccc5ccc(N(c6ccccc6)c6ccc7ccc8ccccc8c7c6)cc5c4c3s2)cc1 ; c1ccc(-c2nc3ccc4ccc5ccc(N(c6ccc7ccc8ccccc8c7c6)c6ccccc6-c6ccccc6)cc5c4c3s2)cc1]

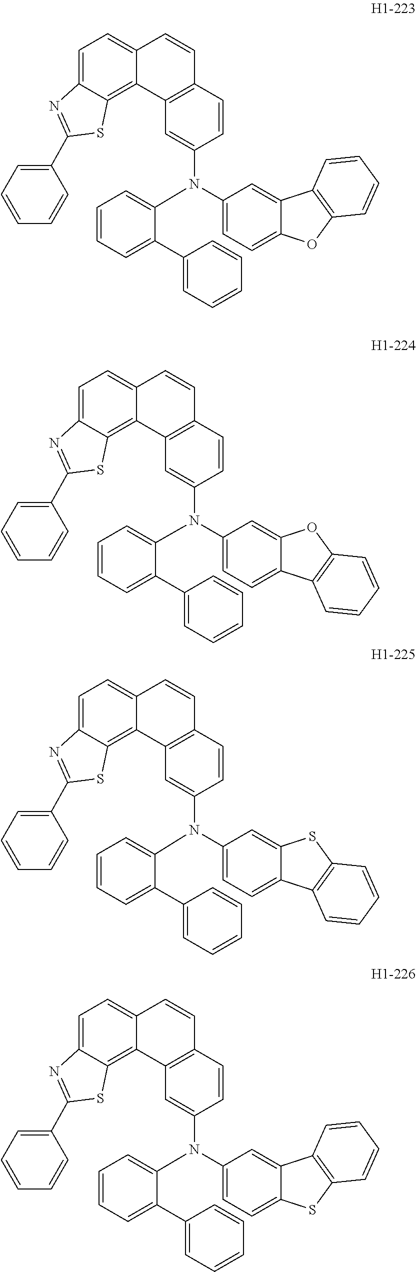 [SMILES: c1ccc(-c2nc3ccc4ccc5ccc(N(c6ccc7oc8ccccc8c7c6)c6ccccc6-c6ccccc6)cc5c4c3s2)cc1 ; c1ccc(-c2nc3ccc4ccc5ccc(N(c6ccc7c(c6)oc6ccccc67)c6ccccc6-c6ccccc6)cc5c4c3s2)cc1 ; c1ccc(-c2nc3ccc4ccc5ccc(N(c6ccc7c(c6)sc6ccccc67)c6ccccc6-c6ccccc6)cc5c4c3s2)cc1 ; c1ccc(-c2nc3ccc4ccc5ccc(N(c6ccc7sc8ccccc8c7c6)c6ccccc6-c6ccccc6)cc5c4c3s2)cc1]

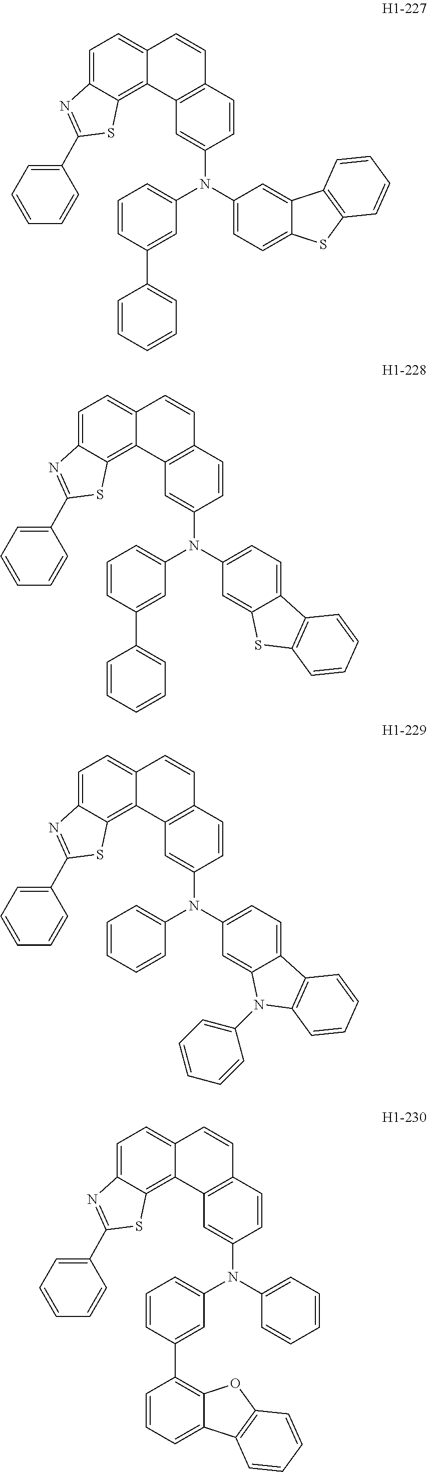

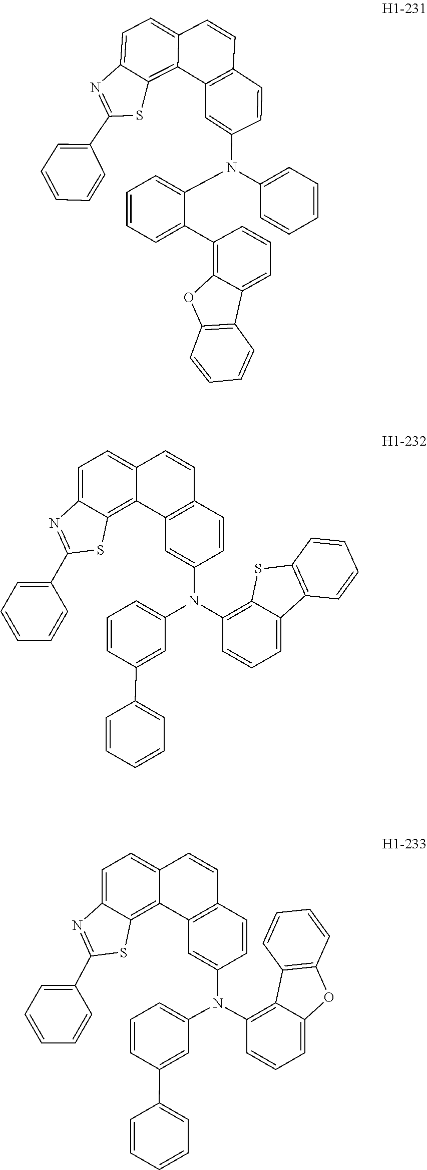 [SMILES: c1ccc(-c2nc3ccc4ccc5ccc(N(c6ccccc6)c6ccccc6-c6cccc7c6oc6ccccc67)cc5c4c3s2)cc1 ; c1ccc(-c2cccc(N(c3ccc4ccc5ccc6nc(-c7ccccc7)sc6c5c4c3)c3cccc4c3sc3ccccc34)c2)cc1 ; c1ccc(-c2cccc(N(c3ccc4ccc5ccc6nc(-c7ccccc7)sc6c5c4c3)c3cccc4oc5ccccc5c34)c2)cc1]

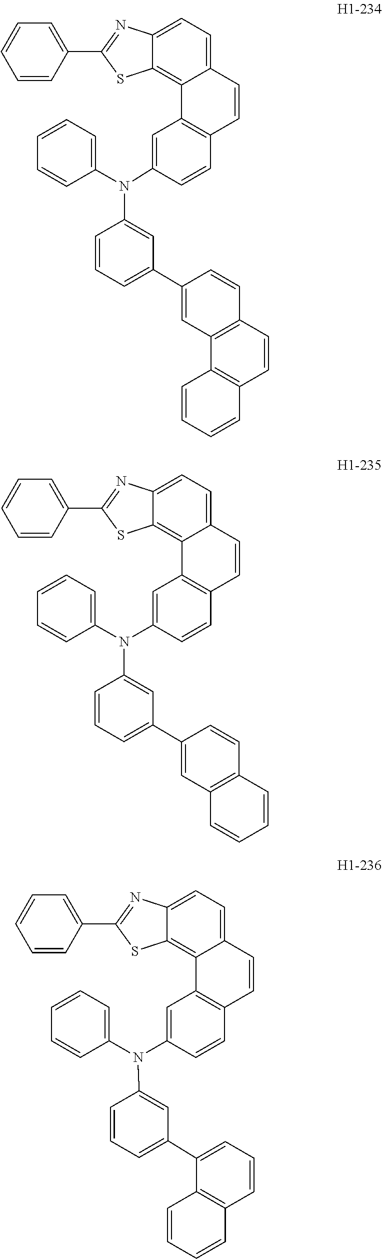 [SMILES: c1ccc(-c2nc3ccc4ccc5ccc(N(c6ccccc6)c6cccc(-c7ccc8ccc9ccccc9c8c7)c6)cc5c4c3s2)cc1 ; c1ccc(-c2nc3ccc4ccc5ccc(N(c6ccccc6)c6cccc(-c7ccc8ccccc8c7)c6)cc5c4c3s2)cc1 ; c1ccc(-c2nc3ccc4ccc5ccc(N(c6ccccc6)c6cccc(-c7cccc8ccccc78)c6)cc5c4c3s2)cc1]

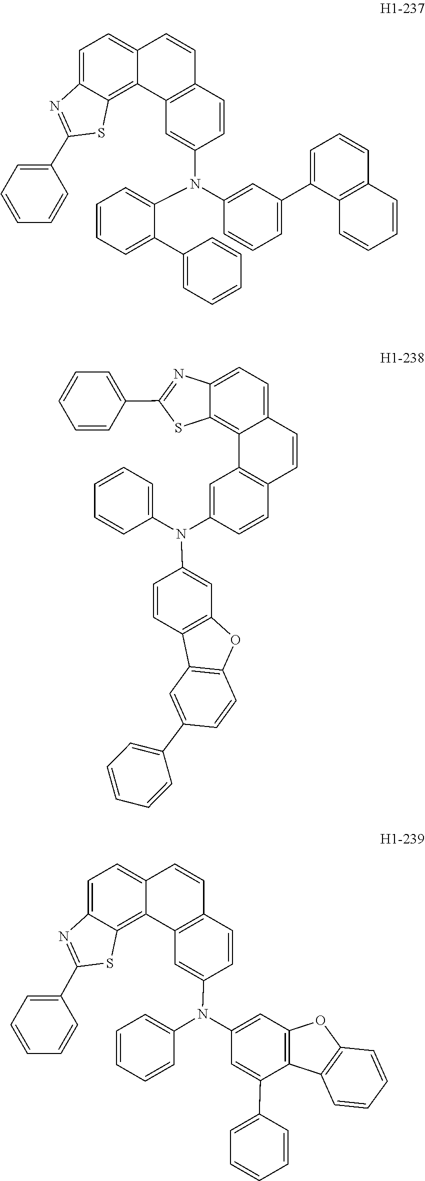 [SMILES: c1ccc(-c2nc3ccc4ccc5ccc(N(c6cccc(-c7cccc8ccccc78)c6)c6ccccc6-c6ccccc6)cc5c4c3s2)cc1 ; c1ccc(-c2ccc3oc4cc(N(c5ccccc5)c5ccc6ccc7ccc8nc(-c9ccccc9)sc8c7c6c5)ccc4c3c2)cc1 ; c1ccc(-c2nc3ccc4ccc5ccc(N(c6ccccc6)c6cc(-c7ccccc7)c7c(c6)oc6ccccc67)cc5c4c3s2)cc1]

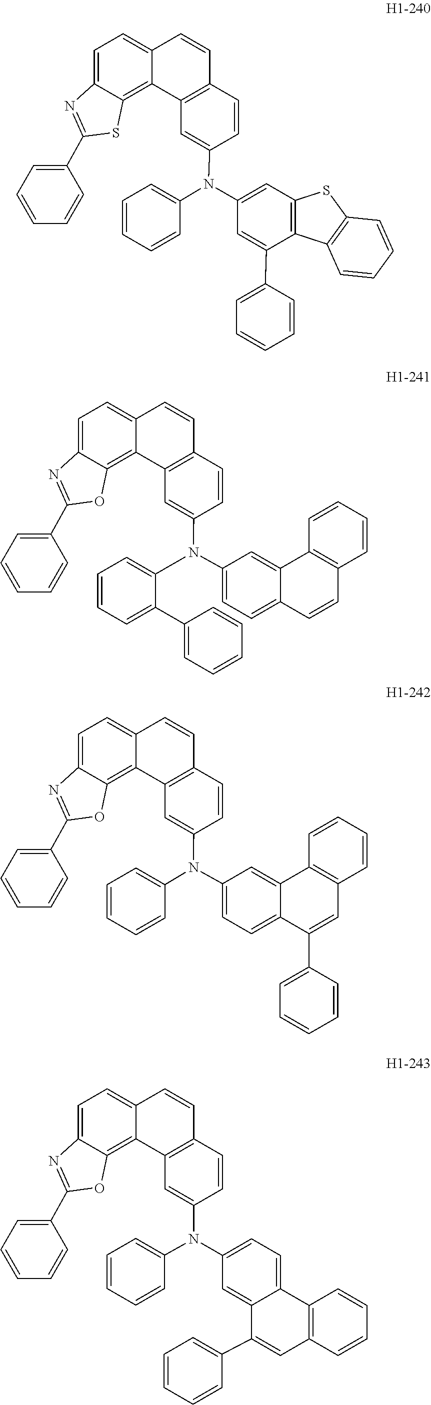 [SMILES: c1ccc(-c2nc3ccc4ccc5ccc(N(c6ccccc6)c6cc(-c7ccccc7)c7c(c6)sc6ccccc67)cc5c4c3s2)cc1 ; c1ccc(-c2nc3ccc4ccc5ccc(N(c6ccc7ccc8ccccc8c7c6)c6ccccc6-c6ccccc6)cc5c4c3o2)cc1 ; c1ccc(-c2nc3ccc4ccc5ccc(N(c6ccccc6)c6ccc7c(-c8ccccc8)cc8ccccc8c7c6)cc5c4c3o2)cc1 ; c1ccc(-c2nc3ccc4ccc5ccc(N(c6ccccc6)c6ccc7c(c6)c(-c6ccccc6)cc6ccccc67)cc5c4c3o2)cc1]

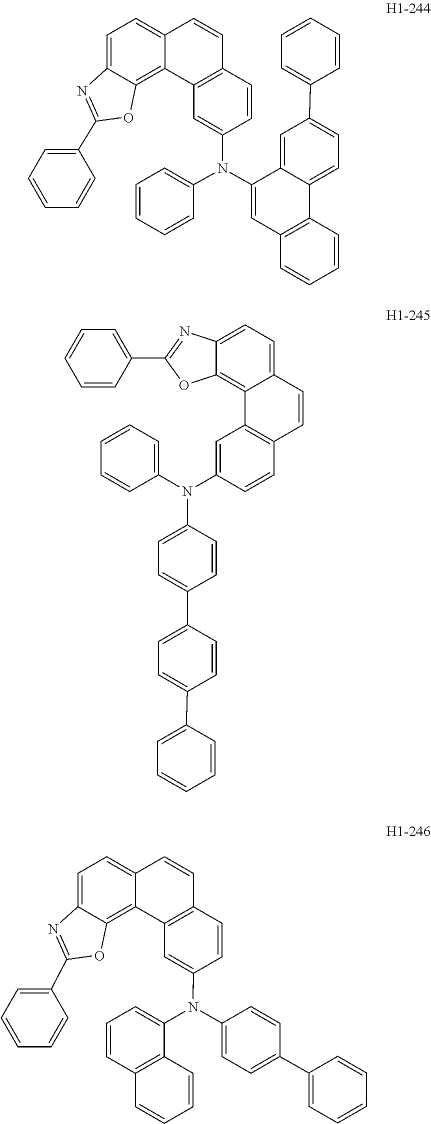 [SMILES: c1ccc(-c2ccc3c(c2)c(N(c2ccccc2)c2ccc4ccc5ccc6nc(-c7ccccc7)oc6c5c4c2)cc2ccccc23)cc1 ; c1ccc(-c2ccc(-c3ccc(N(c4ccccc4)c4ccc5ccc6ccc7nc(-c8ccccc8)oc7c6c5c4)cc3)cc2)cc1 ; c1ccc(-c2ccc(N(c3ccc4ccc5ccc6nc(-c7ccccc7)oc6c5c4c3)c3cccc4ccccc34)cc2)cc1]

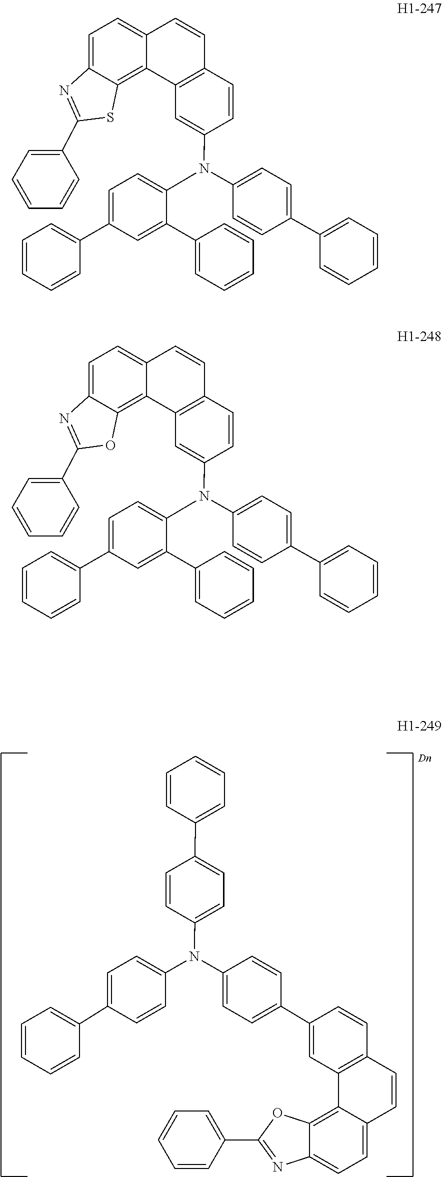

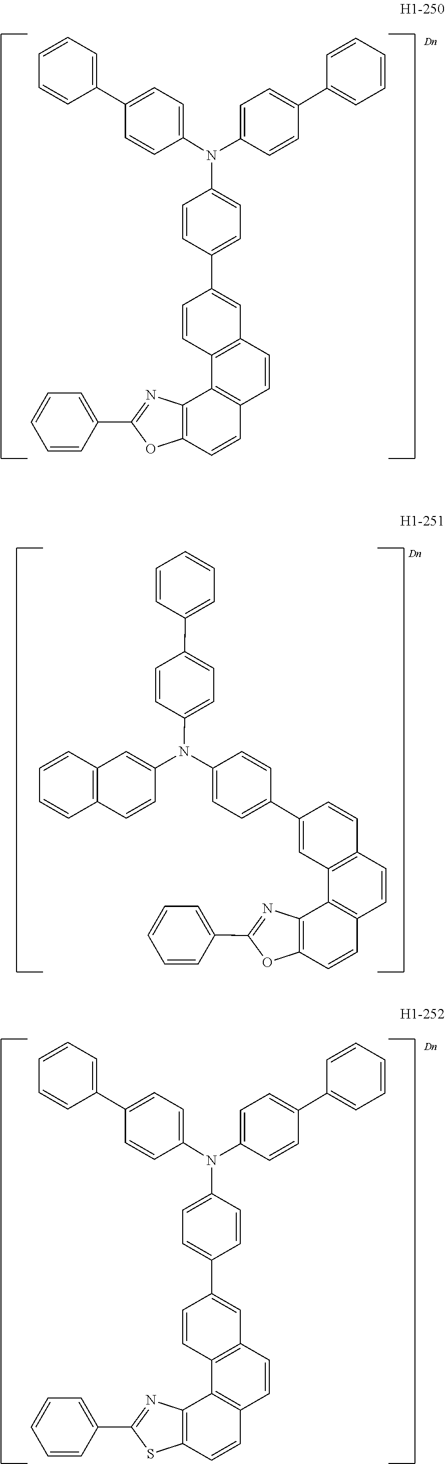

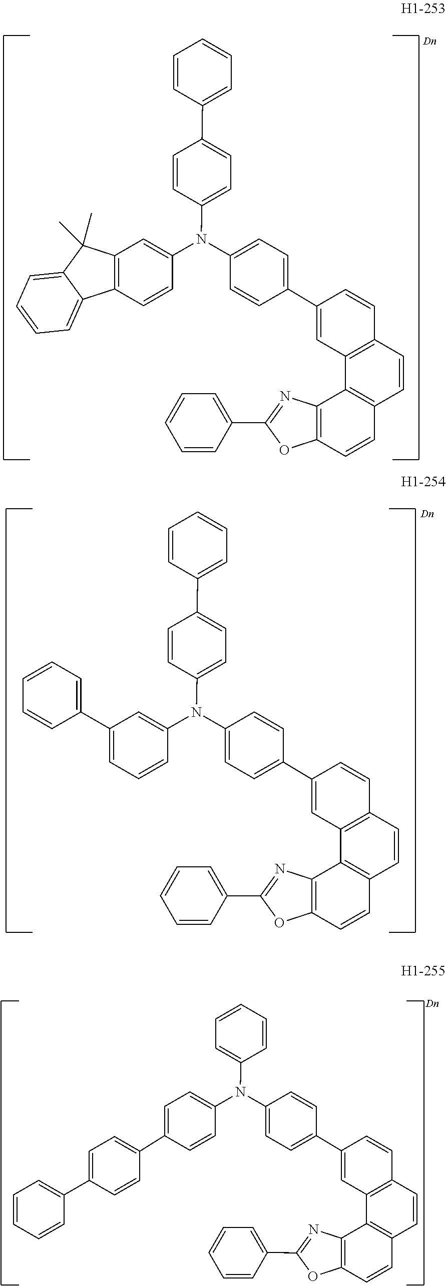

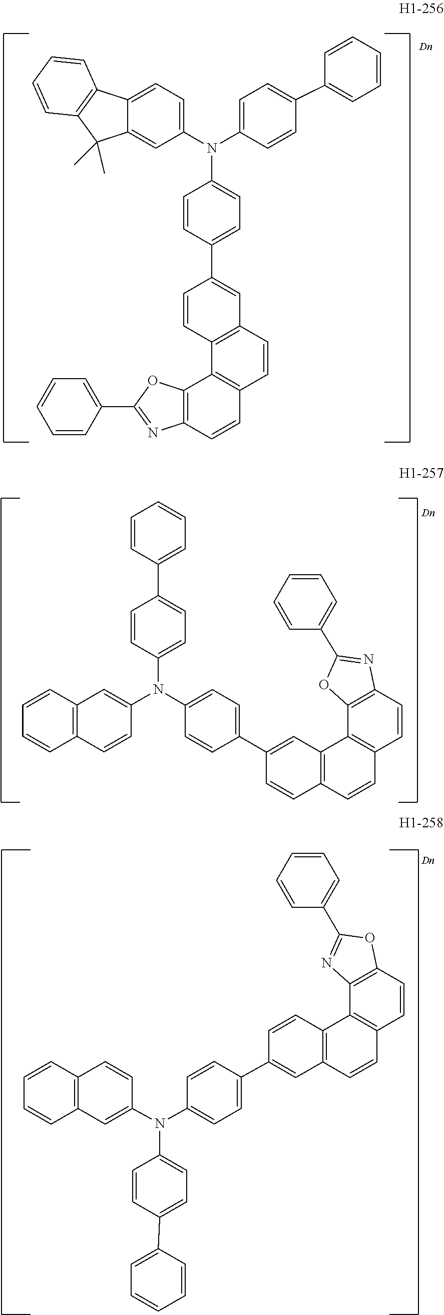

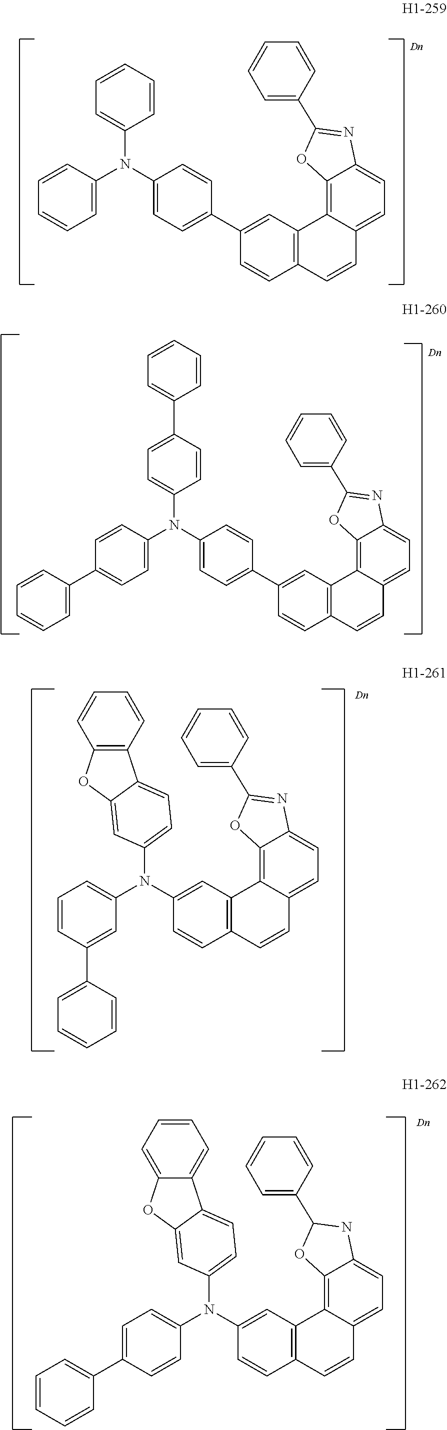

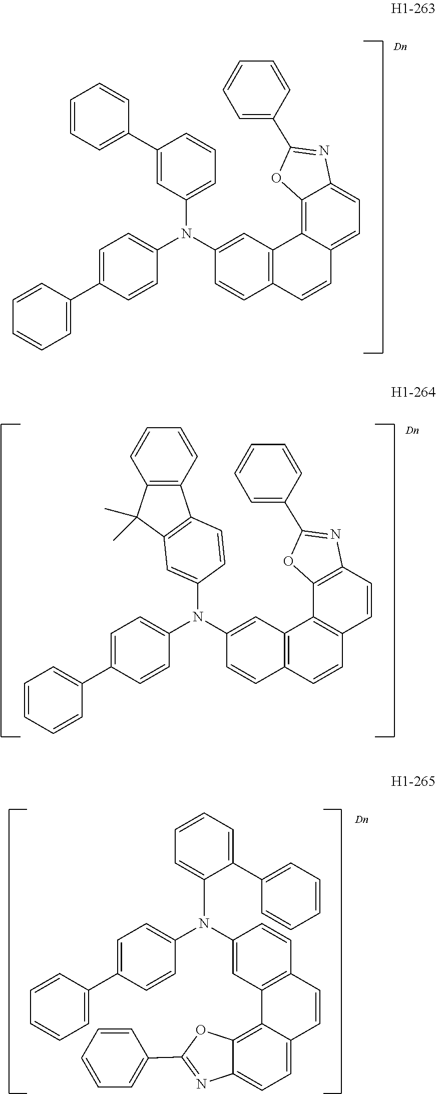

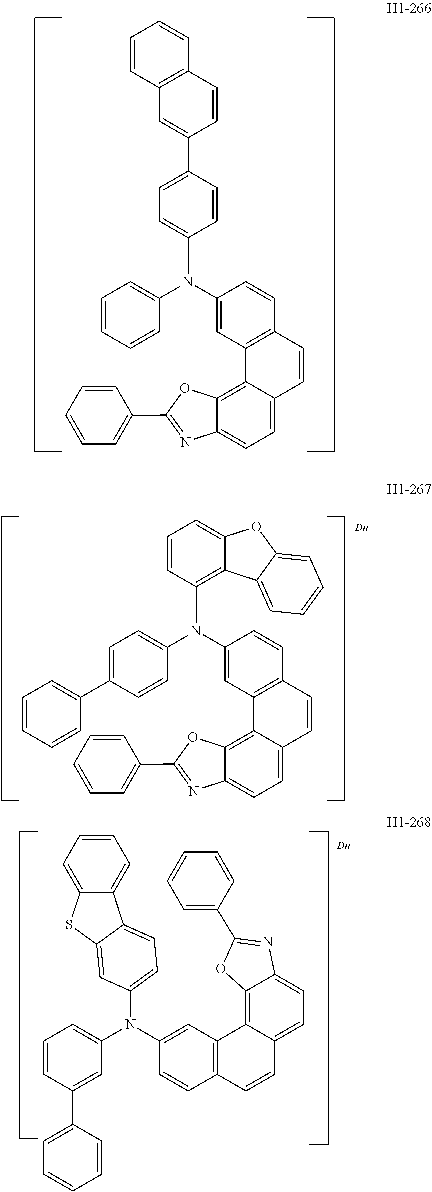

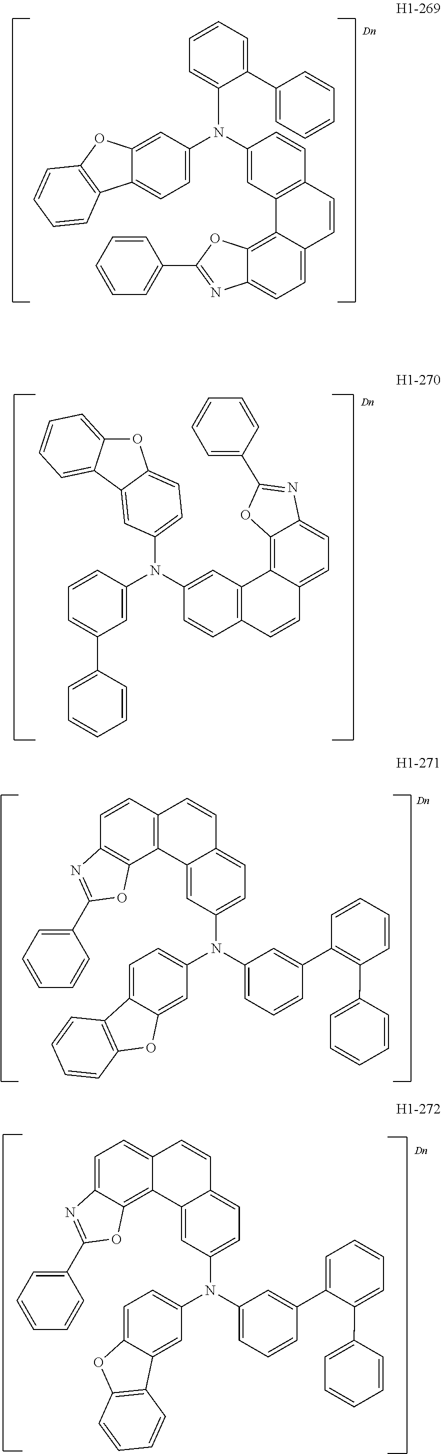

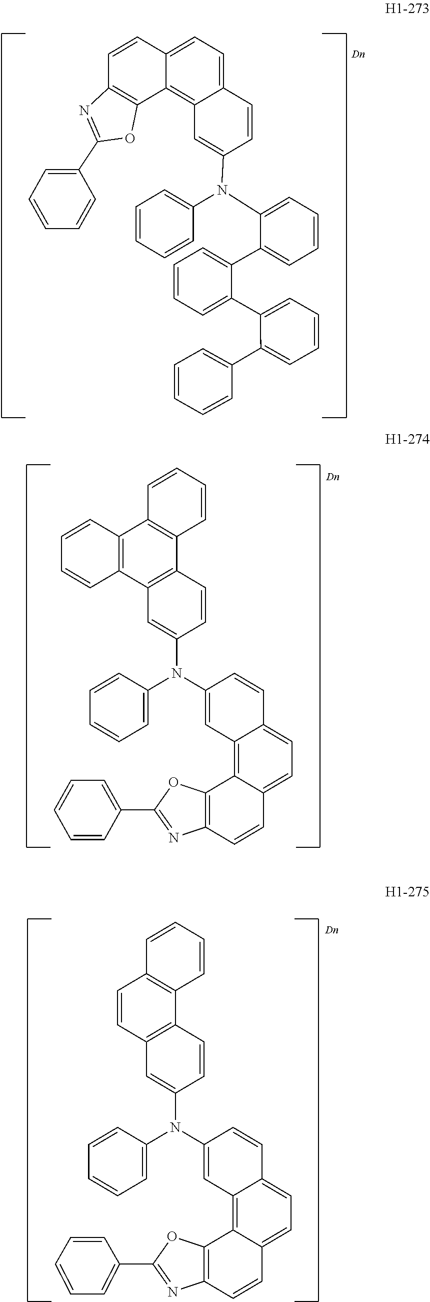

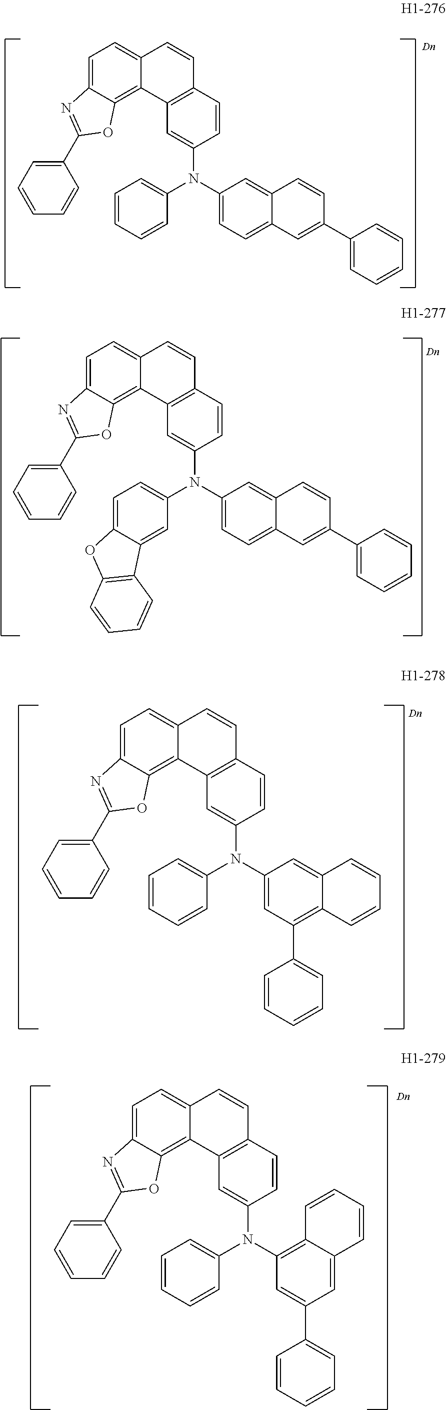

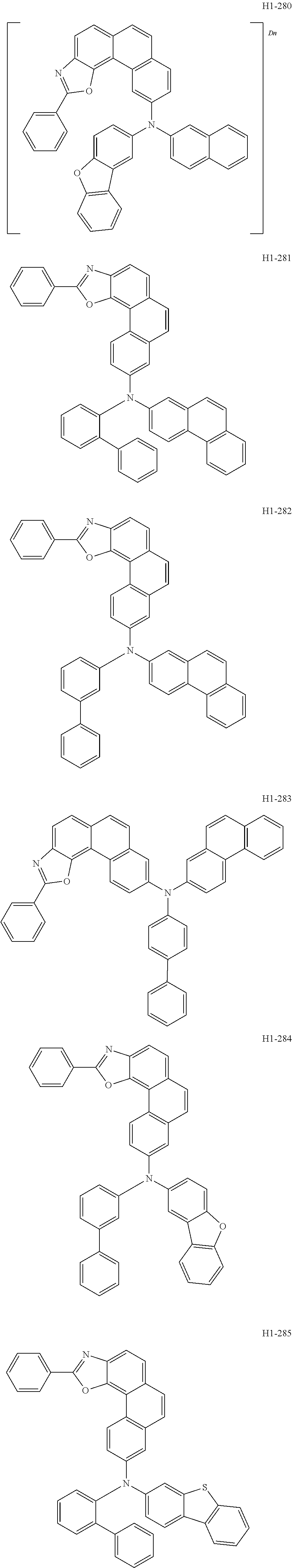

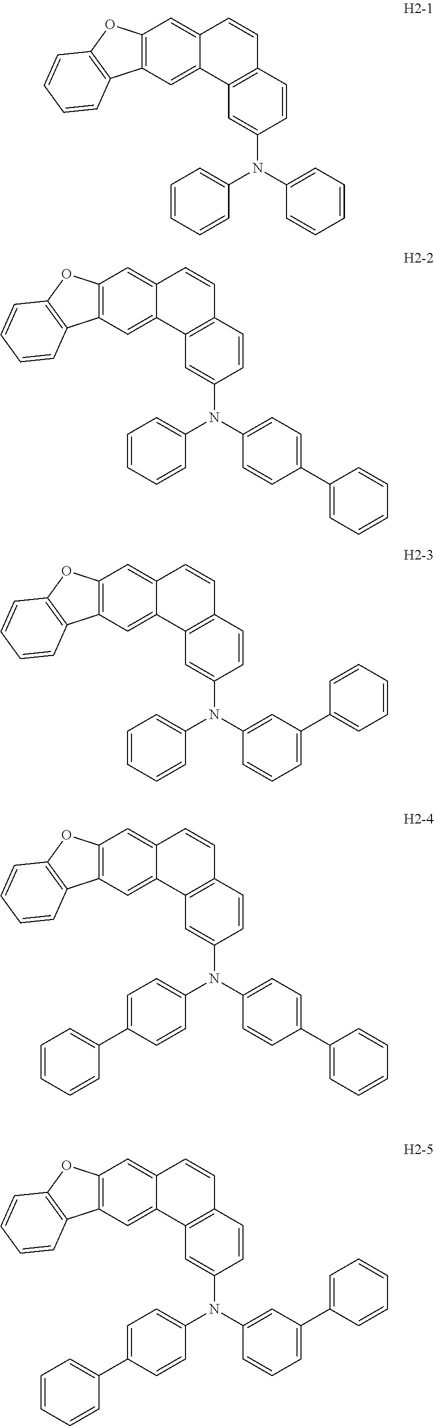

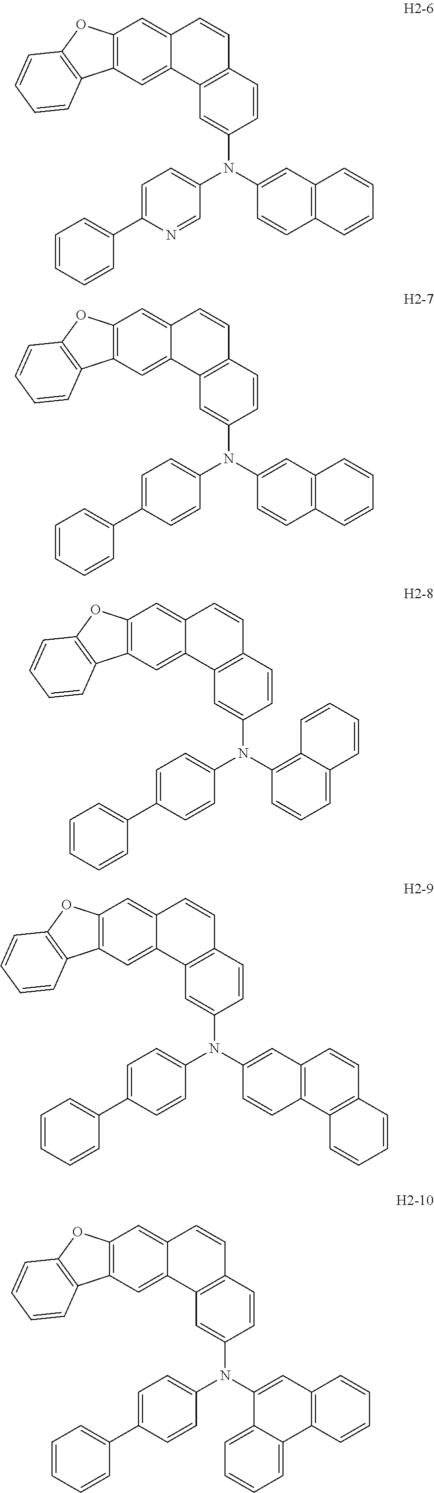

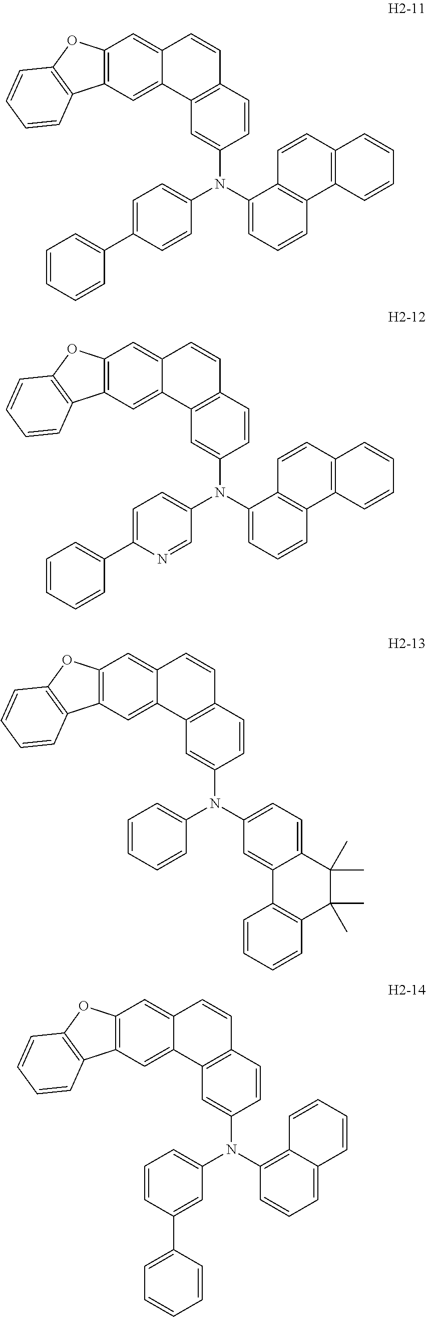 [SMILES: c1ccc(-c2ccc(N(c3ccc4ccc5cc6oc7ccccc7c6cc5c4c3)c3cccc4c3ccc3ccccc34)cc2)cc1 ; c1ccc(-c2ccc(N(c3ccc4ccc5cc6oc7ccccc7c6cc5c4c3)c3cccc4c3ccc3ccccc34)cn2)cc1 ; CC1(C)c2ccccc2-c2cc(N(c3ccccc3)c3ccc4ccc5cc6oc7ccccc7c6cc5c4c3)ccc2C1(C)C ; c1ccc(-c2cccc(N(c3ccc4ccc5cc6oc7ccccc7c6cc5c4c3)c3cccc4ccccc34)c2)cc1]

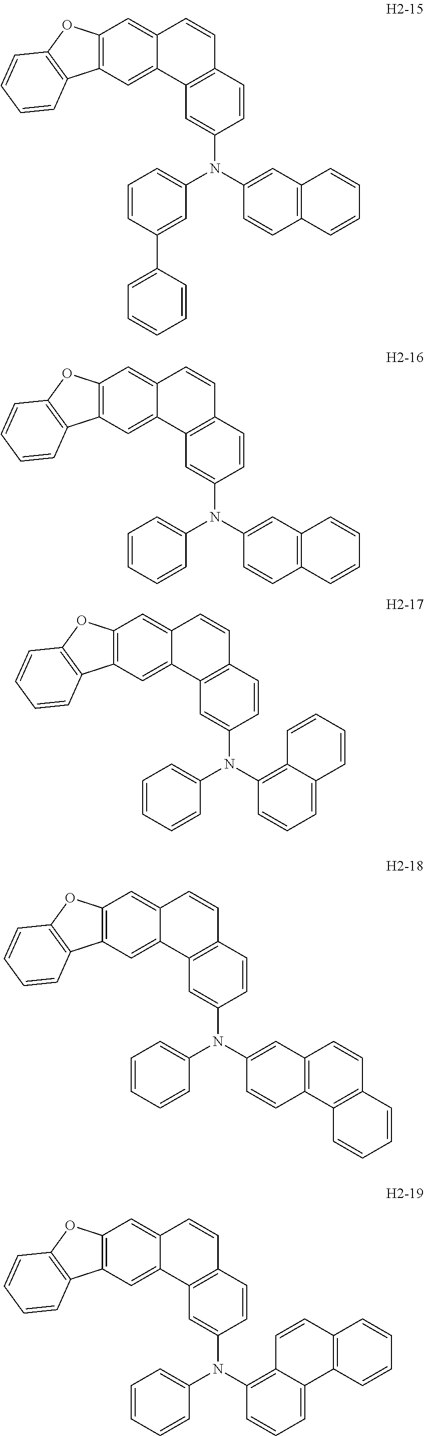

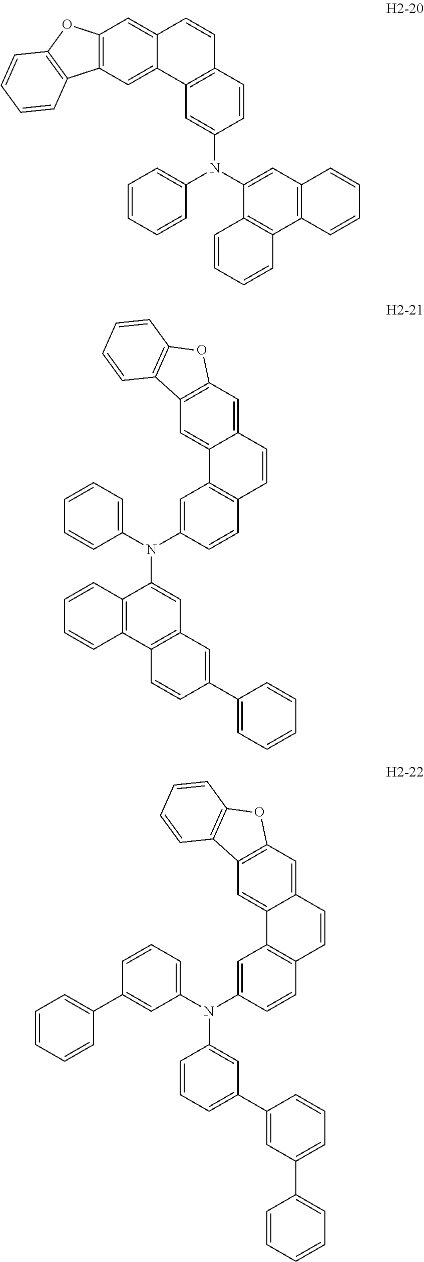 [SMILES: c1ccc(N(c2ccc3ccc4cc5oc6ccccc6c5cc4c3c2)c2cc3ccccc3c3ccccc23)cc1 ; c1ccc(-c2ccc3c(c2)cc(N(c2ccccc2)c2ccc4ccc5cc6oc7ccccc7c6cc5c4c2)c2ccccc23)cc1 ; c1ccc(-c2cccc(-c3cccc(N(c4cccc(-c5ccccc5)c4)c4ccc5ccc6cc7oc8ccccc8c7cc6c5c4)c3)c2)cc1]

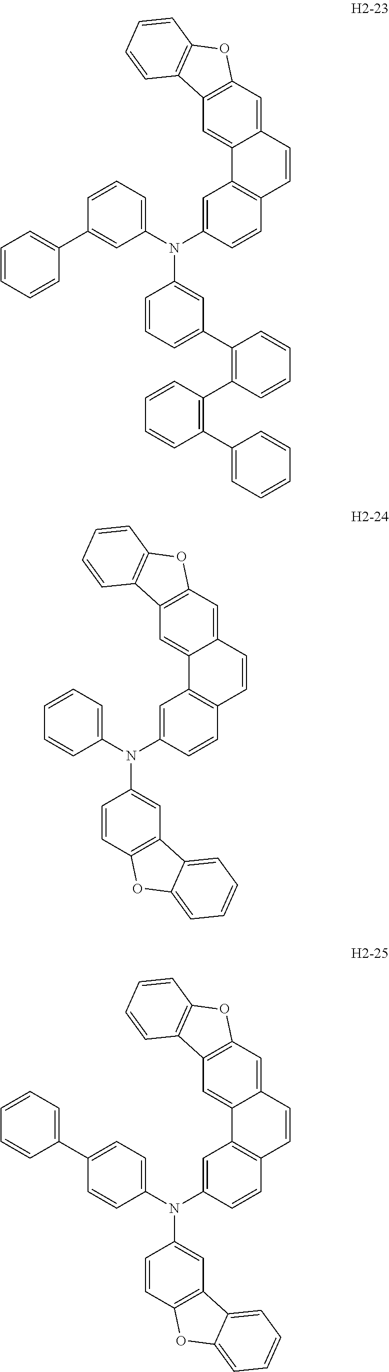 [SMILES: c1ccc(-c2cccc(N(c3cccc(-c4ccccc4-c4ccccc4-c4ccccc4)c3)c3ccc4ccc5cc6oc7ccccc7c6cc5c4c3)c2)cc1 ; c1ccc(N(c2ccc3ccc4cc5oc6ccccc6c5cc4c3c2)c2ccc3oc4ccccc4c3c2)cc1 ; c1ccc(-c2ccc(N(c3ccc4ccc5cc6oc7ccccc7c6cc5c4c3)c3ccc4oc5ccccc5c4c3)cc2)cc1]

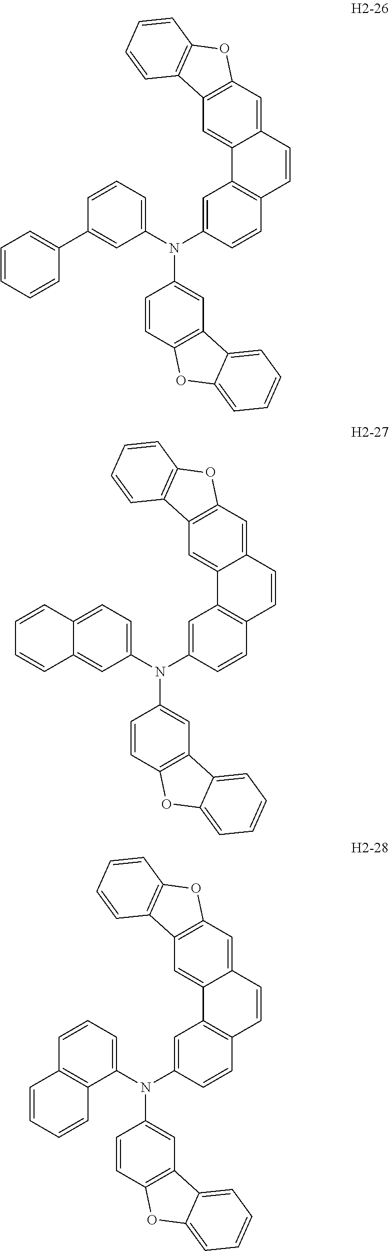 [SMILES: c1ccc(-c2cccc(N(c3ccc4ccc5cc6oc7ccccc7c6cc5c4c3)c3ccc4oc5ccccc5c4c3)c2)cc1 ; c1ccc2cc(N(c3ccc4ccc5cc6oc7ccccc7c6cc5c4c3)c3ccc4oc5ccccc5c4c3)ccc2c1 ; c1ccc2c(N(c3ccc4ccc5cc6oc7ccccc7c6cc5c4c3)c3ccc4oc5ccccc5c4c3)cccc2c1]

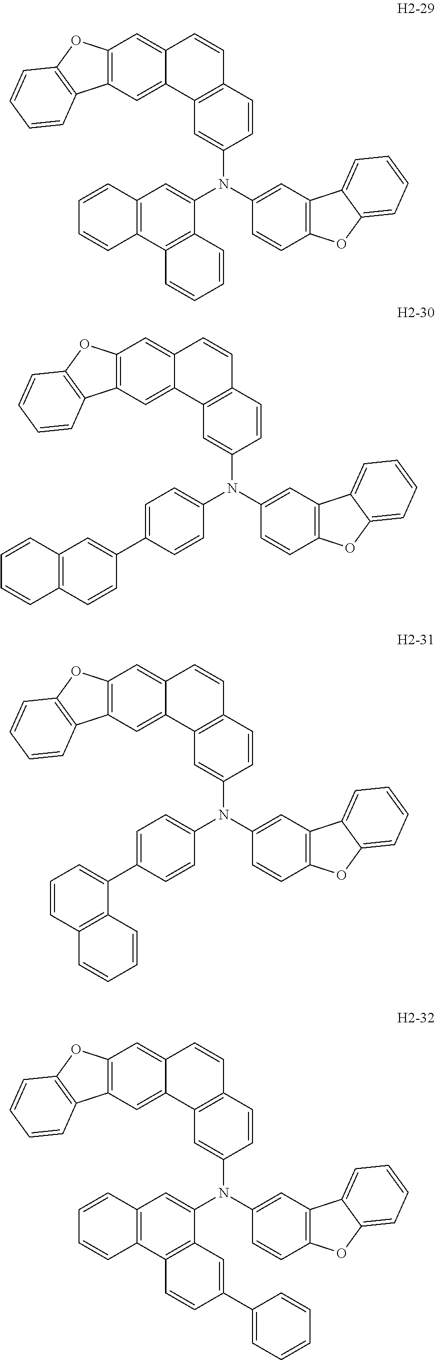

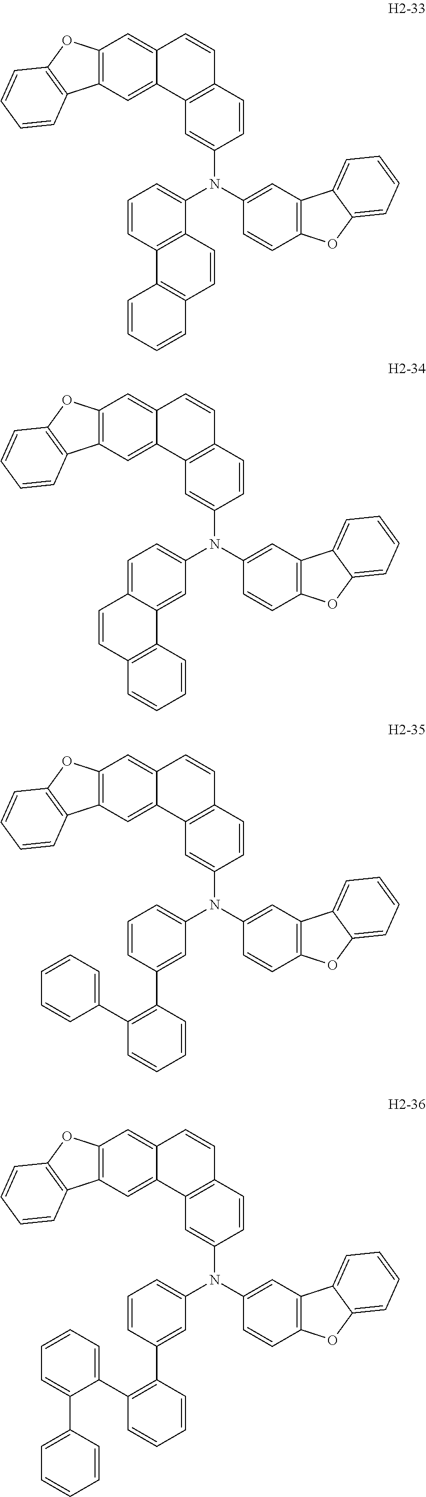 [SMILES: c1ccc2c(c1)ccc1c(N(c3ccc4ccc5cc6oc7ccccc7c6cc5c4c3)c3ccc4oc5ccccc5c4c3)cccc12 ; c1ccc2c(c1)ccc1ccc(N(c3ccc4ccc5cc6oc7ccccc7c6cc5c4c3)c3ccc4oc5ccccc5c4c3)cc12 ; c1ccc(-c2ccccc2-c2cccc(N(c3ccc4ccc5cc6oc7ccccc7c6cc5c4c3)c3ccc4oc5ccccc5c4c3)c2)cc1 ; c1ccc(-c2ccccc2-c2ccccc2-c2cccc(N(c3ccc4ccc5cc6oc7ccccc7c6cc5c4c3)c3ccc4oc5ccccc5c4c3)c2)cc1]

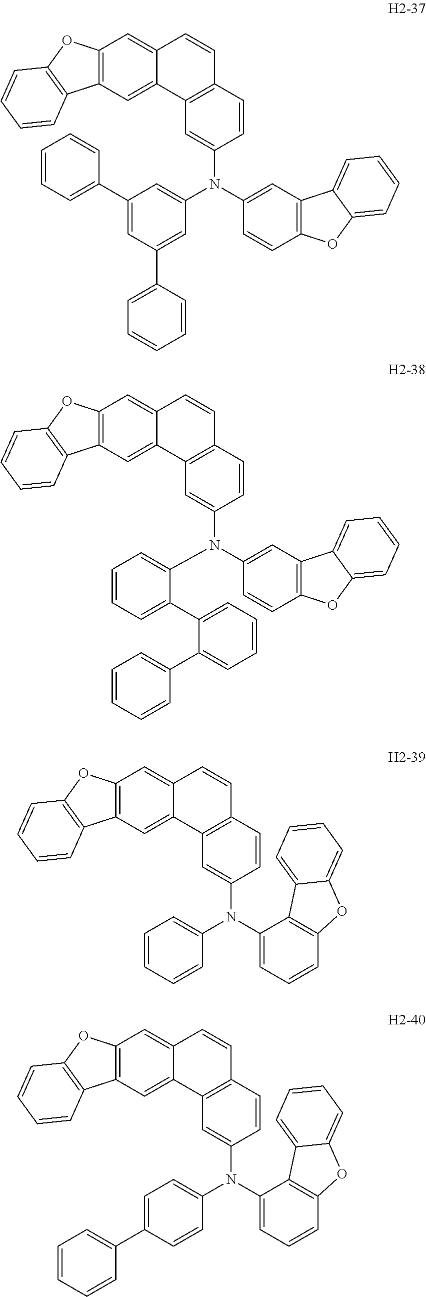 [SMILES: c1ccc(-c2cc(-c3ccccc3)cc(N(c3ccc4ccc5cc6oc7ccccc7c6cc5c4c3)c3ccc4oc5ccccc5c4c3)c2)cc1 ; c1ccc(-c2ccccc2-c2ccccc2N(c2ccc3ccc4cc5oc6ccccc6c5cc4c3c2)c2ccc3oc4ccccc4c3c2)cc1 ; c1ccc(N(c2ccc3ccc4cc5oc6ccccc6c5cc4c3c2)c2cccc3oc4ccccc4c23)cc1 ; c1ccc(-c2ccc(N(c3ccc4ccc5cc6oc7ccccc7c6cc5c4c3)c3cccc4oc5ccccc5c34)cc2)cc1]

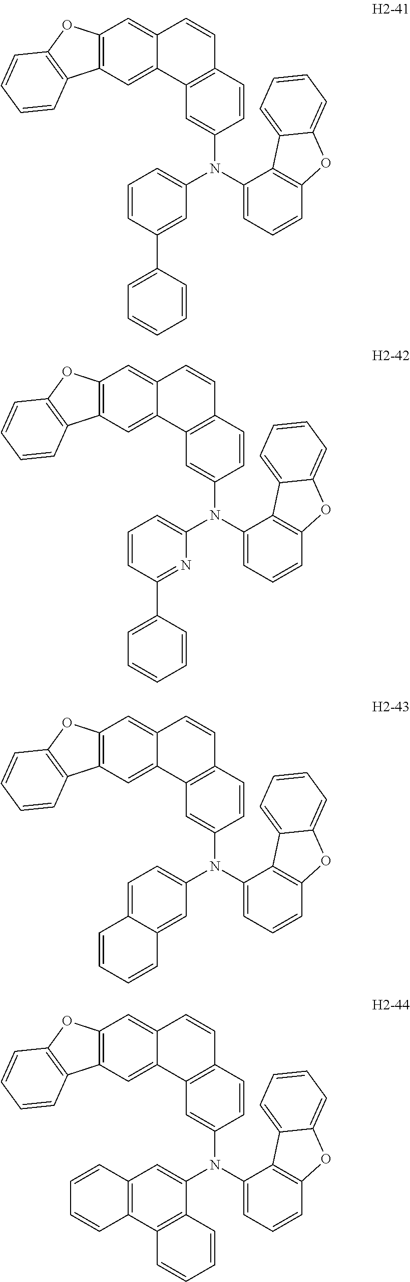

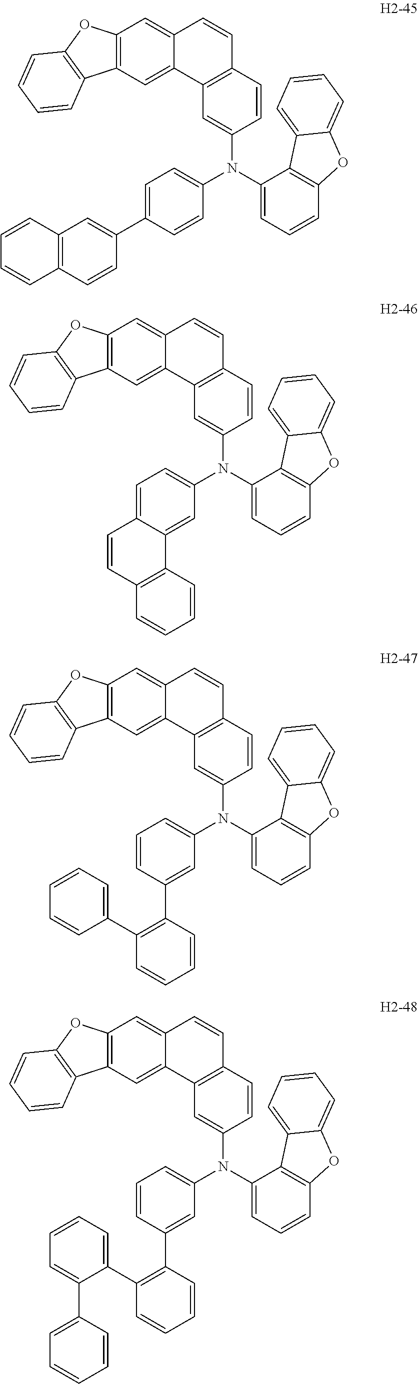

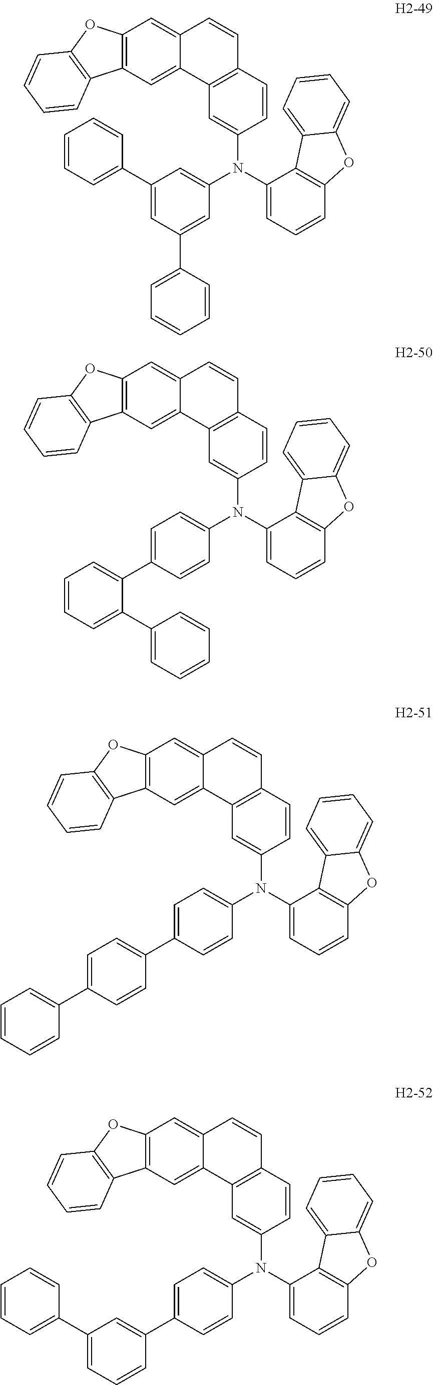

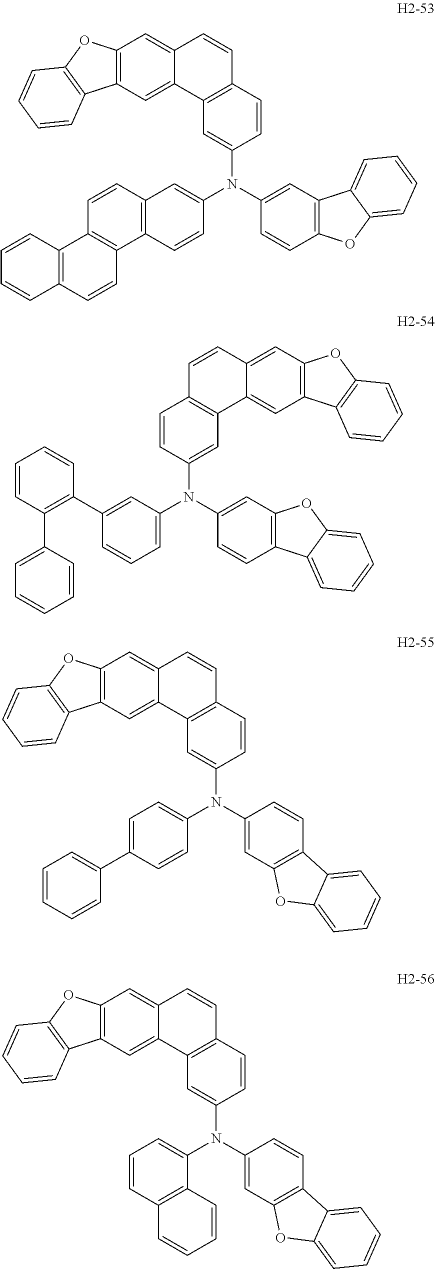 [SMILES: c1ccc2c(c1)ccc1c3ccc(N(c4ccc5ccc6cc7oc8ccccc8c7cc6c5c4)c4ccc5oc6ccccc6c5c4)cc3ccc21 ; c1ccc(-c2ccccc2-c2cccc(N(c3ccc4c(c3)oc3ccccc34)c3ccc4ccc5cc6oc7ccccc7c6cc5c4c3)c2)cc1 ; c1ccc(-c2ccc(N(c3ccc4c(c3)oc3ccccc34)c3ccc4ccc5cc6oc7ccccc7c6cc5c4c3)cc2)cc1 ; c1ccc2c(N(c3ccc4c(c3)oc3ccccc34)c3ccc4ccc5cc6oc7ccccc7c6cc5c4c3)cccc2c1]

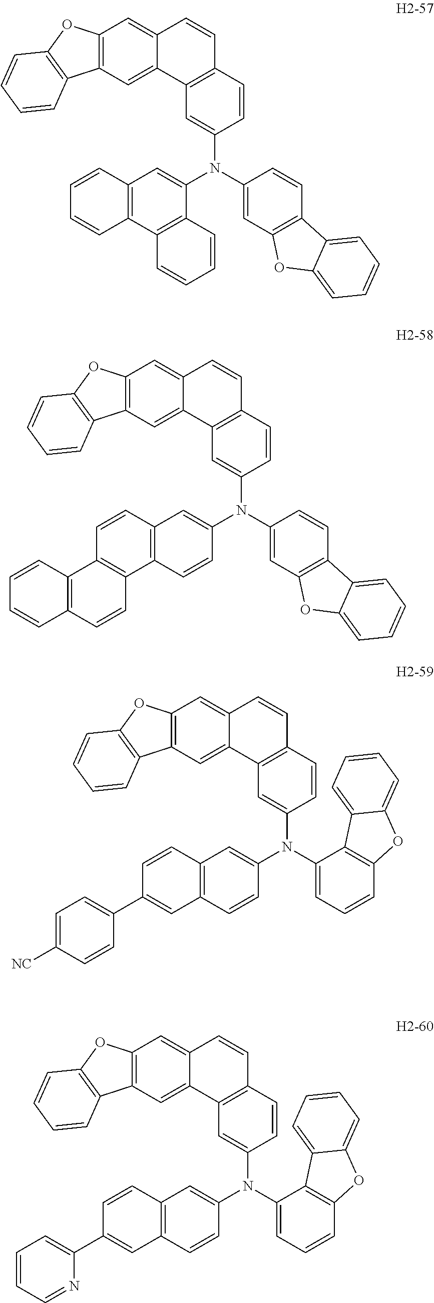 [SMILES: c1ccc2c(c1)cc(N(c1ccc3c(c1)oc1ccccc13)c1ccc3ccc4cc5oc6ccccc6c5cc4c3c1)c1ccccc12 ; c1ccc2c(c1)ccc1c3ccc(N(c4ccc5c(c4)oc4ccccc45)c4ccc5ccc6cc7oc8ccccc8c7cc6c5c4)cc3ccc21 ; N#Cc1ccc(-c2ccc3cc(N(c4ccc5ccc6cc7oc8ccccc8c7cc6c5c4)c4cccc5oc6ccccc6c45)ccc3c2)cc1 ; c1ccc(-c2ccc3cc(N(c4ccc5ccc6cc7oc8ccccc8c7cc6c5c4)c4cccc5oc6ccccc6c45)ccc3c2)nc1]

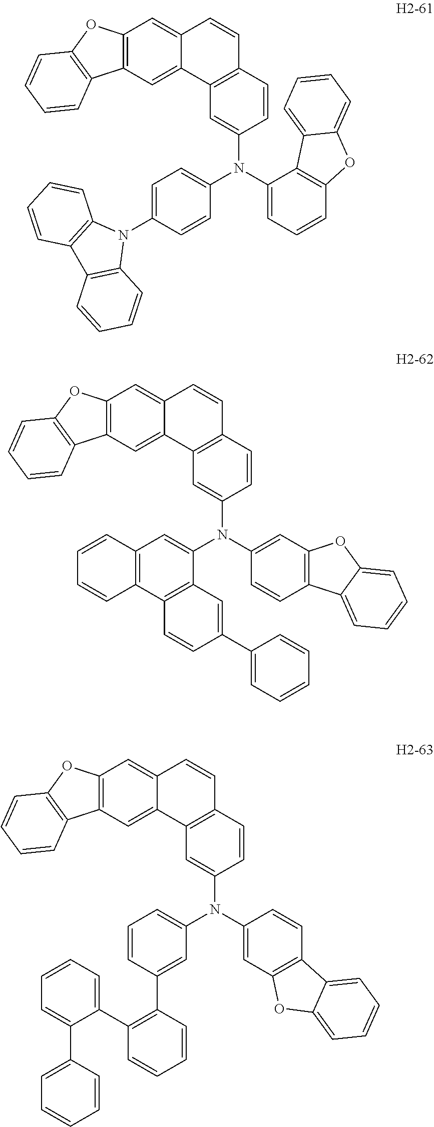 [SMILES: c1ccc2c(c1)oc1cc3ccc4ccc(N(c5ccc(-n6c7ccccc7c7ccccc76)cc5)c5cccc6oc7ccccc7c56)cc4c3cc12 ; c1ccc(-c2ccc3c(c2)c(N(c2ccc4c(c2)oc2ccccc24)c2ccc4ccc5cc6oc7ccccc7c6cc5c4c2)cc2ccccc23)cc1 ; c1ccc(-c2ccccc2-c2ccccc2-c2cccc(N(c3ccc4c(c3)oc3ccccc34)c3ccc4ccc5cc6oc7ccccc7c6cc5c4c3)c2)cc1]

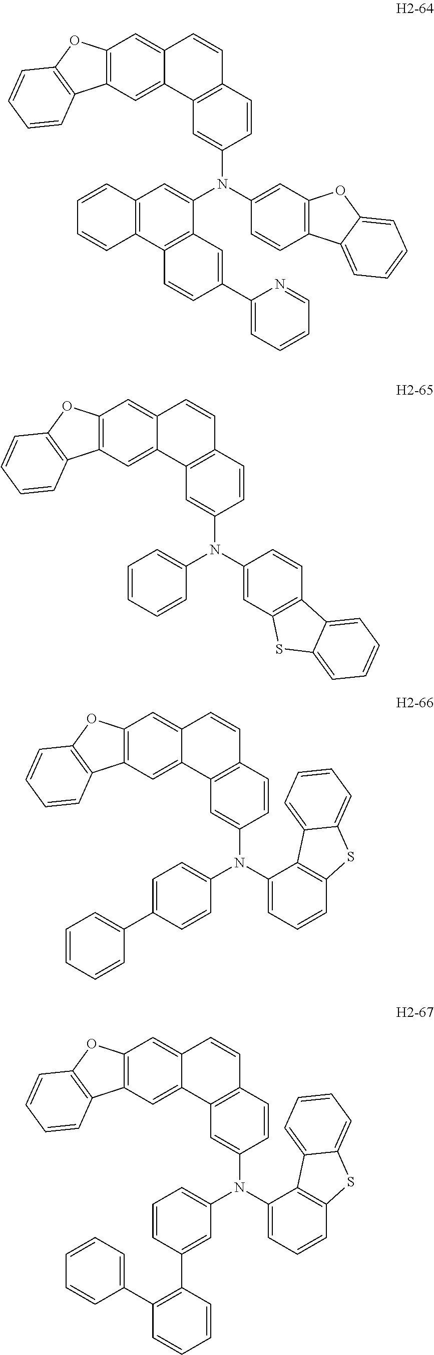 [SMILES: c1ccc(-c2ccc3c(c2)c(N(c2ccc4c(c2)oc2ccccc24)c2ccc4ccc5cc6oc7ccccc7c6cc5c4c2)cc2ccccc23)nc1 ; c1ccc(N(c2ccc3c(c2)sc2ccccc23)c2ccc3ccc4cc5oc6ccccc6c5cc4c3c2)cc1 ; c1ccc(-c2ccc(N(c3ccc4ccc5cc6oc7ccccc7c6cc5c4c3)c3cccc4sc5ccccc5c34)cc2)cc1 ; c1ccc(-c2ccccc2-c2cccc(N(c3ccc4ccc5cc6oc7ccccc7c6cc5c4c3)c3cccc4sc5ccccc5c34)c2)cc1]

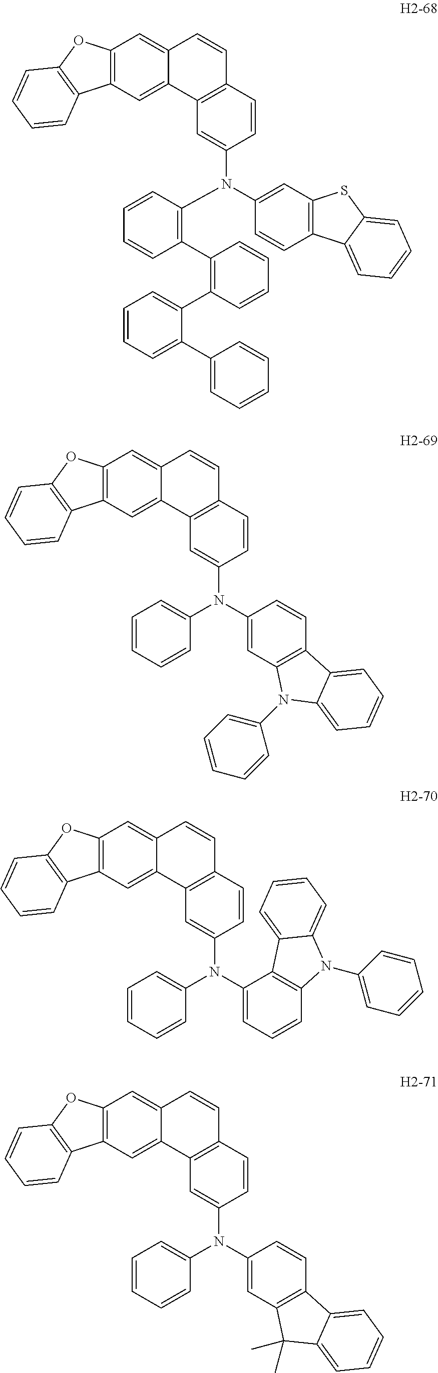 [SMILES: c1ccc(-c2ccccc2-c2ccccc2-c2ccccc2N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2ccc3c4ccccc4n(-c4ccccc4)c3c2)cc1 ; c1ccc(N(c2ccc3ccc4cc5oc6ccccc6c5cc4c3c2)c2cccc3c2c2ccccc2n3-c2ccccc2)cc1 ; CC1(C)c2ccccc2-c2ccc(N(c3ccccc3)c3ccc4ccc5cc6oc7ccccc7c6cc5c4c3)cc21]

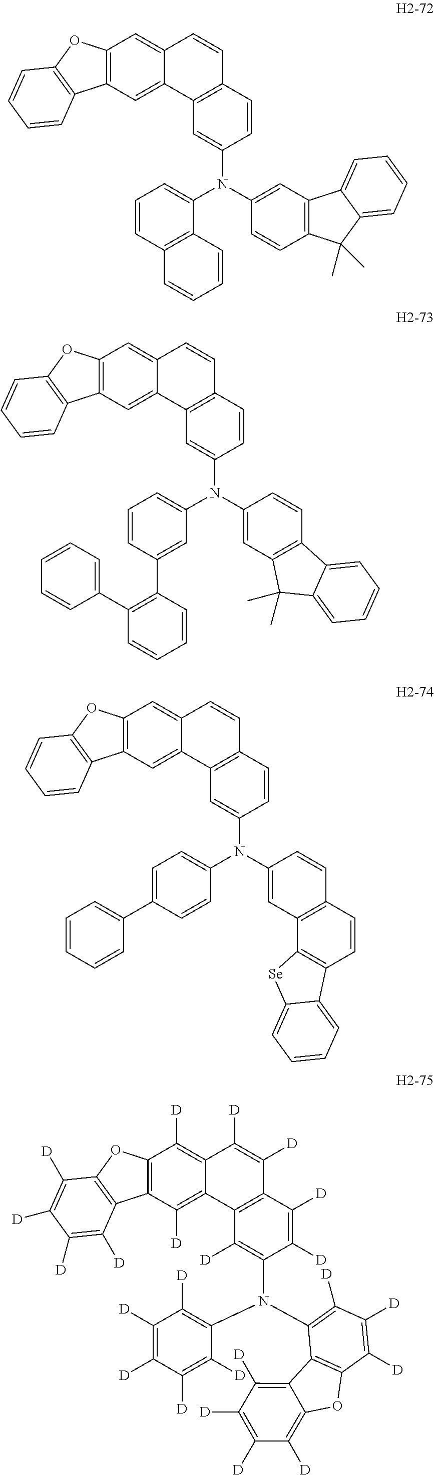

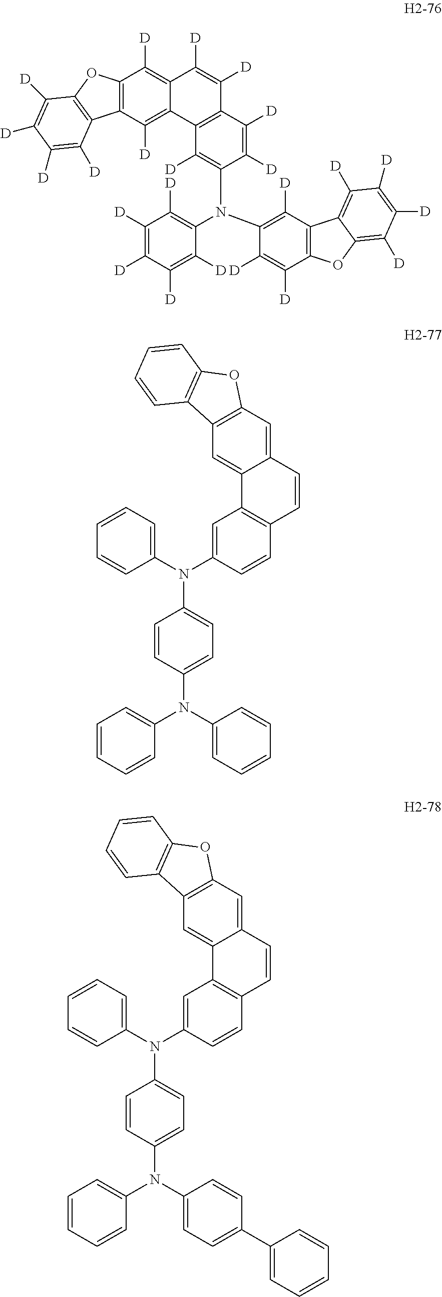

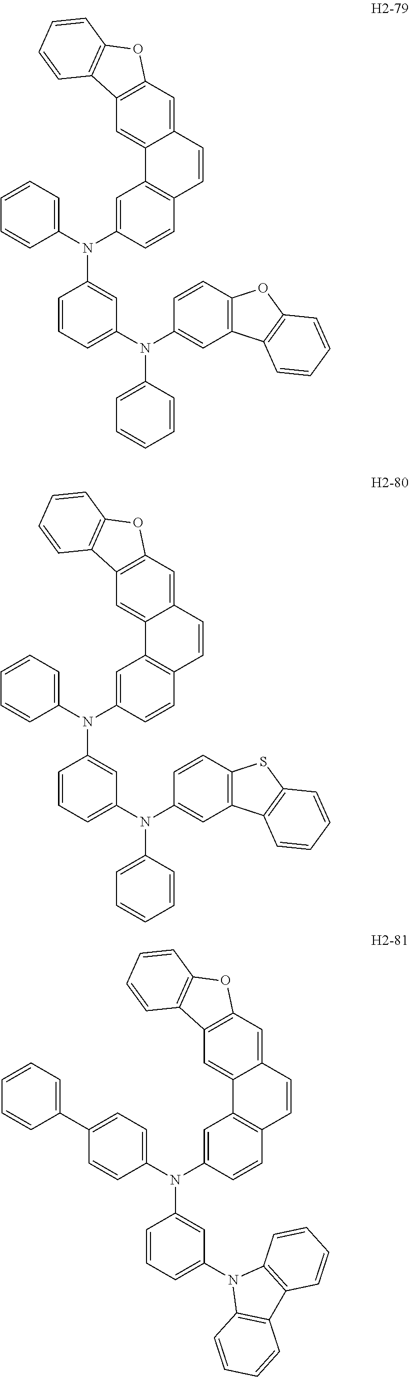

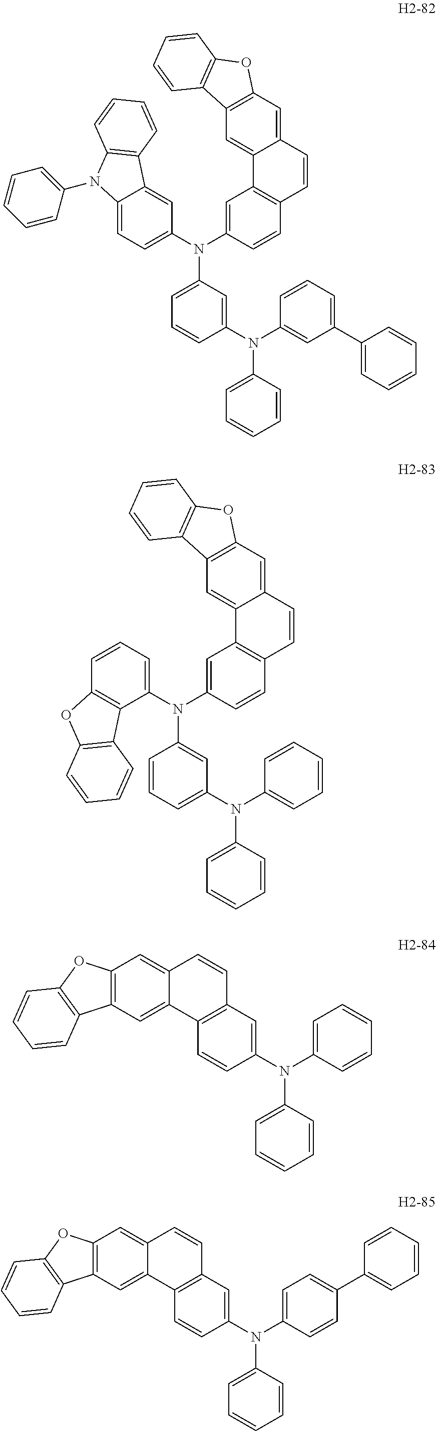 [SMILES: c1ccc(-c2cccc(N(c3ccccc3)c3cccc(N(c4ccc5ccc6cc7oc8ccccc8c7cc6c5c4)c4ccc5c(c4)c4ccccc4n5-c4ccccc4)c3)c2)cc1 ; c1ccc(N(c2ccccc2)c2cccc(N(c3ccc4ccc5cc6oc7ccccc7c6cc5c4c3)c3cccc4oc5ccccc5c34)c2)cc1 ; c1ccc(N(c2ccccc2)c2ccc3c(ccc4cc5oc6ccccc6c5cc43)c2)cc1 ; c1ccc(-c2ccc(N(c3ccccc3)c3ccc4c(ccc5cc6oc7ccccc7c6cc54)c3)cc2)cc1]

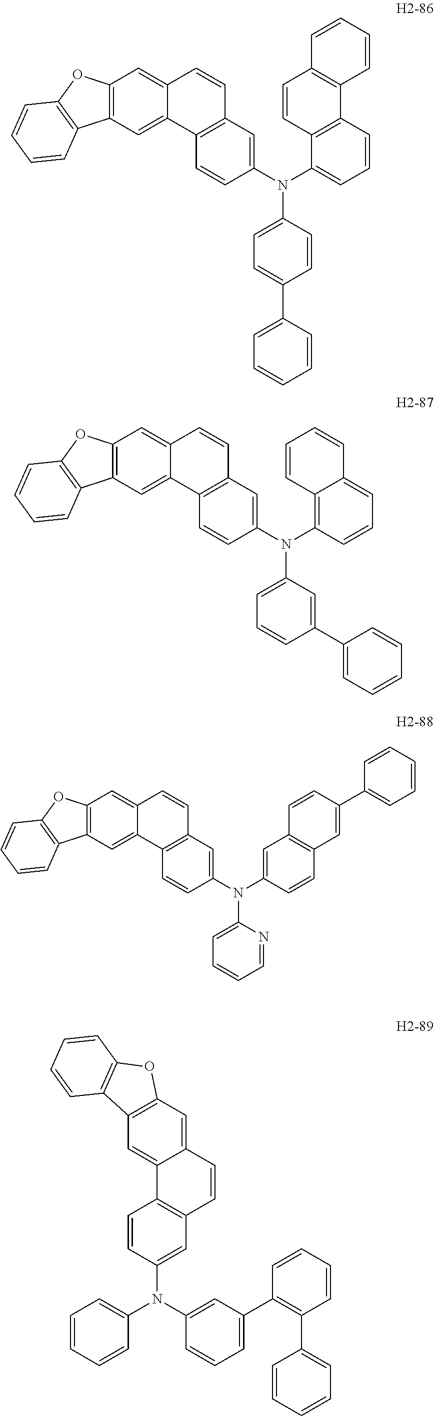 [SMILES: c1ccc(-c2ccc(N(c3ccc4c(ccc5cc6oc7ccccc7c6cc54)c3)c3cccc4c3ccc3ccccc34)cc2)cc1 ; c1ccc(-c2cccc(N(c3ccc4c(ccc5cc6oc7ccccc7c6cc54)c3)c3cccc4ccccc34)c2)cc1 ; c1ccc(-c2ccc3cc(N(c4ccc5c(ccc6cc7oc8ccccc8c7cc65)c4)c4ccccn4)ccc3c2)cc1 ; c1ccc(-c2ccccc2-c2cccc(N(c3ccccc3)c3ccc4c(ccc5cc6oc7ccccc7c6cc54)c3)c2)cc1]

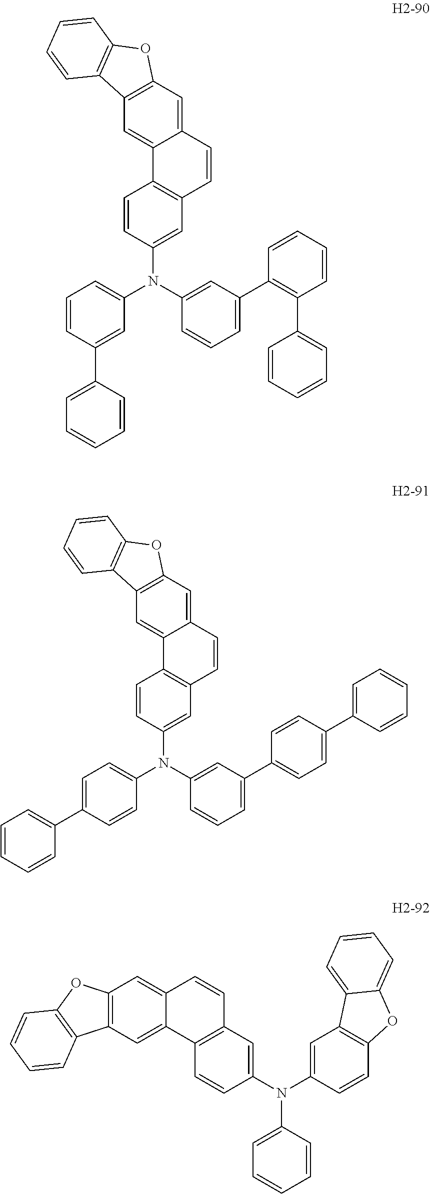 [SMILES: c1ccc(-c2cccc(N(c3cccc(-c4ccccc4-c4ccccc4)c3)c3ccc4c(ccc5cc6oc7ccccc7c6cc54)c3)c2)cc1 ; c1ccc(-c2ccc(-c3cccc(N(c4ccc(-c5ccccc5)cc4)c4ccc5c(ccc6cc7oc8ccccc8c7cc65)c4)c3)cc2)cc1 ; c1ccc(N(c2ccc3c(ccc4cc5oc6ccccc6c5cc43)c2)c2ccc3oc4ccccc4c3c2)cc1]

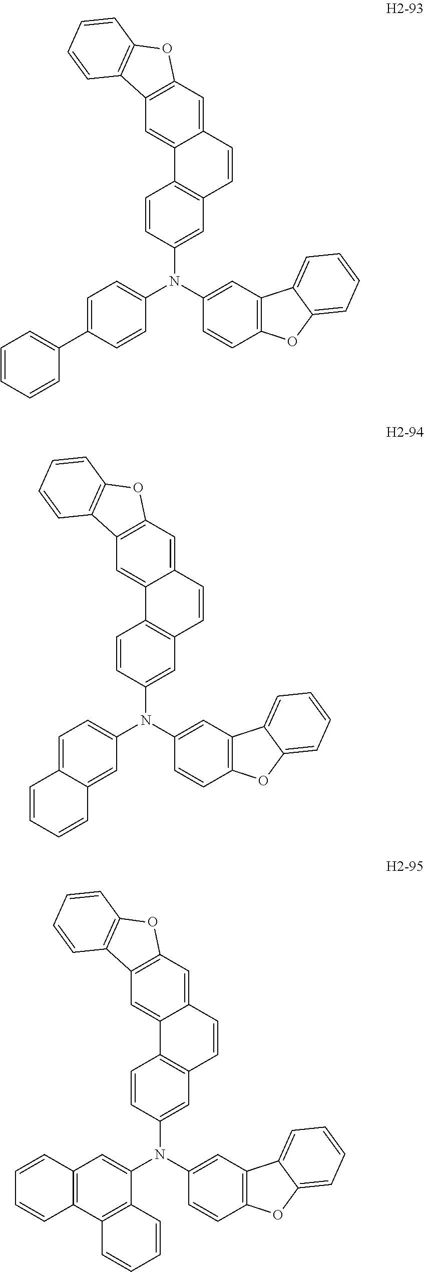 [SMILES: c1ccc(-c2ccc(N(c3ccc4c(ccc5cc6oc7ccccc7c6cc54)c3)c3ccc4oc5ccccc5c4c3)cc2)cc1 ; c1ccc2cc(N(c3ccc4c(ccc5cc6oc7ccccc7c6cc54)c3)c3ccc4oc5ccccc5c4c3)ccc2c1 ; c1ccc2c(c1)cc(N(c1ccc3c(ccc4cc5oc6ccccc6c5cc43)c1)c1ccc3oc4ccccc4c3c1)c1ccccc12]

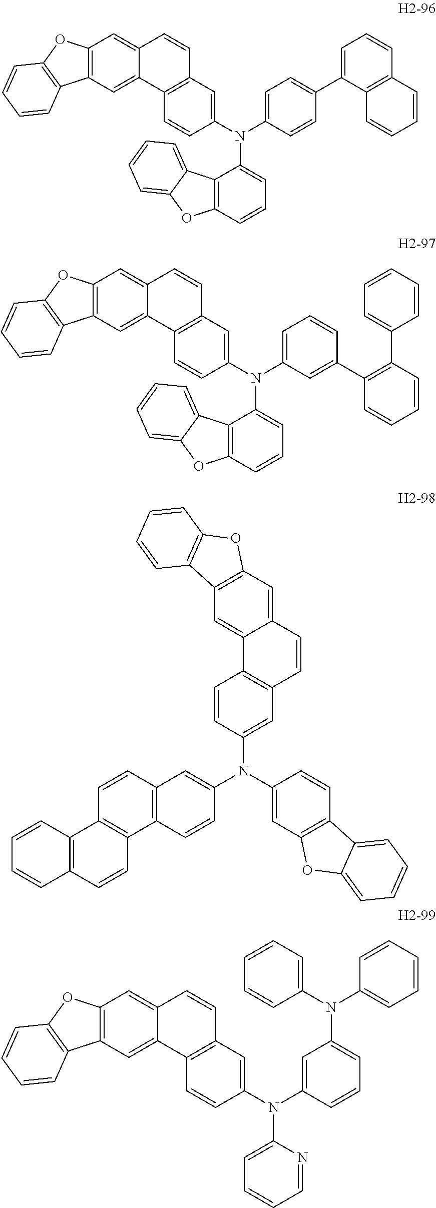 [SMILES: c1ccc2c(-c3ccc(N(c4ccc5c(ccc6cc7oc8ccccc8c7cc65)c4)c4cccc5oc6ccccc6c45)cc3)cccc2c1 ; c1ccc(-c2ccccc2-c2cccc(N(c3ccc4c(ccc5cc6oc7ccccc7c6cc54)c3)c3cccc4oc5ccccc5c34)c2)cc1 ; c1ccc2c(c1)ccc1c3ccc(N(c4ccc5c(ccc6cc7oc8ccccc8c7cc65)c4)c4ccc5c(c4)oc4ccccc45)cc3ccc21 ; c1ccc(N(c2ccccc2)c2cccc(N(c3ccc4c(ccc5cc6oc7ccccc7c6cc54)c3)c3ccccn3)c2)cc1]

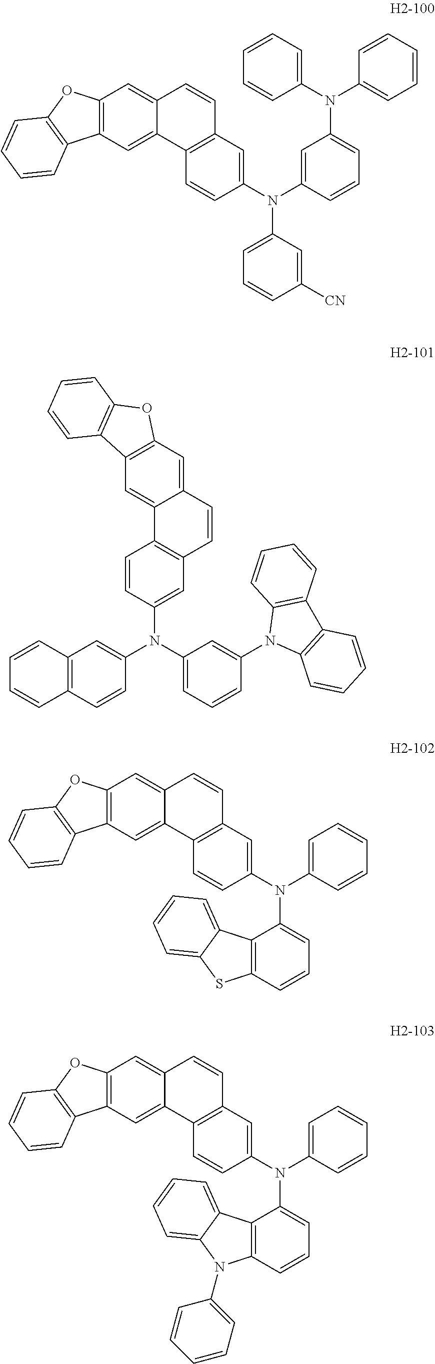 [SMILES: N#Cc1cccc(N(c2cccc(N(c3ccccc3)c3ccccc3)c2)c2ccc3c(ccc4cc5oc6ccccc6c5cc43)c2)c1 ; c1cc(N(c2ccc3ccccc3c2)c2ccc3c(ccc4cc5oc6ccccc6c5cc43)c2)cc(-n2c3ccccc3c3ccccc32)c1 ; c1ccc(N(c2ccc3c(ccc4cc5oc6ccccc6c5cc43)c2)c2cccc3sc4ccccc4c23)cc1 ; c1ccc(N(c2ccc3c(ccc4cc5oc6ccccc6c5cc43)c2)c2cccc3c2c2ccccc2n3-c2ccccc2)cc1]

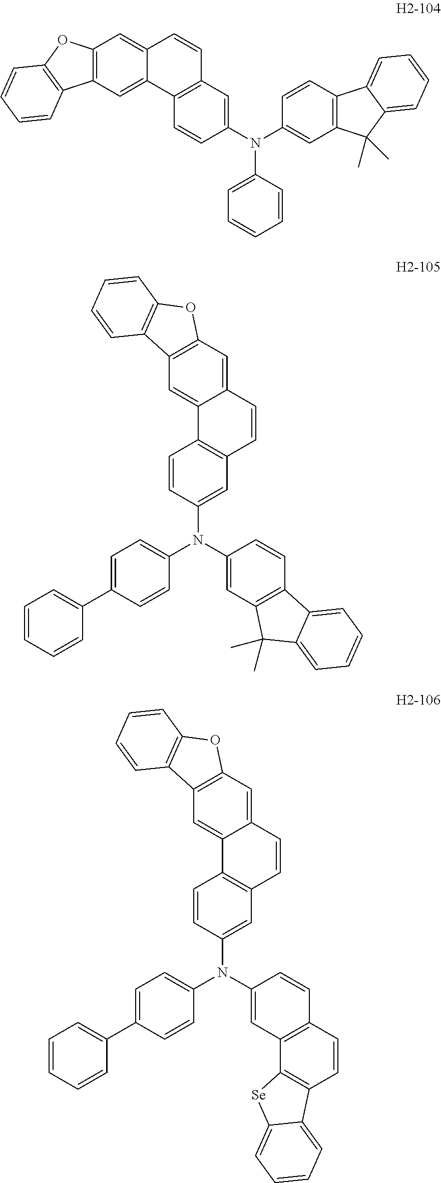 [SMILES: CC1(C)c2ccccc2-c2ccc(N(c3ccccc3)c3ccc4c(ccc5cc6oc7ccccc7c6cc54)c3)cc21 ; CC1(C)c2ccccc2-c2ccc(N(c3ccc(-c4ccccc4)cc3)c3ccc4c(ccc5cc6oc7ccccc7c6cc54)c3)cc21 ; c1ccc(-c2ccc(N(c3ccc4c(ccc5cc6oc7ccccc7c6cc54)c3)c3ccc4ccc5c6ccccc6[se]c5c4c3)cc2)cc1]

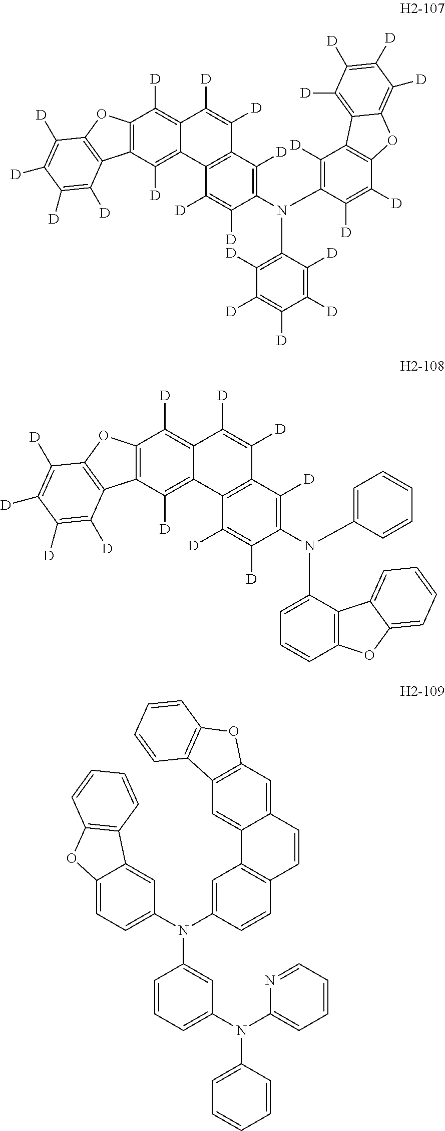 [SMILES: [2H]c1c([2H])c([2H])c(N(c2c([2H])c([2H])c3c(c2[2H])c([2H])c([2H])c2c([2H])c4oc5c([2H])c([2H])c([2H])c([2H])c5c4c([2H])c23)c2c([2H])c([2H])c3oc4c([2H])c([2H])c([2H])c([2H])c4c3c2[2H])c([2H])c1[2H] ; [2H]c1c([2H])c([2H])c2c(oc3c([2H])c4c([2H])c([2H])c5c([2H])c(N(c6ccccc6)c6cccc7oc8ccccc8c67)c([2H])c([2H])c5c4c([2H])c32)c1[2H] ; c1ccc(N(c2cccc(N(c3ccc4ccc5cc6oc7ccccc7c6cc5c4c3)c3ccc4oc5ccccc5c4c3)c2)c2ccccn2)cc1]

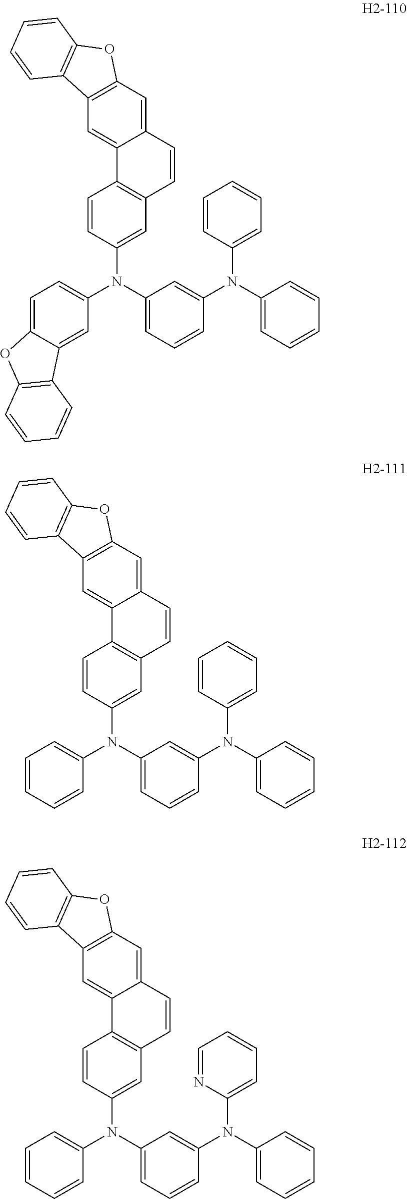

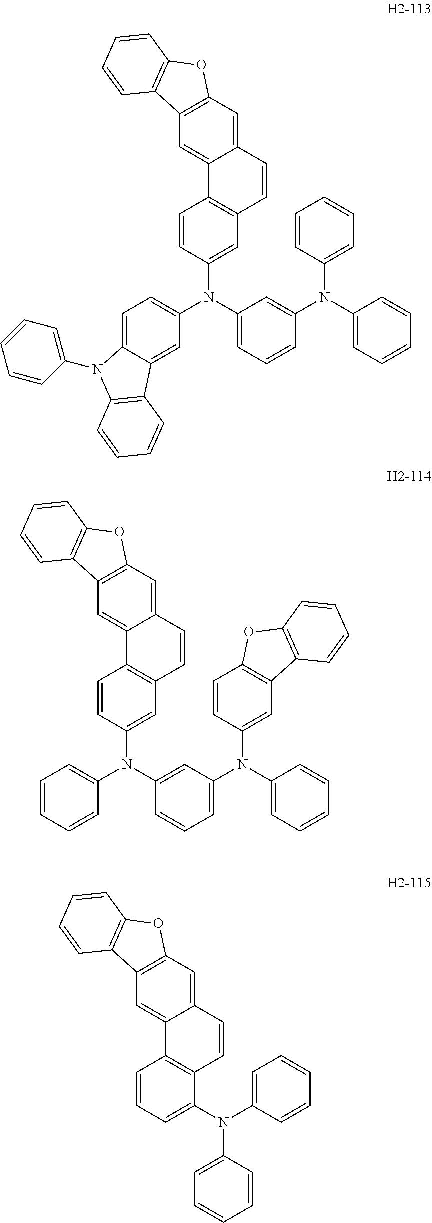 [SMILES: c1ccc(N(c2ccccc2)c2cccc(N(c3ccc4c(ccc5cc6oc7ccccc7c6cc54)c3)c3ccc4c(c3)c3ccccc3n4-c3ccccc3)c2)cc1 ; c1ccc(N(c2cccc(N(c3ccccc3)c3ccc4oc5ccccc5c4c3)c2)c2ccc3c(ccc4cc5oc6ccccc6c5cc43)c2)cc1 ; c1ccc(N(c2ccccc2)c2cccc3c2ccc2cc4oc5ccccc5c4cc23)cc1]

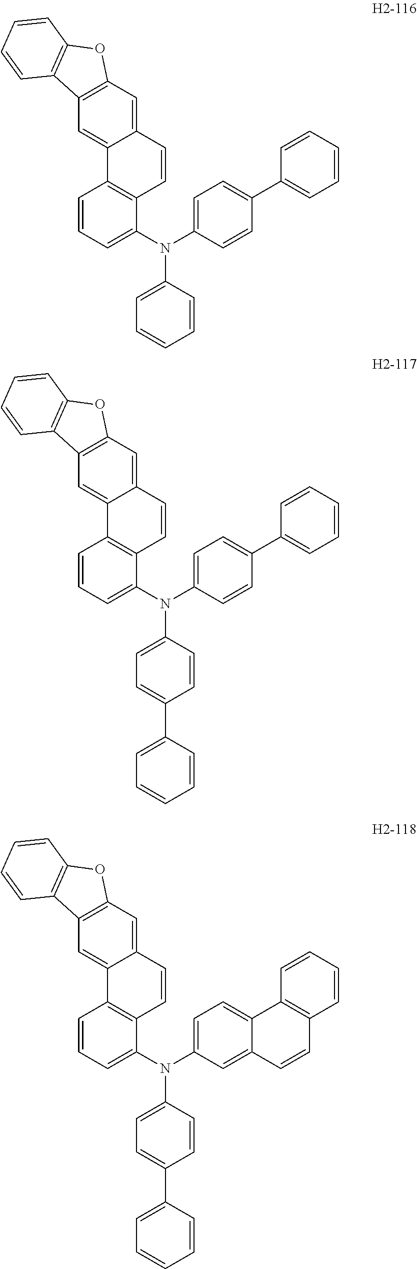 [SMILES: c1ccc(-c2ccc(N(c3ccccc3)c3cccc4c3ccc3cc5oc6ccccc6c5cc34)cc2)cc1 ; c1ccc(-c2ccc(N(c3ccc(-c4ccccc4)cc3)c3cccc4c3ccc3cc5oc6ccccc6c5cc34)cc2)cc1 ; c1ccc(-c2ccc(N(c3ccc4c(ccc5ccccc54)c3)c3cccc4c3ccc3cc5oc6ccccc6c5cc34)cc2)cc1]

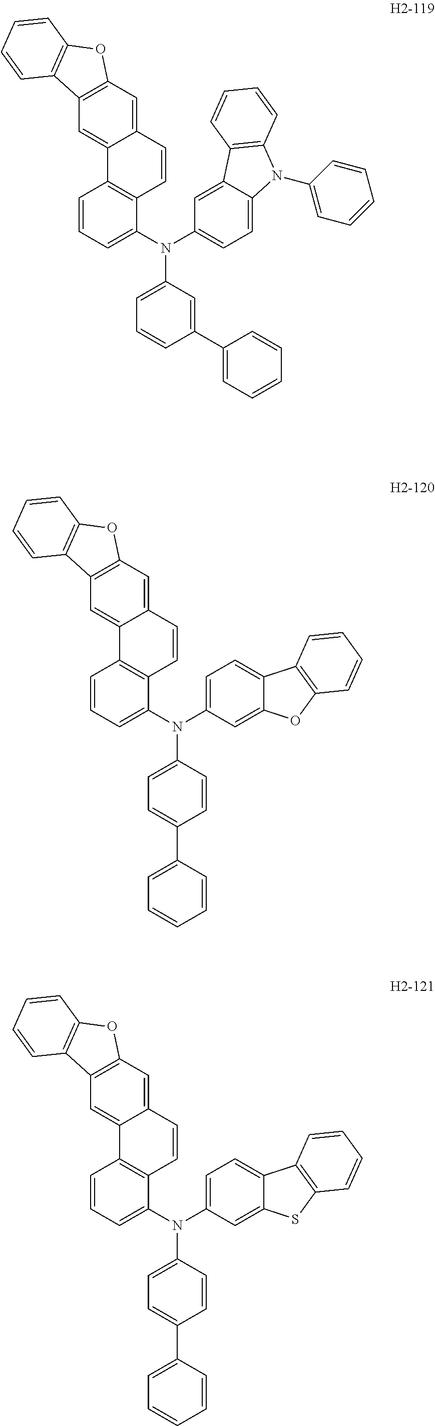 [SMILES: c1ccc(-c2cccc(N(c3ccc4c(c3)c3ccccc3n4-c3ccccc3)c3cccc4c3ccc3cc5oc6ccccc6c5cc34)c2)cc1 ; c1ccc(-c2ccc(N(c3ccc4c(c3)oc3ccccc34)c3cccc4c3ccc3cc5oc6ccccc6c5cc34)cc2)cc1 ; c1ccc(-c2ccc(N(c3ccc4c(c3)sc3ccccc34)c3cccc4c3ccc3cc5oc6ccccc6c5cc34)cc2)cc1]

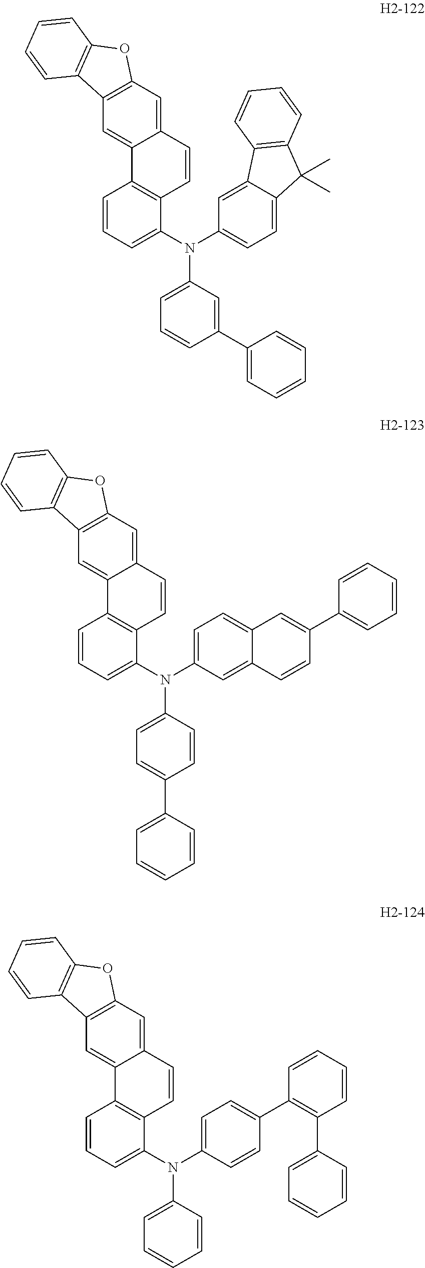 [SMILES: CC1(C)c2ccccc2-c2cc(N(c3cccc(-c4ccccc4)c3)c3cccc4c3ccc3cc5oc6ccccc6c5cc34)ccc21 ; c1ccc(-c2ccc(N(c3ccc4cc(-c5ccccc5)ccc4c3)c3cccc4c3ccc3cc5oc6ccccc6c5cc34)cc2)cc1 ; c1ccc(-c2ccccc2-c2ccc(N(c3ccccc3)c3cccc4c3ccc3cc5oc6ccccc6c5cc34)cc2)cc1]

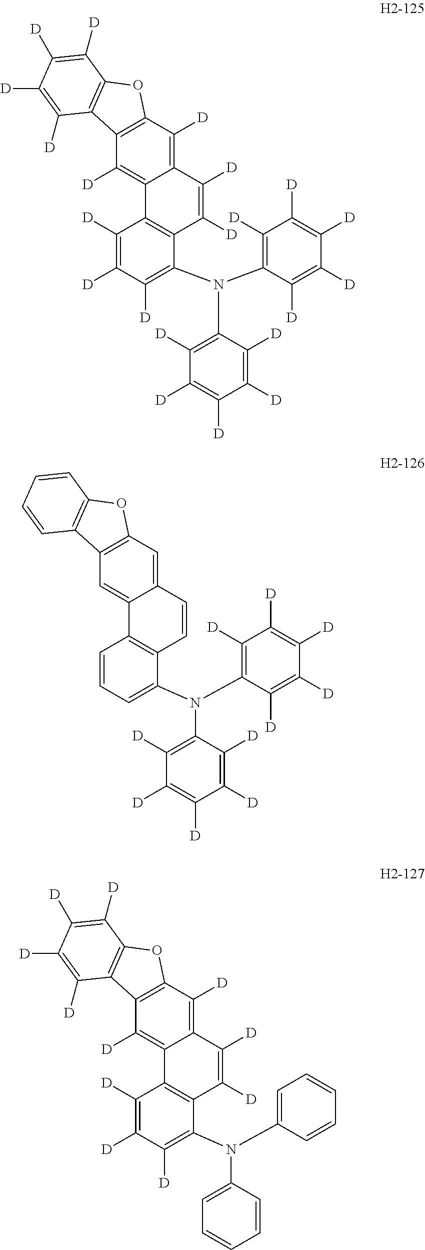 [SMILES: [2H]c1c([2H])c([2H])c(N(c2c([2H])c([2H])c([2H])c([2H])c2[2H])c2c([2H])c([2H])c([2H])c3c2c([2H])c([2H])c2c([2H])c4oc5c([2H])c([2H])c([2H])c([2H])c5c4c([2H])c23)c([2H])c1[2H] ; [2H]c1c([2H])c([2H])c(N(c2c([2H])c([2H])c([2H])c([2H])c2[2H])c2cccc3c2ccc2cc4oc5ccccc5c4cc23)c([2H])c1[2H] ; [2H]c1c([2H])c([2H])c2c(oc3c([2H])c4c([2H])c([2H])c5c(N(c6ccccc6)c6ccccc6)c([2H])c([2H])c([2H])c5c4c([2H])c32)c1[2H]]

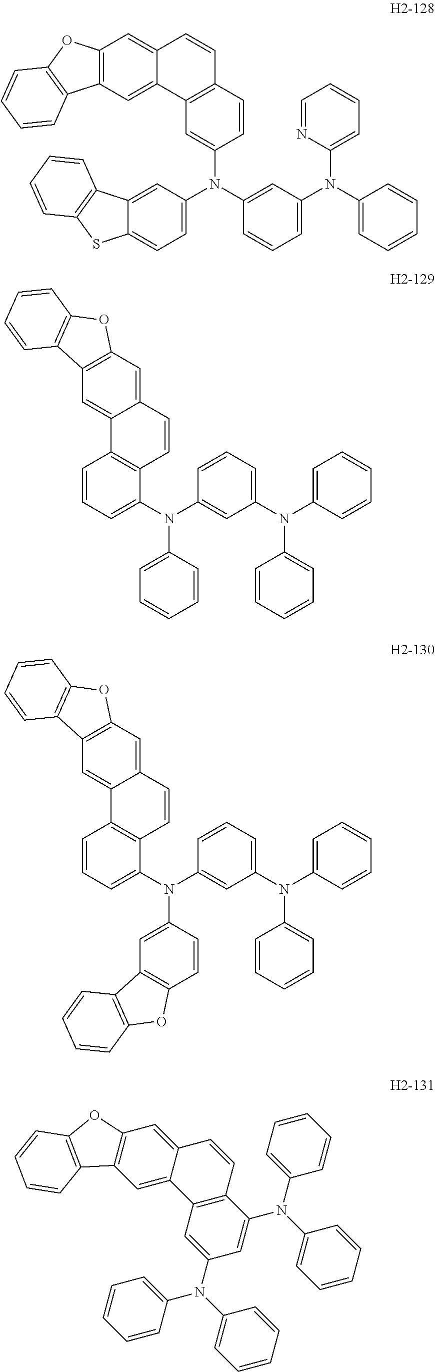 [SMILES: c1ccc(N(c2cccc(N(c3ccc4ccc5cc6oc7ccccc7c6cc5c4c3)c3ccc4sc5ccccc5c4c3)c2)c2ccccn2)cc1 ; c1ccc(N(c2ccccc2)c2cccc(N(c3ccccc3)c3cccc4c3ccc3cc5oc6ccccc6c5cc34)c2)cc1 ; c1ccc(N(c2ccccc2)c2cccc(N(c3ccc4oc5ccccc5c4c3)c3cccc4c3ccc3cc5oc6ccccc6c5cc34)c2)cc1 ; c1ccc(N(c2ccccc2)c2cc(N(c3ccccc3)c3ccccc3)c3ccc4cc5oc6ccccc6c5cc4c3c2)cc1]

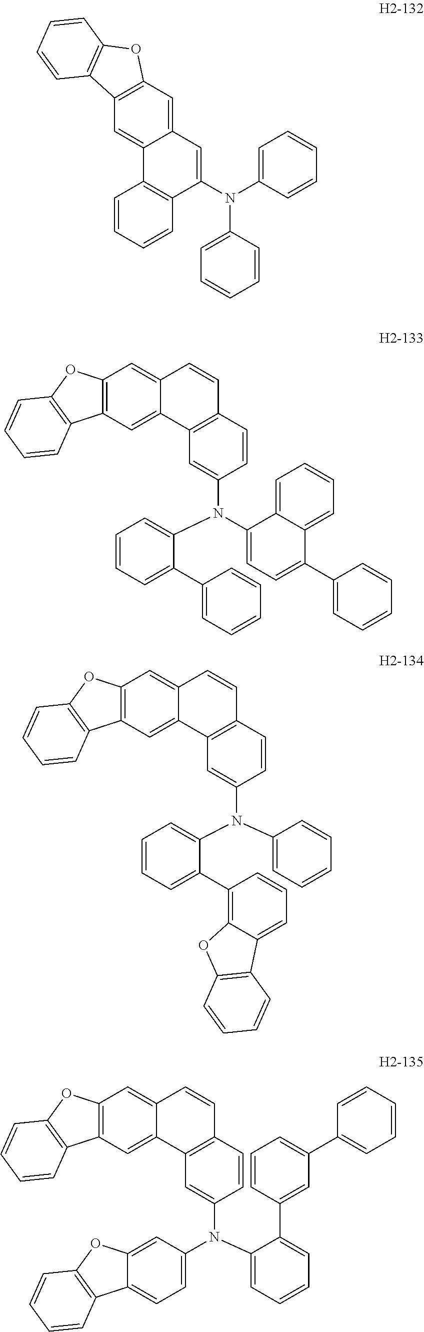

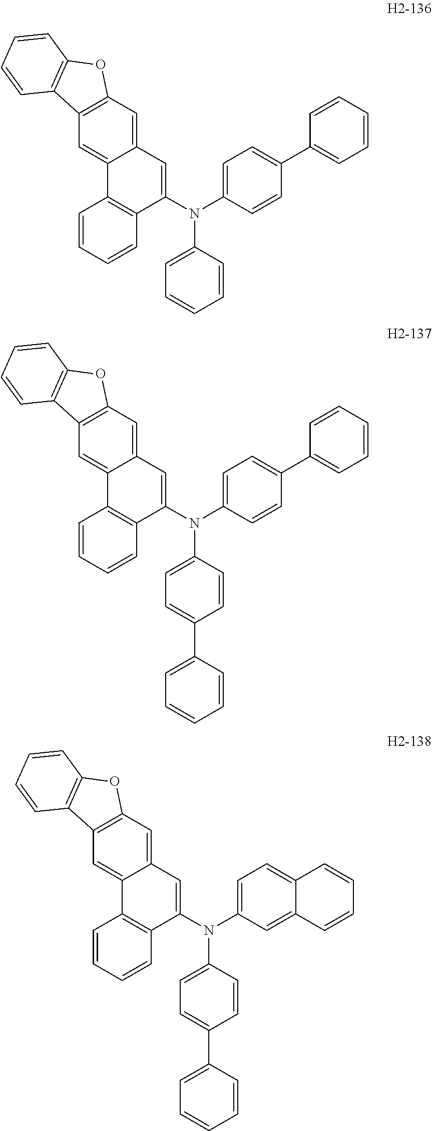 [SMILES: c1ccc(-c2ccc(N(c3ccccc3)c3cc4cc5oc6ccccc6c5cc4c4ccccc34)cc2)cc1 ; c1ccc(-c2ccc(N(c3ccc(-c4ccccc4)cc3)c3cc4cc5oc6ccccc6c5cc4c4ccccc34)cc2)cc1 ; c1ccc(-c2ccc(N(c3ccc4ccccc4c3)c3cc4cc5oc6ccccc6c5cc4c4ccccc34)cc2)cc1]

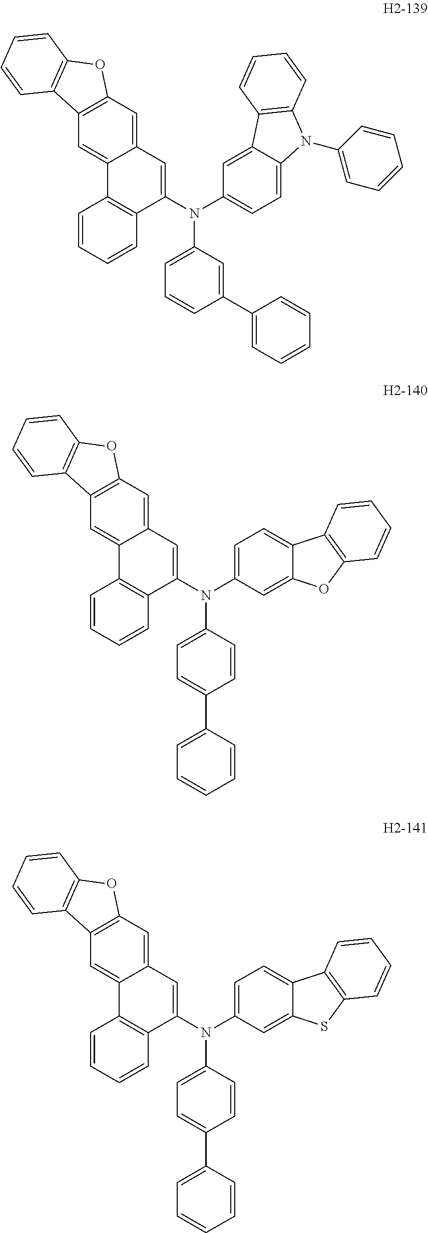 [SMILES: c1ccc(-c2cccc(N(c3ccc4c(c3)c3ccccc3n4-c3ccccc3)c3cc4cc5oc6ccccc6c5cc4c4ccccc34)c2)cc1 ; c1ccc(-c2ccc(N(c3ccc4c(c3)oc3ccccc34)c3cc4cc5oc6ccccc6c5cc4c4ccccc34)cc2)cc1 ; c1ccc(-c2ccc(N(c3ccc4c(c3)sc3ccccc34)c3cc4cc5oc6ccccc6c5cc4c4ccccc34)cc2)cc1]

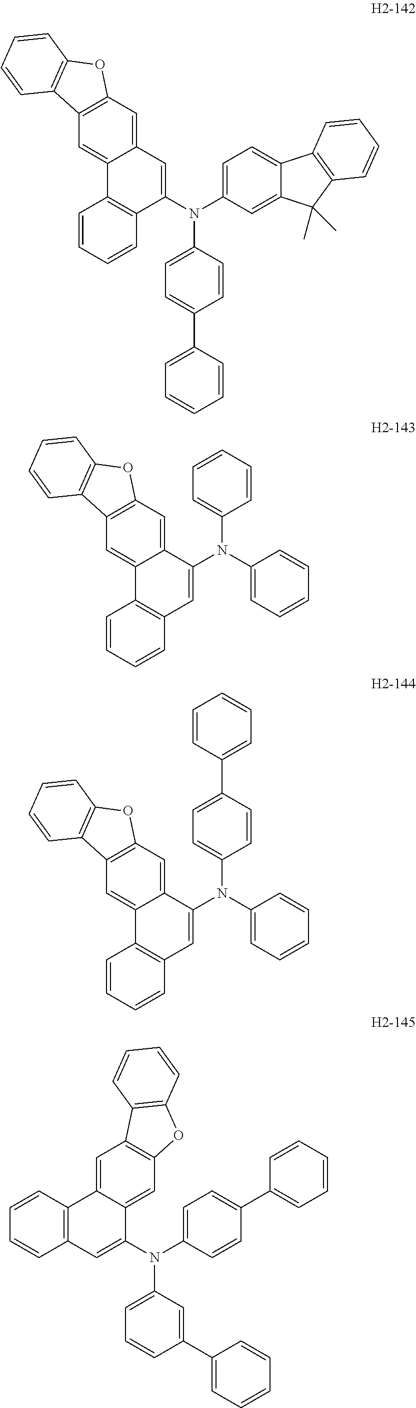

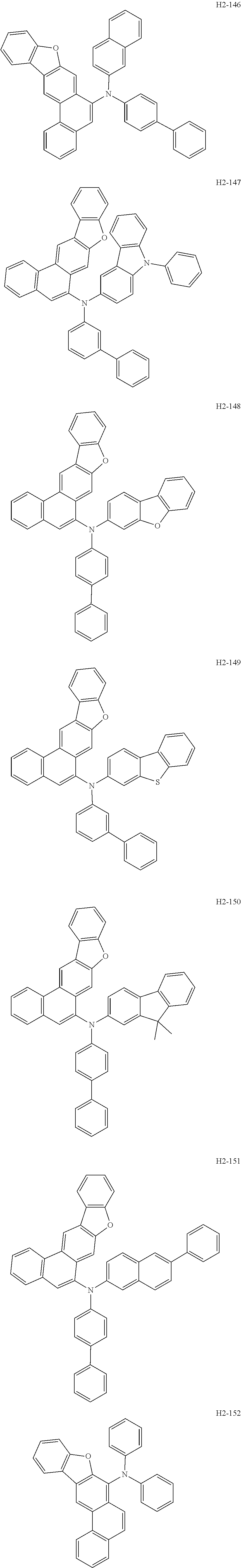

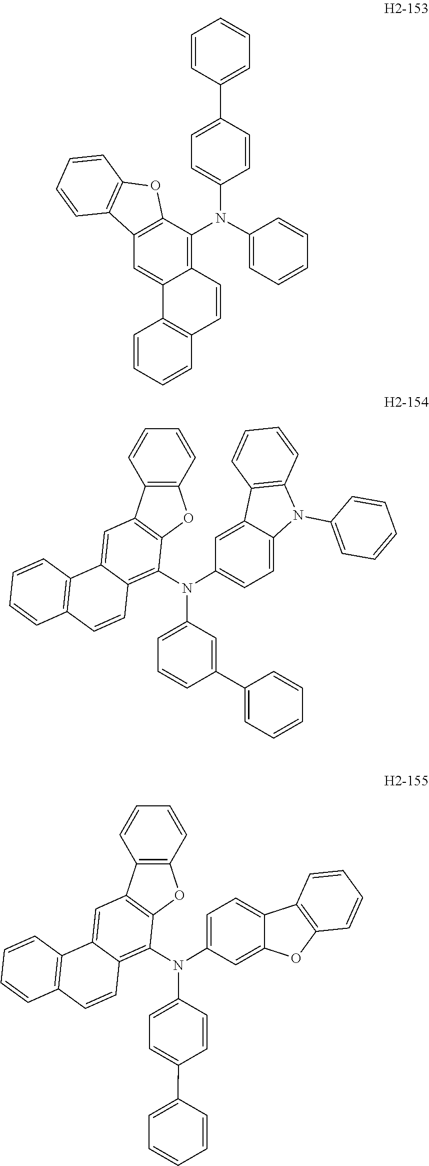

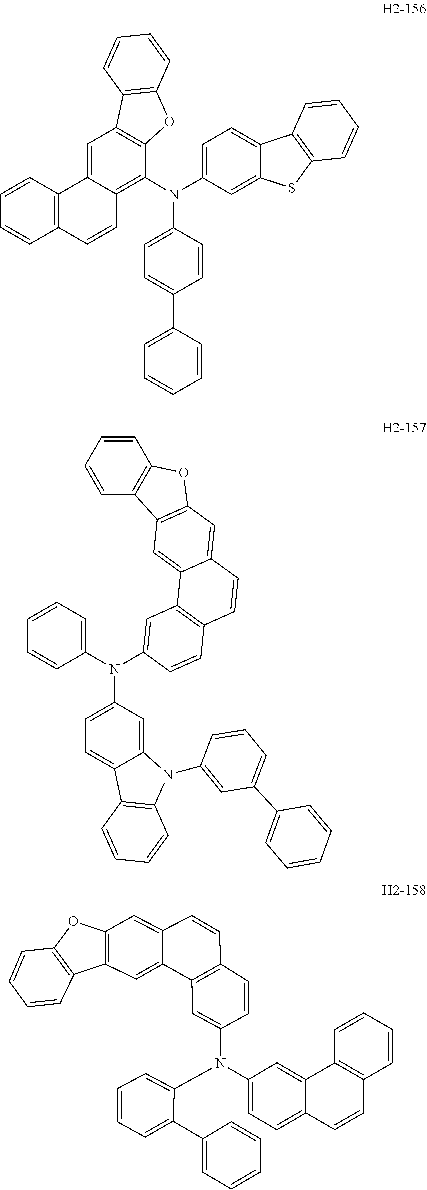 [SMILES: c1ccc(-c2ccc(N(c3ccc4c(c3)sc3ccccc34)c3c4ccc5ccccc5c4cc4c3oc3ccccc34)cc2)cc1 ; c1ccc(-c2cccc(-n3c4ccccc4c4ccc(N(c5ccccc5)c5ccc6ccc7cc8oc9ccccc9c8cc7c6c5)cc43)c2)cc1 ; c1ccc(-c2ccccc2N(c2ccc3ccc4ccccc4c3c2)c2ccc3ccc4cc5oc6ccccc6c5cc4c3c2)cc1]

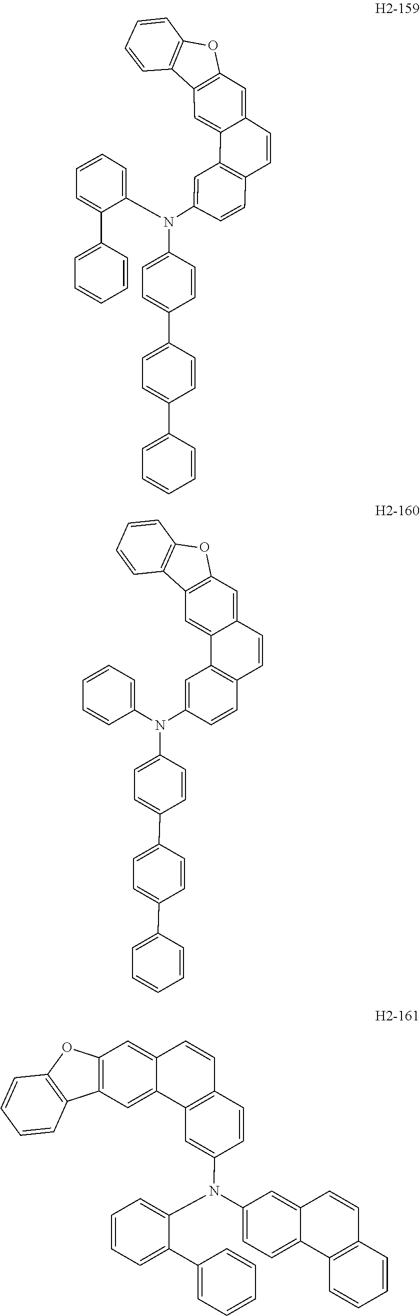

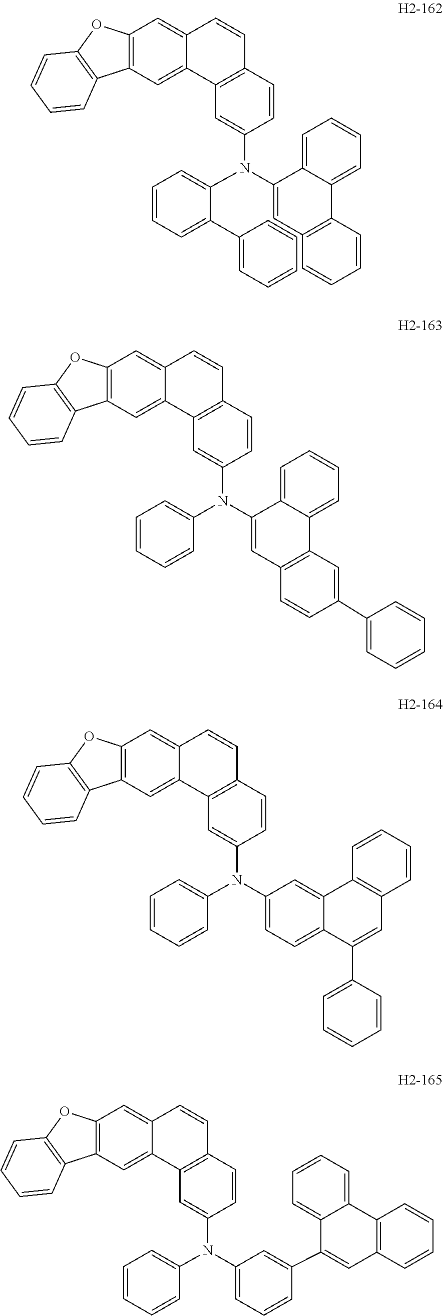 [SMILES: c1ccc(-c2ccccc2N(c2ccc3ccc4cc5oc6ccccc6c5cc4c3c2)c2cc3ccccc3c3ccccc23)cc1 ; c1ccc(-c2ccc3cc(N(c4ccccc4)c4ccc5ccc6cc7oc8ccccc8c7cc6c5c4)c4ccccc4c3c2)cc1 ; c1ccc(-c2cc3ccccc3c3cc(N(c4ccccc4)c4ccc5ccc6cc7oc8ccccc8c7cc6c5c4)ccc23)cc1 ; c1ccc(N(c2cccc(-c3cc4ccccc4c4ccccc34)c2)c2ccc3ccc4cc5oc6ccccc6c5cc4c3c2)cc1]

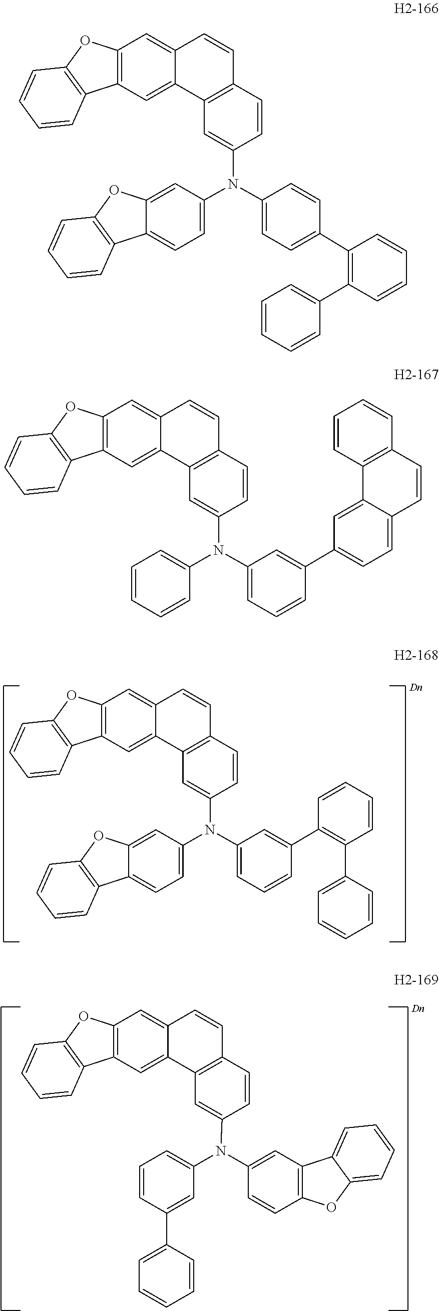

In the compounds above, Dₙ means that n number of hydrogens are replaced with deuterium, and n represents an integer from 1 to the maximum number of hydrogens in the compound.

The compounds represented by Formula 6 according to the present disclosure may be produced by synthetic methods known to one skilled in the art, and in particular, a synthesis method disclosed in various patent documents may be employed. For example, the compounds represented by Formula 6-1 according to the present disclosure may be synthesized by referring to Korean Patent Application Laid-Open Nos. 2017-0022865 (Mar. 2, 2017) and 2018-0099487 (Sep. 5, 2018), but are not limited thereto. For example, the compounds represented by Formula 6-2 may be produced by referring to the following Reaction Scheme 1, but are not limited thereto, and may be produced by synthetic methods known to one skilled in the art.

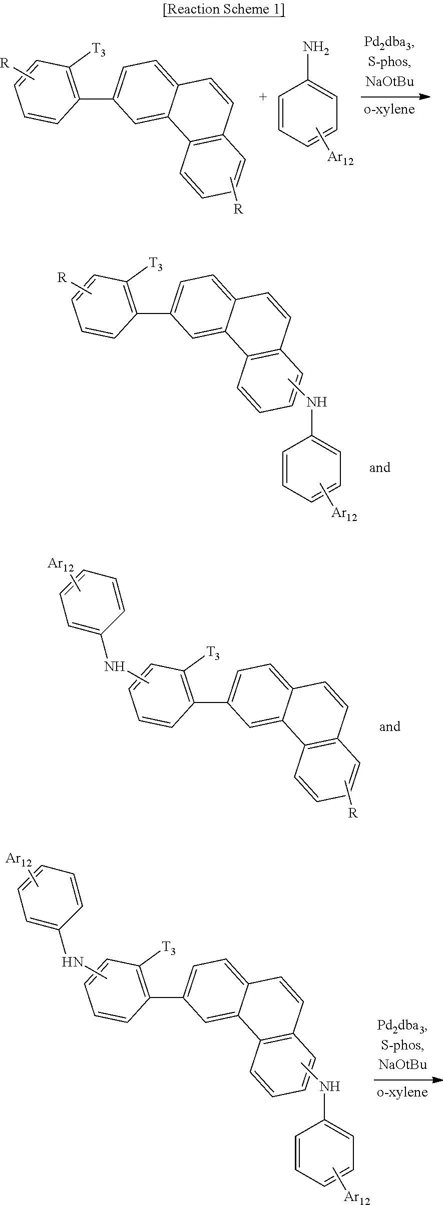

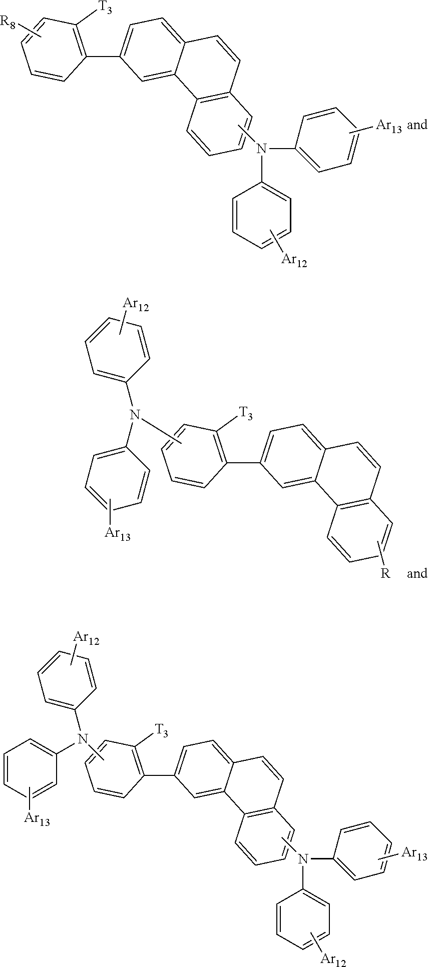

In Reaction Scheme 1, Ar₁₂ and Ar₁₃ are as defined in Formula 6, T₃ is as defined in Formula 6-2, and R is as defined for R₂₂ to R₃₃ in Formula 6-2.

The light-emitting layer of the organic electroluminescent device comprises not only a compound represented by Formula 6, but also a compound represented by Formula 7.

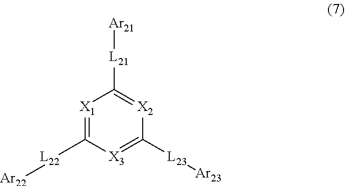

In Formula 7, X₁ to X₃ each independently represent N or CR₂₁; with the proviso that at least one of X₁ to X₃ is N. For example, X₁ to X₃ may each independently be N.

In Formula 7, each R₂₁ independently represents hydrogen or deuterium.

In Formula 7, L₂₁ to L₂₃ each independently represent a single bond, a substituted or unsubstituted (C6-C30)arylene, or a substituted or unsubstituted (3- to 30-membered)heteroarylene. According to one embodiment of the present disclosure, L₂₁ to L₂₃ each independently represent a single bond, a substituted or unsubstituted (C6-C25)arylene, or a substituted or unsubstituted (3- to 25-membered)heteroarylene. According to another embodiment of the present disclosure, L₂₁ to L₂₃ each independently represent a single bond, a substituted or unsubstituted (C6-C20)arylene, or a substituted or unsubstituted (3- to 20-membered)heteroarylene. For example, L₂₁ to L₂₃ may each independently be a single bond, a phenylene unsubstituted or substituted with deuterium, a biphenylene unsubstituted or substituted with deuterium, a terphenylene, a naphthylene unsubstituted or substituted with deuterium, a phenanthrenylene, a dibenzofuranylene, or a dibenzothiophenylene, and these may be further substituted with deuterium.

In Formula 7, Ar₂₁ to Ar₂₃ each independently represent a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl, with the proviso that at least one of Ar₂₁ to Ar₂₃ is a substituted or unsubstituted (3- to 30-membered)heteroaryl. According to one embodiment of the present disclosure, Ar₂₁ to Ar₂₃ each independently represent a substituted or unsubstituted (C6-C30)aryl, or a substituted or unsubstituted (3- to 30-membered)heteroaryl. For example, Ar₂₁ to Ar₂₃ may each independently be a phenyl unsubstituted or substituted with deuterium, a naphthyl unsubstituted or substituted with a naphthyl unsubstituted or substituted with deuterium, or a phenyl, a phenanthrenyl, or a dibenzofuranyl; a biphenyl unsubstituted or substituted with deuterium, a naphthyl, or a dibenzofuranyl; a triphenylenyl; a triphenylsilyl; an o-terphenyl; a m-terphenyl; a p-terphenyl unsubstituted or substituted with deuterium; a phenanthrenyl unsubstituted or substituted with a phenyl or a naphthyl; a benzophenanthrenyl unsubstituted or substituted with deuterium; a naphthyl unsubstituted or substituted with deuterium, a phenyl unsubstituted or substituted with deuterium, a naphthyl unsubstituted or substituted with deuterium, a biphenyl unsubstituted or substituted with deuterium, a dibenzofuranyl, or a dibenzothiophenyl; a quarterphenyl; a fluoranthenyl; a dibenzofuranly unsubstituted or substituted with deuterium, a phenyl, a biphenyl, a naphthyl, a phenanthrenyl, or a triphenylenyl; or a dibenzothiophenyl unsubstituted or substituted with a phenyl, and these may be further substituted with deuterium.

According to one embodiment of the present disclosure, Formula 7 may be represented by any one of the following Formulas 7-1 to 7-4.

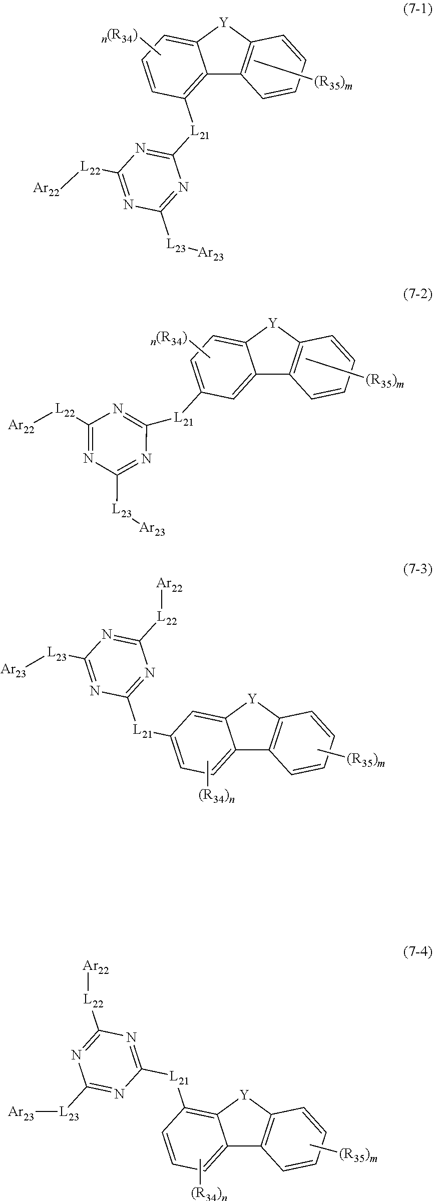

In Formulas 7-1 to 7-4, Y represents O, S, or NR₃₆. For example, Y is O or S.

In Formulas 7-1 to 7-4, R₃₄ to R₃₆ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino; or may be linked to an adjacent substituent(s) to form a ring(s). According to one embodiment of the present disclosure, R₃₄ to R₃₆ each independently represent hydrogen, deuterium, or a substituted or unsubstituted (C6-C30)aryl. According to another embodiment of the present disclosure, R₃₄ to R₃₆ each independently represent hydrogen, deuterium, or a substituted or unsubstituted (C6-C20)aryl. For example, R₃₄ to R₃₆ may each independently be hydrogen; deuterium; a phenyl unsubstituted or substituted with a naphthyl; a naphthyl unsubstituted or substituted with a phenyl; a biphenyl; or a phenanthrenyl, and these may be further substituted with deuterium.

In Formulas 7-1 to 7-4, L₂₁ to L₂₃, Ar₂₂, and Ar₂₃ are as defined in Formula 7.

In Formulas 7-1 to 7-4, n represents an integer of 1 to 3, and m represents an integer of 1 to 4; if n and m are integers of 2 or more, each R₃₄ and each R₃₅ may be the same as or different from each other.

The organic electroluminescent compound represented by Formula 7 may be selected from the following compounds, but is not limited thereto.

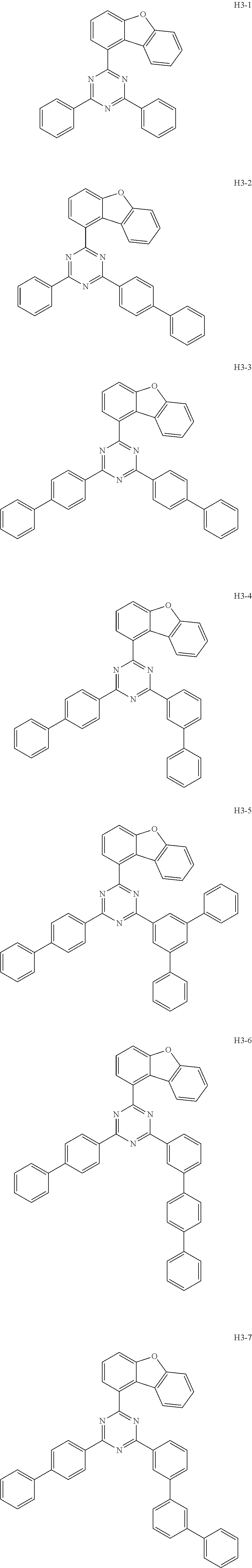

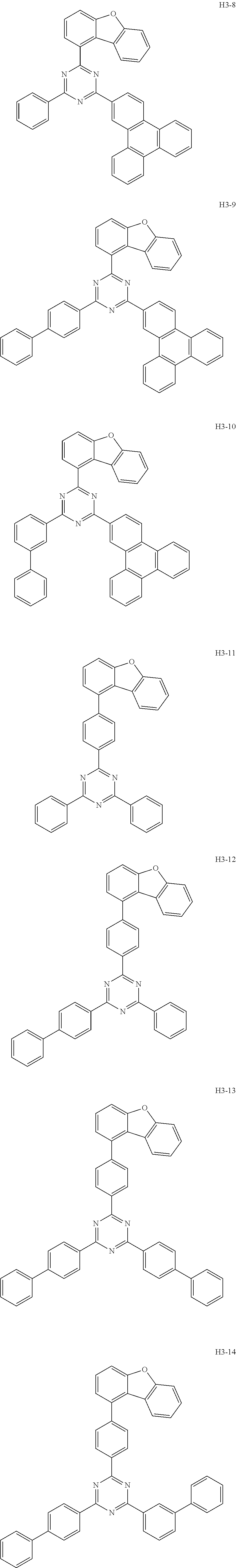

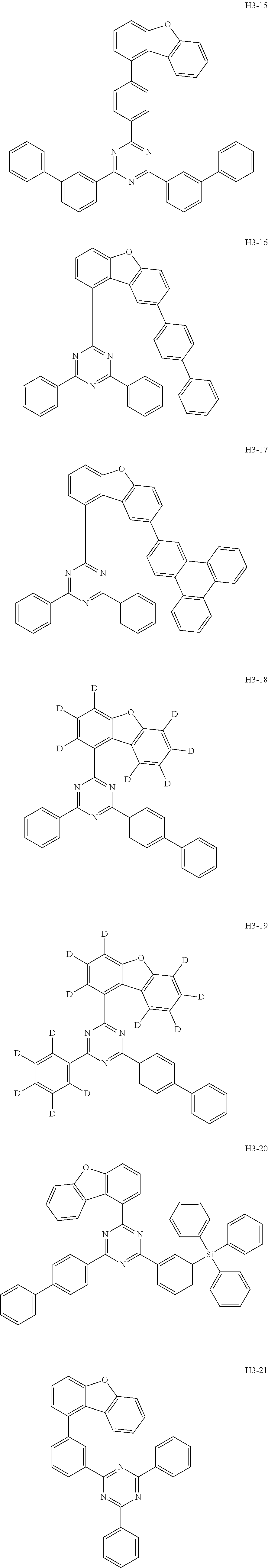

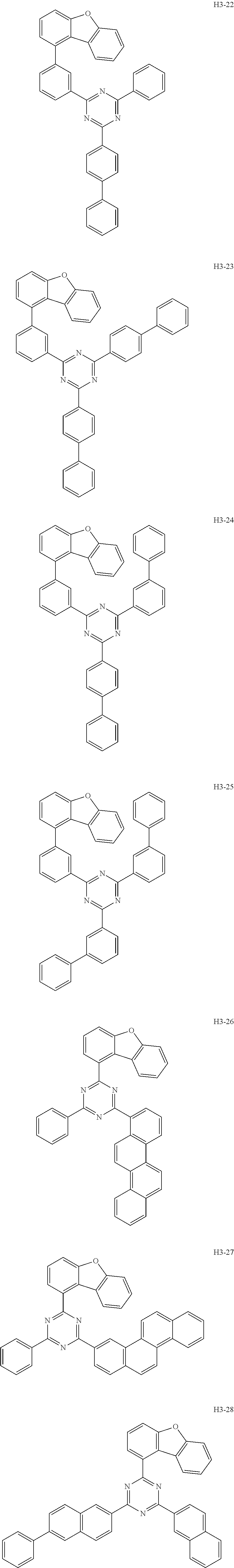

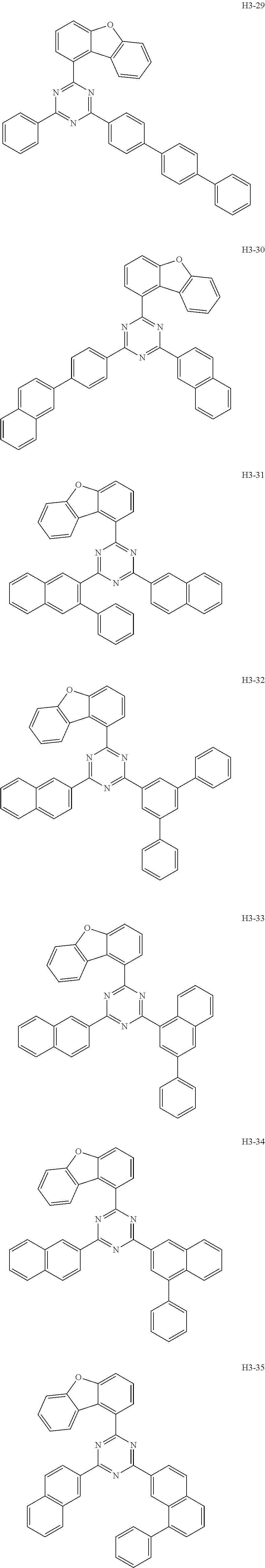

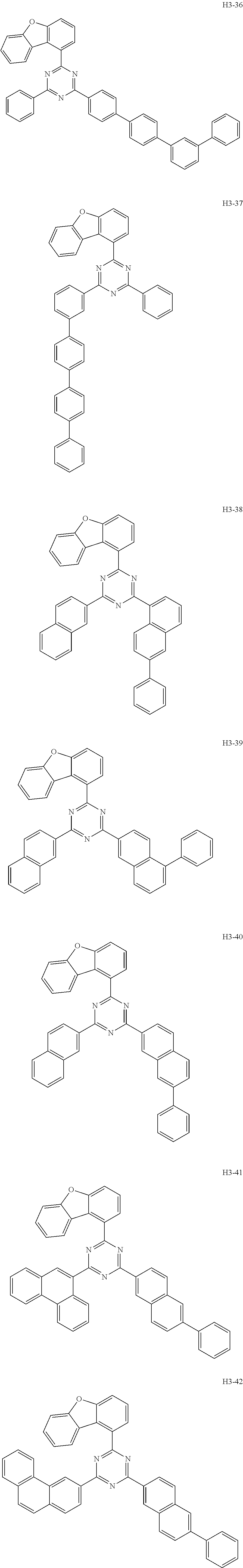

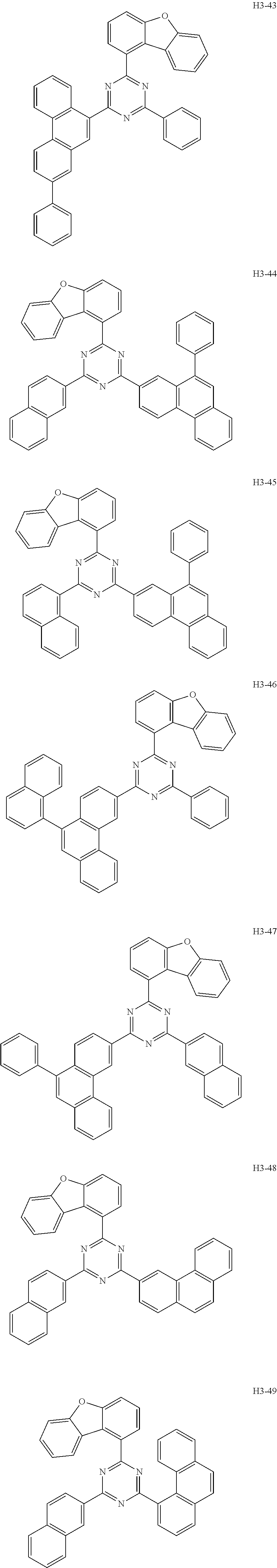

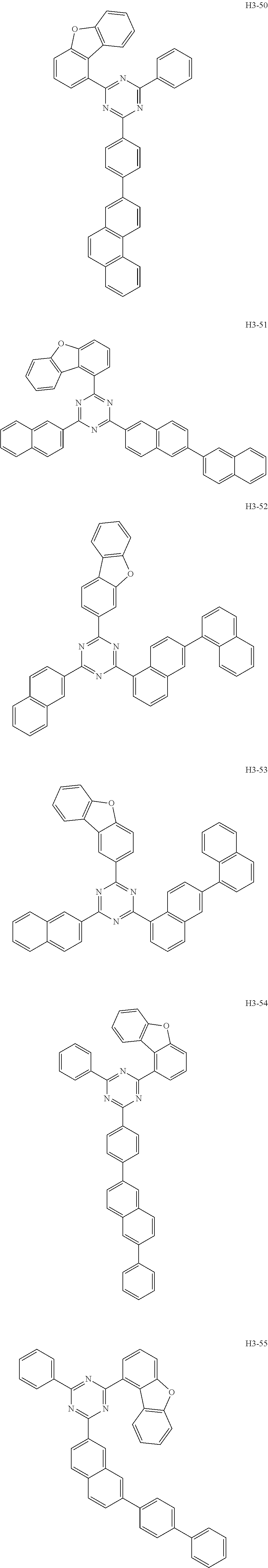 [SMILES: c1ccc(-c2nc(-c3ccc(-c4ccc5c(ccc6ccccc65)c4)cc3)nc(-c3cccc4oc5ccccc5c34)n2)cc1 ; c1ccc2cc(-c3ccc4cc(-c5nc(-c6ccc7ccccc7c6)nc(-c6cccc7oc8ccccc8c67)n5)ccc4c3)ccc2c1 ; c1ccc2cc(-c3nc(-c4ccc5c(c4)oc4ccccc45)nc(-c4cccc5cc(-c6cccc7ccccc67)ccc45)n3)ccc2c1 ; c1ccc2cc(-c3nc(-c4ccc5oc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6cccc7oc8ccccc8c67)n5)cc4)ccc3c2)cc1 ; c1ccc(-c2ccc(-c3ccc4ccc(-c5nc(-c6ccccc6)nc(-c6cccc7oc8ccccc8c67)n5)cc4c3)cc2)cc1]

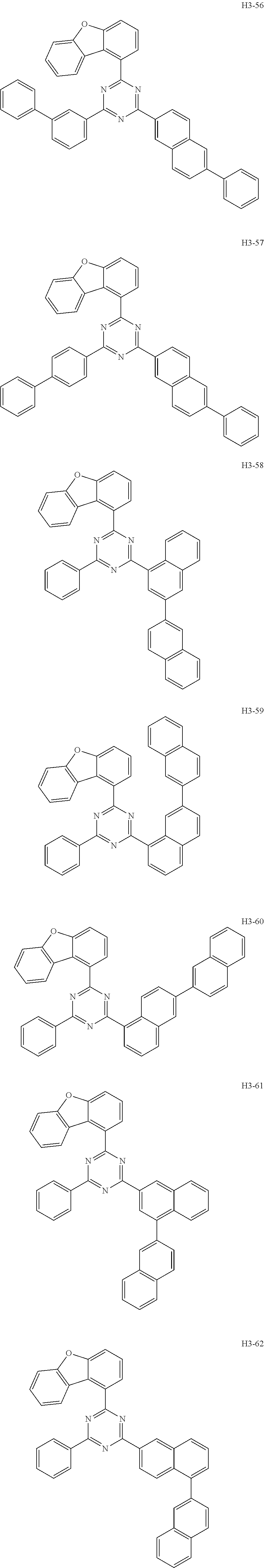

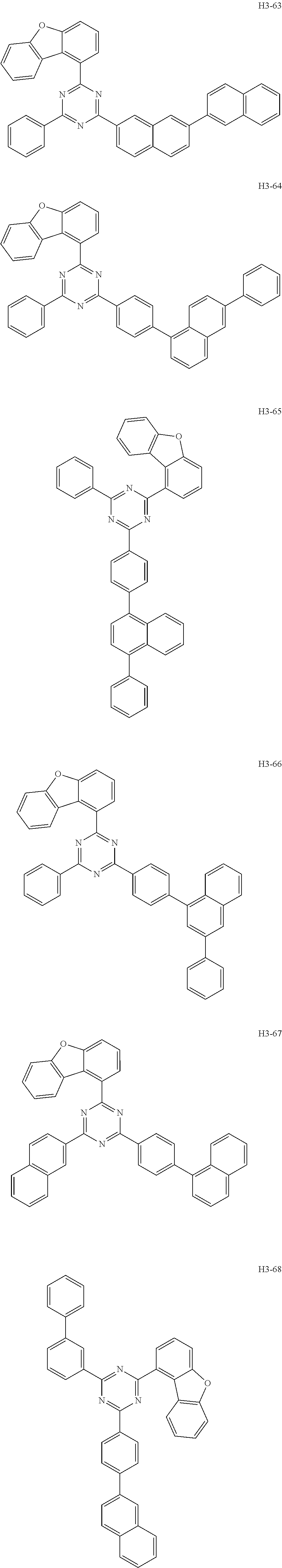 [SMILES: c1ccc(-c2nc(-c3ccc4ccc(-c5ccc6ccccc6c5)cc4c3)nc(-c3cccc4oc5ccccc5c34)n2)cc1 ; c1ccc(-c2ccc3c(-c4ccc(-c5nc(-c6ccccc6)nc(-c6cccc7oc8ccccc8c67)n5)cc4)cccc3c2)cc1 ; c1ccc(-c2nc(-c3ccc(-c4ccc(-c5ccccc5)c5ccccc45)cc3)nc(-c3cccc4oc5ccccc5c34)n2)cc1 ; c1ccc(-c2cc(-c3ccc(-c4nc(-c5ccccc5)nc(-c5cccc6oc7ccccc7c56)n4)cc3)c3ccccc3c2)cc1 ; c1ccc2cc(-c3nc(-c4ccc(-c5cccc6ccccc56)cc4)nc(-c4cccc5oc6ccccc6c45)n3)ccc2c1 ; c1ccc(-c2cccc(-c3nc(-c4ccc(-c5ccc6ccccc6c5)cc4)nc(-c4cccc5oc6ccccc6c45)n3)c2)cc1]

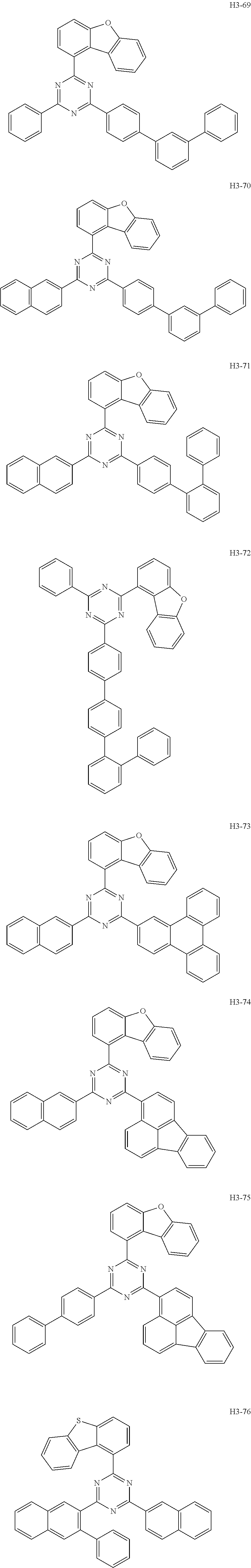

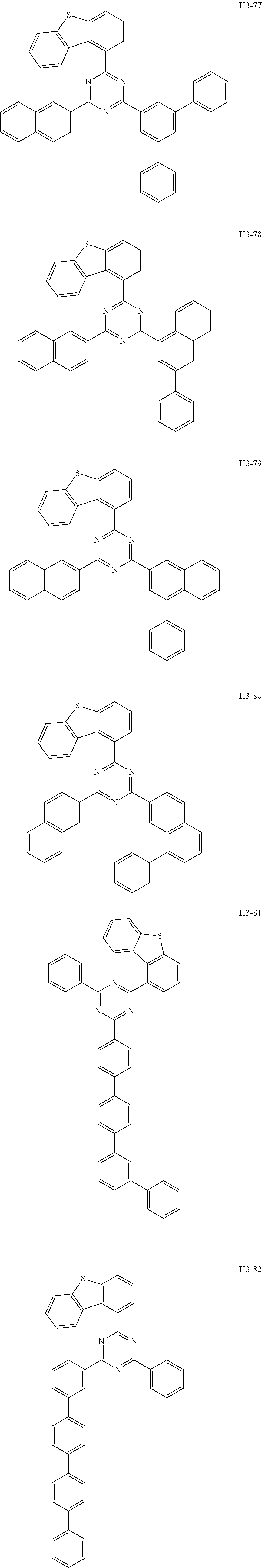

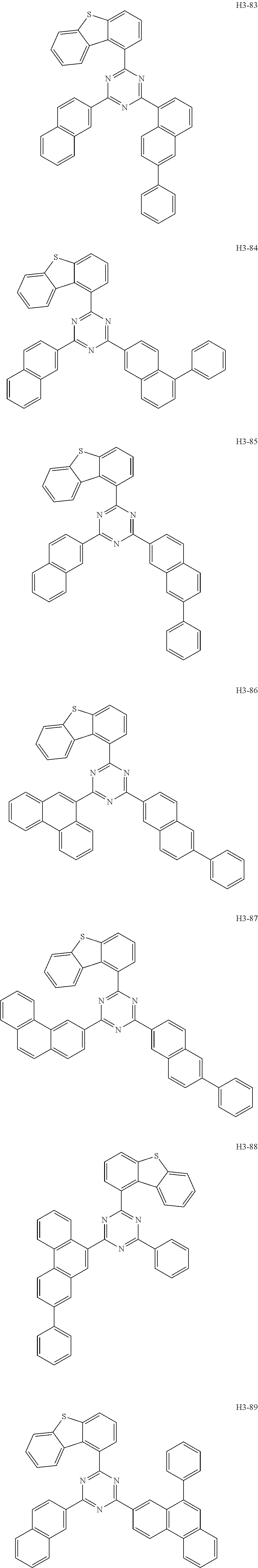

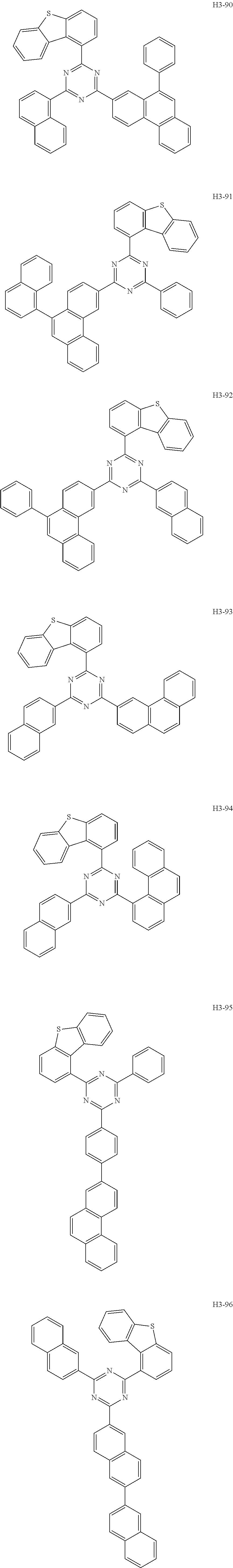

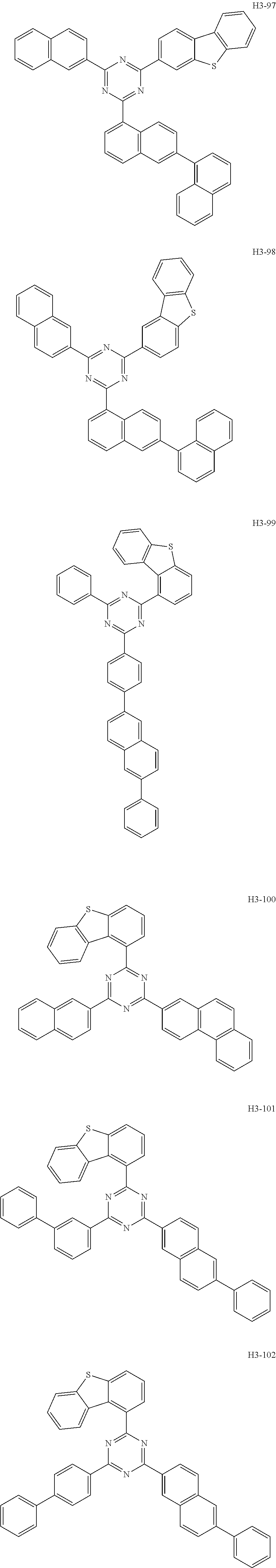 [SMILES: c1ccc2cc(-c3nc(-c4ccc5c(c4)sc4ccccc45)nc(-c4cccc5cc(-c6cccc7ccccc67)ccc45)n3)ccc2c1 ; c1ccc2cc(-c3nc(-c4ccc5sc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6cccc7sc8ccccc8c67)n5)cc4)ccc3c2)cc1 ; c1ccc2cc(-c3nc(-c4ccc5c(ccc6ccccc65)c4)nc(-c4cccc5sc6ccccc6c45)n3)ccc2c1 ; c1ccc(-c2cccc(-c3nc(-c4ccc5cc(-c6ccccc6)ccc5c4)nc(-c4cccc5sc6ccccc6c45)n3)c2)cc1 ; c1ccc(-c2ccc(-c3nc(-c4ccc5cc(-c6ccccc6)ccc5c4)nc(-c4cccc5sc6ccccc6c45)n3)cc2)cc1]

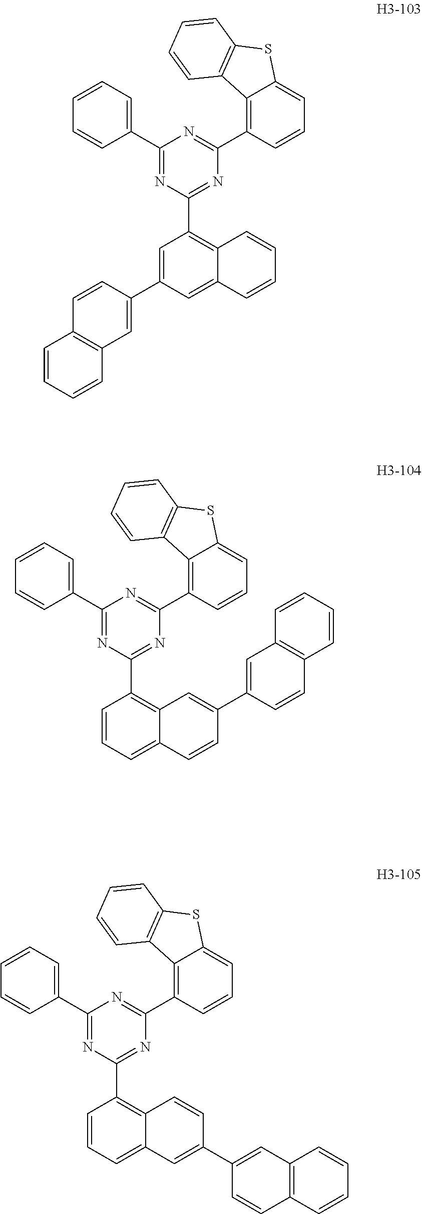 [SMILES: c1ccc(-c2nc(-c3cc(-c4ccc5ccccc5c4)cc4ccccc34)nc(-c3cccc4sc5ccccc5c34)n2)cc1 ; c1ccc(-c2nc(-c3cccc4ccc(-c5ccc6ccccc6c5)cc34)nc(-c3cccc4sc5ccccc5c34)n2)cc1 ; c1ccc(-c2nc(-c3cccc4cc(-c5ccc6ccccc6c5)ccc34)nc(-c3cccc4sc5ccccc5c34)n2)cc1]

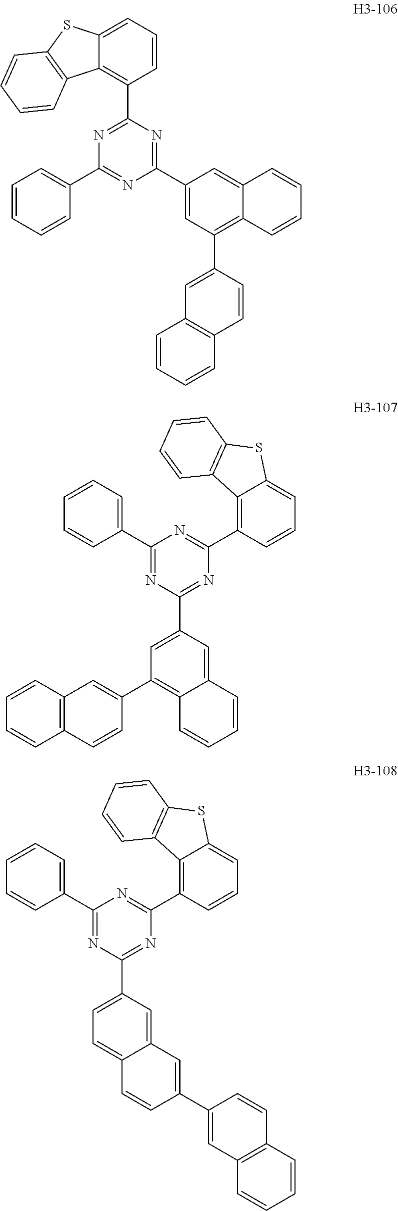

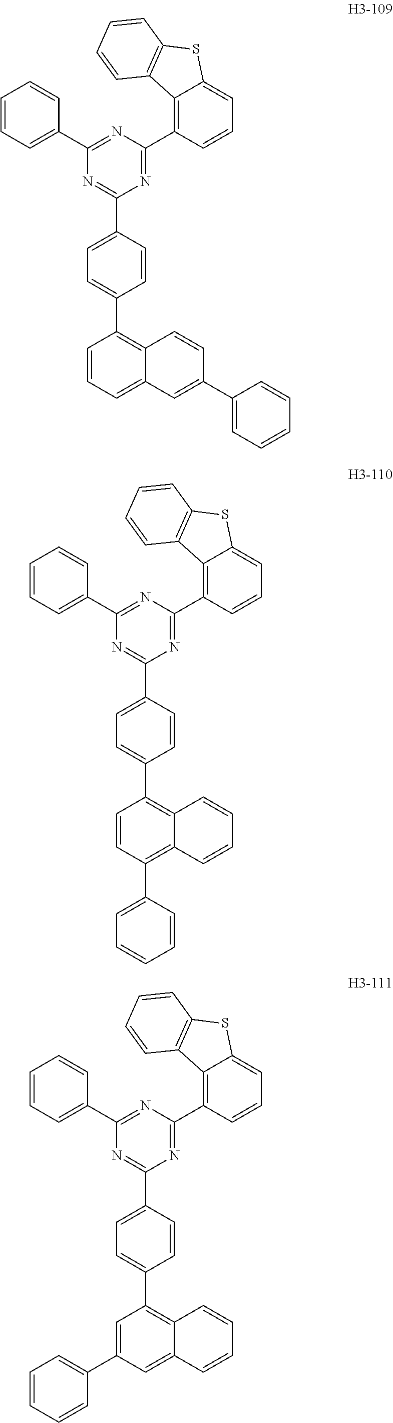

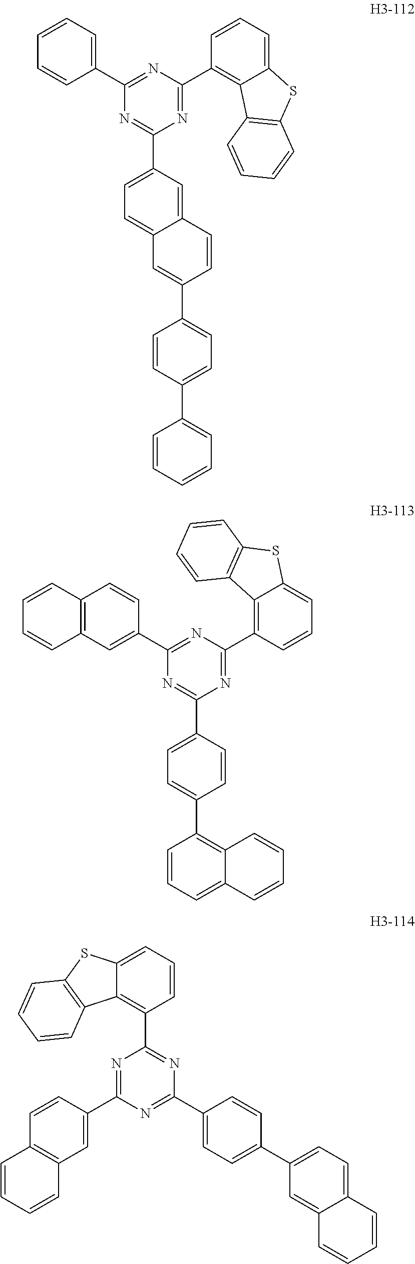

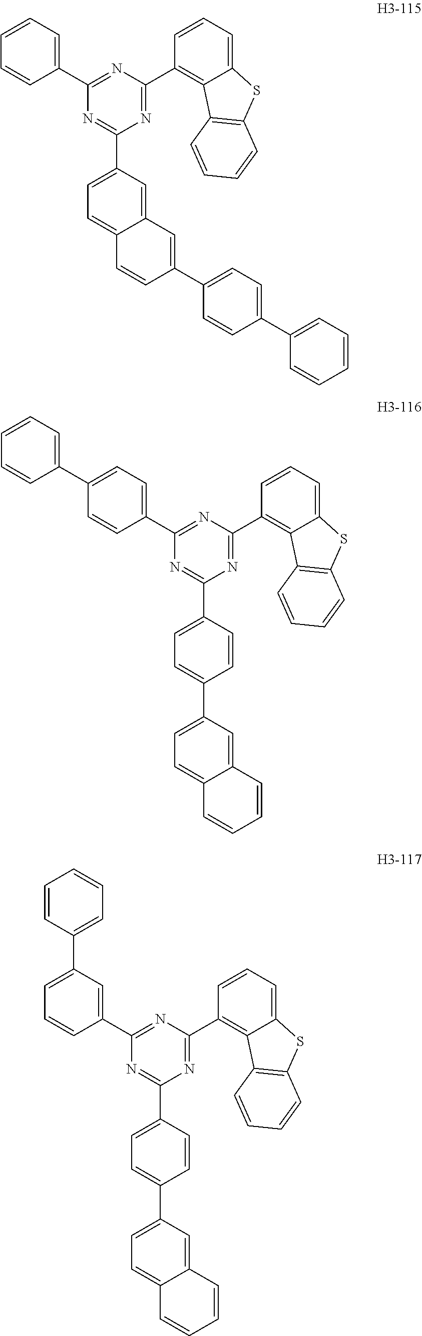 [SMILES: c1ccc(-c2ccc(-c3ccc4ccc(-c5nc(-c6ccccc6)nc(-c6cccc7sc8ccccc8c67)n5)cc4c3)cc2)cc1 ; c1ccc(-c2ccc(-c3nc(-c4ccc(-c5ccc6ccccc6c5)cc4)nc(-c4cccc5sc6ccccc6c45)n3)cc2)cc1 ; c1ccc(-c2cccc(-c3nc(-c4ccc(-c5ccc6ccccc6c5)cc4)nc(-c4cccc5sc6ccccc6c45)n3)c2)cc1]

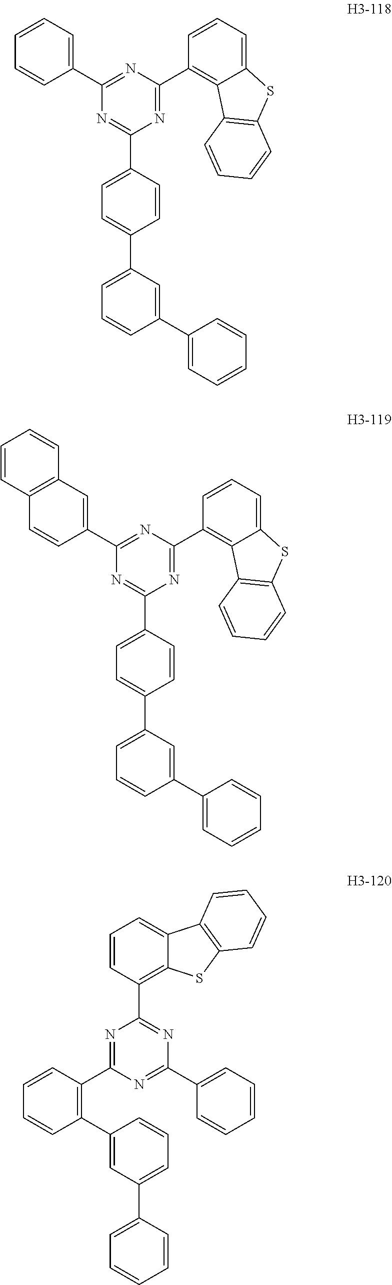

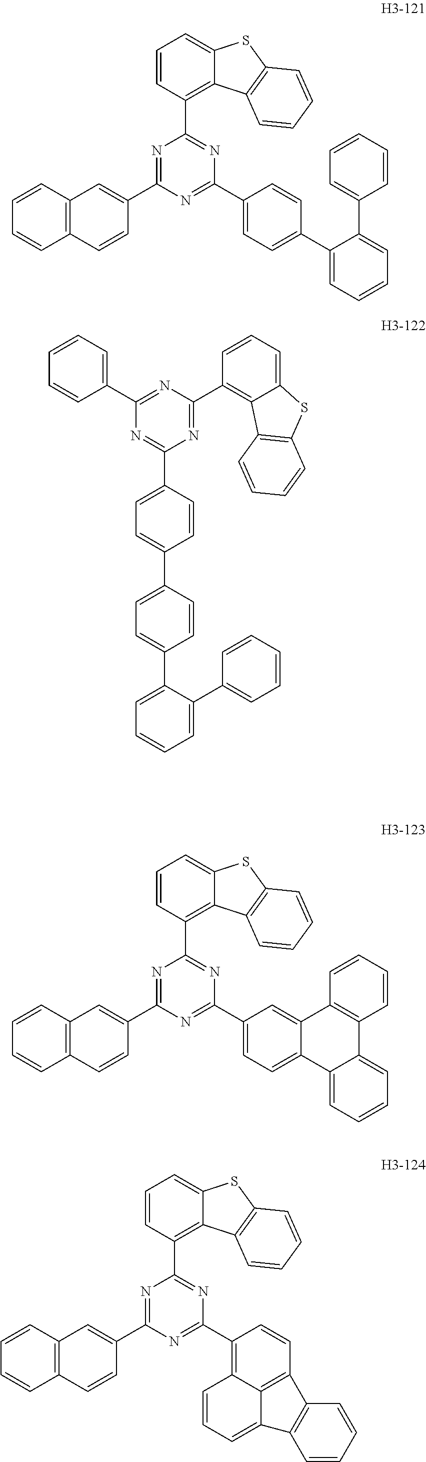 [SMILES: c1ccc(-c2ccccc2-c2ccc(-c3nc(-c4ccc5ccccc5c4)nc(-c4cccc5sc6ccccc6c45)n3)cc2)cc1 ; c1ccc(-c2nc(-c3ccc(-c4ccc(-c5ccccc5-c5ccccc5)cc4)cc3)nc(-c3cccc4sc5ccccc5c34)n2)cc1 ; c1ccc2cc(-c3nc(-c4ccc5c6ccccc6c6ccccc6c5c4)nc(-c4cccc5sc6ccccc6c45)n3)ccc2c1 ; c1ccc2c(c1)-c1cccc3c(-c4nc(-c5ccc6ccccc6c5)nc(-c5cccc6sc7ccccc7c56)n4)ccc-2c13]

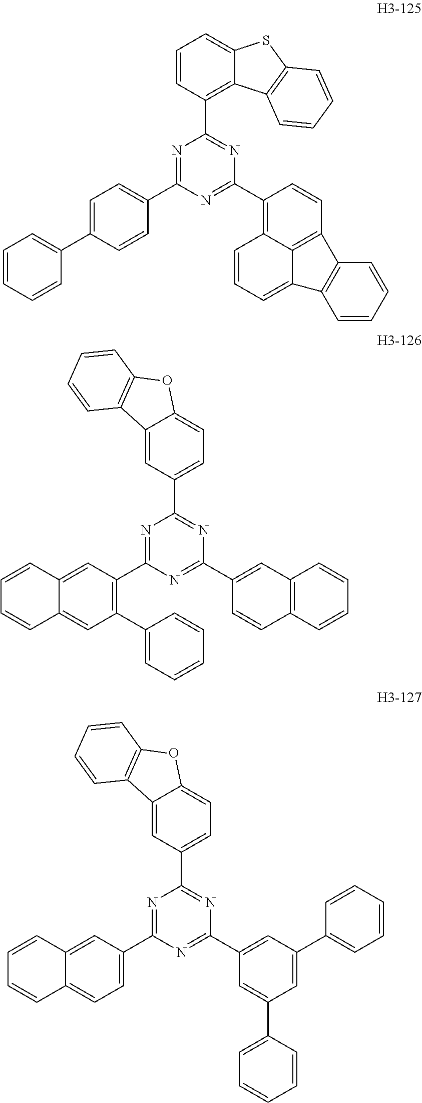 [SMILES: c1ccc(-c2ccc(-c3nc(-c4ccc5c6c(cccc46)-c4ccccc4-5)nc(-c4cccc5sc6ccccc6c45)n3)cc2)cc1 ; c1ccc(-c2cc3ccccc3cc2-c2nc(-c3ccc4ccccc4c3)nc(-c3ccc4oc5ccccc5c4c3)n2)cc1 ; c1ccc(-c2cc(-c3ccccc3)cc(-c3nc(-c4ccc5ccccc5c4)nc(-c4ccc5oc6ccccc6c5c4)n3)c2)cc1]

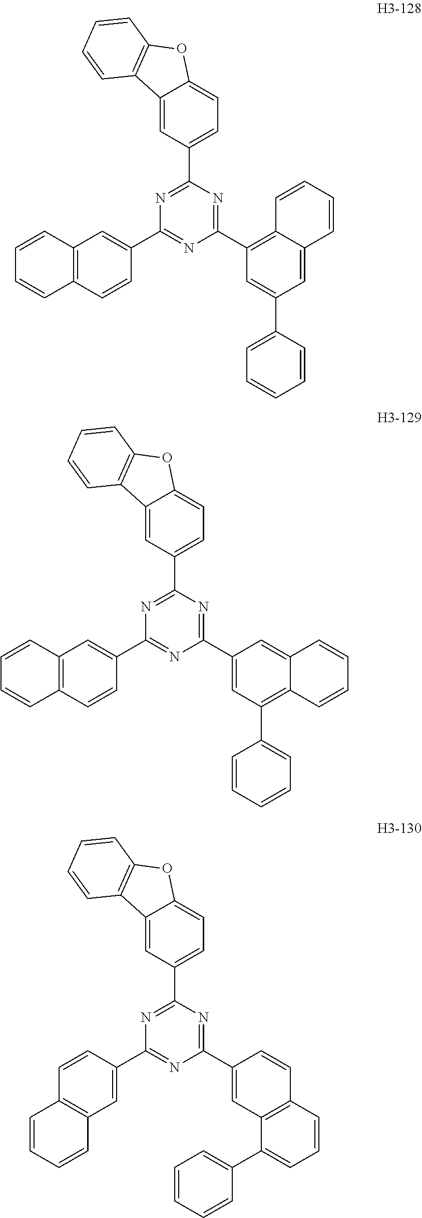 [SMILES: c1ccc(-c2cc(-c3nc(-c4ccc5ccccc5c4)nc(-c4ccc5oc6ccccc6c5c4)n3)c3ccccc3c2)cc1 ; c1ccc(-c2cc(-c3nc(-c4ccc5ccccc5c4)nc(-c4ccc5oc6ccccc6c5c4)n3)cc3ccccc23)cc1 ; c1ccc(-c2cccc3ccc(-c4nc(-c5ccc6ccccc6c5)nc(-c5ccc6oc7ccccc7c6c5)n4)cc23)cc1]

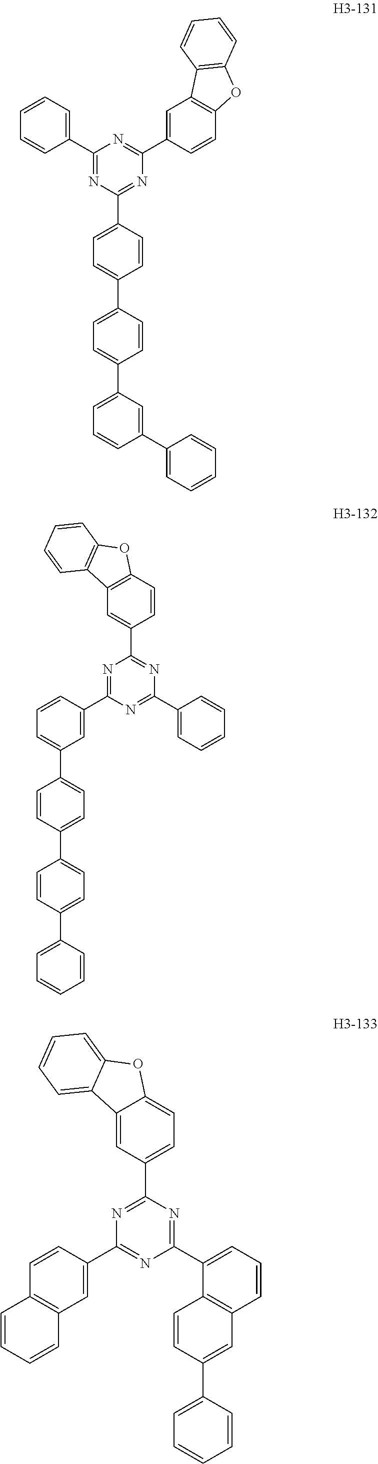 [SMILES: c1ccc(-c2cccc(-c3ccc(-c4ccc(-c5nc(-c6ccccc6)nc(-c6ccc7oc8ccccc8c7c6)n5)cc4)cc3)c2)cc1 ; c1ccc(-c2ccc(-c3ccc(-c4cccc(-c5nc(-c6ccccc6)nc(-c6ccc7oc8ccccc8c7c6)n5)c4)cc3)cc2)cc1 ; c1ccc(-c2ccc3c(-c4nc(-c5ccc6ccccc6c5)nc(-c5ccc6oc7ccccc7c6c5)n4)cccc3c2)cc1]

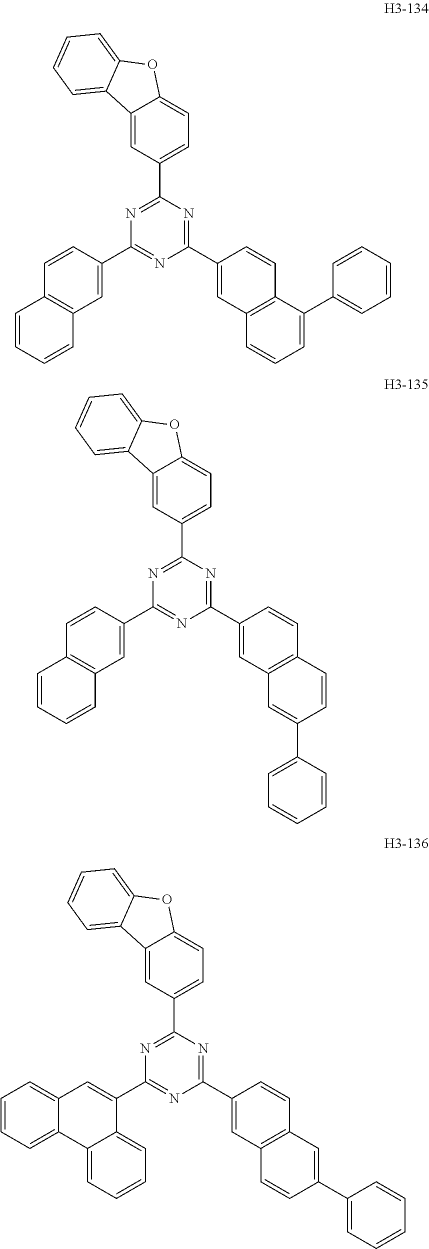 [SMILES: c1ccc(-c2cccc3cc(-c4nc(-c5ccc6ccccc6c5)nc(-c5ccc6oc7ccccc7c6c5)n4)ccc23)cc1 ; c1ccc(-c2ccc3ccc(-c4nc(-c5ccc6ccccc6c5)nc(-c5ccc6oc7ccccc7c6c5)n4)cc3c2)cc1 ; c1ccc(-c2ccc3cc(-c4nc(-c5ccc6oc7ccccc7c6c5)nc(-c5cc6ccccc6c6ccccc56)n4)ccc3c2)cc1]

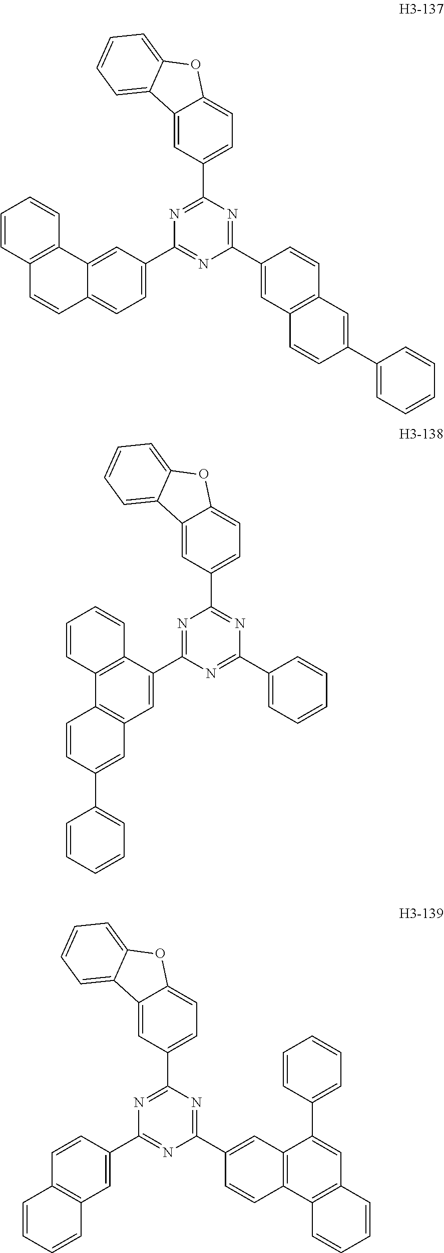 [SMILES: c1ccc(-c2ccc3cc(-c4nc(-c5ccc6ccc7ccccc7c6c5)nc(-c5ccc6oc7ccccc7c6c5)n4)ccc3c2)cc1 ; c1ccc(-c2ccc3c(c2)cc(-c2nc(-c4ccccc4)nc(-c4ccc5oc6ccccc6c5c4)n2)c2ccccc23)cc1 ; c1ccc(-c2cc3ccccc3c3ccc(-c4nc(-c5ccc6ccccc6c5)nc(-c5ccc6oc7ccccc7c6c5)n4)cc23)cc1]

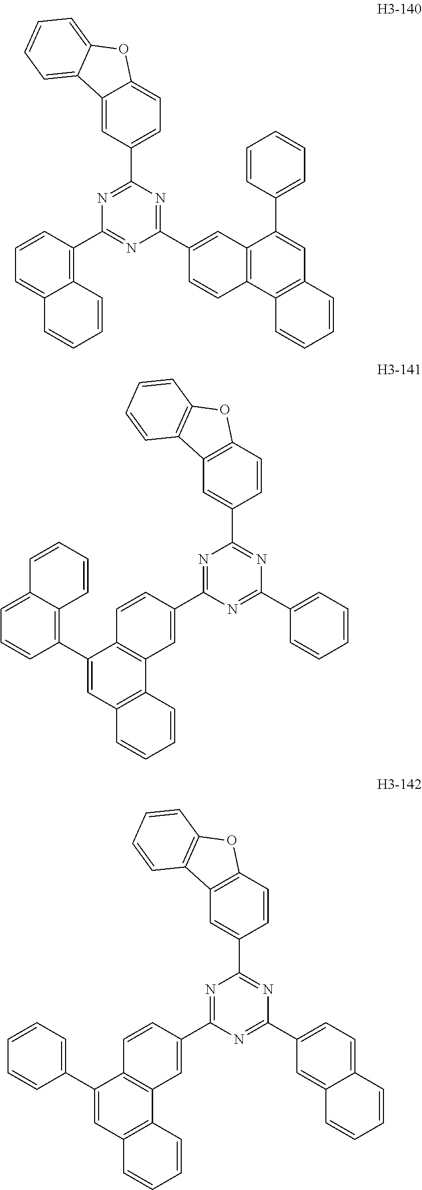 [SMILES: c1ccc(-c2cc3ccccc3c3ccc(-c4nc(-c5ccc6oc7ccccc7c6c5)nc(-c5cccc6ccccc56)n4)cc23)cc1 ; c1ccc(-c2nc(-c3ccc4oc5ccccc5c4c3)nc(-c3ccc4c(-c5cccc6ccccc56)cc5ccccc5c4c3)n2)cc1 ; c1ccc(-c2cc3ccccc3c3cc(-c4nc(-c5ccc6ccccc6c5)nc(-c5ccc6oc7ccccc7c6c5)n4)ccc23)cc1]

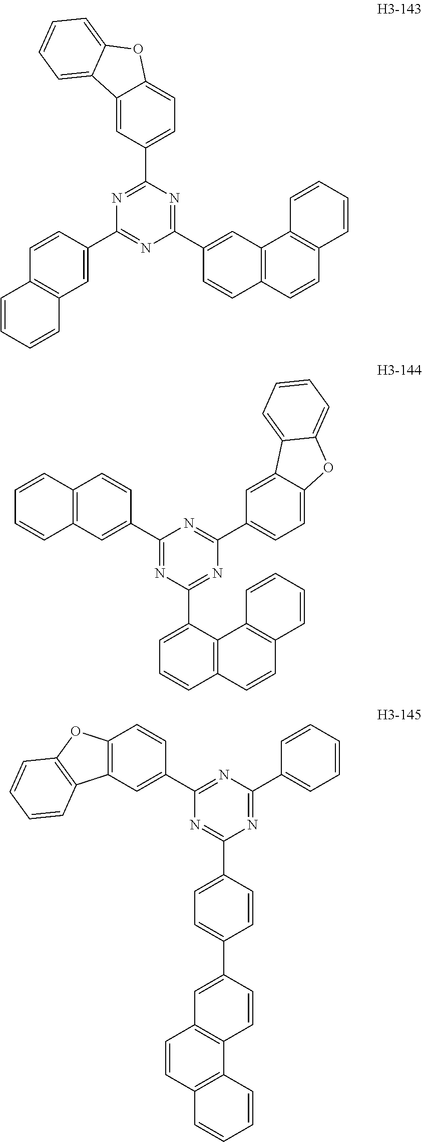 [SMILES: c1ccc2cc(-c3nc(-c4ccc5ccc6ccccc6c5c4)nc(-c4ccc5oc6ccccc6c5c4)n3)ccc2c1 ; c1ccc2cc(-c3nc(-c4ccc5oc6ccccc6c5c4)nc(-c4cccc5ccc6ccccc6c45)n3)ccc2c1 ; c1ccc(-c2nc(-c3ccc(-c4ccc5c(ccc6ccccc65)c4)cc3)nc(-c3ccc4oc5ccccc5c4c3)n2)cc1]

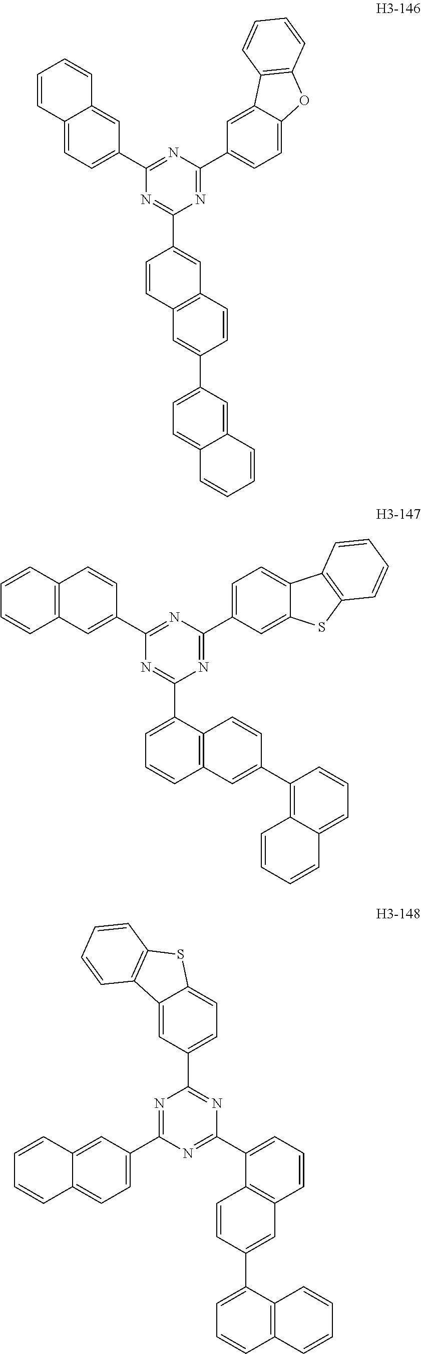 [SMILES: c1ccc2cc(-c3ccc4cc(-c5nc(-c6ccc7ccccc7c6)nc(-c6ccc7oc8ccccc8c7c6)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1 ; c1ccc2cc(-c3nc(-c4ccc5sc6ccccc6c5c4)nc(-c4cccc5cc(-c6cccc7ccccc67)ccc45)n3)ccc2c1]

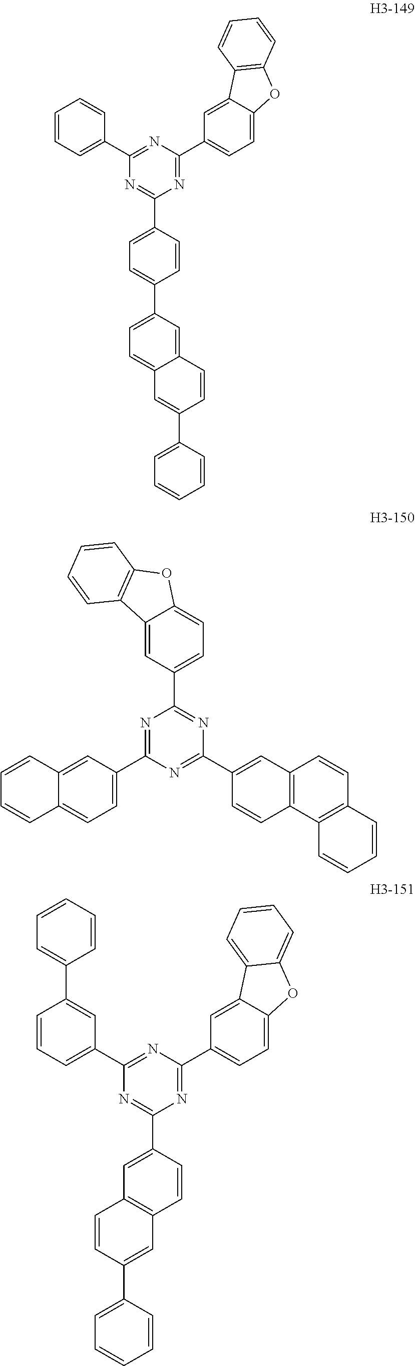 [SMILES: c1ccc(-c2ccc3cc(-c4ccc(-c5nc(-c6ccccc6)nc(-c6ccc7oc8ccccc8c7c6)n5)cc4)ccc3c2)cc1 ; c1ccc2cc(-c3nc(-c4ccc5c(ccc6ccccc65)c4)nc(-c4ccc5oc6ccccc6c5c4)n3)ccc2c1 ; c1ccc(-c2cccc(-c3nc(-c4ccc5cc(-c6ccccc6)ccc5c4)nc(-c4ccc5oc6ccccc6c5c4)n3)c2)cc1]

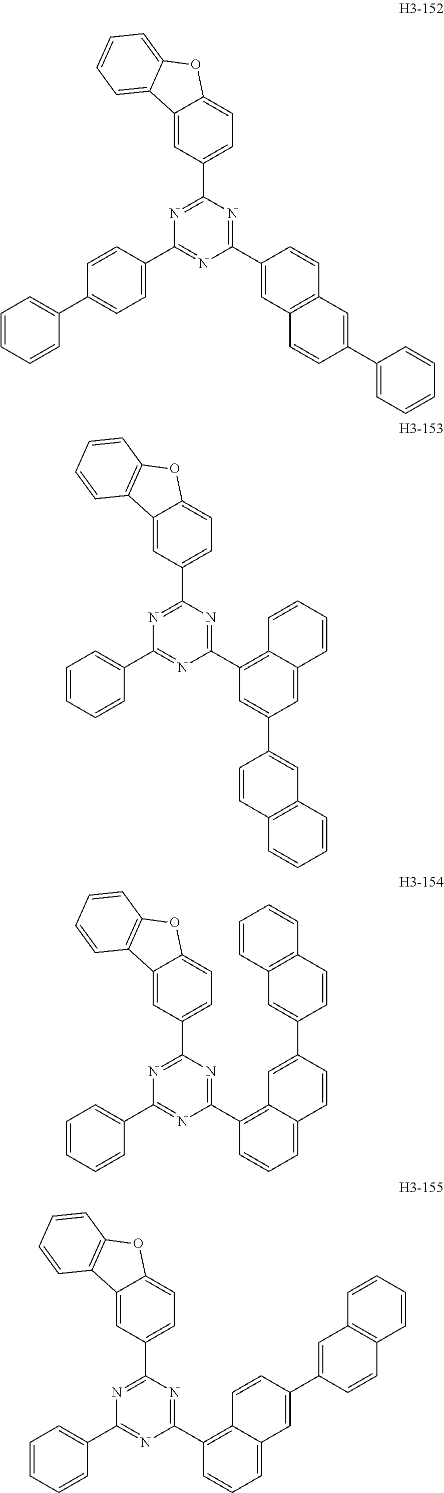

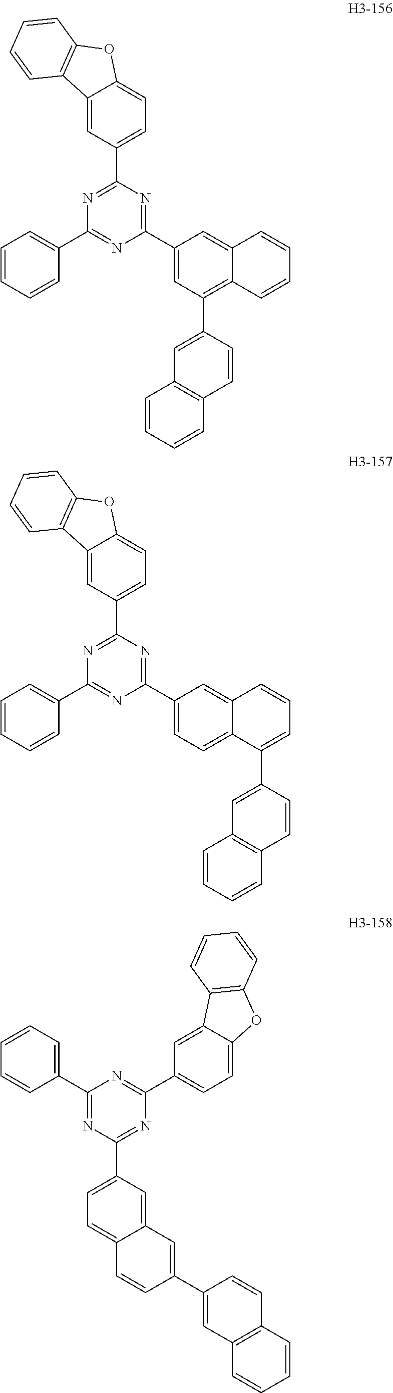

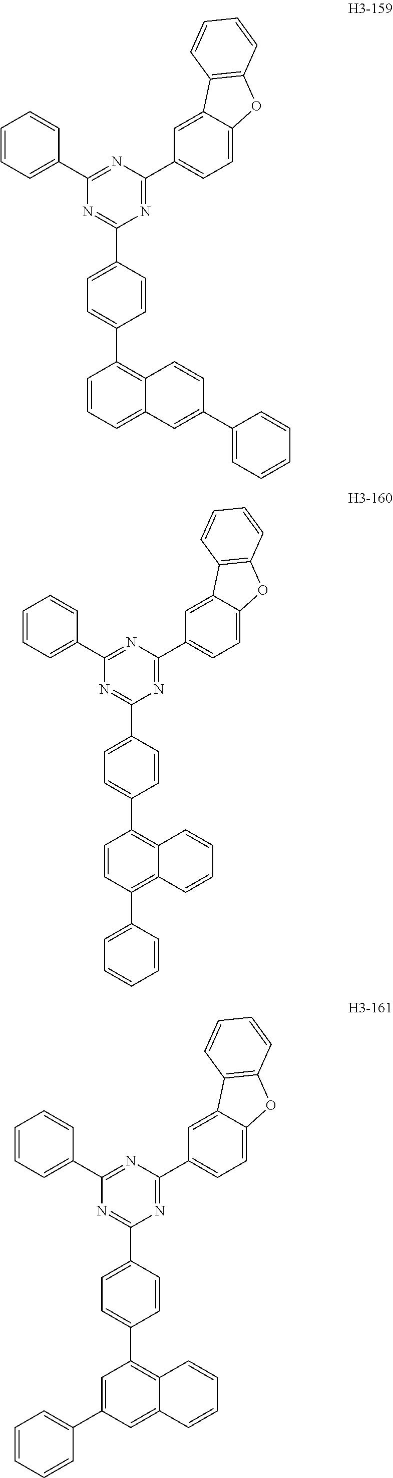

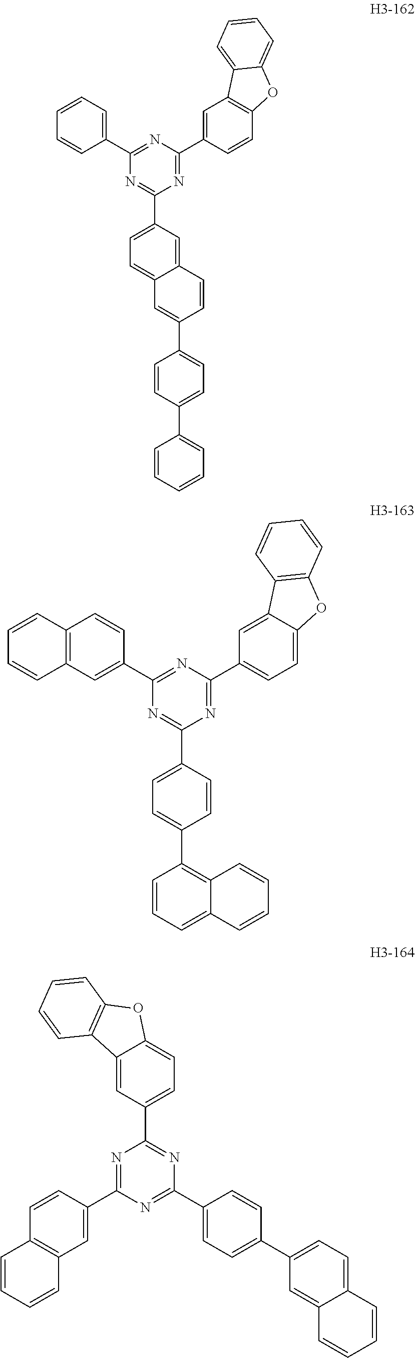 [SMILES: c1ccc(-c2ccc(-c3ccc4cc(-c5nc(-c6ccccc6)nc(-c6ccc7oc8ccccc8c7c6)n5)ccc4c3)cc2)cc1 ; c1ccc2cc(-c3nc(-c4ccc(-c5cccc6ccccc56)cc4)nc(-c4ccc5oc6ccccc6c5c4)n3)ccc2c1 ; c1ccc2cc(-c3ccc(-c4nc(-c5ccc6ccccc6c5)nc(-c5ccc6oc7ccccc7c6c5)n4)cc3)ccc2c1]

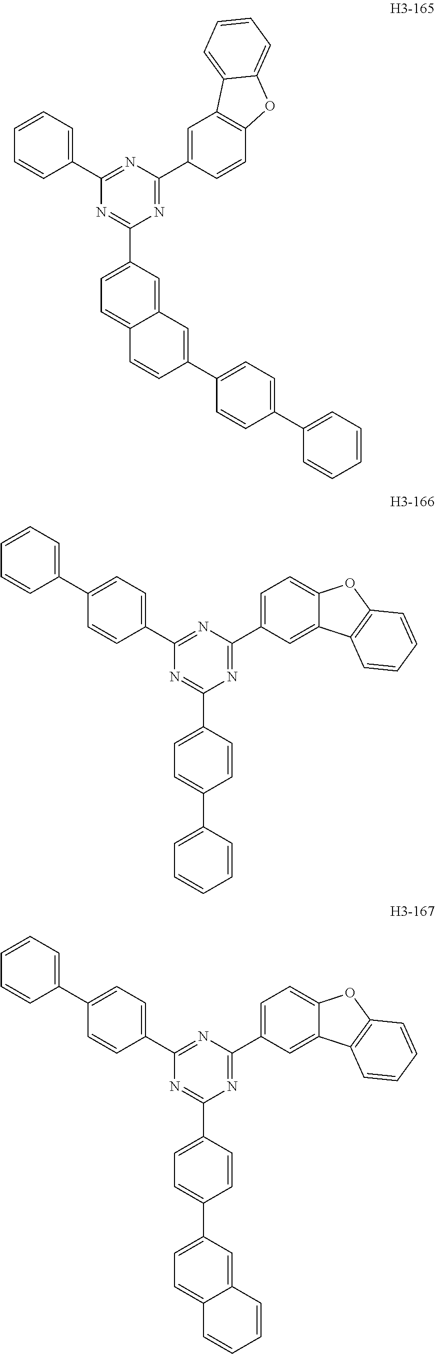 [SMILES: c1ccc(-c2ccc(-c3ccc4ccc(-c5nc(-c6ccccc6)nc(-c6ccc7oc8ccccc8c7c6)n5)cc4c3)cc2)cc1 ; c1ccc(-c2ccc(-c3nc(-c4ccc(-c5ccccc5)cc4)nc(-c4ccc5oc6ccccc6c5c4)n3)cc2)cc1 ; c1ccc(-c2ccc(-c3nc(-c4ccc(-c5ccc6ccccc6c5)cc4)nc(-c4ccc5oc6ccccc6c5c4)n3)cc2)cc1]

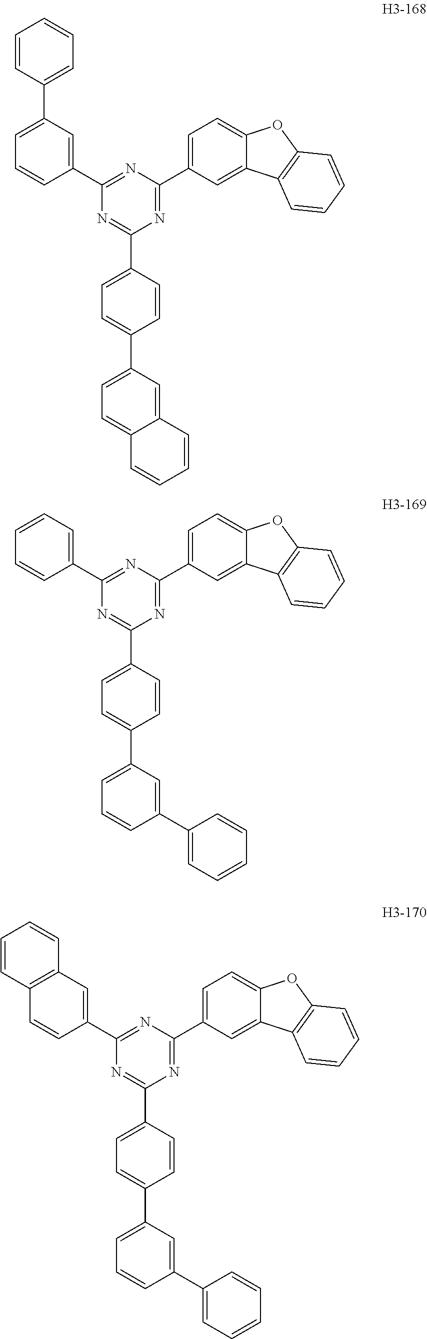 [SMILES: c1ccc(-c2cccc(-c3nc(-c4ccc(-c5ccc6ccccc6c5)cc4)nc(-c4ccc5oc6ccccc6c5c4)n3)c2)cc1 ; c1ccc(-c2cccc(-c3ccc(-c4nc(-c5ccccc5)nc(-c5ccc6oc7ccccc7c6c5)n4)cc3)c2)cc1 ; c1ccc(-c2cccc(-c3ccc(-c4nc(-c5ccc6ccccc6c5)nc(-c5ccc6oc7ccccc7c6c5)n4)cc3)c2)cc1]

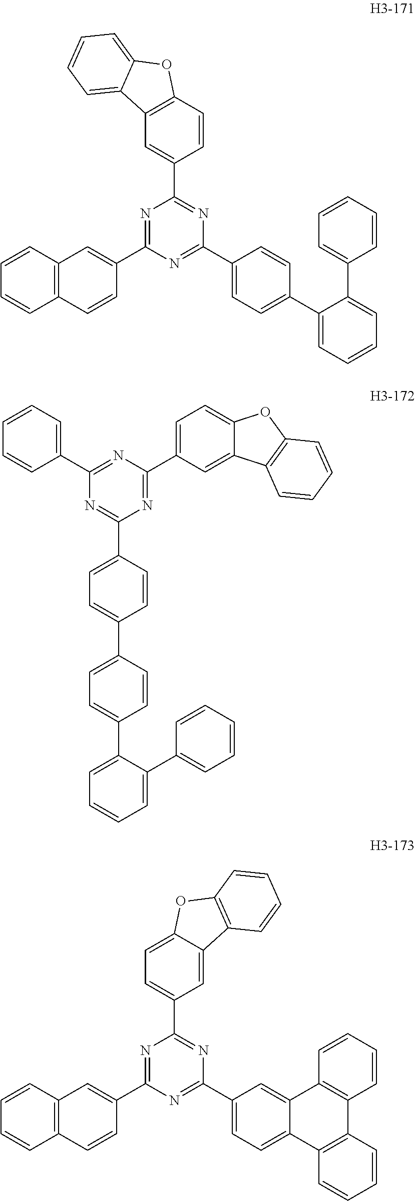 [SMILES: c1ccc(-c2ccccc2-c2ccc(-c3nc(-c4ccc5ccccc5c4)nc(-c4ccc5oc6ccccc6c5c4)n3)cc2)cc1 ; c1ccc(-c2nc(-c3ccc(-c4ccc(-c5ccccc5-c5ccccc5)cc4)cc3)nc(-c3ccc4oc5ccccc5c4c3)n2)cc1 ; c1ccc2cc(-c3nc(-c4ccc5oc6ccccc6c5c4)nc(-c4ccc5c6ccccc6c6ccccc6c5c4)n3)ccc2c1]

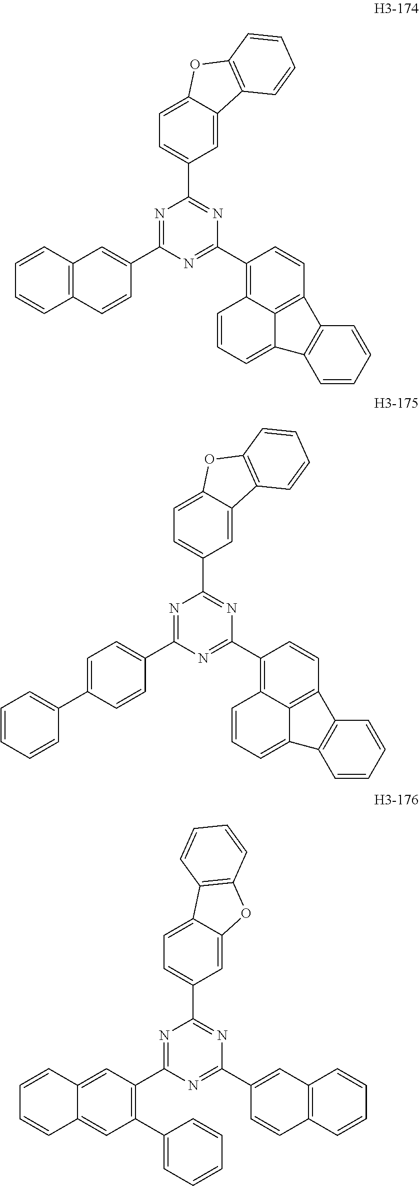 [SMILES: c1ccc2c(c1)-c1cccc3c(-c4nc(-c5ccc6ccccc6c5)nc(-c5ccc6oc7ccccc7c6c5)n4)ccc-2c13 ; c1ccc(-c2ccc(-c3nc(-c4ccc5oc6ccccc6c5c4)nc(-c4ccc5c6c(cccc46)-c4ccccc4-5)n3)cc2)cc1 ; c1ccc(-c2cc3ccccc3cc2-c2nc(-c3ccc4ccccc4c3)nc(-c3ccc4c(c3)oc3ccccc34)n2)cc1]

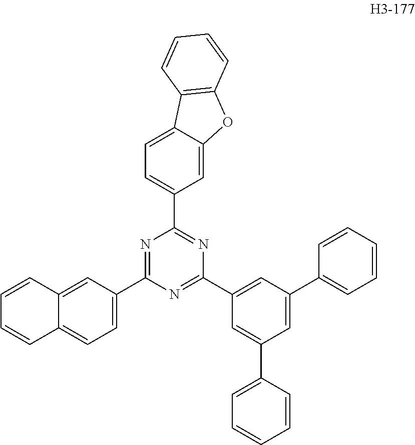 [SMILES: c1ccc(-c2cc(-c3ccccc3)cc(-c3nc(-c4ccc5ccccc5c4)nc(-c4ccc5c(c4)oc4ccccc45)n3)c2)cc1]

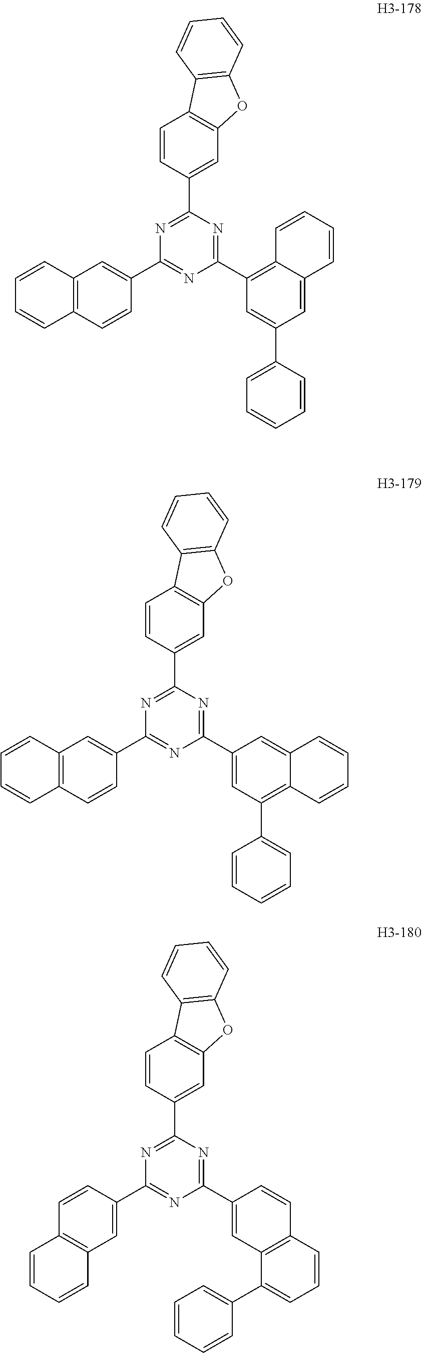 [SMILES: c1ccc(-c2cc(-c3nc(-c4ccc5ccccc5c4)nc(-c4ccc5c(c4)oc4ccccc45)n3)c3ccccc3c2)cc1 ; c1ccc(-c2cc(-c3nc(-c4ccc5ccccc5c4)nc(-c4ccc5c(c4)oc4ccccc45)n3)cc3ccccc23)cc1 ; c1ccc(-c2cccc3ccc(-c4nc(-c5ccc6ccccc6c5)nc(-c5ccc6c(c5)oc5ccccc56)n4)cc23)cc1]

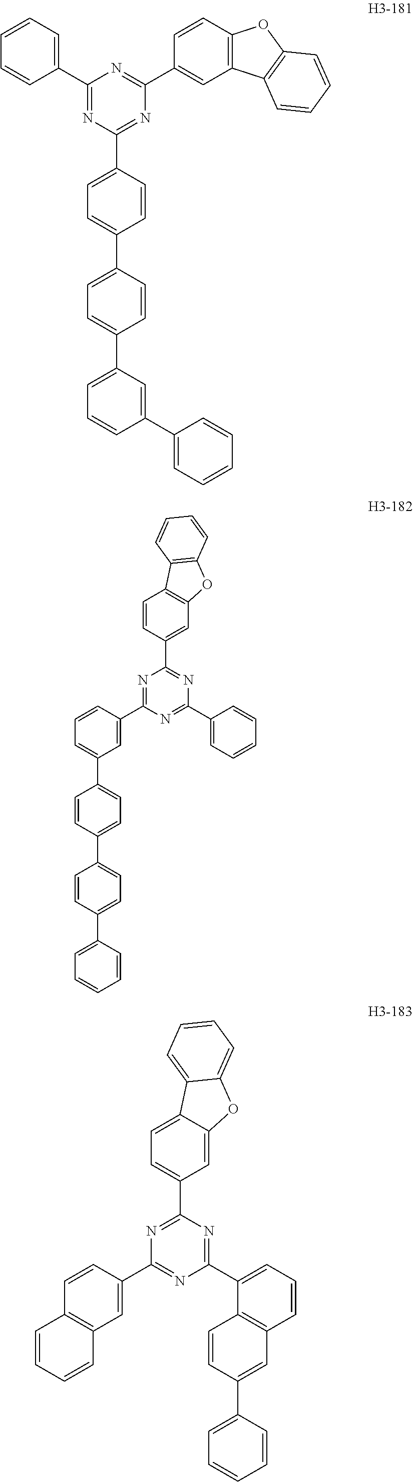

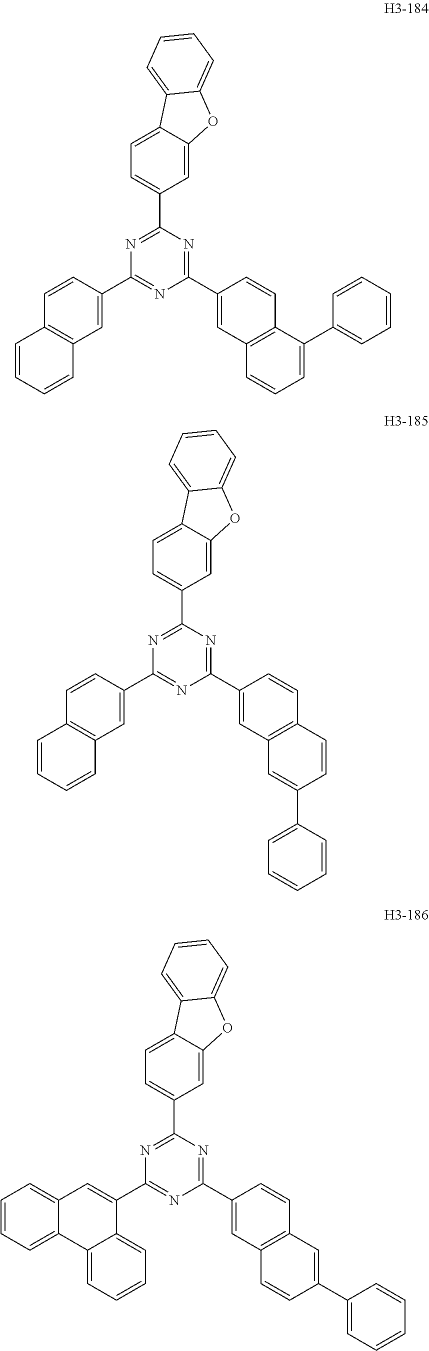 [SMILES: c1ccc(-c2cccc3cc(-c4nc(-c5ccc6ccccc6c5)nc(-c5ccc6c(c5)oc5ccccc56)n4)ccc23)cc1 ; c1ccc(-c2ccc3ccc(-c4nc(-c5ccc6ccccc6c5)nc(-c5ccc6c(c5)oc5ccccc56)n4)cc3c2)cc1 ; c1ccc(-c2ccc3cc(-c4nc(-c5ccc6c(c5)oc5ccccc56)nc(-c5cc6ccccc6c6ccccc56)n4)ccc3c2)cc1]

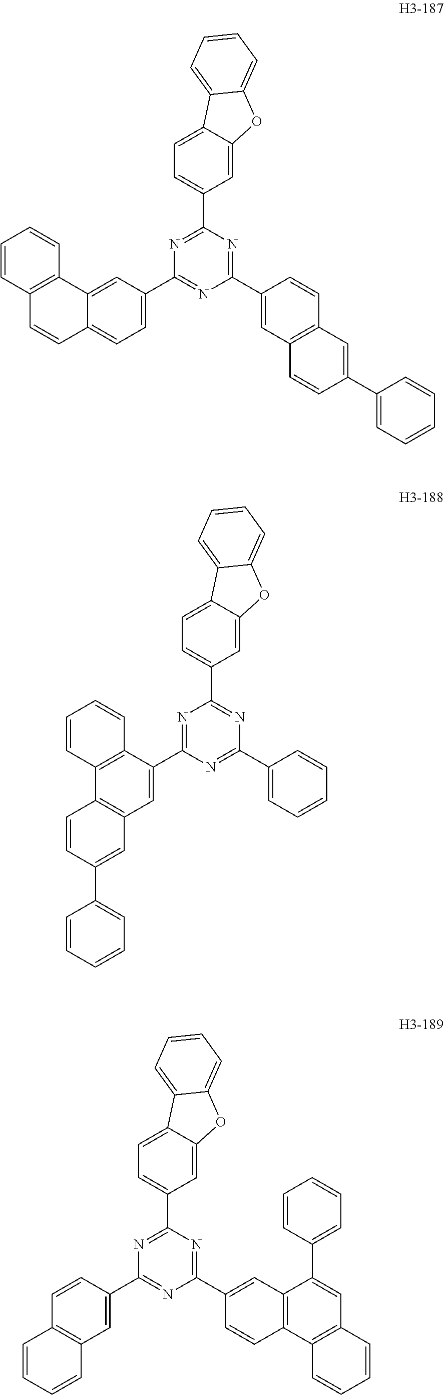 [SMILES: c1ccc(-c2ccc3cc(-c4nc(-c5ccc6c(c5)oc5ccccc56)nc(-c5ccc6ccc7ccccc7c6c5)n4)ccc3c2)cc1 ; c1ccc(-c2ccc3c(c2)cc(-c2nc(-c4ccccc4)nc(-c4ccc5c(c4)oc4ccccc45)n2)c2ccccc23)cc1 ; c1ccc(-c2cc3ccccc3c3ccc(-c4nc(-c5ccc6ccccc6c5)nc(-c5ccc6c(c5)oc5ccccc56)n4)cc23)cc1]

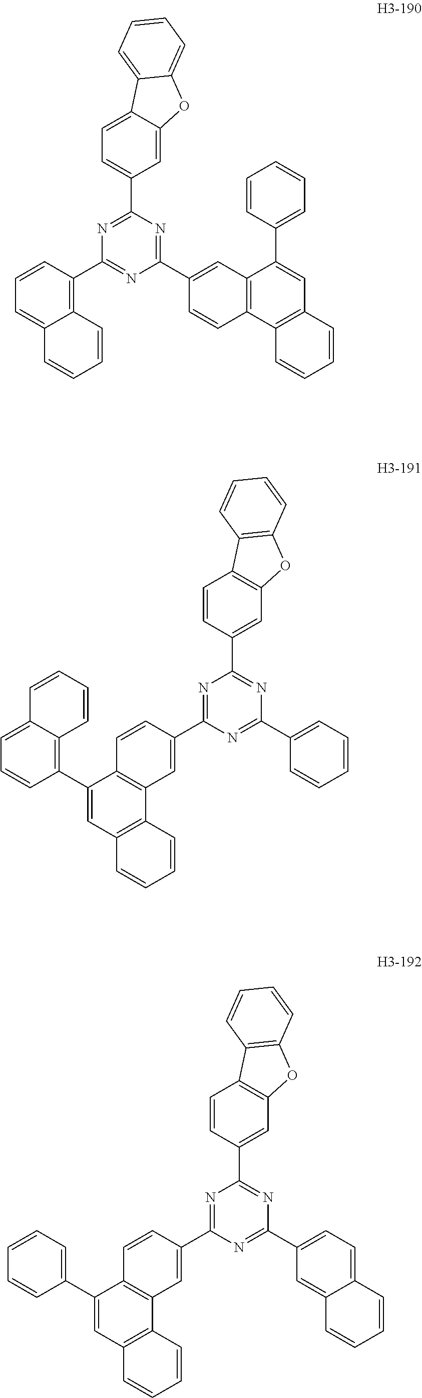 [SMILES: c1ccc(-c2cc3ccccc3c3ccc(-c4nc(-c5ccc6c(c5)oc5ccccc56)nc(-c5cccc6ccccc56)n4)cc23)cc1 ; c1ccc(-c2nc(-c3ccc4c(c3)oc3ccccc34)nc(-c3ccc4c(-c5cccc6ccccc56)cc5ccccc5c4c3)n2)cc1 ; c1ccc(-c2cc3ccccc3c3cc(-c4nc(-c5ccc6ccccc6c5)nc(-c5ccc6c(c5)oc5ccccc56)n4)ccc23)cc1]

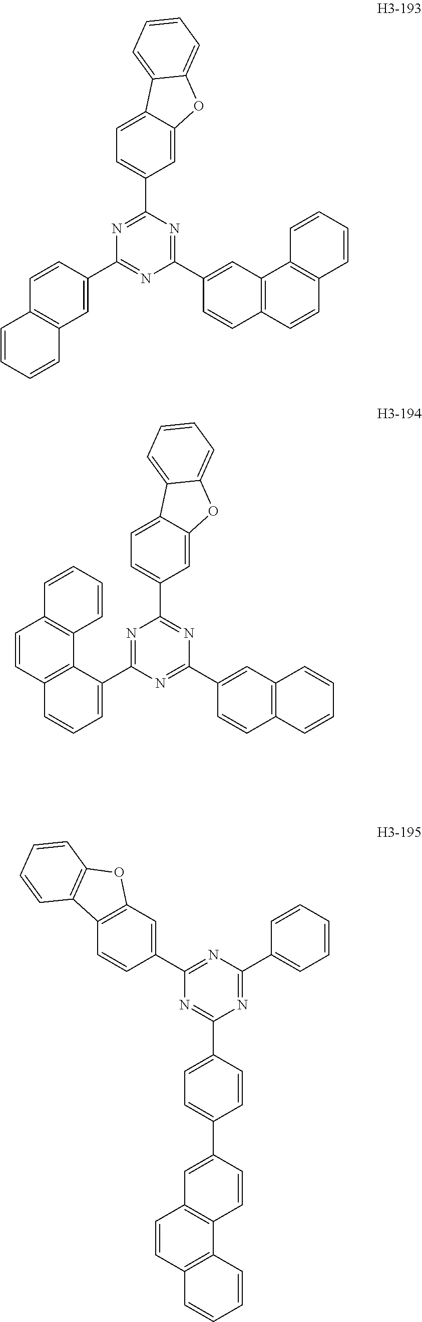 [SMILES: c1ccc2cc(-c3nc(-c4ccc5c(c4)oc4ccccc45)nc(-c4ccc5ccc6ccccc6c5c4)n3)ccc2c1 ; c1ccc2cc(-c3nc(-c4ccc5c(c4)oc4ccccc45)nc(-c4cccc5ccc6ccccc6c45)n3)ccc2c1 ; c1ccc(-c2nc(-c3ccc(-c4ccc5c(ccc6ccccc65)c4)cc3)nc(-c3ccc4c(c3)oc3ccccc34)n2)cc1]

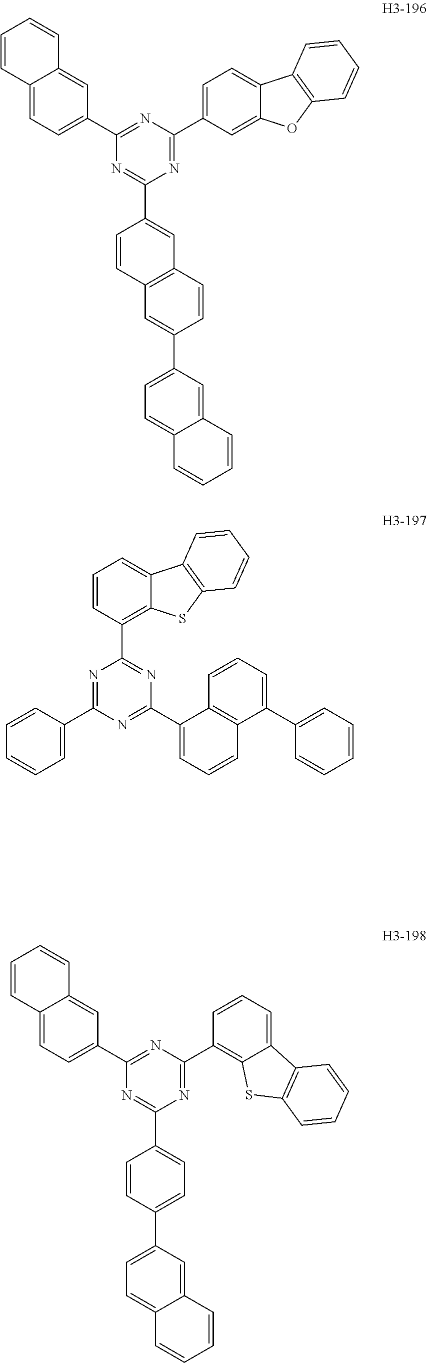 [SMILES: c1ccc2cc(-c3ccc4cc(-c5nc(-c6ccc7ccccc7c6)nc(-c6ccc7c(c6)oc6ccccc67)n5)ccc4c3)ccc2c1 ; c1ccc(-c2nc(-c3cccc4c(-c5ccccc5)cccc34)nc(-c3cccc4c3sc3ccccc34)n2)cc1 ; c1ccc2cc(-c3ccc(-c4nc(-c5ccc6ccccc6c5)nc(-c5cccc6c5sc5ccccc56)n4)cc3)ccc2c1]

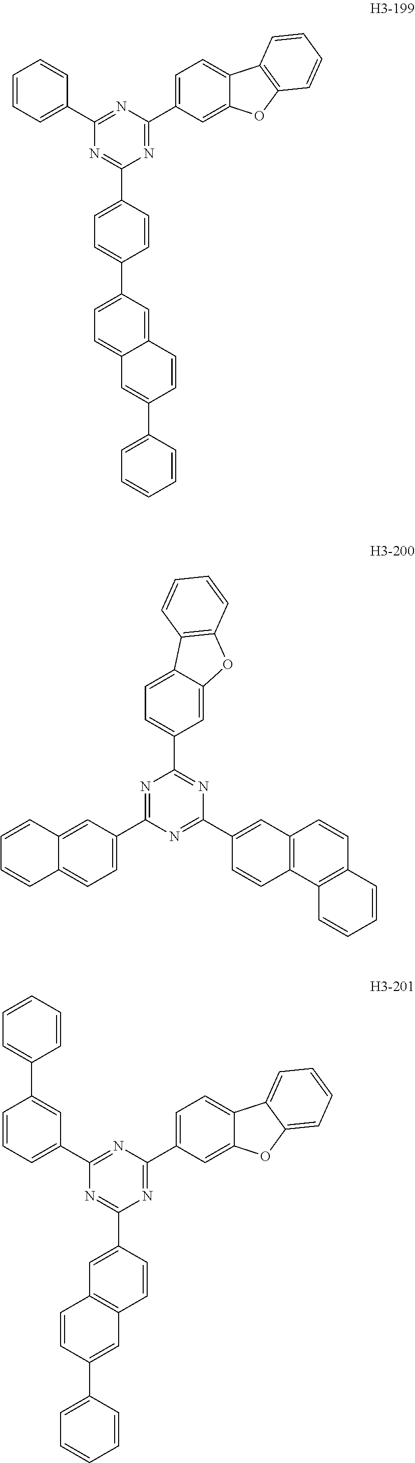 [SMILES: c1ccc(-c2ccc3cc(-c4ccc(-c5nc(-c6ccccc6)nc(-c6ccc7c(c6)oc6ccccc67)n5)cc4)ccc3c2)cc1 ; c1ccc2cc(-c3nc(-c4ccc5c(ccc6ccccc65)c4)nc(-c4ccc5c(c4)oc4ccccc45)n3)ccc2c1 ; c1ccc(-c2cccc(-c3nc(-c4ccc5cc(-c6ccccc6)ccc5c4)nc(-c4ccc5c(c4)oc4ccccc45)n3)c2)cc1]

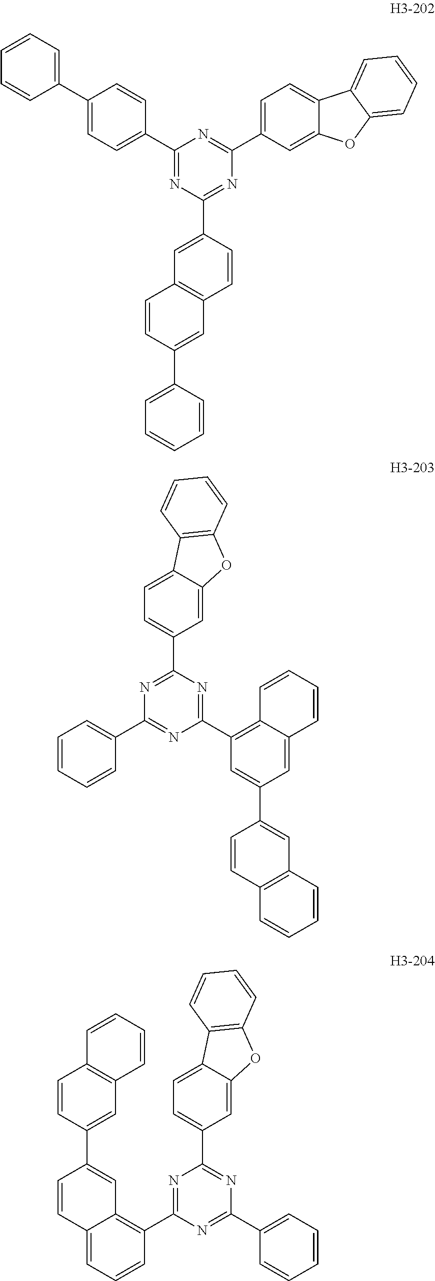

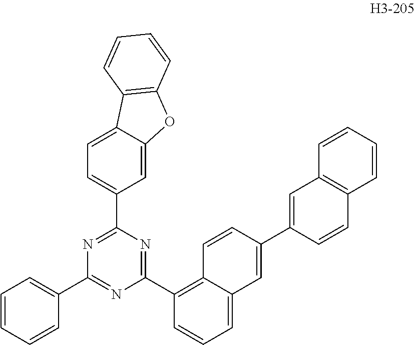 [SMILES: c1ccc(-c2nc(-c3ccc4c(c3)oc3ccccc34)nc(-c3cccc4cc(-c5ccc6ccccc6c5)ccc34)n2)cc1]

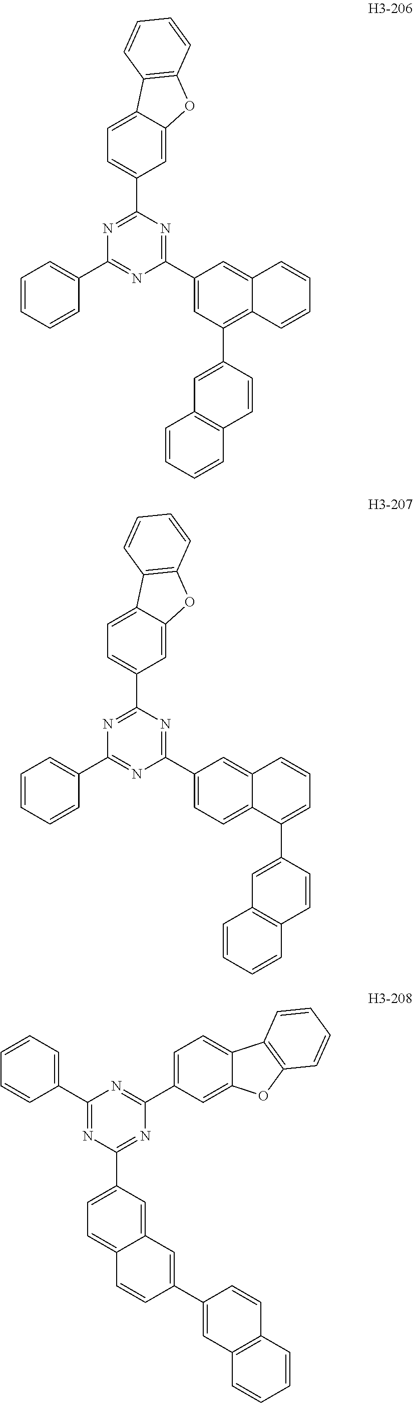

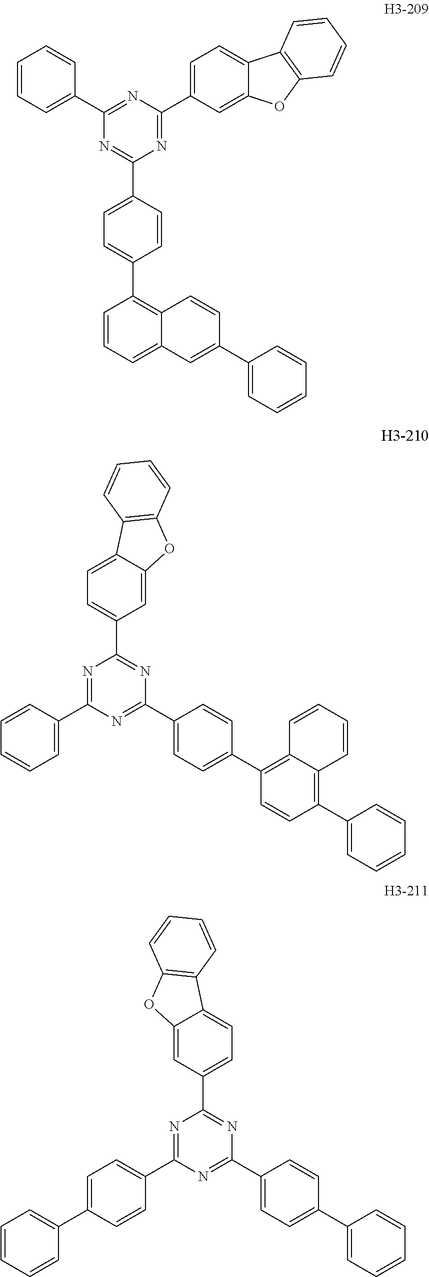 [SMILES: c1ccc(-c2ccc3c(-c4ccc(-c5nc(-c6ccccc6)nc(-c6ccc7c(c6)oc6ccccc67)n5)cc4)cccc3c2)cc1 ; c1ccc(-c2nc(-c3ccc(-c4ccc(-c5ccccc5)c5ccccc45)cc3)nc(-c3ccc4c(c3)oc3ccccc34)n2)cc1 ; c1ccc(-c2ccc(-c3nc(-c4ccc(-c5ccccc5)cc4)nc(-c4ccc5c(c4)oc4ccccc45)n3)cc2)cc1]

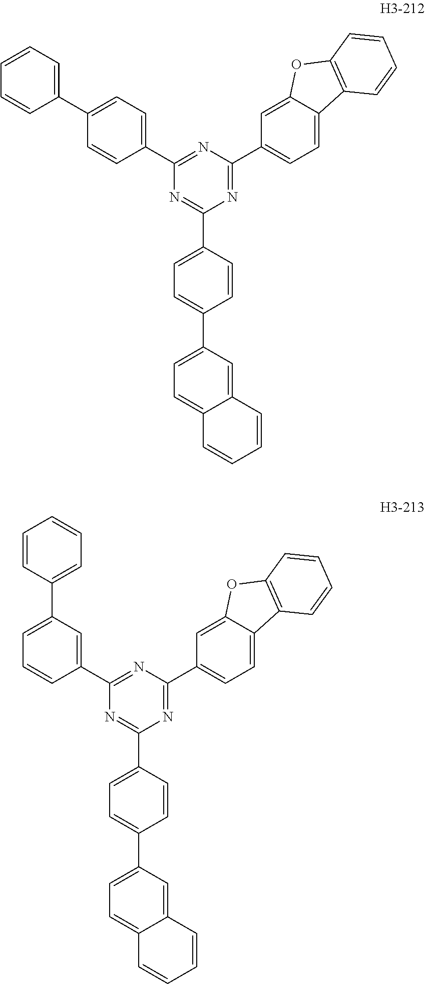 [SMILES: c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2cccc(-c3nc(-c4ccc(-c5ccc6ccccc6c5)cc4)nc(-c4ccc5c(c4)oc4ccccc45)n3)c2)cc1]

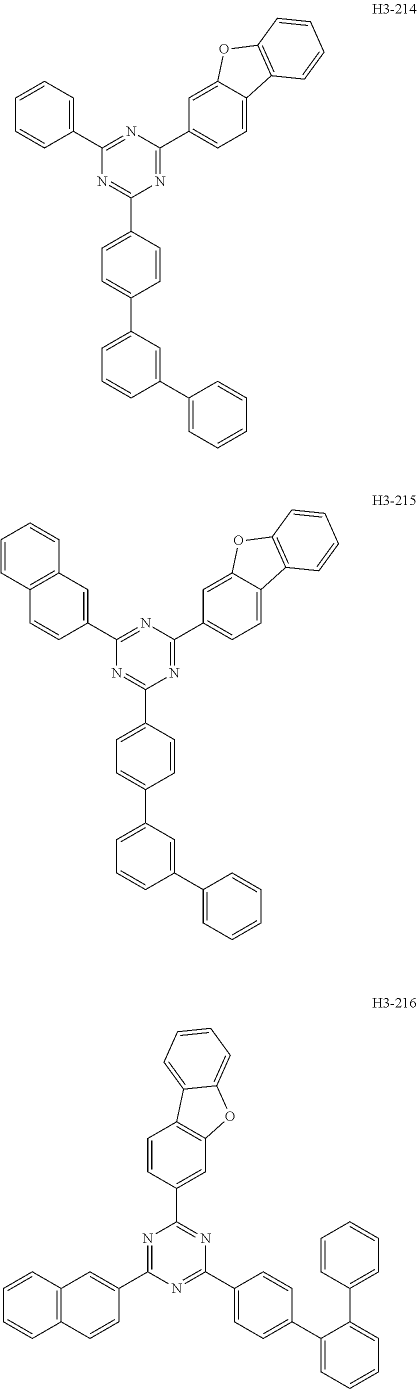 [SMILES: c1ccc(-c2cccc(-c3ccc(-c4nc(-c5ccccc5)nc(-c5ccc6c(c5)oc5ccccc56)n4)cc3)c2)cc1 ; c1ccc(-c2cccc(-c3ccc(-c4nc(-c5ccc6ccccc6c5)nc(-c5ccc6c(c5)oc5ccccc56)n4)cc3)c2)cc1 ; c1ccc(-c2ccccc2-c2ccc(-c3nc(-c4ccc5ccccc5c4)nc(-c4ccc5c(c4)oc4ccccc45)n3)cc2)cc1]

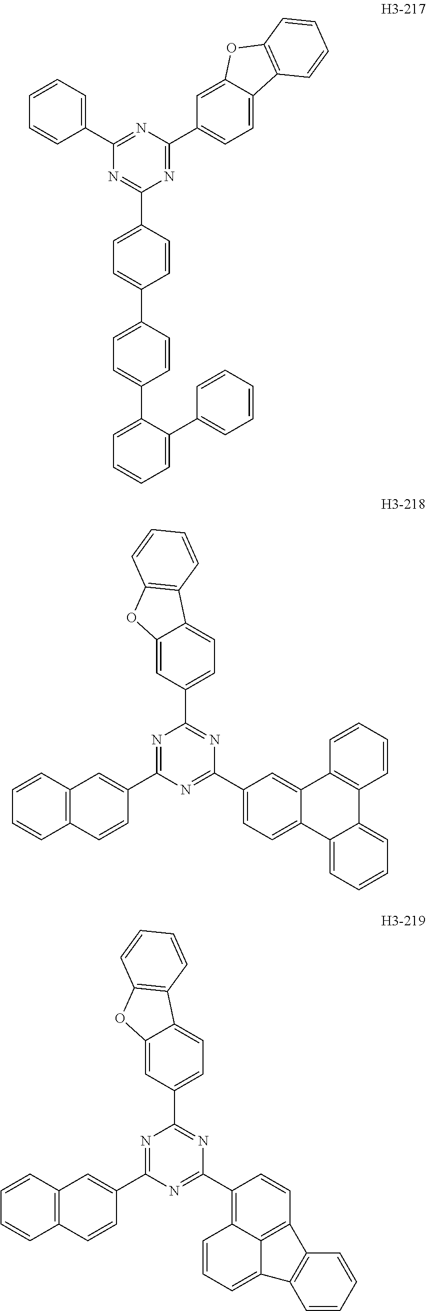 [SMILES: c1ccc(-c2nc(-c3ccc(-c4ccc(-c5ccccc5-c5ccccc5)cc4)cc3)nc(-c3ccc4c(c3)oc3ccccc34)n2)cc1 ; c1ccc2cc(-c3nc(-c4ccc5c(c4)oc4ccccc45)nc(-c4ccc5c6ccccc6c6ccccc6c5c4)n3)ccc2c1 ; c1ccc2c(c1)-c1cccc3c(-c4nc(-c5ccc6ccccc6c5)nc(-c5ccc6c(c5)oc5ccccc56)n4)ccc-2c13]

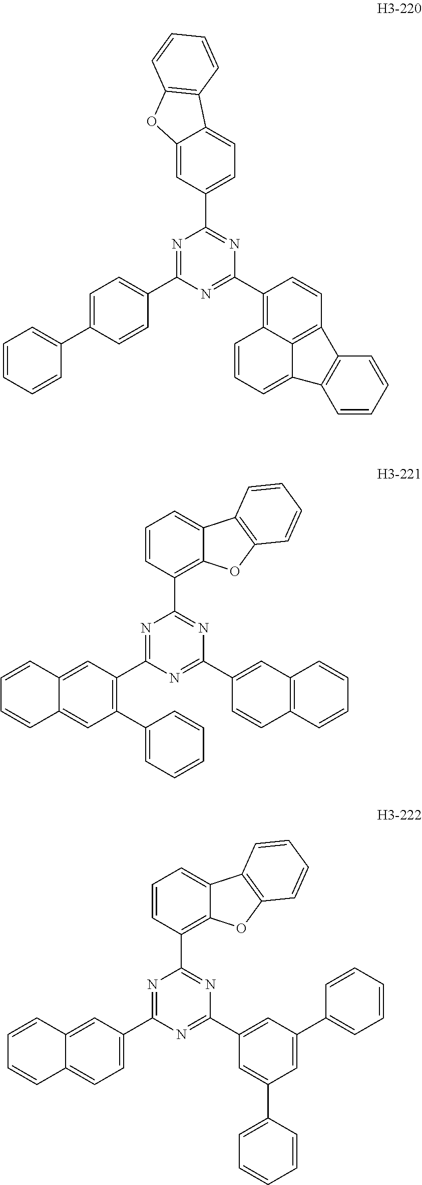 [SMILES: c1ccc(-c2ccc(-c3nc(-c4ccc5c(c4)oc4ccccc45)nc(-c4ccc5c6c(cccc46)-c4ccccc4-5)n3)cc2)cc1 ; c1ccc(-c2cc3ccccc3cc2-c2nc(-c3ccc4ccccc4c3)nc(-c3cccc4c3oc3ccccc34)n2)cc1 ; c1ccc(-c2cc(-c3ccccc3)cc(-c3nc(-c4ccc5ccccc5c4)nc(-c4cccc5c4oc4ccccc45)n3)c2)cc1]

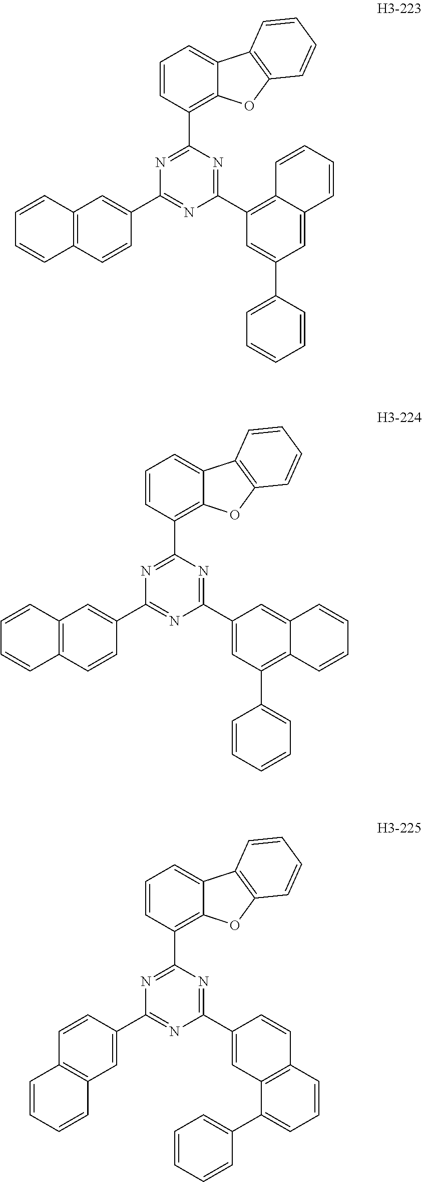 [SMILES: c1ccc(-c2cc(-c3nc(-c4ccc5ccccc5c4)nc(-c4cccc5c4oc4ccccc45)n3)c3ccccc3c2)cc1 ; c1ccc(-c2cc(-c3nc(-c4ccc5ccccc5c4)nc(-c4cccc5c4oc4ccccc45)n3)cc3ccccc23)cc1 ; c1ccc(-c2cccc3ccc(-c4nc(-c5ccc6ccccc6c5)nc(-c5cccc6c5oc5ccccc56)n4)cc23)cc1]

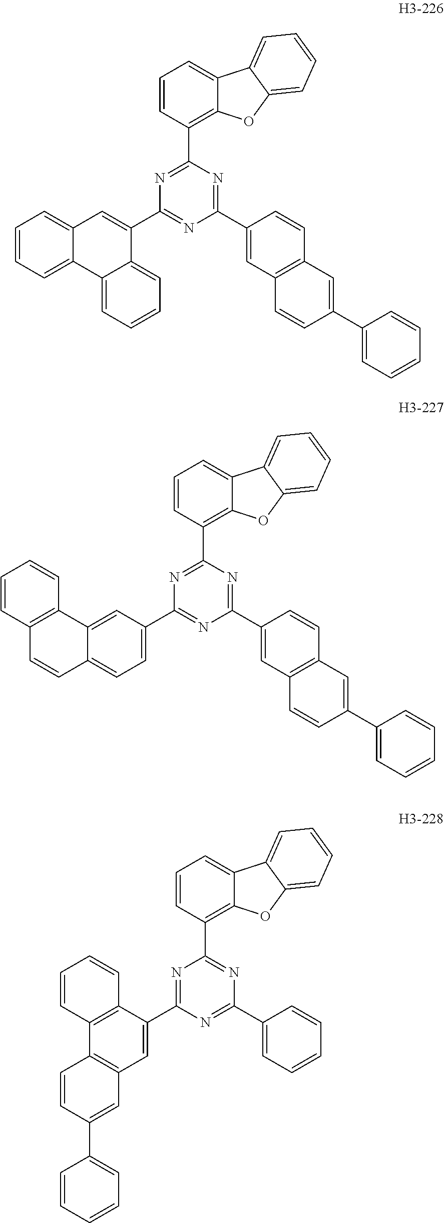 [SMILES: c1ccc(-c2ccc3cc(-c4nc(-c5cc6ccccc6c6ccccc56)nc(-c5cccc6c5oc5ccccc56)n4)ccc3c2)cc1 ; c1ccc(-c2ccc3cc(-c4nc(-c5ccc6ccc7ccccc7c6c5)nc(-c5cccc6c5oc5ccccc56)n4)ccc3c2)cc1 ; c1ccc(-c2ccc3c(c2)cc(-c2nc(-c4ccccc4)nc(-c4cccc5c4oc4ccccc45)n2)c2ccccc23)cc1]

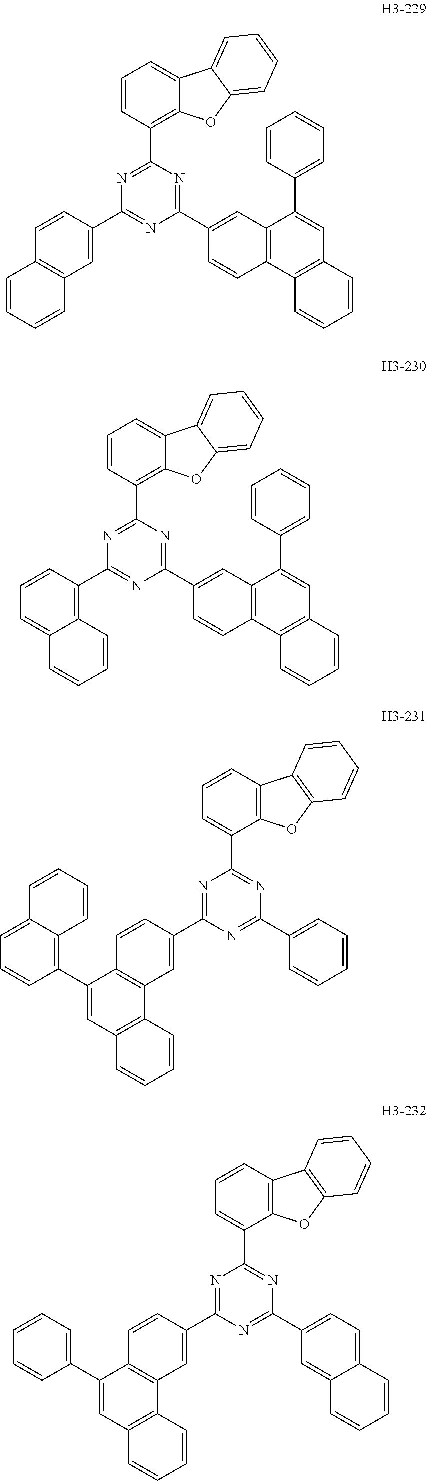 [SMILES: c1ccc(-c2cc3ccccc3c3ccc(-c4nc(-c5ccc6ccccc6c5)nc(-c5cccc6c5oc5ccccc56)n4)cc23)cc1 ; c1ccc(-c2cc3ccccc3c3ccc(-c4nc(-c5cccc6ccccc56)nc(-c5cccc6c5oc5ccccc56)n4)cc23)cc1 ; c1ccc(-c2nc(-c3ccc4c(-c5cccc6ccccc56)cc5ccccc5c4c3)nc(-c3cccc4c3oc3ccccc34)n2)cc1 ; c1ccc(-c2cc3ccccc3c3cc(-c4nc(-c5ccc6ccccc6c5)nc(-c5cccc6c5oc5ccccc56)n4)ccc23)cc1]

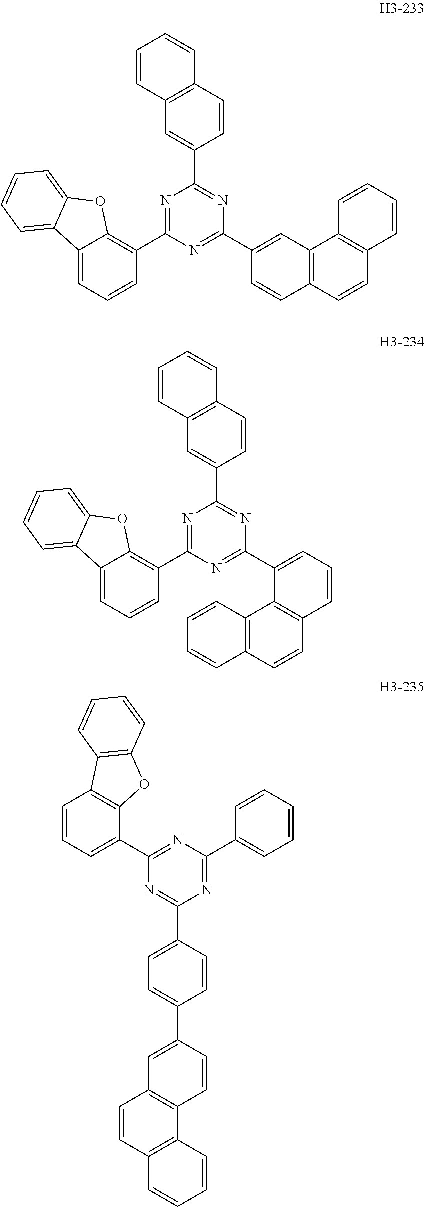 [SMILES: c1ccc2cc(-c3nc(-c4ccc5ccc6ccccc6c5c4)nc(-c4cccc5c4oc4ccccc45)n3)ccc2c1 ; c1ccc2cc(-c3nc(-c4cccc5c4oc4ccccc45)nc(-c4cccc5ccc6ccccc6c45)n3)ccc2c1 ; c1ccc(-c2nc(-c3ccc(-c4ccc5c(ccc6ccccc65)c4)cc3)nc(-c3cccc4c3oc3ccccc34)n2)cc1]

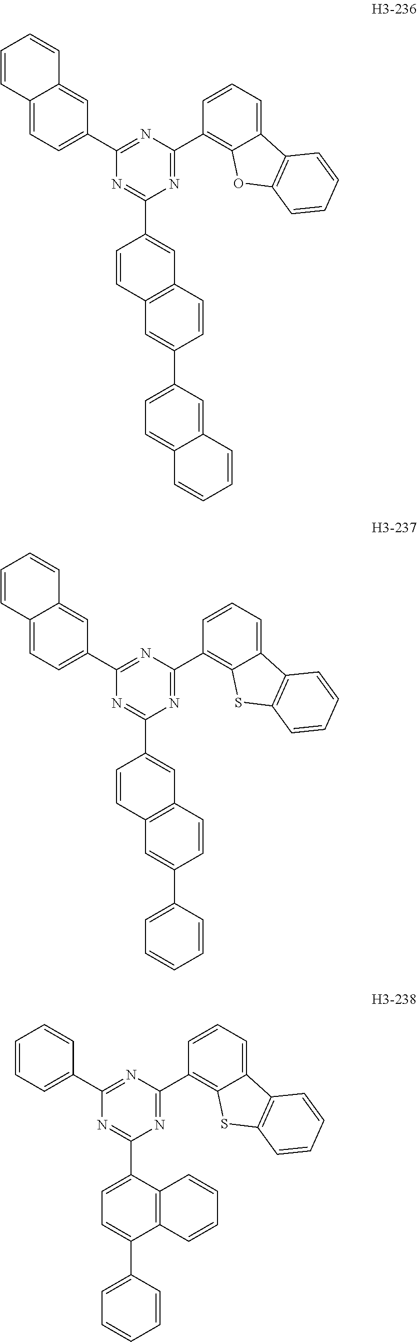 [SMILES: c1ccc2cc(-c3ccc4cc(-c5nc(-c6ccc7ccccc7c6)nc(-c6cccc7c6oc6ccccc67)n5)ccc4c3)ccc2c1 ; c1ccc(-c2ccc3cc(-c4nc(-c5ccc6ccccc6c5)nc(-c5cccc6c5sc5ccccc56)n4)ccc3c2)cc1 ; c1ccc(-c2nc(-c3ccc(-c4ccccc4)c4ccccc34)nc(-c3cccc4c3sc3ccccc34)n2)cc1]

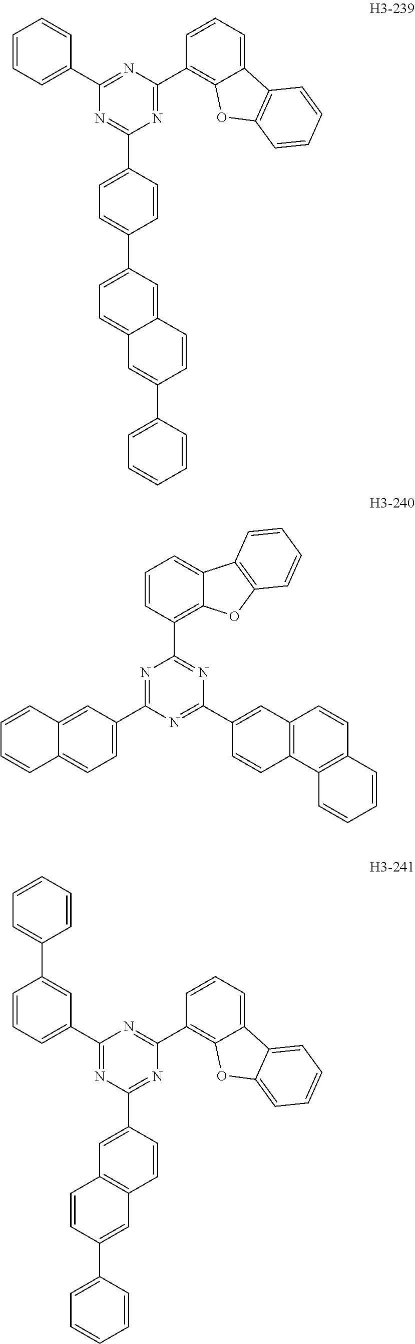 [SMILES: c1ccc(-c2ccc3cc(-c4ccc(-c5nc(-c6ccccc6)nc(-c6cccc7c6oc6ccccc67)n5)cc4)ccc3c2)cc1 ; c1ccc2cc(-c3nc(-c4ccc5c(ccc6ccccc65)c4)nc(-c4cccc5c4oc4ccccc45)n3)ccc2c1 ; c1ccc(-c2cccc(-c3nc(-c4ccc5cc(-c6ccccc6)ccc5c4)nc(-c4cccc5c4oc4ccccc45)n3)c2)cc1]

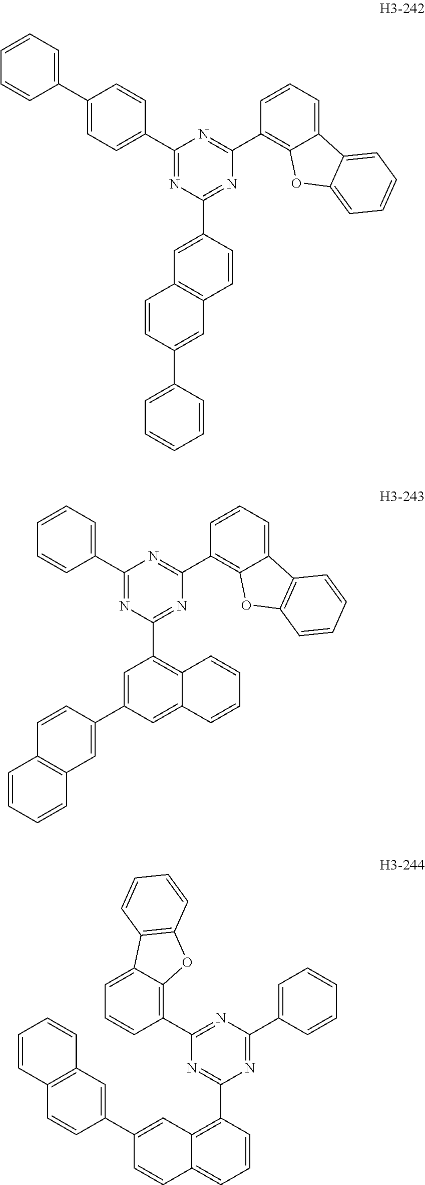 [SMILES: c1ccc(-c2ccc(-c3nc(-c4ccc5cc(-c6ccccc6)ccc5c4)nc(-c4cccc5c4oc4ccccc45)n3)cc2)cc1 ; c1ccc(-c2nc(-c3cc(-c4ccc5ccccc5c4)cc4ccccc34)nc(-c3cccc4c3oc3ccccc34)n2)cc1 ; c1ccc(-c2nc(-c3cccc4ccc(-c5ccc6ccccc6c5)cc34)nc(-c3cccc4c3oc3ccccc34)n2)cc1]

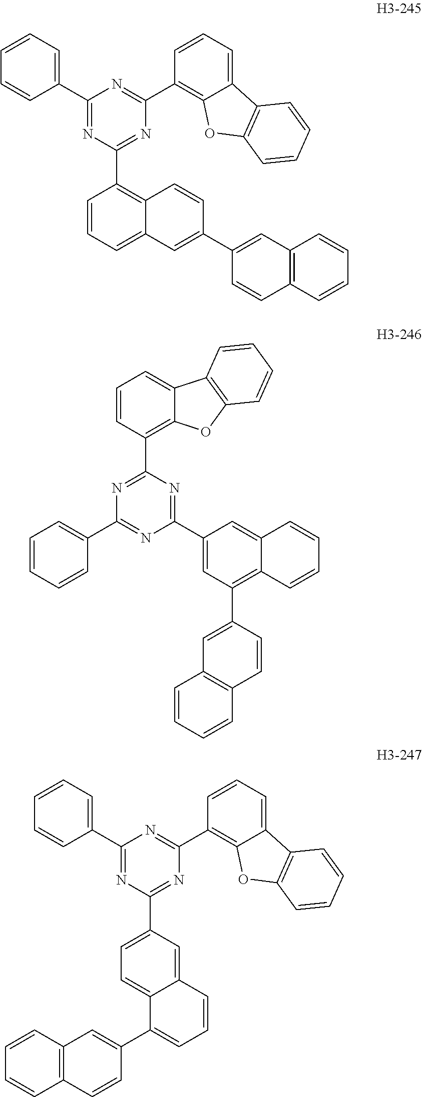

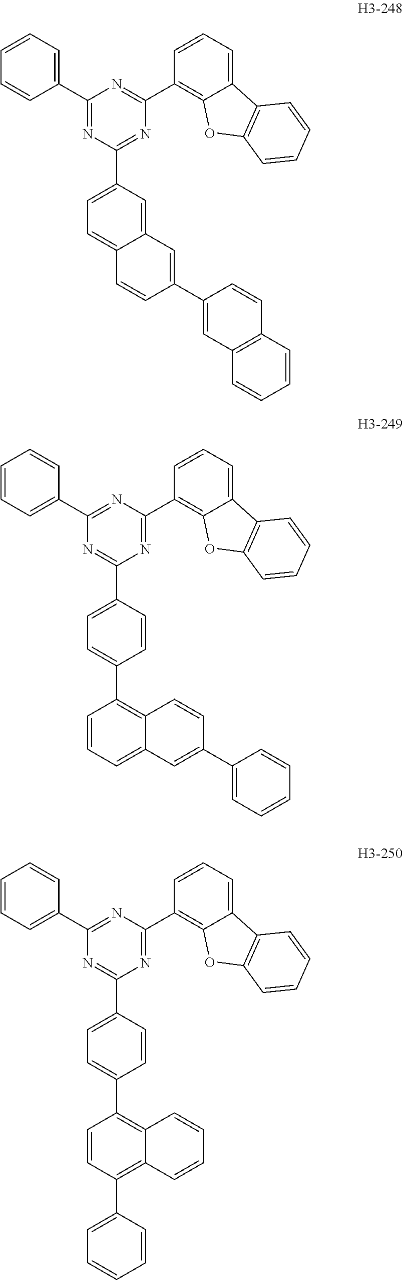 [SMILES: c1ccc(-c2nc(-c3ccc4ccc(-c5ccc6ccccc6c5)cc4c3)nc(-c3cccc4c3oc3ccccc34)n2)cc1 ; c1ccc(-c2ccc3c(-c4ccc(-c5nc(-c6ccccc6)nc(-c6cccc7c6oc6ccccc67)n5)cc4)cccc3c2)cc1 ; c1ccc(-c2nc(-c3ccc(-c4ccc(-c5ccccc5)c5ccccc45)cc3)nc(-c3cccc4c3oc3ccccc34)n2)cc1]

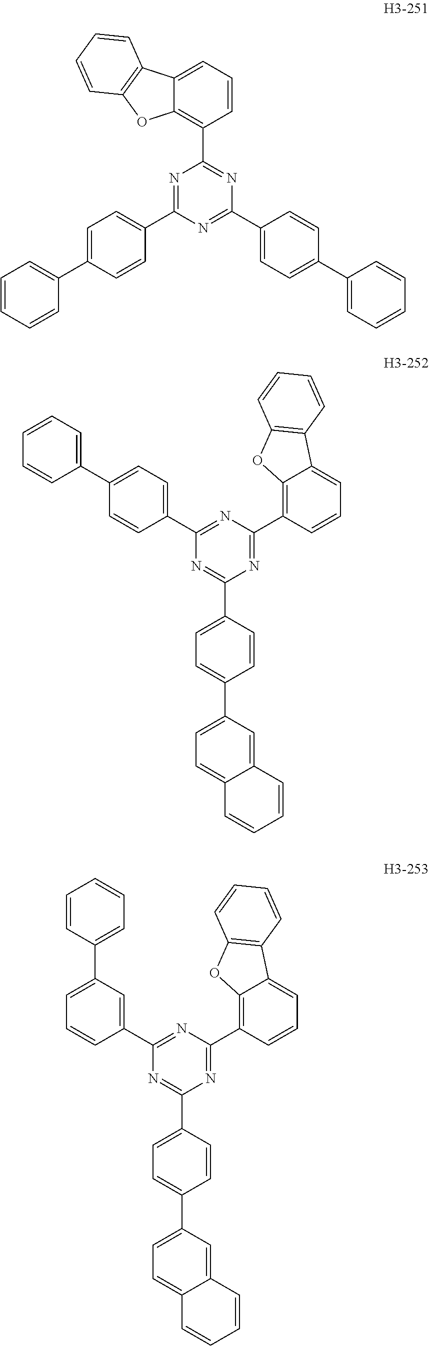 [SMILES: c1ccc(-c2ccc(-c3nc(-c4ccc(-c5ccccc5)cc4)nc(-c4cccc5c4oc4ccccc45)n3)cc2)cc1 ; c1ccc(-c2ccc(-c3nc(-c4ccc(-c5ccc6ccccc6c5)cc4)nc(-c4cccc5c4oc4ccccc45)n3)cc2)cc1 ; c1ccc(-c2cccc(-c3nc(-c4ccc(-c5ccc6ccccc6c5)cc4)nc(-c4cccc5c4oc4ccccc45)n3)c2)cc1]

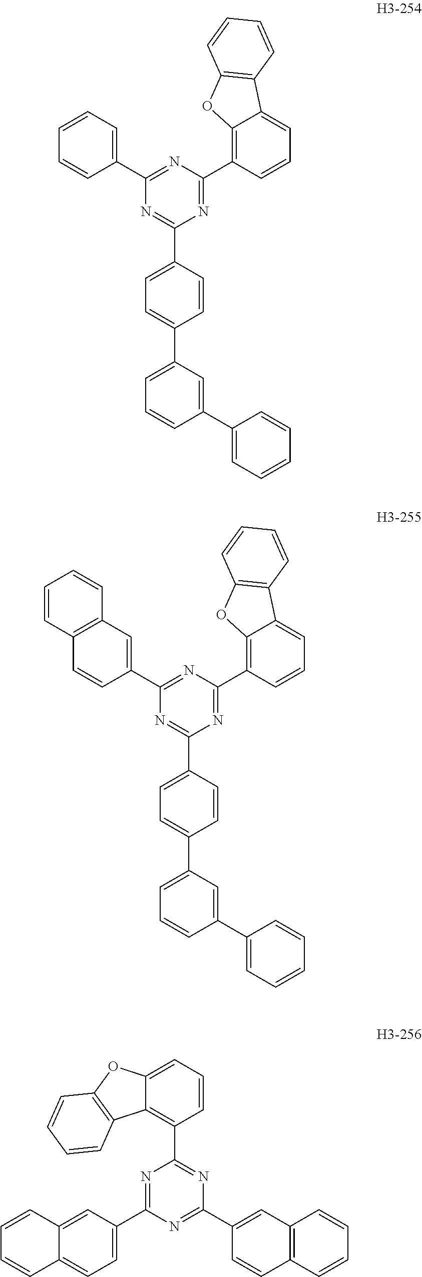 [SMILES: c1ccc(-c2cccc(-c3ccc(-c4nc(-c5ccccc5)nc(-c5cccc6c5oc5ccccc56)n4)cc3)c2)cc1 ; c1ccc(-c2cccc(-c3ccc(-c4nc(-c5ccc6ccccc6c5)nc(-c5cccc6c5oc5ccccc56)n4)cc3)c2)cc1 ; c1ccc2cc(-c3nc(-c4ccc5ccccc5c4)nc(-c4cccc5oc6ccccc6c45)n3)ccc2c1]

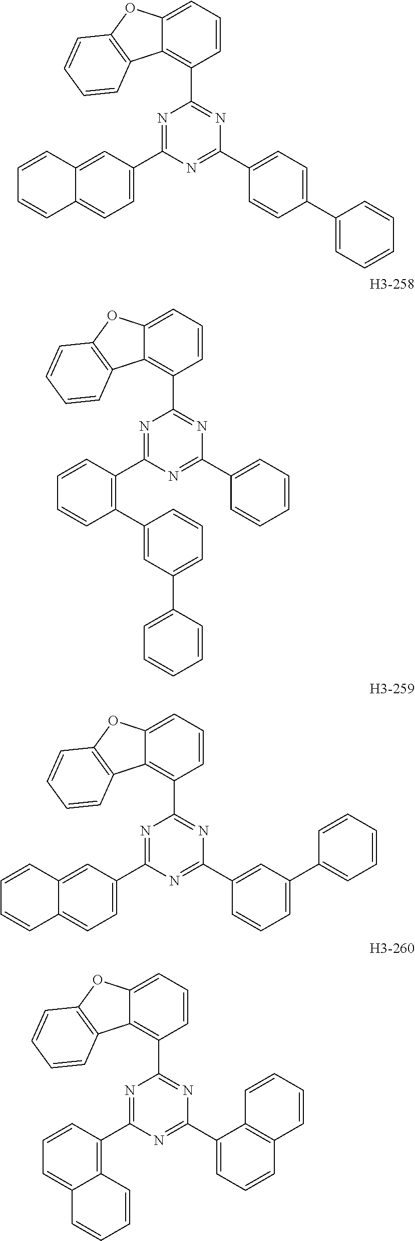

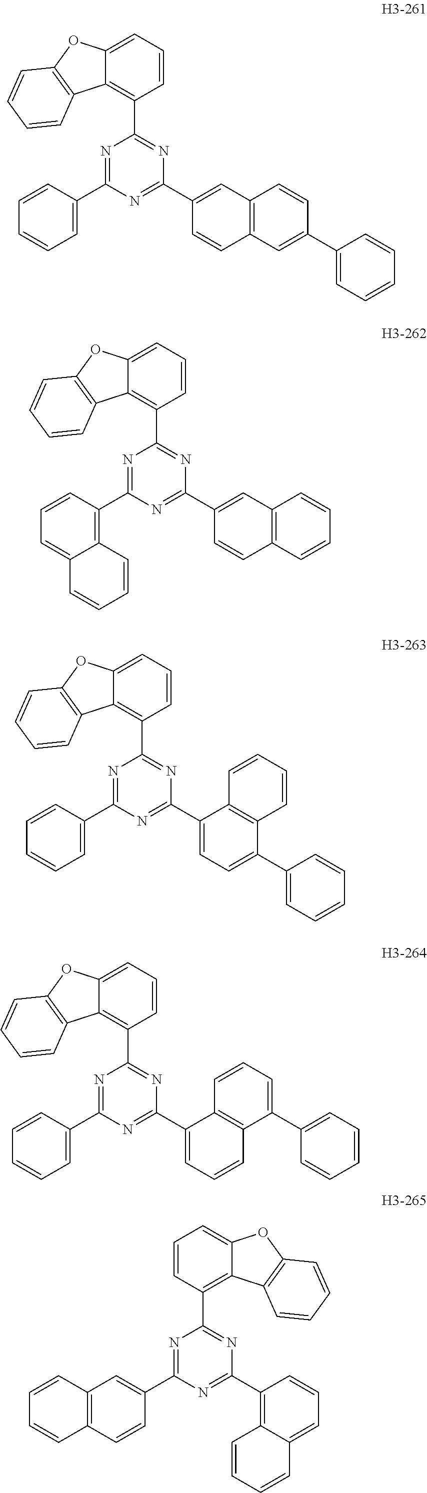 [SMILES: c1ccc(-c2ccc3cc(-c4nc(-c5ccccc5)nc(-c5cccc6oc7ccccc7c56)n4)ccc3c2)cc1 ; c1ccc2cc(-c3nc(-c4cccc5ccccc45)nc(-c4cccc5oc6ccccc6c45)n3)ccc2c1 ; c1ccc(-c2nc(-c3ccc(-c4ccccc4)c4ccccc34)nc(-c3cccc4oc5ccccc5c34)n2)cc1 ; c1ccc(-c2nc(-c3cccc4c(-c5ccccc5)cccc34)nc(-c3cccc4oc5ccccc5c34)n2)cc1 ; c1ccc2cc(-c3nc(-c4cccc5ccccc45)nc(-c4cccc5oc6ccccc6c45)n3)ccc2c1]

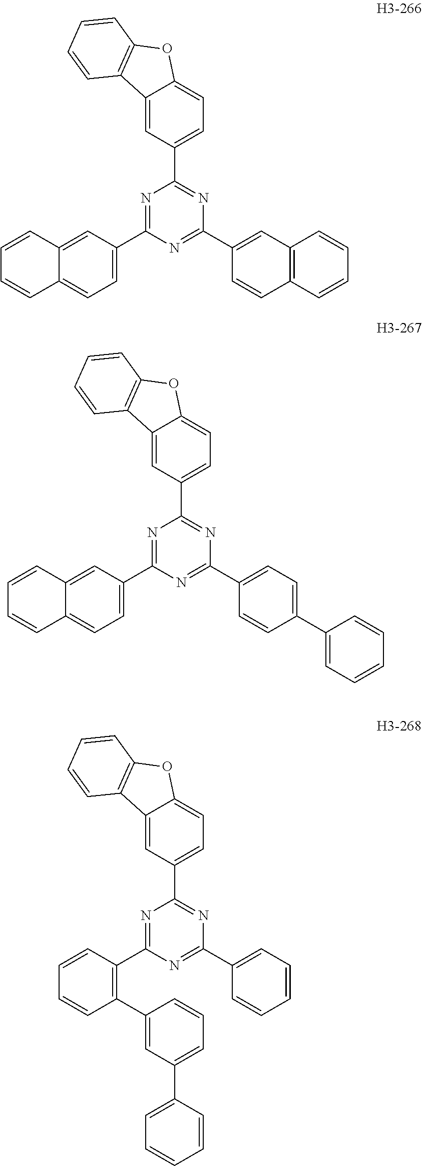 [SMILES: c1ccc2cc(-c3nc(-c4ccc5ccccc5c4)nc(-c4ccc5oc6ccccc6c5c4)n3)ccc2c1 ; c1ccc(-c2ccc(-c3nc(-c4ccc5ccccc5c4)nc(-c4ccc5oc6ccccc6c5c4)n3)cc2)cc1 ; c1ccc(-c2cccc(-c3ccccc3-c3nc(-c4ccccc4)nc(-c4ccc5oc6ccccc6c5c4)n3)c2)cc1]

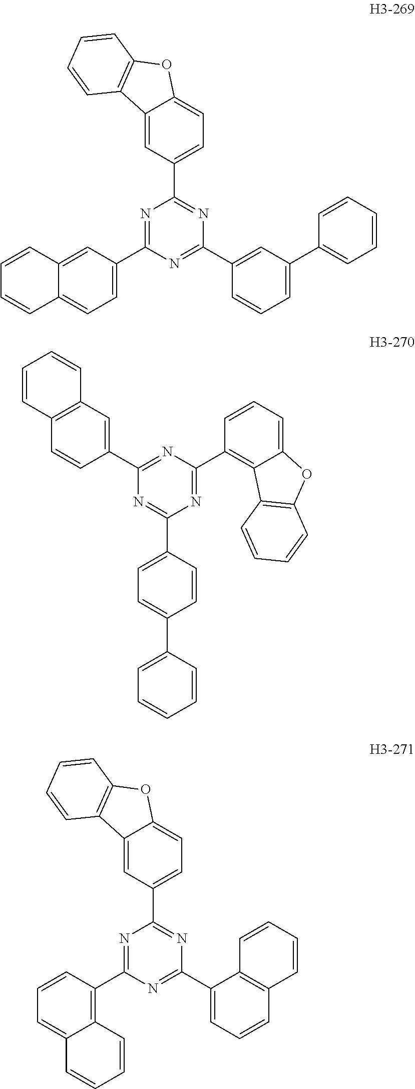

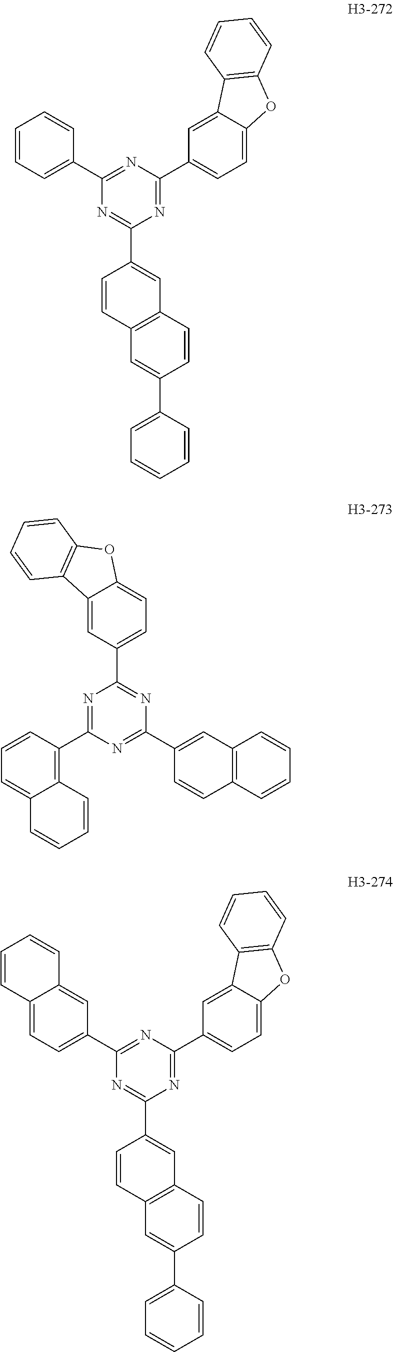 [SMILES: c1ccc(-c2ccc3cc(-c4nc(-c5ccccc5)nc(-c5ccc6oc7ccccc7c6c5)n4)ccc3c2)cc1 ; c1ccc2cc(-c3nc(-c4ccc5oc6ccccc6c5c4)nc(-c4cccc5ccccc45)n3)ccc2c1 ; c1ccc(-c2ccc3cc(-c4nc(-c5ccc6ccccc6c5)nc(-c5ccc6oc7ccccc7c6c5)n4)ccc3c2)cc1]

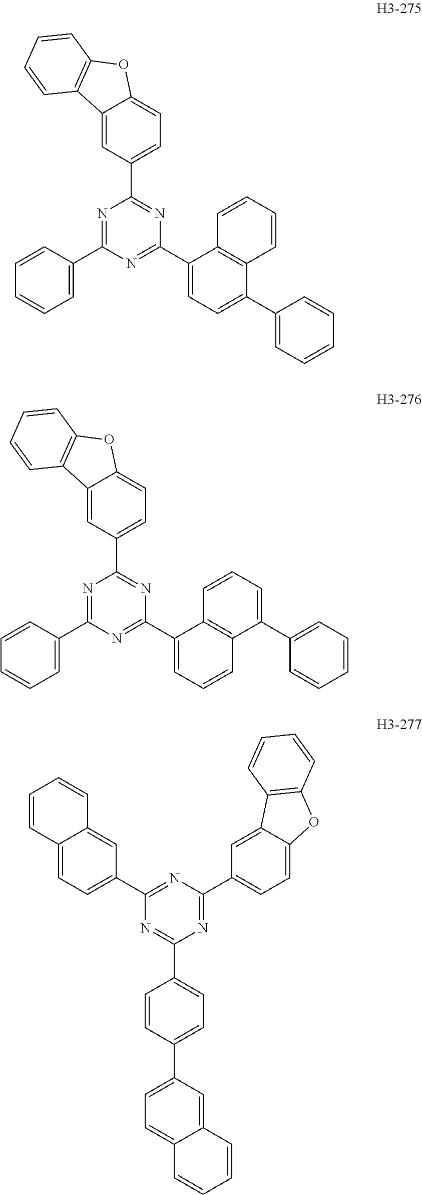 [SMILES: c1ccc(-c2nc(-c3ccc4oc5ccccc5c4c3)nc(-c3ccc(-c4ccccc4)c4ccccc34)n2)cc1 ; c1ccc(-c2nc(-c3ccc4oc5ccccc5c4c3)nc(-c3cccc4c(-c5ccccc5)cccc34)n2)cc1 ; c1ccc2cc(-c3ccc(-c4nc(-c5ccc6ccccc6c5)nc(-c5ccc6oc7ccccc7c6c5)n4)cc3)ccc2c1]

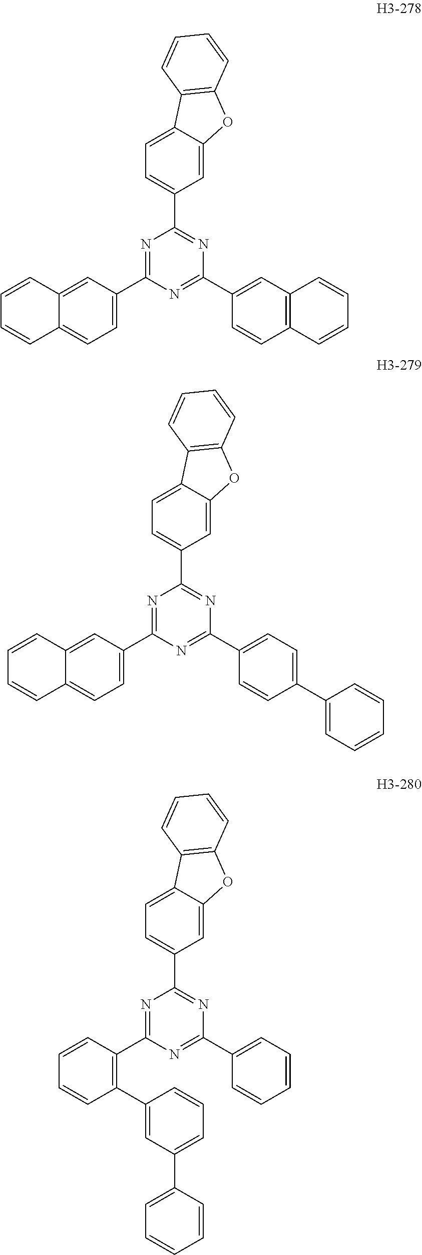 [SMILES: c1ccc2cc(-c3nc(-c4ccc5ccccc5c4)nc(-c4ccc5c(c4)oc4ccccc45)n3)ccc2c1 ; c1ccc(-c2ccc(-c3nc(-c4ccc5ccccc5c4)nc(-c4ccc5c(c4)oc4ccccc45)n3)cc2)cc1 ; c1ccc(-c2cccc(-c3ccccc3-c3nc(-c4ccccc4)nc(-c4ccc5c(c4)oc4ccccc45)n3)c2)cc1]

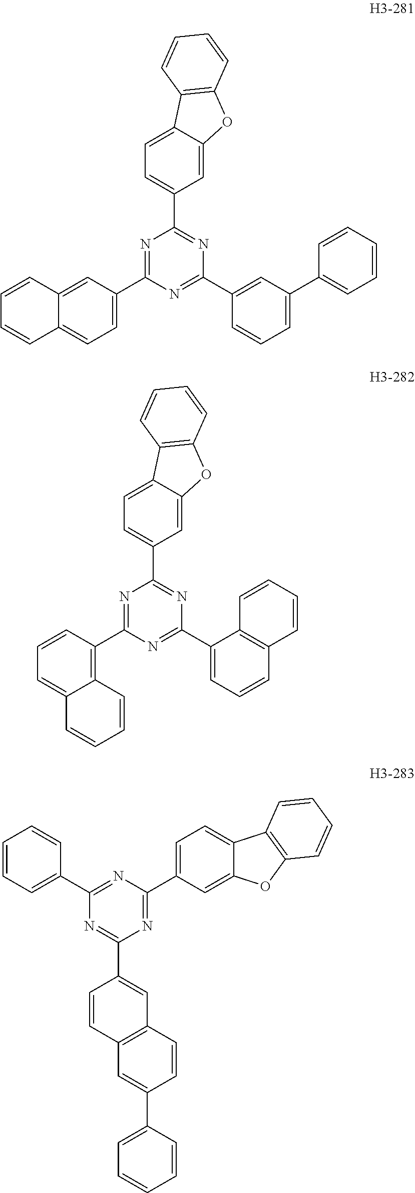 [SMILES: c1ccc(-c2cccc(-c3nc(-c4ccc5ccccc5c4)nc(-c4ccc5c(c4)oc4ccccc45)n3)c2)cc1 ; c1ccc2c(-c3nc(-c4ccc5c(c4)oc4ccccc45)nc(-c4cccc5ccccc45)n3)cccc2c1 ; c1ccc(-c2ccc3cc(-c4nc(-c5ccccc5)nc(-c5ccc6c(c5)oc5ccccc56)n4)ccc3c2)cc1]

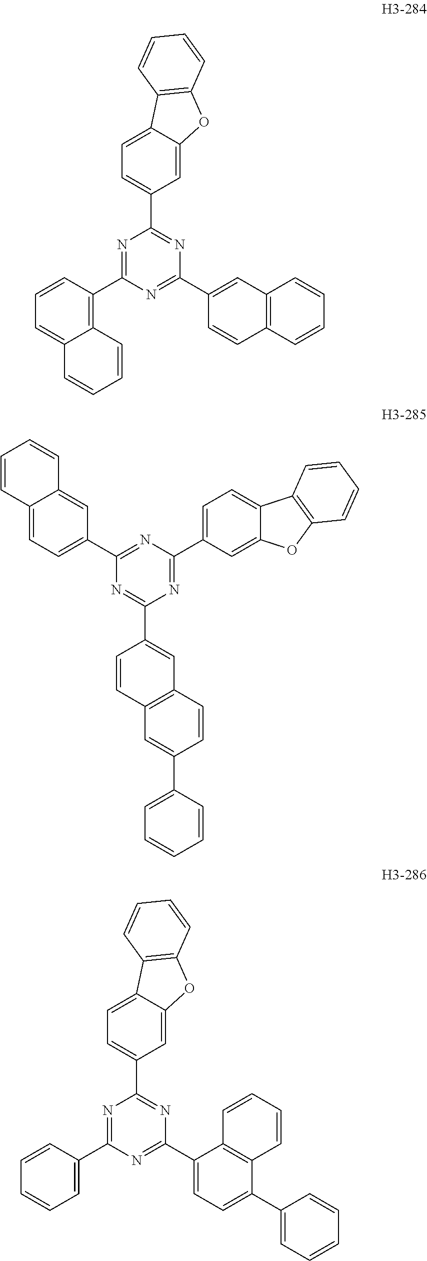

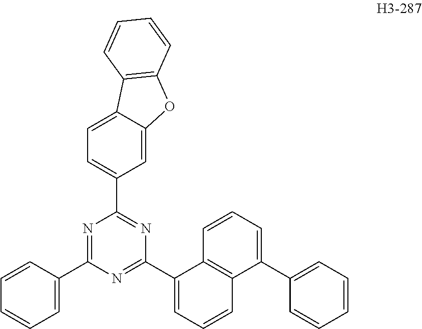 [SMILES: c1ccc(-c2nc(-c3ccc4c(c3)oc3ccccc34)nc(-c3cccc4c(-c5ccccc5)cccc34)n2)cc1]

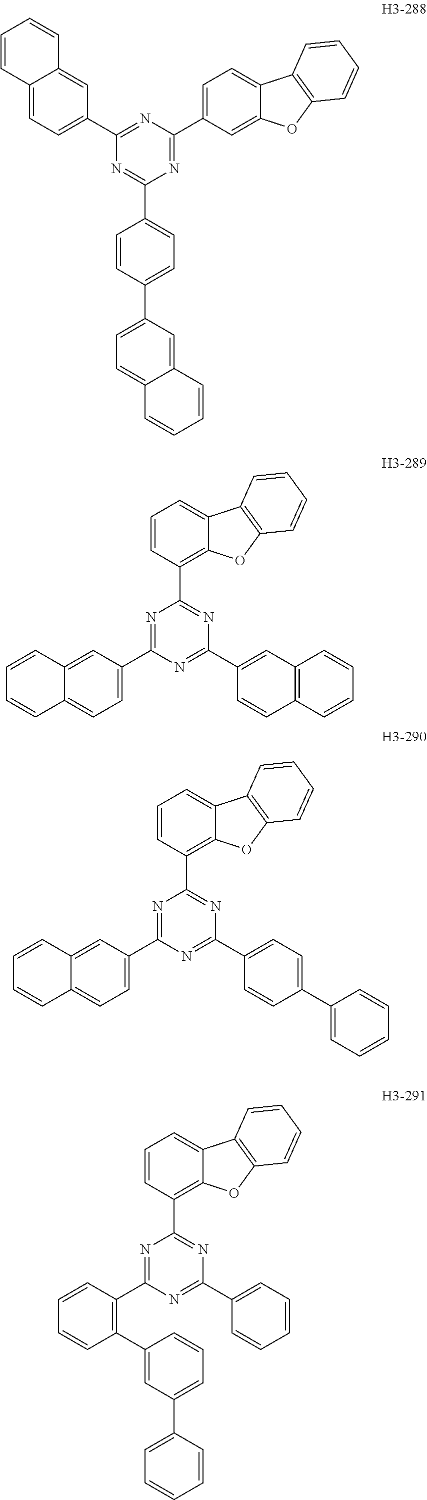 [SMILES: c1ccc2cc(-c3ccc(-c4nc(-c5ccc6ccccc6c5)nc(-c5ccc6c(c5)oc5ccccc56)n4)cc3)ccc2c1 ; c1ccc2cc(-c3nc(-c4ccc5ccccc5c4)nc(-c4cccc5c4oc4ccccc45)n3)ccc2c1 ; c1ccc(-c2ccc(-c3nc(-c4ccc5ccccc5c4)nc(-c4cccc5c4oc4ccccc45)n3)cc2)cc1 ; c1ccc(-c2cccc(-c3ccccc3-c3nc(-c4ccccc4)nc(-c4cccc5c4oc4ccccc45)n3)c2)cc1]

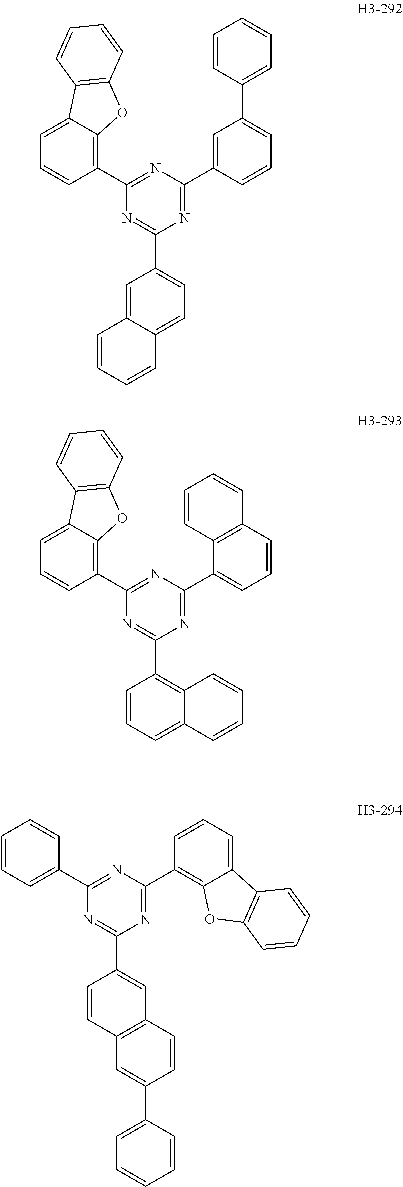 [SMILES: c1ccc(-c2cccc(-c3nc(-c4ccc5ccccc5c4)nc(-c4cccc5c4oc4ccccc45)n3)c2)cc1 ; c1ccc2c(-c3nc(-c4cccc5ccccc45)nc(-c4cccc5c4oc4ccccc45)n3)cccc2c1 ; c1ccc(-c2ccc3cc(-c4nc(-c5ccccc5)nc(-c5cccc6c5oc5ccccc56)n4)ccc3c2)cc1]

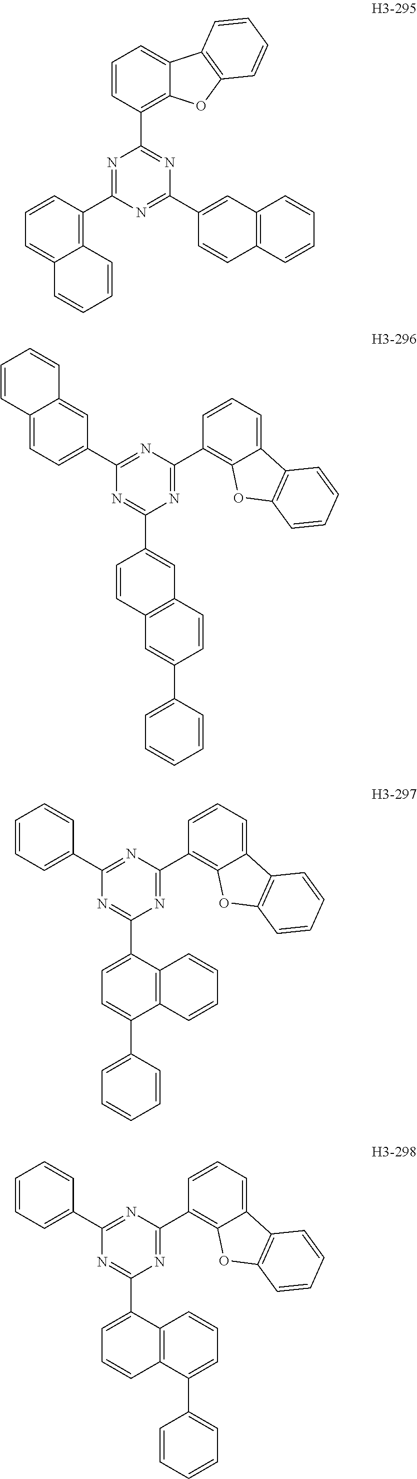 [SMILES: c1ccc2cc(-c3nc(-c4cccc5ccccc45)nc(-c4cccc5c4oc4ccccc45)n3)ccc2c1 ; c1ccc(-c2ccc3cc(-c4nc(-c5ccc6ccccc6c5)nc(-c5cccc6c5oc5ccccc56)n4)ccc3c2)cc1 ; c1ccc(-c2nc(-c3ccc(-c4ccccc4)c4ccccc34)nc(-c3cccc4c3oc3ccccc34)n2)cc1 ; c1ccc(-c2nc(-c3cccc4c(-c5ccccc5)cccc34)nc(-c3cccc4c3oc3ccccc34)n2)cc1]

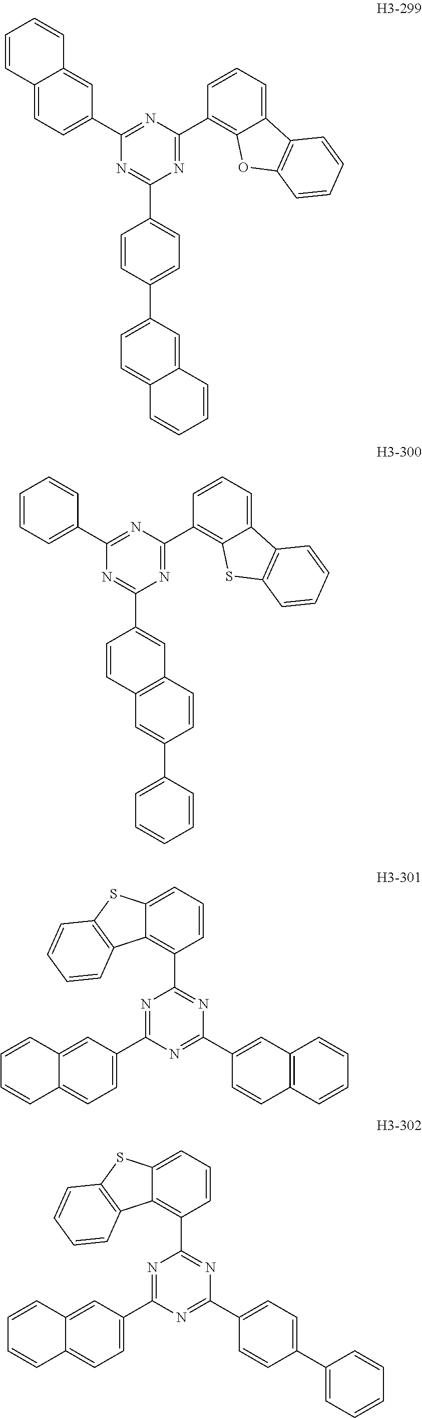

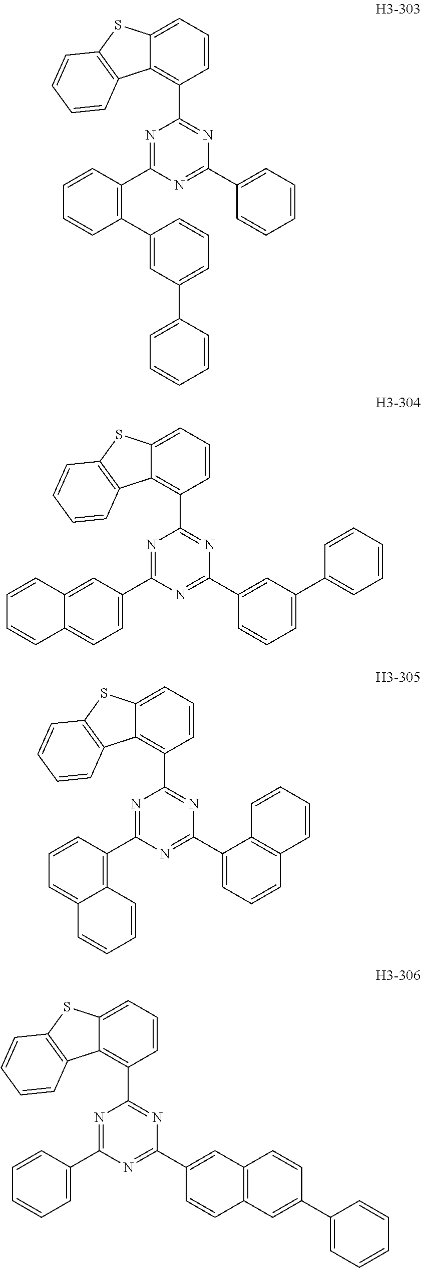

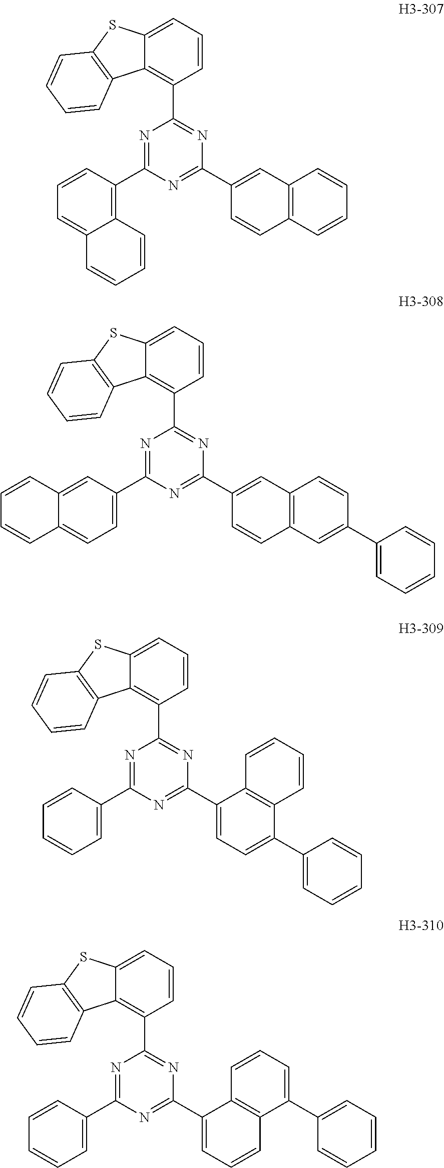 [SMILES: c1ccc2cc(-c3nc(-c4cccc5ccccc45)nc(-c4cccc5sc6ccccc6c45)n3)ccc2c1 ; c1ccc(-c2ccc3cc(-c4nc(-c5ccc6ccccc6c5)nc(-c5cccc6sc7ccccc7c56)n4)ccc3c2)cc1 ; c1ccc(-c2nc(-c3ccc(-c4ccccc4)c4ccccc34)nc(-c3cccc4sc5ccccc5c34)n2)cc1 ; c1ccc(-c2nc(-c3cccc4c(-c5ccccc5)cccc34)nc(-c3cccc4sc5ccccc5c34)n2)cc1]

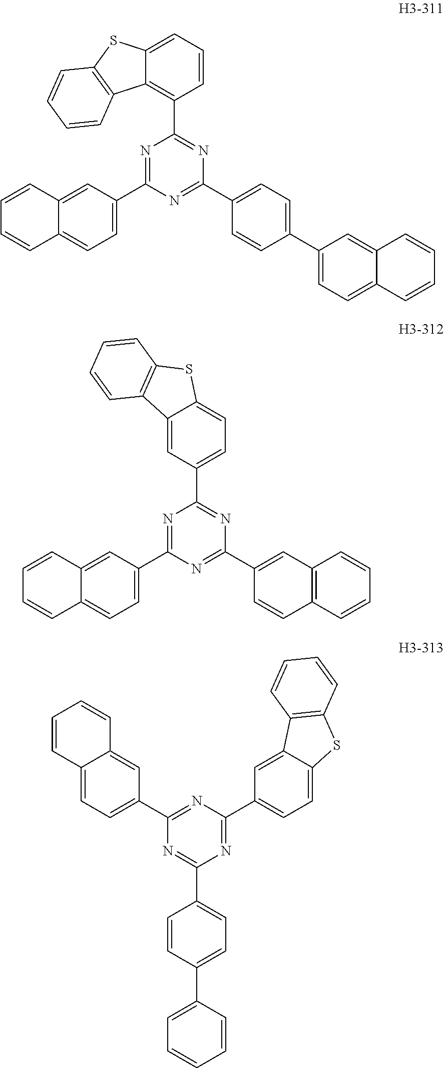

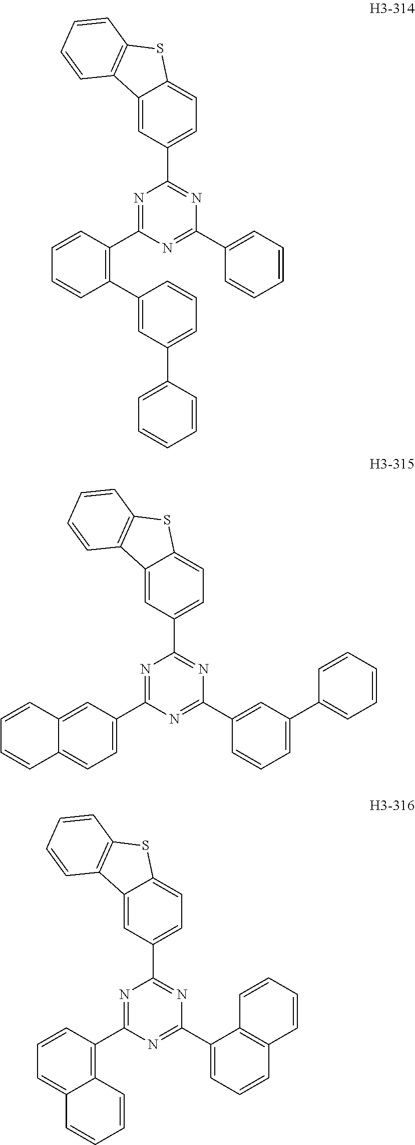

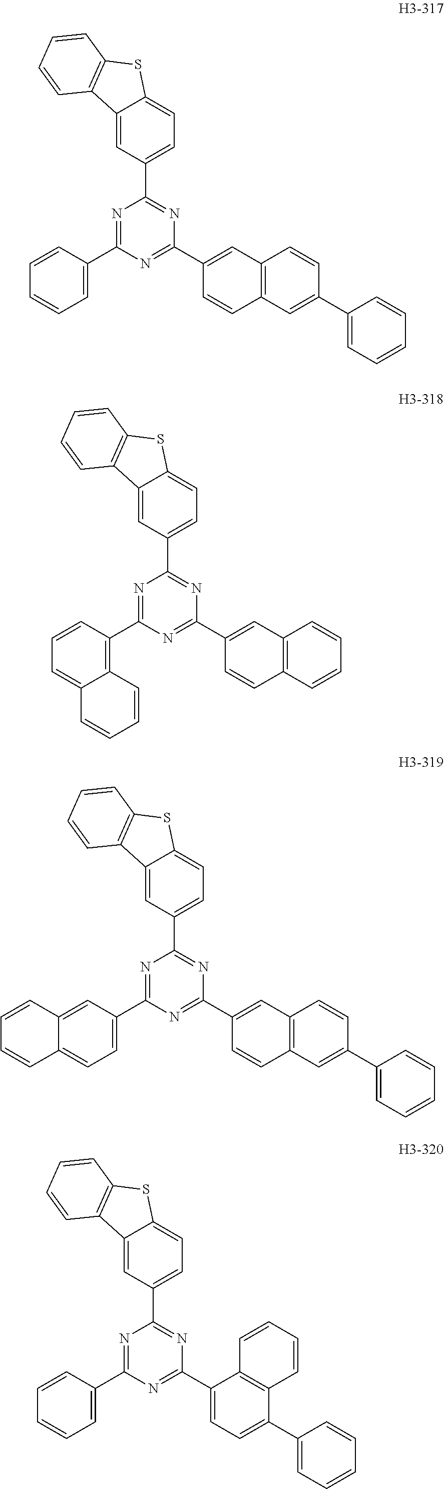 [SMILES: c1ccc(-c2ccc3cc(-c4nc(-c5ccccc5)nc(-c5ccc6sc7ccccc7c6c5)n4)ccc3c2)cc1 ; c1ccc2cc(-c3nc(-c4ccc5sc6ccccc6c5c4)nc(-c4cccc5ccccc45)n3)ccc2c1 ; c1ccc(-c2ccc3cc(-c4nc(-c5ccc6ccccc6c5)nc(-c5ccc6sc7ccccc7c6c5)n4)ccc3c2)cc1 ; c1ccc(-c2nc(-c3ccc4sc5ccccc5c4c3)nc(-c3ccc(-c4ccccc4)c4ccccc34)n2)cc1]

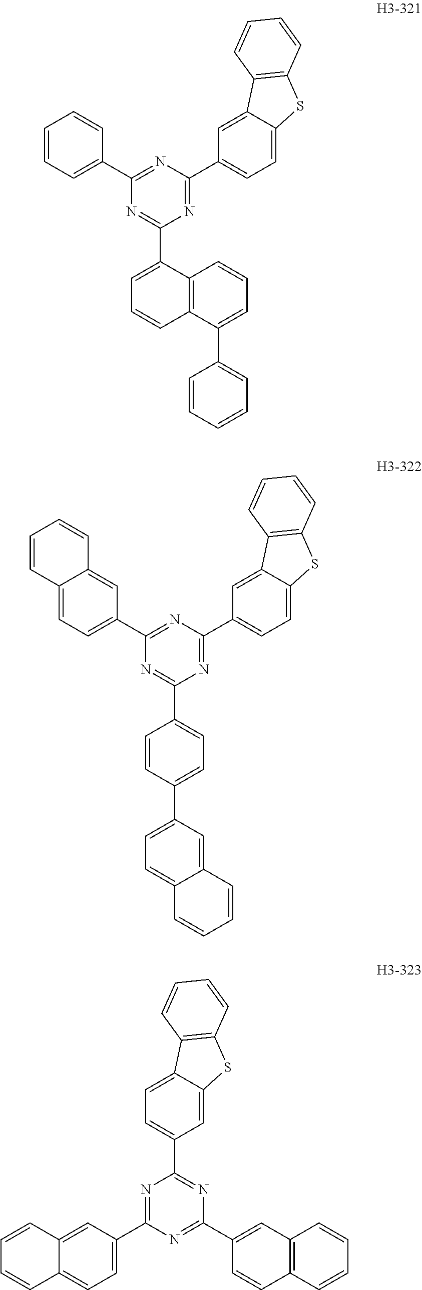

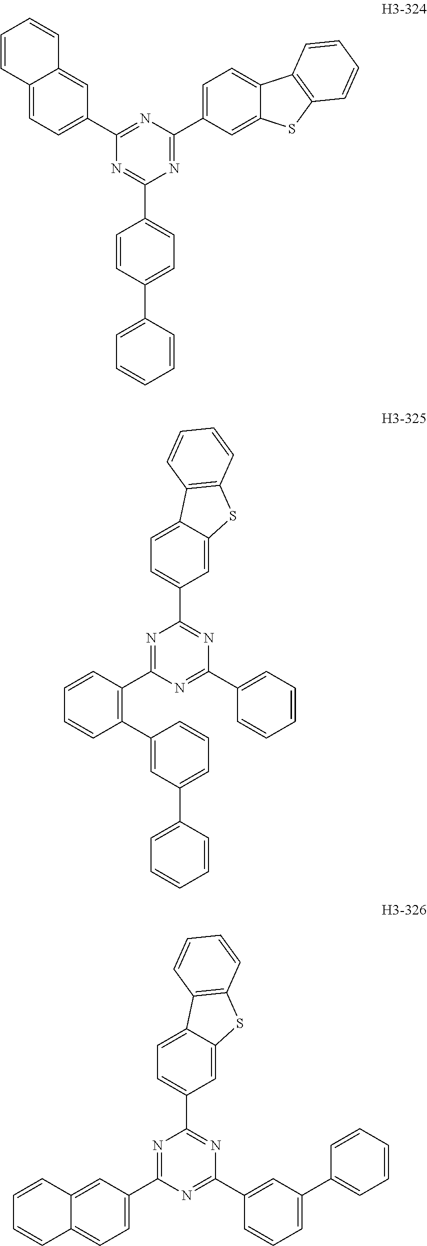 [SMILES: c1ccc(-c2ccc(-c3nc(-c4ccc5ccccc5c4)nc(-c4ccc5c(c4)sc4ccccc45)n3)cc2)cc1 ; c1ccc(-c2cccc(-c3ccccc3-c3nc(-c4ccccc4)nc(-c4ccc5c(c4)sc4ccccc45)n3)c2)cc1 ; c1ccc(-c2cccc(-c3nc(-c4ccc5ccccc5c4)nc(-c4ccc5c(c4)sc4ccccc45)n3)c2)cc1]

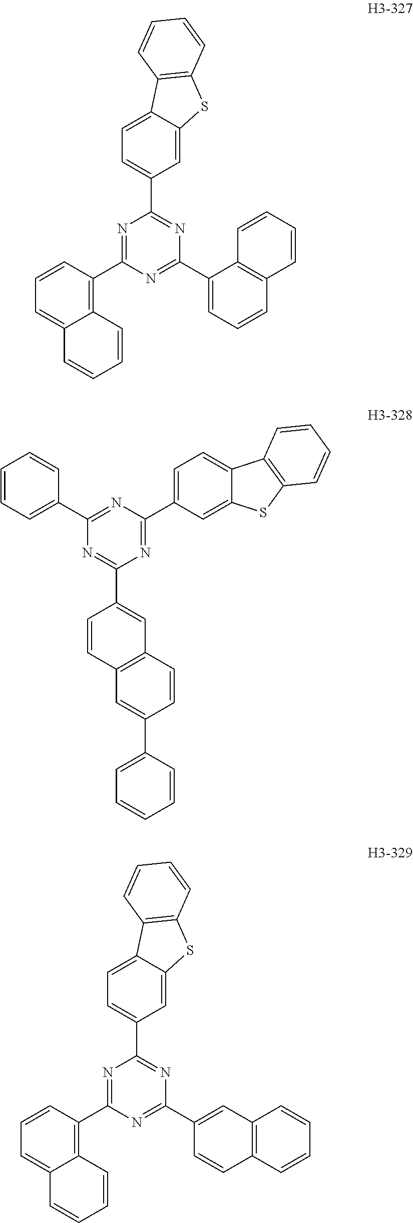 [SMILES: c1ccc2c(-c3nc(-c4ccc5c(c4)sc4ccccc45)nc(-c4cccc5ccccc45)n3)cccc2c1 ; c1ccc(-c2ccc3cc(-c4nc(-c5ccccc5)nc(-c5ccc6c(c5)sc5ccccc56)n4)ccc3c2)cc1 ; c1ccc2cc(-c3nc(-c4ccc5c(c4)sc4ccccc45)nc(-c4cccc5ccccc45)n3)ccc2c1]

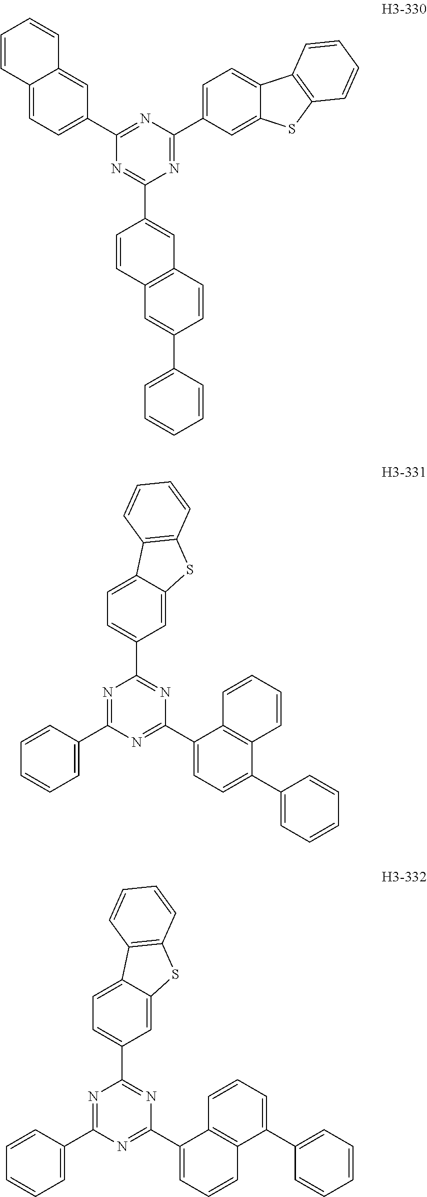 [SMILES: c1ccc(-c2ccc3cc(-c4nc(-c5ccc6ccccc6c5)nc(-c5ccc6c(c5)sc5ccccc56)n4)ccc3c2)cc1 ; c1ccc(-c2nc(-c3ccc4c(c3)sc3ccccc34)nc(-c3ccc(-c4ccccc4)c4ccccc34)n2)cc1 ; c1ccc(-c2nc(-c3ccc4c(c3)sc3ccccc34)nc(-c3cccc4c(-c5ccccc5)cccc34)n2)cc1]

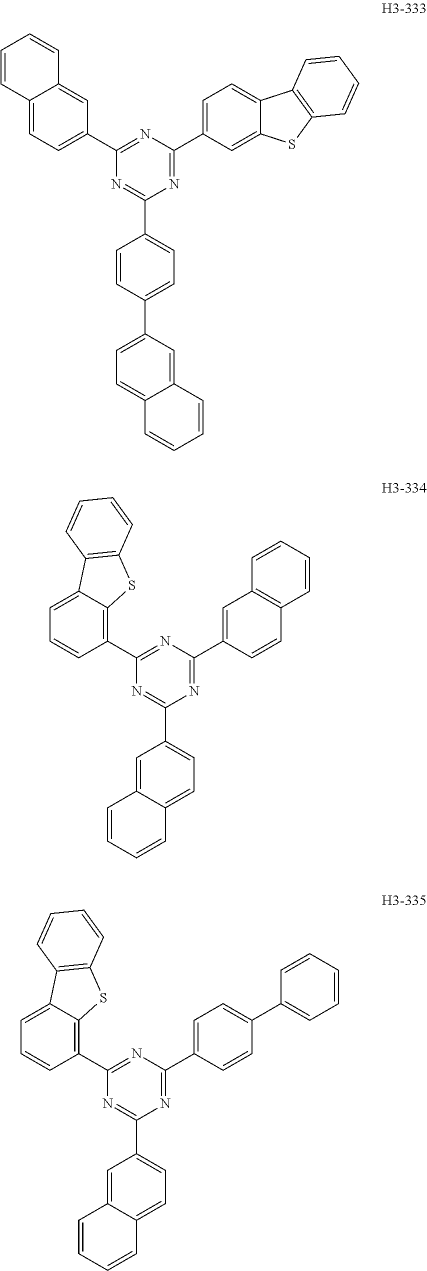 [SMILES: c1ccc2cc(-c3ccc(-c4nc(-c5ccc6ccccc6c5)nc(-c5ccc6c(c5)sc5ccccc56)n4)cc3)ccc2c1 ; c1ccc2cc(-c3nc(-c4ccc5ccccc5c4)nc(-c4cccc5c4sc4ccccc45)n3)ccc2c1 ; c1ccc(-c2ccc(-c3nc(-c4ccc5ccccc5c4)nc(-c4cccc5c4sc4ccccc45)n3)cc2)cc1]

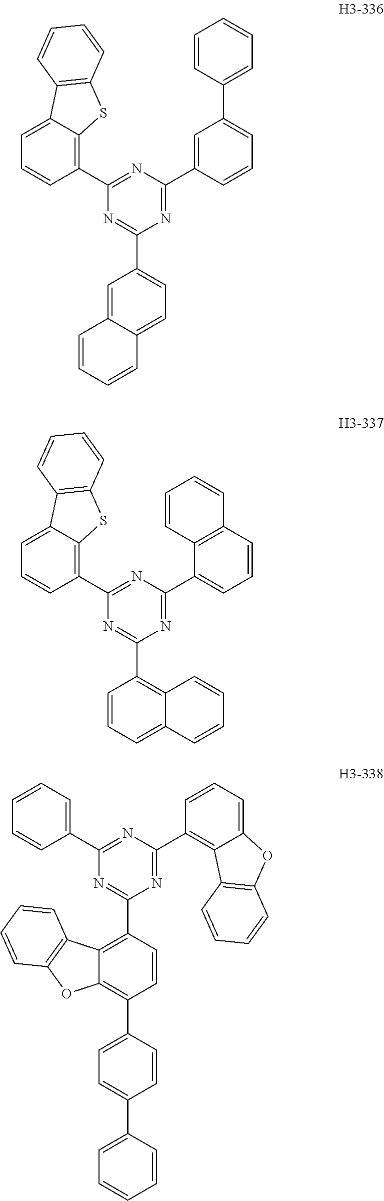

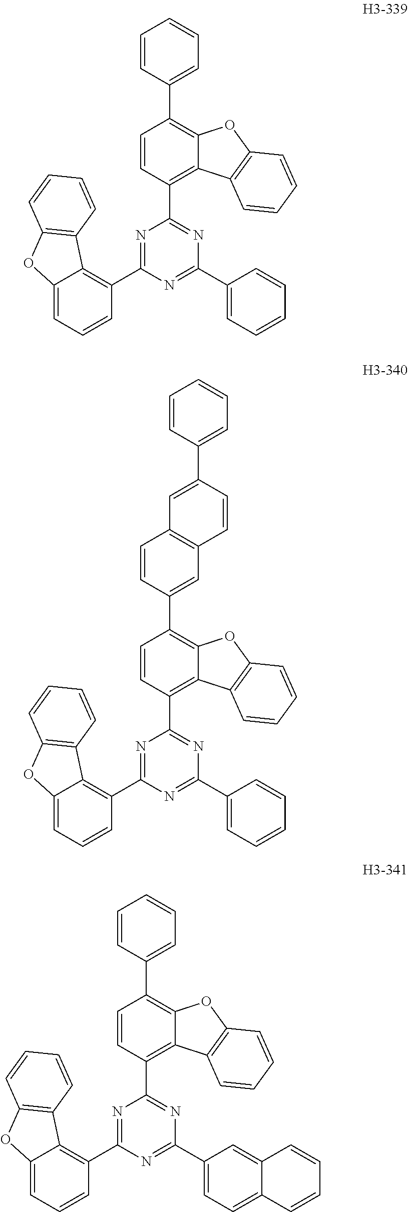 [SMILES: c1ccc(-c2nc(-c3cccc4oc5ccccc5c34)nc(-c3ccc(-c4ccccc4)c4oc5ccccc5c34)n2)cc1 ; c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc(-c2ccc(-c3nc(-c4ccc5ccccc5c4)nc(-c4cccc5oc6ccccc6c45)n3)c3c2oc2ccccc23)cc1]

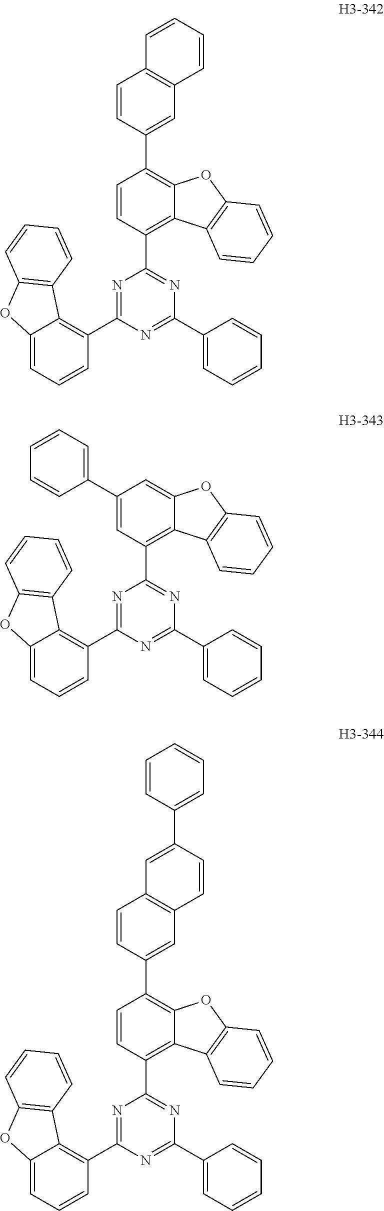

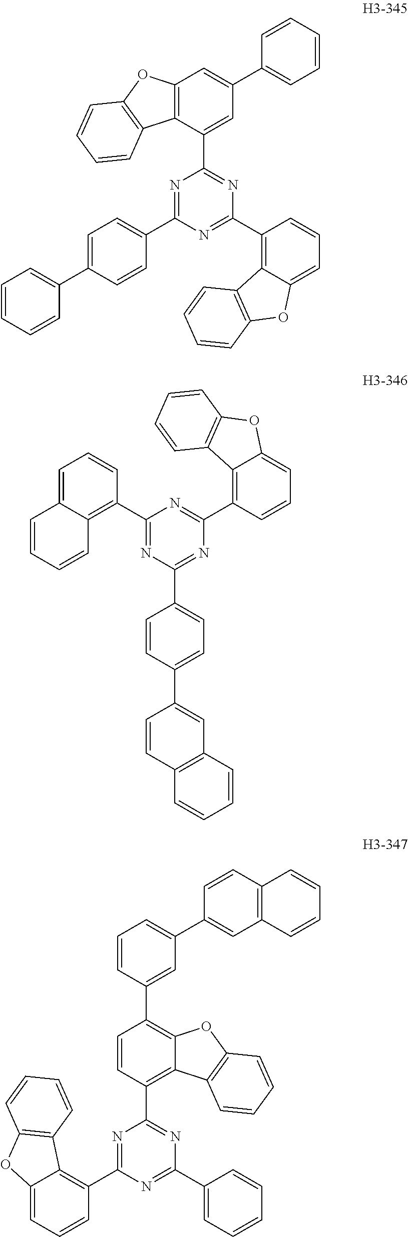

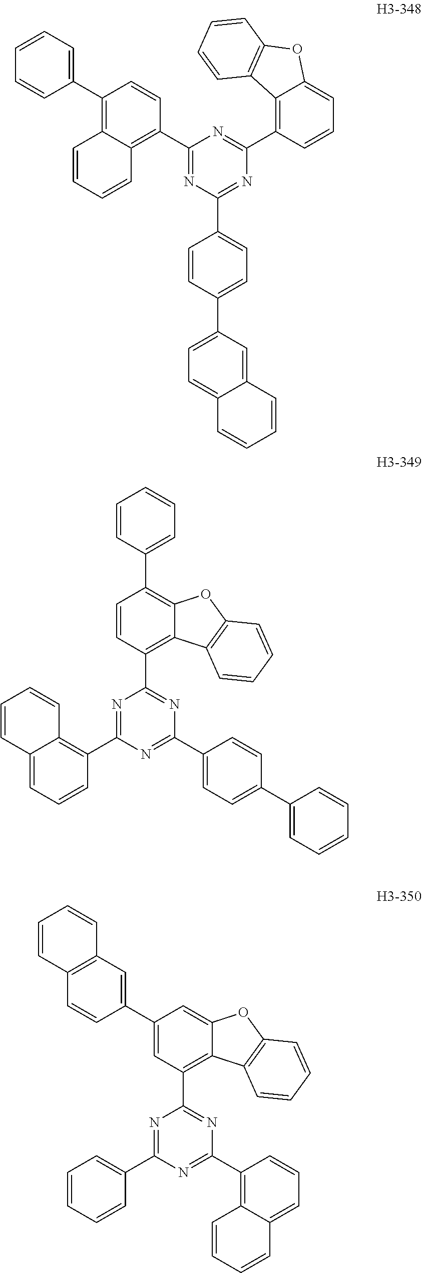 [SMILES: c1ccc(-c2ccc(-c3nc(-c4ccc(-c5ccc6ccccc6c5)cc4)nc(-c4cccc5oc6ccccc6c45)n3)c3ccccc23)cc1 ; c1ccc(-c2ccc(-c3nc(-c4cccc5ccccc45)nc(-c4ccc(-c5ccccc5)c5oc6ccccc6c45)n3)cc2)cc1 ; c1ccc(-c2nc(-c3cccc4ccccc34)nc(-c3cc(-c4ccc5ccccc5c4)cc4oc5ccccc5c34)n2)cc1]

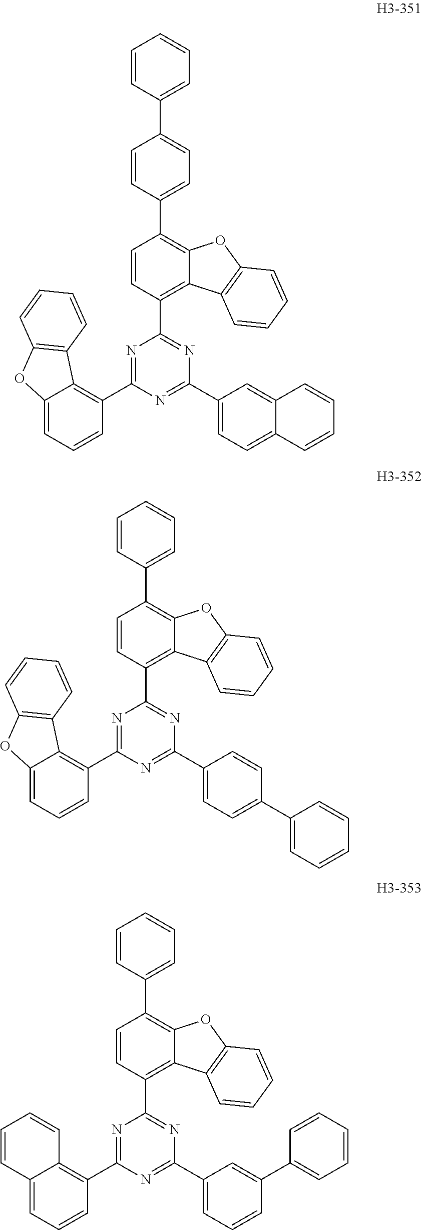 [SMILES: c1ccc(-c2ccc(-c3ccc(-c4nc(-c5ccc6ccccc6c5)nc(-c5cccc6oc7ccccc7c56)n4)c4c3oc3ccccc34)cc2)cc1 ; c1ccc(-c2ccc(-c3nc(-c4cccc5oc6ccccc6c45)nc(-c4ccc(-c5ccccc5)c5oc6ccccc6c45)n3)cc2)cc1 ; c1ccc(-c2cccc(-c3nc(-c4cccc5ccccc45)nc(-c4ccc(-c5ccccc5)c5oc6ccccc6c45)n3)c2)cc1]

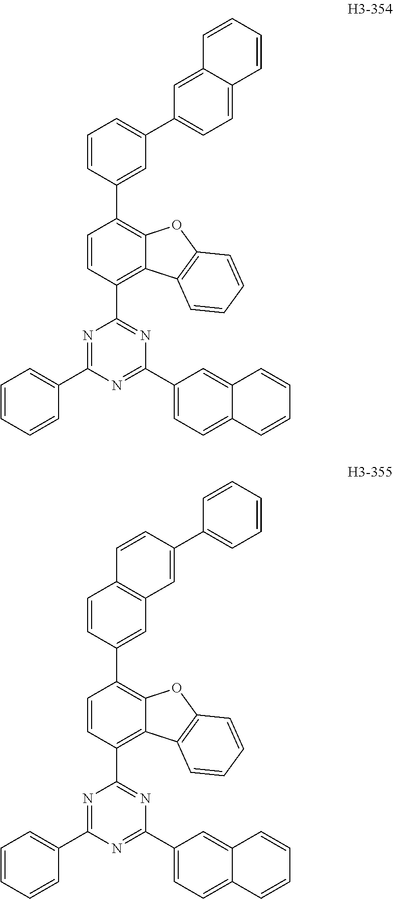 [SMILES: c1ccc(-c2nc(-c3ccc4ccccc4c3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccc7ccccc7c6)n5)c5c4oc4ccccc45)cc3c2)cc1]

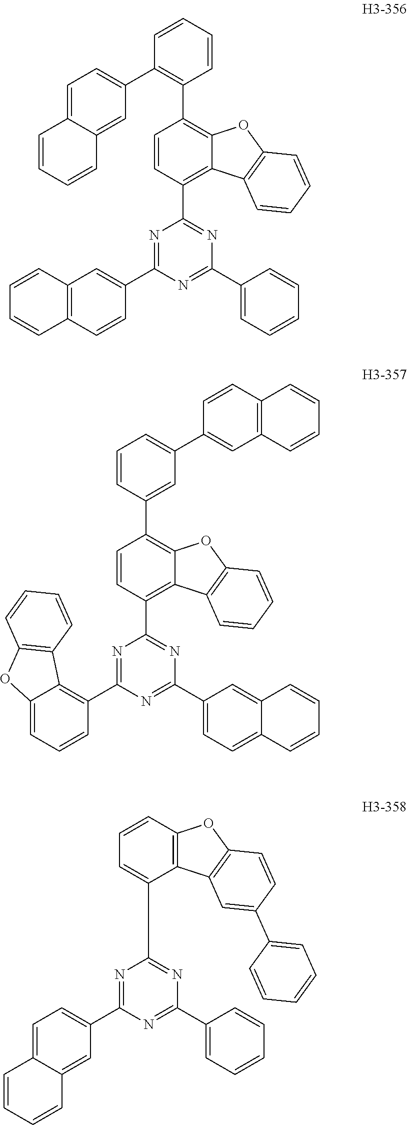 [SMILES: c1ccc(-c2nc(-c3ccc4ccccc4c3)nc(-c3ccc(-c4ccccc4-c4ccc5ccccc5c4)c4oc5ccccc5c34)n2)cc1 ; c1cc(-c2ccc3ccccc3c2)cc(-c2ccc(-c3nc(-c4ccc5ccccc5c4)nc(-c4cccc5oc6ccccc6c45)n3)c3c2oc2ccccc23)c1 ; c1ccc(-c2ccc3oc4cccc(-c5nc(-c6ccccc6)nc(-c6ccc7ccccc7c6)n5)c4c3c2)cc1]

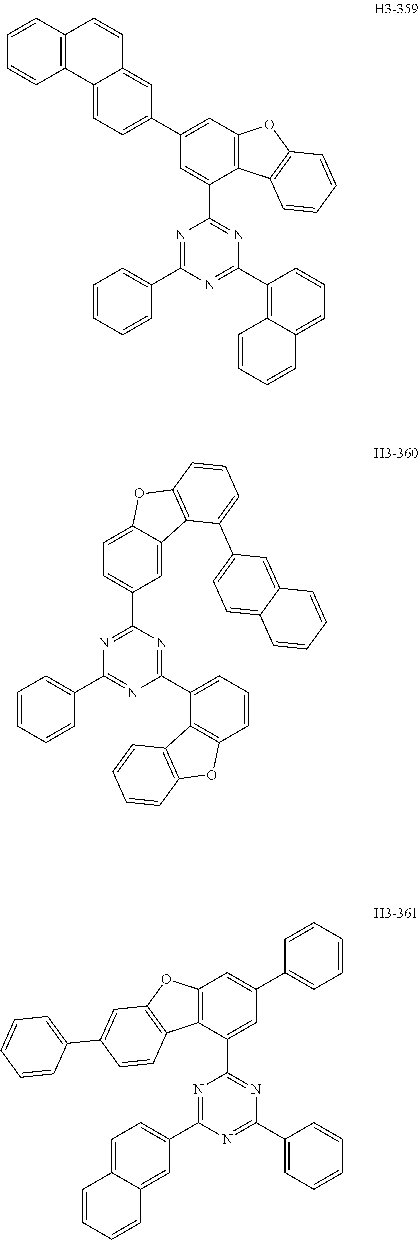 [SMILES: c1ccc(-c2nc(-c3cccc4ccccc34)nc(-c3cc(-c4ccc5c(ccc6ccccc65)c4)cc4oc5ccccc5c34)n2)cc1 ; c1ccc(-c2nc(-c3ccc4oc5cccc(-c6ccc7ccccc7c6)c5c4c3)nc(-c3cccc4oc5ccccc5c34)n2)cc1 ; c1ccc(-c2ccc3c(c2)oc2cc(-c4ccccc4)cc(-c4nc(-c5ccccc5)nc(-c5ccc6ccccc6c5)n4)c23)cc1]

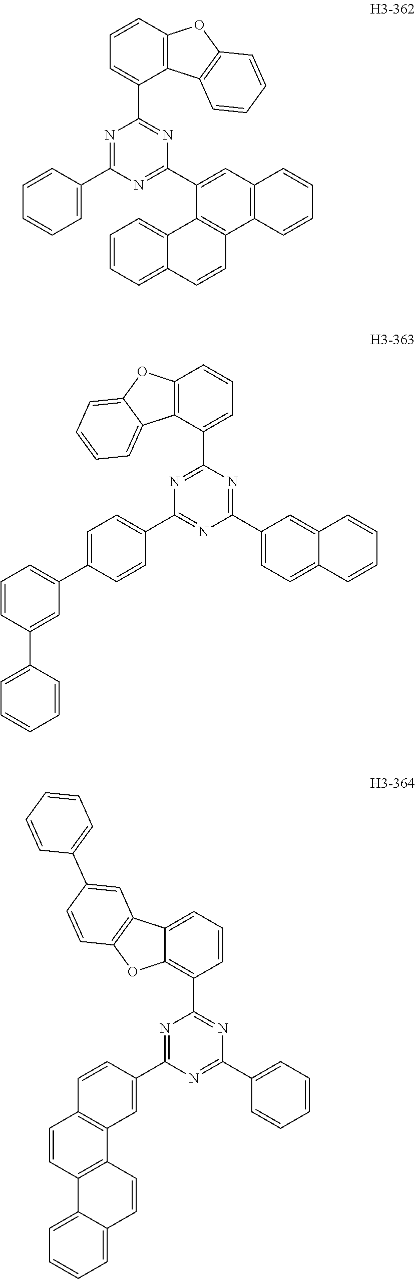 [SMILES: c1ccc(-c2nc(-c3cccc4oc5ccccc5c34)nc(-c3cc4ccccc4c4ccc5ccccc5c34)n2)cc1 ; c1ccc(-c2cccc(-c3ccc(-c4nc(-c5ccc6ccccc6c5)nc(-c5cccc6oc7ccccc7c56)n4)cc3)c2)cc1 ; c1ccc(-c2ccc3oc4c(-c5nc(-c6ccccc6)nc(-c6ccc7ccc8c9ccccc9ccc8c7c6)n5)cccc4c3c2)cc1]

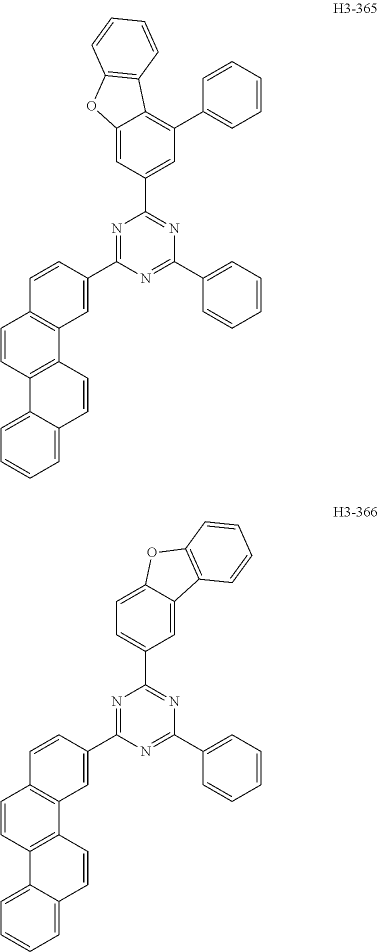 [SMILES: c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

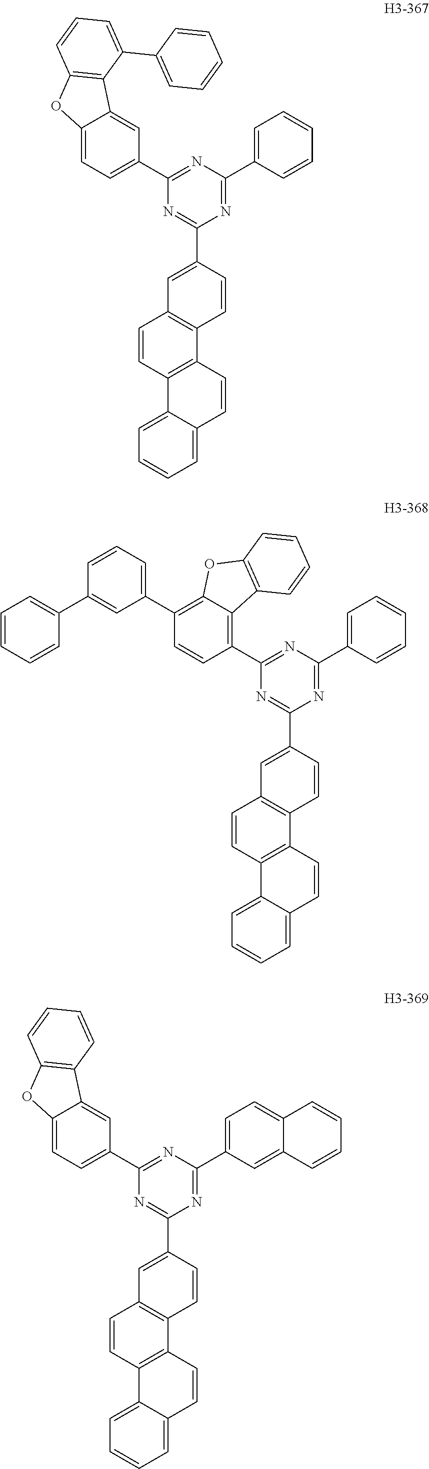 [SMILES: c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4ccc5oc6ccccc6c5c4)n3)ccc2c1]

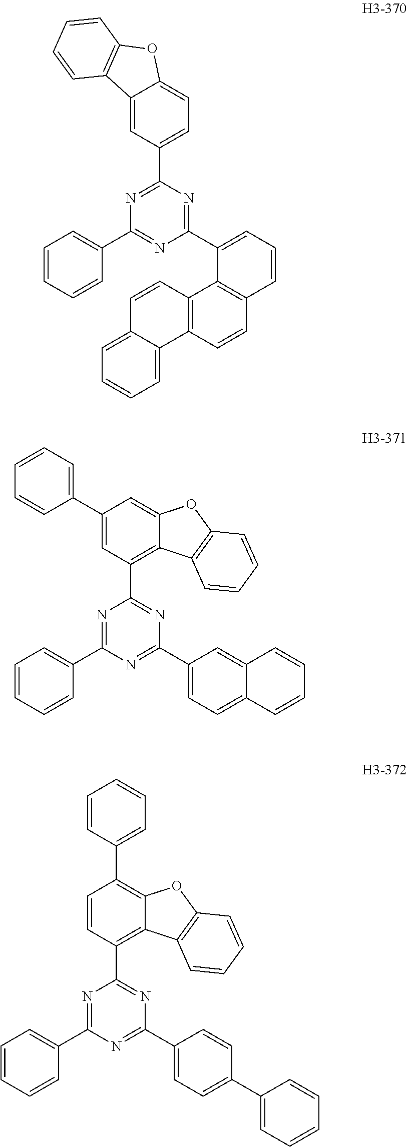 [SMILES: c1ccc(-c2nc(-c3ccc4oc5ccccc5c4c3)nc(-c3cccc4ccc5c6ccccc6ccc5c34)n2)cc1 ; c1ccc(-c2cc(-c3nc(-c4ccccc4)nc(-c4ccc5ccccc5c4)n3)c3c(c2)oc2ccccc23)cc1 ; c1ccc(-c2ccc(-c3nc(-c4ccccc4)nc(-c4ccc(-c5ccccc5)c5oc6ccccc6c45)n3)cc2)cc1]

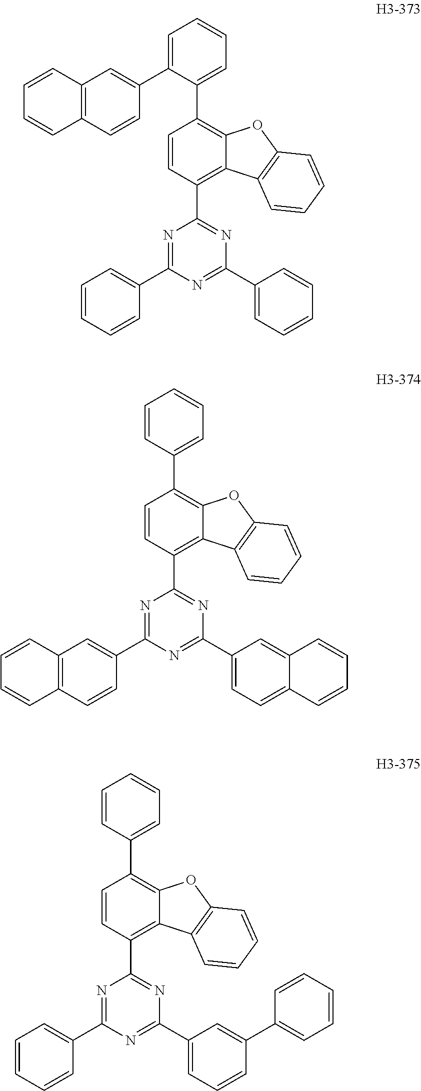 [SMILES: c1ccc(-c2nc(-c3ccccc3)nc(-c3ccc(-c4ccccc4-c4ccc5ccccc5c4)c4oc5ccccc5c34)n2)cc1 ; c1ccc(-c2ccc(-c3nc(-c4ccc5ccccc5c4)nc(-c4ccc5ccccc5c4)n3)c3c2oc2ccccc23)cc1 ; c1ccc(-c2cccc(-c3nc(-c4ccccc4)nc(-c4ccc(-c5ccccc5)c5oc6ccccc6c45)n3)c2)cc1]

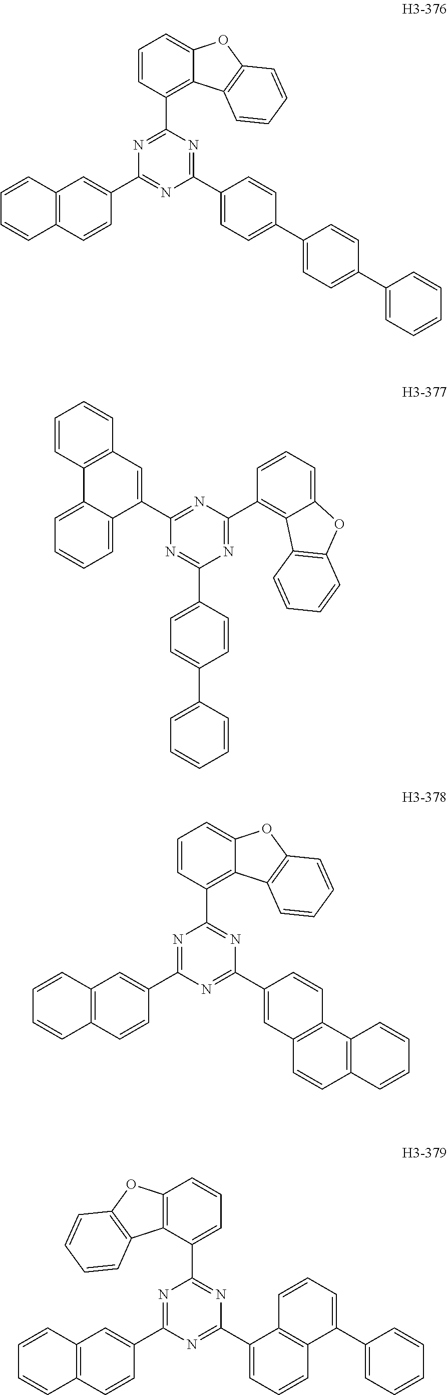 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3nc(-c4cc5ccccc5c5ccccc45)nc(-c4cccc5oc6ccccc6c45)n3)cc2)cc1 ; c1ccc2cc(-c3nc(-c4ccc5c(ccc6ccccc65)c4)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1]

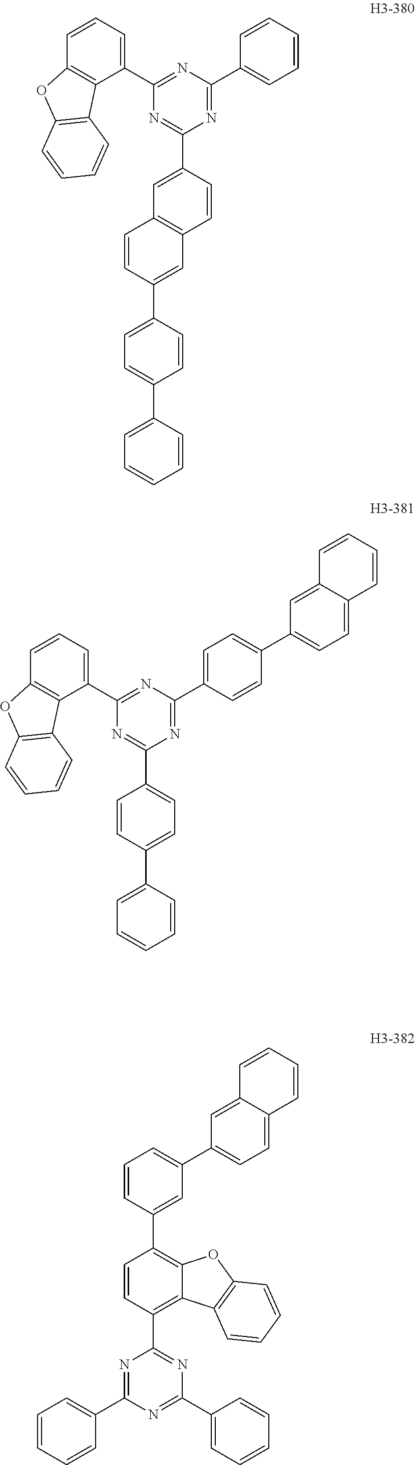 [SMILES: c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)cc2)cc1 ; c1ccc(-c2nc(-c3ccccc3)nc(-c3ccc(-c4cccc(-c5ccc6ccccc6c5)c4)c4oc5ccccc5c34)n2)cc1]

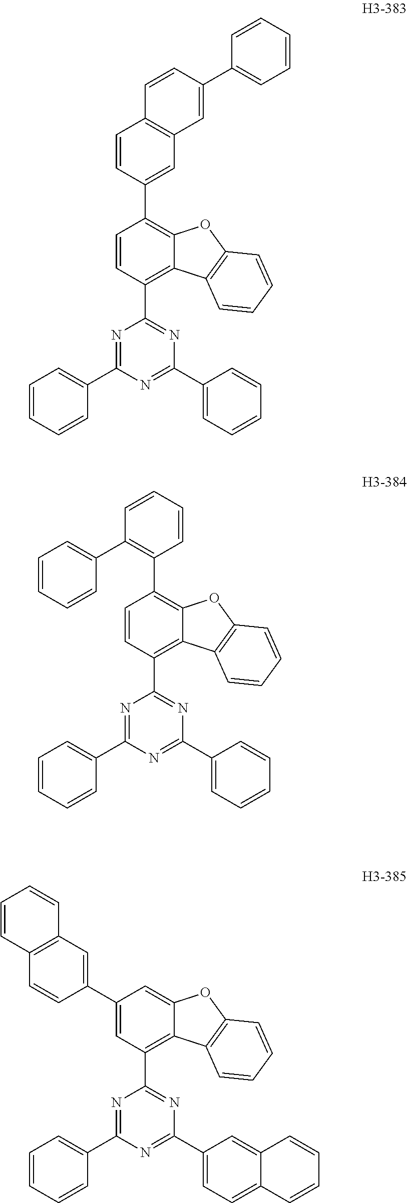 [SMILES: c1ccc(-c2ccc3ccc(-c4ccc(-c5nc(-c6ccccc6)nc(-c6ccccc6)n5)c5c4oc4ccccc45)cc3c2)cc1 ; c1ccc(-c2nc(-c3ccccc3)nc(-c3ccc(-c4ccccc4-c4ccccc4)c4oc5ccccc5c34)n2)cc1 ; c1ccc(-c2nc(-c3ccc4ccccc4c3)nc(-c3cc(-c4ccc5ccccc5c4)cc4oc5ccccc5c34)n2)cc1]

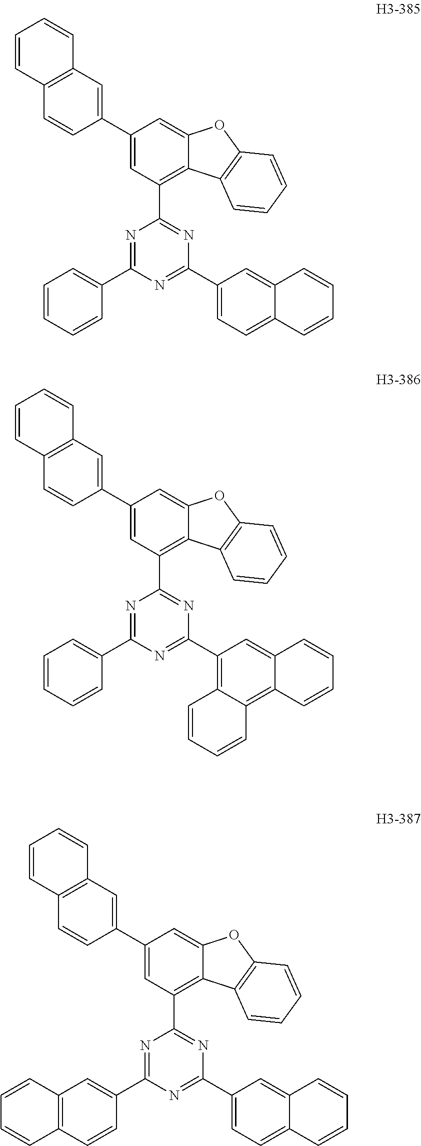 [SMILES: c1ccc(-c2nc(-c3ccc4ccccc4c3)nc(-c3cc(-c4ccc5ccccc5c4)cc4oc5ccccc5c34)n2)cc1 ; c1ccc(-c2nc(-c3cc4ccccc4c4ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5ccc6ccccc6c5)n4)c4c(c3)oc3ccccc34)ccc2c1]

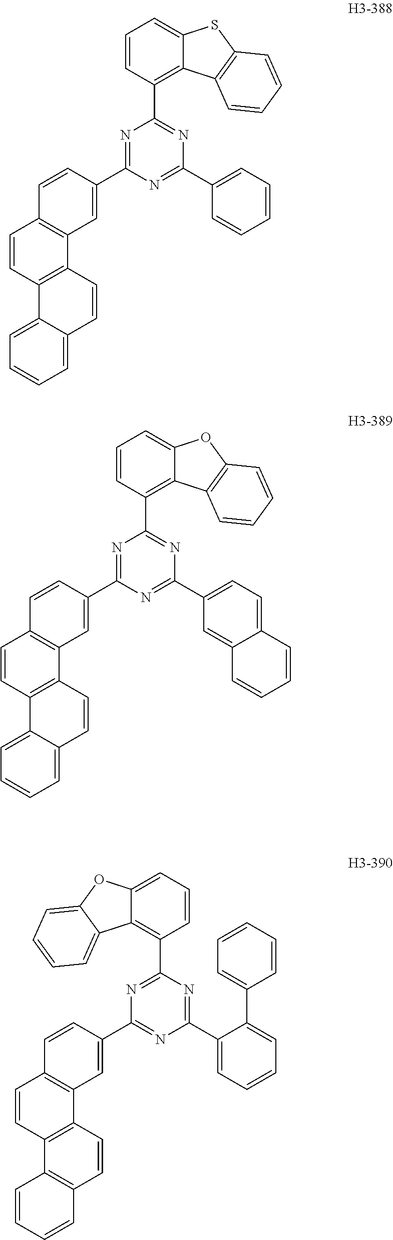 [SMILES: c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5oc6ccccc6c45)n3)ccc2c1 ; c1ccc(-c2ccccc2-c2nc(-c3ccc4ccc5c6ccccc6ccc5c4c3)nc(-c3cccc4oc5ccccc5c34)n2)cc1]

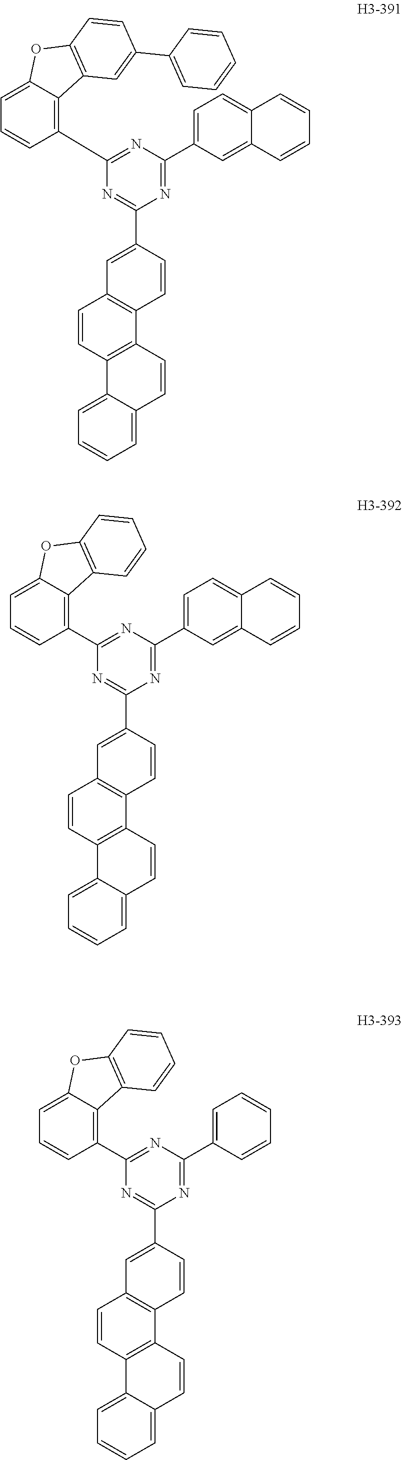 [SMILES: c1ccc(-c2ccc3oc4cccc(-c5nc(-c6ccc7ccccc7c6)nc(-c6ccc7c(ccc8c9ccccc9ccc78)c6)n5)c4c3c2)cc1 ; c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4oc5ccccc5c34)n2)cc1]

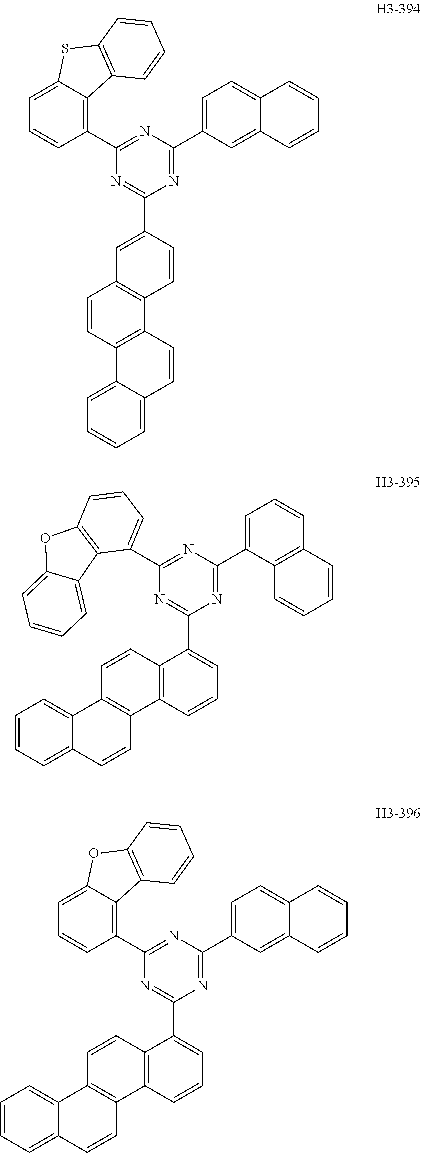

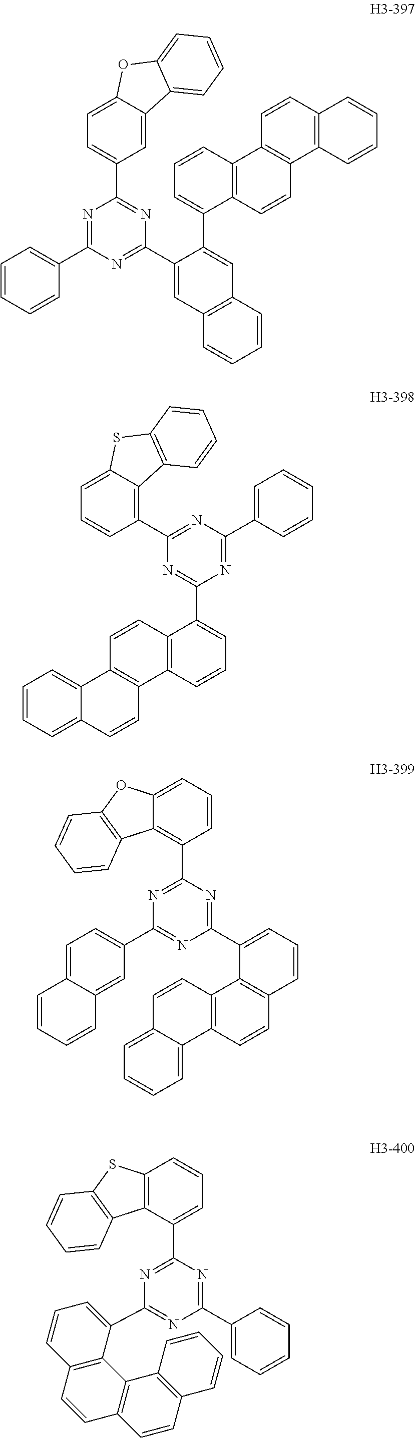 [SMILES: c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4sc5ccccc5c34)n2)cc1 ; c1ccc2cc(-c3nc(-c4cccc5ccc6c7ccccc7ccc6c45)nc(-c4cccc5oc6ccccc6c45)n3)ccc2c1 ; c1ccc(-c2nc(-c3cccc4sc5ccccc5c34)nc(-c3cccc4ccc5ccc6ccccc6c5c34)n2)cc1]

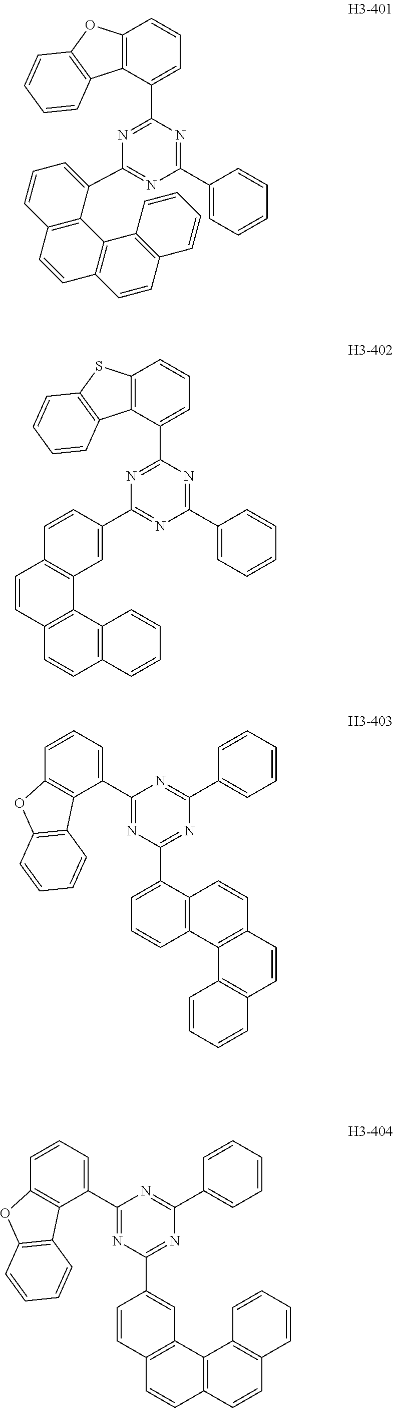 [SMILES: c1ccc(-c2nc(-c3cccc4oc5ccccc5c34)nc(-c3cccc4ccc5ccc6ccccc6c5c34)n2)cc1 ; c1ccc(-c2nc(-c3ccc4ccc5ccc6ccccc6c5c4c3)nc(-c3cccc4sc5ccccc5c34)n2)cc1 ; c1ccc(-c2nc(-c3cccc4c3ccc3ccc5ccccc5c34)nc(-c3cccc4oc5ccccc5c34)n2)cc1 ; c1ccc(-c2nc(-c3ccc4ccc5ccc6ccccc6c5c4c3)nc(-c3cccc4oc5ccccc5c34)n2)cc1]

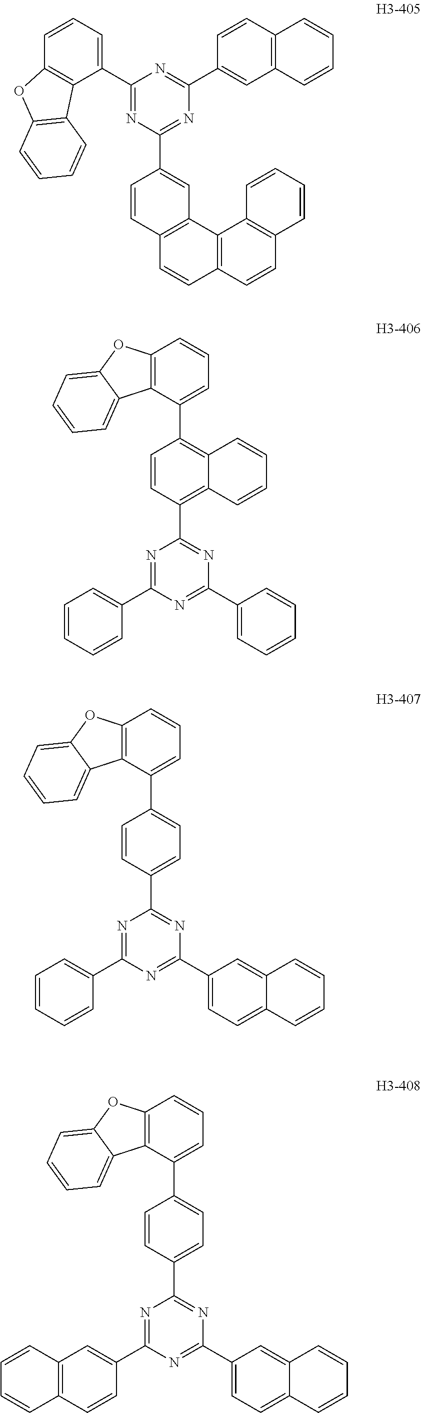 [SMILES: c1ccc2cc(-c3nc(-c4ccc5ccc6ccc7ccccc7c6c5c4)nc(-c4cccc5oc6ccccc6c45)n3)ccc2c1 ; c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1 ; c1ccc2cc(-c3nc(-c4ccc(-c5cccc6oc7ccccc7c56)cc4)nc(-c4ccc5ccccc5c4)n3)ccc2c1]

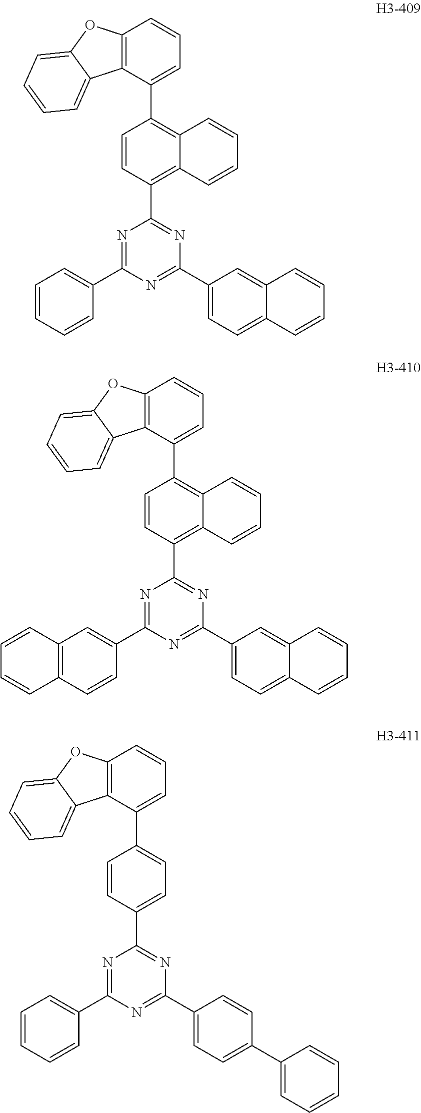 [SMILES: c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1 ; c1ccc(-c2ccc(-c3nc(-c4ccccc4)nc(-c4ccc(-c5cccc6oc7ccccc7c56)cc4)n3)cc2)cc1]

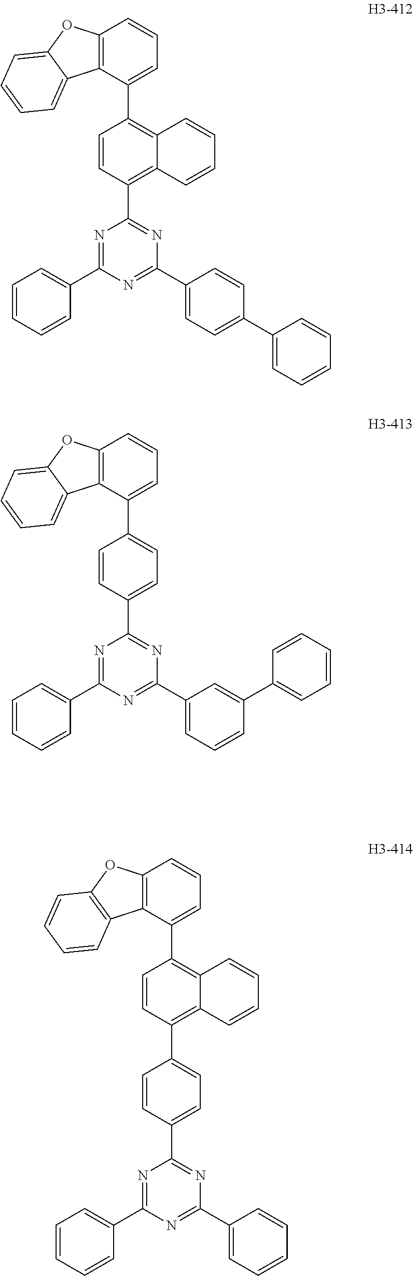 [SMILES: c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1 ; c1ccc(-c2nc(-c3ccccc3)nc(-c3ccc(-c4ccc(-c5cccc6oc7ccccc7c56)c5ccccc45)cc3)n2)cc1]

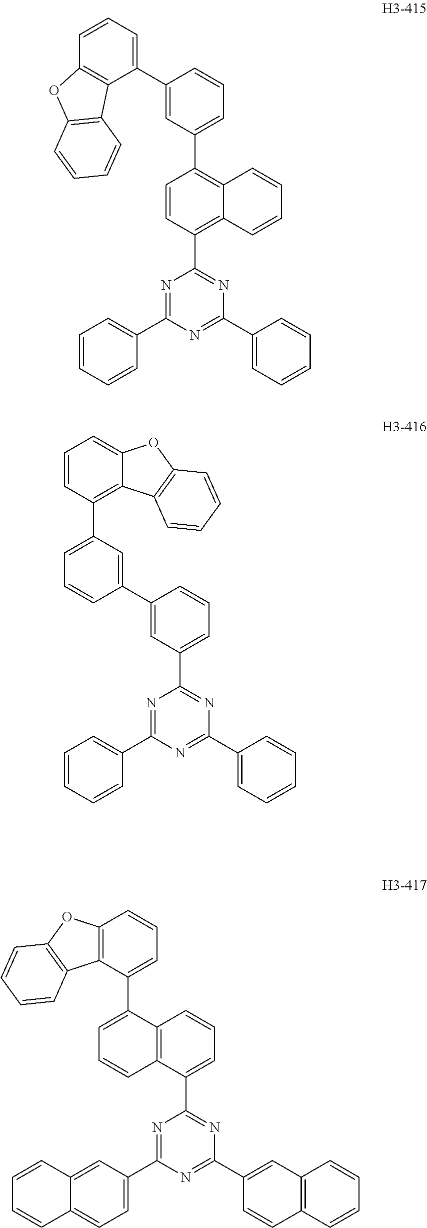 [SMILES: c1ccc(-c2nc(-c3ccccc3)nc(-c3ccc(-c4cccc(-c5cccc6oc7ccccc7c56)c4)c4ccccc34)n2)cc1 ; c1ccc(-c2nc(-c3ccccc3)nc(-c3cccc(-c4cccc(-c5cccc6oc7ccccc7c56)c4)c3)n2)cc1 ; c1ccc2cc(-c3nc(-c4ccc5ccccc5c4)nc(-c4cccc5c(-c6cccc7oc8ccccc8c67)cccc45)n3)ccc2c1]

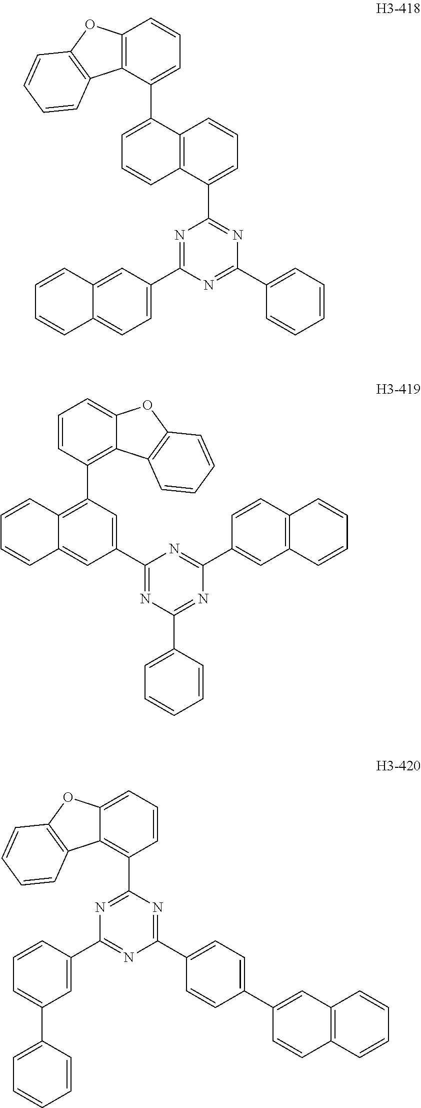 [SMILES: c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1 ; c1ccc(-c2cccc(-c3nc(-c4ccc(-c5ccc6ccccc6c5)cc4)nc(-c4cccc5oc6ccccc6c45)n3)c2)cc1]

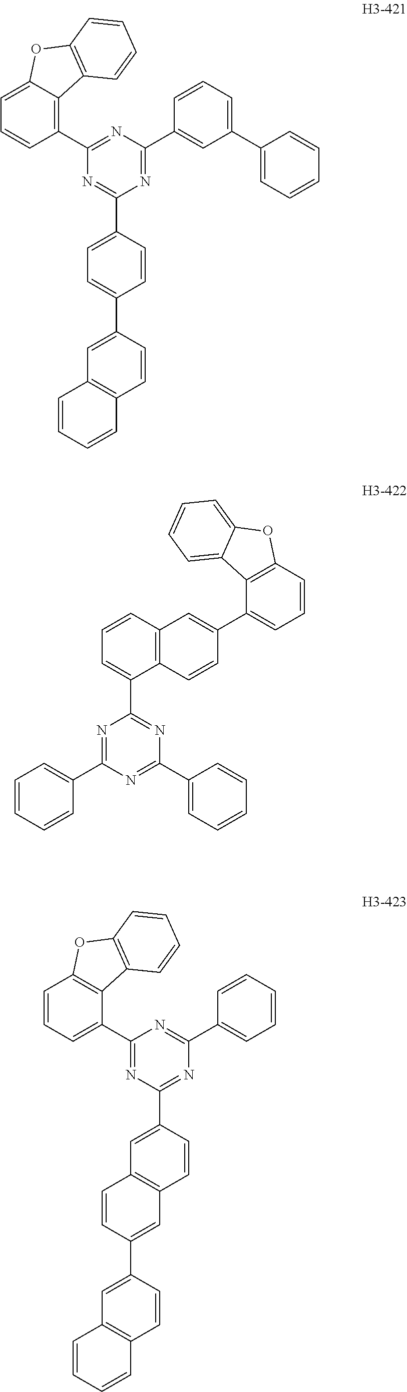 [SMILES: c1ccc(-c2cccc(-c3nc(-c4ccc(-c5ccc6ccccc6c5)cc4)nc(-c4cccc5oc6ccccc6c45)n3)c2)cc1 ; c1ccc(-c2nc(-c3ccccc3)nc(-c3cccc4cc(-c5cccc6oc7ccccc7c56)ccc34)n2)cc1 ; c1ccc(-c2nc(-c3ccc4cc(-c5ccc6ccccc6c5)ccc4c3)nc(-c3cccc4oc5ccccc5c34)n2)cc1]

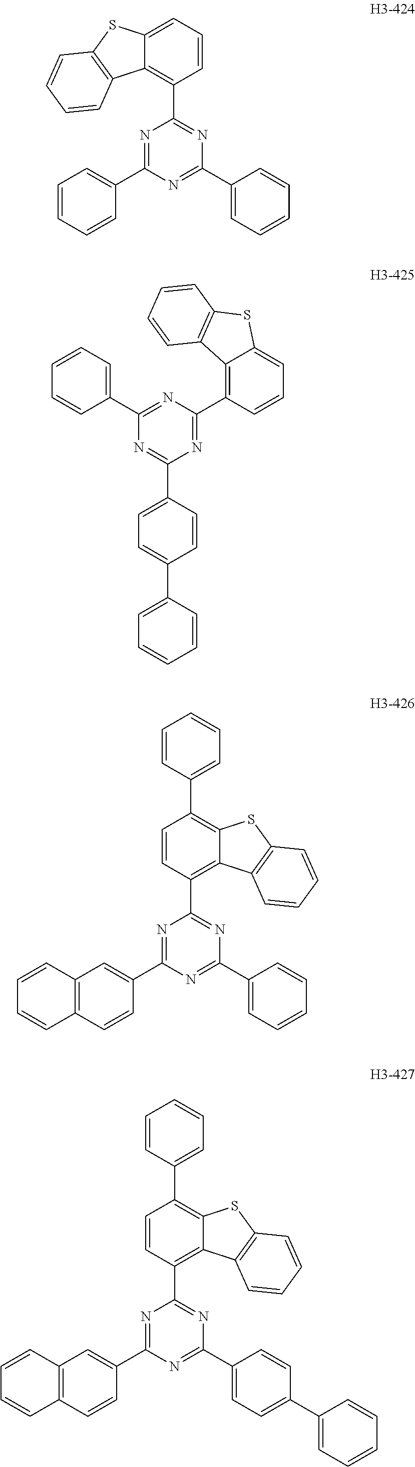 [SMILES: c1ccc(-c2nc(-c3ccccc3)nc(-c3cccc4sc5ccccc5c34)n2)cc1 ; c1ccc(-c2ccc(-c3nc(-c4ccccc4)nc(-c4cccc5sc6ccccc6c45)n3)cc2)cc1 ; c1ccc(-c2nc(-c3ccc4ccccc4c3)nc(-c3ccc(-c4ccccc4)c4sc5ccccc5c34)n2)cc1 ; c1ccc(-c2ccc(-c3nc(-c4ccc5ccccc5c4)nc(-c4ccc(-c5ccccc5)c5sc6ccccc6c45)n3)cc2)cc1]

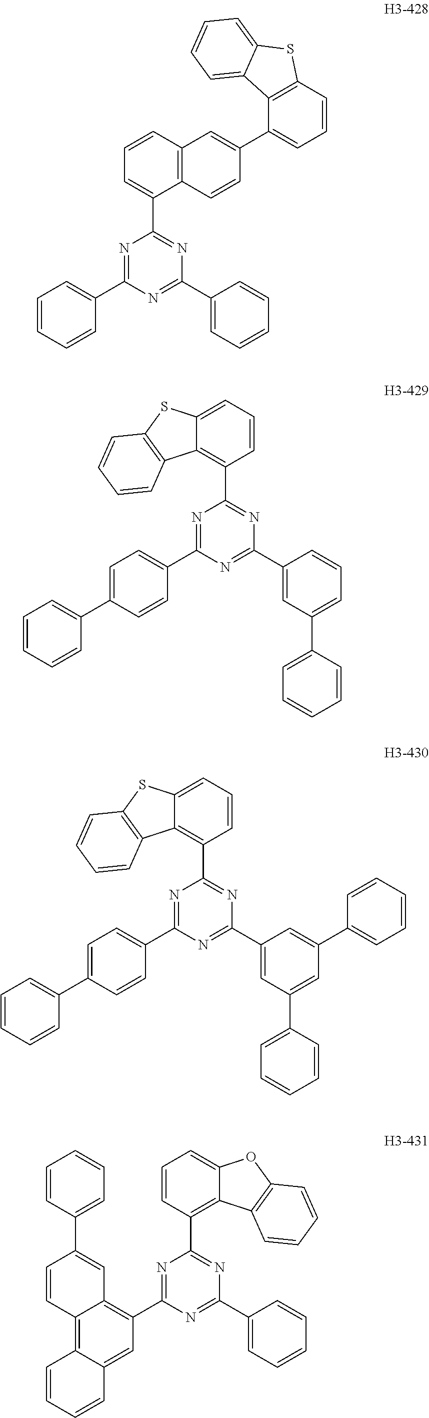 [SMILES: c1ccc(-c2nc(-c3ccccc3)nc(-c3cccc4cc(-c5cccc6sc7ccccc7c56)ccc34)n2)cc1 ; c1ccc(-c2ccc(-c3nc(-c4cccc(-c5ccccc5)c4)nc(-c4cccc5sc6ccccc6c45)n3)cc2)cc1 ; c1ccc(-c2ccc(-c3nc(-c4cc(-c5ccccc5)cc(-c5ccccc5)c4)nc(-c4cccc5sc6ccccc6c45)n3)cc2)cc1 ; c1ccc(-c2ccc3c(c2)c(-c2nc(-c4ccccc4)nc(-c4cccc5oc6ccccc6c45)n2)cc2ccccc23)cc1]

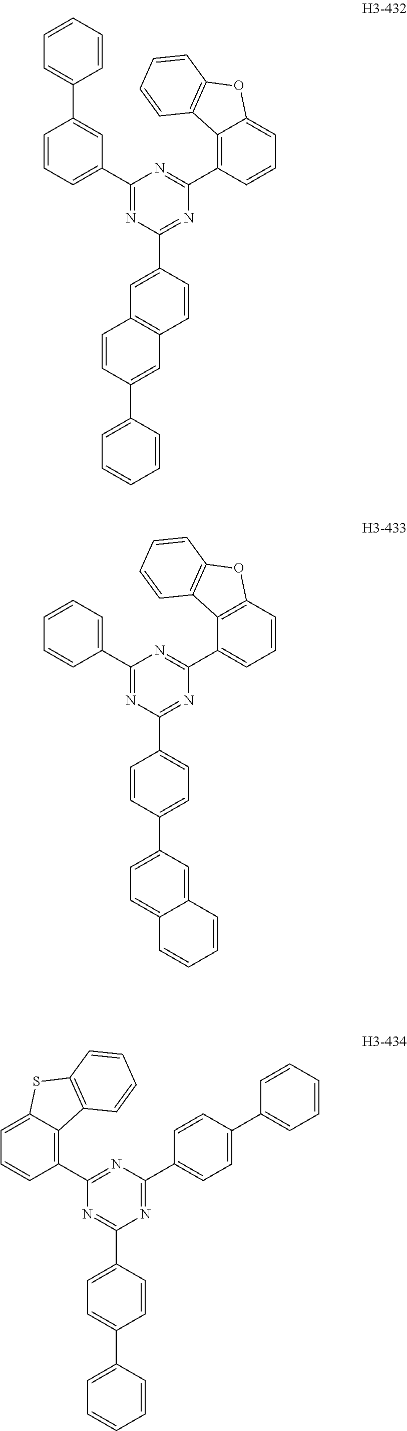 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2nc(-c3ccc(-c4ccc5ccccc5c4)cc3)nc(-c3cccc4oc5ccccc5c34)n2)cc1 ; c1ccc(-c2ccc(-c3nc(-c4ccc(-c5ccccc5)cc4)nc(-c4cccc5sc6ccccc6c45)n3)cc2)cc1]

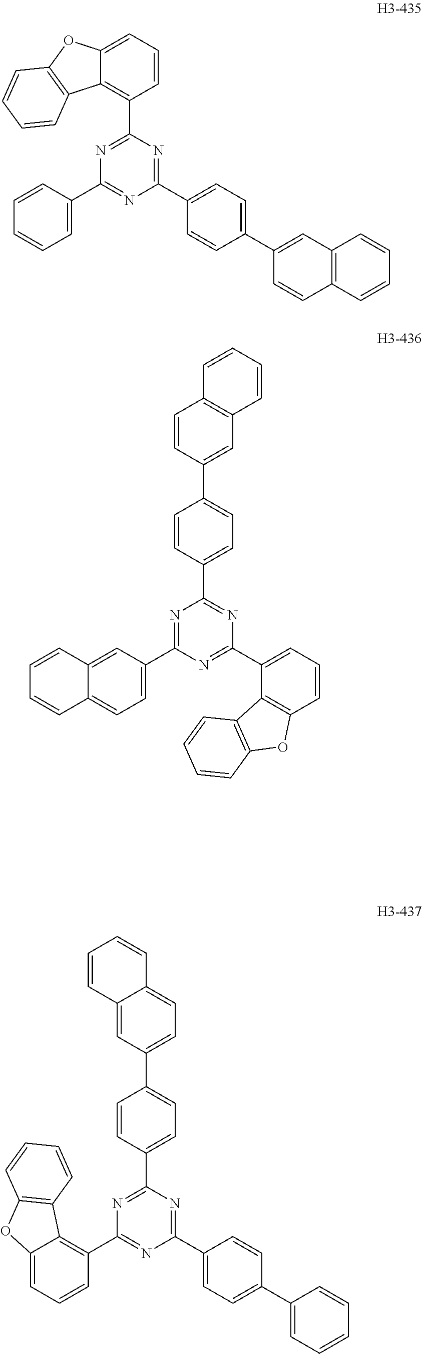 [SMILES: c1ccc(-c2nc(-c3ccc(-c4ccc5ccccc5c4)cc3)nc(-c3cccc4oc5ccccc5c34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccc(-c5ccc6ccccc6c5)cc4)nc(-c4cccc5oc6ccccc6c45)n3)cc2)cc1]

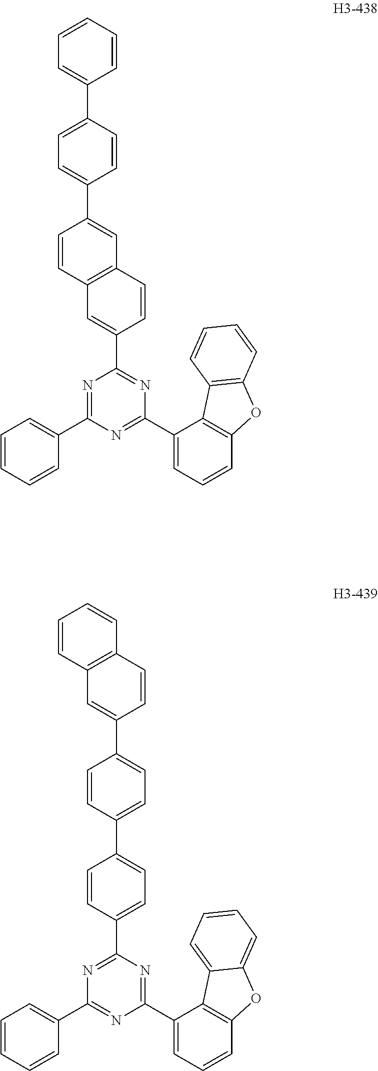 [SMILES: c1ccc(-c2ccc(-c3ccc4cc(-c5nc(-c6ccccc6)nc(-c6cccc7oc8ccccc8c67)n5)ccc4c3)cc2)cc1 ; c1ccc(-c2nc(-c3ccc(-c4ccc(-c5ccc6ccccc6c5)cc4)cc3)nc(-c3cccc4oc5ccccc5c34)n2)cc1]

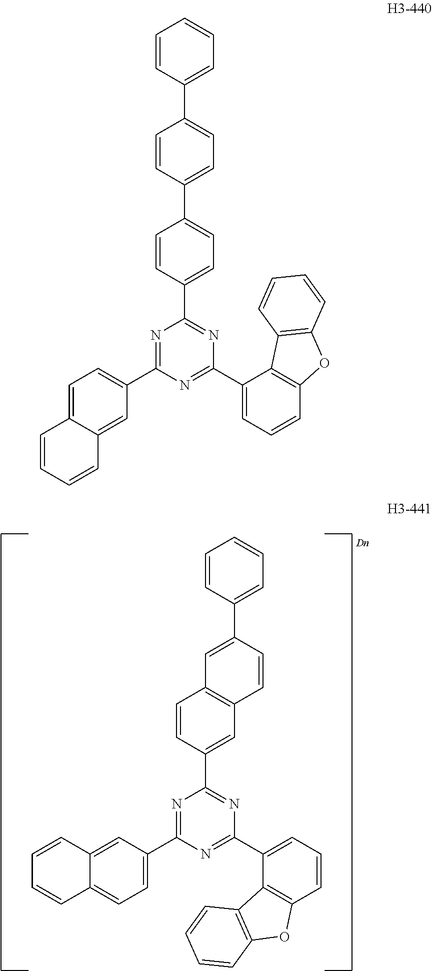

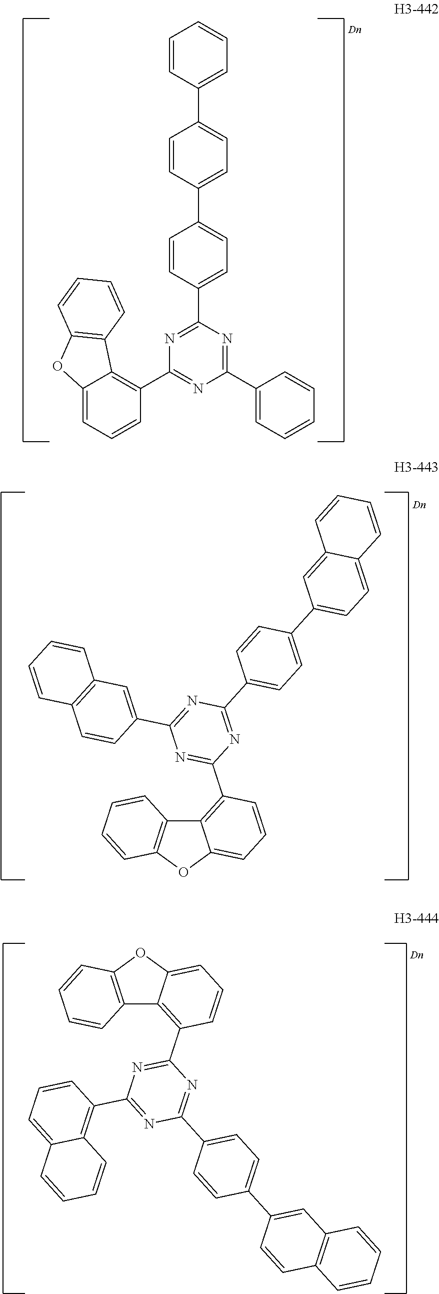

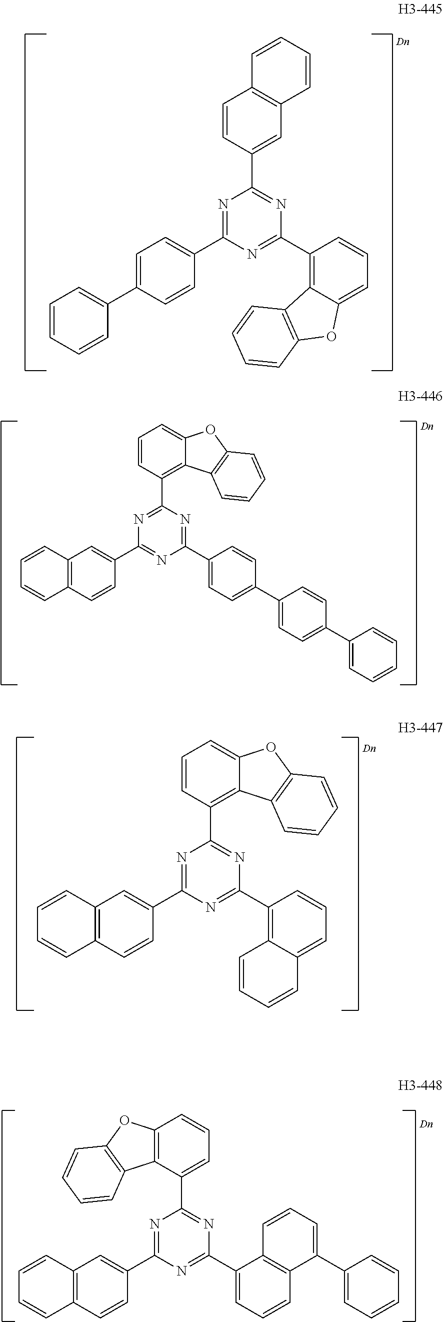

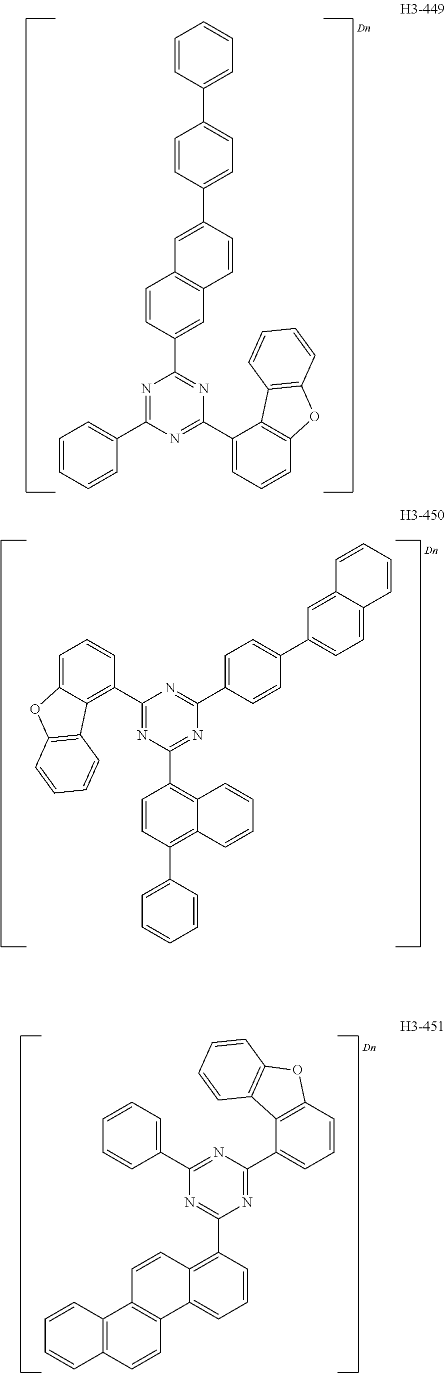

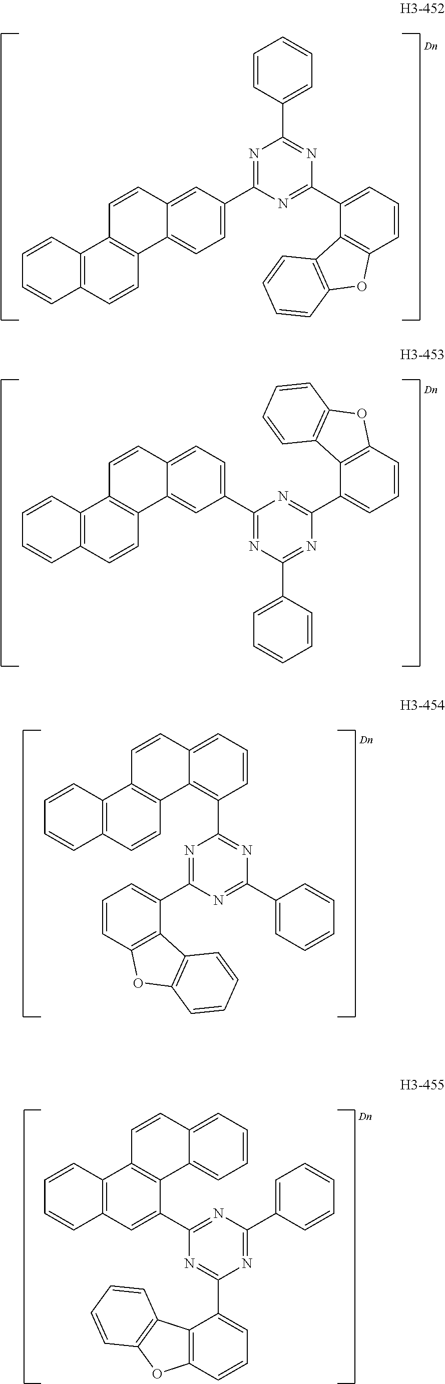

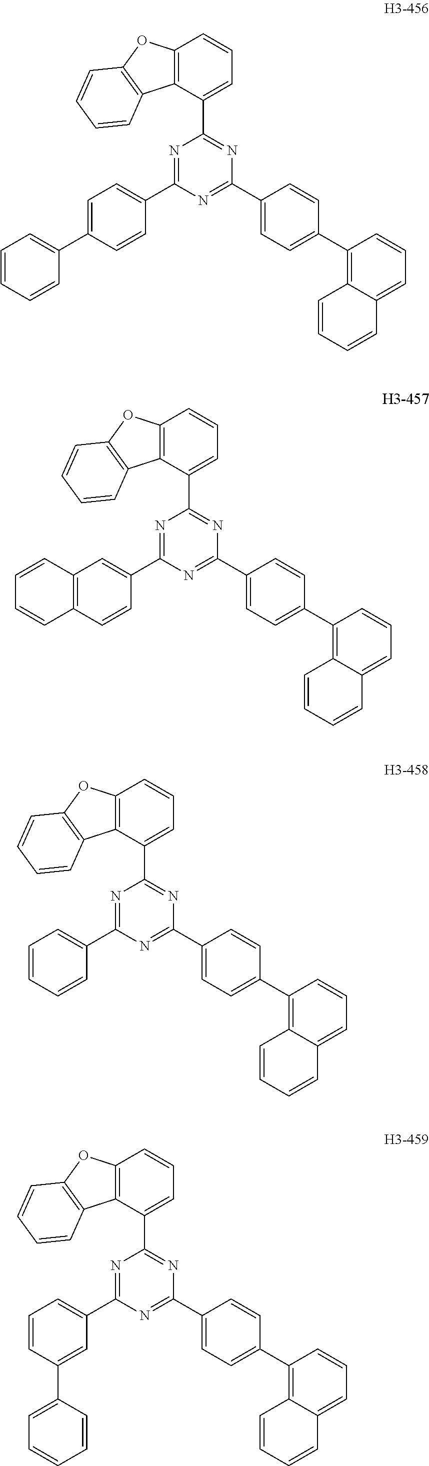 [SMILES: c1ccc(-c2ccc(-c3nc(-c4ccc(-c5cccc6ccccc56)cc4)nc(-c4cccc5oc6ccccc6c45)n3)cc2)cc1 ; c1ccc2cc(-c3nc(-c4ccc(-c5cccc6ccccc56)cc4)nc(-c4cccc5oc6ccccc6c45)n3)ccc2c1 ; c1ccc(-c2nc(-c3ccc(-c4cccc5ccccc45)cc3)nc(-c3cccc4oc5ccccc5c34)n2)cc1 ; c1ccc(-c2cccc(-c3nc(-c4ccc(-c5cccc6ccccc56)cc4)nc(-c4cccc5oc6ccccc6c45)n3)c2)cc1]

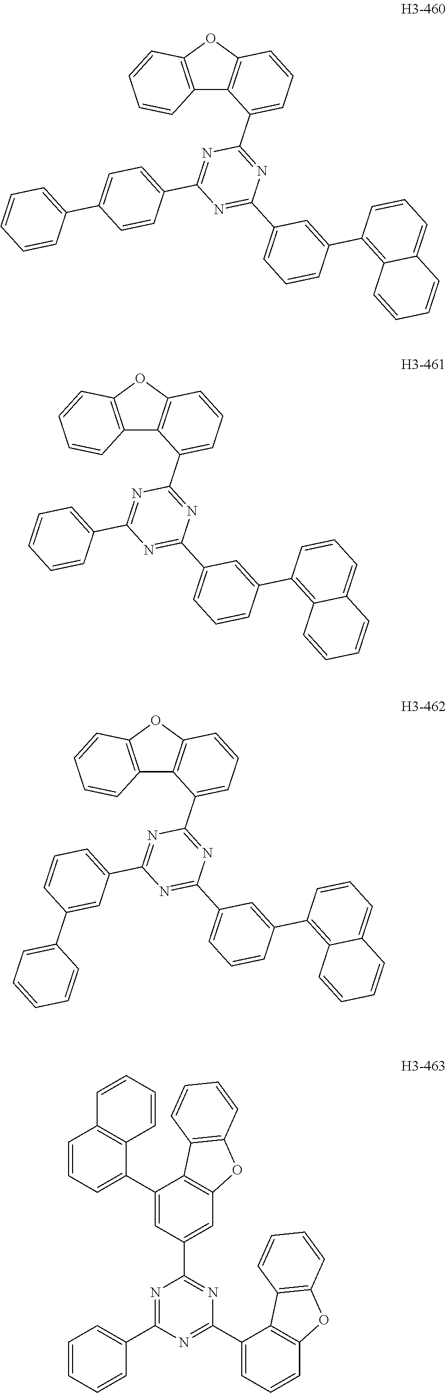 [SMILES: c1ccc(-c2ccc(-c3nc(-c4cccc(-c5cccc6ccccc56)c4)nc(-c4cccc5oc6ccccc6c45)n3)cc2)cc1 ; c1ccc(-c2nc(-c3cccc(-c4cccc5ccccc45)c3)nc(-c3cccc4oc5ccccc5c34)n2)cc1 ; c1ccc(-c2cccc(-c3nc(-c4cccc(-c5cccc6ccccc56)c4)nc(-c4cccc5oc6ccccc6c45)n3)c2)cc1 ; c1ccc(-c2nc(-c3cc(-c4cccc5ccccc45)c4c(c3)oc3ccccc34)nc(-c3cccc4oc5ccccc5c34)n2)cc1]

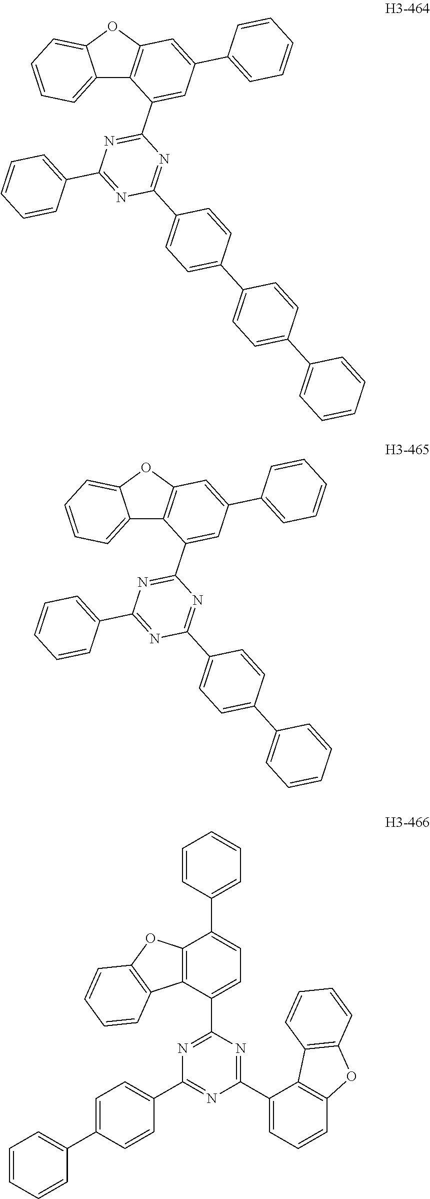

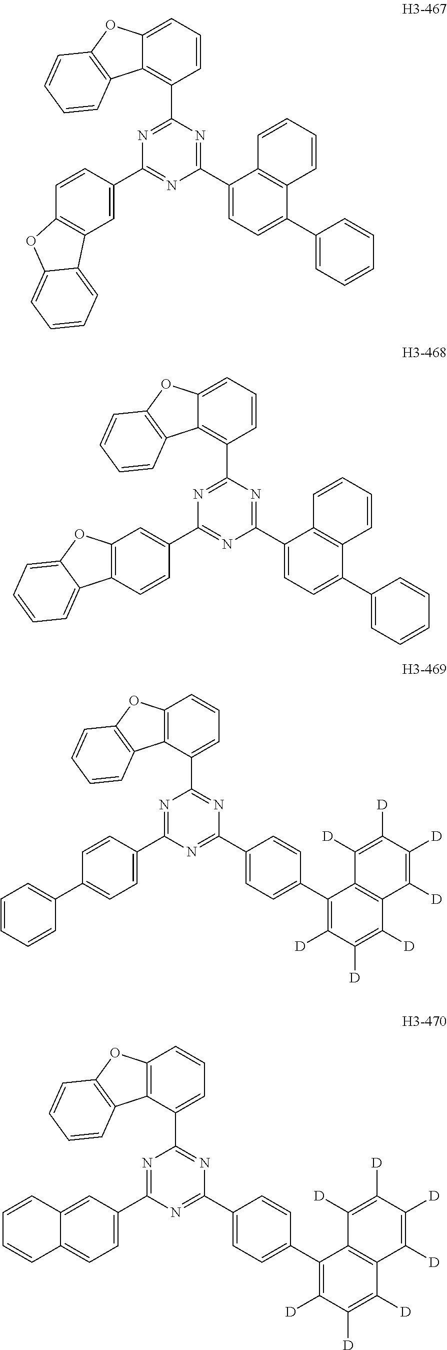 [SMILES: c1ccc(-c2ccc(-c3nc(-c4ccc5oc6ccccc6c5c4)nc(-c4cccc5oc6ccccc6c45)n3)c3ccccc23)cc1 ; c1ccc(-c2ccc(-c3nc(-c4ccc5c(c4)oc4ccccc45)nc(-c4cccc5oc6ccccc6c45)n3)c3ccccc23)cc1 ; [2H]c1c([2H])c([2H])c2c(-c3ccc(-c4nc(-c5ccc(-c6ccccc6)cc5)nc(-c5cccc6oc7ccccc7c56)n4)cc3)c([2H])c([2H])c([2H])c2c1[2H] ; [2H]c1c([2H])c([2H])c2c(-c3ccc(-c4nc(-c5ccc6ccccc6c5)nc(-c5cccc6oc7ccccc7c56)n4)cc3)c([2H])c([2H])c([2H])c2c1[2H]]

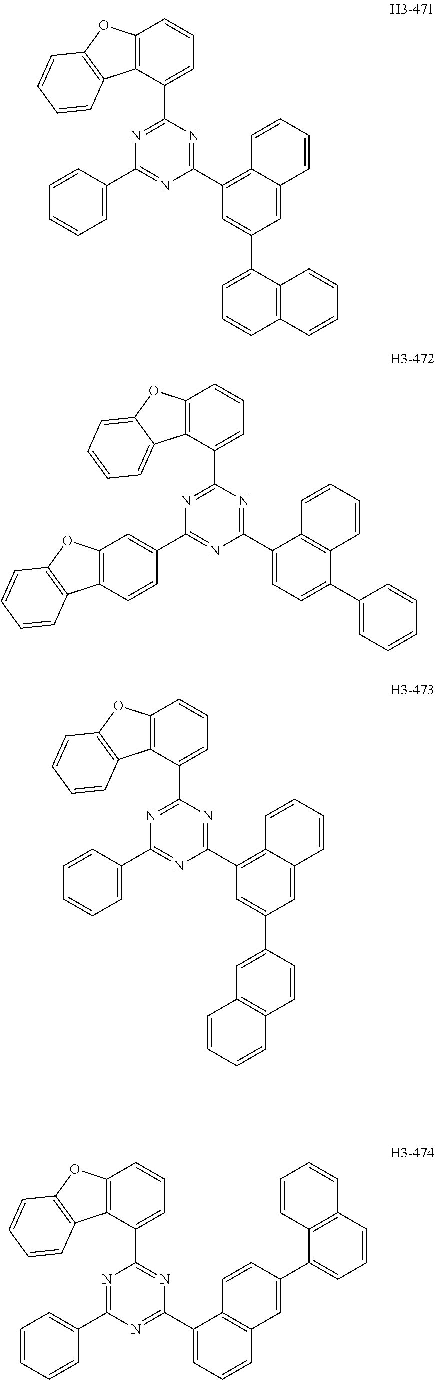 [SMILES: c1ccc(-c2nc(-c3cc(-c4cccc5ccccc45)cc4ccccc34)nc(-c3cccc4oc5ccccc5c34)n2)cc1 ; c1ccc(-c2ccc(-c3nc(-c4ccc5c(c4)oc4ccccc45)nc(-c4cccc5oc6ccccc6c45)n3)c3ccccc23)cc1 ; c1ccc(-c2nc(-c3cc(-c4ccc5ccccc5c4)cc4ccccc34)nc(-c3cccc4oc5ccccc5c34)n2)cc1 ; c1ccc(-c2nc(-c3cccc4cc(-c5cccc6ccccc56)ccc34)nc(-c3cccc4oc5ccccc5c34)n2)cc1]

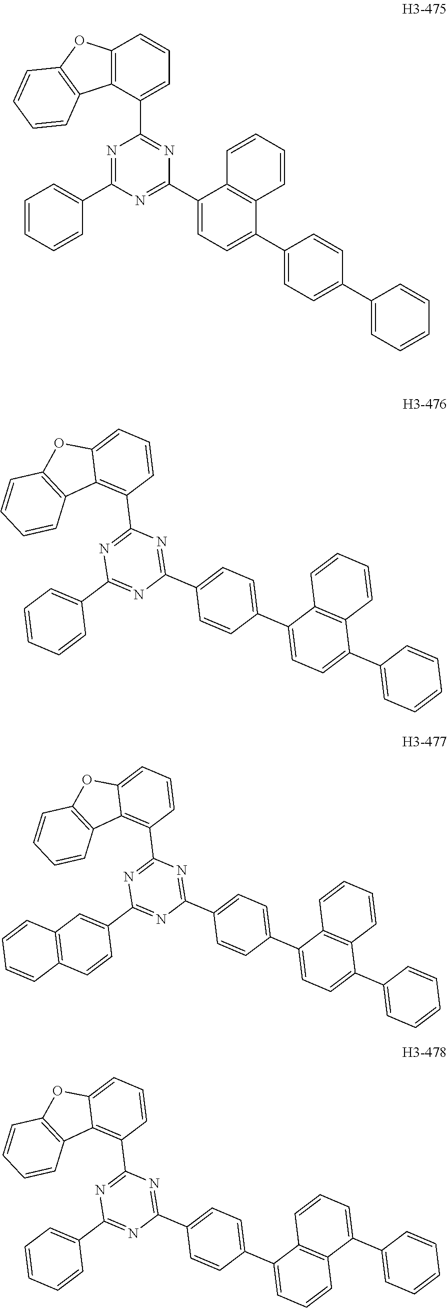 [SMILES: c1ccc(-c2ccc(-c3ccc(-c4nc(-c5ccccc5)nc(-c5cccc6oc7ccccc7c56)n4)c4ccccc34)cc2)cc1 ; c1ccc(-c2nc(-c3ccc(-c4ccc(-c5ccccc5)c5ccccc45)cc3)nc(-c3cccc4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)c3ccccc23)cc1 ; c1ccc(-c2nc(-c3ccc(-c4cccc5c(-c6ccccc6)cccc45)cc3)nc(-c3cccc4oc5ccccc5c34)n2)cc1]

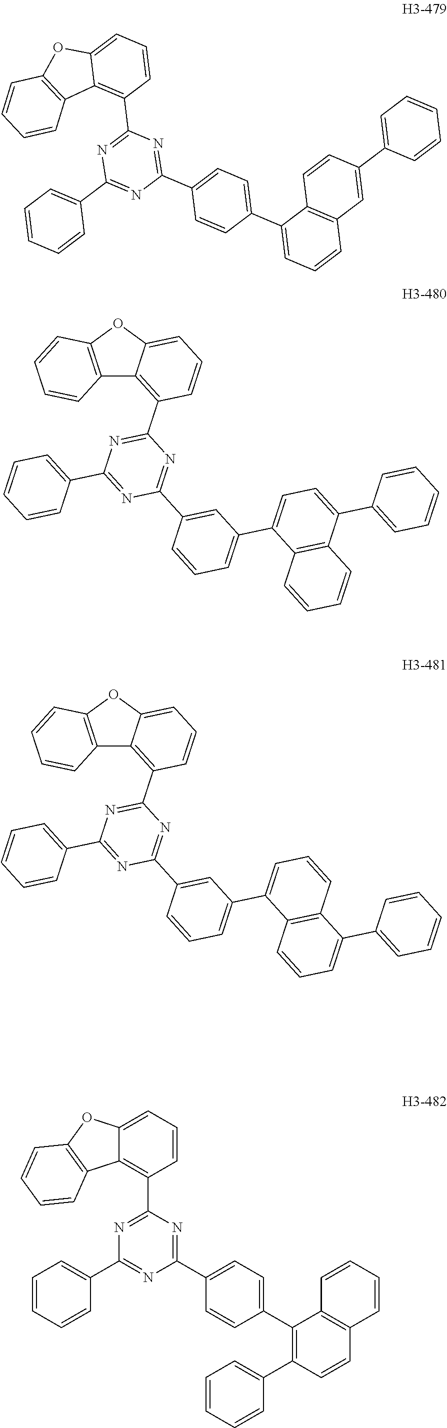 [SMILES: c1ccc(-c2ccc3c(-c4ccc(-c5nc(-c6ccccc6)nc(-c6cccc7oc8ccccc8c67)n5)cc4)cccc3c2)cc1 ; c1ccc(-c2nc(-c3cccc(-c4ccc(-c5ccccc5)c5ccccc45)c3)nc(-c3cccc4oc5ccccc5c34)n2)cc1 ; c1ccc(-c2nc(-c3cccc(-c4cccc5c(-c6ccccc6)cccc45)c3)nc(-c3cccc4oc5ccccc5c34)n2)cc1 ; c1ccc(-c2nc(-c3ccc(-c4c(-c5ccccc5)ccc5ccccc45)cc3)nc(-c3cccc4oc5ccccc5c34)n2)cc1]

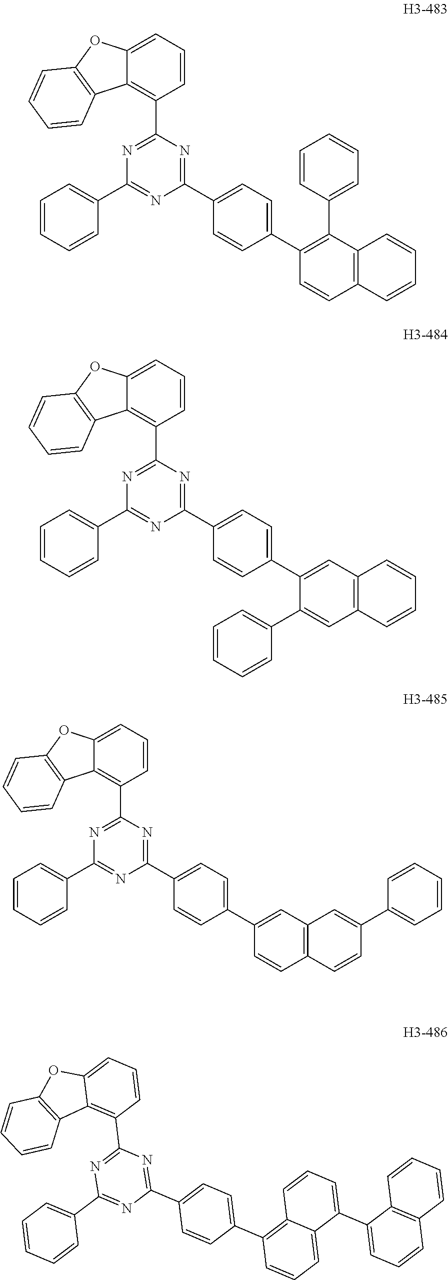 [SMILES: c1ccc(-c2nc(-c3ccc(-c4ccc5ccccc5c4-c4ccccc4)cc3)nc(-c3cccc4oc5ccccc5c34)n2)cc1 ; c1ccc(-c2nc(-c3ccc(-c4cc5ccccc5cc4-c4ccccc4)cc3)nc(-c3cccc4oc5ccccc5c34)n2)cc1 ; c1ccc(-c2ccc3ccc(-c4ccc(-c5nc(-c6ccccc6)nc(-c6cccc7oc8ccccc8c67)n5)cc4)cc3c2)cc1 ; c1ccc(-c2nc(-c3ccc(-c4cccc5c(-c6cccc7ccccc67)cccc45)cc3)nc(-c3cccc4oc5ccccc5c34)n2)cc1]

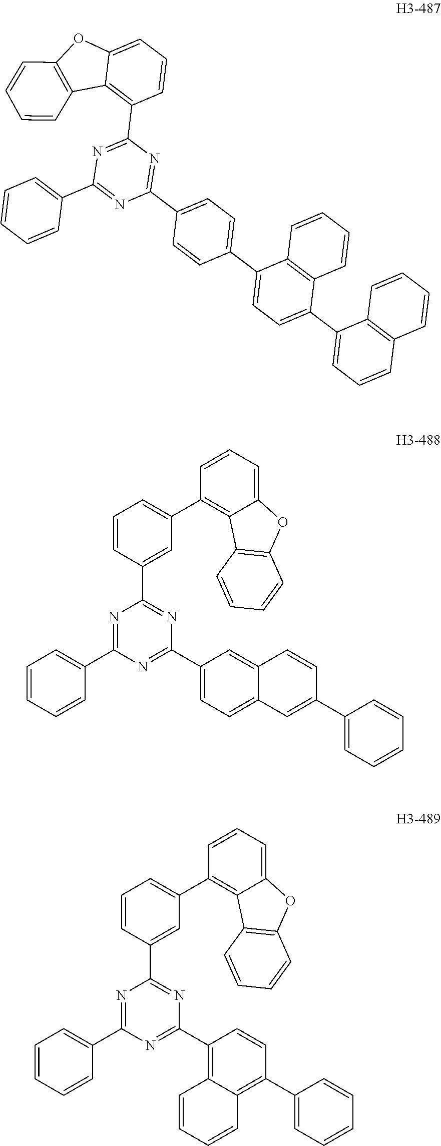 [SMILES: c1ccc(-c2nc(-c3ccc(-c4ccc(-c5cccc6ccccc56)c5ccccc45)cc3)nc(-c3cccc4oc5ccccc5c34)n2)cc1 ; c1ccc(-c2ccc3cc(-c4nc(-c5ccccc5)nc(-c5cccc(-c6cccc7oc8ccccc8c67)c5)n4)ccc3c2)cc1 ; c1ccc(-c2nc(-c3cccc(-c4cccc5oc6ccccc6c45)c3)nc(-c3ccc(-c4ccccc4)c4ccccc34)n2)cc1]

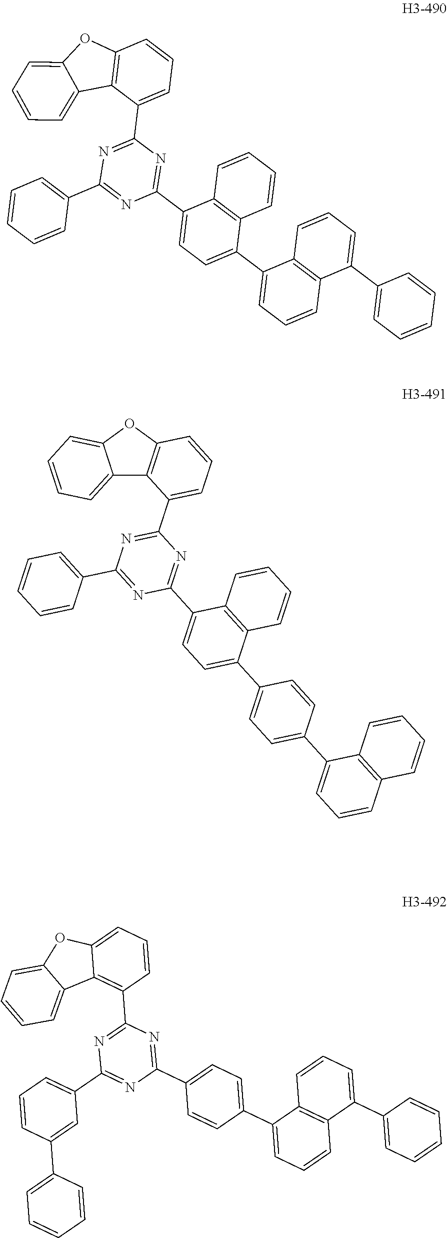 [SMILES: c1ccc(-c2nc(-c3ccc(-c4cccc5c(-c6ccccc6)cccc45)c4ccccc34)nc(-c3cccc4oc5ccccc5c34)n2)cc1 ; c1ccc(-c2nc(-c3ccc(-c4ccc(-c5cccc6ccccc56)cc4)c4ccccc34)nc(-c3cccc4oc5ccccc5c34)n2)cc1 ; c1ccc(-c2cccc(-c3nc(-c4ccc(-c5cccc6c(-c7ccccc7)cccc56)cc4)nc(-c4cccc5oc6ccccc6c45)n3)c2)cc1]

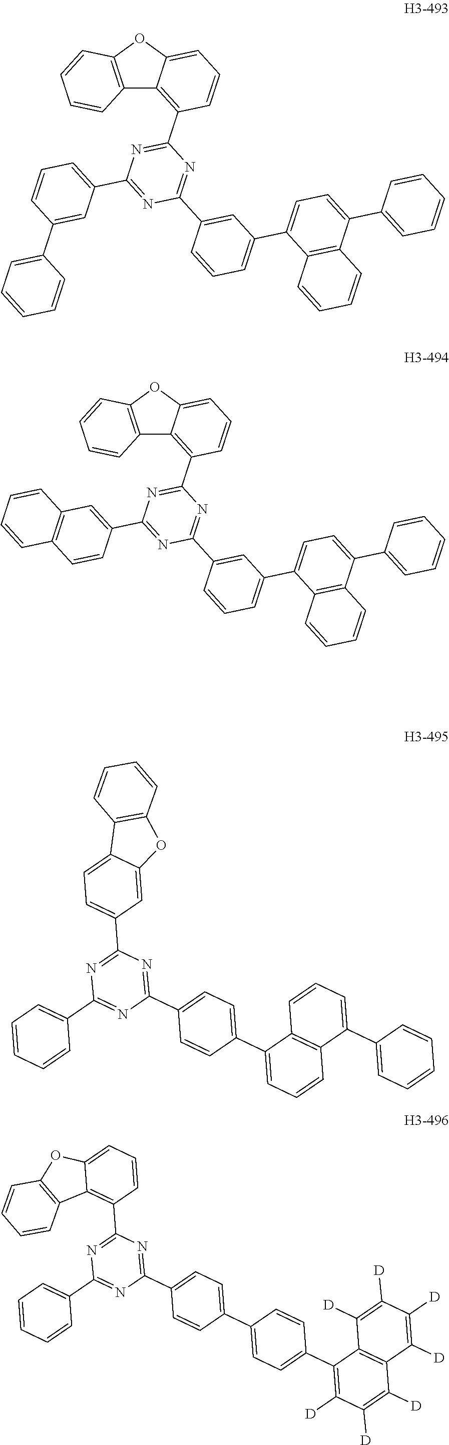 [SMILES: c1ccc(-c2cccc(-c3nc(-c4cccc(-c5ccc(-c6ccccc6)c6ccccc56)c4)nc(-c4cccc5oc6ccccc6c45)n3)c2)cc1 ; c1ccc(-c2ccc(-c3cccc(-c4nc(-c5ccc6ccccc6c5)nc(-c5cccc6oc7ccccc7c56)n4)c3)c3ccccc23)cc1 ; c1ccc(-c2nc(-c3ccc(-c4cccc5c(-c6ccccc6)cccc45)cc3)nc(-c3ccc4c(c3)oc3ccccc34)n2)cc1 ; [2H]c1c([2H])c([2H])c2c(-c3ccc(-c4ccc(-c5nc(-c6ccccc6)nc(-c6cccc7oc8ccccc8c67)n5)cc4)cc3)c([2H])c([2H])c([2H])c2c1[2H]]

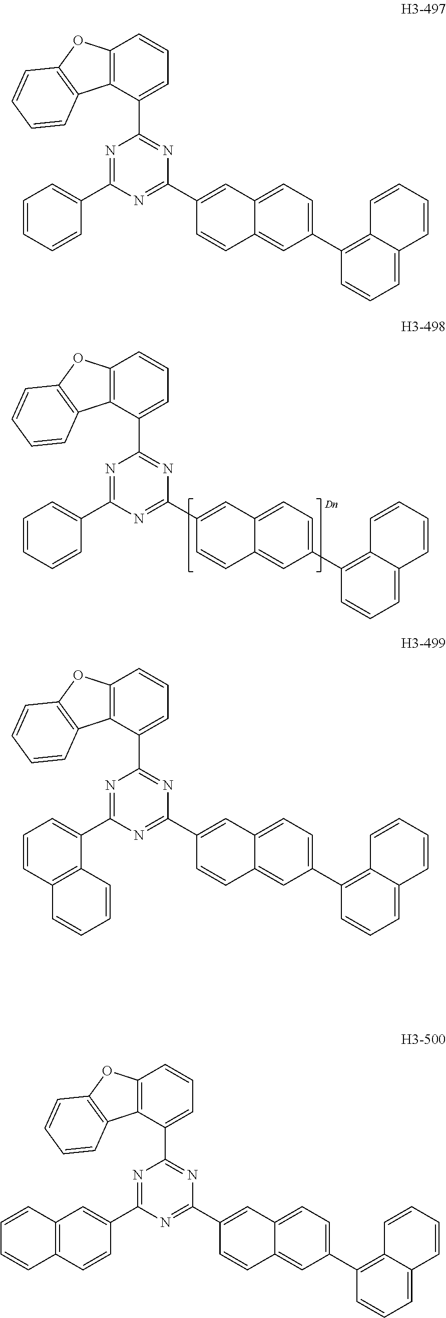

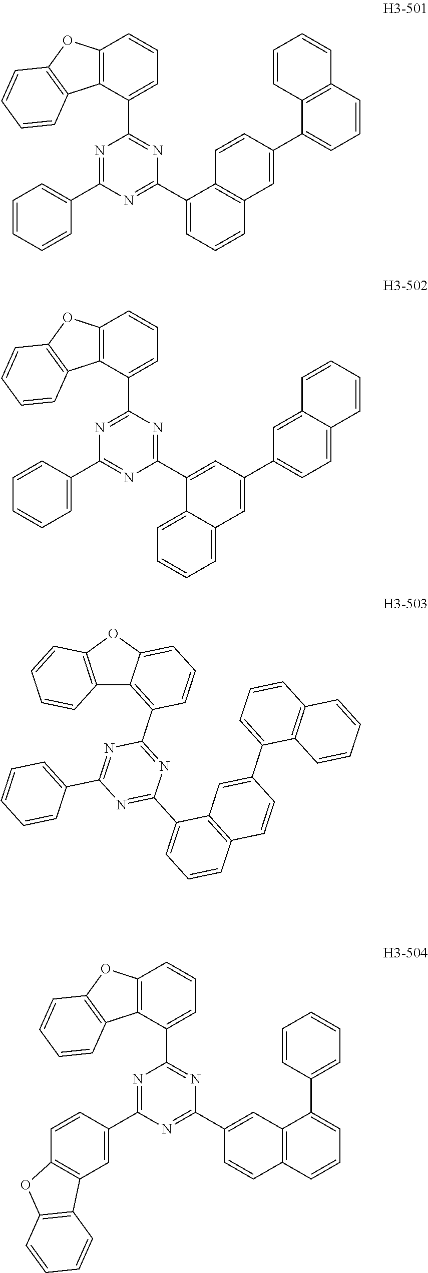 [SMILES: c1ccc(-c2nc(-c3cccc4cc(-c5cccc6ccccc56)ccc34)nc(-c3cccc4oc5ccccc5c34)n2)cc1 ; c1ccc(-c2nc(-c3cc(-c4ccc5ccccc5c4)cc4ccccc34)nc(-c3cccc4oc5ccccc5c34)n2)cc1 ; c1ccc(-c2nc(-c3cccc4ccc(-c5cccc6ccccc56)cc34)nc(-c3cccc4oc5ccccc5c34)n2)cc1 ; c1ccc(-c2cccc3ccc(-c4nc(-c5ccc6oc7ccccc7c6c5)nc(-c5cccc6oc7ccccc7c56)n4)cc23)cc1]

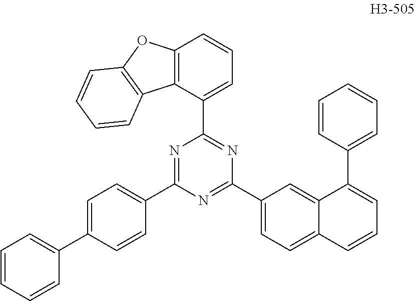 [SMILES: c1ccc(-c2ccc(-c3nc(-c4ccc5cccc(-c6ccccc6)c5c4)nc(-c4cccc5oc6ccccc6c45)n3)cc2)cc1]

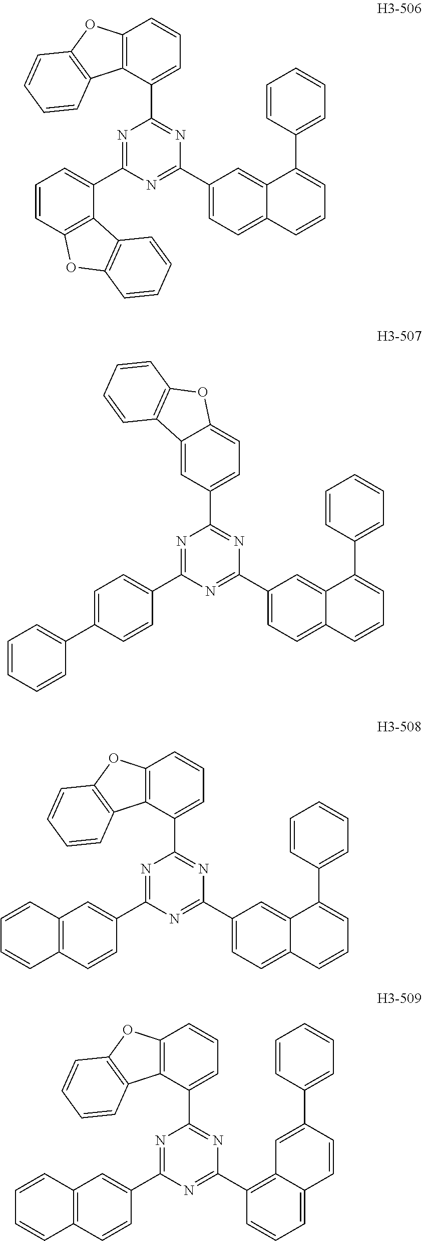 [SMILES: c1ccc(-c2cccc3ccc(-c4nc(-c5cccc6oc7ccccc7c56)nc(-c5cccc6oc7ccccc7c56)n4)cc23)cc1 ; c1ccc(-c2ccc(-c3nc(-c4ccc5cccc(-c6ccccc6)c5c4)nc(-c4ccc5oc6ccccc6c5c4)n3)cc2)cc1 ; c1ccc(-c2cccc3ccc(-c4nc(-c5ccc6ccccc6c5)nc(-c5cccc6oc7ccccc7c56)n4)cc23)cc1 ; c1ccc(-c2ccc3cccc(-c4nc(-c5ccc6ccccc6c5)nc(-c5cccc6oc7ccccc7c56)n4)c3c2)cc1]

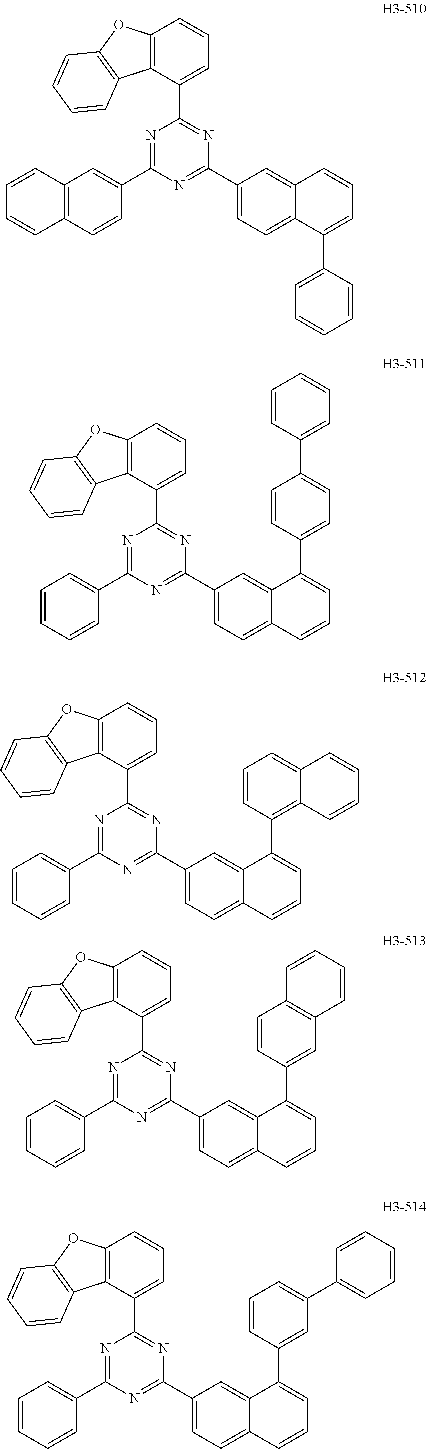

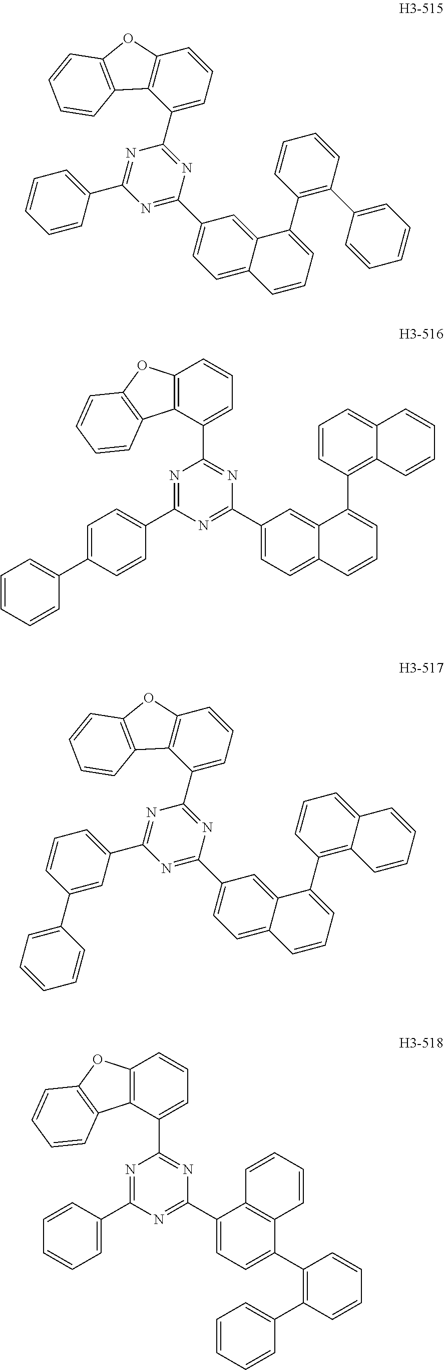 [SMILES: c1ccc(-c2nc(-c3ccc4cccc(-c5ccccc5-c5ccccc5)c4c3)nc(-c3cccc4oc5ccccc5c34)n2)cc1 ; c1ccc(-c2ccc(-c3nc(-c4ccc5cccc(-c6cccc7ccccc67)c5c4)nc(-c4cccc5oc6ccccc6c45)n3)cc2)cc1 ; c1ccc(-c2cccc(-c3nc(-c4ccc5cccc(-c6cccc7ccccc67)c5c4)nc(-c4cccc5oc6ccccc6c45)n3)c2)cc1 ; c1ccc(-c2nc(-c3ccc(-c4ccccc4-c4ccccc4)c4ccccc34)nc(-c3cccc4oc5ccccc5c34)n2)cc1]

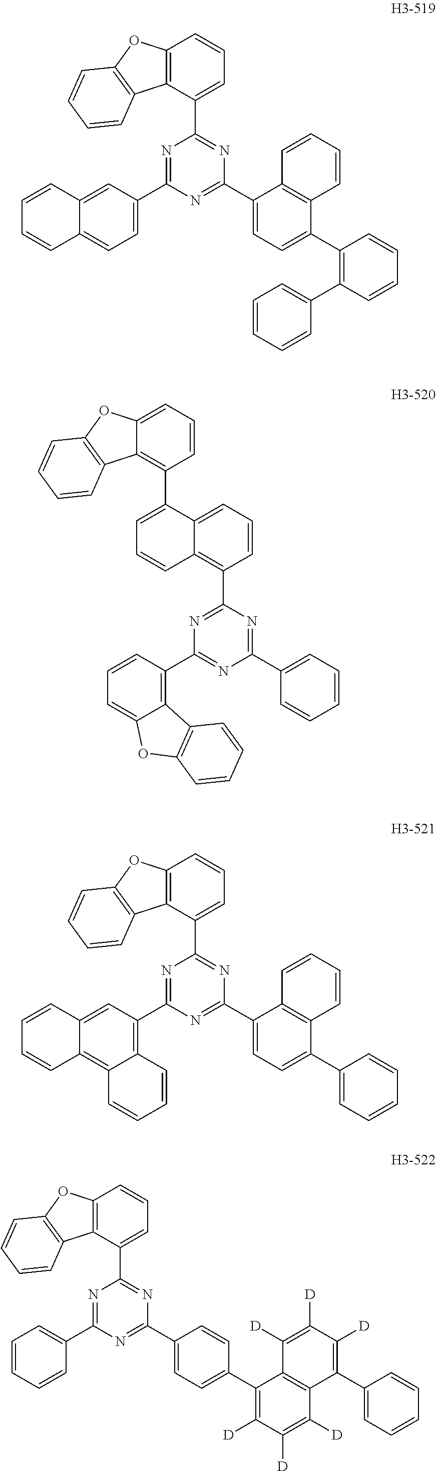 [SMILES: c1ccc(-c2ccccc2-c2ccc(-c3nc(-c4ccc5ccccc5c4)nc(-c4cccc5oc6ccccc6c45)n3)c3ccccc23)cc1 ; c1ccc(-c2nc(-c3cccc4c(-c5cccc6oc7ccccc7c56)cccc34)nc(-c3cccc4oc5ccccc5c34)n2)cc1 ; c1ccc(-c2ccc(-c3nc(-c4cc5ccccc5c5ccccc45)nc(-c4cccc5oc6ccccc6c45)n3)c3ccccc23)cc1 ; [2H]c1c([2H])c(-c2ccc(-c3nc(-c4ccccc4)nc(-c4cccc5oc6ccccc6c45)n3)cc2)c2c([2H])c([2H])c([2H])c(-c3ccccc3)c2c1[2H]]

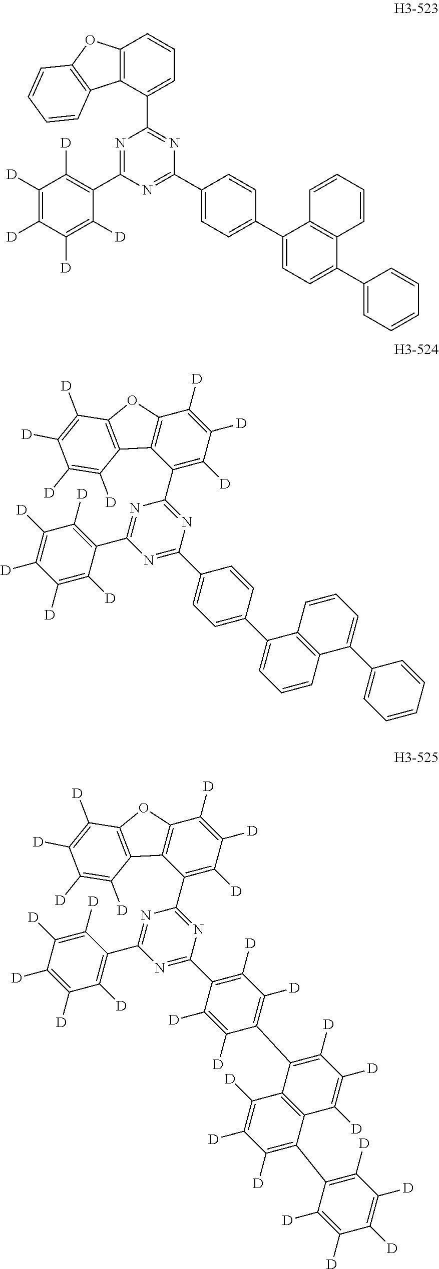 [SMILES: [2H]c1c([2H])c([2H])c(-c2nc(-c3ccc(-c4ccc(-c5ccccc5)c5ccccc45)cc3)nc(-c3cccc4oc5ccccc5c34)n2)c([2H])c1[2H] ; [2H]c1c([2H])c([2H])c(-c2nc(-c3ccc(-c4cccc5c(-c6ccccc6)cccc45)cc3)nc(-c3c([2H])c([2H])c([2H])c4oc5c([2H])c([2H])c([2H])c([2H])c5c34)n2)c([2H])c1[2H] ; [2H]c1c([2H])c([2H])c(-c2nc(-c3c([2H])c([2H])c(-c4c([2H])c([2H])c([2H])c5c(-c6c([2H])c([2H])c([2H])c([2H])c6[2H])c([2H])c([2H])c([2H])c45)c([2H])c3[2H])nc(-c3c([2H])c([2H])c([2H])c4oc5c([2H])c([2H])c([2H])c([2H])c5c34)n2)c([2H])c1[2H]]

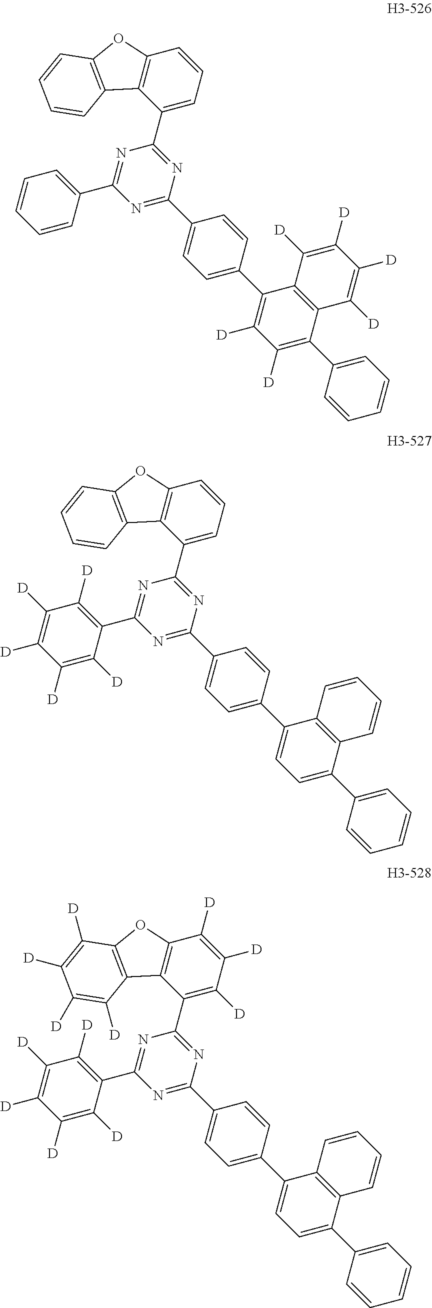 [SMILES: [2H]c1c([2H])c([2H])c2c(-c3ccc(-c4nc(-c5ccccc5)nc(-c5cccc6oc7ccccc7c56)n4)cc3)c([2H])c([2H])c(-c3ccccc3)c2c1[2H] ; [2H]c1c([2H])c([2H])c(-c2nc(-c3ccc(-c4ccc(-c5ccccc5)c5ccccc45)cc3)nc(-c3cccc4oc5ccccc5c34)n2)c([2H])c1[2H] ; [2H]c1c([2H])c([2H])c(-c2nc(-c3ccc(-c4ccc(-c5ccccc5)c5ccccc45)cc3)nc(-c3c([2H])c([2H])c([2H])c4oc5c([2H])c([2H])c([2H])c([2H])c5c34)n2)c([2H])c1[2H]]

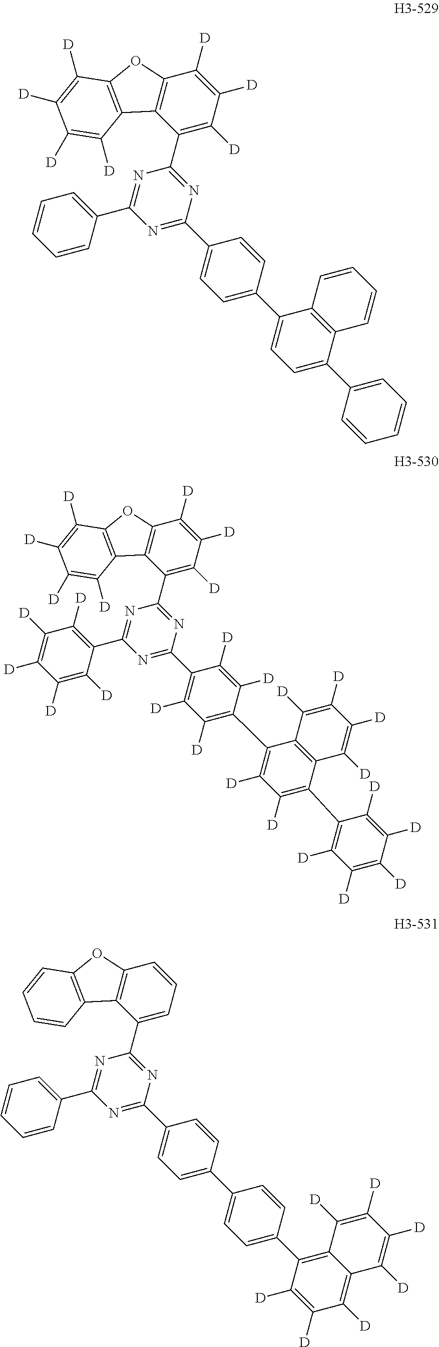 [SMILES: [2H]c1c([2H])c([2H])c2c(oc3c([2H])c([2H])c([2H])c(-c4nc(-c5ccccc5)nc(-c5ccc(-c6ccc(-c7ccccc7)c7ccccc67)cc5)n4)c32)c1[2H] ; [2H]c1c([2H])c([2H])c(-c2nc(-c3c([2H])c([2H])c(-c4c([2H])c([2H])c(-c5c([2H])c([2H])c([2H])c([2H])c5[2H])c5c([2H])c([2H])c([2H])c([2H])c45)c([2H])c3[2H])nc(-c3c([2H])c([2H])c([2H])c4oc5c([2H])c([2H])c([2H])c([2H])c5c34)n2)c([2H])c1[2H] ; [2H]c1c([2H])c([2H])c2c(-c3ccc(-c4ccc(-c5nc(-c6ccccc6)nc(-c6cccc7oc8ccccc8c67)n5)cc4)cc3)c([2H])c([2H])c([2H])c2c1[2H]]

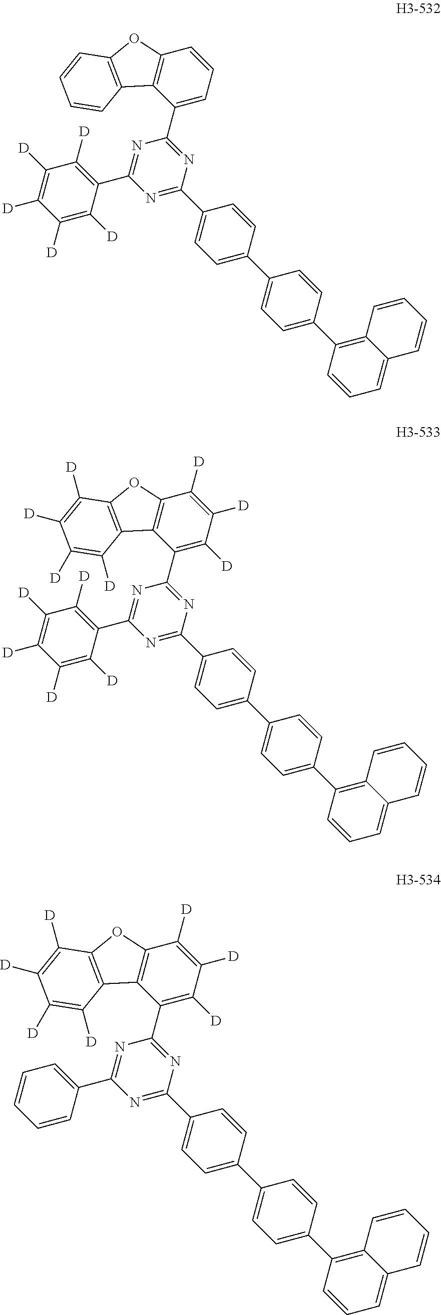 [SMILES: [2H]c1c([2H])c([2H])c(-c2nc(-c3ccc(-c4ccc(-c5cccc6ccccc56)cc4)cc3)nc(-c3cccc4oc5ccccc5c34)n2)c([2H])c1[2H] ; [2H]c1c([2H])c([2H])c(-c2nc(-c3ccc(-c4ccc(-c5cccc6ccccc56)cc4)cc3)nc(-c3c([2H])c([2H])c([2H])c4oc5c([2H])c([2H])c([2H])c([2H])c5c34)n2)c([2H])c1[2H] ; [2H]c1c([2H])c([2H])c2c(oc3c([2H])c([2H])c([2H])c(-c4nc(-c5ccccc5)nc(-c5ccc(-c6ccc(-c7cccc8ccccc78)cc6)cc5)n4)c32)c1[2H]]

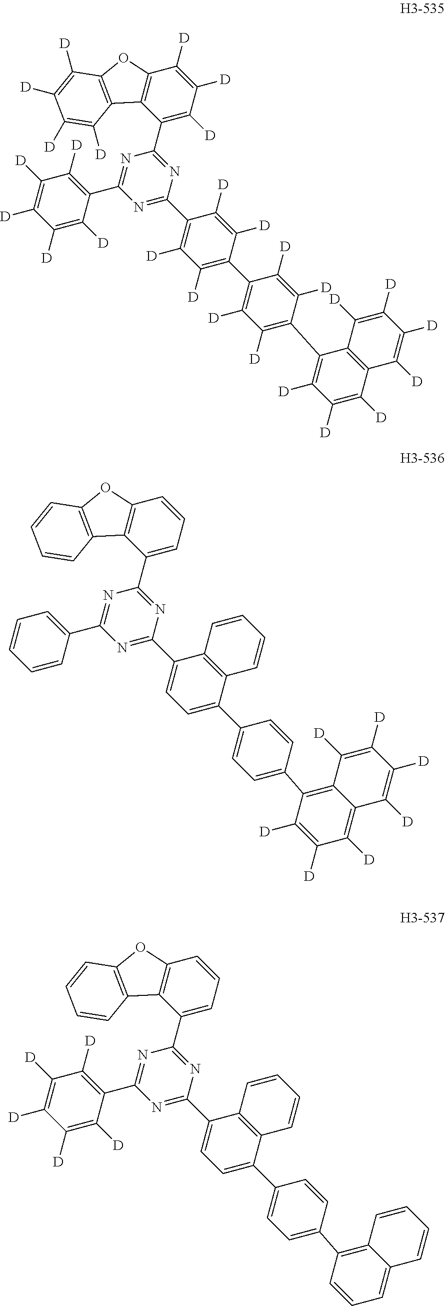 [SMILES: [2H]c1c([2H])c([2H])c(-c2nc(-c3c([2H])c([2H])c(-c4c([2H])c([2H])c(-c5c([2H])c([2H])c([2H])c6c([2H])c([2H])c([2H])c([2H])c56)c([2H])c4[2H])c([2H])c3[2H])nc(-c3c([2H])c([2H])c([2H])c4oc5c([2H])c([2H])c([2H])c([2H])c5c34)n2)c([2H])c1[2H] ; [2H]c1c([2H])c([2H])c2c(-c3ccc(-c4ccc(-c5nc(-c6ccccc6)nc(-c6cccc7oc8ccccc8c67)n5)c5ccccc45)cc3)c([2H])c([2H])c([2H])c2c1[2H] ; [2H]c1c([2H])c([2H])c(-c2nc(-c3ccc(-c4ccc(-c5cccc6ccccc56)cc4)c4ccccc34)nc(-c3cccc4oc5ccccc5c34)n2)c([2H])c1[2H]]

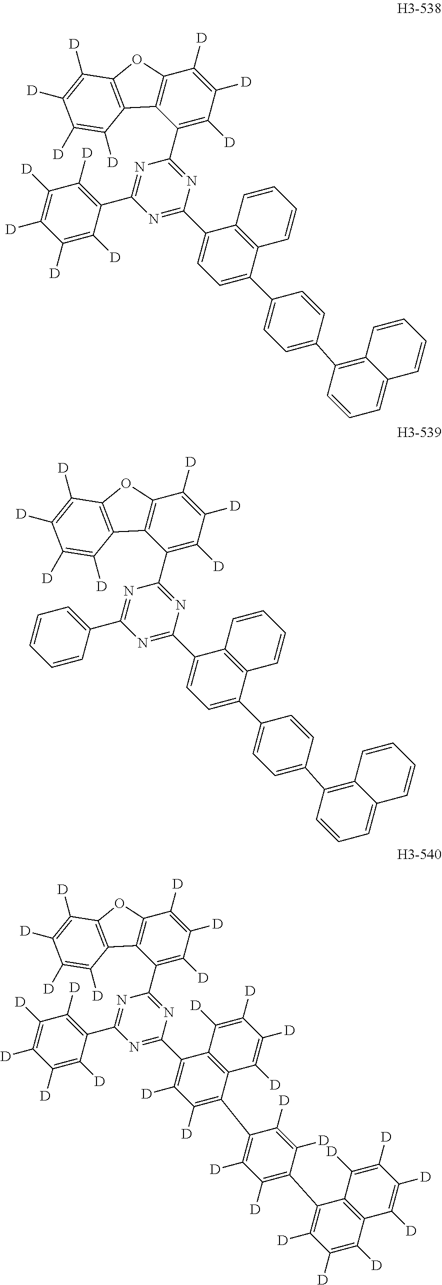

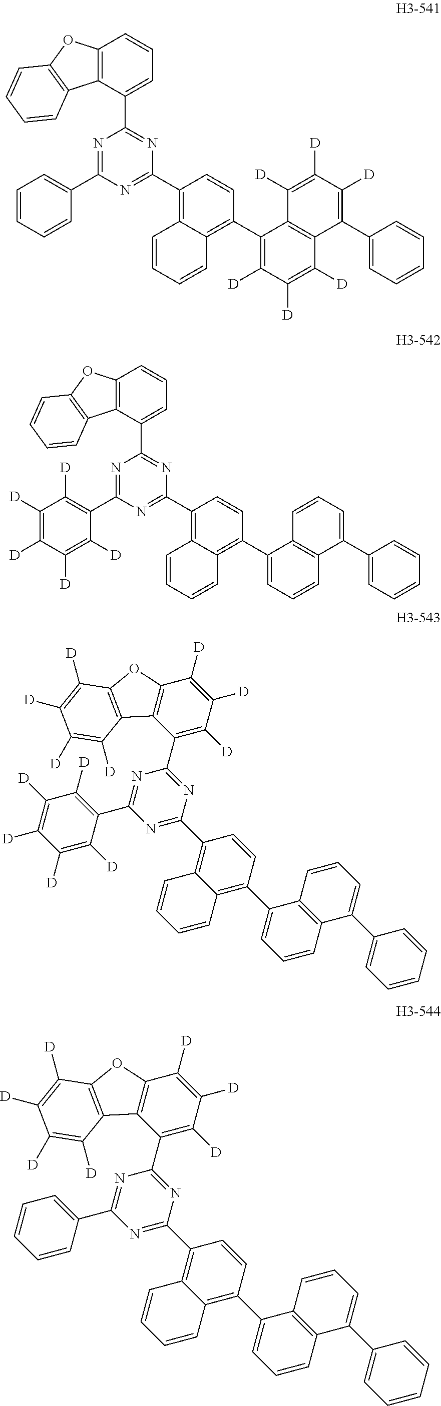

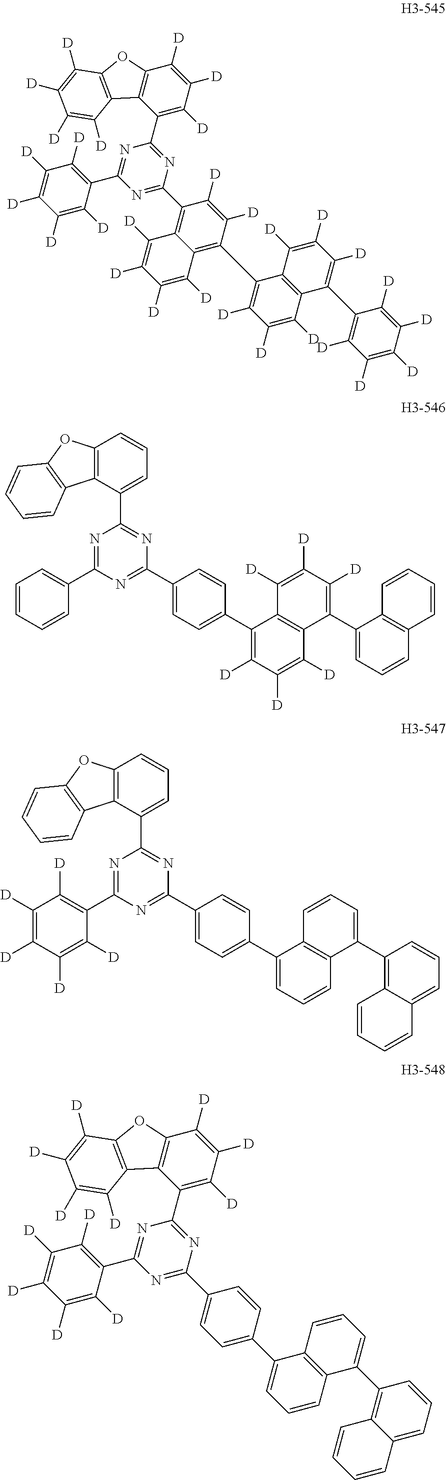

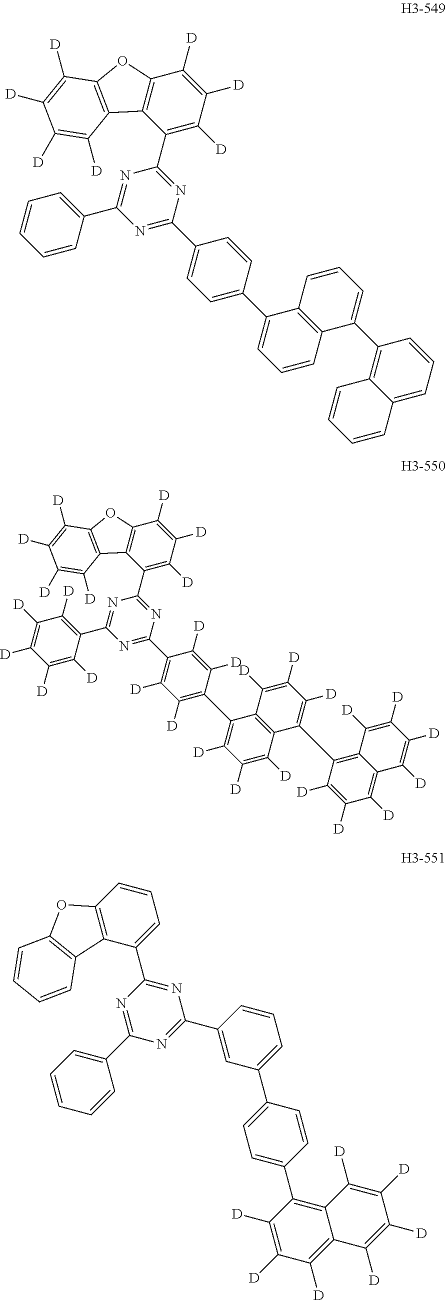

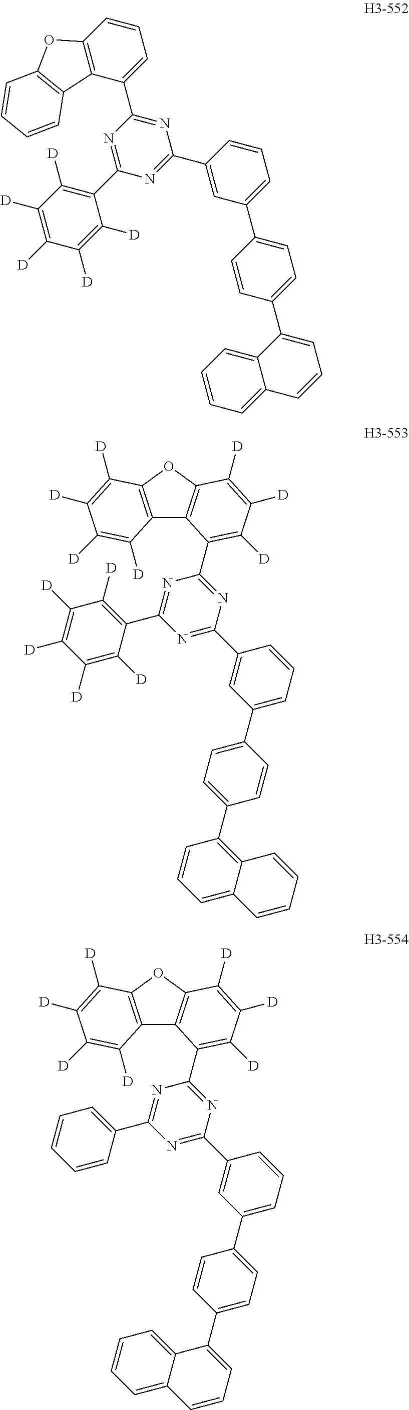

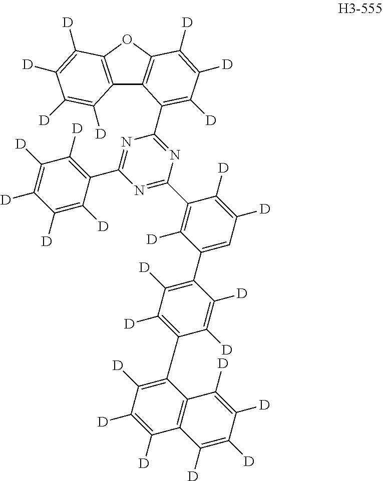 [SMILES: [2H]c1cc(-c2c([2H])c([2H])c(-c3c([2H])c([2H])c([2H])c4c([2H])c([2H])c([2H])c([2H])c34)c([2H])c2[2H])c([2H])c(-c2nc(-c3c([2H])c([2H])c([2H])c([2H])c3[2H])nc(-c3c([2H])c([2H])c([2H])c4oc5c([2H])c([2H])c([2H])c([2H])c5c34)n2)c1[2H]]

In the compounds above, Dₙ means that n number of hydrogens are replaced with deuterium, and n represents an integer from 1 to the maximum number of hydrogens in the compound.

The compounds represented by Formula 7 according to the present disclosure may be synthesized by referring to Korean Patent Application Laid-Open Nos. 2021-0124018 (Oct. 14, 2021) and 2021-0006283 (Jan. 18, 2021), but are not limited thereto.

The light-emitting layer of the organic electroluminescent device may further comprise an additional compound different from the compounds represented by Formula 6 and Formula 7.

The light-emitting layer according to another embodiment of the present disclosure may further comprise a third host compound in addition to the first host compound and the second host compound.

The organic layer may further comprise at least one layer selected from a hole injection layer, an electron transport layer, an electron injection layer, an interlayer, a hole-blocking layer, and an electron buffer layer, in addition to a hole transport layer, a light-emitting layer, a hole auxiliary layer, an electron-blocking layer, and a light-emitting auxiliary layer. The organic layer may additionally comprise an amine-based compound and/or an azine-based compound in addition to the compound of the present disclosure. Specifically, the hole injection layer, the hole transport layer, the hole auxiliary layer, the light-emitting layer, the light-emitting auxiliary layer, or the electron-blocking layer may comprise an amine-based compound, for example, an arylamine-based compound, a styrylarylamine-based compound, etc., as a hole injection material, a hole transport material, a hole auxiliary material, a light-emitting material, a light-emitting auxiliary material, and an electron-blocking material. In addition, the electron transport layer, the electron injection layer, the electron buffer layer and the hole-blocking layer may comprise an azine-based compound as an electron transport material, an electron injection material, an electron buffer material, and a hole-blocking material. In addition, the organic material layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising the metal.

The compound according to one embodiment of the present disclosure may be used as a light-emitting material for a white organic light-emitting device. The white organic light-emitting device has been suggested to have various structures such as a side-by-side arrangement method, a stacking arrangement method, or a color conversion material (CCM) method, etc. according to the arrangement of R (red), G (green) or YG (yellowish green), and B (blue) light-emitting units. In addition, the organic electroluminescent compound according to one embodiment of the present disclosure may also be used in an organic electroluminescent device comprising a quantum dot (QD).

One of the first and second electrodes may be an anode, and the other may be a cathode. The first electrode and the second electrode may each be formed with a transmissive conductive material, a transflective conductive material, or a reflective conductive material. The organic electroluminescent device may be a top emission type, a bottom emission type, or both-sides emission type depending on the type of the material forming the first electrode and the second electrode.

A hole injection layer, a hole transport layer, an electron-blocking layer, or a combination thereof may be used between an anode and a light-emitting layer. The hole injection layer may be multi-layered in order to lower the hole injection barrier (or hole injection voltage) from the anode to the hole transport layer or the electron-blocking layer, wherein two compounds may be simultaneously used in each of the multi-layers. In addition, the hole injection layer may be further doped with a p-dopant. The electron-blocking layer may be placed between the hole transport layer (or hole injection layer) and the light-emitting layer, and can confine the excitons within the light-emitting layer by blocking the overflow of electrons from the light-emitting layer to prevent light-emitting leakage. The hole transport layer or the electron transport layer may also be multi-layers, wherein each of the multi-layers may use a plurality of compounds.

An electron buffer layer, a hole-blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole-blocking layer may be placed between the electron transport layer (or electron injection layer) and the light-emitting layer, and blocks the arrival of holes to the cathode, thereby improving the probability of recombination of electrons and holes in the light-emitting layer. The hole-blocking layer or the electron transport layer may also be multi-layers, wherein each layer may use a plurality of compounds. Also, the electron injection layer may be doped as an n-dopant.

A light-emitting auxiliary layer may be a layer placed between an anode and a light-emitting layer, or between a cathode and a light-emitting layer. When placed between the anode and the light-emitting layer, the light-emitting auxiliary layer may be used to facilitate hole injection and/or hole transport or to block the overflow of electrons. When placed between the cathode and the light-emitting layer, the light-emitting auxiliary layer may be used to facilitate electron injection and/or electron transport or to block the overflow of holes. In addition, the hole auxiliary layer may be placed between the hole transport layer (or hole injection layer) and the light-emitting layer, and may exhibit an effect of facilitating or blocking the hole transport rate (or hole injection rate), and accordingly may adjust the charge balance. When an organic electroluminescent device includes two or more hole transport layers, the hole transport layer, which is further included, may be used as a hole auxiliary layer or an electron-blocking layer. The light-emitting auxiliary layer, the hole auxiliary layer, or the electron-blocking layer may have an effect of improving the efficiency and/or lifetime of the organic electroluminescent device.

In the organic electroluminescent device of the present disclosure, it is preferable to dispose at least one layer selected from a chalcogenide layer, a metal halide layer, and a metal oxide layer (hereinafter referred to as a “surface layer”) on at least one inner surface of a pair of electrodes. Specifically, a chalcogenide (including an oxide) layer of silicon and aluminum is preferably placed on an anode surface of an electroluminescent medium layer side, and a metal halide layer or a metal oxide layer is preferably placed on a cathode surface of an electroluminescent medium layer side. Driving stabilization of the organic electroluminescent device can be obtained by the surface layer. Preferred examples of the chalcogenide include SiO_{X} (1≤X≤2), AlO_{X} (1≤X≤1.5), SiON, SiAlON, etc., preferred examples of the metal halide include LiF, MgF₂, CaF₂, a rare earth metal fluoride, etc., and preferred examples of the metal oxide include Cs₂O, Li₂O, MgO, SrO, BaO, CaO, etc.

In addition, in an organic electroluminescent device of the present disclosure, a mixed region of an electron transport compound and a reductive dopant, or a mixed region of a hole transport compound and an oxidative dopant, may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferred oxidative dopants include various Lewis acids and acceptor compounds, and preferred reductive dopants include alkali metals, alkali metal compounds, alkaline earth metals, rare earth metals, and mixtures thereof. In addition, an organic electroluminescent device having at least two light-emitting layers and emitting white light may be manufactured by using the reductive dopant layer as a charge-generation layer.

An organic electroluminescent device according to one embodiment of the present disclosure may be an organic electroluminescent device having a tandem structure. In the case of the tandem organic electroluminescent device according to one embodiment, a single light-emitting unit (light-emitting part) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer stacked between the first and second electrodes and which emits light in a specific wavelength range. It may include a plurality of light-emitting units, and each of the light-emitting units may include a hole transport zone, a light-emitting layer, and an electron transport zone, the hole transport zone may include a hole injection layer and a hole transport layer, and the electron transport zone may include an electron transport layer and an electron injection layer. According to one embodiment of the present disclosure, three or more light-emitting layers may be included in the light-emitting unit. A plurality of light-emitting units may emit the same color or different colors. In addition, one light-emitting unit may include one or more light-emitting layers, and the plurality of light-emitting layers may be light-emitting layers of the same or different colors. It may include one or more charge generation layers located between each of the light-emitting units. The charge generation layer refers to the layer in which holes and electrons are generated when voltage is applied. When there are three or more light-emitting units, a charge generation layer may be located between each light-emitting unit. The plurality of charge generation layers may be the same as or different from one another. By disposing the charge generation layer between light-emitting units, current efficiency is increased in each light-emitting unit, and charges can be smoothly distributed. Specifically, the charge generation layer is provided between two adjacent stacks and can serve to drive a tandem organic electroluminescent device using only a pair of an anode and a cathode without a separate internal electrode located between the stacks.

The charge generation layer may be composed of an N-type charge generation layer and a P-type charge generation layer, and the N-type charge generation layer may be doped with an alkali metal, an alkaline earth metal, or a compound of an alkali metal and an alkaline earth metal. The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The P-type charge generation layer may be made of a metal or an organic material doped with a P-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the P-type dopant and host materials used in the P-type doped organic material.

The organic electroluminescent device according to one embodiment of the present disclosure may further comprise one or more dopants in the light-emitting layer. The dopant comprised in the organic electroluminescent device of the present disclosure may be at least one phosphorescent or fluorescent dopant, and is preferably a phosphorescent dopant. The phosphorescent dopant material applied to the organic electroluminescent device of the present disclosure is not particularly limited, but may be preferably selected from the group consisting of the metallated complex compounds of iridium (Ir), osmium (Os), copper (Cu), and platinum (Pt), more preferably selected from the group consisting of ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may be a compound represented by the following Formula 101 or 102, but is not limited thereto.

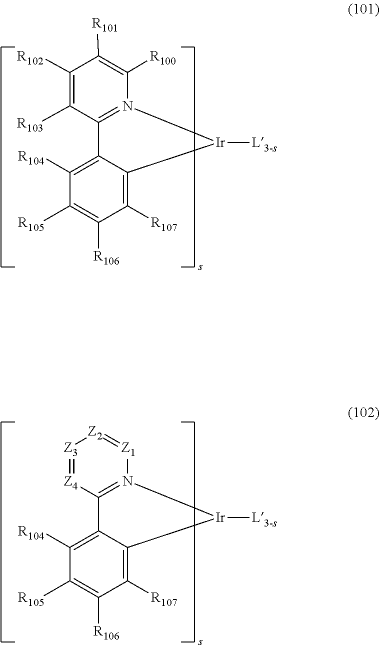

In Formulas 101 and 102,
L¹ is any one selected from the following Structures 1 to 3:

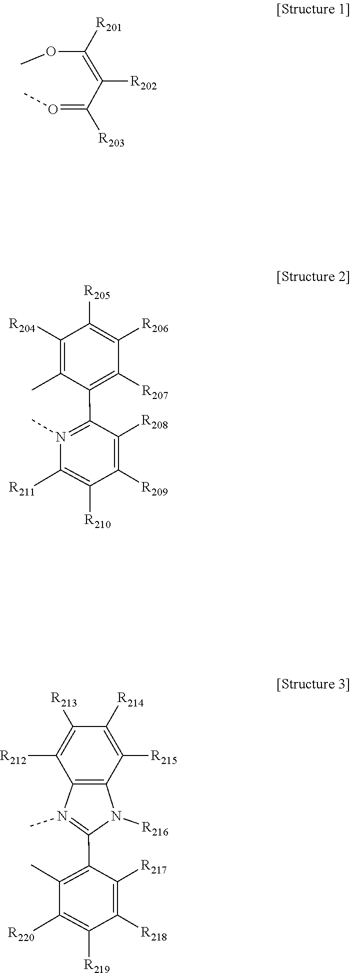

R₁₀₀ to R₁₀₃ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent(s) to form a ring(s), for example, to form a ring(s) with a pyridine, e.g., a substituted or unsubstituted quinoline, a substituted or unsubstituted isoquinoline, a substituted or unsubstituted thienopyridine, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent, hydrogen, deuterium, halogen, deuterium- and/or halogen-substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, cyano, a substituted or unsubstituted (C1-C30)alkoxy, or a substituted or unsubstituted di(C1-C30)alkylamino; or may be linked to an adjacent substituent(s) to form a ring(s), for example, to form a ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent, hydrogen, deuterium, halogen, deuterium- and/or halogen-substituted or unsubstituted (C1-C30)alkyl, a substituted or unsubstituted (C3-C30)cycloalkyl, a substituted or unsubstituted (C6-C30)aryl, a substituted or unsubstituted (C1-C30)alkoxy, or a substituted or unsubstituted di(C1-C30)alkylamino; or may be linked to an adjacent substituent(s) to form a ring(s); for example, to form a substituted or unsubstituted benzene, a substituted or unsubstituted fluorene, a substituted or unsubstituted benzofuran, a substituted or unsubstituted benzothiophene, a substituted or unsubstituted dibenzofuran, a substituted or unsubstituted dibenzothiophene, a substituted or unsubstituted furopyridine, or a substituted or unsubstituted thiophene;
Z₁ to Z₄ each independently represent N or CK₁;
K₁ each independently represent hydrogen, deuterium, halogen, deuterium- and/or halogen-substituted or unsubstituted (C1-C30)alkyl, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or may be linked to an adjacent substituent(s) to form a ring(s); for example, to form a substituted or unsubstituted benzene, a substituted or unsubstituted naphthalene, a substituted or unsubstituted thiophene, a substituted or unsubstituted benzothiophene, a substituted or unsubstituted fluorene, a substituted or unsubstituted dibenzofuran, or a substituted or unsubstituted dibenzothiophene; and
s represents an integer of 1 to 3.

Specifically, the specific examples of the dopant compound include the following, but are not limited thereto.

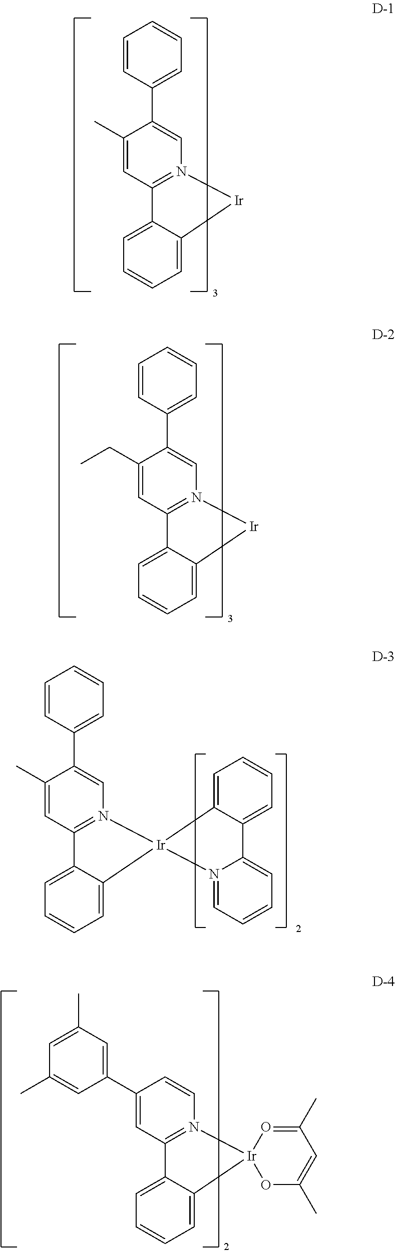

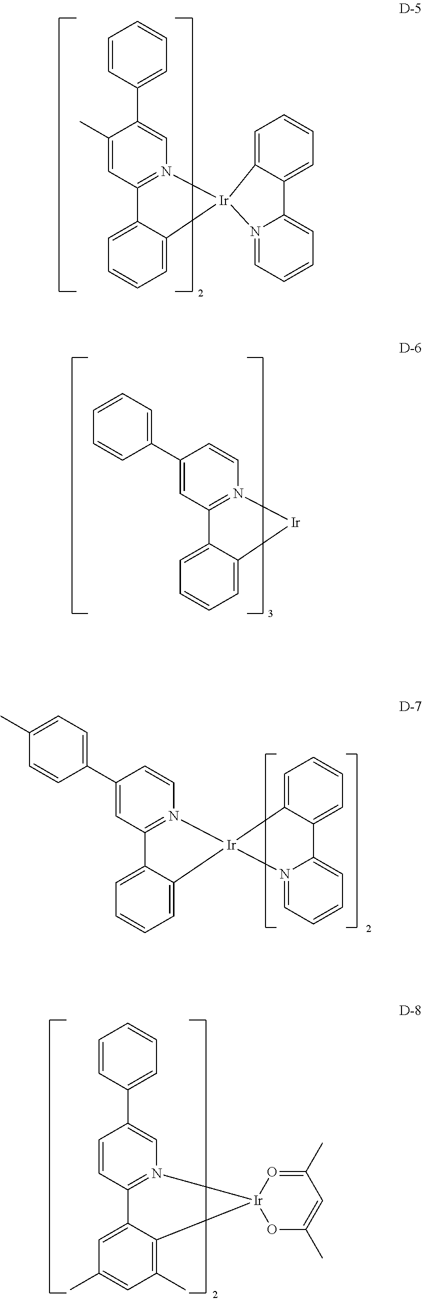

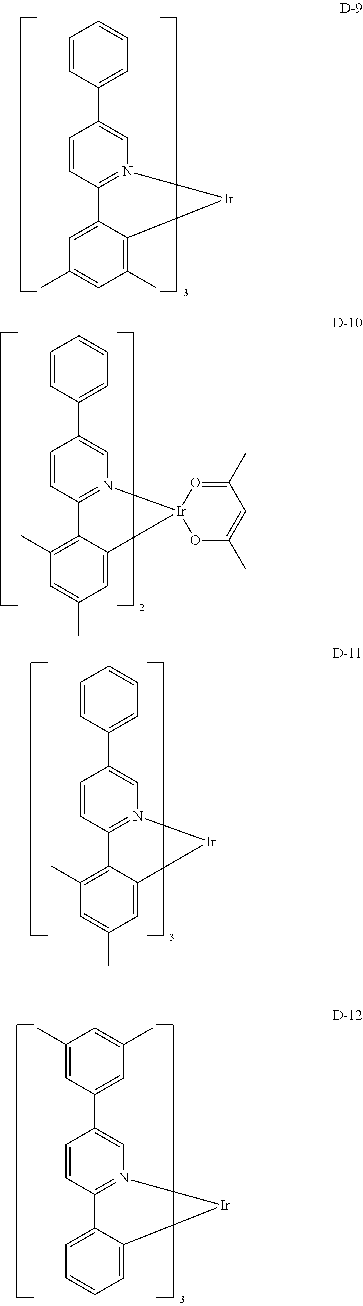

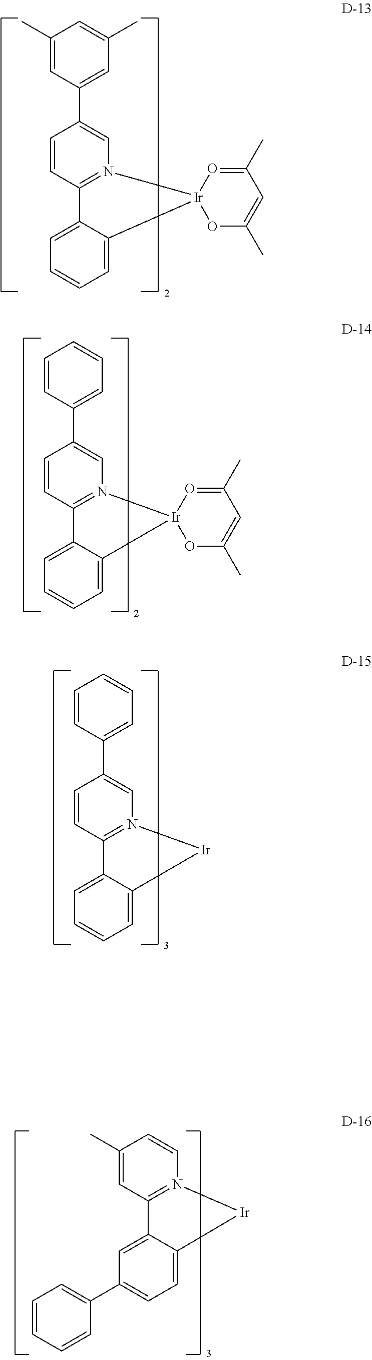

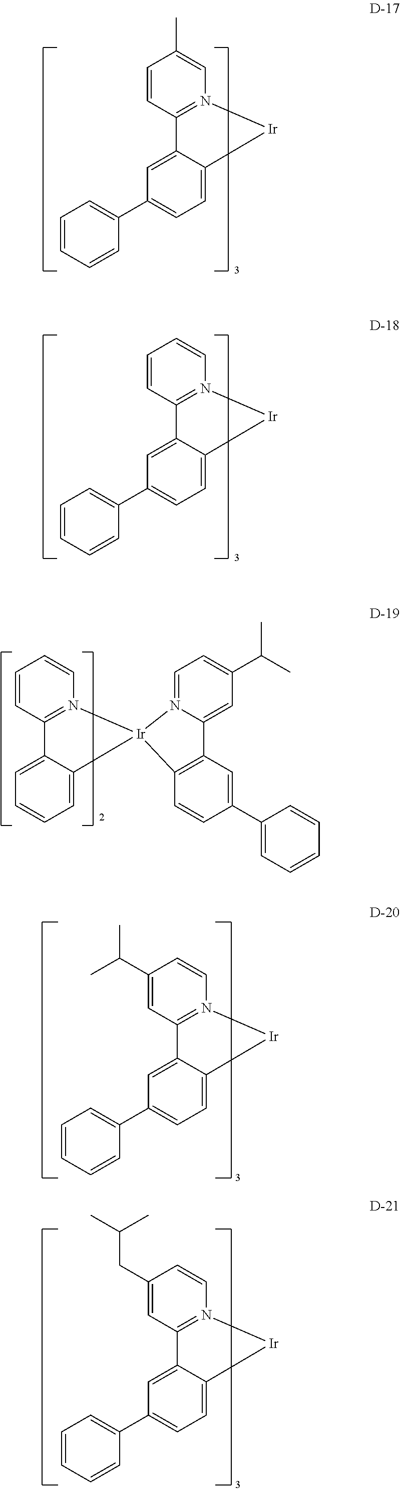

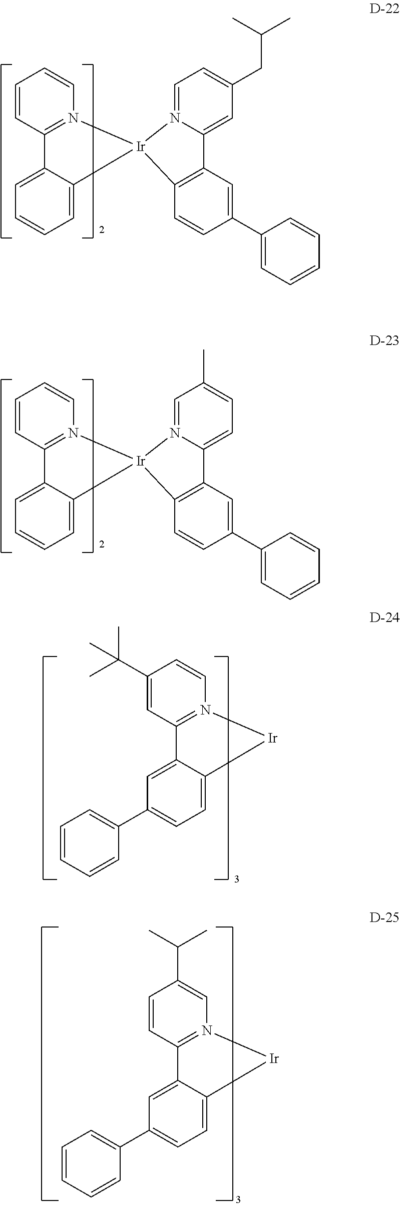

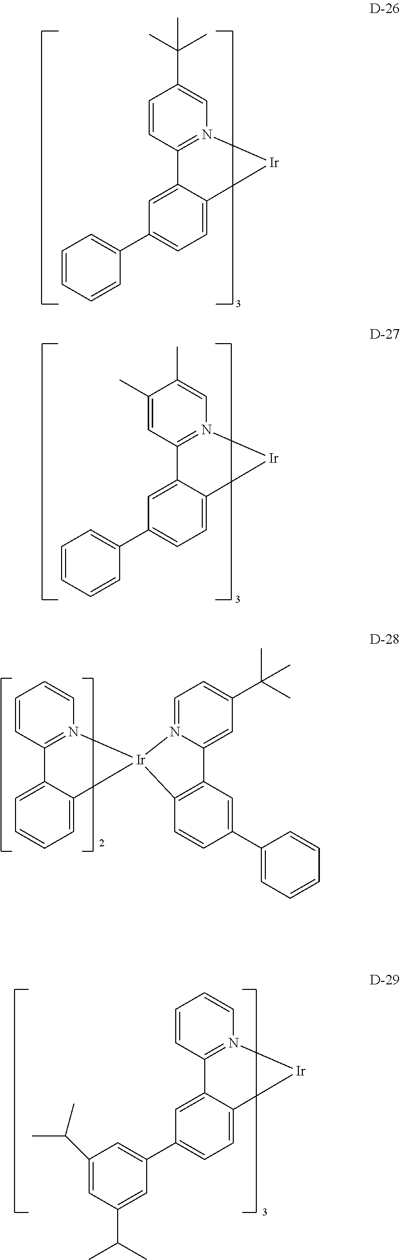

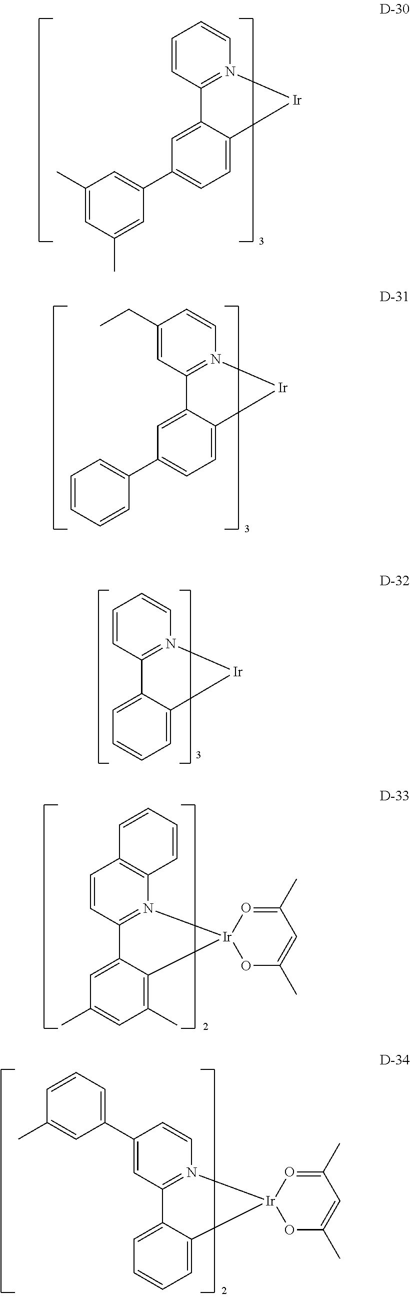

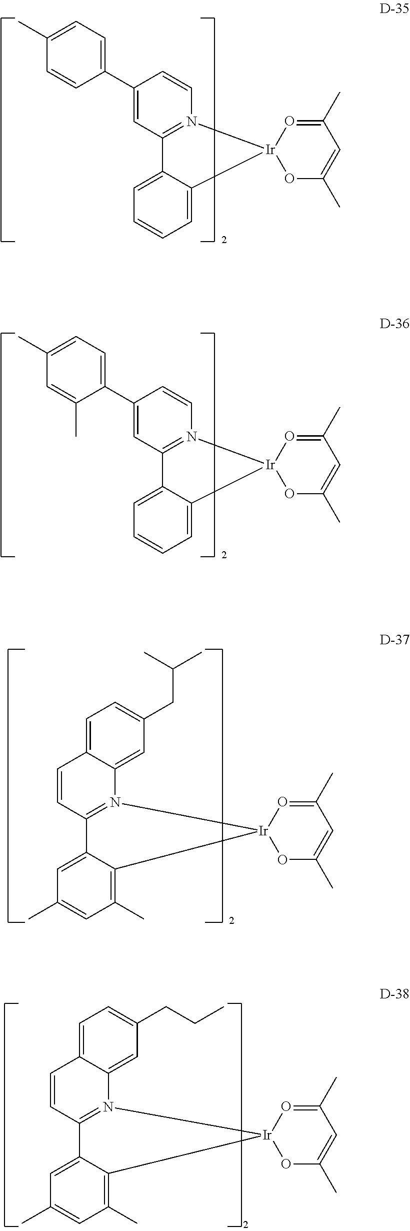

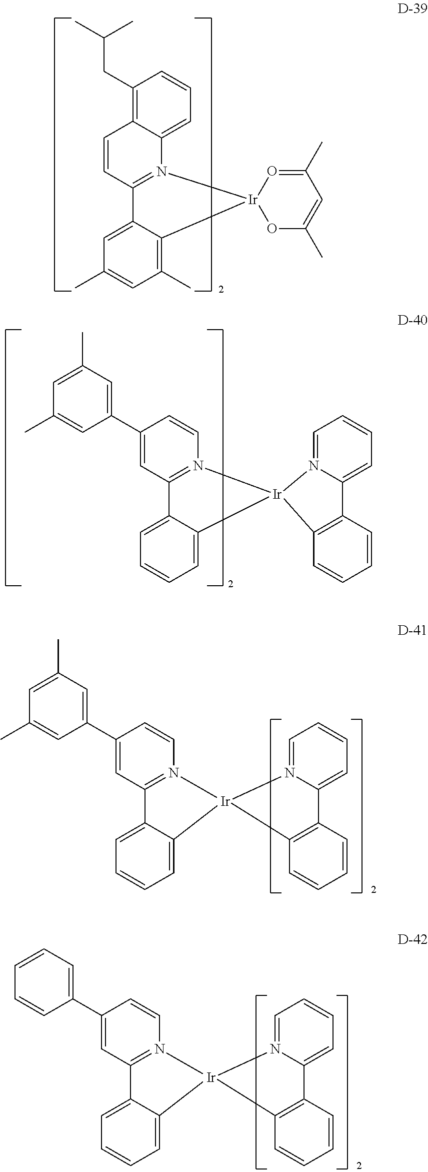

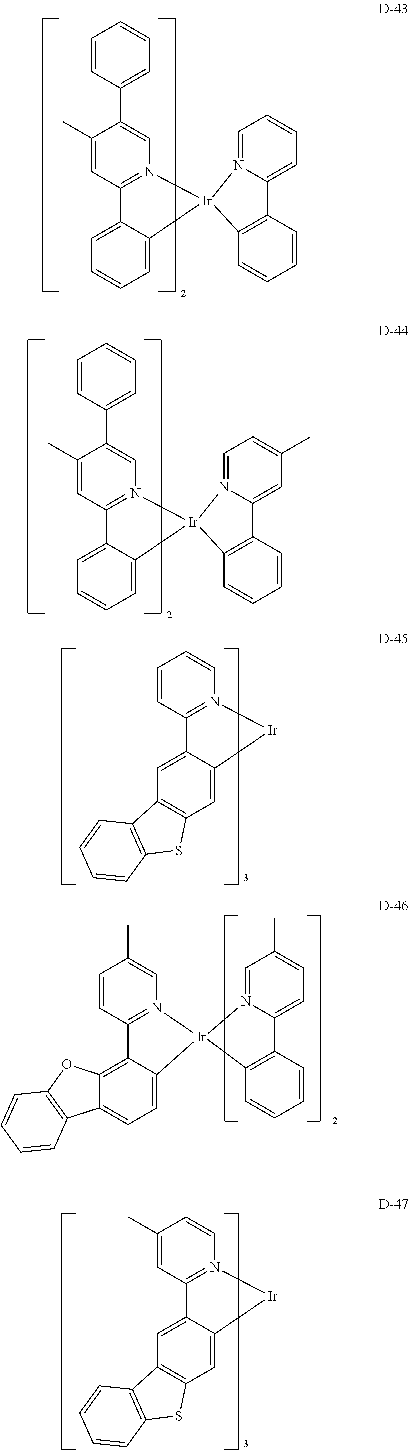

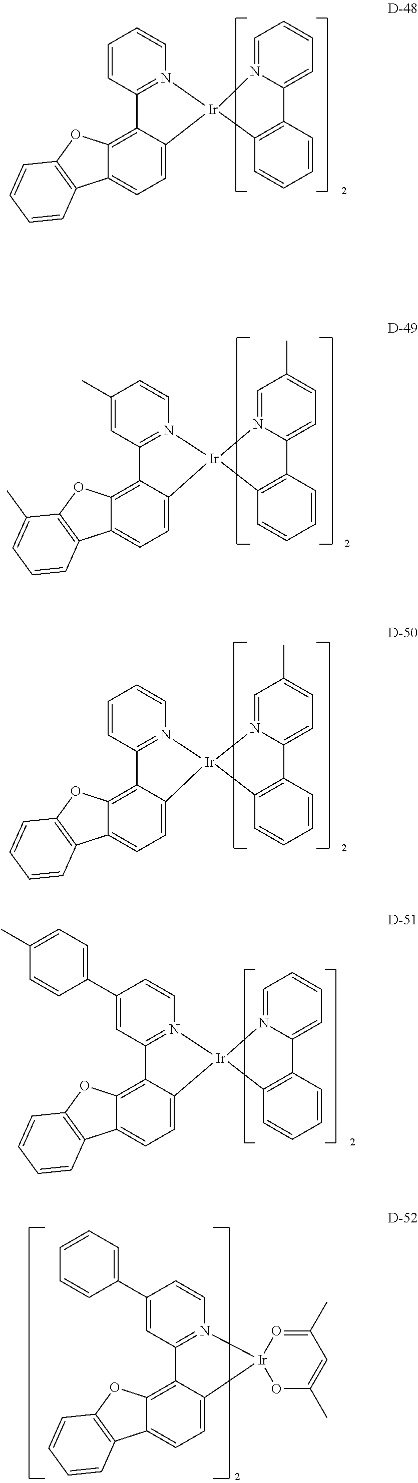

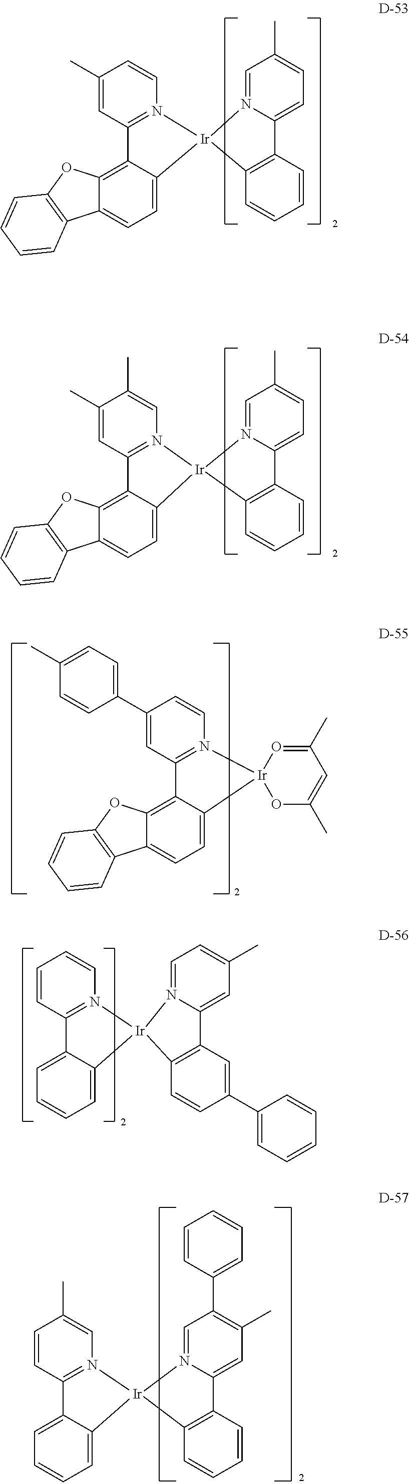

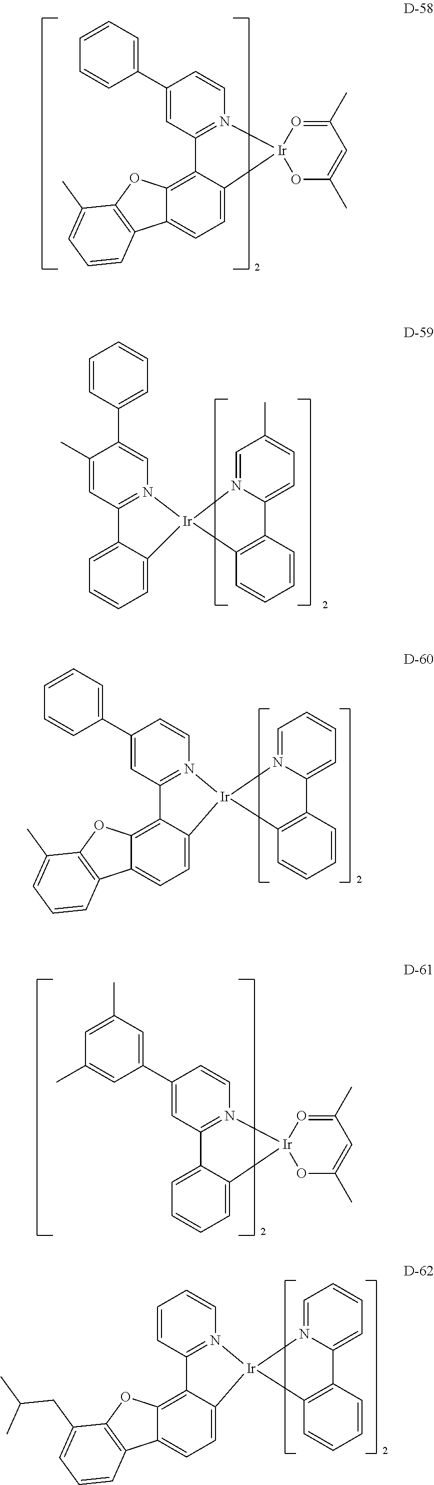

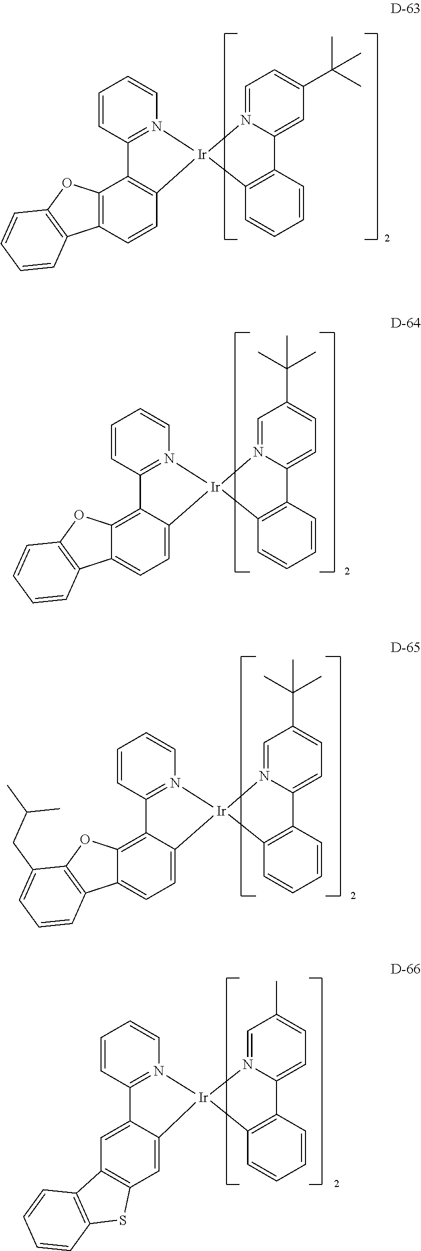

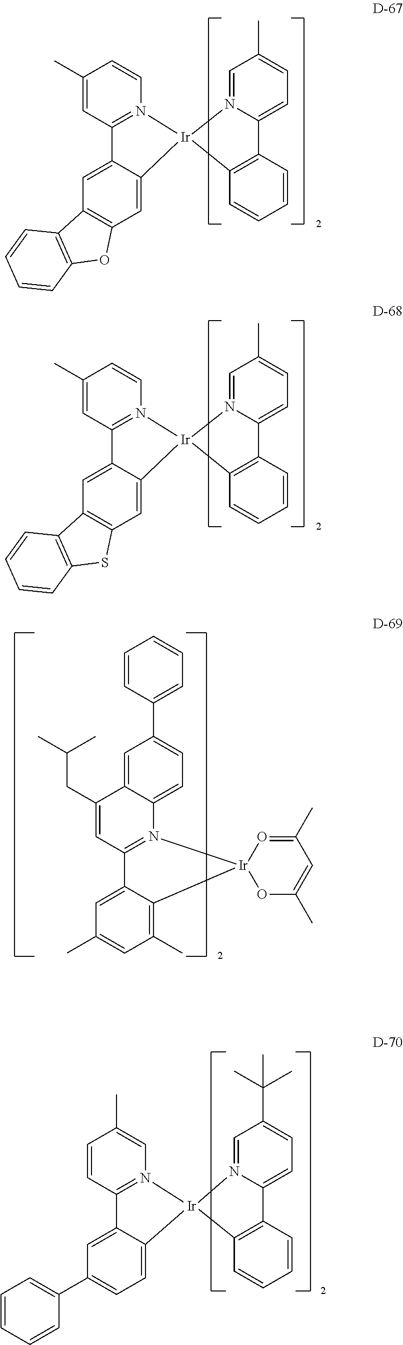

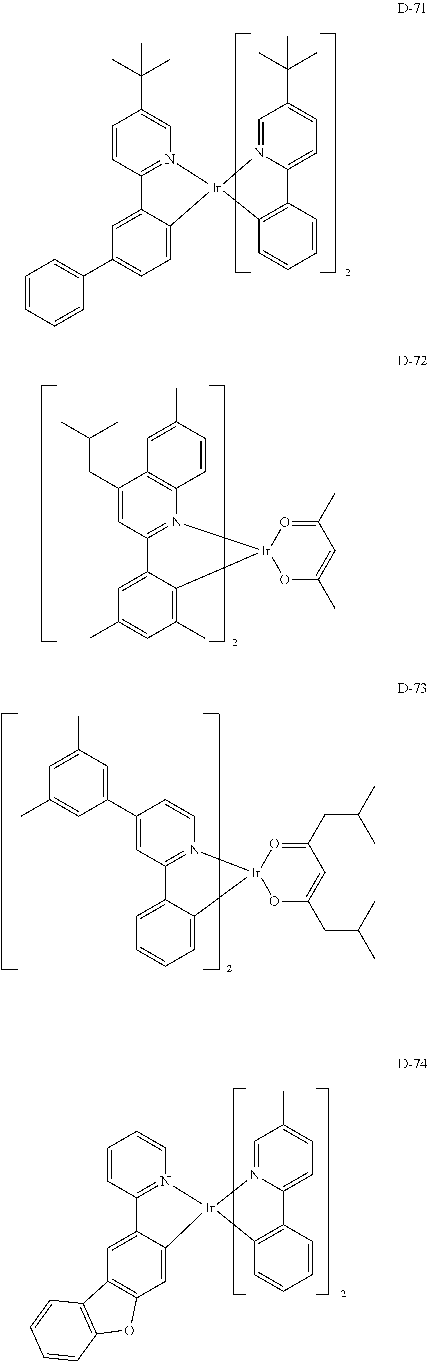

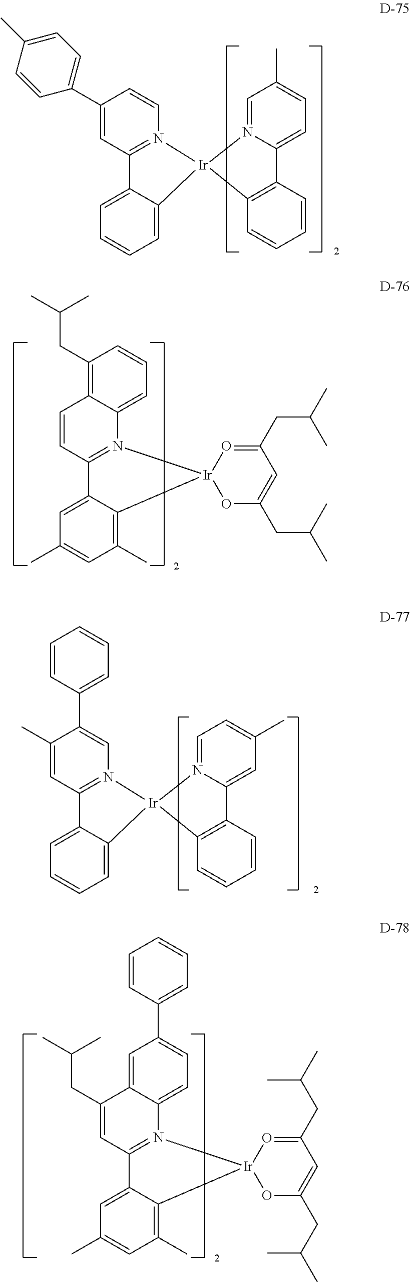

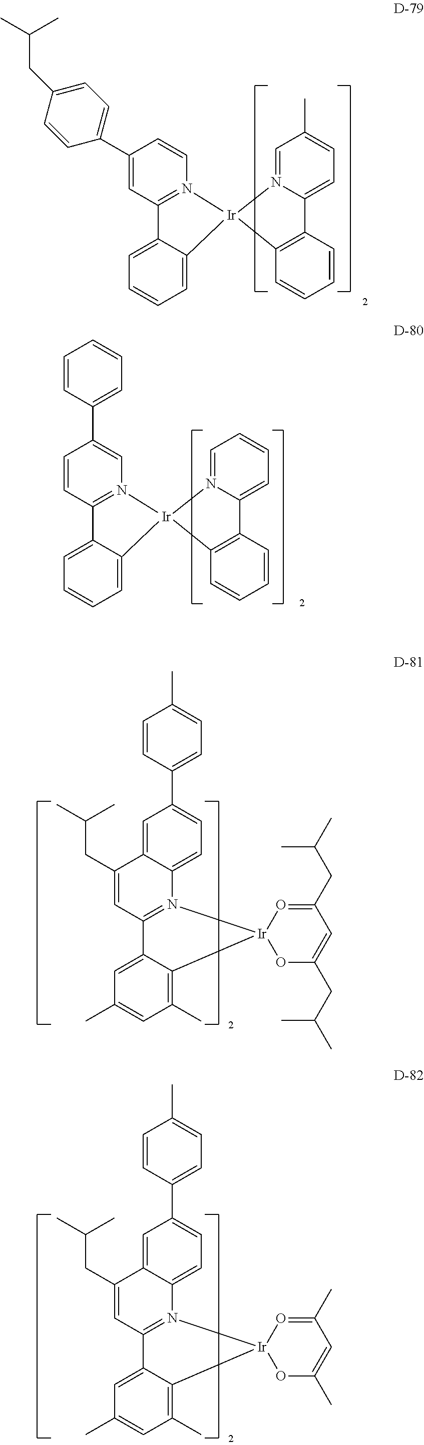

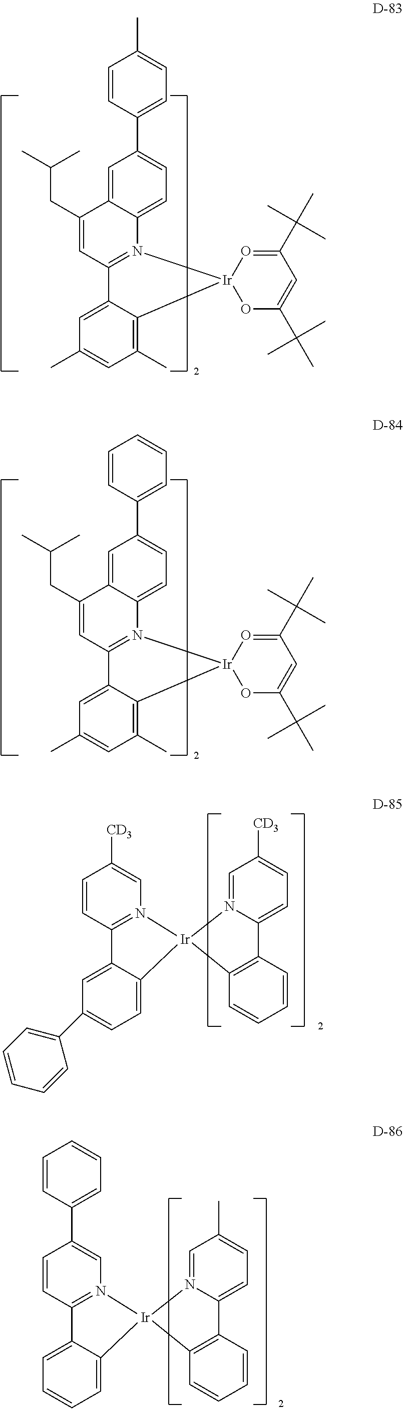

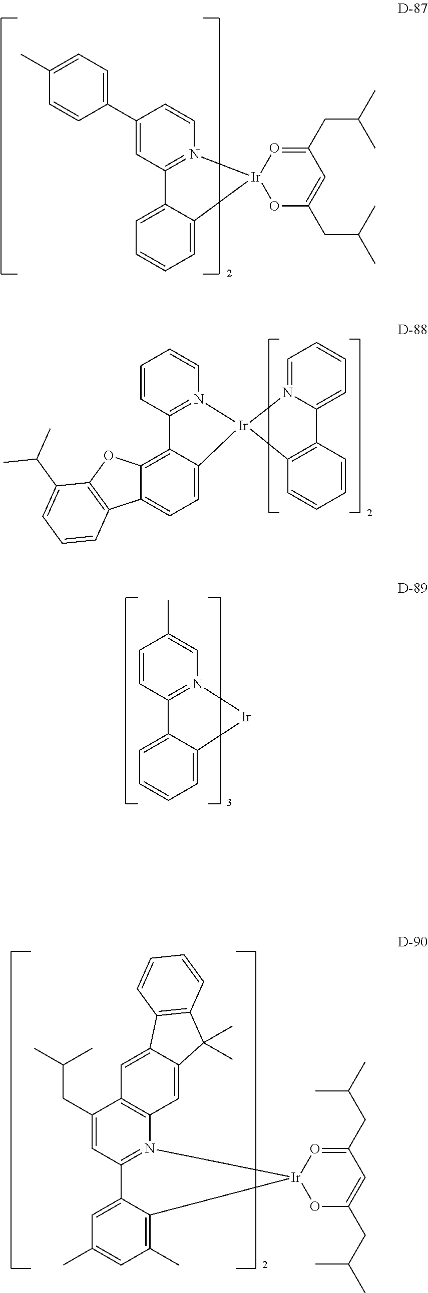

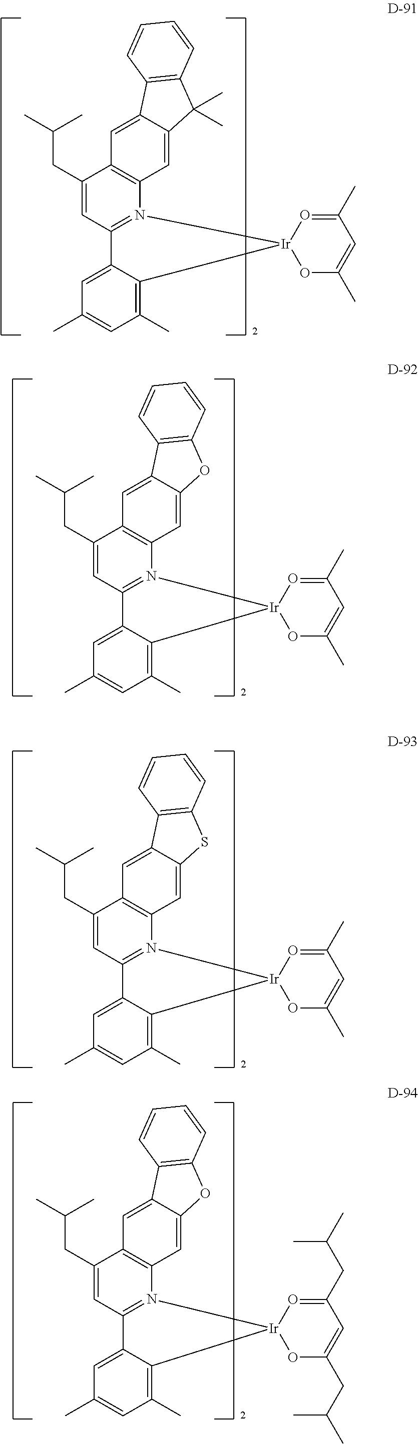

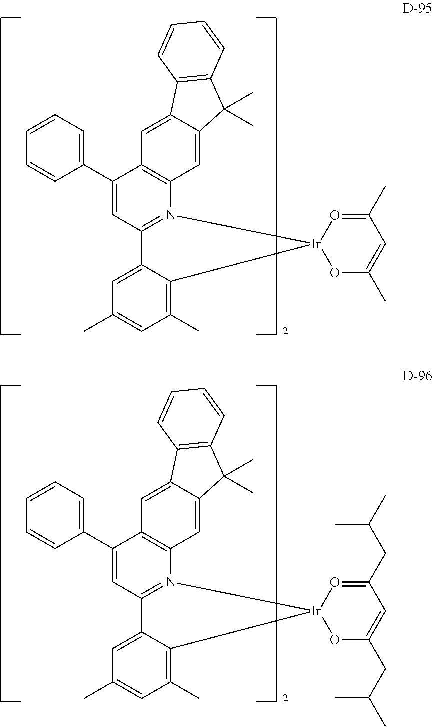

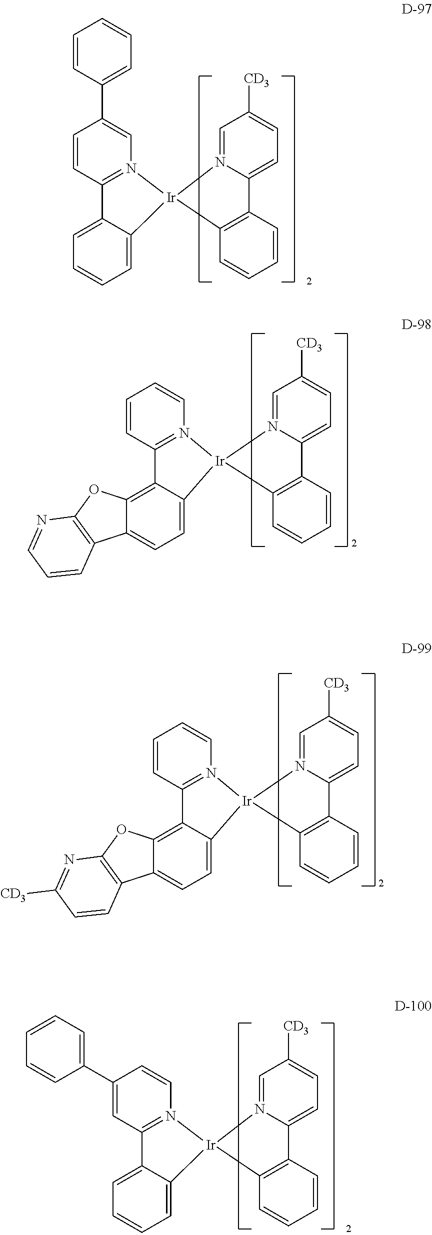

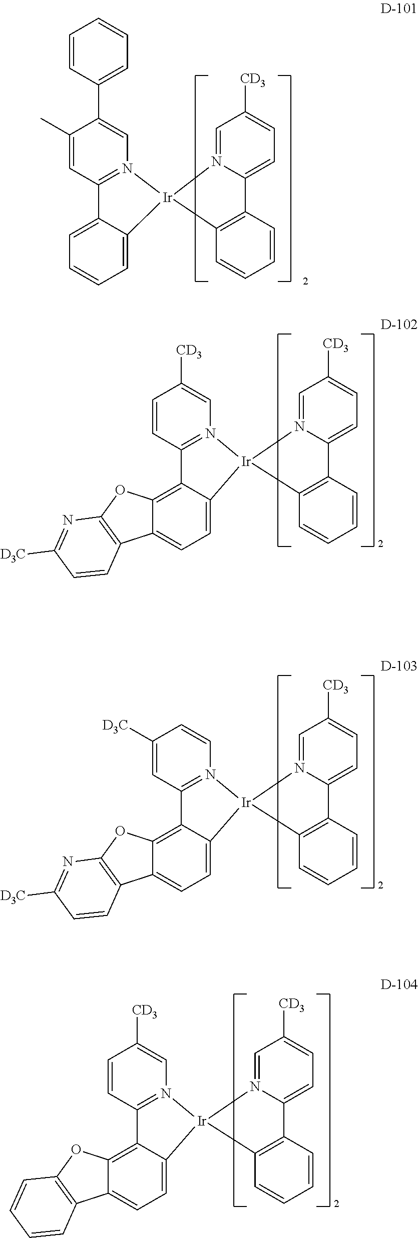

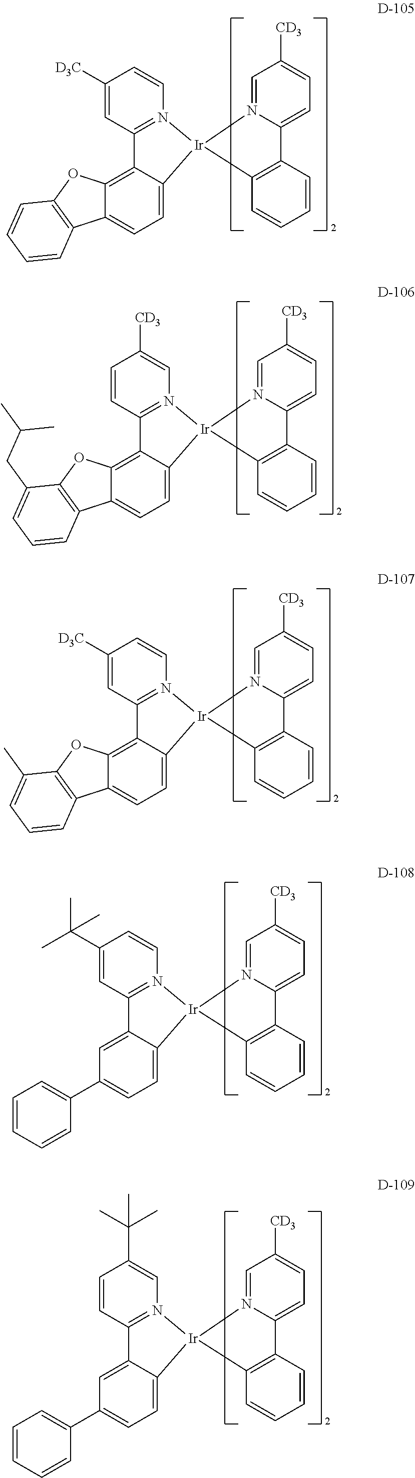

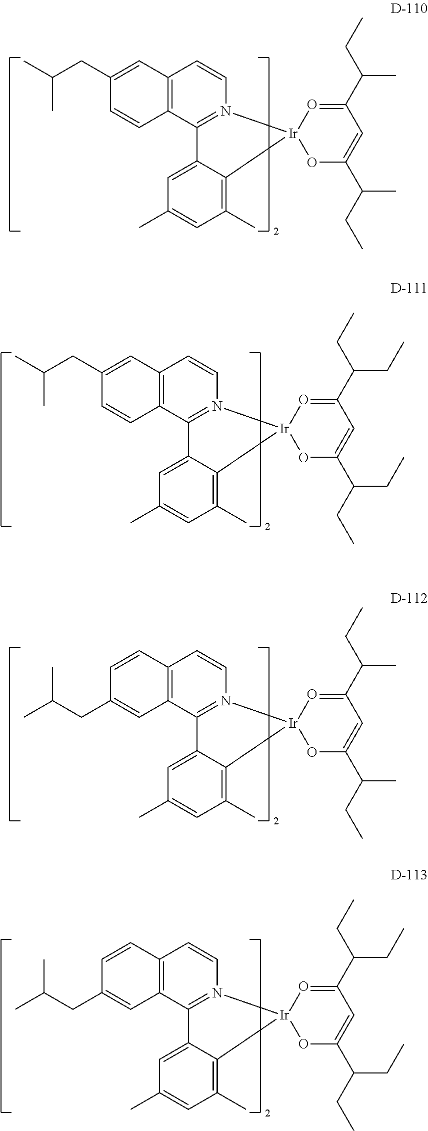

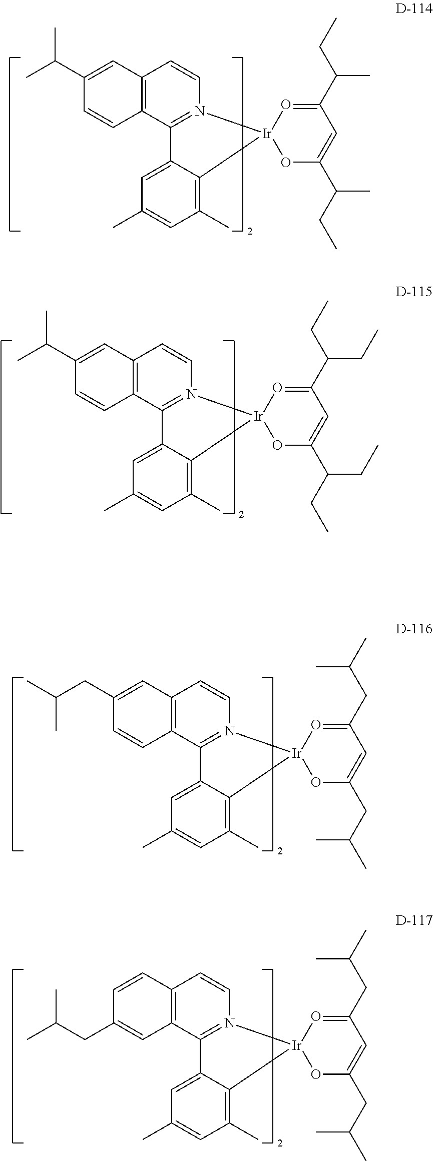

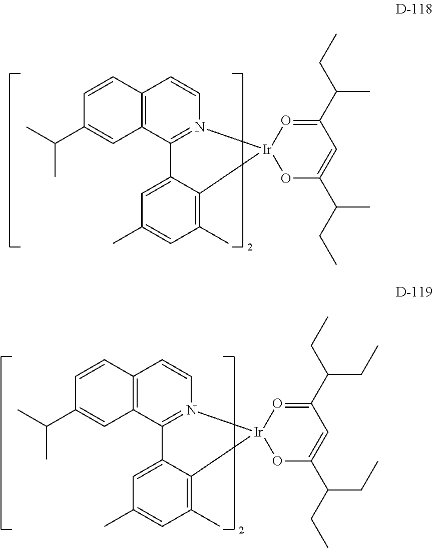

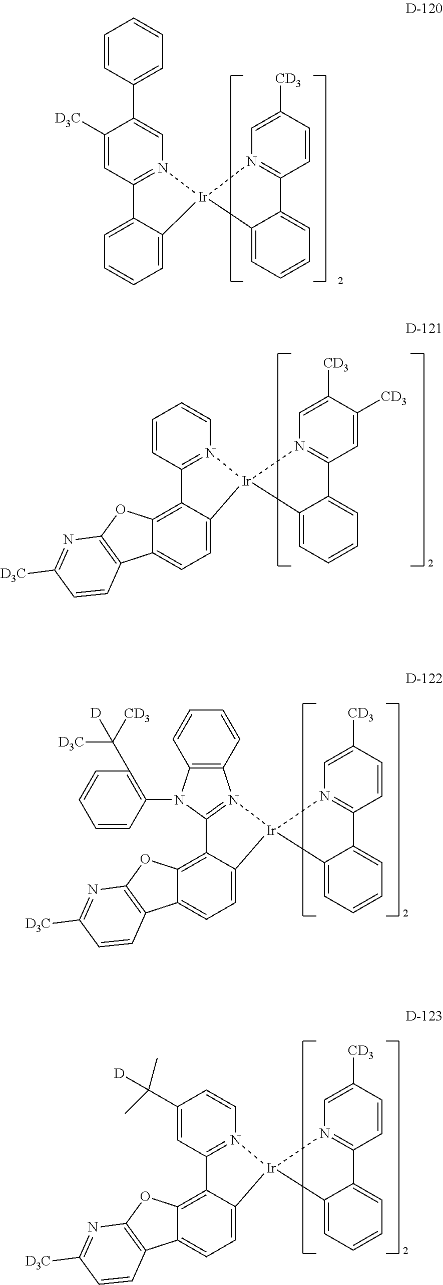

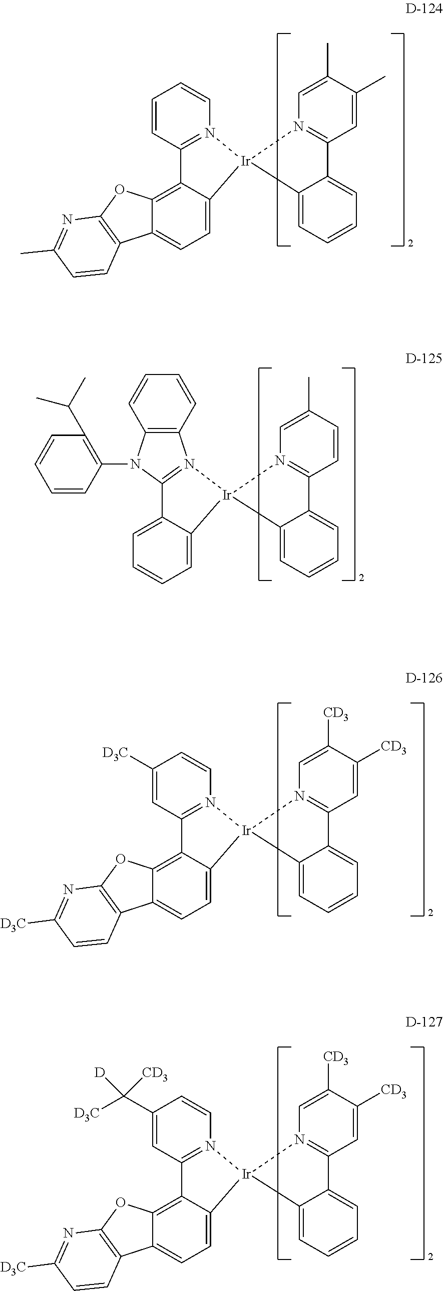

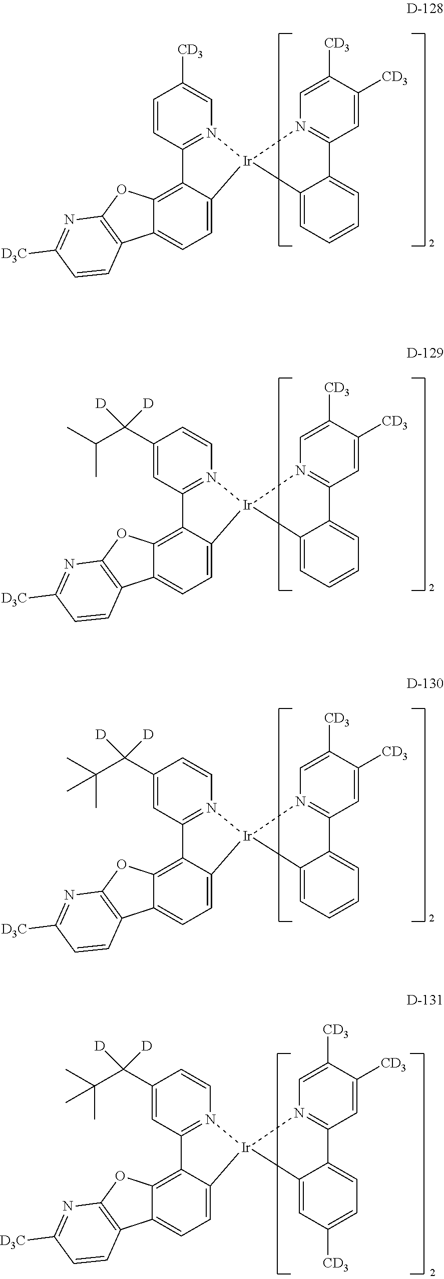

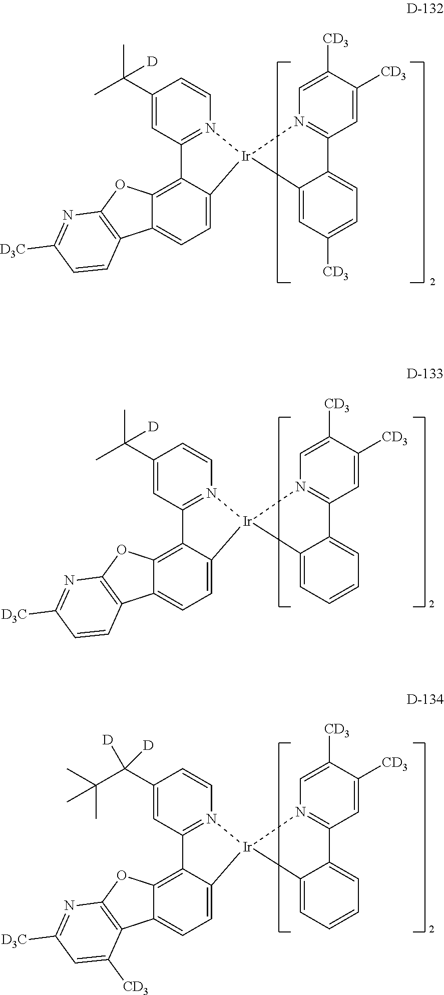

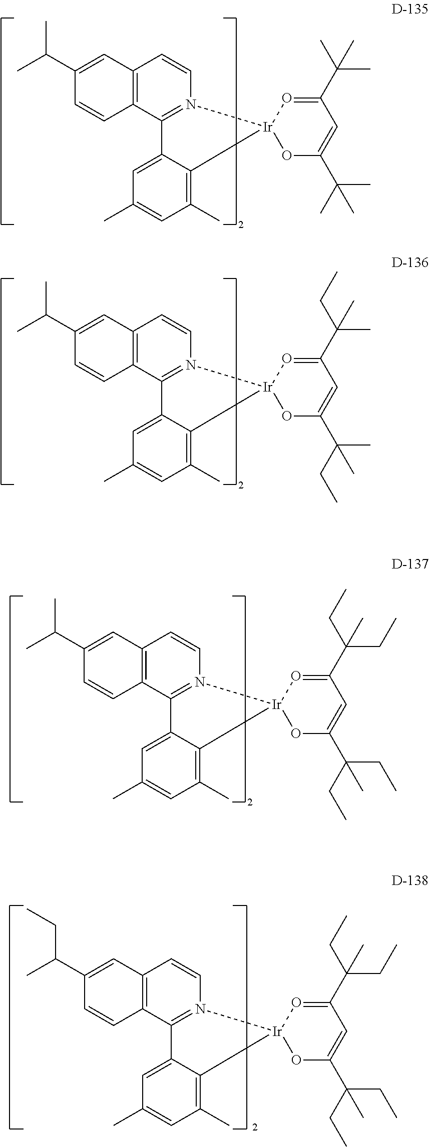

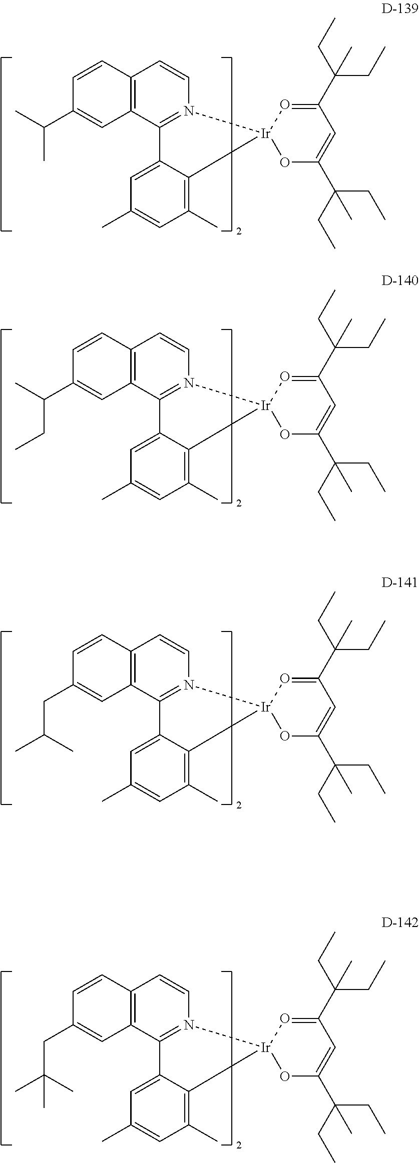

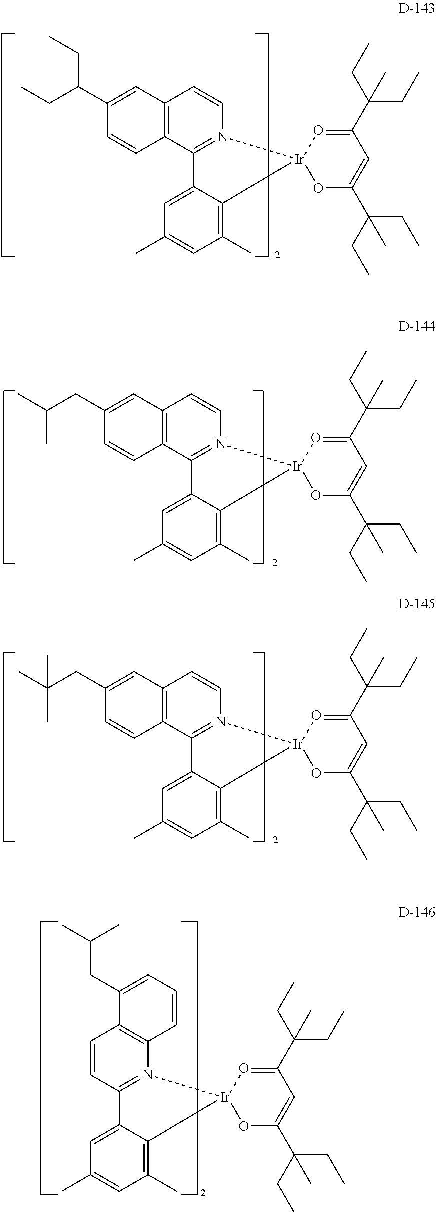

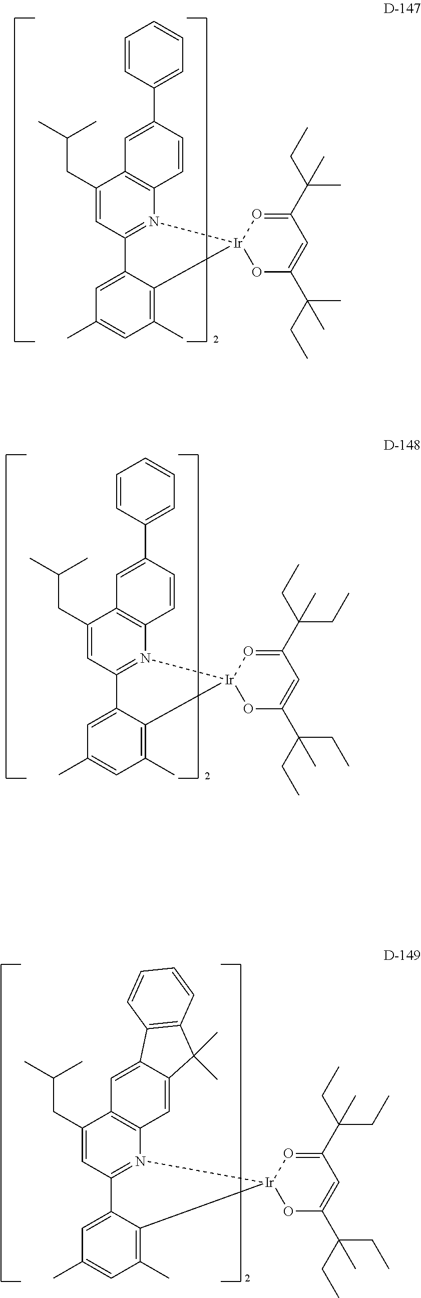

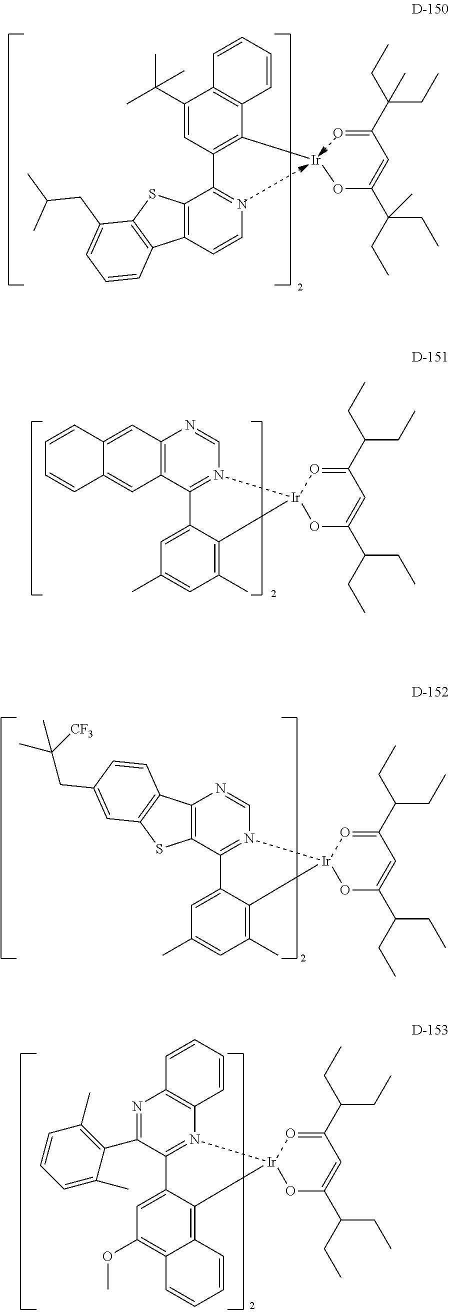

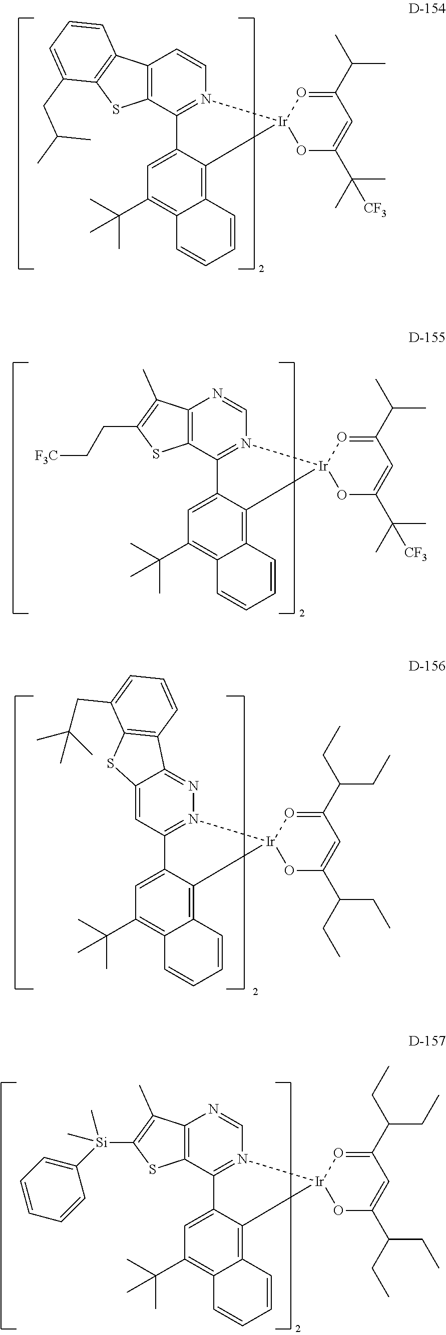

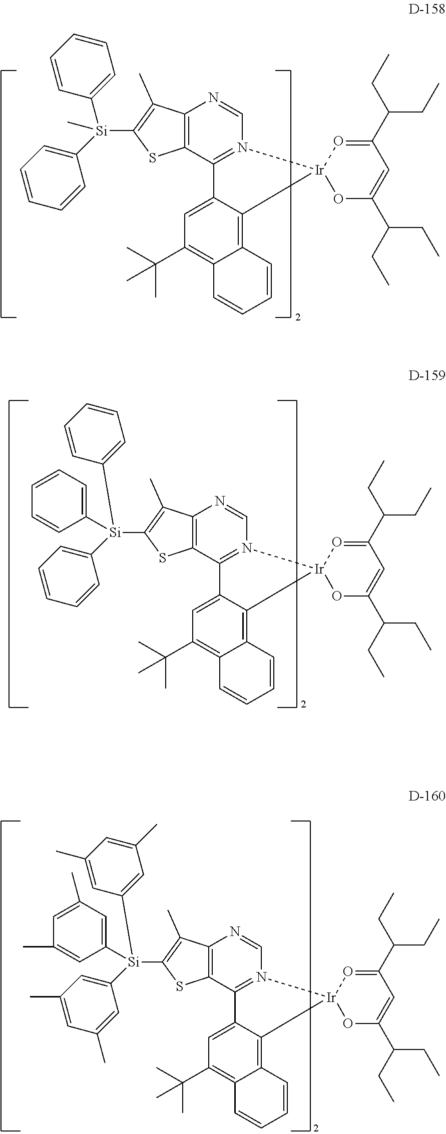

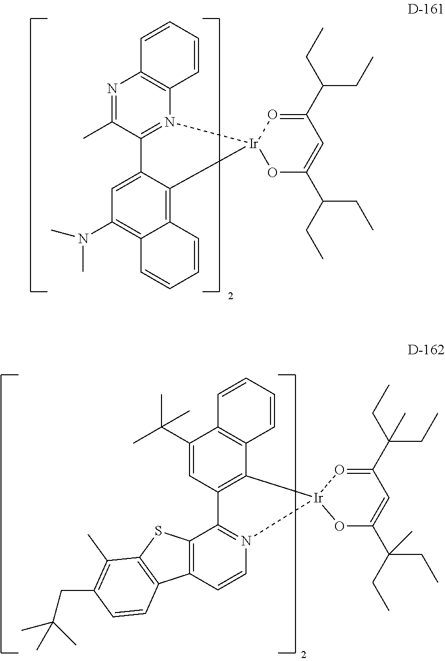

In order to form each layer of the organic electroluminescent device of the present disclosure, dry film-forming methods such as vacuum evaporation, sputtering, plasma, ion plating methods, etc. or wet film-forming methods such as spin coating, dip coating, flow coating methods, etc. can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film formation capability.

When forming a film of the electroluminescent compound according to the present disclosure, the film can be formed by the above-listed methods, and can be formed commonly by a co-deposition or mixed deposition process. The co-deposition is a method of mixing and depositing two or more materials by placing them in separate crucible sources and applying current to two cells simultaneously to evaporate the materials, and the mixed deposition is a method of mixing two or more materials in one crucible source before deposition and then applying current to one cell to evaporate the materials.

According to one embodiment, the present disclosure can provide a display device comprising an electroluminescent compound represented by Formula 1. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of the organic electroluminescent compounds according to the present disclosure, the physical properties thereof, and the driving voltage, current efficiency, and lifetime properties of the OLED according to the present disclosure will be explained in detail. However, the following examples only describe the properties of the compound and the OLED according to the present disclosure, but the present disclosure is not limited to the following examples.

### Example 1: Preparation of Compound C-9

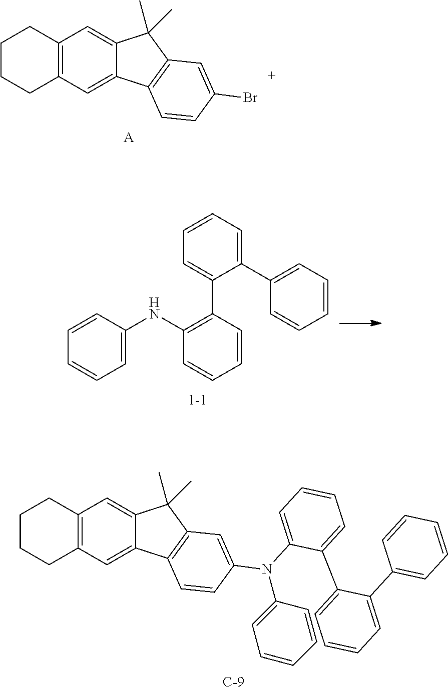

Compound A (11.1 g, 0.034 mol), Compound 1-1 (10 g, 0.031 mol), Pd₂(dba)₃ (1.5 g, 0.0017 mol), 50% P(tBu)₃ (1.68 mL), NaOtBu (6.5 g, 0.068 mol), and toluene (170 mL) were dissolved in a flask, and the mixture was refluxed at 120° C. for 2 hours. After completion of the reaction, the organic layer was extracted with methyl chloride (MC), and the remaining moisture was removed with magnesium sulfate. Thereafter, the organic layer was separated by column chromatography to obtain Compound C-9 (13.3 g, yield: 69%).

| | MW | M.P. |
|---|---|---|
| C-9 | 567.78 | 256° C. |

### Example 2: Preparation of Compound C-29

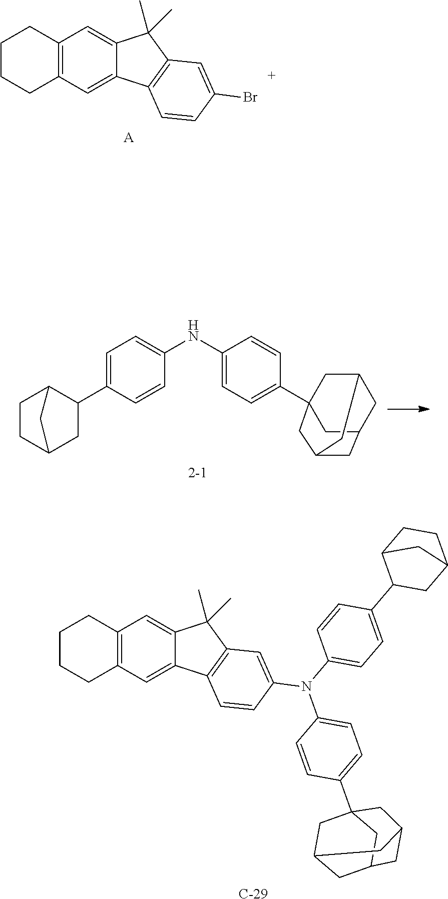

Compound A (8.8 g, 0.027 mol), Compound 2-1 (10 g, 0.025 mol), Pd₂(dba)₃ (1.24 g, 0.00135 mol), 50% P(tBu)₃ (1.3 mL), NaOtBu (5.2 g, 0.054 mol), and toluene (135 mL) were dissolved in a flask, and the mixture was refluxed at 120° C. for 2 hours. After completion of the reaction, the organic layer was extracted with MC, and the remaining moisture was removed with magnesium sulfate. Thereafter, the organic layer was separated by column chromatography to obtain Compound C-29 (7.9 g, yield: 45%).

| | MW | M.P. |
|---|---|---|
| C-29 | 643.96 | 227° C. |

### Device Examples 1-1 an 1-2: Producing Red Light-Emitting OLEDs According to the Present Disclosure

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1 was introduced into a cell of the vacuum vapor deposition apparatus, and Compound HT1-1 was introduced into another cell. The two materials were evaporated at different rates, and Compound HI-1 was deposited in a doping amount of 5 wt % based to the total amount of Compound HI-1 and Compound HT1-1 to form a hole injection layer with a thickness of 10 nm. Subsequently, Compound HT1-1 was deposited on the hole injection layer to form a first hole transport layer with a thickness of 90 nm. The compound shown in Table 1 below was introduced into another cell of the vacuum vapor deposition apparatus and was then evaporated by applying an electric current to the cell to thereby deposit a second hole transport layer with a thickness of 60 nm on the first hole transport layer. Next, Compound HT3-1 was introduced into another cell of the vacuum vapor deposition apparatus and was evaporated by applying an electric current to the cell to thereby deposit a third hole transport layer with a thickness of 7.5 nm on the second hole transport layer. After formation of the hole injection layer and the hole transport layers, a light-emitting layer was deposited thereon as follows. After Compound H1-1 and Compound H2-1 were introduced into two cells of the vacuum vapor deposition apparatus as hosts, the two compounds were evaporated at a ratio of 1:1. After Compound D-39 was introduced into another cell as a dopant, the dopant was deposited in a doping amount of 2 wt % based on the total amount of the hosts and the dopant to form a light-emitting layer with a thickness of 36 nm on the third hole transport layer. An electron buffer layer with a thickness of 5 nm was then deposited on the light-emitting layer using Compound B-1 as the electron buffer material. Next, an electron transport layer was deposited to a thickness of 25 nm on the electron buffer layer using a 2:1 weight ratio mixture of Compound ET-1 and compound EI-1 as the electron transport materials. After Compound EI-1 was deposited as an electron injection layer with a thickness of 2 nm on the electron transport layer, an Al cathode was deposited with a thickness of 80 nm on the electron injection layer using another vacuum vapor deposition apparatus to manufacture an OLED.

### Comparative Examples 1-1 and 1-2: Producing Red Light-Emitting OLEDs not According to the Present Disclosure

OLEDs were produced in the same manner as in Device Example 1-1, except that the respective compounds of the second hole transport layer described in Table 1 below were used.

The driving voltage and current efficiency of the OLED produced in Device Examples 1-1 and 1-2 and Comparative Examples 1-1 and 1-2 as described above were measured, and the results thereof are shown in the following Table 1.

TABLE 1

| | | Second | | | | |
|---|---|---|---|---|---|---|
| | First Hole | Hole | Third Hole | Driving | Current | Current |
| | Transport | Transport | Transport | Voltage | Efficiency | Efficiency |
| | Layer | Layer | Layer | (V) | (cd/A) | (%) |
| Device | HT1-1 | C-9 | HT3-1 | 2.8 | 33.1 | 100.0 |
| Example 1-1 | | | | | | |
| Device | HT1-1 | C-29 | HT3-1 | 2.9 | 33.7 | 100.0 |
| Example 1-2 | | | | | | |
| Comparative | HT1-1 | HT2-1 | HT3-1 | 3.2 | 32.5 | 98.2 |
| Example 1-1 | | | | | | |
| Comparative | HT1-1 | HT2-2 | HT3-1 | 3.1 | 21.5 | 63.8 |
| Example 1-2 | | | | | | |

### Device Examples 2-1 and 2-2: Producing Red Light-Emitting OLEDs According to the Present Disclosure

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1 was introduced into a cell of the vacuum vapor deposition apparatus, and Compound HT1-2 was introduced into another cell. The two materials were evaporated at different rates, and Compound HI-1 was deposited in a doping amount of 3 wt % based to the total amount of Compound HI-1 and Compound HT1-2 to form a hole injection layer with a thickness of 10 nm. Subsequently, Compound HT1-2 was deposited on the hole injection layer to form a first hole transport layer with a thickness of 90 nm. The compound shown in Table 2 below was then introduced into another cell of the vacuum vapor deposition apparatus and was evaporated by applying an electric current to the cell, to thereby deposit a second hole transport layer with a thickness of 55 nm on the first hole transport layer. Next, Compound HT3-2 was introduced into another cell of the vacuum vapor deposition apparatus and was evaporated by applying an electric current to the cell to thereby deposit a third hole transport layer with a thickness of 5 nm on the second hole transport layer. After formation of the hole injection layer and the hole transport layers, a light-emitting layer was deposited thereon as follows. After Compound H1-2 and Compound H2-1 were introduced into two cells of the vacuum vapor deposition apparatus as hosts, the two compounds were evaporated at a ratio of 1:1. After Compound D-39 was introduced into another cell as a dopant, the dopant was deposited in a doping amount of 2 wt % based on the total amount of the hosts and the dopant to form a light-emitting layer with a thickness of 40 nm on the third hole transport layer. Then an electron buffer layer with a thickness of 5 nm was deposited on the light-emitting layer using Compound B-2 as the electron buffer material. Next, an electron transport layer was deposited to a thickness of 30 nm on the electron buffer layer using a 1:1 weight ratio mixture of Compound ET-2 and Compound EI-1 as the electron transport materials. After Compound EI-1 was deposited as an electron injection layer with a thickness of 2 nm on the electron transport layer, an Al cathode was deposited with a thickness of 80 nm on the electron injection layer using another vacuum vapor deposition apparatus to manufacture an OLED.

### Comparative Example 2-1: Producing Red Light-Emitting OLED not According to the Present Disclosure

An OLED was produced in the same manner as in Device Example 2-1, except that the compound of the second hole transport layer described in Table 2 below was used.

The current efficiency at a luminance of 1,000 nit, and time taken for luminance to reduce from 100% to 95% at a luminance of 10,000 nit (lifetime: T95) of the OLED produced in Device Examples 2-1 and 2-2, and Comparative Example 2-1 as described above were measured, and the results thereof are shown in the following Table 2.

TABLE 2

| | | Second | | | | |
|---|---|---|---|---|---|---|
| | First Hole | Hole | Third Hole | Current | Current | |
| | Transport | Transport | Transport | Efficiency | Efficiency | Lifetime |
| | Layer | Layer | Layer | (cd/A) | (%) | T95 [hr] |
| Device | HT1-2 | C-9 | HT3-2 | 34.1 | 100.0 | 315.5 |
| Example 2-1 | | | | | | |
| Device | HT1-2 | C-29 | HT3-2 | 34.3 | 100.0 | 211.0 |
| Example 2-2 | | | | | | |
| Comparative | HT1-2 | HT2-2 | HT3-2 | 29.8 | 86.9 | 30.9 |
| Example 2-1 | | | | | | |

### Device Examples 3-1 to 3-3: Producing Red Light-Emitting OLEDs According to the Present Disclosure

OLEDs were produced in the same manner as in Device Example 1-1, except that the respective compounds of the second hole transport layer described in Table 3 below were used.

### Comparative Example 3-1: Producing a Red Light-Emitting OLED not According to the Present Disclosure

An OLED was produced in the same manner as in Device Example 3-1, except that the compound of the second hole transport layer described in Table 3 below was used.

The driving voltage, current efficiency, and CIE color coordinates at a luminance of 1,000 nit of the OLED produced in Device Examples 3-1 to 3-3 and Comparative Example 3-1 as described above were measured, and the results thereof are shown in the following Table 3.

TABLE 3

| | | Second | Third | | | | |
|---|---|---|---|---|---|---|---|
| | First Hole | Hole | Hole | Driving | Current | Current | CIE Color |
| | Transport | Transport | Transport | Voltage | Efficiency | Efficiency | Coordinates |
| | Layer | Layer | Layer | (V) | (cd/A) | (%) | (x,y) |
| Device | HT1-1 | C-573 | HT3-1 | 3.0 | 34.0 | 114.8 | 0.661,0.339 |
| Example 3-1 | | | | | | | |
| Device | HT1-1 | C-574 | HT3-1 | 3.0 | 33.0 | 111.4 | 0.659,0.340 |
| Example 3-2 | | | | | | | |
| Device | HT1-1 | C-575 | HT3-1 | 3.0 | 33.6 | 113.5 | 0.659,0.340 |
| Example 3-3 | | | | | | | |
| Comparative | HT1-1 | HT2-3 | HT3-1 | 3.1 | 29.6 | 100 | 0.658,0.341 |
| Example 3-1 | | | | | | | |

### Device Examples 4-1 to 4-3: Producing Red Light-Emitting OLEDs According to the Present Disclosure

OLEDs were produced in the same manner as in Device Example 1-1, except that the respective compounds of the second hole transport layer described in Table 4 below were used, and the respective compounds described in Table 4 below were used as host materials of the light-emitting layer by co-evaporation at a ratio of 4:6.

### Comparative Example 4-1: Producing a Red Light-Emitting OLED not According to the Present Disclosure

An OLED was produced in the same manner as in Device Example 4-1, except that the compound of the second hole transport layer described in Table 4 below was used.

The driving voltage, current efficiency, and CIE color coordinates at a luminance of 1,000 nit of the OLED produced in Device Examples 4-1 to 4-3 and Comparative Example 4-1 as described above were measured, and the results thereof are shown in the following Table 4.

TABLE 4

| | Second | | | | | |
|---|---|---|---|---|---|---|
| | Hole | Light- | Driving | Current | Current | CIE Color |
| | Transport | Emitting | Voltage | Efficiency | Efficiency | Coordinates |
| | Layer | Hosts | (V) | (cd/A) | (%) | (x,y) |
| Device | C-573 | H1-3:H2-2 | 2.9 | 34.5 | 108.9 | 0.661,0.338 |
| Example 4-1 | | | | | | |
| Device | C-574 | H1-3:H2-2 | 3.0 | 34.7 | 109.7 | 0.660,0.339 |
| Example 4-2 | | | | | | |
| Device | C-575 | H1-3:H2-2 | 2.9 | 34.4 | 108.6 | 0.661,0.339 |
| Example 4-3 | | | | | | |
| Comparative | HT2-3 | H1-3:H2-2 | 3.1 | 31.6 | 100 | 0.660,0.340 |
| Example 4-1 | | | | | | |

### Device Examples 5-1 to 5-3: Producing Red Light-Emitting OLEDs According to the Present Disclosure

OLEDs were produced in the same manner as in Device Example 1-1, except that the respective compounds of the second transport layer described in Table 5 below were used, and the respective compounds described in Table 5 below were used as host materials of the light-emitting layer by co-evaporation at a ratio of 4:2:4.

### Comparative Example 5-1: Producing a Red Light-Emitting OLED not According to the Present Disclosure

An OLED was produced in the same manner as in Device Example 5-1, except that the compound of the second transport layer described in Table 5 below was used.

The driving voltage, current efficiency, and CIE color coordinates at a luminance of 1,000 nit of the OLED produced in Device Examples 5-1 to 5-3, and Comparative Examples 5-1 as described above were measured, and the results thereof are shown in the following Table 5.

TABLE 5

| | Second | | | | | |
|---|---|---|---|---|---|---|
| | Hole | Light- | Driving | Current | Current | CIE Color |
| | Transport | Emitting | Voltage | Efficiency | Efficiency | Coordinates |
| | Layer | Hosts | (V) | (cd/A) | (%) | (x,y) |
| Device | C-573 | H1-4:H2-3: | 2.9 | 32.9 | 105.6 | 0.661,0.338 |
| Example 5-1 | | H2-4 | | | | |
| Device | C-574 | H1-4:H2-3: | 3.0 | 33.2 | 106.7 | 0.661,0.339 |
| Example 5-2 | | H2-4 | | | | |
| Device | C-575 | H1-4:H2-3: | 2.9 | 33.2 | 106.5 | 0.661,0.339 |
| Example 5-3 | | H2-4 | | | | |
| Comparative | HT2-3 | H1-4:H2-3: | 3.1 | 31.1 | 100 | 0.660,0.340 |
| Example 5-1 | | H2-4 | | | | |

### Device Examples 6-1 and 6-2: Producing Red Light-Emitting OLEDs According to the Present Disclosure

OLEDs were produced in the same manner as in Device Example 1-1, except that the respective compounds of the second hole transport layer described in Table 6 below were used, and the respective compounds described in Table 6 below were used as host materials of the light-emitting layer by co-evaporation at a ratio of 4:6.

### Comparative Example 6-1: Producing a Red Light-Emitting OLED not According to the Present Disclosure

An OLED was produced in the same manner as in Device Example 6-1, except that the compound of the second hole transport layer described in Table 6 below was used.

The driving voltage, current efficiency, and CIE color coordinates at a luminance of 1,000 nit of the OLED produced in Device Examples 6-1 and 6-2 and Comparative Example 6-1 as described above were measured, and the results thereof are shown in the following Table 6.

TABLE 6

| | Second | | | | |
|---|---|---|---|---|---|
| | Hole | | Current | Current | |
| | Transport | Light-Emitting | Efficiency | Efficiency | CIE Color |
| | Layer | Hosts | (cd/A) | (%) | Coordinates (x, y) |
| Device | C-589 | H1-1: H2-1 | 32.5 | 104 | 0.661, 0.339 |
| Example 6-1 | | | | | |
| Device | C-649 | H1-1: H2-1 | 33.3 | 107 | 0.660, 0.339 |
| Example 6-2 | | | | | |
| Comparative | HT2-4 | H1-1: H2-1 | 31.1 | 100 | 0.661, 0.339 |
| Example 6-1 | | | | | |

From Tables 1 to 6 above, it can be confirmed that the OLEDs comprising the organic electroluminescent compound according to the present disclosure exhibit lower driving voltage, higher current efficiency, and longer lifetime characteristics compared to the organic electroluminescent device using a conventional compound in the second hole transport layer.

The compounds used in the Device Examples and Comparative Examples are shown in Table 7 below.

TABLE 7

| | |
|---|---|
| Hole Injection Layer/Hole Transport Layer | 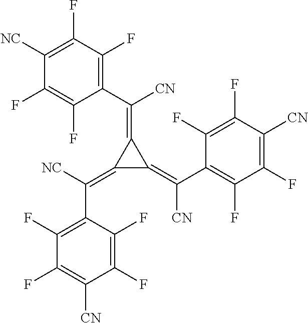 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 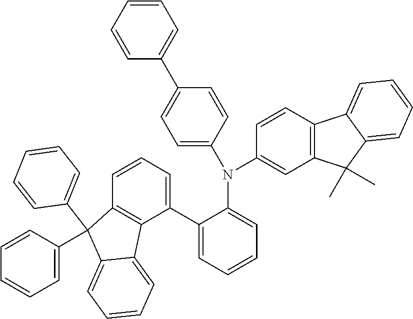 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT1-1 |
| | 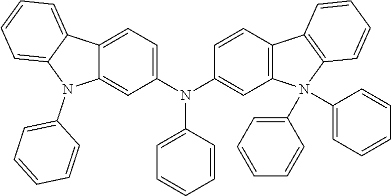 |
| | HT1-2 |
| | 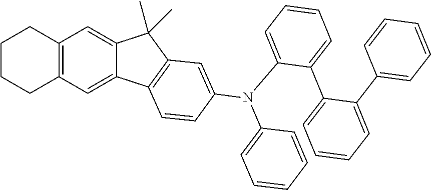 [SMILES: CC1(C)c2cc(N(c3ccccc3)c3ccccc3-c3ccccc3-c3ccccc3)ccc2-c2cc3c(cc21)CCCC3] |
| | C-9 |
| | 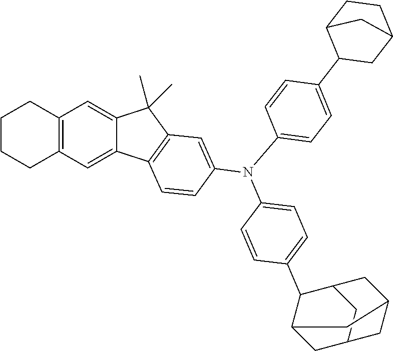 [SMILES: CC1(C)c2cc(N(c3ccc(C4CC5CCC4C5)cc3)c3ccc(C4C5CC6CC(C5)CC4C6)cc3)ccc2-c2cc3c(cc21)CCCC3] |
| | C-29 |
| | 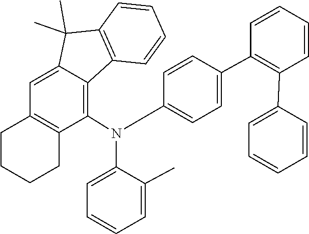 [SMILES: Cc1ccccc1N(c1ccc(-c2ccccc2-c2ccccc2)cc1)c1c2c(cc3c1-c1ccccc1C3(C)C)CCCC2] |
| | C-573 |
| | 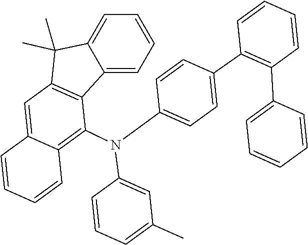 [SMILES: Cc1cccc(N(c2ccc(-c3ccccc3-c3ccccc3)cc2)c2c3c(cc4ccccc24)C(C)(C)c2ccccc2-3)c1] |
| | C-574 |
| | 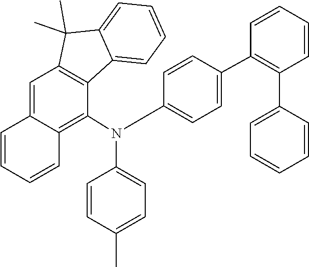 [SMILES: Cc1ccc(N(c2ccc(-c3ccccc3-c3ccccc3)cc2)c2c3c(cc4ccccc24)C(C)(C)c2ccccc2-3)cc1] |
| | C-575 |
| | 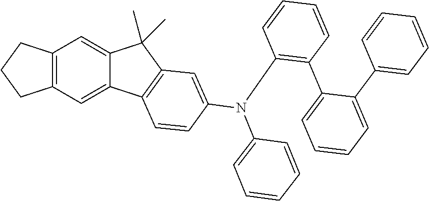 [SMILES: CC1(C)c2cc(N(c3ccccc3)c3ccccc3-c3ccccc3-c3ccccc3)ccc2-c2cc3c(cc21)CCC3] |
| | C-589 |
| | 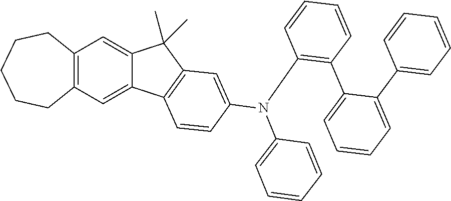 [SMILES: CC1(C)c2cc(N(c3ccccc3)c3ccccc3-c3ccccc3-c3ccccc3)ccc2-c2cc3c(cc21)CCCCC3] |
| | C-649 |
| | 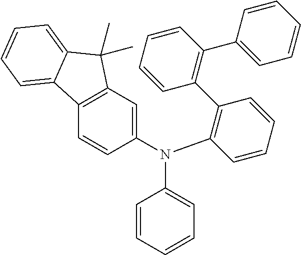 [SMILES: CC1(C)c2ccccc2-c2ccc(N(c3ccccc3)c3ccccc3-c3ccccc3-c3ccccc3)cc21] |
| | HT2-1 |
| | 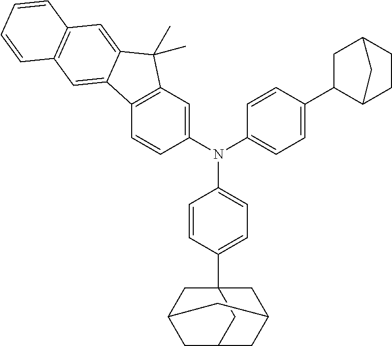 [SMILES: CC1(C)c2cc(N(c3ccc(C4CC5CCC4C5)cc3)c3ccc(C45CC6CC(CC(C6)C4)C5)cc3)ccc2-c2cc3ccccc3cc21] |
| | HT2-2 |
| | 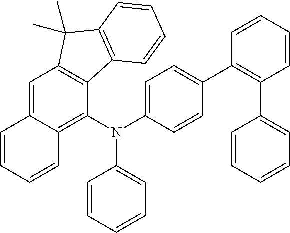 [SMILES: CC1(C)c2ccccc2-c2c1cc1ccccc1c2N(c1ccccc1)c1ccc(-c2ccccc2-c2ccccc2)cc1] |
| | HT2-3 |
| | 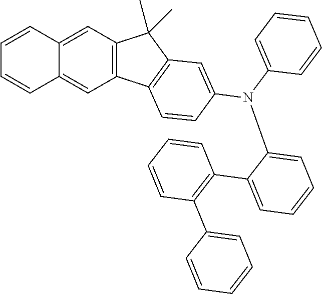 [SMILES: CC1(C)c2cc(N(c3ccccc3)c3ccccc3-c3ccccc3-c3ccccc3)ccc2-c2cc3ccccc3cc21] |
| | HT2-4 |
| | 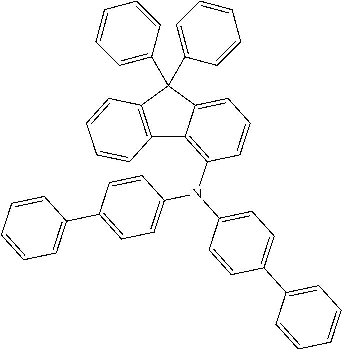 [SMILES: c1ccc(-c2ccc(N(c3ccc(-c4ccccc4)cc3)c3cccc4c3-c3ccccc3C4(c3ccccc3)c3ccccc3)cc2)cc1] |
| | HT3-1 |
| | 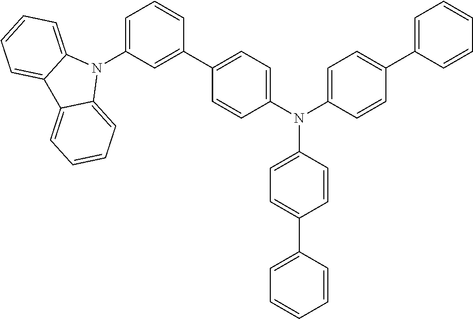 [SMILES: c1ccc(-c2ccc(N(c3ccc(-c4ccccc4)cc3)c3ccc(-c4cccc(-n5c6ccccc6c6ccccc65)c4)cc3)cc2)cc1] |
| | HT3-2 |
| Light-Emitting Layer | 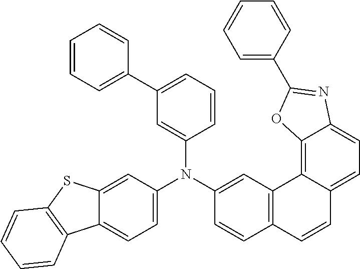 [SMILES: c1ccc(-c2cccc(N(c3ccc4c(c3)sc3ccccc34)c3ccc4ccc5ccc6nc(-c7ccccc7)oc6c5c4c3)c2)cc1] |
| | H1-1 |
| | 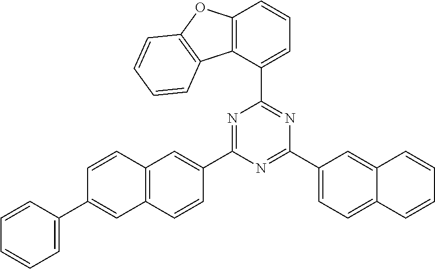 [SMILES: c1ccc(-c2ccc3cc(-c4nc(-c5ccc6ccccc6c5)nc(-c5cccc6oc7ccccc7c56)n4)ccc3c2)cc1] |
| | H2-1 |
| | 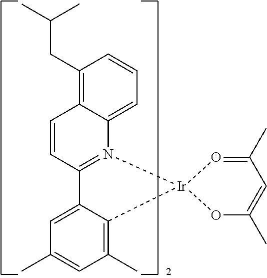 |
| | D-39 |
| | 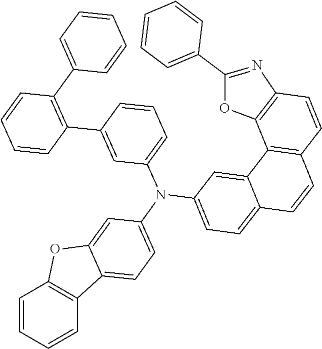 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | H1-2 |
| | 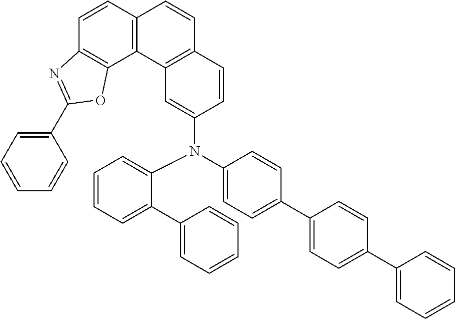 [SMILES: c1ccc(-c2ccc(-c3ccc(N(c4ccc5ccc6ccc7nc(-c8ccccc8)oc7c6c5c4)c4ccccc4-c4ccccc4)cc3)cc2)cc1] |
| | H1-3 |
| | 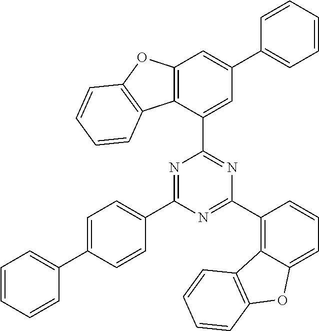 [SMILES: c1ccc(-c2ccc(-c3nc(-c4cccc5oc6ccccc6c45)nc(-c4cc(-c5ccccc5)cc5oc6ccccc6c45)n3)cc2)cc1] |
| | H2-2 |
| | 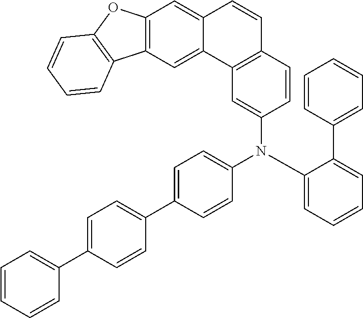 [SMILES: c1ccc(-c2ccc(-c3ccc(N(c4ccc5ccc6cc7oc8ccccc8c7cc6c5c4)c4ccccc4-c4ccccc4)cc3)cc2)cc1] |
| | H1-4 |
| | 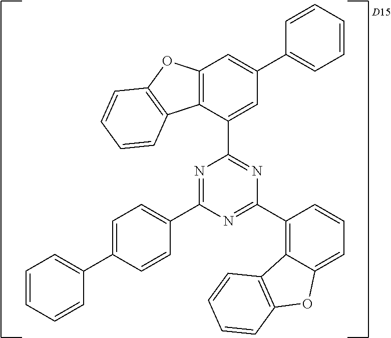 |
| | H2-3 |
| | 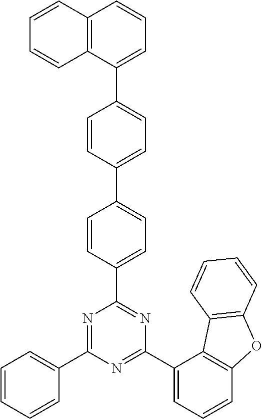 [SMILES: c1ccc(-c2nc(-c3ccc(-c4ccc(-c5cccc6ccccc56)cc4)cc3)nc(-c3cccc4oc5ccccc5c34)n2)cc1] |
| | H2-4 |
| Electron Buffer Layer/Electron Transport Layer/ Electron Injection Layer | 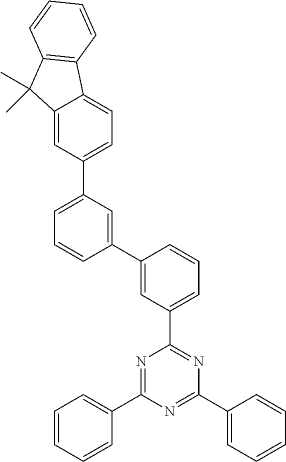 [SMILES: CC1(C)c2ccccc2-c2ccc(-c3cccc(-c4cccc(-c5nc(-c6ccccc6)nc(-c6ccccc6)n5)c4)c3)cc21] |
| | B-1 |
| | 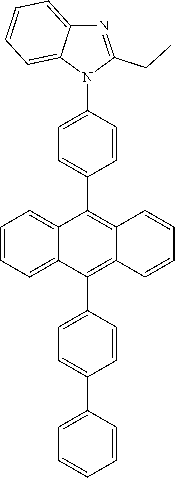 [SMILES: CCc1nc2ccccc2n1-c1ccc(-c2c3ccccc3c(-c3ccc(-c4ccccc4)cc3)c3ccccc23)cc1] |
| | ET-1 |
| | 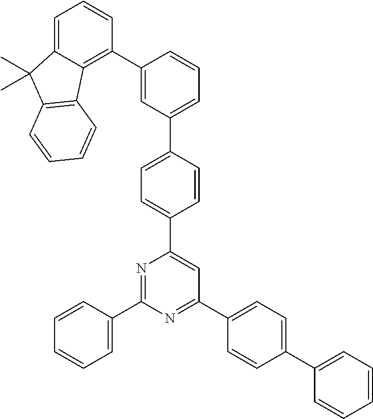 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | B-2 |
| | 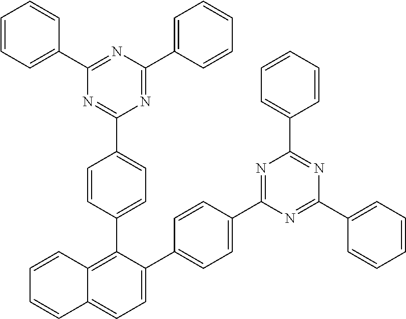 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1] |
| | ET-2 |
| | 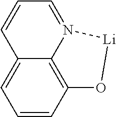 |
| | EI-1 |

1. An organic electroluminescent compound represented by the following Formula 1: 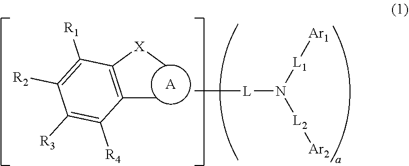
In Formula 1,
X represents —O—, —S—, —Se—, —Ge— or —(CR₉R₁₀)ₙ—;
ring A represents a substituted or unsubstituted benzene ring, or a substituted or unsubstituted naphthalene ring, and two adjacent carbon atoms in ring A are each bonded to the * positions in Formula 1-A to form an aliphatic ring; 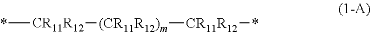
R₁ to R₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L-N(L₁-Ar₁)(L₂-Ar₂);
R₉ and R₁₀ each independently represent a substituted or unsubstituted (C1-C30)alkyl, or a substituted or unsubstituted (C6-C30)aryl, or R₉ and R₁₀ may be linked to each other to form a ring(s);
R₁₁ and R₁₂ each independently represent hydrogen, deuterium, a halogen, a substituted or unsubstituted (C1-C30)alkyl, or a substituted or unsubstituted (C6-C30)aryl, and each R₁ or each R₁₂ may be the same as or different from each other;
L, L₁, and L₂ each independently represent a single bond, a substituted or unsubstituted (C6-C30)arylene, a substituted or unsubstituted (3- to 30-membered)heteroarylene, or a substituted or unsubstituted (C3-C30)cycloalkylene;
L is linked to at least one selected from the group consisting of a carbon atom bonded to any one of R₁ to R₄, and a carbon atom of the benzene ring or the naphthalene ring of the ring A;
Ar₁ and Ar₂ each independently represent a substituted or unsubstituted (C6-C30)aryl, a substituted or unsubstituted (3- to 30-membered)heteroaryl, or a substituted or unsubstituted (C3-C30)cycloalkyl;
n represents an integer of 1 or 2, m represents an integer of 1 to 3, and a represents an integer of 1 or 2;
hydrogen atoms in the compound represented by Formula 1 may be substituted with deuterium; and
with the proviso that if ring A is a substituted or unsubstituted benzene ring, L₁, L₂, Ar₁, and Ar₂ do not comprise a heteroaryl or an aryl fused with a cycloalkyl.

2. The organic electroluminescent compound according to claim 1, wherein the substituted alkyl, the substituted aryl(ene), the substituted heteroaryl(ene), the substituted cycloalkyl(ene), the substituted alkoxy, the substituted trialkylsilyl, the substituted dialkylarylsilyl, the substituted alkyldiarylsilyl, the substituted triarylsilyl, the fused ring group of the substituted aliphatic ring(s) and aromatic ring(s), the substituted benzene ring, and the substituted naphthalene ring each independently are substituted with at least one selected from the group consisting of deuterium, a halogen, a cyano, a carboxyl, a nitro, a hydroxy, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (3- to 30-membered)heteroaryl, a (C6-C30)aryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, a fused ring group of a (C3-C30) aliphatic ring(s) and a (C6-C30) aromatic ring(s), an amino, a mono- or di(C1-C30)alkylamino, a mono- or di(C6-C30)arylamino, a (C1-C30)alkyl(C6-C30)arylamino, a mono- or di(3- to 30-membered)heteroarylamino, a (C1-C30)alkyl(3- to 30-membered)heteroarylamino, a (C6-C30)aryl(3- to 30-membered)heteroarylamino, a (C1-C30)alkylcarbonyl, a (C1-C30)alkoxycarbonyl, a (C6-C30)arylcarbonyl, a (C6-C30)arylphosphinyl, a di(C6-C30)arylboronyl, a di(C1-C30)alkylboronyl, a (C1-C30)alkyl(C6-C30)arylboronyl, a (C6-C30)aryl(C1-C30)alkyl, a (C1-C30)alkyl(C6-C30)aryl, and combinations thereof.

3. The organic electroluminescent compound according to claim 1, wherein Formula 1 is represented by any one of the following Formulas 2-1 to 2-4: 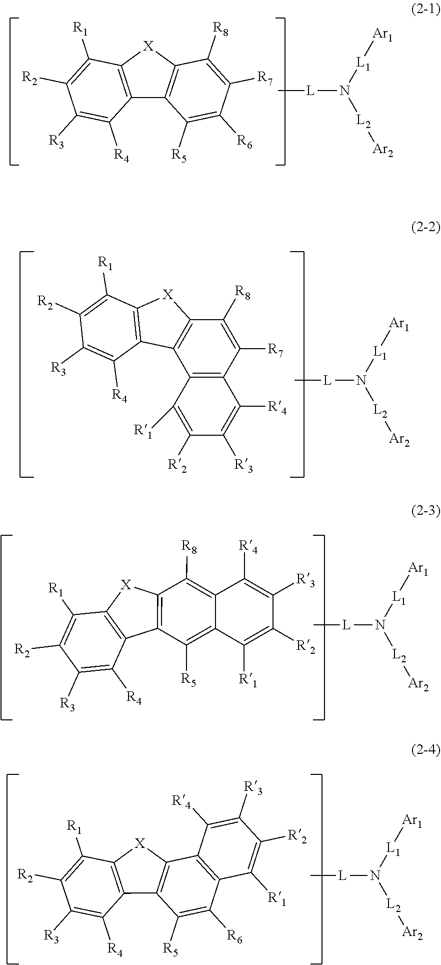
where in Formulas 2-1 to 2-4,
one pair selected from R₅ and Re, R₆ and R₇, and R₇ and R₈ in Formula 2-1 is each linked to * positions of Formula 1-A;
one pair selected from R′₁ and R′₂, R′₂ and R′₃, and R′₃ and R′₄ in Formulas 2-2 to 2-4 is each linked to * positions of Formula 1-A;
X, R₁ to R₄, L, L₁, L₂, Ar₁, and Ar₂ are as defined in claim 1; and
R₅ to R₈ and R′₁ to R′₄ which are not linked to Formula 1-A are each independently the same as defined for R₁.

4. The organic electroluminescent compound according to claim 1, wherein Formula 1 is represented by any one of the following Formulas 3-1 to 3-4: 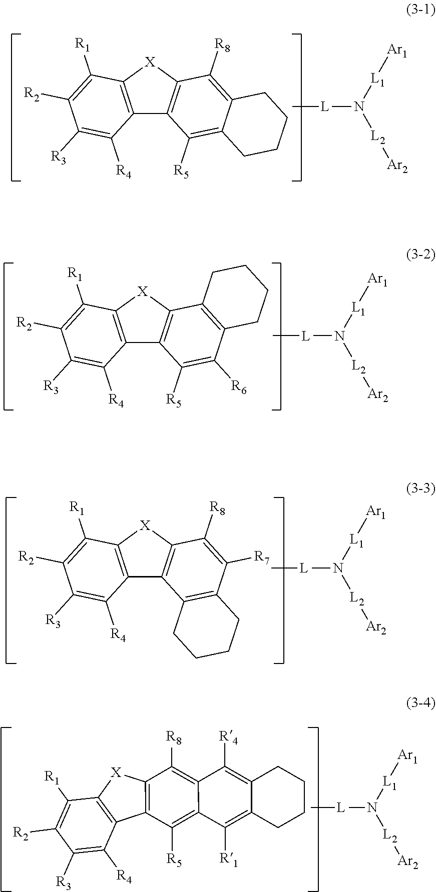
wherein in Formulas 3-1 to 3-4,
X, R₁ to R₄, L, L₁, L₂, Ar₁, and Ar₂ are as defined in claim 1; and
R₅ to R₈, R′₁ and R′₄ are each independently the same as defined for R₁.

5. The organic electroluminescent compound according to claim 1, wherein Formula 1 is represented by any one of the following Formulas 4-1 to 4-4: 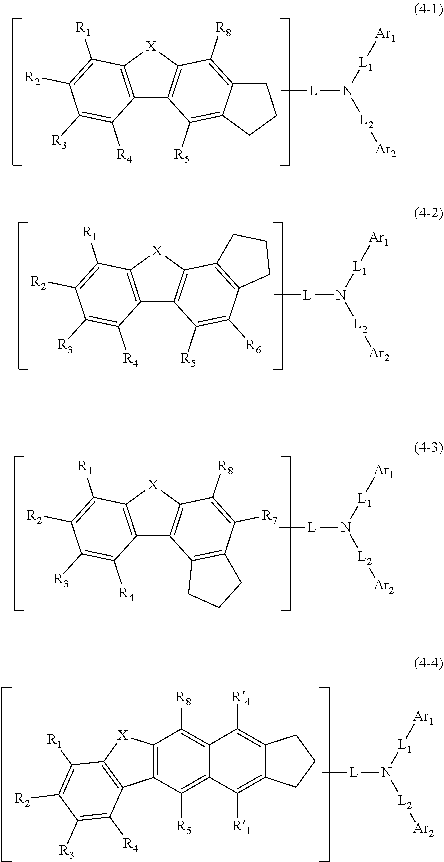
wherein in Formulas 4-1 to 4-4,
X, R₁ to R₄, L, L₁, L₂, Ar₁, and Ar₂ are as defined in claim 1; and
R₅ to R₈, R′₁ and R′₄ are each independently the same as defined for R₁.

6. The organic electroluminescent compound according to claim 1, wherein Formula 1 is represented by any one of the following Formulas 5-1 to 5-4: 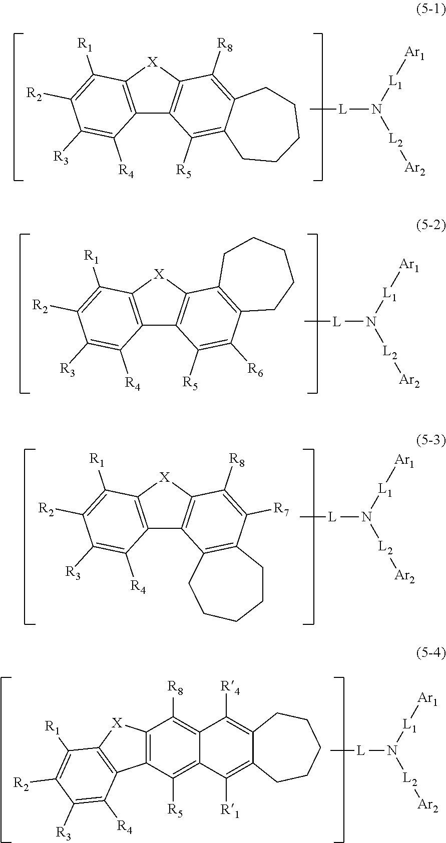
wherein in Formulas 5-1 to 5-4,
X, R₁ to R₄, L, L₁, L₂, Ar₁, and Ar₂ are as defined in claim 1; and
R₅ to R₈, R′₁ and R′₄ are each independently the same as defined for R₁.

7. The organic electroluminescent compound according to claim 1, wherein at least one of Ar₁ and Ar₂ comprises a (C6-C30)aryl substituted with a (C1-C30)alkyl or a (C3-C30)cycloalkyl.

8. The organic electroluminescent compound according to claim 1, wherein the compound represented by Formula 1 is selected from the following compounds:

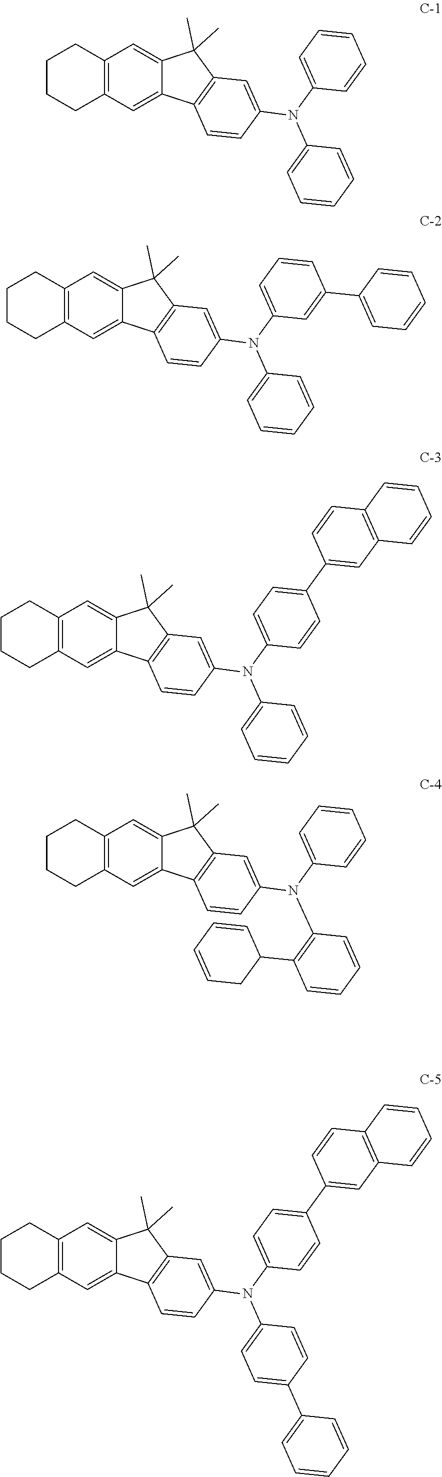 [SMILES: CC1(C)c2cc(N(c3ccccc3)c3ccccc3)ccc2-c2cc3c(cc21)CCCC3 ; CC1(C)c2cc(N(c3ccccc3)c3cccc(-c4ccccc4)c3)ccc2-c2cc3c(cc21)CCCC3 ; CC1(C)c2cc(N(c3ccccc3)c3ccc(-c4ccc5ccccc5c4)cc3)ccc2-c2cc3c(cc21)CCCC3 ; CC1(C)c2cc(N(c3ccccc3)c3ccccc3C3C=CC=CC3)ccc2-c2cc3c(cc21)CCCC3 ; CC1(C)c2cc(N(c3ccc(-c4ccccc4)cc3)c3ccc(-c4ccc5ccccc5c4)cc3)ccc2-c2cc3c(cc21)CCCC3]

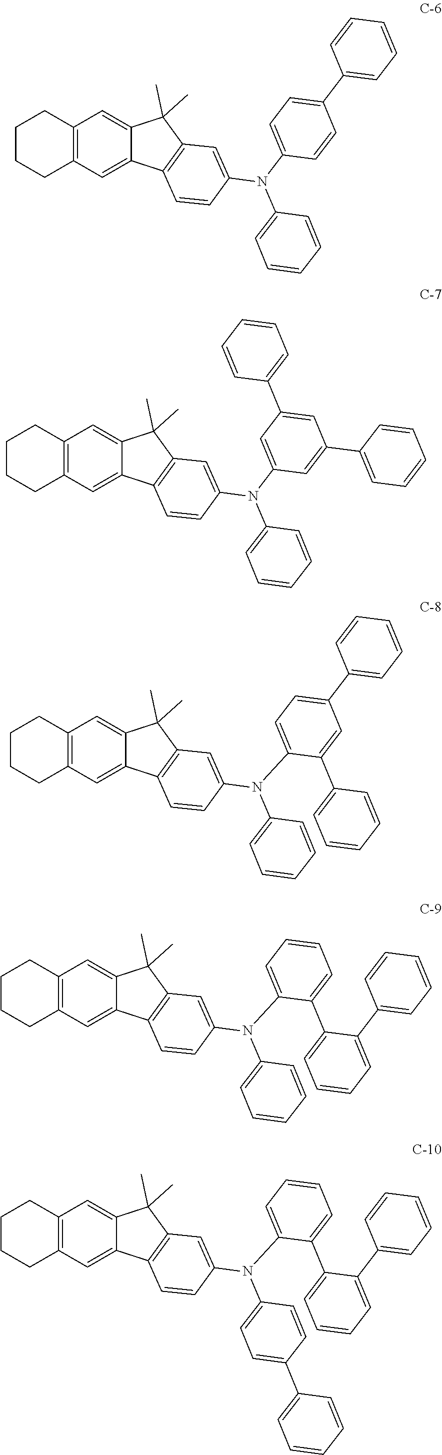 [SMILES: CC1(C)c2cc(N(c3ccccc3)c3ccc(-c4ccccc4)cc3)ccc2-c2cc3c(cc21)CCCC3 ; CC1(C)c2cc(N(c3ccccc3)c3cc(-c4ccccc4)cc(-c4ccccc4)c3)ccc2-c2cc3c(cc21)CCCC3 ; CC1(C)c2cc(N(c3ccccc3)c3ccc(-c4ccccc4)cc3-c3ccccc3)ccc2-c2cc3c(cc21)CCCC3 ; CC1(C)c2cc(N(c3ccccc3)c3ccccc3-c3ccccc3-c3ccccc3)ccc2-c2cc3c(cc21)CCCC3 ; CC1(C)c2cc(N(c3ccc(-c4ccccc4)cc3)c3ccccc3-c3ccccc3-c3ccccc3)ccc2-c2cc3c(cc21)CCCC3]

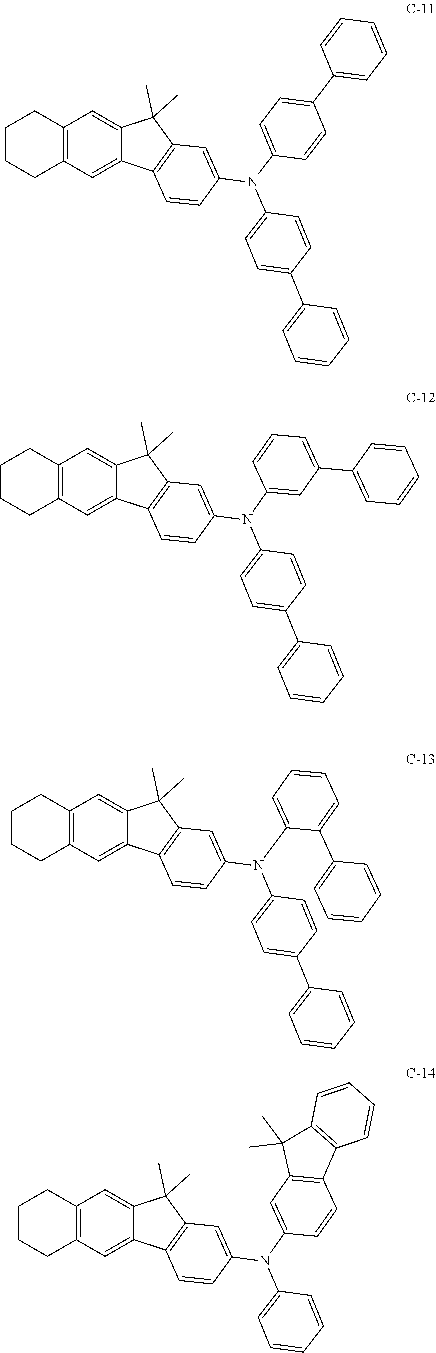

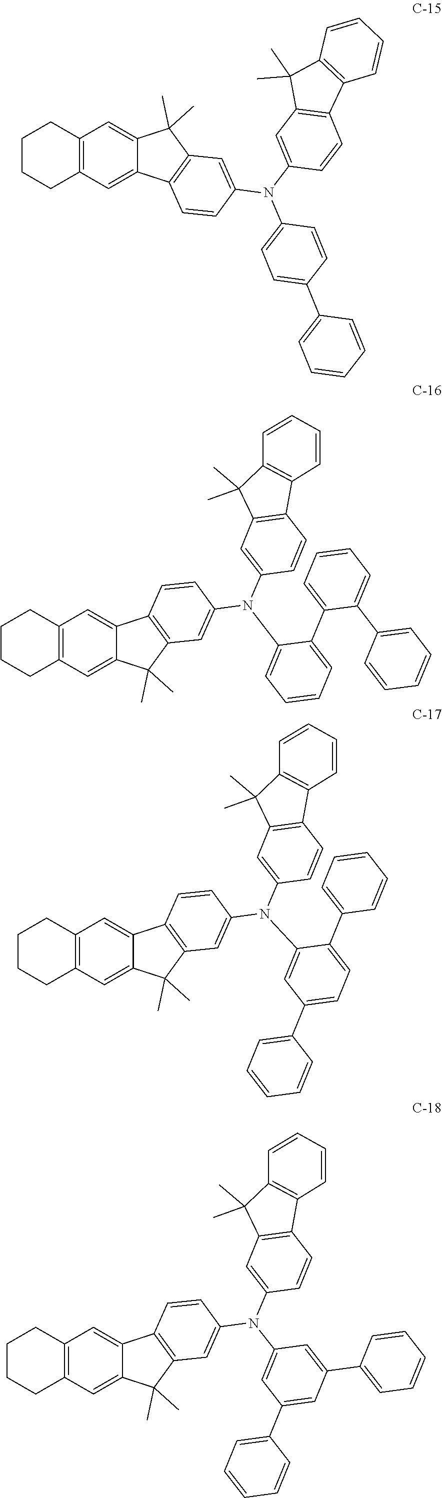

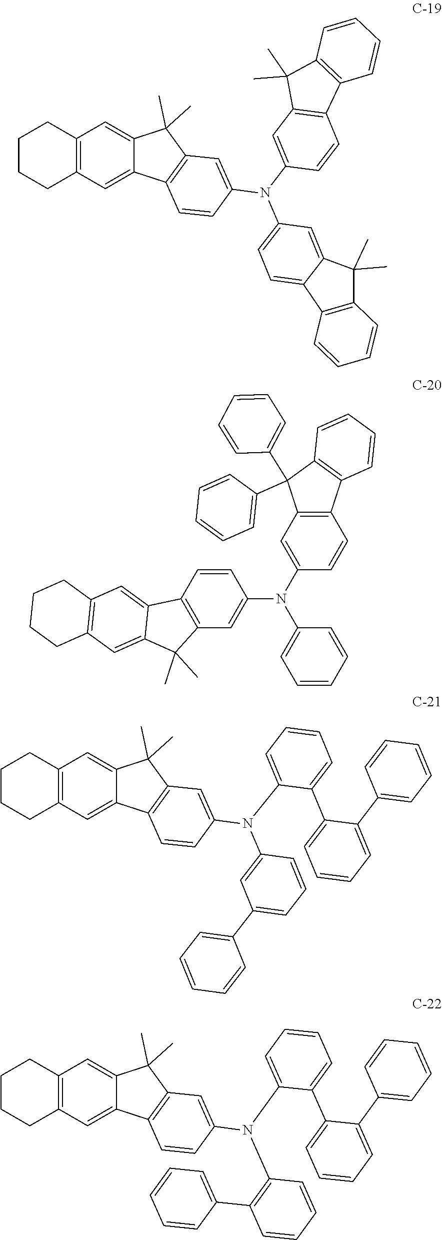

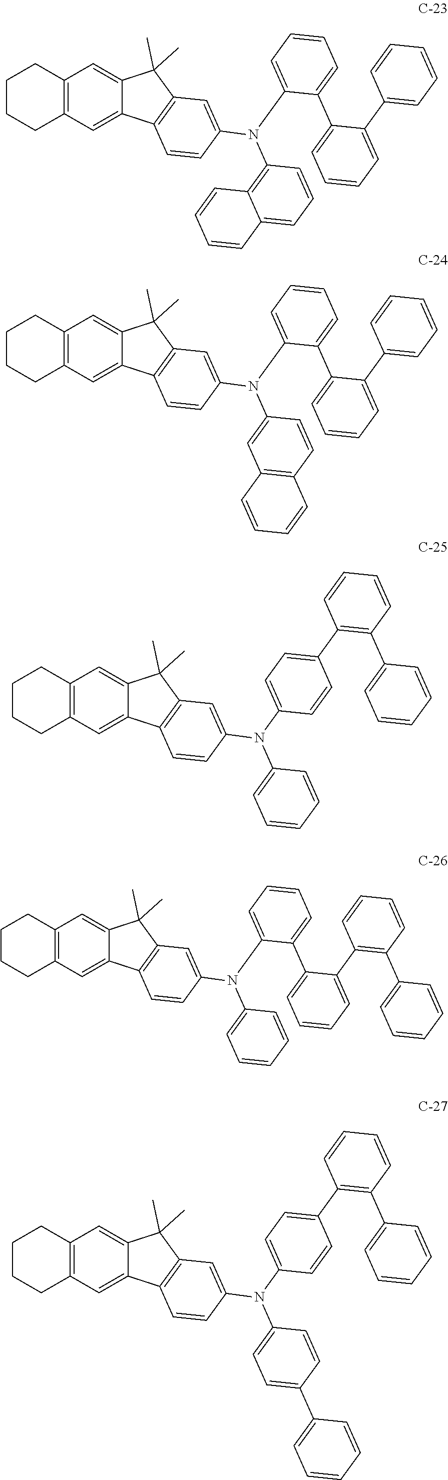 [SMILES: CC1(C)c2cc(N(c3ccccc3-c3ccccc3-c3ccccc3)c3cccc4ccccc34)ccc2-c2cc3c(cc21)CCCC3 ; CC1(C)c2cc(N(c3ccc4ccccc4c3)c3ccccc3-c3ccccc3-c3ccccc3)ccc2-c2cc3c(cc21)CCCC3 ; CC1(C)c2cc(N(c3ccccc3)c3ccc(-c4ccccc4-c4ccccc4)cc3)ccc2-c2cc3c(cc21)CCCC3 ; CC1(C)c2cc(N(c3ccccc3)c3ccccc3-c3ccccc3-c3ccccc3-c3ccccc3)ccc2-c2cc3c(cc21)CCCC3 ; CC1(C)c2cc(N(c3ccc(-c4ccccc4)cc3)c3ccc(-c4ccccc4-c4ccccc4)cc3)ccc2-c2cc3c(cc21)CCCC3]

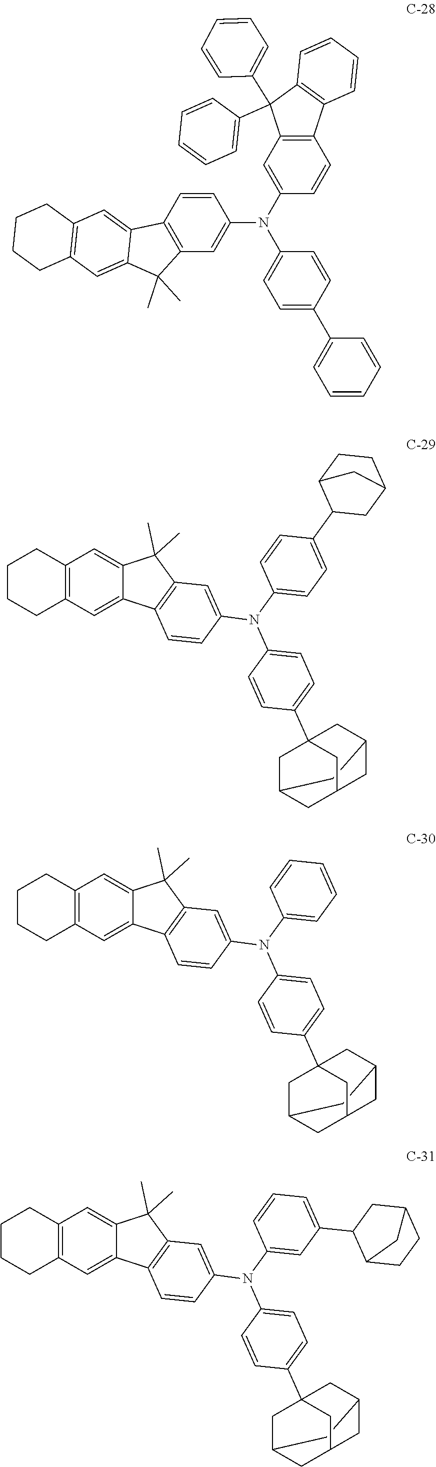 [SMILES: CC1(C)c2cc(N(c3ccc(-c4ccccc4)cc3)c3ccc4c(c3)C(c3ccccc3)(c3ccccc3)c3ccccc3-4)ccc2-c2cc3c(cc21)CCCC3 ; CC1(C)c2cc(N(c3ccc(C4CC5CCC4C5)cc3)c3ccc(C45CC6CC(CC(C6)C4)C5)cc3)ccc2-c2cc3c(cc21)CCCC3 ; CC1(C)c2cc(N(c3ccccc3)c3ccc(C45CC6CC(CC(C6)C4)C5)cc3)ccc2-c2cc3c(cc21)CCCC3 ; CC1(C)c2cc(N(c3ccc(C45CC6CC(CC(C6)C4)C5)cc3)c3cccc(C4CC5CCC4C5)c3)ccc2-c2cc3c(cc21)CCCC3]

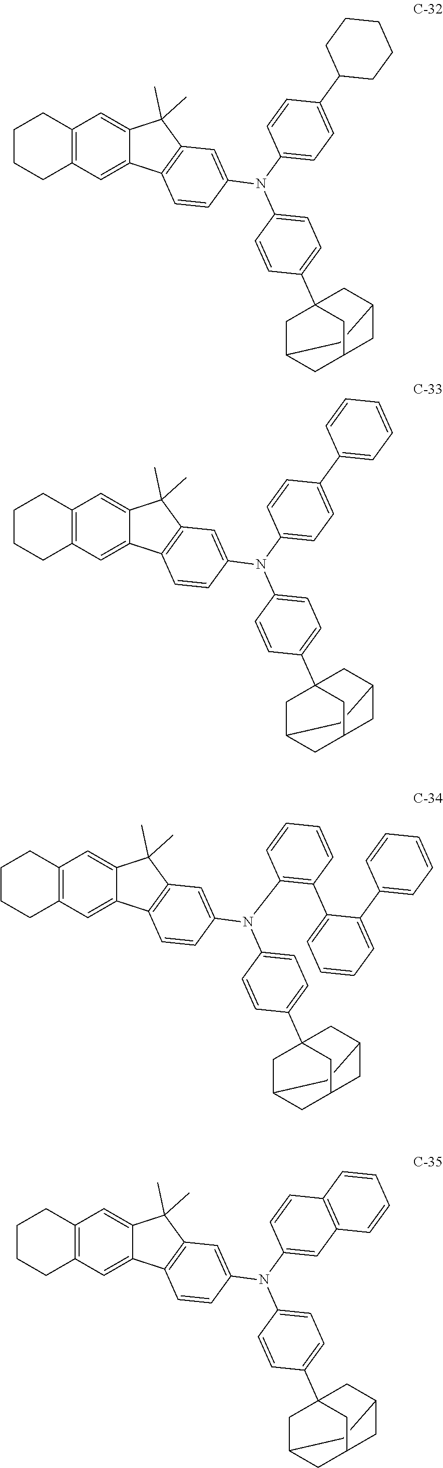

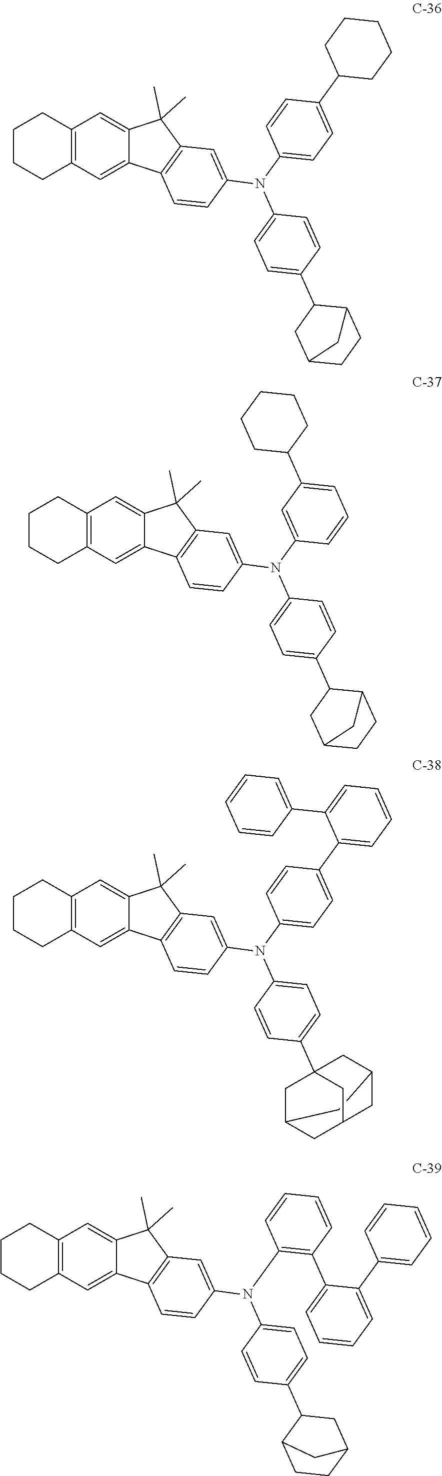

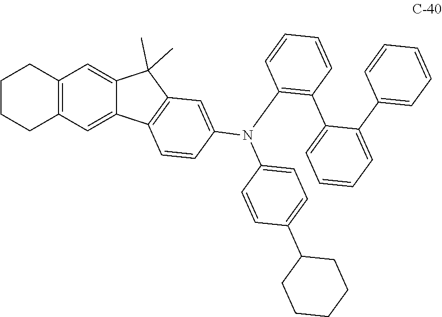 [SMILES: CC1(C)c2cc(N(c3ccc(C4CCCCC4)cc3)c3ccccc3-c3ccccc3-c3ccccc3)ccc2-c2cc3c(cc21)CCCC3]

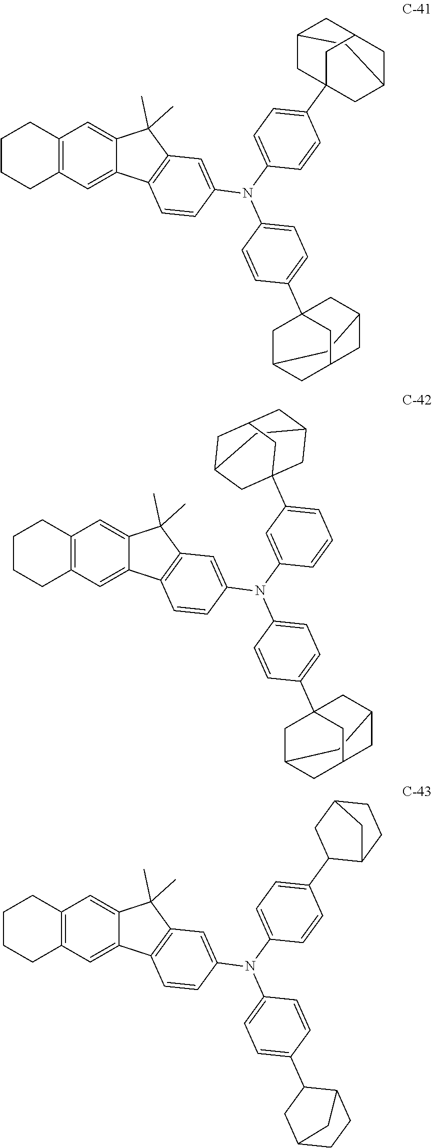

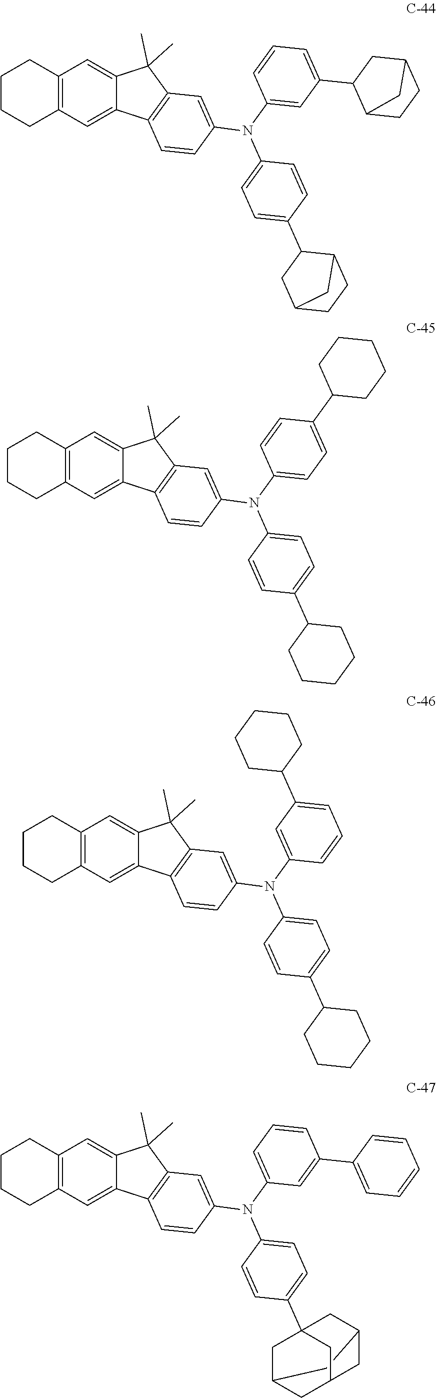

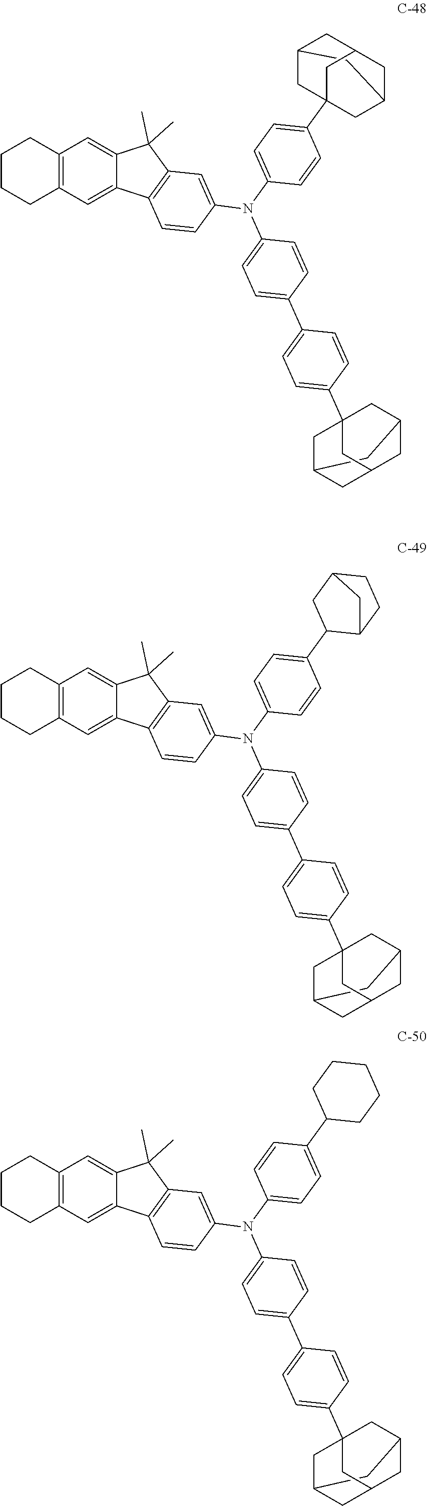

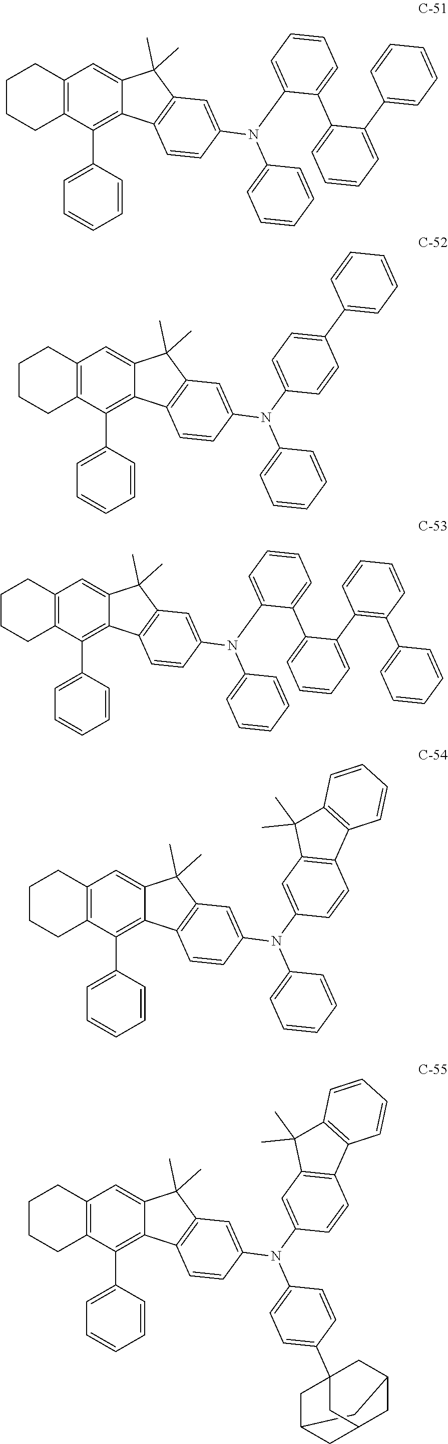

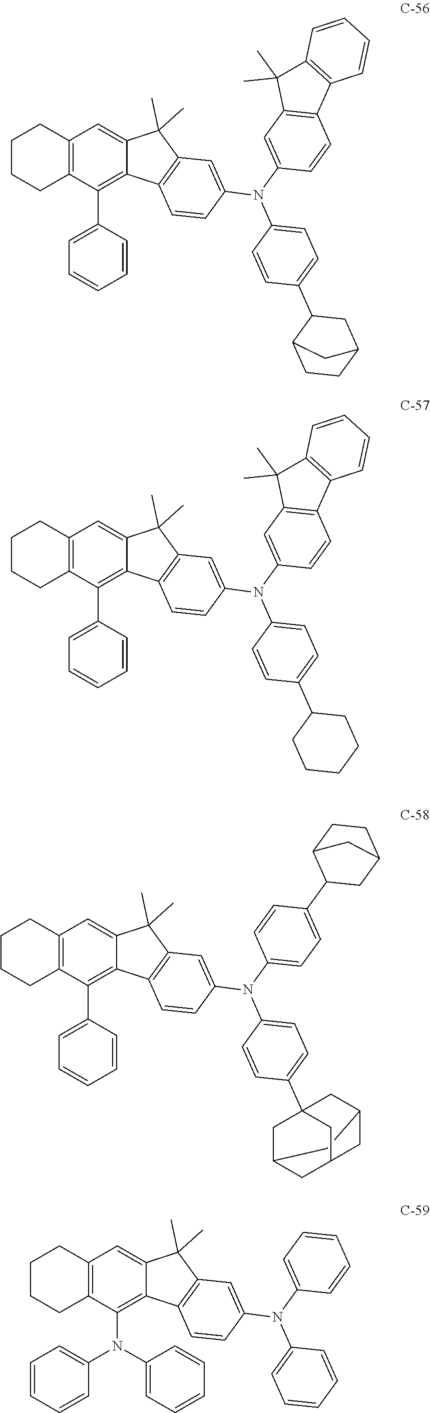

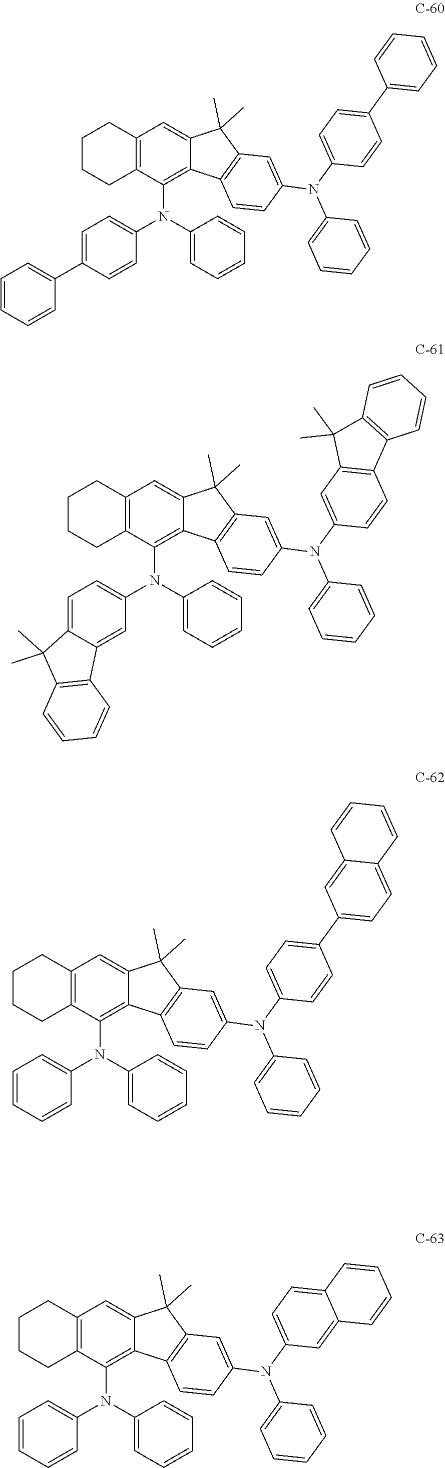 [SMILES: CC1(C)c2cc(N(c3ccccc3)c3ccc(-c4ccccc4)cc3)ccc2-c2c1cc1c(c2N(c2ccccc2)c2ccc(-c3ccccc3)cc2)CCCC1 ; CC1(C)c2ccccc2-c2cc(N(c3ccccc3)c3c4c(cc5c3-c3ccc(N(c6ccccc6)c6ccc7c(c6)C(C)(C)c6ccccc6-7)cc3C5(C)C)CCCC4)ccc21 ; CC1(C)c2cc(N(c3ccccc3)c3ccc(-c4ccc5ccccc5c4)cc3)ccc2-c2c1cc1c(c2N(c2ccccc2)c2ccccc2)CCCC1 ; CC1(C)c2cc(N(c3ccccc3)c3ccc4ccccc4c3)ccc2-c2c1cc1c(c2N(c2ccccc2)c2ccccc2)CCCC1]

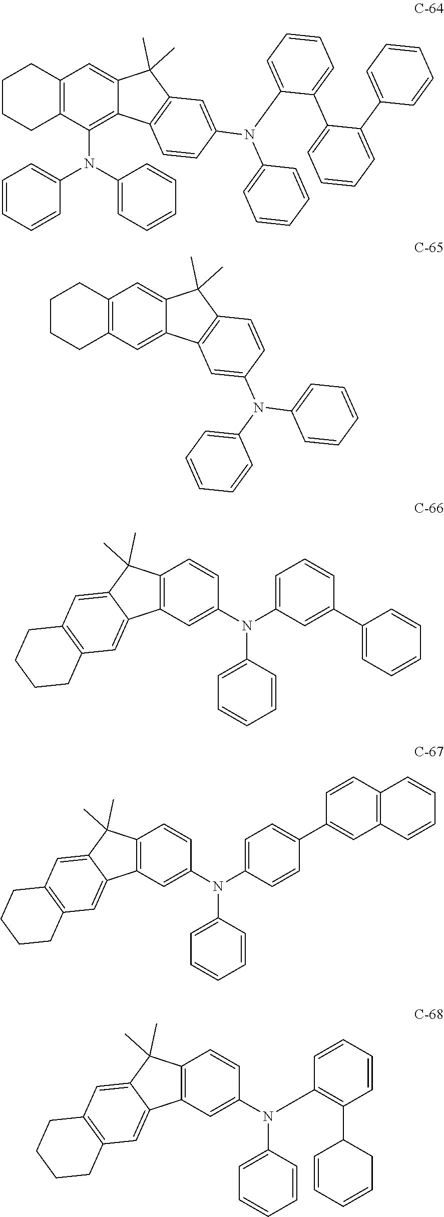

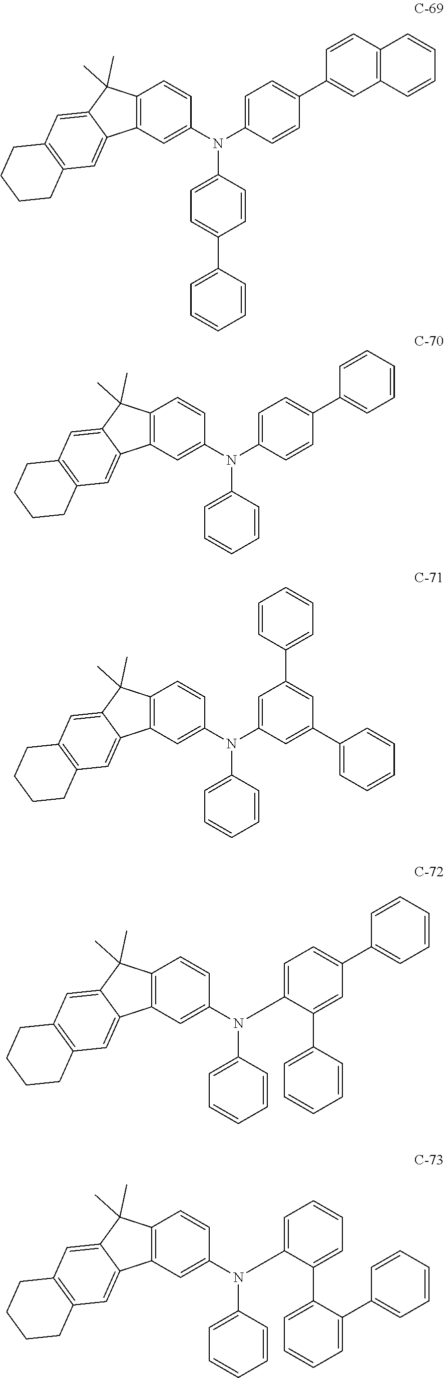 [SMILES: CC1(C)c2ccc(N(c3ccc(-c4ccccc4)cc3)c3ccc(-c4ccc5ccccc5c4)cc3)cc2-c2cc3c(cc21)CCCC3 ; CC1(C)c2ccc(N(c3ccccc3)c3ccc(-c4ccccc4)cc3)cc2-c2cc3c(cc21)CCCC3 ; CC1(C)c2ccc(N(c3ccccc3)c3cc(-c4ccccc4)cc(-c4ccccc4)c3)cc2-c2cc3c(cc21)CCCC3 ; CC1(C)c2ccc(N(c3ccccc3)c3ccc(-c4ccccc4)cc3-c3ccccc3)cc2-c2cc3c(cc21)CCCC3 ; CC1(C)c2ccc(N(c3ccccc3)c3ccccc3-c3ccccc3-c3ccccc3)cc2-c2cc3c(cc21)CCCC3]

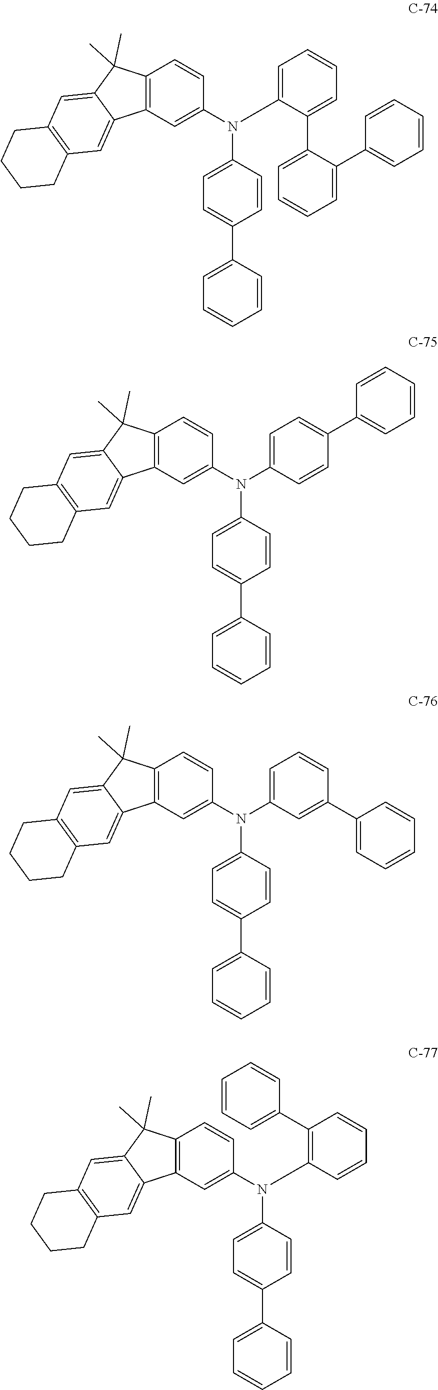

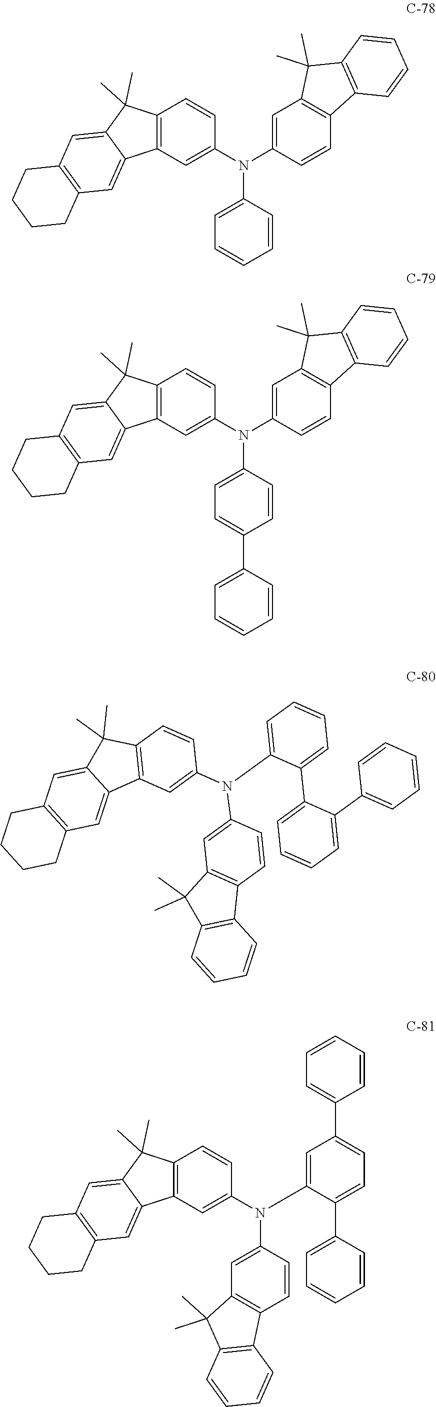 [SMILES: CC1(C)c2ccccc2-c2ccc(N(c3ccccc3)c3ccc4c(c3)-c3cc5c(cc3C4(C)C)CCCC5)cc21 ; CC1(C)c2ccccc2-c2ccc(N(c3ccc(-c4ccccc4)cc3)c3ccc4c(c3)-c3cc5c(cc3C4(C)C)CCCC5)cc21 ; CC1(C)c2ccccc2-c2ccc(N(c3ccc4c(c3)-c3cc5c(cc3C4(C)C)CCCC5)c3ccccc3-c3ccccc3-c3ccccc3)cc21 ; CC1(C)c2ccccc2-c2ccc(N(c3ccc4c(c3)-c3cc5c(cc3C4(C)C)CCCC5)c3cc(-c4ccccc4)ccc3-c3ccccc3)cc21]

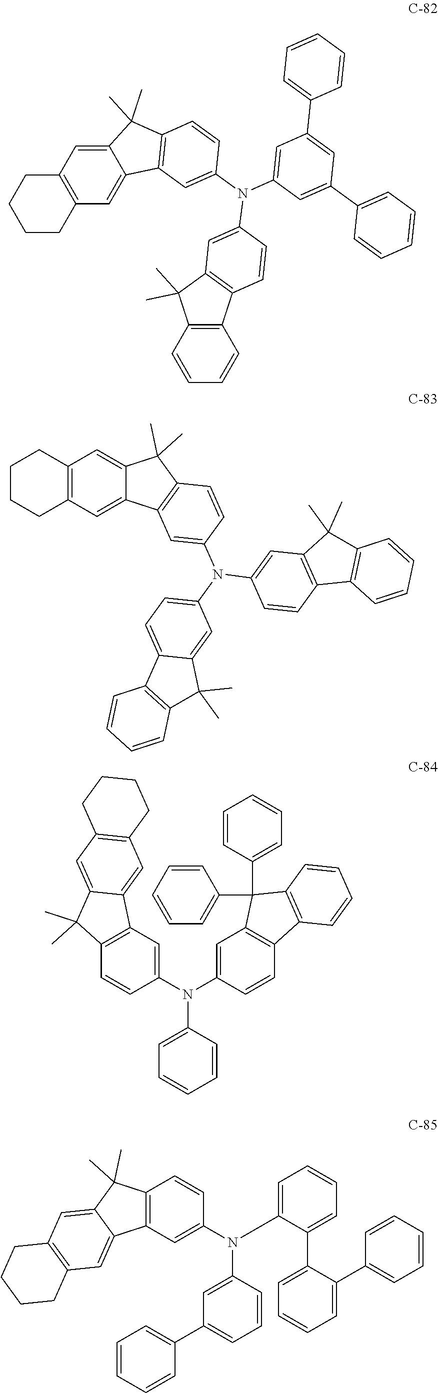

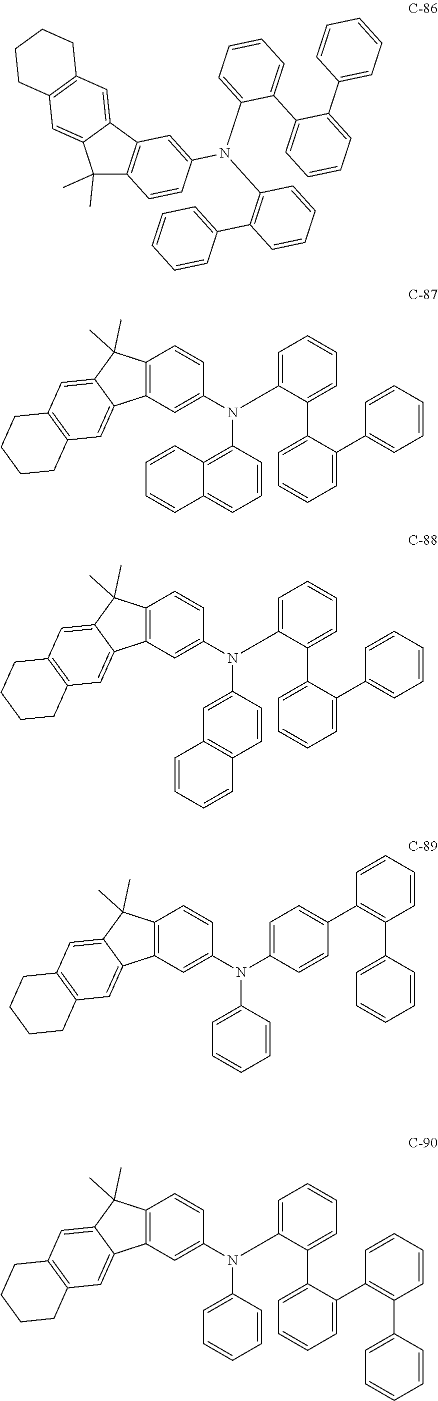 [SMILES: CC1(C)c2ccc(N(c3ccccc3-c3ccccc3)c3ccccc3-c3ccccc3-c3ccccc3)cc2-c2cc3c(cc21)CCCC3 ; CC1(C)c2ccc(N(c3ccccc3-c3ccccc3-c3ccccc3)c3cccc4ccccc34)cc2-c2cc3c(cc21)CCCC3 ; CC1(C)c2ccc(N(c3ccc4ccccc4c3)c3ccccc3-c3ccccc3-c3ccccc3)cc2-c2cc3c(cc21)CCCC3 ; CC1(C)c2ccc(N(c3ccccc3)c3ccc(-c4ccccc4-c4ccccc4)cc3)cc2-c2cc3c(cc21)CCCC3 ; CC1(C)c2ccc(N(c3ccccc3)c3ccccc3-c3ccccc3-c3ccccc3-c3ccccc3)cc2-c2cc3c(cc21)CCCC3]

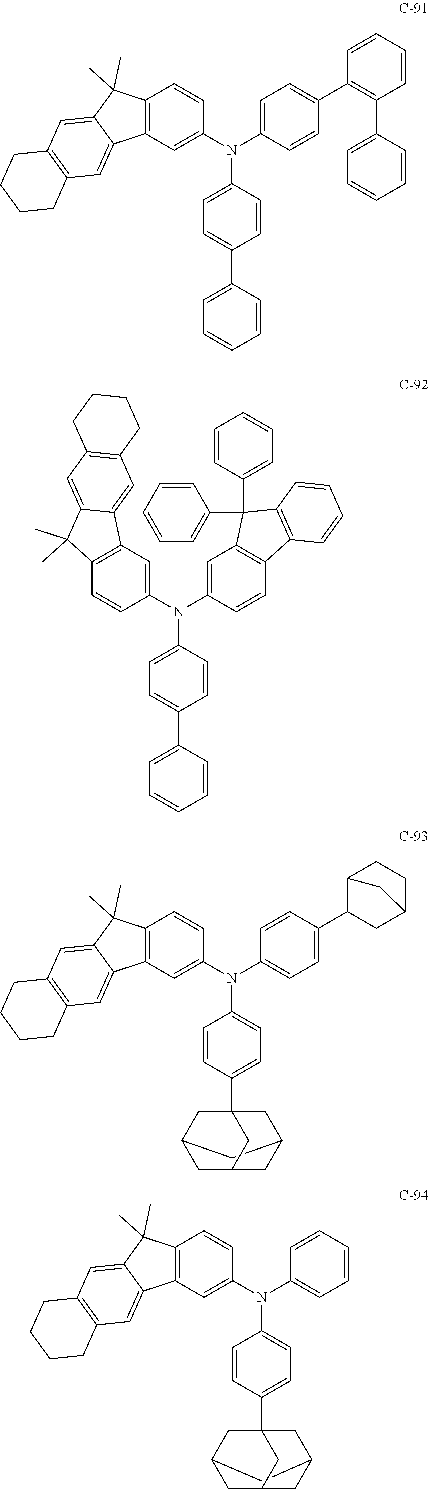 [SMILES: CC1(C)c2ccc(N(c3ccc(-c4ccccc4)cc3)c3ccc(-c4ccccc4-c4ccccc4)cc3)cc2-c2cc3c(cc21)CCCC3 ; CC1(C)c2ccc(N(c3ccc(-c4ccccc4)cc3)c3ccc4c(c3)C(c3ccccc3)(c3ccccc3)c3ccccc3-4)cc2-c2cc3c(cc21)CCCC3 ; CC1(C)c2ccc(N(c3ccc(C4CC5CCC4C5)cc3)c3ccc(C45CC6CC(CC(C6)C4)C5)cc3)cc2-c2cc3c(cc21)CCCC3 ; CC1(C)c2ccc(N(c3ccccc3)c3ccc(C45CC6CC(CC(C6)C4)C5)cc3)cc2-c2cc3c(cc21)CCCC3]

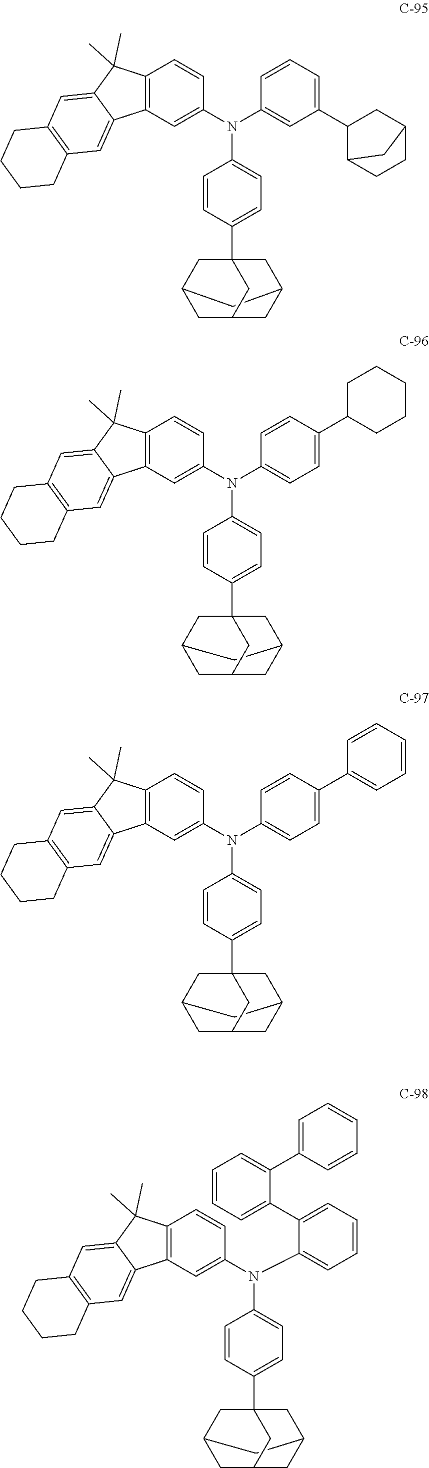 [SMILES: CC1(C)c2ccc(N(c3ccc(C45CC6CC(CC(C6)C4)C5)cc3)c3cccc(C4CC5CCC4C5)c3)cc2-c2cc3c(cc21)CCCC3 ; CC1(C)c2ccc(N(c3ccc(C4CCCCC4)cc3)c3ccc(C45CC6CC(CC(C6)C4)C5)cc3)cc2-c2cc3c(cc21)CCCC3 ; CC1(C)c2ccc(N(c3ccc(-c4ccccc4)cc3)c3ccc(C45CC6CC(CC(C6)C4)C5)cc3)cc2-c2cc3c(cc21)CCCC3 ; CC1(C)c2ccc(N(c3ccc(C45CC6CC(CC(C6)C4)C5)cc3)c3ccccc3-c3ccccc3-c3ccccc3)cc2-c2cc3c(cc21)CCCC3]

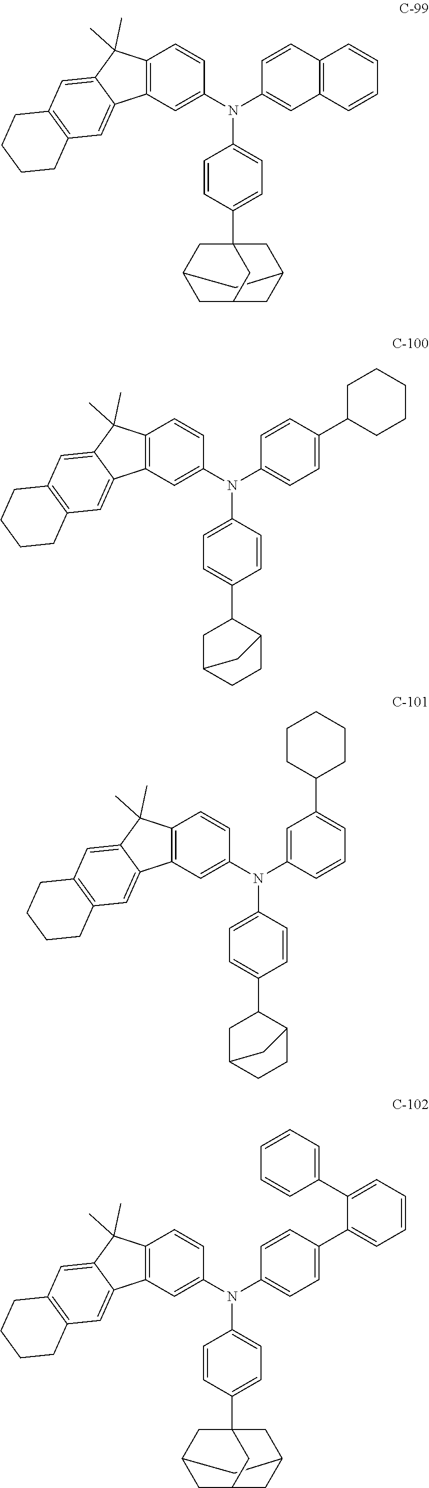 [SMILES: CC1(C)c2ccc(N(c3ccc(C45CC6CC(CC(C6)C4)C5)cc3)c3ccc4ccccc4c3)cc2-c2cc3c(cc21)CCCC3 ; CC1(C)c2ccc(N(c3ccc(C4CCCCC4)cc3)c3ccc(C4CC5CCC4C5)cc3)cc2-c2cc3c(cc21)CCCC3 ; CC1(C)c2ccc(N(c3ccc(C4CC5CCC4C5)cc3)c3cccc(C4CCCCC4)c3)cc2-c2cc3c(cc21)CCCC3 ; CC1(C)c2ccc(N(c3ccc(-c4ccccc4-c4ccccc4)cc3)c3ccc(C45CC6CC(CC(C6)C4)C5)cc3)cc2-c2cc3c(cc21)CCCC3]

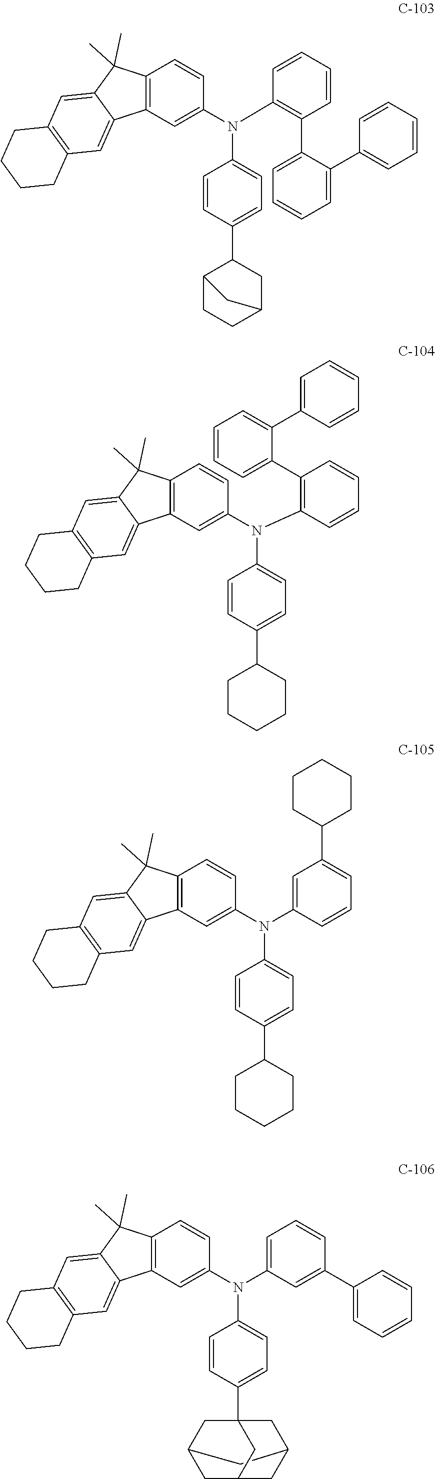 [SMILES: CC1(C)c2ccc(N(c3ccc(C4CC5CCC4C5)cc3)c3ccccc3-c3ccccc3-c3ccccc3)cc2-c2cc3c(cc21)CCCC3 ; CC1(C)c2ccc(N(c3ccc(C4CCCCC4)cc3)c3ccccc3-c3ccccc3-c3ccccc3)cc2-c2cc3c(cc21)CCCC3 ; CC1(C)c2ccc(N(c3ccc(C4CCCCC4)cc3)c3cccc(C4CCCCC4)c3)cc2-c2cc3c(cc21)CCCC3 ; CC1(C)c2ccc(N(c3ccc(C45CC6CC(CC(C6)C4)C5)cc3)c3cccc(-c4ccccc4)c3)cc2-c2cc3c(cc21)CCCC3]

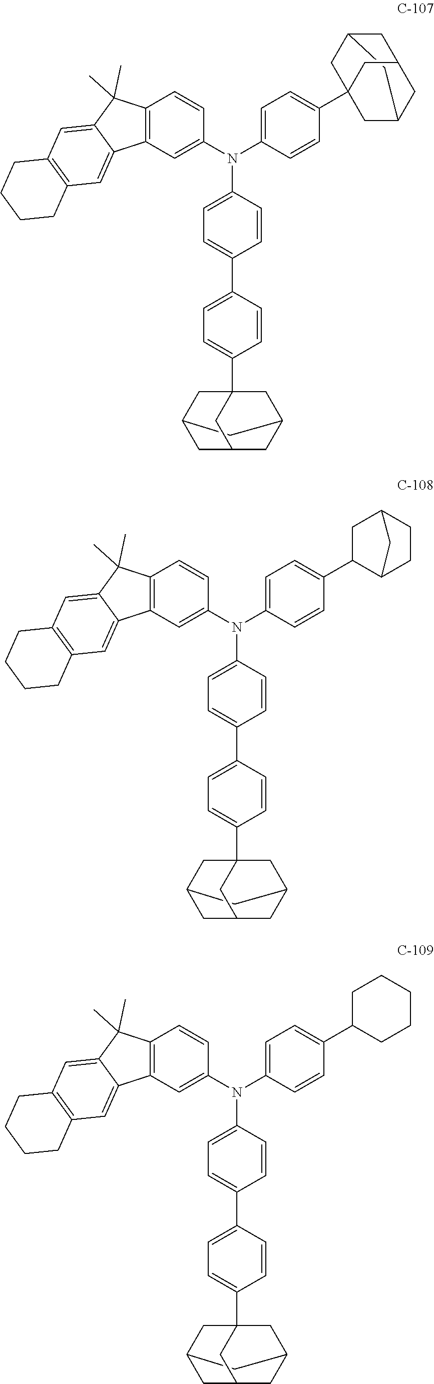 [SMILES: CC1(C)c2ccc(N(c3ccc(-c4ccc(C56CC7CC(CC(C7)C5)C6)cc4)cc3)c3ccc(C45CC6CC(CC(C6)C4)C5)cc3)cc2-c2cc3c(cc21)CCCC3 ; CC1(C)c2ccc(N(c3ccc(-c4ccc(C56CC7CC(CC(C7)C5)C6)cc4)cc3)c3ccc(C4CC5CCC4C5)cc3)cc2-c2cc3c(cc21)CCCC3 ; CC1(C)c2ccc(N(c3ccc(-c4ccc(C56CC7CC(CC(C7)C5)C6)cc4)cc3)c3ccc(C4CCCCC4)cc3)cc2-c2cc3c(cc21)CCCC3]

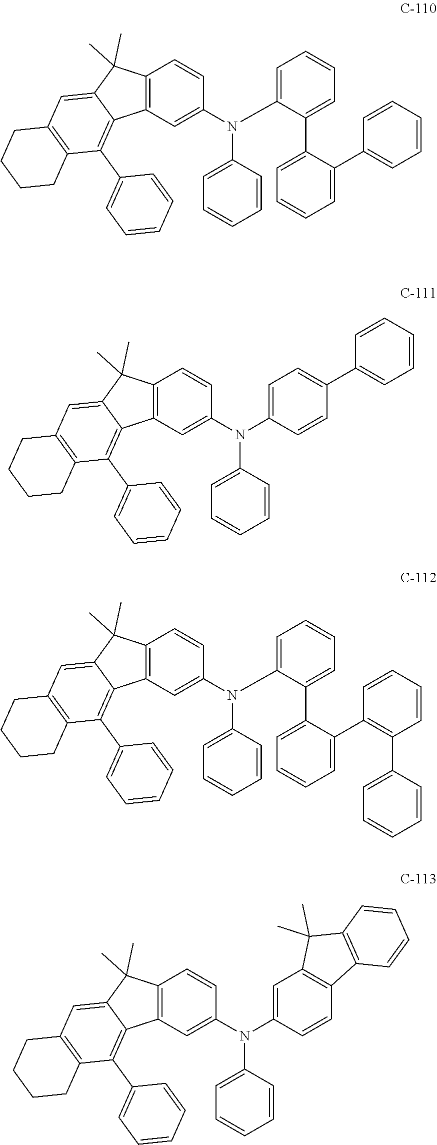 [SMILES: CC1(C)c2ccc(N(c3ccccc3)c3ccccc3-c3ccccc3-c3ccccc3)cc2-c2c1cc1c(c2-c2ccccc2)CCCC1 ; CC1(C)c2ccc(N(c3ccccc3)c3ccc(-c4ccccc4)cc3)cc2-c2c1cc1c(c2-c2ccccc2)CCCC1 ; CC1(C)c2ccc(N(c3ccccc3)c3ccccc3-c3ccccc3-c3ccccc3-c3ccccc3)cc2-c2c1cc1c(c2-c2ccccc2)CCCC1 ; CC1(C)c2ccccc2-c2ccc(N(c3ccccc3)c3ccc4c(c3)-c3c(cc5c(c3-c3ccccc3)CCCC5)C4(C)C)cc21]

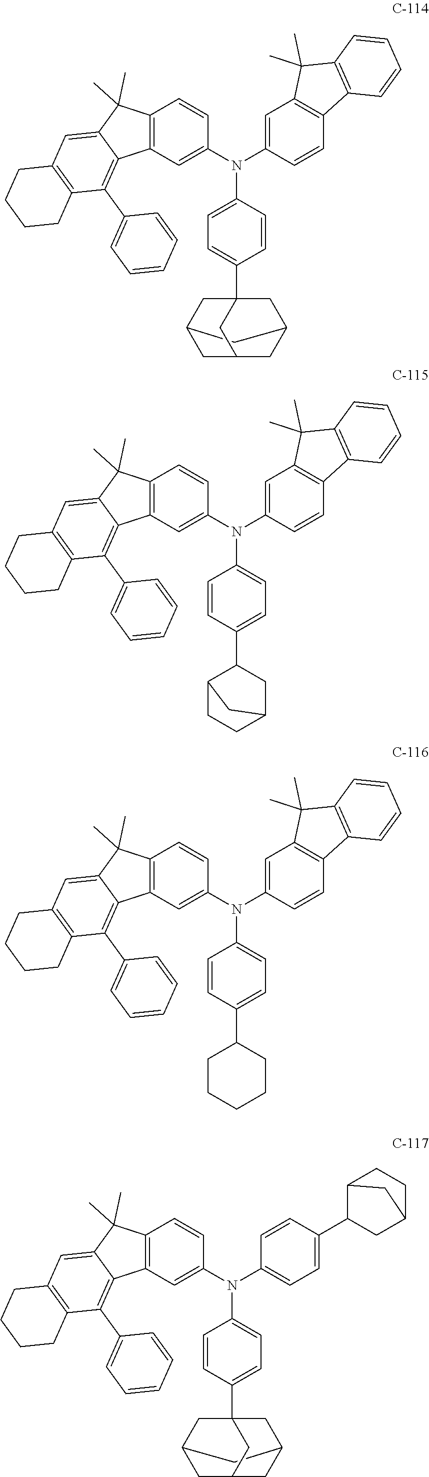 [SMILES: CC1(C)c2ccccc2-c2ccc(N(c3ccc(C45CC6CC(CC(C6)C4)C5)cc3)c3ccc4c(c3)-c3c(cc5c(c3-c3ccccc3)CCCC5)C4(C)C)cc21 ; CC1(C)c2ccccc2-c2ccc(N(c3ccc(C4CC5CCC4C5)cc3)c3ccc4c(c3)-c3c(cc5c(c3-c3ccccc3)CCCC5)C4(C)C)cc21 ; CC1(C)c2ccccc2-c2ccc(N(c3ccc(C4CCCCC4)cc3)c3ccc4c(c3)-c3c(cc5c(c3-c3ccccc3)CCCC5)C4(C)C)cc21 ; CC1(C)c2ccc(N(c3ccc(C4CC5CCC4C5)cc3)c3ccc(C45CC6CC(CC(C6)C4)C5)cc3)cc2-c2c1cc1c(c2-c2ccccc2)CCCC1]

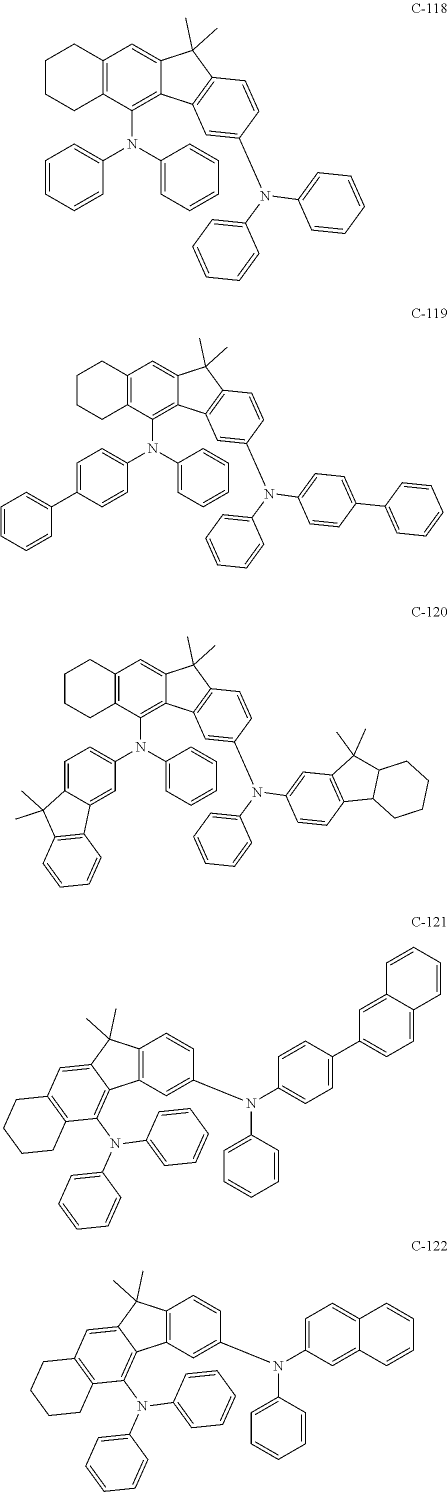 [SMILES: CC1(C)c2ccc(N(c3ccccc3)c3ccccc3)cc2-c2c1cc1c(c2N(c2ccccc2)c2ccccc2)CCCC1 ; CC1(C)c2ccc(N(c3ccccc3)c3ccc(-c4ccccc4)cc3)cc2-c2c1cc1c(c2N(c2ccccc2)c2ccc(-c3ccccc3)cc2)CCCC1 ; CC1(C)c2ccccc2-c2cc(N(c3ccccc3)c3c4c(cc5c3-c3cc(N(c6ccccc6)c6ccc7c(c6)C(C)(C)C6CCCCC76)ccc3C5(C)C)CCCC4)ccc21 ; CC1(C)c2ccc(N(c3ccccc3)c3ccc(-c4ccc5ccccc5c4)cc3)cc2-c2c1cc1c(c2N(c2ccccc2)c2ccccc2)CCCC1 ; CC1(C)c2ccc(N(c3ccccc3)c3ccc4ccccc4c3)cc2-c2c1cc1c(c2N(c2ccccc2)c2ccccc2)CCCC1]

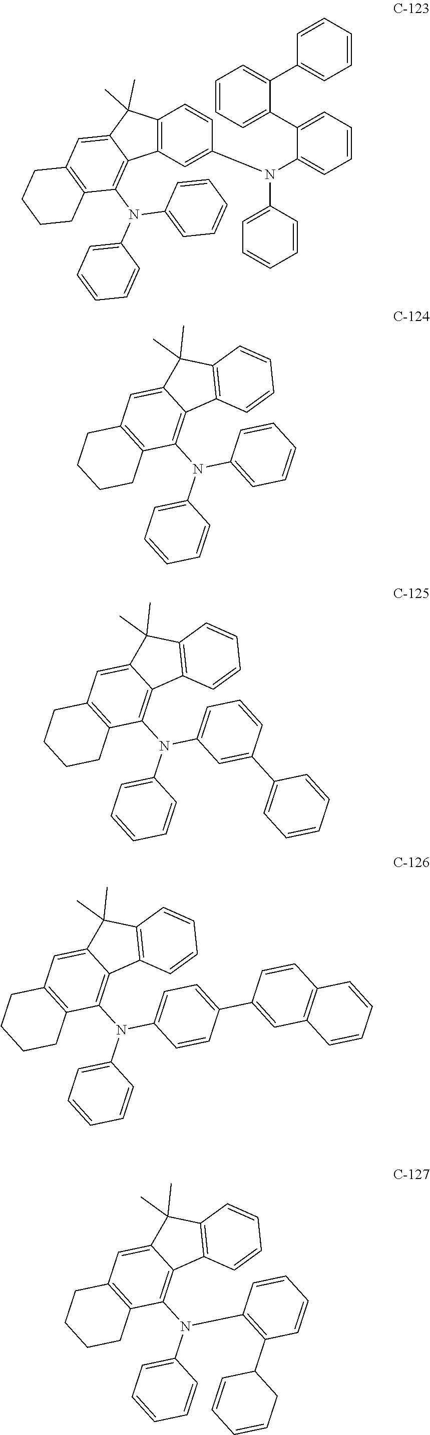

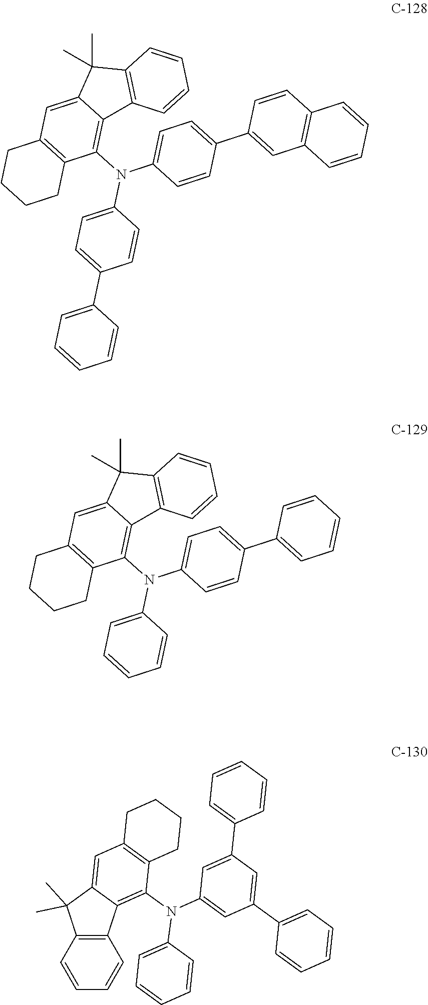 [SMILES: CC1(C)c2ccccc2-c2c1cc1c(c2N(c2ccc(-c3ccccc3)cc2)c2ccc(-c3ccc4ccccc4c3)cc2)CCCC1 ; CC1(C)c2ccccc2-c2c1cc1c(c2N(c2ccccc2)c2ccc(-c3ccccc3)cc2)CCCC1 ; CC1(C)c2ccccc2-c2c1cc1c(c2N(c2ccccc2)c2cc(-c3ccccc3)cc(-c3ccccc3)c2)CCCC1]

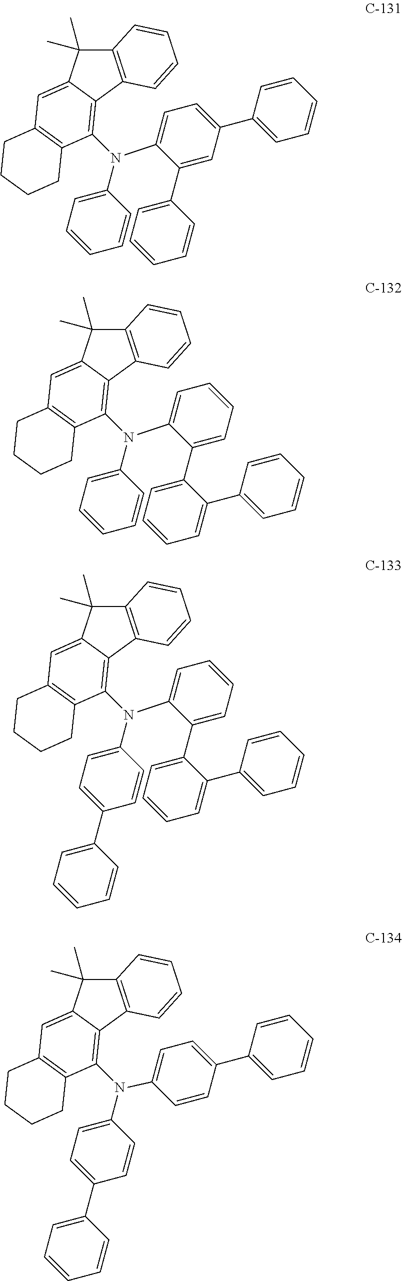

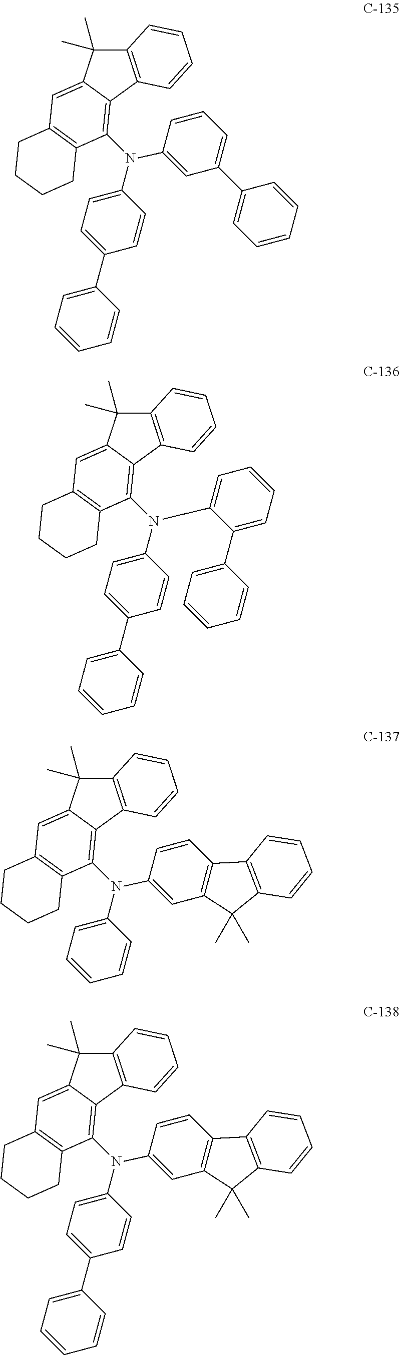

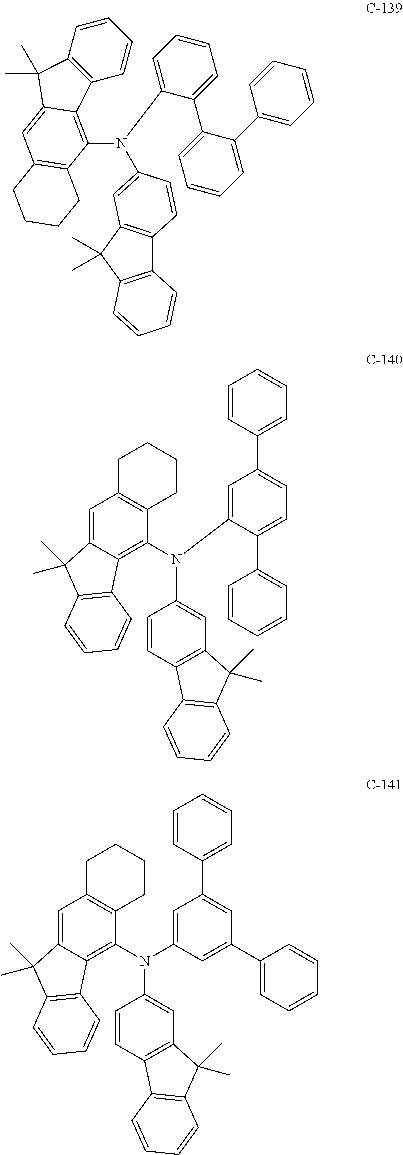

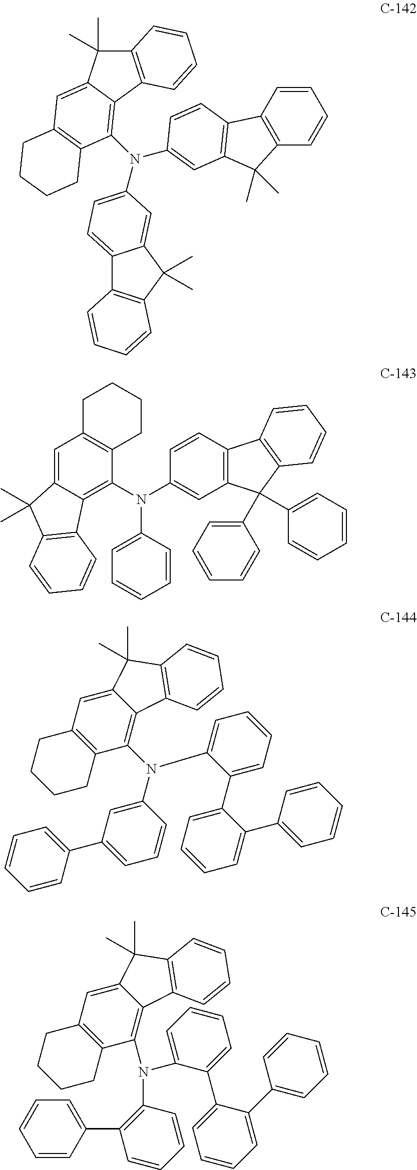

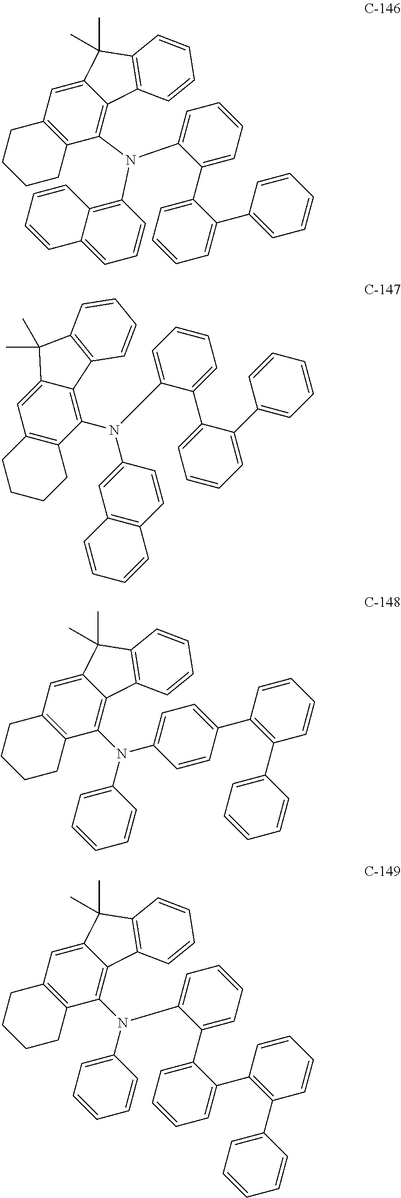

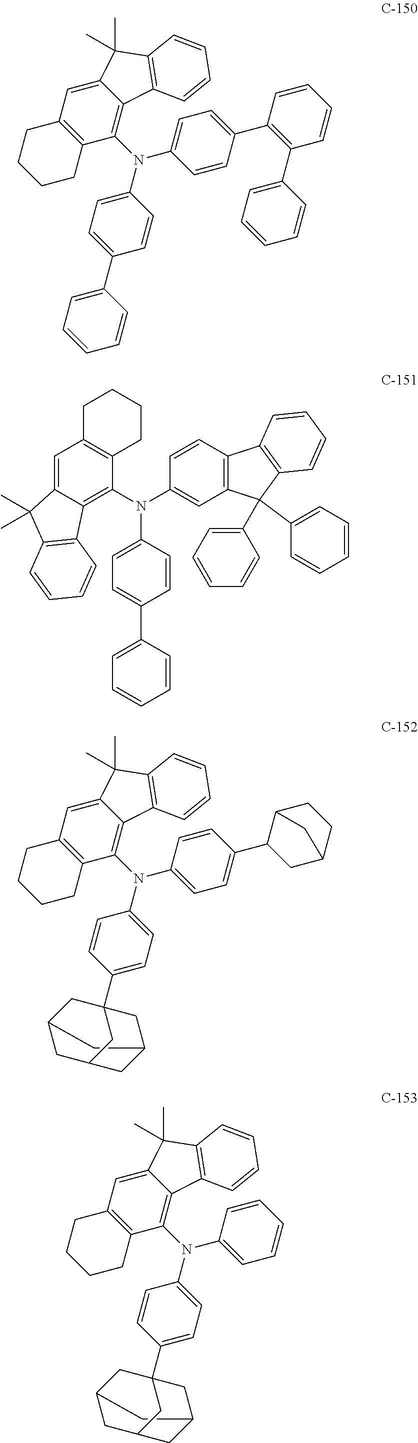

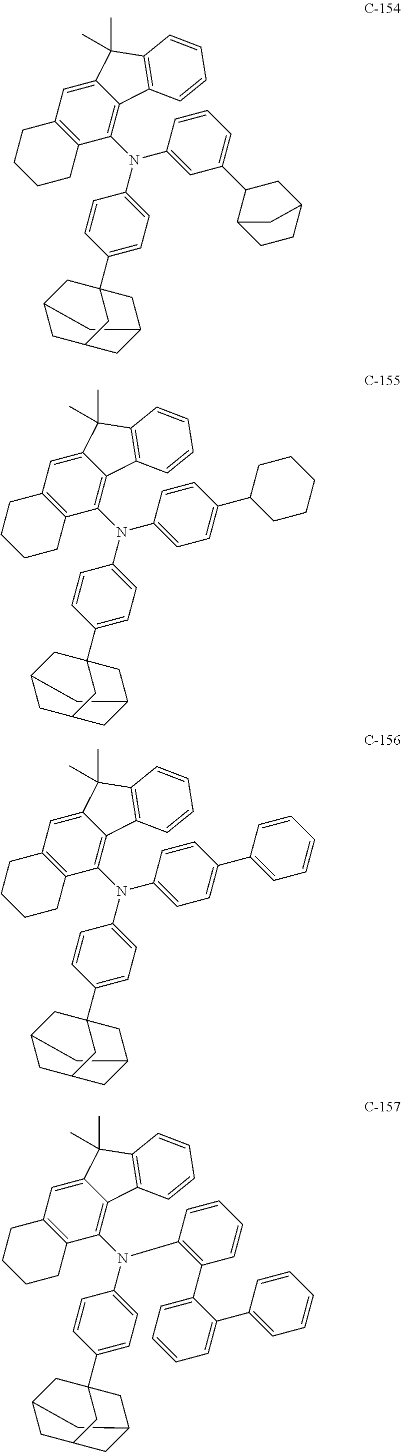

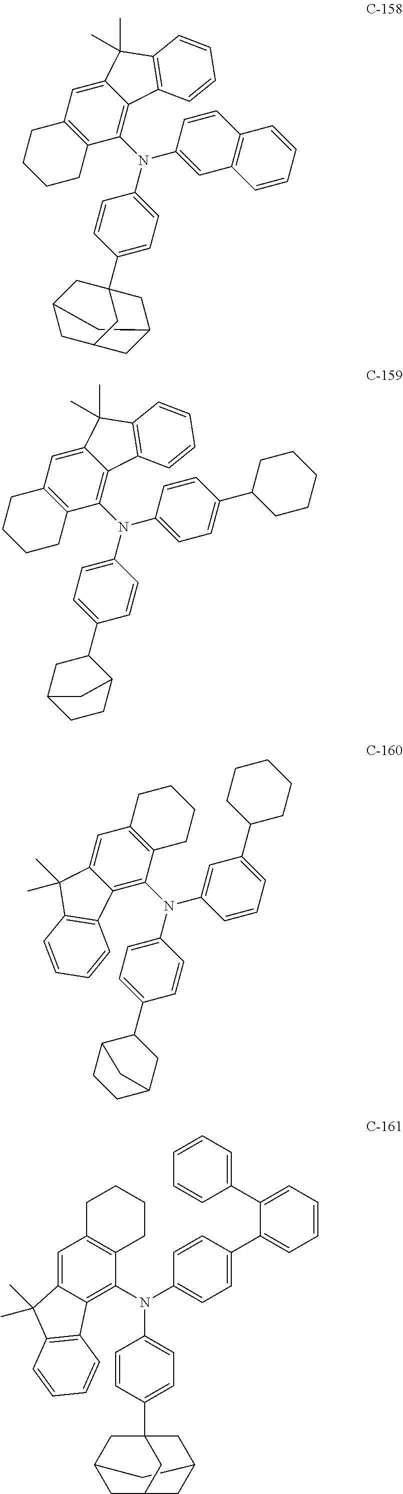

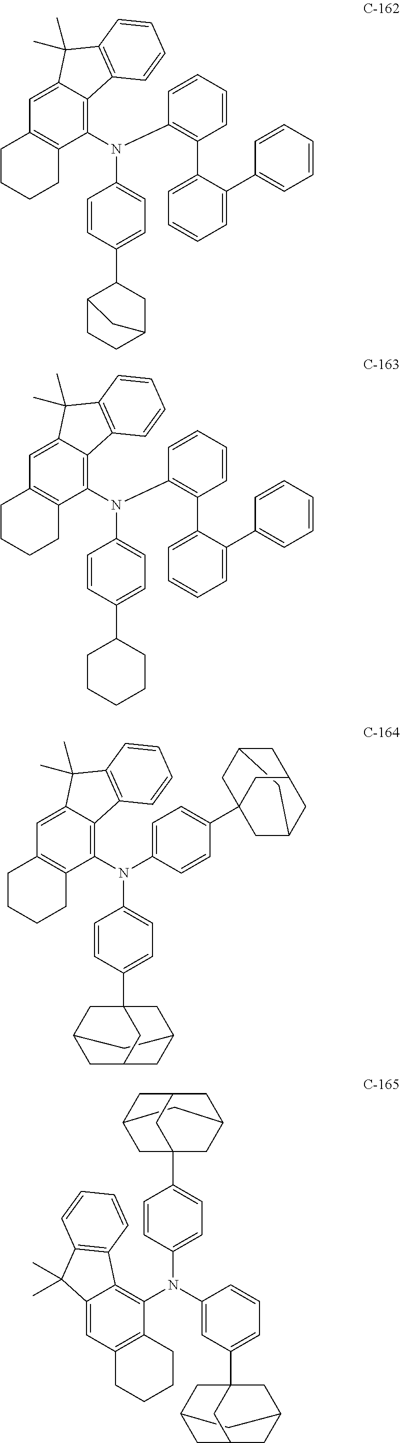

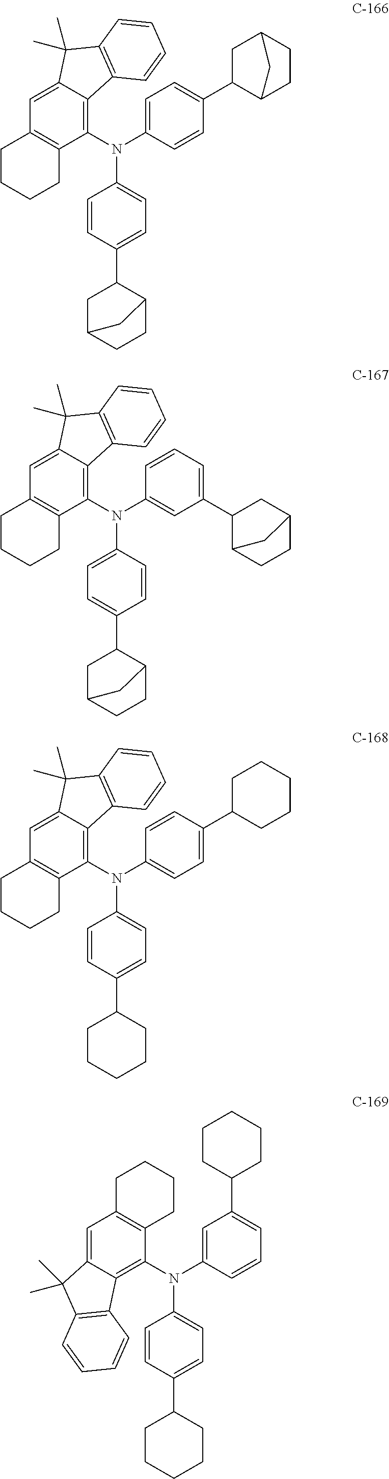

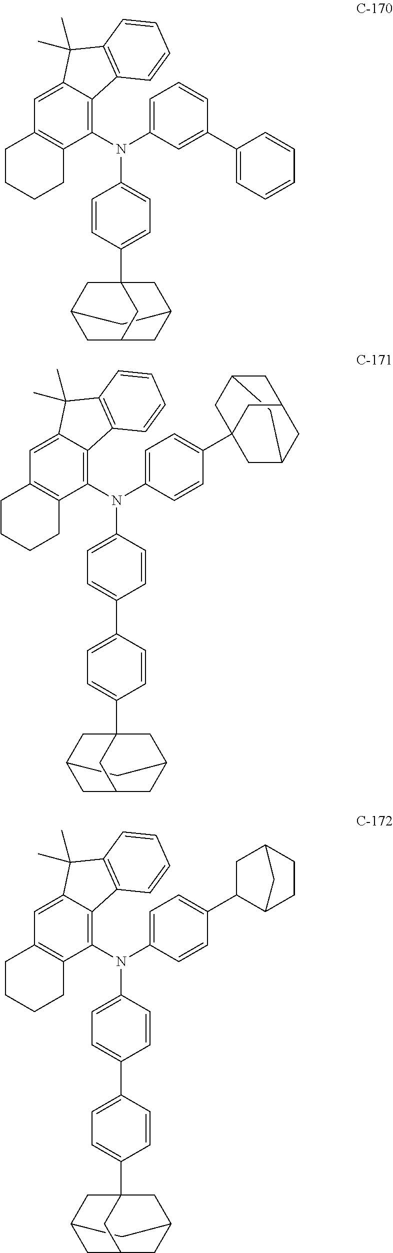

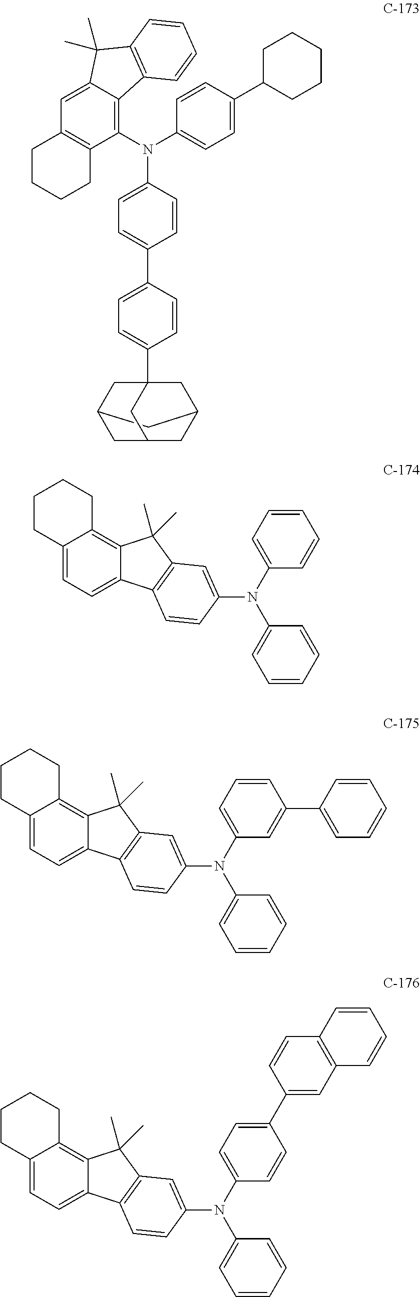 [SMILES: CC1(C)c2ccccc2-c2c1cc1c(c2N(c2ccc(-c3ccc(C45CC6CC(CC(C6)C4)C5)cc3)cc2)c2ccc(C3CCCCC3)cc2)CCCC1 ; CC1(C)c2cc(N(c3ccccc3)c3ccccc3)ccc2-c2ccc3c(c21)CCCC3 ; CC1(C)c2cc(N(c3ccccc3)c3cccc(-c4ccccc4)c3)ccc2-c2ccc3c(c21)CCCC3 ; CC1(C)c2cc(N(c3ccccc3)c3ccc(-c4ccc5ccccc5c4)cc3)ccc2-c2ccc3c(c21)CCCC3]

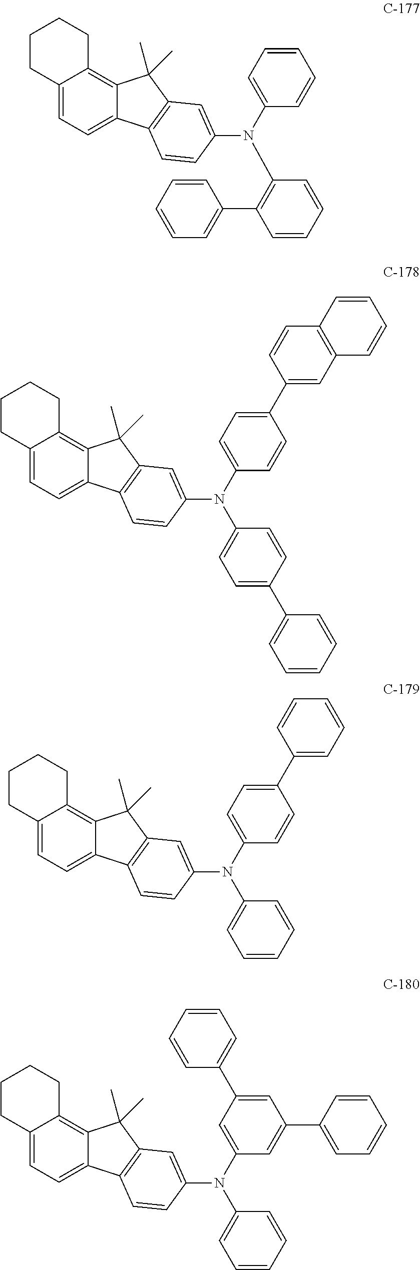

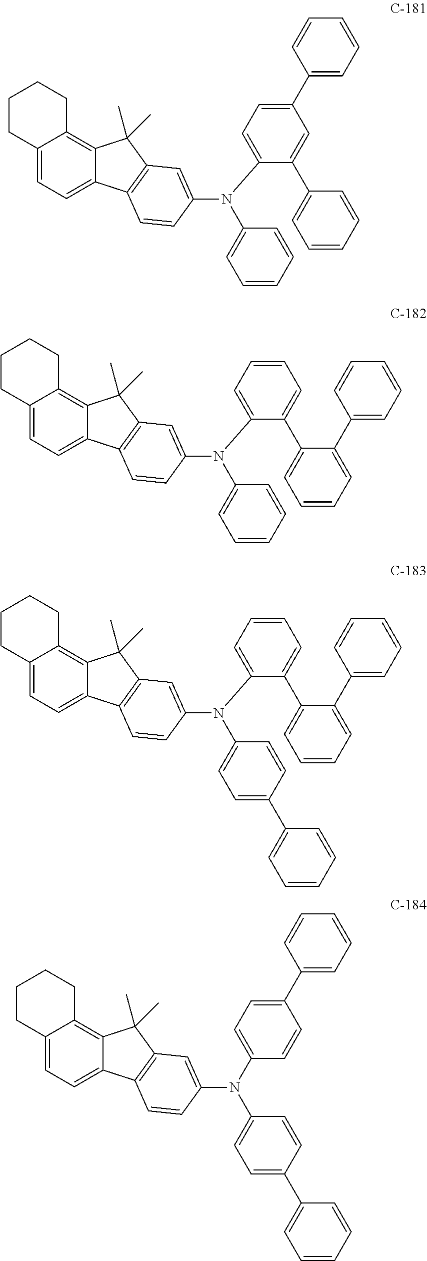

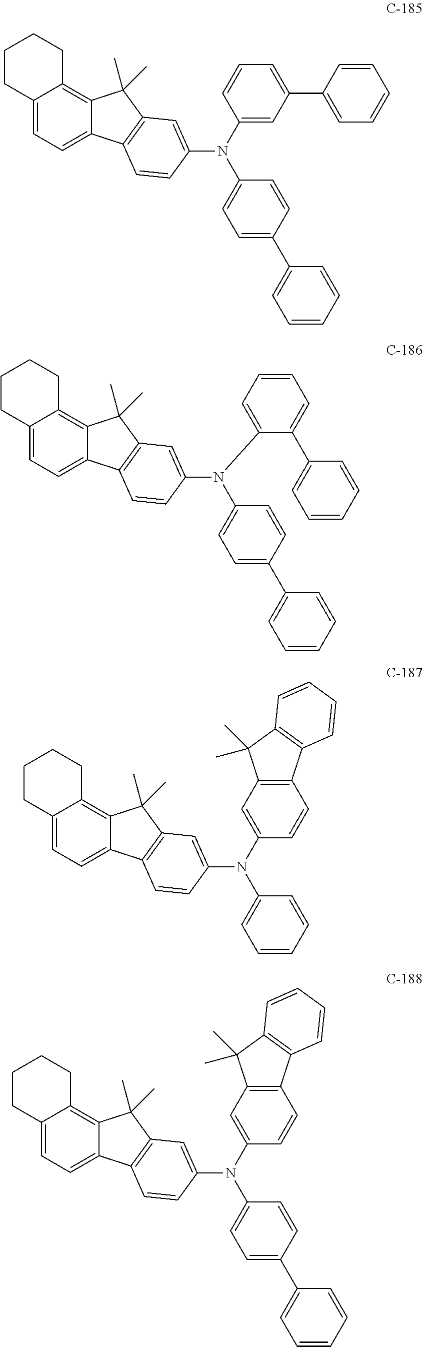

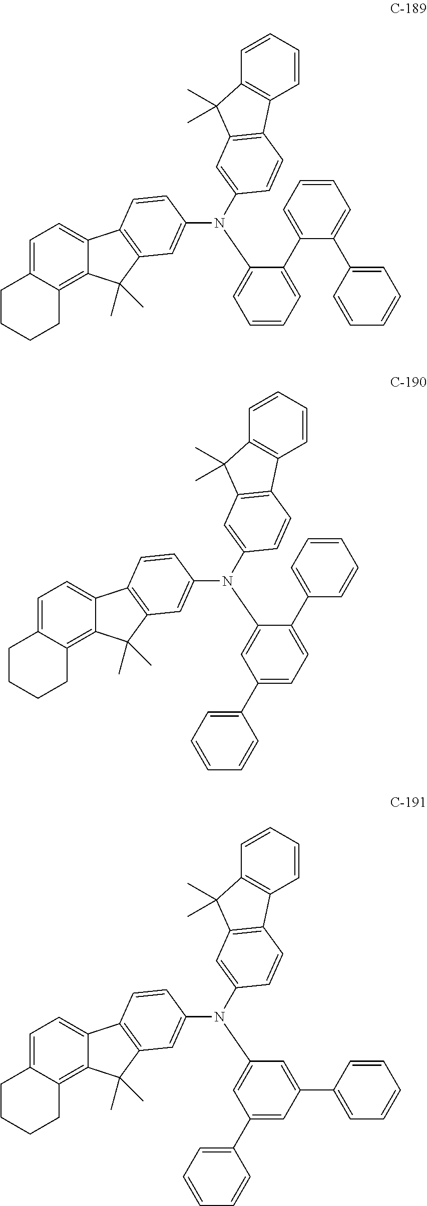 [SMILES: CC1(C)c2ccccc2-c2ccc(N(c3ccc4c(c3)C(C)(C)c3c-4ccc4c3CCCC4)c3ccccc3-c3ccccc3-c3ccccc3)cc21 ; CC1(C)c2ccccc2-c2ccc(N(c3ccc4c(c3)C(C)(C)c3c-4ccc4c3CCCC4)c3cc(-c4ccccc4)ccc3-c3ccccc3)cc21 ; CC1(C)c2ccccc2-c2ccc(N(c3cc(-c4ccccc4)cc(-c4ccccc4)c3)c3ccc4c(c3)C(C)(C)c3c-4ccc4c3CCCC4)cc21]

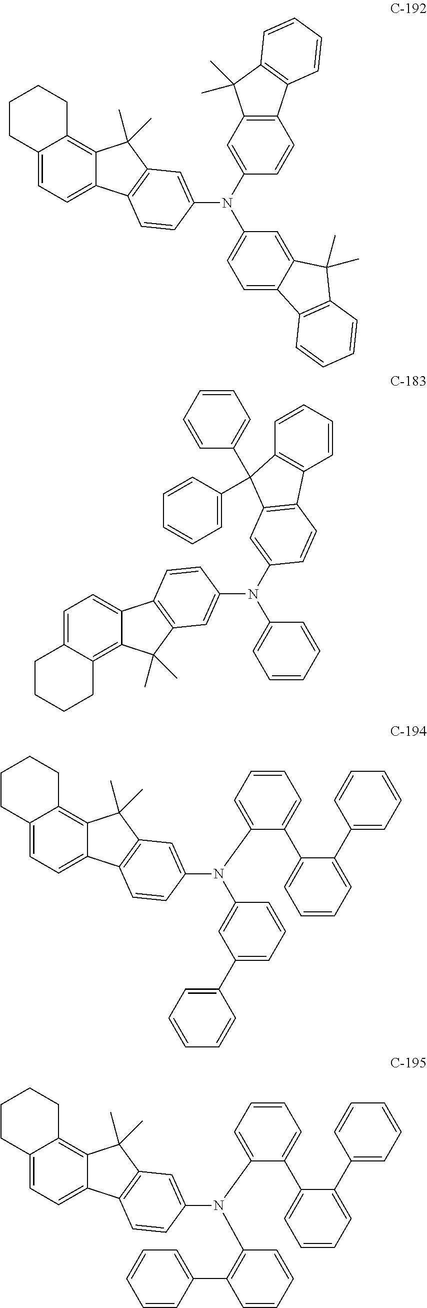

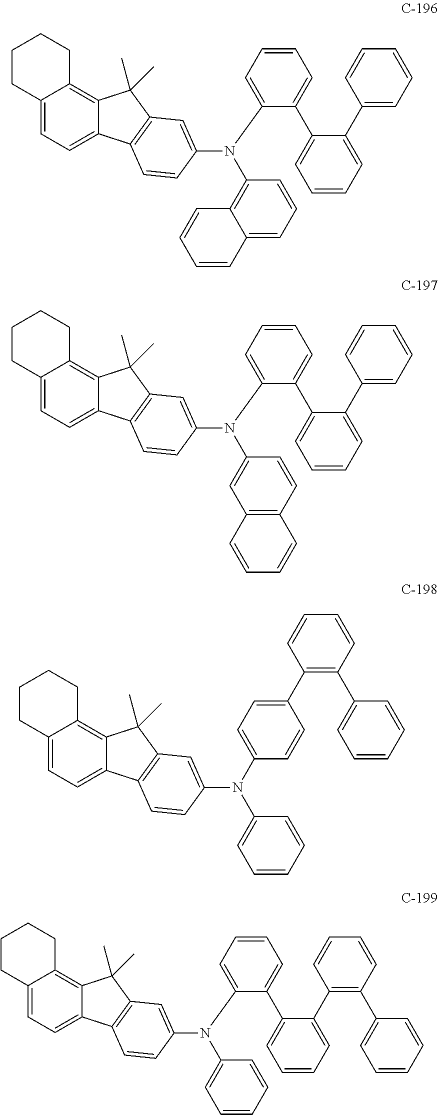 [SMILES: CC1(C)c2cc(N(c3ccccc3-c3ccccc3-c3ccccc3)c3cccc4ccccc34)ccc2-c2ccc3c(c21)CCCC3 ; CC1(C)c2cc(N(c3ccc4ccccc4c3)c3ccccc3-c3ccccc3-c3ccccc3)ccc2-c2ccc3c(c21)CCCC3 ; CC1(C)c2cc(N(c3ccccc3)c3ccc(-c4ccccc4-c4ccccc4)cc3)ccc2-c2ccc3c(c21)CCCC3 ; CC1(C)c2cc(N(c3ccccc3)c3ccccc3-c3ccccc3-c3ccccc3-c3ccccc3)ccc2-c2ccc3c(c21)CCCC3]

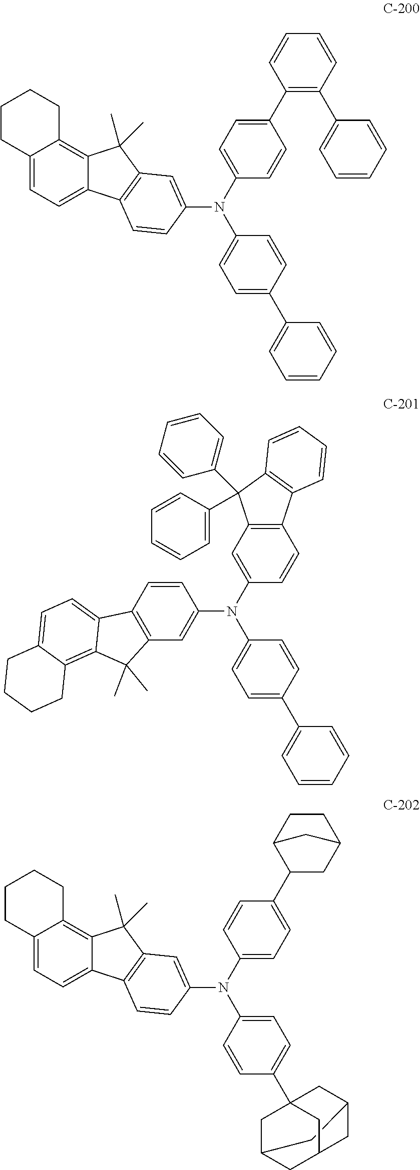

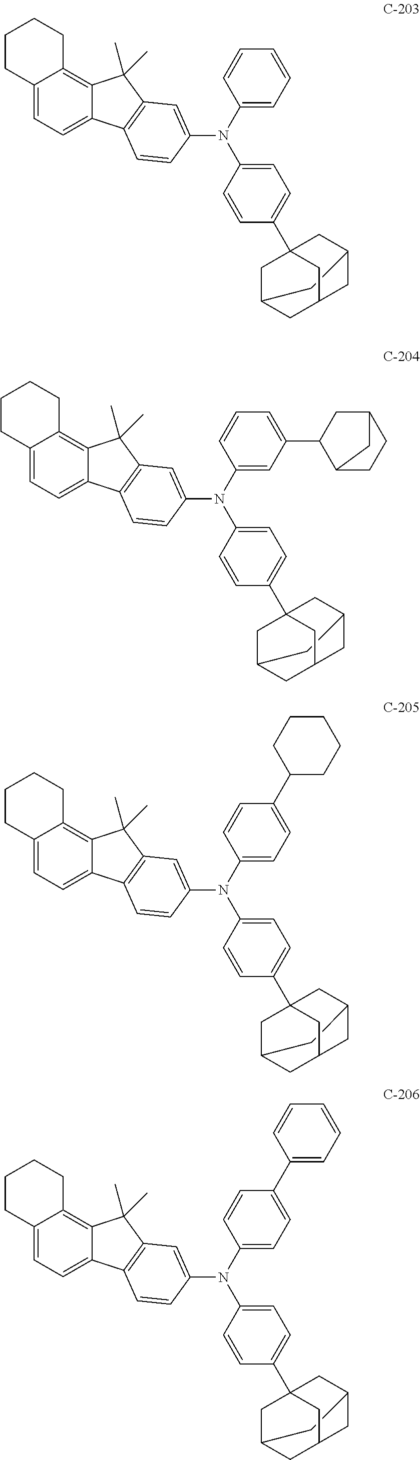

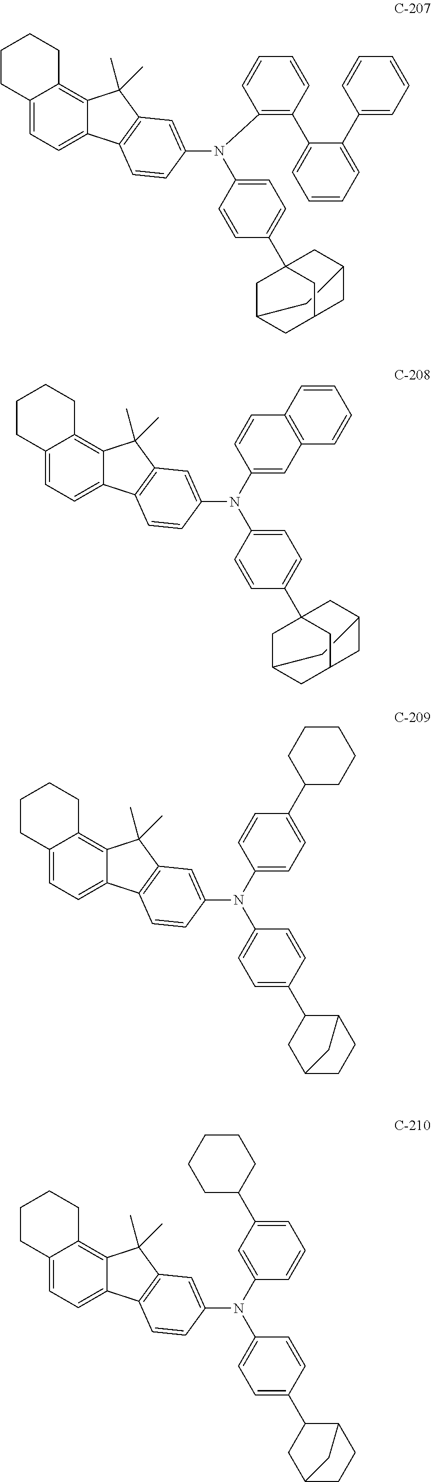

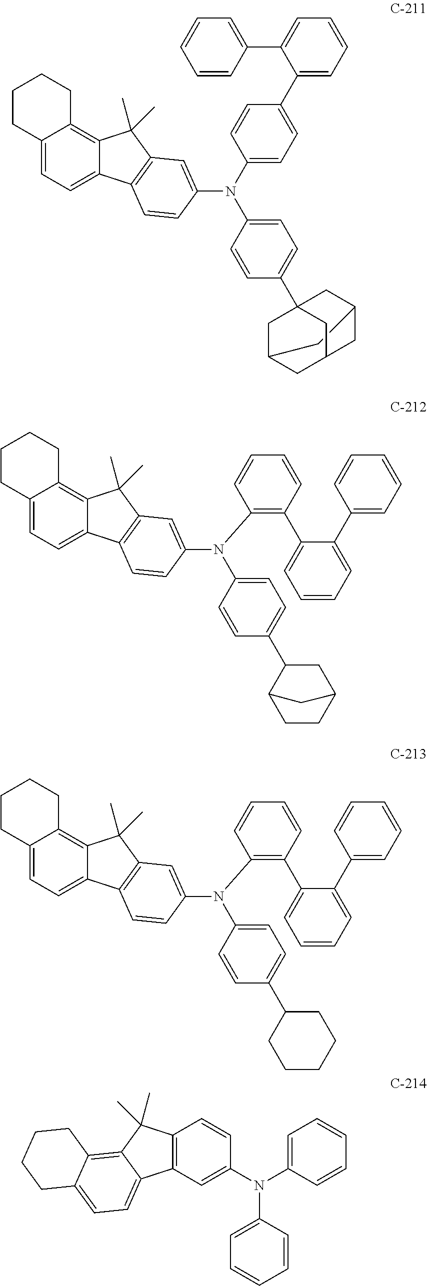 [SMILES: CC1(C)c2cc(N(c3ccc(-c4ccccc4-c4ccccc4)cc3)c3ccc(C45CC6CC(CC(C6)C4)C5)cc3)ccc2-c2ccc3c(c21)CCCC3 ; CC1(C)c2cc(N(c3ccc(C4CC5CCC4C5)cc3)c3ccccc3-c3ccccc3-c3ccccc3)ccc2-c2ccc3c(c21)CCCC3 ; CC1(C)c2cc(N(c3ccc(C4CCCCC4)cc3)c3ccccc3-c3ccccc3-c3ccccc3)ccc2-c2ccc3c(c21)CCCC3 ; CC1(C)c2ccc(N(c3ccccc3)c3ccccc3)cc2-c2ccc3c(c21)CCCC3]

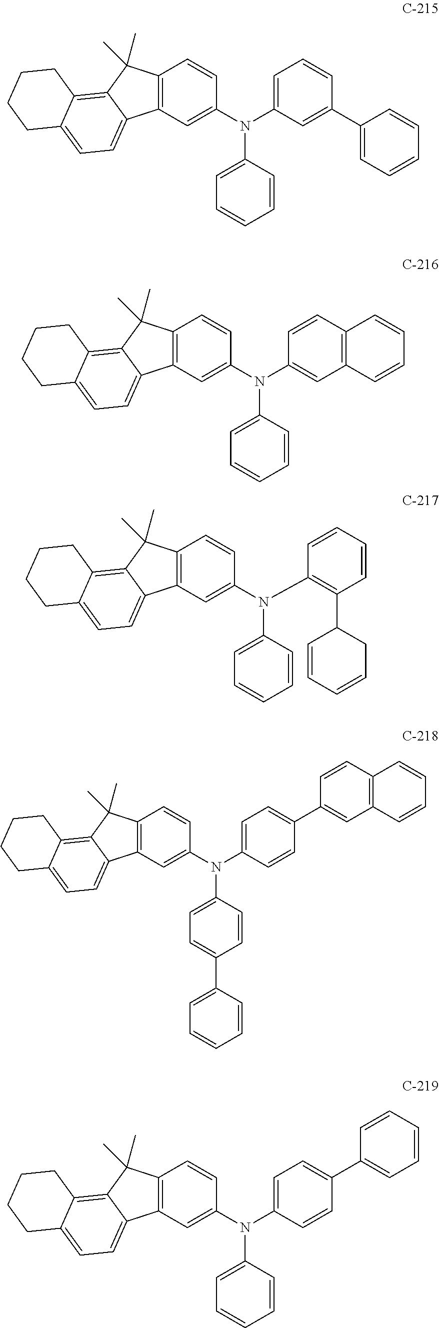 [SMILES: CC1(C)c2ccc(N(c3ccccc3)c3cccc(-c4ccccc4)c3)cc2-c2ccc3c(c21)CCCC3 ; CC1(C)c2ccc(N(c3ccccc3)c3ccc4ccccc4c3)cc2-c2ccc3c(c21)CCCC3 ; CC1(C)c2ccc(N(c3ccccc3)c3ccccc3C3C=CC=CC3)cc2-c2ccc3c(c21)CCCC3 ; CC1(C)c2ccc(N(c3ccc(-c4ccccc4)cc3)c3ccc(-c4ccc5ccccc5c4)cc3)cc2-c2ccc3c(c21)CCCC3 ; CC1(C)c2ccc(N(c3ccccc3)c3ccc(-c4ccccc4)cc3)cc2-c2ccc3c(c21)CCCC3]

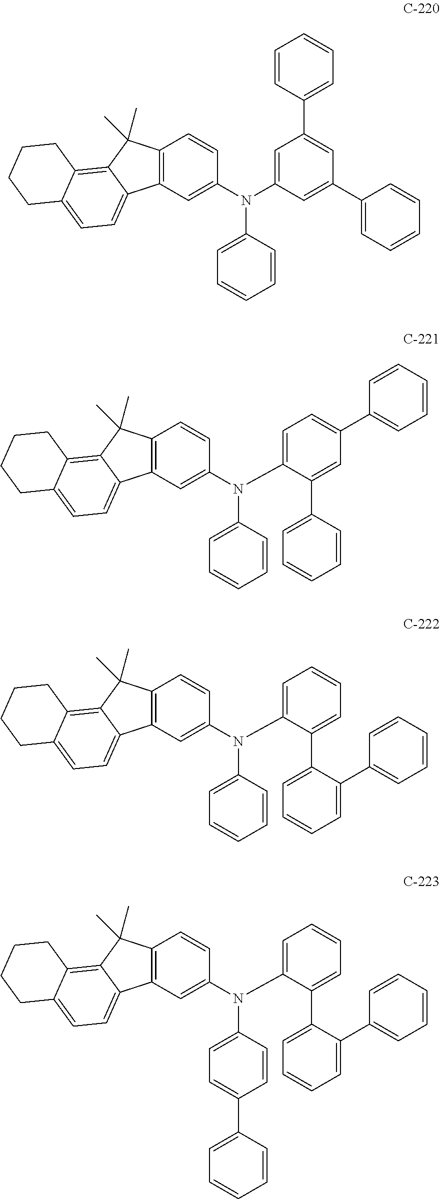 [SMILES: CC1(C)c2ccc(N(c3ccccc3)c3cc(-c4ccccc4)cc(-c4ccccc4)c3)cc2-c2ccc3c(c21)CCCC3 ; CC1(C)c2ccc(N(c3ccccc3)c3ccc(-c4ccccc4)cc3-c3ccccc3)cc2-c2ccc3c(c21)CCCC3 ; CC1(C)c2ccc(N(c3ccccc3)c3ccccc3-c3ccccc3-c3ccccc3)cc2-c2ccc3c(c21)CCCC3 ; CC1(C)c2ccc(N(c3ccc(-c4ccccc4)cc3)c3ccccc3-c3ccccc3-c3ccccc3)cc2-c2ccc3c(c21)CCCC3]

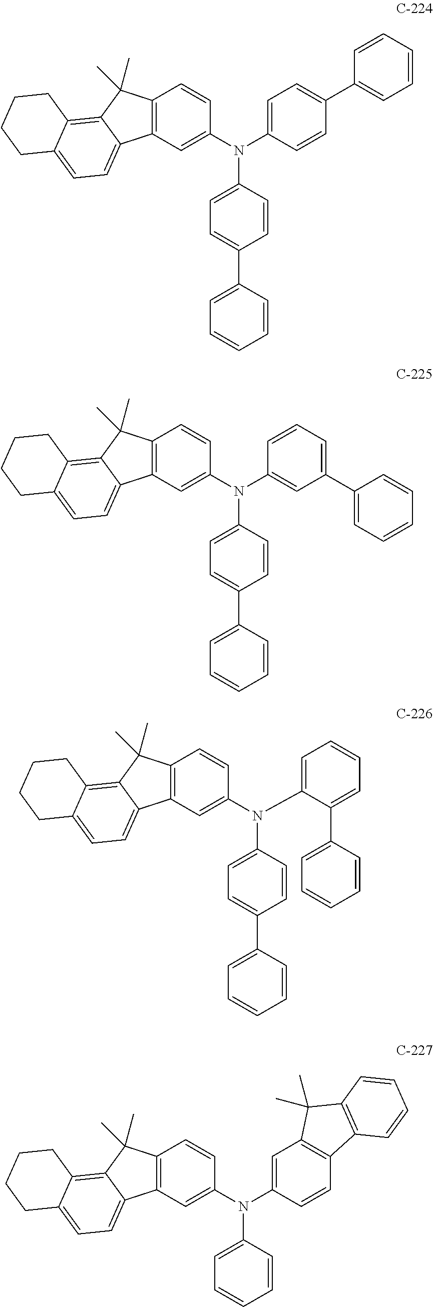 [SMILES: CC1(C)c2ccc(N(c3ccc(-c4ccccc4)cc3)c3ccc(-c4ccccc4)cc3)cc2-c2ccc3c(c21)CCCC3 ; CC1(C)c2ccc(N(c3ccc(-c4ccccc4)cc3)c3cccc(-c4ccccc4)c3)cc2-c2ccc3c(c21)CCCC3 ; CC1(C)c2ccc(N(c3ccc(-c4ccccc4)cc3)c3ccccc3-c3ccccc3)cc2-c2ccc3c(c21)CCCC3 ; CC1(C)c2ccccc2-c2ccc(N(c3ccccc3)c3ccc4c(c3)-c3ccc5c(c3C4(C)C)CCCC5)cc21]

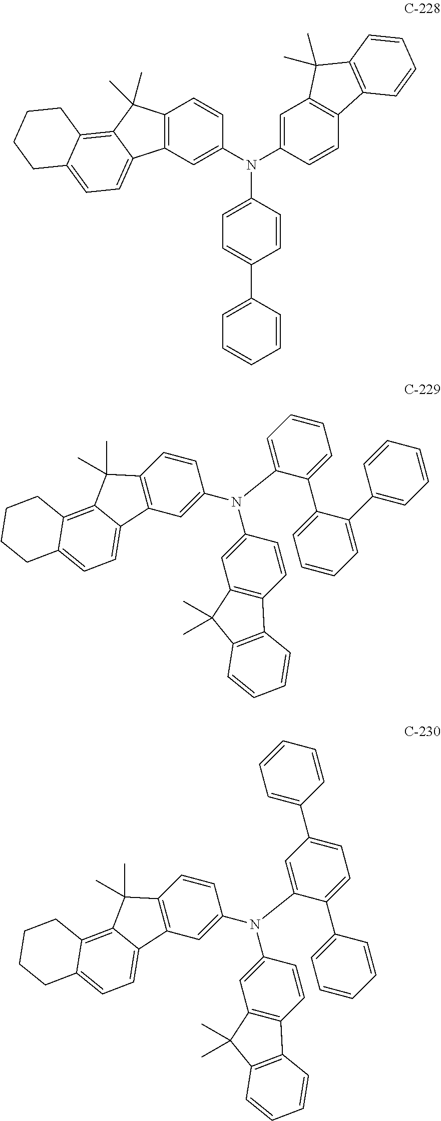 [SMILES: CC1(C)c2ccccc2-c2ccc(N(c3ccc(-c4ccccc4)cc3)c3ccc4c(c3)-c3ccc5c(c3C4(C)C)CCCC5)cc21 ; CC1(C)c2ccccc2-c2ccc(N(c3ccc4c(c3)-c3ccc5c(c3C4(C)C)CCCC5)c3ccccc3-c3ccccc3-c3ccccc3)cc21 ; CC1(C)c2ccccc2-c2ccc(N(c3ccc4c(c3)-c3ccc5c(c3C4(C)C)CCCC5)c3cc(-c4ccccc4)ccc3-c3ccccc3)cc21]

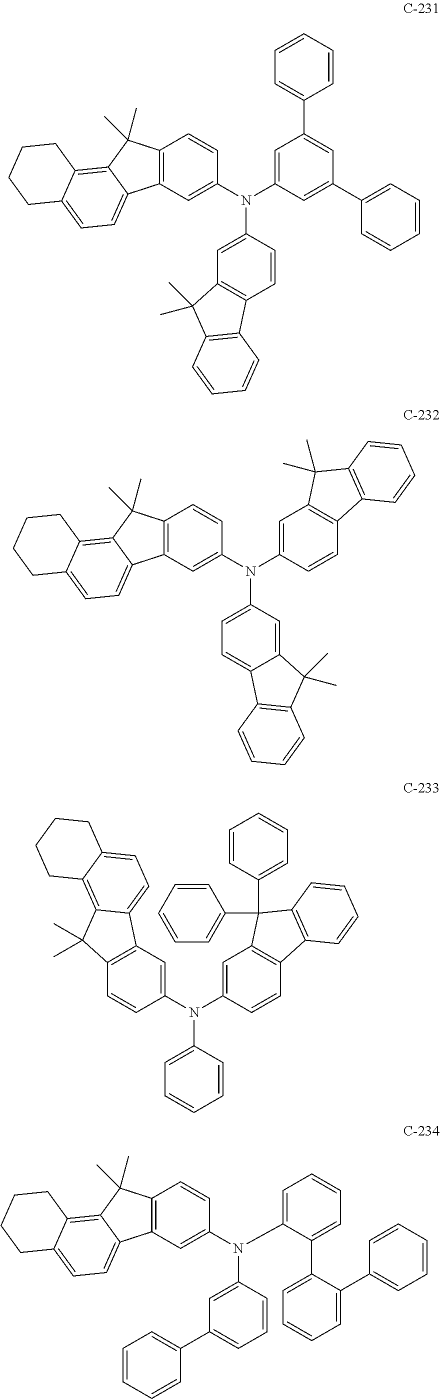 [SMILES: CC1(C)c2ccccc2-c2ccc(N(c3cc(-c4ccccc4)cc(-c4ccccc4)c3)c3ccc4c(c3)-c3ccc5c(c3C4(C)C)CCCC5)cc21 ; CC1(C)c2ccccc2-c2ccc(N(c3ccc4c(c3)-c3ccc5c(c3C4(C)C)CCCC5)c3ccc4c(c3)C(C)(C)c3ccccc3-4)cc21 ; CC1(C)c2ccc(N(c3ccccc3)c3ccc4c(c3)C(c3ccccc3)(c3ccccc3)c3ccccc3-4)cc2-c2ccc3c(c21)CCCC3 ; CC1(C)c2ccc(N(c3cccc(-c4ccccc4)c3)c3ccccc3-c3ccccc3-c3ccccc3)cc2-c2ccc3c(c21)CCCC3]

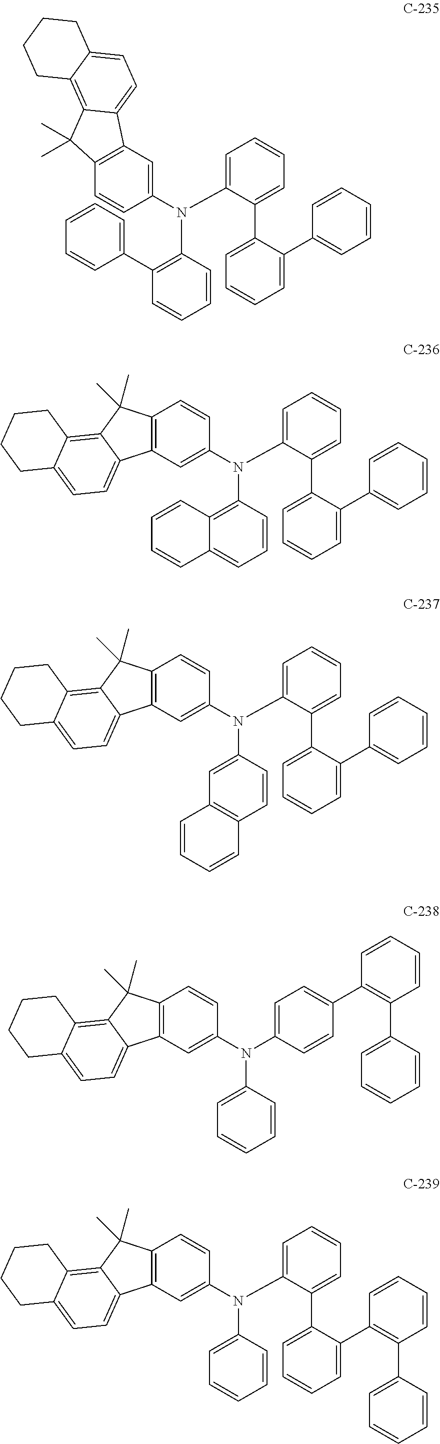 [SMILES: CC1(C)c2ccc(N(c3ccccc3-c3ccccc3)c3ccccc3-c3ccccc3-c3ccccc3)cc2-c2ccc3c(c21)CCCC3 ; CC1(C)c2ccc(N(c3ccccc3-c3ccccc3-c3ccccc3)c3cccc4ccccc34)cc2-c2ccc3c(c21)CCCC3 ; CC1(C)c2ccc(N(c3ccc4ccccc4c3)c3ccccc3-c3ccccc3-c3ccccc3)cc2-c2ccc3c(c21)CCCC3 ; CC1(C)c2ccc(N(c3ccccc3)c3ccc(-c4ccccc4-c4ccccc4)cc3)cc2-c2ccc3c(c21)CCCC3 ; CC1(C)c2ccc(N(c3ccccc3)c3ccccc3-c3ccccc3-c3ccccc3-c3ccccc3)cc2-c2ccc3c(c21)CCCC3]

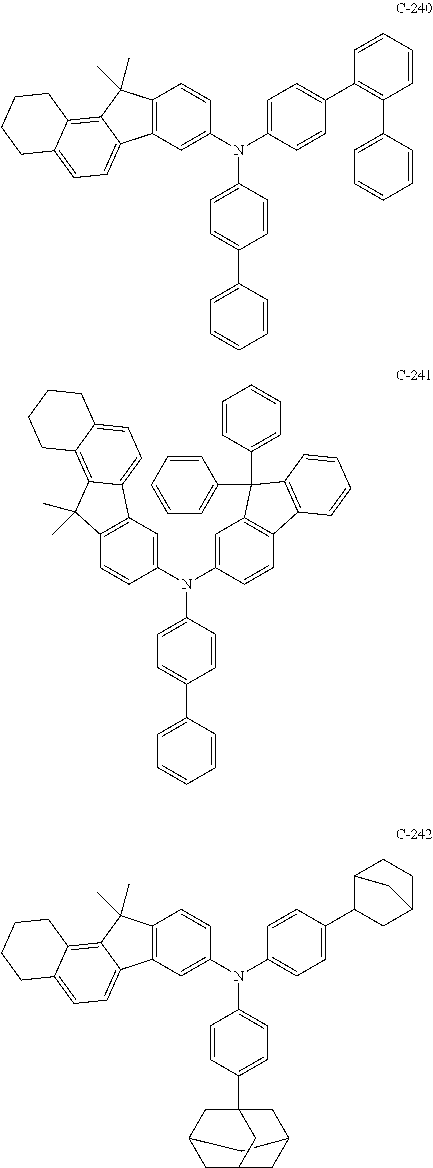

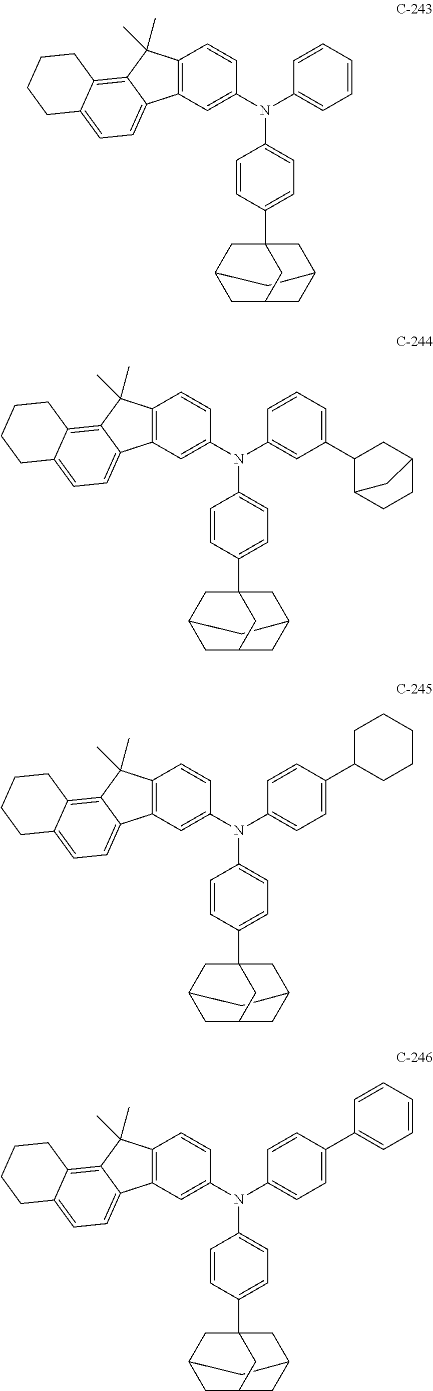 [SMILES: CC1(C)c2ccc(N(c3ccccc3)c3ccc(C45CC6CC(CC(C6)C4)C5)cc3)cc2-c2ccc3c(c21)CCCC3 ; CC1(C)c2ccc(N(c3ccc(C45CC6CC(CC(C6)C4)C5)cc3)c3cccc(C4CC5CCC4C5)c3)cc2-c2ccc3c(c21)CCCC3 ; CC1(C)c2ccc(N(c3ccc(C4CCCCC4)cc3)c3ccc(C45CC6CC(CC(C6)C4)C5)cc3)cc2-c2ccc3c(c21)CCCC3 ; CC1(C)c2ccc(N(c3ccc(-c4ccccc4)cc3)c3ccc(C45CC6CC(CC(C6)C4)C5)cc3)cc2-c2ccc3c(c21)CCCC3]

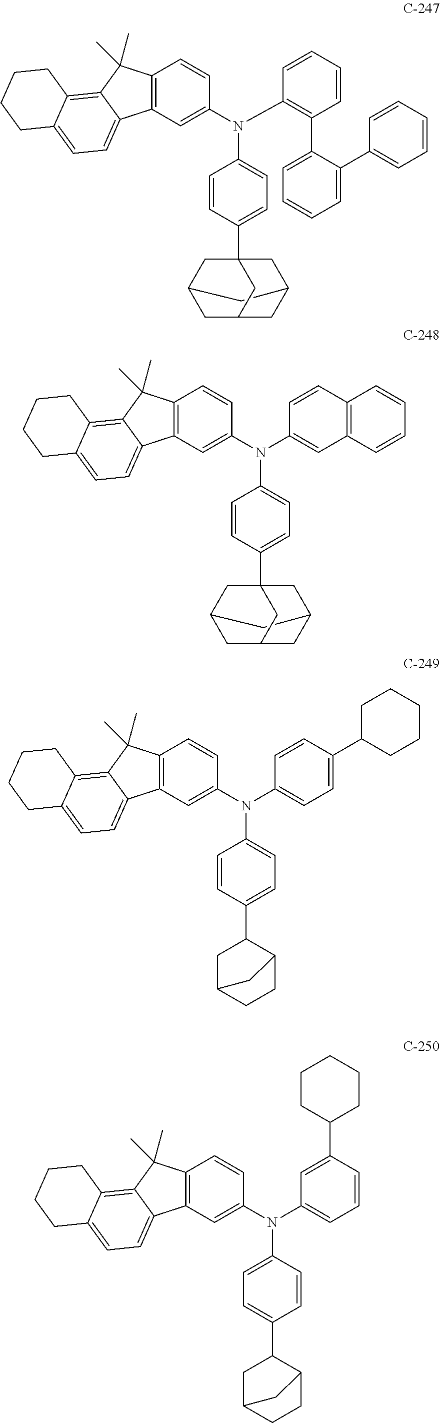 [SMILES: CC1(C)c2ccc(N(c3ccc(C45CC6CC(CC(C6)C4)C5)cc3)c3ccccc3-c3ccccc3-c3ccccc3)cc2-c2ccc3c(c21)CCCC3 ; CC1(C)c2ccc(N(c3ccc(C45CC6CC(CC(C6)C4)C5)cc3)c3ccc4ccccc4c3)cc2-c2ccc3c(c21)CCCC3 ; CC1(C)c2ccc(N(c3ccc(C4CCCCC4)cc3)c3ccc(C4CC5CCC4C5)cc3)cc2-c2ccc3c(c21)CCCC3 ; CC1(C)c2ccc(N(c3ccc(C4CC5CCC4C5)cc3)c3cccc(C4CCCCC4)c3)cc2-c2ccc3c(c21)CCCC3]

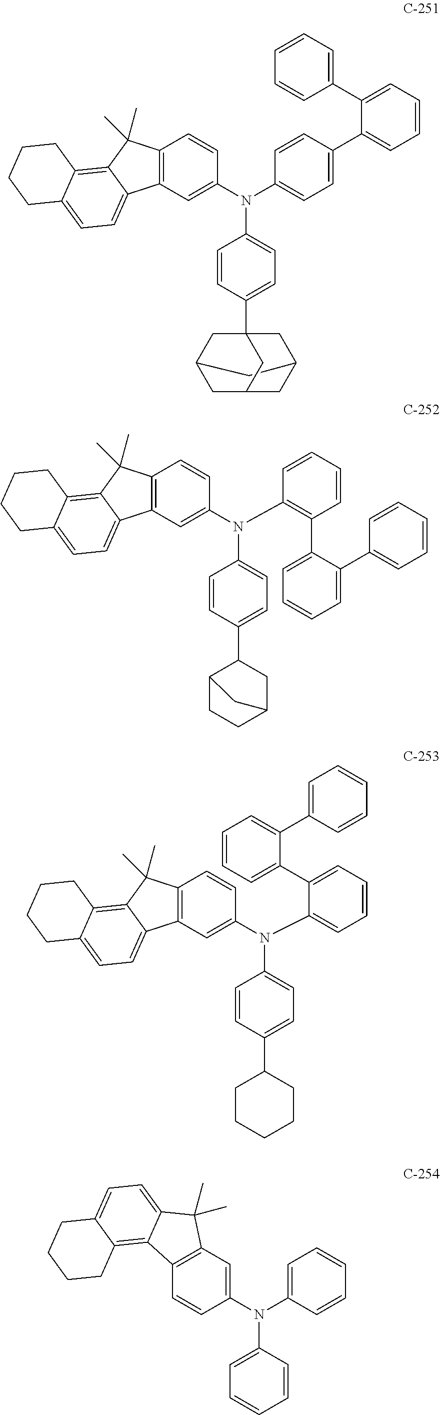 [SMILES: CC1(C)c2ccc(N(c3ccc(-c4ccccc4-c4ccccc4)cc3)c3ccc(C45CC6CC(CC(C6)C4)C5)cc3)cc2-c2ccc3c(c21)CCCC3 ; CC1(C)c2ccc(N(c3ccc(C4CC5CCC4C5)cc3)c3ccccc3-c3ccccc3-c3ccccc3)cc2-c2ccc3c(c21)CCCC3 ; CC1(C)c2ccc(N(c3ccc(C4CCCCC4)cc3)c3ccccc3-c3ccccc3-c3ccccc3)cc2-c2ccc3c(c21)CCCC3 ; CC1(C)c2cc(N(c3ccccc3)c3ccccc3)ccc2-c2c1ccc1c2CCCC1]

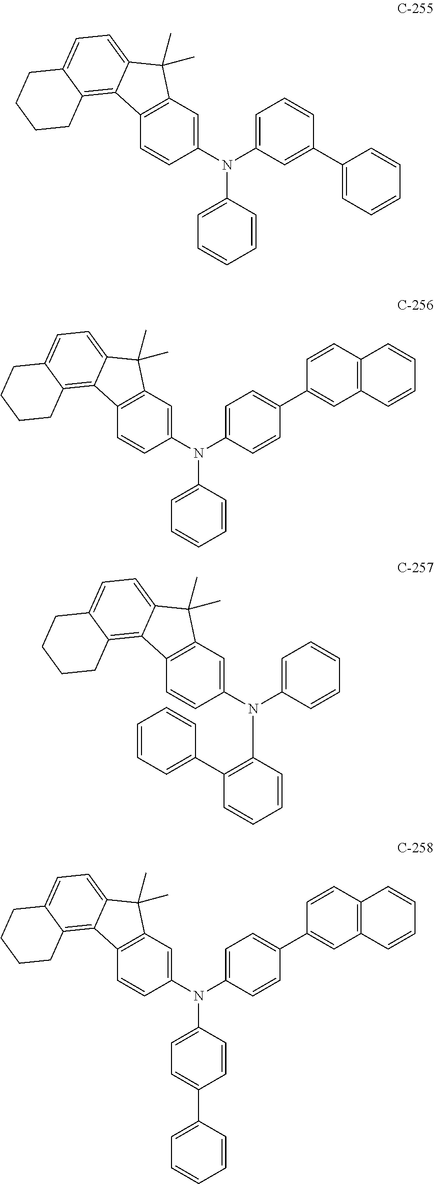

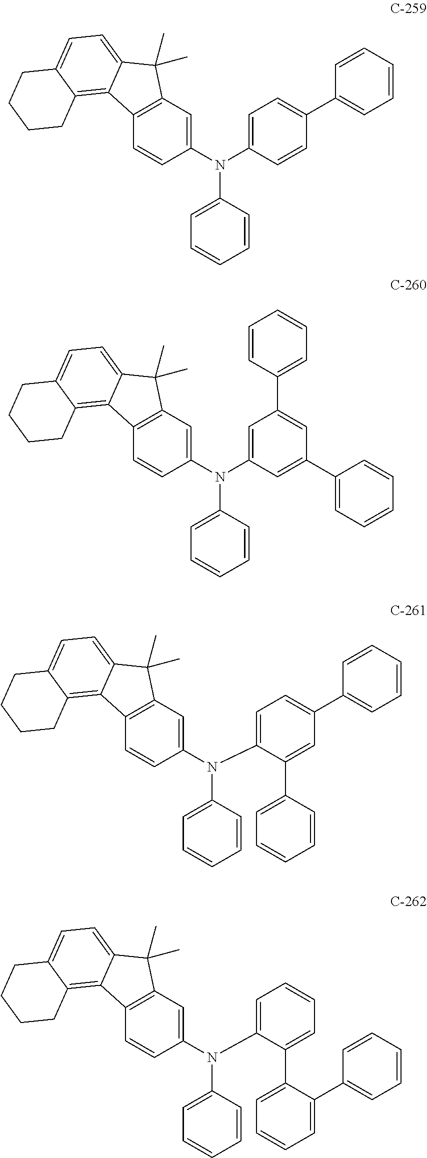 [SMILES: CC1(C)c2cc(N(c3ccccc3)c3ccc(-c4ccccc4)cc3)ccc2-c2c1ccc1c2CCCC1 ; CC1(C)c2cc(N(c3ccccc3)c3cc(-c4ccccc4)cc(-c4ccccc4)c3)ccc2-c2c1ccc1c2CCCC1 ; CC1(C)c2cc(N(c3ccccc3)c3ccc(-c4ccccc4)cc3-c3ccccc3)ccc2-c2c1ccc1c2CCCC1 ; CC1(C)c2cc(N(c3ccccc3)c3ccccc3-c3ccccc3-c3ccccc3)ccc2-c2c1ccc1c2CCCC1]

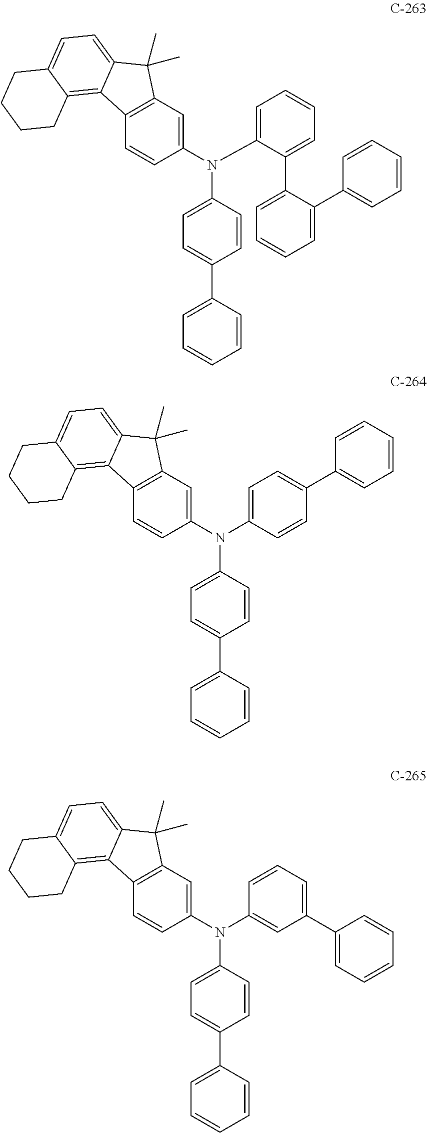 [SMILES: CC1(C)c2cc(N(c3ccc(-c4ccccc4)cc3)c3ccccc3-c3ccccc3-c3ccccc3)ccc2-c2c1ccc1c2CCCC1 ; CC1(C)c2cc(N(c3ccc(-c4ccccc4)cc3)c3ccc(-c4ccccc4)cc3)ccc2-c2c1ccc1c2CCCC1 ; CC1(C)c2cc(N(c3ccc(-c4ccccc4)cc3)c3cccc(-c4ccccc4)c3)ccc2-c2c1ccc1c2CCCC1]

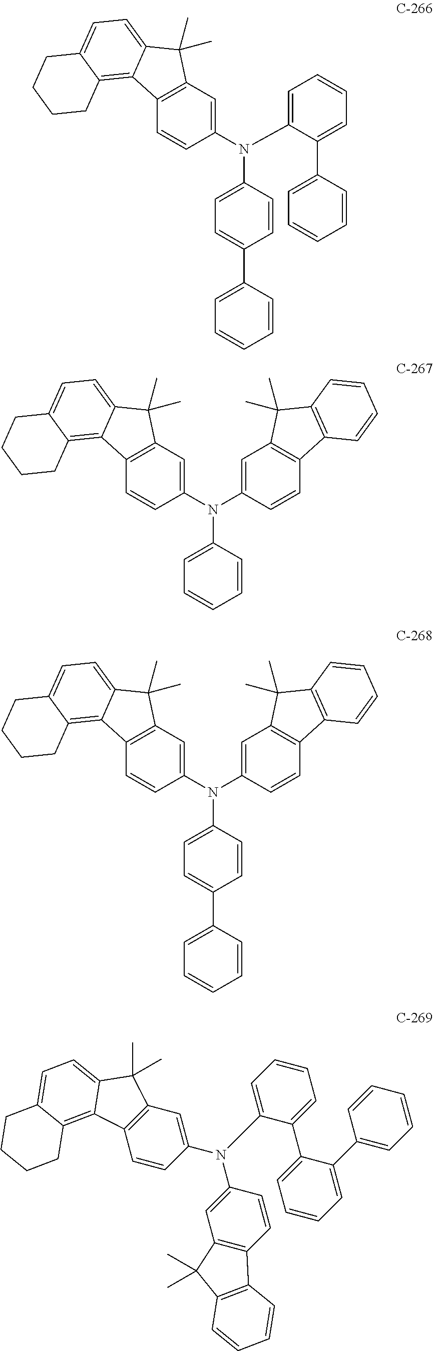

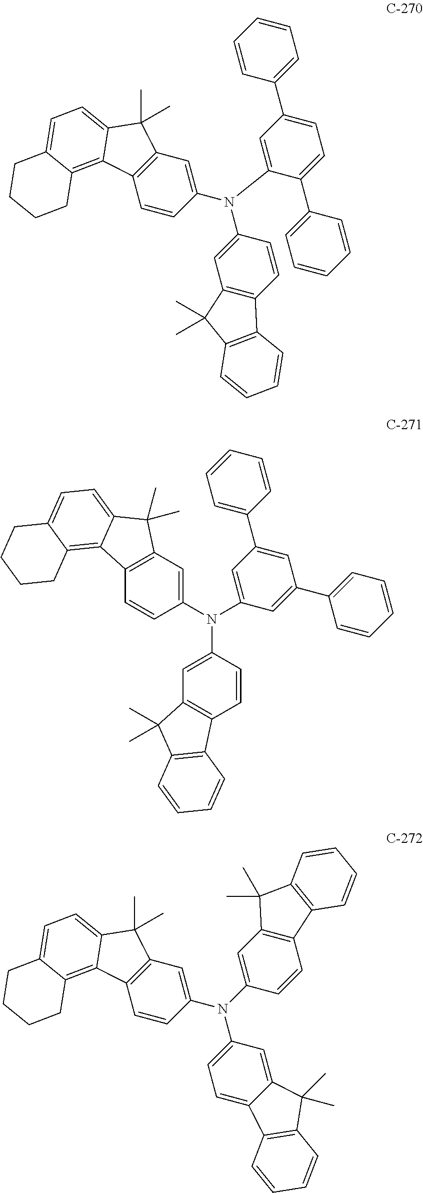 [SMILES: CC1(C)c2ccccc2-c2ccc(N(c3ccc4c(c3)C(C)(C)c3ccc5c(c3-4)CCCC5)c3cc(-c4ccccc4)ccc3-c3ccccc3)cc21 ; CC1(C)c2ccccc2-c2ccc(N(c3cc(-c4ccccc4)cc(-c4ccccc4)c3)c3ccc4c(c3)C(C)(C)c3ccc5c(c3-4)CCCC5)cc21 ; CC1(C)c2ccccc2-c2ccc(N(c3ccc4c(c3)C(C)(C)c3ccccc3-4)c3ccc4c(c3)C(C)(C)c3ccc5c(c3-4)CCCC5)cc21]

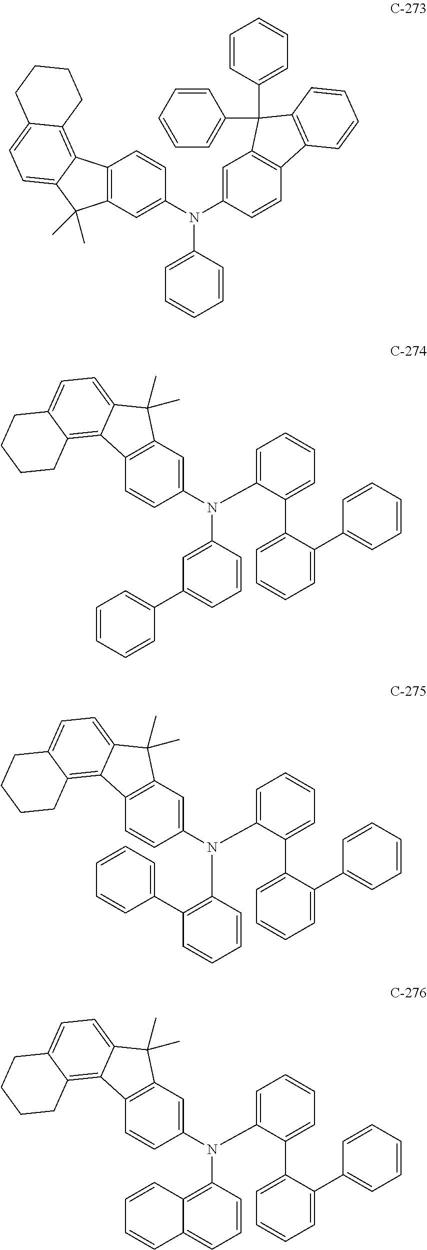 [SMILES: CC1(C)c2cc(N(c3ccccc3)c3ccc4c(c3)C(c3ccccc3)(c3ccccc3)c3ccccc3-4)ccc2-c2c1ccc1c2CCCC1 ; CC1(C)c2cc(N(c3cccc(-c4ccccc4)c3)c3ccccc3-c3ccccc3-c3ccccc3)ccc2-c2c1ccc1c2CCCC1 ; CC1(C)c2cc(N(c3ccccc3-c3ccccc3)c3ccccc3-c3ccccc3-c3ccccc3)ccc2-c2c1ccc1c2CCCC1 ; CC1(C)c2cc(N(c3ccccc3-c3ccccc3-c3ccccc3)c3cccc4ccccc34)ccc2-c2c1ccc1c2CCCC1]

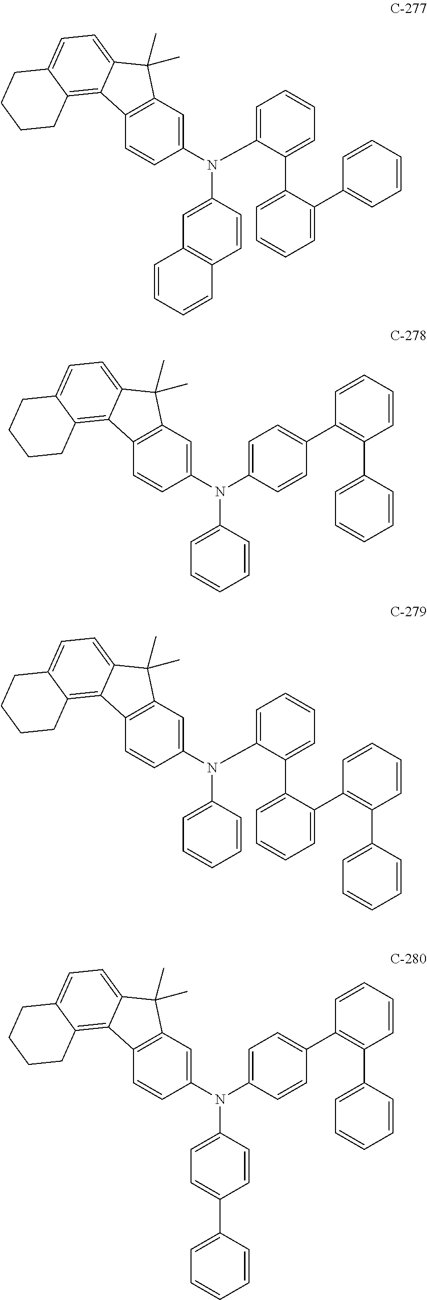 [SMILES: CC1(C)c2cc(N(c3ccc4ccccc4c3)c3ccccc3-c3ccccc3-c3ccccc3)ccc2-c2c1ccc1c2CCCC1 ; CC1(C)c2cc(N(c3ccccc3)c3ccc(-c4ccccc4-c4ccccc4)cc3)ccc2-c2c1ccc1c2CCCC1 ; CC1(C)c2cc(N(c3ccccc3)c3ccccc3-c3ccccc3-c3ccccc3-c3ccccc3)ccc2-c2c1ccc1c2CCCC1 ; CC1(C)c2cc(N(c3ccc(-c4ccccc4)cc3)c3ccc(-c4ccccc4-c4ccccc4)cc3)ccc2-c2c1ccc1c2CCCC1]

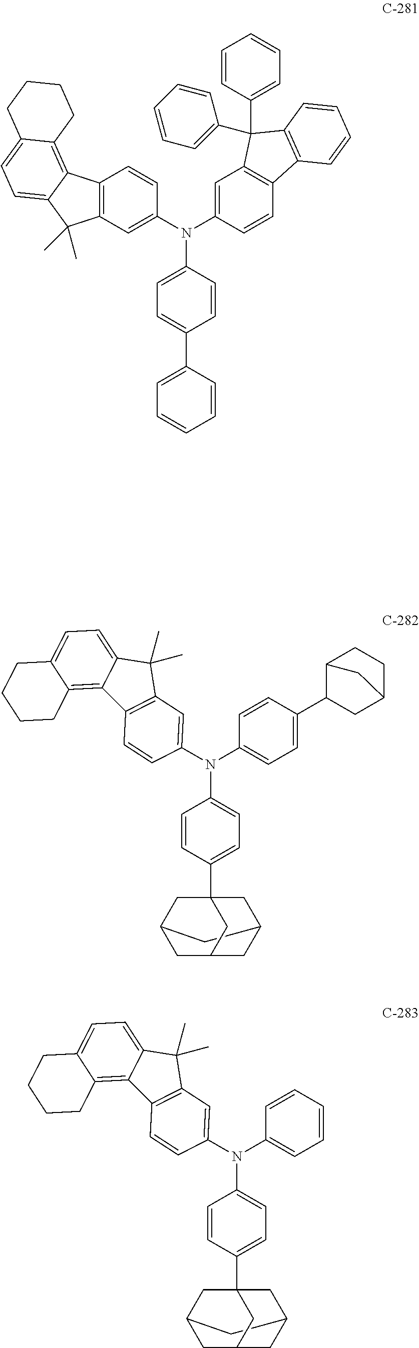 [SMILES: CC1(C)c2cc(N(c3ccc(-c4ccccc4)cc3)c3ccc4c(c3)C(c3ccccc3)(c3ccccc3)c3ccccc3-4)ccc2-c2c1ccc1c2CCCC1 ; CC1(C)c2cc(N(c3ccc(C4CC5CCC4C5)cc3)c3ccc(C45CC6CC(CC(C6)C4)C5)cc3)ccc2-c2c1ccc1c2CCCC1 ; CC1(C)c2cc(N(c3ccccc3)c3ccc(C45CC6CC(CC(C6)C4)C5)cc3)ccc2-c2c1ccc1c2CCCC1]

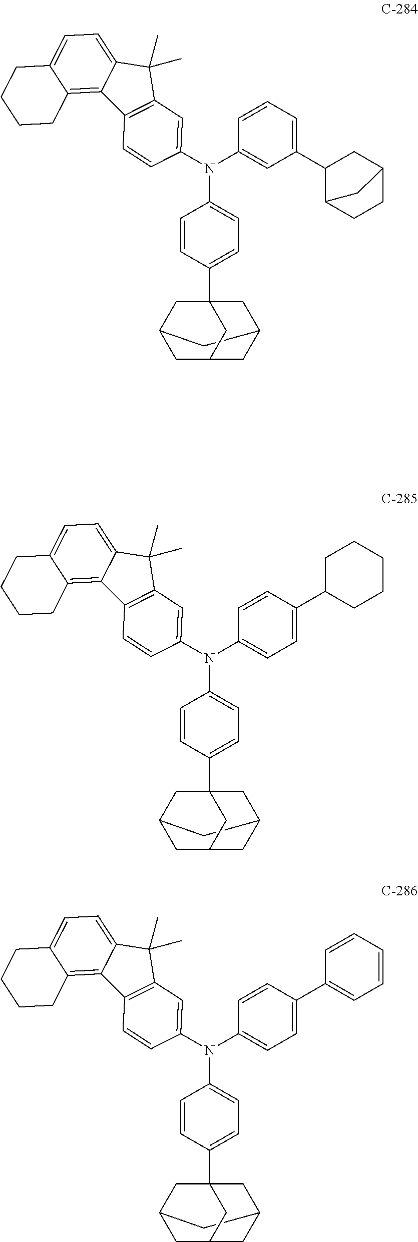 [SMILES: CC1(C)c2cc(N(c3ccc(C45CC6CC(CC(C6)C4)C5)cc3)c3cccc(C4CC5CCC4C5)c3)ccc2-c2c1ccc1c2CCCC1 ; CC1(C)c2cc(N(c3ccc(C4CCCCC4)cc3)c3ccc(C45CC6CC(CC(C6)C4)C5)cc3)ccc2-c2c1ccc1c2CCCC1 ; CC1(C)c2cc(N(c3ccc(-c4ccccc4)cc3)c3ccc(C45CC6CC(CC(C6)C4)C5)cc3)ccc2-c2c1ccc1c2CCCC1]

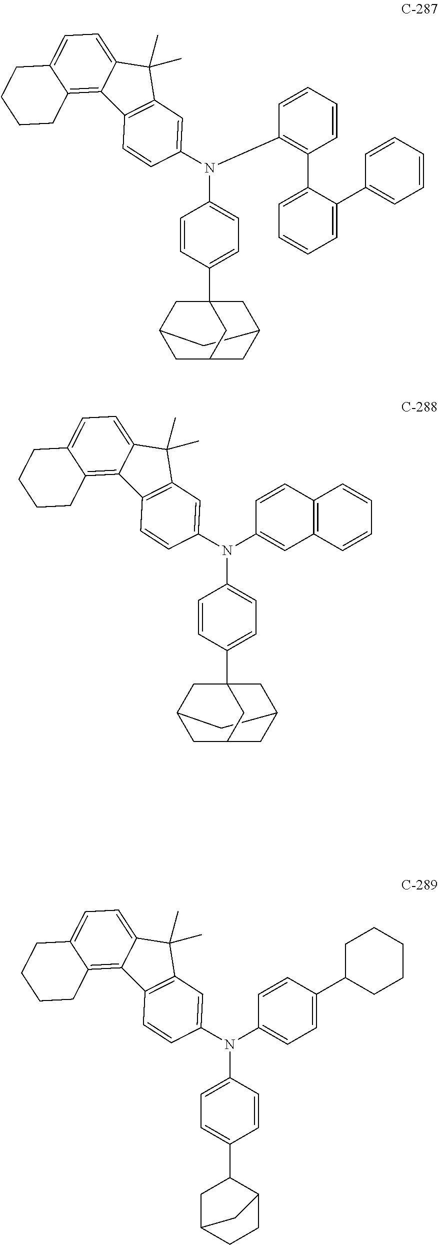 [SMILES: CC1(C)c2cc(N(c3ccc(C45CC6CC(CC(C6)C4)C5)cc3)c3ccccc3-c3ccccc3-c3ccccc3)ccc2-c2c1ccc1c2CCCC1 ; CC1(C)c2cc(N(c3ccc(C45CC6CC(CC(C6)C4)C5)cc3)c3ccc4ccccc4c3)ccc2-c2c1ccc1c2CCCC1 ; CC1(C)c2cc(N(c3ccc(C4CCCCC4)cc3)c3ccc(C4CC5CCC4C5)cc3)ccc2-c2c1ccc1c2CCCC1]

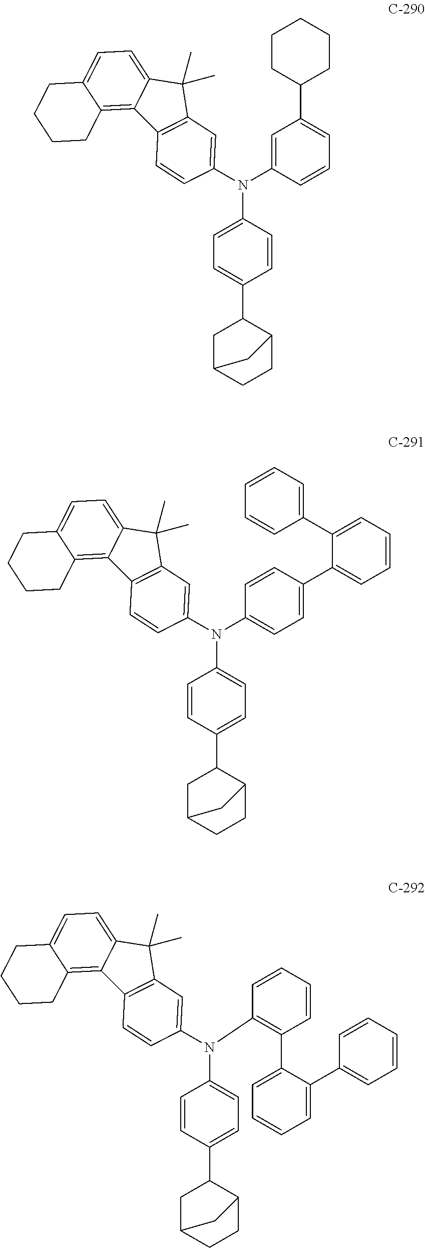 [SMILES: CC1(C)c2cc(N(c3ccc(C4CC5CCC4C5)cc3)c3cccc(C4CCCCC4)c3)ccc2-c2c1ccc1c2CCCC1 ; CC1(C)c2cc(N(c3ccc(-c4ccccc4-c4ccccc4)cc3)c3ccc(C4CC5CCC4C5)cc3)ccc2-c2c1ccc1c2CCCC1 ; CC1(C)c2cc(N(c3ccc(C4CC5CCC4C5)cc3)c3ccccc3-c3ccccc3-c3ccccc3)ccc2-c2c1ccc1c2CCCC1]

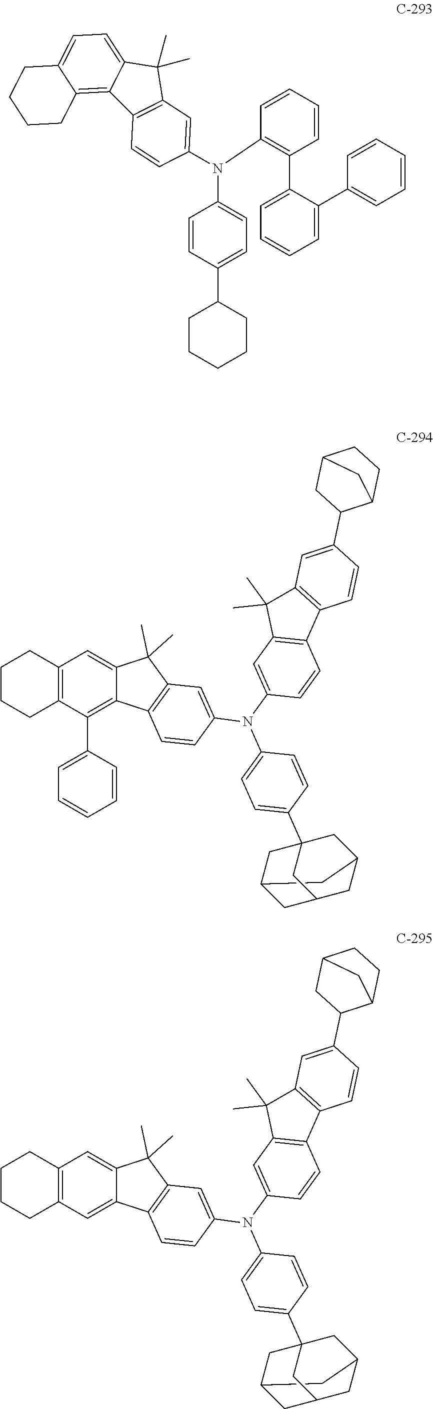 [SMILES: CC1(C)c2cc(N(c3ccc(C4CCCCC4)cc3)c3ccccc3-c3ccccc3-c3ccccc3)ccc2-c2c1ccc1c2CCCC1 ; CC1(C)c2cc(C3CC4CCC3C4)ccc2-c2ccc(N(c3ccc(C45CC6CC(CC(C6)C4)C5)cc3)c3ccc4c(c3)C(C)(C)c3cc5c(c(-c6ccccc6)c3-4)CCCC5)cc21 ; CC1(C)c2cc(C3CC4CCC3C4)ccc2-c2ccc(N(c3ccc(C45CC6CC(CC(C6)C4)C5)cc3)c3ccc4c(c3)C(C)(C)c3cc5c(cc3-4)CCCC5)cc21]

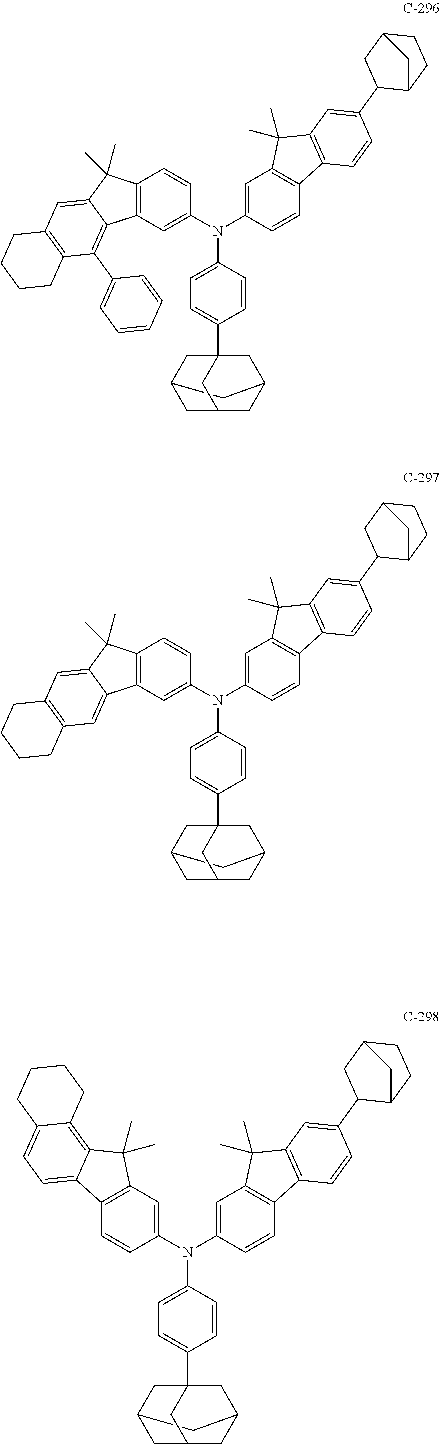 [SMILES: CC1(C)c2cc(C3CC4CCC3C4)ccc2-c2ccc(N(c3ccc(C45CC6CC(CC(C6)C4)C5)cc3)c3ccc4c(c3)-c3c(cc5c(c3-c3ccccc3)CCCC5)C4(C)C)cc21 ; CC1(C)c2cc(C3CC4CCC3C4)ccc2-c2ccc(N(c3ccc(C45CC6CC(CC(C6)C4)C5)cc3)c3ccc4c(c3)-c3cc5c(cc3C4(C)C)CCCC5)cc21 ; CC1(C)c2cc(C3CC4CCC3C4)ccc2-c2ccc(N(c3ccc(C45CC6CC(CC(C6)C4)C5)cc3)c3ccc4c(c3)C(C)(C)c3c-4ccc4c3CCCC4)cc21]

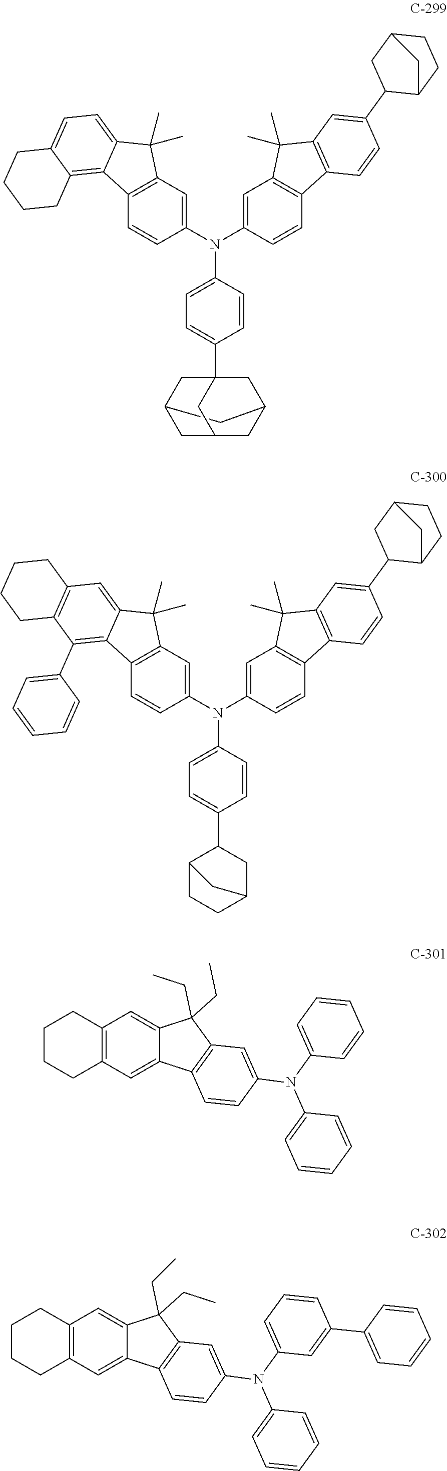 [SMILES: CC1(C)c2cc(C3CC4CCC3C4)ccc2-c2ccc(N(c3ccc(C45CC6CC(CC(C6)C4)C5)cc3)c3ccc4c(c3)C(C)(C)c3ccc5c(c3-4)CCCC5)cc21 ; CC1(C)c2cc(C3CC4CCC3C4)ccc2-c2ccc(N(c3ccc(C4CC5CCC4C5)cc3)c3ccc4c(c3)C(C)(C)c3cc5c(c(-c6ccccc6)c3-4)CCCC5)cc21 ; CCC1(CC)c2cc(N(c3ccccc3)c3ccccc3)ccc2-c2cc3c(cc21)CCCC3 ; CCC1(CC)c2cc(N(c3ccccc3)c3cccc(-c4ccccc4)c3)ccc2-c2cc3c(cc21)CCCC3]

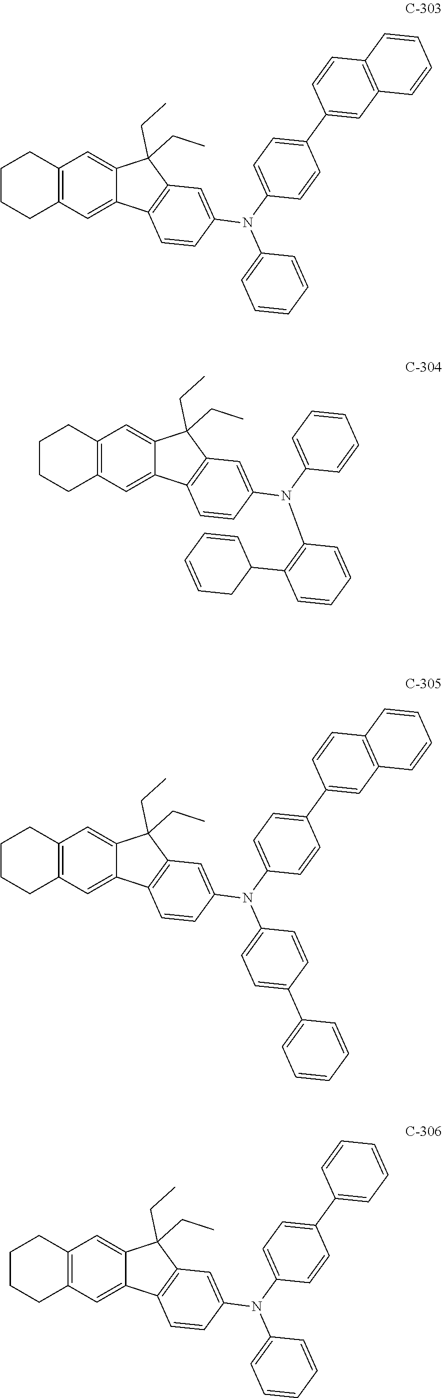 [SMILES: CCC1(CC)c2cc(N(c3ccccc3)c3ccc(-c4ccc5ccccc5c4)cc3)ccc2-c2cc3c(cc21)CCCC3 ; CCC1(CC)c2cc(N(c3ccccc3)c3ccccc3C3C=CC=CC3)ccc2-c2cc3c(cc21)CCCC3 ; CCC1(CC)c2cc(N(c3ccc(-c4ccccc4)cc3)c3ccc(-c4ccc5ccccc5c4)cc3)ccc2-c2cc3c(cc21)CCCC3 ; CCC1(CC)c2cc(N(c3ccccc3)c3ccc(-c4ccccc4)cc3)ccc2-c2cc3c(cc21)CCCC3]

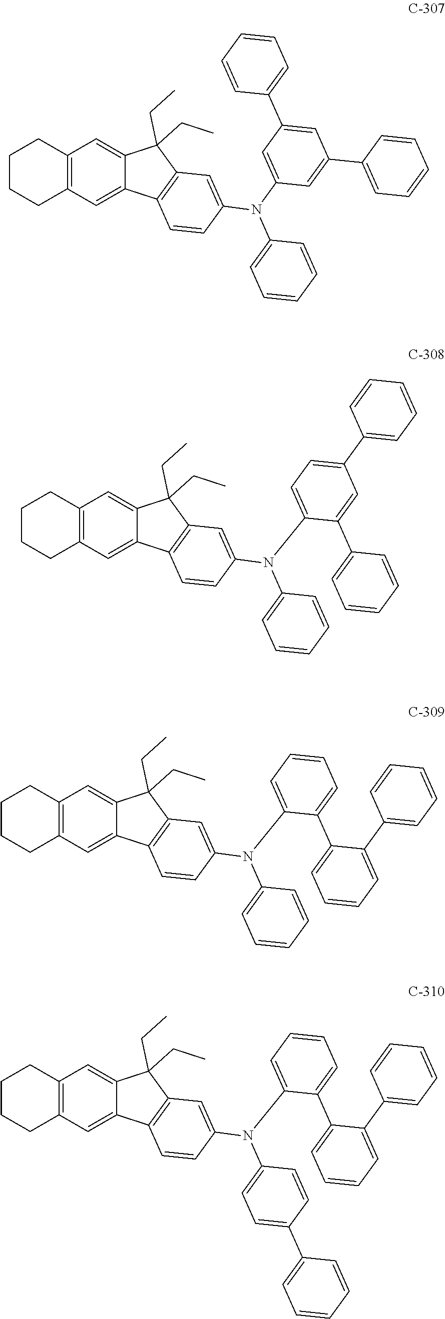 [SMILES: CCC1(CC)c2cc(N(c3ccccc3)c3cc(-c4ccccc4)cc(-c4ccccc4)c3)ccc2-c2cc3c(cc21)CCCC3 ; CCC1(CC)c2cc(N(c3ccccc3)c3ccc(-c4ccccc4)cc3-c3ccccc3)ccc2-c2cc3c(cc21)CCCC3 ; CCC1(CC)c2cc(N(c3ccccc3)c3ccccc3-c3ccccc3-c3ccccc3)ccc2-c2cc3c(cc21)CCCC3 ; CCC1(CC)c2cc(N(c3ccc(-c4ccccc4)cc3)c3ccccc3-c3ccccc3-c3ccccc3)ccc2-c2cc3c(cc21)CCCC3]

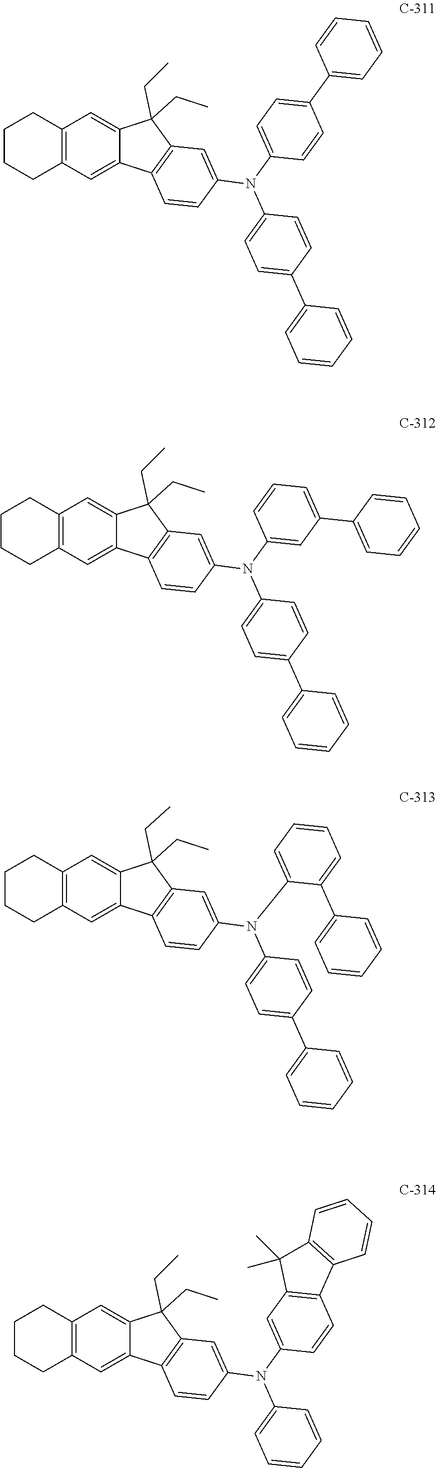 [SMILES: CCC1(CC)c2cc(N(c3ccc(-c4ccccc4)cc3)c3ccc(-c4ccccc4)cc3)ccc2-c2cc3c(cc21)CCCC3 ; CCC1(CC)c2cc(N(c3ccc(-c4ccccc4)cc3)c3cccc(-c4ccccc4)c3)ccc2-c2cc3c(cc21)CCCC3 ; CCC1(CC)c2cc(N(c3ccc(-c4ccccc4)cc3)c3ccccc3-c3ccccc3)ccc2-c2cc3c(cc21)CCCC3 ; CCC1(CC)c2cc(N(c3ccccc3)c3ccc4c(c3)C(C)(C)c3ccccc3-4)ccc2-c2cc3c(cc21)CCCC3]

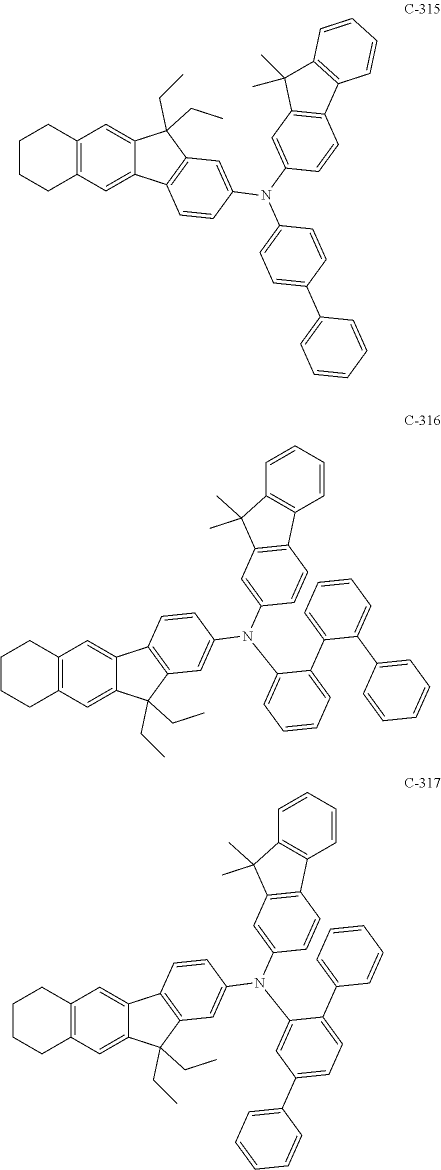 [SMILES: CCC1(CC)c2cc(N(c3ccc(-c4ccccc4)cc3)c3ccc4c(c3)C(C)(C)c3ccccc3-4)ccc2-c2cc3c(cc21)CCCC3 ; CCC1(CC)c2cc(N(c3ccc4c(c3)C(C)(C)c3ccccc3-4)c3ccccc3-c3ccccc3-c3ccccc3)ccc2-c2cc3c(cc21)CCCC3 ; CCC1(CC)c2cc(N(c3ccc4c(c3)C(C)(C)c3ccccc3-4)c3cc(-c4ccccc4)ccc3-c3ccccc3)ccc2-c2cc3c(cc21)CCCC3]

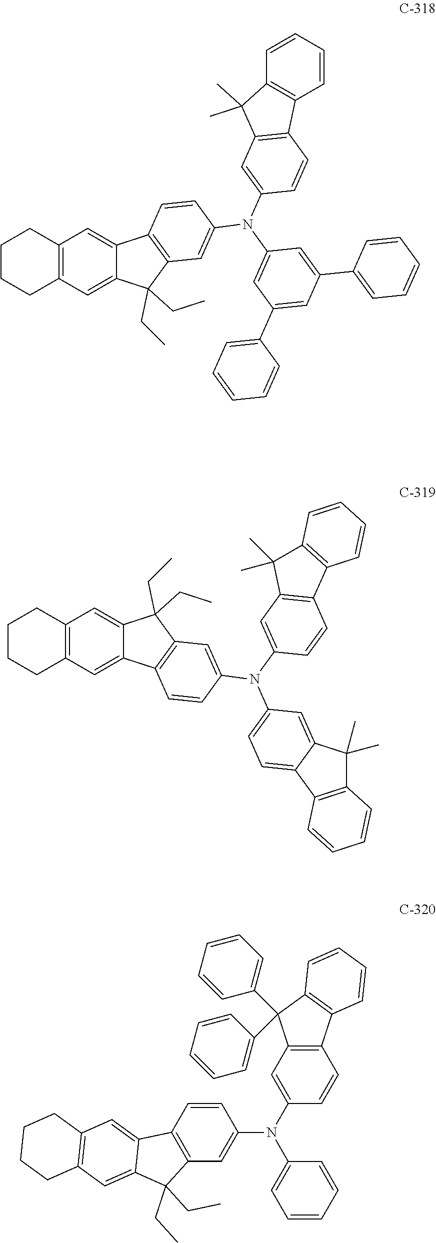 [SMILES: CCC1(CC)c2cc(N(c3cc(-c4ccccc4)cc(-c4ccccc4)c3)c3ccc4c(c3)C(C)(C)c3ccccc3-4)ccc2-c2cc3c(cc21)CCCC3 ; CCC1(CC)c2cc(N(c3ccc4c(c3)C(C)(C)c3ccccc3-4)c3ccc4c(c3)C(C)(C)c3ccccc3-4)ccc2-c2cc3c(cc21)CCCC3 ; CCC1(CC)c2cc(N(c3ccccc3)c3ccc4c(c3)C(c3ccccc3)(c3ccccc3)c3ccccc3-4)ccc2-c2cc3c(cc21)CCCC3]

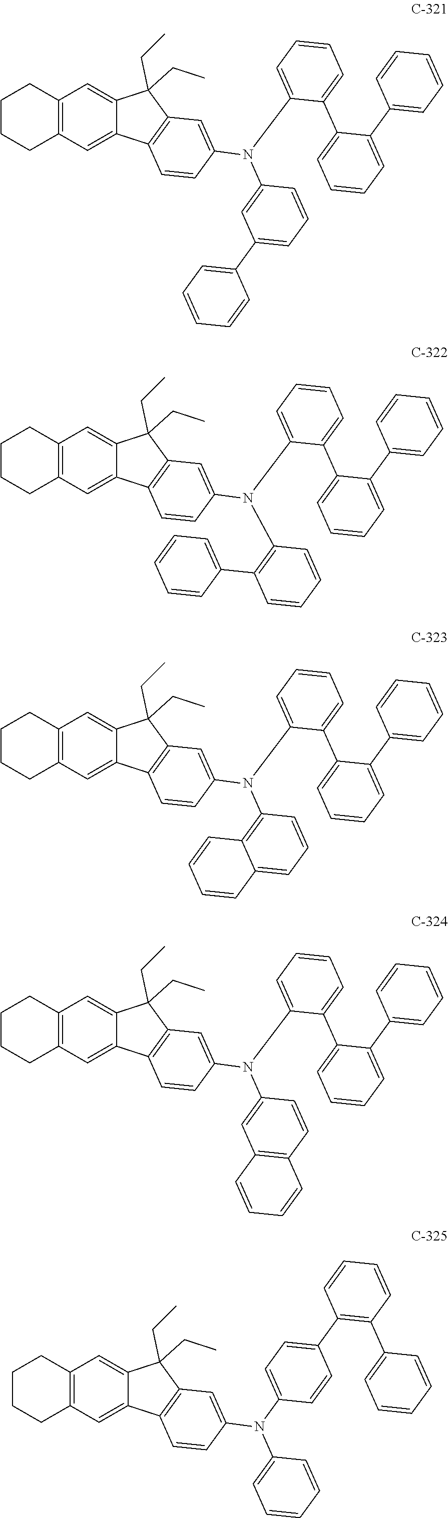 [SMILES: CCC1(CC)c2cc(N(c3cccc(-c4ccccc4)c3)c3ccccc3-c3ccccc3-c3ccccc3)ccc2-c2cc3c(cc21)CCCC3 ; CCC1(CC)c2cc(N(c3ccccc3-c3ccccc3)c3ccccc3-c3ccccc3-c3ccccc3)ccc2-c2cc3c(cc21)CCCC3 ; CCC1(CC)c2cc(N(c3ccccc3-c3ccccc3-c3ccccc3)c3cccc4ccccc34)ccc2-c2cc3c(cc21)CCCC3 ; CCC1(CC)c2cc(N(c3ccc4ccccc4c3)c3ccccc3-c3ccccc3-c3ccccc3)ccc2-c2cc3c(cc21)CCCC3 ; CCC1(CC)c2cc(N(c3ccccc3)c3ccc(-c4ccccc4-c4ccccc4)cc3)ccc2-c2cc3c(cc21)CCCC3]

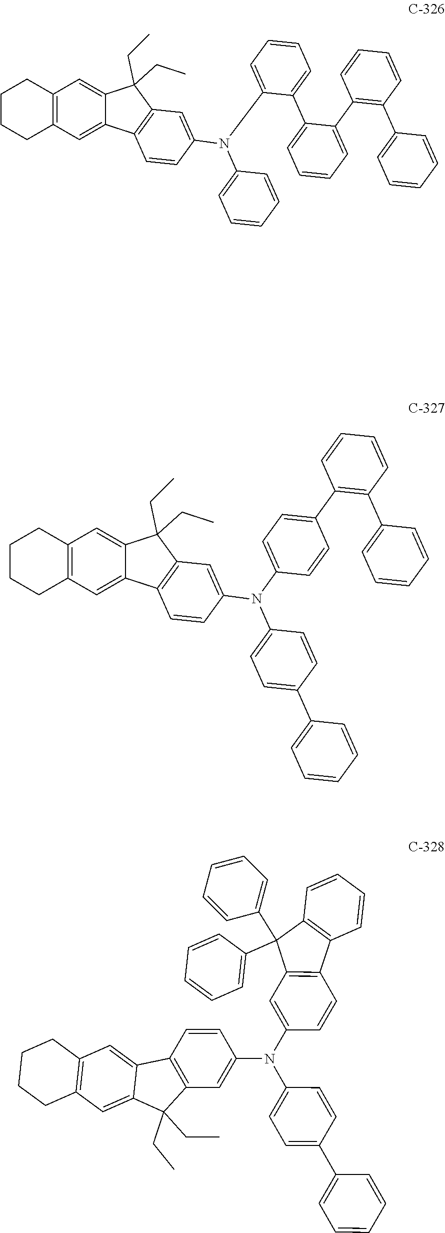 [SMILES: CCC1(CC)c2cc(N(c3ccccc3)c3ccccc3-c3ccccc3-c3ccccc3-c3ccccc3)ccc2-c2cc3c(cc21)CCCC3 ; CCC1(CC)c2cc(N(c3ccc(-c4ccccc4)cc3)c3ccc(-c4ccccc4-c4ccccc4)cc3)ccc2-c2cc3c(cc21)CCCC3 ; CCC1(CC)c2cc(N(c3ccc(-c4ccccc4)cc3)c3ccc4c(c3)C(c3ccccc3)(c3ccccc3)c3ccccc3-4)ccc2-c2cc3c(cc21)CCCC3]

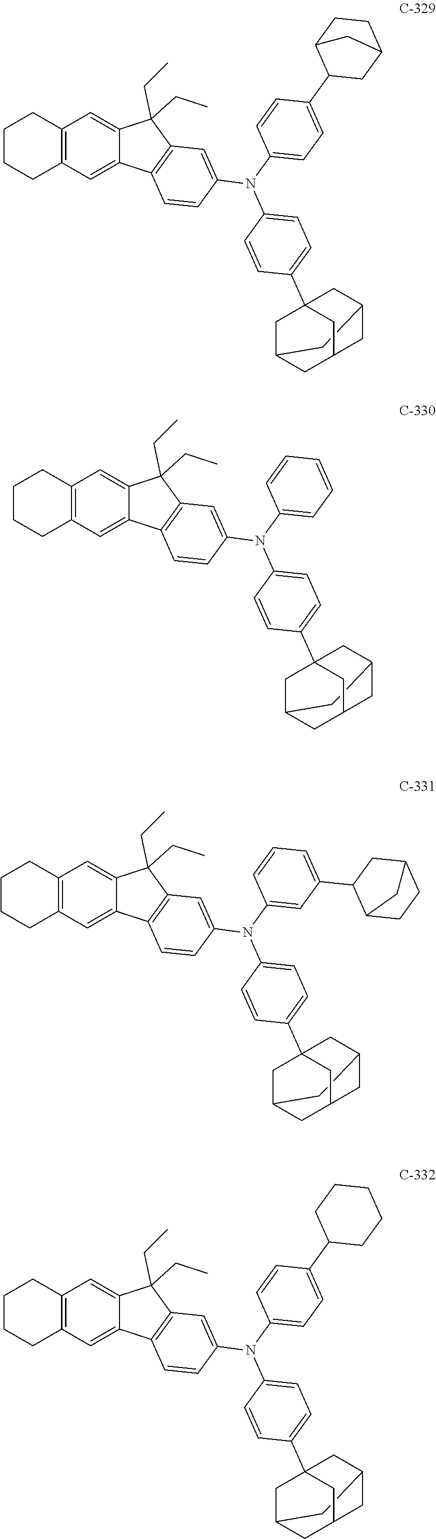 [SMILES: CCC1(CC)c2cc(N(c3ccc(C4CC5CCC4C5)cc3)c3ccc(C45CC6CC(CC(C6)C4)C5)cc3)ccc2-c2cc3c(cc21)CCCC3 ; CCC1(CC)c2cc(N(c3ccccc3)c3ccc(C45CC6CC(CC(C6)C4)C5)cc3)ccc2-c2cc3c(cc21)CCCC3 ; CCC1(CC)c2cc(N(c3ccc(C45CC6CC(CC(C6)C4)C5)cc3)c3cccc(C4CC5CCC4C5)c3)ccc2-c2cc3c(cc21)CCCC3 ; CCC1(CC)c2cc(N(c3ccc(C4CCCCC4)cc3)c3ccc(C45CC6CC(CC(C6)C4)C5)cc3)ccc2-c2cc3c(cc21)CCCC3]

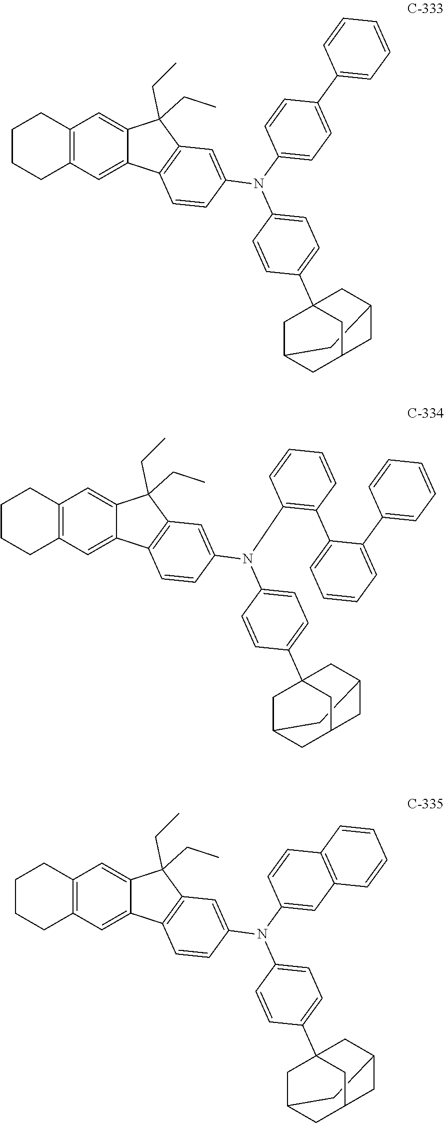 [SMILES: CCC1(CC)c2cc(N(c3ccc(-c4ccccc4)cc3)c3ccc(C45CC6CC(CC(C6)C4)C5)cc3)ccc2-c2cc3c(cc21)CCCC3 ; CCC1(CC)c2cc(N(c3ccc(C45CC6CC(CC(C6)C4)C5)cc3)c3ccccc3-c3ccccc3-c3ccccc3)ccc2-c2cc3c(cc21)CCCC3 ; CCC1(CC)c2cc(N(c3ccc(C45CC6CC(CC(C6)C4)C5)cc3)c3ccc4ccccc4c3)ccc2-c2cc3c(cc21)CCCC3]

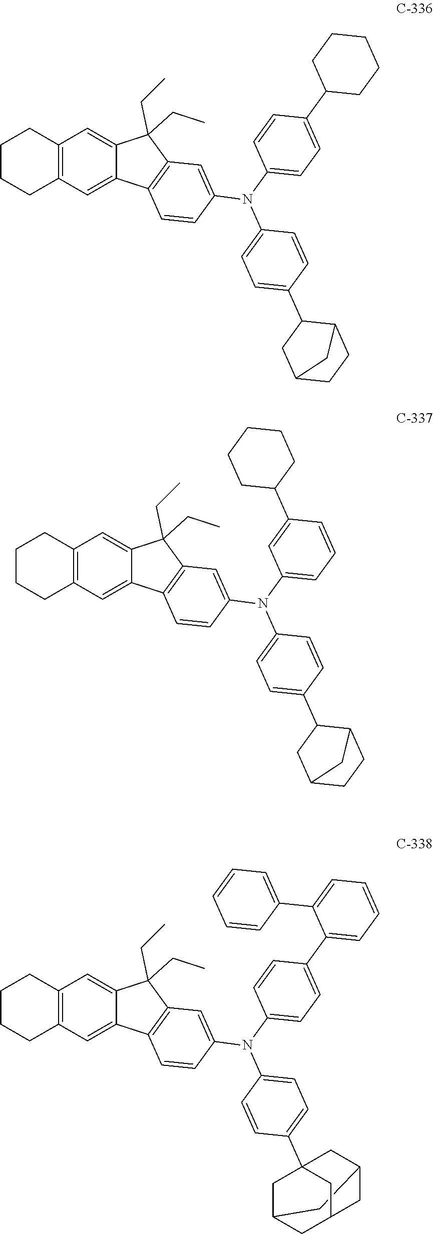 [SMILES: CCC1(CC)c2cc(N(c3ccc(C4CCCCC4)cc3)c3ccc(C4CC5CCC4C5)cc3)ccc2-c2cc3c(cc21)CCCC3 ; CCC1(CC)c2cc(N(c3ccc(C4CC5CCC4C5)cc3)c3cccc(C4CCCCC4)c3)ccc2-c2cc3c(cc21)CCCC3 ; CCC1(CC)c2cc(N(c3ccc(-c4ccccc4-c4ccccc4)cc3)c3ccc(C45CC6CC(CC(C6)C4)C5)cc3)ccc2-c2cc3c(cc21)CCCC3]

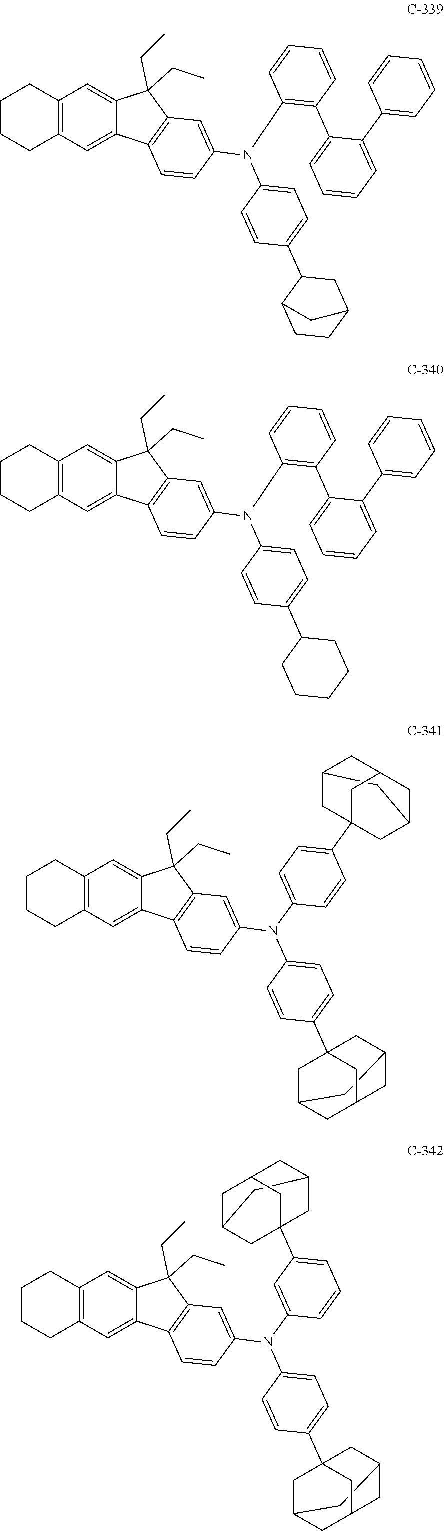 [SMILES: CCC1(CC)c2cc(N(c3ccc(C4CC5CCC4C5)cc3)c3ccccc3-c3ccccc3-c3ccccc3)ccc2-c2cc3c(cc21)CCCC3 ; CCC1(CC)c2cc(N(c3ccc(C4CCCCC4)cc3)c3ccccc3-c3ccccc3-c3ccccc3)ccc2-c2cc3c(cc21)CCCC3 ; CCC1(CC)c2cc(N(c3ccc(C45CC6CC(CC(C6)C4)C5)cc3)c3ccc(C45CC6CC(CC(C6)C4)C5)cc3)ccc2-c2cc3c(cc21)CCCC3 ; CCC1(CC)c2cc(N(c3ccc(C45CC6CC(CC(C6)C4)C5)cc3)c3cccc(C45CC6CC(CC(C6)C4)C5)c3)ccc2-c2cc3c(cc21)CCCC3]

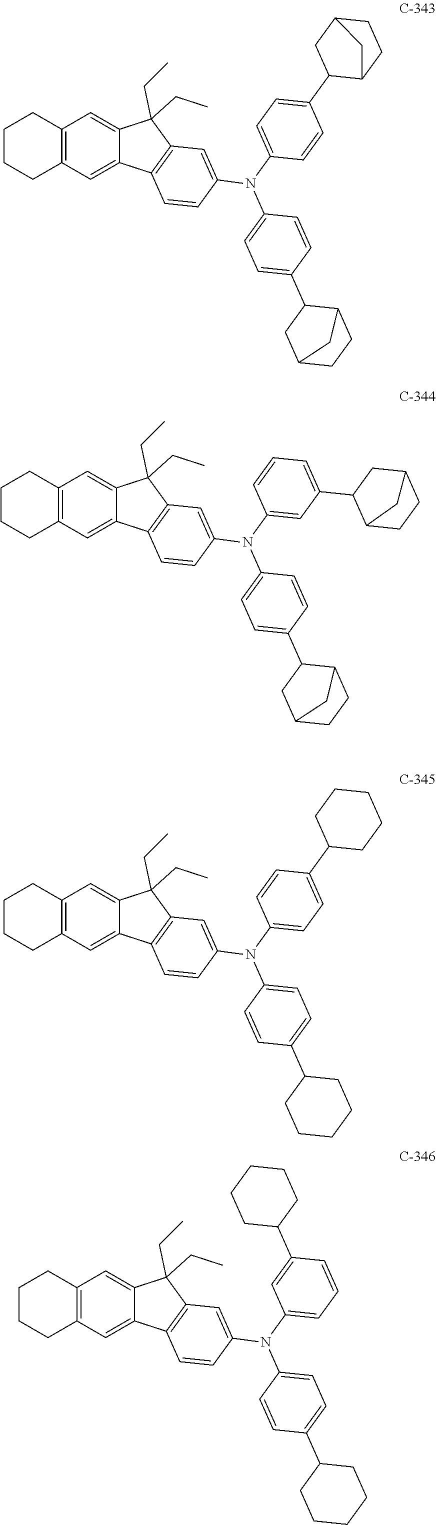

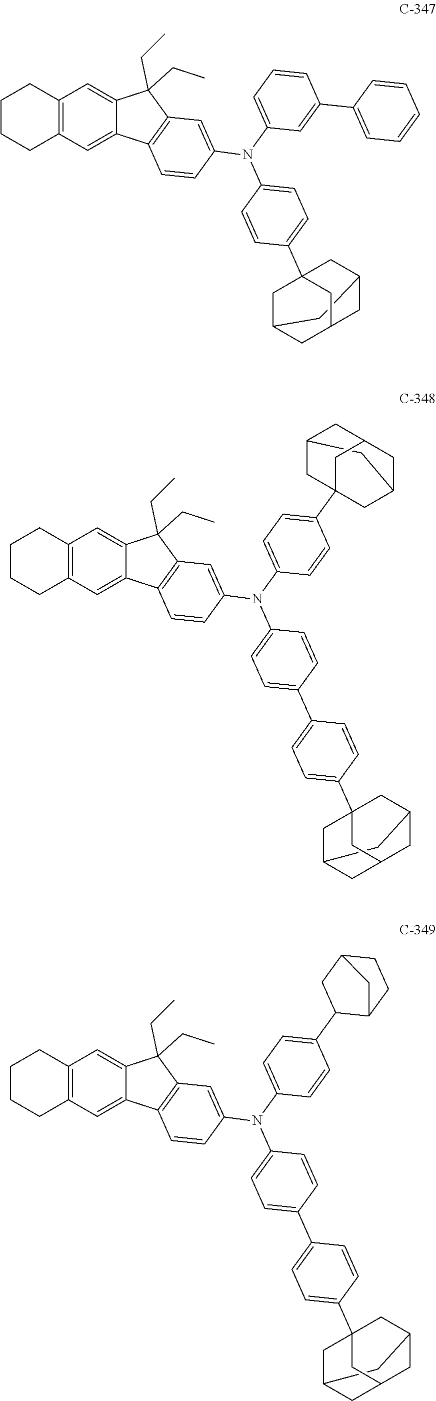 [SMILES: CCC1(CC)c2cc(N(c3ccc(C45CC6CC(CC(C6)C4)C5)cc3)c3cccc(-c4ccccc4)c3)ccc2-c2cc3c(cc21)CCCC3 ; CCC1(CC)c2cc(N(c3ccc(-c4ccc(C56CC7CC(CC(C7)C5)C6)cc4)cc3)c3ccc(C45CC6CC(CC(C6)C4)C5)cc3)ccc2-c2cc3c(cc21)CCCC3 ; CCC1(CC)c2cc(N(c3ccc(-c4ccc(C56CC7CC(CC(C7)C5)C6)cc4)cc3)c3ccc(C4CC5CCC4C5)cc3)ccc2-c2cc3c(cc21)CCCC3]

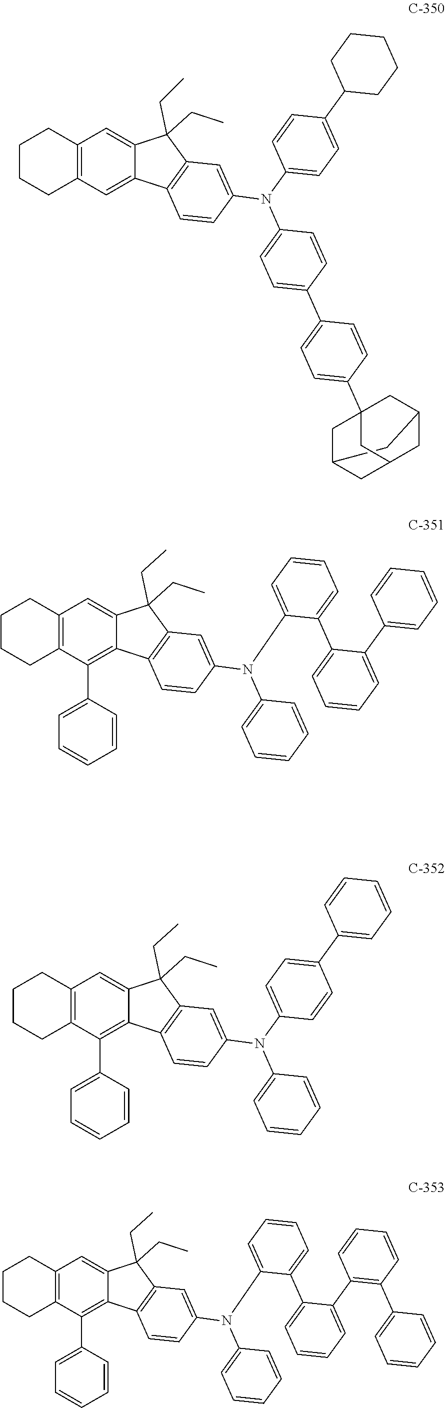 [SMILES: CCC1(CC)c2cc(N(c3ccc(-c4ccc(C56CC7CC(CC(C7)C5)C6)cc4)cc3)c3ccc(C4CCCCC4)cc3)ccc2-c2cc3c(cc21)CCCC3 ; CCC1(CC)c2cc(N(c3ccccc3)c3ccccc3-c3ccccc3-c3ccccc3)ccc2-c2c1cc1c(c2-c2ccccc2)CCCC1 ; CCC1(CC)c2cc(N(c3ccccc3)c3ccc(-c4ccccc4)cc3)ccc2-c2c1cc1c(c2-c2ccccc2)CCCC1 ; CCC1(CC)c2cc(N(c3ccccc3)c3ccccc3-c3ccccc3-c3ccccc3-c3ccccc3)ccc2-c2c1cc1c(c2-c2ccccc2)CCCC1]

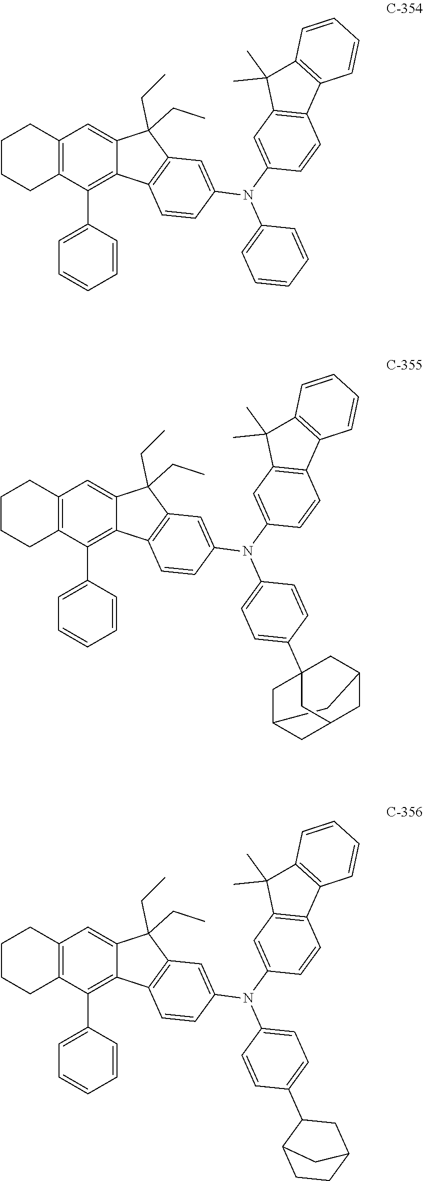 [SMILES: CCC1(CC)c2cc(N(c3ccccc3)c3ccc4c(c3)C(C)(C)c3ccccc3-4)ccc2-c2c1cc1c(c2-c2ccccc2)CCCC1 ; CCC1(CC)c2cc(N(c3ccc(C45CC6CC(CC(C6)C4)C5)cc3)c3ccc4c(c3)C(C)(C)c3ccccc3-4)ccc2-c2c1cc1c(c2-c2ccccc2)CCCC1 ; CCC1(CC)c2cc(N(c3ccc(C4CC5CCC4C5)cc3)c3ccc4c(c3)C(C)(C)c3ccccc3-4)ccc2-c2c1cc1c(c2-c2ccccc2)CCCC1]

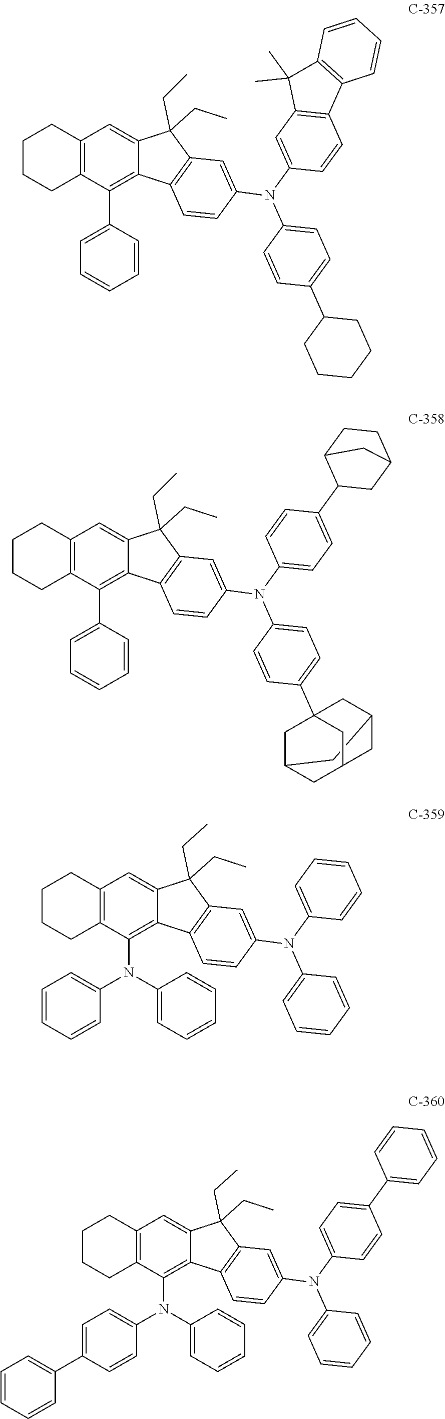 [SMILES: CCC1(CC)c2cc(N(c3ccc(C4CCCCC4)cc3)c3ccc4c(c3)C(C)(C)c3ccccc3-4)ccc2-c2c1cc1c(c2-c2ccccc2)CCCC1 ; CCC1(CC)c2cc(N(c3ccc(C4CC5CCC4C5)cc3)c3ccc(C45CC6CC(CC(C6)C4)C5)cc3)ccc2-c2c1cc1c(c2-c2ccccc2)CCCC1 ; CCC1(CC)c2cc(N(c3ccccc3)c3ccccc3)ccc2-c2c1cc1c(c2N(c2ccccc2)c2ccccc2)CCCC1 ; CCC1(CC)c2cc(N(c3ccccc3)c3ccc(-c4ccccc4)cc3)ccc2-c2c1cc1c(c2N(c2ccccc2)c2ccc(-c3ccccc3)cc2)CCCC1]

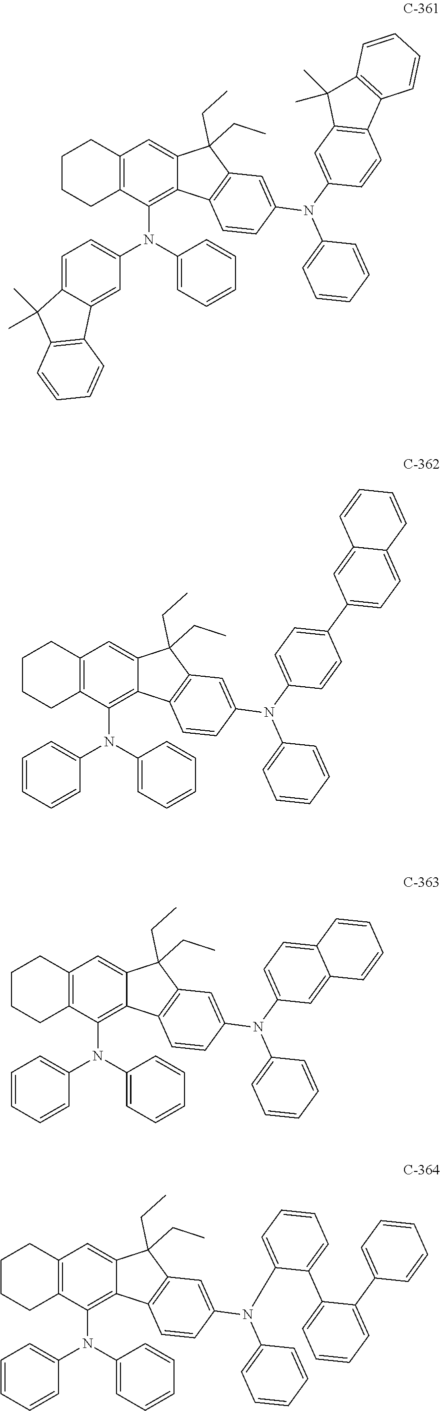 [SMILES: CCC1(CC)c2cc(N(c3ccccc3)c3ccc4c(c3)C(C)(C)c3ccccc3-4)ccc2-c2c1cc1c(c2N(c2ccccc2)c2ccc3c(c2)-c2ccccc2C3(C)C)CCCC1 ; CCC1(CC)c2cc(N(c3ccccc3)c3ccc(-c4ccc5ccccc5c4)cc3)ccc2-c2c1cc1c(c2N(c2ccccc2)c2ccccc2)CCCC1 ; CCC1(CC)c2cc(N(c3ccccc3)c3ccc4ccccc4c3)ccc2-c2c1cc1c(c2N(c2ccccc2)c2ccccc2)CCCC1 ; CCC1(CC)c2cc(N(c3ccccc3)c3ccccc3-c3ccccc3-c3ccccc3)ccc2-c2c1cc1c(c2N(c2ccccc2)c2ccccc2)CCCC1]

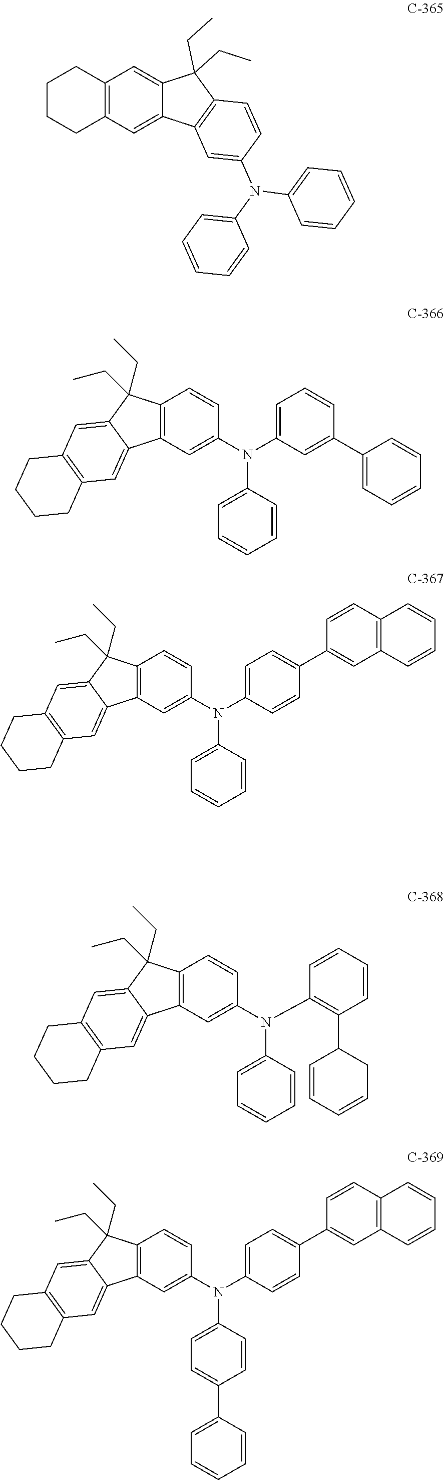 [SMILES: CCC1(CC)c2ccc(N(c3ccccc3)c3ccccc3)cc2-c2cc3c(cc21)CCCC3 ; CCC1(CC)c2ccc(N(c3ccccc3)c3cccc(-c4ccccc4)c3)cc2-c2cc3c(cc21)CCCC3 ; CCC1(CC)c2ccc(N(c3ccccc3)c3ccc(-c4ccc5ccccc5c4)cc3)cc2-c2cc3c(cc21)CCCC3 ; CCC1(CC)c2ccc(N(c3ccccc3)c3ccccc3C3C=CC=CC3)cc2-c2cc3c(cc21)CCCC3 ; CCC1(CC)c2ccc(N(c3ccc(-c4ccccc4)cc3)c3ccc(-c4ccc5ccccc5c4)cc3)cc2-c2cc3c(cc21)CCCC3]

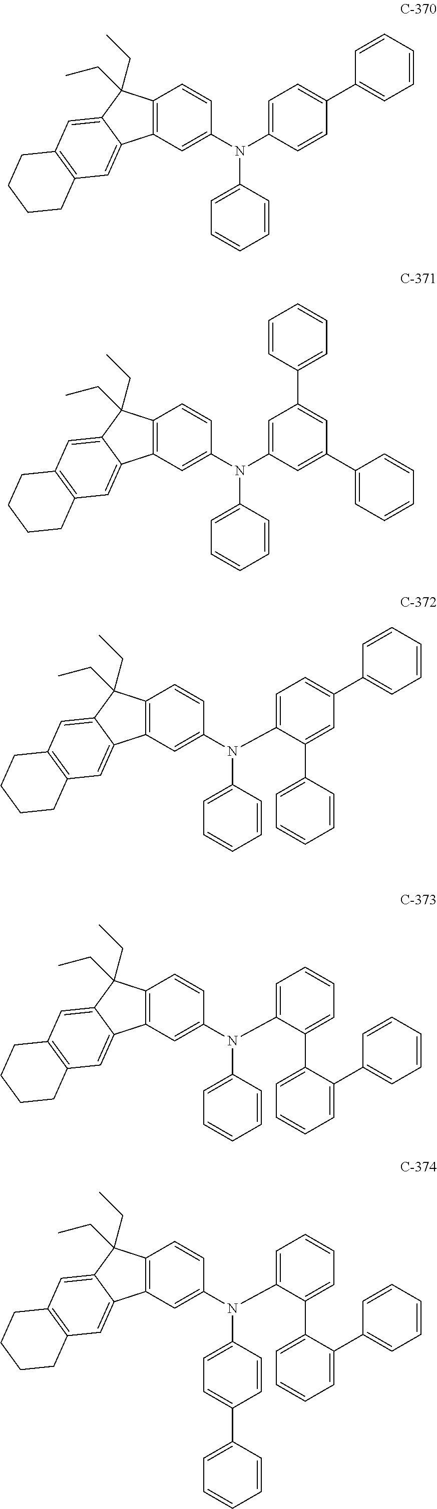 [SMILES: CCC1(CC)c2ccc(N(c3ccccc3)c3ccc(-c4ccccc4)cc3)cc2-c2cc3c(cc21)CCCC3 ; CCC1(CC)c2ccc(N(c3ccccc3)c3cc(-c4ccccc4)cc(-c4ccccc4)c3)cc2-c2cc3c(cc21)CCCC3 ; CCC1(CC)c2ccc(N(c3ccccc3)c3ccc(-c4ccccc4)cc3-c3ccccc3)cc2-c2cc3c(cc21)CCCC3 ; CCC1(CC)c2ccc(N(c3ccccc3)c3ccccc3-c3ccccc3-c3ccccc3)cc2-c2cc3c(cc21)CCCC3 ; CCC1(CC)c2ccc(N(c3ccc(-c4ccccc4)cc3)c3ccccc3-c3ccccc3-c3ccccc3)cc2-c2cc3c(cc21)CCCC3]

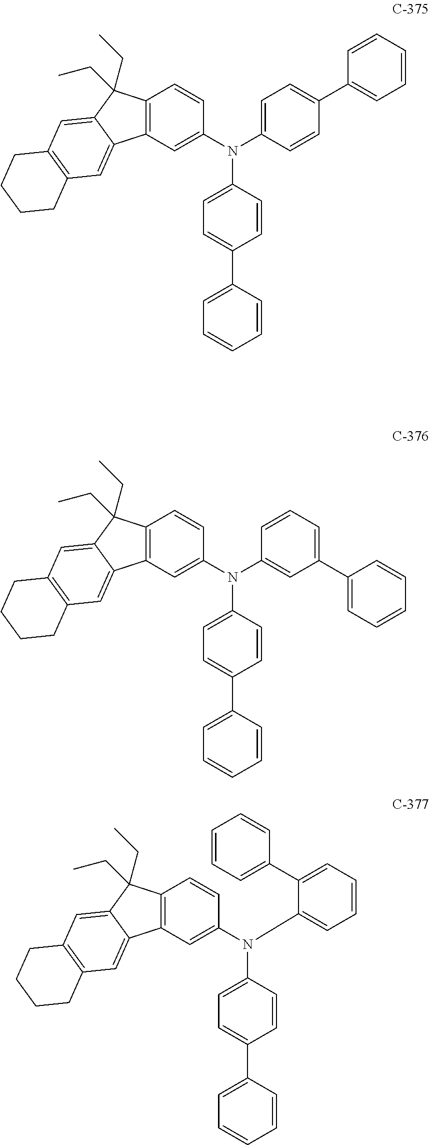

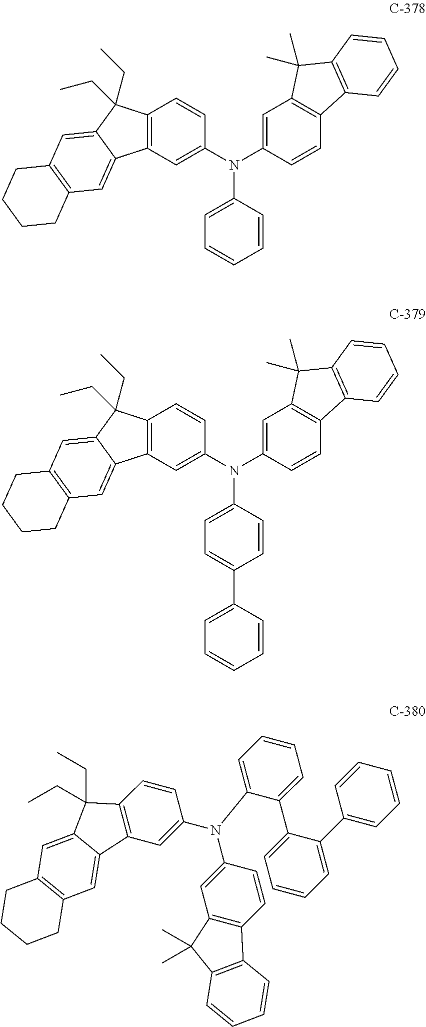 [SMILES: CCC1(CC)c2ccc(N(c3ccccc3)c3ccc4c(c3)C(C)(C)c3ccccc3-4)cc2-c2cc3c(cc21)CCCC3 ; CCC1(CC)c2ccc(N(c3ccc(-c4ccccc4)cc3)c3ccc4c(c3)C(C)(C)c3ccccc3-4)cc2-c2cc3c(cc21)CCCC3 ; CCC1(CC)c2ccc(N(c3ccc4c(c3)C(C)(C)c3ccccc3-4)c3ccccc3-c3ccccc3-c3ccccc3)cc2-c2cc3c(cc21)CCCC3]

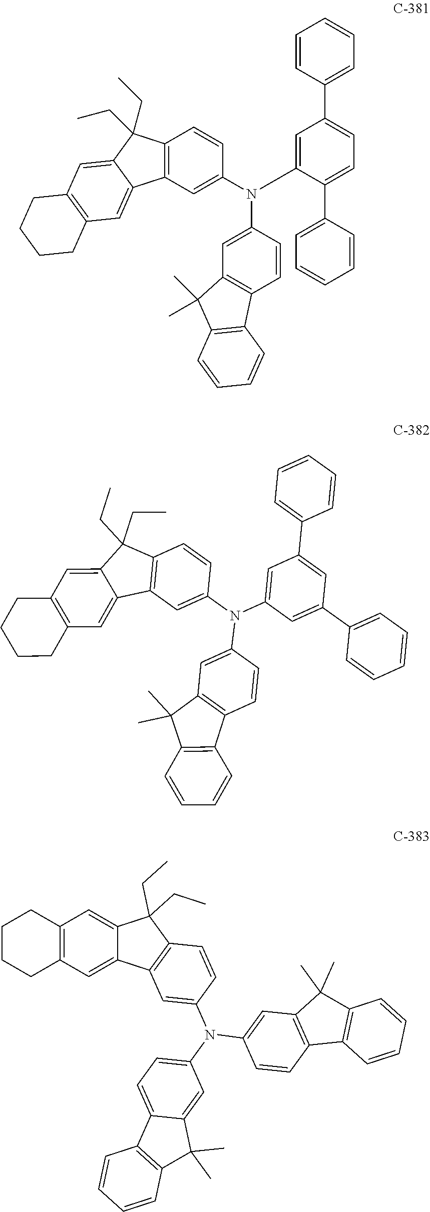 [SMILES: CCC1(CC)c2ccc(N(c3ccc4c(c3)C(C)(C)c3ccccc3-4)c3cc(-c4ccccc4)ccc3-c3ccccc3)cc2-c2cc3c(cc21)CCCC3 ; CCC1(CC)c2ccc(N(c3cc(-c4ccccc4)cc(-c4ccccc4)c3)c3ccc4c(c3)C(C)(C)c3ccccc3-4)cc2-c2cc3c(cc21)CCCC3 ; CCC1(CC)c2ccc(N(c3ccc4c(c3)C(C)(C)c3ccccc3-4)c3ccc4c(c3)C(C)(C)c3ccccc3-4)cc2-c2cc3c(cc21)CCCC3]

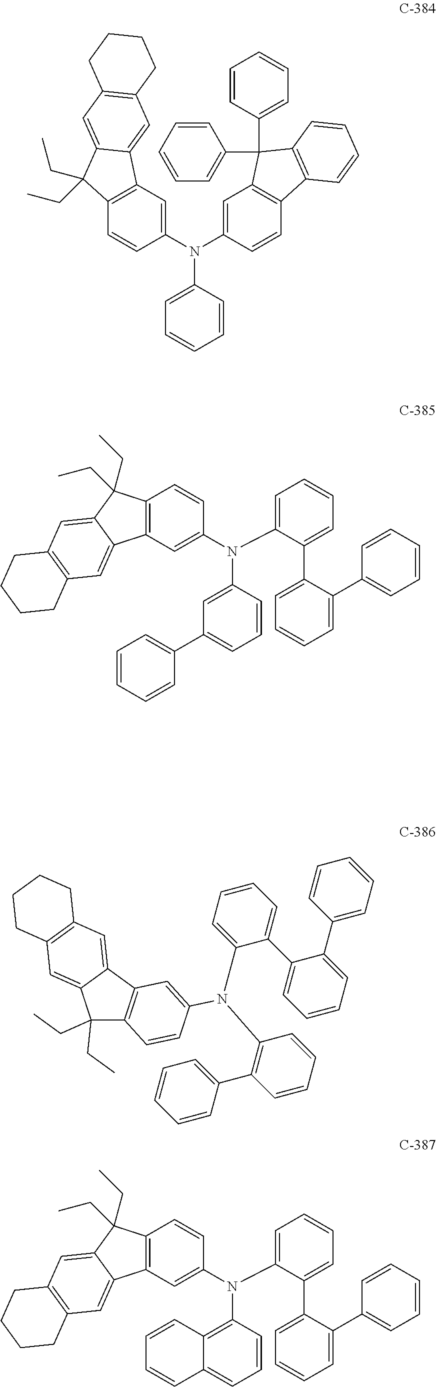 [SMILES: CCC1(CC)c2ccc(N(c3ccccc3)c3ccc4c(c3)C(c3ccccc3)(c3ccccc3)c3ccccc3-4)cc2-c2cc3c(cc21)CCCC3 ; CCC1(CC)c2ccc(N(c3cccc(-c4ccccc4)c3)c3ccccc3-c3ccccc3-c3ccccc3)cc2-c2cc3c(cc21)CCCC3 ; CCC1(CC)c2ccc(N(c3ccccc3-c3ccccc3)c3ccccc3-c3ccccc3-c3ccccc3)cc2-c2cc3c(cc21)CCCC3 ; CCC1(CC)c2ccc(N(c3ccccc3-c3ccccc3-c3ccccc3)c3cccc4ccccc34)cc2-c2cc3c(cc21)CCCC3]

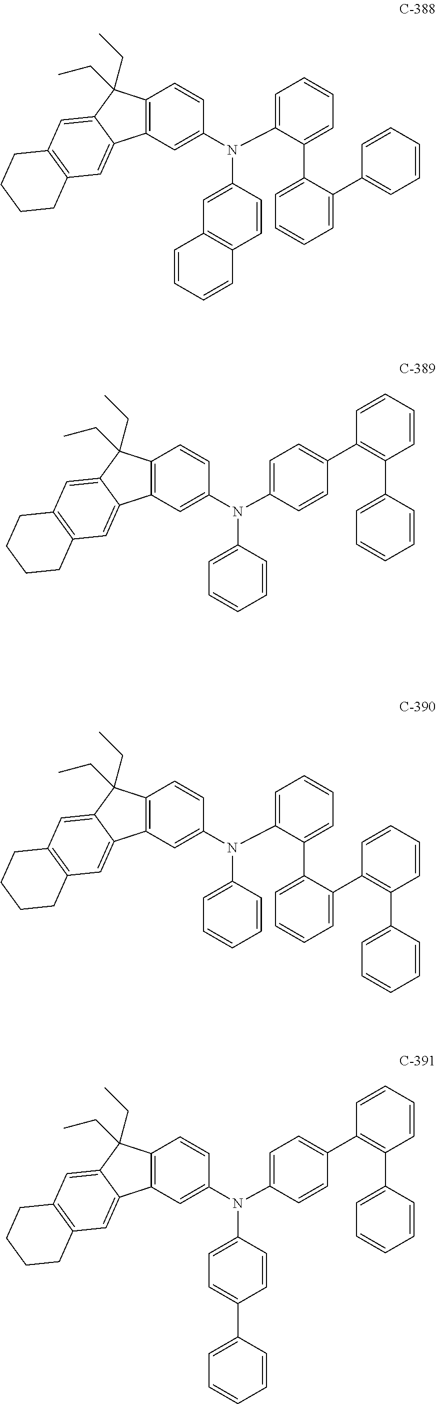 [SMILES: CCC1(CC)c2ccc(N(c3ccc4ccccc4c3)c3ccccc3-c3ccccc3-c3ccccc3)cc2-c2cc3c(cc21)CCCC3 ; CCC1(CC)c2ccc(N(c3ccccc3)c3ccc(-c4ccccc4-c4ccccc4)cc3)cc2-c2cc3c(cc21)CCCC3 ; CCC1(CC)c2ccc(N(c3ccccc3)c3ccccc3-c3ccccc3-c3ccccc3-c3ccccc3)cc2-c2cc3c(cc21)CCCC3 ; CCC1(CC)c2ccc(N(c3ccc(-c4ccccc4)cc3)c3ccc(-c4ccccc4-c4ccccc4)cc3)cc2-c2cc3c(cc21)CCCC3]

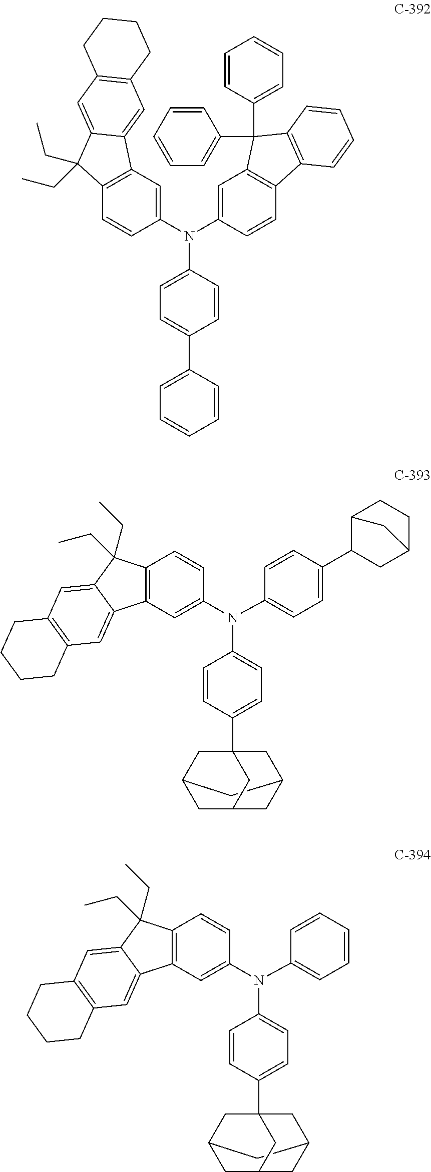 [SMILES: CCC1(CC)c2ccc(N(c3ccc(-c4ccccc4)cc3)c3ccc4c(c3)C(c3ccccc3)(c3ccccc3)c3ccccc3-4)cc2-c2cc3c(cc21)CCCC3 ; CCC1(CC)c2ccc(N(c3ccc(C4CC5CCC4C5)cc3)c3ccc(C45CC6CC(CC(C6)C4)C5)cc3)cc2-c2cc3c(cc21)CCCC3 ; CCC1(CC)c2ccc(N(c3ccccc3)c3ccc(C45CC6CC(CC(C6)C4)C5)cc3)cc2-c2cc3c(cc21)CCCC3]

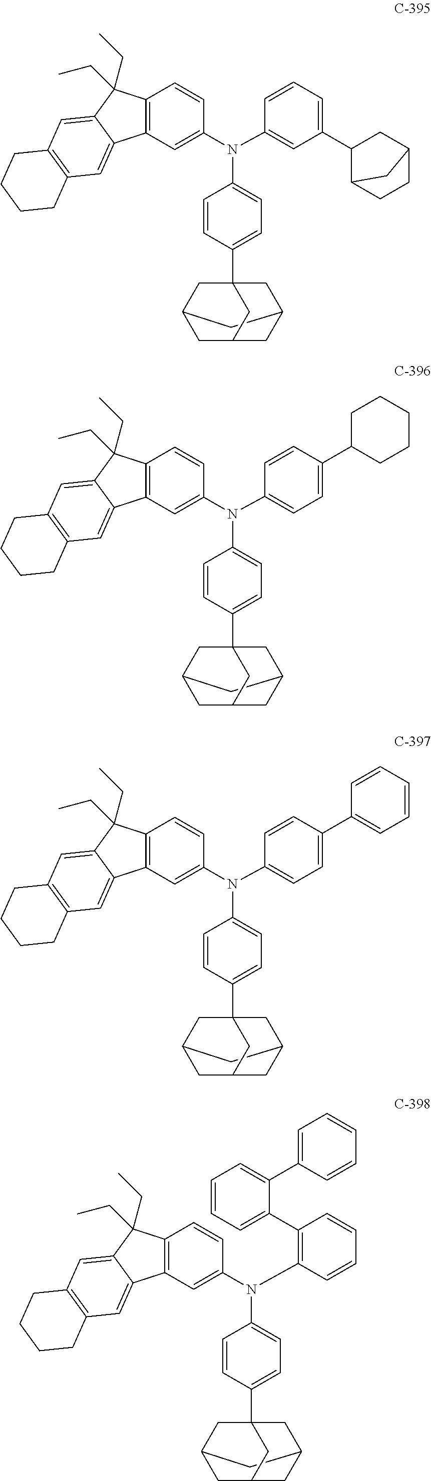 [SMILES: CCC1(CC)c2ccc(N(c3ccc(C45CC6CC(CC(C6)C4)C5)cc3)c3cccc(C4CC5CCC4C5)c3)cc2-c2cc3c(cc21)CCCC3 ; CCC1(CC)c2ccc(N(c3ccc(C4CCCCC4)cc3)c3ccc(C45CC6CC(CC(C6)C4)C5)cc3)cc2-c2cc3c(cc21)CCCC3 ; CCC1(CC)c2ccc(N(c3ccc(-c4ccccc4)cc3)c3ccc(C45CC6CC(CC(C6)C4)C5)cc3)cc2-c2cc3c(cc21)CCCC3 ; CCC1(CC)c2ccc(N(c3ccc(C45CC6CC(CC(C6)C4)C5)cc3)c3ccccc3-c3ccccc3-c3ccccc3)cc2-c2cc3c(cc21)CCCC3]

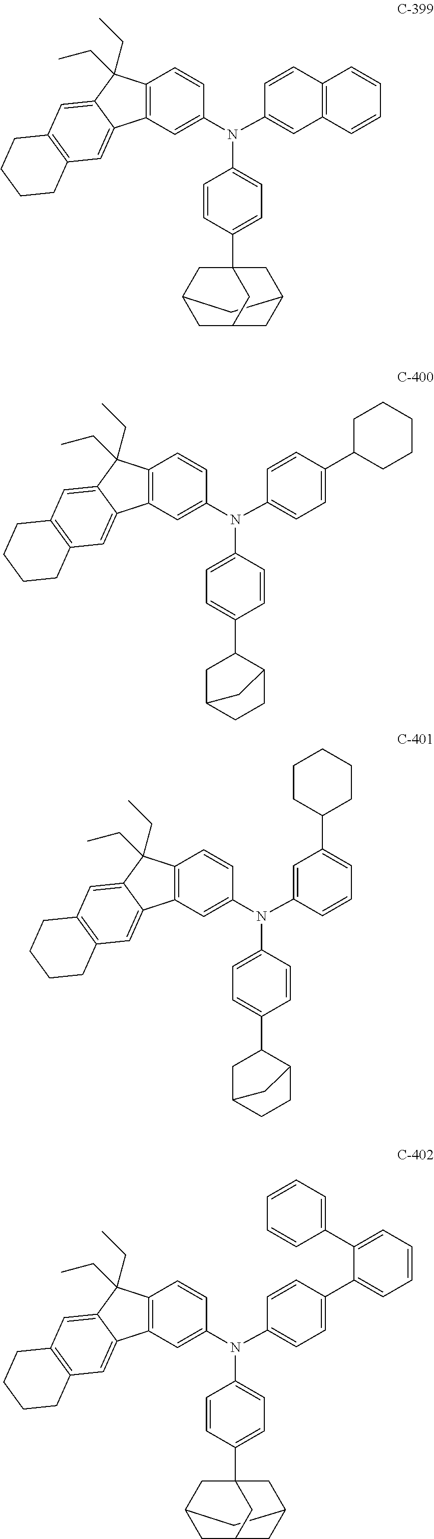 [SMILES: CCC1(CC)c2ccc(N(c3ccc(C45CC6CC(CC(C6)C4)C5)cc3)c3ccc4ccccc4c3)cc2-c2cc3c(cc21)CCCC3 ; CCC1(CC)c2ccc(N(c3ccc(C4CCCCC4)cc3)c3ccc(C4CC5CCC4C5)cc3)cc2-c2cc3c(cc21)CCCC3 ; CCC1(CC)c2ccc(N(c3ccc(C4CC5CCC4C5)cc3)c3cccc(C4CCCCC4)c3)cc2-c2cc3c(cc21)CCCC3 ; CCC1(CC)c2ccc(N(c3ccc(-c4ccccc4-c4ccccc4)cc3)c3ccc(C45CC6CC(CC(C6)C4)C5)cc3)cc2-c2cc3c(cc21)CCCC3]

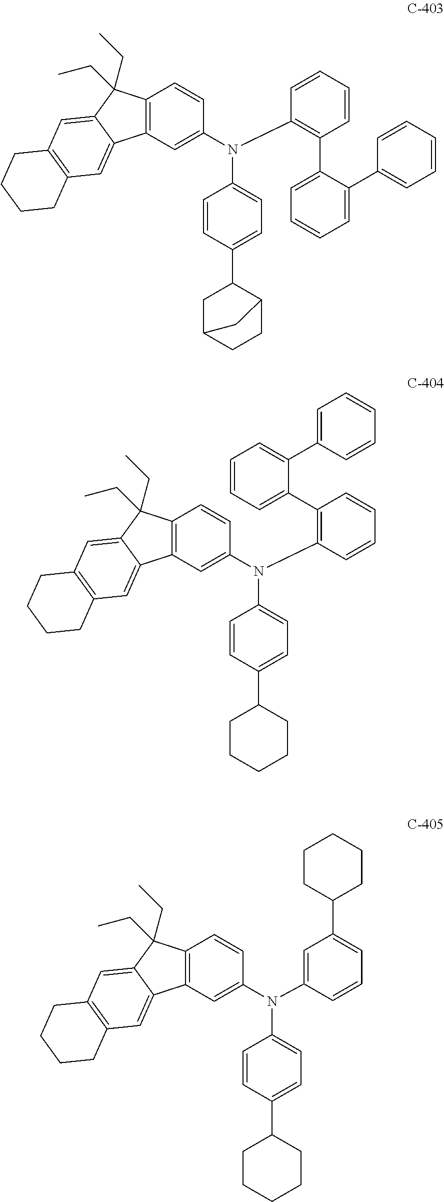 [SMILES: CCC1(CC)c2ccc(N(c3ccc(C4CC5CCC4C5)cc3)c3ccccc3-c3ccccc3-c3ccccc3)cc2-c2cc3c(cc21)CCCC3 ; CCC1(CC)c2ccc(N(c3ccc(C4CCCCC4)cc3)c3ccccc3-c3ccccc3-c3ccccc3)cc2-c2cc3c(cc21)CCCC3 ; CCC1(CC)c2ccc(N(c3ccc(C4CCCCC4)cc3)c3cccc(C4CCCCC4)c3)cc2-c2cc3c(cc21)CCCC3]

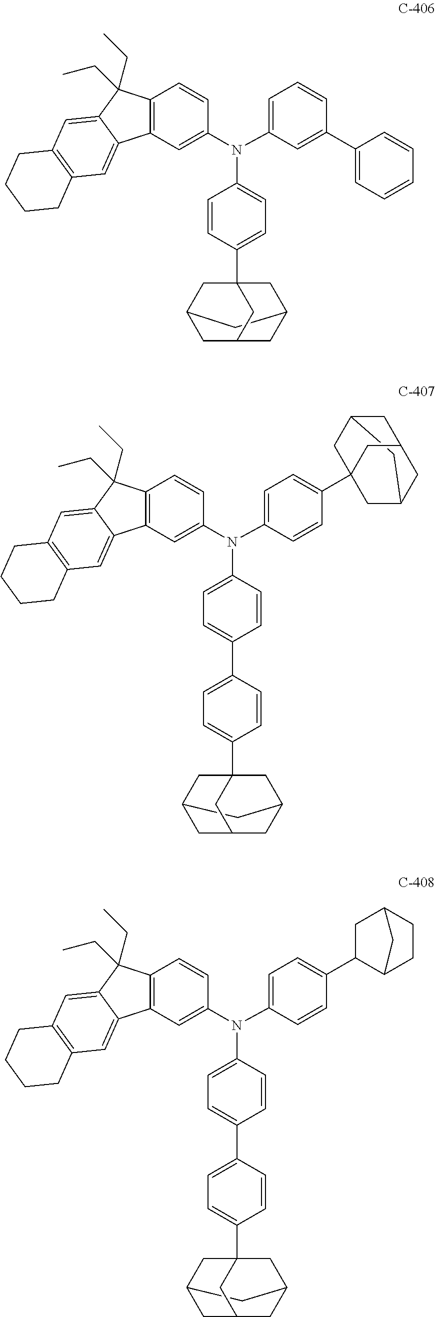 [SMILES: CCC1(CC)c2ccc(N(c3ccc(C45CC6CC(CC(C6)C4)C5)cc3)c3cccc(-c4ccccc4)c3)cc2-c2cc3c(cc21)CCCC3 ; CCC1(CC)c2ccc(N(c3ccc(-c4ccc(C56CC7CC(CC(C7)C5)C6)cc4)cc3)c3ccc(C45CC6CC(CC(C6)C4)C5)cc3)cc2-c2cc3c(cc21)CCCC3 ; CCC1(CC)c2ccc(N(c3ccc(-c4ccc(C56CC7CC(CC(C7)C5)C6)cc4)cc3)c3ccc(C4CC5CCC4C5)cc3)cc2-c2cc3c(cc21)CCCC3]

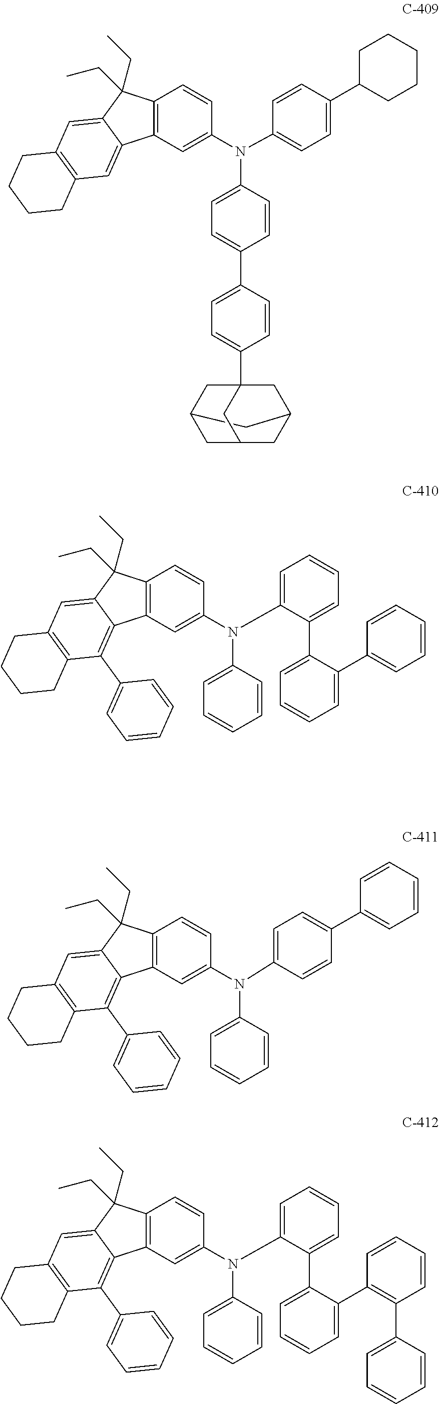 [SMILES: CCC1(CC)c2ccc(N(c3ccc(-c4ccc(C56CC7CC(CC(C7)C5)C6)cc4)cc3)c3ccc(C4CCCCC4)cc3)cc2-c2cc3c(cc21)CCCC3 ; CCC1(CC)c2ccc(N(c3ccccc3)c3ccccc3-c3ccccc3-c3ccccc3)cc2-c2c1cc1c(c2-c2ccccc2)CCCC1 ; CCC1(CC)c2ccc(N(c3ccccc3)c3ccc(-c4ccccc4)cc3)cc2-c2c1cc1c(c2-c2ccccc2)CCCC1 ; CCC1(CC)c2ccc(N(c3ccccc3)c3ccccc3-c3ccccc3-c3ccccc3-c3ccccc3)cc2-c2c1cc1c(c2-c2ccccc2)CCCC1]

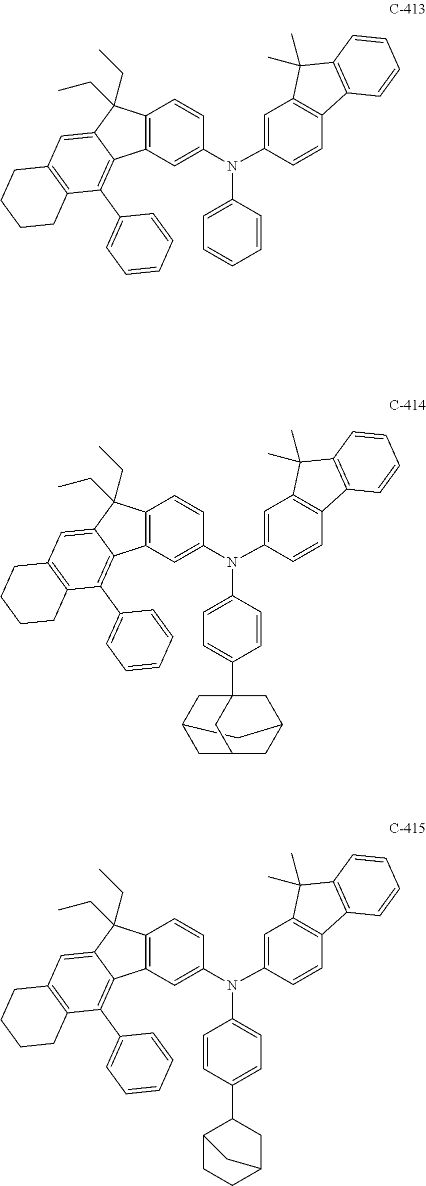 [SMILES: CCC1(CC)c2ccc(N(c3ccccc3)c3ccc4c(c3)C(C)(C)c3ccccc3-4)cc2-c2c1cc1c(c2-c2ccccc2)CCCC1 ; CCC1(CC)c2ccc(N(c3ccc(C45CC6CC(CC(C6)C4)C5)cc3)c3ccc4c(c3)C(C)(C)c3ccccc3-4)cc2-c2c1cc1c(c2-c2ccccc2)CCCC1 ; CCC1(CC)c2ccc(N(c3ccc(C4CC5CCC4C5)cc3)c3ccc4c(c3)C(C)(C)c3ccccc3-4)cc2-c2c1cc1c(c2-c2ccccc2)CCCC1]

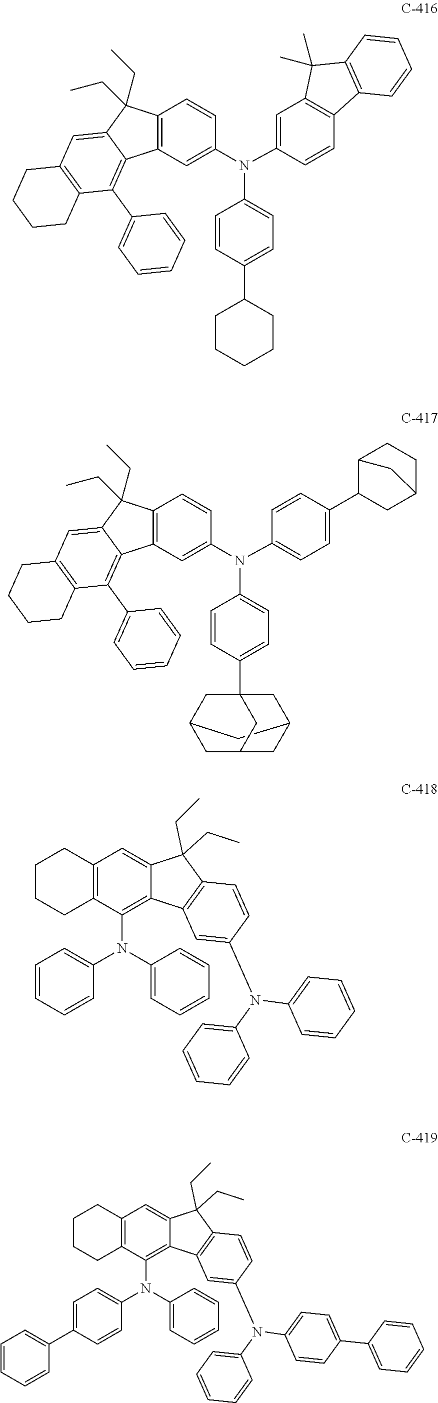 [SMILES: CCC1(CC)c2ccc(N(c3ccc(C4CCCCC4)cc3)c3ccc4c(c3)C(C)(C)c3ccccc3-4)cc2-c2c1cc1c(c2-c2ccccc2)CCCC1 ; CCC1(CC)c2ccc(N(c3ccc(C4CC5CCC4C5)cc3)c3ccc(C45CC6CC(CC(C6)C4)C5)cc3)cc2-c2c1cc1c(c2-c2ccccc2)CCCC1 ; CCC1(CC)c2ccc(N(c3ccccc3)c3ccccc3)cc2-c2c1cc1c(c2N(c2ccccc2)c2ccccc2)CCCC1 ; CCC1(CC)c2ccc(N(c3ccccc3)c3ccc(-c4ccccc4)cc3)cc2-c2c1cc1c(c2N(c2ccccc2)c2ccc(-c3ccccc3)cc2)CCCC1]

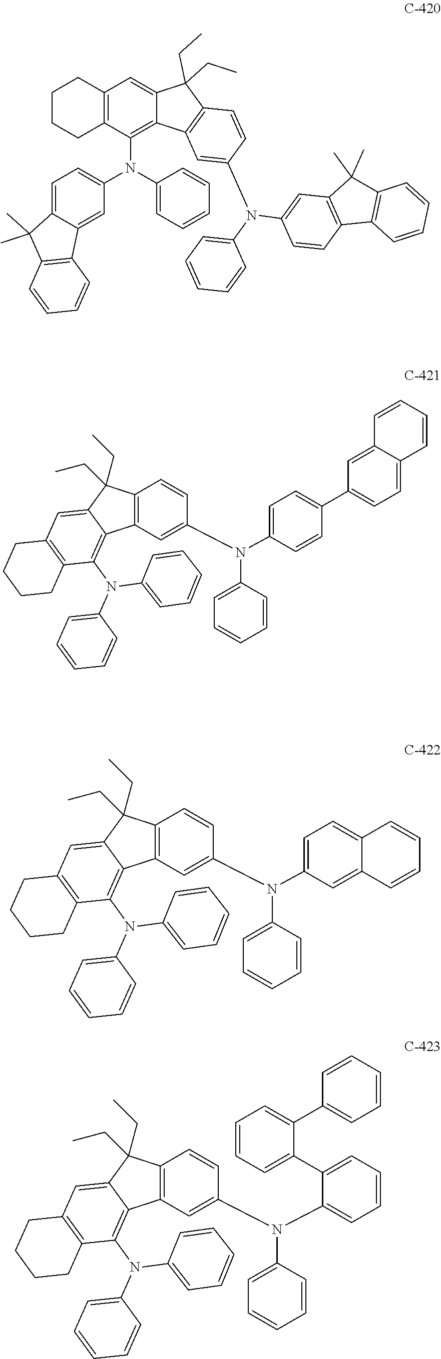 [SMILES: CCC1(CC)c2ccc(N(c3ccccc3)c3ccc4c(c3)C(C)(C)c3ccccc3-4)cc2-c2c1cc1c(c2N(c2ccccc2)c2ccc3c(c2)-c2ccccc2C3(C)C)CCCC1 ; CCC1(CC)c2ccc(N(c3ccccc3)c3ccc(-c4ccc5ccccc5c4)cc3)cc2-c2c1cc1c(c2N(c2ccccc2)c2ccccc2)CCCC1 ; CCC1(CC)c2ccc(N(c3ccccc3)c3ccc4ccccc4c3)cc2-c2c1cc1c(c2N(c2ccccc2)c2ccccc2)CCCC1 ; CCC1(CC)c2ccc(N(c3ccccc3)c3ccccc3-c3ccccc3-c3ccccc3)cc2-c2c1cc1c(c2N(c2ccccc2)c2ccccc2)CCCC1]

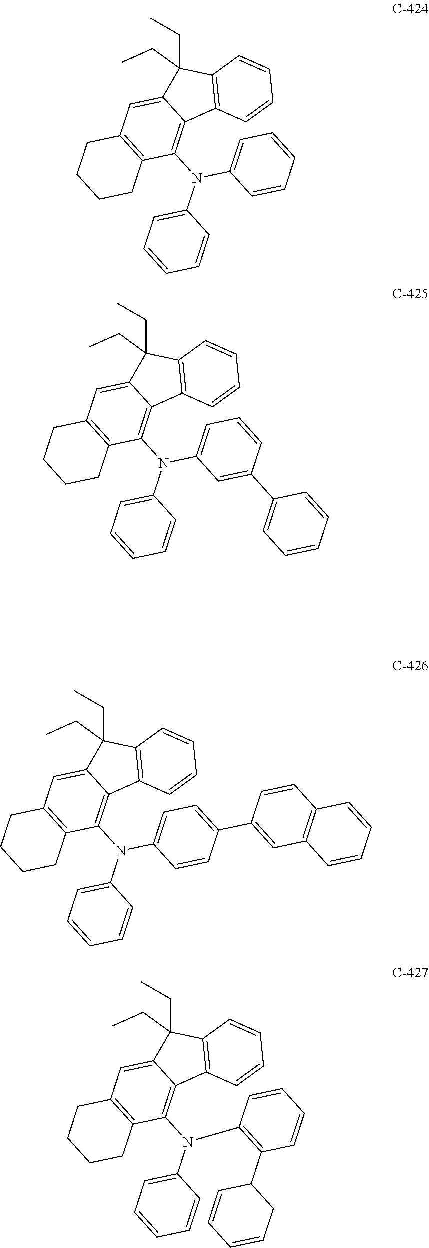 [SMILES: CCC1(CC)c2ccccc2-c2c1cc1c(c2N(c2ccccc2)c2ccccc2)CCCC1 ; CCC1(CC)c2ccccc2-c2c1cc1c(c2N(c2ccccc2)c2cccc(-c3ccccc3)c2)CCCC1 ; CCC1(CC)c2ccccc2-c2c1cc1c(c2N(c2ccccc2)c2ccc(-c3ccc4ccccc4c3)cc2)CCCC1 ; CCC1(CC)c2ccccc2-c2c1cc1c(c2N(c2ccccc2)c2ccccc2C2C=CC=CC2)CCCC1]

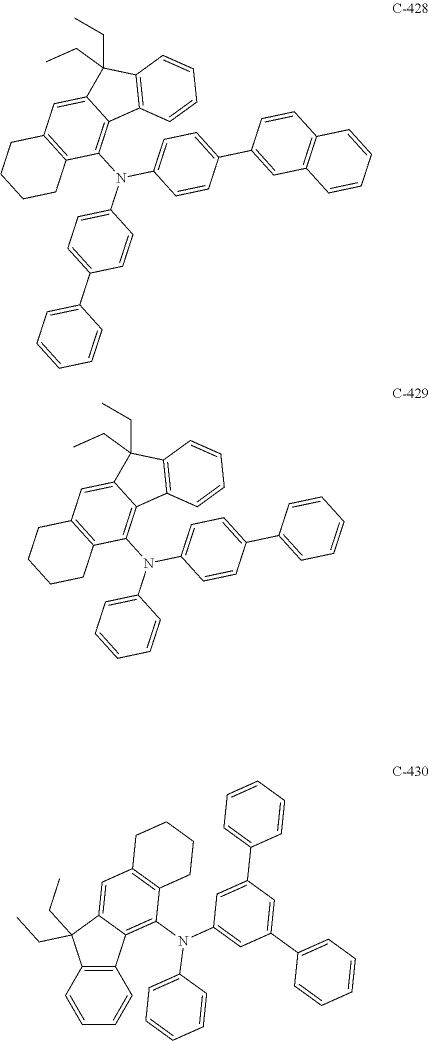

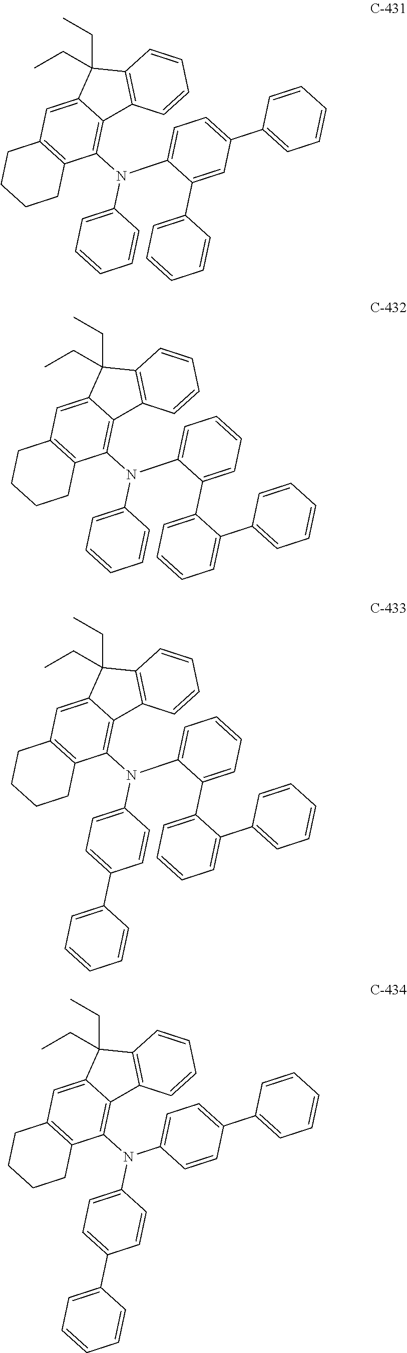

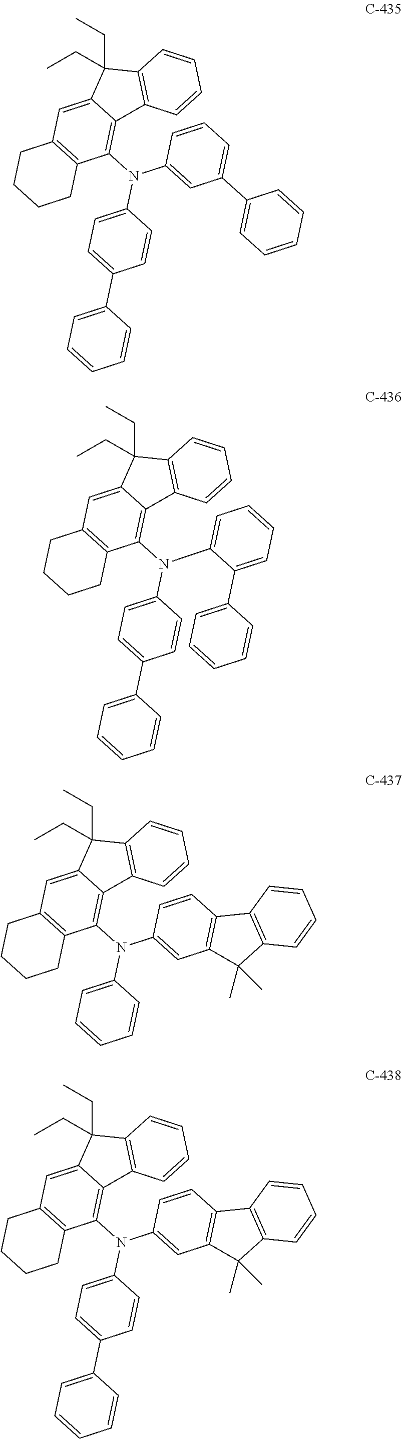

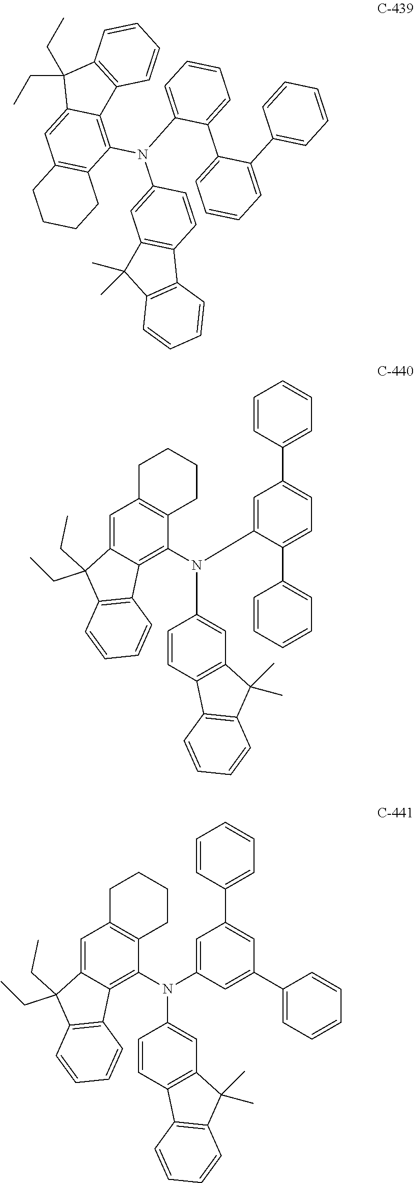 [SMILES: CCC1(CC)c2ccccc2-c2c1cc1c(c2N(c2ccc3c(c2)C(C)(C)c2ccccc2-3)c2ccccc2-c2ccccc2-c2ccccc2)CCCC1 ; CCC1(CC)c2ccccc2-c2c1cc1c(c2N(c2ccc3c(c2)C(C)(C)c2ccccc2-3)c2cc(-c3ccccc3)ccc2-c2ccccc2)CCCC1 ; CCC1(CC)c2ccccc2-c2c1cc1c(c2N(c2cc(-c3ccccc3)cc(-c3ccccc3)c2)c2ccc3c(c2)C(C)(C)c2ccccc2-3)CCCC1]

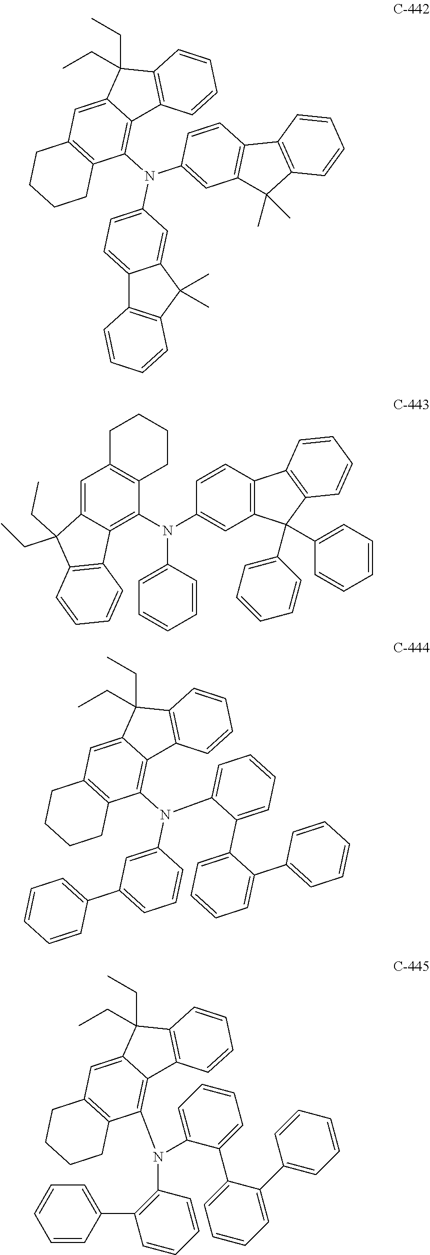

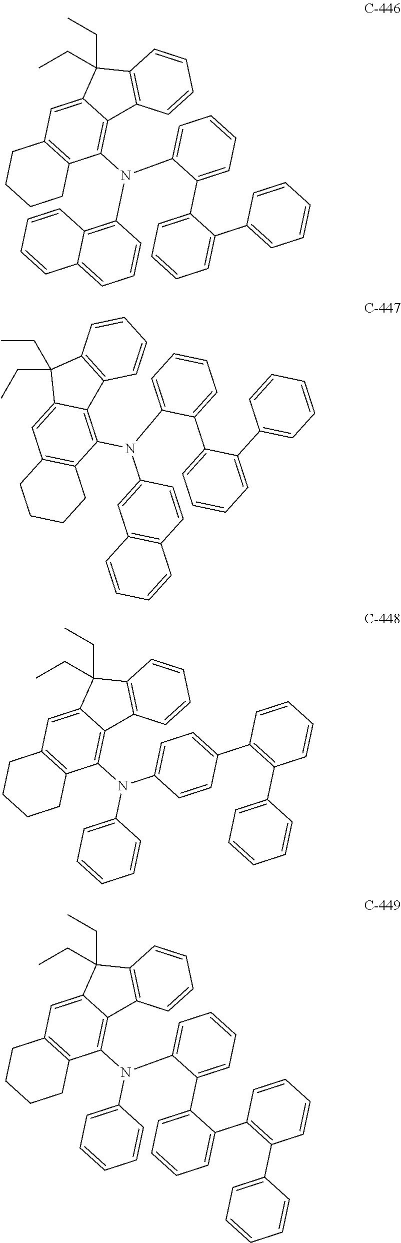

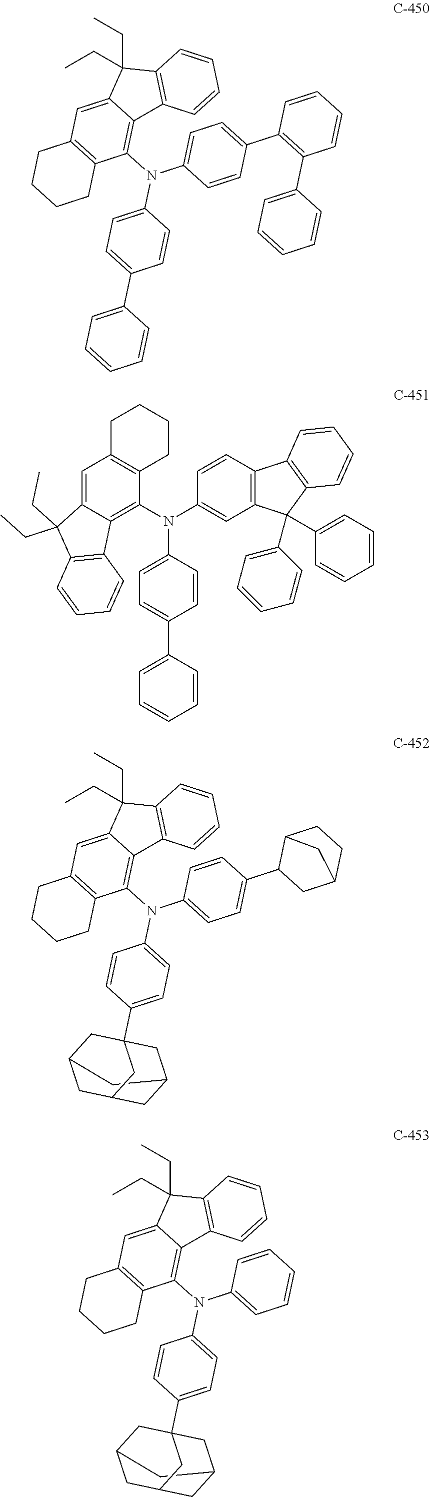

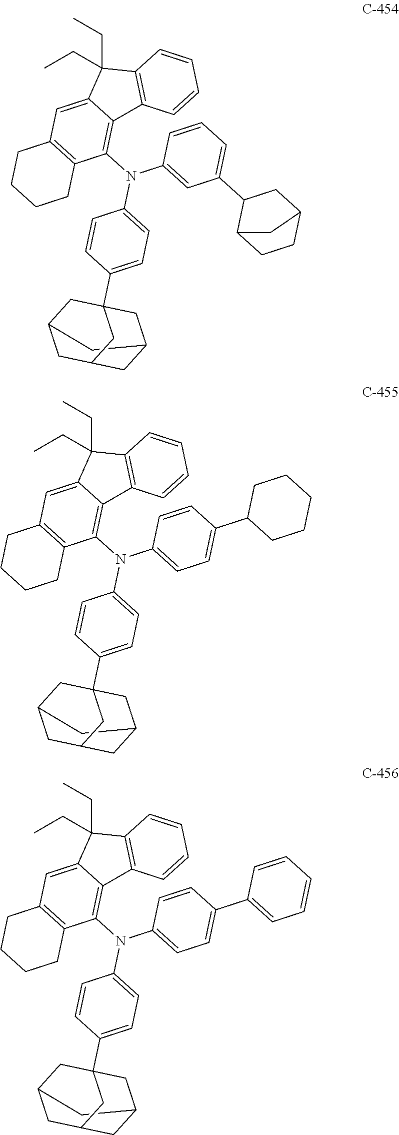

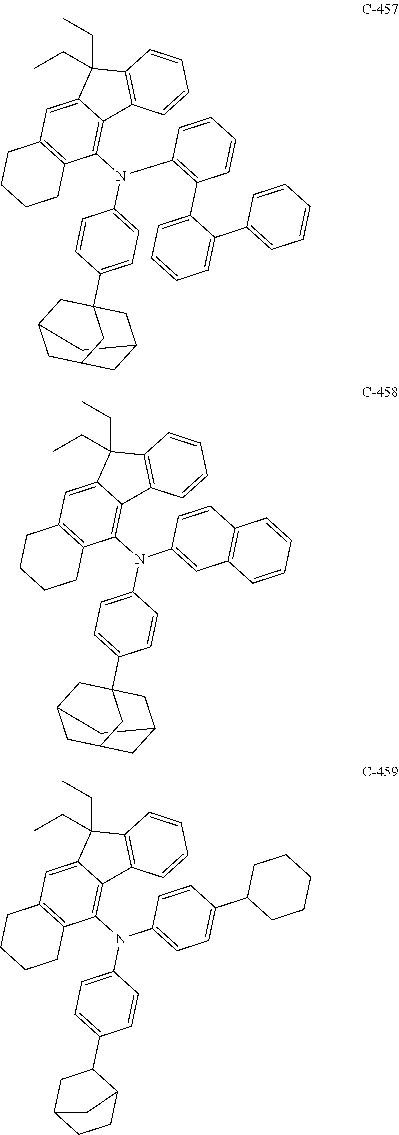

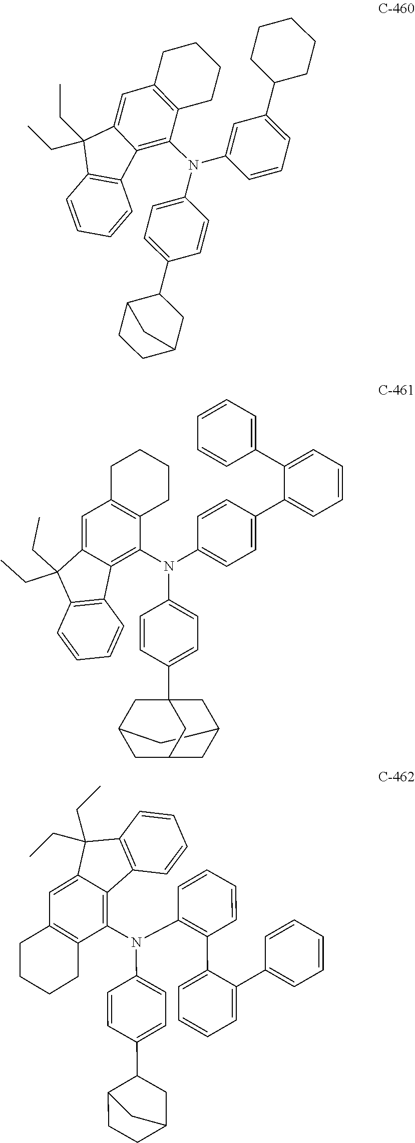

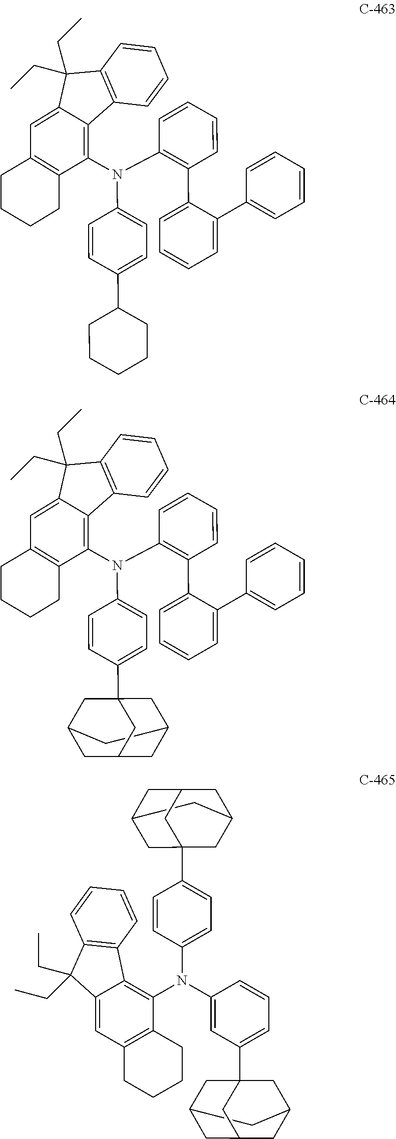

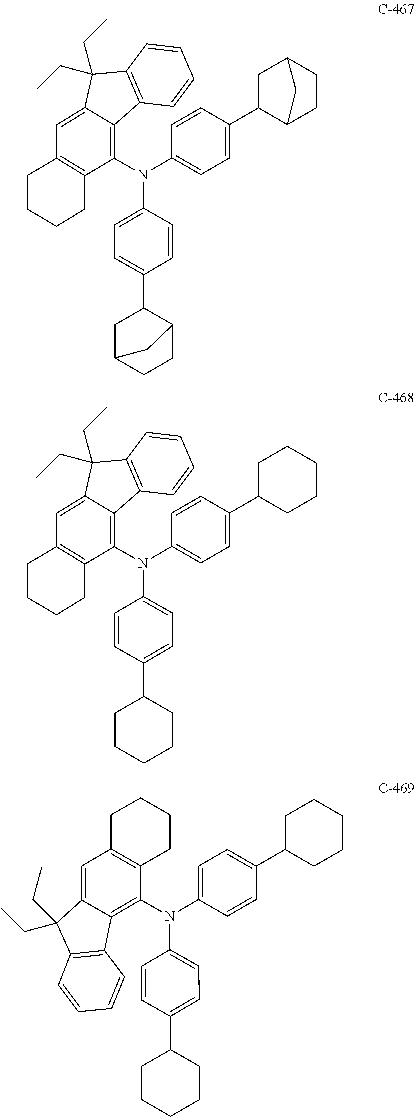

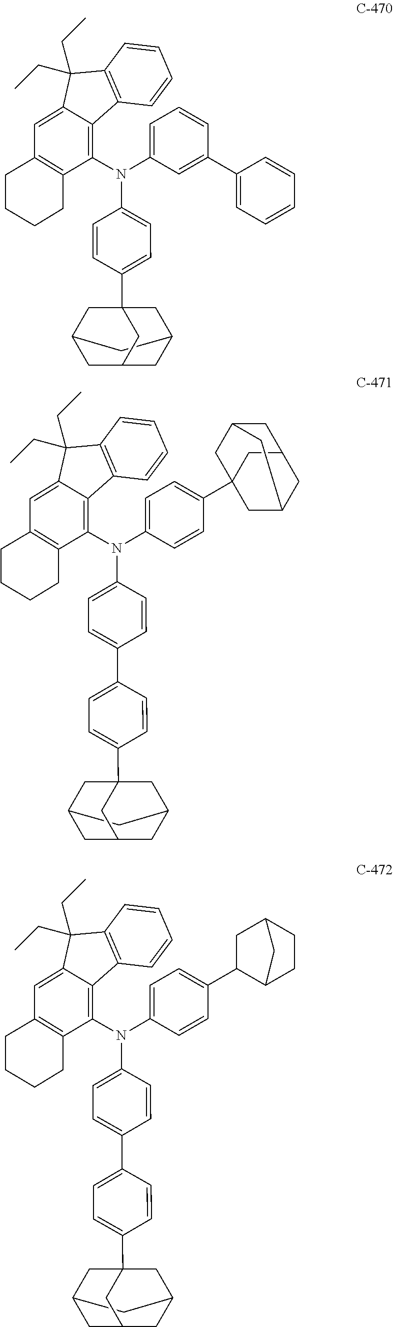

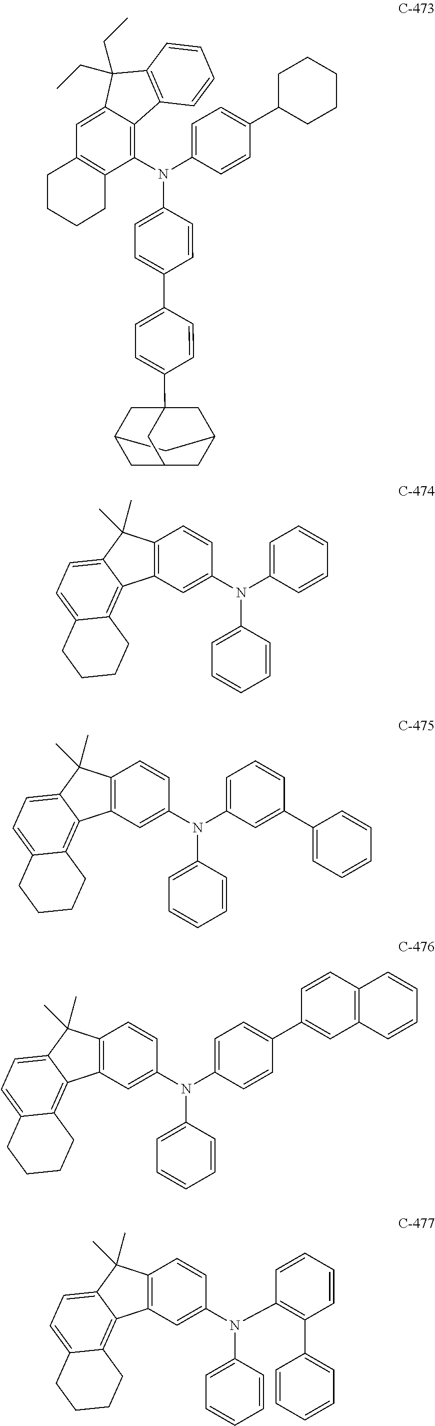

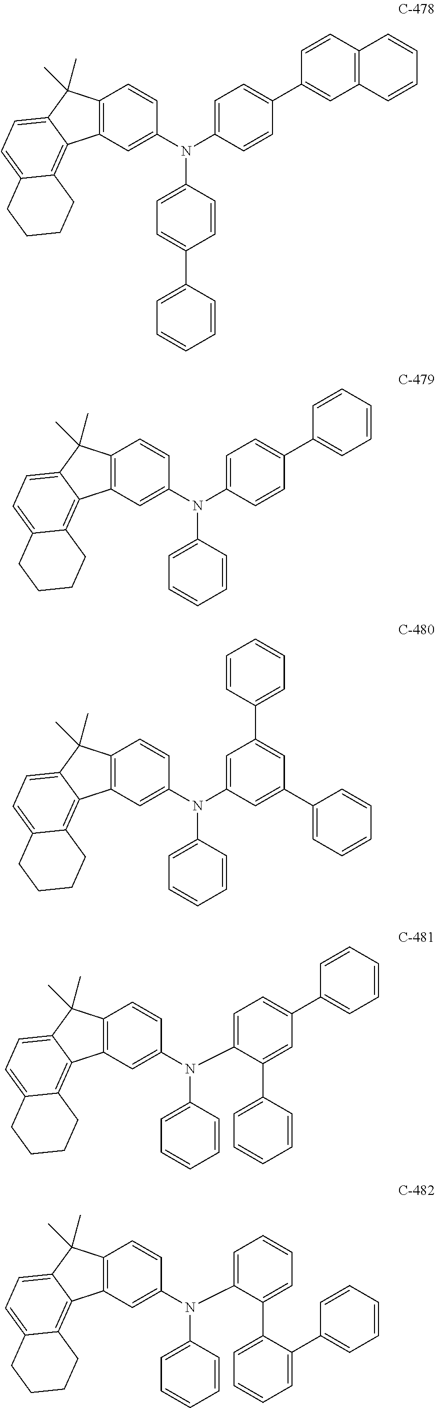 [SMILES: CC1(C)c2ccc(N(c3ccc(-c4ccccc4)cc3)c3ccc(-c4ccc5ccccc5c4)cc3)cc2-c2c1ccc1c2CCCC1 ; CC1(C)c2ccc(N(c3ccccc3)c3ccc(-c4ccccc4)cc3)cc2-c2c1ccc1c2CCCC1 ; CC1(C)c2ccc(N(c3ccccc3)c3cc(-c4ccccc4)cc(-c4ccccc4)c3)cc2-c2c1ccc1c2CCCC1 ; CC1(C)c2ccc(N(c3ccccc3)c3ccc(-c4ccccc4)cc3-c3ccccc3)cc2-c2c1ccc1c2CCCC1 ; CC1(C)c2ccc(N(c3ccccc3)c3ccccc3-c3ccccc3-c3ccccc3)cc2-c2c1ccc1c2CCCC1]

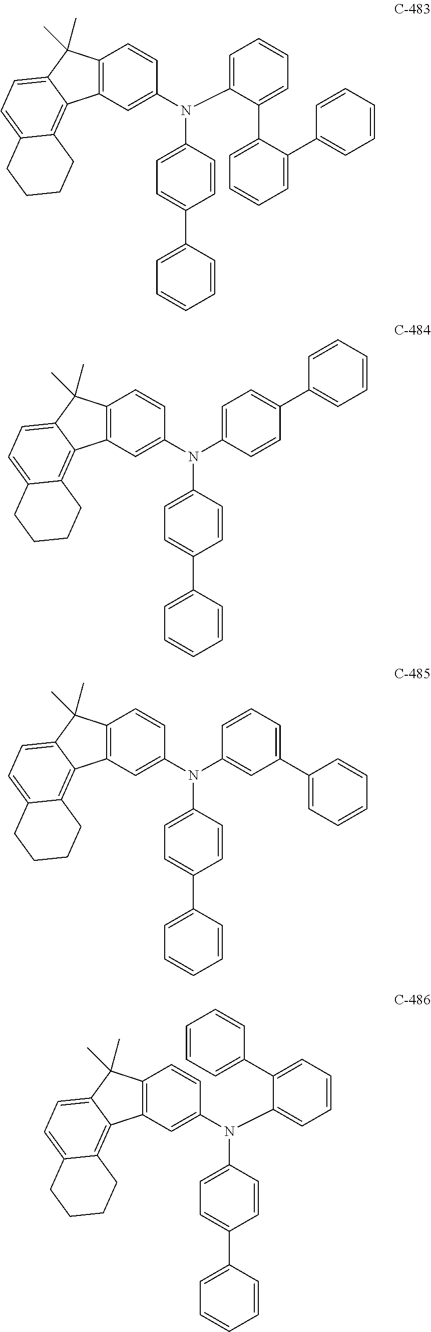

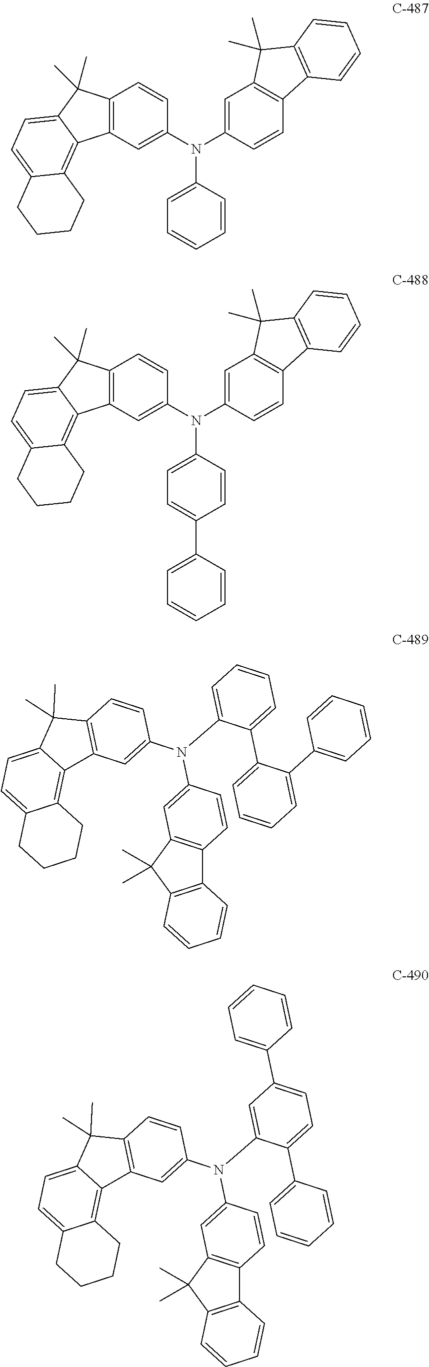 [SMILES: CC1(C)c2ccccc2-c2ccc(N(c3ccccc3)c3ccc4c(c3)-c3c(ccc5c3CCCC5)C4(C)C)cc21 ; CC1(C)c2ccccc2-c2ccc(N(c3ccc(-c4ccccc4)cc3)c3ccc4c(c3)-c3c(ccc5c3CCCC5)C4(C)C)cc21 ; CC1(C)c2ccccc2-c2ccc(N(c3ccc4c(c3)-c3c(ccc5c3CCCC5)C4(C)C)c3ccccc3-c3ccccc3-c3ccccc3)cc21 ; CC1(C)c2ccccc2-c2ccc(N(c3ccc4c(c3)-c3c(ccc5c3CCCC5)C4(C)C)c3cc(-c4ccccc4)ccc3-c3ccccc3)cc21]

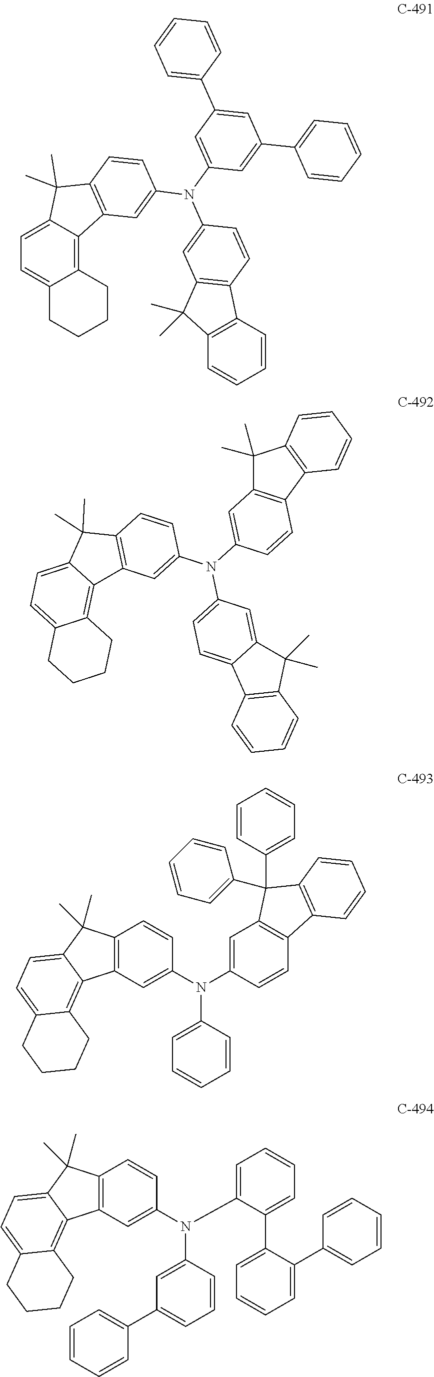

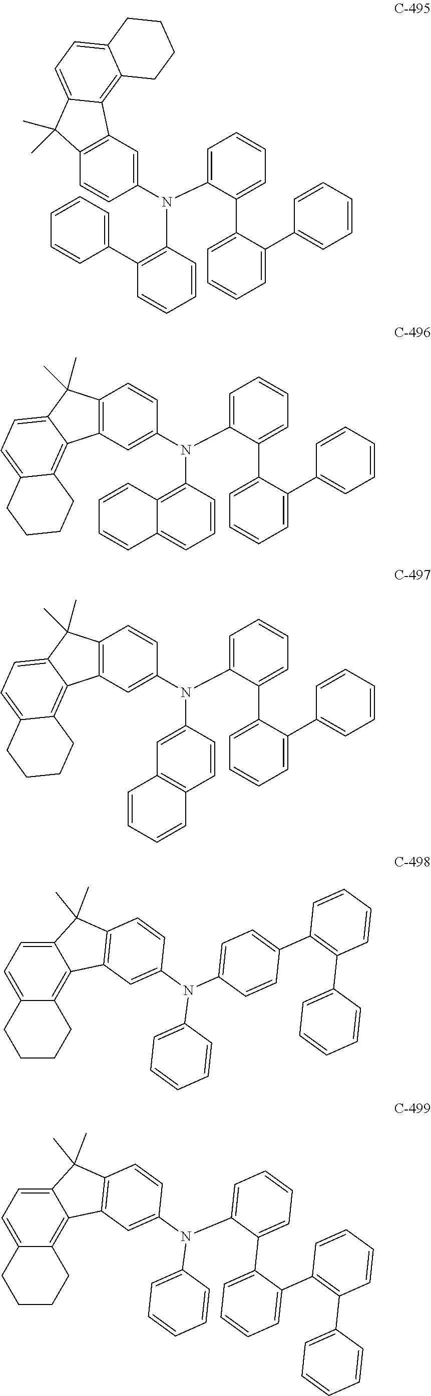 [SMILES: CC1(C)c2ccc(N(c3ccccc3-c3ccccc3)c3ccccc3-c3ccccc3-c3ccccc3)cc2-c2c1ccc1c2CCCC1 ; CC1(C)c2ccc(N(c3ccccc3-c3ccccc3-c3ccccc3)c3cccc4ccccc34)cc2-c2c1ccc1c2CCCC1 ; CC1(C)c2ccc(N(c3ccc4ccccc4c3)c3ccccc3-c3ccccc3-c3ccccc3)cc2-c2c1ccc1c2CCCC1 ; CC1(C)c2ccc(N(c3ccccc3)c3ccc(-c4ccccc4-c4ccccc4)cc3)cc2-c2c1ccc1c2CCCC1 ; CC1(C)c2ccc(N(c3ccccc3)c3ccccc3-c3ccccc3-c3ccccc3-c3ccccc3)cc2-c2c1ccc1c2CCCC1]

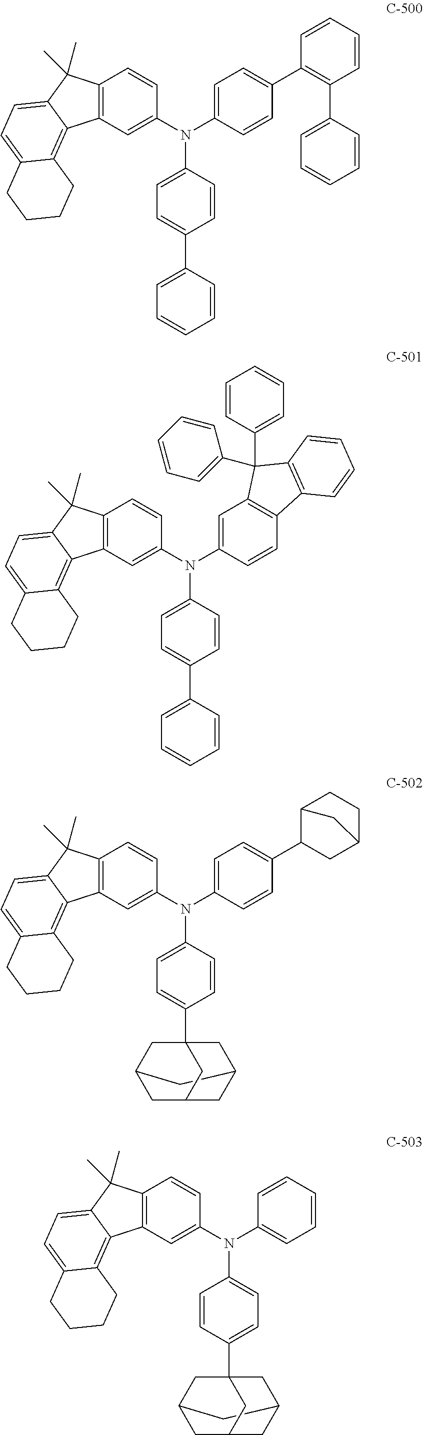 [SMILES: CC1(C)c2ccc(N(c3ccc(-c4ccccc4)cc3)c3ccc(-c4ccccc4-c4ccccc4)cc3)cc2-c2c1ccc1c2CCCC1 ; CC1(C)c2ccc(N(c3ccc(-c4ccccc4)cc3)c3ccc4c(c3)C(c3ccccc3)(c3ccccc3)c3ccccc3-4)cc2-c2c1ccc1c2CCCC1 ; CC1(C)c2ccc(N(c3ccc(C4CC5CCC4C5)cc3)c3ccc(C45CC6CC(CC(C6)C4)C5)cc3)cc2-c2c1ccc1c2CCCC1 ; CC1(C)c2ccc(N(c3ccccc3)c3ccc(C45CC6CC(CC(C6)C4)C5)cc3)cc2-c2c1ccc1c2CCCC1]

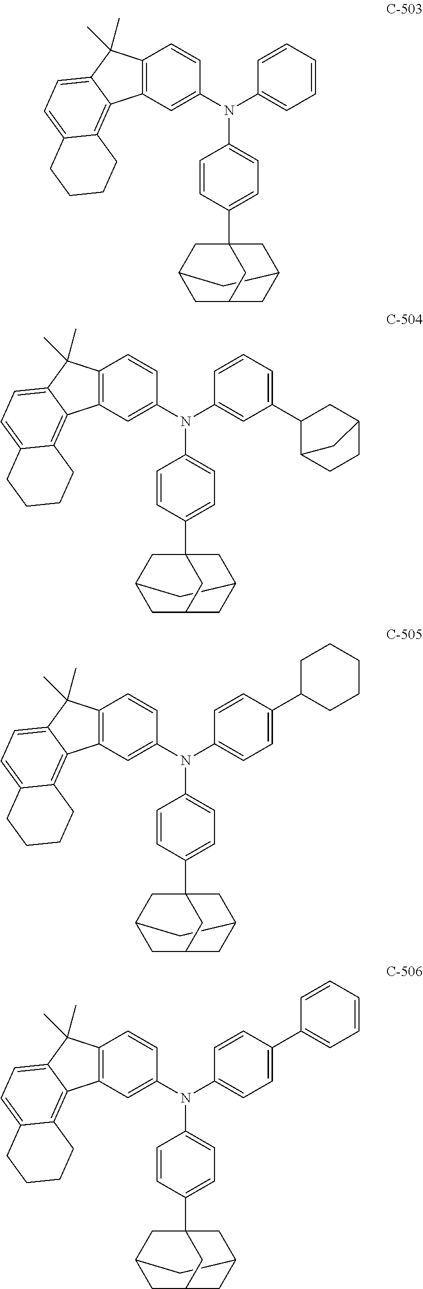 [SMILES: CC1(C)c2ccc(N(c3ccccc3)c3ccc(C45CC6CC(CC(C6)C4)C5)cc3)cc2-c2c1ccc1c2CCCC1 ; CC1(C)c2ccc(N(c3ccc(C45CC6CC(CC(C6)C4)C5)cc3)c3cccc(C4CC5CCC4C5)c3)cc2-c2c1ccc1c2CCCC1 ; CC1(C)c2ccc(N(c3ccc(C4CCCCC4)cc3)c3ccc(C45CC6CC(CC(C6)C4)C5)cc3)cc2-c2c1ccc1c2CCCC1 ; CC1(C)c2ccc(N(c3ccc(-c4ccccc4)cc3)c3ccc(C45CC6CC(CC(C6)C4)C5)cc3)cc2-c2c1ccc1c2CCCC1]

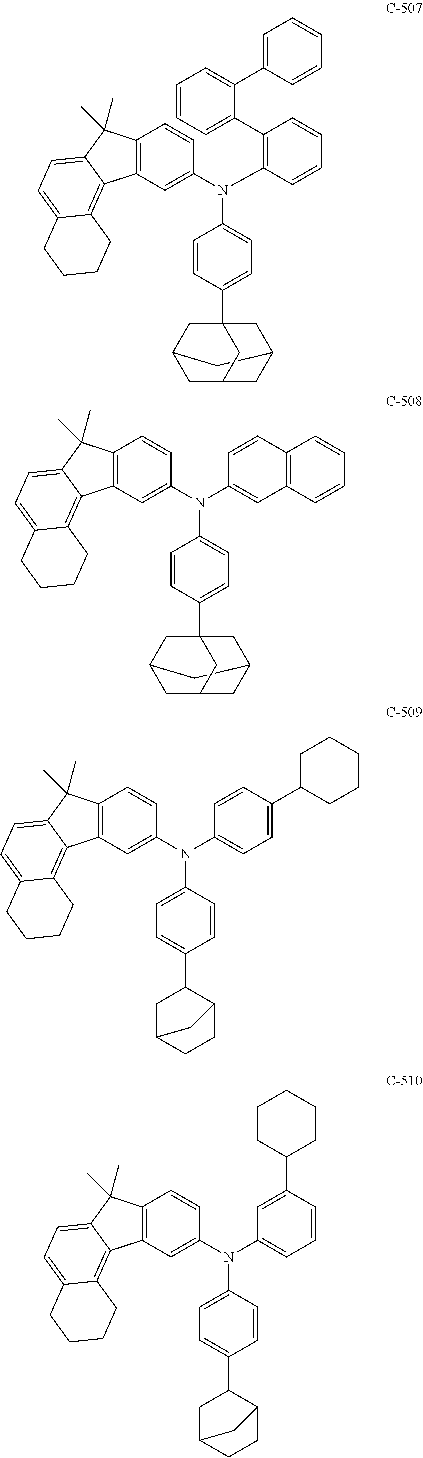 [SMILES: CC1(C)c2ccc(N(c3ccc(C45CC6CC(CC(C6)C4)C5)cc3)c3ccccc3-c3ccccc3-c3ccccc3)cc2-c2c1ccc1c2CCCC1 ; CC1(C)c2ccc(N(c3ccc(C45CC6CC(CC(C6)C4)C5)cc3)c3ccc4ccccc4c3)cc2-c2c1ccc1c2CCCC1 ; CC1(C)c2ccc(N(c3ccc(C4CCCCC4)cc3)c3ccc(C4CC5CCC4C5)cc3)cc2-c2c1ccc1c2CCCC1 ; CC1(C)c2ccc(N(c3ccc(C4CC5CCC4C5)cc3)c3cccc(C4CCCCC4)c3)cc2-c2c1ccc1c2CCCC1]

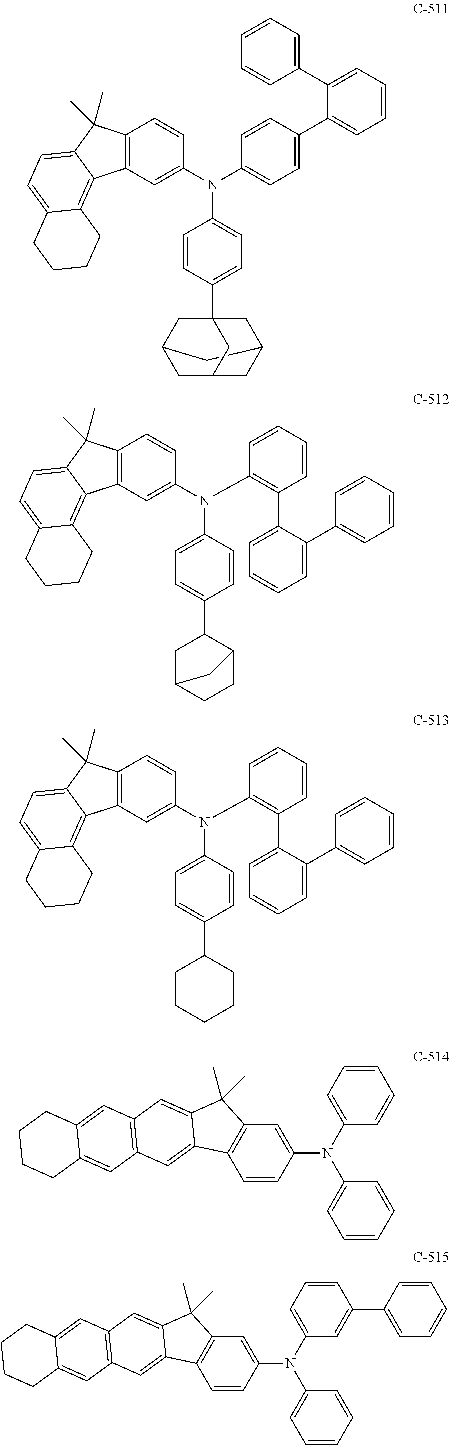

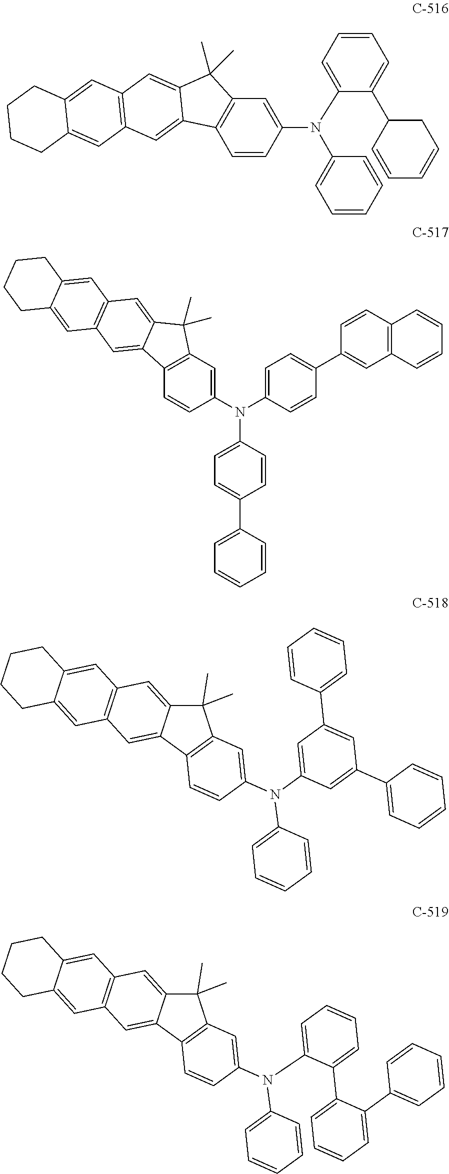 [SMILES: CC1(C)c2cc(N(c3ccccc3)c3ccccc3C3C=CC=CC3)ccc2-c2cc3cc4c(cc3cc21)CCCC4 ; CC1(C)c2cc(N(c3ccc(-c4ccccc4)cc3)c3ccc(-c4ccc5ccccc5c4)cc3)ccc2-c2cc3cc4c(cc3cc21)CCCC4 ; CC1(C)c2cc(N(c3ccccc3)c3cc(-c4ccccc4)cc(-c4ccccc4)c3)ccc2-c2cc3cc4c(cc3cc21)CCCC4 ; CC1(C)c2cc(N(c3ccccc3)c3ccccc3-c3ccccc3-c3ccccc3)ccc2-c2cc3cc4c(cc3cc21)CCCC4]

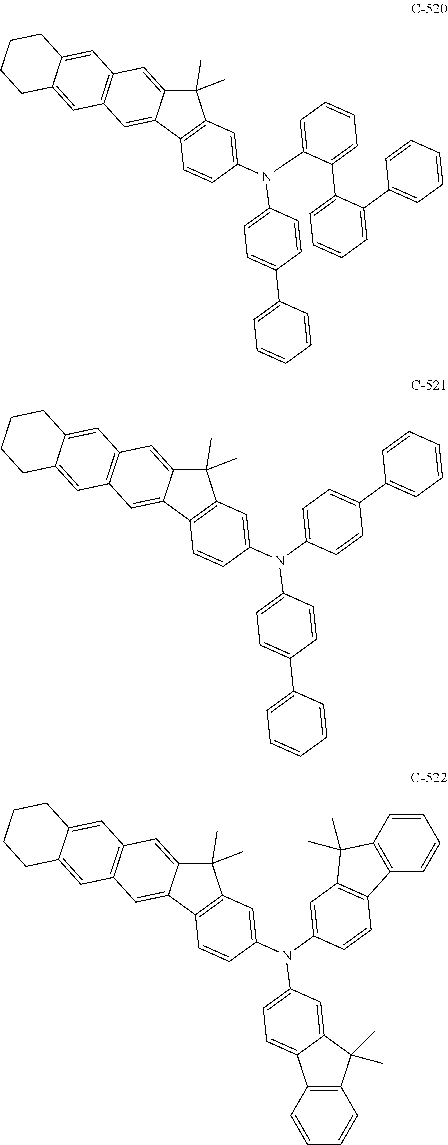 [SMILES: CC1(C)c2cc(N(c3ccc(-c4ccccc4)cc3)c3ccccc3-c3ccccc3-c3ccccc3)ccc2-c2cc3cc4c(cc3cc21)CCCC4 ; CC1(C)c2cc(N(c3ccc(-c4ccccc4)cc3)c3ccc(-c4ccccc4)cc3)ccc2-c2cc3cc4c(cc3cc21)CCCC4 ; CC1(C)c2ccccc2-c2ccc(N(c3ccc4c(c3)C(C)(C)c3ccccc3-4)c3ccc4c(c3)C(C)(C)c3cc5cc6c(cc5cc3-4)CCCC6)cc21]

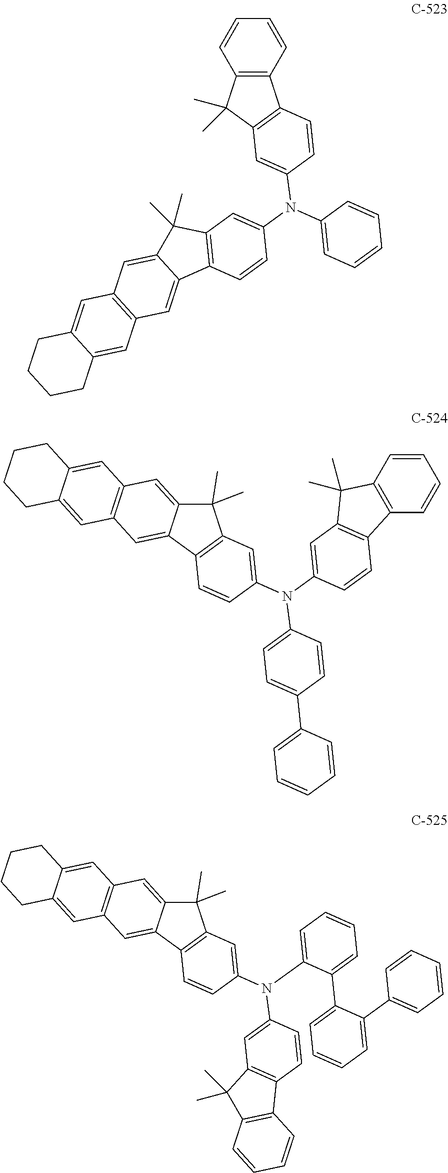 [SMILES: CC1(C)c2ccccc2-c2ccc(N(c3ccccc3)c3ccc4c(c3)C(C)(C)c3cc5cc6c(cc5cc3-4)CCCC6)cc21 ; CC1(C)c2ccccc2-c2ccc(N(c3ccc(-c4ccccc4)cc3)c3ccc4c(c3)C(C)(C)c3cc5cc6c(cc5cc3-4)CCCC6)cc21 ; CC1(C)c2ccccc2-c2ccc(N(c3ccc4c(c3)C(C)(C)c3cc5cc6c(cc5cc3-4)CCCC6)c3ccccc3-c3ccccc3-c3ccccc3)cc21]

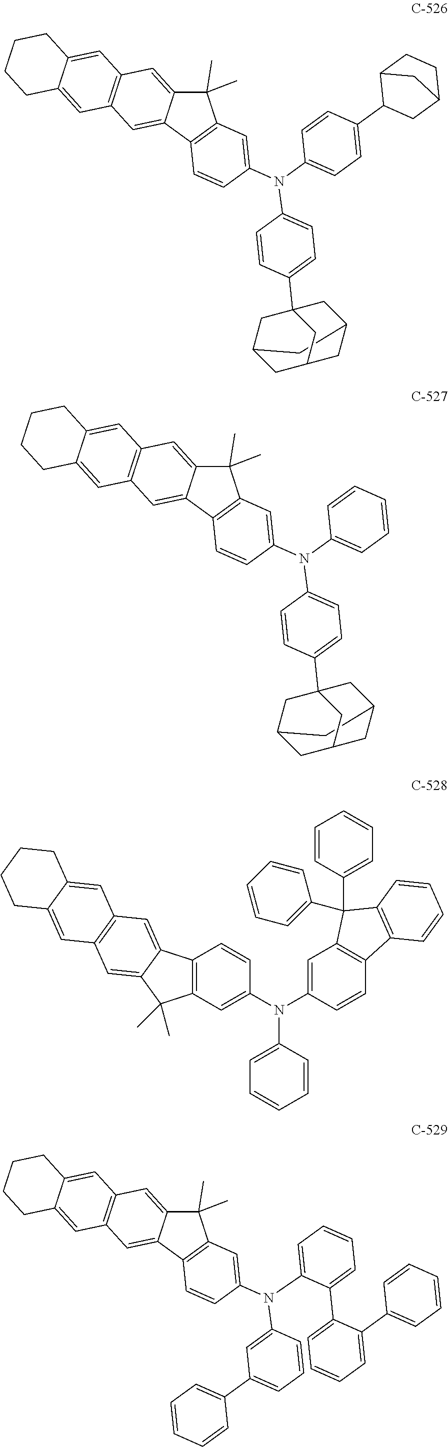 [SMILES: CC1(C)c2cc(N(c3ccc(C4CC5CCC4C5)cc3)c3ccc(C45CC6CC(CC(C6)C4)C5)cc3)ccc2-c2cc3cc4c(cc3cc21)CCCC4 ; CC1(C)c2cc(N(c3ccccc3)c3ccc(C45CC6CC(CC(C6)C4)C5)cc3)ccc2-c2cc3cc4c(cc3cc21)CCCC4 ; CC1(C)c2cc(N(c3ccccc3)c3ccc4c(c3)C(c3ccccc3)(c3ccccc3)c3ccccc3-4)ccc2-c2cc3cc4c(cc3cc21)CCCC4 ; CC1(C)c2cc(N(c3cccc(-c4ccccc4)c3)c3ccccc3-c3ccccc3-c3ccccc3)ccc2-c2cc3cc4c(cc3cc21)CCCC4]

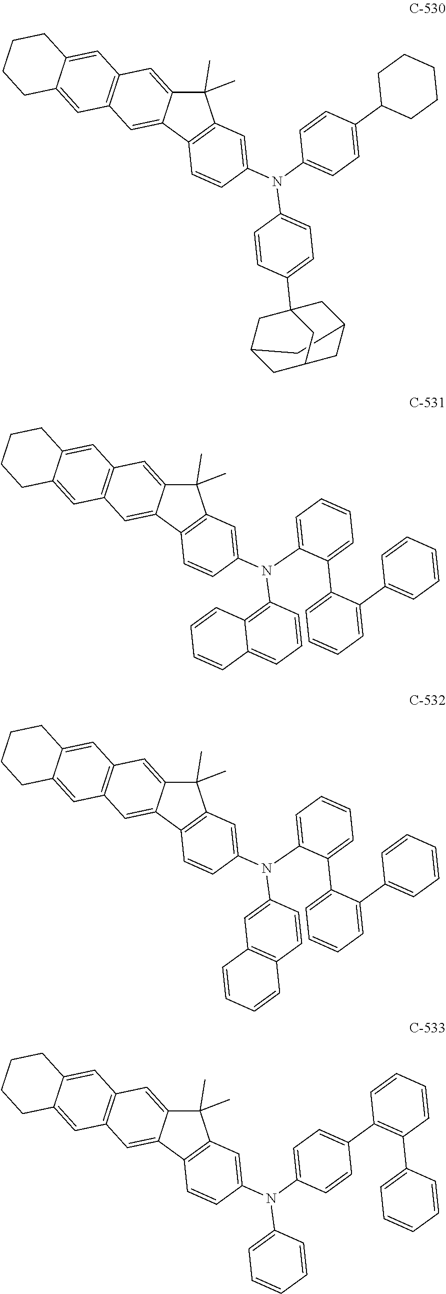 [SMILES: CC1(C)c2cc(N(c3ccc(C4CCCCC4)cc3)c3ccc(C45CC6CC(CC(C6)C4)C5)cc3)ccc2-c2cc3cc4c(cc3cc21)CCCC4 ; CC1(C)c2cc(N(c3ccccc3-c3ccccc3-c3ccccc3)c3cccc4ccccc34)ccc2-c2cc3cc4c(cc3cc21)CCCC4 ; CC1(C)c2cc(N(c3ccc4ccccc4c3)c3ccccc3-c3ccccc3-c3ccccc3)ccc2-c2cc3cc4c(cc3cc21)CCCC4 ; CC1(C)c2cc(N(c3ccccc3)c3ccc(-c4ccccc4-c4ccccc4)cc3)ccc2-c2cc3cc4c(cc3cc21)CCCC4]

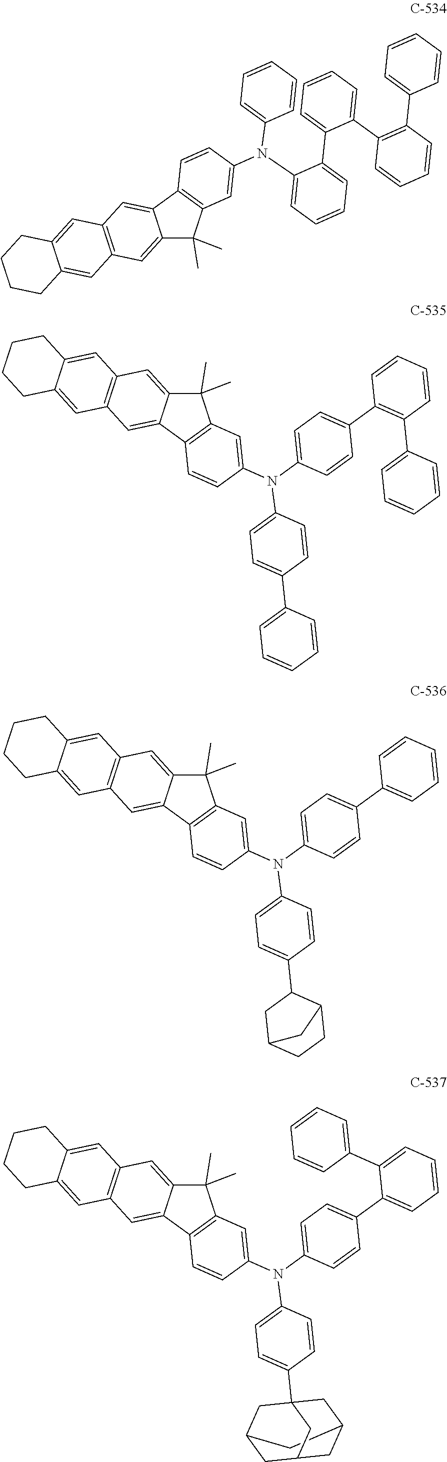

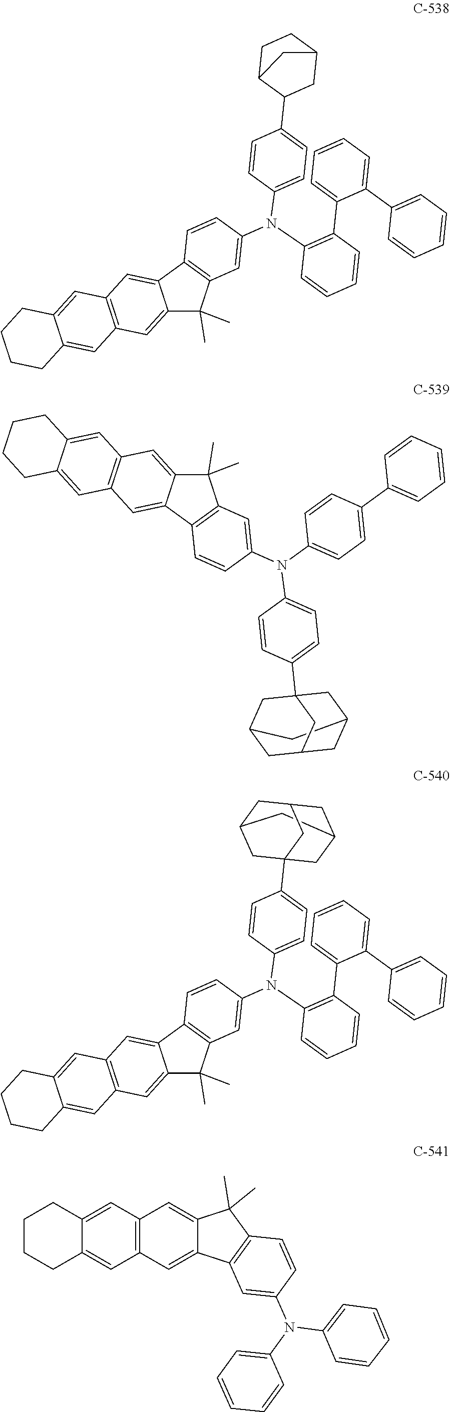 [SMILES: CC1(C)c2cc(N(c3ccc(C4CC5CCC4C5)cc3)c3ccccc3-c3ccccc3-c3ccccc3)ccc2-c2cc3cc4c(cc3cc21)CCCC4 ; CC1(C)c2cc(N(c3ccc(-c4ccccc4)cc3)c3ccc(C45CC6CC(CC(C6)C4)C5)cc3)ccc2-c2cc3cc4c(cc3cc21)CCCC4 ; CC1(C)c2cc(N(c3ccc(C45CC6CC(CC(C6)C4)C5)cc3)c3ccccc3-c3ccccc3-c3ccccc3)ccc2-c2cc3cc4c(cc3cc21)CCCC4 ; CC1(C)c2ccc(N(c3ccccc3)c3ccccc3)cc2-c2cc3cc4c(cc3cc21)CCCC4]

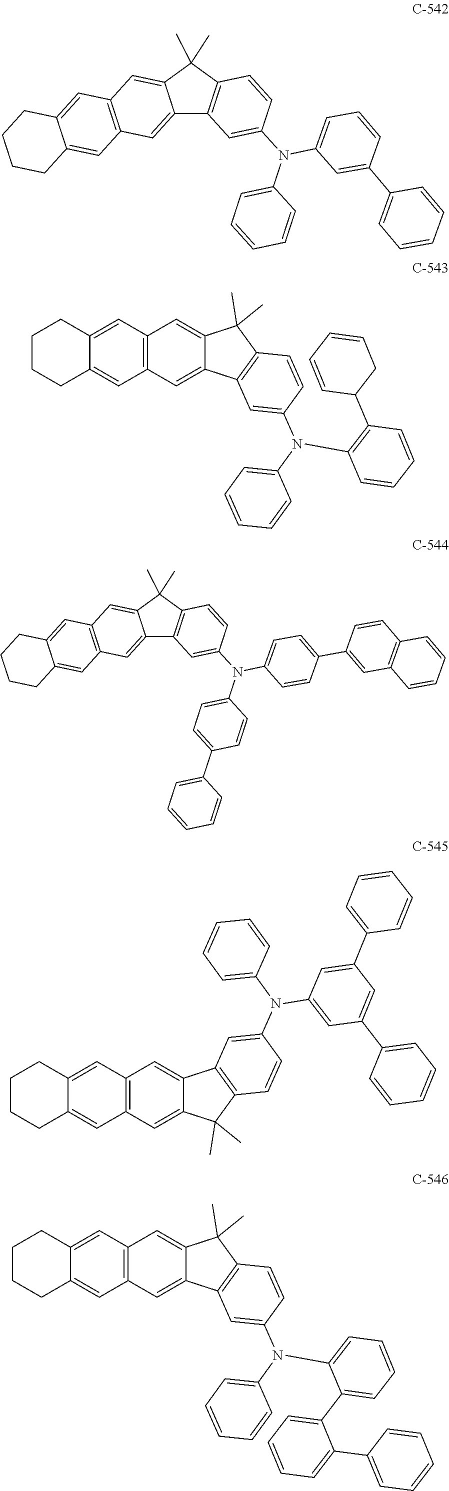 [SMILES: CC1(C)c2ccc(N(c3ccccc3)c3cccc(-c4ccccc4)c3)cc2-c2cc3cc4c(cc3cc21)CCCC4 ; CC1(C)c2ccc(N(c3ccccc3)c3ccccc3C3C=CC=CC3)cc2-c2cc3cc4c(cc3cc21)CCCC4 ; CC1(C)c2ccc(N(c3ccc(-c4ccccc4)cc3)c3ccc(-c4ccc5ccccc5c4)cc3)cc2-c2cc3cc4c(cc3cc21)CCCC4 ; CC1(C)c2ccc(N(c3ccccc3)c3cc(-c4ccccc4)cc(-c4ccccc4)c3)cc2-c2cc3cc4c(cc3cc21)CCCC4 ; CC1(C)c2ccc(N(c3ccccc3)c3ccccc3-c3ccccc3-c3ccccc3)cc2-c2cc3cc4c(cc3cc21)CCCC4]

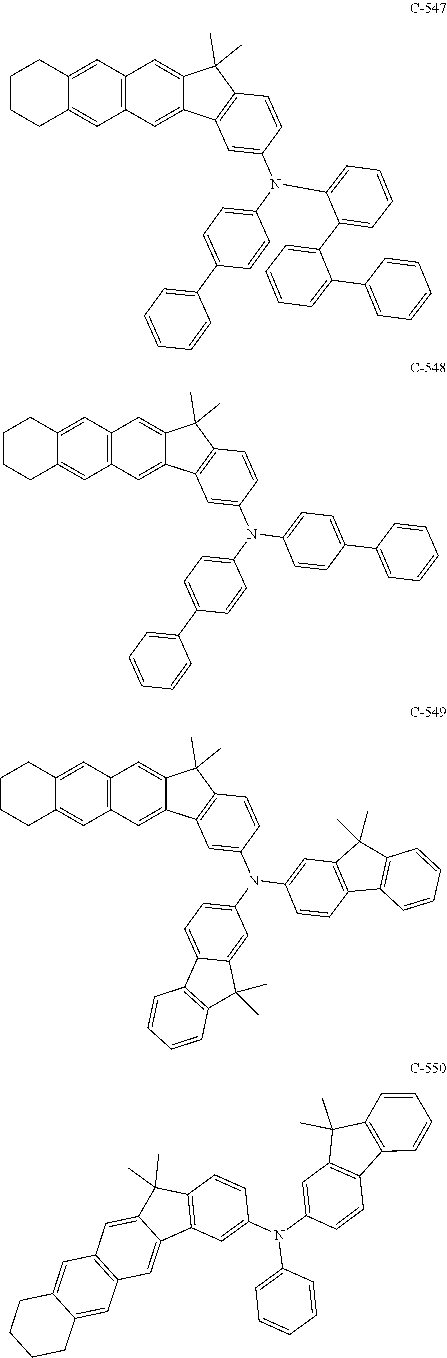

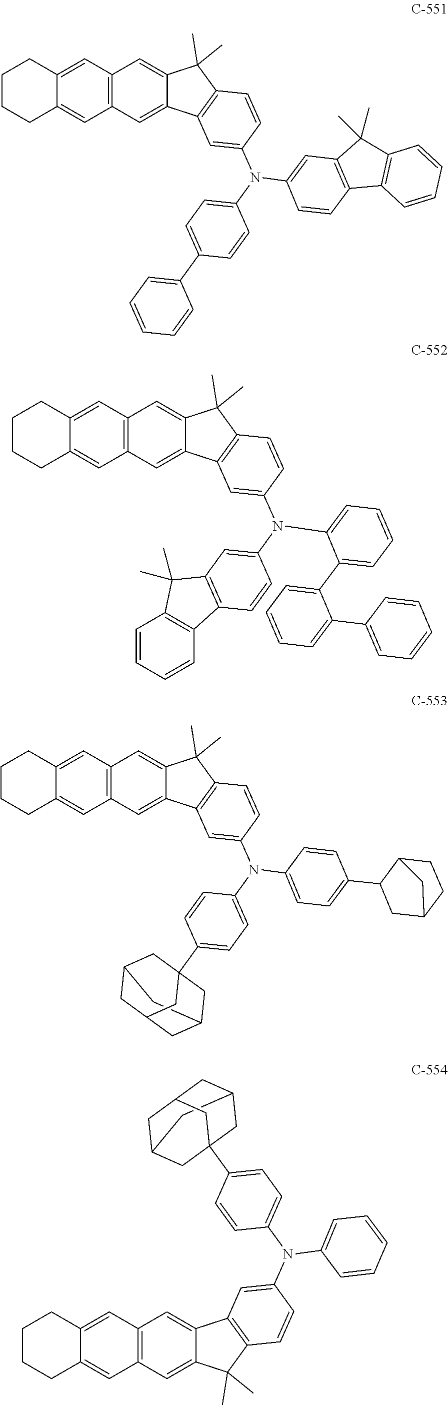 [SMILES: CC1(C)c2ccccc2-c2ccc(N(c3ccc(-c4ccccc4)cc3)c3ccc4c(c3)-c3cc5cc6c(cc5cc3C4(C)C)CCCC6)cc21 ; CC1(C)c2ccccc2-c2ccc(N(c3ccc4c(c3)-c3cc5cc6c(cc5cc3C4(C)C)CCCC6)c3ccccc3-c3ccccc3-c3ccccc3)cc21 ; CC1(C)c2ccc(N(c3ccc(C4CC5CCC4C5)cc3)c3ccc(C45CC6CC(CC(C6)C4)C5)cc3)cc2-c2cc3cc4c(cc3cc21)CCCC4 ; CC1(C)c2ccc(N(c3ccccc3)c3ccc(C45CC6CC(CC(C6)C4)C5)cc3)cc2-c2cc3cc4c(cc3cc21)CCCC4]

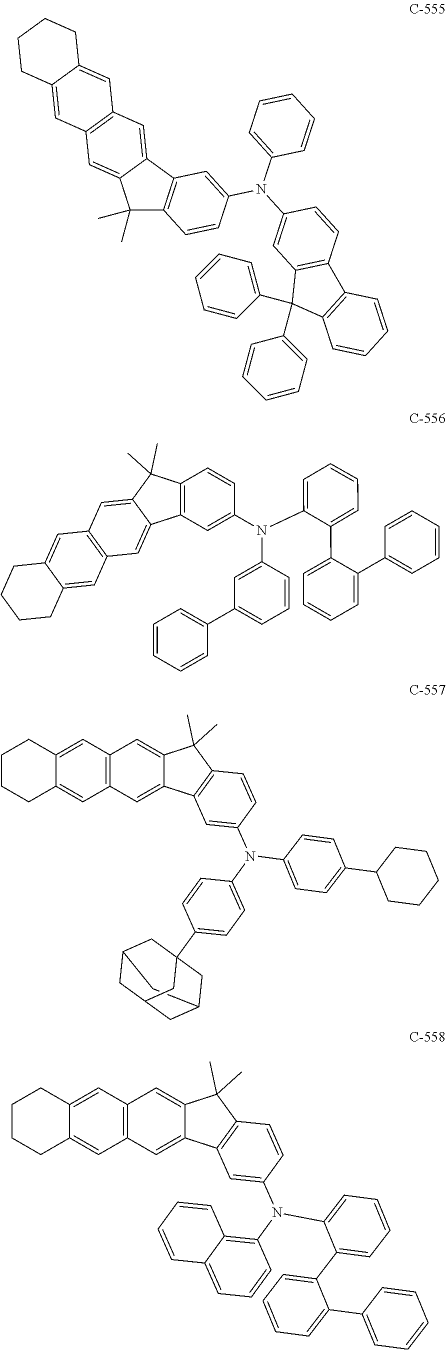

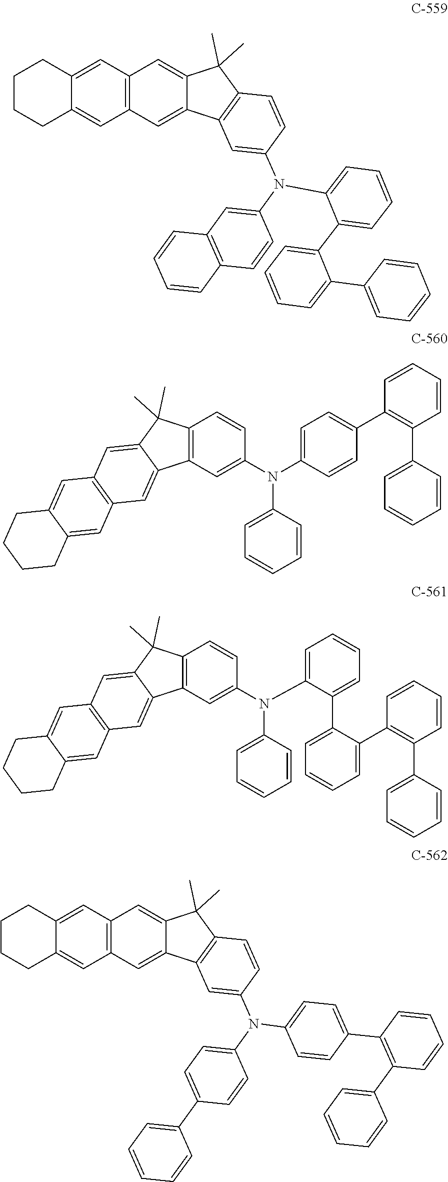

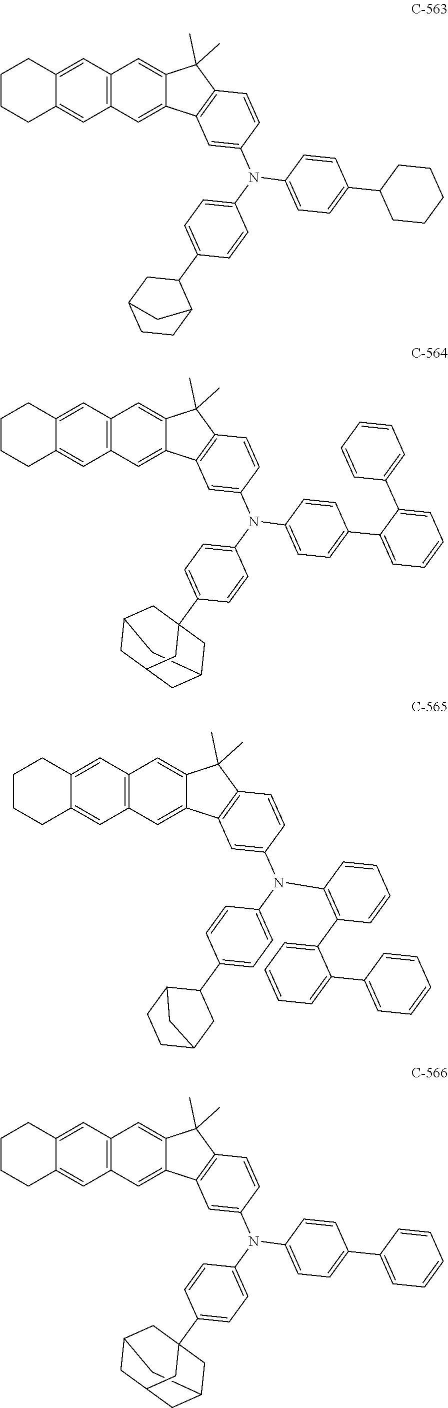 [SMILES: CC1(C)c2ccc(N(c3ccc(C4CCCCC4)cc3)c3ccc(C4CC5CCC4C5)cc3)cc2-c2cc3cc4c(cc3cc21)CCCC4 ; CC1(C)c2ccc(N(c3ccc(-c4ccccc4-c4ccccc4)cc3)c3ccc(C45CC6CC(CC(C6)C4)C5)cc3)cc2-c2cc3cc4c(cc3cc21)CCCC4 ; CC1(C)c2ccc(N(c3ccc(C4CC5CCC4C5)cc3)c3ccccc3-c3ccccc3-c3ccccc3)cc2-c2cc3cc4c(cc3cc21)CCCC4 ; CC1(C)c2ccc(N(c3ccc(-c4ccccc4)cc3)c3ccc(C45CC6CC(CC(C6)C4)C5)cc3)cc2-c2cc3cc4c(cc3cc21)CCCC4]

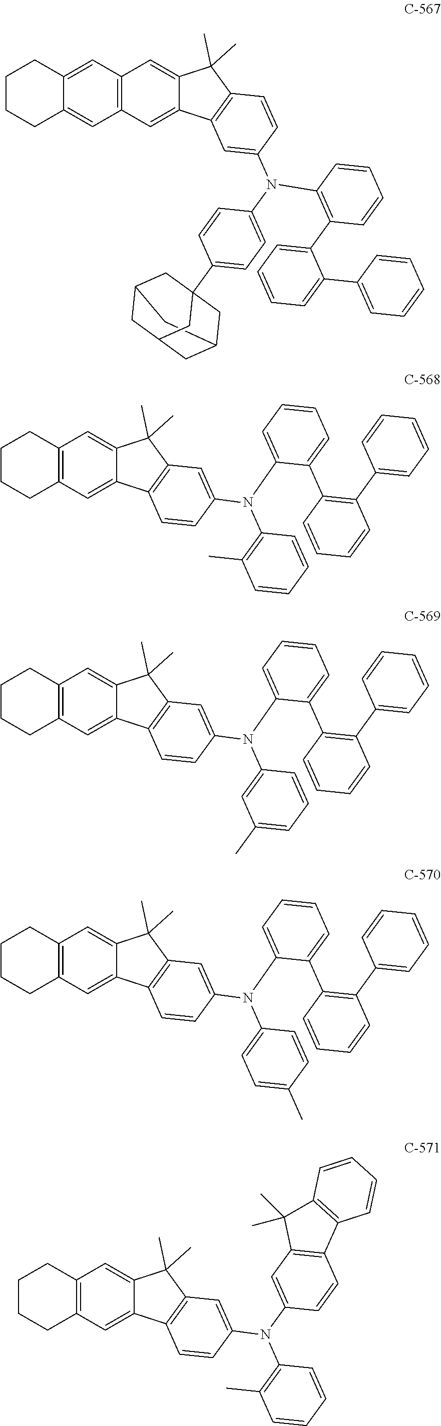 [SMILES: CC1(C)c2ccc(N(c3ccc(C45CC6CC(CC(C6)C4)C5)cc3)c3ccccc3-c3ccccc3-c3ccccc3)cc2-c2cc3cc4c(cc3cc21)CCCC4 ; Cc1ccccc1N(c1ccc2c(c1)C(C)(C)c1cc3c(cc1-2)CCCC3)c1ccccc1-c1ccccc1-c1ccccc1 ; Cc1cccc(N(c2ccc3c(c2)C(C)(C)c2cc4c(cc2-3)CCCC4)c2ccccc2-c2ccccc2-c2ccccc2)c1 ; Cc1ccc(N(c2ccc3c(c2)C(C)(C)c2cc4c(cc2-3)CCCC4)c2ccccc2-c2ccccc2-c2ccccc2)cc1 ; Cc1ccccc1N(c1ccc2c(c1)C(C)(C)c1ccccc1-2)c1ccc2c(c1)C(C)(C)c1cc3c(cc1-2)CCCC3]

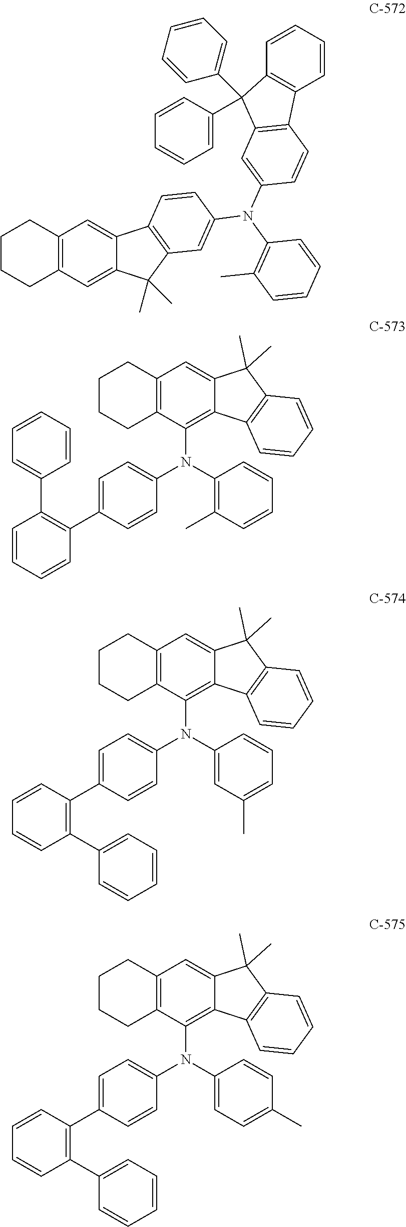

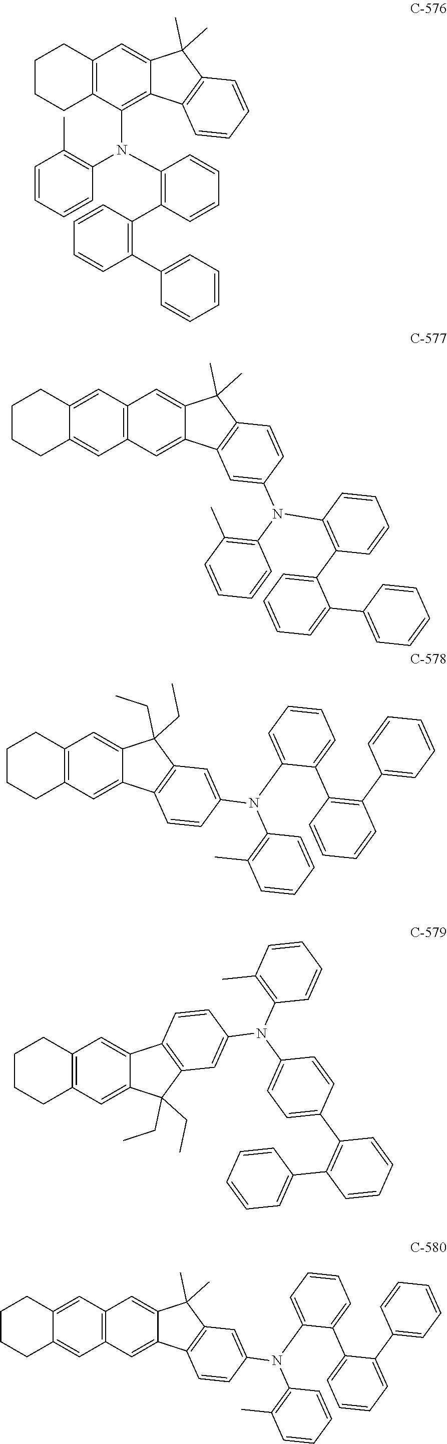

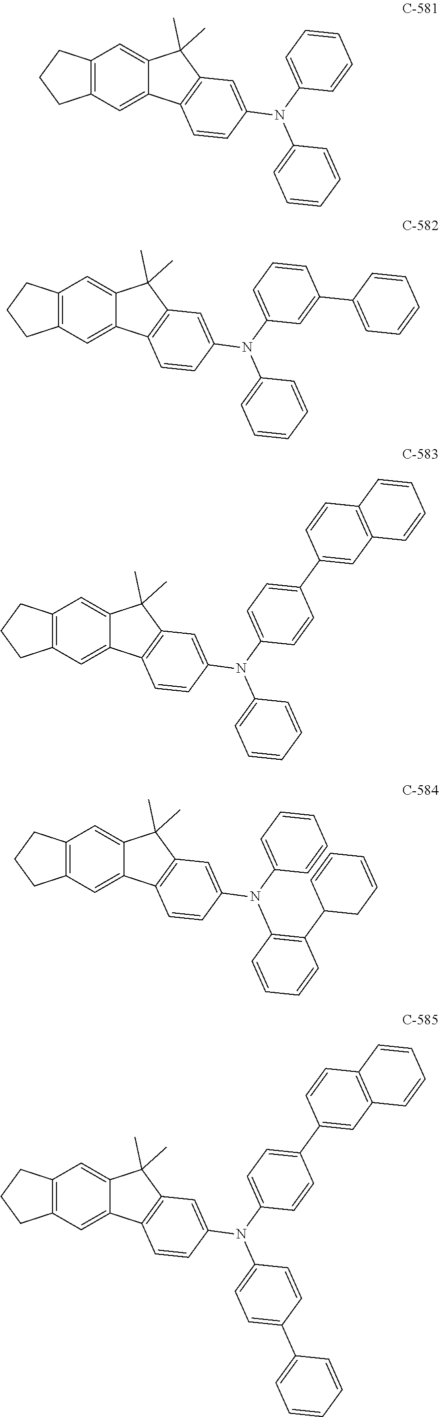 [SMILES: CC1(C)c2cc(N(c3ccccc3)c3ccccc3)ccc2-c2cc3c(cc21)CCC3 ; CC1(C)c2cc(N(c3ccccc3)c3cccc(-c4ccccc4)c3)ccc2-c2cc3c(cc21)CCC3 ; CC1(C)c2cc(N(c3ccccc3)c3ccc(-c4ccc5ccccc5c4)cc3)ccc2-c2cc3c(cc21)CCC3 ; CC1(C)c2cc(N(c3ccccc3)c3ccccc3C3C=CC=CC3)ccc2-c2cc3c(cc21)CCC3 ; CC1(C)c2cc(N(c3ccc(-c4ccccc4)cc3)c3ccc(-c4ccc5ccccc5c4)cc3)ccc2-c2cc3c(cc21)CCC3]

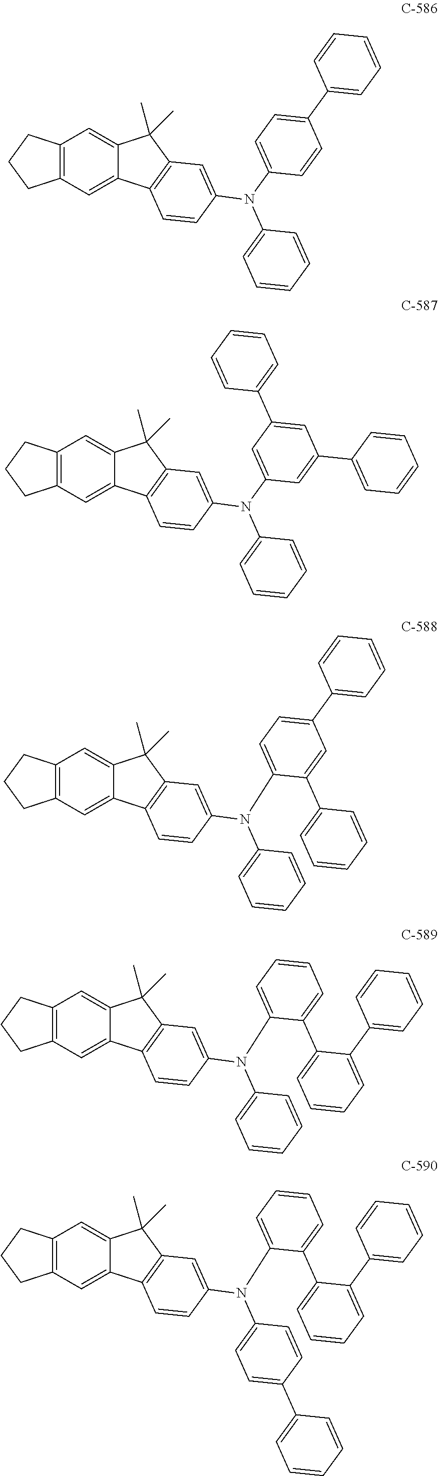

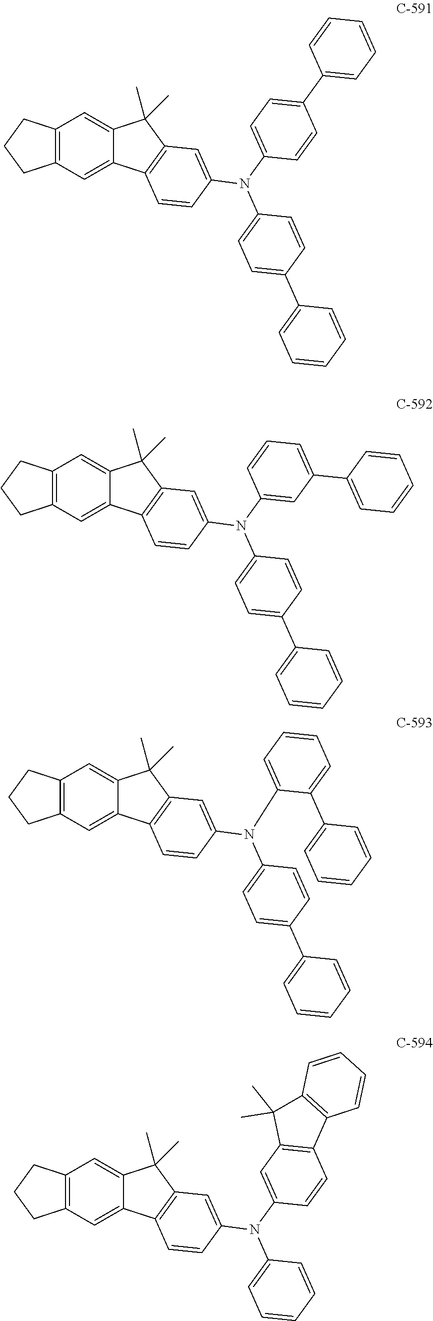

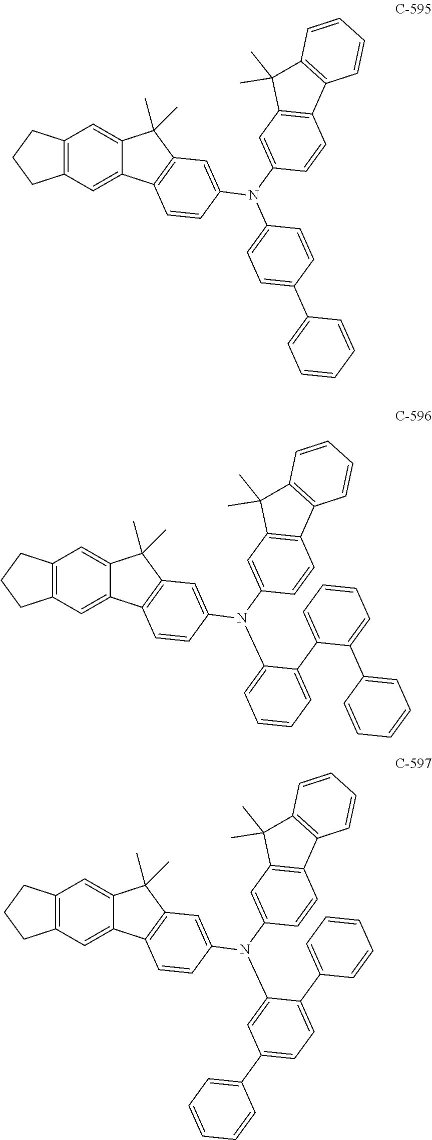 [SMILES: CC1(C)c2ccccc2-c2ccc(N(c3ccc(-c4ccccc4)cc3)c3ccc4c(c3)C(C)(C)c3cc5c(cc3-4)CCC5)cc21 ; CC1(C)c2ccccc2-c2ccc(N(c3ccc4c(c3)C(C)(C)c3cc5c(cc3-4)CCC5)c3ccccc3-c3ccccc3-c3ccccc3)cc21 ; CC1(C)c2ccccc2-c2ccc(N(c3ccc4c(c3)C(C)(C)c3cc5c(cc3-4)CCC5)c3cc(-c4ccccc4)ccc3-c3ccccc3)cc21]

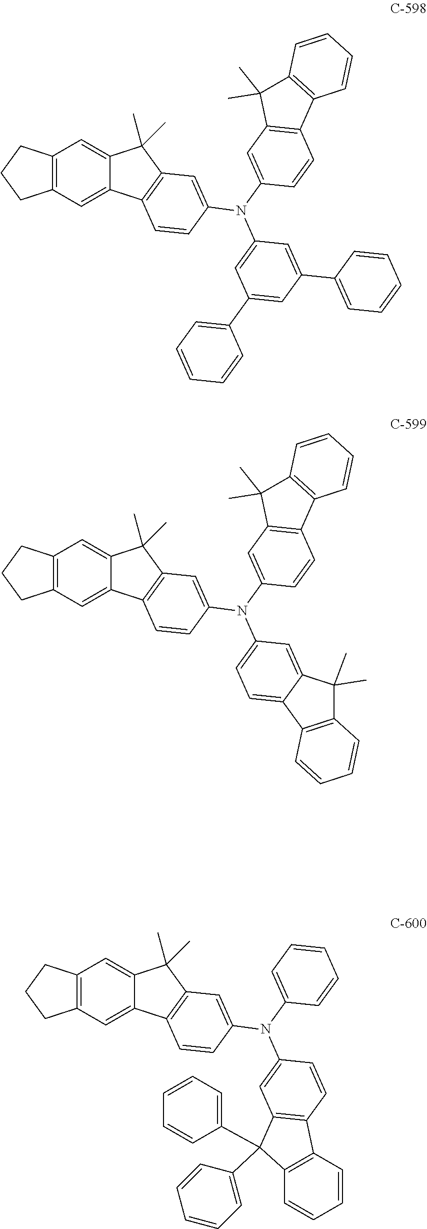 [SMILES: CC1(C)c2ccccc2-c2ccc(N(c3cc(-c4ccccc4)cc(-c4ccccc4)c3)c3ccc4c(c3)C(C)(C)c3cc5c(cc3-4)CCC5)cc21 ; CC1(C)c2ccccc2-c2ccc(N(c3ccc4c(c3)C(C)(C)c3ccccc3-4)c3ccc4c(c3)C(C)(C)c3cc5c(cc3-4)CCC5)cc21 ; CC1(C)c2cc(N(c3ccccc3)c3ccc4c(c3)C(c3ccccc3)(c3ccccc3)c3ccccc3-4)ccc2-c2cc3c(cc21)CCC3]

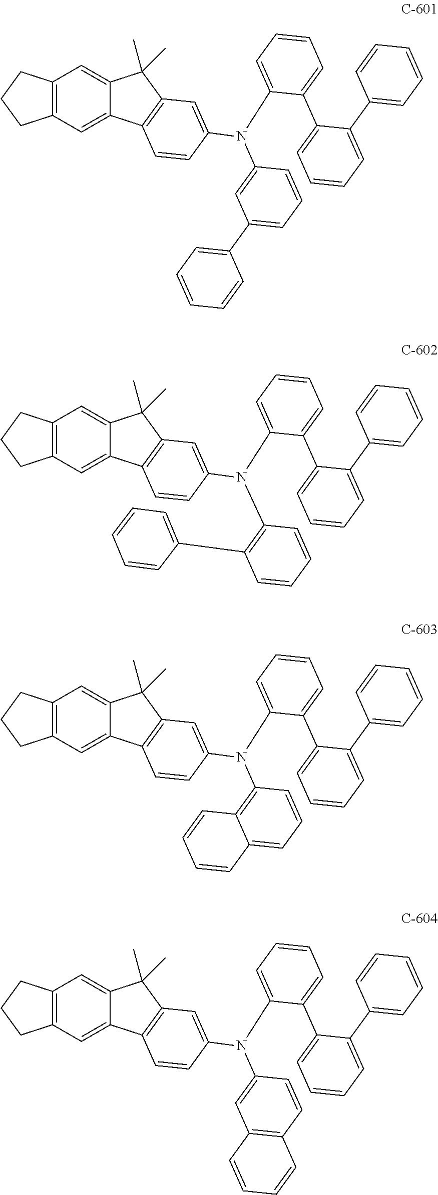 [SMILES: CC1(C)c2cc(N(c3cccc(-c4ccccc4)c3)c3ccccc3-c3ccccc3-c3ccccc3)ccc2-c2cc3c(cc21)CCC3 ; CC1(C)c2cc(N(c3ccccc3-c3ccccc3)c3ccccc3-c3ccccc3-c3ccccc3)ccc2-c2cc3c(cc21)CCC3 ; CC1(C)c2cc(N(c3ccccc3-c3ccccc3-c3ccccc3)c3cccc4ccccc34)ccc2-c2cc3c(cc21)CCC3 ; CC1(C)c2cc(N(c3ccc4ccccc4c3)c3ccccc3-c3ccccc3-c3ccccc3)ccc2-c2cc3c(cc21)CCC3]

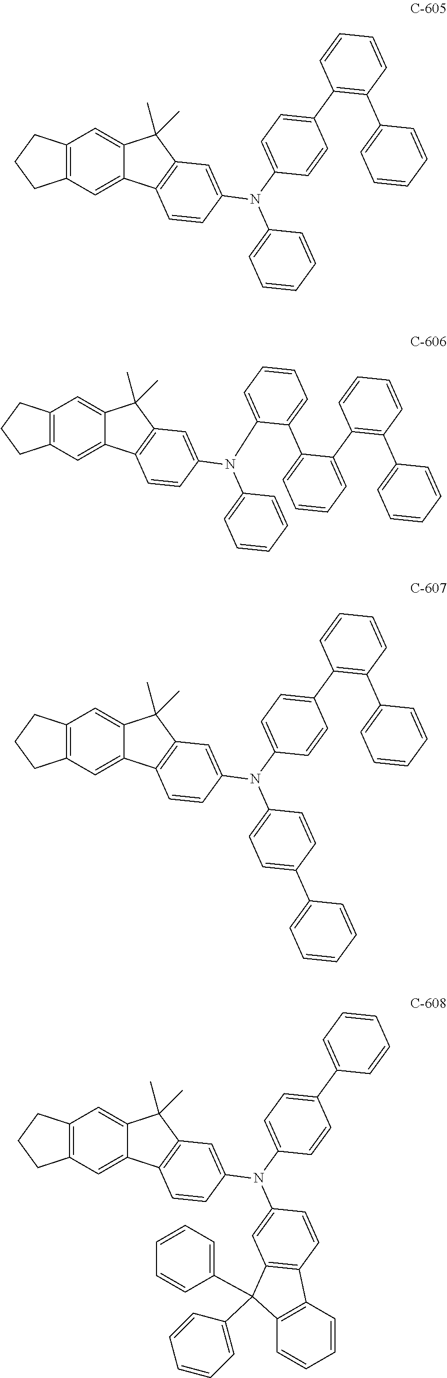 [SMILES: CC1(C)c2cc(N(c3ccccc3)c3ccc(-c4ccccc4-c4ccccc4)cc3)ccc2-c2cc3c(cc21)CCC3 ; CC1(C)c2cc(N(c3ccccc3)c3ccccc3-c3ccccc3-c3ccccc3-c3ccccc3)ccc2-c2cc3c(cc21)CCC3 ; CC1(C)c2cc(N(c3ccc(-c4ccccc4)cc3)c3ccc(-c4ccccc4-c4ccccc4)cc3)ccc2-c2cc3c(cc21)CCC3 ; CC1(C)c2cc(N(c3ccc(-c4ccccc4)cc3)c3ccc4c(c3)C(c3ccccc3)(c3ccccc3)c3ccccc3-4)ccc2-c2cc3c(cc21)CCC3]

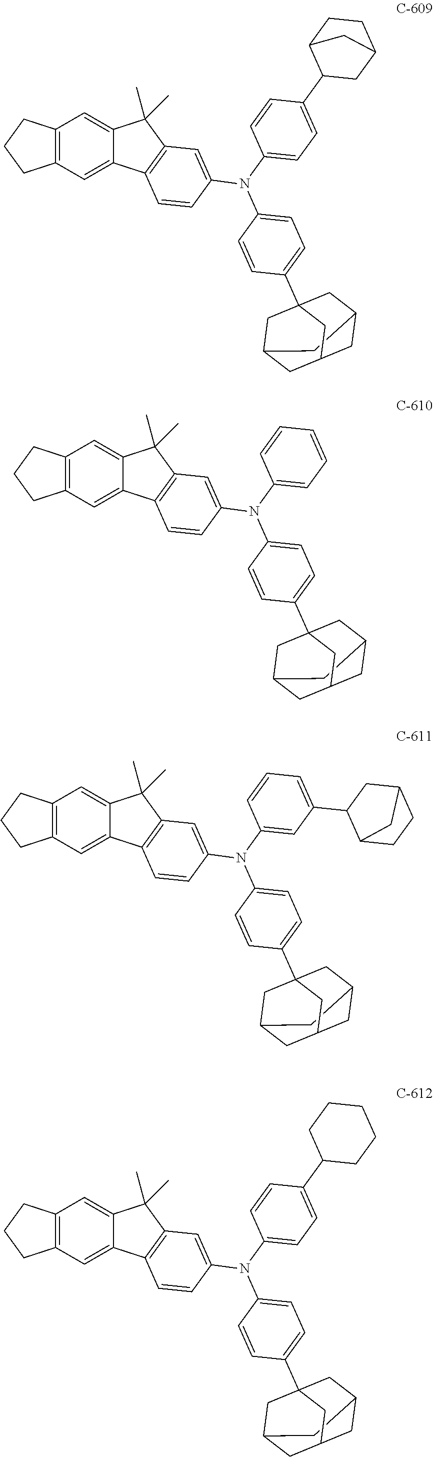 [SMILES: CC1(C)c2cc(N(c3ccc(C4CC5CCC4C5)cc3)c3ccc(C45CC6CC(CC(C6)C4)C5)cc3)ccc2-c2cc3c(cc21)CCC3 ; CC1(C)c2cc(N(c3ccccc3)c3ccc(C45CC6CC(CC(C6)C4)C5)cc3)ccc2-c2cc3c(cc21)CCC3 ; CC1(C)c2cc(N(c3ccc(C45CC6CC(CC(C6)C4)C5)cc3)c3cccc(C4CC5CCC4C5)c3)ccc2-c2cc3c(cc21)CCC3 ; CC1(C)c2cc(N(c3ccc(C4CCCCC4)cc3)c3ccc(C45CC6CC(CC(C6)C4)C5)cc3)ccc2-c2cc3c(cc21)CCC3]

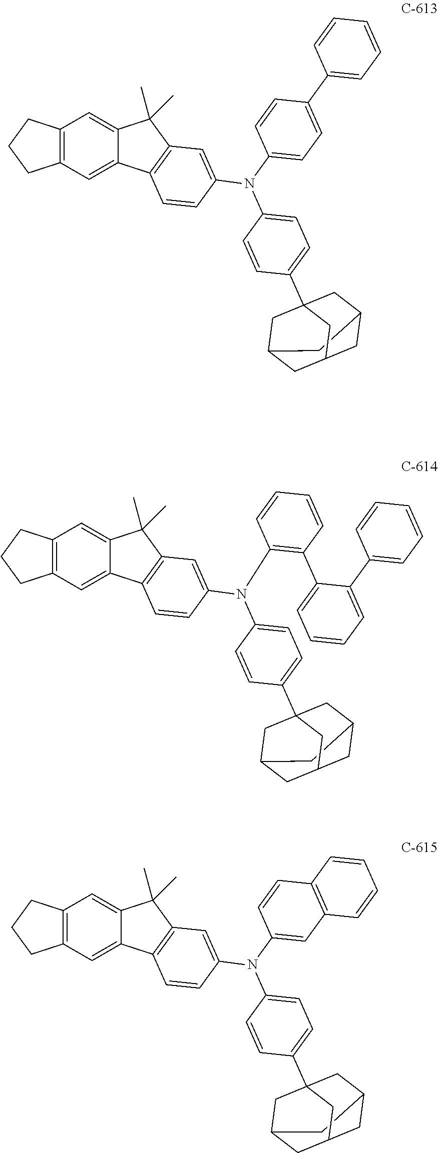 [SMILES: CC1(C)c2cc(N(c3ccc(-c4ccccc4)cc3)c3ccc(C45CC6CC(CC(C6)C4)C5)cc3)ccc2-c2cc3c(cc21)CCC3 ; CC1(C)c2cc(N(c3ccc(C45CC6CC(CC(C6)C4)C5)cc3)c3ccccc3-c3ccccc3-c3ccccc3)ccc2-c2cc3c(cc21)CCC3 ; CC1(C)c2cc(N(c3ccc(C45CC6CC(CC(C6)C4)C5)cc3)c3ccc4ccccc4c3)ccc2-c2cc3c(cc21)CCC3]

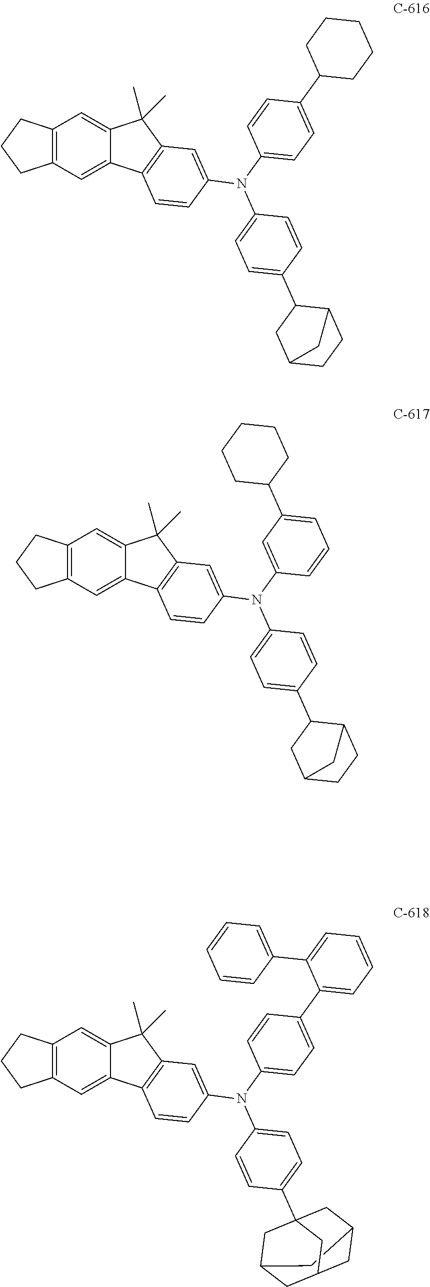 [SMILES: CC1(C)c2cc(N(c3ccc(C4CCCCC4)cc3)c3ccc(C4CC5CCC4C5)cc3)ccc2-c2cc3c(cc21)CCC3 ; CC1(C)c2cc(N(c3ccc(C4CC5CCC4C5)cc3)c3cccc(C4CCCCC4)c3)ccc2-c2cc3c(cc21)CCC3 ; CC1(C)c2cc(N(c3ccc(-c4ccccc4-c4ccccc4)cc3)c3ccc(C45CC6CC(CC(C6)C4)C5)cc3)ccc2-c2cc3c(cc21)CCC3]

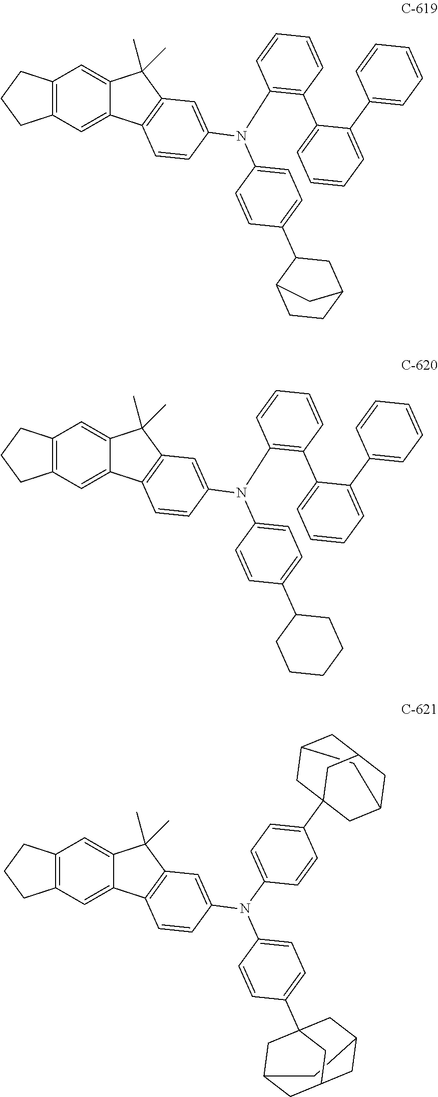 [SMILES: CC1(C)c2cc(N(c3ccc(C4CC5CCC4C5)cc3)c3ccccc3-c3ccccc3-c3ccccc3)ccc2-c2cc3c(cc21)CCC3 ; CC1(C)c2cc(N(c3ccc(C4CCCCC4)cc3)c3ccccc3-c3ccccc3-c3ccccc3)ccc2-c2cc3c(cc21)CCC3 ; CC1(C)c2cc(N(c3ccc(C45CC6CC(CC(C6)C4)C5)cc3)c3ccc(C45CC6CC(CC(C6)C4)C5)cc3)ccc2-c2cc3c(cc21)CCC3]

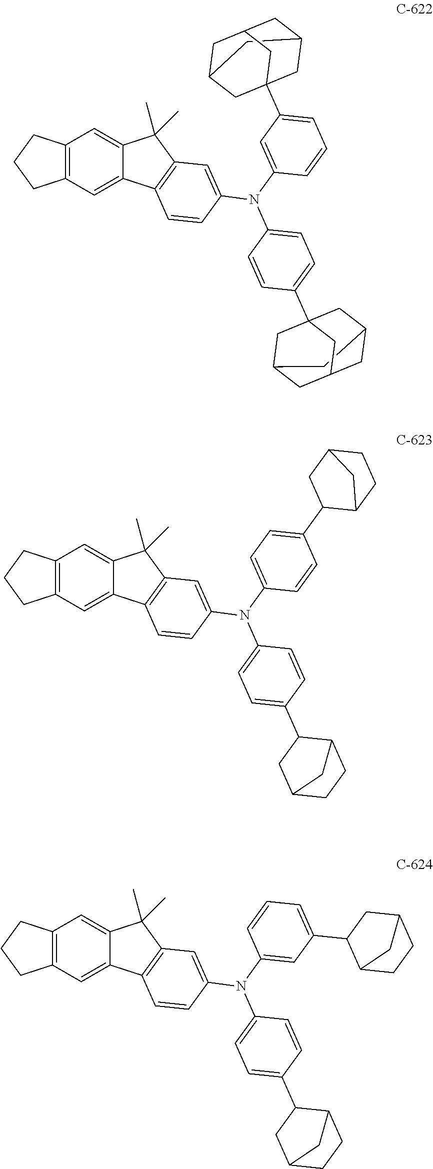 [SMILES: CC1(C)c2cc(N(c3ccc(C45CC6CC(CC(C6)C4)C5)cc3)c3cccc(C45CC6CC(CC(C6)C4)C5)c3)ccc2-c2cc3c(cc21)CCC3 ; CC1(C)c2cc(N(c3ccc(C4CC5CCC4C5)cc3)c3ccc(C4CC5CCC4C5)cc3)ccc2-c2cc3c(cc21)CCC3 ; CC1(C)c2cc(N(c3ccc(C4CC5CCC4C5)cc3)c3cccc(C4CC5CCC4C5)c3)ccc2-c2cc3c(cc21)CCC3]

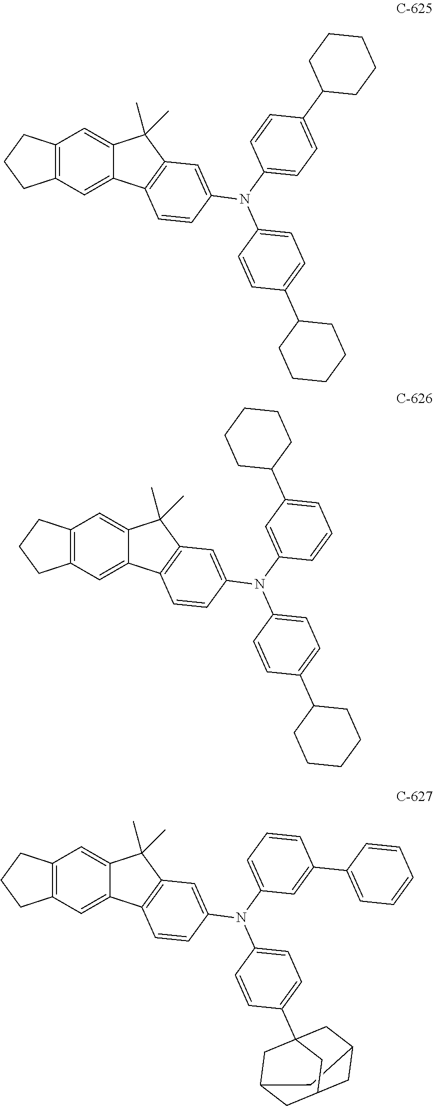

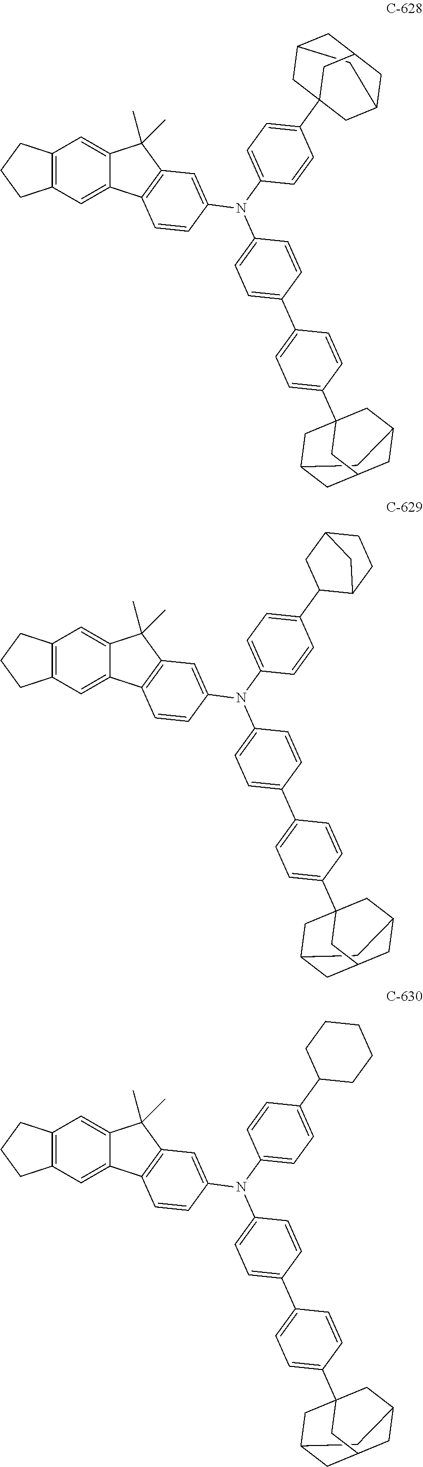

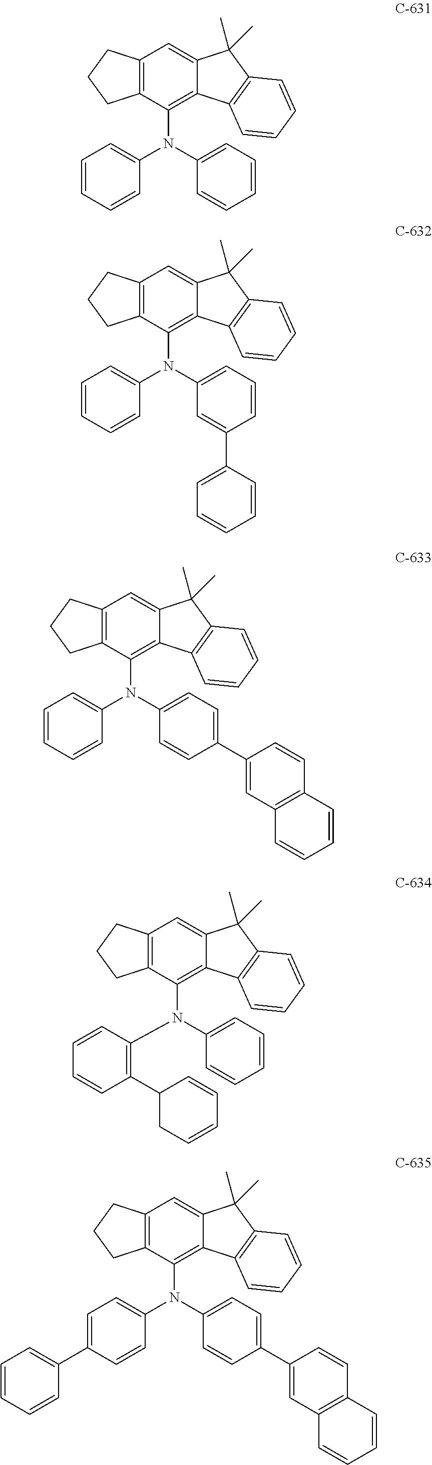

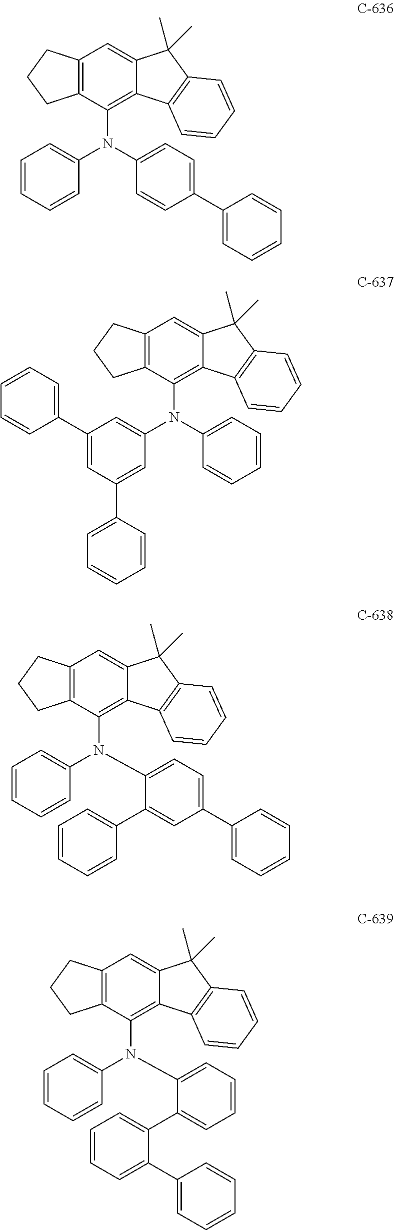

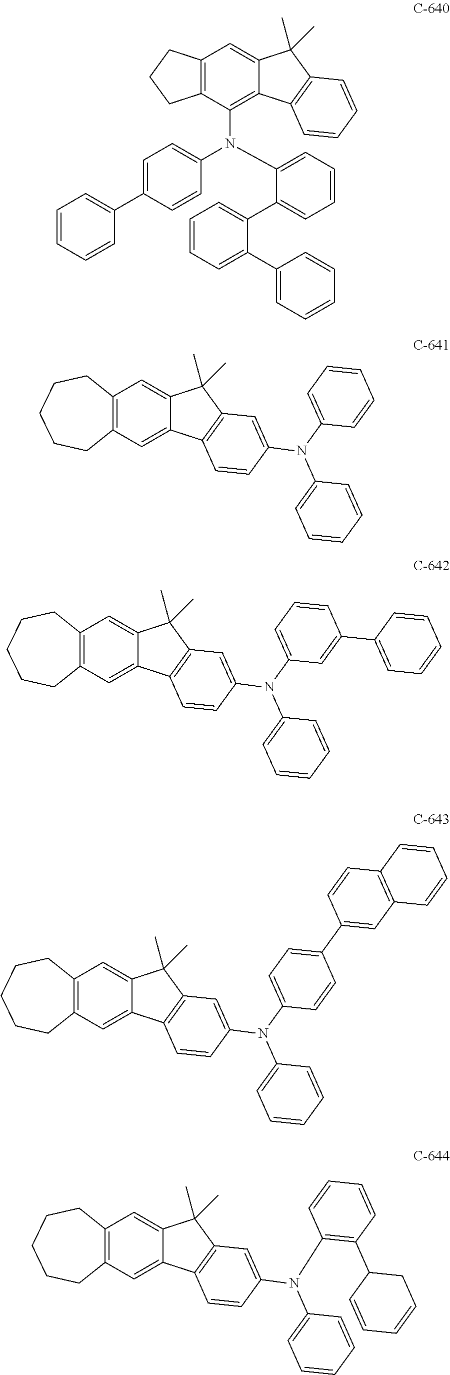 [SMILES: CC1(C)c2ccccc2-c2c1cc1c(c2N(c2ccc(-c3ccccc3)cc2)c2ccccc2-c2ccccc2-c2ccccc2)CCC1 ; CC1(C)c2cc(N(c3ccccc3)c3ccccc3)ccc2-c2cc3c(cc21)CCCCC3 ; CC1(C)c2cc(N(c3ccccc3)c3cccc(-c4ccccc4)c3)ccc2-c2cc3c(cc21)CCCCC3 ; CC1(C)c2cc(N(c3ccccc3)c3ccc(-c4ccc5ccccc5c4)cc3)ccc2-c2cc3c(cc21)CCCCC3 ; CC1(C)c2cc(N(c3ccccc3)c3ccccc3C3C=CC=CC3)ccc2-c2cc3c(cc21)CCCCC3]

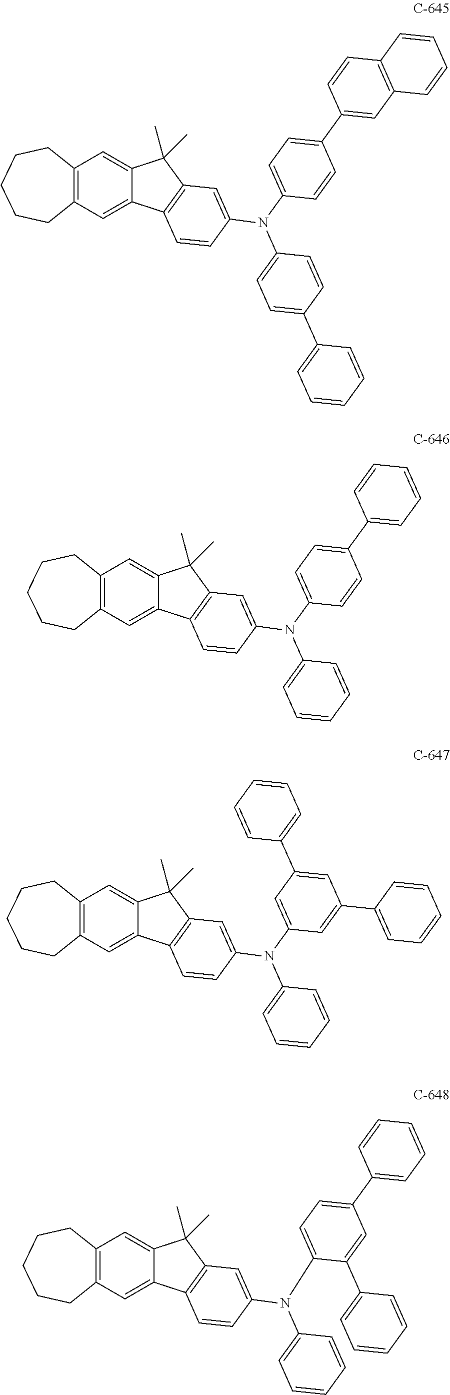 [SMILES: CC1(C)c2cc(N(c3ccc(-c4ccccc4)cc3)c3ccc(-c4ccc5ccccc5c4)cc3)ccc2-c2cc3c(cc21)CCCCC3 ; CC1(C)c2cc(N(c3ccccc3)c3ccc(-c4ccccc4)cc3)ccc2-c2cc3c(cc21)CCCCC3 ; CC1(C)c2cc(N(c3ccccc3)c3cc(-c4ccccc4)cc(-c4ccccc4)c3)ccc2-c2cc3c(cc21)CCCCC3 ; CC1(C)c2cc(N(c3ccccc3)c3ccc(-c4ccccc4)cc3-c3ccccc3)ccc2-c2cc3c(cc21)CCCCC3]

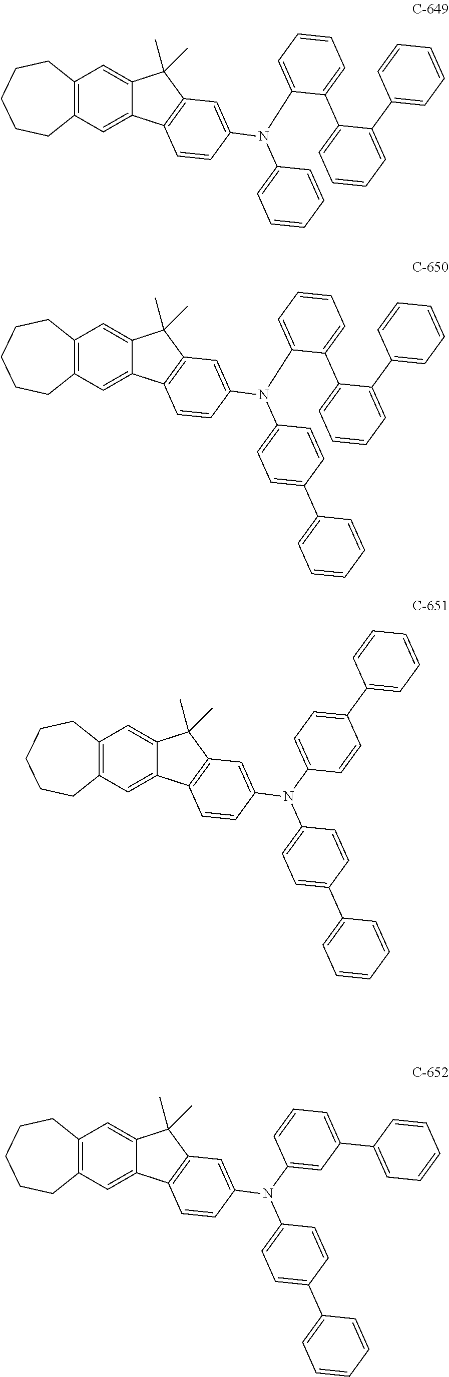 [SMILES: CC1(C)c2cc(N(c3ccccc3)c3ccccc3-c3ccccc3-c3ccccc3)ccc2-c2cc3c(cc21)CCCCC3 ; CC1(C)c2cc(N(c3ccc(-c4ccccc4)cc3)c3ccccc3-c3ccccc3-c3ccccc3)ccc2-c2cc3c(cc21)CCCCC3 ; CC1(C)c2cc(N(c3ccc(-c4ccccc4)cc3)c3ccc(-c4ccccc4)cc3)ccc2-c2cc3c(cc21)CCCCC3 ; CC1(C)c2cc(N(c3ccc(-c4ccccc4)cc3)c3cccc(-c4ccccc4)c3)ccc2-c2cc3c(cc21)CCCCC3]

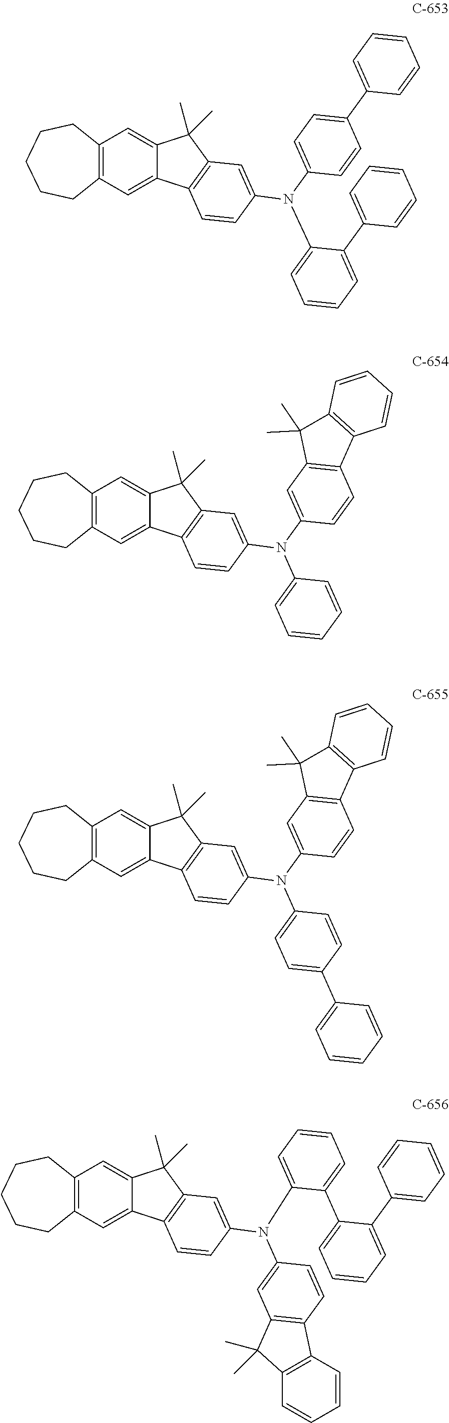 [SMILES: CC1(C)c2cc(N(c3ccc(-c4ccccc4)cc3)c3ccccc3-c3ccccc3)ccc2-c2cc3c(cc21)CCCCC3 ; CC1(C)c2ccccc2-c2ccc(N(c3ccccc3)c3ccc4c(c3)C(C)(C)c3cc5c(cc3-4)CCCCC5)cc21 ; CC1(C)c2ccccc2-c2ccc(N(c3ccc(-c4ccccc4)cc3)c3ccc4c(c3)C(C)(C)c3cc5c(cc3-4)CCCCC5)cc21 ; CC1(C)c2ccccc2-c2ccc(N(c3ccc4c(c3)C(C)(C)c3cc5c(cc3-4)CCCCC5)c3ccccc3-c3ccccc3-c3ccccc3)cc21]

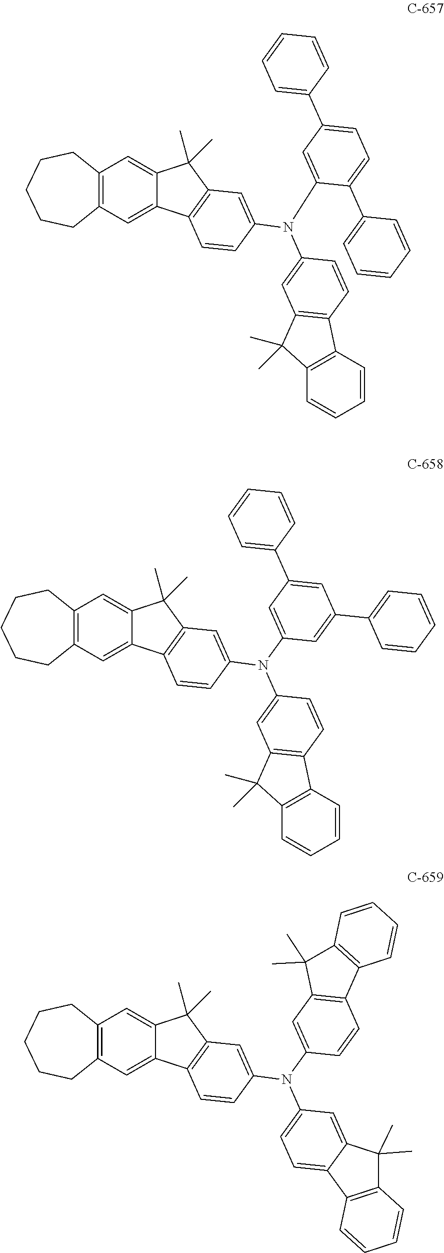 [SMILES: CC1(C)c2ccccc2-c2ccc(N(c3ccc4c(c3)C(C)(C)c3cc5c(cc3-4)CCCCC5)c3cc(-c4ccccc4)ccc3-c3ccccc3)cc21 ; CC1(C)c2ccccc2-c2ccc(N(c3cc(-c4ccccc4)cc(-c4ccccc4)c3)c3ccc4c(c3)C(C)(C)c3cc5c(cc3-4)CCCCC5)cc21 ; CC1(C)c2ccccc2-c2ccc(N(c3ccc4c(c3)C(C)(C)c3ccccc3-4)c3ccc4c(c3)C(C)(C)c3cc5c(cc3-4)CCCCC5)cc21]

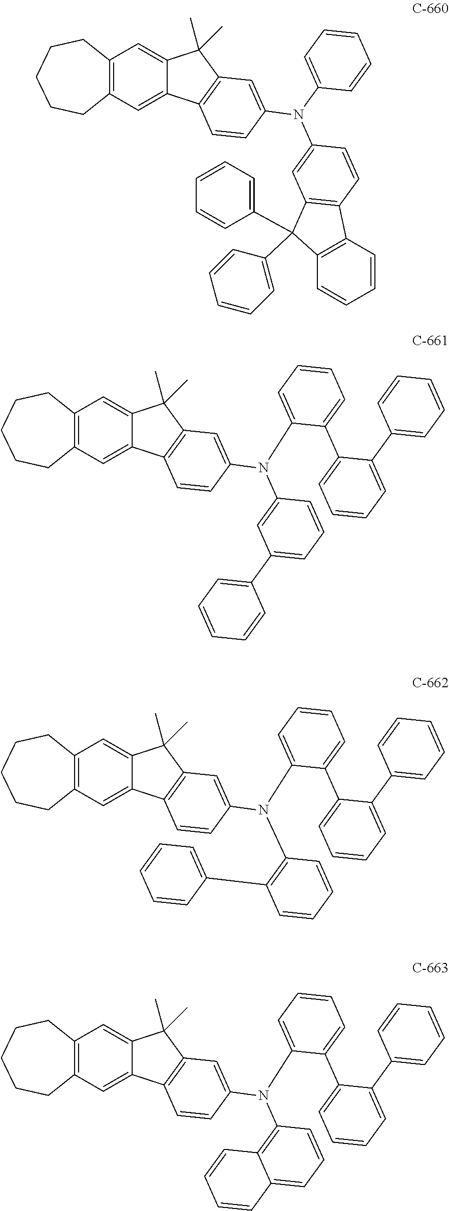 [SMILES: CC1(C)c2cc(N(c3ccccc3)c3ccc4c(c3)C(c3ccccc3)(c3ccccc3)c3ccccc3-4)ccc2-c2cc3c(cc21)CCCCC3 ; CC1(C)c2cc(N(c3cccc(-c4ccccc4)c3)c3ccccc3-c3ccccc3-c3ccccc3)ccc2-c2cc3c(cc21)CCCCC3 ; CC1(C)c2cc(N(c3ccccc3-c3ccccc3)c3ccccc3-c3ccccc3-c3ccccc3)ccc2-c2cc3c(cc21)CCCCC3 ; CC1(C)c2cc(N(c3ccccc3-c3ccccc3-c3ccccc3)c3cccc4ccccc34)ccc2-c2cc3c(cc21)CCCCC3]

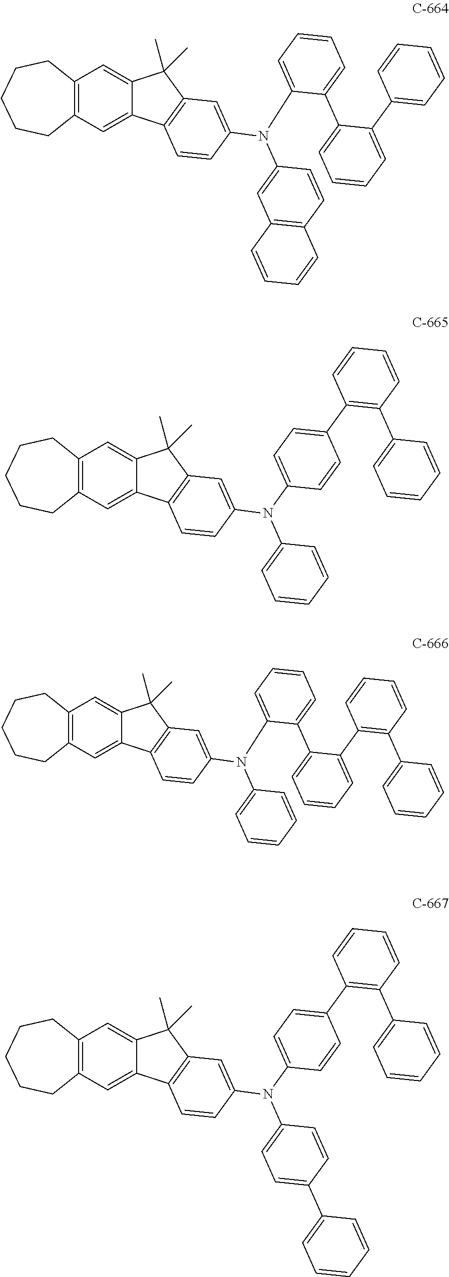 [SMILES: CC1(C)c2cc(N(c3ccc4ccccc4c3)c3ccccc3-c3ccccc3-c3ccccc3)ccc2-c2cc3c(cc21)CCCCC3 ; CC1(C)c2cc(N(c3ccccc3)c3ccc(-c4ccccc4-c4ccccc4)cc3)ccc2-c2cc3c(cc21)CCCCC3 ; CC1(C)c2cc(N(c3ccccc3)c3ccccc3-c3ccccc3-c3ccccc3-c3ccccc3)ccc2-c2cc3c(cc21)CCCCC3 ; CC1(C)c2cc(N(c3ccc(-c4ccccc4)cc3)c3ccc(-c4ccccc4-c4ccccc4)cc3)ccc2-c2cc3c(cc21)CCCCC3]

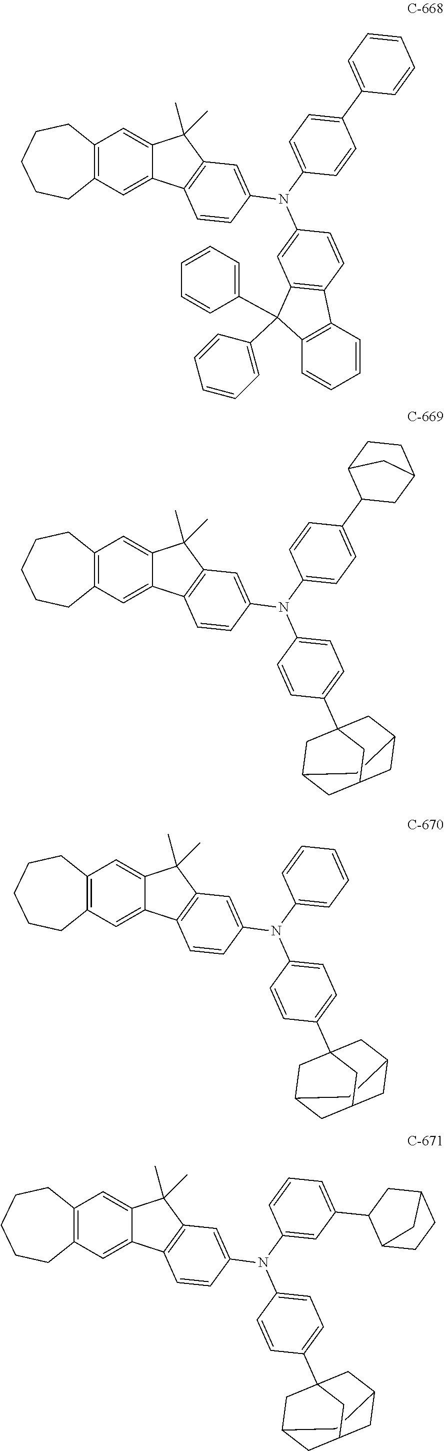 [SMILES: CC1(C)c2cc(N(c3ccc(-c4ccccc4)cc3)c3ccc4c(c3)C(c3ccccc3)(c3ccccc3)c3ccccc3-4)ccc2-c2cc3c(cc21)CCCCC3 ; CC1(C)c2cc(N(c3ccc(C4CC5CCC4C5)cc3)c3ccc(C45CC6CC(CC(C6)C4)C5)cc3)ccc2-c2cc3c(cc21)CCCCC3 ; CC1(C)c2cc(N(c3ccccc3)c3ccc(C45CC6CC(CC(C6)C4)C5)cc3)ccc2-c2cc3c(cc21)CCCCC3 ; CC1(C)c2cc(N(c3ccc(C45CC6CC(CC(C6)C4)C5)cc3)c3cccc(C4CC5CCC4C5)c3)ccc2-c2cc3c(cc21)CCCCC3]

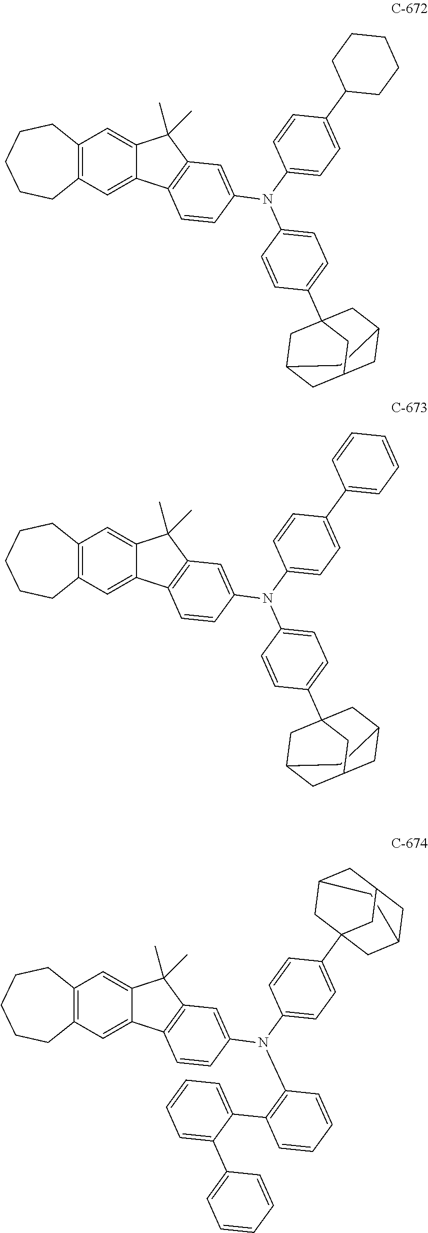 [SMILES: CC1(C)c2cc(N(c3ccc(C4CCCCC4)cc3)c3ccc(C45CC6CC(CC(C6)C4)C5)cc3)ccc2-c2cc3c(cc21)CCCCC3 ; CC1(C)c2cc(N(c3ccc(-c4ccccc4)cc3)c3ccc(C45CC6CC(CC(C6)C4)C5)cc3)ccc2-c2cc3c(cc21)CCCCC3 ; CC1(C)c2cc(N(c3ccc(C45CC6CC(CC(C6)C4)C5)cc3)c3ccccc3-c3ccccc3-c3ccccc3)ccc2-c2cc3c(cc21)CCCCC3]

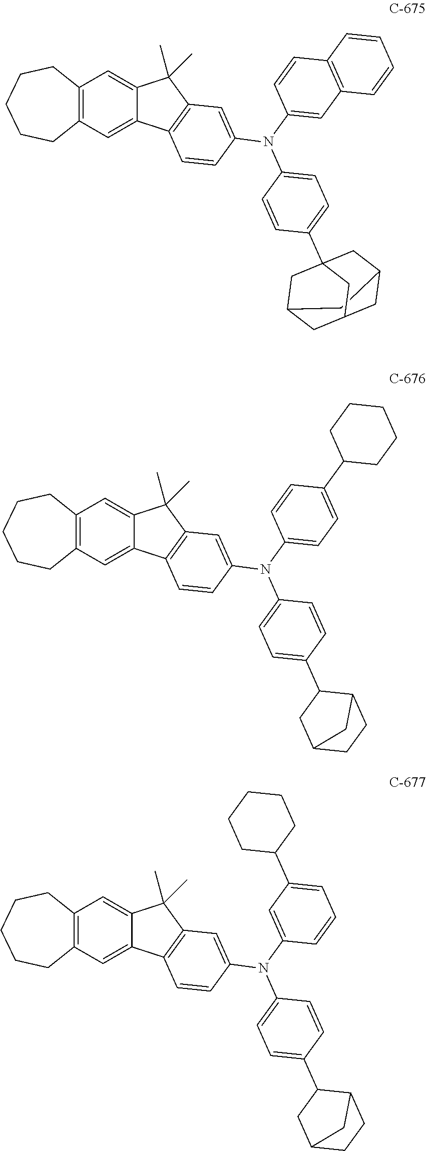 [SMILES: CC1(C)c2cc(N(c3ccc(C45CC6CC(CC(C6)C4)C5)cc3)c3ccc4ccccc4c3)ccc2-c2cc3c(cc21)CCCCC3 ; CC1(C)c2cc(N(c3ccc(C4CCCCC4)cc3)c3ccc(C4CC5CCC4C5)cc3)ccc2-c2cc3c(cc21)CCCCC3 ; CC1(C)c2cc(N(c3ccc(C4CC5CCC4C5)cc3)c3cccc(C4CCCCC4)c3)ccc2-c2cc3c(cc21)CCCCC3]

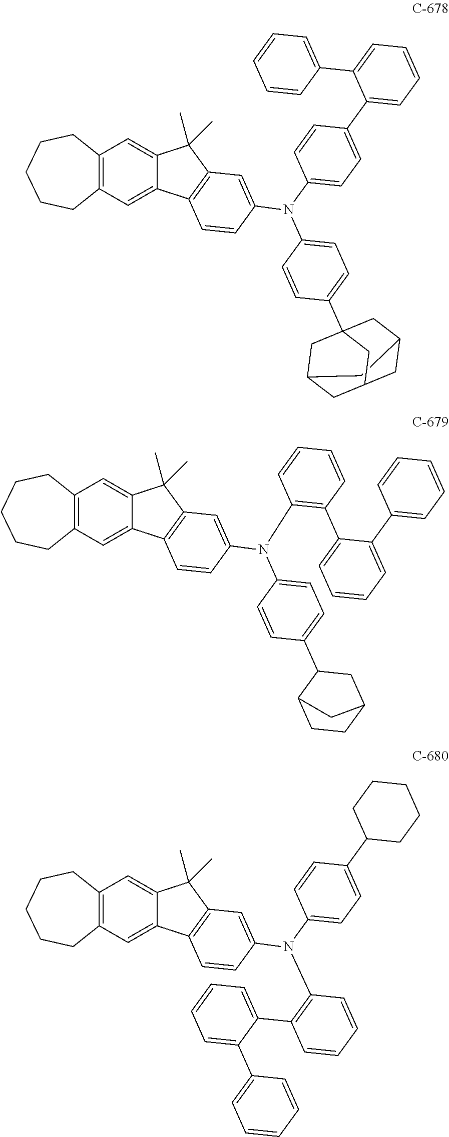 [SMILES: CC1(C)c2cc(N(c3ccc(-c4ccccc4-c4ccccc4)cc3)c3ccc(C45CC6CC(CC(C6)C4)C5)cc3)ccc2-c2cc3c(cc21)CCCCC3 ; CC1(C)c2cc(N(c3ccc(C4CC5CCC4C5)cc3)c3ccccc3-c3ccccc3-c3ccccc3)ccc2-c2cc3c(cc21)CCCCC3 ; CC1(C)c2cc(N(c3ccc(C4CCCCC4)cc3)c3ccccc3-c3ccccc3-c3ccccc3)ccc2-c2cc3c(cc21)CCCCC3]

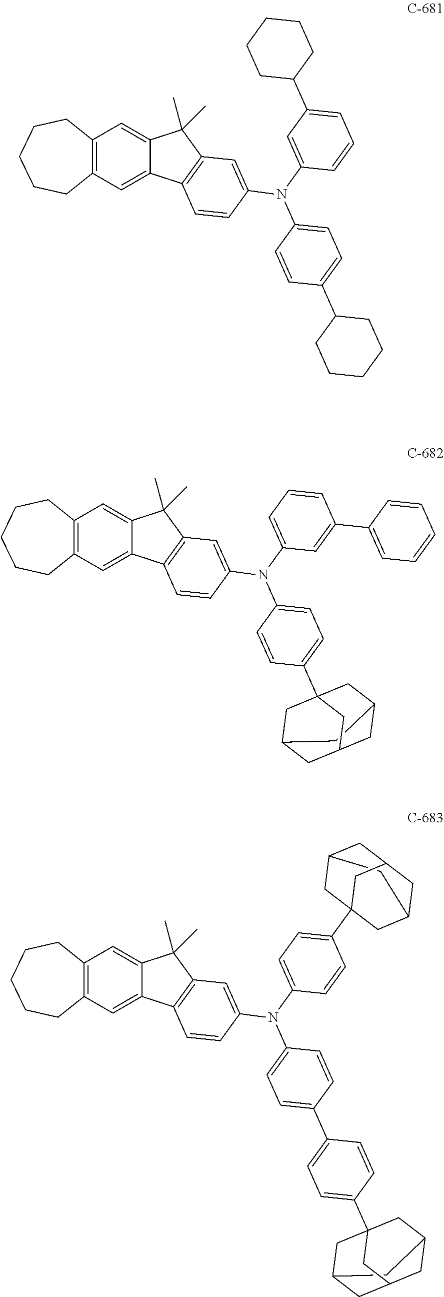 [SMILES: CC1(C)c2cc(N(c3ccc(C4CCCCC4)cc3)c3cccc(C4CCCCC4)c3)ccc2-c2cc3c(cc21)CCCCC3 ; CC1(C)c2cc(N(c3ccc(C45CC6CC(CC(C6)C4)C5)cc3)c3cccc(-c4ccccc4)c3)ccc2-c2cc3c(cc21)CCCCC3 ; CC1(C)c2cc(N(c3ccc(-c4ccc(C56CC7CC(CC(C7)C5)C6)cc4)cc3)c3ccc(C45CC6CC(CC(C6)C4)C5)cc3)ccc2-c2cc3c(cc21)CCCCC3]

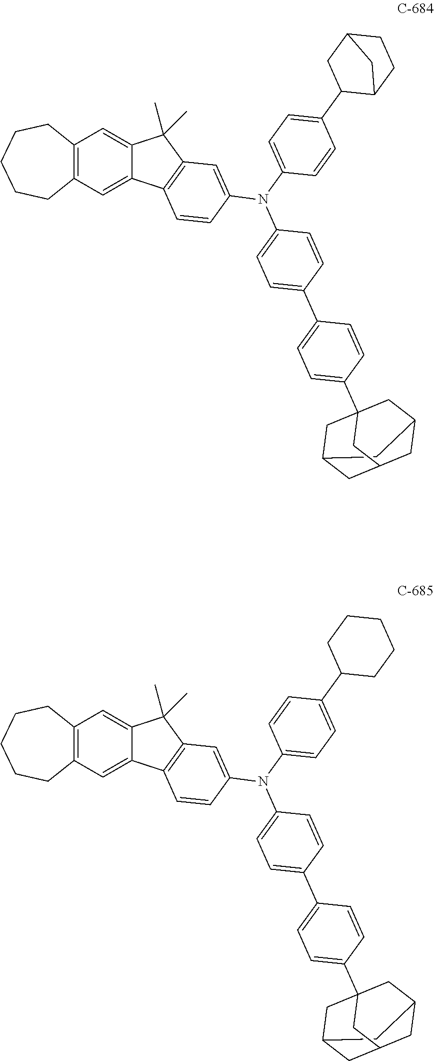 [SMILES: CC1(C)c2cc(N(c3ccc(-c4ccc(C56CC7CC(CC(C7)C5)C6)cc4)cc3)c3ccc(C4CC5CCC4C5)cc3)ccc2-c2cc3c(cc21)CCCCC3 ; CC1(C)c2cc(N(c3ccc(-c4ccc(C56CC7CC(CC(C7)C5)C6)cc4)cc3)c3ccc(C4CCCCC4)cc3)ccc2-c2cc3c(cc21)CCCCC3]

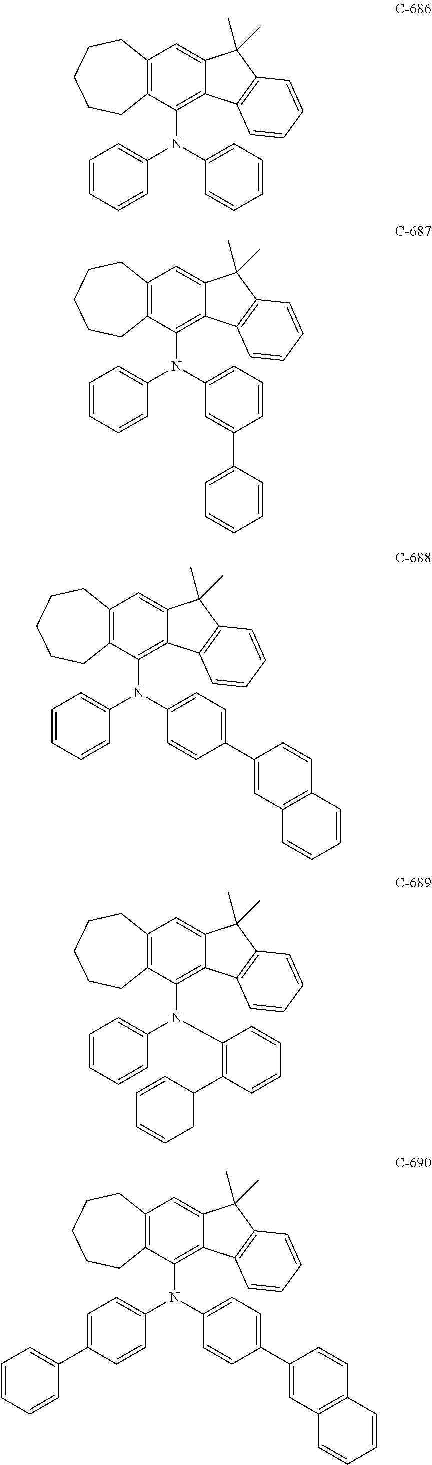

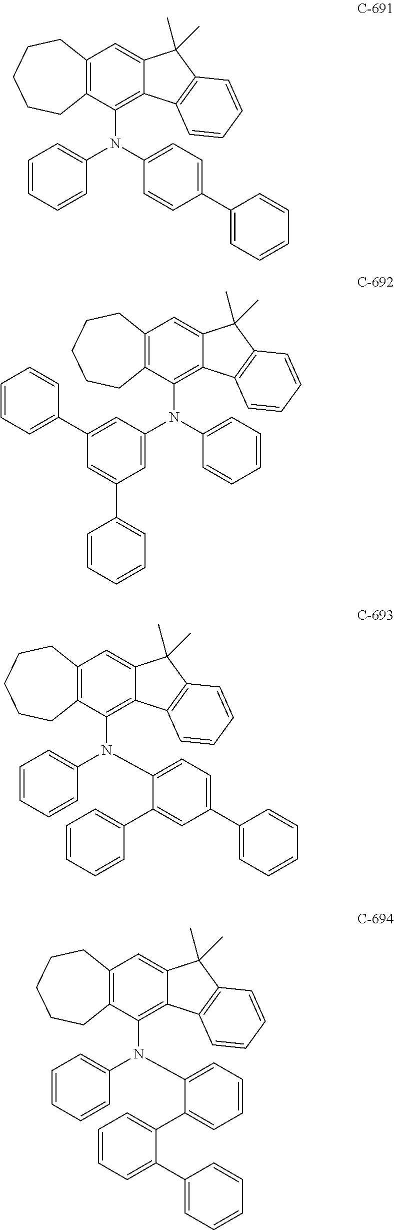

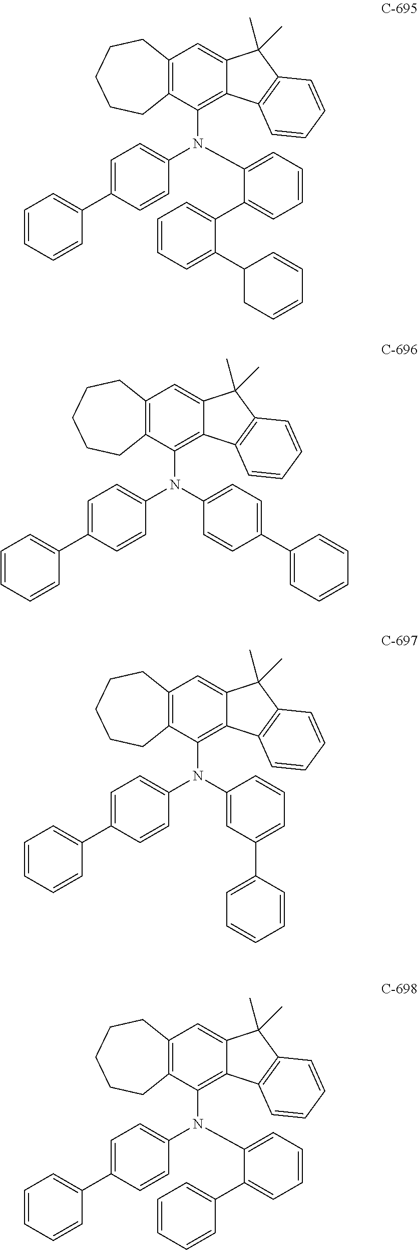 [SMILES: CC1(C)c2ccccc2-c2c1cc1c(c2N(c2ccc(-c3ccccc3)cc2)c2ccccc2-c2ccccc2C2C=CC=CC2)CCCCC1 ; CC1(C)c2ccccc2-c2c1cc1c(c2N(c2ccc(-c3ccccc3)cc2)c2ccc(-c3ccccc3)cc2)CCCCC1 ; CC1(C)c2ccccc2-c2c1cc1c(c2N(c2ccc(-c3ccccc3)cc2)c2cccc(-c3ccccc3)c2)CCCCC1 ; CC1(C)c2ccccc2-c2c1cc1c(c2N(c2ccc(-c3ccccc3)cc2)c2ccccc2-c2ccccc2)CCCCC1]

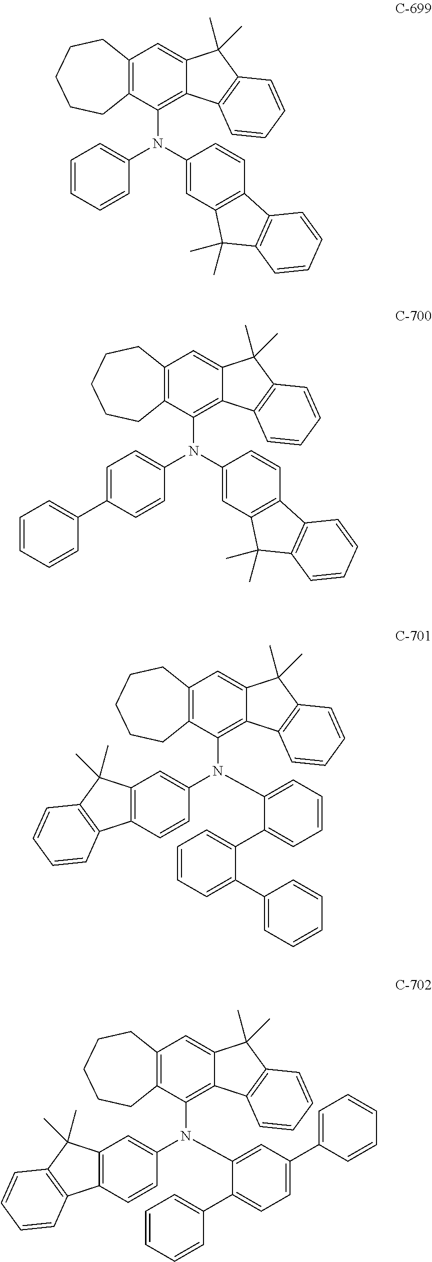 [SMILES: CC1(C)c2ccccc2-c2ccc(N(c3ccccc3)c3c4c(cc5c3-c3ccccc3C5(C)C)CCCCC4)cc21 ; CC1(C)c2ccccc2-c2ccc(N(c3ccc(-c4ccccc4)cc3)c3c4c(cc5c3-c3ccccc3C5(C)C)CCCCC4)cc21 ; CC1(C)c2ccccc2-c2ccc(N(c3ccccc3-c3ccccc3-c3ccccc3)c3c4c(cc5c3-c3ccccc3C5(C)C)CCCCC4)cc21 ; CC1(C)c2ccccc2-c2ccc(N(c3cc(-c4ccccc4)ccc3-c3ccccc3)c3c4c(cc5c3-c3ccccc3C5(C)C)CCCCC4)cc21]

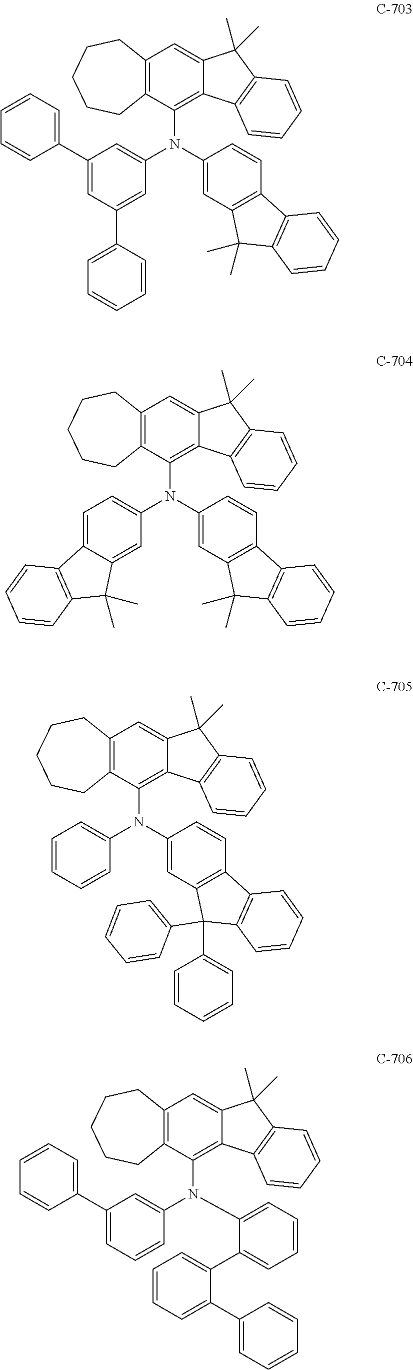 [SMILES: CC1(C)c2ccccc2-c2ccc(N(c3cc(-c4ccccc4)cc(-c4ccccc4)c3)c3c4c(cc5c3-c3ccccc3C5(C)C)CCCCC4)cc21 ; CC1(C)c2ccccc2-c2ccc(N(c3ccc4c(c3)C(C)(C)c3ccccc3-4)c3c4c(cc5c3-c3ccccc3C5(C)C)CCCCC4)cc21 ; CC1(C)c2ccccc2-c2c1cc1c(c2N(c2ccccc2)c2ccc3c(c2)C(c2ccccc2)(c2ccccc2)c2ccccc2-3)CCCCC1 ; CC1(C)c2ccccc2-c2c1cc1c(c2N(c2cccc(-c3ccccc3)c2)c2ccccc2-c2ccccc2-c2ccccc2)CCCCC1]

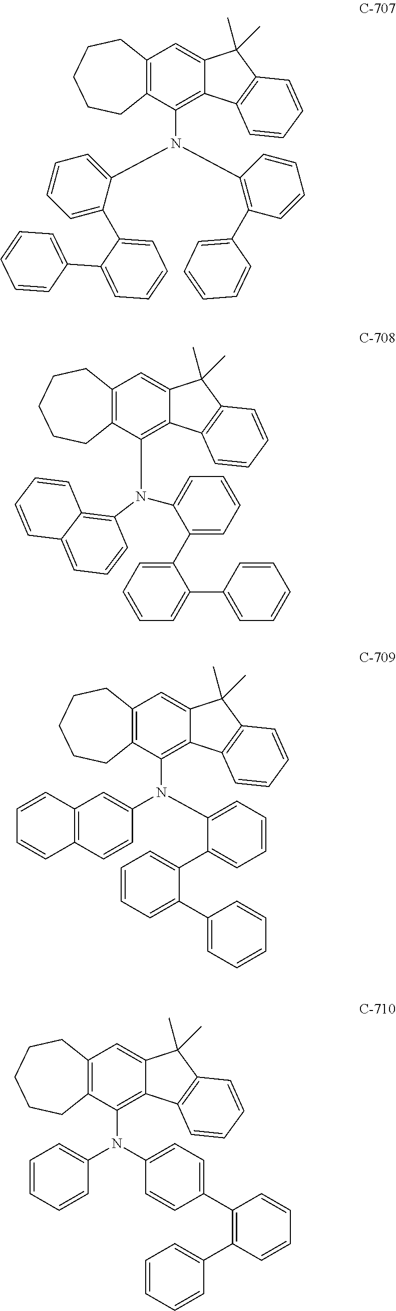 [SMILES: CC1(C)c2ccccc2-c2c1cc1c(c2N(c2ccccc2-c2ccccc2)c2ccccc2-c2ccccc2-c2ccccc2)CCCCC1 ; CC1(C)c2ccccc2-c2c1cc1c(c2N(c2ccccc2-c2ccccc2-c2ccccc2)c2cccc3ccccc23)CCCCC1 ; CC1(C)c2ccccc2-c2c1cc1c(c2N(c2ccc3ccccc3c2)c2ccccc2-c2ccccc2-c2ccccc2)CCCCC1 ; CC1(C)c2ccccc2-c2c1cc1c(c2N(c2ccccc2)c2ccc(-c3ccccc3-c3ccccc3)cc2)CCCCC1]

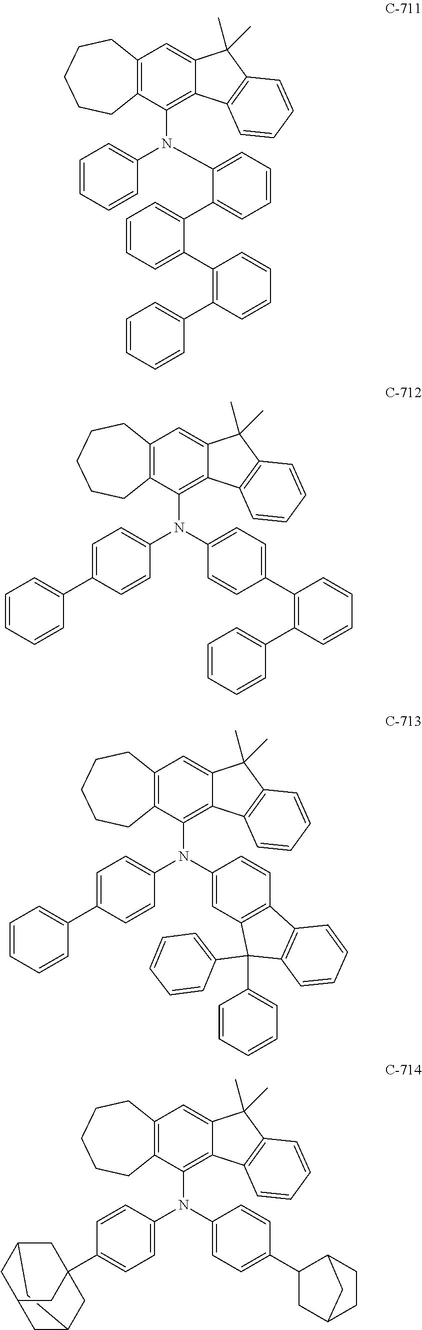

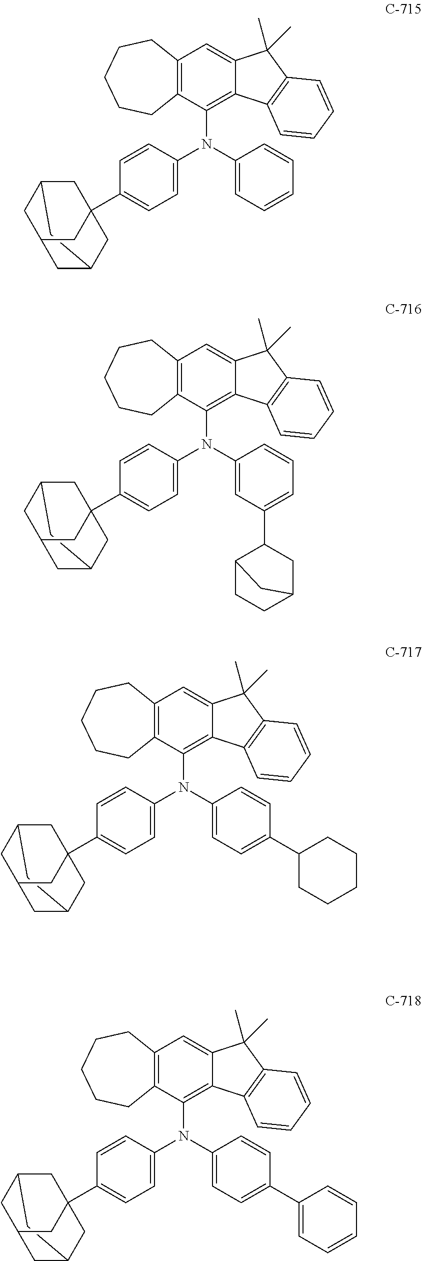 [SMILES: CC1(C)c2ccccc2-c2c1cc1c(c2N(c2ccccc2)c2ccc(C34CC5CC(CC(C5)C3)C4)cc2)CCCCC1 ; CC1(C)c2ccccc2-c2c1cc1c(c2N(c2ccc(C34CC5CC(CC(C5)C3)C4)cc2)c2cccc(C3CC4CCC3C4)c2)CCCCC1 ; CC1(C)c2ccccc2-c2c1cc1c(c2N(c2ccc(C3CCCCC3)cc2)c2ccc(C34CC5CC(CC(C5)C3)C4)cc2)CCCCC1 ; CC1(C)c2ccccc2-c2c1cc1c(c2N(c2ccc(-c3ccccc3)cc2)c2ccc(C34CC5CC(CC(C5)C3)C4)cc2)CCCCC1]

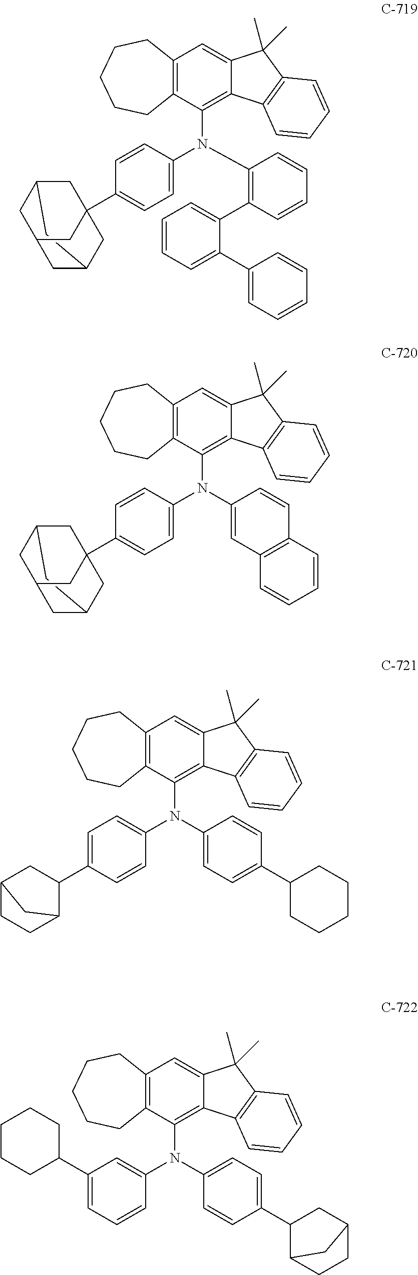 [SMILES: CC1(C)c2ccccc2-c2c1cc1c(c2N(c2ccc(C34CC5CC(CC(C5)C3)C4)cc2)c2ccccc2-c2ccccc2-c2ccccc2)CCCCC1 ; CC1(C)c2ccccc2-c2c1cc1c(c2N(c2ccc(C34CC5CC(CC(C5)C3)C4)cc2)c2ccc3ccccc3c2)CCCCC1 ; CC1(C)c2ccccc2-c2c1cc1c(c2N(c2ccc(C3CCCCC3)cc2)c2ccc(C3CC4CCC3C4)cc2)CCCCC1 ; CC1(C)c2ccccc2-c2c1cc1c(c2N(c2ccc(C3CC4CCC3C4)cc2)c2cccc(C3CCCCC3)c2)CCCCC1]

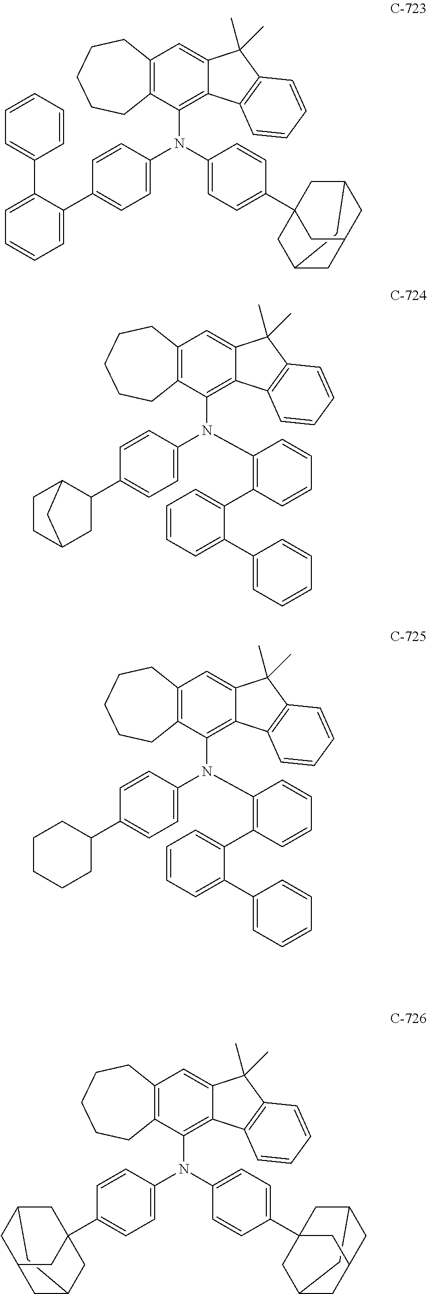

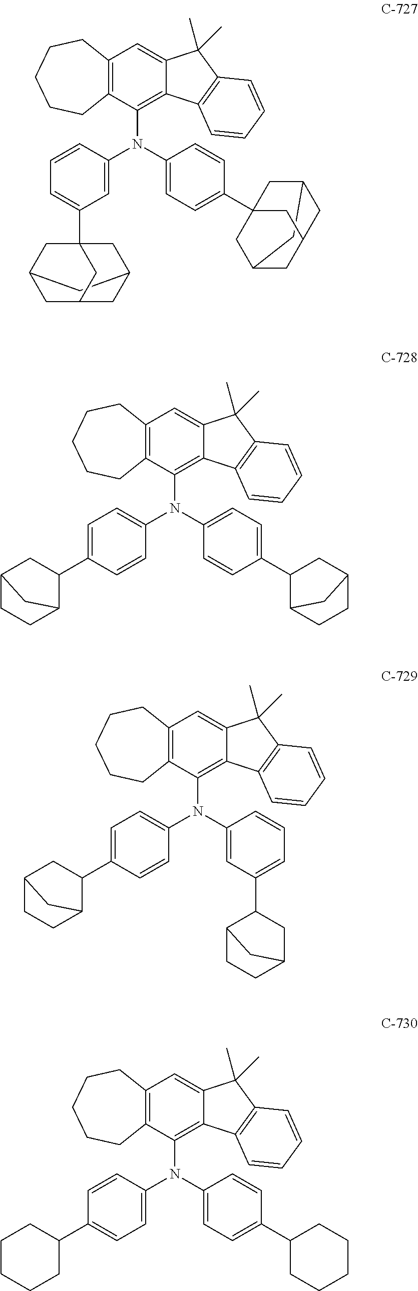 [SMILES: CC1(C)c2ccccc2-c2c1cc1c(c2N(c2ccc(C34CC5CC(CC(C5)C3)C4)cc2)c2cccc(C34CC5CC(CC(C5)C3)C4)c2)CCCCC1 ; CC1(C)c2ccccc2-c2c1cc1c(c2N(c2ccc(C3CC4CCC3C4)cc2)c2ccc(C3CC4CCC3C4)cc2)CCCCC1 ; CC1(C)c2ccccc2-c2c1cc1c(c2N(c2ccc(C3CC4CCC3C4)cc2)c2cccc(C3CC4CCC3C4)c2)CCCCC1 ; CC1(C)c2ccccc2-c2c1cc1c(c2N(c2ccc(C3CCCCC3)cc2)c2ccc(C3CCCCC3)cc2)CCCCC1]

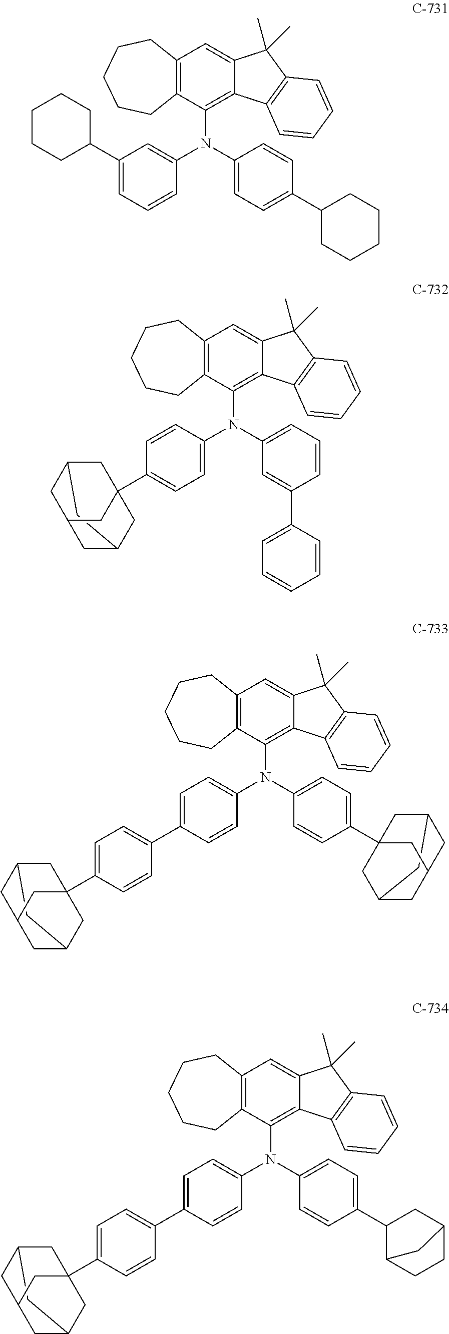 [SMILES: CC1(C)c2ccccc2-c2c1cc1c(c2N(c2ccc(C3CCCCC3)cc2)c2cccc(C3CCCCC3)c2)CCCCC1 ; CC1(C)c2ccccc2-c2c1cc1c(c2N(c2ccc(C34CC5CC(CC(C5)C3)C4)cc2)c2cccc(-c3ccccc3)c2)CCCCC1 ; CC1(C)c2ccccc2-c2c1cc1c(c2N(c2ccc(-c3ccc(C45CC6CC(CC(C6)C4)C5)cc3)cc2)c2ccc(C34CC5CC(CC(C5)C3)C4)cc2)CCCCC1 ; CC1(C)c2ccccc2-c2c1cc1c(c2N(c2ccc(-c3ccc(C45CC6CC(CC(C6)C4)C5)cc3)cc2)c2ccc(C3CC4CCC3C4)cc2)CCCCC1]

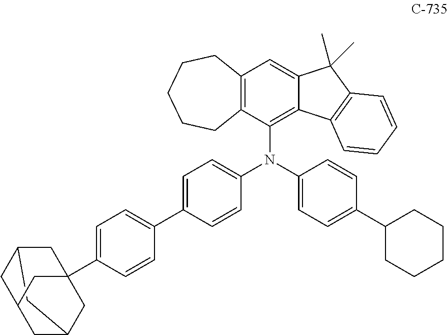 [SMILES: CC1(C)c2ccccc2-c2c1cc1c(c2N(c2ccc(-c3ccc(C45CC6CC(CC(C6)C4)C5)cc3)cc2)c2ccc(C3CCCCC3)cc2)CCCCC1]

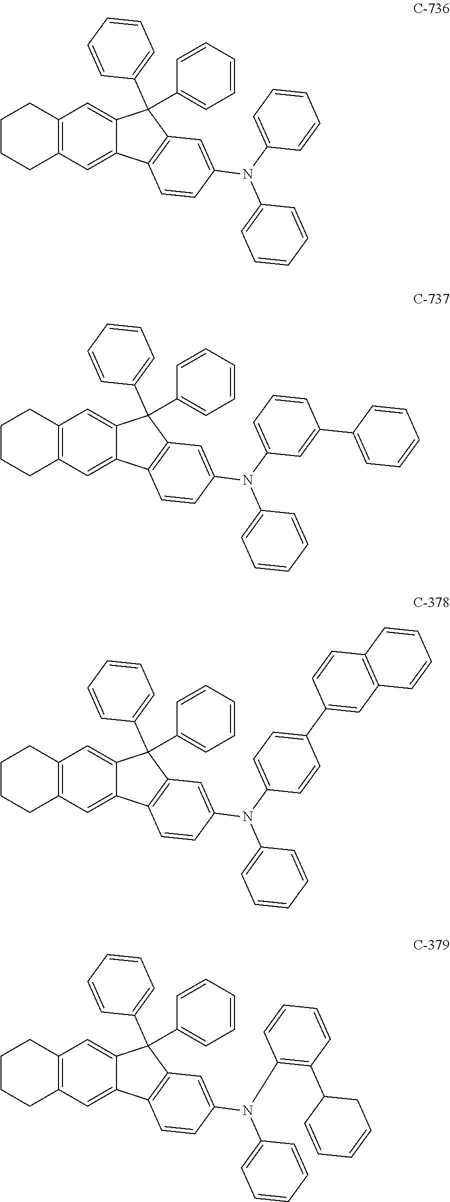 [SMILES: c1ccc(N(c2ccccc2)c2ccc3c(c2)C(c2ccccc2)(c2ccccc2)c2cc4c(cc2-3)CCCC4)cc1 ; c1ccc(-c2cccc(N(c3ccccc3)c3ccc4c(c3)C(c3ccccc3)(c3ccccc3)c3cc5c(cc3-4)CCCC5)c2)cc1 ; c1ccc(N(c2ccc(-c3ccc4ccccc4c3)cc2)c2ccc3c(c2)C(c2ccccc2)(c2ccccc2)c2cc4c(cc2-3)CCCC4)cc1 ; C1=CCC(c2ccccc2N(c2ccccc2)c2ccc3c(c2)C(c2ccccc2)(c2ccccc2)c2cc4c(cc2-3)CCCC4)C=C1]

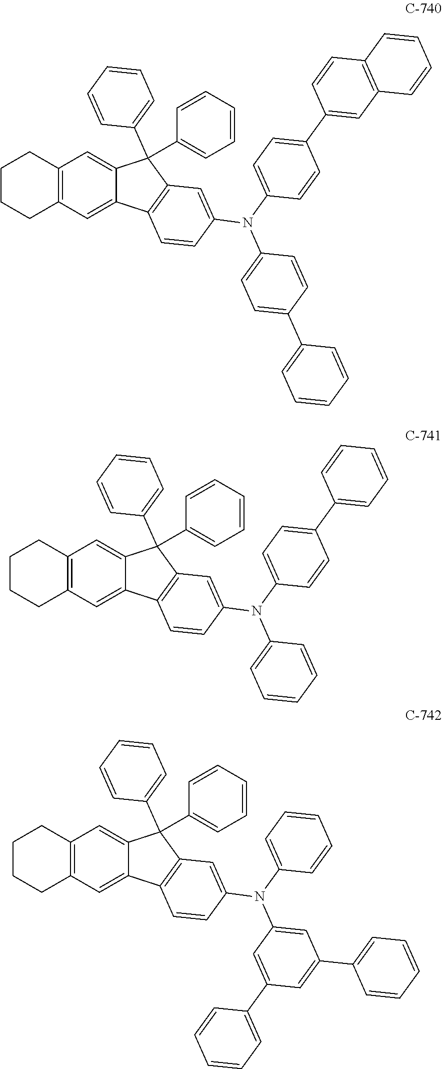

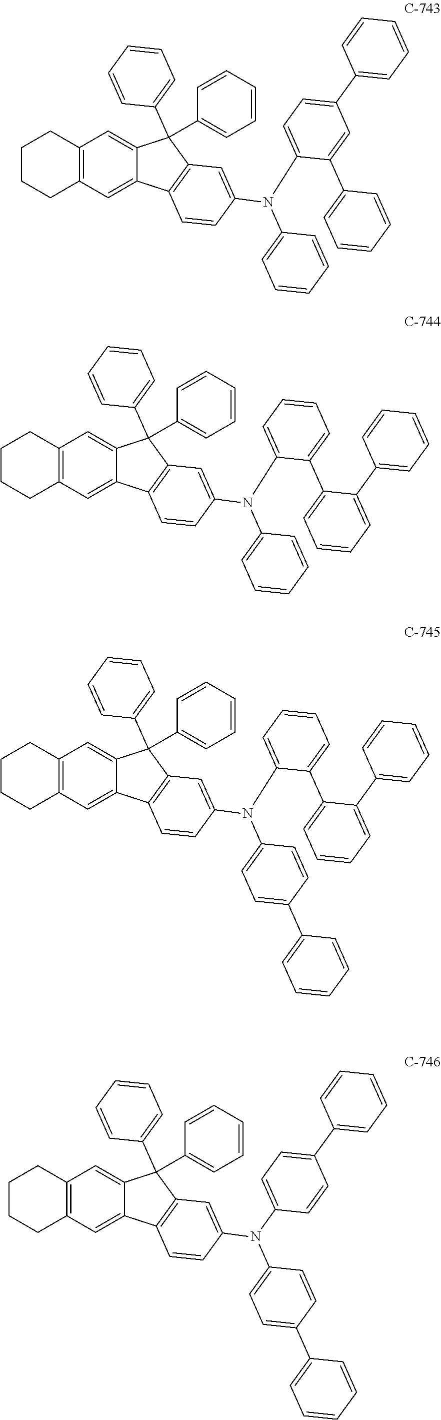 [SMILES: c1ccc(-c2ccc(N(c3ccccc3)c3ccc4c(c3)C(c3ccccc3)(c3ccccc3)c3cc5c(cc3-4)CCCC5)c(-c3ccccc3)c2)cc1 ; c1ccc(-c2ccccc2-c2ccccc2N(c2ccccc2)c2ccc3c(c2)C(c2ccccc2)(c2ccccc2)c2cc4c(cc2-3)CCCC4)cc1 ; c1ccc(-c2ccc(N(c3ccc4c(c3)C(c3ccccc3)(c3ccccc3)c3cc5c(cc3-4)CCCC5)c3ccccc3-c3ccccc3-c3ccccc3)cc2)cc1 ; c1ccc(-c2ccc(N(c3ccc(-c4ccccc4)cc3)c3ccc4c(c3)C(c3ccccc3)(c3ccccc3)c3cc5c(cc3-4)CCCC5)cc2)cc1]

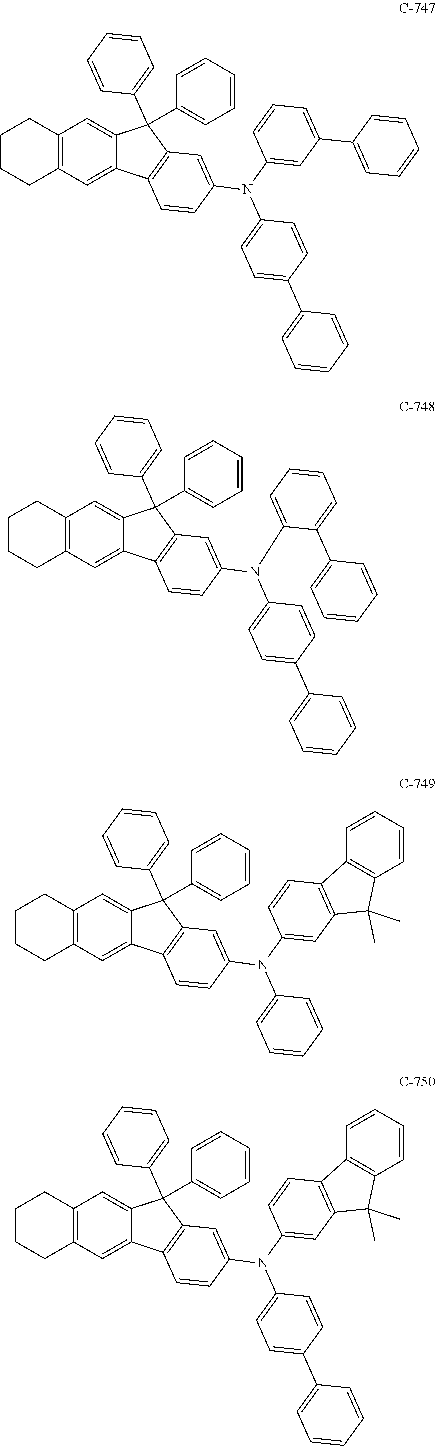 [SMILES: c1ccc(-c2ccc(N(c3cccc(-c4ccccc4)c3)c3ccc4c(c3)C(c3ccccc3)(c3ccccc3)c3cc5c(cc3-4)CCCC5)cc2)cc1 ; c1ccc(-c2ccc(N(c3ccc4c(c3)C(c3ccccc3)(c3ccccc3)c3cc5c(cc3-4)CCCC5)c3ccccc3-c3ccccc3)cc2)cc1 ; CC1(C)c2ccccc2-c2ccc(N(c3ccccc3)c3ccc4c(c3)C(c3ccccc3)(c3ccccc3)c3cc5c(cc3-4)CCCC5)cc21 ; CC1(C)c2ccccc2-c2ccc(N(c3ccc(-c4ccccc4)cc3)c3ccc4c(c3)C(c3ccccc3)(c3ccccc3)c3cc5c(cc3-4)CCCC5)cc21]

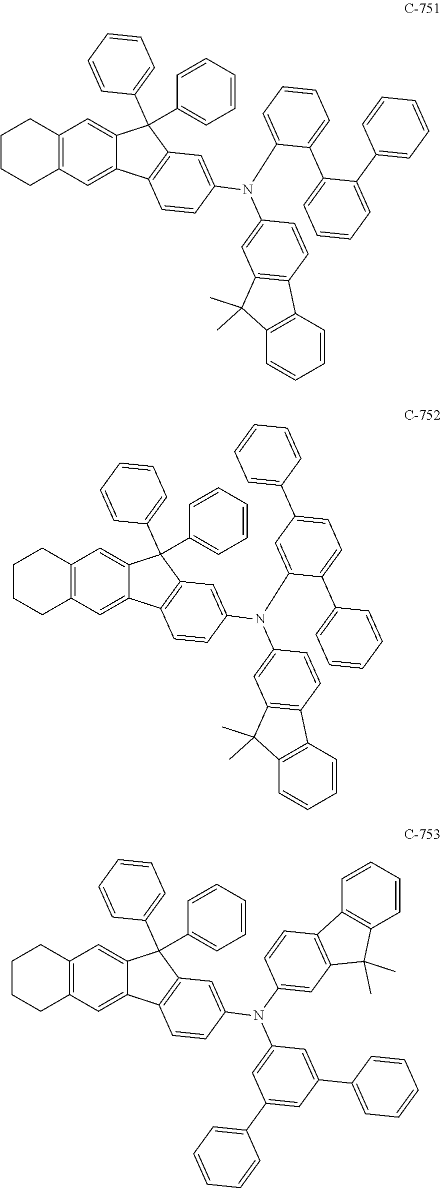 [SMILES: CC1(C)c2ccccc2-c2ccc(N(c3ccc4c(c3)C(c3ccccc3)(c3ccccc3)c3cc5c(cc3-4)CCCC5)c3ccccc3-c3ccccc3-c3ccccc3)cc21 ; CC1(C)c2ccccc2-c2ccc(N(c3ccc4c(c3)C(c3ccccc3)(c3ccccc3)c3cc5c(cc3-4)CCCC5)c3cc(-c4ccccc4)ccc3-c3ccccc3)cc21 ; CC1(C)c2ccccc2-c2ccc(N(c3cc(-c4ccccc4)cc(-c4ccccc4)c3)c3ccc4c(c3)C(c3ccccc3)(c3ccccc3)c3cc5c(cc3-4)CCCC5)cc21]

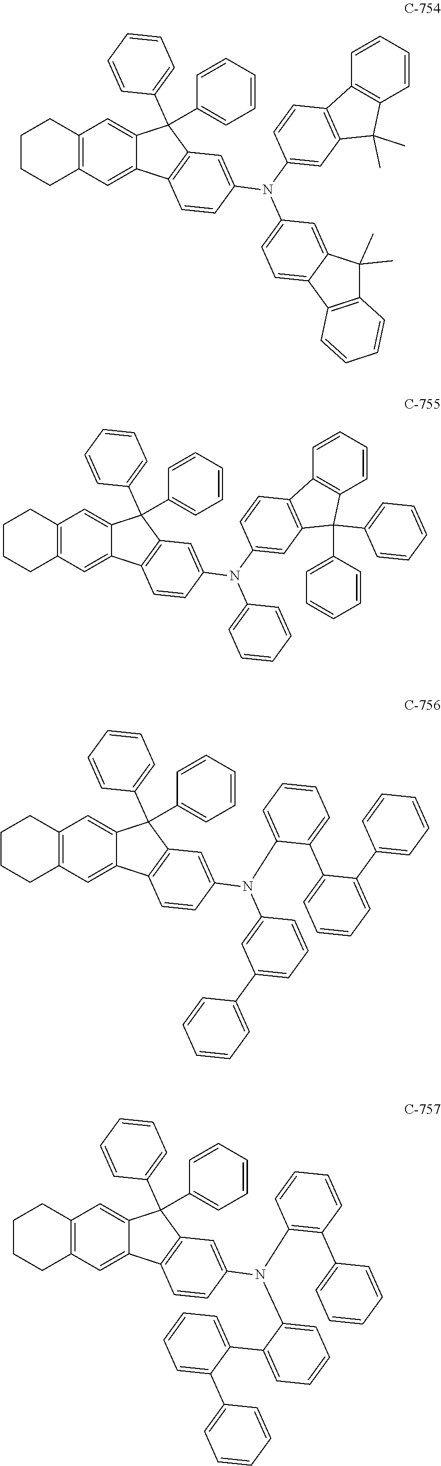 [SMILES: CC1(C)c2ccccc2-c2ccc(N(c3ccc4c(c3)C(C)(C)c3ccccc3-4)c3ccc4c(c3)C(c3ccccc3)(c3ccccc3)c3cc5c(cc3-4)CCCC5)cc21 ; c1ccc(N(c2ccc3c(c2)C(c2ccccc2)(c2ccccc2)c2ccccc2-3)c2ccc3c(c2)C(c2ccccc2)(c2ccccc2)c2cc4c(cc2-3)CCCC4)cc1 ; c1ccc(-c2cccc(N(c3ccc4c(c3)C(c3ccccc3)(c3ccccc3)c3cc5c(cc3-4)CCCC5)c3ccccc3-c3ccccc3-c3ccccc3)c2)cc1 ; c1ccc(-c2ccccc2-c2ccccc2N(c2ccc3c(c2)C(c2ccccc2)(c2ccccc2)c2cc4c(cc2-3)CCCC4)c2ccccc2-c2ccccc2)cc1]

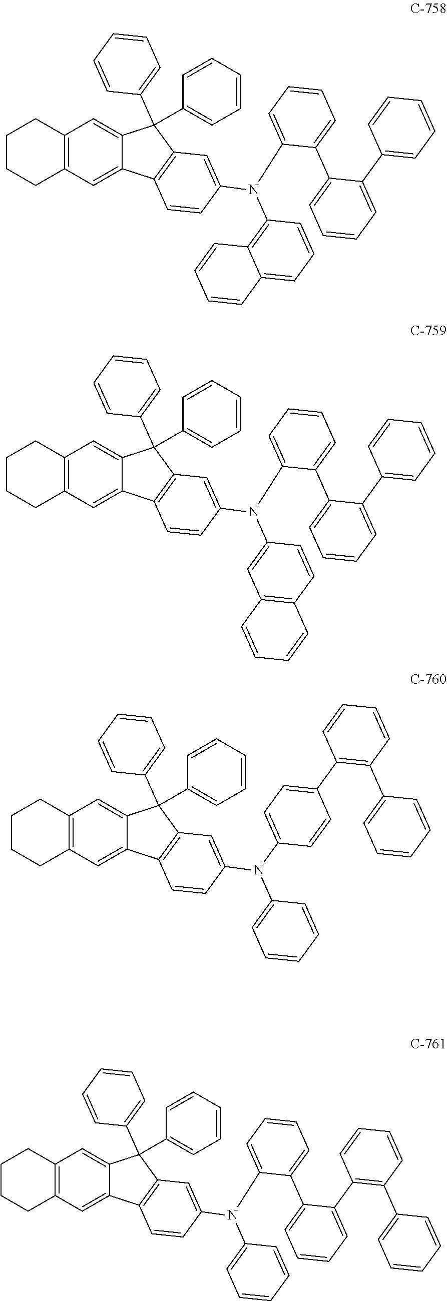 [SMILES: c1ccc(-c2ccccc2-c2ccccc2N(c2ccc3c(c2)C(c2ccccc2)(c2ccccc2)c2cc4c(cc2-3)CCCC4)c2cccc3ccccc23)cc1 ; c1ccc(-c2ccccc2-c2ccccc2N(c2ccc3c(c2)C(c2ccccc2)(c2ccccc2)c2cc4c(cc2-3)CCCC4)c2ccc3ccccc3c2)cc1 ; c1ccc(-c2ccccc2-c2ccc(N(c3ccccc3)c3ccc4c(c3)C(c3ccccc3)(c3ccccc3)c3cc5c(cc3-4)CCCC5)cc2)cc1 ; c1ccc(-c2ccccc2-c2ccccc2-c2ccccc2N(c2ccccc2)c2ccc3c(c2)C(c2ccccc2)(c2ccccc2)c2cc4c(cc2-3)CCCC4)cc1]

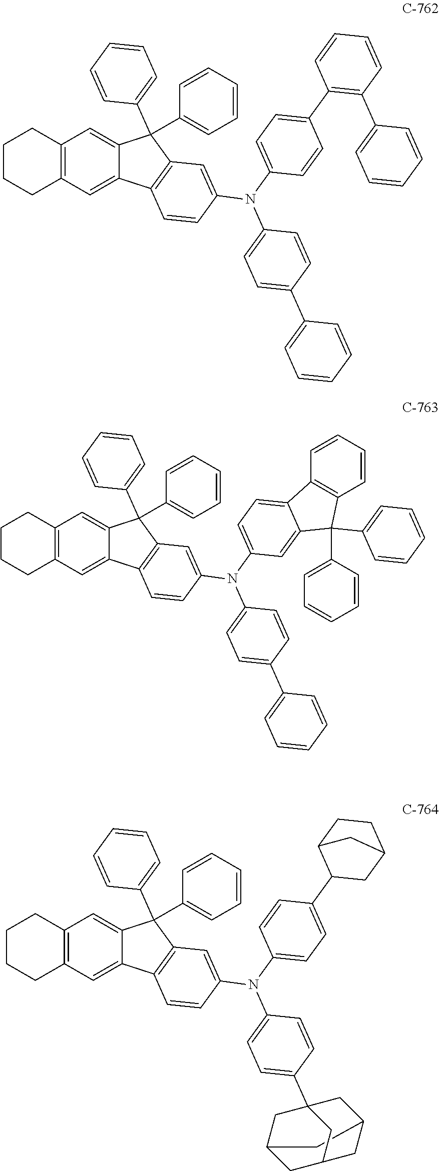 [SMILES: c1ccc(-c2ccc(N(c3ccc(-c4ccccc4-c4ccccc4)cc3)c3ccc4c(c3)C(c3ccccc3)(c3ccccc3)c3cc5c(cc3-4)CCCC5)cc2)cc1 ; c1ccc(-c2ccc(N(c3ccc4c(c3)C(c3ccccc3)(c3ccccc3)c3ccccc3-4)c3ccc4c(c3)C(c3ccccc3)(c3ccccc3)c3cc5c(cc3-4)CCCC5)cc2)cc1 ; c1ccc(C2(c3ccccc3)c3cc(N(c4ccc(C5CC6CCC5C6)cc4)c4ccc(C56CC7CC(CC(C7)C5)C6)cc4)ccc3-c3cc4c(cc32)CCCC4)cc1]

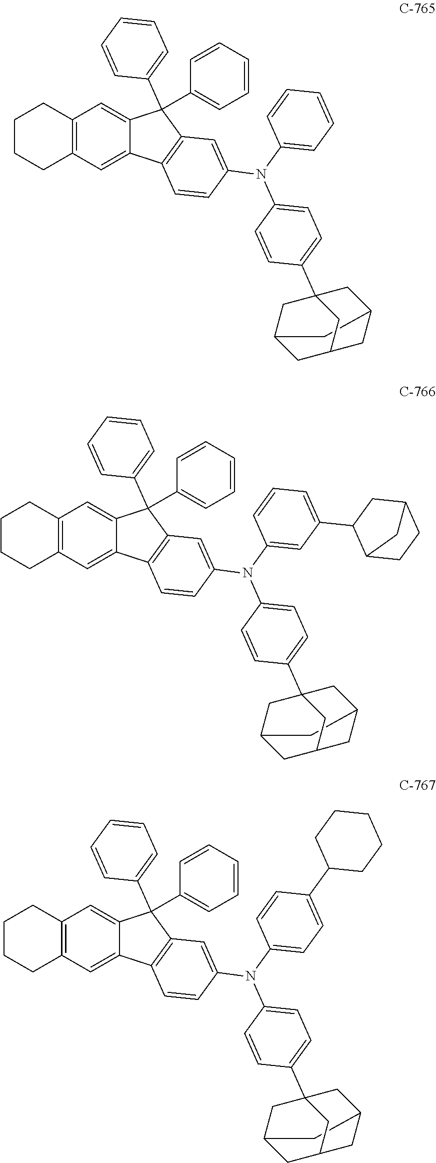 [SMILES: c1ccc(N(c2ccc(C34CC5CC(CC(C5)C3)C4)cc2)c2ccc3c(c2)C(c2ccccc2)(c2ccccc2)c2cc4c(cc2-3)CCCC4)cc1 ; c1ccc(C2(c3ccccc3)c3cc(N(c4ccc(C56CC7CC(CC(C7)C5)C6)cc4)c4cccc(C5CC6CCC5C6)c4)ccc3-c3cc4c(cc32)CCCC4)cc1 ; c1ccc(C2(c3ccccc3)c3cc(N(c4ccc(C5CCCCC5)cc4)c4ccc(C56CC7CC(CC(C7)C5)C6)cc4)ccc3-c3cc4c(cc32)CCCC4)cc1]

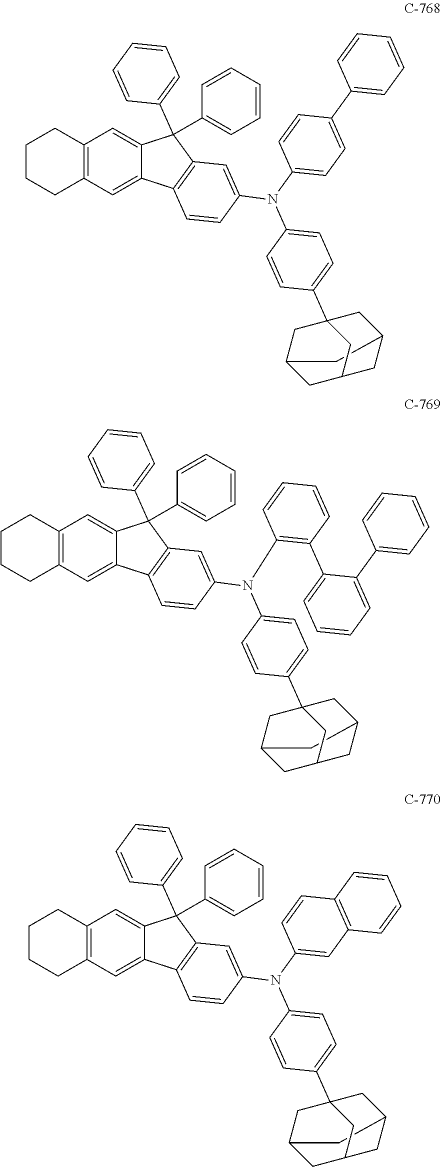 [SMILES: c1ccc(-c2ccc(N(c3ccc(C45CC6CC(CC(C6)C4)C5)cc3)c3ccc4c(c3)C(c3ccccc3)(c3ccccc3)c3cc5c(cc3-4)CCCC5)cc2)cc1 ; c1ccc(-c2ccccc2-c2ccccc2N(c2ccc(C34CC5CC(CC(C5)C3)C4)cc2)c2ccc3c(c2)C(c2ccccc2)(c2ccccc2)c2cc4c(cc2-3)CCCC4)cc1 ; c1ccc(C2(c3ccccc3)c3cc(N(c4ccc(C56CC7CC(CC(C7)C5)C6)cc4)c4ccc5ccccc5c4)ccc3-c3cc4c(cc32)CCCC4)cc1]

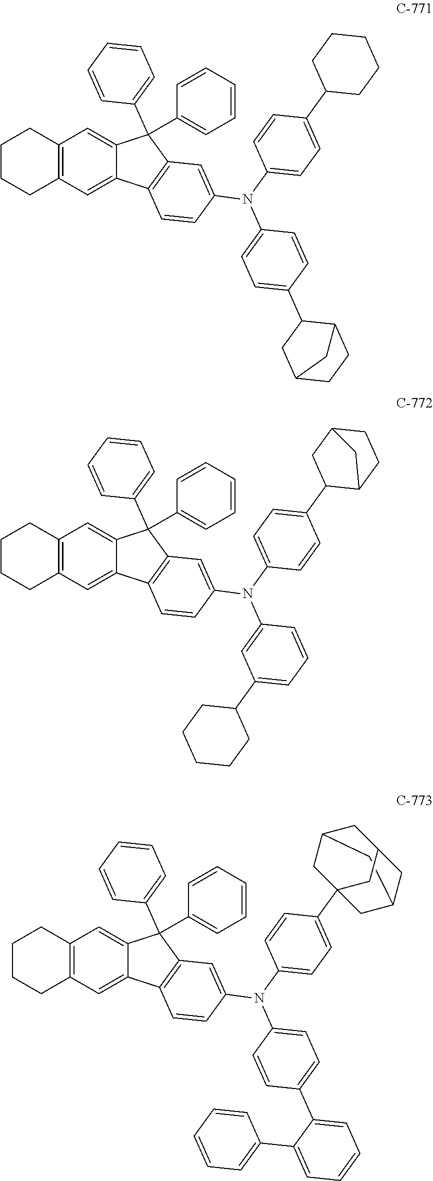 [SMILES: c1ccc(C2(c3ccccc3)c3cc(N(c4ccc(C5CCCCC5)cc4)c4ccc(C5CC6CCC5C6)cc4)ccc3-c3cc4c(cc32)CCCC4)cc1 ; c1ccc(C2(c3ccccc3)c3cc(N(c4ccc(C5CC6CCC5C6)cc4)c4cccc(C5CCCCC5)c4)ccc3-c3cc4c(cc32)CCCC4)cc1 ; c1ccc(-c2ccccc2-c2ccc(N(c3ccc(C45CC6CC(CC(C6)C4)C5)cc3)c3ccc4c(c3)C(c3ccccc3)(c3ccccc3)c3cc5c(cc3-4)CCCC5)cc2)cc1]

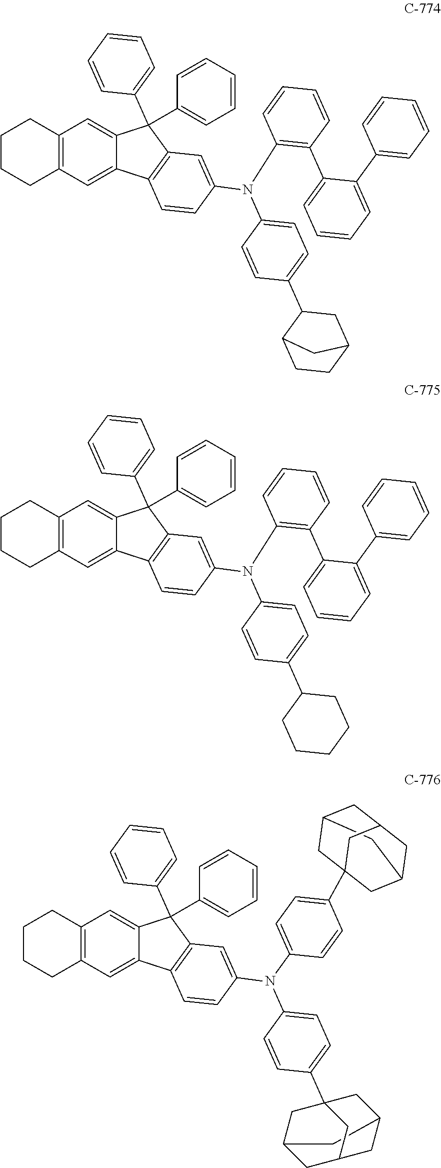 [SMILES: c1ccc(-c2ccccc2-c2ccccc2N(c2ccc(C3CC4CCC3C4)cc2)c2ccc3c(c2)C(c2ccccc2)(c2ccccc2)c2cc4c(cc2-3)CCCC4)cc1 ; c1ccc(-c2ccccc2-c2ccccc2N(c2ccc(C3CCCCC3)cc2)c2ccc3c(c2)C(c2ccccc2)(c2ccccc2)c2cc4c(cc2-3)CCCC4)cc1 ; c1ccc(C2(c3ccccc3)c3cc(N(c4ccc(C56CC7CC(CC(C7)C5)C6)cc4)c4ccc(C56CC7CC(CC(C7)C5)C6)cc4)ccc3-c3cc4c(cc32)CCCC4)cc1]

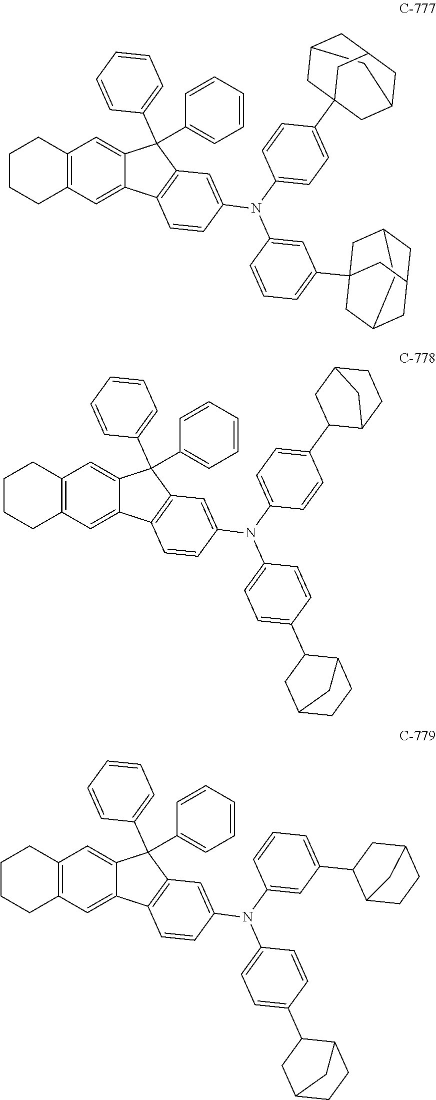

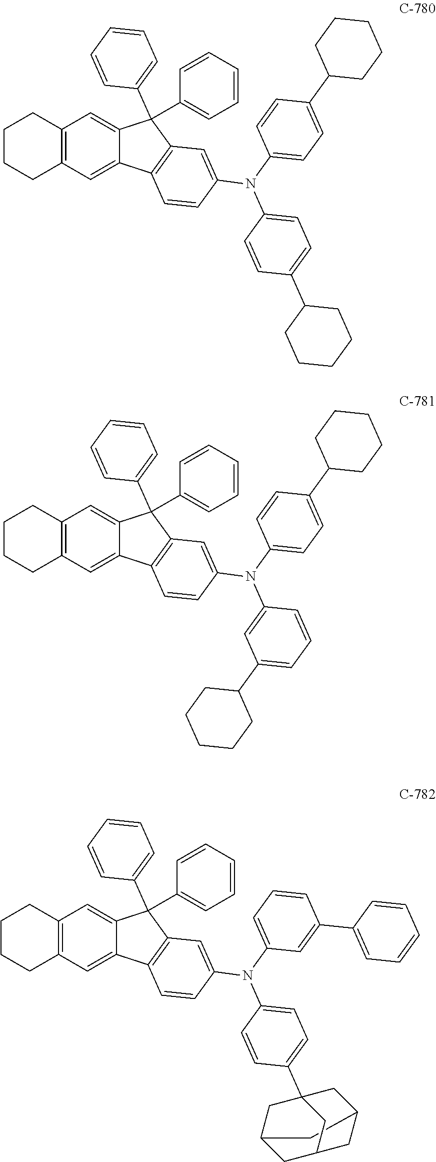

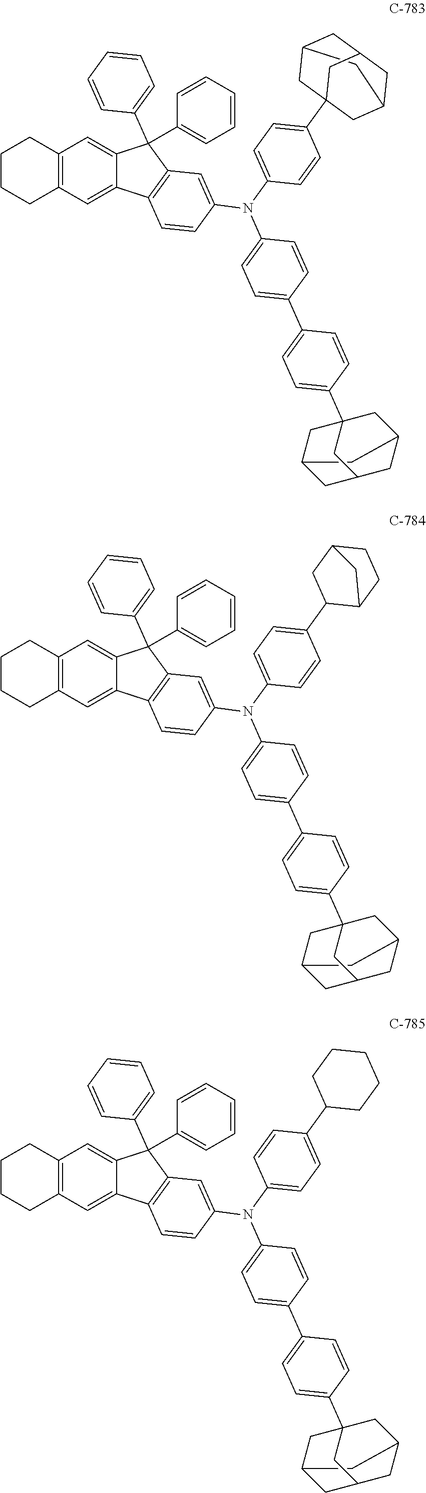 [SMILES: c1ccc(C2(c3ccccc3)c3cc(N(c4ccc(-c5ccc(C67CC8CC(CC(C8)C6)C7)cc5)cc4)c4ccc(C56CC7CC(CC(C7)C5)C6)cc4)ccc3-c3cc4c(cc32)CCCC4)cc1 ; c1ccc(C2(c3ccccc3)c3cc(N(c4ccc(-c5ccc(C67CC8CC(CC(C8)C6)C7)cc5)cc4)c4ccc(C5CC6CCC5C6)cc4)ccc3-c3cc4c(cc32)CCCC4)cc1 ; c1ccc(C2(c3ccccc3)c3cc(N(c4ccc(-c5ccc(C67CC8CC(CC(C8)C6)C7)cc5)cc4)c4ccc(C5CCCCC5)cc4)ccc3-c3cc4c(cc32)CCCC4)cc1]

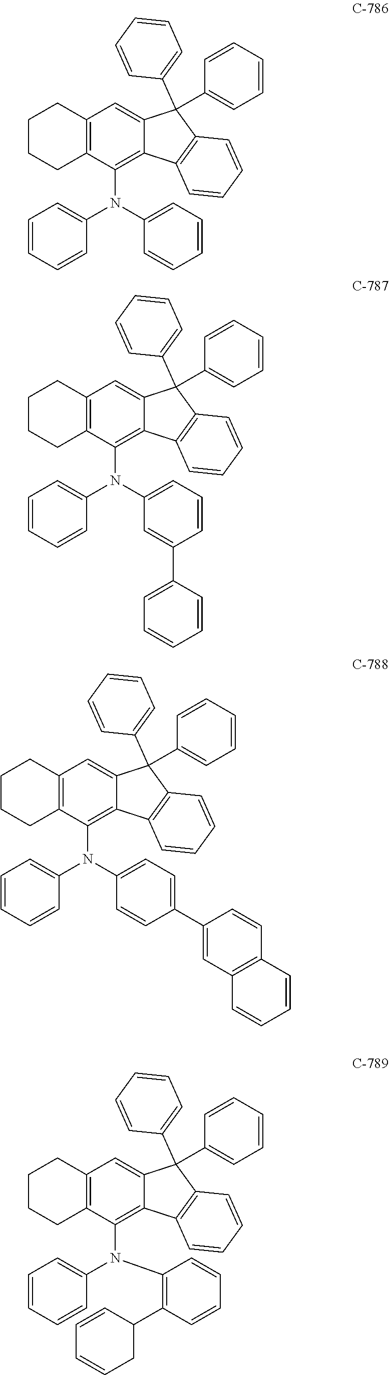

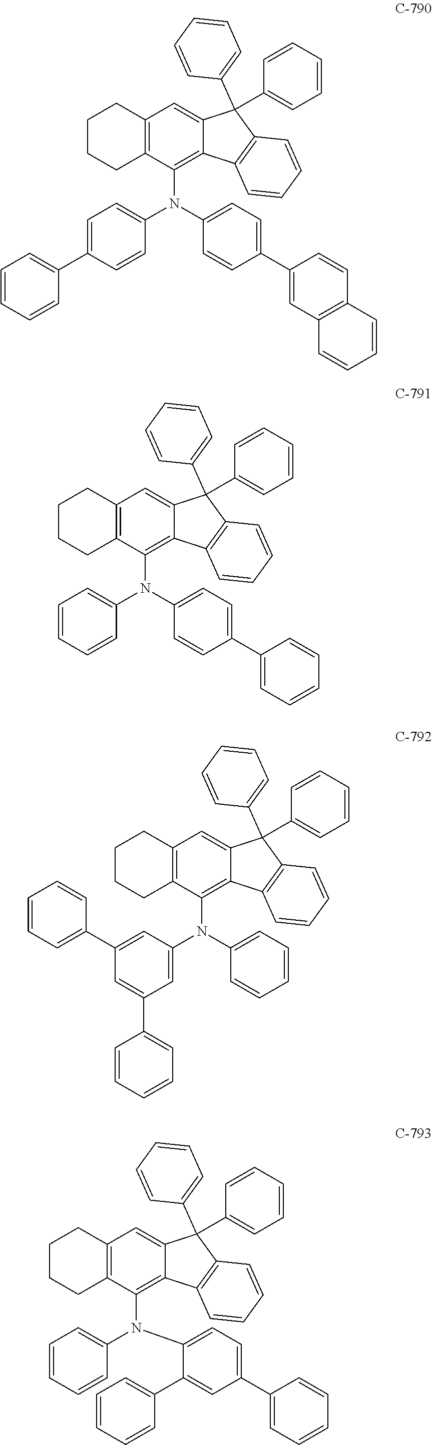

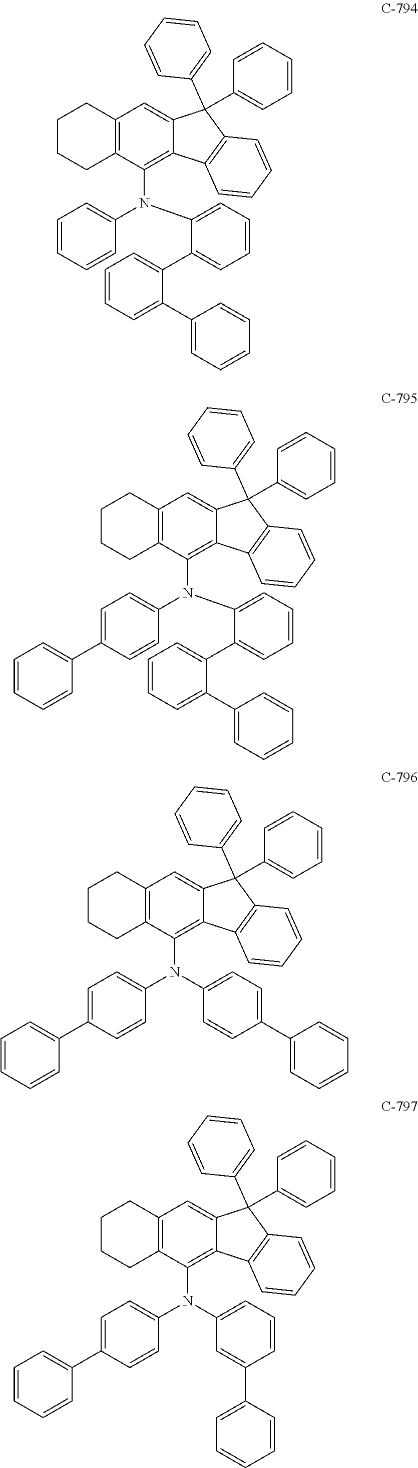

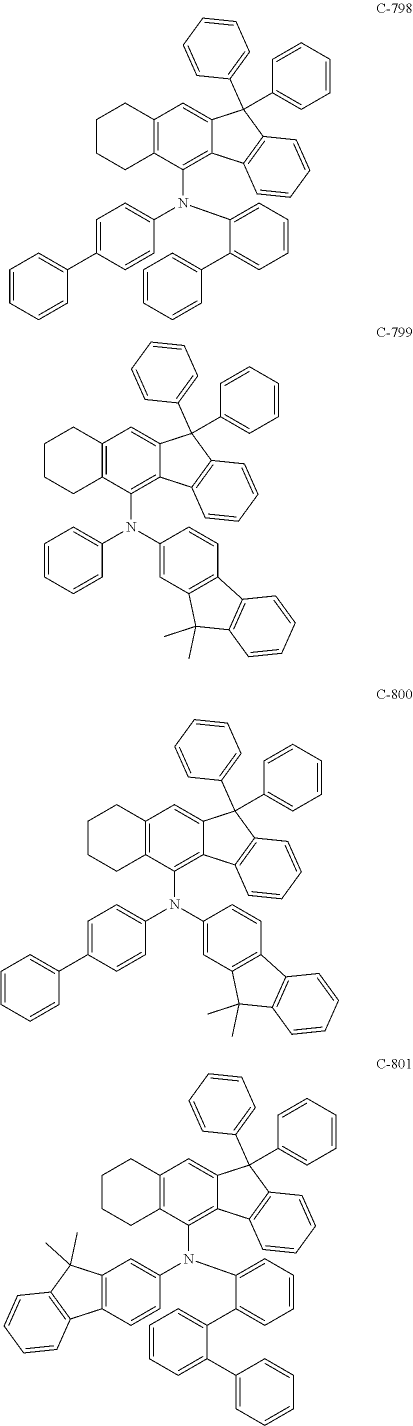

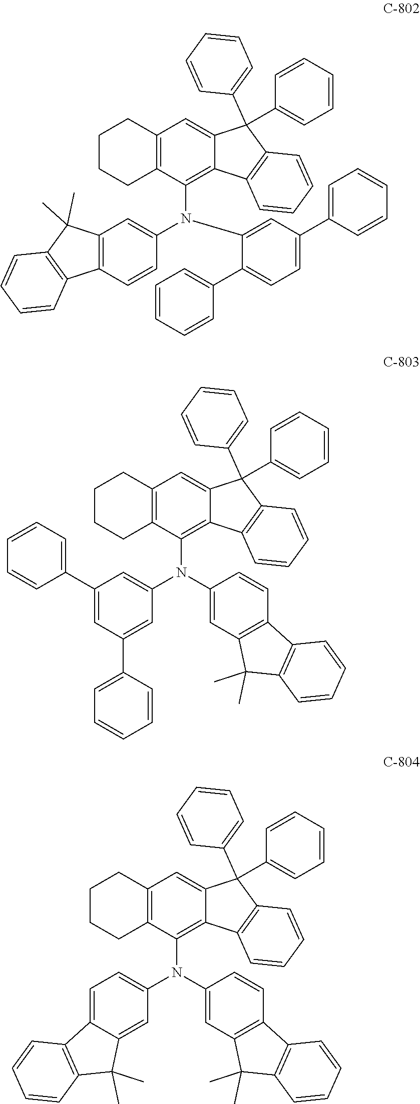 [SMILES: CC1(C)c2ccccc2-c2ccc(N(c3cc(-c4ccccc4)ccc3-c3ccccc3)c3c4c(cc5c3-c3ccccc3C5(c3ccccc3)c3ccccc3)CCCC4)cc21 ; CC1(C)c2ccccc2-c2ccc(N(c3cc(-c4ccccc4)cc(-c4ccccc4)c3)c3c4c(cc5c3-c3ccccc3C5(c3ccccc3)c3ccccc3)CCCC4)cc21 ; CC1(C)c2ccccc2-c2ccc(N(c3ccc4c(c3)C(C)(C)c3ccccc3-4)c3c4c(cc5c3-c3ccccc3C5(c3ccccc3)c3ccccc3)CCCC4)cc21]

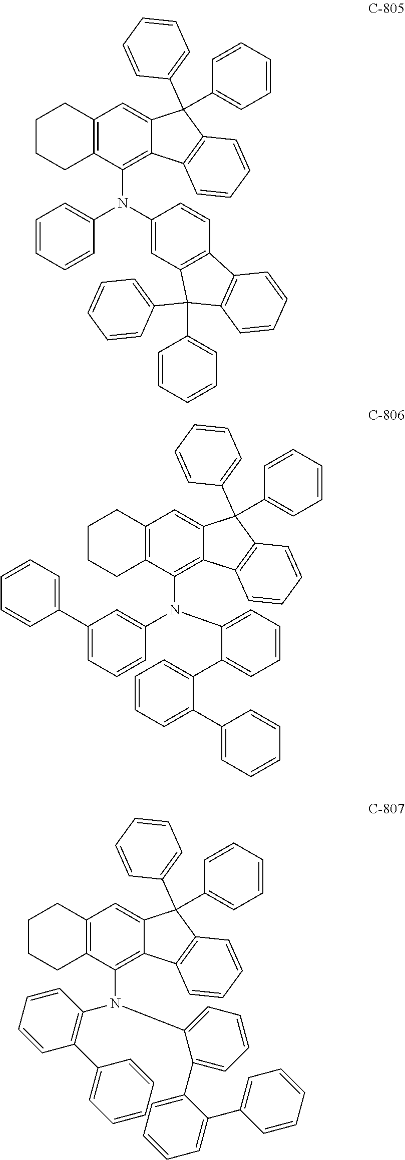

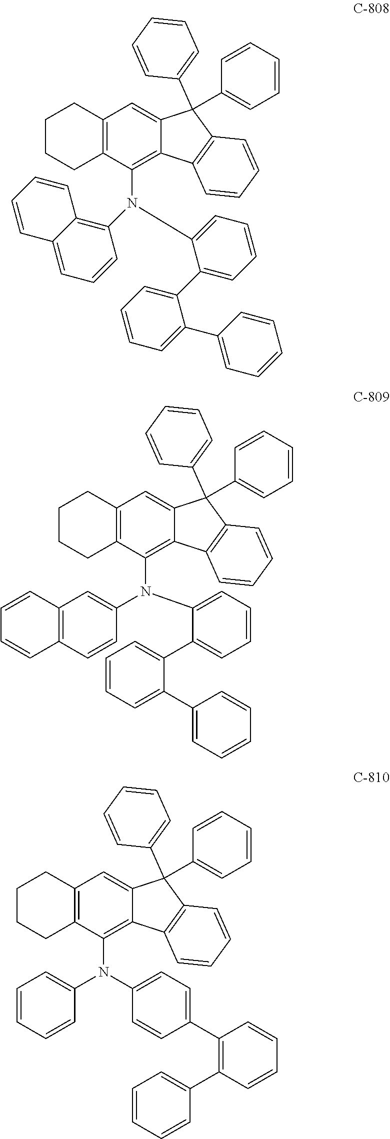 [SMILES: c1ccc(-c2ccccc2-c2ccccc2N(c2c3c(cc4c2-c2ccccc2C4(c2ccccc2)c2ccccc2)CCCC3)c2cccc3ccccc23)cc1 ; c1ccc(-c2ccccc2-c2ccccc2N(c2ccc3ccccc3c2)c2c3c(cc4c2-c2ccccc2C4(c2ccccc2)c2ccccc2)CCCC3)cc1 ; c1ccc(-c2ccccc2-c2ccc(N(c3ccccc3)c3c4c(cc5c3-c3ccccc3C5(c3ccccc3)c3ccccc3)CCCC4)cc2)cc1]

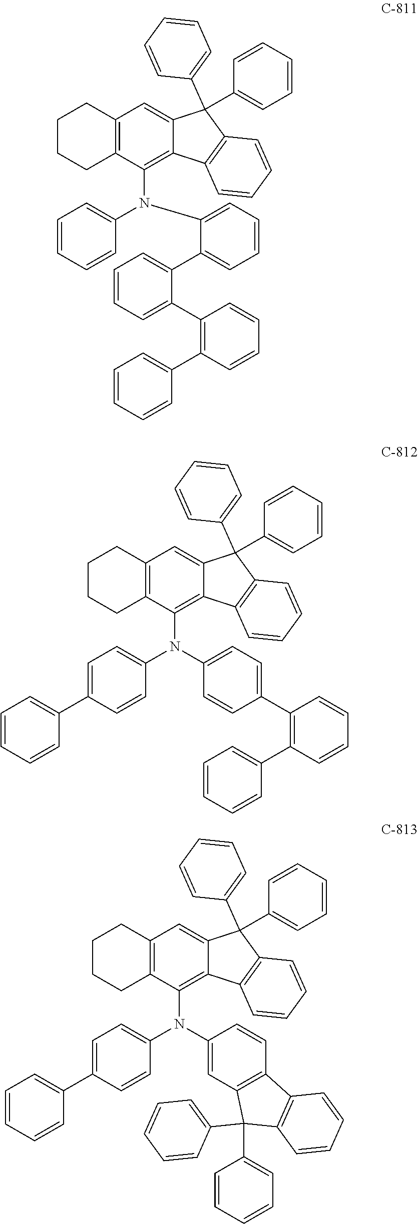

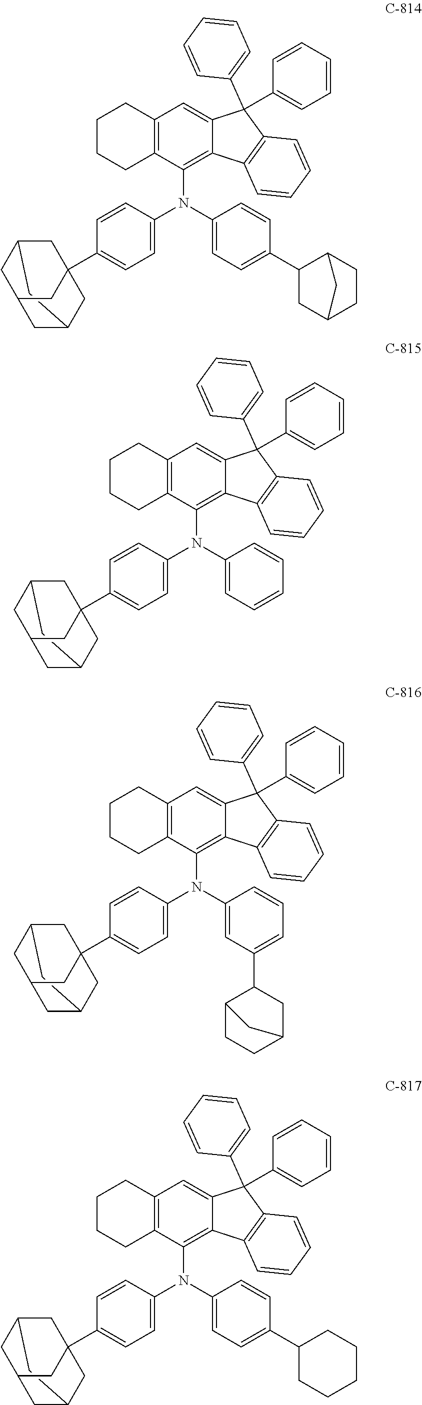 [SMILES: c1ccc(C2(c3ccccc3)c3ccccc3-c3c2cc2c(c3N(c3ccc(C4CC5CCC4C5)cc3)c3ccc(C45CC6CC(CC(C6)C4)C5)cc3)CCCC2)cc1 ; c1ccc(N(c2ccc(C34CC5CC(CC(C5)C3)C4)cc2)c2c3c(cc4c2-c2ccccc2C4(c2ccccc2)c2ccccc2)CCCC3)cc1 ; c1ccc(C2(c3ccccc3)c3ccccc3-c3c2cc2c(c3N(c3ccc(C45CC6CC(CC(C6)C4)C5)cc3)c3cccc(C4CC5CCC4C5)c3)CCCC2)cc1 ; c1ccc(C2(c3ccccc3)c3ccccc3-c3c2cc2c(c3N(c3ccc(C4CCCCC4)cc3)c3ccc(C45CC6CC(CC(C6)C4)C5)cc3)CCCC2)cc1]

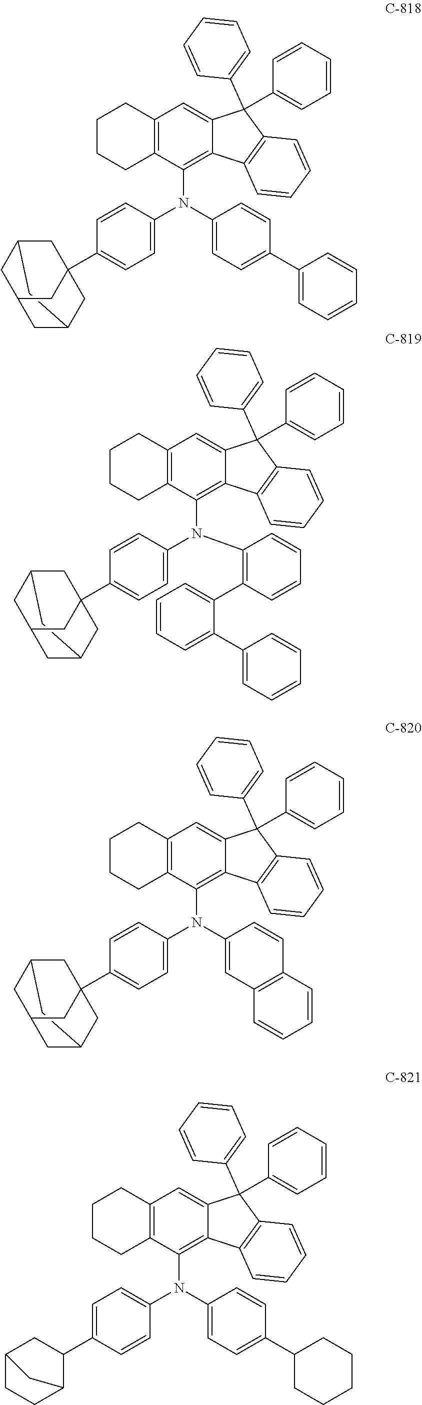

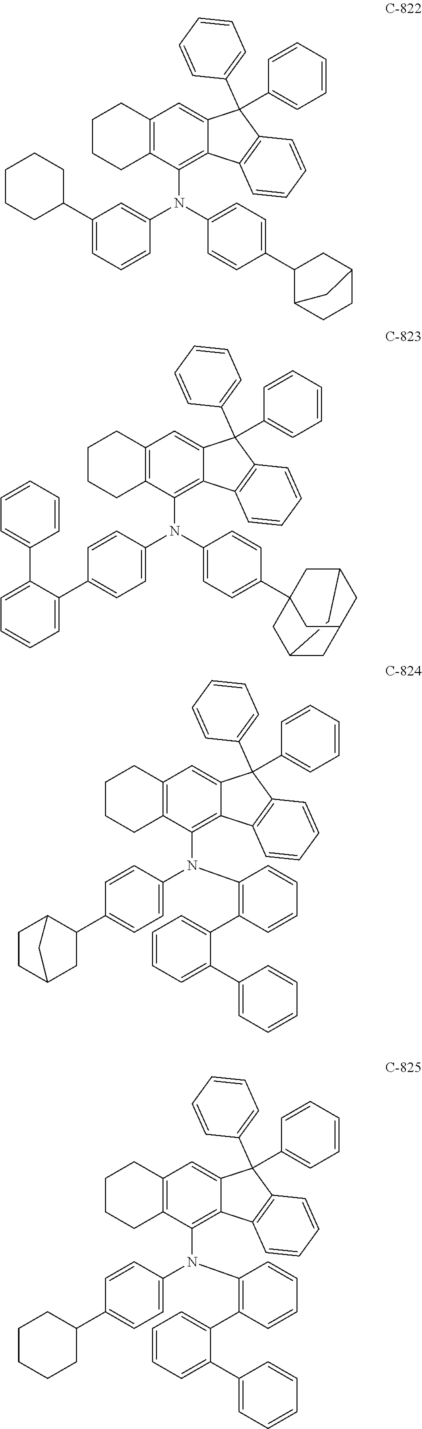 [SMILES: c1ccc(C2(c3ccccc3)c3ccccc3-c3c2cc2c(c3N(c3ccc(C4CC5CCC4C5)cc3)c3cccc(C4CCCCC4)c3)CCCC2)cc1 ; c1ccc(-c2ccccc2-c2ccc(N(c3ccc(C45CC6CC(CC(C6)C4)C5)cc3)c3c4c(cc5c3-c3ccccc3C5(c3ccccc3)c3ccccc3)CCCC4)cc2)cc1 ; c1ccc(-c2ccccc2-c2ccccc2N(c2ccc(C3CC4CCC3C4)cc2)c2c3c(cc4c2-c2ccccc2C4(c2ccccc2)c2ccccc2)CCCC3)cc1 ; c1ccc(-c2ccccc2-c2ccccc2N(c2ccc(C3CCCCC3)cc2)c2c3c(cc4c2-c2ccccc2C4(c2ccccc2)c2ccccc2)CCCC3)cc1]

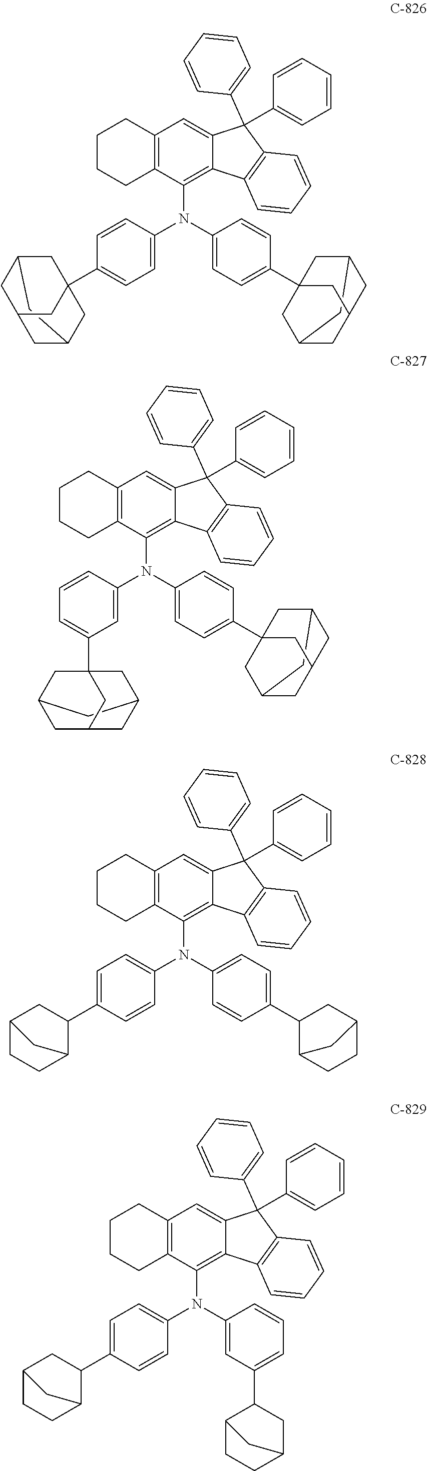 [SMILES: c1ccc(C2(c3ccccc3)c3ccccc3-c3c2cc2c(c3N(c3ccc(C45CC6CC(CC(C6)C4)C5)cc3)c3ccc(C45CC6CC(CC(C6)C4)C5)cc3)CCCC2)cc1 ; c1ccc(C2(c3ccccc3)c3ccccc3-c3c2cc2c(c3N(c3ccc(C45CC6CC(CC(C6)C4)C5)cc3)c3cccc(C45CC6CC(CC(C6)C4)C5)c3)CCCC2)cc1 ; c1ccc(C2(c3ccccc3)c3ccccc3-c3c2cc2c(c3N(c3ccc(C4CC5CCC4C5)cc3)c3ccc(C4CC5CCC4C5)cc3)CCCC2)cc1 ; c1ccc(C2(c3ccccc3)c3ccccc3-c3c2cc2c(c3N(c3ccc(C4CC5CCC4C5)cc3)c3cccc(C4CC5CCC4C5)c3)CCCC2)cc1]

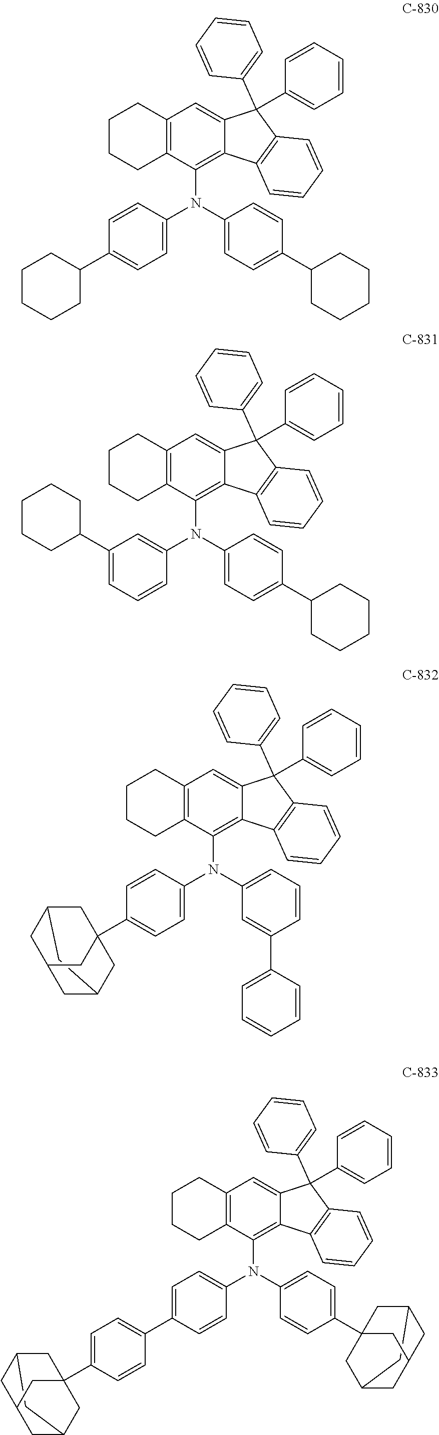 [SMILES: c1ccc(C2(c3ccccc3)c3ccccc3-c3c2cc2c(c3N(c3ccc(C4CCCCC4)cc3)c3ccc(C4CCCCC4)cc3)CCCC2)cc1 ; c1ccc(C2(c3ccccc3)c3ccccc3-c3c2cc2c(c3N(c3ccc(C4CCCCC4)cc3)c3cccc(C4CCCCC4)c3)CCCC2)cc1 ; c1ccc(-c2cccc(N(c3ccc(C45CC6CC(CC(C6)C4)C5)cc3)c3c4c(cc5c3-c3ccccc3C5(c3ccccc3)c3ccccc3)CCCC4)c2)cc1 ; c1ccc(C2(c3ccccc3)c3ccccc3-c3c2cc2c(c3N(c3ccc(-c4ccc(C56CC7CC(CC(C7)C5)C6)cc4)cc3)c3ccc(C45CC6CC(CC(C6)C4)C5)cc3)CCCC2)cc1]

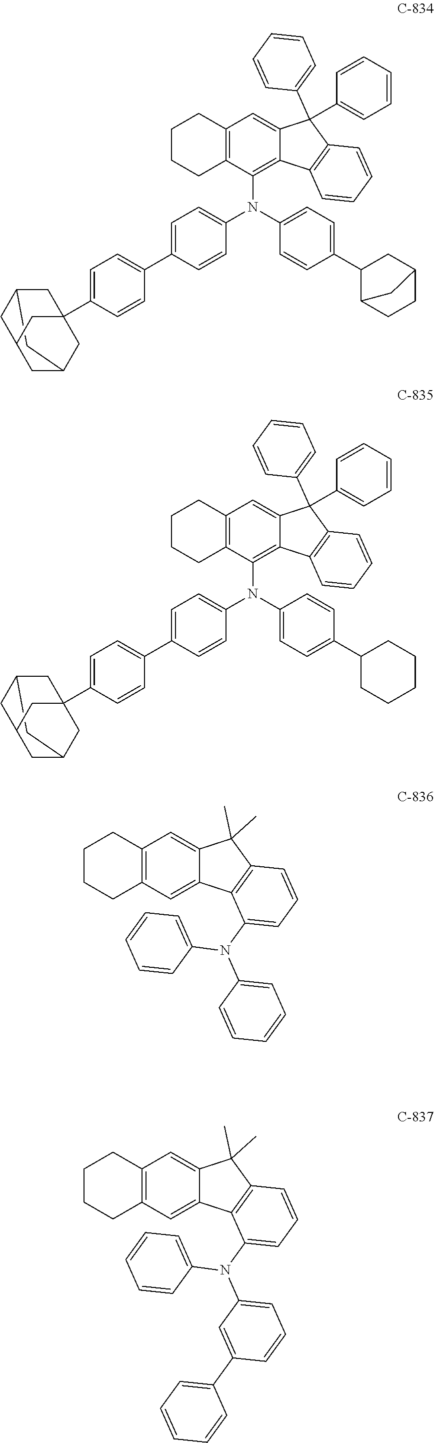 [SMILES: c1ccc(C2(c3ccccc3)c3ccccc3-c3c2cc2c(c3N(c3ccc(-c4ccc(C56CC7CC(CC(C7)C5)C6)cc4)cc3)c3ccc(C4CC5CCC4C5)cc3)CCCC2)cc1 ; c1ccc(C2(c3ccccc3)c3ccccc3-c3c2cc2c(c3N(c3ccc(-c4ccc(C56CC7CC(CC(C7)C5)C6)cc4)cc3)c3ccc(C4CCCCC4)cc3)CCCC2)cc1 ; CC1(C)c2cc3c(cc2-c2c(N(c4ccccc4)c4ccccc4)cccc21)CCCC3 ; CC1(C)c2cc3c(cc2-c2c(N(c4ccccc4)c4cccc(-c5ccccc5)c4)cccc21)CCCC3]

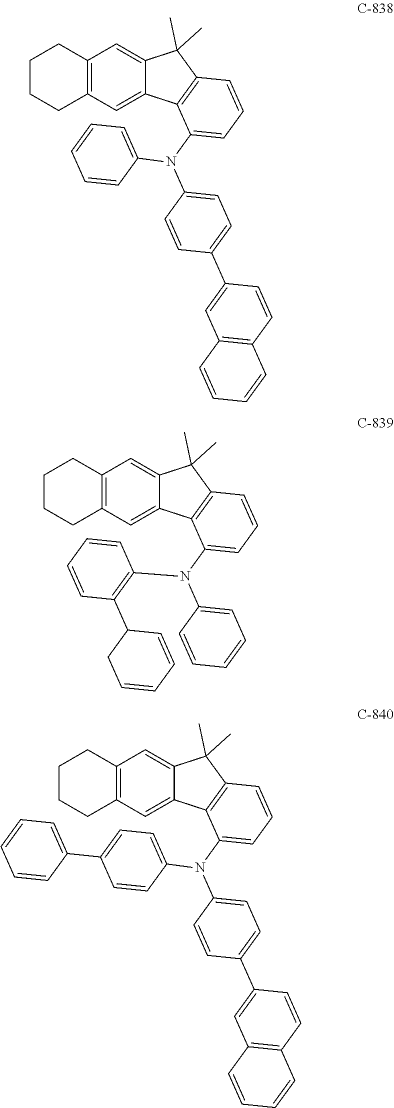

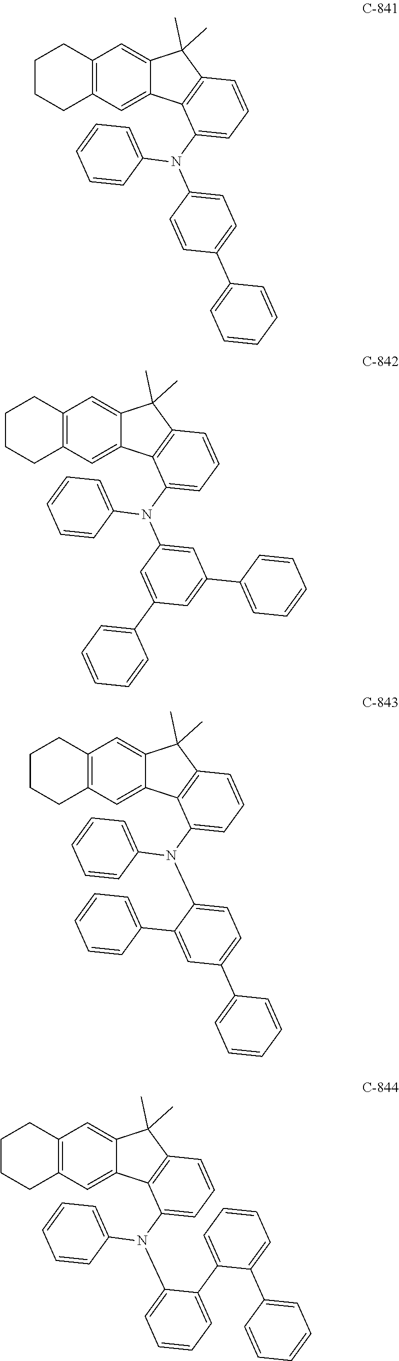 [SMILES: CC1(C)c2cc3c(cc2-c2c(N(c4ccccc4)c4ccc(-c5ccccc5)cc4)cccc21)CCCC3 ; CC1(C)c2cc3c(cc2-c2c(N(c4ccccc4)c4cc(-c5ccccc5)cc(-c5ccccc5)c4)cccc21)CCCC3 ; CC1(C)c2cc3c(cc2-c2c(N(c4ccccc4)c4ccc(-c5ccccc5)cc4-c4ccccc4)cccc21)CCCC3 ; CC1(C)c2cc3c(cc2-c2c(N(c4ccccc4)c4ccccc4-c4ccccc4-c4ccccc4)cccc21)CCCC3]

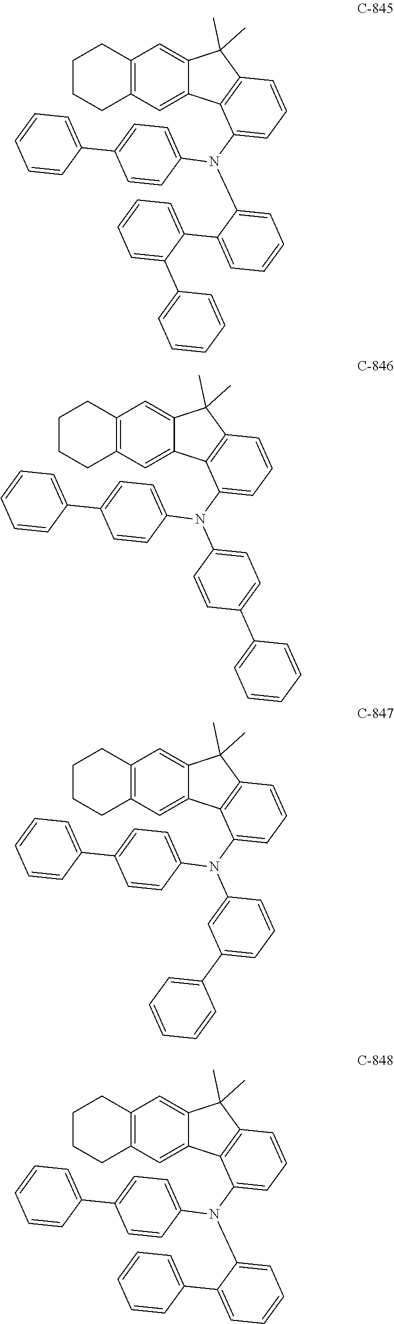

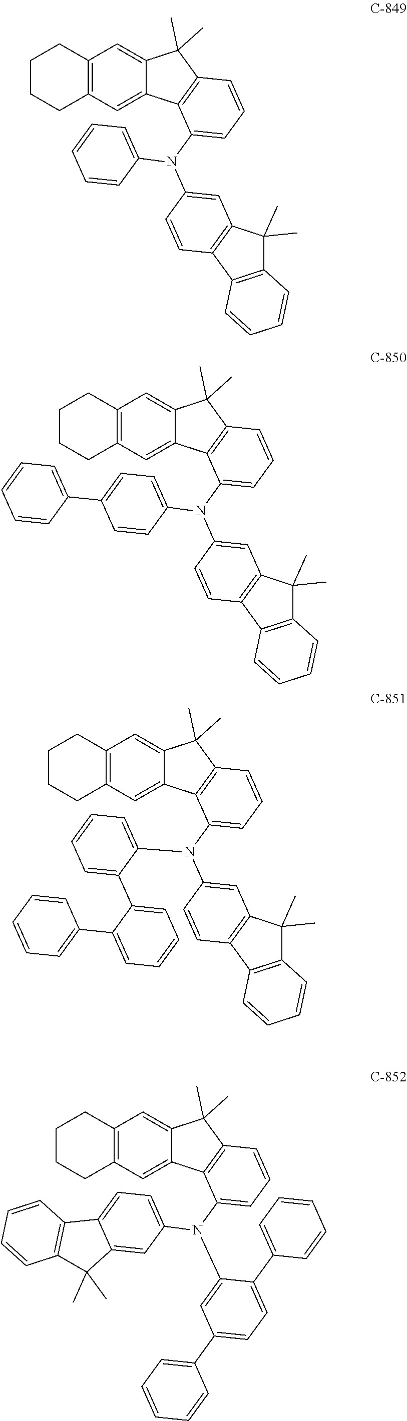

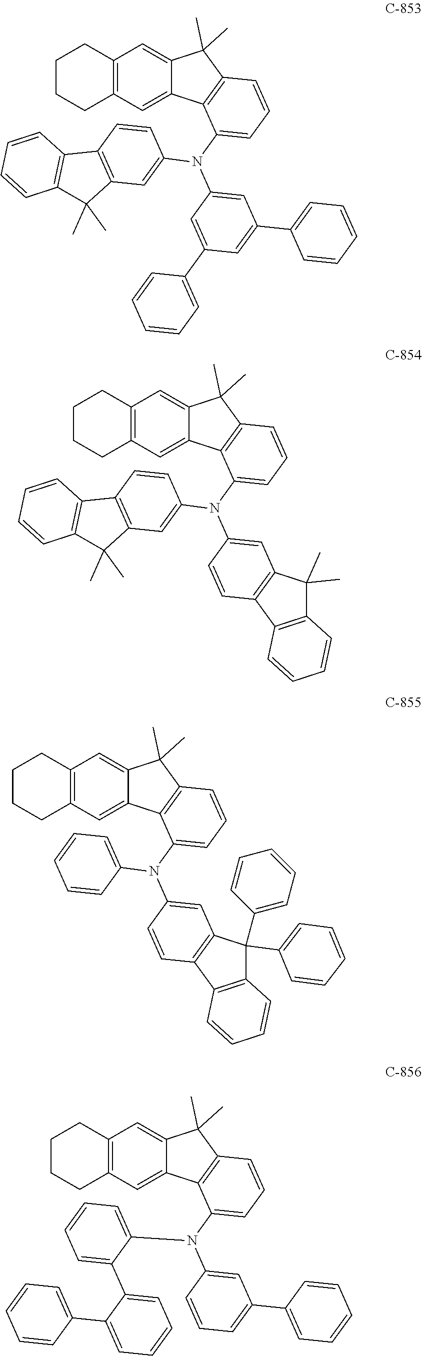

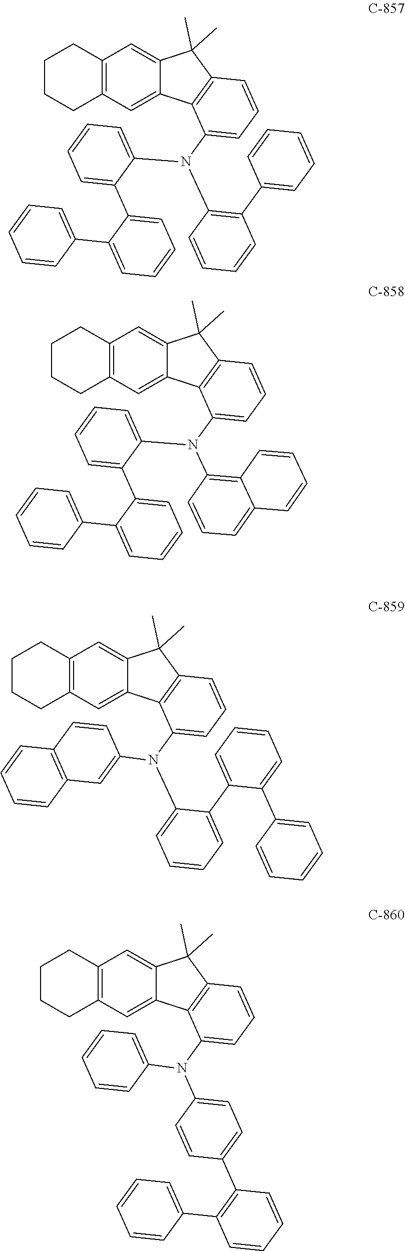

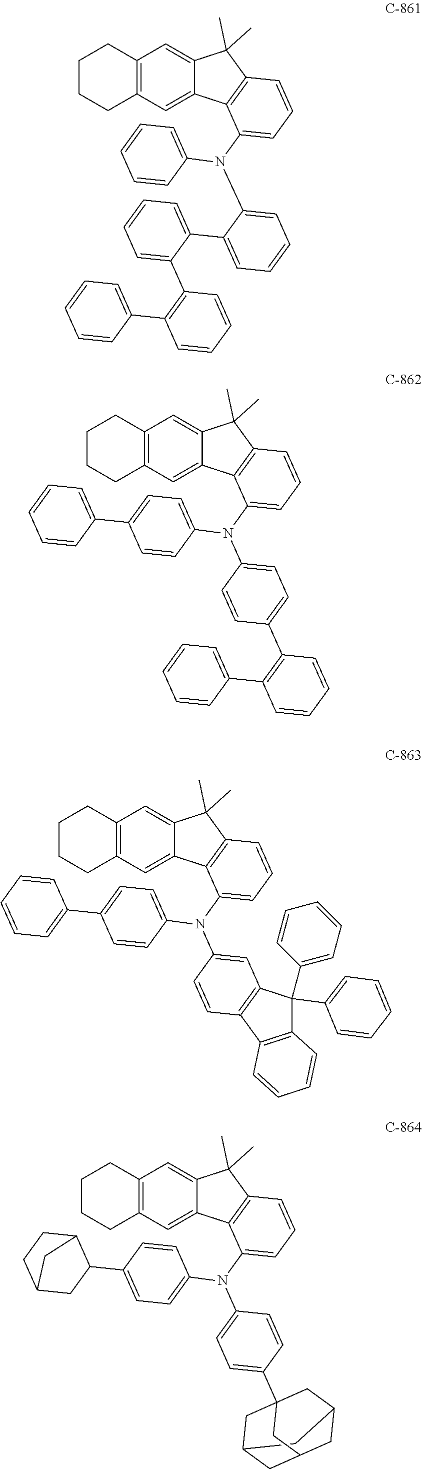

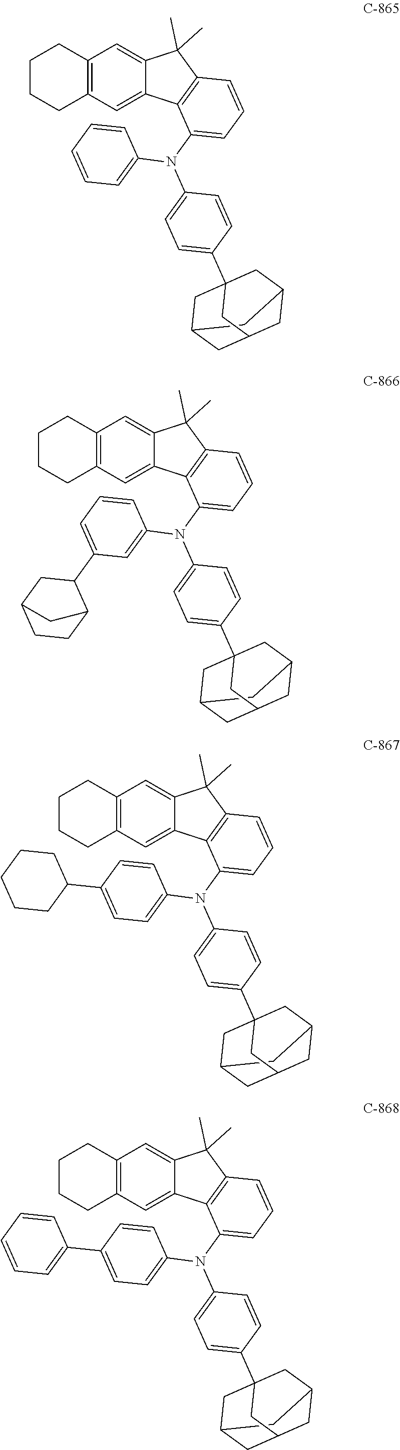

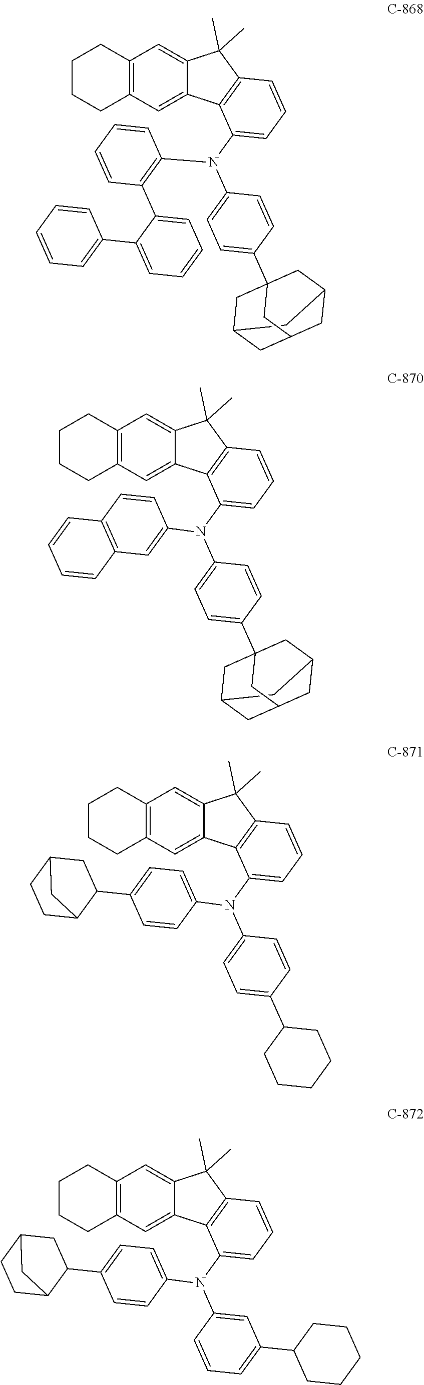

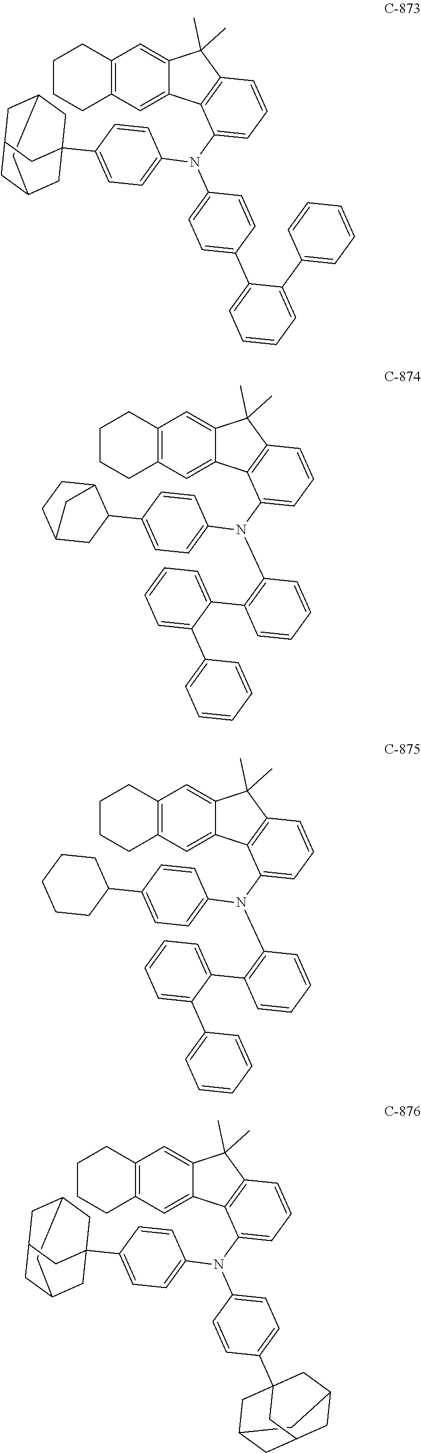

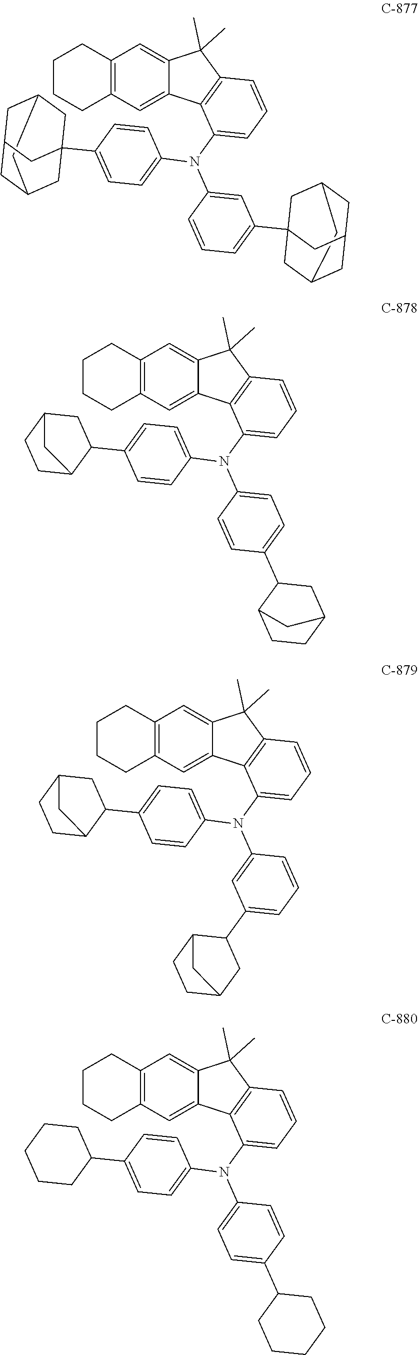

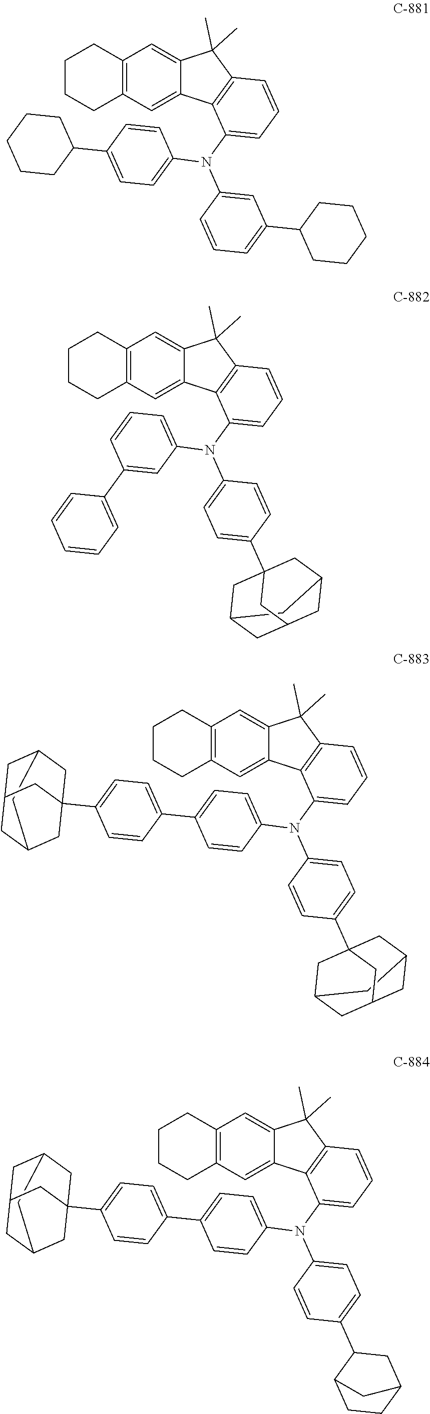 [SMILES: CC1(C)c2cc3c(cc2-c2c(N(c4ccc(C5CCCCC5)cc4)c4cccc(C5CCCCC5)c4)cccc21)CCCC3 ; CC1(C)c2cc3c(cc2-c2c(N(c4ccc(C56CC7CC(CC(C7)C5)C6)cc4)c4cccc(-c5ccccc5)c4)cccc21)CCCC3 ; CC1(C)c2cc3c(cc2-c2c(N(c4ccc(-c5ccc(C67CC8CC(CC(C8)C6)C7)cc5)cc4)c4ccc(C56CC7CC(CC(C7)C5)C6)cc4)cccc21)CCCC3 ; CC1(C)c2cc3c(cc2-c2c(N(c4ccc(-c5ccc(C67CC8CC(CC(C8)C6)C7)cc5)cc4)c4ccc(C5CC6CCC5C6)cc4)cccc21)CCCC3]

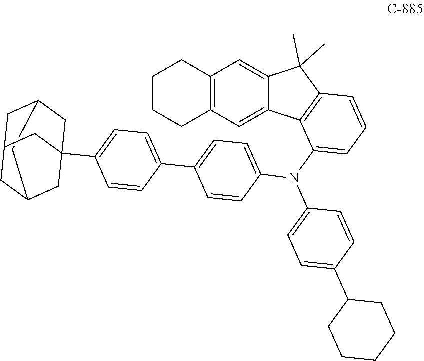 [SMILES: CC1(C)c2cc3c(cc2-c2c(N(c4ccc(-c5ccc(C67CC8CC(CC(C8)C6)C7)cc5)cc4)c4ccc(C5CCCCC5)cc4)cccc21)CCCC3]

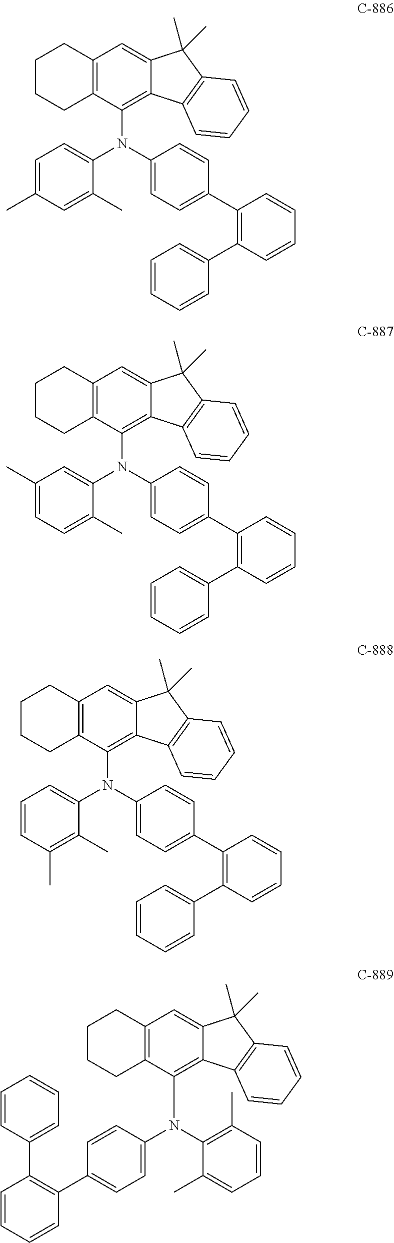

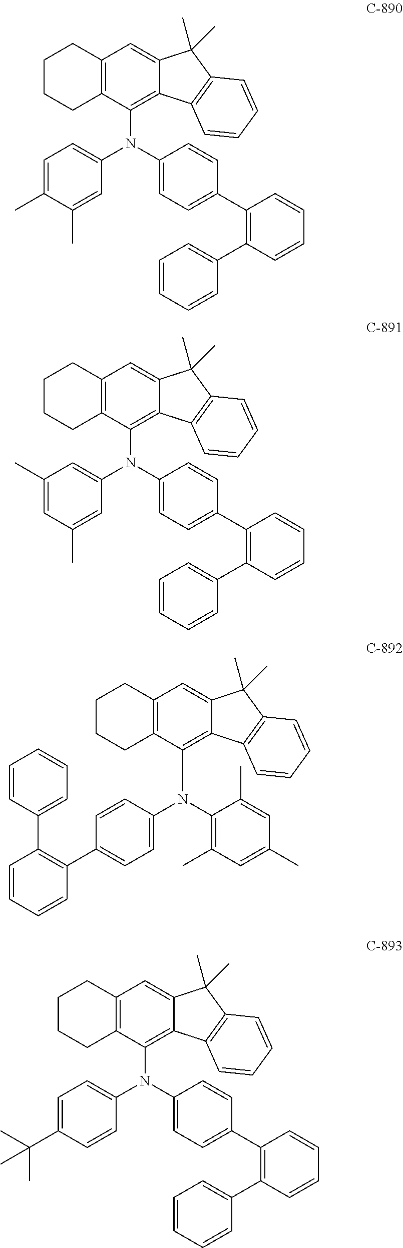

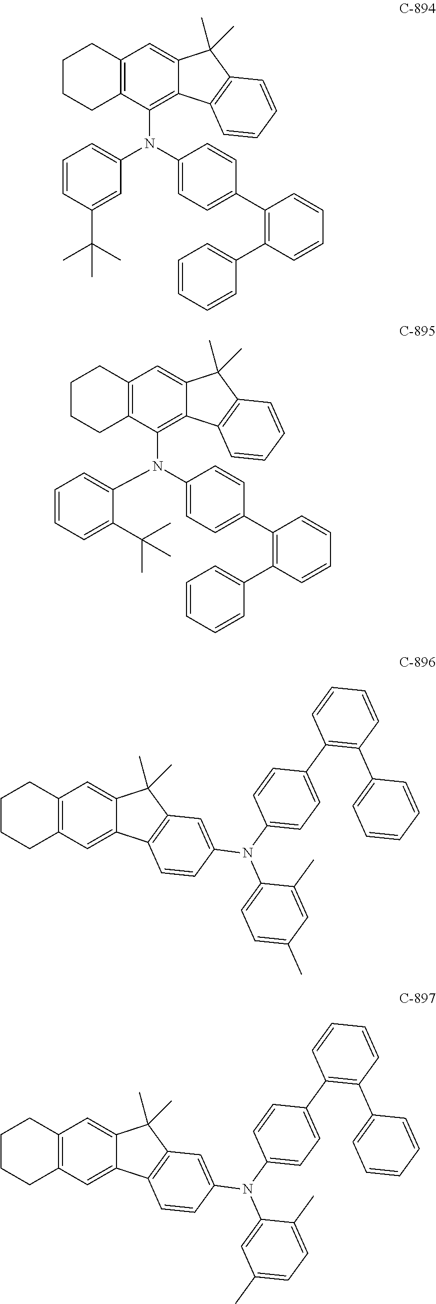

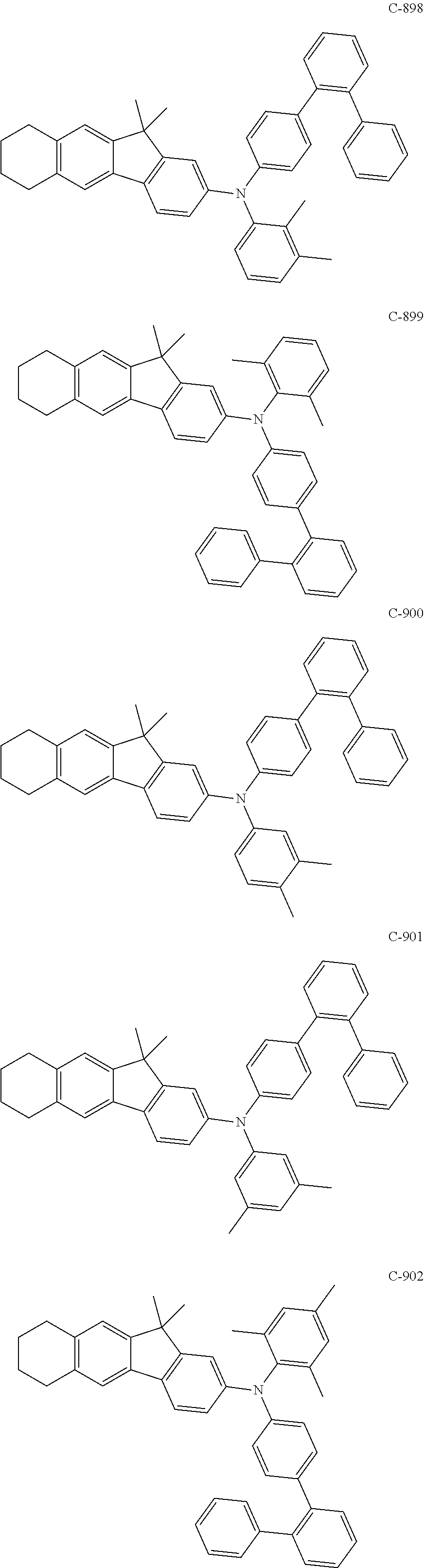

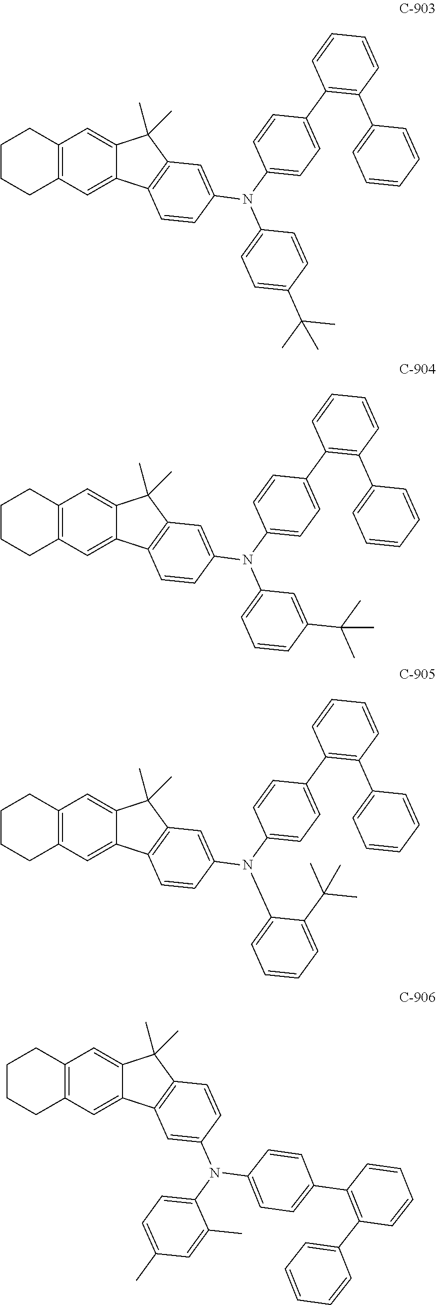

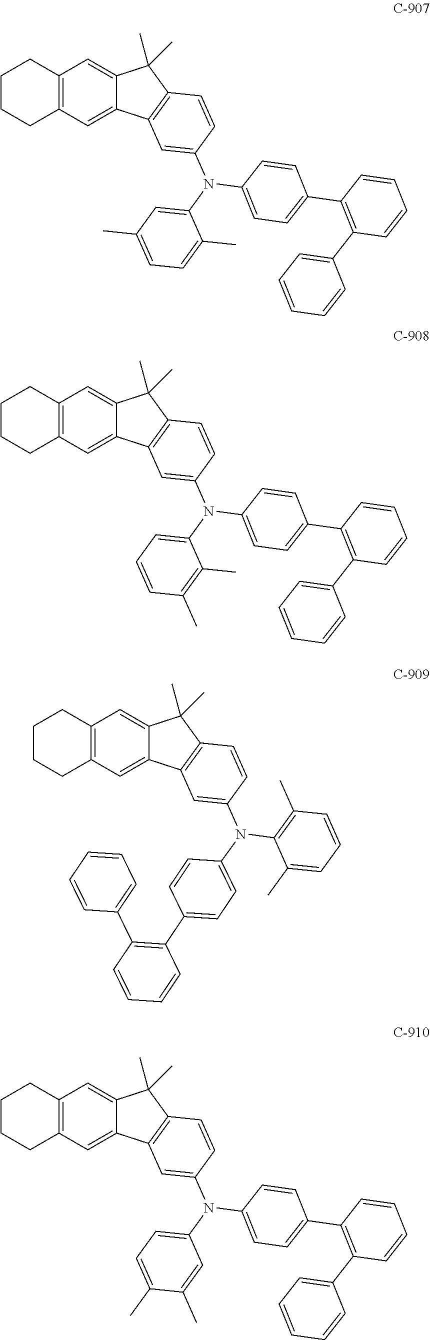 [SMILES: Cc1ccc(C)c(N(c2ccc(-c3ccccc3-c3ccccc3)cc2)c2ccc3c(c2)-c2cc4c(cc2C3(C)C)CCCC4)c1 ; Cc1cccc(N(c2ccc(-c3ccccc3-c3ccccc3)cc2)c2ccc3c(c2)-c2cc4c(cc2C3(C)C)CCCC4)c1C ; Cc1cccc(C)c1N(c1ccc(-c2ccccc2-c2ccccc2)cc1)c1ccc2c(c1)-c1cc3c(cc1C2(C)C)CCCC3 ; Cc1ccc(N(c2ccc(-c3ccccc3-c3ccccc3)cc2)c2ccc3c(c2)-c2cc4c(cc2C3(C)C)CCCC4)cc1C]

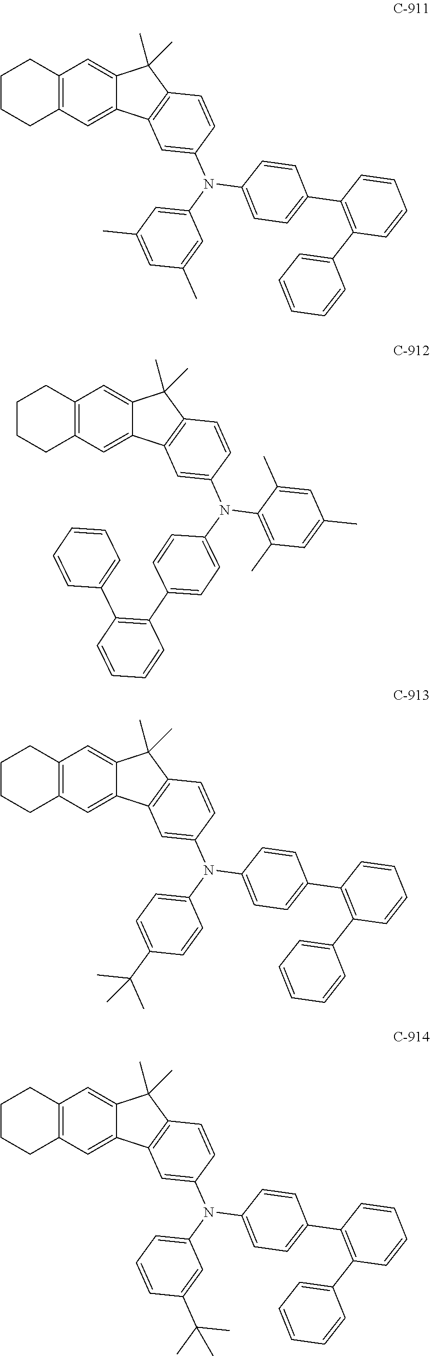

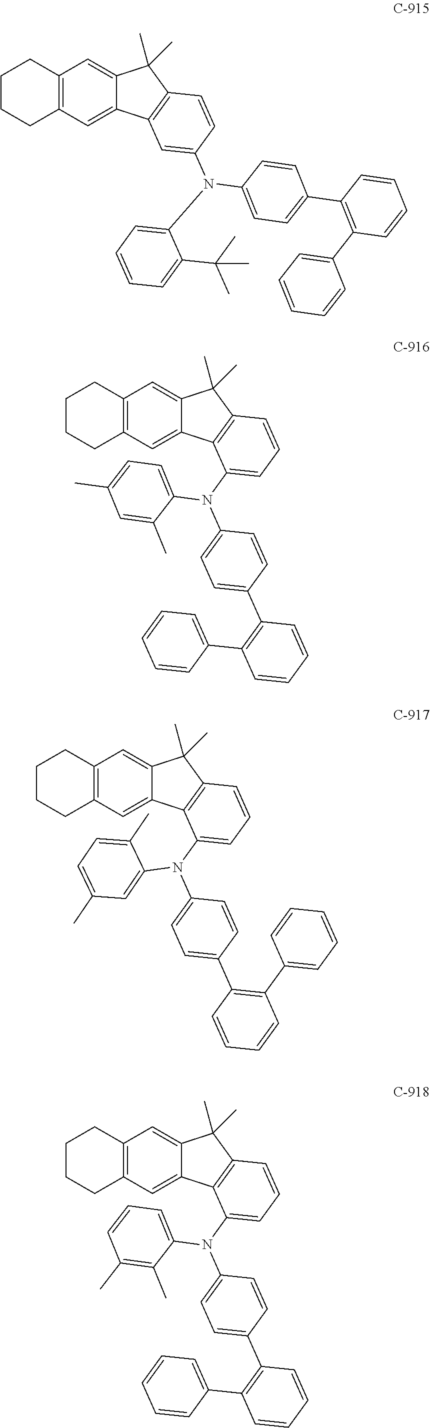 [SMILES: CC(C)(C)c1ccccc1N(c1ccc(-c2ccccc2-c2ccccc2)cc1)c1ccc2c(c1)-c1cc3c(cc1C2(C)C)CCCC3 ; Cc1ccc(N(c2ccc(-c3ccccc3-c3ccccc3)cc2)c2cccc3c2-c2cc4c(cc2C3(C)C)CCCC4)c(C)c1 ; Cc1ccc(C)c(N(c2ccc(-c3ccccc3-c3ccccc3)cc2)c2cccc3c2-c2cc4c(cc2C3(C)C)CCCC4)c1 ; Cc1cccc(N(c2ccc(-c3ccccc3-c3ccccc3)cc2)c2cccc3c2-c2cc4c(cc2C3(C)C)CCCC4)c1C]

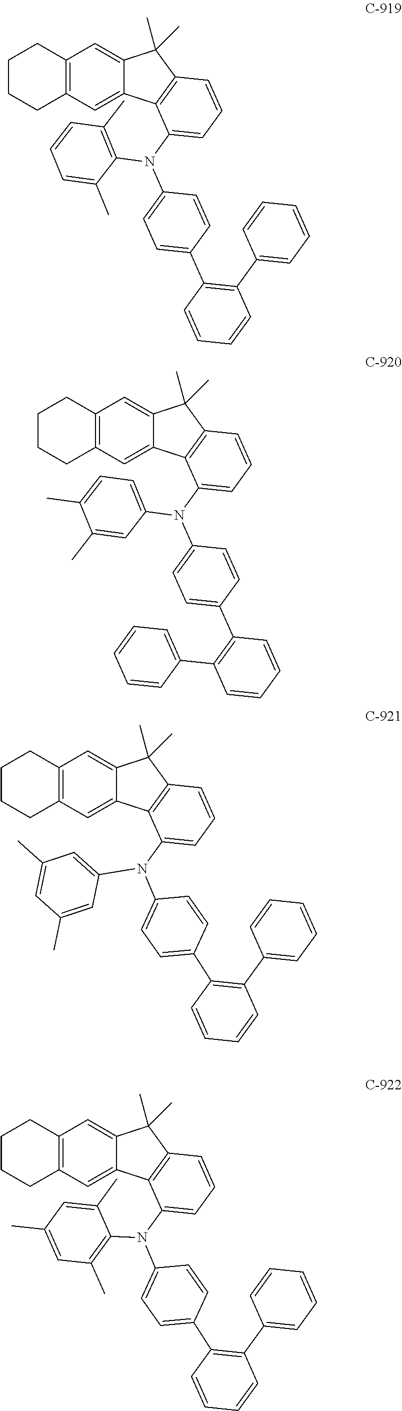

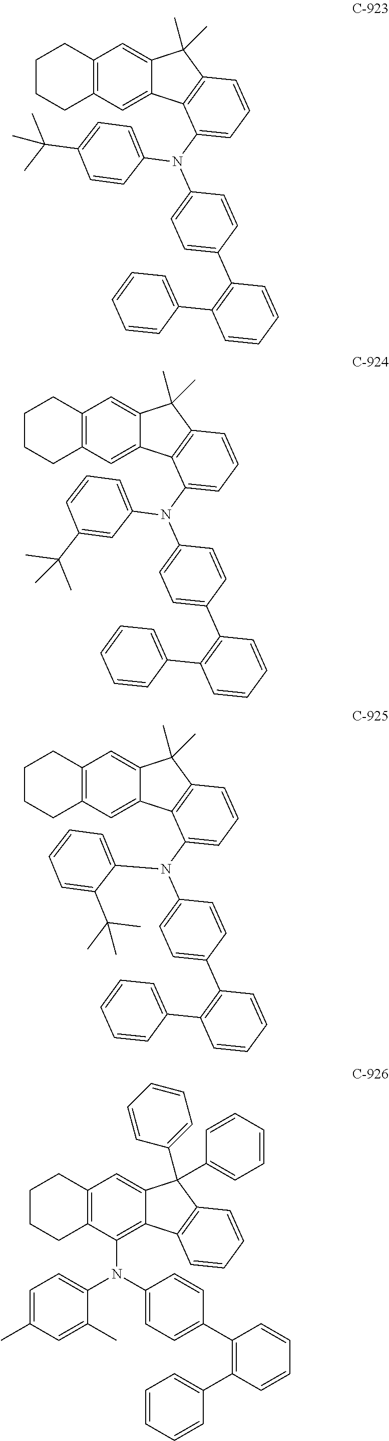

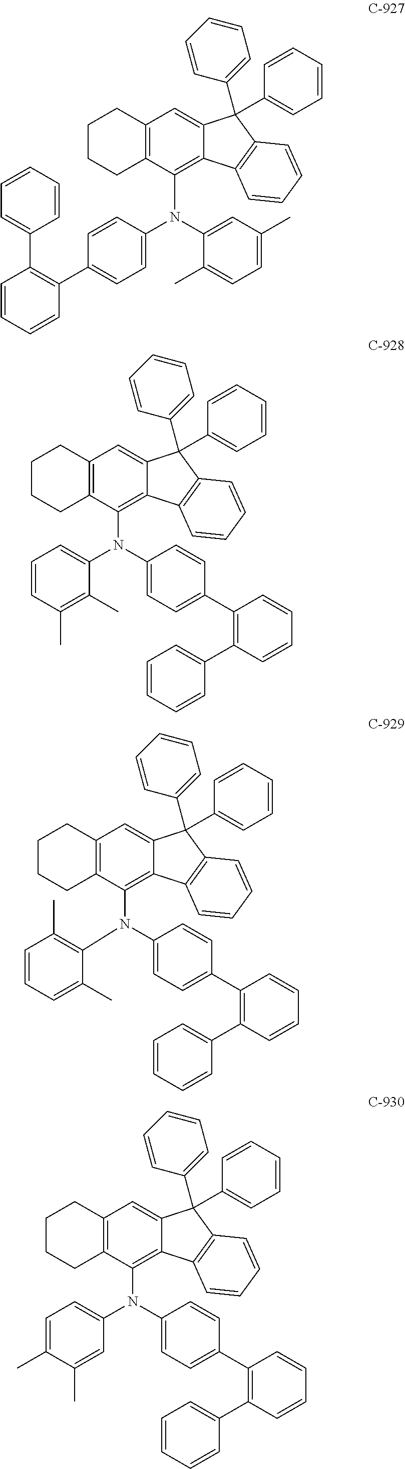

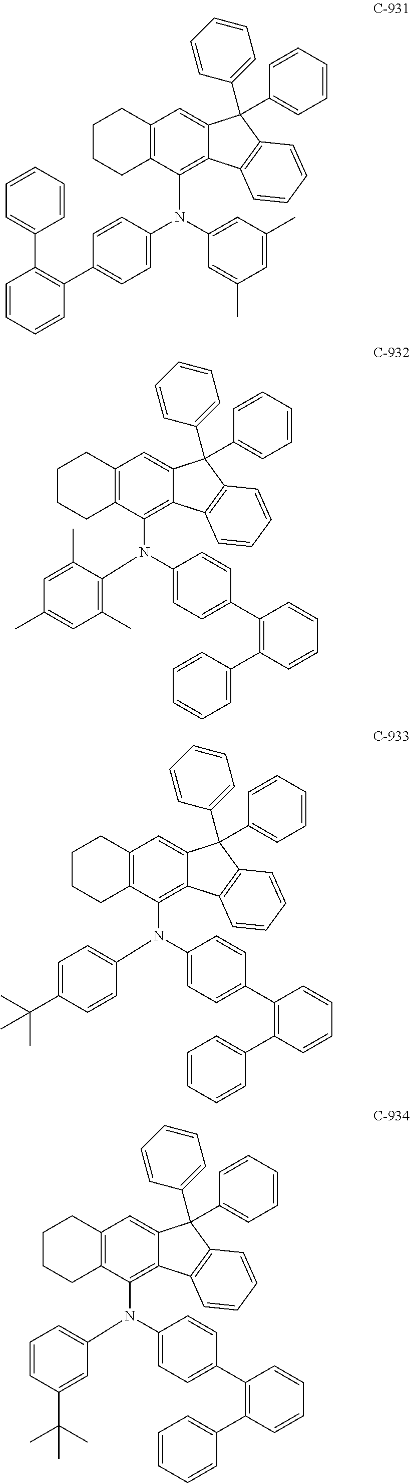

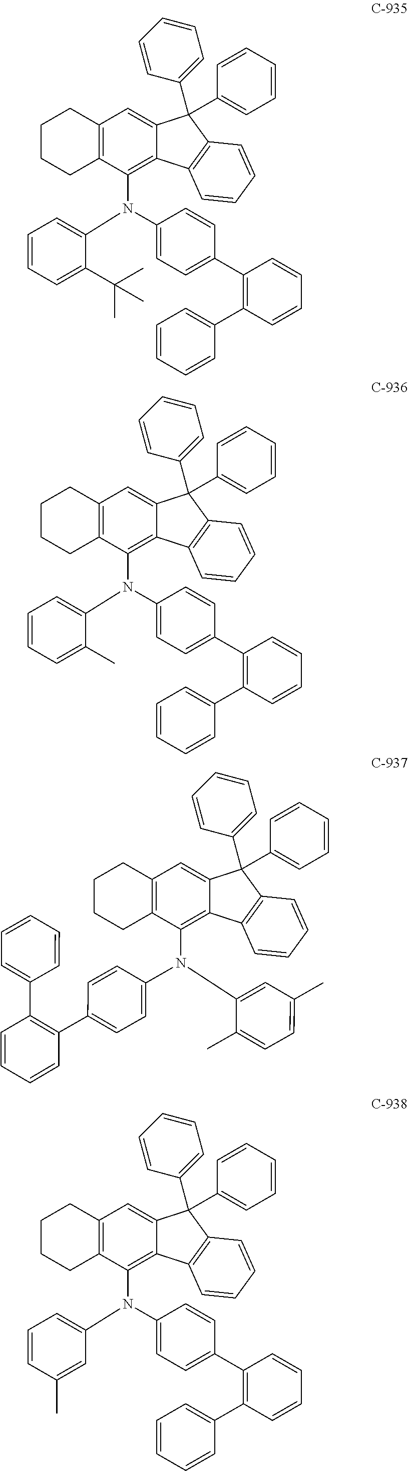

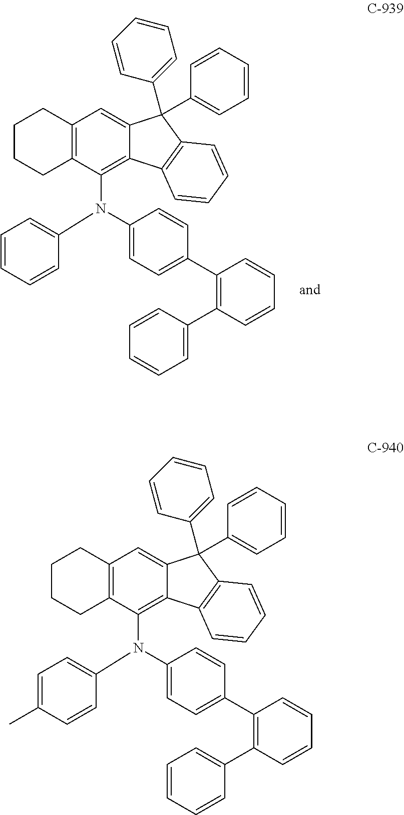 [SMILES: c1ccc(-c2ccccc2-c2ccc(N(c3ccccc3)c3c4c(cc5c3-c3ccccc3C5(c3ccccc3)c3ccccc3)CCCC4)cc2)cc1 ; Cc1ccc(N(c2ccc(-c3ccccc3-c3ccccc3)cc2)c2c3c(cc4c2-c2ccccc2C4(c2ccccc2)c2ccccc2)CCCC3)cc1]

9. An organic electroluminescent material comprising the organic electroluminescent compound according to claim 1.

10. An organic electroluminescent device comprising the organic electroluminescent compound according to claim 1.

11. An organic electroluminescent device according to claim 10, wherein the device comprises an organic electroluminescent compound in at least one layer selected from a hole transport layer, a light-emitting layer, a hole auxiliary layer, an electron-blocking layer, and a light-emitting auxiliary layer.

12. The organic electroluminescent device comprising a first electrode; a second electrode facing the first electrode; a light-emitting layer between the first electrode and the second electrode; and a hole transport zone between the first electrode and the light-emitting layer;
wherein the hole transport zone comprises the compound represented by Formula 1 of claim 1;
wherein the light-emitting layer comprises a compound represented by the following Formula 6 and a compound represented by the following Formula 7: 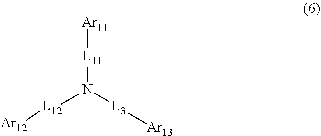
wherein in Formula 6,
L₁₁ to L₁₃ each independently represent a single bond, a substituted or unsubstituted (C6-C30)arylene, or a substituted or unsubstituted (3- to 30-membered)heteroarylene;
Ar₁₁ represents a substituted or unsubstituted (3- to 30-membered)heteroaryl;
Ar₁₂ and Ar₁₃ each independently represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C2-C30)alkenylamino, a substituted or unsubstituted (C1-C30)alkyl(C2-C30)alkenylamino, a substituted or unsubstituted mono- or di(C6-C30)arylamino, a substituted or unsubstituted (C1-C30)alkyl(C6-C30)arylamino, a substituted or unsubstituted mono- or di(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; 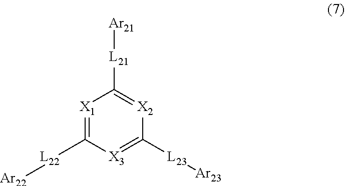
wherein in Formula 7,
X₁ to X₃ each independently represent N or CR₂₁; with the proviso that at least one of X₁ to X₃ is N;
each R₂₁ independently represents hydrogen or deuterium;
L₂₁ to L₂₃ each independently represent a single bond, a substituted or unsubstituted (C6-C30)arylene, or a substituted or unsubstituted (3- to 30-membered)heteroarylene; and
Ar₂₁ to Ar₂₃ each independently represent a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl;
with the proviso that at least one of Ar₂₁ to Ar₂₃ is a substituted or unsubstituted (3- to 30-membered)heteroaryl.

13. The organic electroluminescent device according to claim 12, wherein Ar₁₁ of Formula 6 is a substituted or unsubstituted (3- to 30-membered) heteroaryl having four or more fused rings and necessarily containing an oxygen atom.

14. The organic electroluminescent device according to claim 12, wherein Ar₁₁ of Formula 6 is represented by the following Formula 6-1 or 6-2: 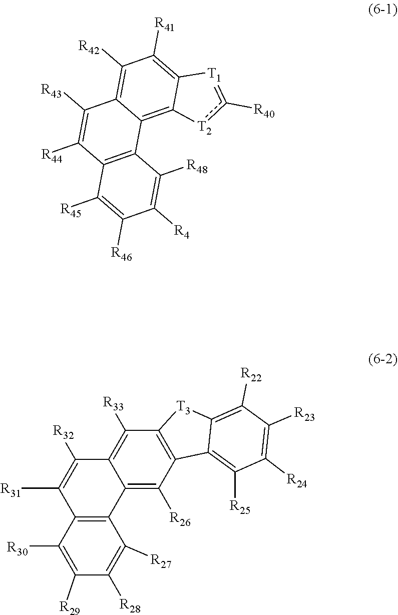
wherein in Formulas 6-1 and 6-2,
T₁ and T₂ each independently represent —N═, —NR₂₀—, —O—, or —S—, with the proviso that one of T₁ and T₂ is —N═, and the other of T₁ and T₂ is —NR₂₀—, —O—, or —S—;
T₃ represents O or S;
R₄₀ represents a substituted or unsubstituted (C6-C30)aryl, or a substituted or unsubstituted (3- to 30-membered)heteroaryl; and
R₄₁ to R₄₈ and R₂₂ to R₃₃ are each independently linked to 11, or represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di(C1-C30)alkylamino, a substituted or unsubstituted mono- or di(C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di(C6-C30)arylamino, a substituted or unsubstituted mono- or di-(3- to 30-membered)heteroarylamino, or a substituted or unsubstituted (C6-C30)aryl(3- to 30-membered)heteroarylamino, or may be linked to an adjacent substituent(s) to form a ring(s);
with the proviso that in Formula 6-1, any one of R₄₁ to R₄₈ is linked to 11,
and in Formula 6-2, any one of R₂₂ to R₃₃ is linked to 11.

15. The organic electroluminescent device according to claim 12, wherein Formula 7 is represented by any one of the following Formulas 7-1 to 7-4: 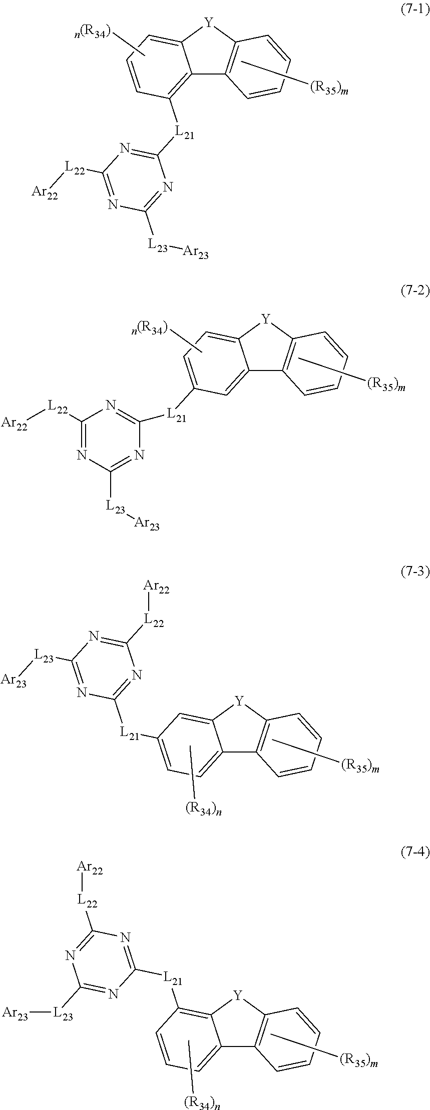
wherein in Formulas 7-1 to 7-4,
Y represents O, S, or NR₃₆;
R₃₄ to R₃₆ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di(C1-C30)alkylamino, a substituted or unsubstituted mono- or di(C6-C30)arylamino, or a substituted or unsubstituted (C1-C30)alkyl(C6-C30)arylamino; or may be linked to an adjacent substituent(s) to form a ring(s);
L₂₁ to L₂₃, Ar₂₂, and Ar₂₃ are as defined in claim 12;
n represents an integer of 1 to 3, m represents an integer of 1 to 4; and
if n and m are integers of 2 or more, each R₃₄ and each R₃₅ may be the same as or different from each other.

16. The organic electroluminescent device according to claim 12, wherein the compound represented by Formula 6 is selected from the following compounds: 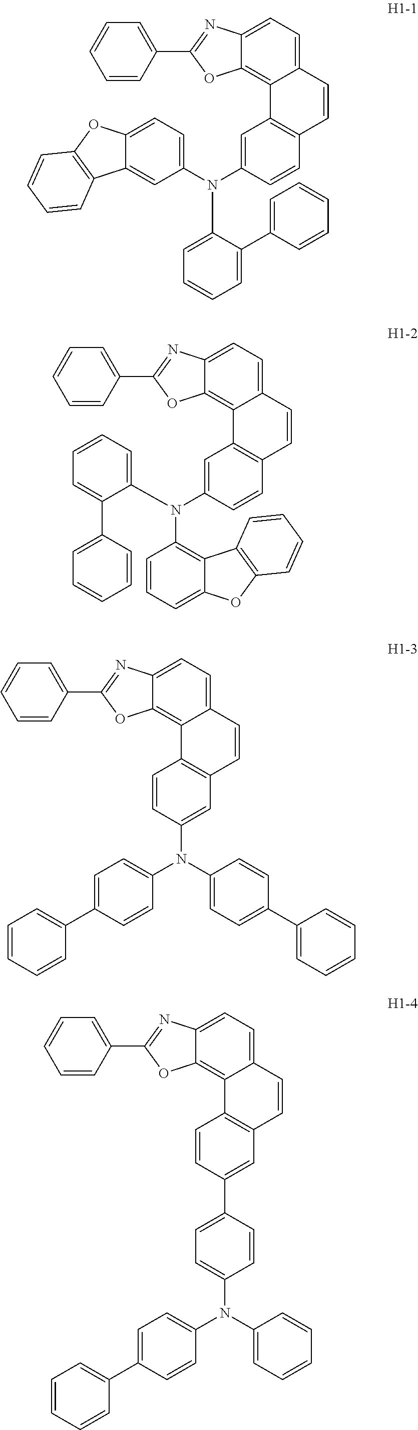 [SMILES: c1ccc(-c2nc3ccc4ccc5ccc(N(c6ccc7oc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1 ; c1ccc(-c2ccc(N(c3ccccc3)c3ccc(-c4ccc5c(ccc6ccc7nc(-c8ccccc8)oc7c65)c4)cc3)cc2)cc1] 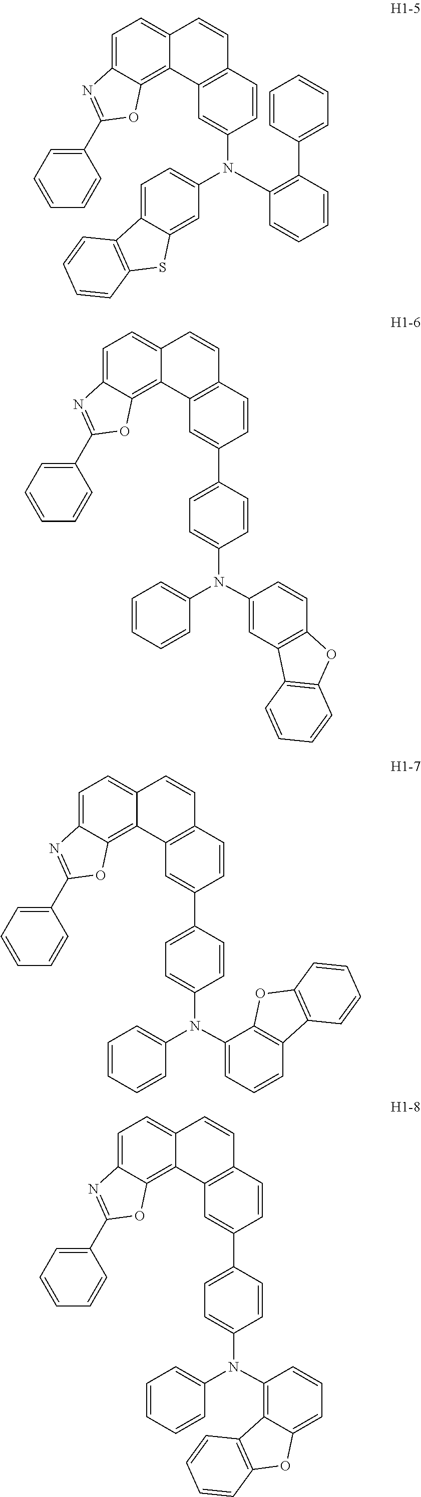 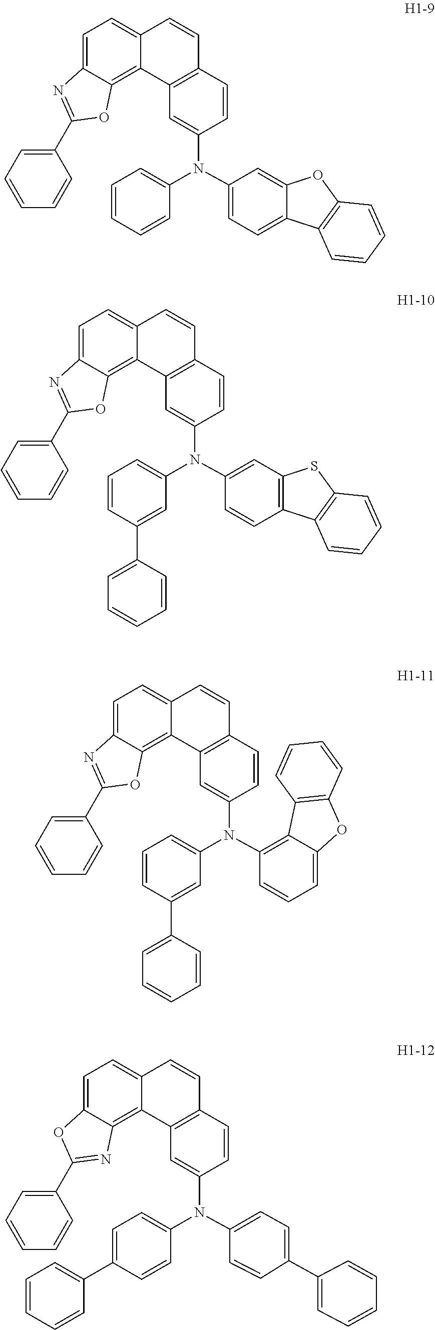 [SMILES: c1ccc(-c2nc3ccc4ccc5ccc(N(c6ccccc6)c6ccc7c(c6)oc6ccccc67)cc5c4c3o2)cc1 ; c1ccc(-c2cccc(N(c3ccc4c(c3)sc3ccccc34)c3ccc4ccc5ccc6nc(-c7ccccc7)oc6c5c4c3)c2)cc1 ; c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1] 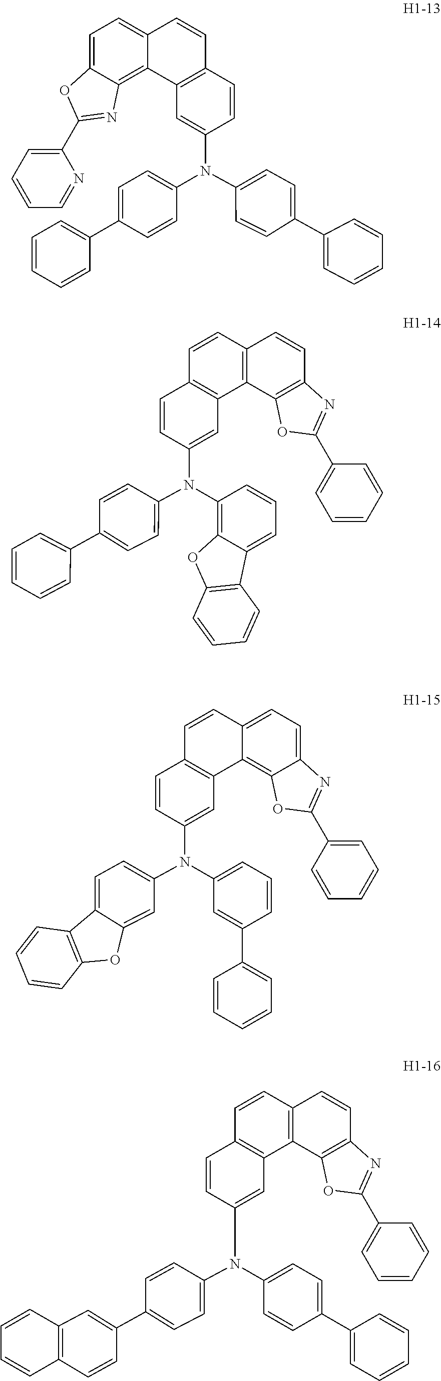 [SMILES: c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1 ; c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc(-c4ccc5ccccc5c4)cc3)c3ccc4ccc5ccc6nc(-c7ccccc7)oc6c5c4c3)cc2)cc1] 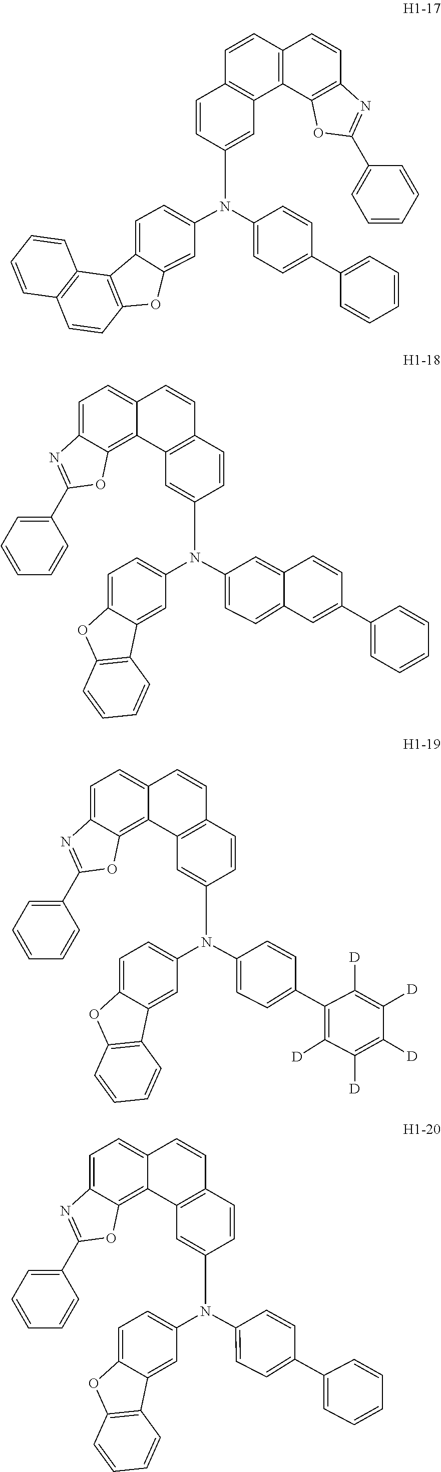 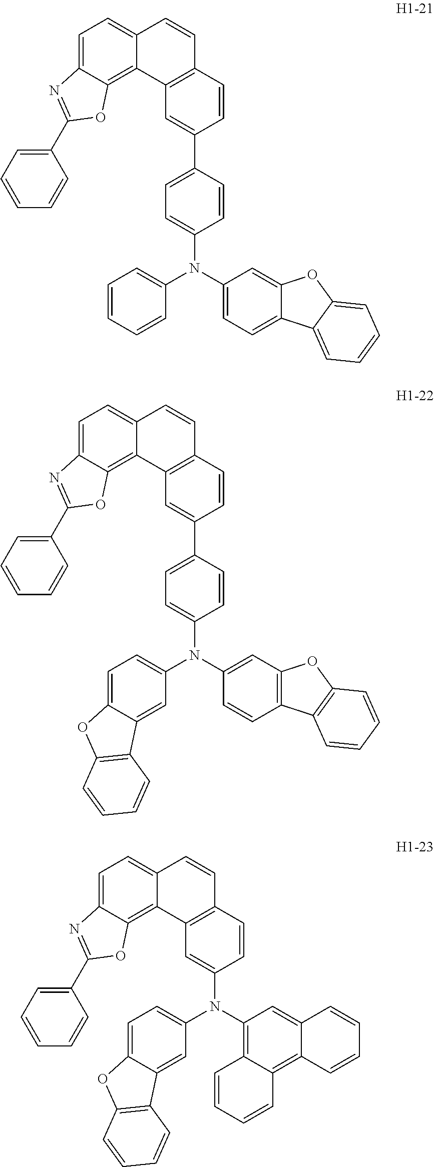 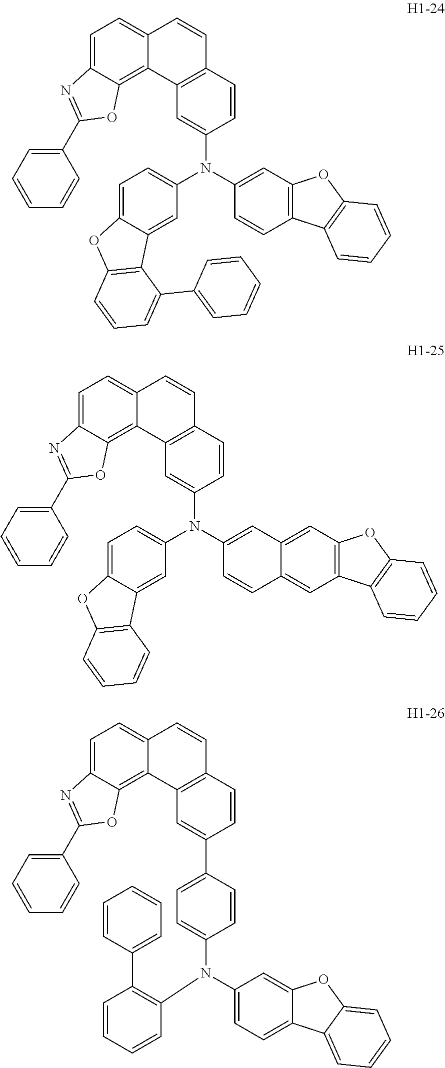 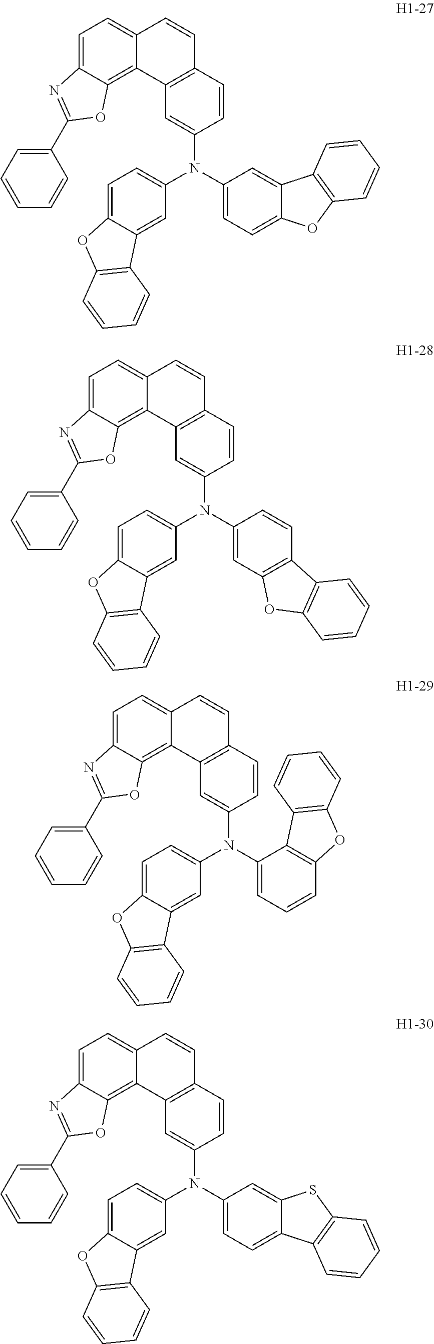 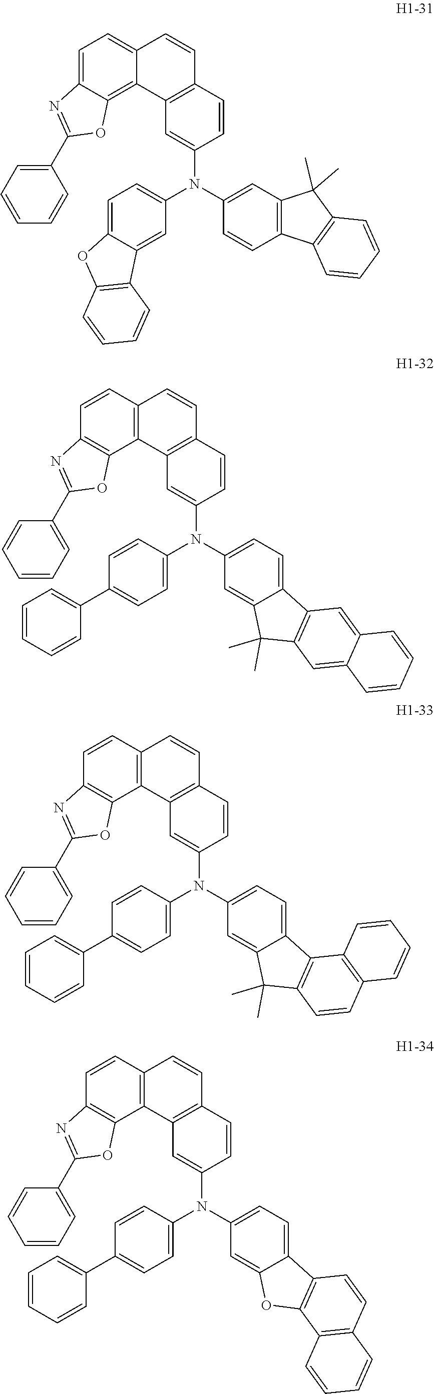 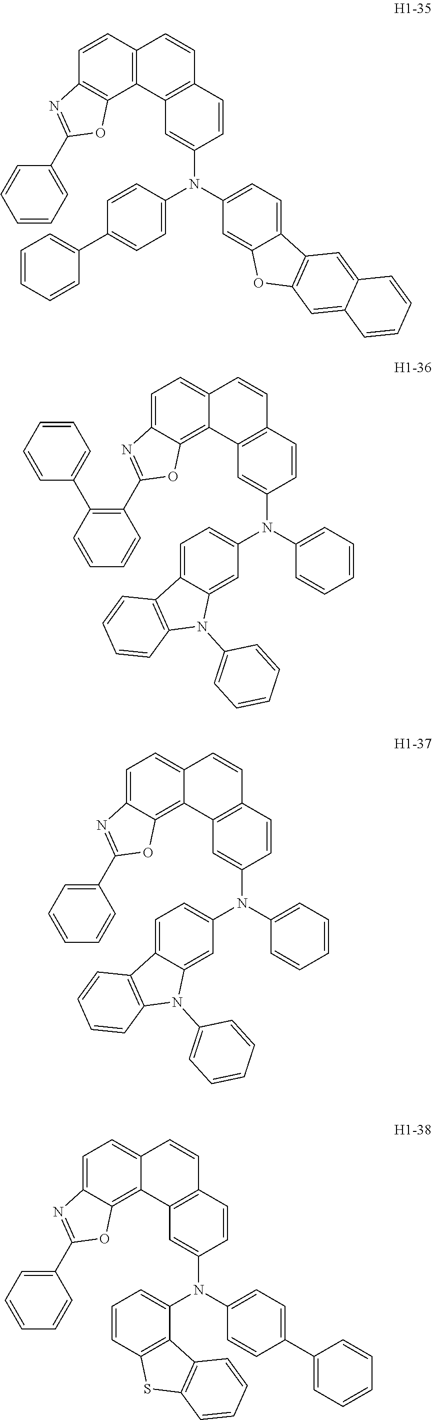 [SMILES: c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccccc2-c2nc3ccc4ccc5ccc(N(c6ccccc6)c6ccc7c8ccccc8n(-c8ccccc8)c7c6)cc5c4c3o2)cc1 ; c1ccc(-c2nc3ccc4ccc5ccc(N(c6ccccc6)c6ccc7c8ccccc8n(-c8ccccc8)c7c6)cc5c4c3o2)cc1 ; c1ccc(-c2ccc(N(c3ccc4ccc5ccc6nc(-c7ccccc7)oc6c5c4c3)c3cccc4sc5ccccc5c34)cc2)cc1] 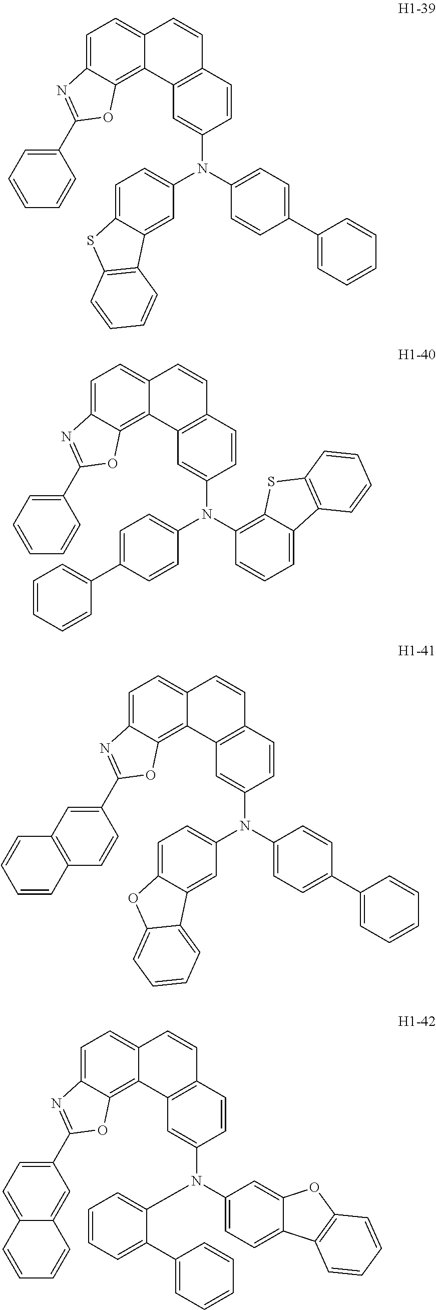 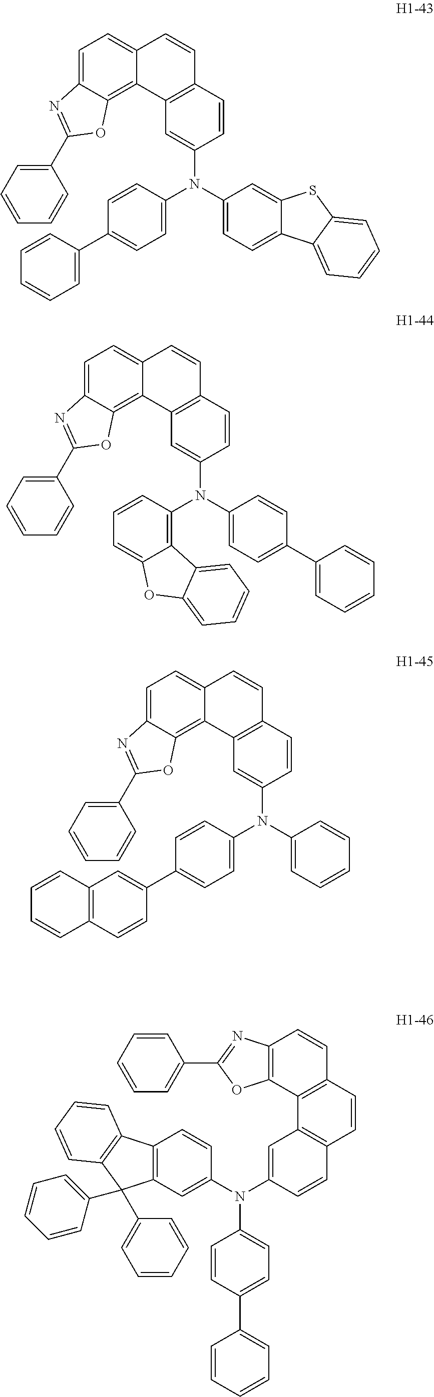 [SMILES: c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4oc5ccccc5c34)cc2)cc1 ; c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4c(c3)C(c3ccccc3)(c3ccccc3)c3ccccc3-4)c3ccc4ccc5ccc6nc(-c7ccccc7)oc6c5c4c3)cc2)cc1] 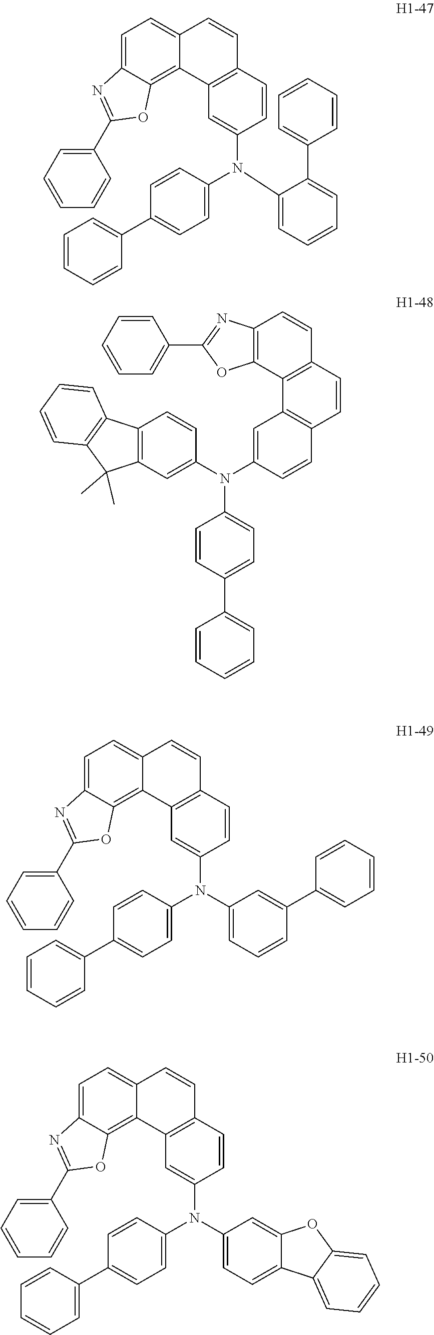 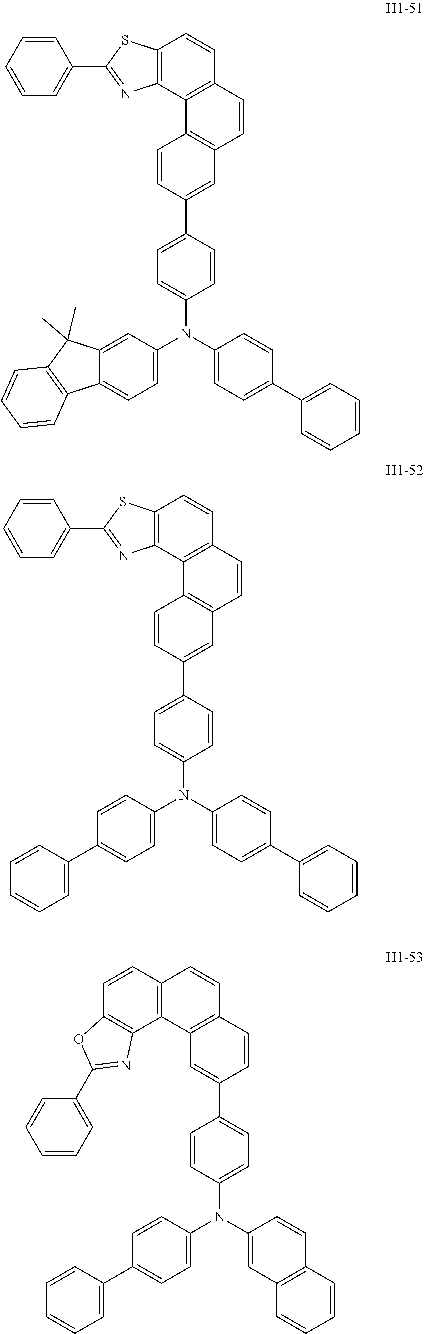 [SMILES: CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1] 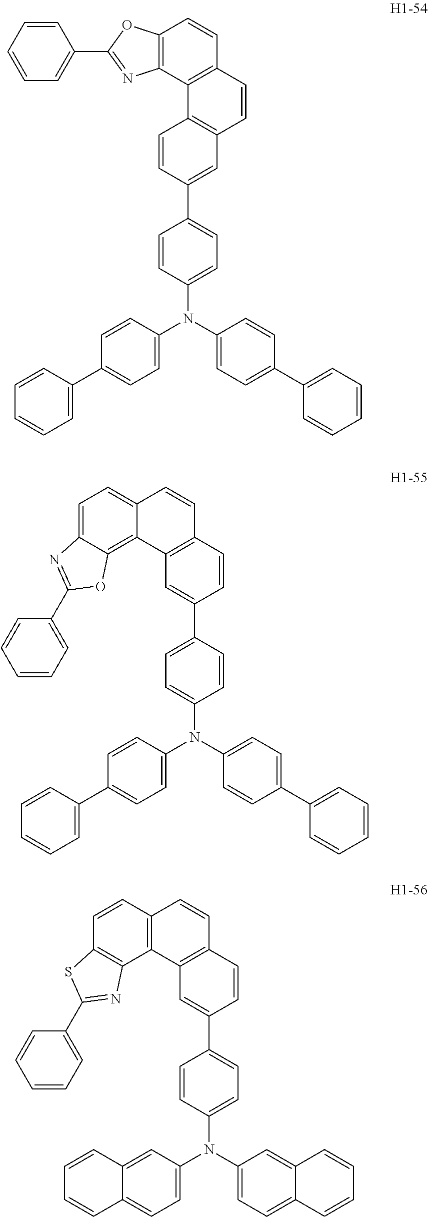 [SMILES: c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2nc3c(ccc4ccc5ccc(-c6ccc(N(c7ccc8ccccc8c7)c7ccc8ccccc8c7)cc6)cc5c43)s2)cc1] 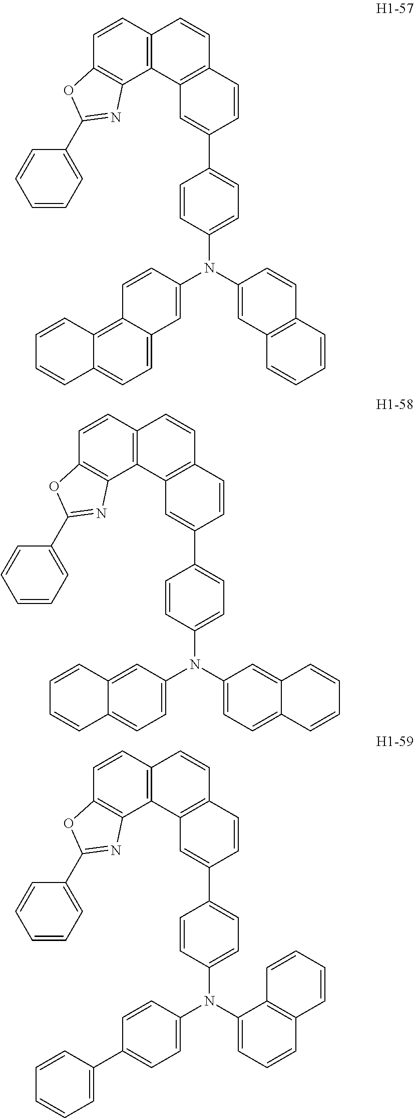 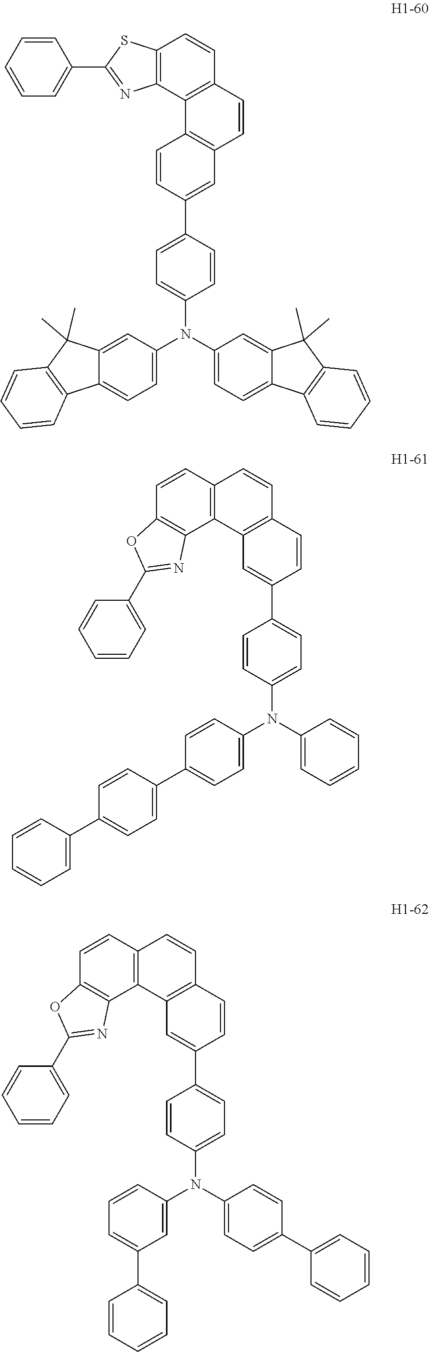 [SMILES: CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1] 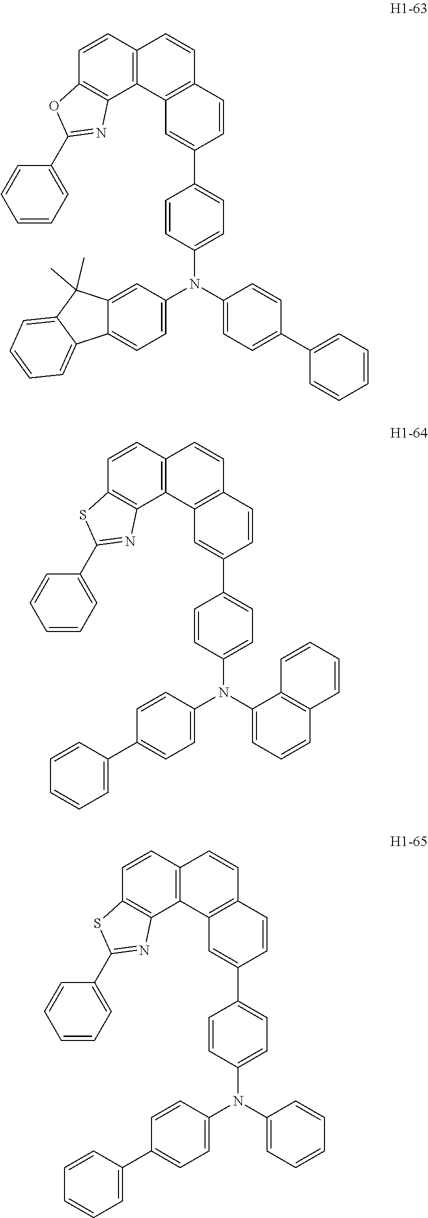 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7sc(-c8ccccc8)nc7c6c5c4)cc3)c3cccc4ccccc34)cc2)cc1 ; c1ccc(-c2ccc(N(c3ccccc3)c3ccc(-c4ccc5ccc6ccc7sc(-c8ccccc8)nc7c6c5c4)cc3)cc2)cc1] 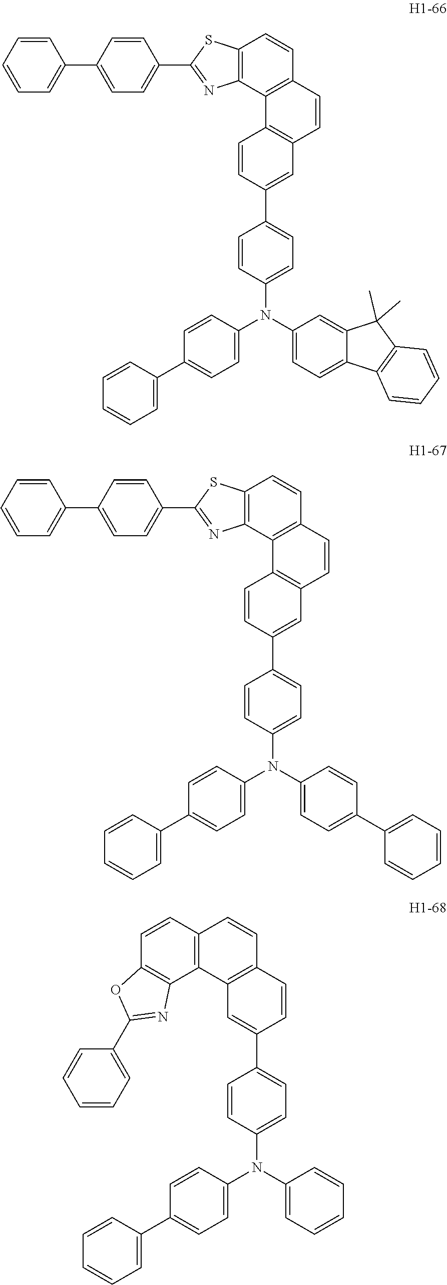 [SMILES: CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; c1ccc(-c2ccc(N(c3ccccc3)c3ccc(-c4ccc5ccc6ccc7oc(-c8ccccc8)nc7c6c5c4)cc3)cc2)cc1] 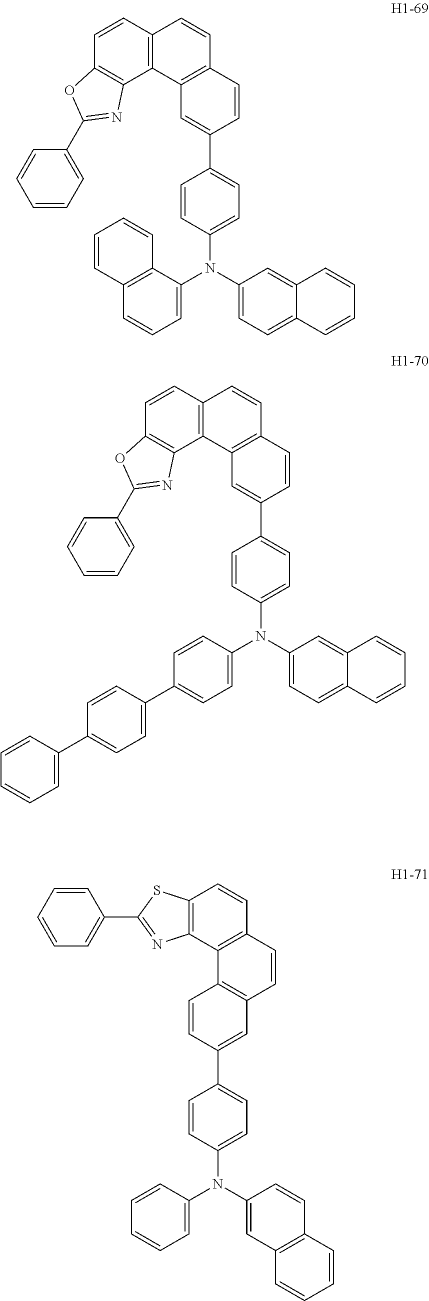 [SMILES: c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5cc(-c6ccc(N(c7ccccc7)c7ccc8ccccc8c7)cc6)ccc5c43)s2)cc1] 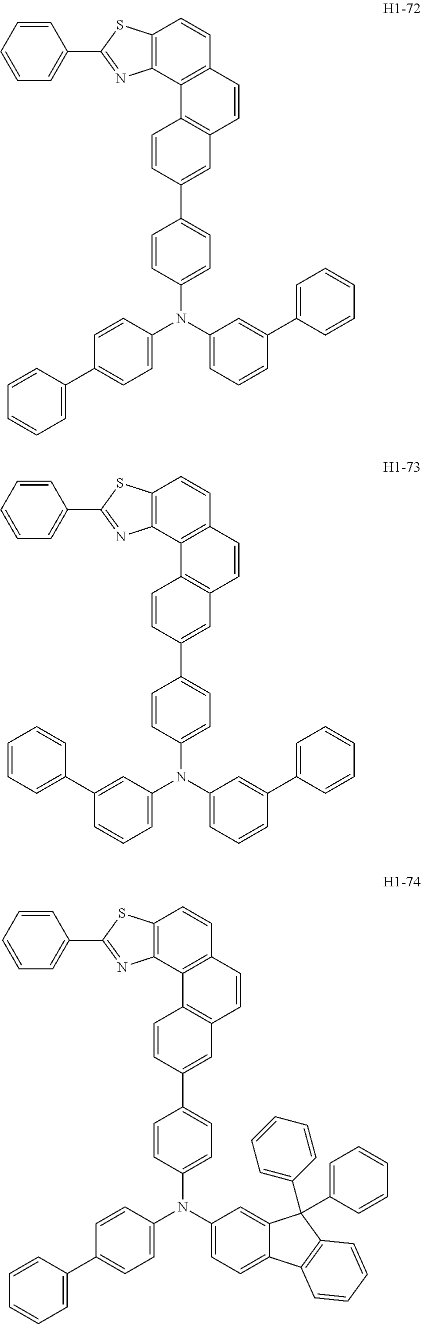 [SMILES: c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1] 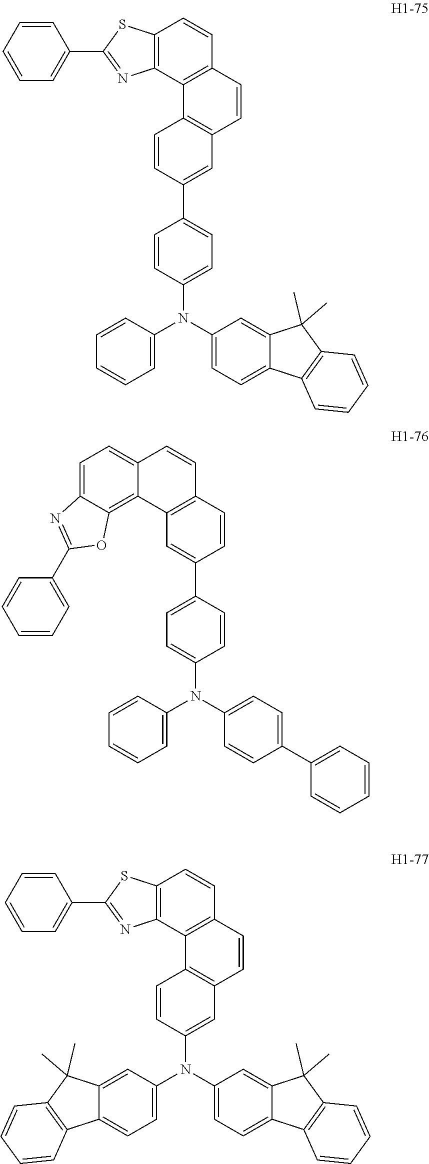 [SMILES: CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccccc3)c3ccc(-c4ccc5ccc6ccc7nc(-c8ccccc8)oc7c6c5c4)cc3)cc2)cc1 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21] 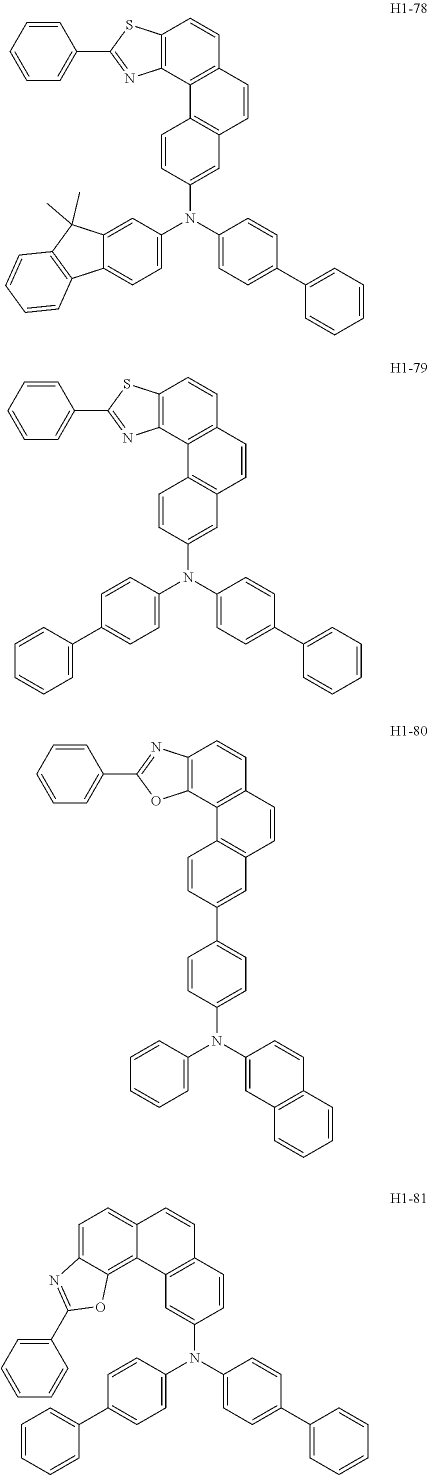 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; c1ccc(-c2ccc(N(c3ccc(-c4ccccc4)cc3)c3ccc4c(ccc5ccc6sc(-c7ccccc7)nc6c54)c3)cc2)cc1 ; c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1] 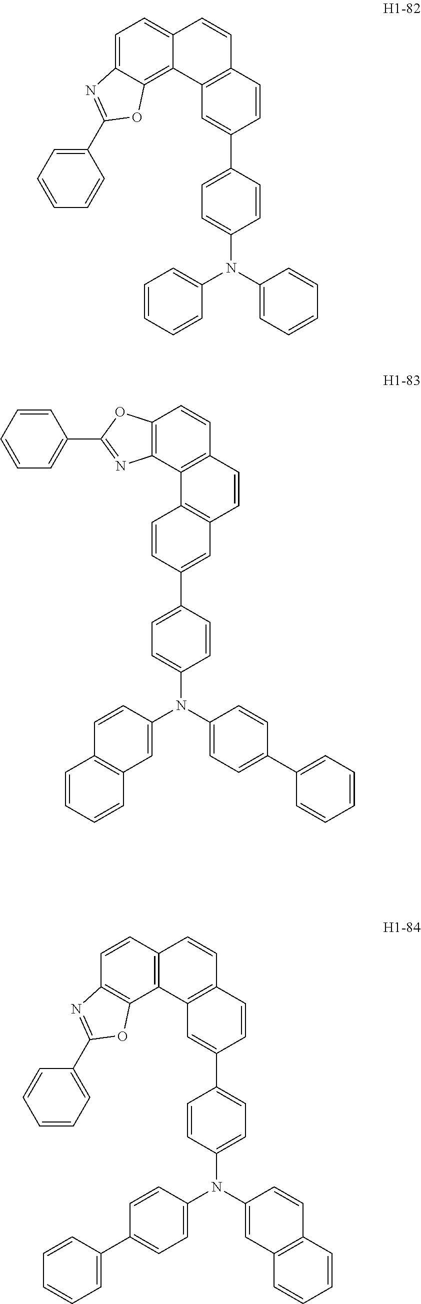 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2ccc(N(c3ccc(-c4ccc5ccc6ccc7nc(-c8ccccc8)oc7c6c5c4)cc3)c3ccc4ccccc4c3)cc2)cc1] 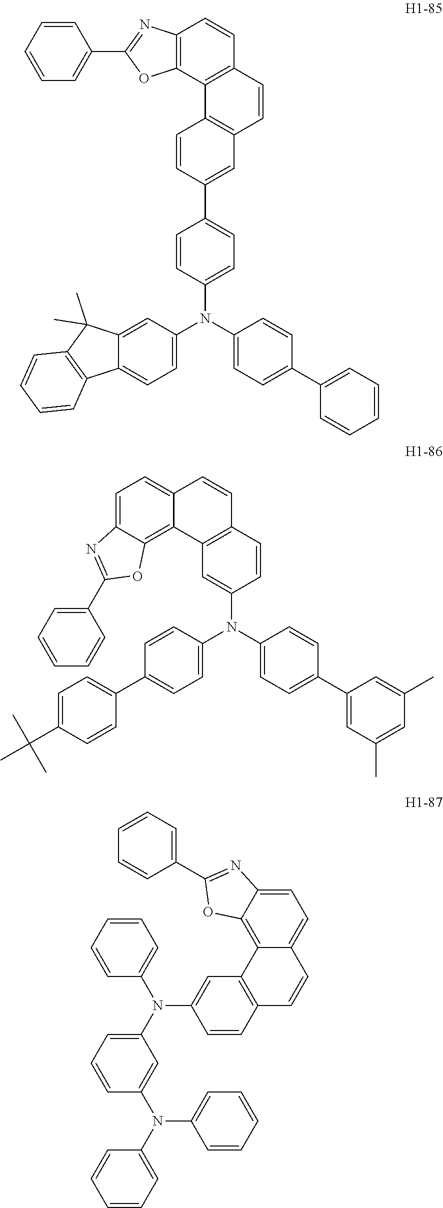 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5ccc(N(c6ccccc6)c6cccc(N(c7ccccc7)c7ccccc7)c6)cc5c4c3o2)cc1] 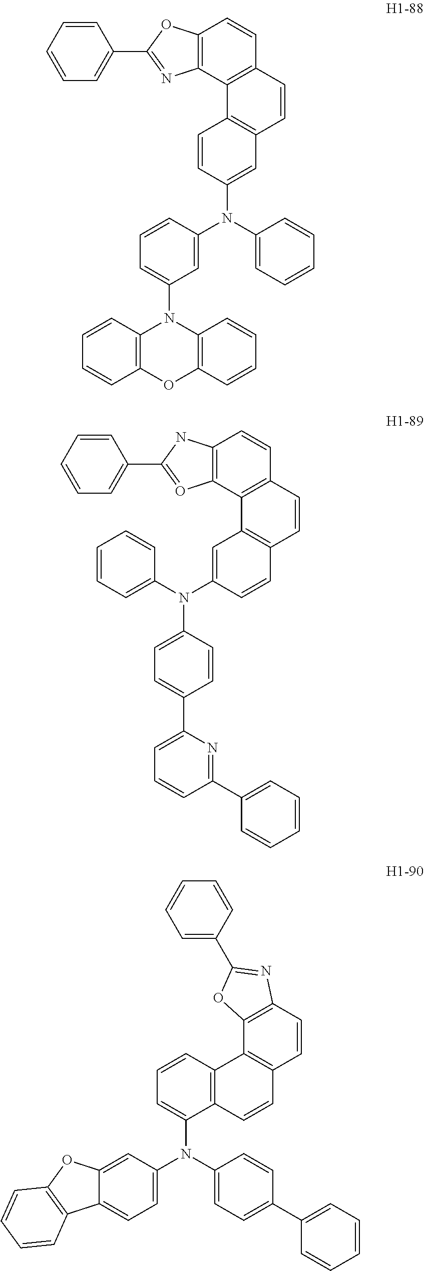 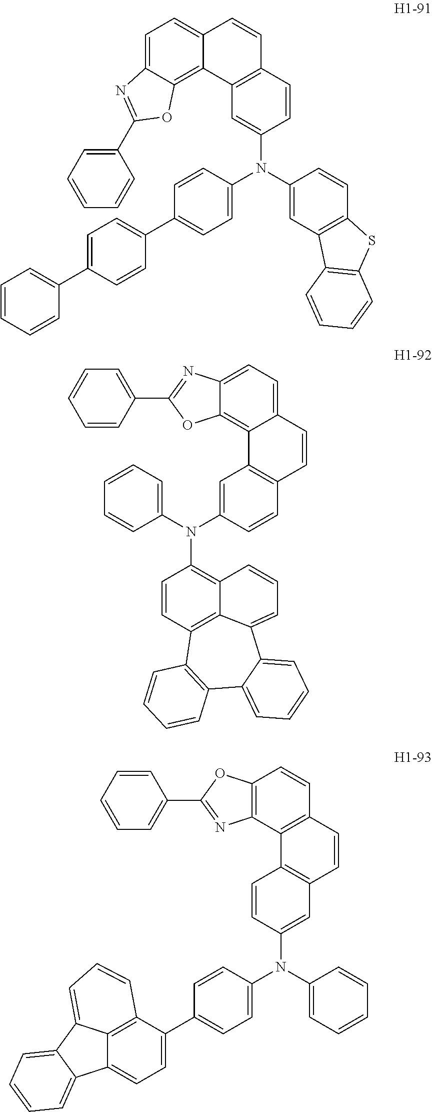 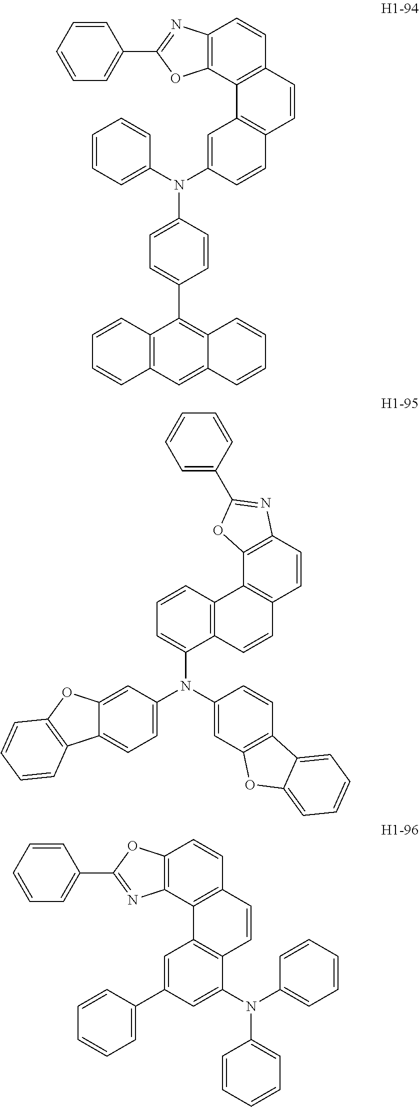 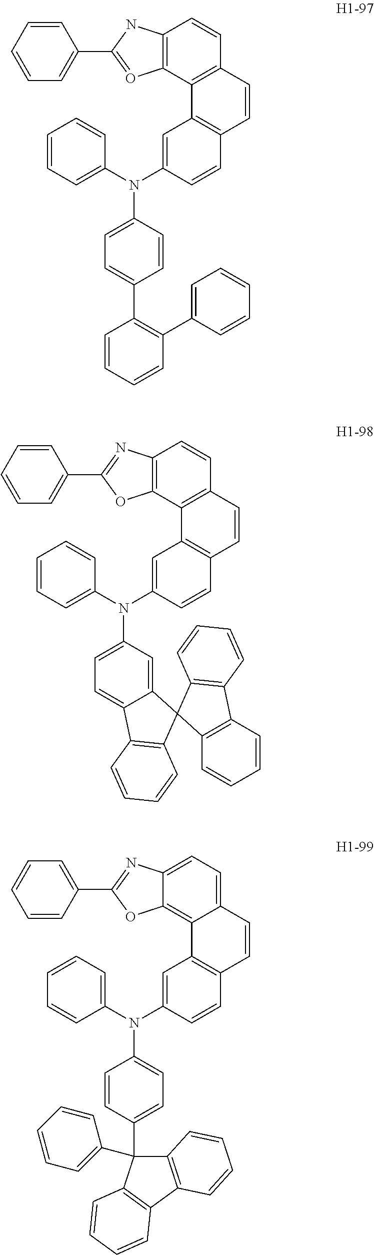 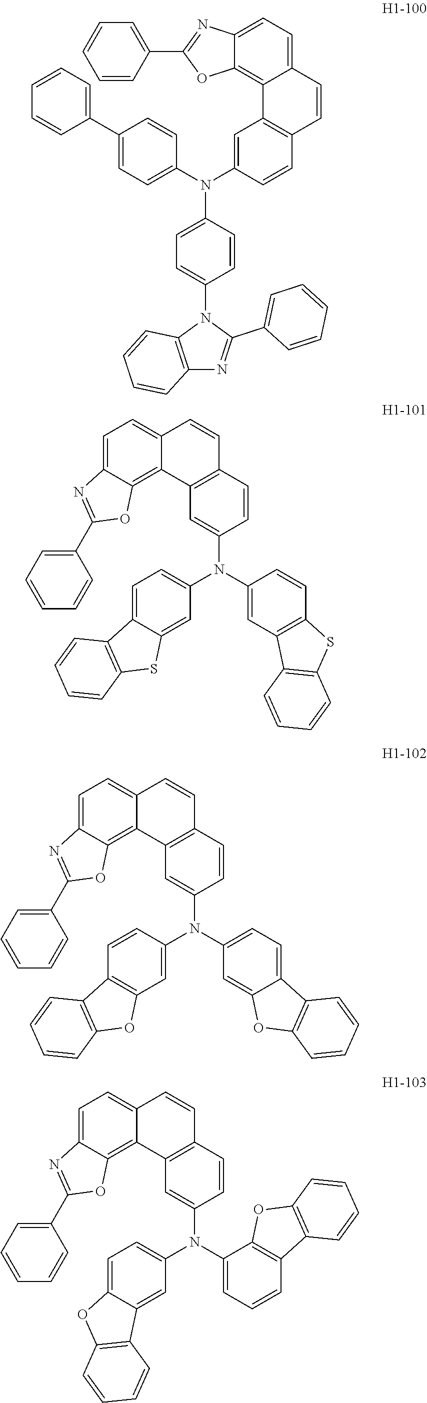 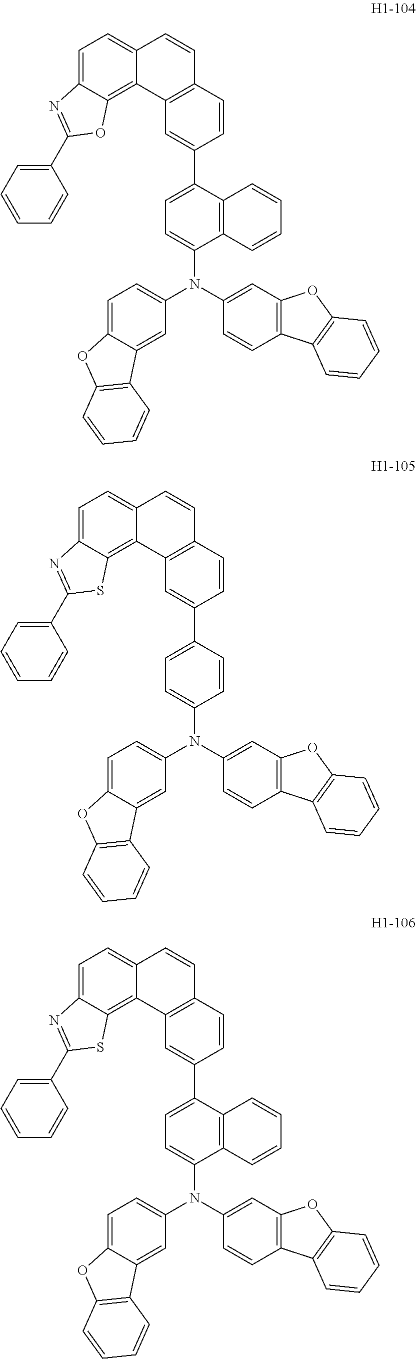 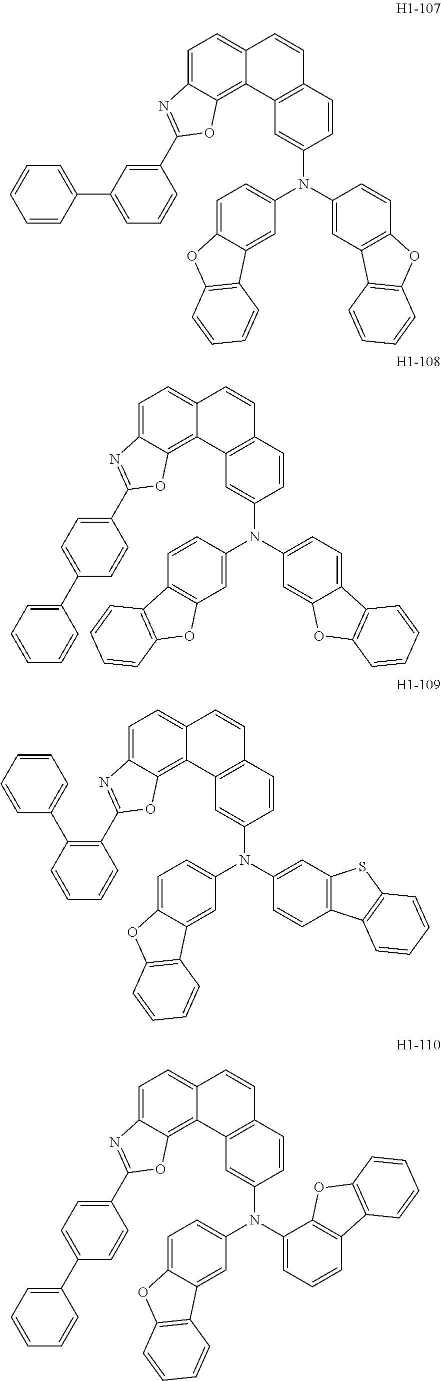 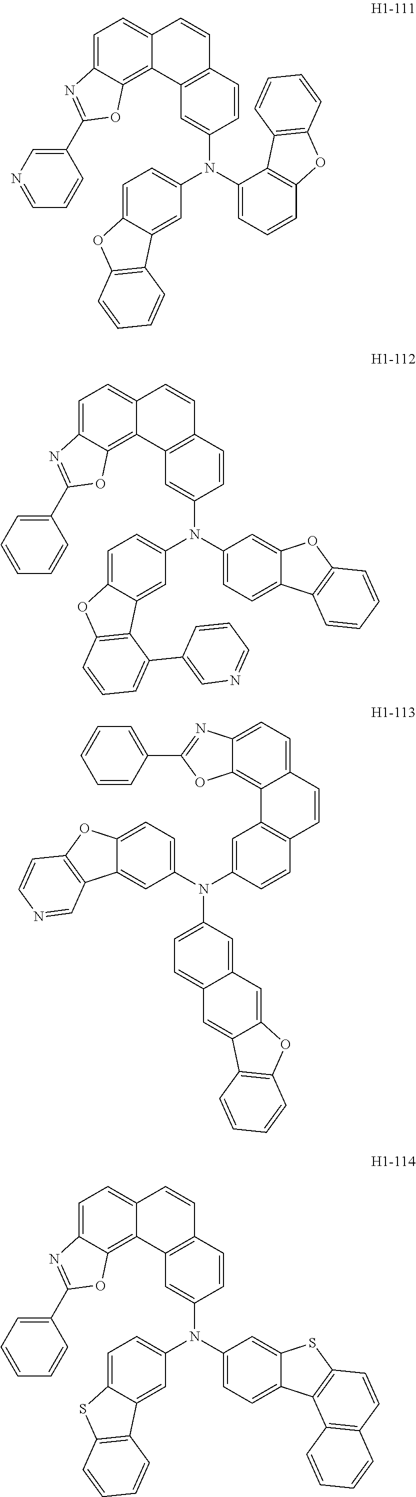 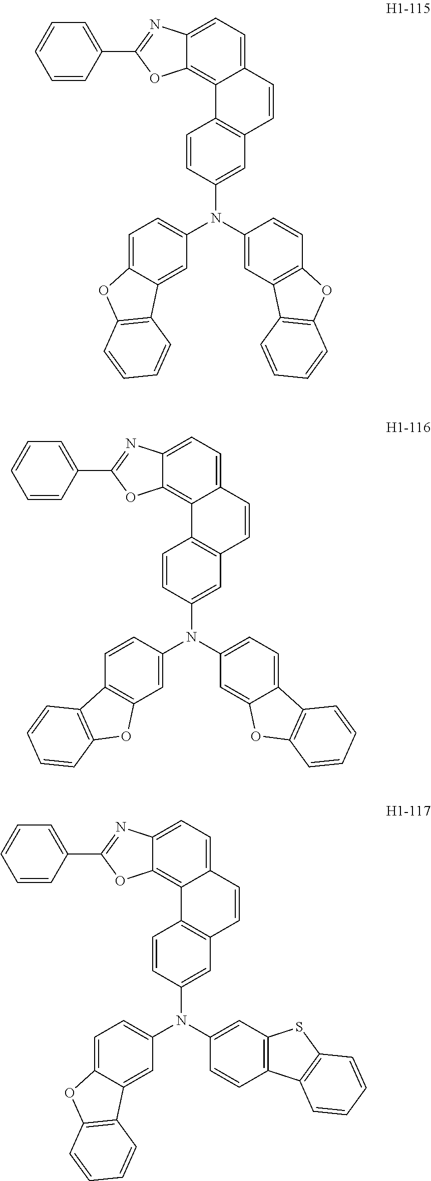 [SMILES: c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1 ; c1ccc(-c2nc3ccc4ccc5cc(N(c6ccc7c(c6)sc6ccccc67)c6ccc7oc8ccccc8c7c6)ccc5c4c3o2)cc1] 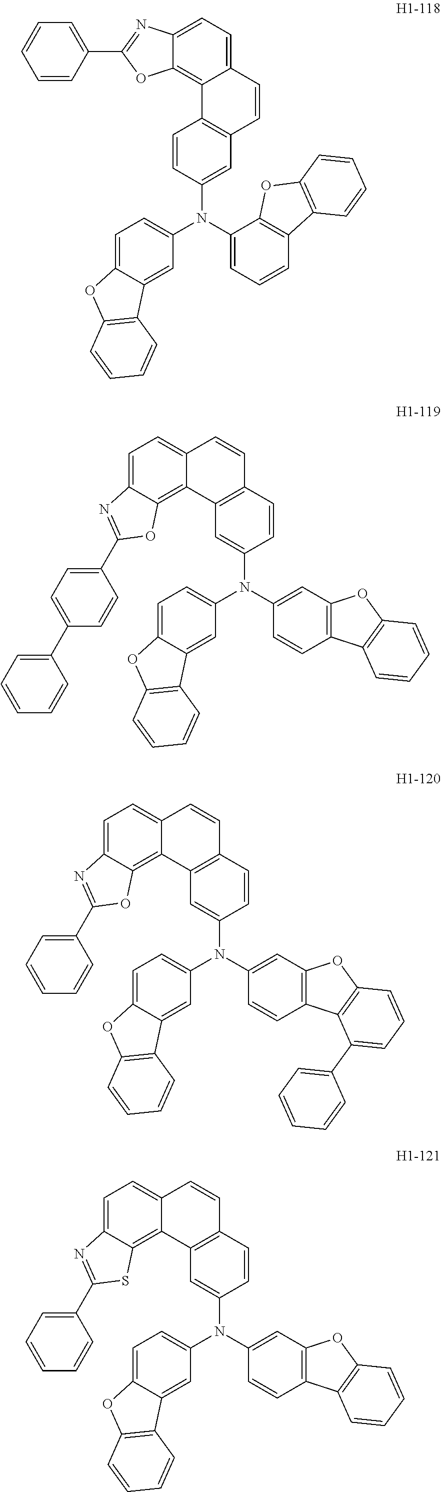 [SMILES: c1ccc(-c2nc3ccc4ccc5cc(N(c6ccc7oc8ccccc8c7c6)c6cccc7c6oc6ccccc67)ccc5c4c3o2)cc1 ; c1ccc(-c2ccc(-c3nc4ccc5ccc6ccc(N(c7ccc8c(c7)oc7ccccc78)c7ccc8oc9ccccc9c8c7)cc6c5c4o3)cc2)cc1 ; c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1] 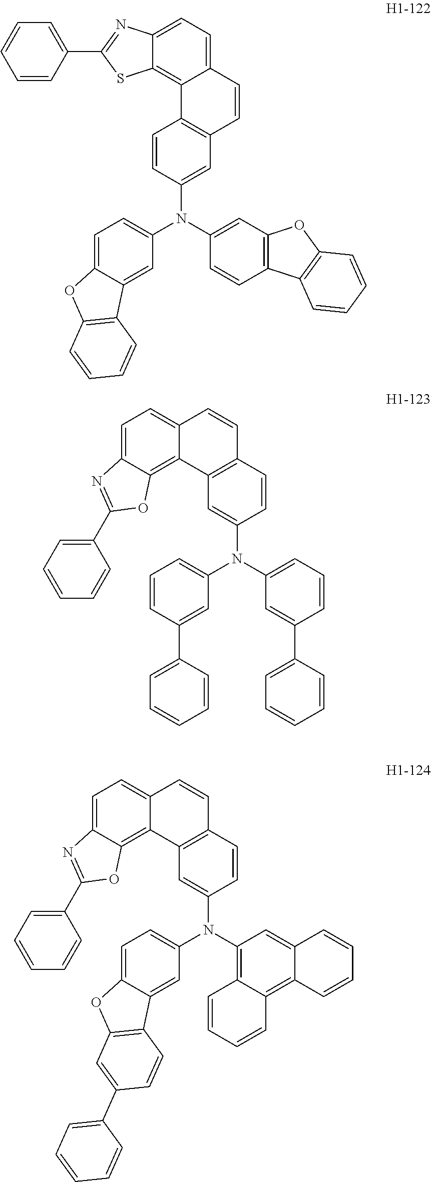 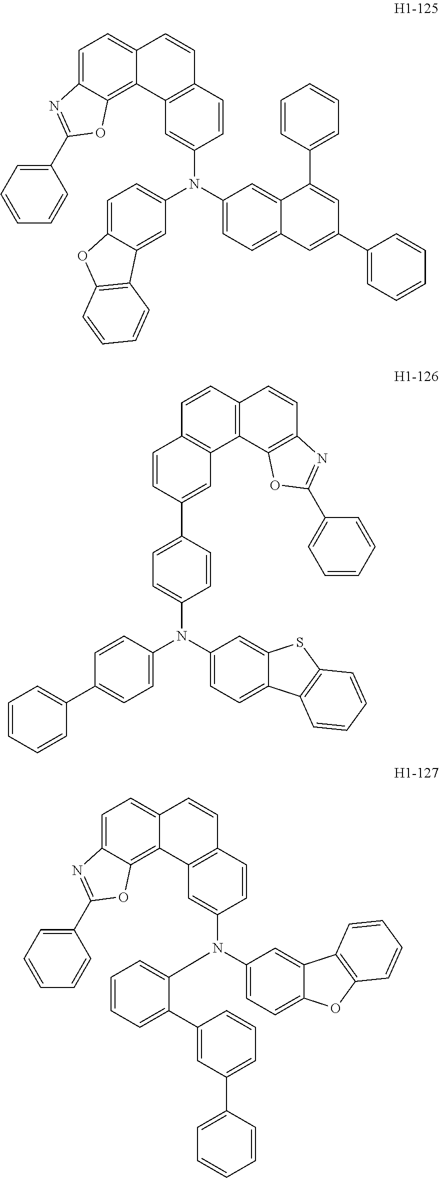 [SMILES: c1ccc(-c2cc(-c3ccccc3)c3cc(N(c4ccc5oc6ccccc6c5c4)c4ccc5ccc6ccc7nc(-c8ccccc8)oc7c6c5c4)ccc3c2)cc1 ; c1ccc(-c2ccc(N(c3ccc(-c4ccc5ccc6ccc7nc(-c8ccccc8)oc7c6c5c4)cc3)c3ccc4c(c3)sc3ccccc34)cc2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1] 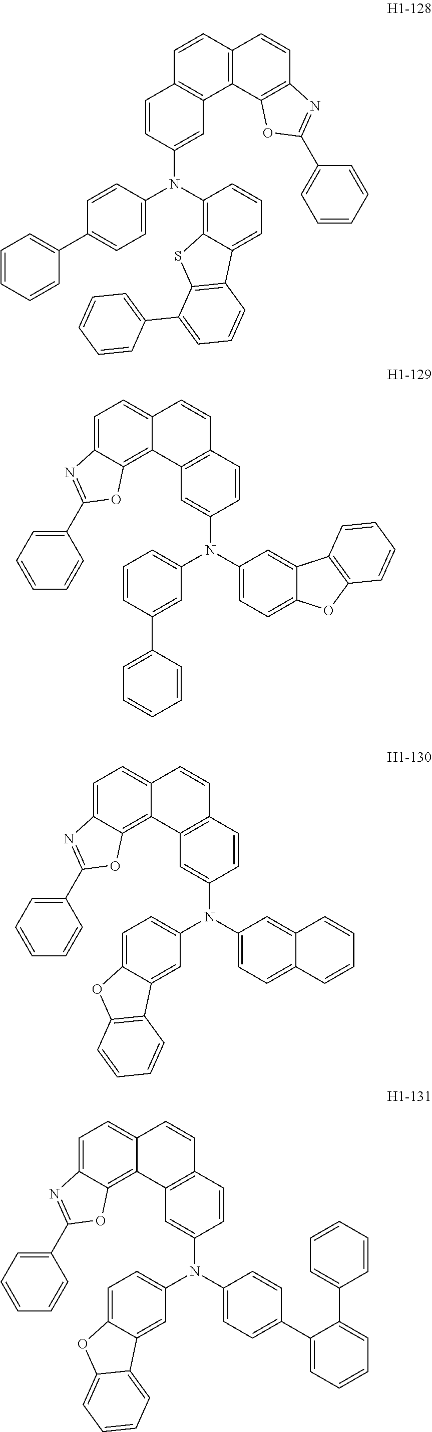 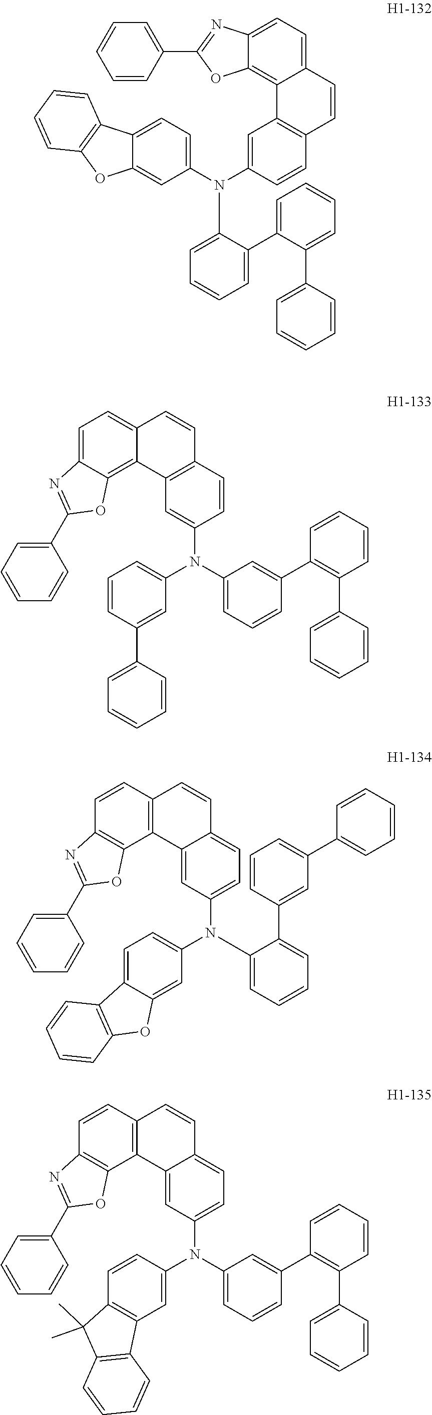 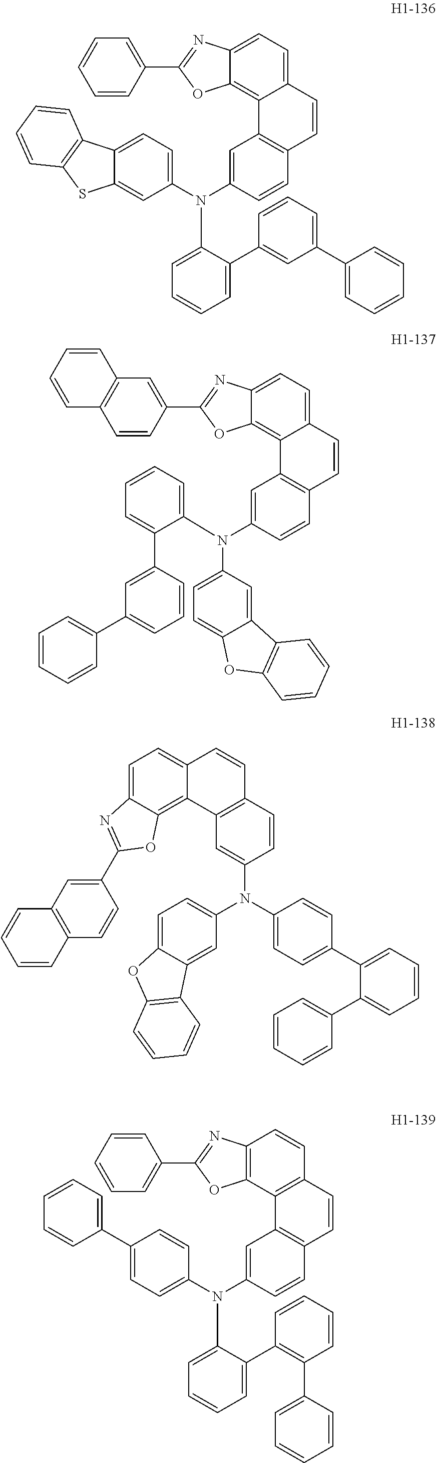 [SMILES: c1ccc(-c2cccc(-c3ccccc3N(c3ccc4c(c3)sc3ccccc34)c3ccc4ccc5ccc6nc(-c7ccccc7)oc6c5c4c3)c2)cc1 ; c1ccc(-c2cccc(-c3ccccc3N(c3ccc4oc5ccccc5c4c3)c3ccc4ccc5ccc6nc(-c7ccc8ccccc8c7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1] 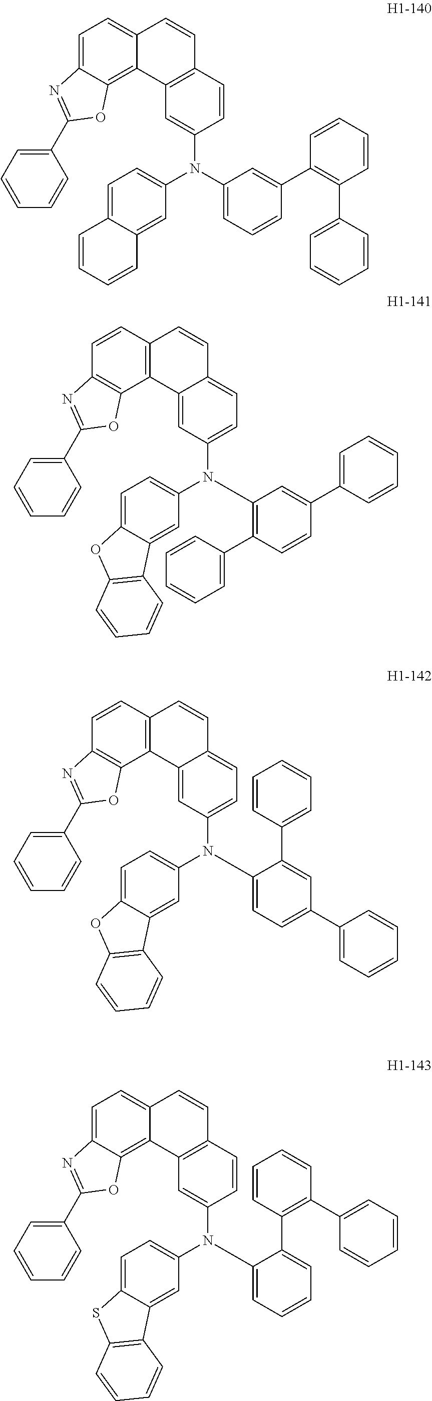 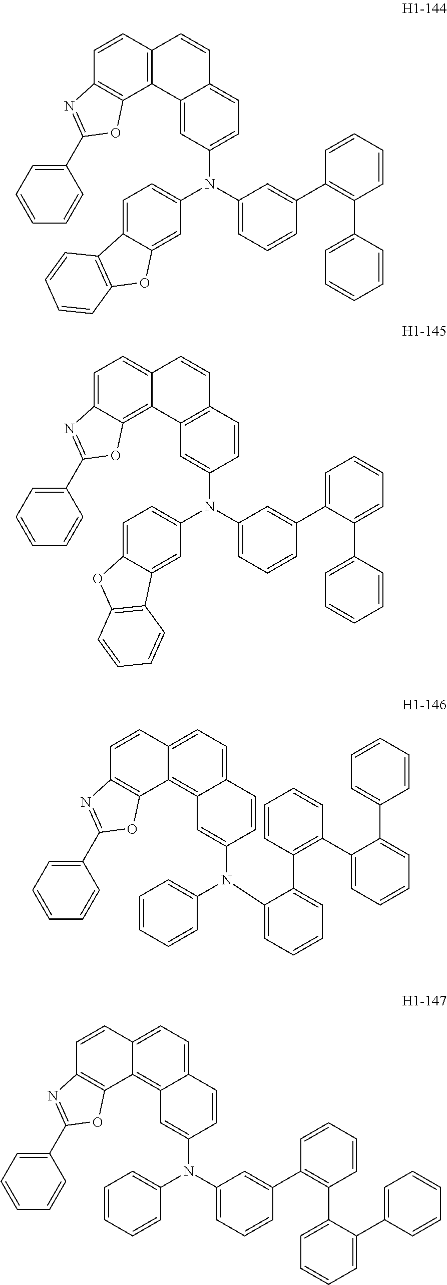 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1 ; c1ccc(-c2nc3ccc4ccc5ccc(N(c6ccccc6)c6ccccc6-c6ccccc6-c6ccccc6-c6ccccc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1] 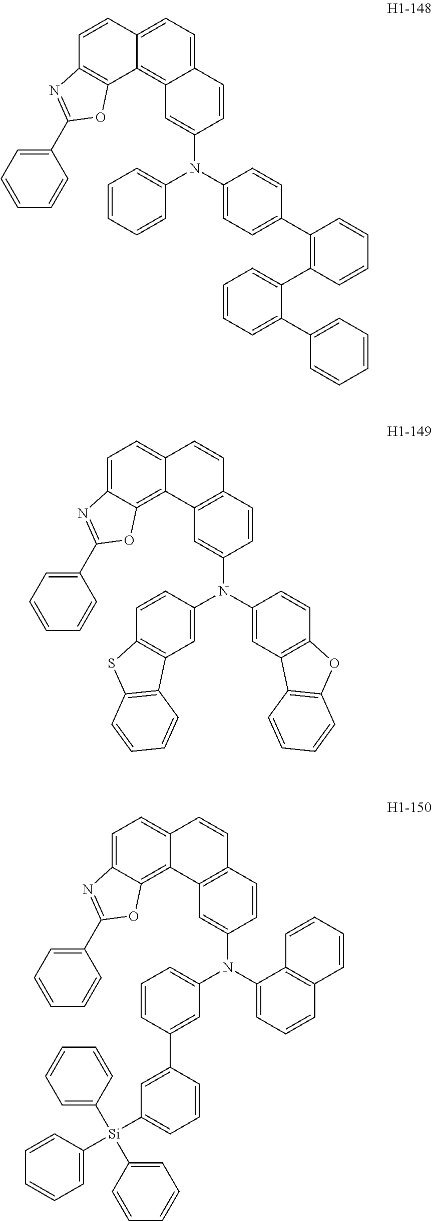 [SMILES: c1ccc(-c2nc3ccc4ccc5ccc(N(c6ccccc6)c6ccc(-c7ccccc7-c7ccccc7-c7ccccc7)cc6)cc5c4c3o2)cc1 ; c1ccc(-c2nc3ccc4ccc5ccc(N(c6ccc7oc8ccccc8c7c6)c6ccc7sc8ccccc8c7c6)cc5c4c3o2)cc1 ; c1ccc(-c2nc3ccc4ccc5ccc(N(c6cccc(-c7cccc([Si](c8ccccc8)(c8ccccc8)c8ccccc8)c7)c6)c6cccc7ccccc67)cc5c4c3o2)cc1] 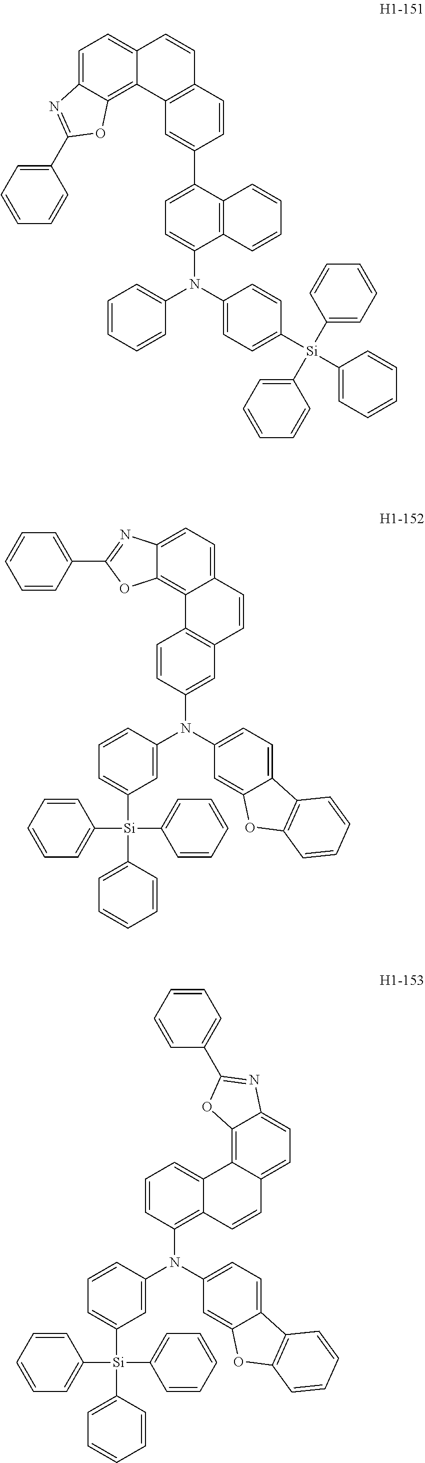 [SMILES: c1ccc(-c2nc3ccc4ccc5ccc(-c6ccc(N(c7ccccc7)c7ccc([Si](c8ccccc8)(c8ccccc8)c8ccccc8)cc7)c7ccccc67)cc5c4c3o2)cc1 ; c1ccc(-c2nc3ccc4ccc5cc(N(c6cccc([Si](c7ccccc7)(c7ccccc7)c7ccccc7)c6)c6ccc7c(c6)oc6ccccc67)ccc5c4c3o2)cc1 ; c1ccc(-c2nc3ccc4ccc5c(N(c6cccc([Si](c7ccccc7)(c7ccccc7)c7ccccc7)c6)c6ccc7c(c6)oc6ccccc67)cccc5c4c3o2)cc1] 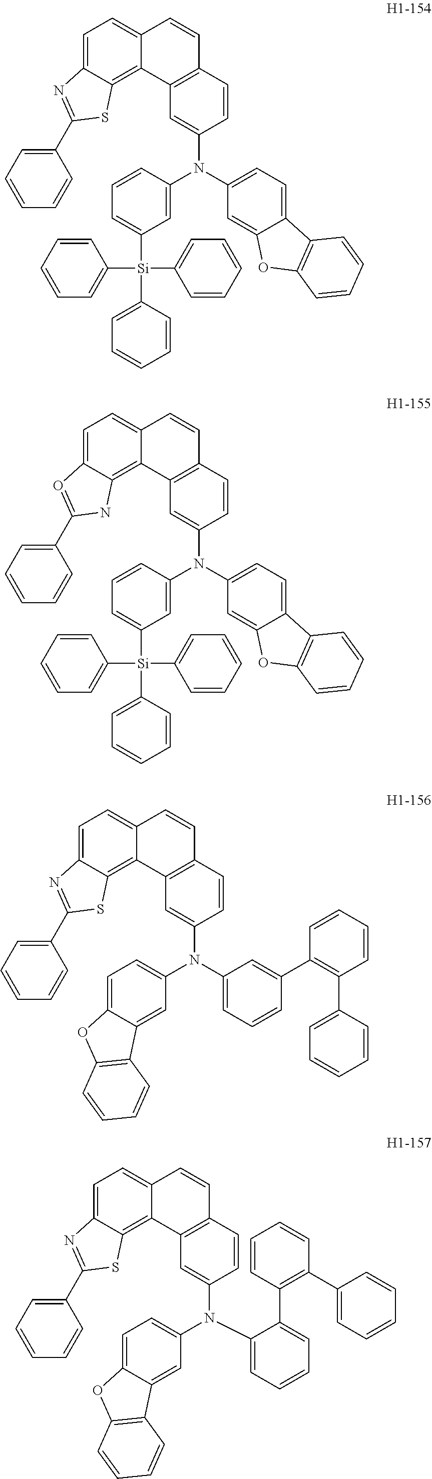 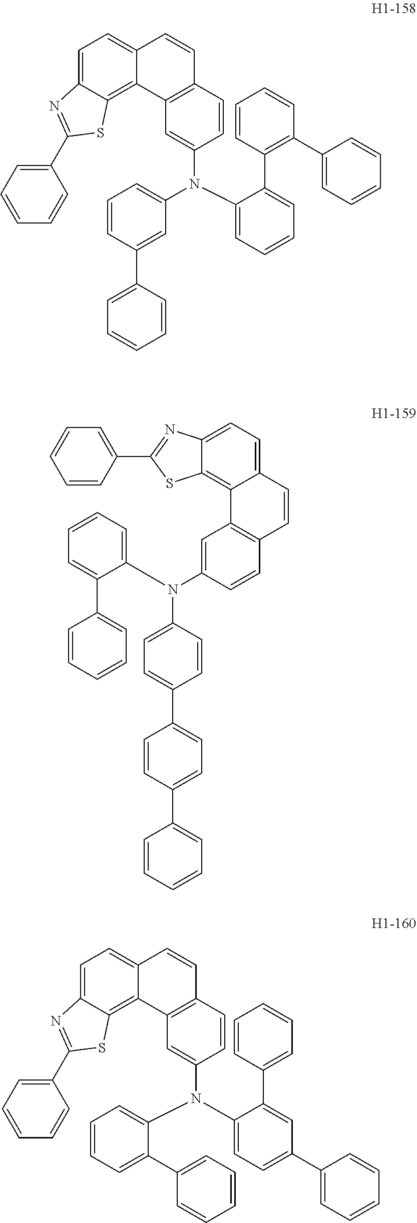 [SMILES: c1ccc(-c2cccc(N(c3ccc4ccc5ccc6nc(-c7ccccc7)sc6c5c4c3)c3ccccc3-c3ccccc3-c3ccccc3)c2)cc1 ; c1ccc(-c2ccc(-c3ccc(N(c4ccc5ccc6ccc7nc(-c8ccccc8)sc7c6c5c4)c4ccccc4-c4ccccc4)cc3)cc2)cc1 ; c1ccc(-c2ccc(N(c3ccc4ccc5ccc6nc(-c7ccccc7)sc6c5c4c3)c3ccccc3-c3ccccc3)c(-c3ccccc3)c2)cc1] 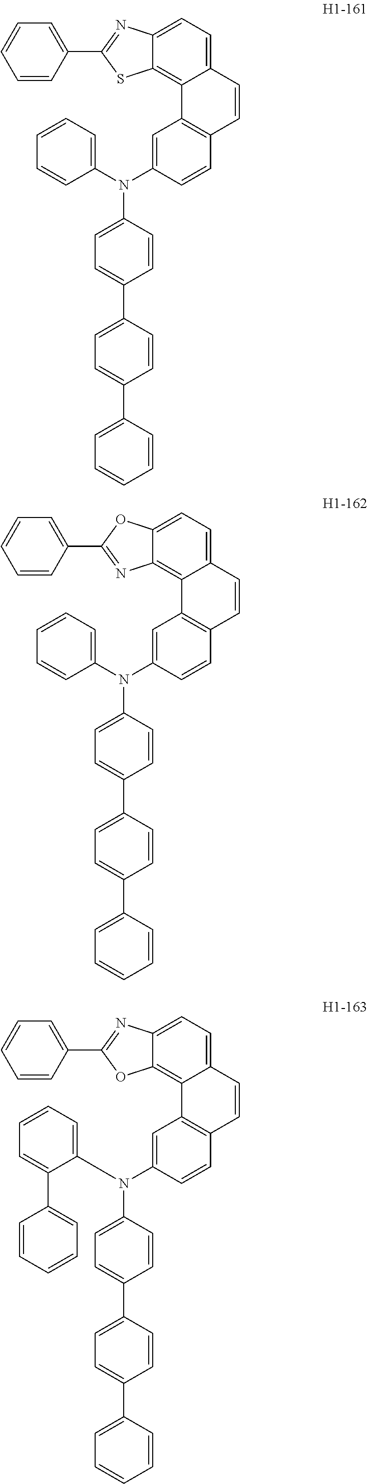 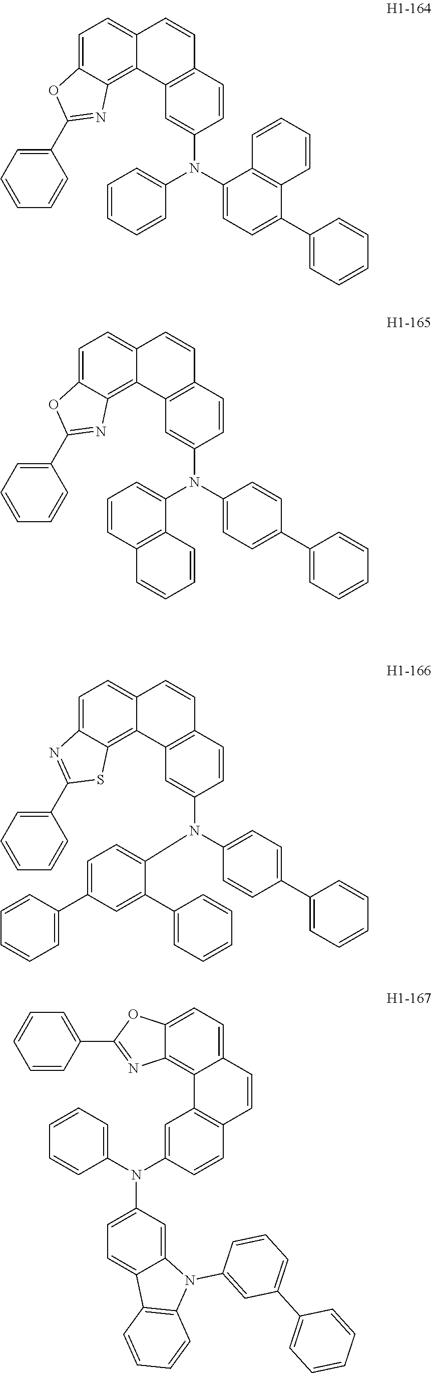 [SMILES: c1ccc(-c2nc3c(ccc4ccc5ccc(N(c6ccccc6)c6ccc(-c7ccccc7)c7ccccc67)cc5c43)o2)cc1 ; c1ccc(-c2ccc(N(c3ccc4ccc5ccc6oc(-c7ccccc7)nc6c5c4c3)c3cccc4ccccc34)cc2)cc1 ; c1ccc(-c2ccc(N(c3ccc4ccc5ccc6nc(-c7ccccc7)sc6c5c4c3)c3ccc(-c4ccccc4)cc3-c3ccccc3)cc2)cc1 ; c1ccc(-c2cccc(-n3c4ccccc4c4ccc(N(c5ccccc5)c5ccc6ccc7ccc8oc(-c9ccccc9)nc8c7c6c5)cc43)c2)cc1] 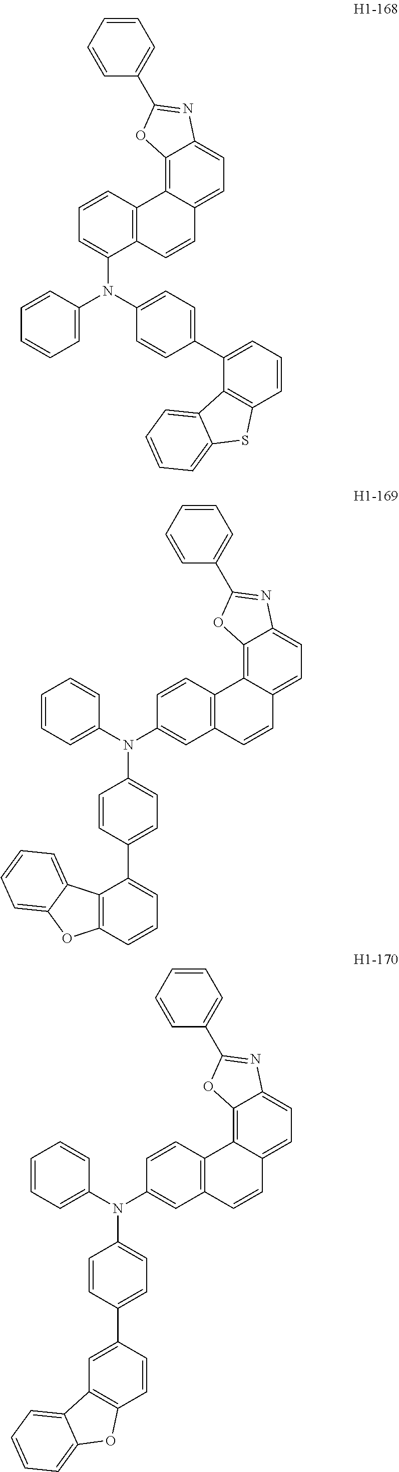 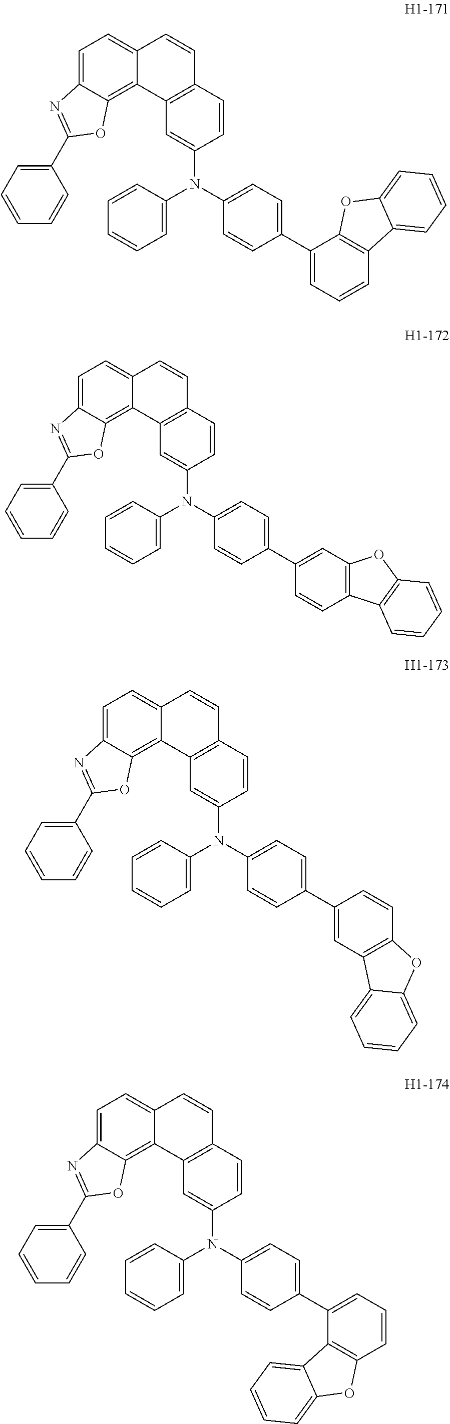 [SMILES: c1ccc(-c2nc3ccc4ccc5ccc(N(c6ccccc6)c6ccc(-c7cccc8c7oc7ccccc78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3o2)cc1 ; c1ccc(-c2nc3ccc4ccc5ccc(N(c6ccccc6)c6ccc(-c7ccc8oc9ccccc9c8c7)cc6)cc5c4c3o2)cc1 ; c1ccc(-c2nc3ccc4ccc5ccc(N(c6ccccc6)c6ccc(-c7cccc8oc9ccccc9c78)cc6)cc5c4c3o2)cc1] 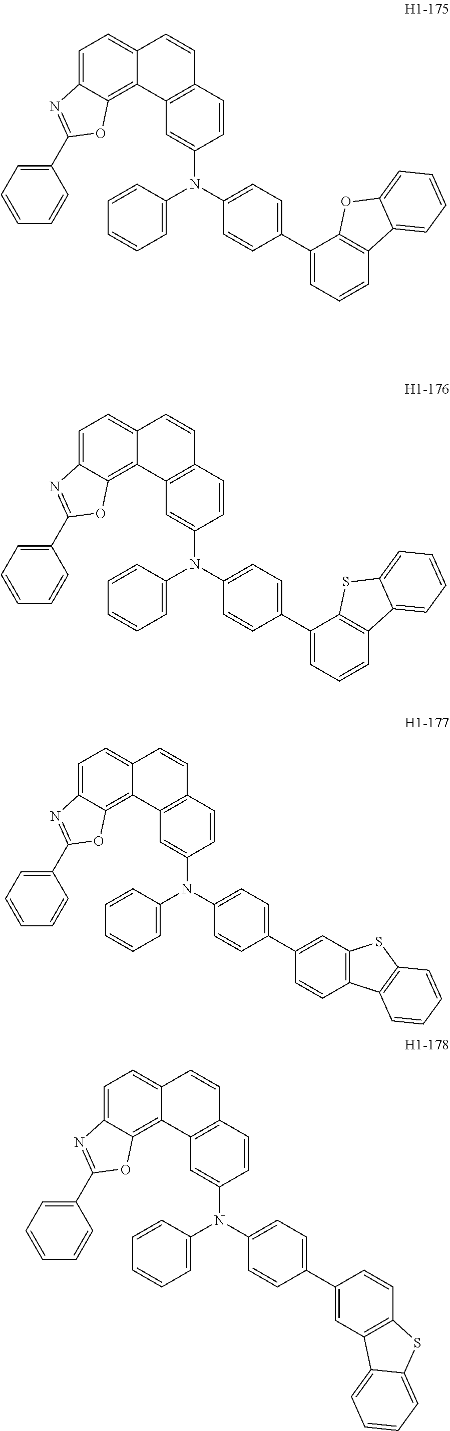 [SMILES: c1ccc(-c2nc3ccc4ccc5ccc(N(c6ccccc6)c6ccc(-c7cccc8c7oc7ccccc78)cc6)cc5c4c3o2)cc1 ; c1ccc(-c2nc3ccc4ccc5ccc(N(c6ccccc6)c6ccc(-c7cccc8c7sc7ccccc78)cc6)cc5c4c3o2)cc1 ; c1ccc(-c2nc3ccc4ccc5ccc(N(c6ccccc6)c6ccc(-c7ccc8c(c7)sc7ccccc78)cc6)cc5c4c3o2)cc1 ; c1ccc(-c2nc3ccc4ccc5ccc(N(c6ccccc6)c6ccc(-c7ccc8sc9ccccc9c8c7)cc6)cc5c4c3o2)cc1] 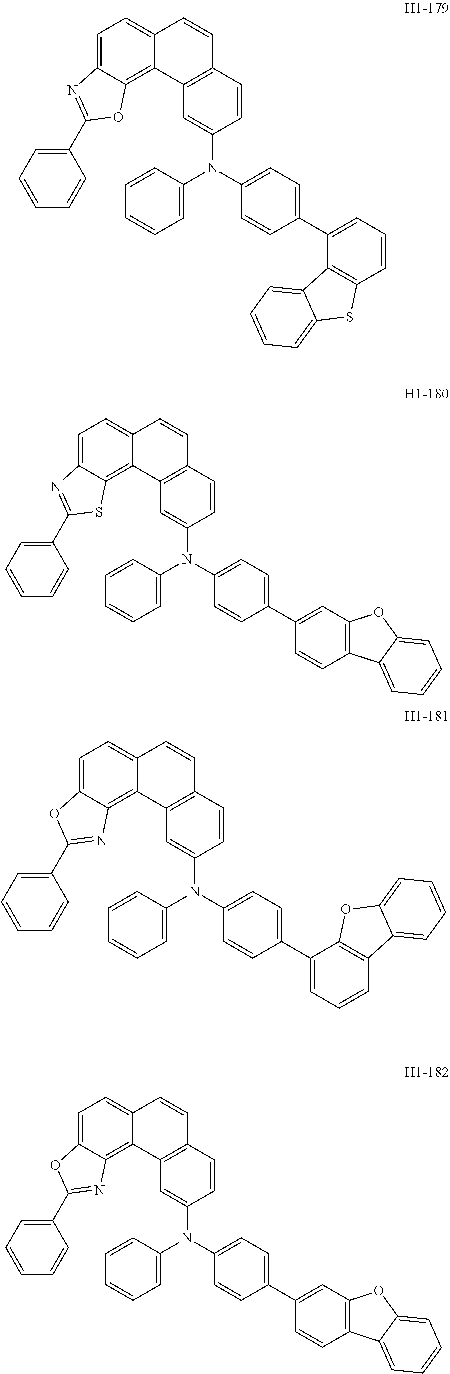 [SMILES: c1ccc(-c2nc3ccc4ccc5ccc(N(c6ccccc6)c6ccc(-c7cccc8sc9ccccc9c78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3s2)cc1 ; c1ccc(-c2nc3c(ccc4ccc5ccc(N(c6ccccc6)c6ccc(-c7cccc8c7oc7ccccc78)cc6)cc5c43)o2)cc1 ; c1ccc(-c2nc3c(ccc4ccc5ccc(N(c6ccccc6)c6ccc(-c7ccc8c(c7)oc7ccccc78)cc6)cc5c43)o2)cc1] 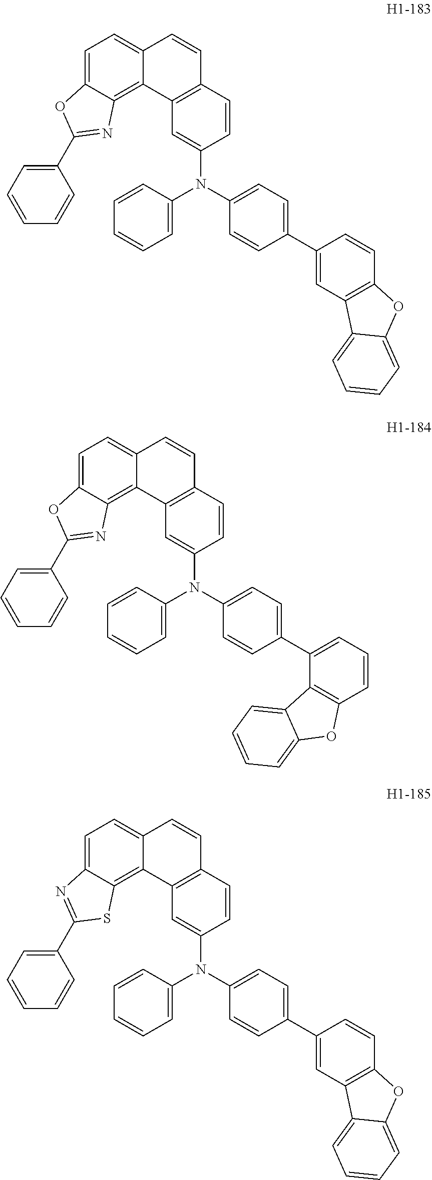 [SMILES: c1ccc(-c2nc3c(ccc4ccc5ccc(N(c6ccccc6)c6ccc(-c7ccc8oc9ccccc9c8c7)cc6)cc5c43)o2)cc1 ; c1ccc(-c2nc3c(ccc4ccc5ccc(N(c6ccccc6)c6ccc(-c7cccc8oc9ccccc9c78)cc6)cc5c43)o2)cc1 ; c1ccc(-c2nc3ccc4ccc5ccc(N(c6ccccc6)c6ccc(-c7ccc8oc9ccccc9c8c7)cc6)cc5c4c3s2)cc1] 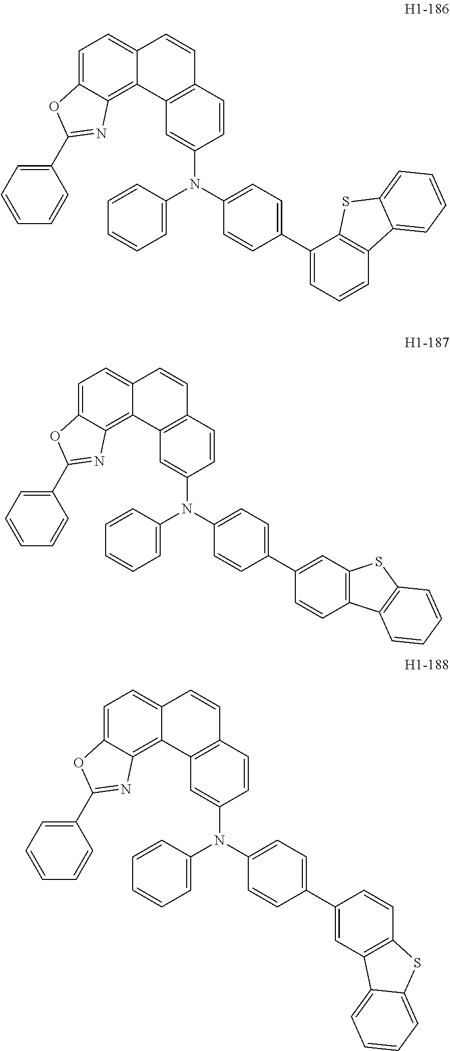 [SMILES: c1ccc(-c2nc3c(ccc4ccc5ccc(N(c6ccccc6)c6ccc(-c7cccc8c7sc7ccccc78)cc6)cc5c43)o2)cc1 ; c1ccc(-c2nc3c(ccc4ccc5ccc(N(c6ccccc6)c6ccc(-c7ccc8c(c7)sc7ccccc78)cc6)cc5c43)o2)cc1 ; c1ccc(-c2nc3c(ccc4ccc5ccc(N(c6ccccc6)c6ccc(-c7ccc8sc9ccccc9c8c7)cc6)cc5c43)o2)cc1] 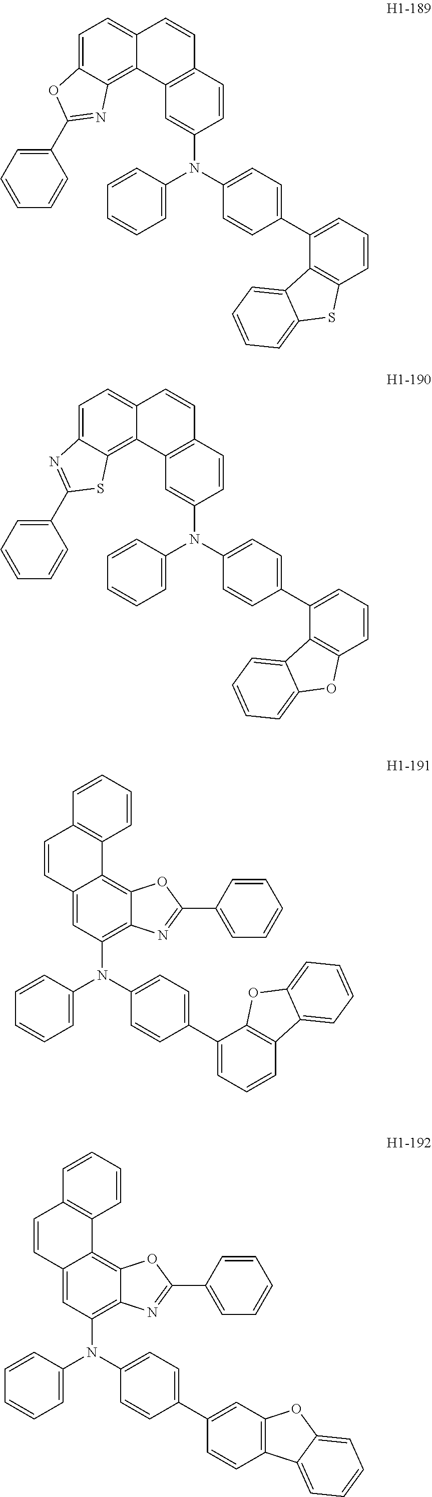 [SMILES: c1ccc(-c2nc3c(ccc4ccc5ccc(N(c6ccccc6)c6ccc(-c7cccc8sc9ccccc9c78)cc6)cc5c43)o2)cc1 ; c1ccc(-c2nc3ccc4ccc5ccc(N(c6ccccc6)c6ccc(-c7cccc8oc9ccccc9c78)cc6)cc5c4c3s2)cc1 ; c1ccc(-c2nc3c(N(c4ccccc4)c4ccc(-c5cccc6c5oc5ccccc56)cc4)cc4ccc5ccccc5c4c3o2)cc1 ; c1ccc(-c2nc3c(N(c4ccccc4)c4ccc(-c5ccc6c(c5)oc5ccccc56)cc4)cc4ccc5ccccc5c4c3o2)cc1] 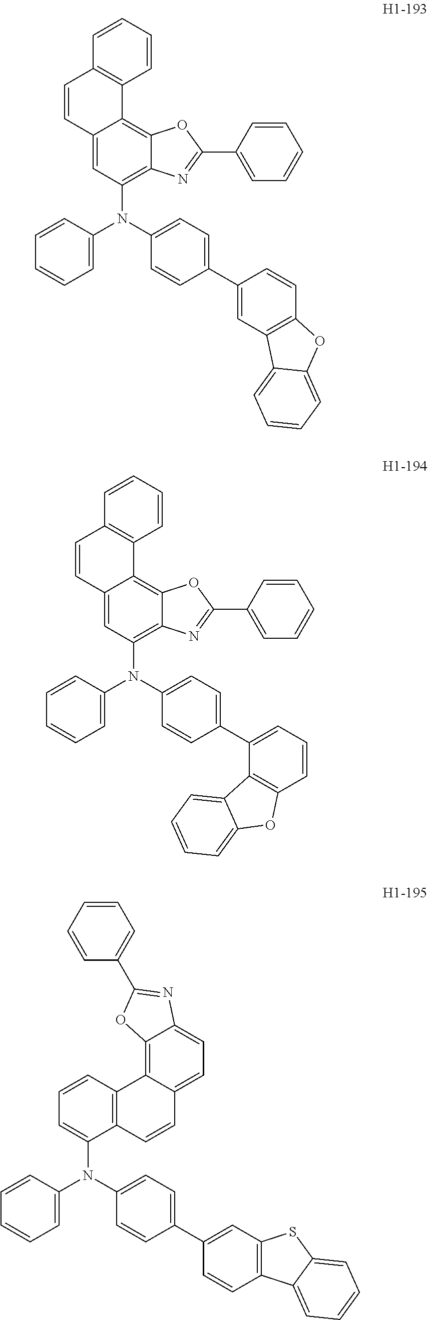 [SMILES: c1ccc(-c2nc3c(N(c4ccccc4)c4ccc(-c5ccc6oc7ccccc7c6c5)cc4)cc4ccc5ccccc5c4c3o2)cc1 ; c1ccc(-c2nc3c(N(c4ccccc4)c4ccc(-c5cccc6oc7ccccc7c56)cc4)cc4ccc5ccccc5c4c3o2)cc1 ; c1ccc(-c2nc3ccc4ccc5c(N(c6ccccc6)c6ccc(-c7ccc8c(c7)sc7ccccc78)cc6)cccc5c4c3o2)cc1] 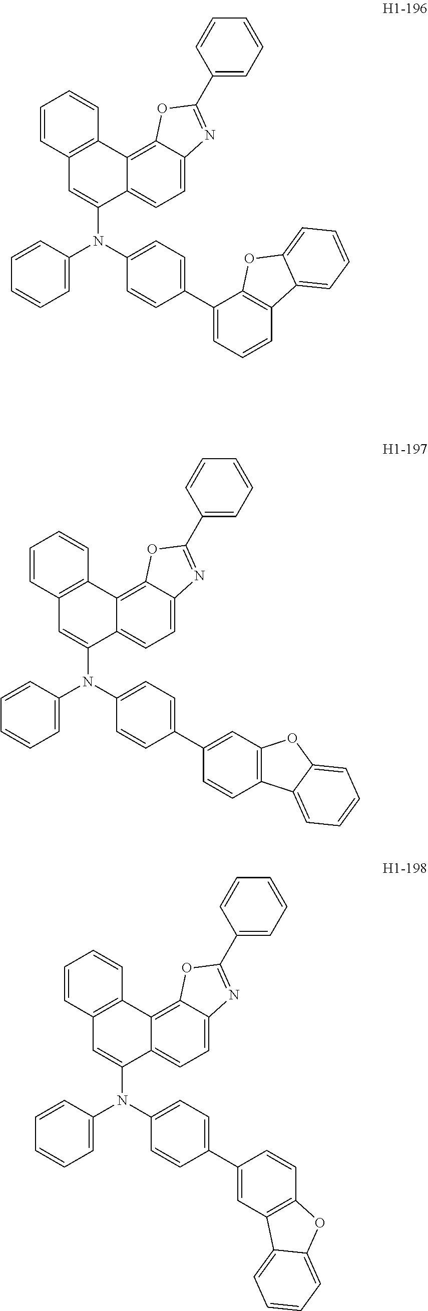 [SMILES: c1ccc(-c2nc3ccc4c(N(c5ccccc5)c5ccc(-c6cccc7c6oc6ccccc67)cc5)cc5ccccc5c4c3o2)cc1 ; c1ccc(-c2nc3ccc4c(N(c5ccccc5)c5ccc(-c6ccc7c(c6)oc6ccccc67)cc5)cc5ccccc5c4c3o2)cc1 ; c1ccc(-c2nc3ccc4c(N(c5ccccc5)c5ccc(-c6ccc7oc8ccccc8c7c6)cc5)cc5ccccc5c4c3o2)cc1] 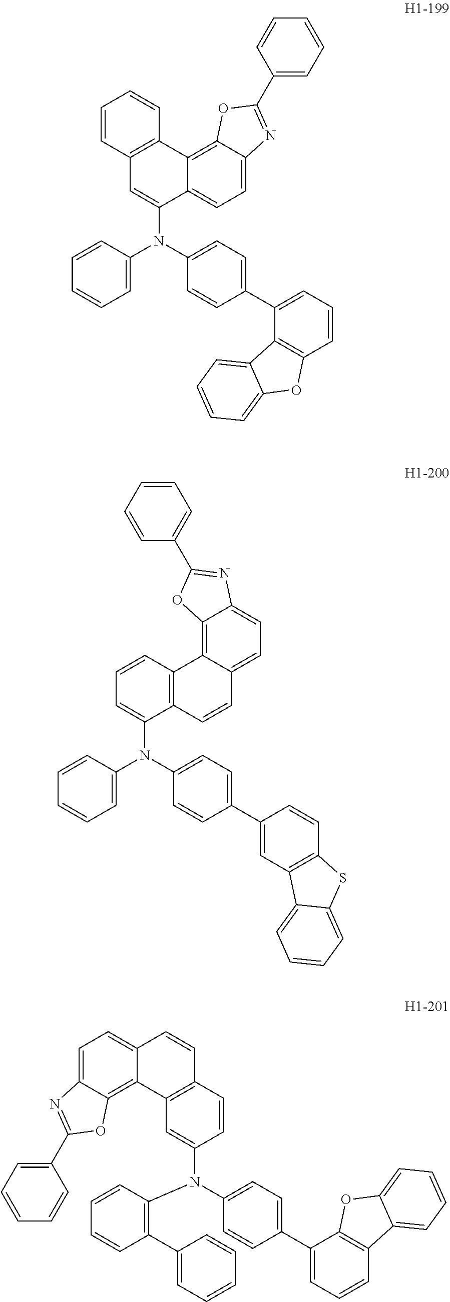 [SMILES: c1ccc(-c2nc3ccc4c(N(c5ccccc5)c5ccc(-c6cccc7oc8ccccc8c67)cc5)cc5ccccc5c4c3o2)cc1 ; c1ccc(-c2nc3ccc4ccc5c(N(c6ccccc6)c6ccc(-c7ccc8sc9ccccc9c8c7)cc6)cccc5c4c3o2)cc1 ; c1ccc(-c2nc3ccc4ccc5ccc(N(c6ccc(-c7cccc8c7oc7ccccc78)cc6)c6ccccc6-c6ccccc6)cc5c4c3o2)cc1] 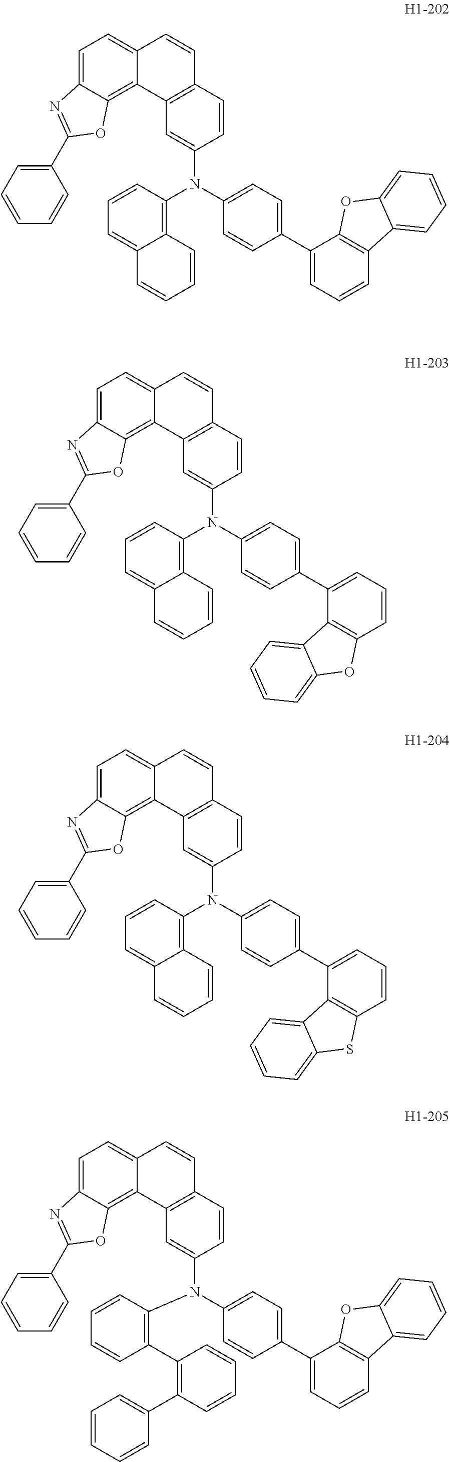 [SMILES: c1ccc(-c2nc3ccc4ccc5ccc(N(c6ccc(-c7cccc8c7oc7ccccc78)cc6)c6cccc7ccccc67)cc5c4c3o2)cc1 ; c1ccc(-c2nc3ccc4ccc5ccc(N(c6ccc(-c7cccc8oc9ccccc9c78)cc6)c6cccc7ccccc67)cc5c4c3o2)cc1 ; c1ccc(-c2nc3ccc4ccc5ccc(N(c6ccc(-c7cccc8sc9ccccc9c78)cc6)c6cccc7ccccc67)cc5c4c3o2)cc1 ; c1ccc(-c2nc3ccc4ccc5ccc(N(c6ccc(-c7cccc8c7oc7ccccc78)cc6)c6ccccc6-c6ccccc6-c6ccccc6)cc5c4c3o2)cc1] 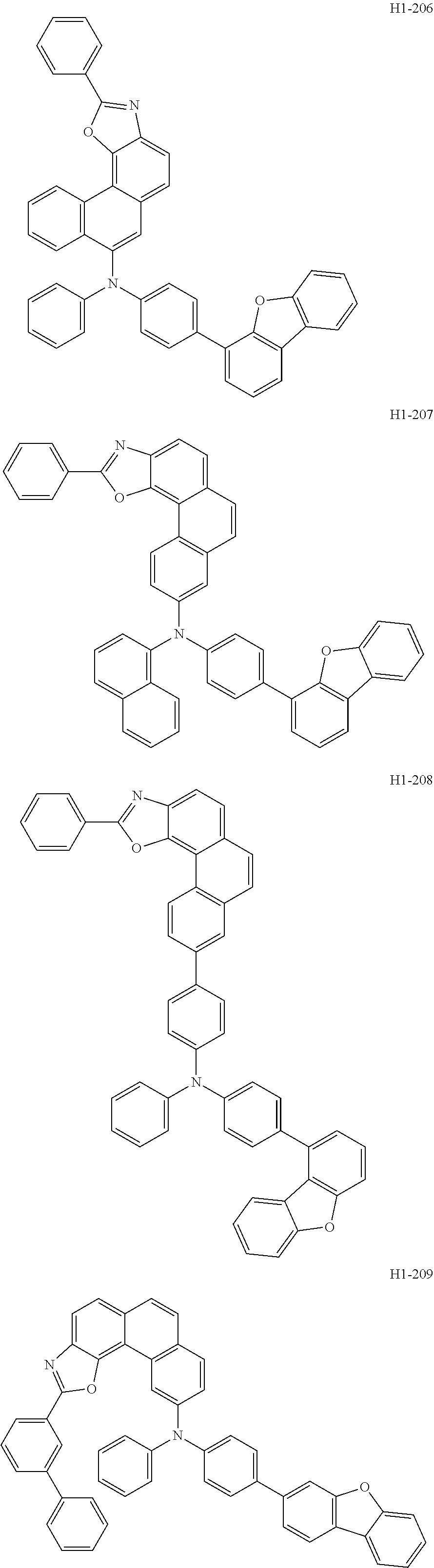 [SMILES: c1ccc(-c2nc3ccc4cc(N(c5ccccc5)c5ccc(-c6cccc7c6oc6ccccc67)cc5)c5ccccc5c4c3o2)cc1 ; c1ccc(-c2nc3ccc4ccc5cc(N(c6ccc(-c7cccc8c7oc7ccccc78)cc6)c6cccc7ccccc67)ccc5c4c3o2)cc1 ; c1ccc(-c2nc3ccc4ccc5cc(-c6ccc(N(c7ccccc7)c7ccc(-c8cccc9oc%10ccccc%10c89)cc7)cc6)ccc5c4c3o2)cc1 ; c1ccc(-c2cccc(-c3nc4ccc5ccc6ccc(N(c7ccccc7)c7ccc(-c8ccc9c(c8)oc8ccccc89)cc7)cc6c5c4o3)c2)cc1] 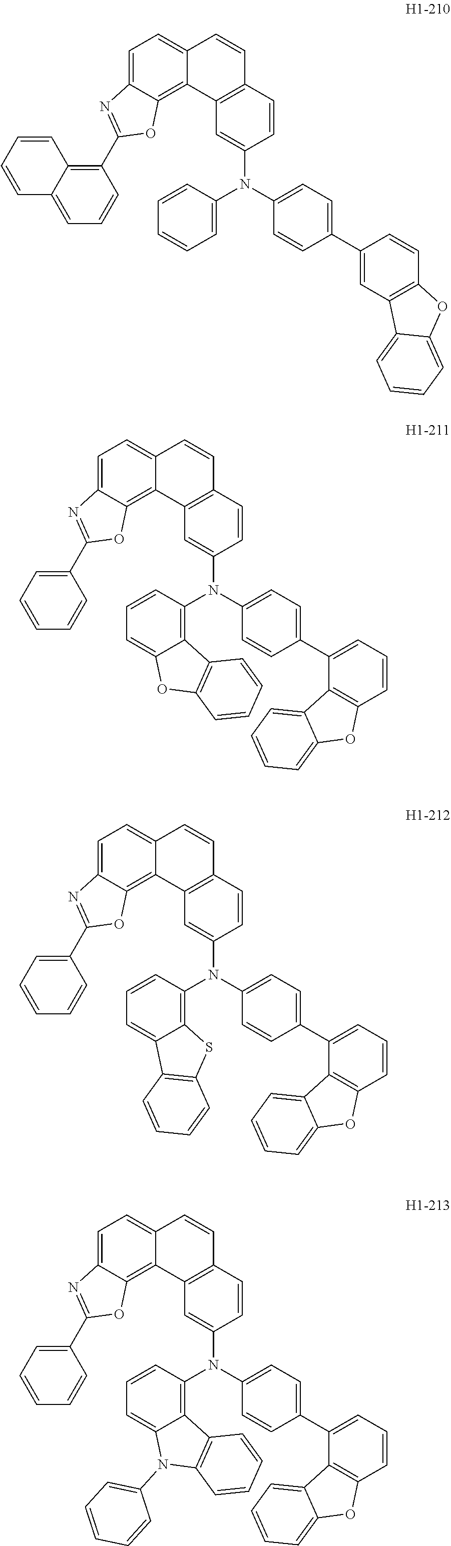 [SMILES: c1ccc(N(c2ccc(-c3ccc4oc5ccccc5c4c3)cc2)c2ccc3ccc4ccc5nc(-c6cccc7ccccc67)oc5c4c3c2)cc1 ; c1ccc(-c2nc3ccc4ccc5ccc(N(c6ccc(-c7cccc8oc9ccccc9c78)cc6)c6cccc7oc8ccccc8c67)cc5c4c3o2)cc1 ; c1ccc(-c2nc3ccc4ccc5ccc(N(c6ccc(-c7cccc8oc9ccccc9c78)cc6)c6cccc7c6sc6ccccc67)cc5c4c3o2)cc1 ; c1ccc(-c2nc3ccc4ccc5ccc(N(c6ccc(-c7cccc8oc9ccccc9c78)cc6)c6cccc7c6c6ccccc6n7-c6ccccc6)cc5c4c3o2)cc1] 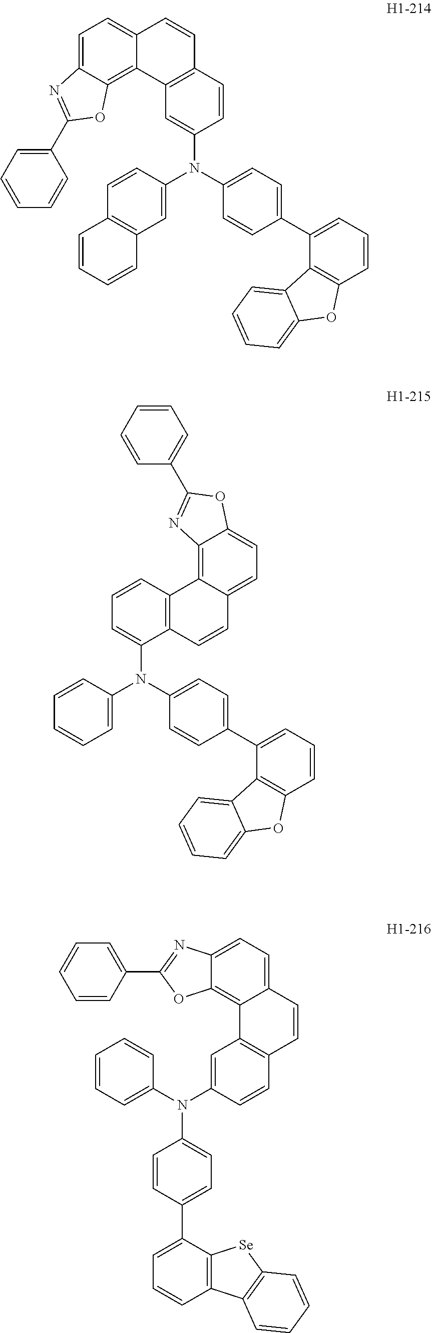 [SMILES: c1ccc(-c2nc3ccc4ccc5ccc(N(c6ccc(-c7cccc8oc9ccccc9c78)cc6)c6ccc7ccccc7c6)cc5c4c3o2)cc1 ; c1ccc(-c2nc3c(ccc4ccc5c(N(c6ccccc6)c6ccc(-c7cccc8oc9ccccc9c78)cc6)cccc5c43)o2)cc1 ; c1ccc(-c2nc3ccc4ccc5ccc(N(c6ccccc6)c6ccc(-c7cccc8c7[se]c7ccccc78)cc6)cc5c4c3o2)cc1] 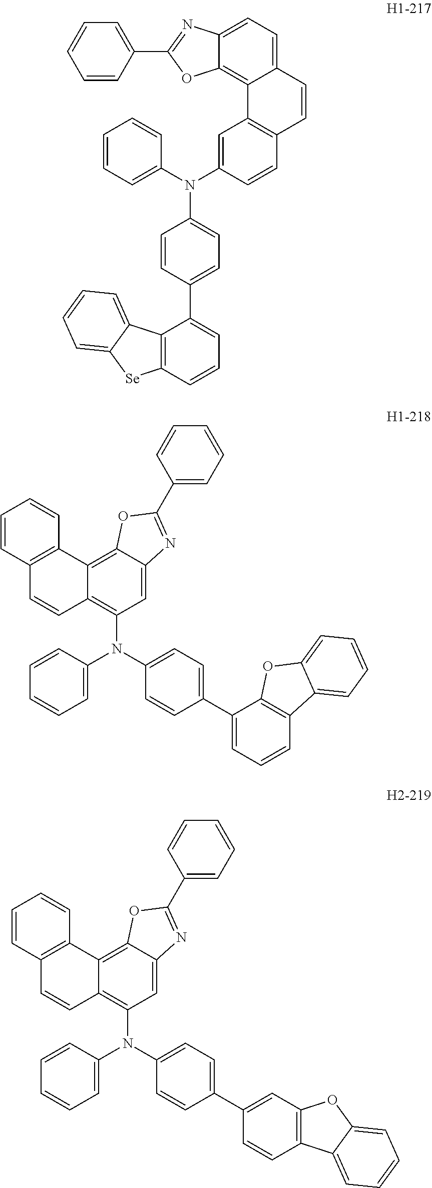 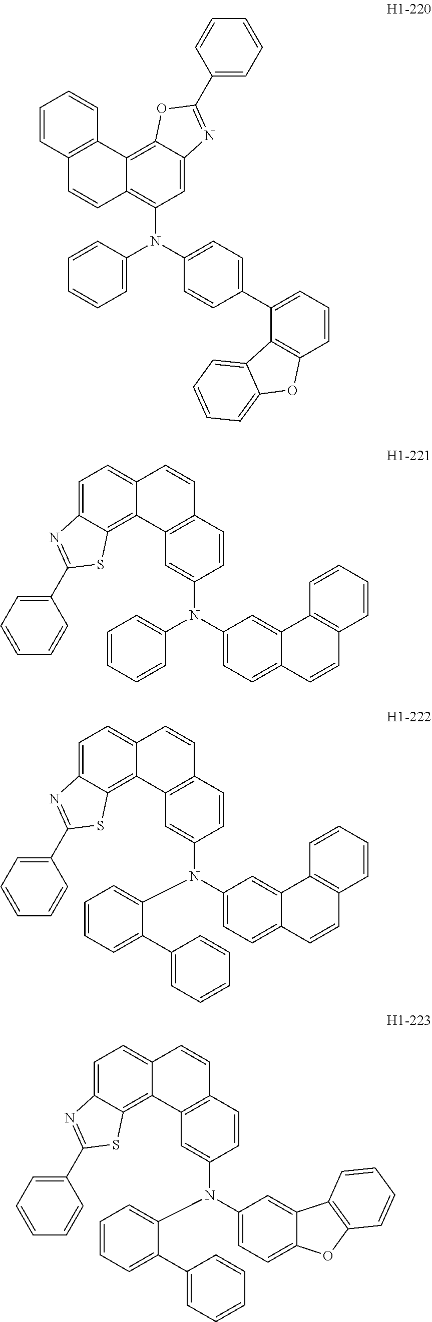 [SMILES: c1ccc(-c2nc3cc(N(c4ccccc4)c4ccc(-c5cccc6oc7ccccc7c56)cc4)c4ccc5ccccc5c4c3o2)cc1 ; c1ccc(-c2nc3ccc4ccc5ccc(N(c6ccccc6)c6ccc7ccc8ccccc8c7c6)cc5c4c3s2)cc1 ; c1ccc(-c2nc3ccc4ccc5ccc(N(c6ccc7ccc8ccccc8c7c6)c6ccccc6-c6ccccc6)cc5c4c3s2)cc1 ; c1ccc(-c2nc3ccc4ccc5ccc(N(c6ccc7oc8ccccc8c7c6)c6ccccc6-c6ccccc6)cc5c4c3s2)cc1] 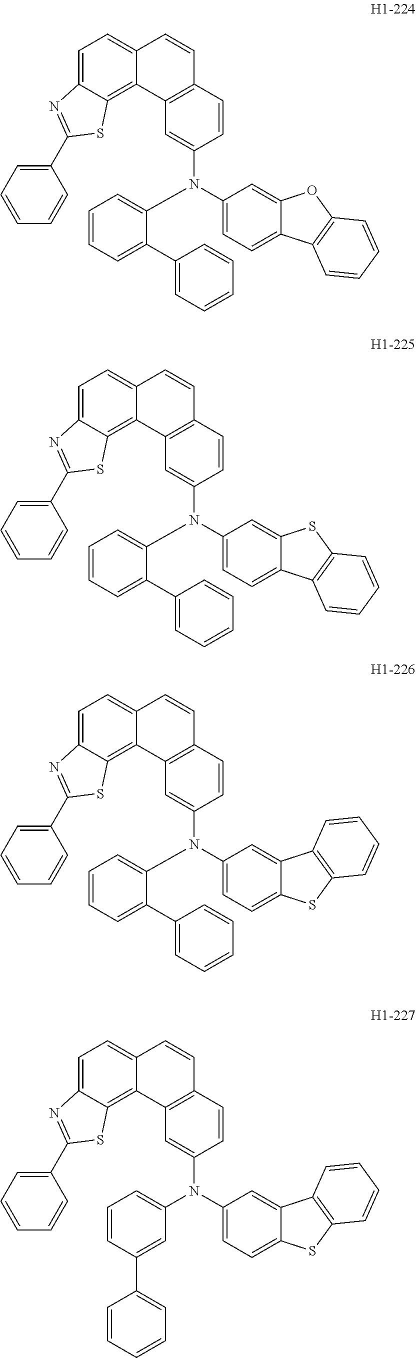 [SMILES: c1ccc(-c2nc3ccc4ccc5ccc(N(c6ccc7c(c6)oc6ccccc67)c6ccccc6-c6ccccc6)cc5c4c3s2)cc1 ; c1ccc(-c2nc3ccc4ccc5ccc(N(c6ccc7c(c6)sc6ccccc67)c6ccccc6-c6ccccc6)cc5c4c3s2)cc1 ; c1ccc(-c2nc3ccc4ccc5ccc(N(c6ccc7sc8ccccc8c7c6)c6ccccc6-c6ccccc6)cc5c4c3s2)cc1 ; c1ccc(-c2cccc(N(c3ccc4sc5ccccc5c4c3)c3ccc4ccc5ccc6nc(-c7ccccc7)sc6c5c4c3)c2)cc1] 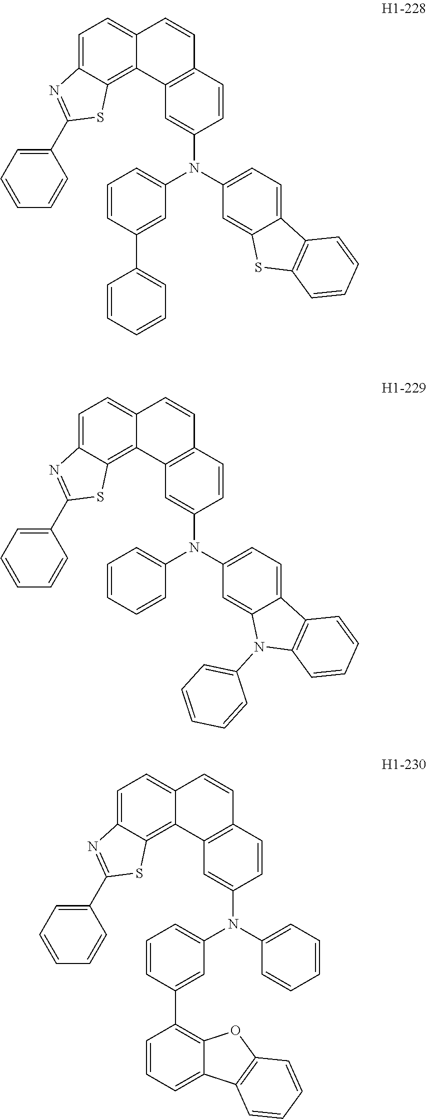 [SMILES: c1ccc(-c2cccc(N(c3ccc4c(c3)sc3ccccc34)c3ccc4ccc5ccc6nc(-c7ccccc7)sc6c5c4c3)c2)cc1 ; c1ccc(-c2nc3ccc4ccc5ccc(N(c6ccccc6)c6ccc7c8ccccc8n(-c8ccccc8)c7c6)cc5c4c3s2)cc1 ; c1ccc(-c2nc3ccc4ccc5ccc(N(c6ccccc6)c6cccc(-c7cccc8c7oc7ccccc78)c6)cc5c4c3s2)cc1] 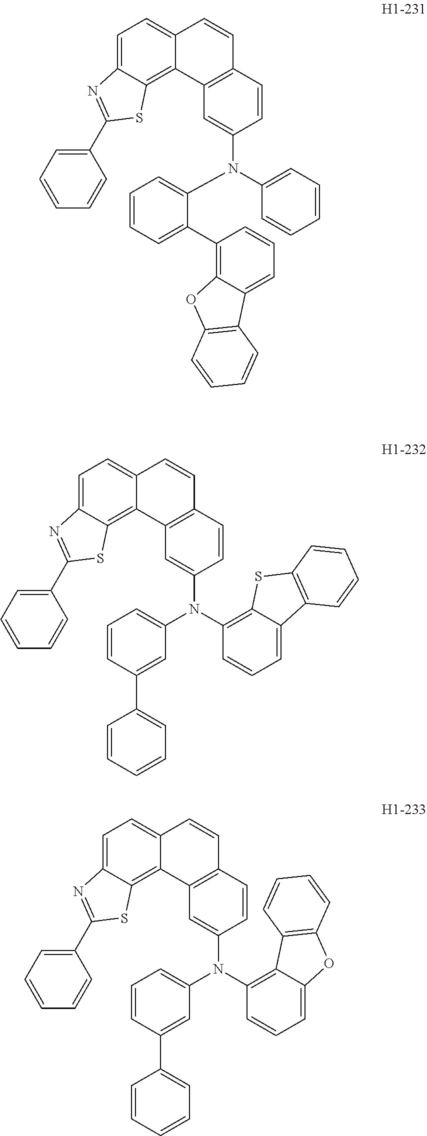 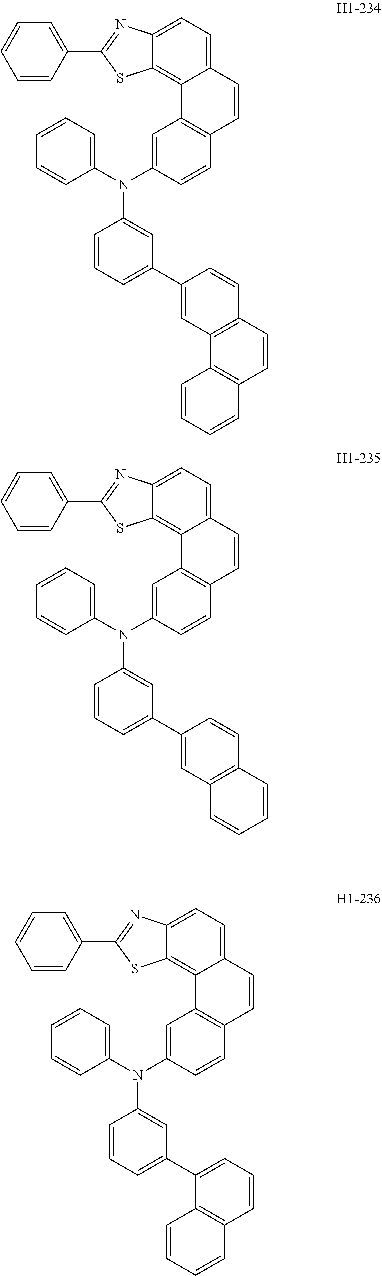 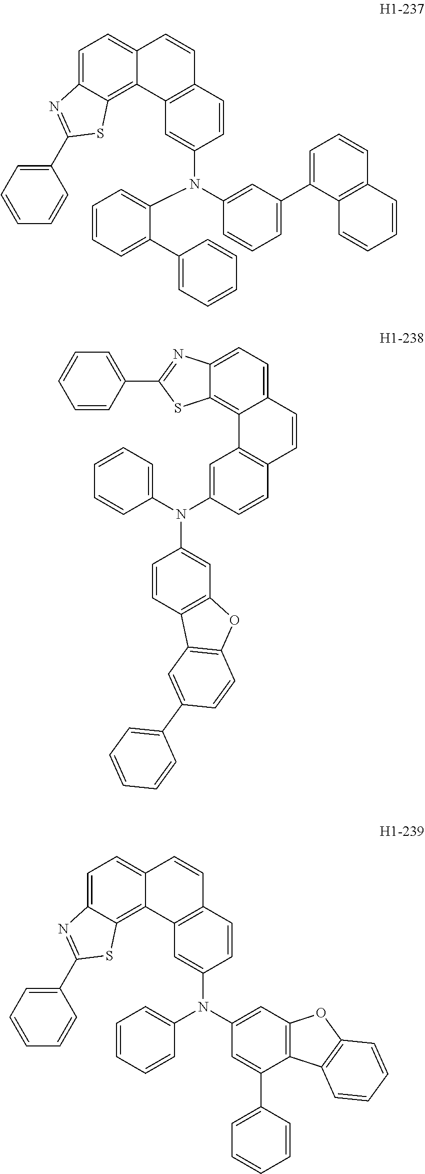 [SMILES: c1ccc(-c2nc3ccc4ccc5ccc(N(c6cccc(-c7cccc8ccccc78)c6)c6ccccc6-c6ccccc6)cc5c4c3s2)cc1 ; c1ccc(-c2ccc3oc4cc(N(c5ccccc5)c5ccc6ccc7ccc8nc(-c9ccccc9)sc8c7c6c5)ccc4c3c2)cc1 ; c1ccc(-c2nc3ccc4ccc5ccc(N(c6ccccc6)c6cc(-c7ccccc7)c7c(c6)oc6ccccc67)cc5c4c3s2)cc1] 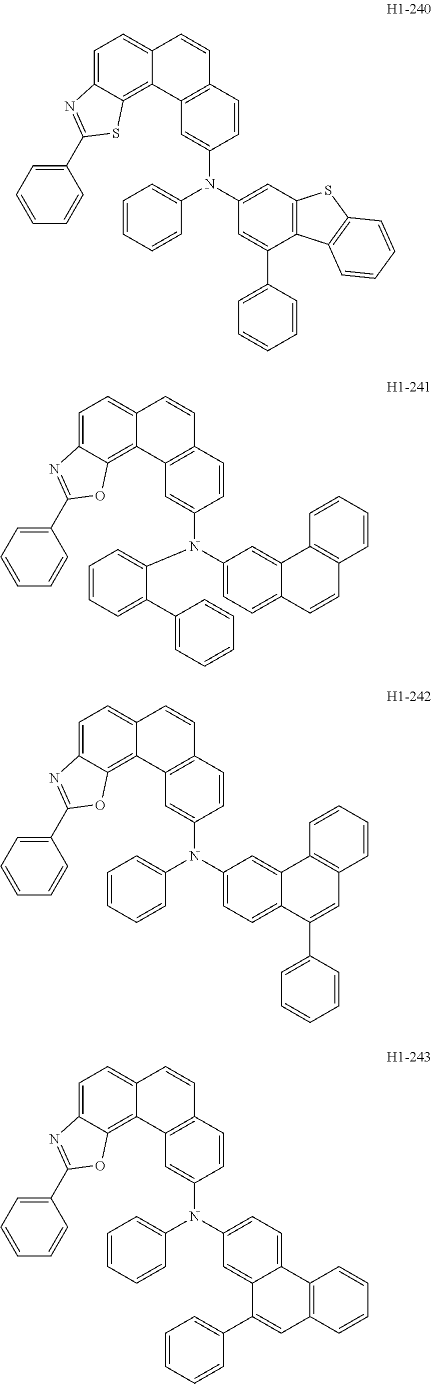 [SMILES: c1ccc(-c2nc3ccc4ccc5ccc(N(c6ccccc6)c6cc(-c7ccccc7)c7c(c6)sc6ccccc67)cc5c4c3s2)cc1 ; c1ccc(-c2nc3ccc4ccc5ccc(N(c6ccc7ccc8ccccc8c7c6)c6ccccc6-c6ccccc6)cc5c4c3o2)cc1 ; c1ccc(-c2nc3ccc4ccc5ccc(N(c6ccccc6)c6ccc7c(-c8ccccc8)cc8ccccc8c7c6)cc5c4c3o2)cc1 ; c1ccc(-c2nc3ccc4ccc5ccc(N(c6ccccc6)c6ccc7c(c6)c(-c6ccccc6)cc6ccccc67)cc5c4c3o2)cc1] 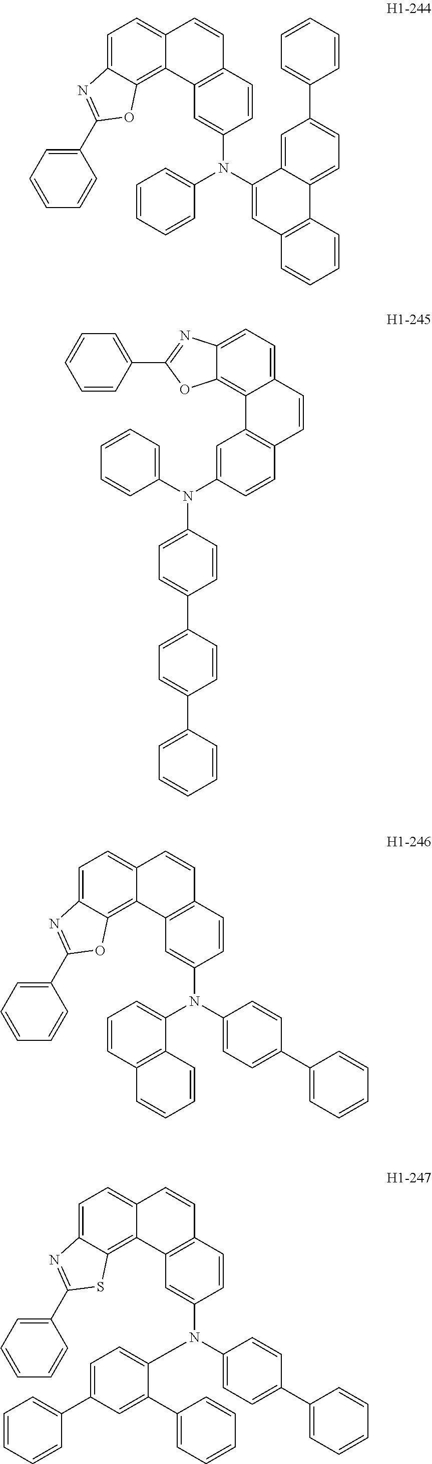 [SMILES: c1ccc(-c2ccc3c(c2)c(N(c2ccccc2)c2ccc4ccc5ccc6nc(-c7ccccc7)oc6c5c4c2)cc2ccccc23)cc1 ; c1ccc(-c2ccc(-c3ccc(N(c4ccccc4)c4ccc5ccc6ccc7nc(-c8ccccc8)oc7c6c5c4)cc3)cc2)cc1 ; c1ccc(-c2ccc(N(c3ccc4ccc5ccc6nc(-c7ccccc7)oc6c5c4c3)c3cccc4ccccc34)cc2)cc1 ; c1ccc(-c2ccc(N(c3ccc4ccc5ccc6nc(-c7ccccc7)sc6c5c4c3)c3ccc(-c4ccccc4)cc3-c3ccccc3)cc2)cc1] 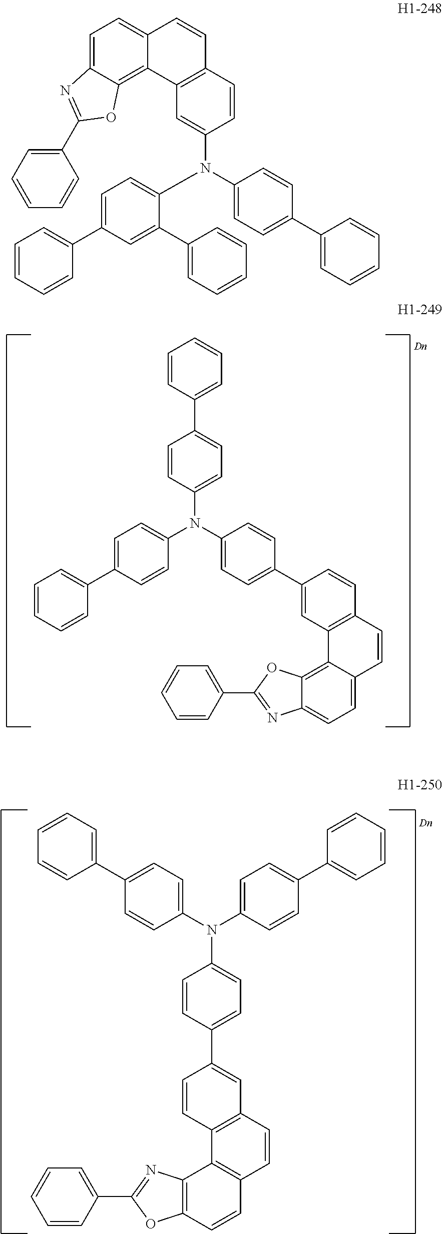 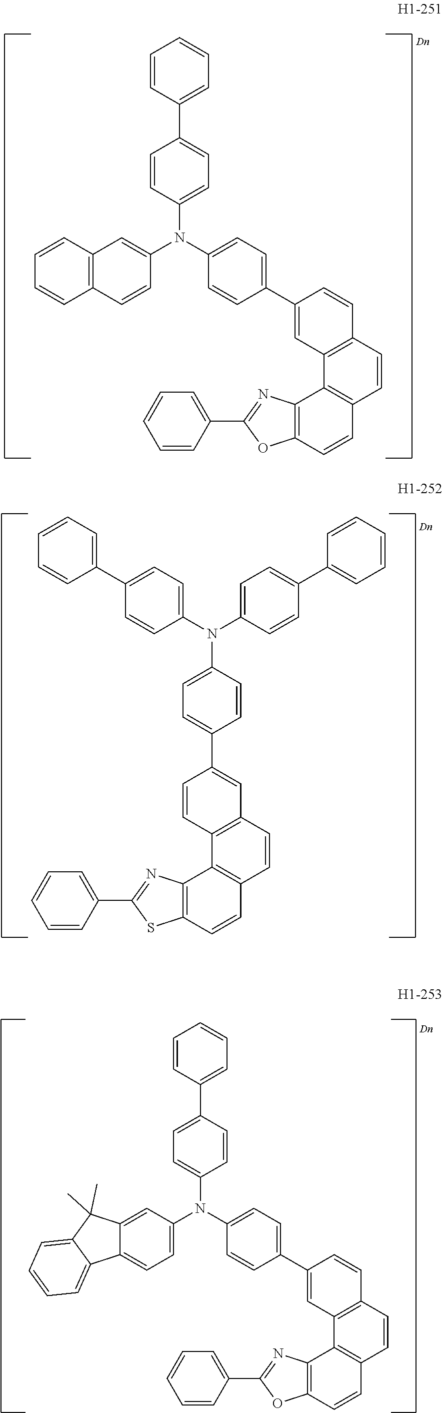 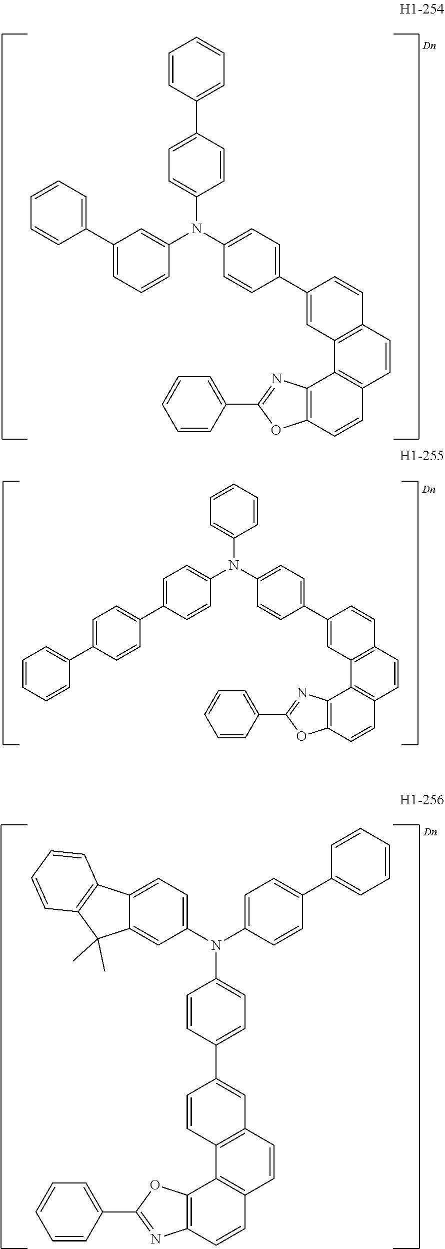 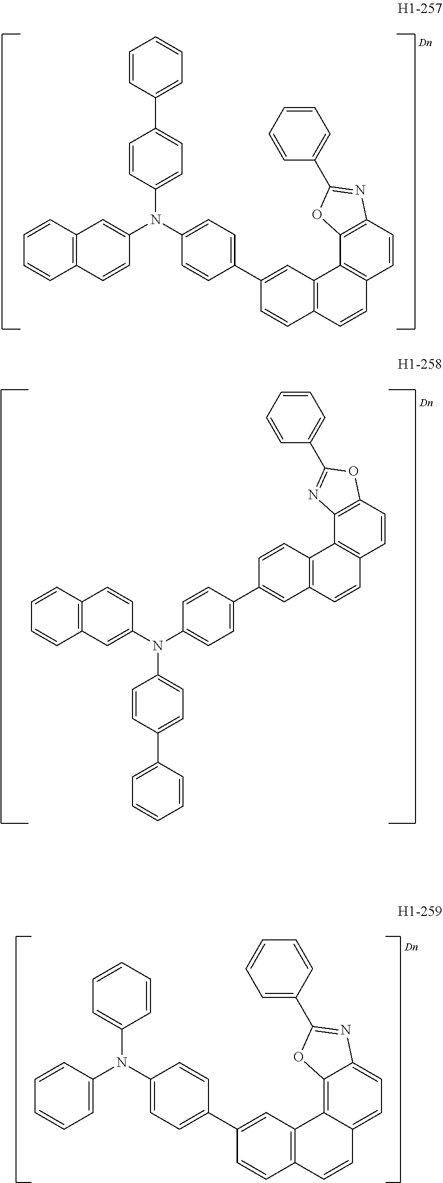 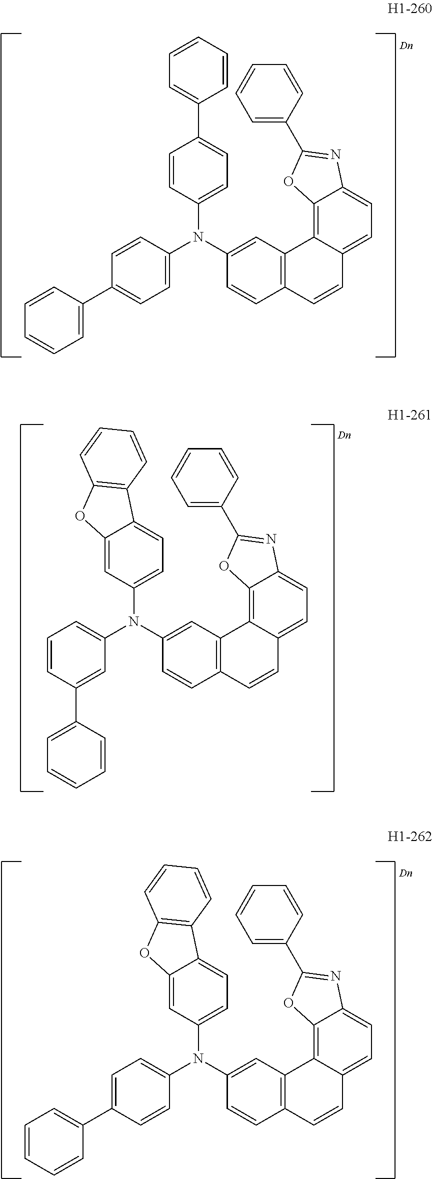 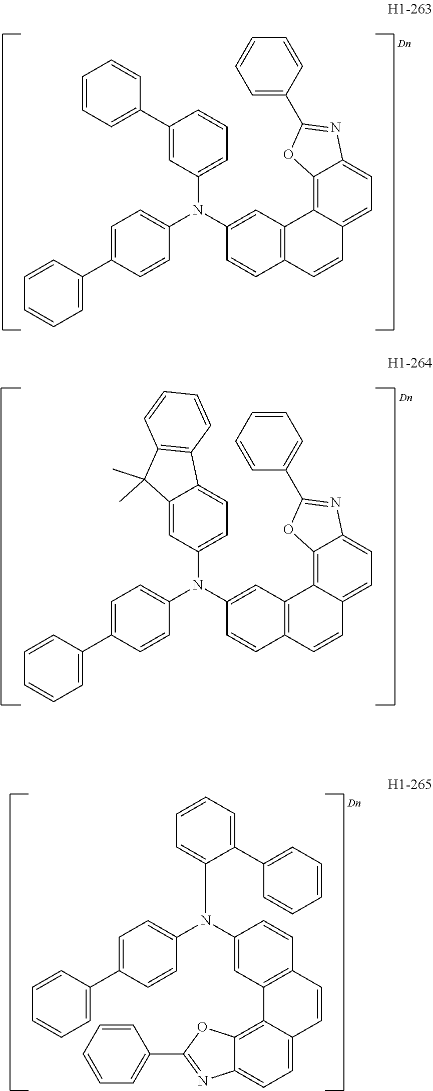 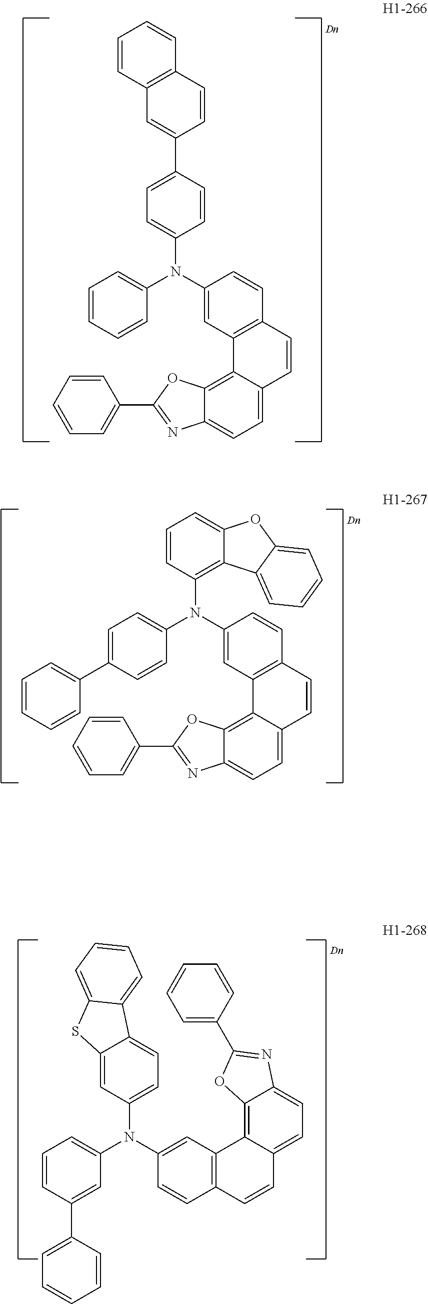 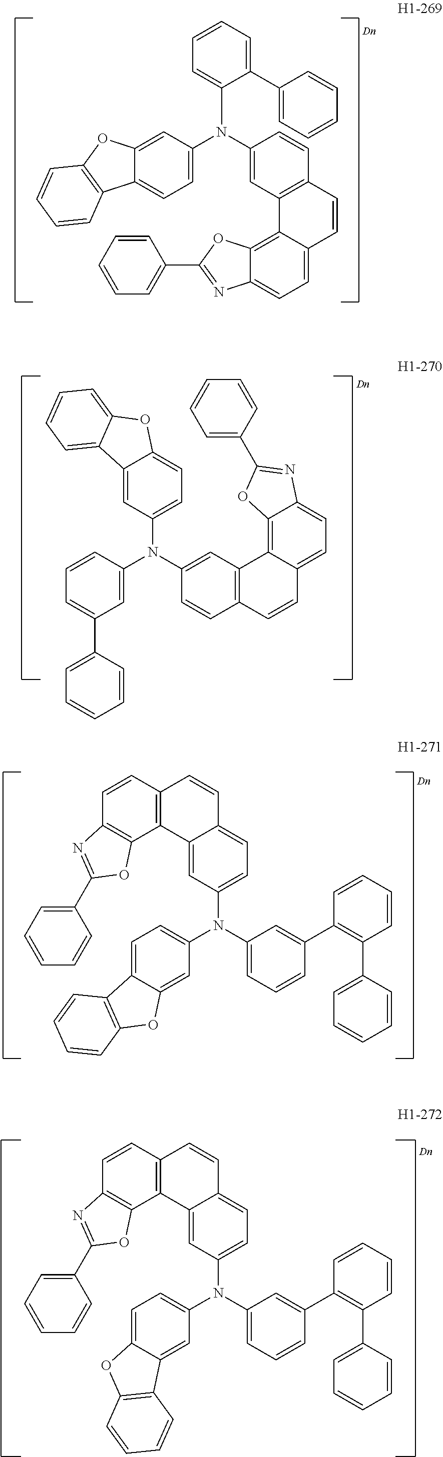 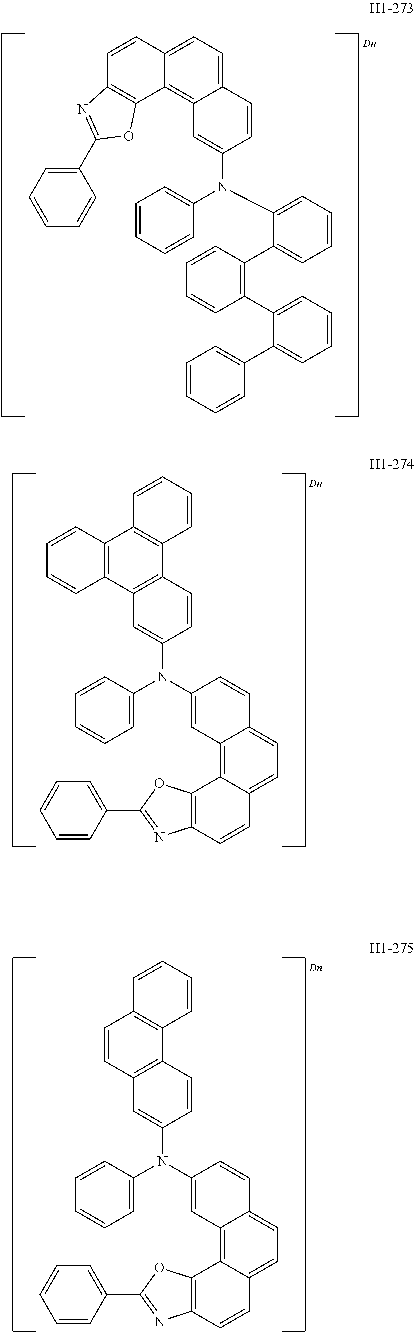 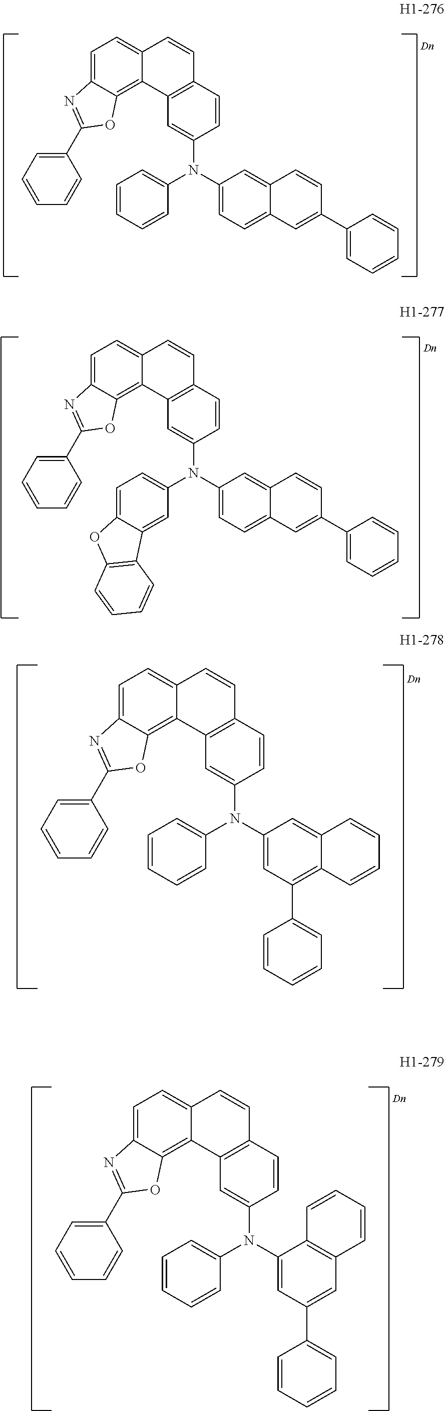 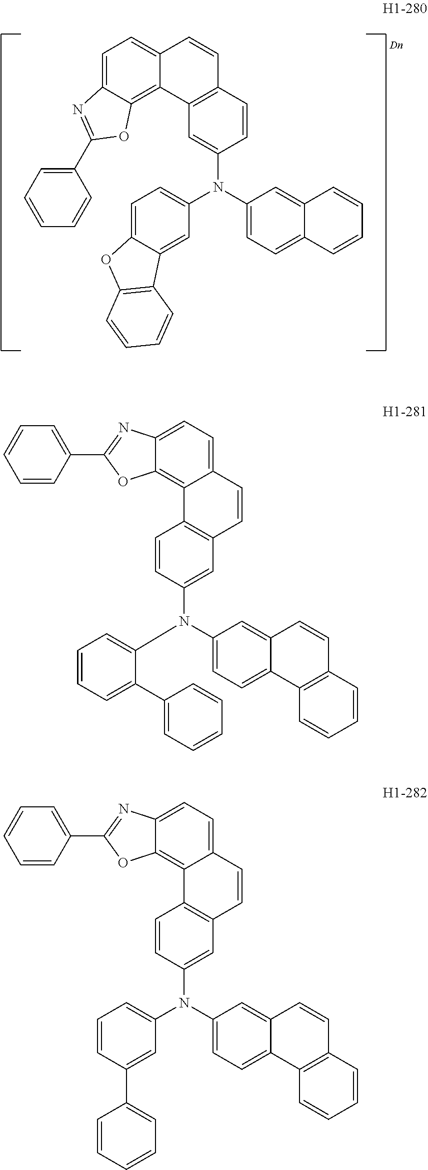 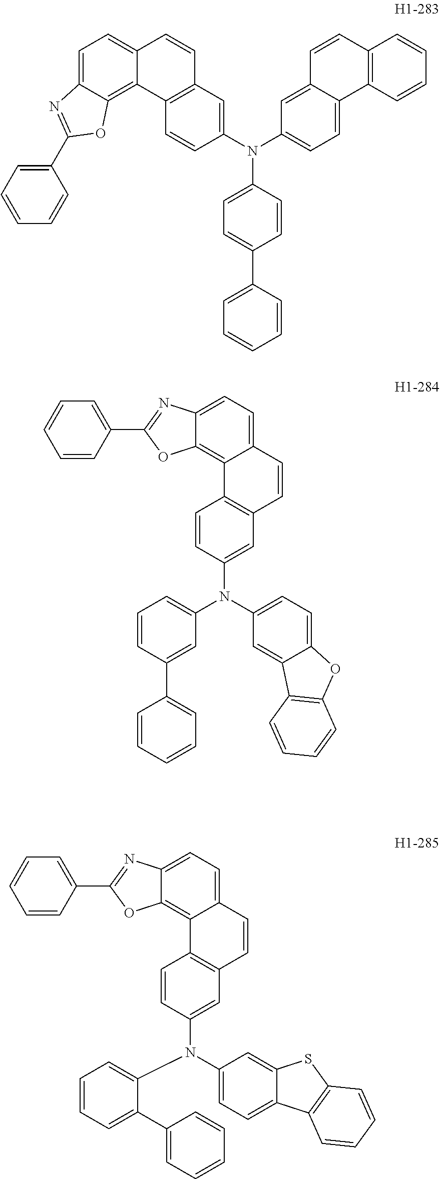 [SMILES: c1ccc(-c2ccc(N(c3ccc4c(ccc5ccccc54)c3)c3ccc4c(ccc5ccc6nc(-c7ccccc7)oc6c54)c3)cc2)cc1 ; c1ccc(-c2cccc(N(c3ccc4c(ccc5ccc6nc(-c7ccccc7)oc6c54)c3)c3ccc4oc5ccccc5c4c3)c2)cc1 ; c1ccc(-c2nc3ccc4ccc5cc(N(c6ccc7c(c6)sc6ccccc67)c6ccccc6-c6ccccc6)ccc5c4c3o2)cc1] 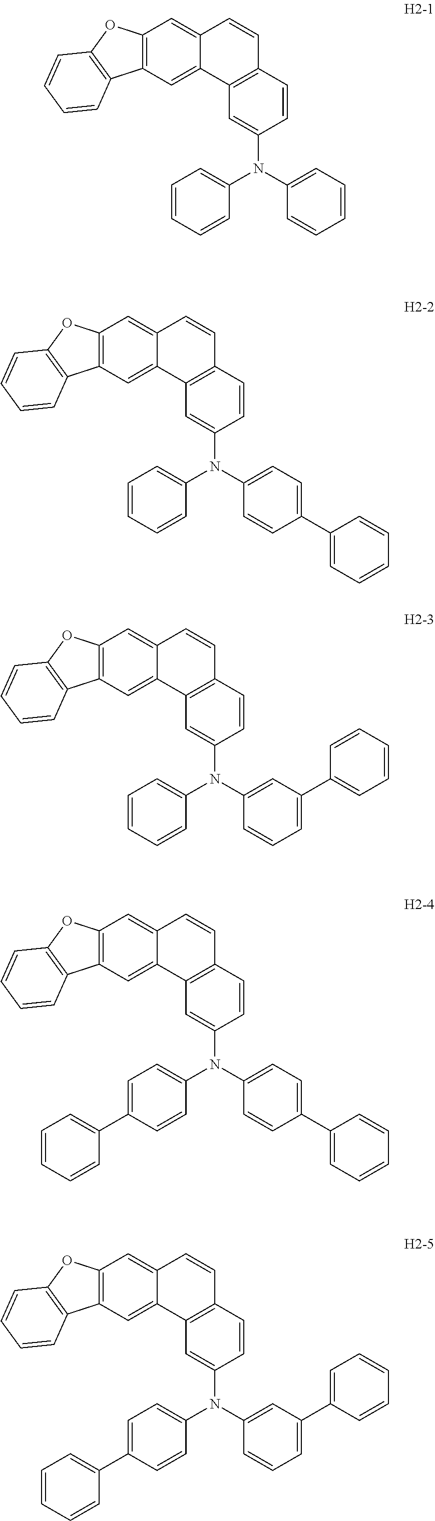 [SMILES: c1ccc(N(c2ccccc2)c2ccc3ccc4cc5oc6ccccc6c5cc4c3c2)cc1 ; c1ccc(-c2ccc(N(c3ccccc3)c3ccc4ccc5cc6oc7ccccc7c6cc5c4c3)cc2)cc1 ; c1ccc(-c2cccc(N(c3ccccc3)c3ccc4ccc5cc6oc7ccccc7c6cc5c4c3)c2)cc1 ; c1ccc(-c2ccc(N(c3ccc(-c4ccccc4)cc3)c3ccc4ccc5cc6oc7ccccc7c6cc5c4c3)cc2)cc1 ; c1ccc(-c2ccc(N(c3cccc(-c4ccccc4)c3)c3ccc4ccc5cc6oc7ccccc7c6cc5c4c3)cc2)cc1] 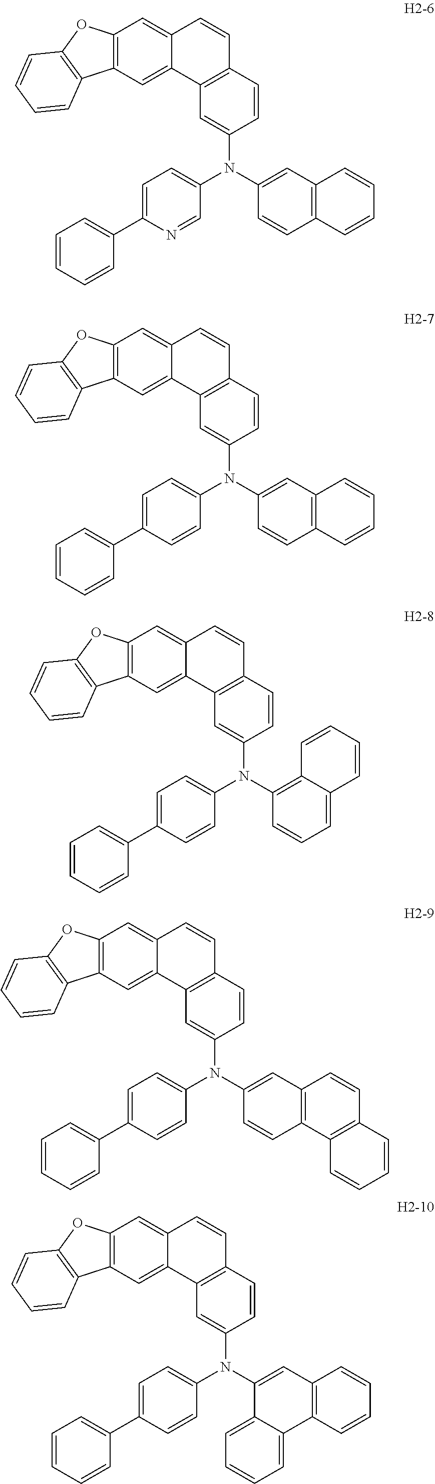 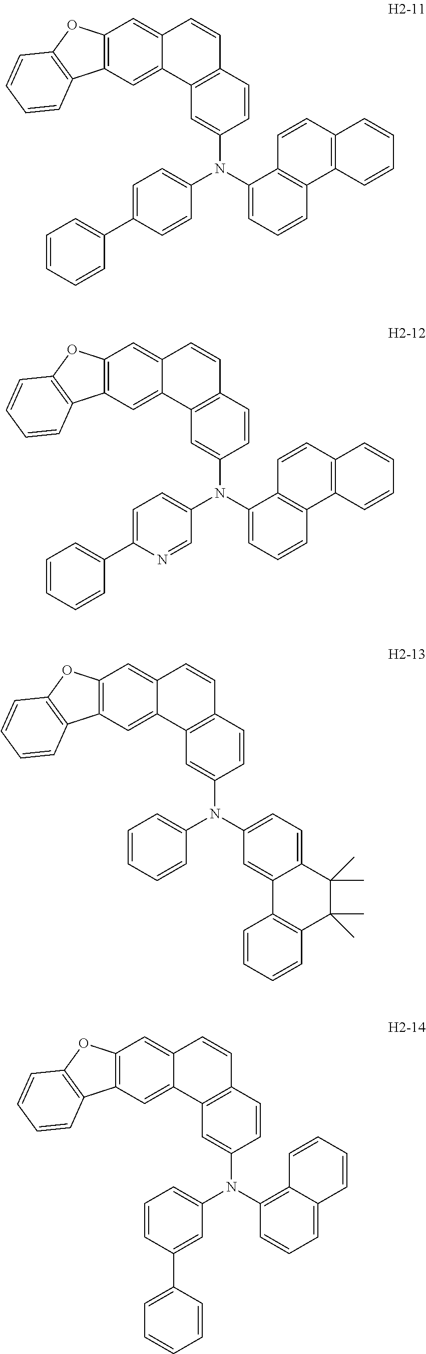 [SMILES: c1ccc(-c2ccc(N(c3ccc4ccc5cc6oc7ccccc7c6cc5c4c3)c3cccc4c3ccc3ccccc34)cc2)cc1 ; c1ccc(-c2ccc(N(c3ccc4ccc5cc6oc7ccccc7c6cc5c4c3)c3cccc4c3ccc3ccccc34)cn2)cc1 ; CC1(C)c2ccccc2-c2cc(N(c3ccccc3)c3ccc4ccc5cc6oc7ccccc7c6cc5c4c3)ccc2C1(C)C ; c1ccc(-c2cccc(N(c3ccc4ccc5cc6oc7ccccc7c6cc5c4c3)c3cccc4ccccc34)c2)cc1] 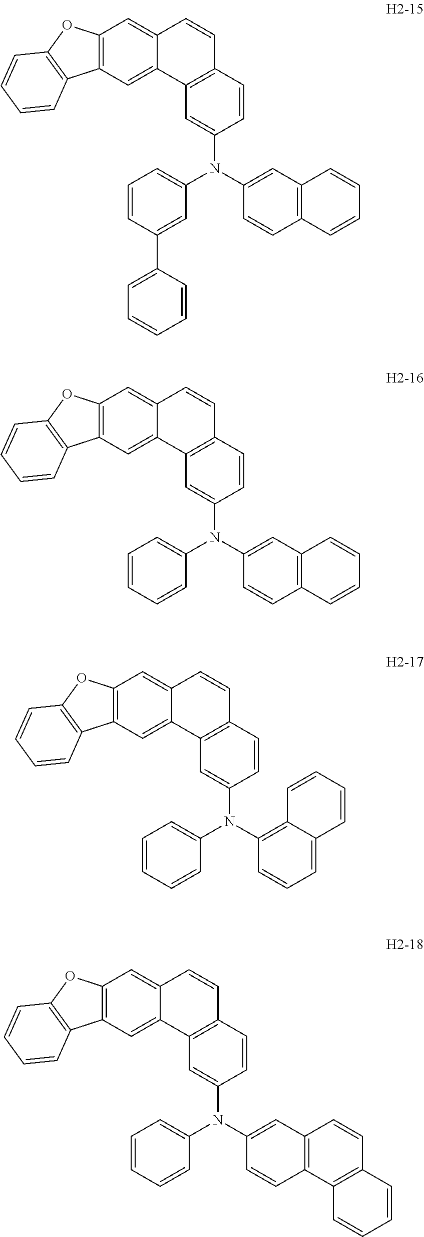 [SMILES: c1ccc(-c2cccc(N(c3ccc4ccccc4c3)c3ccc4ccc5cc6oc7ccccc7c6cc5c4c3)c2)cc1 ; c1ccc(N(c2ccc3ccccc3c2)c2ccc3ccc4cc5oc6ccccc6c5cc4c3c2)cc1 ; c1ccc(N(c2ccc3ccc4cc5oc6ccccc6c5cc4c3c2)c2cccc3ccccc23)cc1 ; c1ccc(N(c2ccc3c(ccc4ccccc43)c2)c2ccc3ccc4cc5oc6ccccc6c5cc4c3c2)cc1] 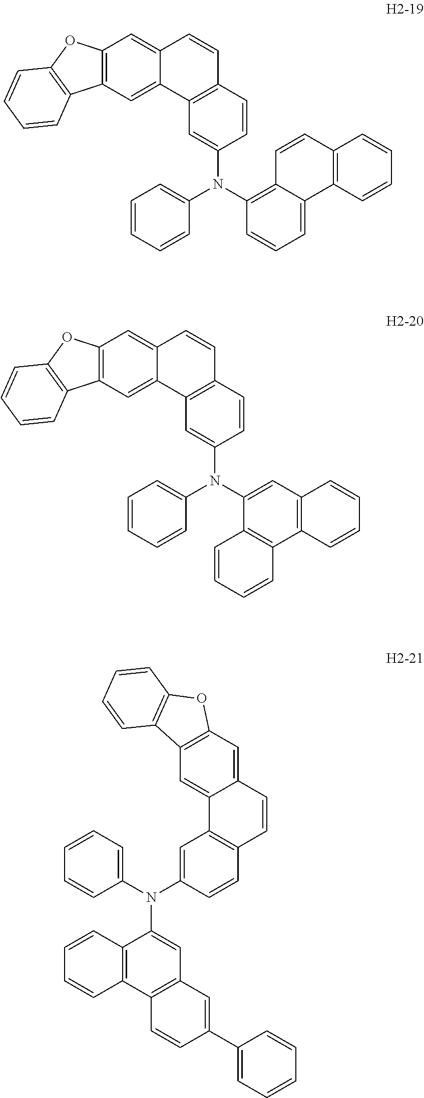 [SMILES: c1ccc(N(c2ccc3ccc4cc5oc6ccccc6c5cc4c3c2)c2cccc3c2ccc2ccccc23)cc1 ; c1ccc(N(c2ccc3ccc4cc5oc6ccccc6c5cc4c3c2)c2cc3ccccc3c3ccccc23)cc1 ; c1ccc(-c2ccc3c(c2)cc(N(c2ccccc2)c2ccc4ccc5cc6oc7ccccc7c6cc5c4c2)c2ccccc23)cc1] 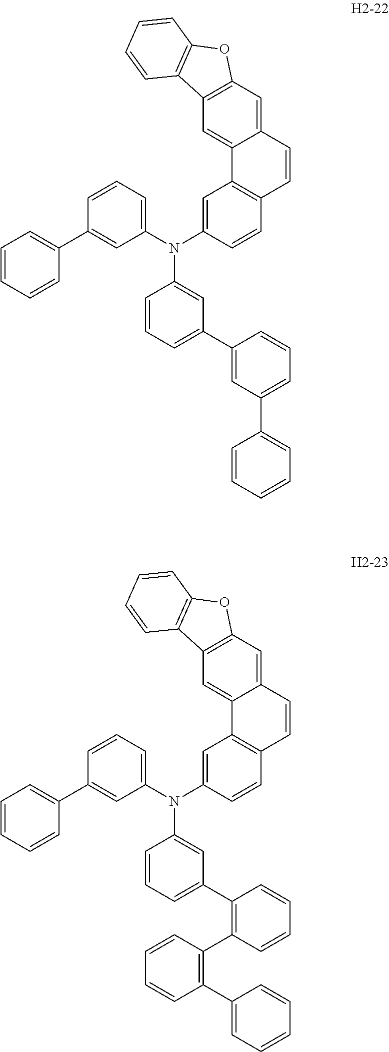 [SMILES: c1ccc(-c2cccc(-c3cccc(N(c4cccc(-c5ccccc5)c4)c4ccc5ccc6cc7oc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6oc7ccccc7c6cc5c4c3)c2)cc1] 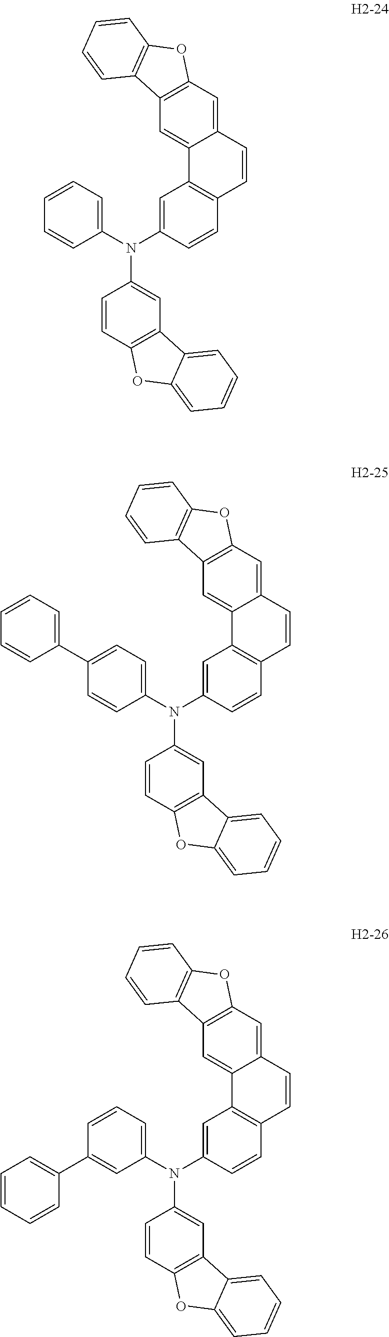 [SMILES: c1ccc(N(c2ccc3ccc4cc5oc6ccccc6c5cc4c3c2)c2ccc3oc4ccccc4c3c2)cc1 ; c1ccc(-c2ccc(N(c3ccc4ccc5cc6oc7ccccc7c6cc5c4c3)c3ccc4oc5ccccc5c4c3)cc2)cc1 ; c1ccc(-c2cccc(N(c3ccc4ccc5cc6oc7ccccc7c6cc5c4c3)c3ccc4oc5ccccc5c4c3)c2)cc1] 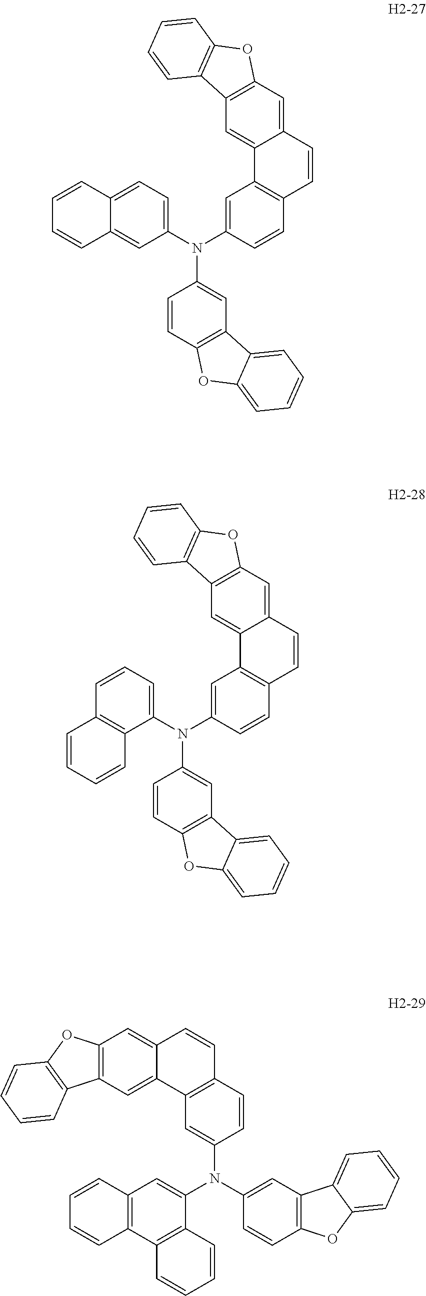 [SMILES: c1ccc2cc(N(c3ccc4ccc5cc6oc7ccccc7c6cc5c4c3)c3ccc4oc5ccccc5c4c3)ccc2c1 ; c1ccc2c(N(c3ccc4ccc5cc6oc7ccccc7c6cc5c4c3)c3ccc4oc5ccccc5c4c3)cccc2c1 ; c1ccc2c(c1)cc(N(c1ccc3ccc4cc5oc6ccccc6c5cc4c3c1)c1ccc3oc4ccccc4c3c1)c1ccccc12] 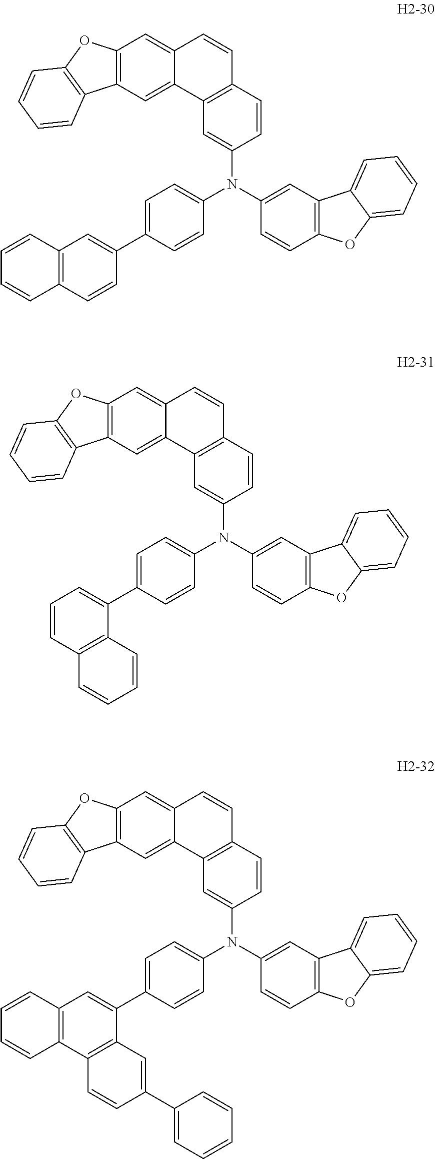 [SMILES: c1ccc2cc(-c3ccc(N(c4ccc5ccc6cc7oc8ccccc8c7cc6c5c4)c4ccc5oc6ccccc6c5c4)cc3)ccc2c1 ; c1ccc2c(-c3ccc(N(c4ccc5ccc6cc7oc8ccccc8c7cc6c5c4)c4ccc5oc6ccccc6c5c4)cc3)cccc2c1 ; c1ccc(-c2ccc3c(c2)c(-c2ccc(N(c4ccc5ccc6cc7oc8ccccc8c7cc6c5c4)c4ccc5oc6ccccc6c5c4)cc2)cc2ccccc23)cc1] 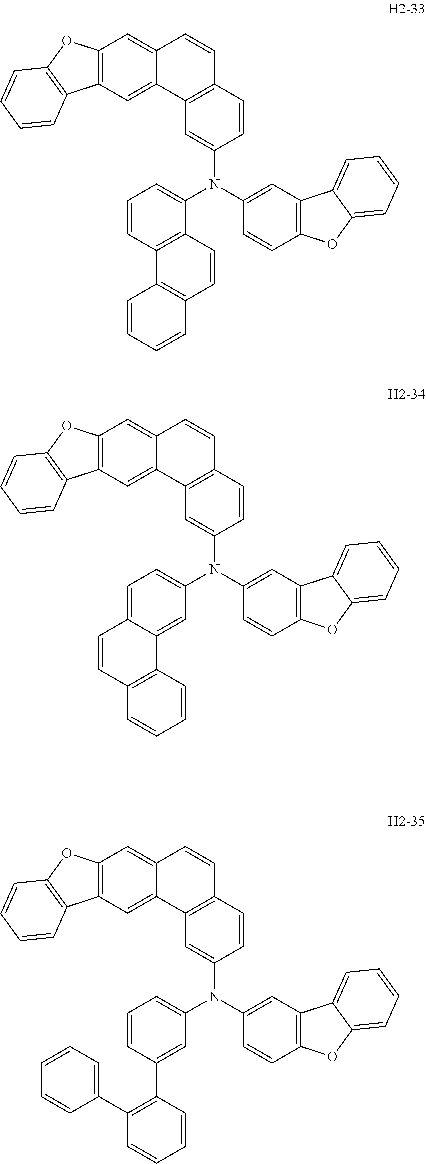 [SMILES: c1ccc2c(c1)ccc1c(N(c3ccc4ccc5cc6oc7ccccc7c6cc5c4c3)c3ccc4oc5ccccc5c4c3)cccc12 ; c1ccc2c(c1)ccc1ccc(N(c3ccc4ccc5cc6oc7ccccc7c6cc5c4c3)c3ccc4oc5ccccc5c4c3)cc12 ; c1ccc(-c2ccccc2-c2cccc(N(c3ccc4ccc5cc6oc7ccccc7c6cc5c4c3)c3ccc4oc5ccccc5c4c3)c2)cc1] 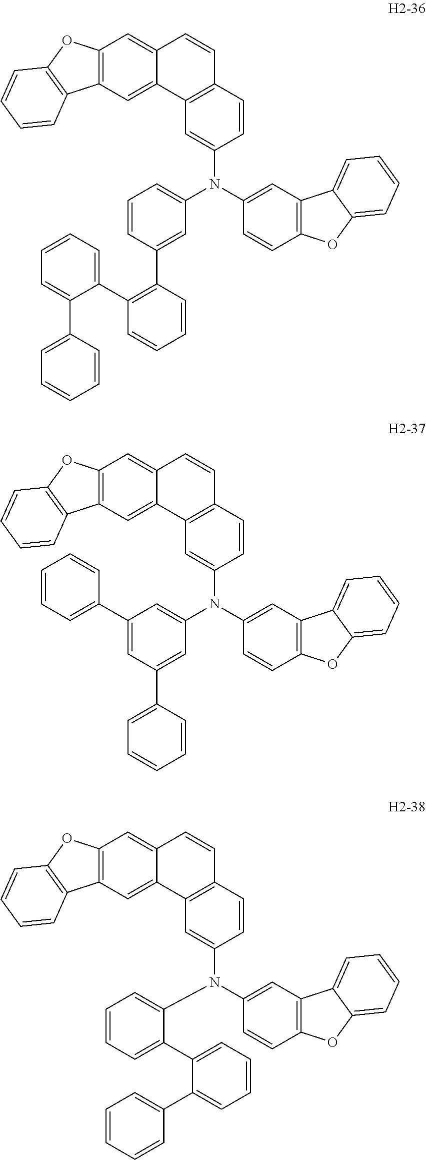 [SMILES: c1ccc(-c2ccccc2-c2ccccc2-c2cccc(N(c3ccc4ccc5cc6oc7ccccc7c6cc5c4c3)c3ccc4oc5ccccc5c4c3)c2)cc1 ; c1ccc(-c2cc(-c3ccccc3)cc(N(c3ccc4ccc5cc6oc7ccccc7c6cc5c4c3)c3ccc4oc5ccccc5c4c3)c2)cc1 ; c1ccc(-c2ccccc2-c2ccccc2N(c2ccc3ccc4cc5oc6ccccc6c5cc4c3c2)c2ccc3oc4ccccc4c3c2)cc1] 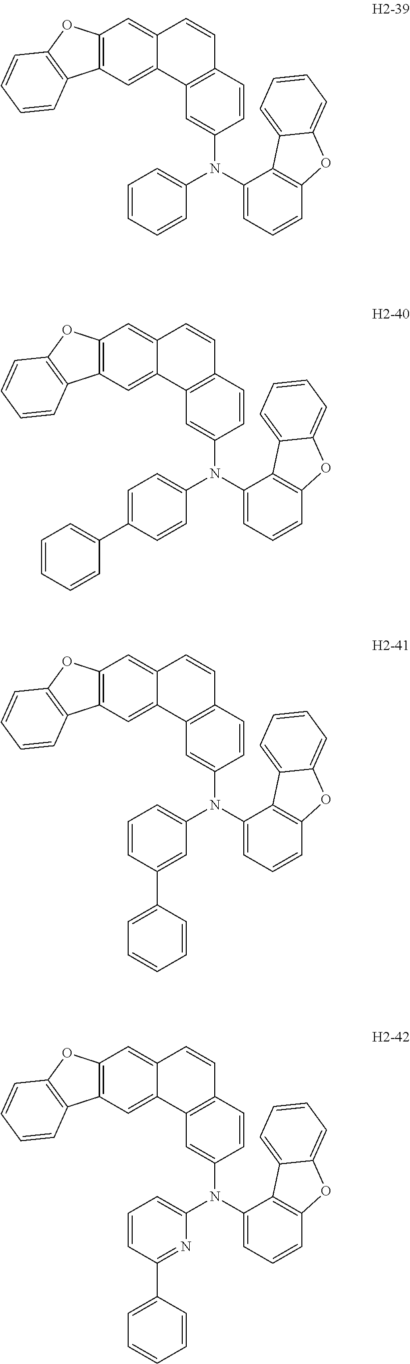 [SMILES: c1ccc(N(c2ccc3ccc4cc5oc6ccccc6c5cc4c3c2)c2cccc3oc4ccccc4c23)cc1 ; c1ccc(-c2ccc(N(c3ccc4ccc5cc6oc7ccccc7c6cc5c4c3)c3cccc4oc5ccccc5c34)cc2)cc1 ; c1ccc(-c2cccc(N(c3ccc4ccc5cc6oc7ccccc7c6cc5c4c3)c3cccc4oc5ccccc5c34)c2)cc1 ; c1ccc(-c2cccc(N(c3ccc4ccc5cc6oc7ccccc7c6cc5c4c3)c3cccc4oc5ccccc5c34)n2)cc1] 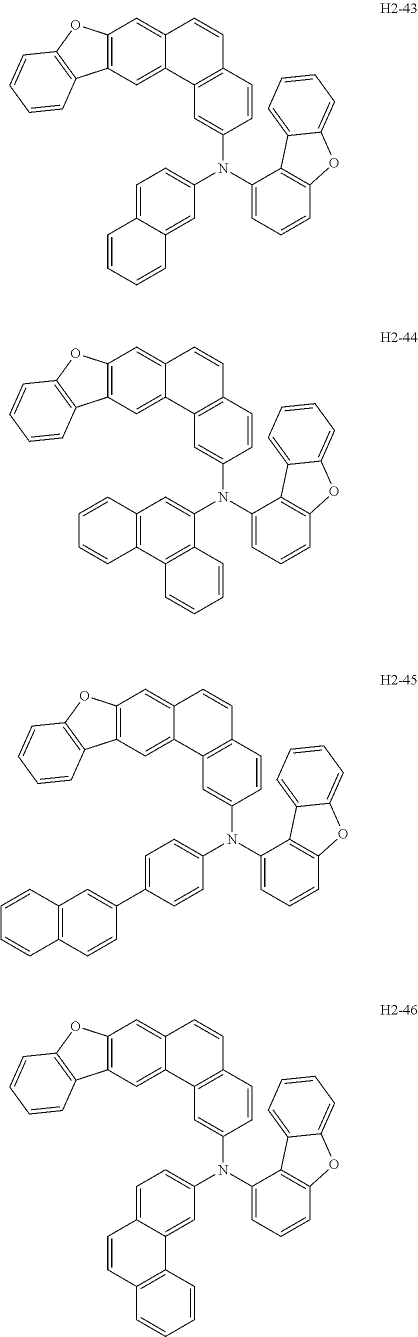 [SMILES: c1ccc2cc(N(c3ccc4ccc5cc6oc7ccccc7c6cc5c4c3)c3cccc4oc5ccccc5c34)ccc2c1 ; c1ccc2c(c1)cc(N(c1ccc3ccc4cc5oc6ccccc6c5cc4c3c1)c1cccc3oc4ccccc4c13)c1ccccc12 ; c1ccc2cc(-c3ccc(N(c4ccc5ccc6cc7oc8ccccc8c7cc6c5c4)c4cccc5oc6ccccc6c45)cc3)ccc2c1 ; c1ccc2c(c1)ccc1ccc(N(c3ccc4ccc5cc6oc7ccccc7c6cc5c4c3)c3cccc4oc5ccccc5c34)cc12] 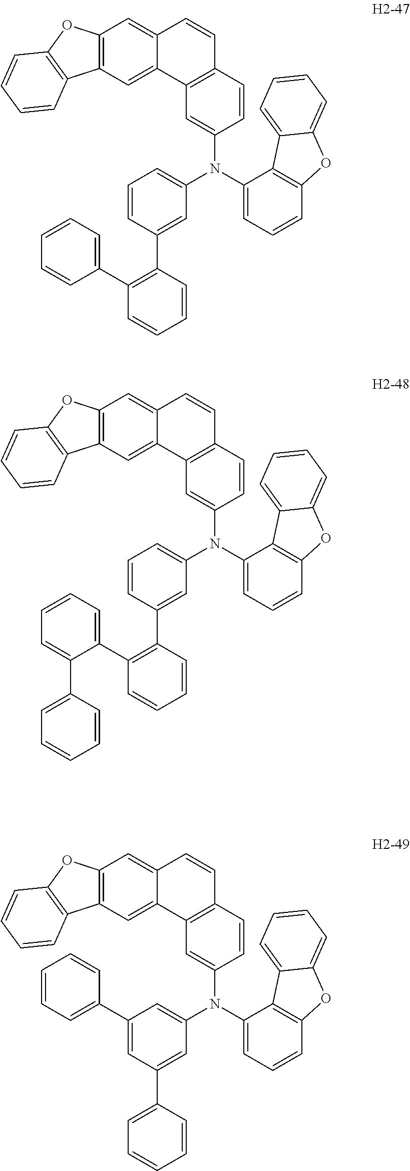 [SMILES: c1ccc(-c2ccccc2-c2cccc(N(c3ccc4ccc5cc6oc7ccccc7c6cc5c4c3)c3cccc4oc5ccccc5c34)c2)cc1 ; c1ccc(-c2ccccc2-c2ccccc2-c2cccc(N(c3ccc4ccc5cc6oc7ccccc7c6cc5c4c3)c3cccc4oc5ccccc5c34)c2)cc1 ; c1ccc(-c2cc(-c3ccccc3)cc(N(c3ccc4ccc5cc6oc7ccccc7c6cc5c4c3)c3cccc4oc5ccccc5c34)c2)cc1] 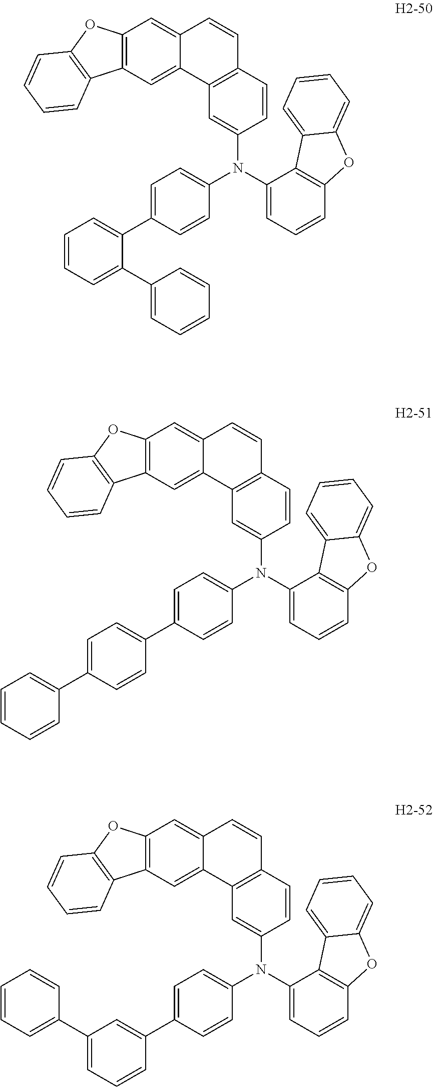 [SMILES: c1ccc(-c2ccccc2-c2ccc(N(c3ccc4ccc5cc6oc7ccccc7c6cc5c4c3)c3cccc4oc5ccccc5c34)cc2)cc1 ; c1ccc(-c2ccc(-c3ccc(N(c4ccc5ccc6cc7oc8ccccc8c7cc6c5c4)c4cccc5oc6ccccc6c45)cc3)cc2)cc1 ; c1ccc(-c2cccc(-c3ccc(N(c4ccc5ccc6cc7oc8ccccc8c7cc6c5c4)c4cccc5oc6ccccc6c45)cc3)c2)cc1] 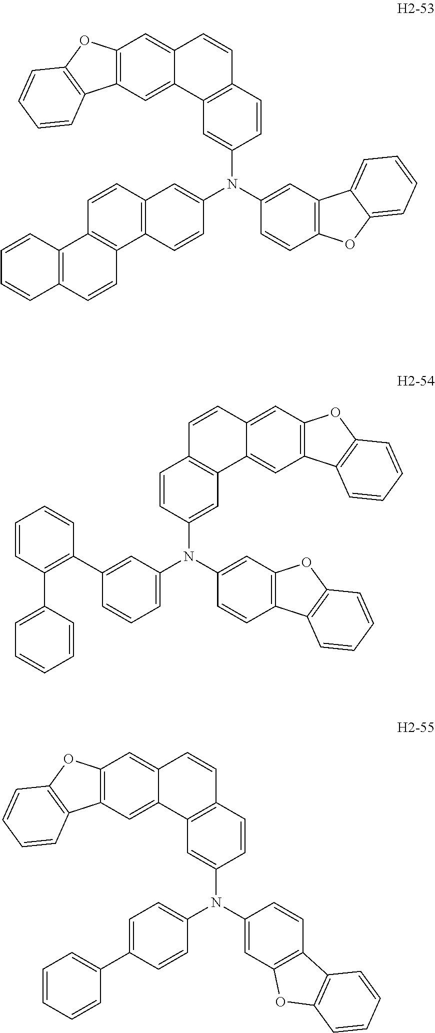 [SMILES: c1ccc2c(c1)ccc1c3ccc(N(c4ccc5ccc6cc7oc8ccccc8c7cc6c5c4)c4ccc5oc6ccccc6c5c4)cc3ccc21 ; c1ccc(-c2ccccc2-c2cccc(N(c3ccc4c(c3)oc3ccccc34)c3ccc4ccc5cc6oc7ccccc7c6cc5c4c3)c2)cc1 ; c1ccc(-c2ccc(N(c3ccc4c(c3)oc3ccccc34)c3ccc4ccc5cc6oc7ccccc7c6cc5c4c3)cc2)cc1] 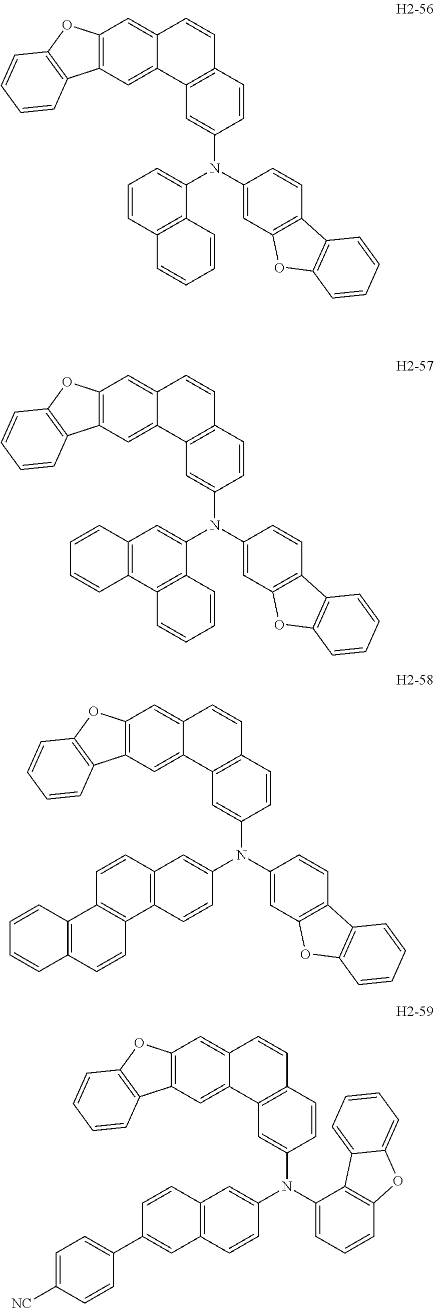 [SMILES: c1ccc2c(N(c3ccc4c(c3)oc3ccccc34)c3ccc4ccc5cc6oc7ccccc7c6cc5c4c3)cccc2c1 ; c1ccc2c(c1)cc(N(c1ccc3c(c1)oc1ccccc13)c1ccc3ccc4cc5oc6ccccc6c5cc4c3c1)c1ccccc12 ; c1ccc2c(c1)ccc1c3ccc(N(c4ccc5c(c4)oc4ccccc45)c4ccc5ccc6cc7oc8ccccc8c7cc6c5c4)cc3ccc21 ; N#Cc1ccc(-c2ccc3cc(N(c4ccc5ccc6cc7oc8ccccc8c7cc6c5c4)c4cccc5oc6ccccc6c45)ccc3c2)cc1] 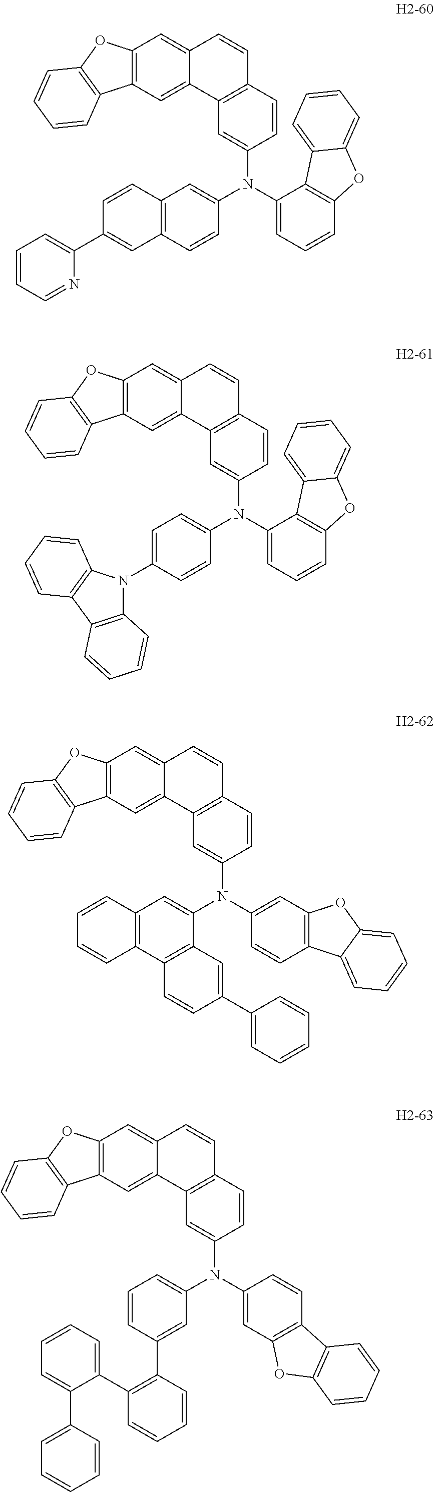 [SMILES: c1ccc(-c2ccc3cc(N(c4ccc5ccc6cc7oc8ccccc8c7cc6c5c4)c4cccc5oc6ccccc6c45)ccc3c2)nc1 ; c1ccc2c(c1)oc1cc3ccc4ccc(N(c5ccc(-n6c7ccccc7c7ccccc76)cc5)c5cccc6oc7ccccc7c56)cc4c3cc12 ; c1ccc(-c2ccc3c(c2)c(N(c2ccc4c(c2)oc2ccccc24)c2ccc4ccc5cc6oc7ccccc7c6cc5c4c2)cc2ccccc23)cc1 ; c1ccc(-c2ccccc2-c2ccccc2-c2cccc(N(c3ccc4c(c3)oc3ccccc34)c3ccc4ccc5cc6oc7ccccc7c6cc5c4c3)c2)cc1] 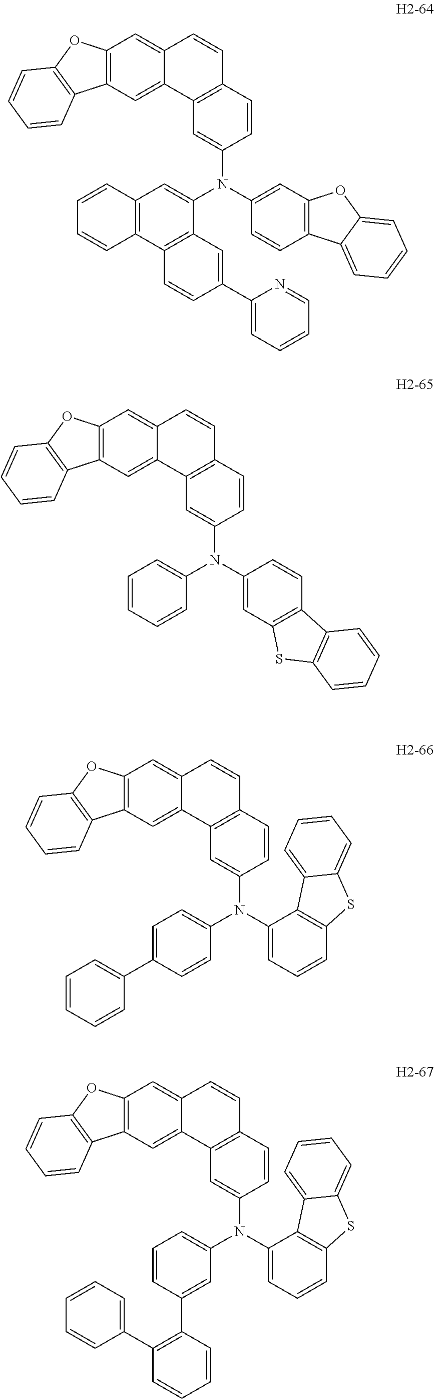 [SMILES: c1ccc(-c2ccc3c(c2)c(N(c2ccc4c(c2)oc2ccccc24)c2ccc4ccc5cc6oc7ccccc7c6cc5c4c2)cc2ccccc23)nc1 ; c1ccc(N(c2ccc3c(c2)sc2ccccc23)c2ccc3ccc4cc5oc6ccccc6c5cc4c3c2)cc1 ; c1ccc(-c2ccc(N(c3ccc4ccc5cc6oc7ccccc7c6cc5c4c3)c3cccc4sc5ccccc5c34)cc2)cc1 ; c1ccc(-c2ccccc2-c2cccc(N(c3ccc4ccc5cc6oc7ccccc7c6cc5c4c3)c3cccc4sc5ccccc5c34)c2)cc1] 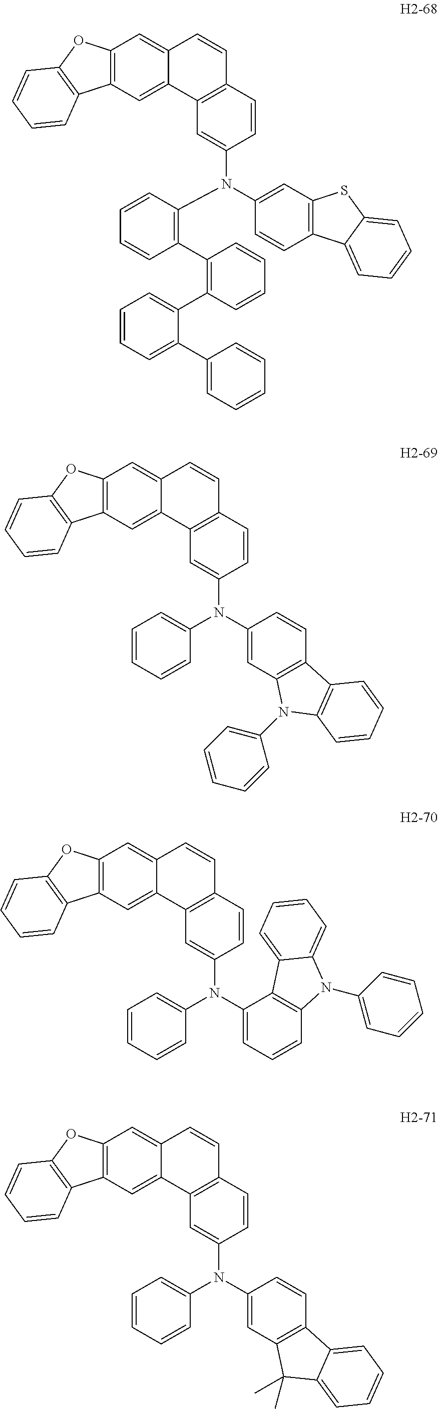 [SMILES: c1ccc(-c2ccccc2-c2ccccc2-c2ccccc2N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2ccc3c4ccccc4n(-c4ccccc4)c3c2)cc1 ; c1ccc(N(c2ccc3ccc4cc5oc6ccccc6c5cc4c3c2)c2cccc3c2c2ccccc2n3-c2ccccc2)cc1 ; CC1(C)c2ccccc2-c2ccc(N(c3ccccc3)c3ccc4ccc5cc6oc7ccccc7c6cc5c4c3)cc21] 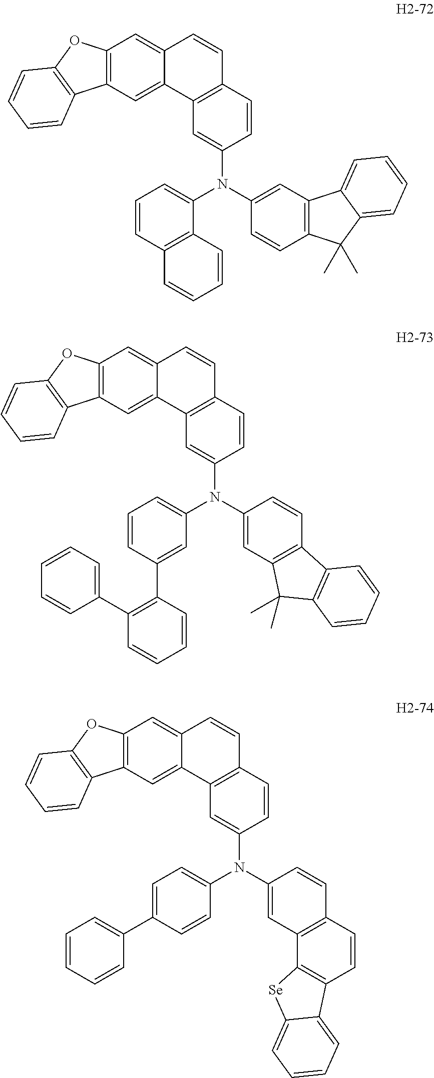 [SMILES: CC1(C)c2ccccc2-c2cc(N(c3ccc4ccc5cc6oc7ccccc7c6cc5c4c3)c3cccc4ccccc34)ccc21 ; CC1(C)c2ccccc2-c2ccc(N(c3cccc(-c4ccccc4-c4ccccc4)c3)c3ccc4ccc5cc6oc7ccccc7c6cc5c4c3)cc21 ; c1ccc(-c2ccc(N(c3ccc4ccc5cc6oc7ccccc7c6cc5c4c3)c3ccc4ccc5c6ccccc6[se]c5c4c3)cc2)cc1] 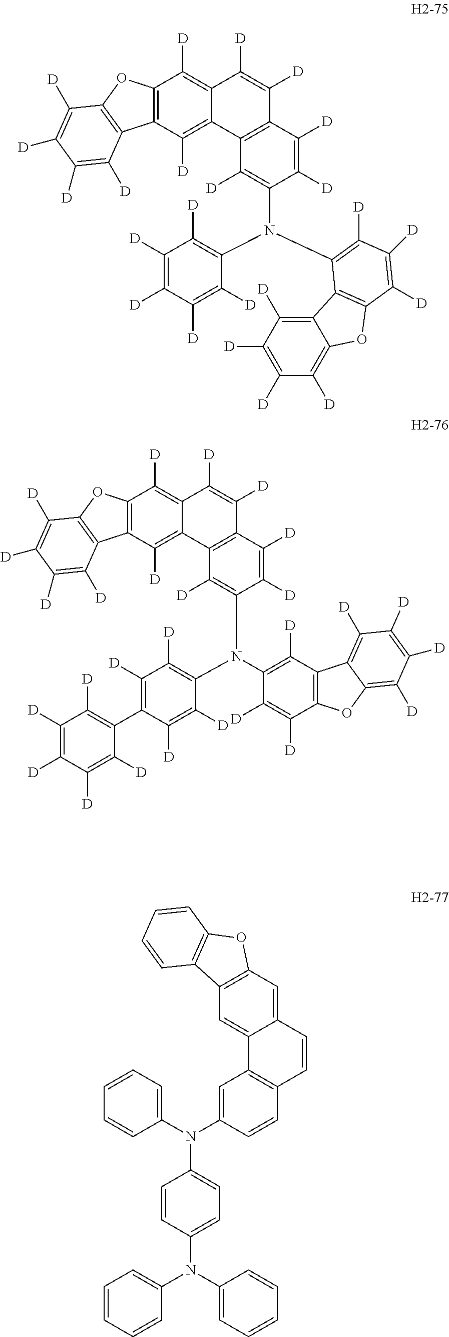 [SMILES: [2H]c1c([2H])c([2H])c(N(c2c([2H])c([2H])c3c([2H])c([2H])c4c([2H])c5oc6c([2H])c([2H])c([2H])c([2H])c6c5c([2H])c4c3c2[2H])c2c([2H])c([2H])c([2H])c3oc4c([2H])c([2H])c([2H])c([2H])c4c23)c([2H])c1[2H] ; [2H]c1c([2H])c([2H])c(-c2c([2H])c([2H])c(N(c3c([2H])c([2H])c4oc5c([2H])c([2H])c([2H])c([2H])c5c4c3[2H])c3c([2H])c([2H])c4c([2H])c([2H])c5c([2H])c6oc7c([2H])c([2H])c([2H])c([2H])c7c6c([2H])c5c4c3[2H])c([2H])c2[2H])c([2H])c1[2H] ; c1ccc(N(c2ccccc2)c2ccc(N(c3ccccc3)c3ccc4ccc5cc6oc7ccccc7c6cc5c4c3)cc2)cc1] 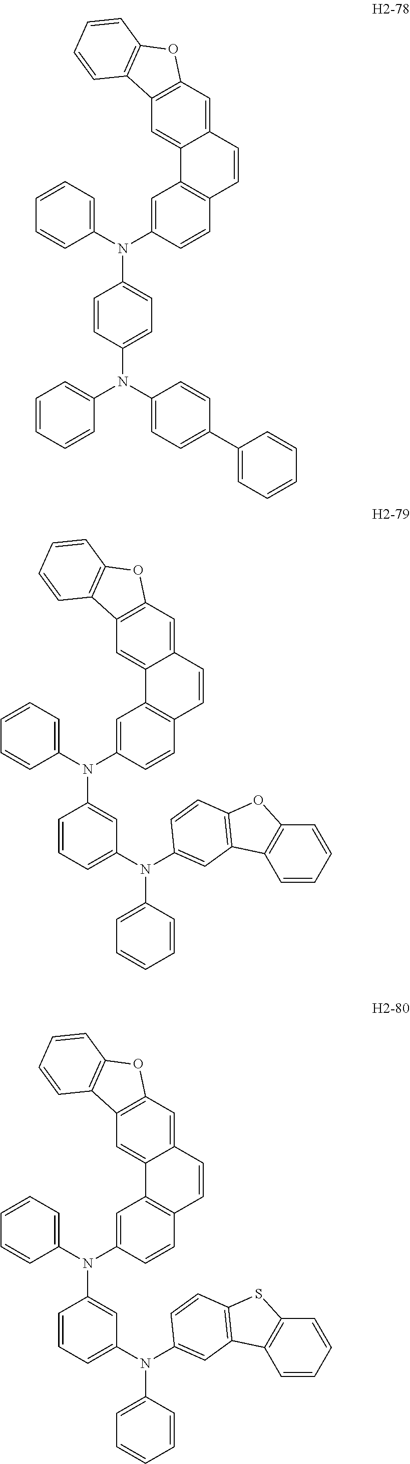 [SMILES: c1ccc(-c2ccc(N(c3ccccc3)c3ccc(N(c4ccccc4)c4ccc5ccc6cc7oc8ccccc8c7cc6c5c4)cc3)cc2)cc1 ; c1ccc(N(c2cccc(N(c3ccccc3)c3ccc4oc5ccccc5c4c3)c2)c2ccc3ccc4cc5oc6ccccc6c5cc4c3c2)cc1 ; c1ccc(N(c2cccc(N(c3ccccc3)c3ccc4sc5ccccc5c4c3)c2)c2ccc3ccc4cc5oc6ccccc6c5cc4c3c2)cc1] 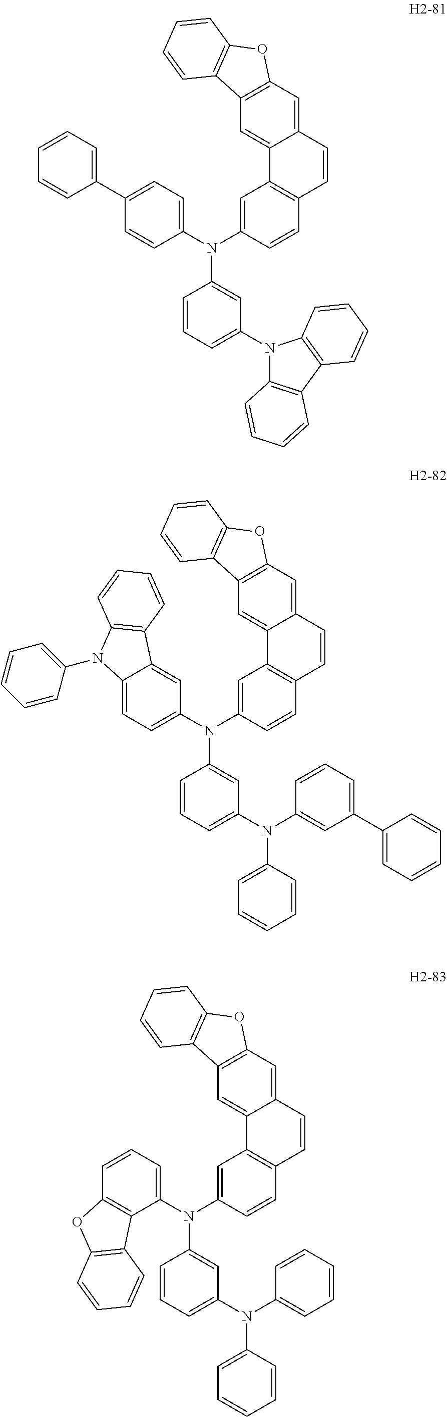 [SMILES: c1ccc(-c2ccc(N(c3cccc(-n4c5ccccc5c5ccccc54)c3)c3ccc4ccc5cc6oc7ccccc7c6cc5c4c3)cc2)cc1 ; c1ccc(-c2cccc(N(c3ccccc3)c3cccc(N(c4ccc5ccc6cc7oc8ccccc8c7cc6c5c4)c4ccc5c(c4)c4ccccc4n5-c4ccccc4)c3)c2)cc1 ; c1ccc(N(c2ccccc2)c2cccc(N(c3ccc4ccc5cc6oc7ccccc7c6cc5c4c3)c3cccc4oc5ccccc5c34)c2)cc1] 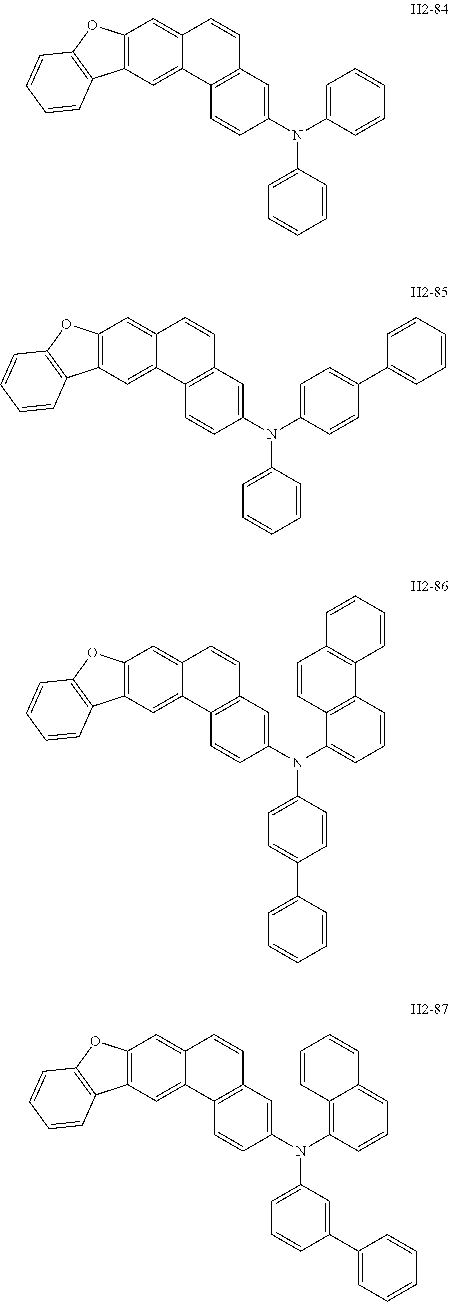 [SMILES: c1ccc(N(c2ccccc2)c2ccc3c(ccc4cc5oc6ccccc6c5cc43)c2)cc1 ; c1ccc(-c2ccc(N(c3ccccc3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cccc4c3ccc3ccccc34)cc2)cc1 ; c1ccc(-c2cccc(N(c3ccc4c(ccc5cc6oc7ccccc7c6cc54)c3)c3cccc4ccccc34)c2)cc1] 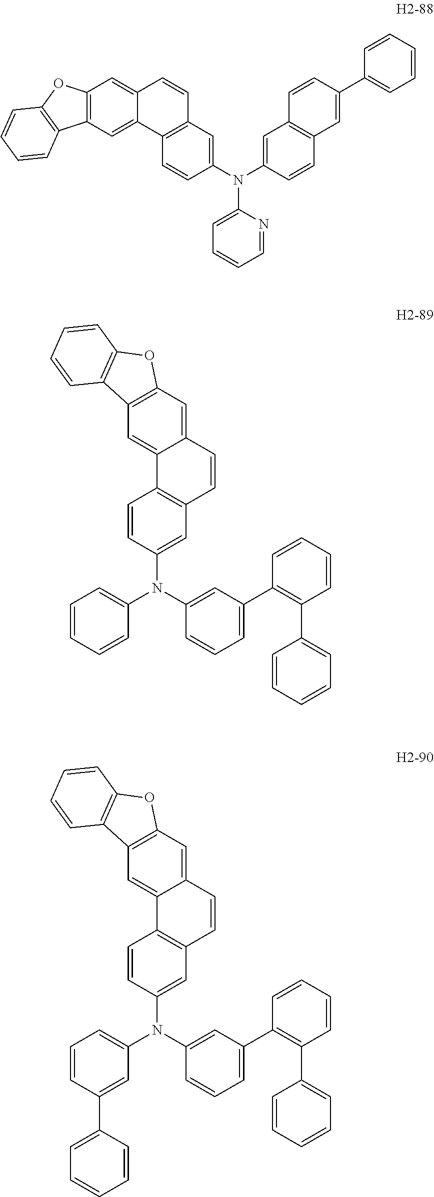 [SMILES: c1ccc(-c2ccc3cc(N(c4ccc5c(ccc6cc7oc8ccccc8c7cc65)c4)c4ccccn4)ccc3c2)cc1 ; c1ccc(-c2ccccc2-c2cccc(N(c3ccccc3)c3ccc4c(ccc5cc6oc7ccccc7c6cc54)c3)c2)cc1 ; c1ccc(-c2cccc(N(c3cccc(-c4ccccc4-c4ccccc4)c3)c3ccc4c(ccc5cc6oc7ccccc7c6cc54)c3)c2)cc1] 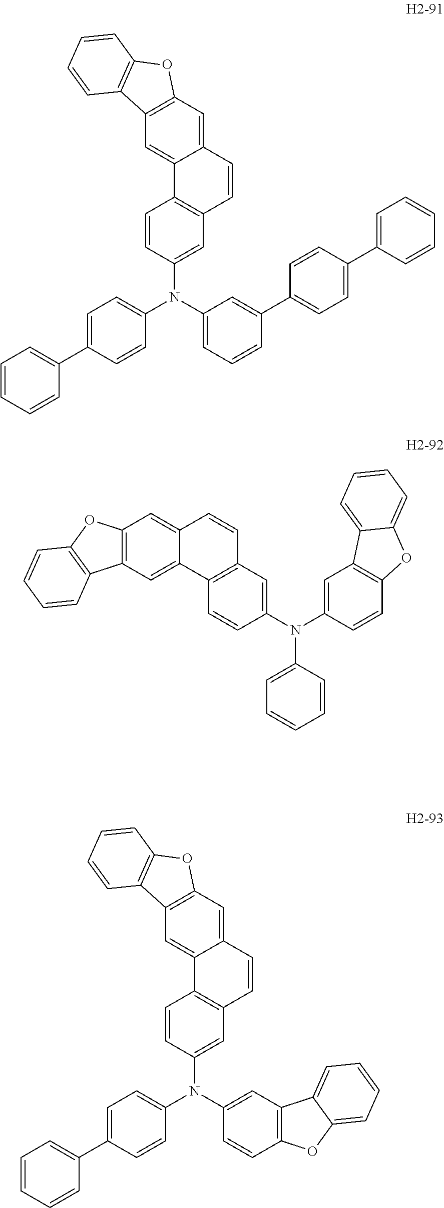 [SMILES: c1ccc(-c2ccc(-c3cccc(N(c4ccc(-c5ccccc5)cc4)c4ccc5c(ccc6cc7oc8ccccc8c7cc65)c4)c3)cc2)cc1 ; c1ccc(N(c2ccc3c(ccc4cc5oc6ccccc6c5cc43)c2)c2ccc3oc4ccccc4c3c2)cc1 ; c1ccc(-c2ccc(N(c3ccc4c(ccc5cc6oc7ccccc7c6cc54)c3)c3ccc4oc5ccccc5c4c3)cc2)cc1] 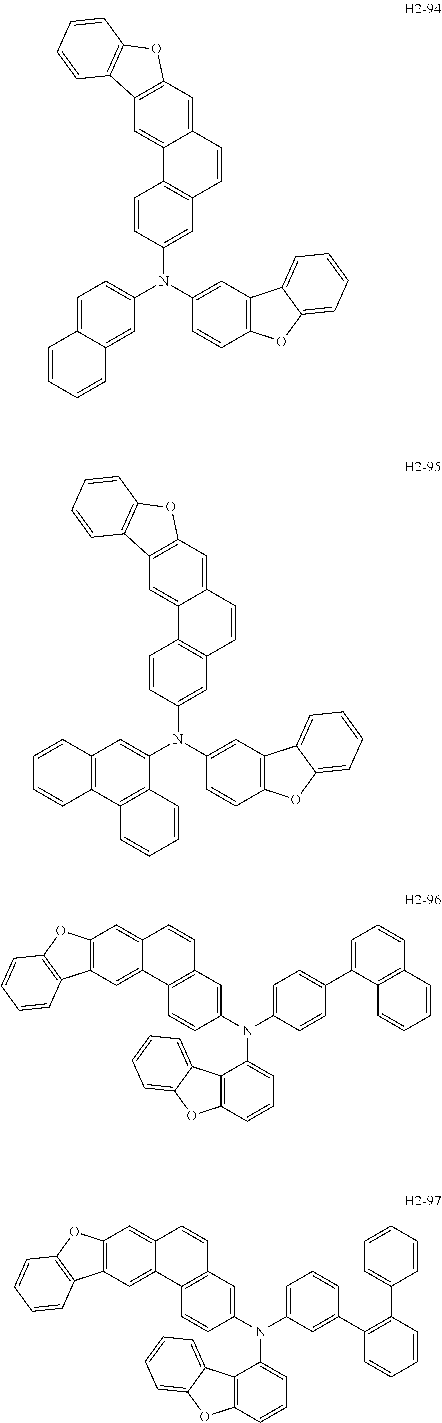 [SMILES: c1ccc2cc(N(c3ccc4c(ccc5cc6oc7ccccc7c6cc54)c3)c3ccc4oc5ccccc5c4c3)ccc2c1 ; c1ccc2c(c1)cc(N(c1ccc3c(ccc4cc5oc6ccccc6c5cc43)c1)c1ccc3oc4ccccc4c3c1)c1ccccc12 ; c1ccc2c(-c3ccc(N(c4ccc5c(ccc6cc7oc8ccccc8c7cc65)c4)c4cccc5oc6ccccc6c45)cc3)cccc2c1 ; c1ccc(-c2ccccc2-c2cccc(N(c3ccc4c(ccc5cc6oc7ccccc7c6cc54)c3)c3cccc4oc5ccccc5c34)c2)cc1] 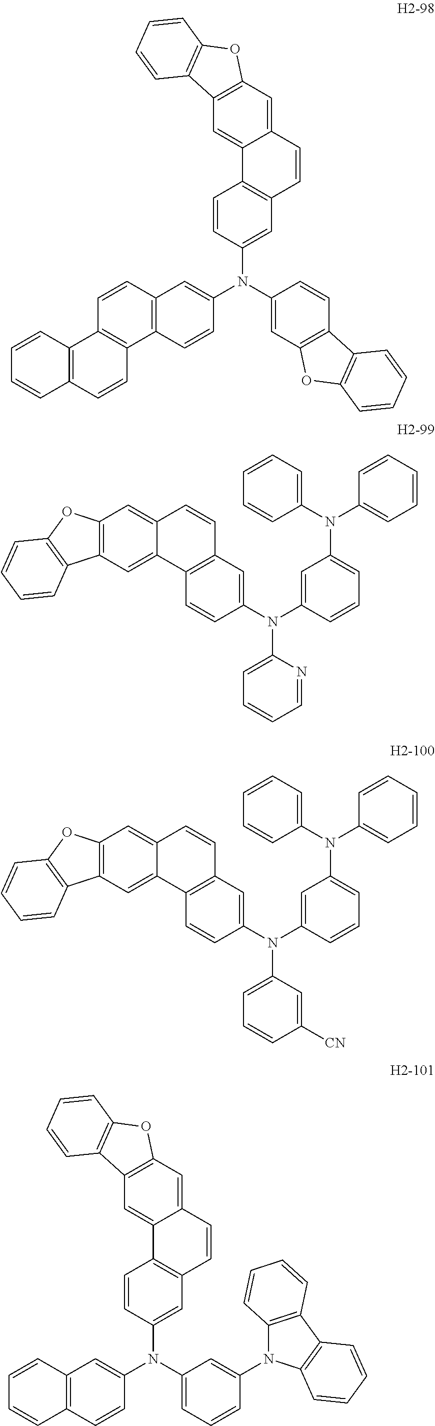 [SMILES: c1ccc2c(c1)ccc1c3ccc(N(c4ccc5c(ccc6cc7oc8ccccc8c7cc65)c4)c4ccc5c(c4)oc4ccccc45)cc3ccc21 ; c1ccc(N(c2ccccc2)c2cccc(N(c3ccc4c(ccc5cc6oc7ccccc7c6cc54)c3)c3ccccn3)c2)cc1 ; N#Cc1cccc(N(c2cccc(N(c3ccccc3)c3ccccc3)c2)c2ccc3c(ccc4cc5oc6ccccc6c5cc43)c2)c1 ; c1cc(N(c2ccc3ccccc3c2)c2ccc3c(ccc4cc5oc6ccccc6c5cc43)c2)cc(-n2c3ccccc3c3ccccc32)c1] 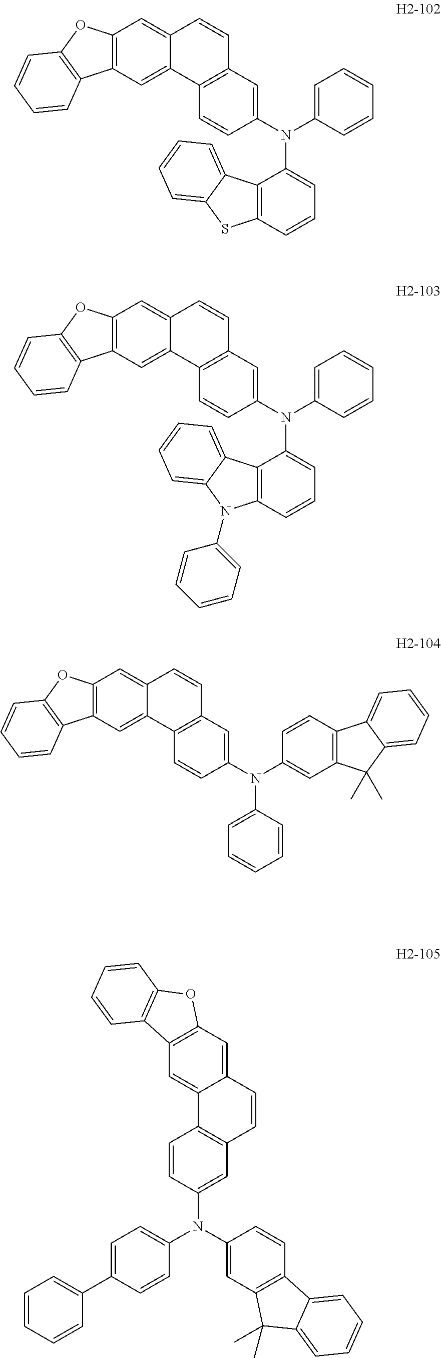 [SMILES: c1ccc(N(c2ccc3c(ccc4cc5oc6ccccc6c5cc43)c2)c2cccc3sc4ccccc4c23)cc1 ; c1ccc(N(c2ccc3c(ccc4cc5oc6ccccc6c5cc43)c2)c2cccc3c2c2ccccc2n3-c2ccccc2)cc1 ; CC1(C)c2ccccc2-c2ccc(N(c3ccccc3)c3ccc4c(ccc5cc6oc7ccccc7c6cc54)c3)cc21 ; CC1(C)c2ccccc2-c2ccc(N(c3ccc(-c4ccccc4)cc3)c3ccc4c(ccc5cc6oc7ccccc7c6cc54)c3)cc21] 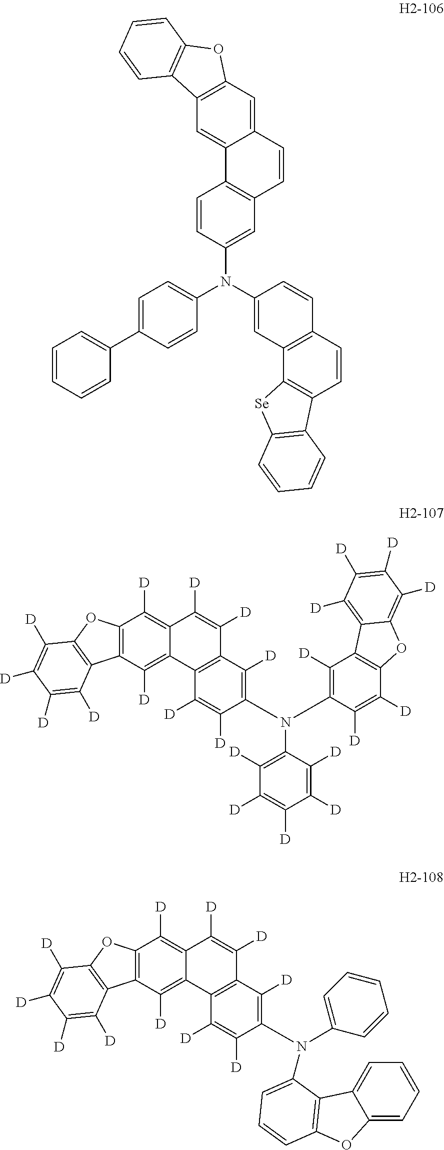 [SMILES: c1ccc(-c2ccc(N(c3ccc4c(ccc5cc6oc7ccccc7c6cc54)c3)c3ccc4ccc5c6ccccc6[se]c5c4c3)cc2)cc1 ; [2H]c1c([2H])c([2H])c(N(c2c([2H])c([2H])c3c(c2[2H])c([2H])c([2H])c2c([2H])c4oc5c([2H])c([2H])c([2H])c([2H])c5c4c([2H])c23)c2c([2H])c([2H])c3oc4c([2H])c([2H])c([2H])c([2H])c4c3c2[2H])c([2H])c1[2H] ; [2H]c1c([2H])c([2H])c2c(oc3c([2H])c4c([2H])c([2H])c5c([2H])c(N(c6ccccc6)c6cccc7oc8ccccc8c67)c([2H])c([2H])c5c4c([2H])c32)c1[2H]] 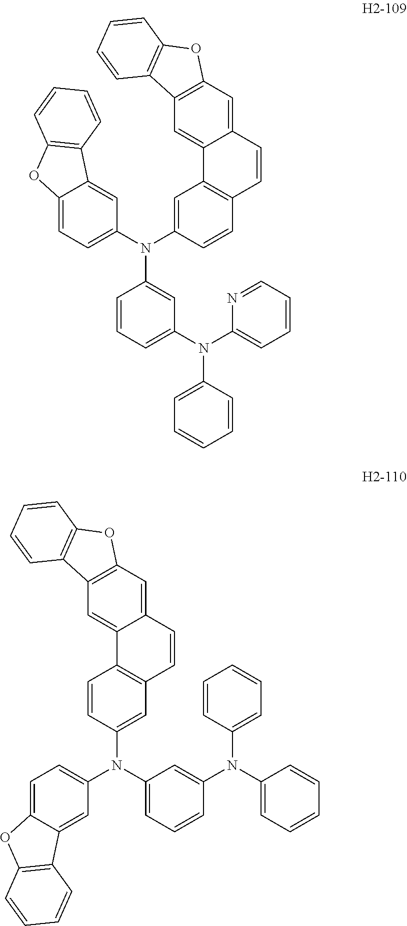 [SMILES: c1ccc(N(c2cccc(N(c3ccc4ccc5cc6oc7ccccc7c6cc5c4c3)c3ccc4oc5ccccc5c4c3)c2)c2ccccn2)cc1 ; c1ccc(N(c2ccccc2)c2cccc(N(c3ccc4c(ccc5cc6oc7ccccc7c6cc54)c3)c3ccc4oc5ccccc5c4c3)c2)cc1] 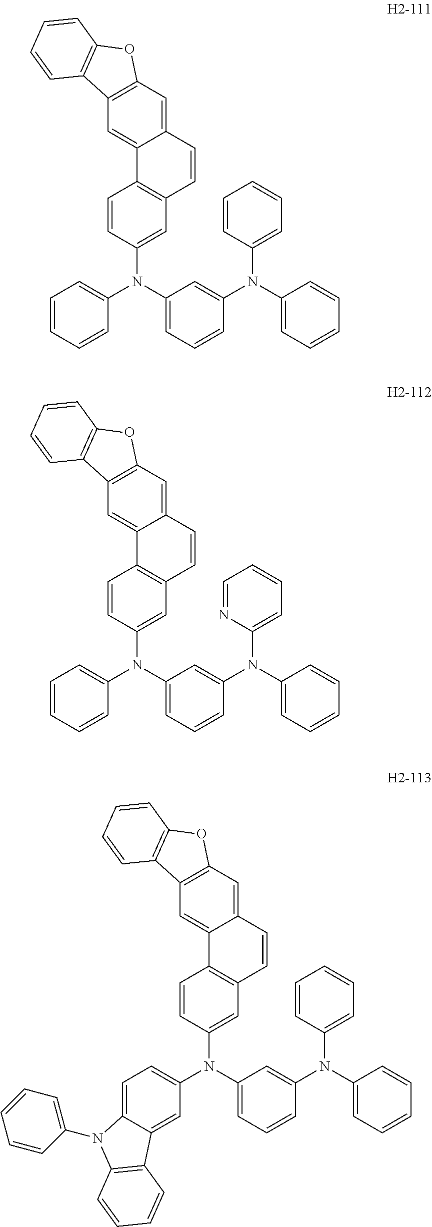 [SMILES: c1ccc(N(c2ccccc2)c2cccc(N(c3ccccc3)c3ccc4c(ccc5cc6oc7ccccc7c6cc54)c3)c2)cc1 ; c1ccc(N(c2cccc(N(c3ccccc3)c3ccccn3)c2)c2ccc3c(ccc4cc5oc6ccccc6c5cc43)c2)cc1 ; c1ccc(N(c2ccccc2)c2cccc(N(c3ccc4c(ccc5cc6oc7ccccc7c6cc54)c3)c3ccc4c(c3)c3ccccc3n4-c3ccccc3)c2)cc1] 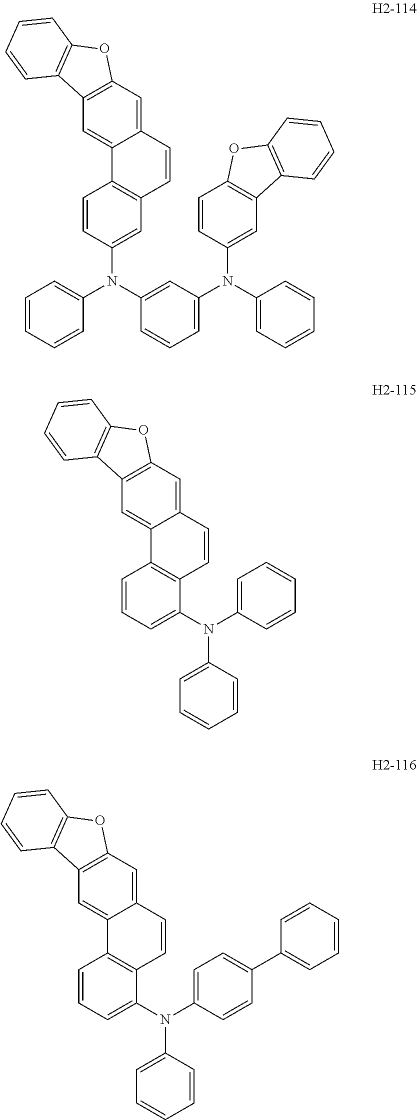 [SMILES: c1ccc(N(c2cccc(N(c3ccccc3)c3ccc4oc5ccccc5c4c3)c2)c2ccc3c(ccc4cc5oc6ccccc6c5cc43)c2)cc1 ; c1ccc(N(c2ccccc2)c2cccc3c2ccc2cc4oc5ccccc5c4cc23)cc1 ; c1ccc(-c2ccc(N(c3ccccc3)c3cccc4c3ccc3cc5oc6ccccc6c5cc34)cc2)cc1] 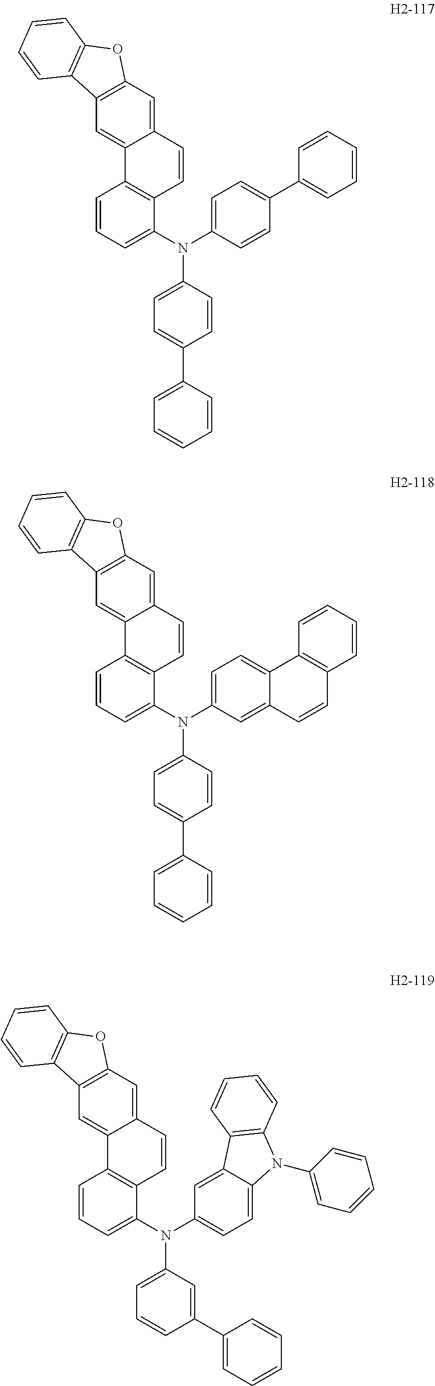 [SMILES: c1ccc(-c2ccc(N(c3ccc(-c4ccccc4)cc3)c3cccc4c3ccc3cc5oc6ccccc6c5cc34)cc2)cc1 ; c1ccc(-c2ccc(N(c3ccc4c(ccc5ccccc54)c3)c3cccc4c3ccc3cc5oc6ccccc6c5cc34)cc2)cc1 ; c1ccc(-c2cccc(N(c3ccc4c(c3)c3ccccc3n4-c3ccccc3)c3cccc4c3ccc3cc5oc6ccccc6c5cc34)c2)cc1] 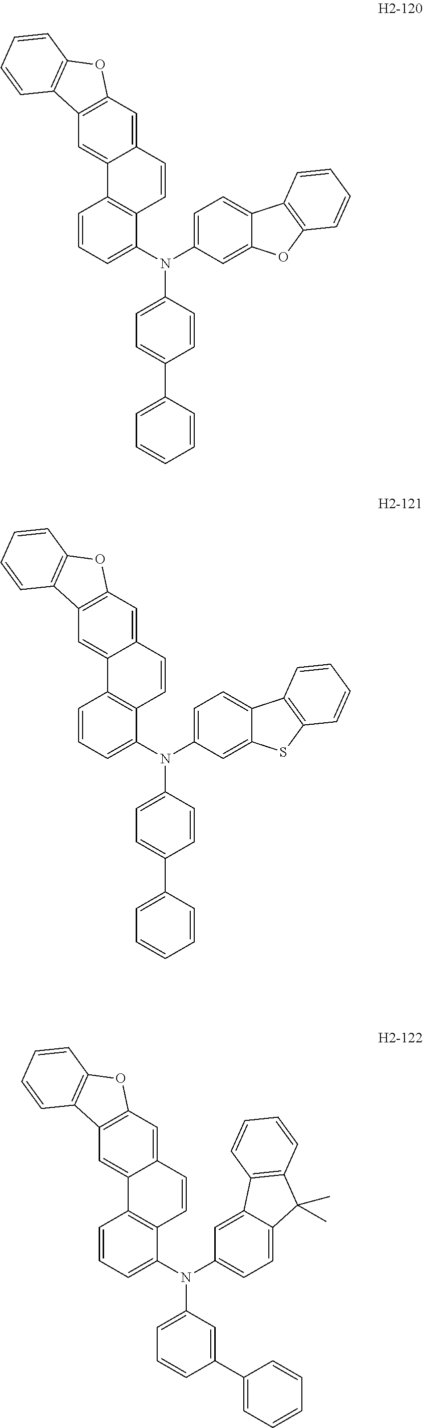 [SMILES: c1ccc(-c2ccc(N(c3ccc4c(c3)oc3ccccc34)c3cccc4c3ccc3cc5oc6ccccc6c5cc34)cc2)cc1 ; c1ccc(-c2ccc(N(c3ccc4c(c3)sc3ccccc34)c3cccc4c3ccc3cc5oc6ccccc6c5cc34)cc2)cc1 ; CC1(C)c2ccccc2-c2cc(N(c3cccc(-c4ccccc4)c3)c3cccc4c3ccc3cc5oc6ccccc6c5cc34)ccc21] 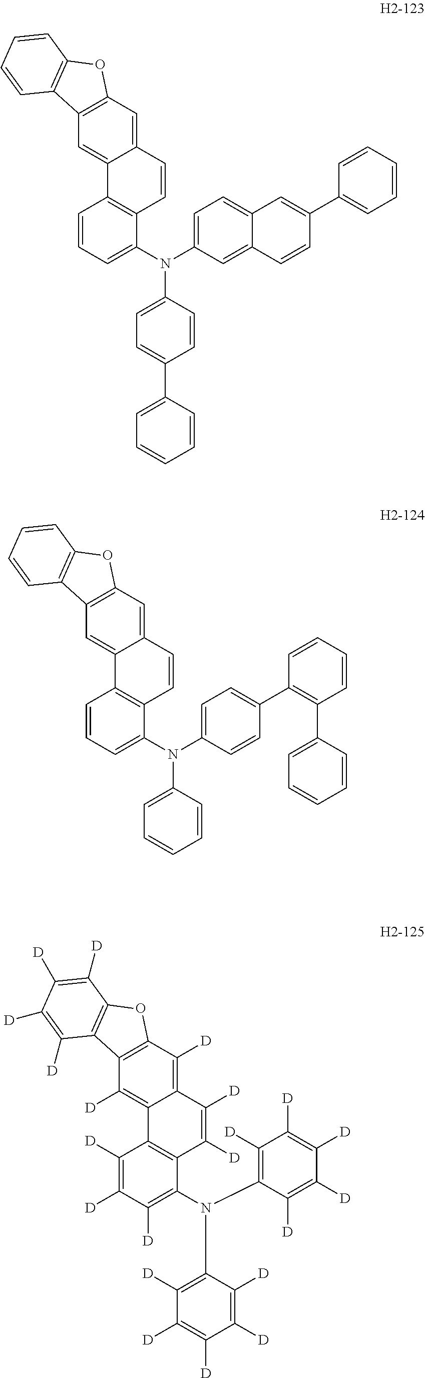 [SMILES: c1ccc(-c2ccc(N(c3ccc4cc(-c5ccccc5)ccc4c3)c3cccc4c3ccc3cc5oc6ccccc6c5cc34)cc2)cc1 ; c1ccc(-c2ccccc2-c2ccc(N(c3ccccc3)c3cccc4c3ccc3cc5oc6ccccc6c5cc34)cc2)cc1 ; [2H]c1c([2H])c([2H])c(N(c2c([2H])c([2H])c([2H])c([2H])c2[2H])c2c([2H])c([2H])c([2H])c3c2c([2H])c([2H])c2c([2H])c4oc5c([2H])c([2H])c([2H])c([2H])c5c4c([2H])c23)c([2H])c1[2H]] 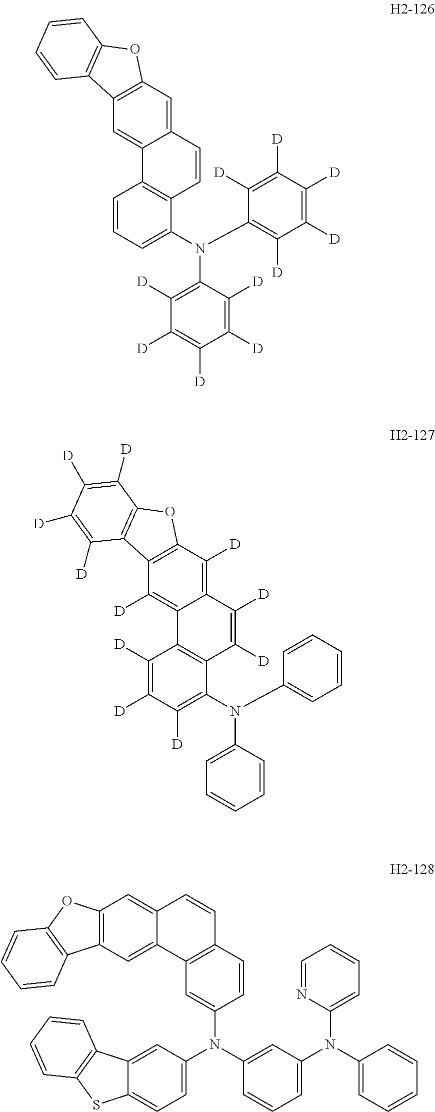 [SMILES: [2H]c1c([2H])c([2H])c(N(c2c([2H])c([2H])c([2H])c([2H])c2[2H])c2cccc3c2ccc2cc4oc5ccccc5c4cc23)c([2H])c1[2H] ; [2H]c1c([2H])c([2H])c2c(oc3c([2H])c4c([2H])c([2H])c5c(N(c6ccccc6)c6ccccc6)c([2H])c([2H])c([2H])c5c4c([2H])c32)c1[2H] ; c1ccc(N(c2cccc(N(c3ccc4ccc5cc6oc7ccccc7c6cc5c4c3)c3ccc4sc5ccccc5c4c3)c2)c2ccccn2)cc1] 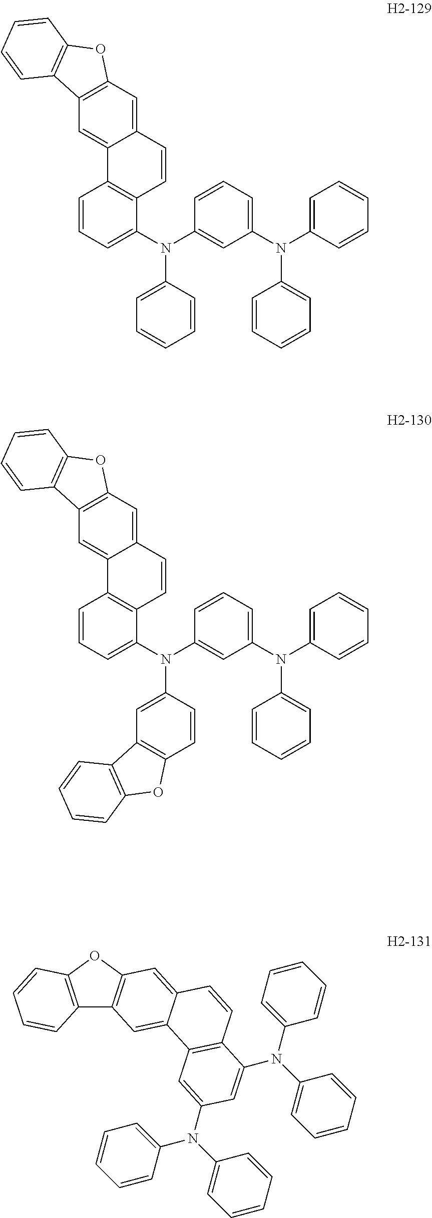 [SMILES: c1ccc(N(c2ccccc2)c2cccc(N(c3ccccc3)c3cccc4c3ccc3cc5oc6ccccc6c5cc34)c2)cc1 ; c1ccc(N(c2ccccc2)c2cccc(N(c3ccc4oc5ccccc5c4c3)c3cccc4c3ccc3cc5oc6ccccc6c5cc34)c2)cc1 ; c1ccc(N(c2ccccc2)c2cc(N(c3ccccc3)c3ccccc3)c3ccc4cc5oc6ccccc6c5cc4c3c2)cc1] 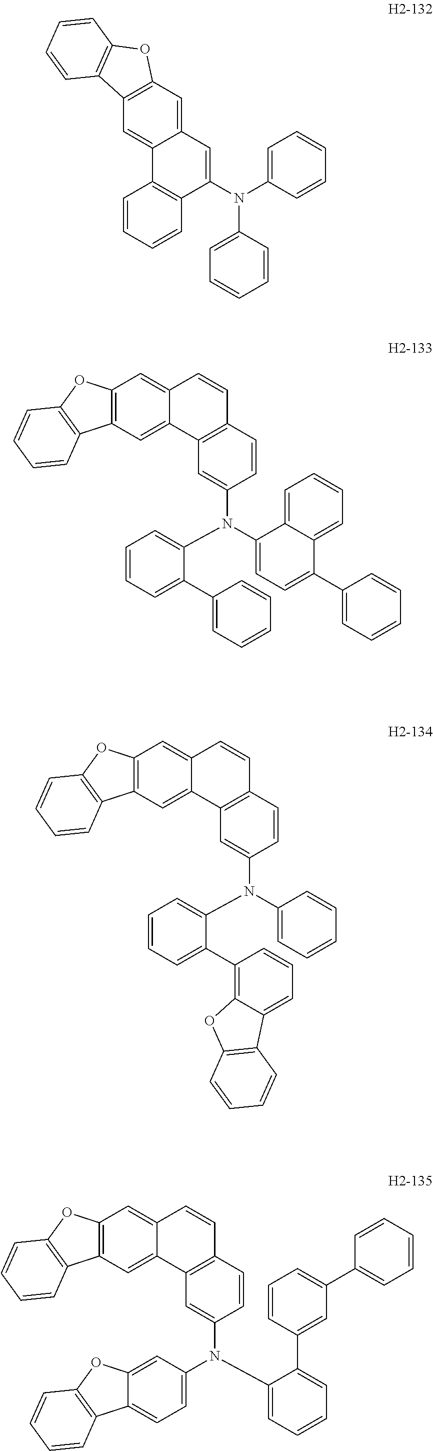 [SMILES: c1ccc(N(c2ccccc2)c2cc3cc4oc5ccccc5c4cc3c3ccccc23)cc1 ; c1ccc(-c2ccccc2N(c2ccc3ccc4cc5oc6ccccc6c5cc4c3c2)c2ccc(-c3ccccc3)c3ccccc23)cc1 ; c1ccc(N(c2ccc3ccc4cc5oc6ccccc6c5cc4c3c2)c2ccccc2-c2cccc3c2oc2ccccc23)cc1 ; c1ccc(-c2cccc(-c3ccccc3N(c3ccc4c(c3)oc3ccccc34)c3ccc4ccc5cc6oc7ccccc7c6cc5c4c3)c2)cc1] 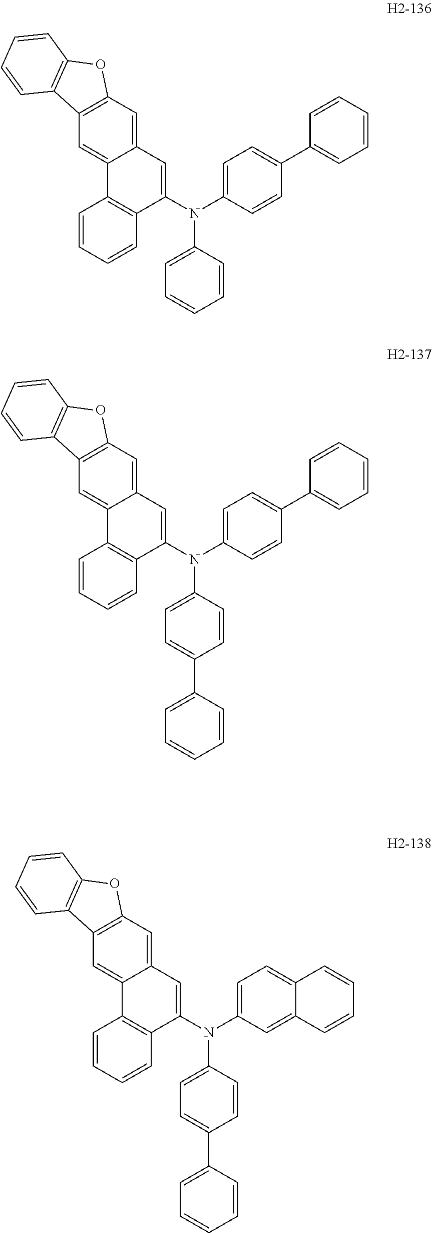 [SMILES: c1ccc(-c2ccc(N(c3ccccc3)c3cc4cc5oc6ccccc6c5cc4c4ccccc34)cc2)cc1 ; c1ccc(-c2ccc(N(c3ccc(-c4ccccc4)cc3)c3cc4cc5oc6ccccc6c5cc4c4ccccc34)cc2)cc1 ; c1ccc(-c2ccc(N(c3ccc4ccccc4c3)c3cc4cc5oc6ccccc6c5cc4c4ccccc34)cc2)cc1] 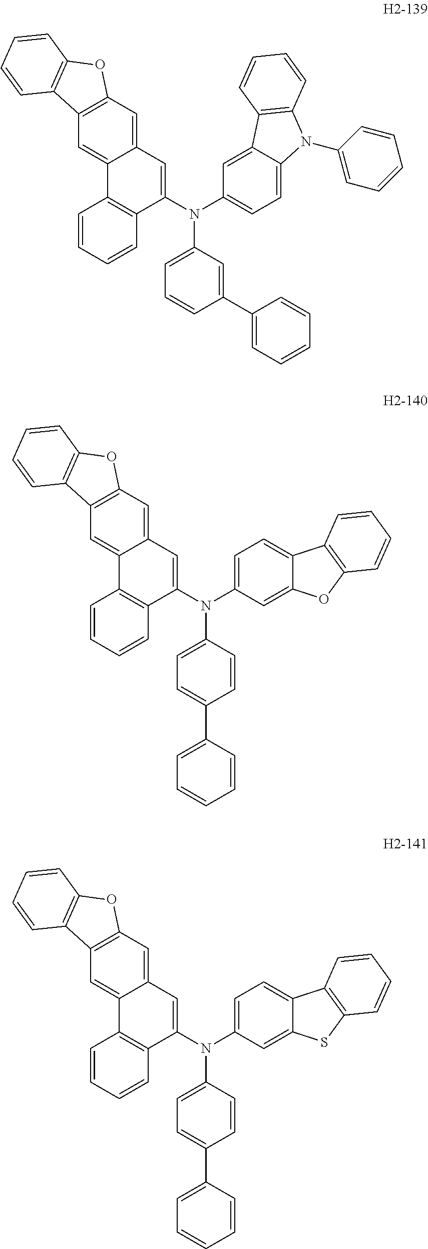 [SMILES: c1ccc(-c2cccc(N(c3ccc4c(c3)c3ccccc3n4-c3ccccc3)c3cc4cc5oc6ccccc6c5cc4c4ccccc34)c2)cc1 ; c1ccc(-c2ccc(N(c3ccc4c(c3)oc3ccccc34)c3cc4cc5oc6ccccc6c5cc4c4ccccc34)cc2)cc1 ; c1ccc(-c2ccc(N(c3ccc4c(c3)sc3ccccc34)c3cc4cc5oc6ccccc6c5cc4c4ccccc34)cc2)cc1] 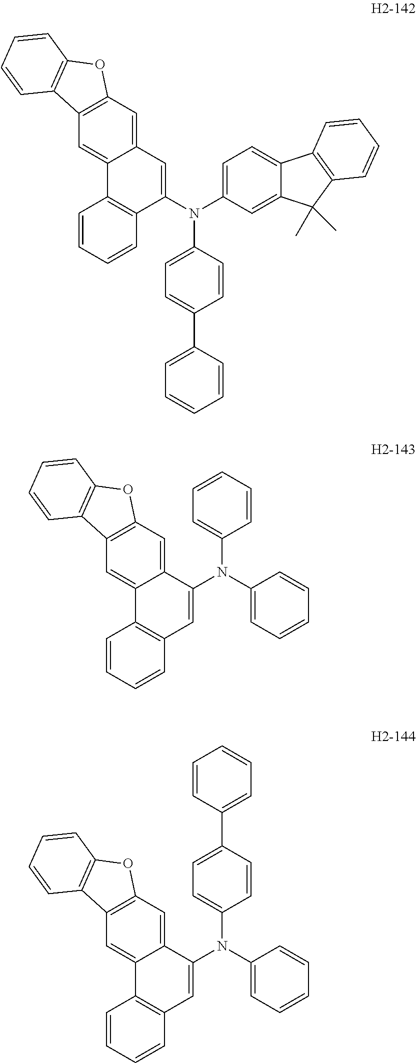 [SMILES: CC1(C)c2ccccc2-c2ccc(N(c3ccc(-c4ccccc4)cc3)c3cc4cc5oc6ccccc6c5cc4c4ccccc34)cc21 ; c1ccc(N(c2ccccc2)c2cc3ccccc3c3cc4c(cc23)oc2ccccc24)cc1 ; c1ccc(-c2ccc(N(c3ccccc3)c3cc4ccccc4c4cc5c(cc34)oc3ccccc35)cc2)cc1] 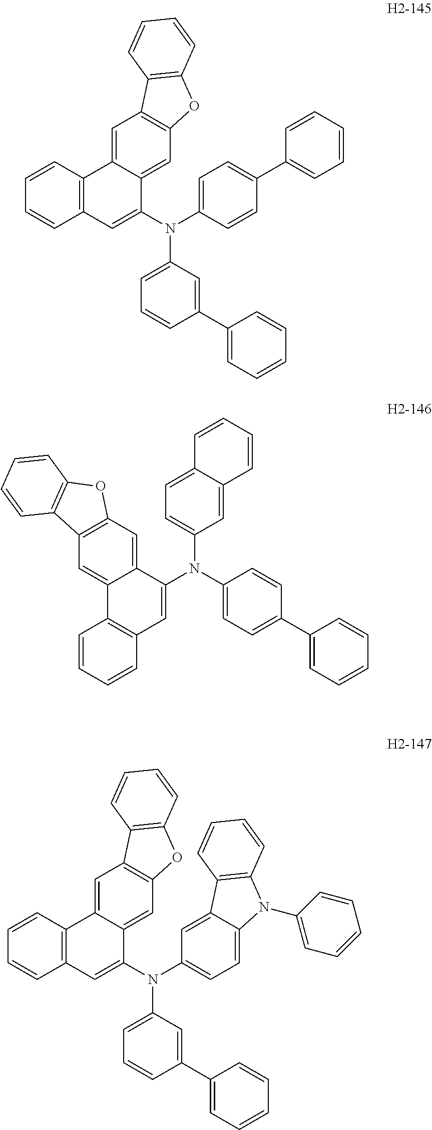 [SMILES: c1ccc(-c2ccc(N(c3cccc(-c4ccccc4)c3)c3cc4ccccc4c4cc5c(cc34)oc3ccccc35)cc2)cc1 ; c1ccc(-c2ccc(N(c3ccc4ccccc4c3)c3cc4ccccc4c4cc5c(cc34)oc3ccccc35)cc2)cc1 ; c1ccc(-c2cccc(N(c3ccc4c(c3)c3ccccc3n4-c3ccccc3)c3cc4ccccc4c4cc5c(cc34)oc3ccccc35)c2)cc1] 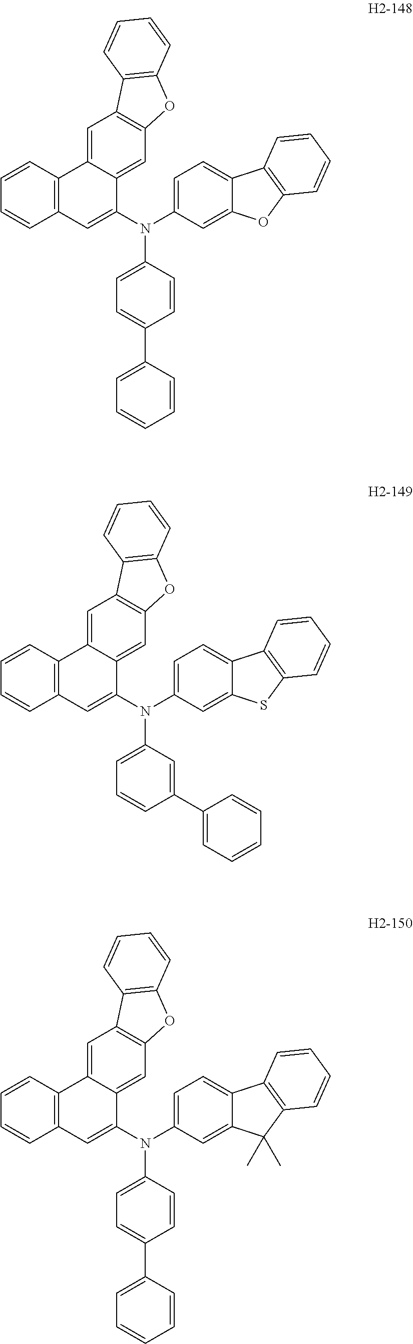 [SMILES: c1ccc(-c2ccc(N(c3ccc4c(c3)oc3ccccc34)c3cc4ccccc4c4cc5c(cc34)oc3ccccc35)cc2)cc1 ; c1ccc(-c2cccc(N(c3ccc4c(c3)sc3ccccc34)c3cc4ccccc4c4cc5c(cc34)oc3ccccc35)c2)cc1 ; CC1(C)c2ccccc2-c2ccc(N(c3ccc(-c4ccccc4)cc3)c3cc4ccccc4c4cc5c(cc34)oc3ccccc35)cc21] 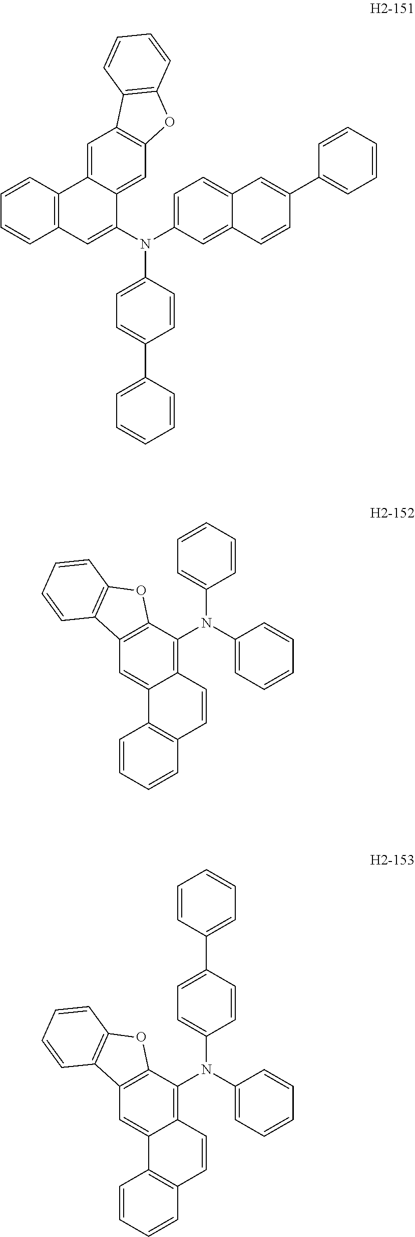 [SMILES: c1ccc(-c2ccc(N(c3ccc4cc(-c5ccccc5)ccc4c3)c3cc4ccccc4c4cc5c(cc34)oc3ccccc35)cc2)cc1 ; c1ccc(N(c2ccccc2)c2c3ccc4ccccc4c3cc3c2oc2ccccc23)cc1 ; c1ccc(-c2ccc(N(c3ccccc3)c3c4ccc5ccccc5c4cc4c3oc3ccccc34)cc2)cc1] 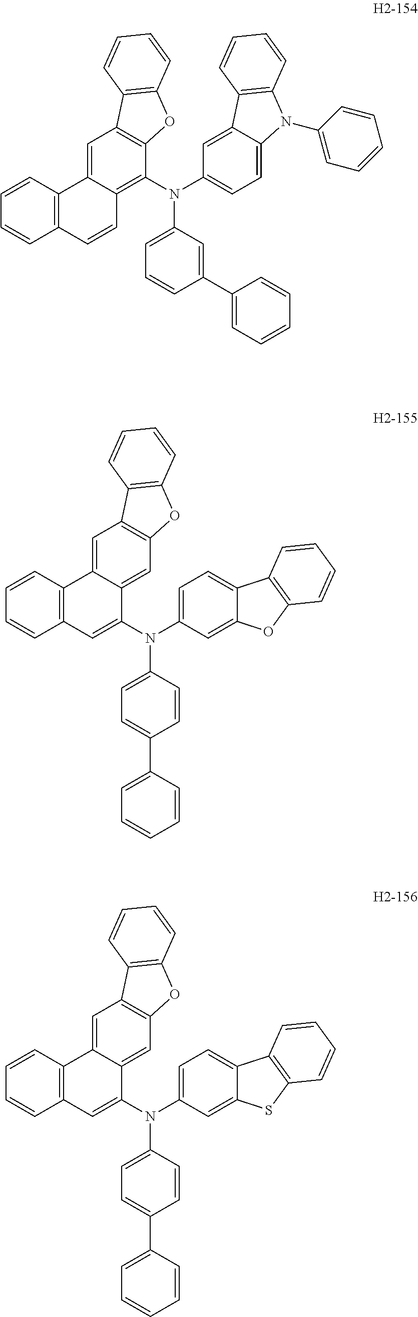 [SMILES: c1ccc(-c2cccc(N(c3ccc4c(c3)c3ccccc3n4-c3ccccc3)c3c4ccc5ccccc5c4cc4c3oc3ccccc34)c2)cc1 ; c1ccc(-c2ccc(N(c3ccc4c(c3)oc3ccccc34)c3cc4ccccc4c4cc5c(cc34)oc3ccccc35)cc2)cc1 ; c1ccc(-c2ccc(N(c3ccc4c(c3)sc3ccccc34)c3cc4ccccc4c4cc5c(cc34)oc3ccccc35)cc2)cc1] 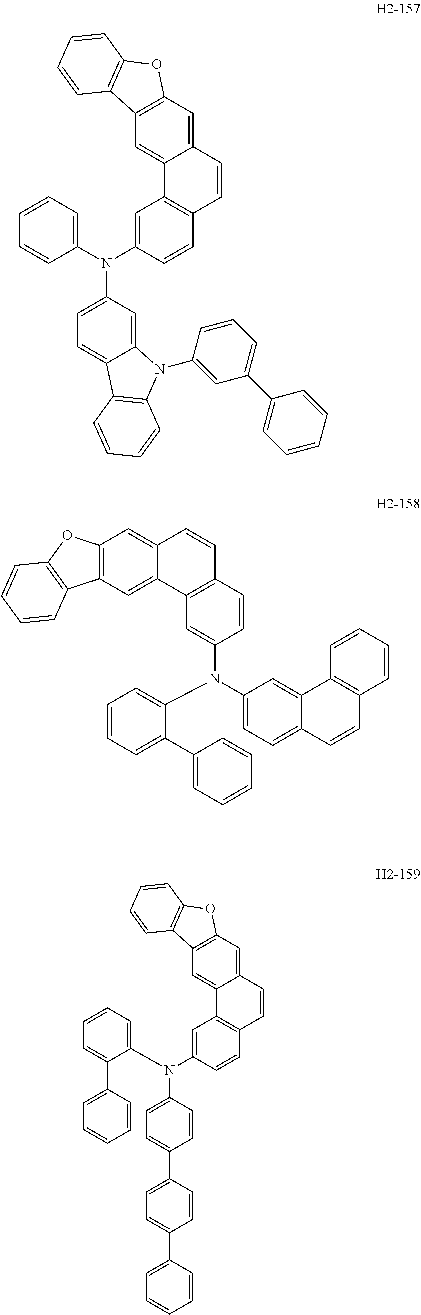 [SMILES: c1ccc(-c2cccc(-n3c4ccccc4c4ccc(N(c5ccccc5)c5ccc6ccc7cc8oc9ccccc9c8cc7c6c5)cc43)c2)cc1 ; c1ccc(-c2ccccc2N(c2ccc3ccc4ccccc4c3c2)c2ccc3ccc4cc5oc6ccccc6c5cc4c3c2)cc1 ; c1ccc(-c2ccc(-c3ccc(N(c4ccc5ccc6cc7oc8ccccc8c7cc6c5c4)c4ccccc4-c4ccccc4)cc3)cc2)cc1] 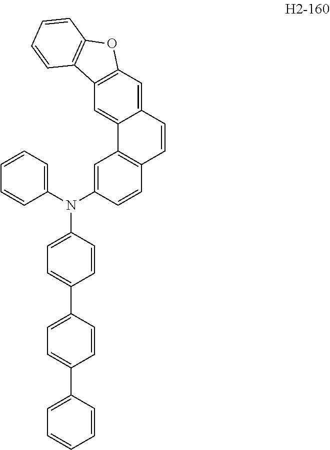 [SMILES: c1ccc(-c2ccc(-c3ccc(N(c4ccccc4)c4ccc5ccc6cc7oc8ccccc8c7cc6c5c4)cc3)cc2)cc1] 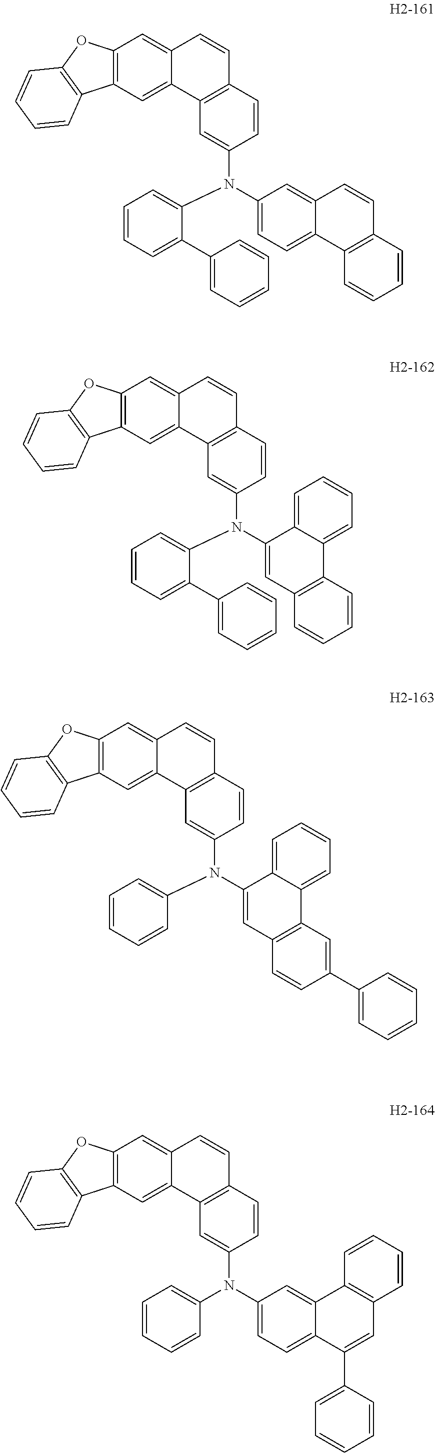 [SMILES: c1ccc(-c2ccccc2N(c2ccc3c(ccc4ccccc43)c2)c2ccc3ccc4cc5oc6ccccc6c5cc4c3c2)cc1 ; c1ccc(-c2ccccc2N(c2ccc3ccc4cc5oc6ccccc6c5cc4c3c2)c2cc3ccccc3c3ccccc23)cc1 ; c1ccc(-c2ccc3cc(N(c4ccccc4)c4ccc5ccc6cc7oc8ccccc8c7cc6c5c4)c4ccccc4c3c2)cc1 ; c1ccc(-c2cc3ccccc3c3cc(N(c4ccccc4)c4ccc5ccc6cc7oc8ccccc8c7cc6c5c4)ccc23)cc1] 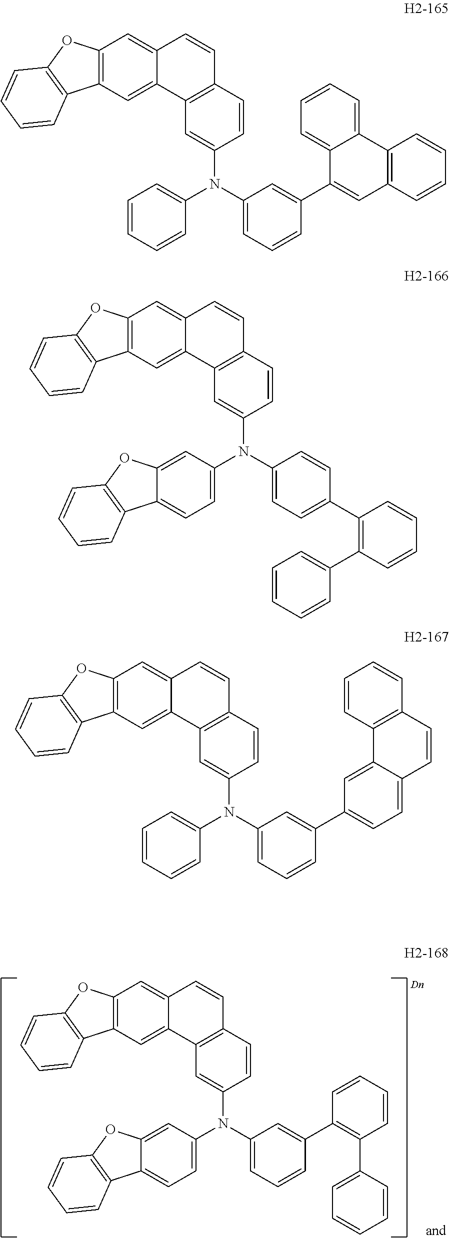 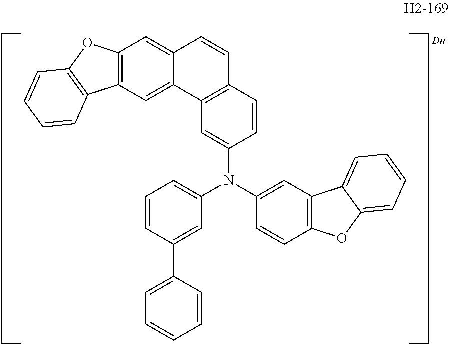
wherein in the compounds above, Dₙ means that n number of hydrogens are replaced with deuterium, and n represents an integer from 1 to the maximum number of hydrogens in the compound.

17. The organic electroluminescent device according to claim 12, wherein the compound represented by Formula 7 is selected from the following compounds: 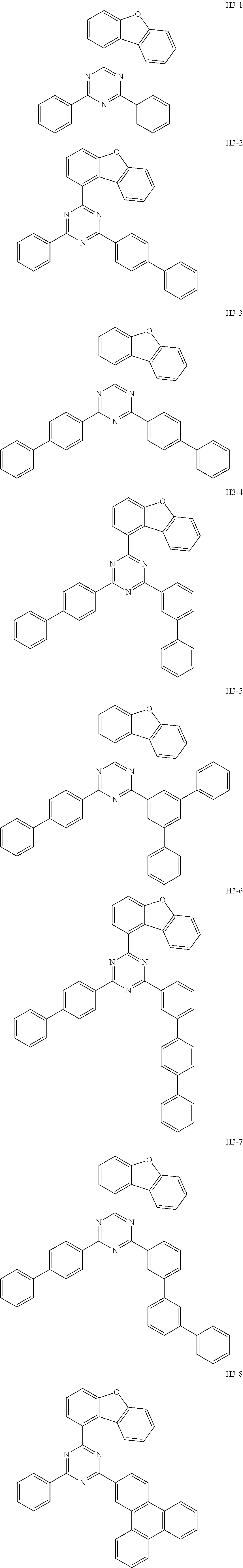 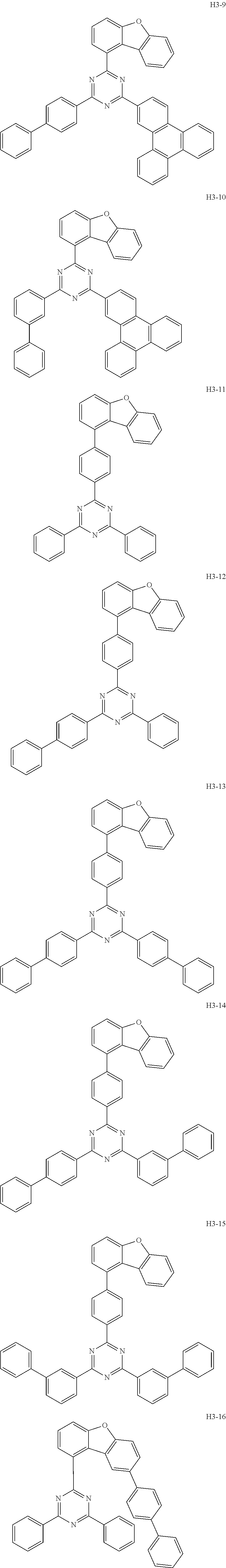 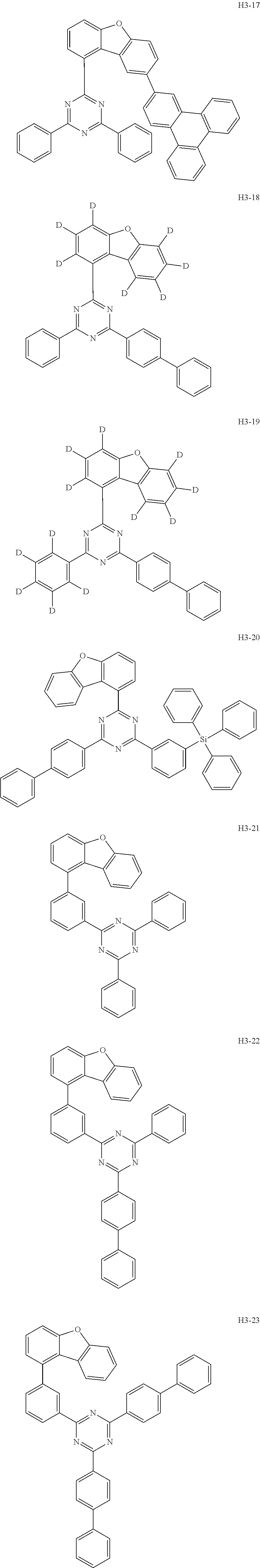 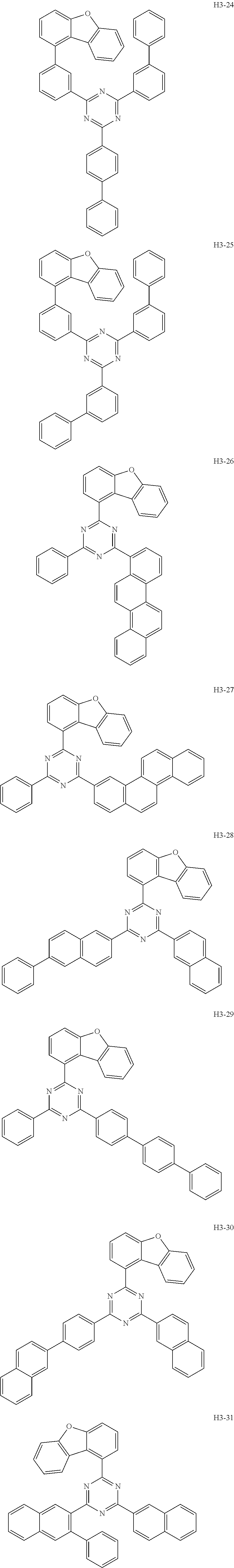 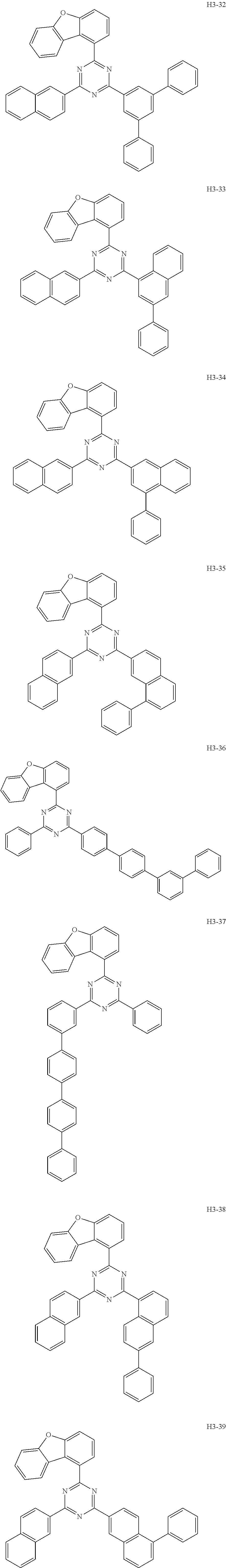 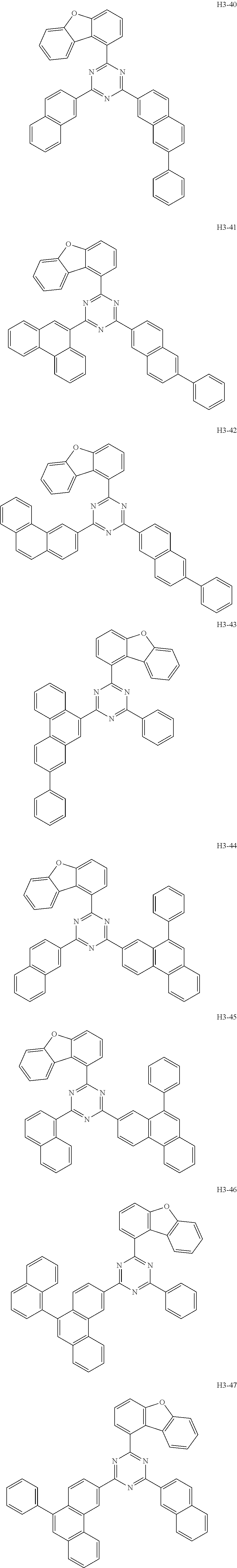 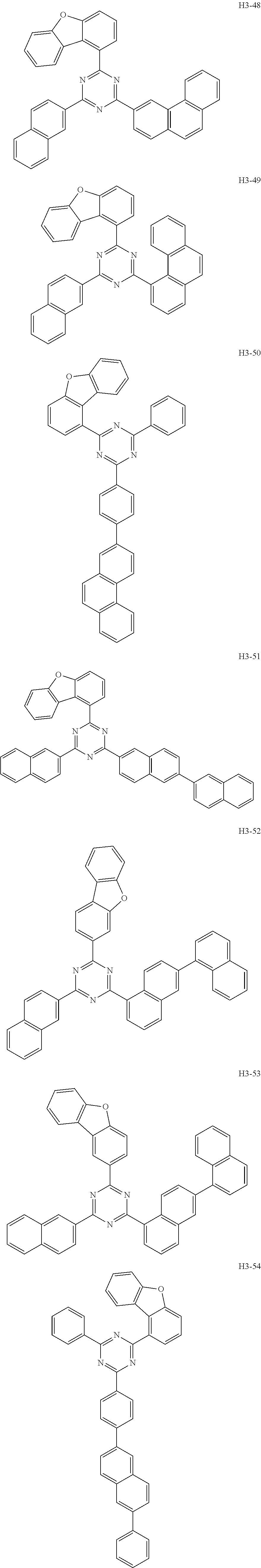 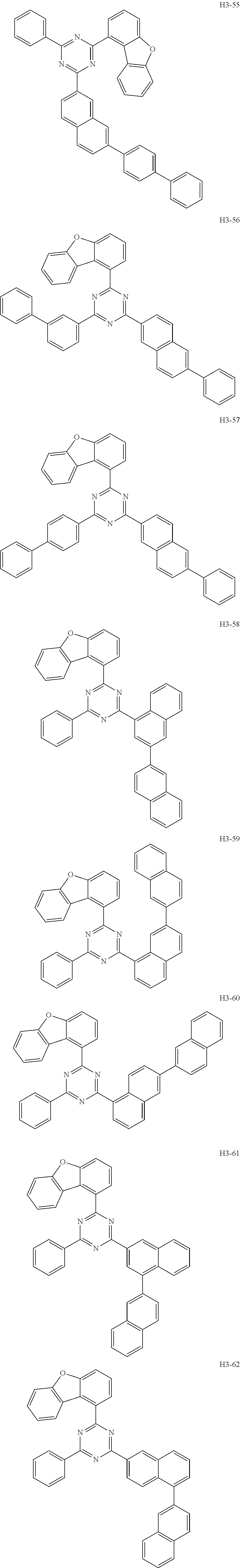 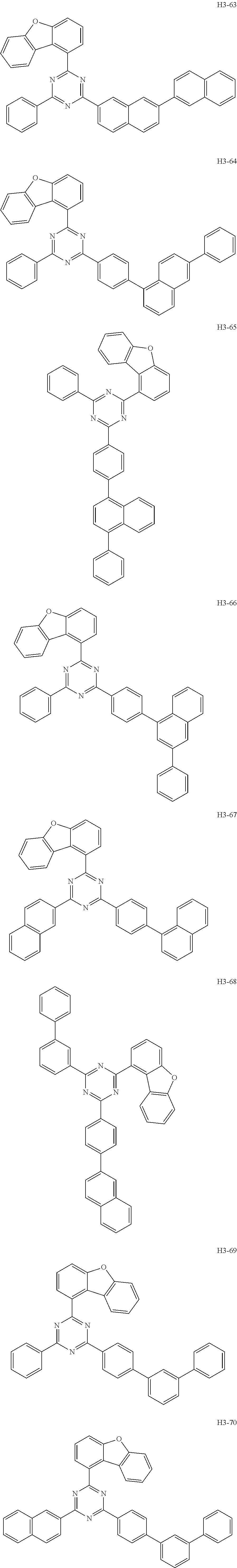 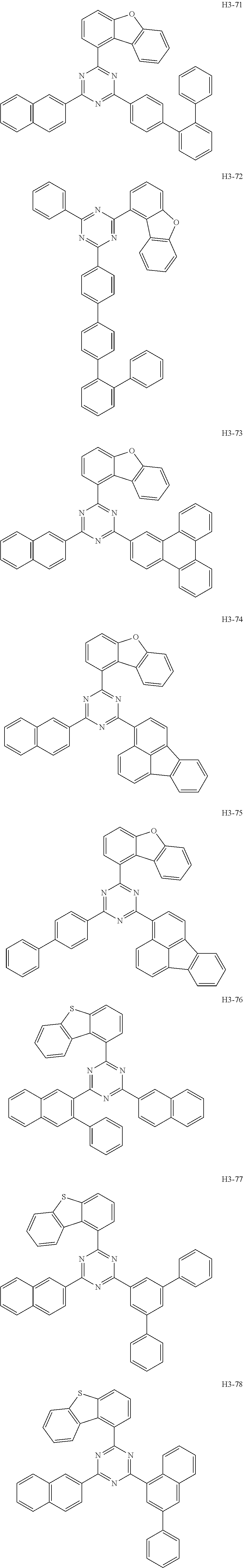 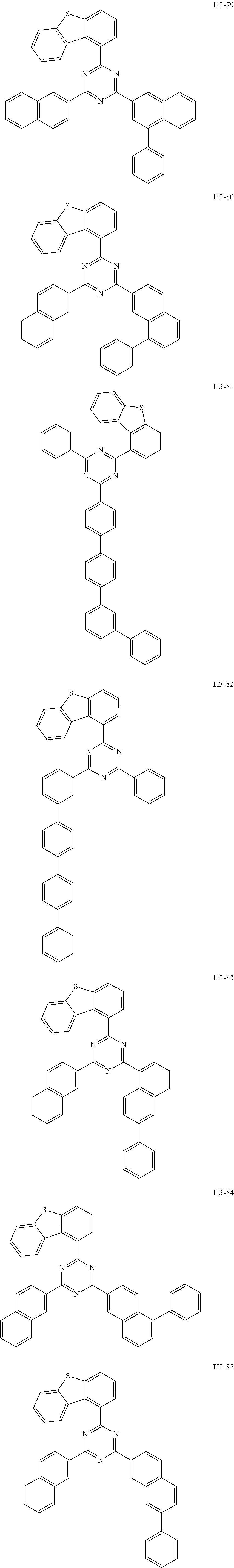 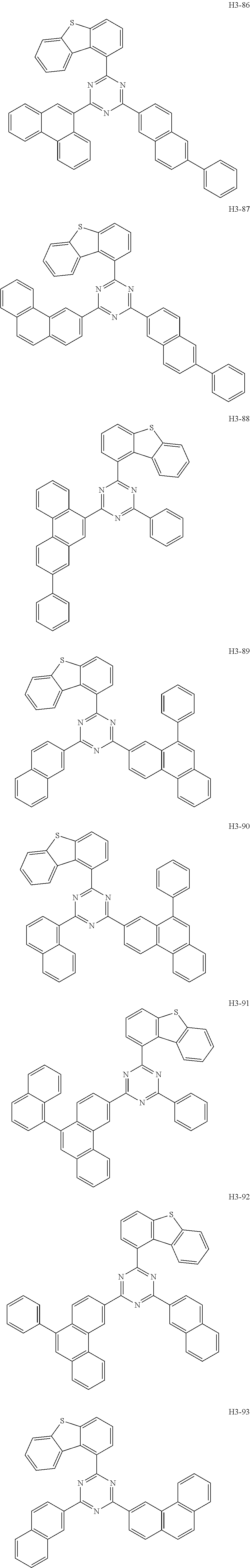 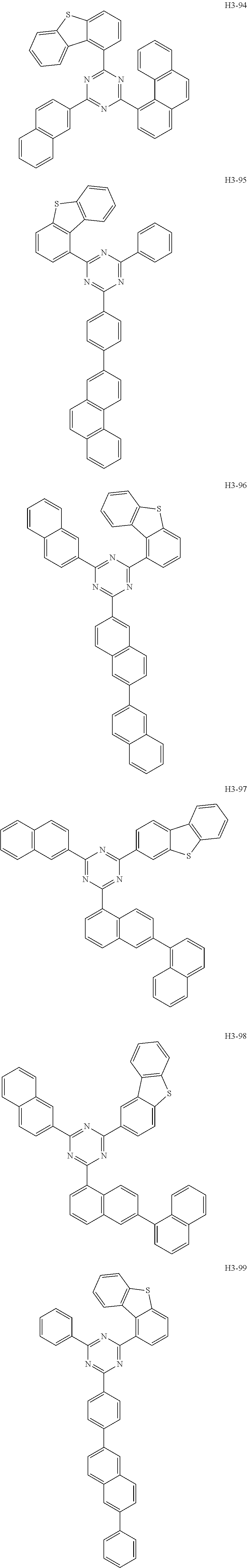 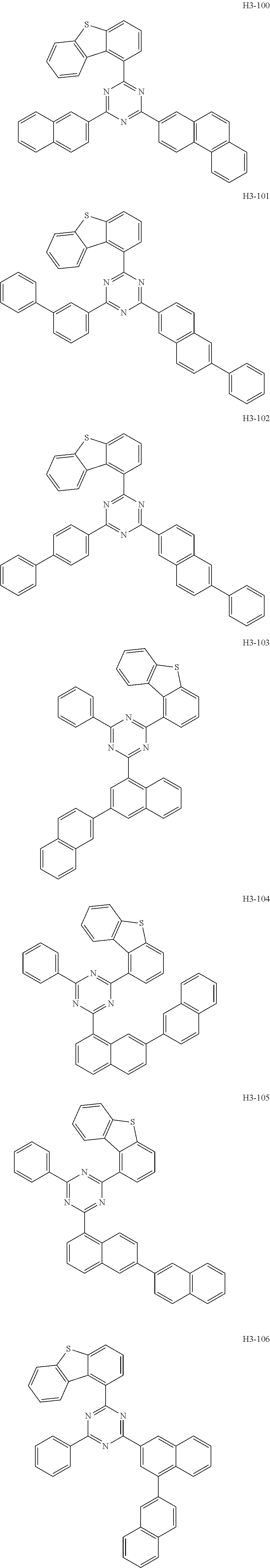 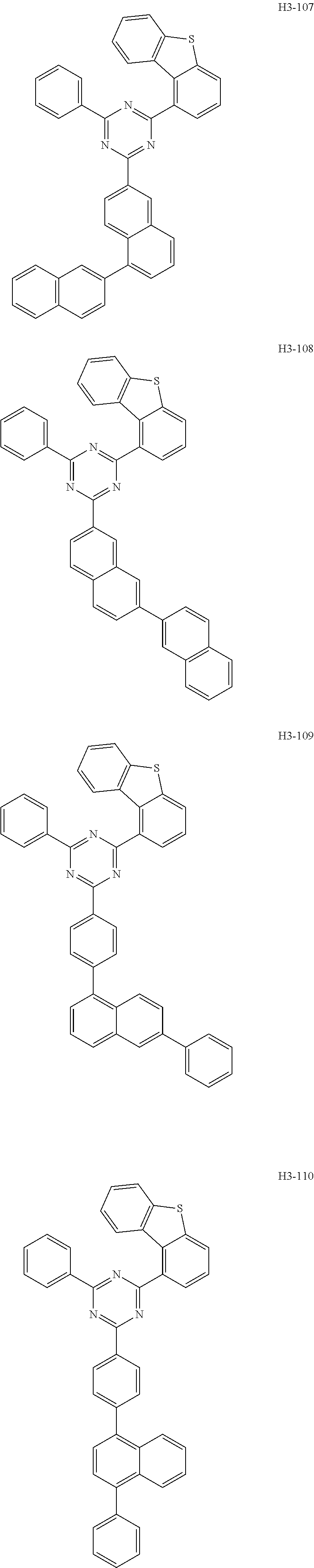 [SMILES: c1ccc(-c2nc(-c3ccc4c(-c5ccc6ccccc6c5)cccc4c3)nc(-c3cccc4sc5ccccc5c34)n2)cc1 ; c1ccc(-c2nc(-c3ccc4ccc(-c5ccc6ccccc6c5)cc4c3)nc(-c3cccc4sc5ccccc5c34)n2)cc1 ; c1ccc(-c2ccc3c(-c4ccc(-c5nc(-c6ccccc6)nc(-c6cccc7sc8ccccc8c67)n5)cc4)cccc3c2)cc1 ; c1ccc(-c2nc(-c3ccc(-c4ccc(-c5ccccc5)c5ccccc45)cc3)nc(-c3cccc4sc5ccccc5c34)n2)cc1] 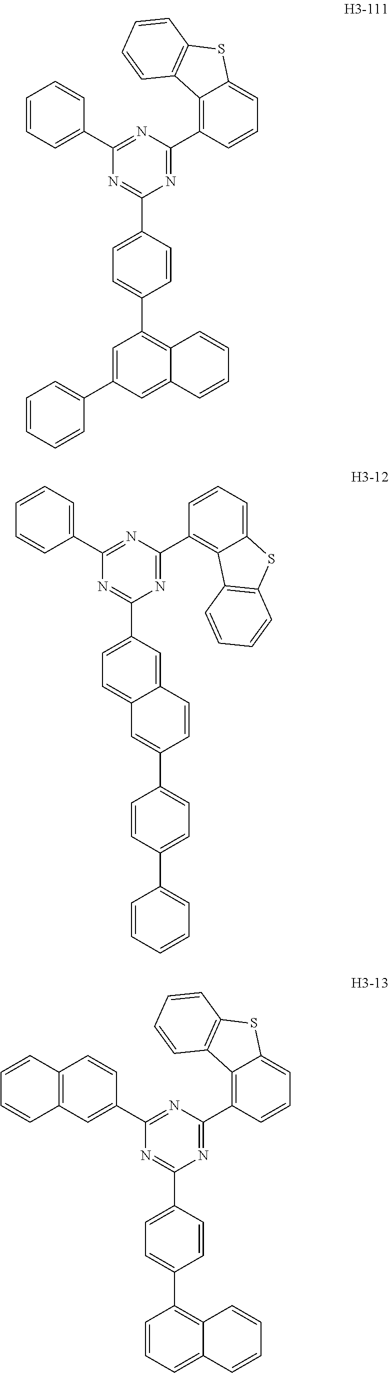 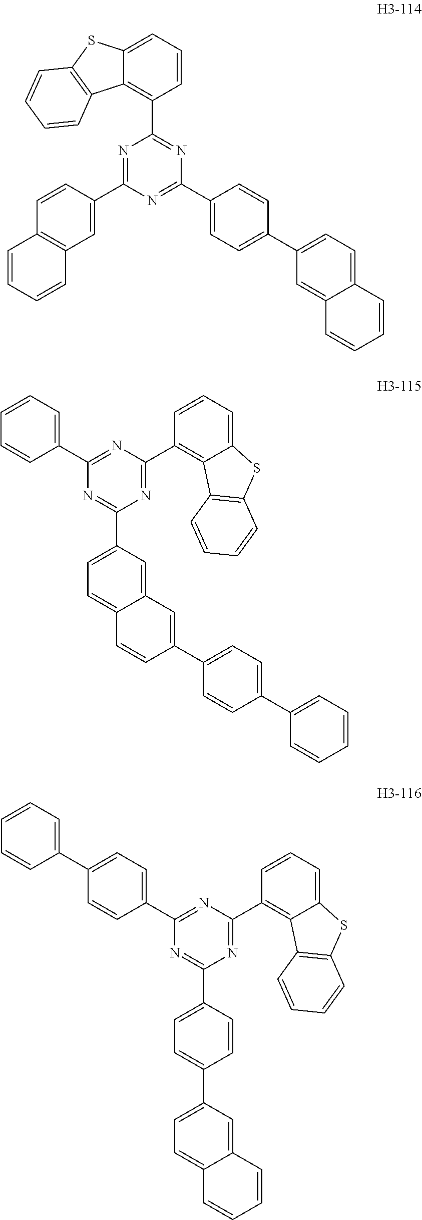 [SMILES: c1ccc2cc(-c3ccc(-c4nc(-c5ccc6ccccc6c5)nc(-c5cccc6sc7ccccc7c56)n4)cc3)ccc2c1 ; c1ccc(-c2ccc(-c3ccc4ccc(-c5nc(-c6ccccc6)nc(-c6cccc7sc8ccccc8c67)n5)cc4c3)cc2)cc1 ; c1ccc(-c2ccc(-c3nc(-c4ccc(-c5ccc6ccccc6c5)cc4)nc(-c4cccc5sc6ccccc6c45)n3)cc2)cc1] 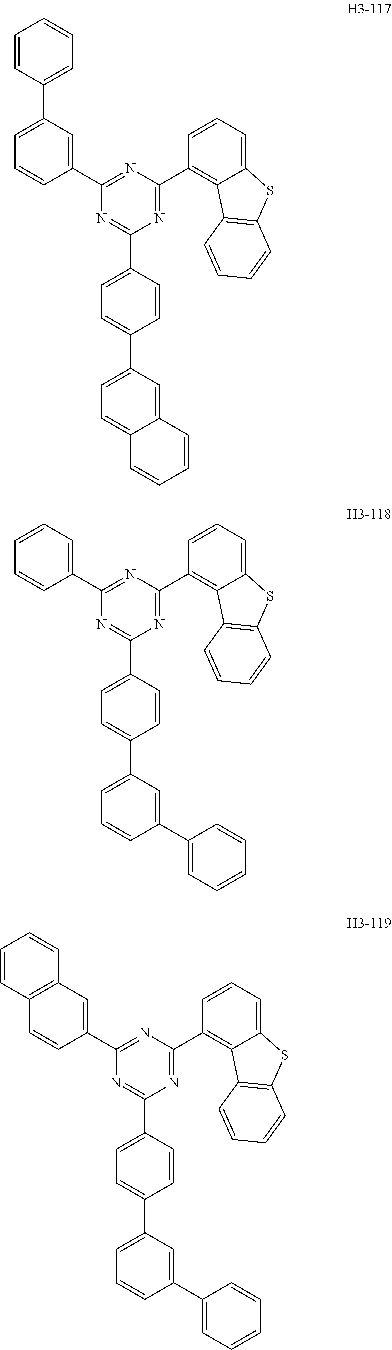 [SMILES: c1ccc(-c2cccc(-c3nc(-c4ccc(-c5ccc6ccccc6c5)cc4)nc(-c4cccc5sc6ccccc6c45)n3)c2)cc1 ; c1ccc(-c2cccc(-c3ccc(-c4nc(-c5ccccc5)nc(-c5cccc6sc7ccccc7c56)n4)cc3)c2)cc1 ; c1ccc(-c2cccc(-c3ccc(-c4nc(-c5ccc6ccccc6c5)nc(-c5cccc6sc7ccccc7c56)n4)cc3)c2)cc1] 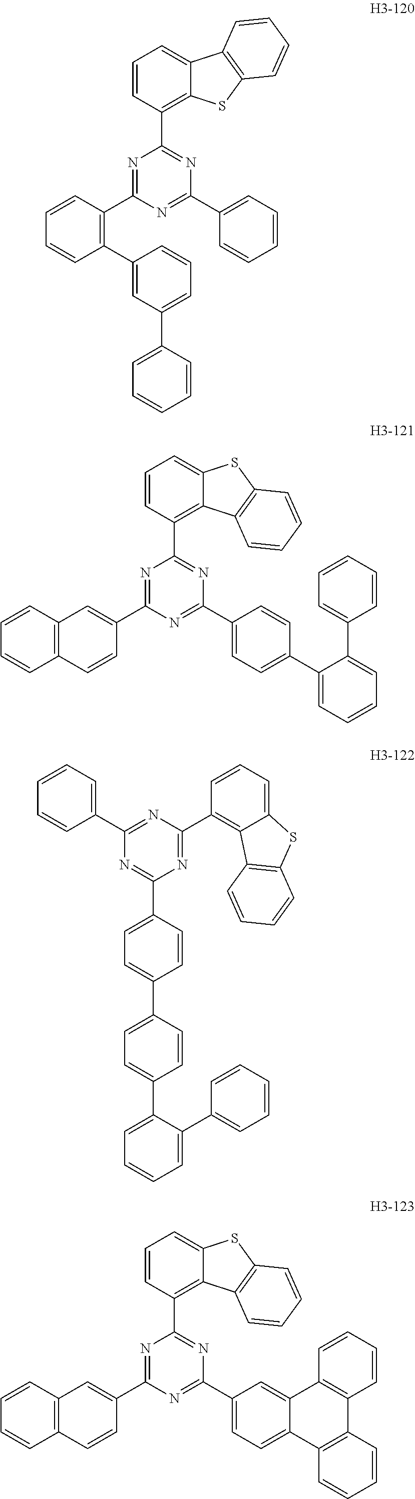 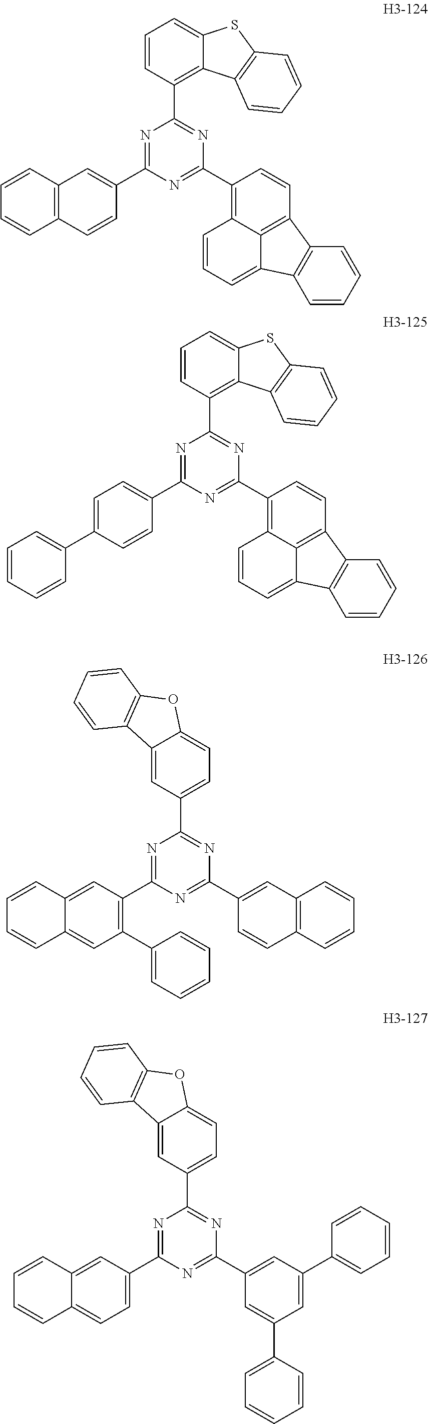 [SMILES: c1ccc2c(c1)-c1cccc3c(-c4nc(-c5ccc6ccccc6c5)nc(-c5cccc6sc7ccccc7c56)n4)ccc-2c13 ; c1ccc(-c2ccc(-c3nc(-c4ccc5c6c(cccc46)-c4ccccc4-5)nc(-c4cccc5sc6ccccc6c45)n3)cc2)cc1 ; c1ccc(-c2cc3ccccc3cc2-c2nc(-c3ccc4ccccc4c3)nc(-c3ccc4oc5ccccc5c4c3)n2)cc1 ; c1ccc(-c2cc(-c3ccccc3)cc(-c3nc(-c4ccc5ccccc5c4)nc(-c4ccc5oc6ccccc6c5c4)n3)c2)cc1] 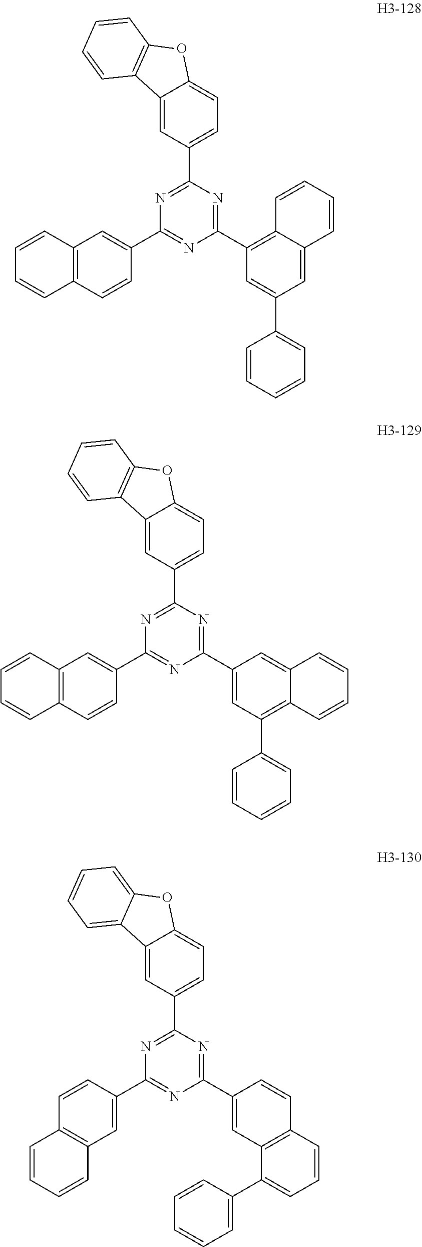 [SMILES: c1ccc(-c2cc(-c3nc(-c4ccc5ccccc5c4)nc(-c4ccc5oc6ccccc6c5c4)n3)c3ccccc3c2)cc1 ; c1ccc(-c2cc(-c3nc(-c4ccc5ccccc5c4)nc(-c4ccc5oc6ccccc6c5c4)n3)cc3ccccc23)cc1 ; c1ccc(-c2cccc3ccc(-c4nc(-c5ccc6ccccc6c5)nc(-c5ccc6oc7ccccc7c6c5)n4)cc23)cc1] 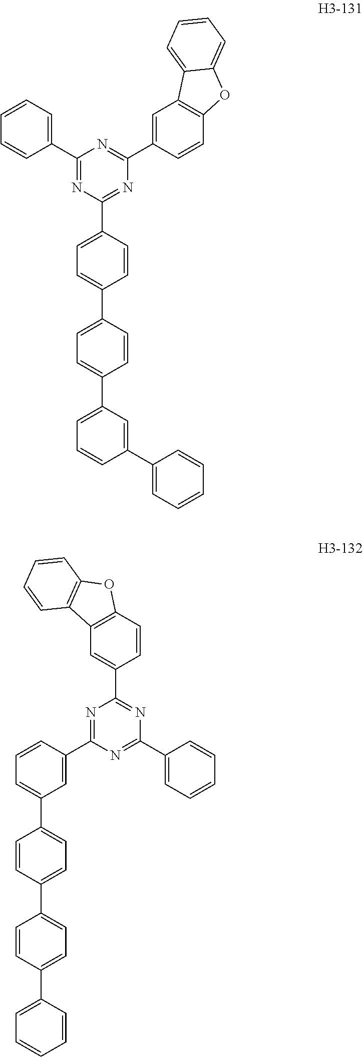 [SMILES: c1ccc(-c2cccc(-c3ccc(-c4ccc(-c5nc(-c6ccccc6)nc(-c6ccc7oc8ccccc8c7c6)n5)cc4)cc3)c2)cc1 ; c1ccc(-c2ccc(-c3ccc(-c4cccc(-c5nc(-c6ccccc6)nc(-c6ccc7oc8ccccc8c7c6)n5)c4)cc3)cc2)cc1] 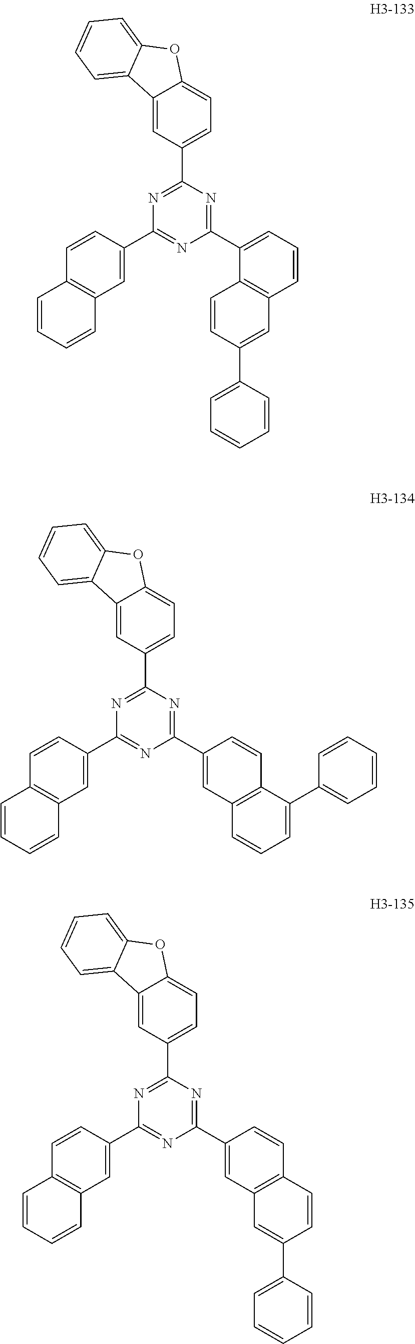 [SMILES: c1ccc(-c2ccc3c(-c4nc(-c5ccc6ccccc6c5)nc(-c5ccc6oc7ccccc7c6c5)n4)cccc3c2)cc1 ; c1ccc(-c2cccc3cc(-c4nc(-c5ccc6ccccc6c5)nc(-c5ccc6oc7ccccc7c6c5)n4)ccc23)cc1 ; c1ccc(-c2ccc3ccc(-c4nc(-c5ccc6ccccc6c5)nc(-c5ccc6oc7ccccc7c6c5)n4)cc3c2)cc1] 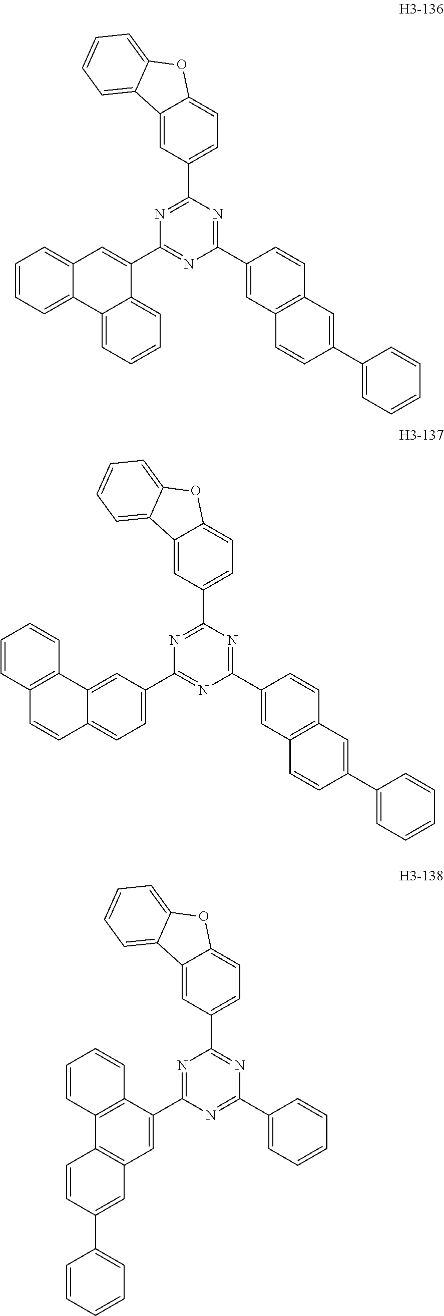 [SMILES: c1ccc(-c2ccc3cc(-c4nc(-c5ccc6oc7ccccc7c6c5)nc(-c5cc6ccccc6c6ccccc56)n4)ccc3c2)cc1 ; c1ccc(-c2ccc3cc(-c4nc(-c5ccc6ccc7ccccc7c6c5)nc(-c5ccc6oc7ccccc7c6c5)n4)ccc3c2)cc1 ; c1ccc(-c2ccc3c(c2)cc(-c2nc(-c4ccccc4)nc(-c4ccc5oc6ccccc6c5c4)n2)c2ccccc23)cc1] 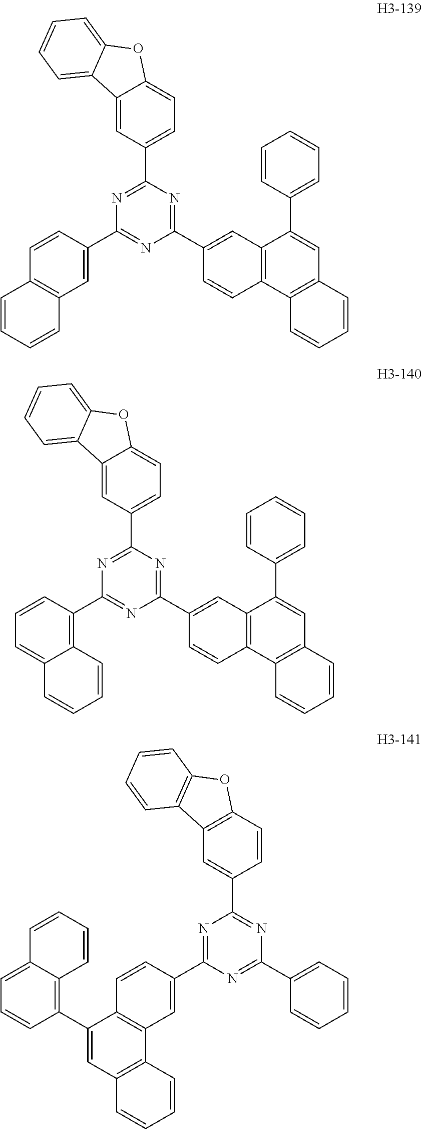 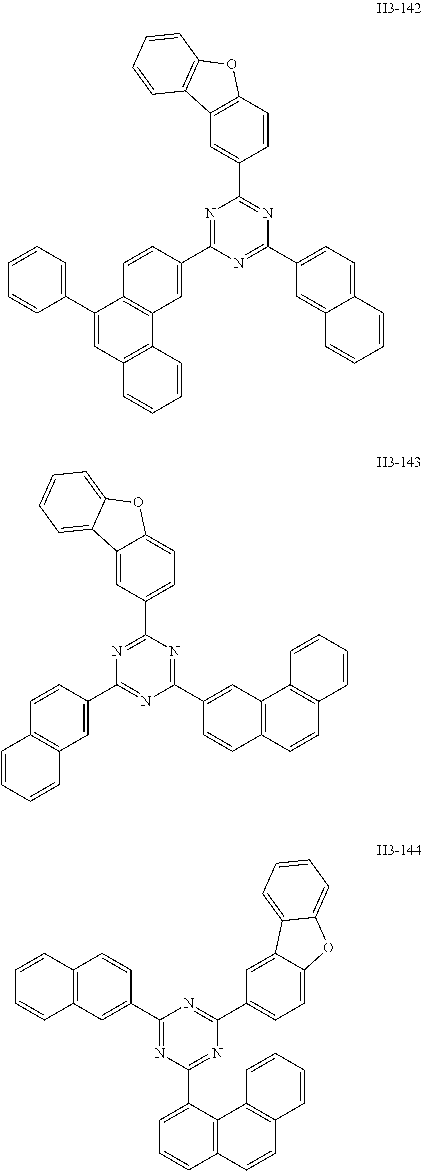 [SMILES: c1ccc(-c2cc3ccccc3c3cc(-c4nc(-c5ccc6ccccc6c5)nc(-c5ccc6oc7ccccc7c6c5)n4)ccc23)cc1 ; c1ccc2cc(-c3nc(-c4ccc5ccc6ccccc6c5c4)nc(-c4ccc5oc6ccccc6c5c4)n3)ccc2c1 ; c1ccc2cc(-c3nc(-c4ccc5oc6ccccc6c5c4)nc(-c4cccc5ccc6ccccc6c45)n3)ccc2c1] 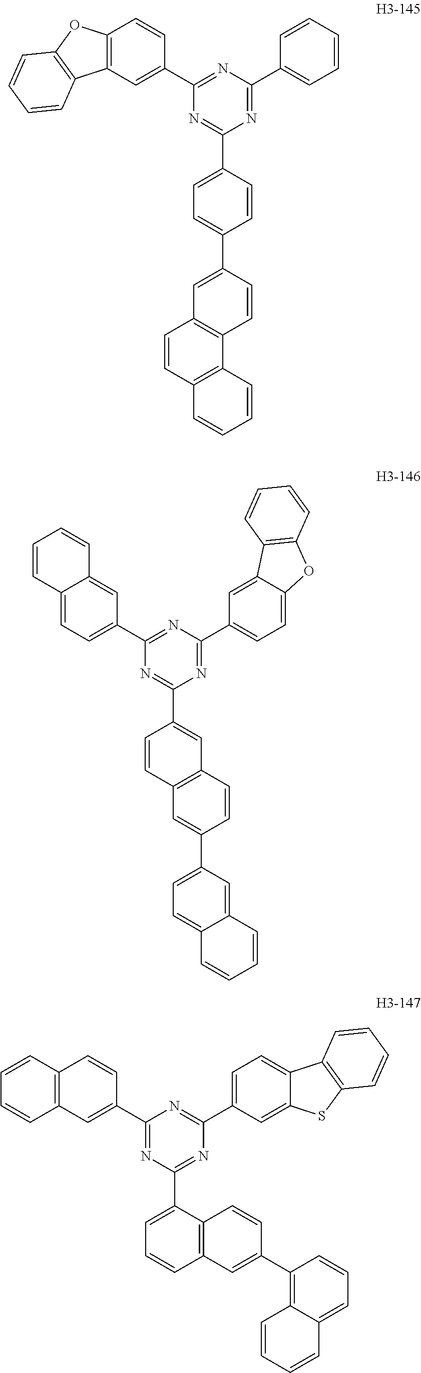 [SMILES: c1ccc(-c2nc(-c3ccc(-c4ccc5c(ccc6ccccc65)c4)cc3)nc(-c3ccc4oc5ccccc5c4c3)n2)cc1 ; c1ccc2cc(-c3ccc4cc(-c5nc(-c6ccc7ccccc7c6)nc(-c6ccc7oc8ccccc8c7c6)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1] 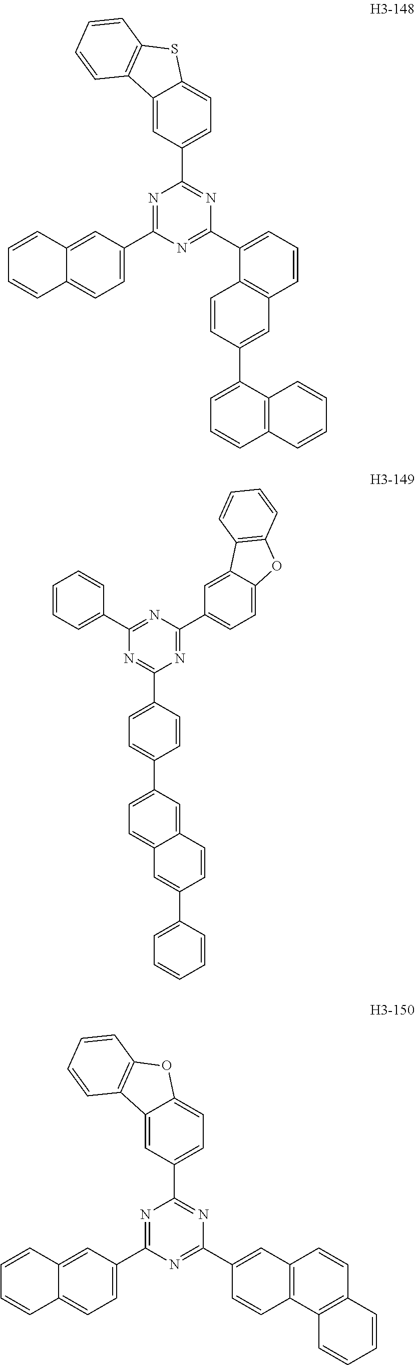 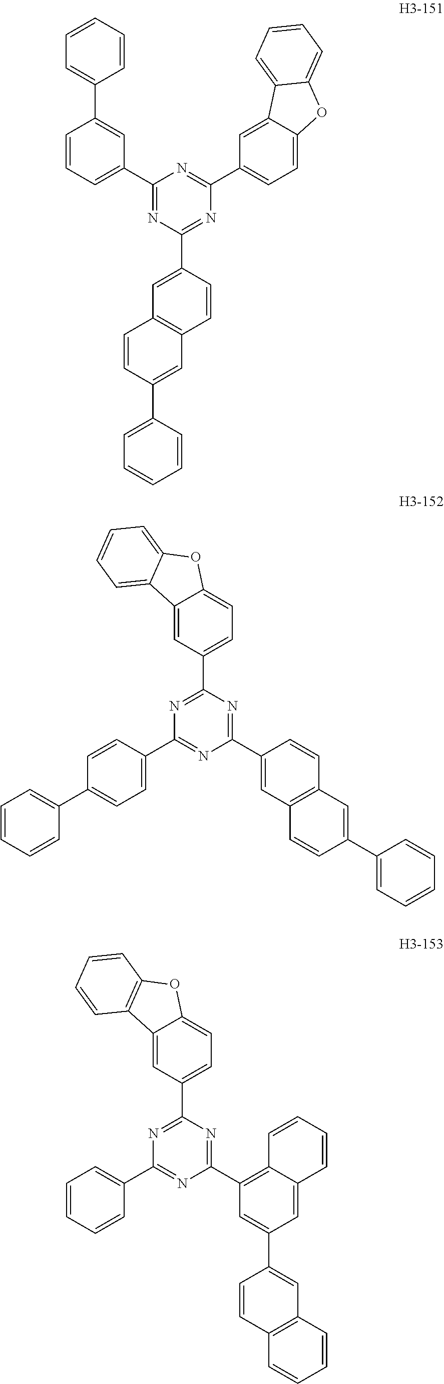 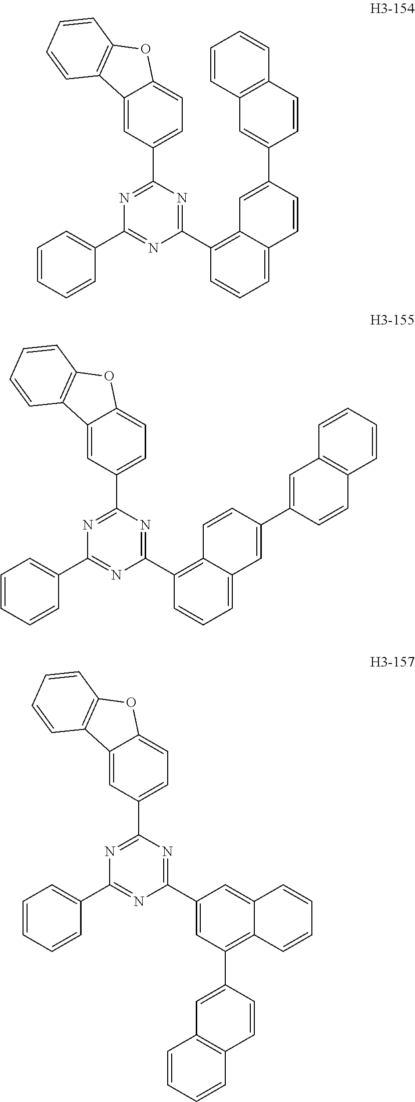 [SMILES: c1ccc(-c2nc(-c3ccc4oc5ccccc5c4c3)nc(-c3cccc4ccc(-c5ccc6ccccc6c5)cc34)n2)cc1 ; c1ccc(-c2nc(-c3ccc4oc5ccccc5c4c3)nc(-c3cccc4cc(-c5ccc6ccccc6c5)ccc34)n2)cc1 ; c1ccc(-c2nc(-c3cc(-c4ccc5ccccc5c4)c4ccccc4c3)nc(-c3ccc4oc5ccccc5c4c3)n2)cc1] 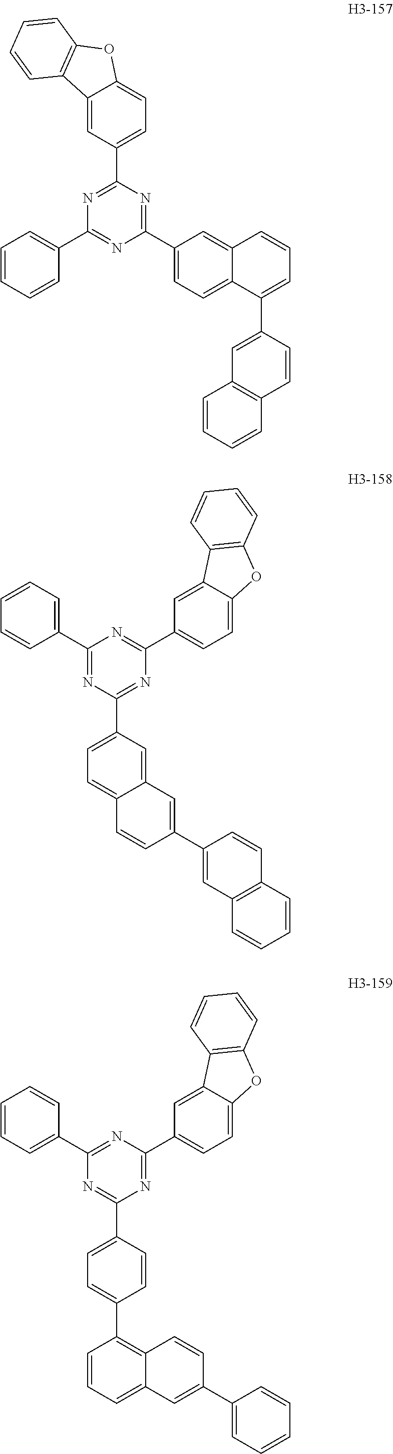 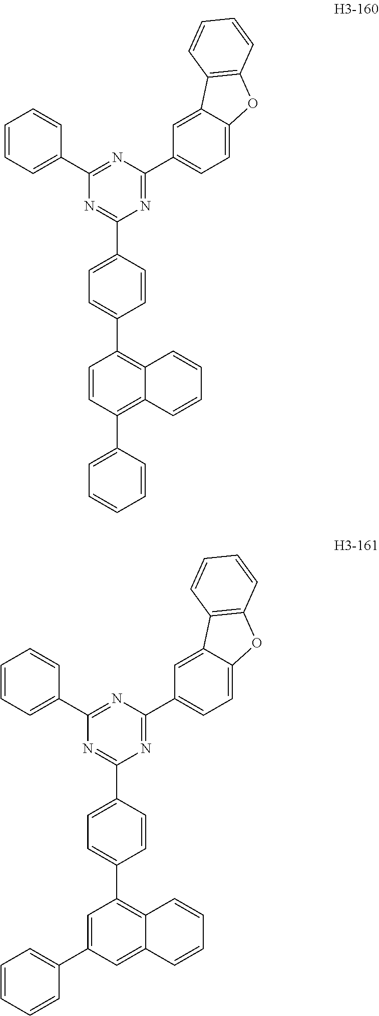 [SMILES: c1ccc(-c2nc(-c3ccc(-c4ccc(-c5ccccc5)c5ccccc45)cc3)nc(-c3ccc4oc5ccccc5c4c3)n2)cc1 ; c1ccc(-c2cc(-c3ccc(-c4nc(-c5ccccc5)nc(-c5ccc6oc7ccccc7c6c5)n4)cc3)c3ccccc3c2)cc1] 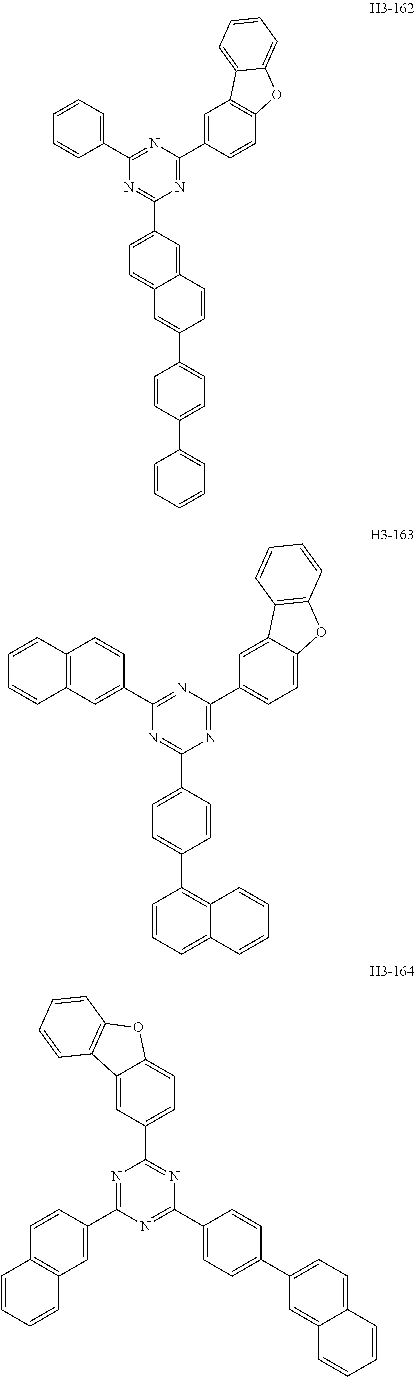 [SMILES: c1ccc(-c2ccc(-c3ccc4cc(-c5nc(-c6ccccc6)nc(-c6ccc7oc8ccccc8c7c6)n5)ccc4c3)cc2)cc1 ; c1ccc2cc(-c3nc(-c4ccc(-c5cccc6ccccc56)cc4)nc(-c4ccc5oc6ccccc6c5c4)n3)ccc2c1 ; c1ccc2cc(-c3ccc(-c4nc(-c5ccc6ccccc6c5)nc(-c5ccc6oc7ccccc7c6c5)n4)cc3)ccc2c1] 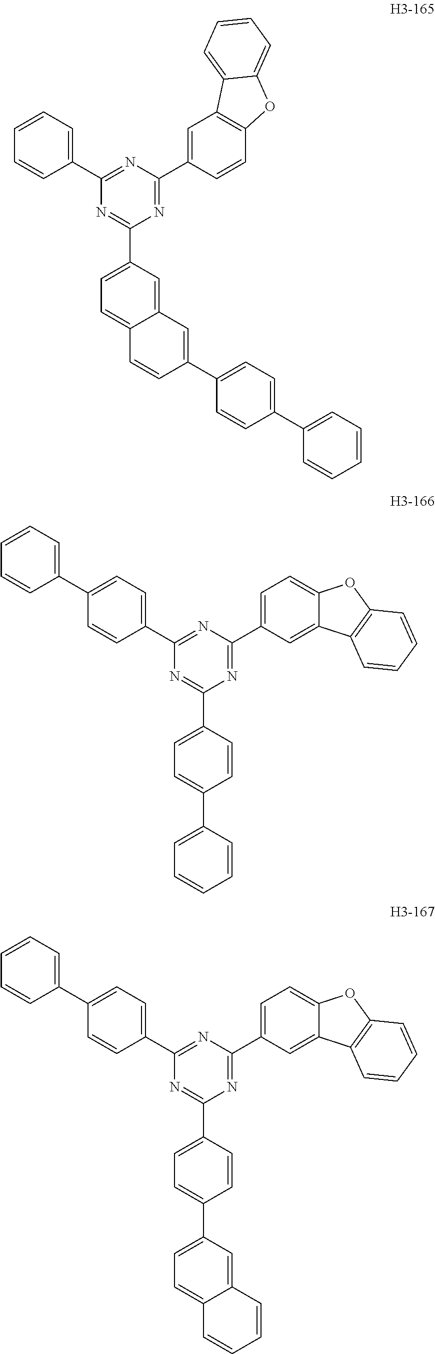 [SMILES: c1ccc(-c2ccc(-c3ccc4ccc(-c5nc(-c6ccccc6)nc(-c6ccc7oc8ccccc8c7c6)n5)cc4c3)cc2)cc1 ; c1ccc(-c2ccc(-c3nc(-c4ccc(-c5ccccc5)cc4)nc(-c4ccc5oc6ccccc6c5c4)n3)cc2)cc1 ; c1ccc(-c2ccc(-c3nc(-c4ccc(-c5ccc6ccccc6c5)cc4)nc(-c4ccc5oc6ccccc6c5c4)n3)cc2)cc1] 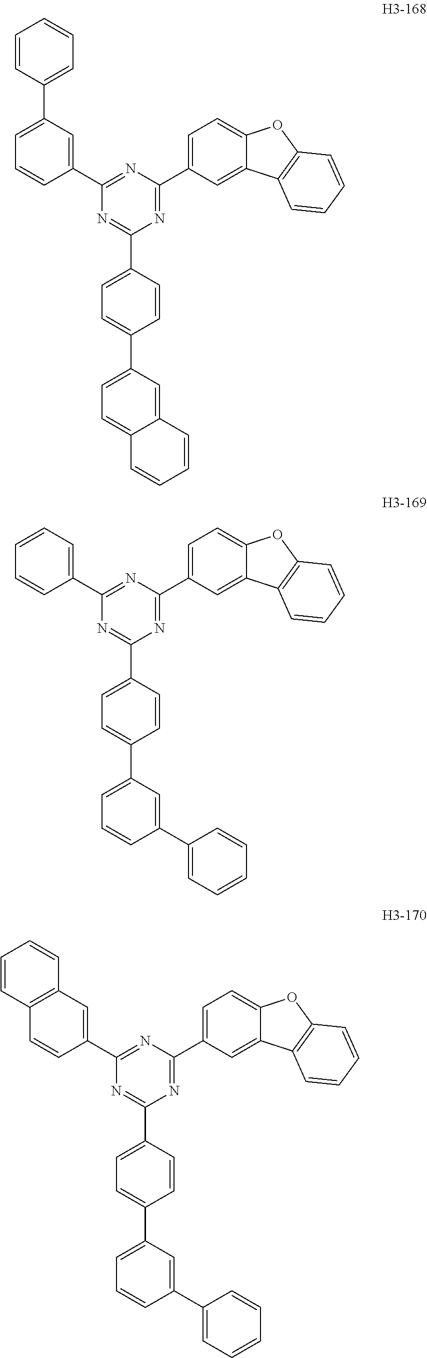 [SMILES: c1ccc(-c2cccc(-c3nc(-c4ccc(-c5ccc6ccccc6c5)cc4)nc(-c4ccc5oc6ccccc6c5c4)n3)c2)cc1 ; c1ccc(-c2cccc(-c3ccc(-c4nc(-c5ccccc5)nc(-c5ccc6oc7ccccc7c6c5)n4)cc3)c2)cc1 ; c1ccc(-c2cccc(-c3ccc(-c4nc(-c5ccc6ccccc6c5)nc(-c5ccc6oc7ccccc7c6c5)n4)cc3)c2)cc1] 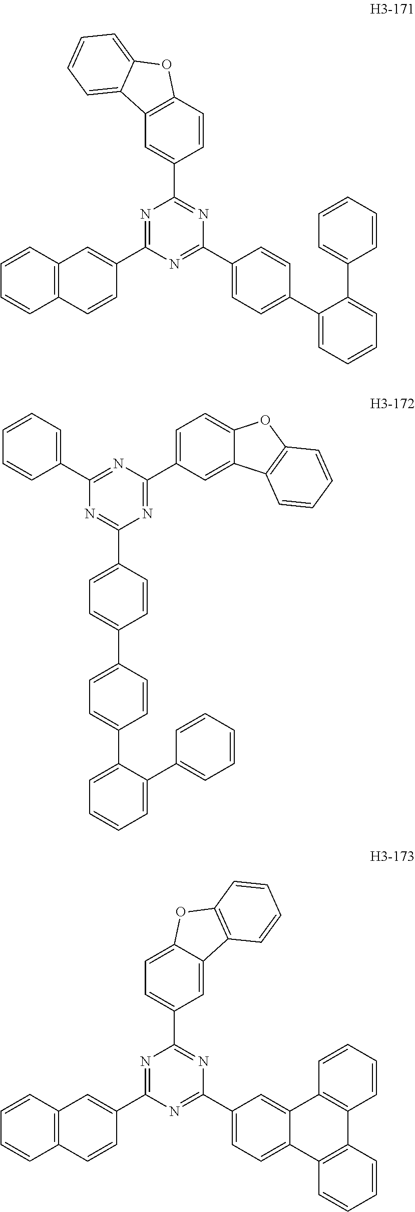 [SMILES: c1ccc(-c2ccccc2-c2ccc(-c3nc(-c4ccc5ccccc5c4)nc(-c4ccc5oc6ccccc6c5c4)n3)cc2)cc1 ; c1ccc(-c2nc(-c3ccc(-c4ccc(-c5ccccc5-c5ccccc5)cc4)cc3)nc(-c3ccc4oc5ccccc5c4c3)n2)cc1 ; c1ccc2cc(-c3nc(-c4ccc5oc6ccccc6c5c4)nc(-c4ccc5c6ccccc6c6ccccc6c5c4)n3)ccc2c1] 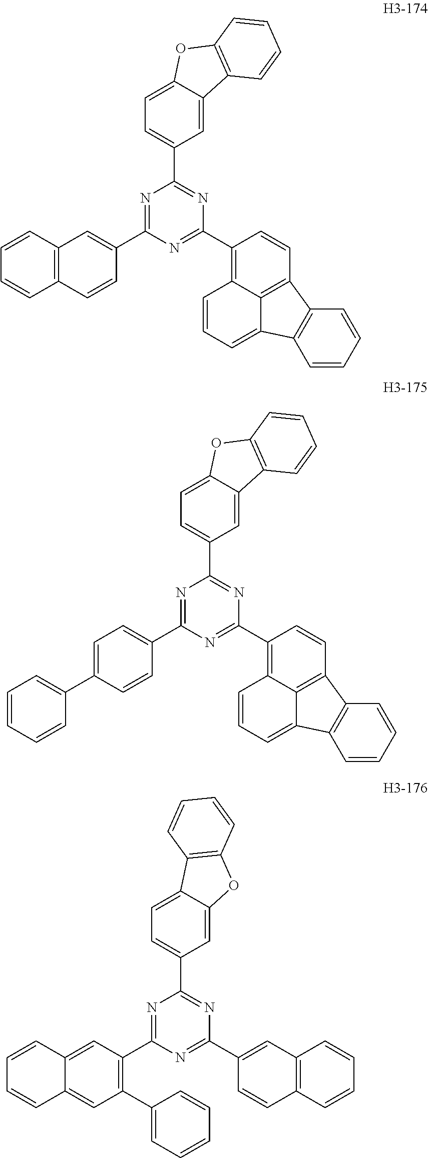 [SMILES: c1ccc2c(c1)-c1cccc3c(-c4nc(-c5ccc6ccccc6c5)nc(-c5ccc6oc7ccccc7c6c5)n4)ccc-2c13 ; c1ccc(-c2ccc(-c3nc(-c4ccc5oc6ccccc6c5c4)nc(-c4ccc5c6c(cccc46)-c4ccccc4-5)n3)cc2)cc1 ; c1ccc(-c2cc3ccccc3cc2-c2nc(-c3ccc4ccccc4c3)nc(-c3ccc4c(c3)oc3ccccc34)n2)cc1] 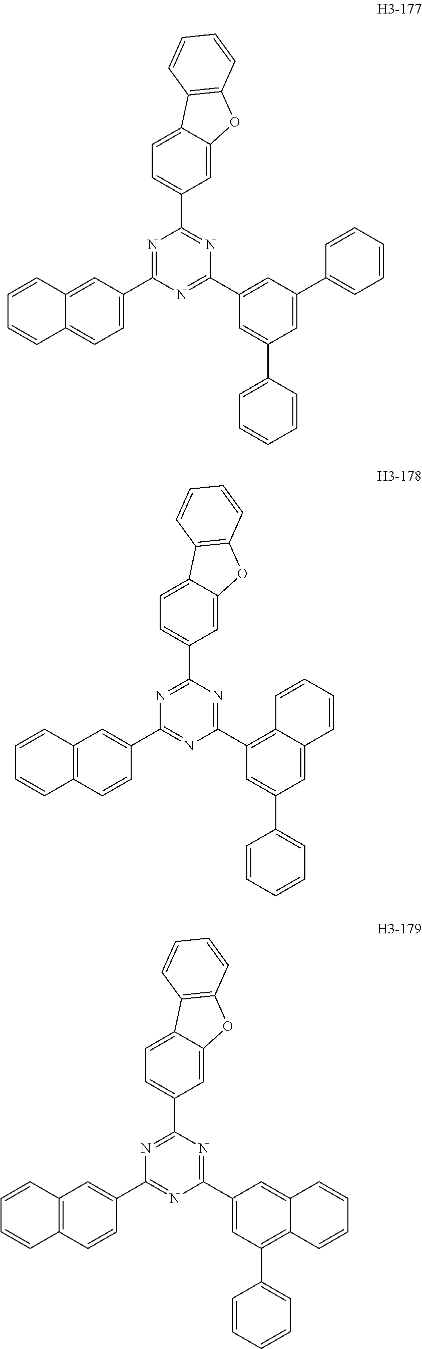 [SMILES: c1ccc(-c2cc(-c3ccccc3)cc(-c3nc(-c4ccc5ccccc5c4)nc(-c4ccc5c(c4)oc4ccccc45)n3)c2)cc1 ; c1ccc(-c2cc(-c3nc(-c4ccc5ccccc5c4)nc(-c4ccc5c(c4)oc4ccccc45)n3)c3ccccc3c2)cc1 ; c1ccc(-c2cc(-c3nc(-c4ccc5ccccc5c4)nc(-c4ccc5c(c4)oc4ccccc45)n3)cc3ccccc23)cc1] 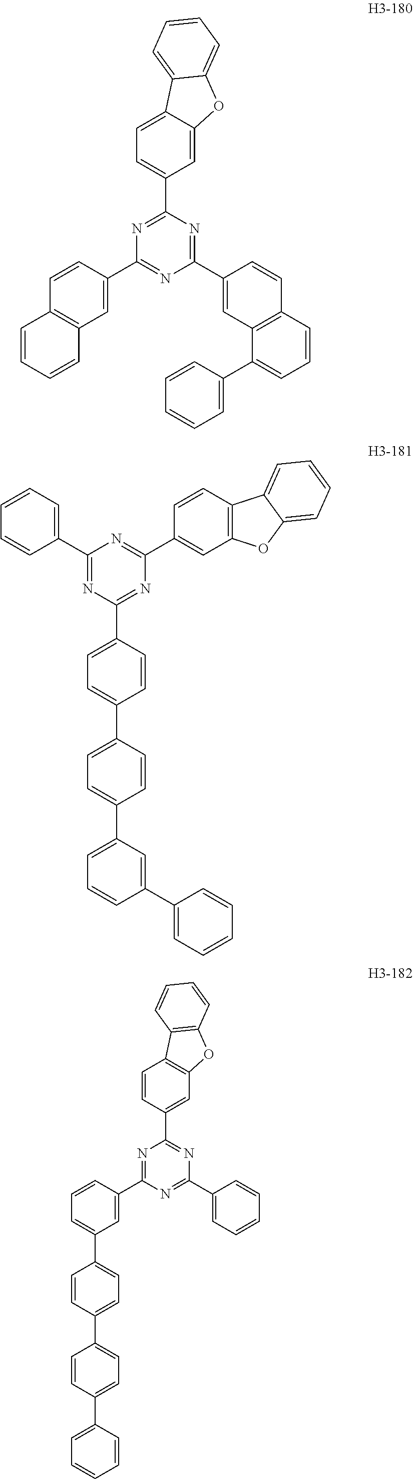 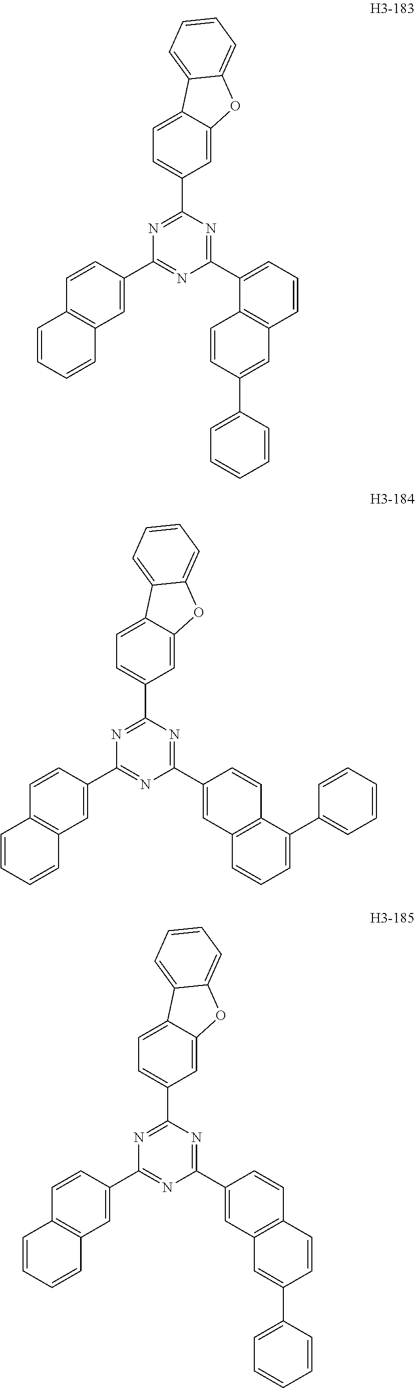 [SMILES: c1ccc(-c2ccc3c(-c4nc(-c5ccc6ccccc6c5)nc(-c5ccc6c(c5)oc5ccccc56)n4)cccc3c2)cc1 ; c1ccc(-c2cccc3cc(-c4nc(-c5ccc6ccccc6c5)nc(-c5ccc6c(c5)oc5ccccc56)n4)ccc23)cc1 ; c1ccc(-c2ccc3ccc(-c4nc(-c5ccc6ccccc6c5)nc(-c5ccc6c(c5)oc5ccccc56)n4)cc3c2)cc1] 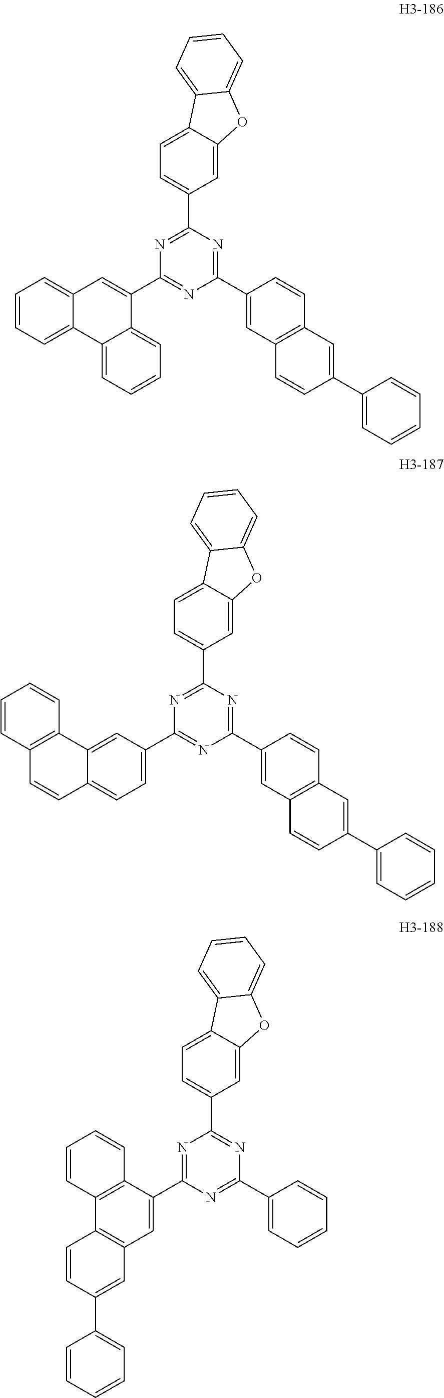 [SMILES: c1ccc(-c2ccc3cc(-c4nc(-c5ccc6c(c5)oc5ccccc56)nc(-c5cc6ccccc6c6ccccc56)n4)ccc3c2)cc1 ; c1ccc(-c2ccc3cc(-c4nc(-c5ccc6c(c5)oc5ccccc56)nc(-c5ccc6ccc7ccccc7c6c5)n4)ccc3c2)cc1 ; c1ccc(-c2ccc3c(c2)cc(-c2nc(-c4ccccc4)nc(-c4ccc5c(c4)oc4ccccc45)n2)c2ccccc23)cc1] 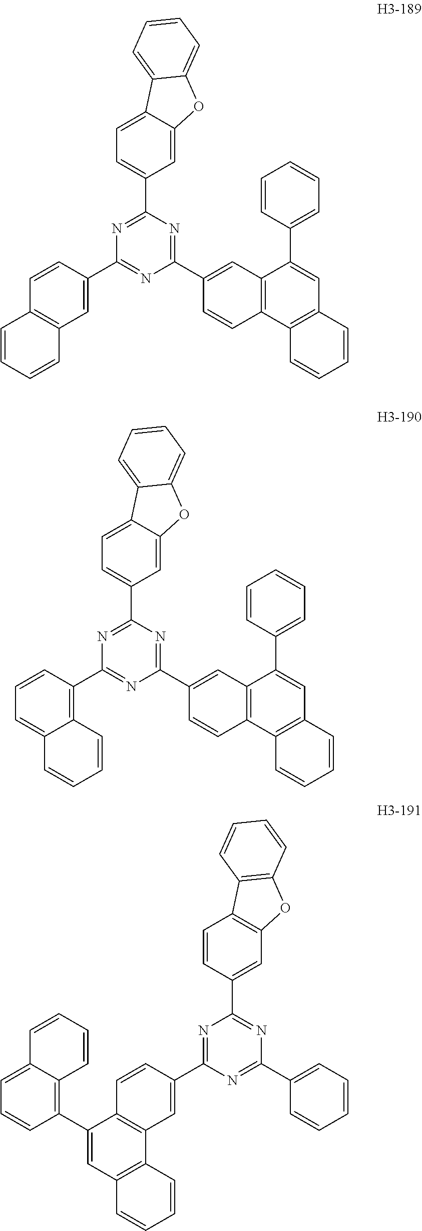 [SMILES: c1ccc(-c2cc3ccccc3c3ccc(-c4nc(-c5ccc6ccccc6c5)nc(-c5ccc6c(c5)oc5ccccc56)n4)cc23)cc1 ; c1ccc(-c2cc3ccccc3c3ccc(-c4nc(-c5ccc6c(c5)oc5ccccc56)nc(-c5cccc6ccccc56)n4)cc23)cc1 ; c1ccc(-c2nc(-c3ccc4c(c3)oc3ccccc34)nc(-c3ccc4c(-c5cccc6ccccc56)cc5ccccc5c4c3)n2)cc1] 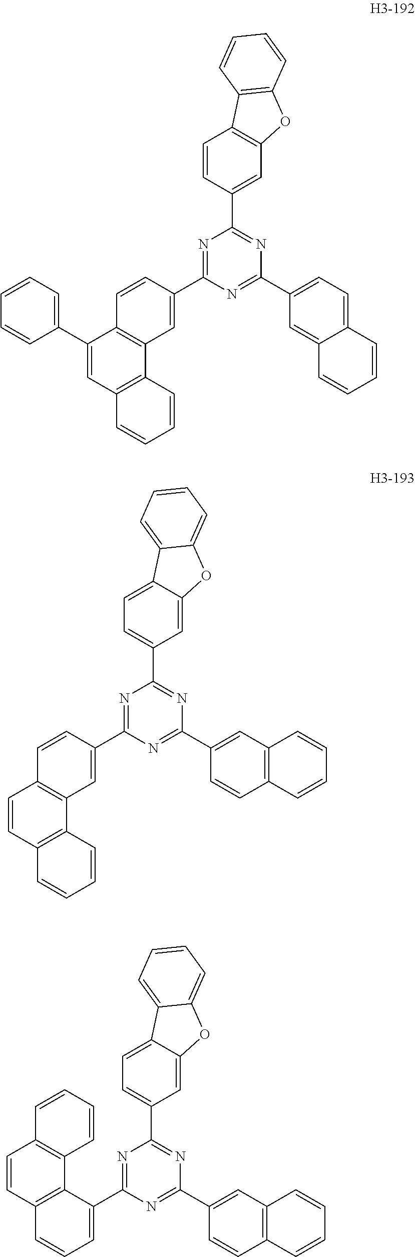 [SMILES: c1ccc(-c2cc3ccccc3c3cc(-c4nc(-c5ccc6ccccc6c5)nc(-c5ccc6c(c5)oc5ccccc56)n4)ccc23)cc1 ; c1ccc2cc(-c3nc(-c4ccc5c(c4)oc4ccccc45)nc(-c4ccc5ccc6ccccc6c5c4)n3)ccc2c1 ; c1ccc2cc(-c3nc(-c4ccc5c(c4)oc4ccccc45)nc(-c4cccc5ccc6ccccc6c45)n3)ccc2c1] 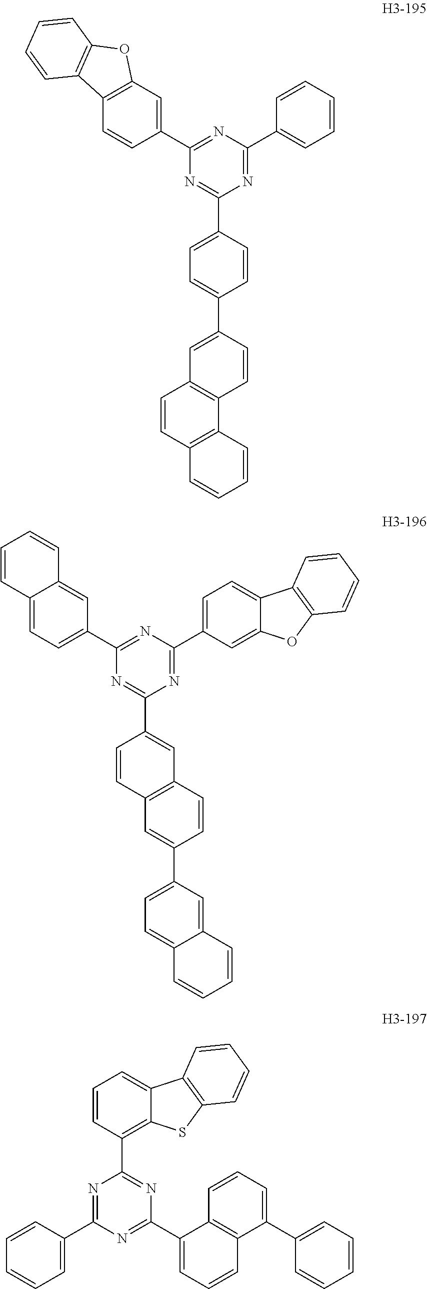 [SMILES: c1ccc(-c2nc(-c3ccc(-c4ccc5c(ccc6ccccc65)c4)cc3)nc(-c3ccc4c(c3)oc3ccccc34)n2)cc1 ; c1ccc2cc(-c3ccc4cc(-c5nc(-c6ccc7ccccc7c6)nc(-c6ccc7c(c6)oc6ccccc67)n5)ccc4c3)ccc2c1 ; c1ccc(-c2nc(-c3cccc4c(-c5ccccc5)cccc34)nc(-c3cccc4c3sc3ccccc34)n2)cc1] 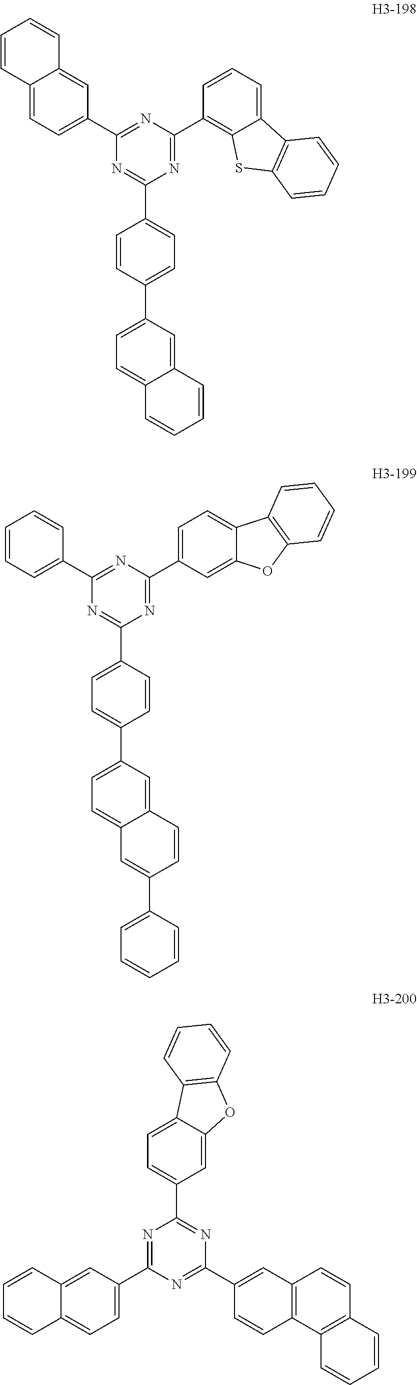 [SMILES: c1ccc2cc(-c3ccc(-c4nc(-c5ccc6ccccc6c5)nc(-c5cccc6c5sc5ccccc56)n4)cc3)ccc2c1 ; c1ccc(-c2ccc3cc(-c4ccc(-c5nc(-c6ccccc6)nc(-c6ccc7c(c6)oc6ccccc67)n5)cc4)ccc3c2)cc1 ; c1ccc2cc(-c3nc(-c4ccc5c(ccc6ccccc65)c4)nc(-c4ccc5c(c4)oc4ccccc45)n3)ccc2c1] 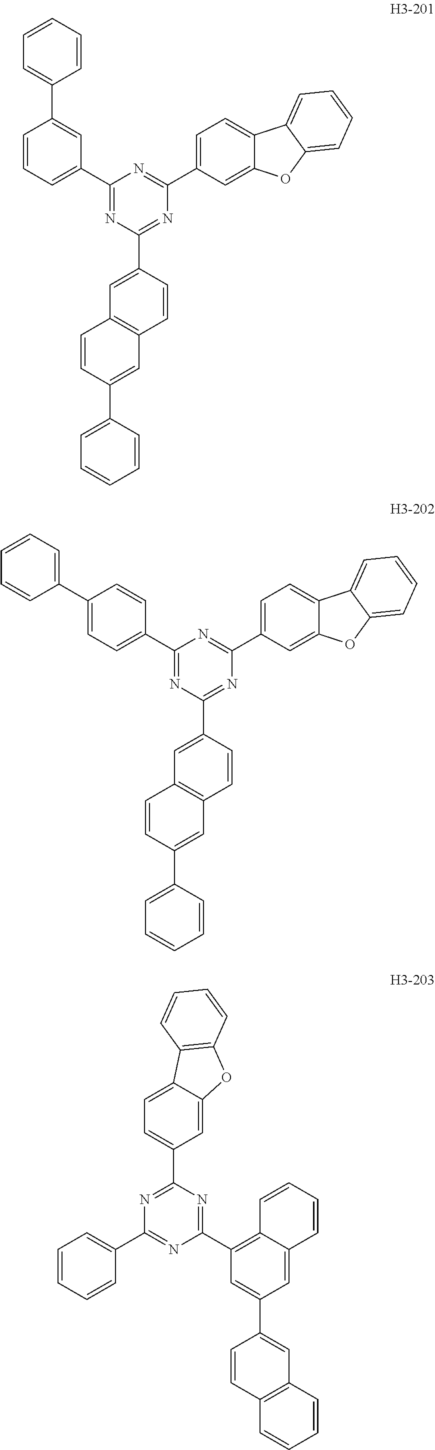 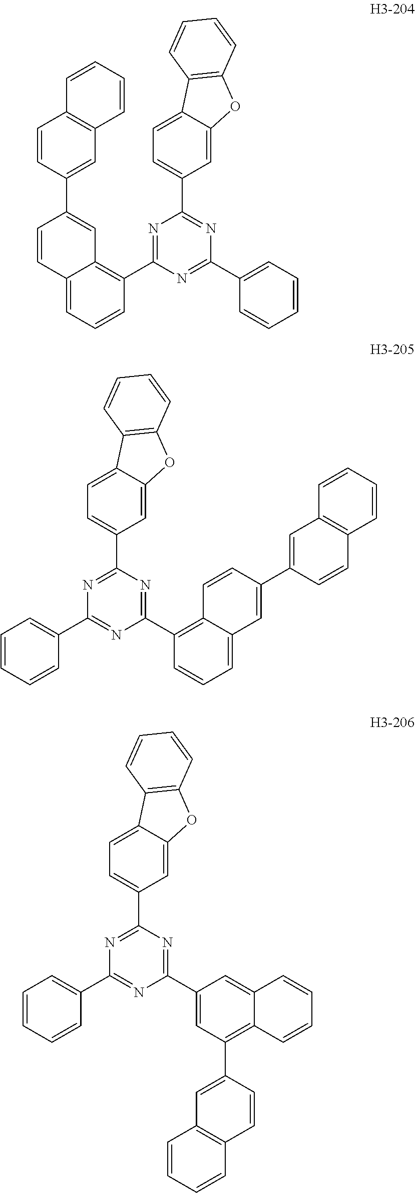 [SMILES: c1ccc(-c2nc(-c3ccc4c(c3)oc3ccccc34)nc(-c3cccc4ccc(-c5ccc6ccccc6c5)cc34)n2)cc1 ; c1ccc(-c2nc(-c3ccc4c(c3)oc3ccccc34)nc(-c3cccc4cc(-c5ccc6ccccc6c5)ccc34)n2)cc1 ; c1ccc(-c2nc(-c3cc(-c4ccc5ccccc5c4)c4ccccc4c3)nc(-c3ccc4c(c3)oc3ccccc34)n2)cc1] 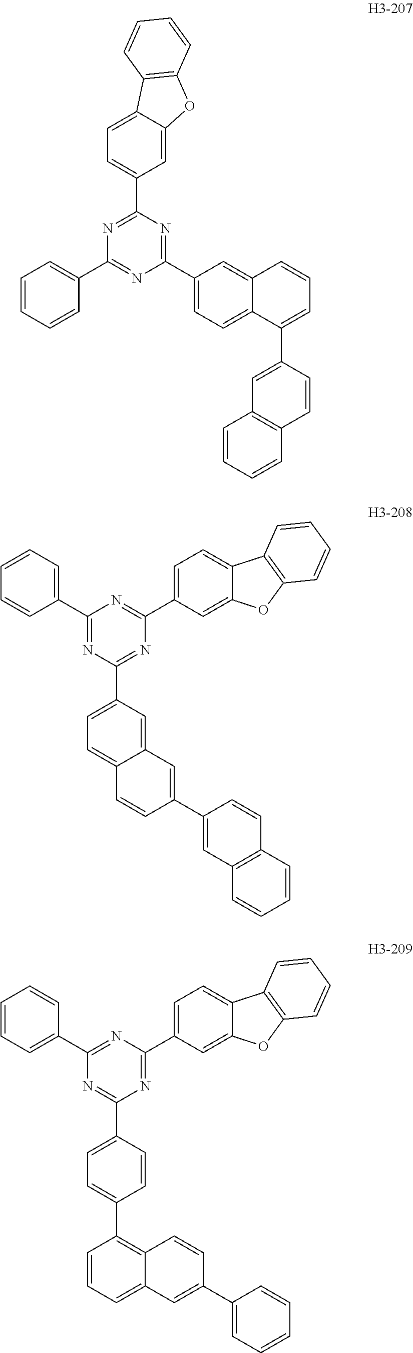 [SMILES: c1ccc(-c2nc(-c3ccc4c(-c5ccc6ccccc6c5)cccc4c3)nc(-c3ccc4c(c3)oc3ccccc34)n2)cc1 ; c1ccc(-c2nc(-c3ccc4ccc(-c5ccc6ccccc6c5)cc4c3)nc(-c3ccc4c(c3)oc3ccccc34)n2)cc1 ; c1ccc(-c2ccc3c(-c4ccc(-c5nc(-c6ccccc6)nc(-c6ccc7c(c6)oc6ccccc67)n5)cc4)cccc3c2)cc1] 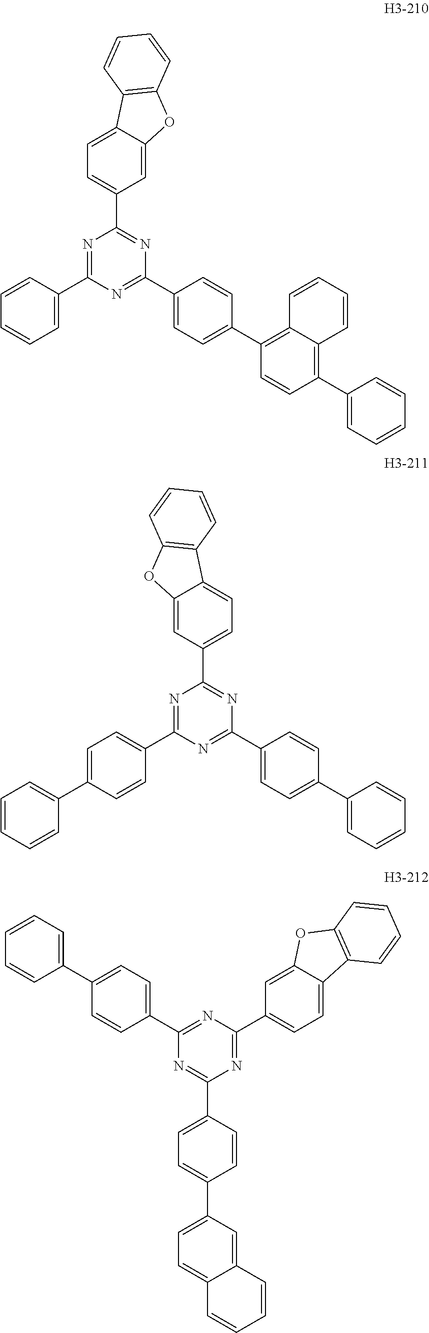 [SMILES: c1ccc(-c2nc(-c3ccc(-c4ccc(-c5ccccc5)c5ccccc45)cc3)nc(-c3ccc4c(c3)oc3ccccc34)n2)cc1 ; c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4ccc5c(c4)oc4ccccc45)n3)cc2)cc1] 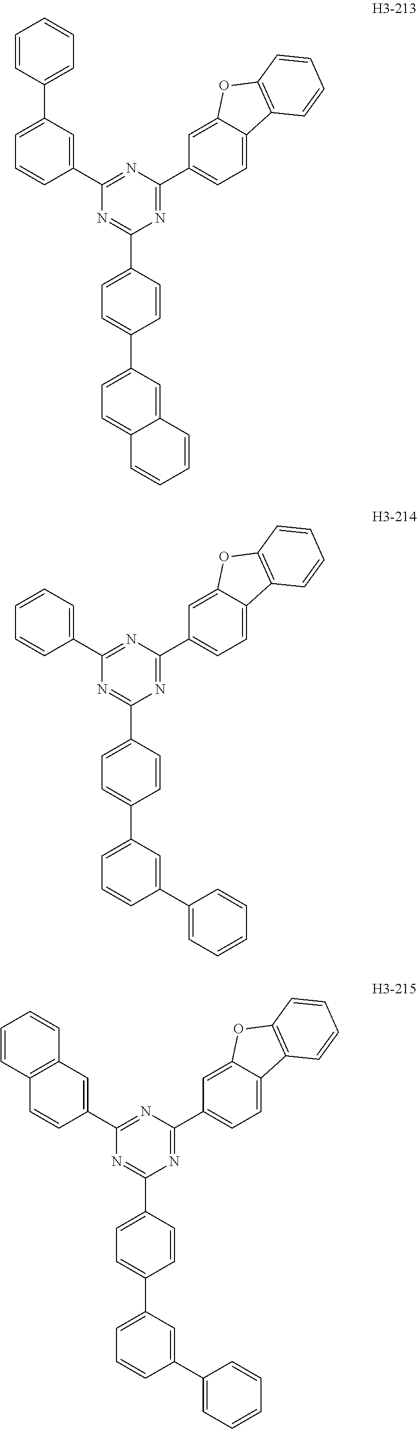 [SMILES: c1ccc(-c2cccc(-c3nc(-c4ccc(-c5ccc6ccccc6c5)cc4)nc(-c4ccc5c(c4)oc4ccccc45)n3)c2)cc1 ; c1ccc(-c2cccc(-c3ccc(-c4nc(-c5ccccc5)nc(-c5ccc6c(c5)oc5ccccc56)n4)cc3)c2)cc1 ; c1ccc(-c2cccc(-c3ccc(-c4nc(-c5ccc6ccccc6c5)nc(-c5ccc6c(c5)oc5ccccc56)n4)cc3)c2)cc1] 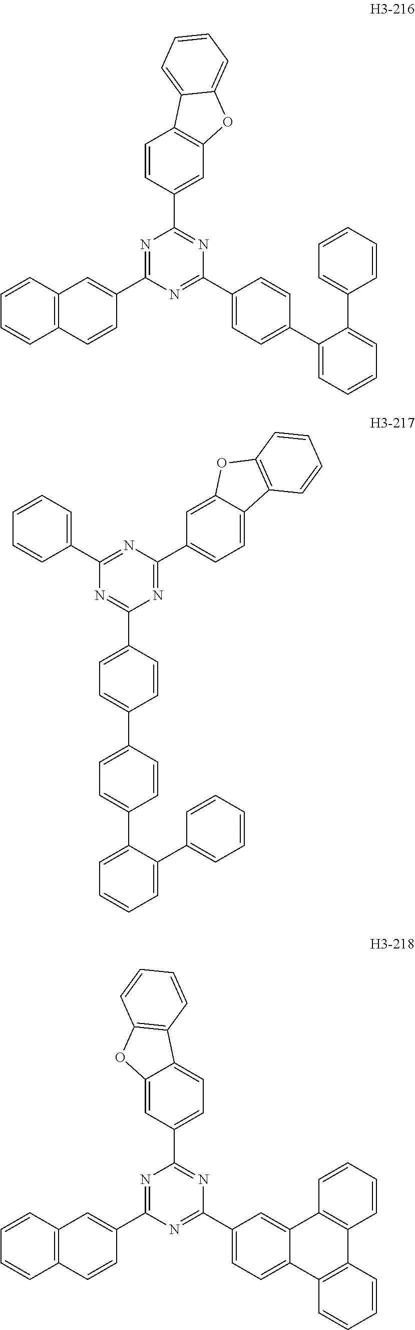 [SMILES: c1ccc(-c2ccccc2-c2ccc(-c3nc(-c4ccc5ccccc5c4)nc(-c4ccc5c(c4)oc4ccccc45)n3)cc2)cc1 ; c1ccc(-c2nc(-c3ccc(-c4ccc(-c5ccccc5-c5ccccc5)cc4)cc3)nc(-c3ccc4c(c3)oc3ccccc34)n2)cc1 ; c1ccc2cc(-c3nc(-c4ccc5c(c4)oc4ccccc45)nc(-c4ccc5c6ccccc6c6ccccc6c5c4)n3)ccc2c1] 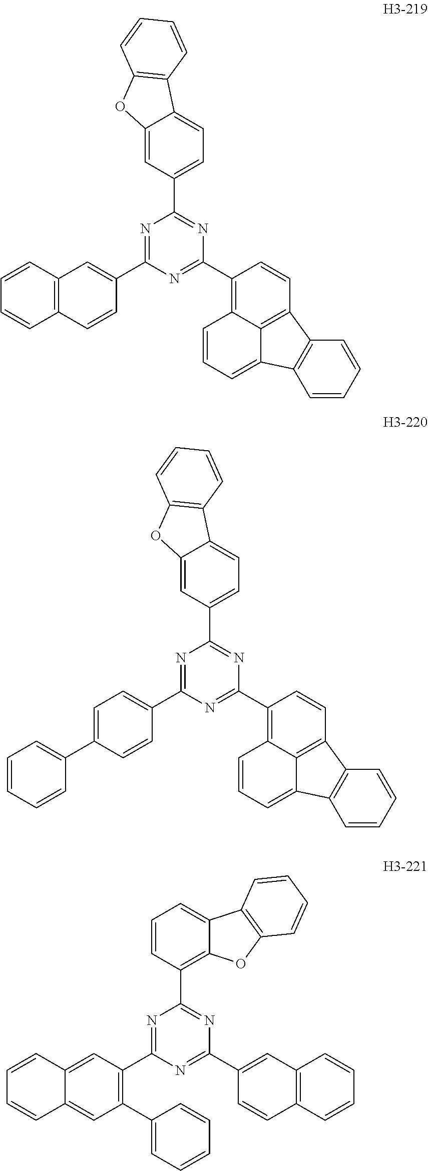 [SMILES: c1ccc2c(c1)-c1cccc3c(-c4nc(-c5ccc6ccccc6c5)nc(-c5ccc6c(c5)oc5ccccc56)n4)ccc-2c13 ; c1ccc(-c2ccc(-c3nc(-c4ccc5c(c4)oc4ccccc45)nc(-c4ccc5c6c(cccc46)-c4ccccc4-5)n3)cc2)cc1 ; c1ccc(-c2cc3ccccc3cc2-c2nc(-c3ccc4ccccc4c3)nc(-c3cccc4c3oc3ccccc34)n2)cc1] 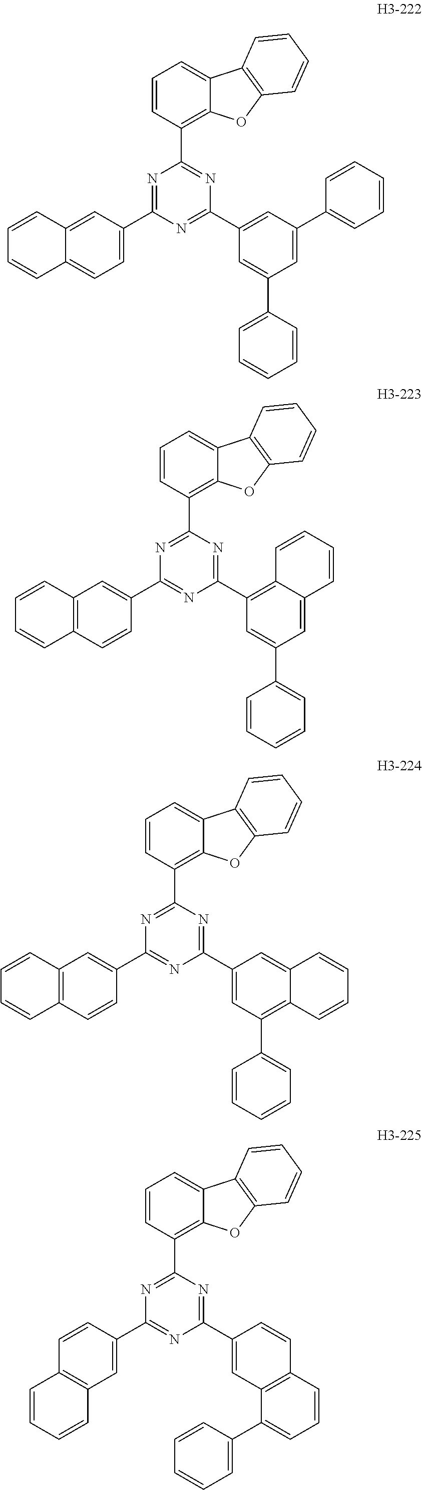 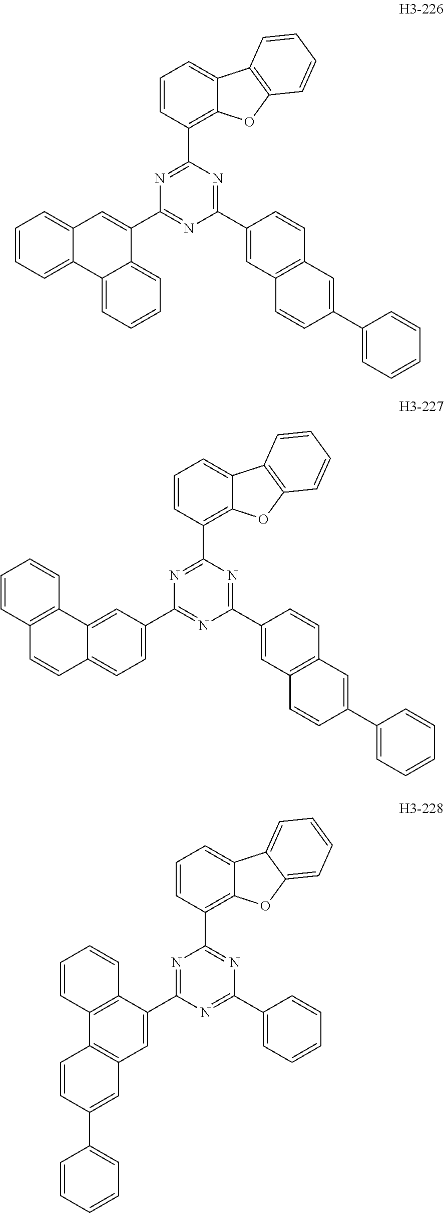 [SMILES: c1ccc(-c2ccc3cc(-c4nc(-c5cc6ccccc6c6ccccc56)nc(-c5cccc6c5oc5ccccc56)n4)ccc3c2)cc1 ; c1ccc(-c2ccc3cc(-c4nc(-c5ccc6ccc7ccccc7c6c5)nc(-c5cccc6c5oc5ccccc56)n4)ccc3c2)cc1 ; c1ccc(-c2ccc3c(c2)cc(-c2nc(-c4ccccc4)nc(-c4cccc5c4oc4ccccc45)n2)c2ccccc23)cc1] 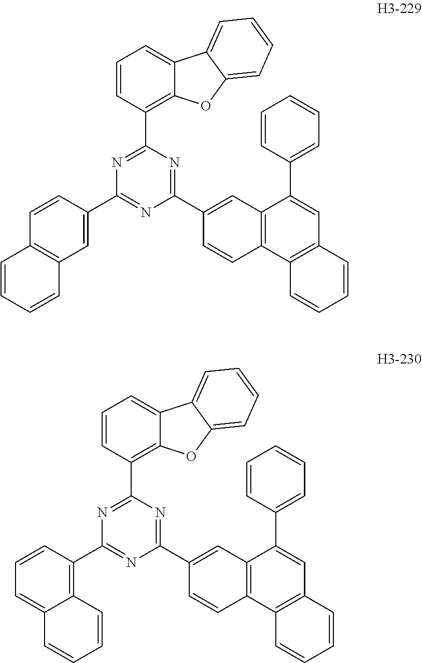 [SMILES: c1ccc(-c2cc3ccccc3c3ccc(-c4nc(-c5ccc6ccccc6c5)nc(-c5cccc6c5oc5ccccc56)n4)cc23)cc1 ; c1ccc(-c2cc3ccccc3c3ccc(-c4nc(-c5cccc6ccccc56)nc(-c5cccc6c5oc5ccccc56)n4)cc23)cc1] 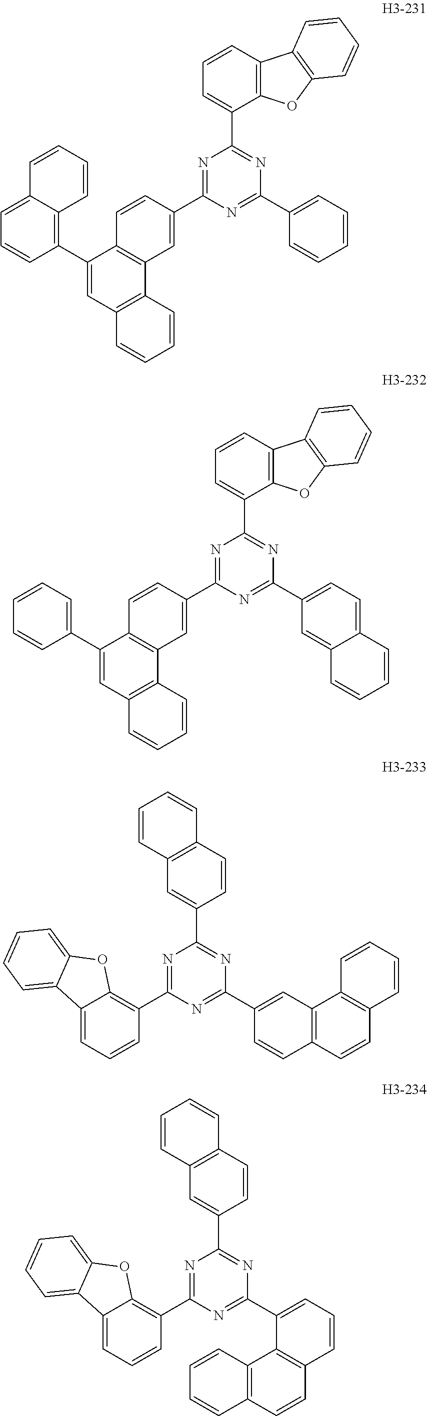 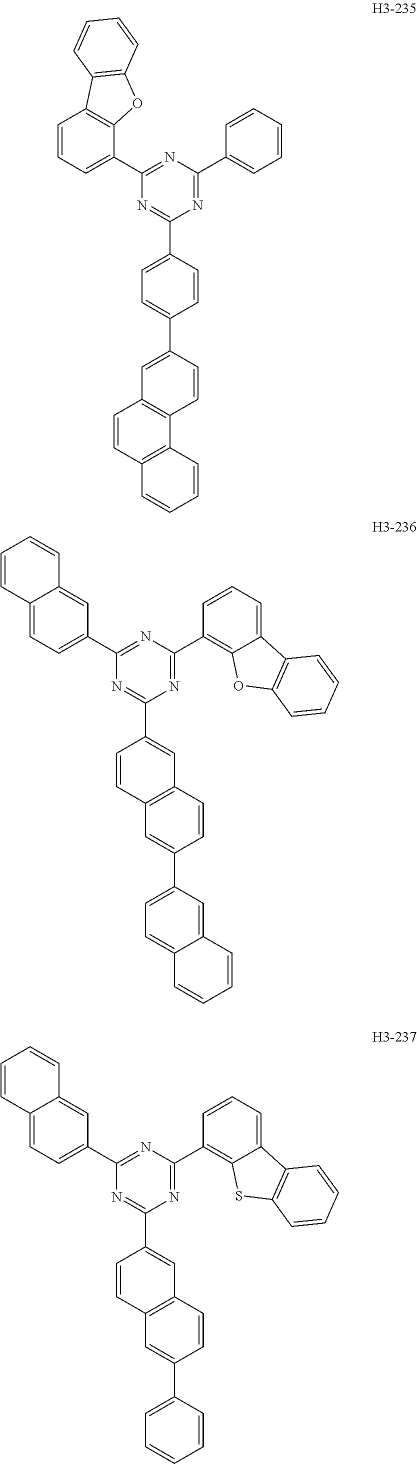 [SMILES: c1ccc(-c2nc(-c3ccc(-c4ccc5c(ccc6ccccc65)c4)cc3)nc(-c3cccc4c3oc3ccccc34)n2)cc1 ; c1ccc2cc(-c3ccc4cc(-c5nc(-c6ccc7ccccc7c6)nc(-c6cccc7c6oc6ccccc67)n5)ccc4c3)ccc2c1 ; c1ccc(-c2ccc3cc(-c4nc(-c5ccc6ccccc6c5)nc(-c5cccc6c5sc5ccccc56)n4)ccc3c2)cc1] 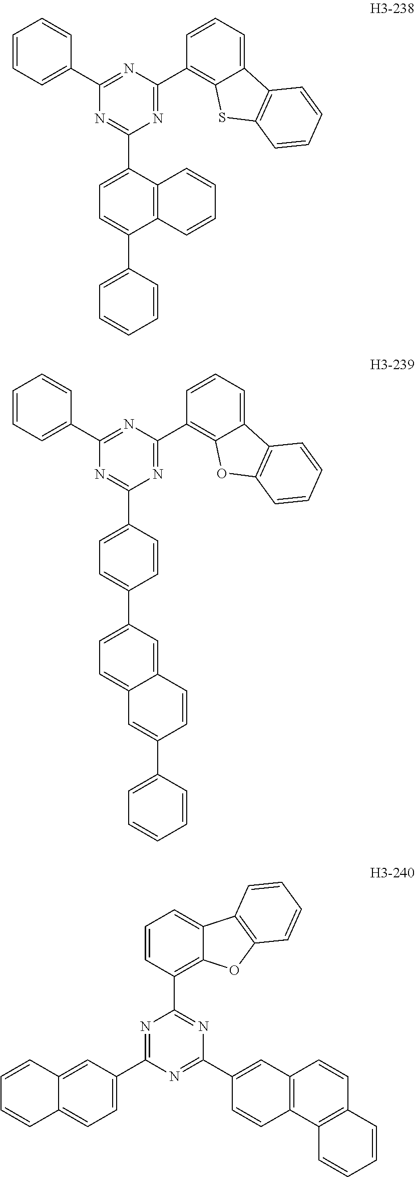 [SMILES: c1ccc(-c2nc(-c3ccc(-c4ccccc4)c4ccccc34)nc(-c3cccc4c3sc3ccccc34)n2)cc1 ; c1ccc(-c2ccc3cc(-c4ccc(-c5nc(-c6ccccc6)nc(-c6cccc7c6oc6ccccc67)n5)cc4)ccc3c2)cc1 ; c1ccc2cc(-c3nc(-c4ccc5c(ccc6ccccc65)c4)nc(-c4cccc5c4oc4ccccc45)n3)ccc2c1] 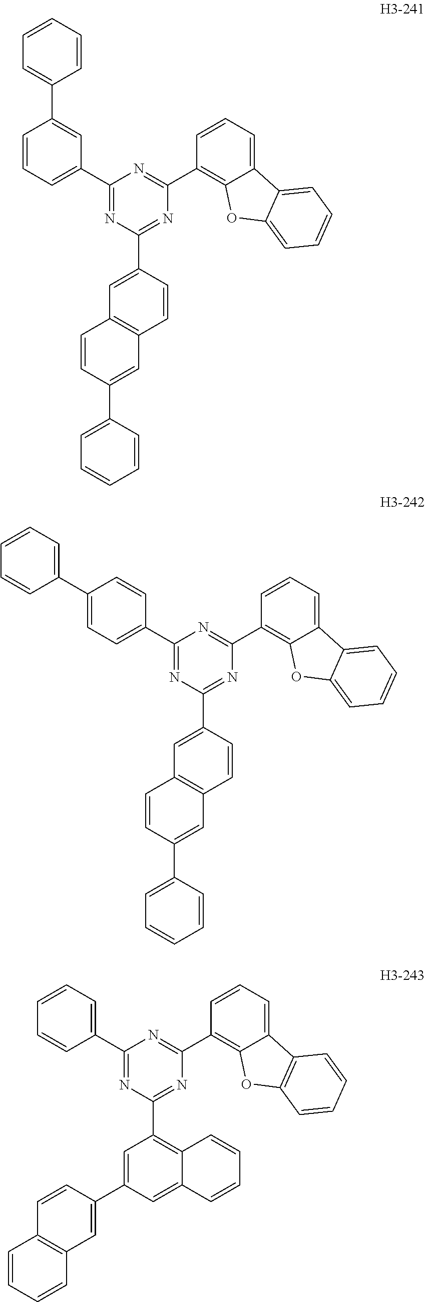 [SMILES: c1ccc(-c2cccc(-c3nc(-c4ccc5cc(-c6ccccc6)ccc5c4)nc(-c4cccc5c4oc4ccccc45)n3)c2)cc1 ; c1ccc(-c2ccc(-c3nc(-c4ccc5cc(-c6ccccc6)ccc5c4)nc(-c4cccc5c4oc4ccccc45)n3)cc2)cc1 ; c1ccc(-c2nc(-c3cc(-c4ccc5ccccc5c4)cc4ccccc34)nc(-c3cccc4c3oc3ccccc34)n2)cc1] 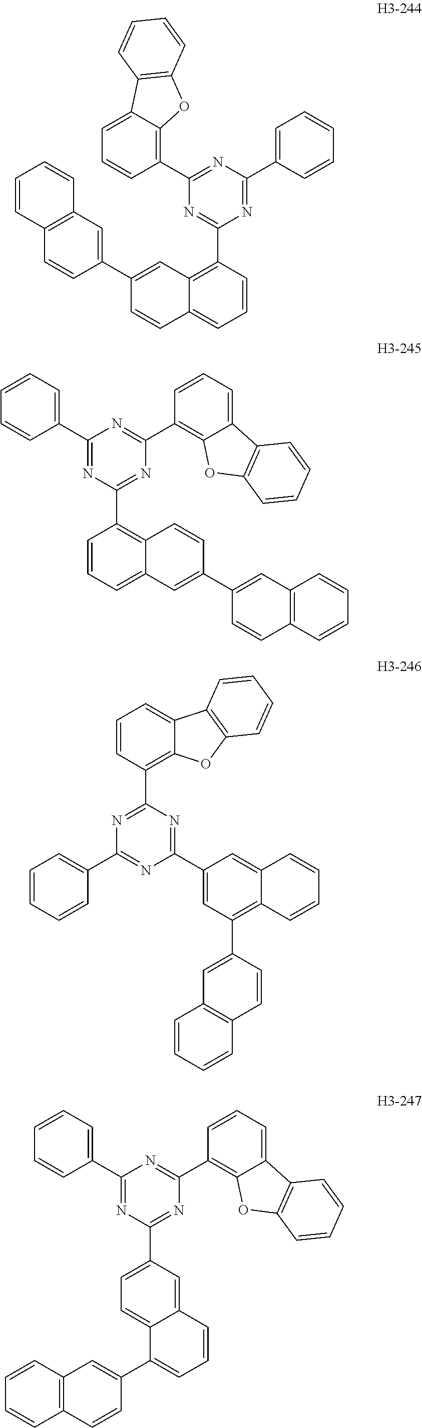 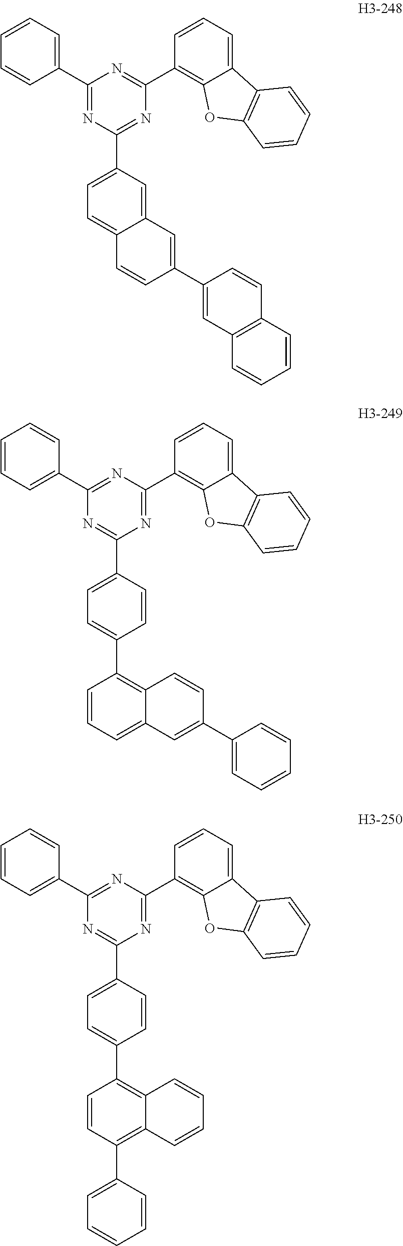 [SMILES: c1ccc(-c2nc(-c3ccc4ccc(-c5ccc6ccccc6c5)cc4c3)nc(-c3cccc4c3oc3ccccc34)n2)cc1 ; c1ccc(-c2ccc3c(-c4ccc(-c5nc(-c6ccccc6)nc(-c6cccc7c6oc6ccccc67)n5)cc4)cccc3c2)cc1 ; c1ccc(-c2nc(-c3ccc(-c4ccc(-c5ccccc5)c5ccccc45)cc3)nc(-c3cccc4c3oc3ccccc34)n2)cc1] 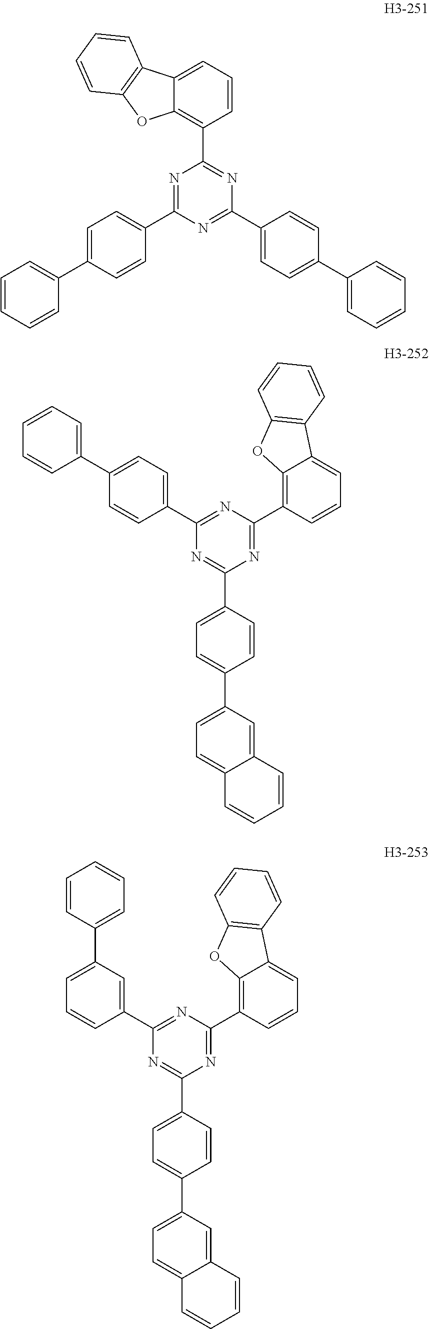 [SMILES: c1ccc(-c2ccc(-c3nc(-c4ccc(-c5ccccc5)cc4)nc(-c4cccc5c4oc4ccccc45)n3)cc2)cc1 ; c1ccc(-c2ccc(-c3nc(-c4ccc(-c5ccc6ccccc6c5)cc4)nc(-c4cccc5c4oc4ccccc45)n3)cc2)cc1 ; c1ccc(-c2cccc(-c3nc(-c4ccc(-c5ccc6ccccc6c5)cc4)nc(-c4cccc5c4oc4ccccc45)n3)c2)cc1] 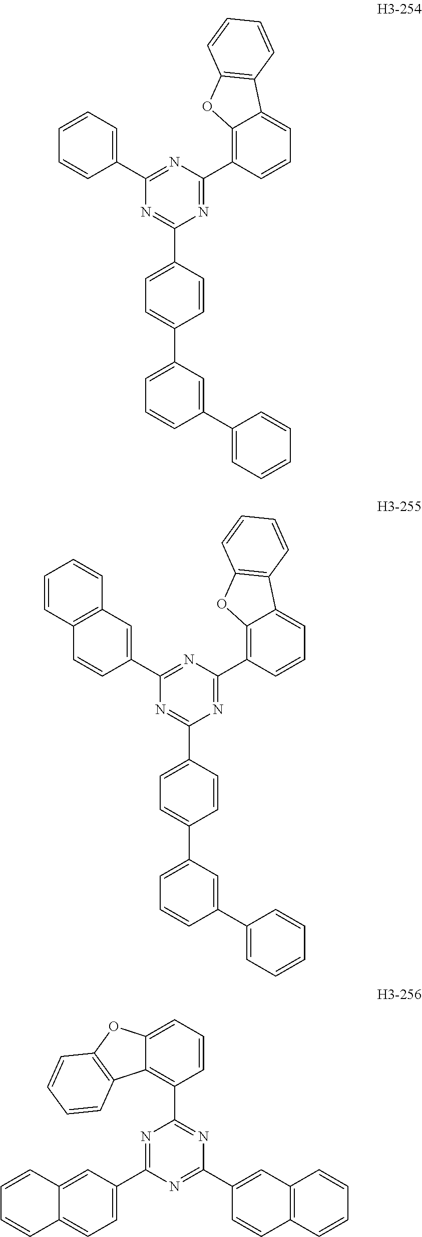 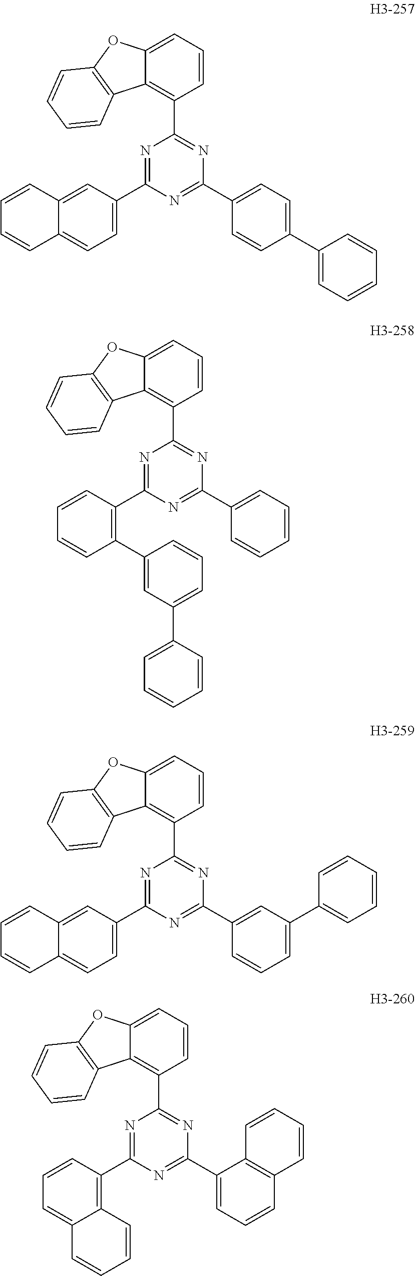 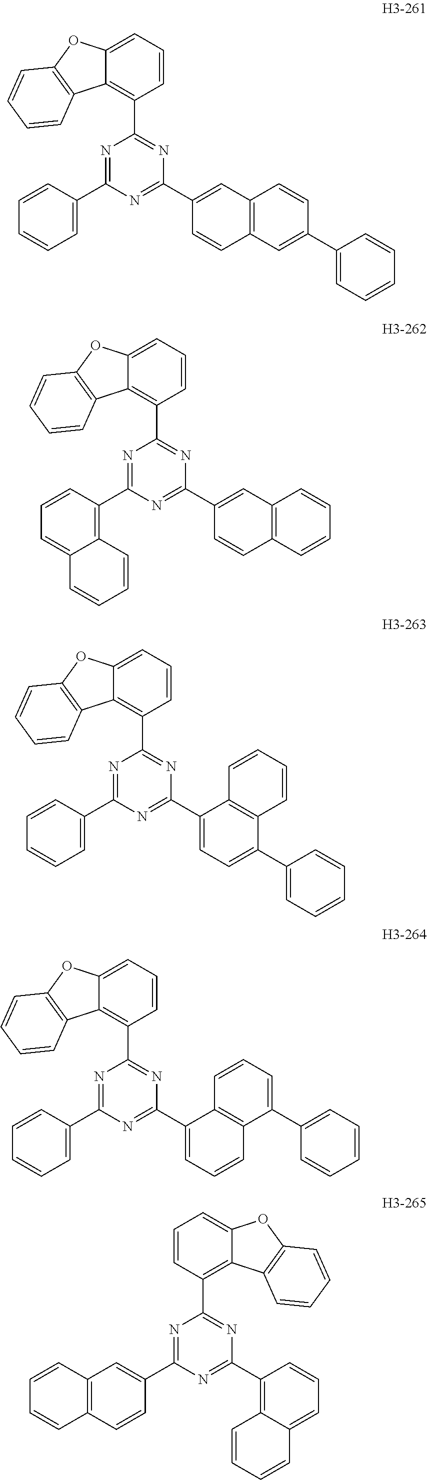 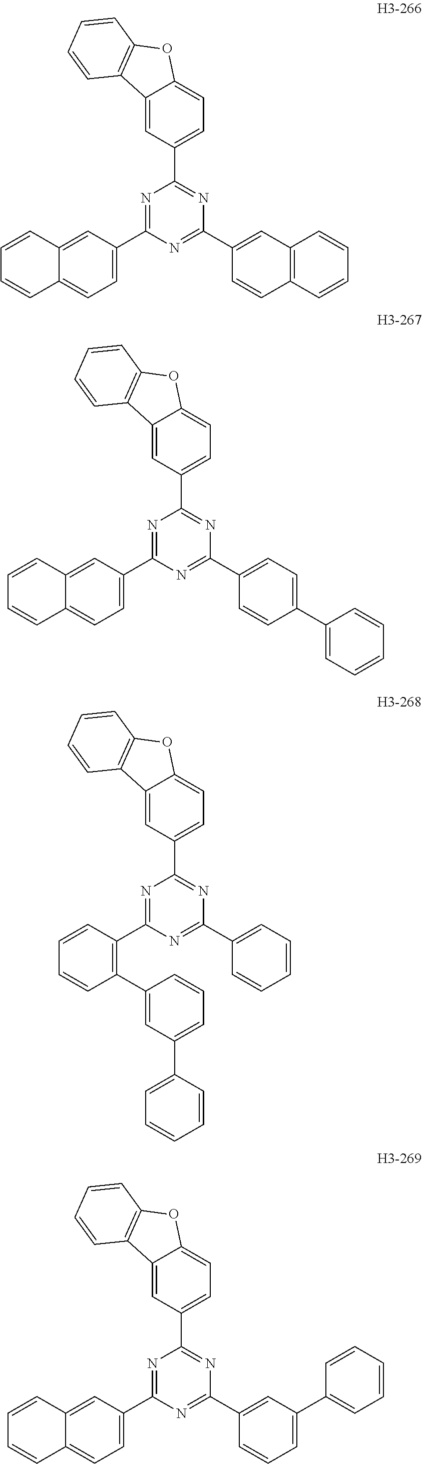 [SMILES: c1ccc2cc(-c3nc(-c4ccc5ccccc5c4)nc(-c4ccc5oc6ccccc6c5c4)n3)ccc2c1 ; c1ccc(-c2ccc(-c3nc(-c4ccc5ccccc5c4)nc(-c4ccc5oc6ccccc6c5c4)n3)cc2)cc1 ; c1ccc(-c2cccc(-c3ccccc3-c3nc(-c4ccccc4)nc(-c4ccc5oc6ccccc6c5c4)n3)c2)cc1 ; c1ccc(-c2cccc(-c3nc(-c4ccc5ccccc5c4)nc(-c4ccc5oc6ccccc6c5c4)n3)c2)cc1] 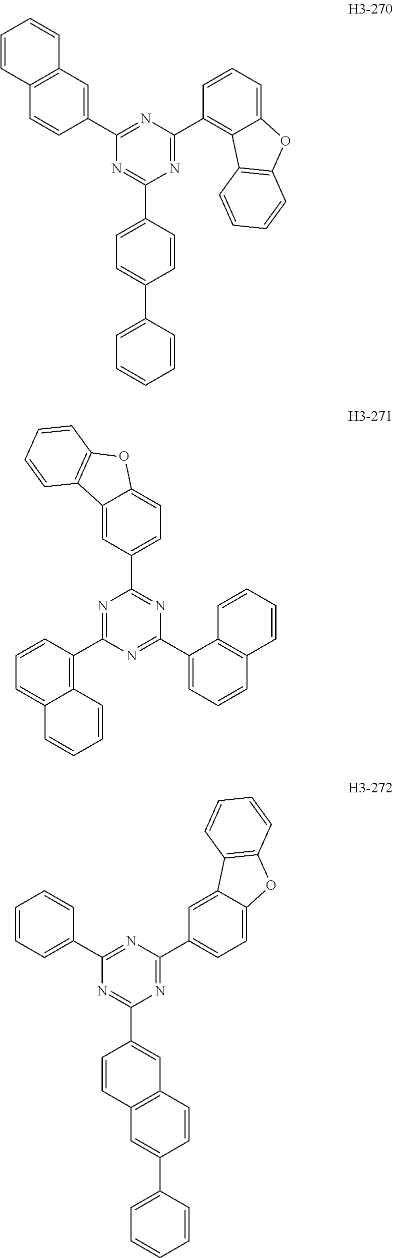 [SMILES: c1ccc(-c2ccc(-c3nc(-c4ccc5ccccc5c4)nc(-c4cccc5oc6ccccc6c45)n3)cc2)cc1 ; c1ccc2c(-c3nc(-c4ccc5oc6ccccc6c5c4)nc(-c4cccc5ccccc45)n3)cccc2c1 ; c1ccc(-c2ccc3cc(-c4nc(-c5ccccc5)nc(-c5ccc6oc7ccccc7c6c5)n4)ccc3c2)cc1] 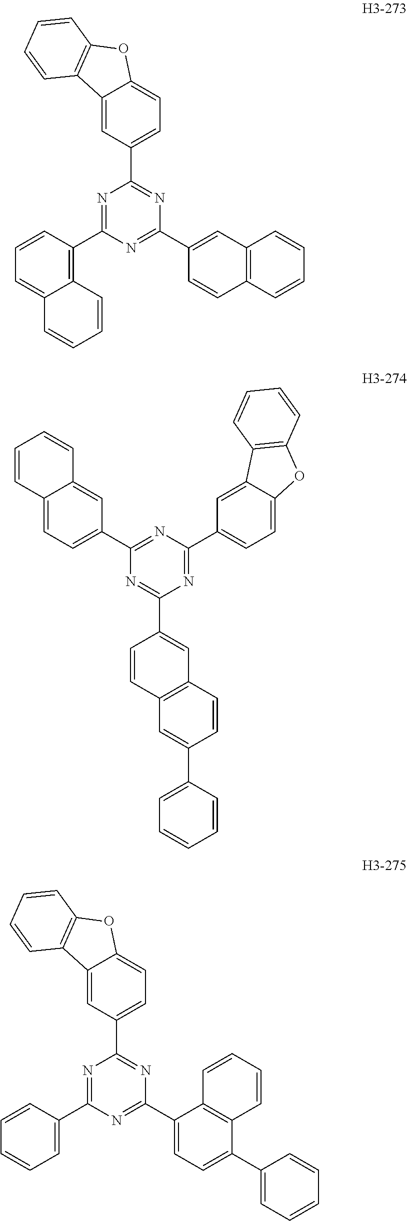 [SMILES: c1ccc2cc(-c3nc(-c4ccc5oc6ccccc6c5c4)nc(-c4cccc5ccccc45)n3)ccc2c1 ; c1ccc(-c2ccc3cc(-c4nc(-c5ccc6ccccc6c5)nc(-c5ccc6oc7ccccc7c6c5)n4)ccc3c2)cc1 ; c1ccc(-c2nc(-c3ccc4oc5ccccc5c4c3)nc(-c3ccc(-c4ccccc4)c4ccccc34)n2)cc1] 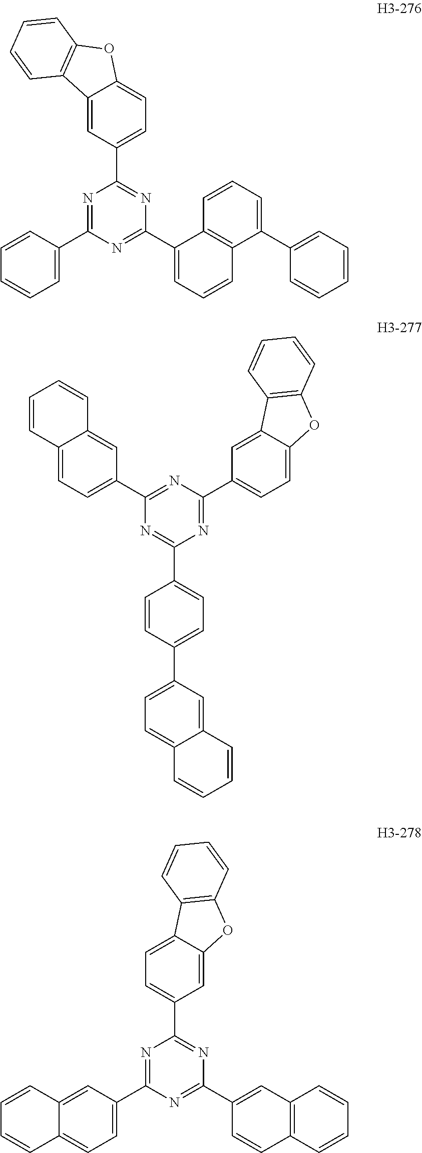 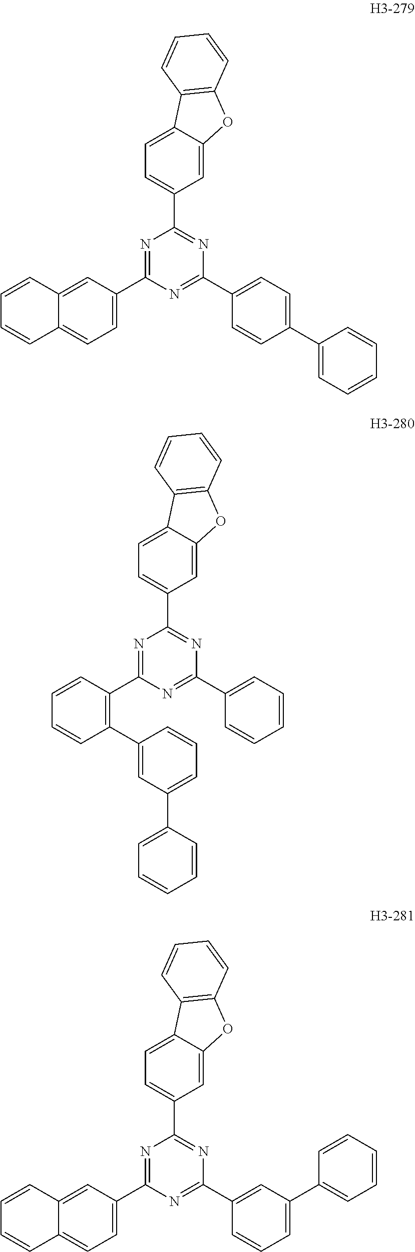 [SMILES: c1ccc(-c2ccc(-c3nc(-c4ccc5ccccc5c4)nc(-c4ccc5c(c4)oc4ccccc45)n3)cc2)cc1 ; c1ccc(-c2cccc(-c3ccccc3-c3nc(-c4ccccc4)nc(-c4ccc5c(c4)oc4ccccc45)n3)c2)cc1 ; c1ccc(-c2cccc(-c3nc(-c4ccc5ccccc5c4)nc(-c4ccc5c(c4)oc4ccccc45)n3)c2)cc1] 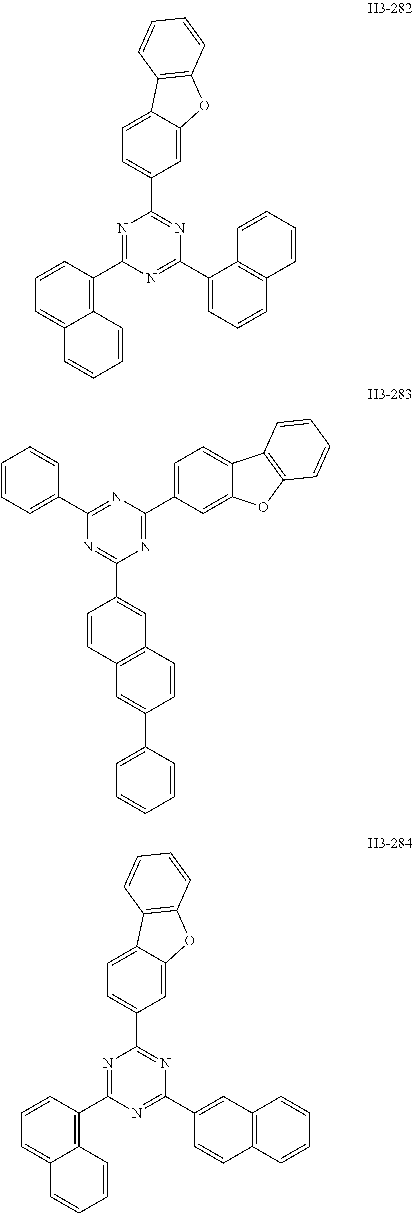 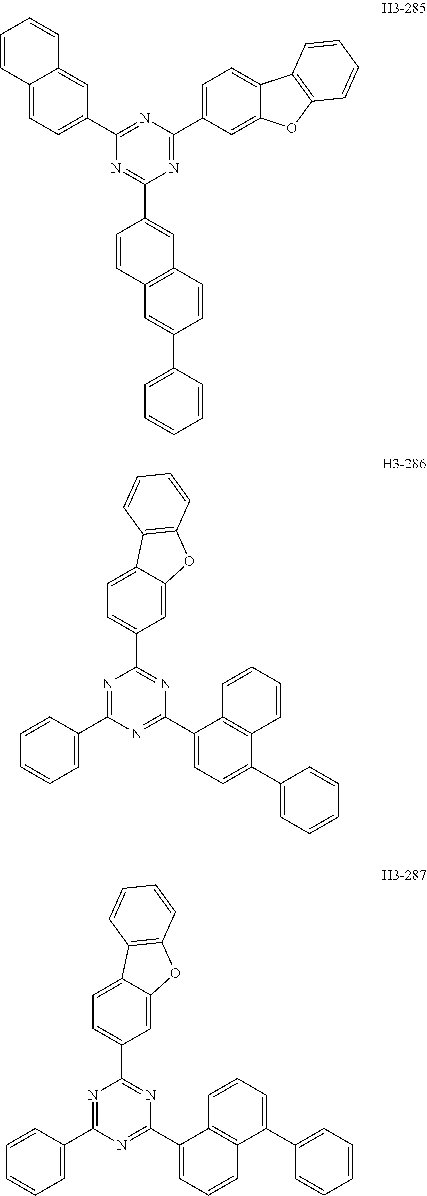 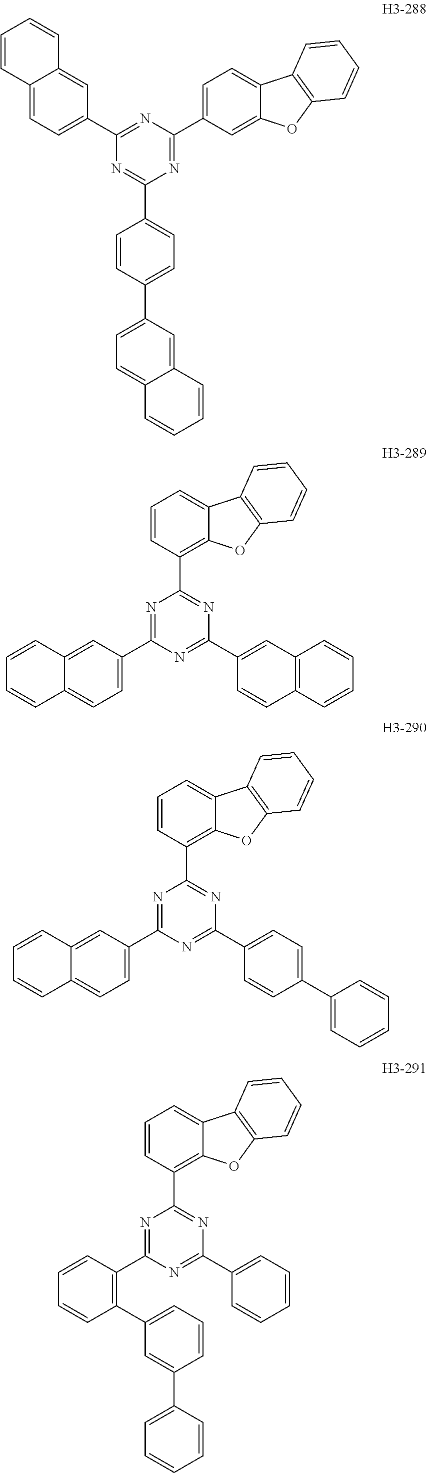 [SMILES: c1ccc2cc(-c3ccc(-c4nc(-c5ccc6ccccc6c5)nc(-c5ccc6c(c5)oc5ccccc56)n4)cc3)ccc2c1 ; c1ccc2cc(-c3nc(-c4ccc5ccccc5c4)nc(-c4cccc5c4oc4ccccc45)n3)ccc2c1 ; c1ccc(-c2ccc(-c3nc(-c4ccc5ccccc5c4)nc(-c4cccc5c4oc4ccccc45)n3)cc2)cc1 ; c1ccc(-c2cccc(-c3ccccc3-c3nc(-c4ccccc4)nc(-c4cccc5c4oc4ccccc45)n3)c2)cc1] 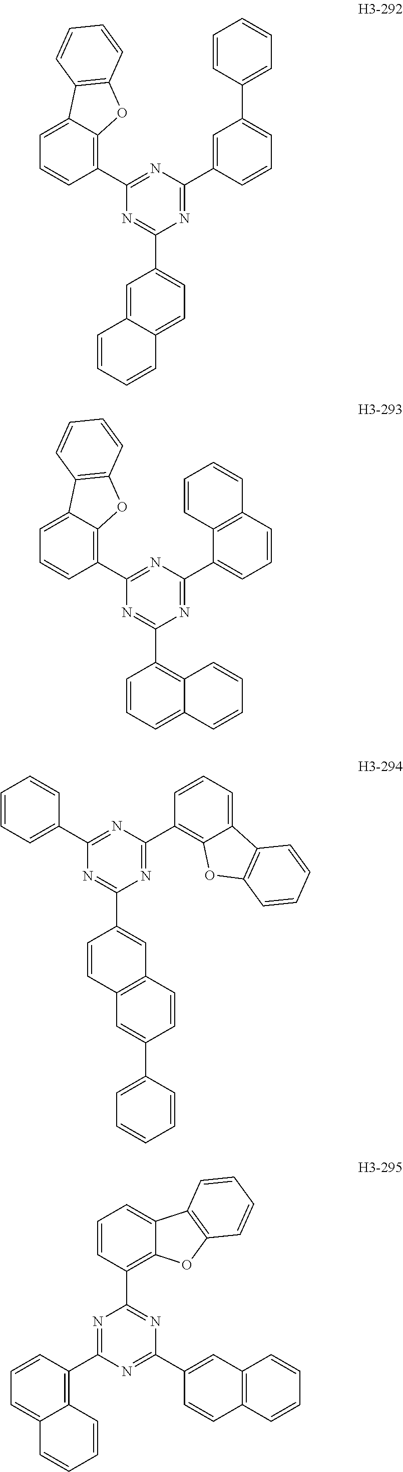 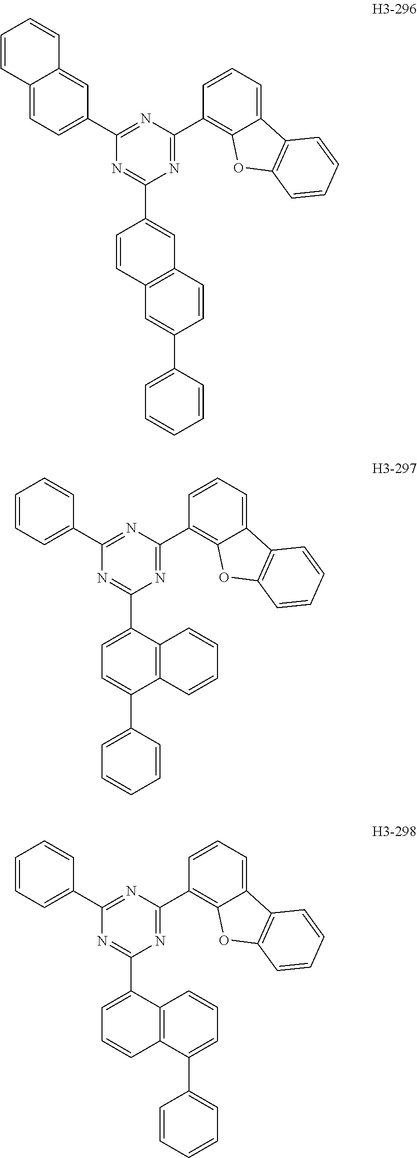 [SMILES: c1ccc(-c2ccc3cc(-c4nc(-c5ccc6ccccc6c5)nc(-c5cccc6c5oc5ccccc56)n4)ccc3c2)cc1 ; c1ccc(-c2nc(-c3ccc(-c4ccccc4)c4ccccc34)nc(-c3cccc4c3oc3ccccc34)n2)cc1 ; c1ccc(-c2nc(-c3cccc4c(-c5ccccc5)cccc34)nc(-c3cccc4c3oc3ccccc34)n2)cc1] 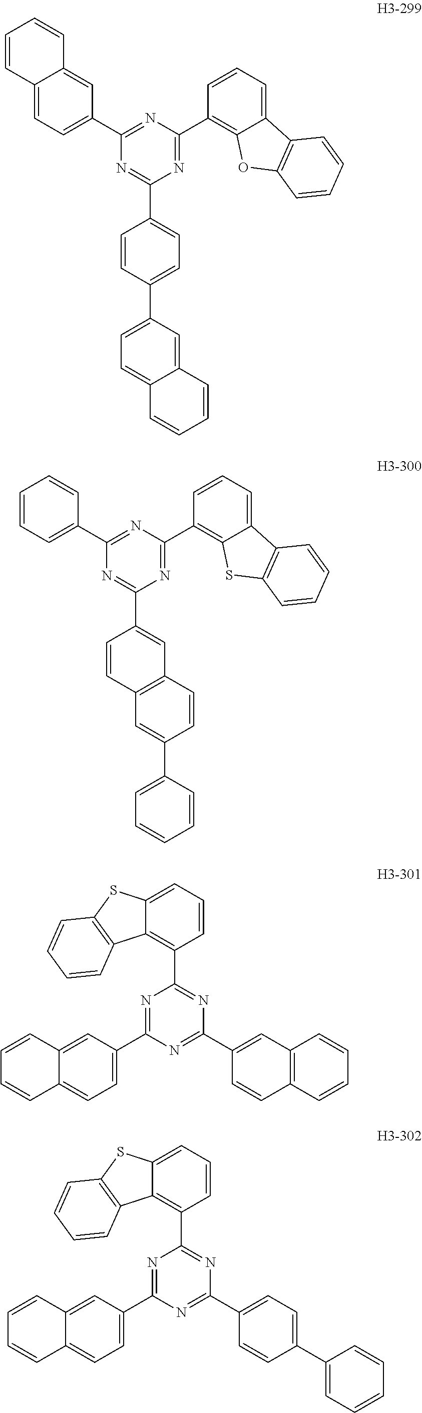 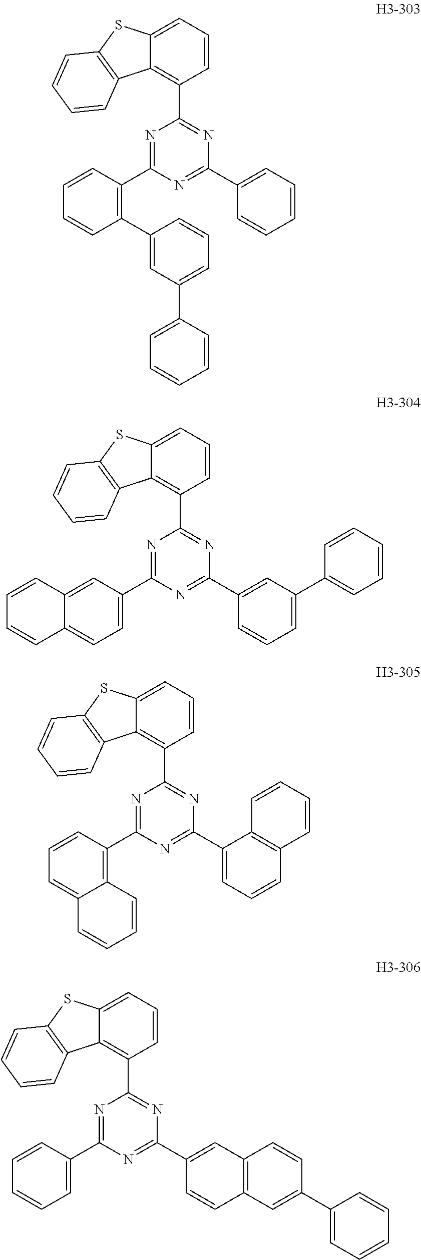 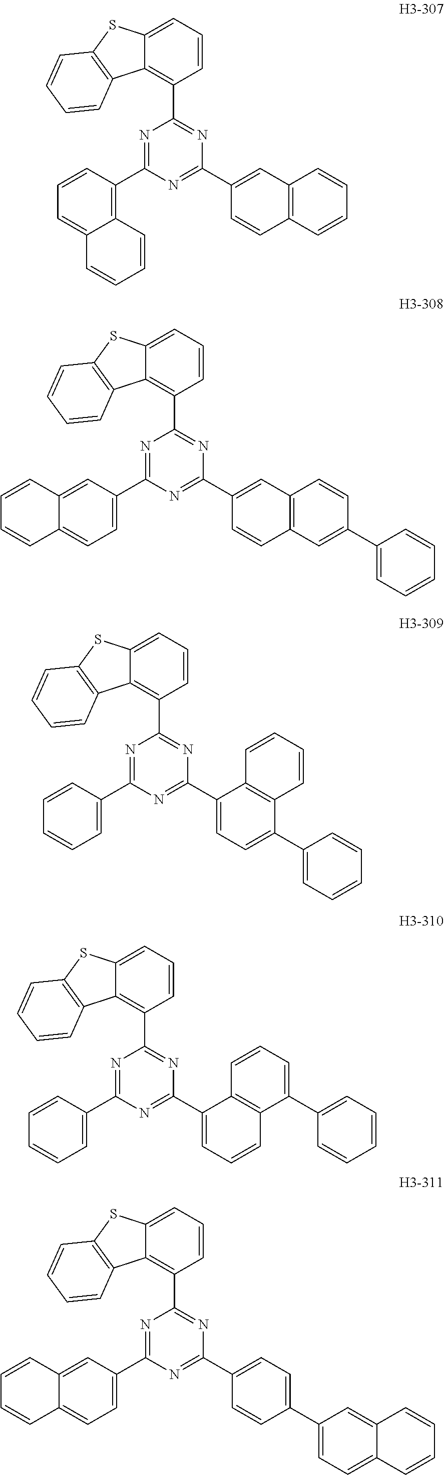 [SMILES: c1ccc2cc(-c3nc(-c4cccc5ccccc45)nc(-c4cccc5sc6ccccc6c45)n3)ccc2c1 ; c1ccc(-c2ccc3cc(-c4nc(-c5ccc6ccccc6c5)nc(-c5cccc6sc7ccccc7c56)n4)ccc3c2)cc1 ; c1ccc(-c2nc(-c3ccc(-c4ccccc4)c4ccccc34)nc(-c3cccc4sc5ccccc5c34)n2)cc1 ; c1ccc(-c2nc(-c3cccc4c(-c5ccccc5)cccc34)nc(-c3cccc4sc5ccccc5c34)n2)cc1 ; c1ccc2cc(-c3ccc(-c4nc(-c5ccc6ccccc6c5)nc(-c5cccc6sc7ccccc7c56)n4)cc3)ccc2c1] 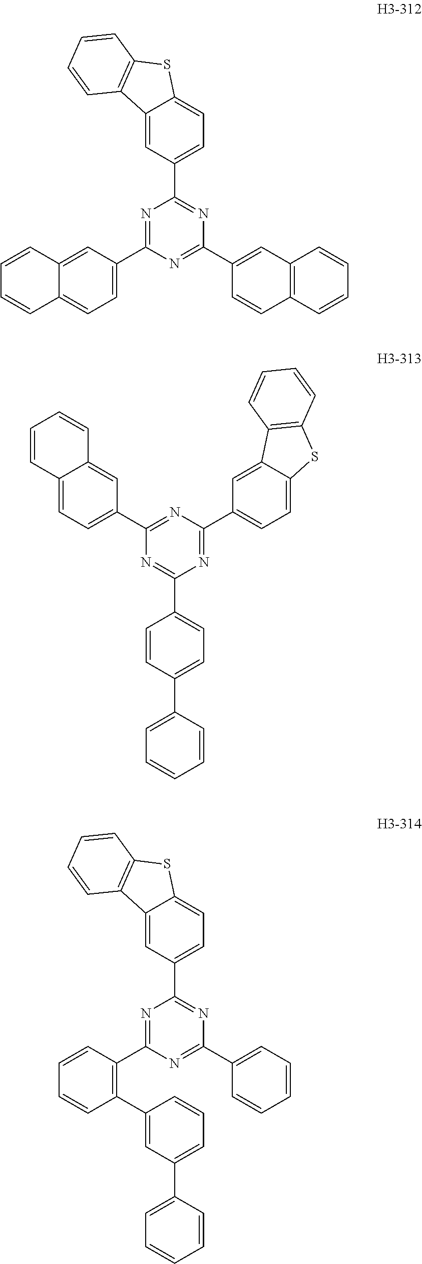 [SMILES: c1ccc2cc(-c3nc(-c4ccc5ccccc5c4)nc(-c4ccc5sc6ccccc6c5c4)n3)ccc2c1 ; c1ccc(-c2ccc(-c3nc(-c4ccc5ccccc5c4)nc(-c4ccc5sc6ccccc6c5c4)n3)cc2)cc1 ; c1ccc(-c2cccc(-c3ccccc3-c3nc(-c4ccccc4)nc(-c4ccc5sc6ccccc6c5c4)n3)c2)cc1] 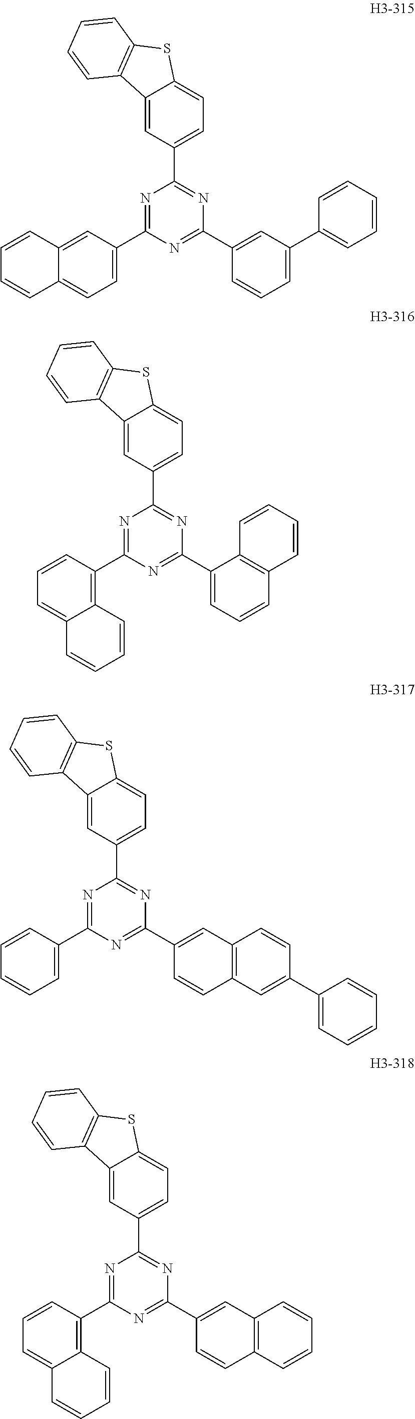 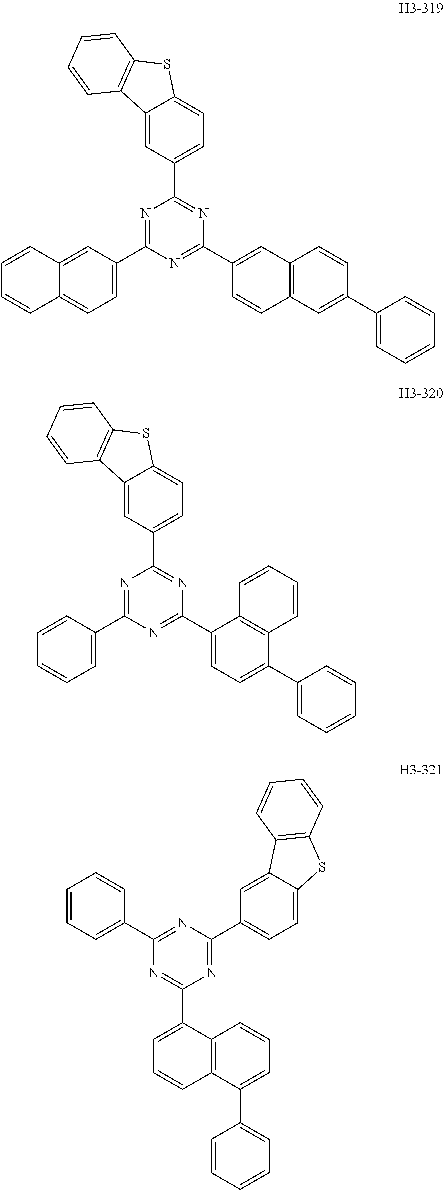 [SMILES: c1ccc(-c2ccc3cc(-c4nc(-c5ccc6ccccc6c5)nc(-c5ccc6sc7ccccc7c6c5)n4)ccc3c2)cc1 ; c1ccc(-c2nc(-c3ccc4sc5ccccc5c4c3)nc(-c3ccc(-c4ccccc4)c4ccccc34)n2)cc1 ; c1ccc(-c2nc(-c3ccc4sc5ccccc5c4c3)nc(-c3cccc4c(-c5ccccc5)cccc34)n2)cc1] 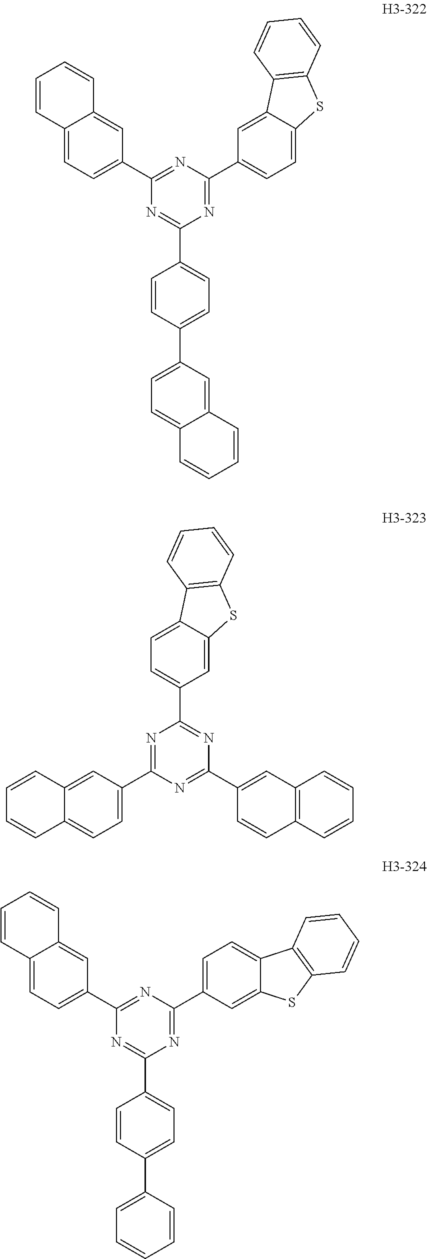 [SMILES: c1ccc2cc(-c3ccc(-c4nc(-c5ccc6ccccc6c5)nc(-c5ccc6sc7ccccc7c6c5)n4)cc3)ccc2c1 ; c1ccc2cc(-c3nc(-c4ccc5ccccc5c4)nc(-c4ccc5c(c4)sc4ccccc45)n3)ccc2c1 ; c1ccc(-c2ccc(-c3nc(-c4ccc5ccccc5c4)nc(-c4ccc5c(c4)sc4ccccc45)n3)cc2)cc1] 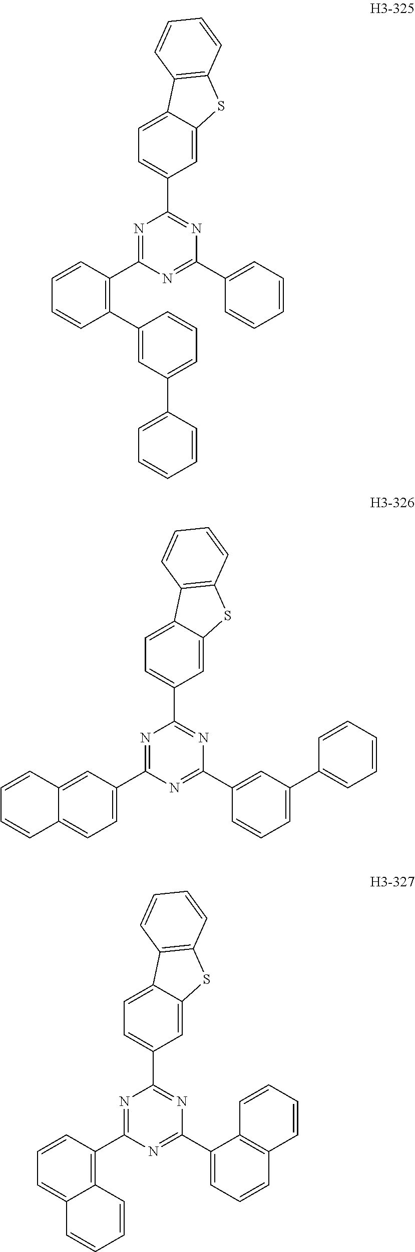 [SMILES: c1ccc(-c2cccc(-c3ccccc3-c3nc(-c4ccccc4)nc(-c4ccc5c(c4)sc4ccccc45)n3)c2)cc1 ; c1ccc(-c2cccc(-c3nc(-c4ccc5ccccc5c4)nc(-c4ccc5c(c4)sc4ccccc45)n3)c2)cc1 ; c1ccc2c(-c3nc(-c4ccc5c(c4)sc4ccccc45)nc(-c4cccc5ccccc45)n3)cccc2c1] 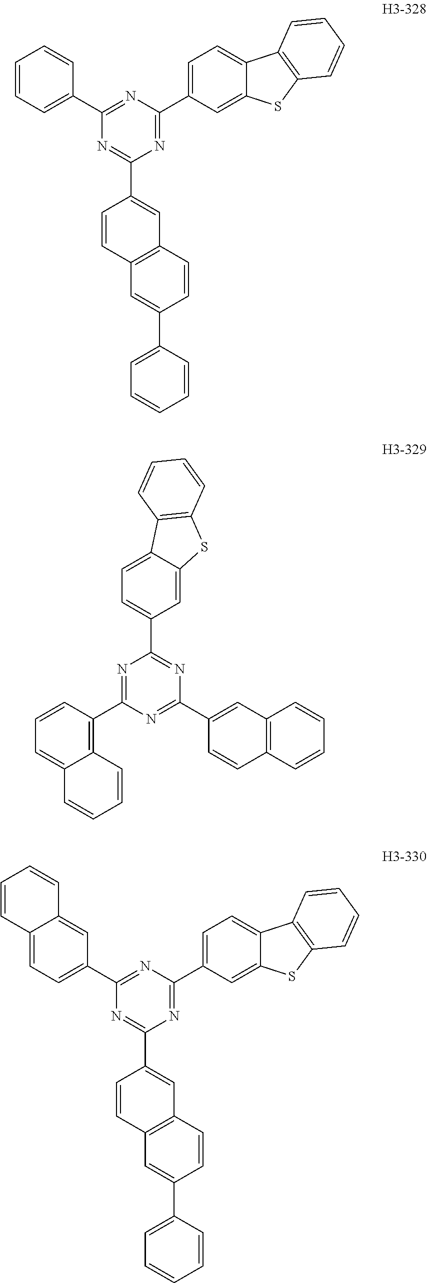 [SMILES: c1ccc(-c2ccc3cc(-c4nc(-c5ccccc5)nc(-c5ccc6c(c5)sc5ccccc56)n4)ccc3c2)cc1 ; c1ccc2cc(-c3nc(-c4ccc5c(c4)sc4ccccc45)nc(-c4cccc5ccccc45)n3)ccc2c1 ; c1ccc(-c2ccc3cc(-c4nc(-c5ccc6ccccc6c5)nc(-c5ccc6c(c5)sc5ccccc56)n4)ccc3c2)cc1] 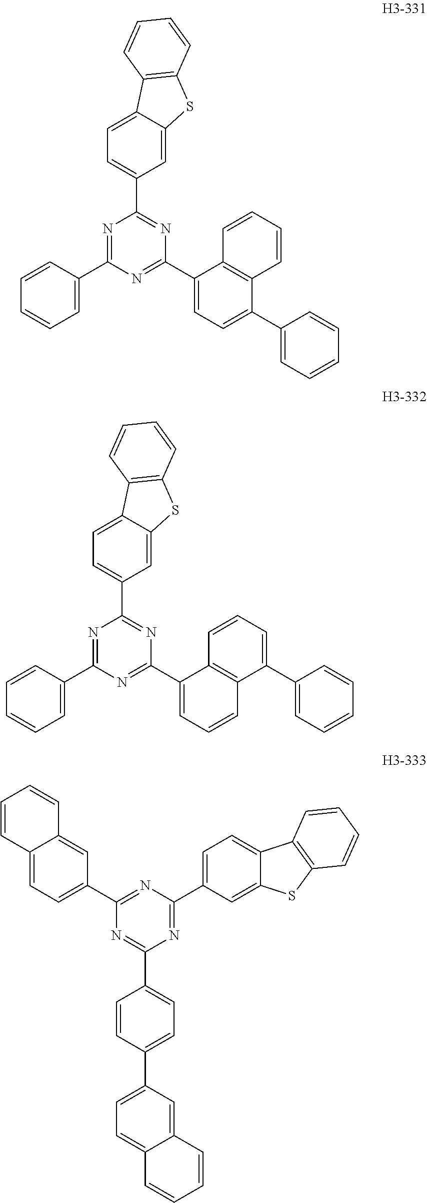 [SMILES: c1ccc(-c2nc(-c3ccc4c(c3)sc3ccccc34)nc(-c3ccc(-c4ccccc4)c4ccccc34)n2)cc1 ; c1ccc(-c2nc(-c3ccc4c(c3)sc3ccccc34)nc(-c3cccc4c(-c5ccccc5)cccc34)n2)cc1 ; c1ccc2cc(-c3ccc(-c4nc(-c5ccc6ccccc6c5)nc(-c5ccc6c(c5)sc5ccccc56)n4)cc3)ccc2c1] 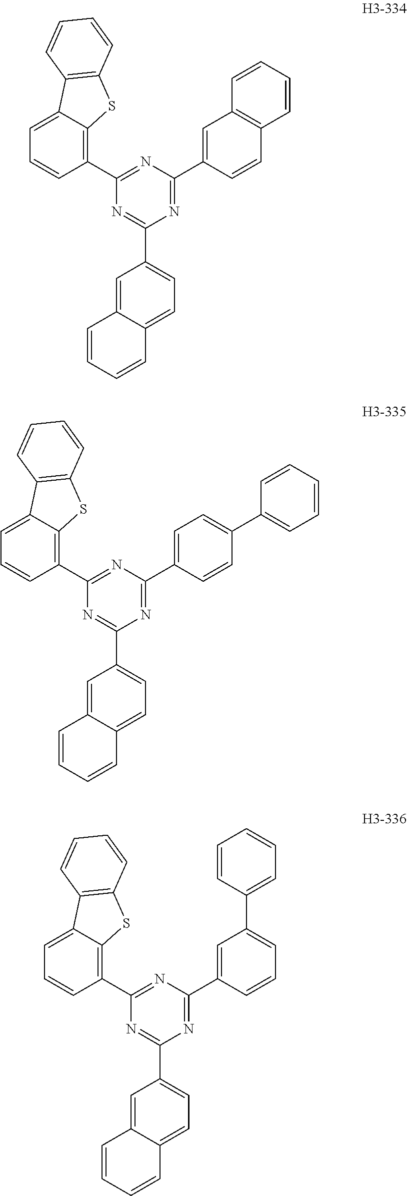 [SMILES: c1ccc2cc(-c3nc(-c4ccc5ccccc5c4)nc(-c4cccc5c4sc4ccccc45)n3)ccc2c1 ; c1ccc(-c2ccc(-c3nc(-c4ccc5ccccc5c4)nc(-c4cccc5c4sc4ccccc45)n3)cc2)cc1 ; c1ccc(-c2cccc(-c3nc(-c4ccc5ccccc5c4)nc(-c4cccc5c4sc4ccccc45)n3)c2)cc1] 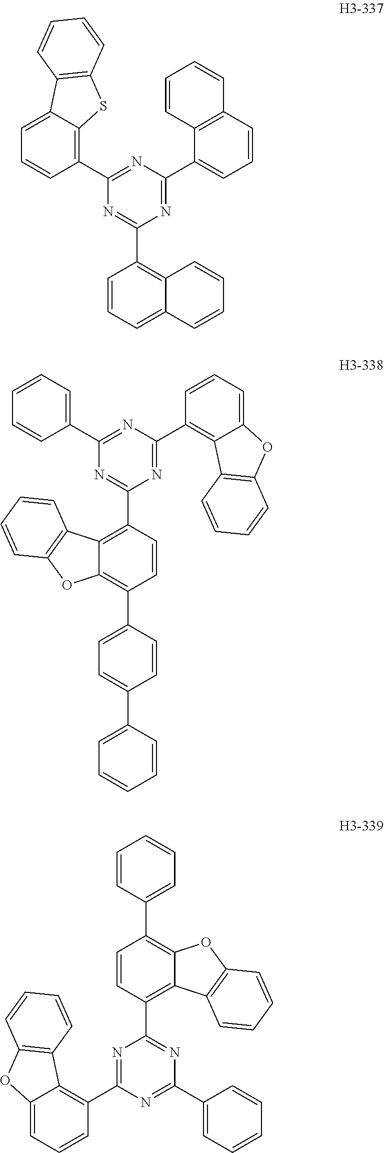 [SMILES: c1ccc2c(-c3nc(-c4cccc5ccccc45)nc(-c4cccc5c4sc4ccccc45)n3)cccc2c1 ; c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1] 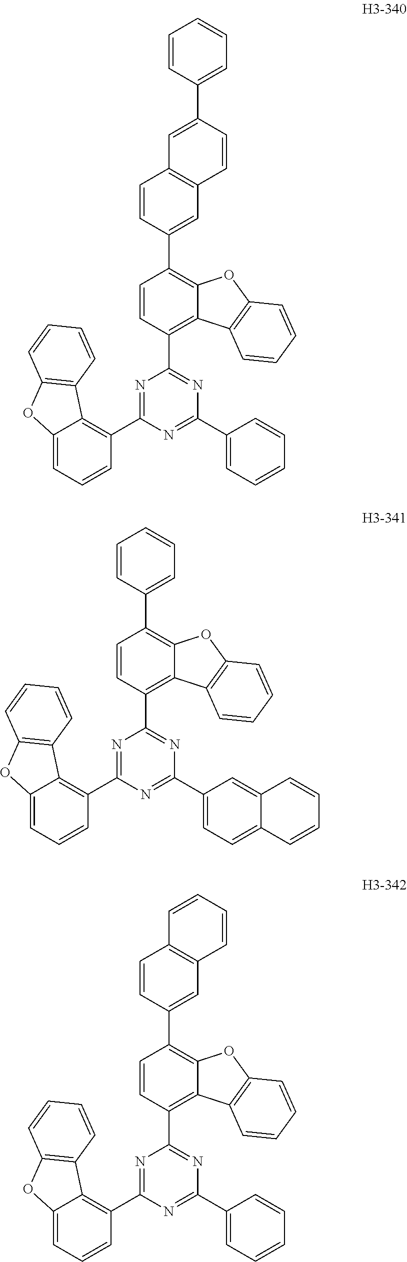 [SMILES: c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc(-c2ccc(-c3nc(-c4ccc5ccccc5c4)nc(-c4cccc5oc6ccccc6c45)n3)c3c2oc2ccccc23)cc1 ; c1ccc(-c2nc(-c3cccc4oc5ccccc5c34)nc(-c3ccc(-c4ccc5ccccc5c4)c4oc5ccccc5c34)n2)cc1] 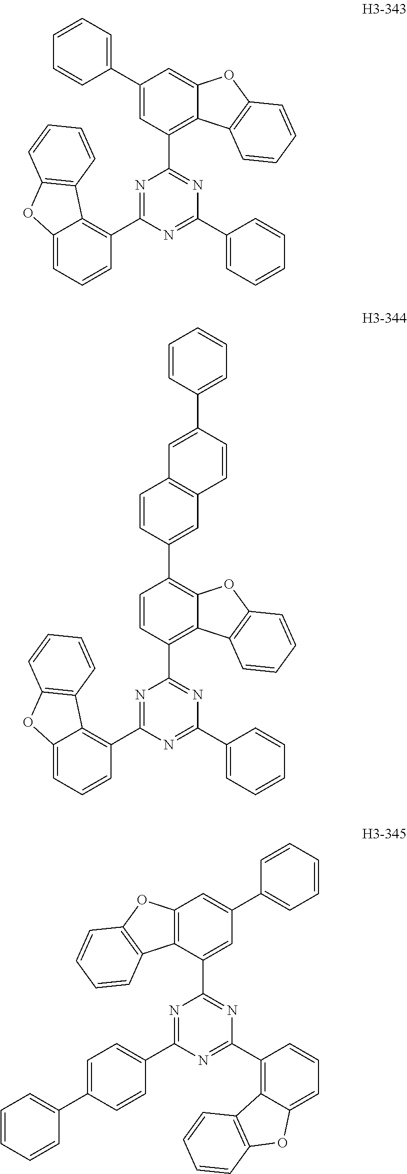 [SMILES: c1ccc(-c2cc(-c3nc(-c4ccccc4)nc(-c4cccc5oc6ccccc6c45)n3)c3c(c2)oc2ccccc23)cc1 ; c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc(-c2ccc(-c3nc(-c4cccc5oc6ccccc6c45)nc(-c4cc(-c5ccccc5)cc5oc6ccccc6c45)n3)cc2)cc1] 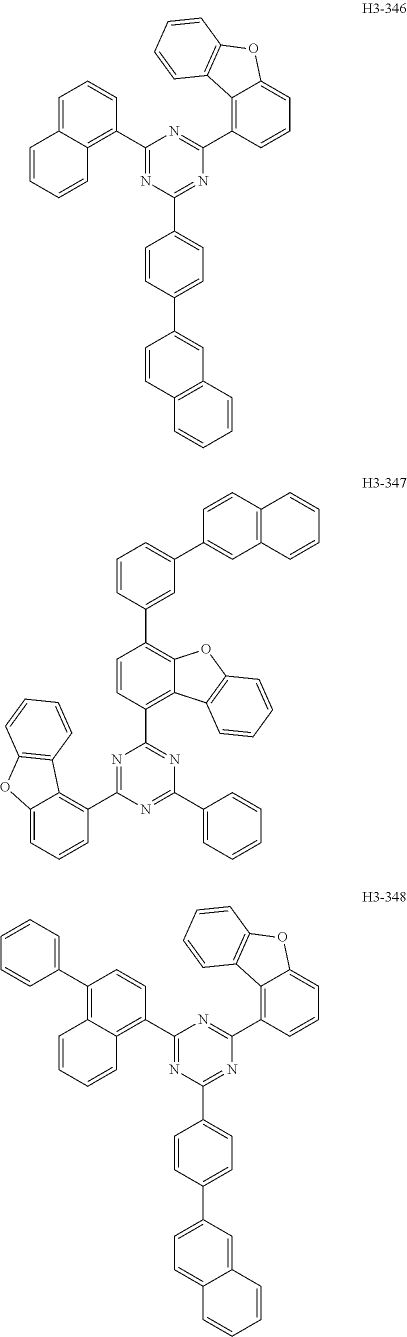 [SMILES: c1ccc2cc(-c3ccc(-c4nc(-c5cccc6ccccc56)nc(-c5cccc6oc7ccccc7c56)n4)cc3)ccc2c1 ; c1ccc(-c2nc(-c3cccc4oc5ccccc5c34)nc(-c3ccc(-c4cccc(-c5ccc6ccccc6c5)c4)c4oc5ccccc5c34)n2)cc1 ; c1ccc(-c2ccc(-c3nc(-c4ccc(-c5ccc6ccccc6c5)cc4)nc(-c4cccc5oc6ccccc6c45)n3)c3ccccc23)cc1] 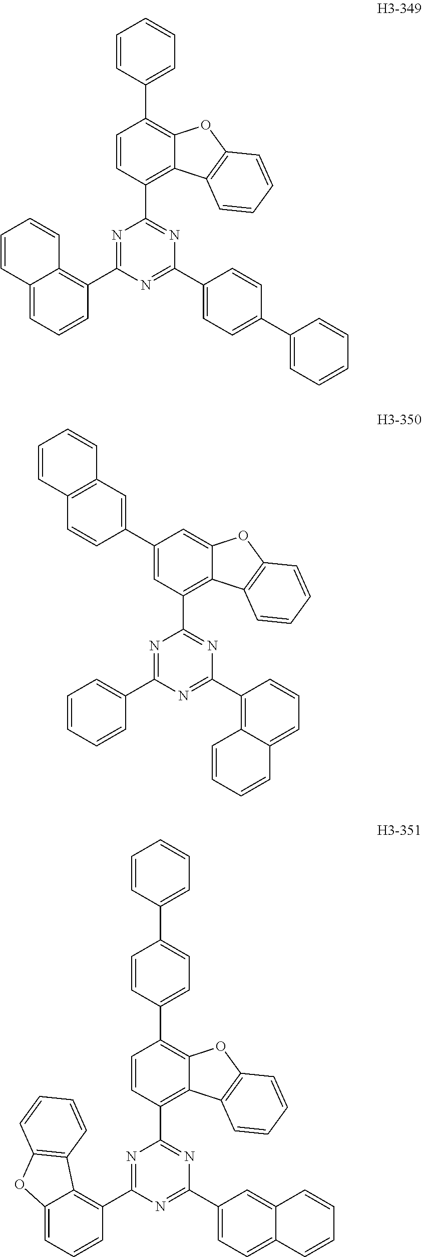 [SMILES: c1ccc(-c2ccc(-c3nc(-c4cccc5ccccc45)nc(-c4ccc(-c5ccccc5)c5oc6ccccc6c45)n3)cc2)cc1 ; c1ccc(-c2nc(-c3cccc4ccccc34)nc(-c3cc(-c4ccc5ccccc5c4)cc4oc5ccccc5c34)n2)cc1 ; c1ccc(-c2ccc(-c3ccc(-c4nc(-c5ccc6ccccc6c5)nc(-c5cccc6oc7ccccc7c56)n4)c4c3oc3ccccc34)cc2)cc1] 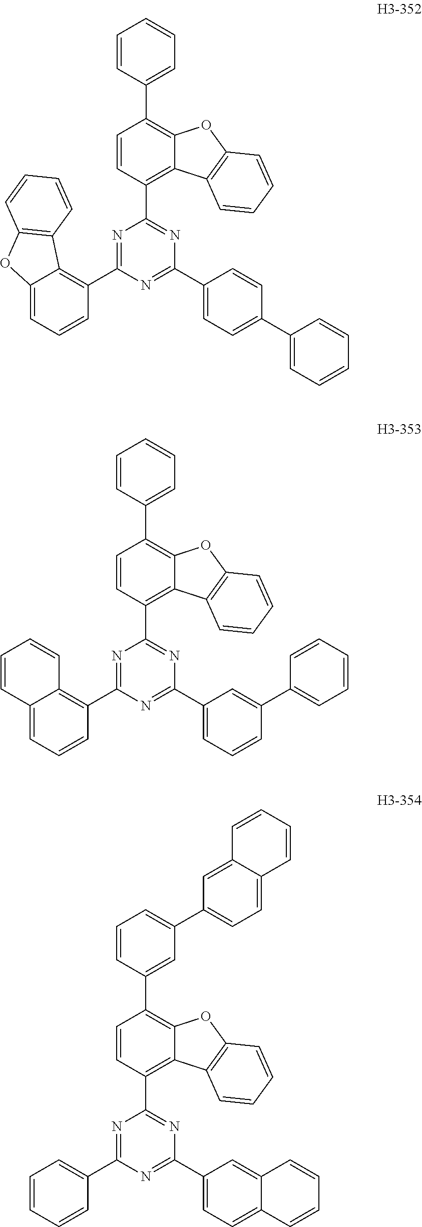 [SMILES: c1ccc(-c2ccc(-c3nc(-c4cccc5oc6ccccc6c45)nc(-c4ccc(-c5ccccc5)c5oc6ccccc6c45)n3)cc2)cc1 ; c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1] 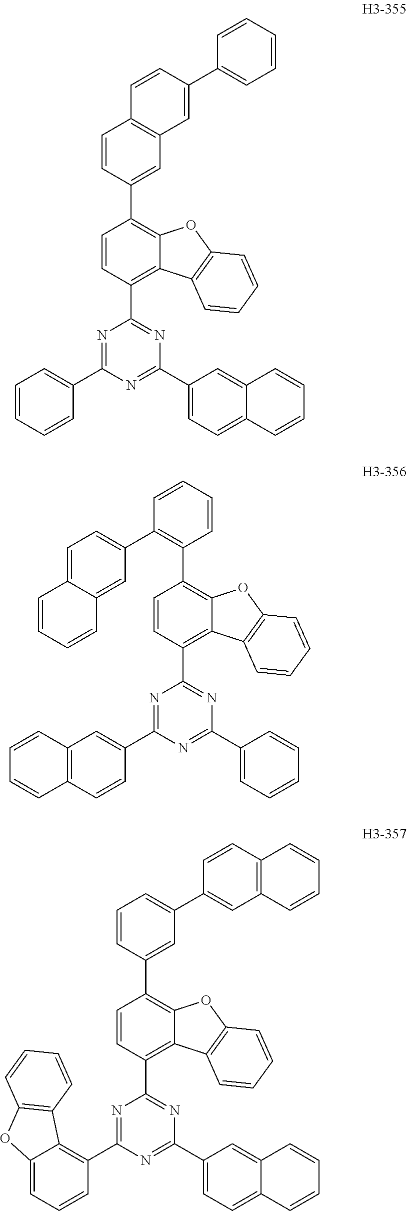 [SMILES: c1ccc(-c2ccc3ccc(-c4ccc(-c5nc(-c6ccccc6)nc(-c6ccc7ccccc7c6)n5)c5c4oc4ccccc45)cc3c2)cc1 ; c1ccc(-c2nc(-c3ccc4ccccc4c3)nc(-c3ccc(-c4ccccc4-c4ccc5ccccc5c4)c4oc5ccccc5c34)n2)cc1 ; c1cc(-c2ccc3ccccc3c2)cc(-c2ccc(-c3nc(-c4ccc5ccccc5c4)nc(-c4cccc5oc6ccccc6c45)n3)c3c2oc2ccccc23)c1] 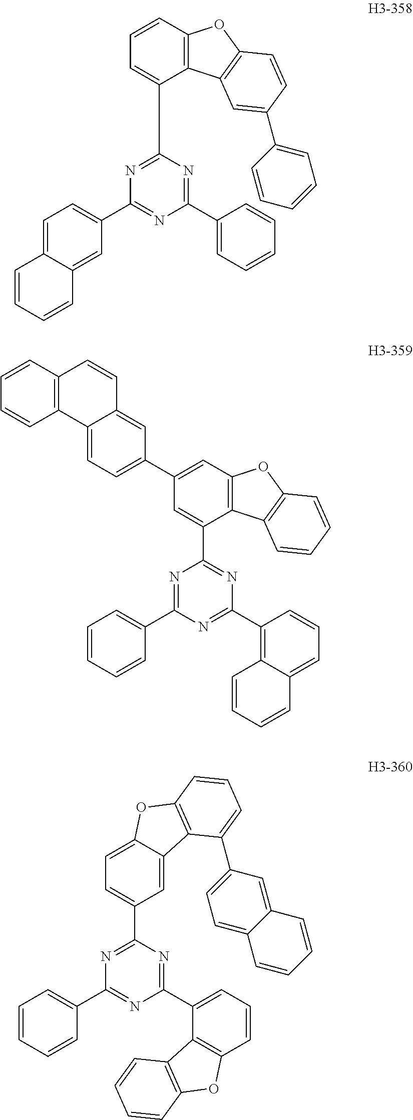 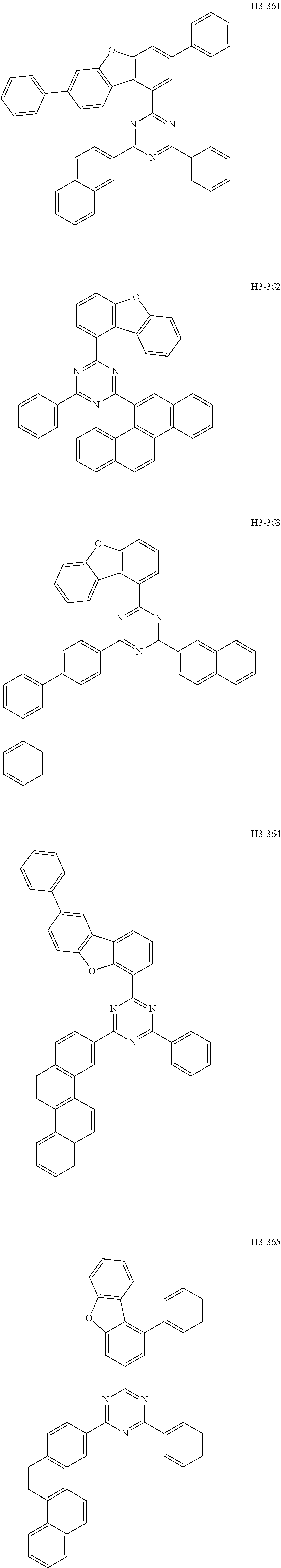 [SMILES: c1ccc(-c2ccc3c(c2)oc2cc(-c4ccccc4)cc(-c4nc(-c5ccccc5)nc(-c5ccc6ccccc6c5)n4)c23)cc1 ; c1ccc(-c2nc(-c3cccc4oc5ccccc5c34)nc(-c3cc4ccccc4c4ccc5ccccc5c34)n2)cc1 ; c1ccc(-c2cccc(-c3ccc(-c4nc(-c5ccc6ccccc6c5)nc(-c5cccc6oc7ccccc7c56)n4)cc3)c2)cc1 ; c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1] 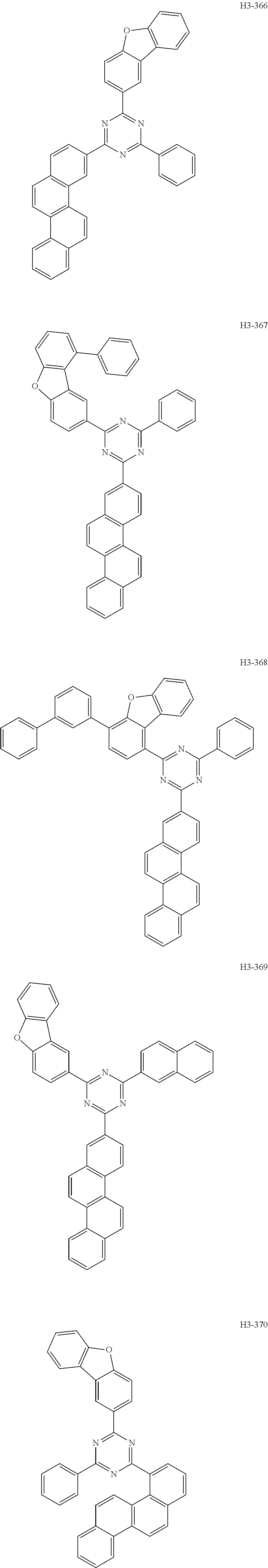 [SMILES: c1ccc(-c2nc(-c3ccc4ccc5c6ccccc6ccc5c4c3)nc(-c3ccc4oc5ccccc5c4c3)n2)cc1 ; c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4ccc5oc6ccccc6c5c4)n3)ccc2c1 ; c1ccc(-c2nc(-c3ccc4oc5ccccc5c4c3)nc(-c3cccc4ccc5c6ccccc6ccc5c34)n2)cc1] 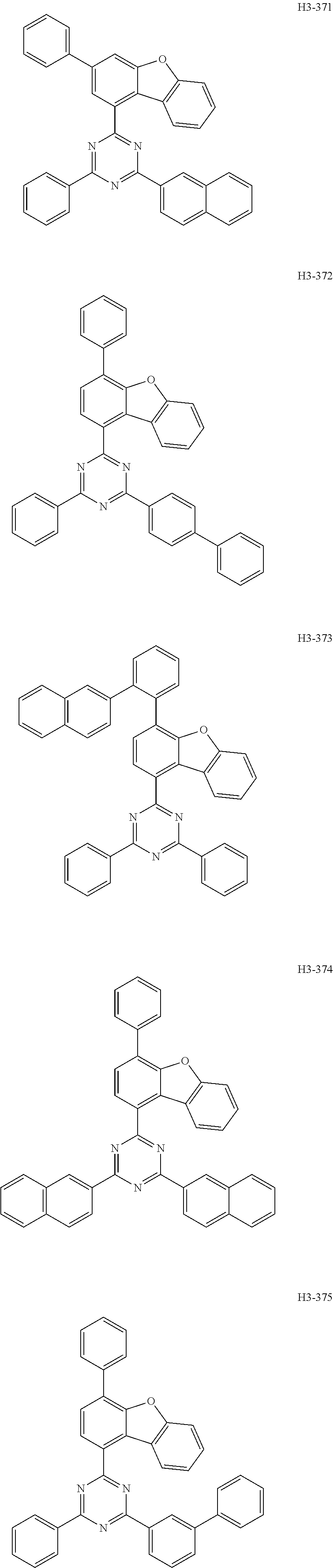 [SMILES: c1ccc(-c2cc(-c3nc(-c4ccccc4)nc(-c4ccc5ccccc5c4)n3)c3c(c2)oc2ccccc23)cc1 ; c1ccc(-c2ccc(-c3nc(-c4ccccc4)nc(-c4ccc(-c5ccccc5)c5oc6ccccc6c45)n3)cc2)cc1 ; c1ccc(-c2nc(-c3ccccc3)nc(-c3ccc(-c4ccccc4-c4ccc5ccccc5c4)c4oc5ccccc5c34)n2)cc1 ; c1ccc(-c2ccc(-c3nc(-c4ccc5ccccc5c4)nc(-c4ccc5ccccc5c4)n3)c3c2oc2ccccc23)cc1 ; c1ccc(-c2cccc(-c3nc(-c4ccccc4)nc(-c4ccc(-c5ccccc5)c5oc6ccccc6c45)n3)c2)cc1] 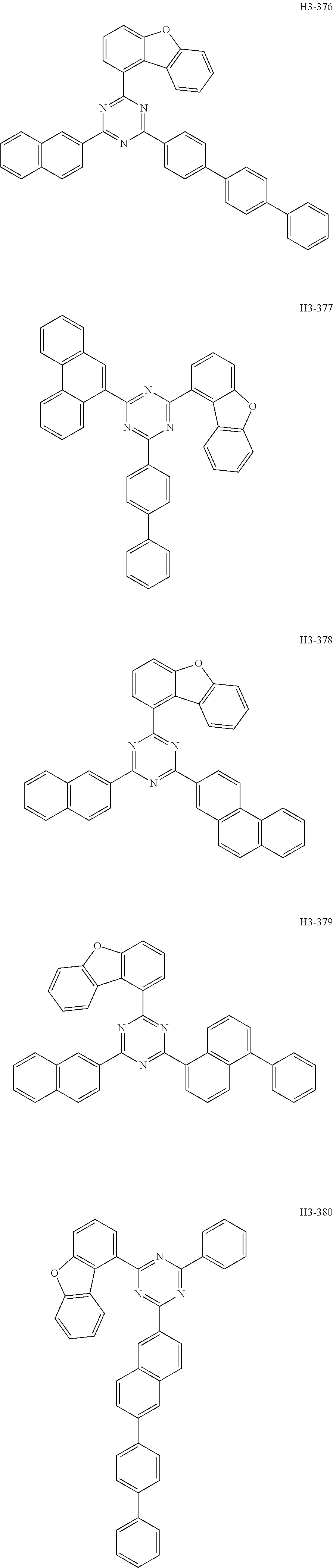 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3nc(-c4cc5ccccc5c5ccccc45)nc(-c4cccc5oc6ccccc6c45)n3)cc2)cc1 ; c1ccc2cc(-c3nc(-c4ccc5c(ccc6ccccc65)c4)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1 ; c1ccc(-c2ccc(-c3ccc4cc(-c5nc(-c6ccccc6)nc(-c6cccc7oc8ccccc8c67)n5)ccc4c3)cc2)cc1] 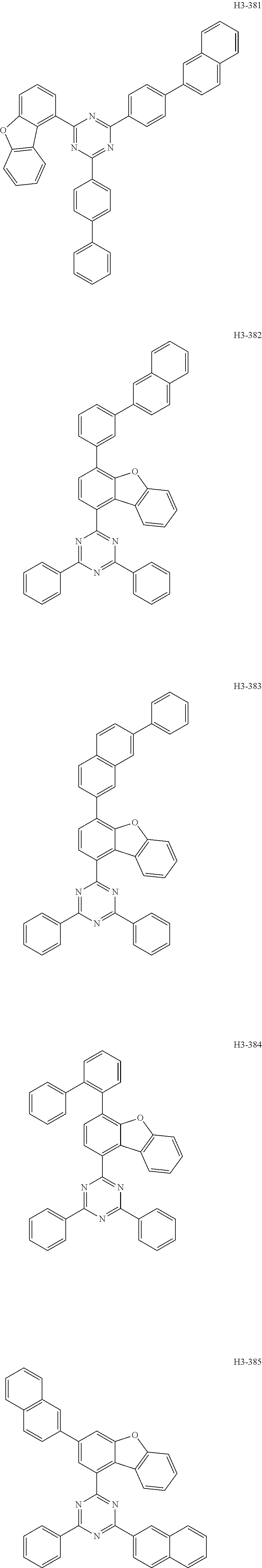 [SMILES: c1ccc(-c2ccc(-c3nc(-c4ccc(-c5ccc6ccccc6c5)cc4)nc(-c4cccc5oc6ccccc6c45)n3)cc2)cc1 ; c1ccc(-c2nc(-c3ccccc3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccccc6)n5)c5c4oc4ccccc45)cc3c2)cc1 ; c1ccc(-c2nc(-c3ccccc3)nc(-c3ccc(-c4ccccc4-c4ccccc4)c4oc5ccccc5c34)n2)cc1 ; c1ccc(-c2nc(-c3ccc4ccccc4c3)nc(-c3cc(-c4ccc5ccccc5c4)cc4oc5ccccc5c34)n2)cc1] 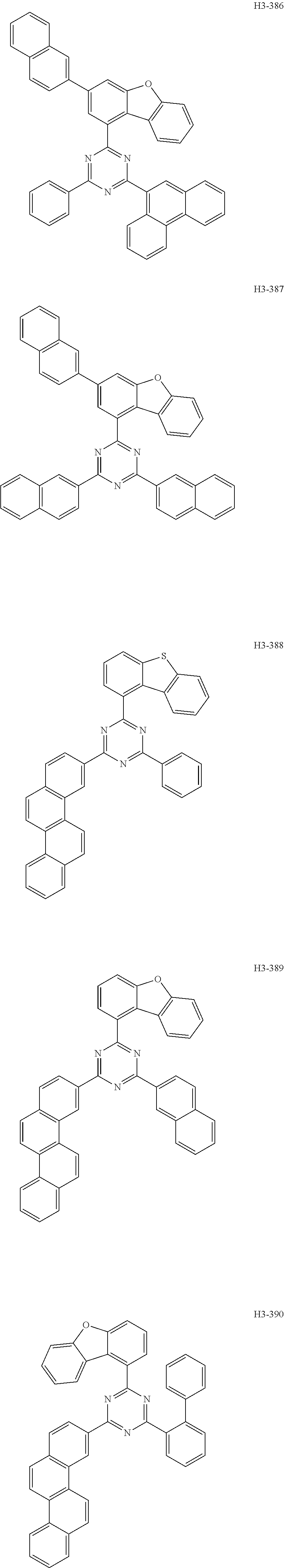 [SMILES: c1ccc(-c2nc(-c3cc4ccccc4c4ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5ccc6ccccc6c5)n4)c4c(c3)oc3ccccc34)ccc2c1 ; c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5oc6ccccc6c45)n3)ccc2c1 ; c1ccc(-c2ccccc2-c2nc(-c3ccc4ccc5c6ccccc6ccc5c4c3)nc(-c3cccc4oc5ccccc5c34)n2)cc1] 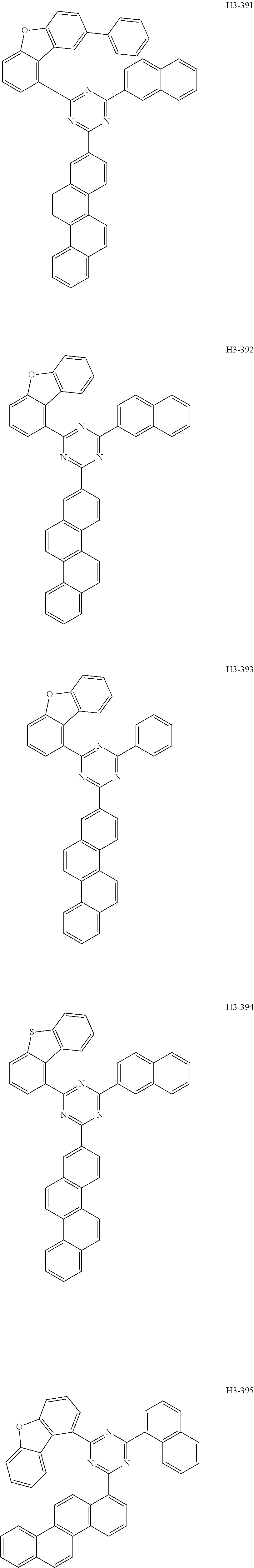 [SMILES: c1ccc(-c2ccc3oc4cccc(-c5nc(-c6ccc7ccccc7c6)nc(-c6ccc7c(ccc8c9ccccc9ccc78)c6)n5)c4c3c2)cc1 ; c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5sc6ccccc6c45)n3)ccc2c1 ; c1ccc2c(-c3nc(-c4cccc5c4ccc4c6ccccc6ccc54)nc(-c4cccc5oc6ccccc6c45)n3)cccc2c1] 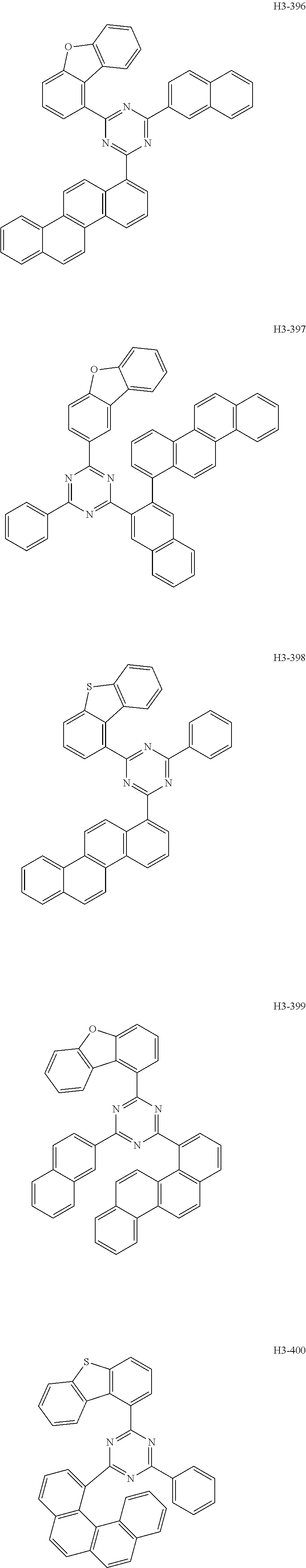 [SMILES: c1ccc2cc(-c3nc(-c4cccc5c4ccc4c6ccccc6ccc54)nc(-c4cccc5oc6ccccc6c45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4sc5ccccc5c34)n2)cc1 ; c1ccc2cc(-c3nc(-c4cccc5ccc6c7ccccc7ccc6c45)nc(-c4cccc5oc6ccccc6c45)n3)ccc2c1 ; c1ccc(-c2nc(-c3cccc4sc5ccccc5c34)nc(-c3cccc4ccc5ccc6ccccc6c5c34)n2)cc1] 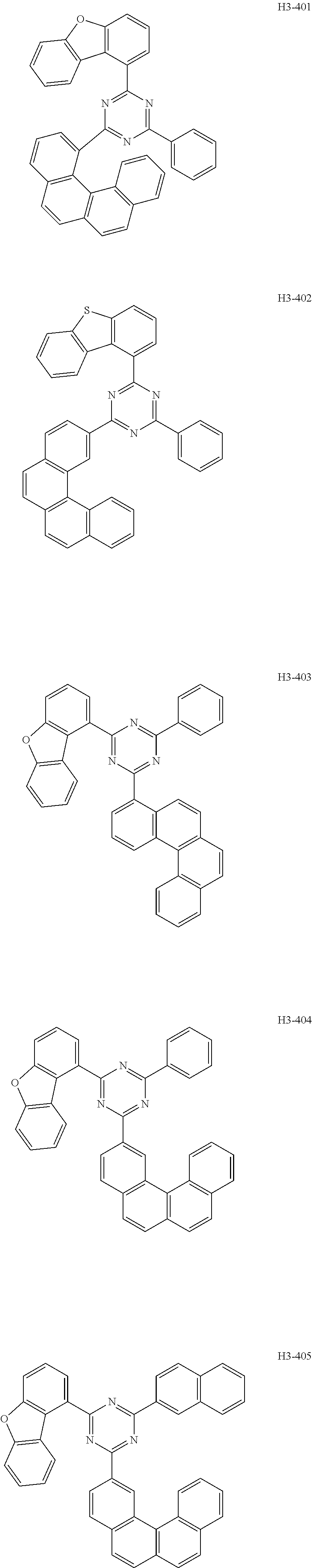 [SMILES: c1ccc(-c2nc(-c3cccc4oc5ccccc5c34)nc(-c3cccc4ccc5ccc6ccccc6c5c34)n2)cc1 ; c1ccc(-c2nc(-c3ccc4ccc5ccc6ccccc6c5c4c3)nc(-c3cccc4sc5ccccc5c34)n2)cc1 ; c1ccc(-c2nc(-c3cccc4c3ccc3ccc5ccccc5c34)nc(-c3cccc4oc5ccccc5c34)n2)cc1 ; c1ccc(-c2nc(-c3ccc4ccc5ccc6ccccc6c5c4c3)nc(-c3cccc4oc5ccccc5c34)n2)cc1 ; c1ccc2cc(-c3nc(-c4ccc5ccc6ccc7ccccc7c6c5c4)nc(-c4cccc5oc6ccccc6c45)n3)ccc2c1] 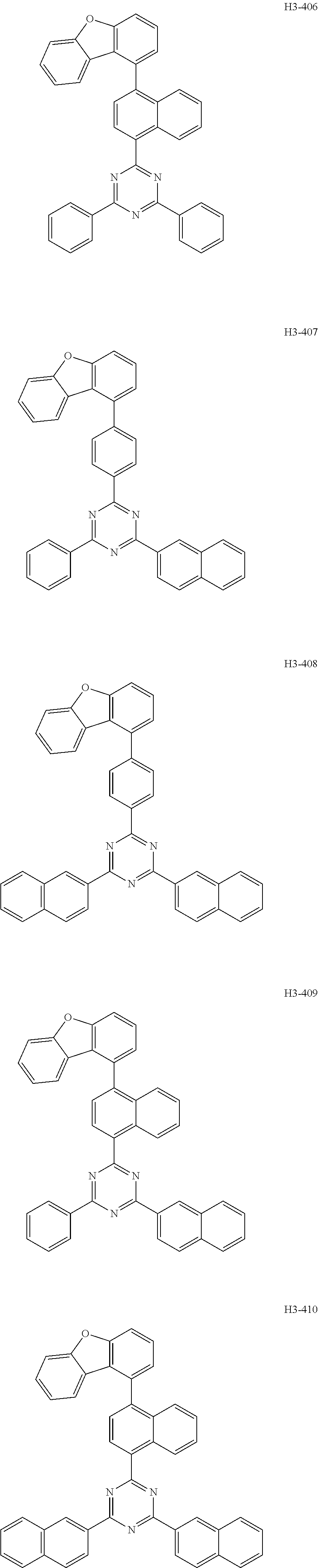 [SMILES: c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1 ; c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1] 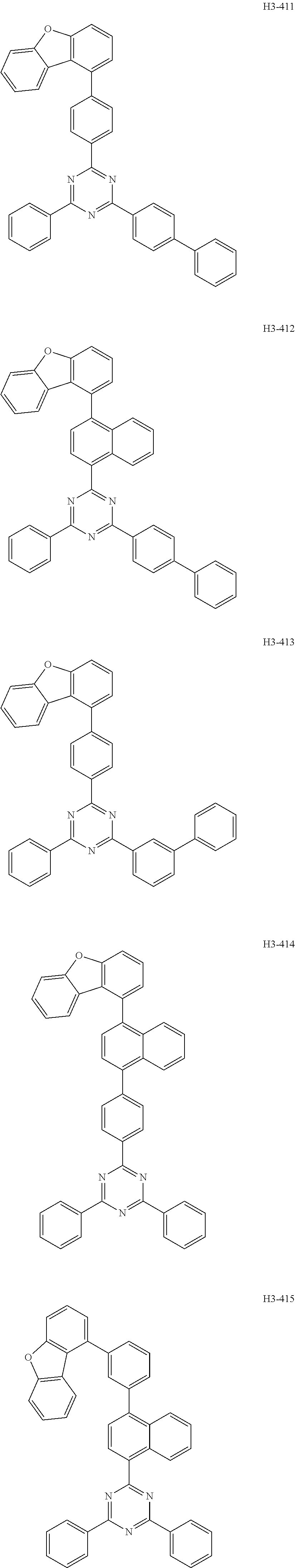 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1 ; c1ccc(-c2nc(-c3ccccc3)nc(-c3ccc(-c4ccc(-c5cccc6oc7ccccc7c56)c5ccccc45)cc3)n2)cc1 ; c1ccc(-c2nc(-c3ccccc3)nc(-c3ccc(-c4cccc(-c5cccc6oc7ccccc7c56)c4)c4ccccc34)n2)cc1] 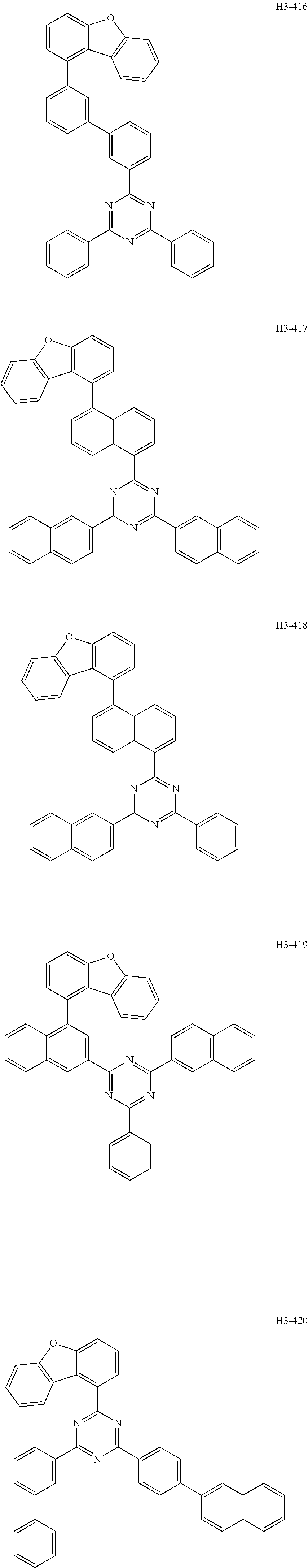 [SMILES: c1ccc(-c2nc(-c3ccccc3)nc(-c3cccc(-c4cccc(-c5cccc6oc7ccccc7c56)c4)c3)n2)cc1 ; c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1 ; c1ccc(-c2cccc(-c3nc(-c4ccc(-c5ccc6ccccc6c5)cc4)nc(-c4cccc5oc6ccccc6c45)n3)c2)cc1] 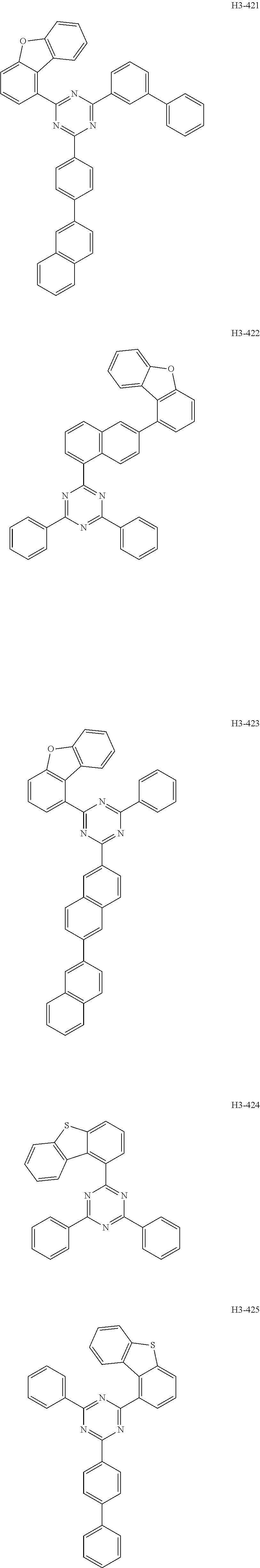 [SMILES: c1ccc(-c2cccc(-c3nc(-c4ccc(-c5ccc6ccccc6c5)cc4)nc(-c4cccc5oc6ccccc6c45)n3)c2)cc1 ; c1ccc(-c2nc(-c3ccccc3)nc(-c3cccc4cc(-c5cccc6oc7ccccc7c56)ccc34)n2)cc1 ; c1ccc(-c2nc(-c3ccc4cc(-c5ccc6ccccc6c5)ccc4c3)nc(-c3cccc4oc5ccccc5c34)n2)cc1 ; c1ccc(-c2nc(-c3ccccc3)nc(-c3cccc4sc5ccccc5c34)n2)cc1 ; c1ccc(-c2ccc(-c3nc(-c4ccccc4)nc(-c4cccc5sc6ccccc6c45)n3)cc2)cc1] 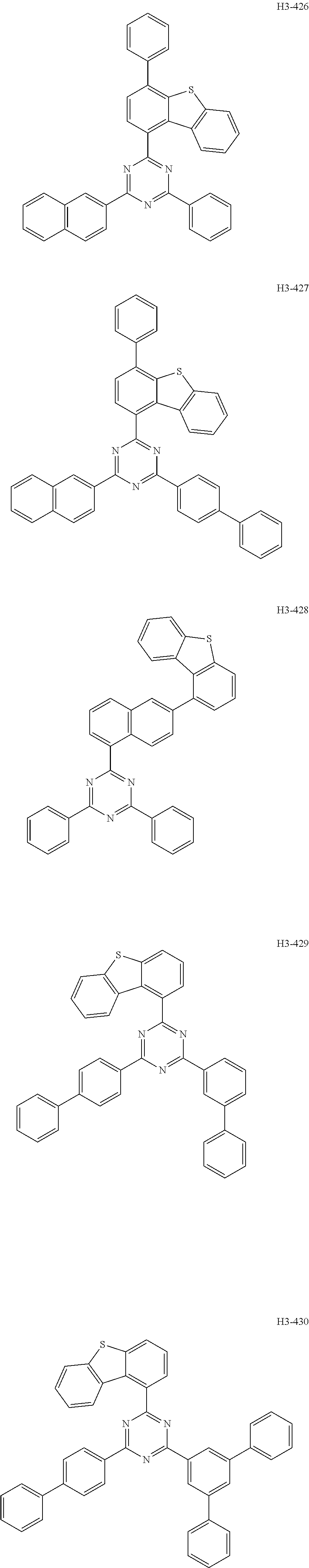 [SMILES: c1ccc(-c2nc(-c3ccc4ccccc4c3)nc(-c3ccc(-c4ccccc4)c4sc5ccccc5c34)n2)cc1 ; c1ccc(-c2ccc(-c3nc(-c4ccc5ccccc5c4)nc(-c4ccc(-c5ccccc5)c5sc6ccccc6c45)n3)cc2)cc1 ; c1ccc(-c2nc(-c3ccccc3)nc(-c3cccc4cc(-c5cccc6sc7ccccc7c56)ccc34)n2)cc1 ; c1ccc(-c2ccc(-c3nc(-c4cccc(-c5ccccc5)c4)nc(-c4cccc5sc6ccccc6c45)n3)cc2)cc1 ; c1ccc(-c2ccc(-c3nc(-c4cc(-c5ccccc5)cc(-c5ccccc5)c4)nc(-c4cccc5sc6ccccc6c45)n3)cc2)cc1] 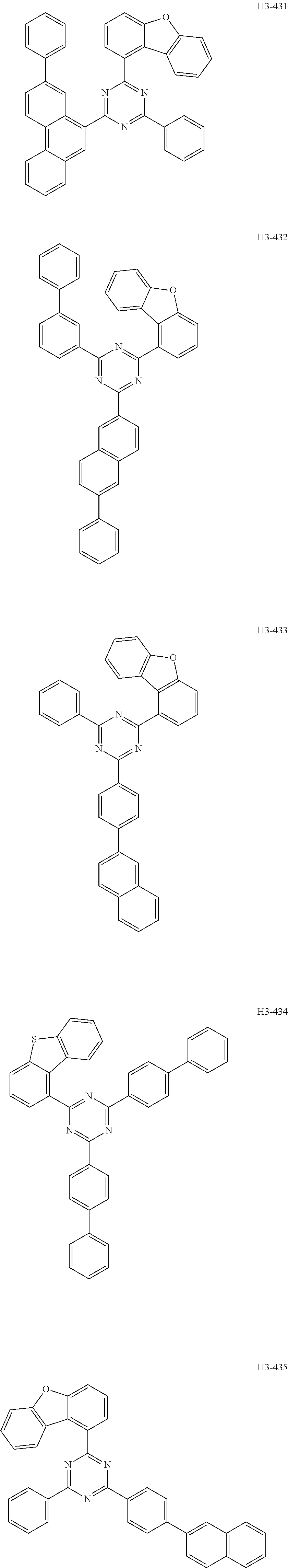 [SMILES: c1ccc(-c2ccc3c(c2)c(-c2nc(-c4ccccc4)nc(-c4cccc5oc6ccccc6c45)n2)cc2ccccc23)cc1 ; c1ccc(-c2cccc(-c3nc(-c4ccc5cc(-c6ccccc6)ccc5c4)nc(-c4cccc5oc6ccccc6c45)n3)c2)cc1 ; c1ccc(-c2nc(-c3ccc(-c4ccc5ccccc5c4)cc3)nc(-c3cccc4oc5ccccc5c34)n2)cc1 ; c1ccc(-c2ccc(-c3nc(-c4ccc(-c5ccccc5)cc4)nc(-c4cccc5sc6ccccc6c45)n3)cc2)cc1 ; c1ccc(-c2nc(-c3ccc(-c4ccc5ccccc5c4)cc3)nc(-c3cccc4oc5ccccc5c34)n2)cc1] 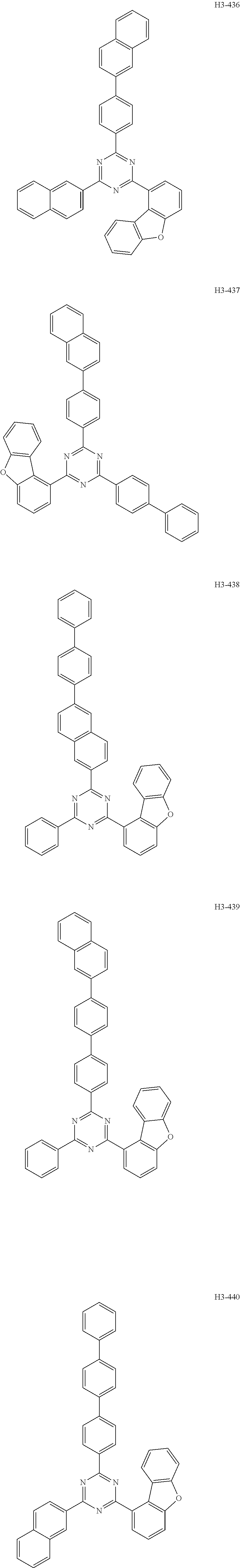 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccc(-c5ccc6ccccc6c5)cc4)nc(-c4cccc5oc6ccccc6c45)n3)cc2)cc1 ; c1ccc(-c2ccc(-c3ccc4cc(-c5nc(-c6ccccc6)nc(-c6cccc7oc8ccccc8c67)n5)ccc4c3)cc2)cc1 ; c1ccc(-c2nc(-c3ccc(-c4ccc(-c5ccc6ccccc6c5)cc4)cc3)nc(-c3cccc4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1] 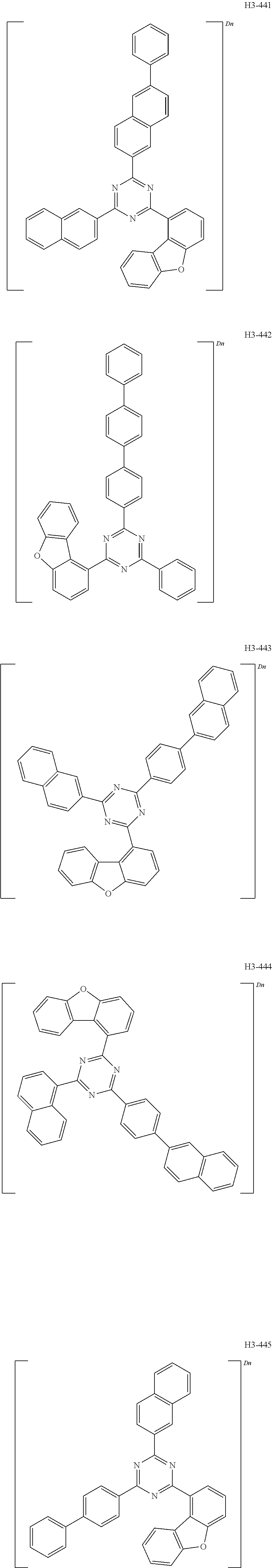 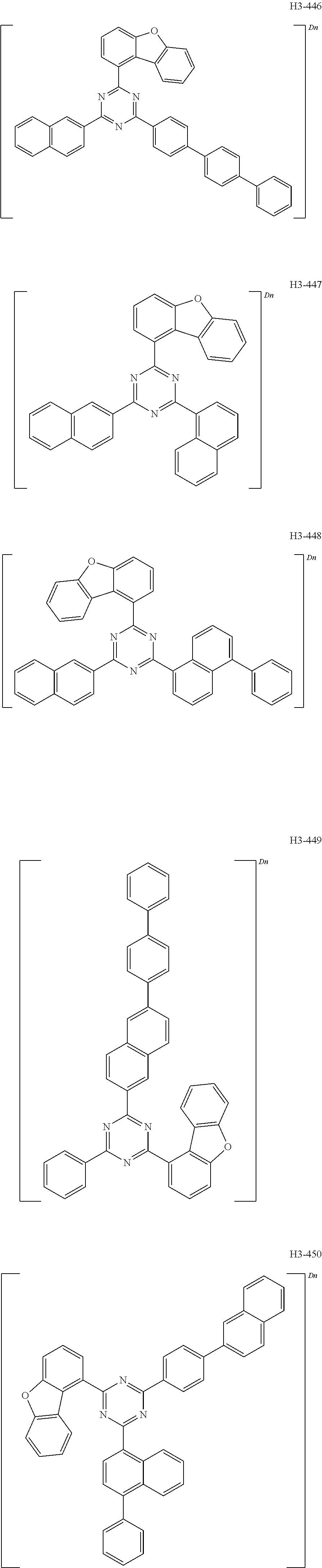 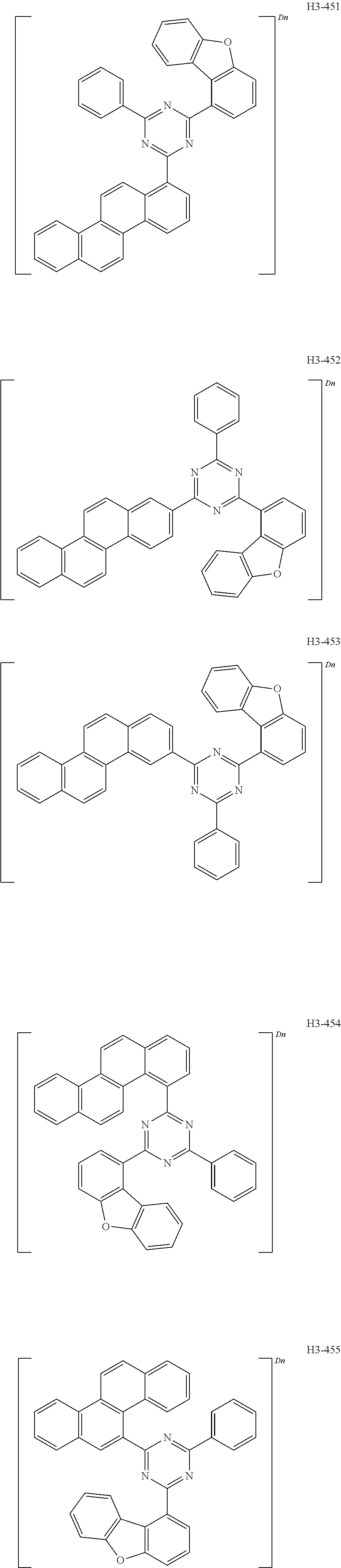 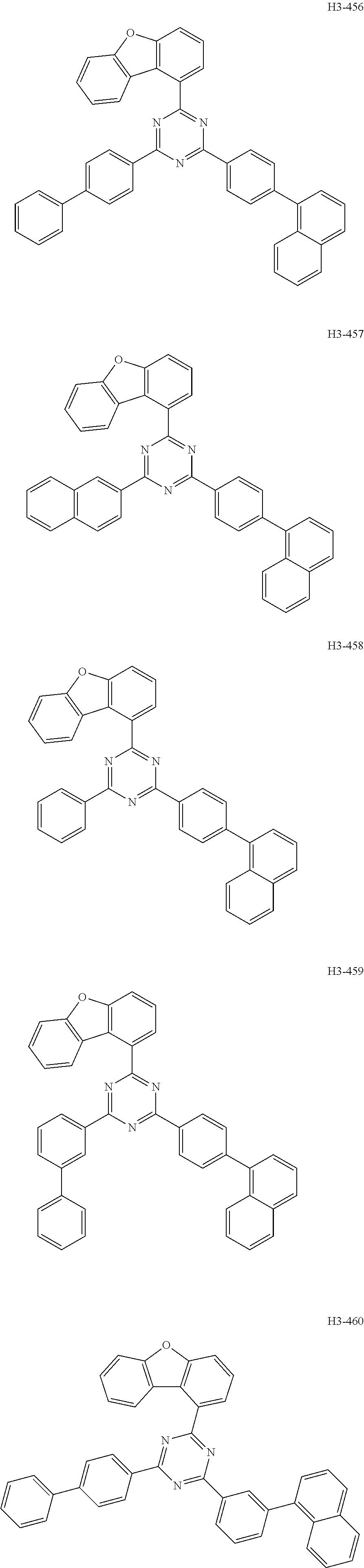 [SMILES: c1ccc(-c2ccc(-c3nc(-c4ccc(-c5cccc6ccccc56)cc4)nc(-c4cccc5oc6ccccc6c45)n3)cc2)cc1 ; c1ccc2cc(-c3nc(-c4ccc(-c5cccc6ccccc56)cc4)nc(-c4cccc5oc6ccccc6c45)n3)ccc2c1 ; c1ccc(-c2nc(-c3ccc(-c4cccc5ccccc45)cc3)nc(-c3cccc4oc5ccccc5c34)n2)cc1 ; c1ccc(-c2cccc(-c3nc(-c4ccc(-c5cccc6ccccc56)cc4)nc(-c4cccc5oc6ccccc6c45)n3)c2)cc1 ; c1ccc(-c2ccc(-c3nc(-c4cccc(-c5cccc6ccccc56)c4)nc(-c4cccc5oc6ccccc6c45)n3)cc2)cc1] 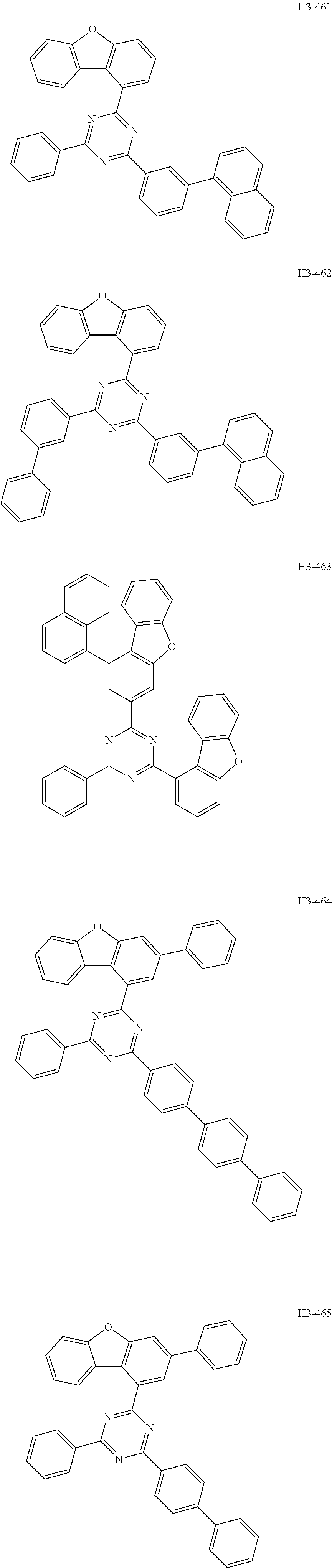 [SMILES: c1ccc(-c2nc(-c3cccc(-c4cccc5ccccc45)c3)nc(-c3cccc4oc5ccccc5c34)n2)cc1 ; c1ccc(-c2cccc(-c3nc(-c4cccc(-c5cccc6ccccc56)c4)nc(-c4cccc5oc6ccccc6c45)n3)c2)cc1 ; c1ccc(-c2nc(-c3cc(-c4cccc5ccccc45)c4c(c3)oc3ccccc34)nc(-c3cccc4oc5ccccc5c34)n2)cc1 ; c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3nc(-c4ccccc4)nc(-c4cc(-c5ccccc5)cc5oc6ccccc6c45)n3)cc2)cc1] 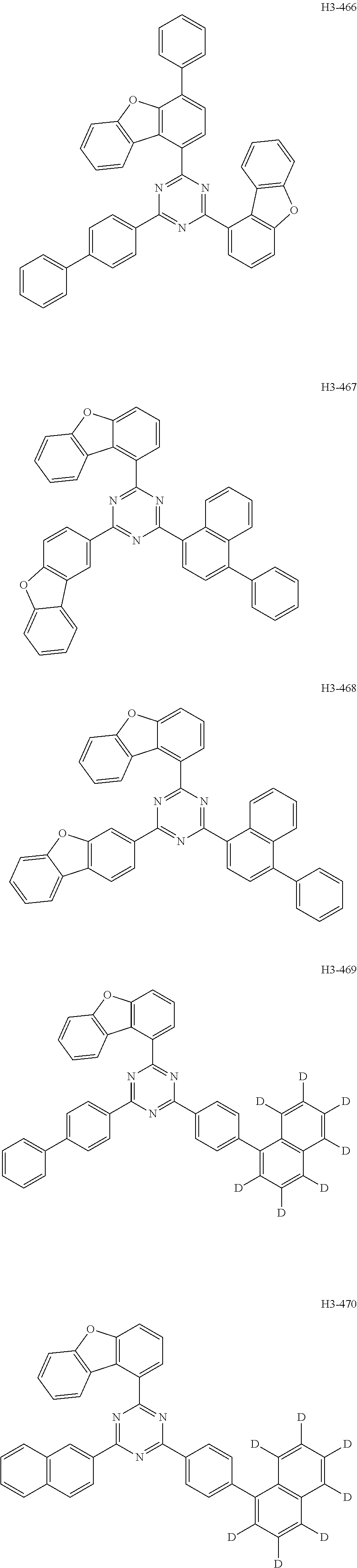 [SMILES: c1ccc(-c2ccc(-c3nc(-c4cccc5oc6ccccc6c45)nc(-c4ccc(-c5ccccc5)c5oc6ccccc6c45)n3)cc2)cc1 ; c1ccc(-c2ccc(-c3nc(-c4ccc5oc6ccccc6c5c4)nc(-c4cccc5oc6ccccc6c45)n3)c3ccccc23)cc1 ; c1ccc(-c2ccc(-c3nc(-c4ccc5c(c4)oc4ccccc45)nc(-c4cccc5oc6ccccc6c45)n3)c3ccccc23)cc1 ; [2H]c1c([2H])c([2H])c2c(-c3ccc(-c4nc(-c5ccc(-c6ccccc6)cc5)nc(-c5cccc6oc7ccccc7c56)n4)cc3)c([2H])c([2H])c([2H])c2c1[2H] ; [2H]c1c([2H])c([2H])c2c(-c3ccc(-c4nc(-c5ccc6ccccc6c5)nc(-c5cccc6oc7ccccc7c56)n4)cc3)c([2H])c([2H])c([2H])c2c1[2H]] 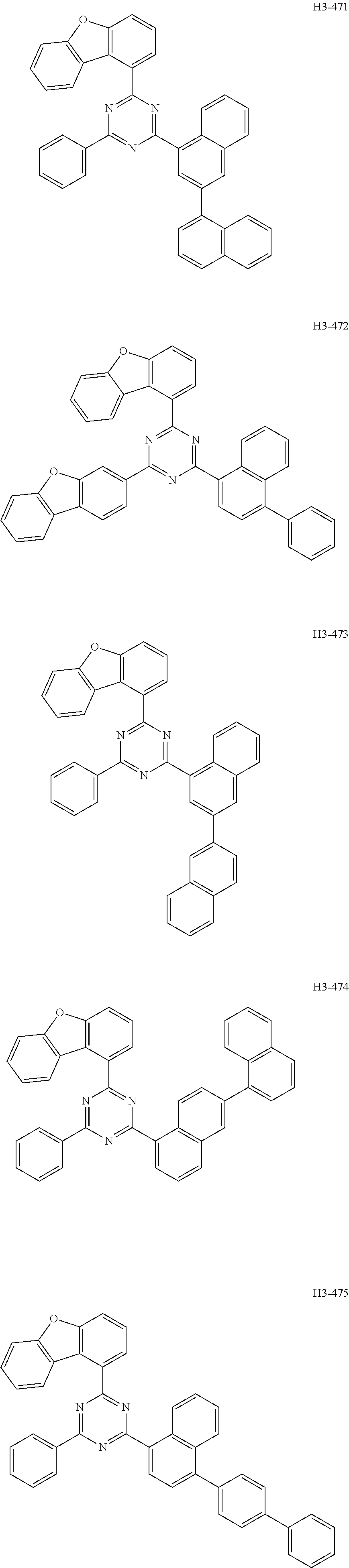 [SMILES: c1ccc(-c2nc(-c3cc(-c4cccc5ccccc45)cc4ccccc34)nc(-c3cccc4oc5ccccc5c34)n2)cc1 ; c1ccc(-c2ccc(-c3nc(-c4ccc5c(c4)oc4ccccc45)nc(-c4cccc5oc6ccccc6c45)n3)c3ccccc23)cc1 ; c1ccc(-c2nc(-c3cc(-c4ccc5ccccc5c4)cc4ccccc34)nc(-c3cccc4oc5ccccc5c34)n2)cc1 ; c1ccc(-c2nc(-c3cccc4cc(-c5cccc6ccccc56)ccc34)nc(-c3cccc4oc5ccccc5c34)n2)cc1 ; c1ccc(-c2ccc(-c3ccc(-c4nc(-c5ccccc5)nc(-c5cccc6oc7ccccc7c56)n4)c4ccccc34)cc2)cc1] 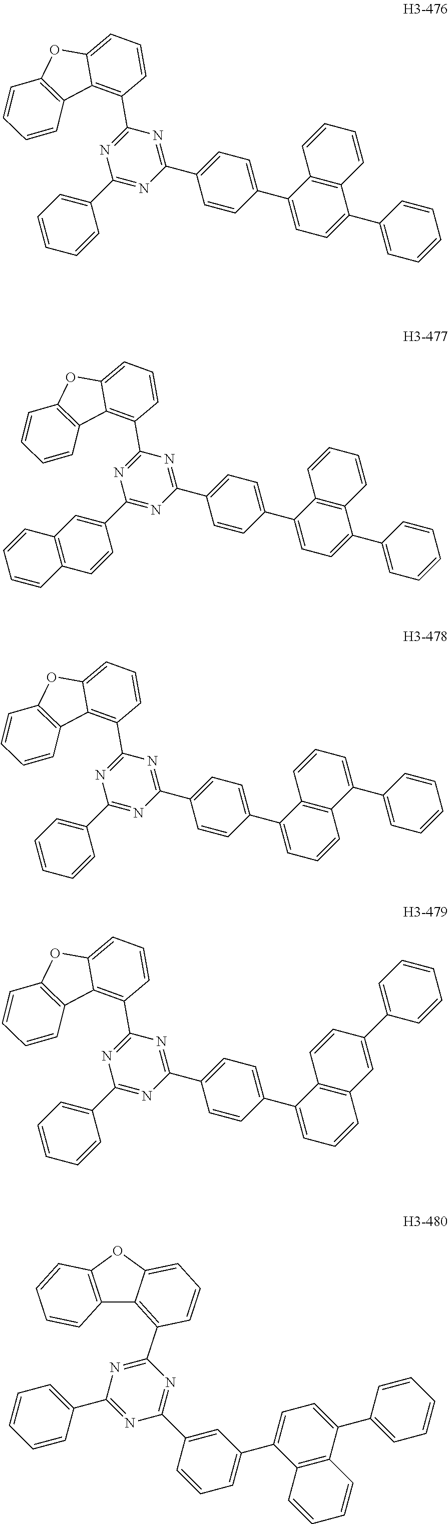 [SMILES: c1ccc(-c2nc(-c3ccc(-c4ccc(-c5ccccc5)c5ccccc45)cc3)nc(-c3cccc4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)c3ccccc23)cc1 ; c1ccc(-c2nc(-c3ccc(-c4cccc5c(-c6ccccc6)cccc45)cc3)nc(-c3cccc4oc5ccccc5c34)n2)cc1 ; c1ccc(-c2ccc3c(-c4ccc(-c5nc(-c6ccccc6)nc(-c6cccc7oc8ccccc8c67)n5)cc4)cccc3c2)cc1 ; c1ccc(-c2nc(-c3cccc(-c4ccc(-c5ccccc5)c5ccccc45)c3)nc(-c3cccc4oc5ccccc5c34)n2)cc1] 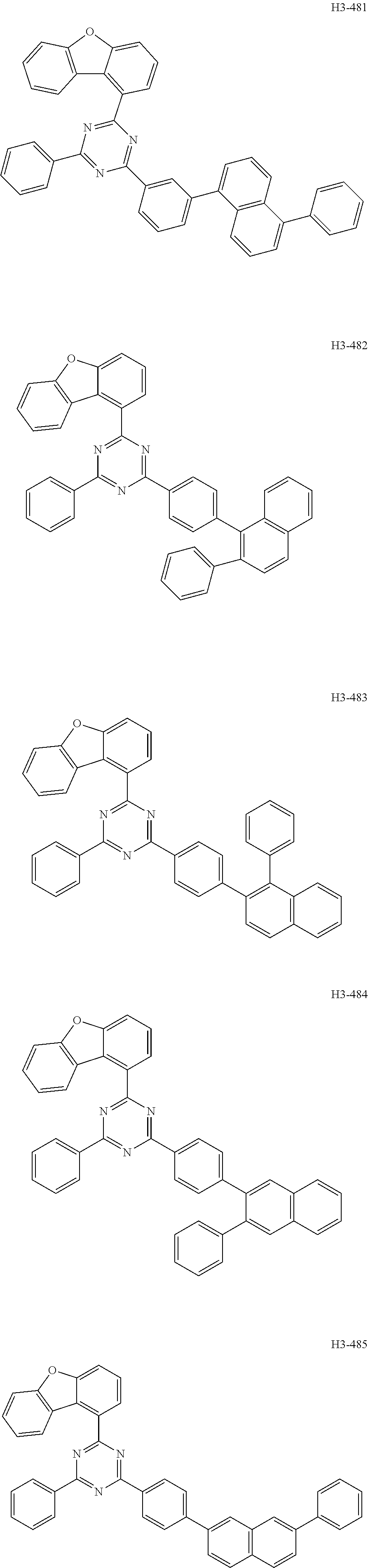 [SMILES: c1ccc(-c2nc(-c3cccc(-c4cccc5c(-c6ccccc6)cccc45)c3)nc(-c3cccc4oc5ccccc5c34)n2)cc1 ; c1ccc(-c2nc(-c3ccc(-c4c(-c5ccccc5)ccc5ccccc45)cc3)nc(-c3cccc4oc5ccccc5c34)n2)cc1 ; c1ccc(-c2nc(-c3ccc(-c4ccc5ccccc5c4-c4ccccc4)cc3)nc(-c3cccc4oc5ccccc5c34)n2)cc1 ; c1ccc(-c2nc(-c3ccc(-c4cc5ccccc5cc4-c4ccccc4)cc3)nc(-c3cccc4oc5ccccc5c34)n2)cc1 ; c1ccc(-c2ccc3ccc(-c4ccc(-c5nc(-c6ccccc6)nc(-c6cccc7oc8ccccc8c67)n5)cc4)cc3c2)cc1] 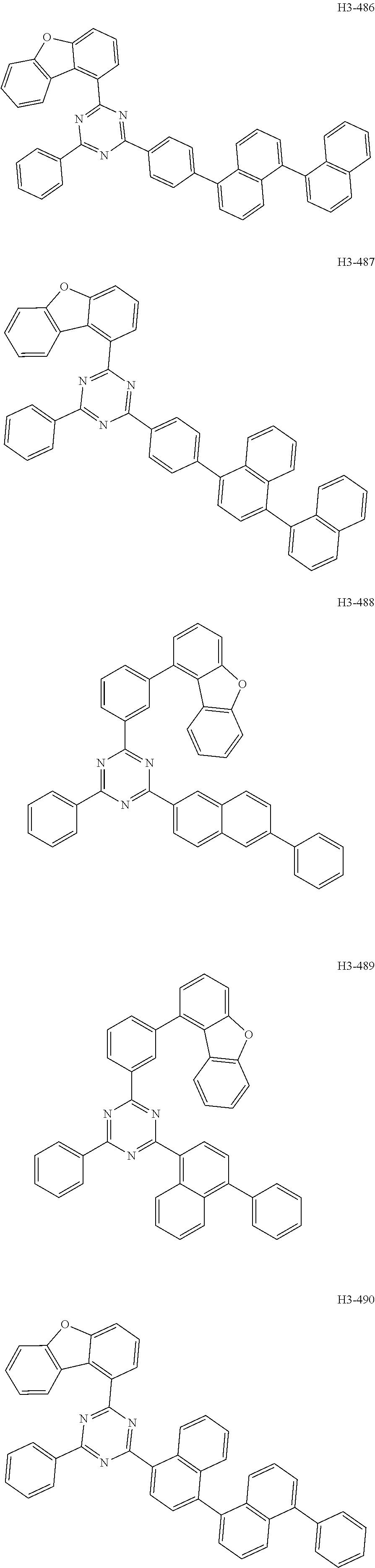 [SMILES: c1ccc(-c2nc(-c3ccc(-c4cccc5c(-c6cccc7ccccc67)cccc45)cc3)nc(-c3cccc4oc5ccccc5c34)n2)cc1 ; c1ccc(-c2nc(-c3ccc(-c4ccc(-c5cccc6ccccc56)c5ccccc45)cc3)nc(-c3cccc4oc5ccccc5c34)n2)cc1 ; c1ccc(-c2ccc3cc(-c4nc(-c5ccccc5)nc(-c5cccc(-c6cccc7oc8ccccc8c67)c5)n4)ccc3c2)cc1 ; c1ccc(-c2nc(-c3cccc(-c4cccc5oc6ccccc6c45)c3)nc(-c3ccc(-c4ccccc4)c4ccccc34)n2)cc1 ; c1ccc(-c2nc(-c3ccc(-c4cccc5c(-c6ccccc6)cccc45)c4ccccc34)nc(-c3cccc4oc5ccccc5c34)n2)cc1] 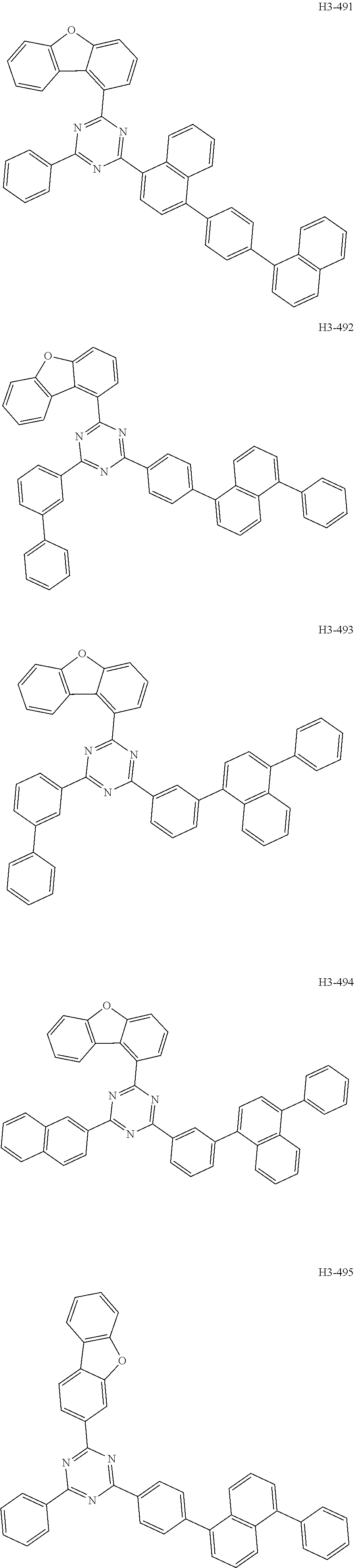 [SMILES: c1ccc(-c2nc(-c3ccc(-c4ccc(-c5cccc6ccccc56)cc4)c4ccccc34)nc(-c3cccc4oc5ccccc5c34)n2)cc1 ; c1ccc(-c2cccc(-c3nc(-c4ccc(-c5cccc6c(-c7ccccc7)cccc56)cc4)nc(-c4cccc5oc6ccccc6c45)n3)c2)cc1 ; c1ccc(-c2cccc(-c3nc(-c4cccc(-c5ccc(-c6ccccc6)c6ccccc56)c4)nc(-c4cccc5oc6ccccc6c45)n3)c2)cc1 ; c1ccc(-c2ccc(-c3cccc(-c4nc(-c5ccc6ccccc6c5)nc(-c5cccc6oc7ccccc7c56)n4)c3)c3ccccc23)cc1 ; c1ccc(-c2nc(-c3ccc(-c4cccc5c(-c6ccccc6)cccc45)cc3)nc(-c3ccc4c(c3)oc3ccccc34)n2)cc1] 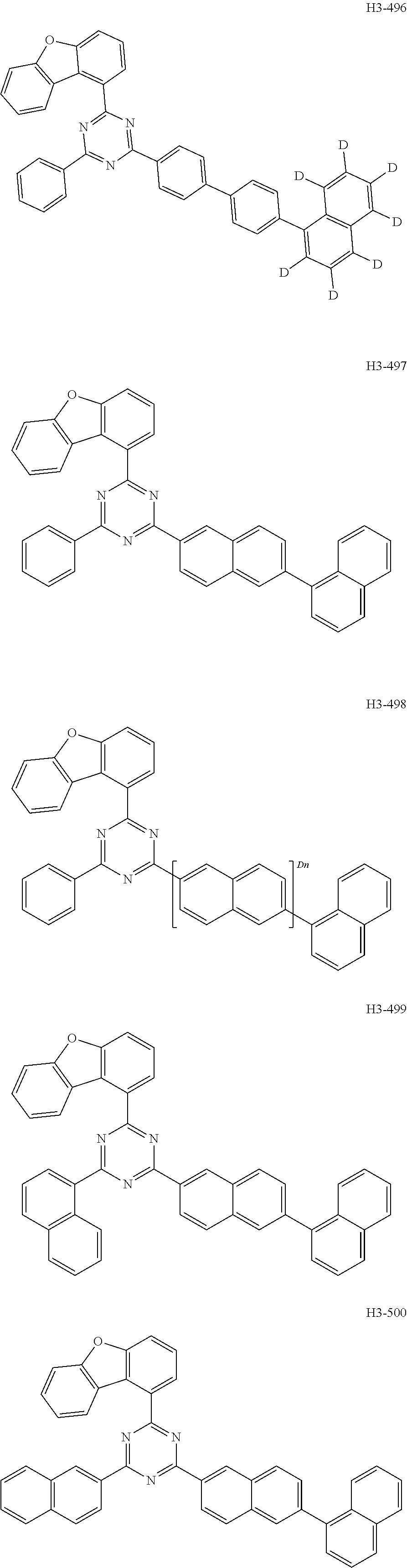 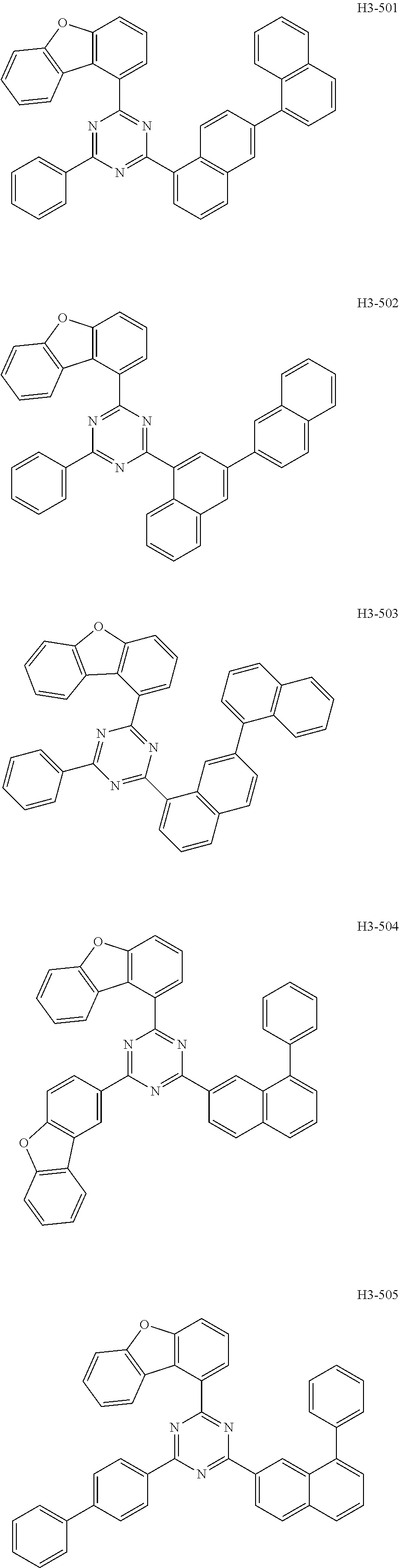 [SMILES: c1ccc(-c2nc(-c3cccc4cc(-c5cccc6ccccc56)ccc34)nc(-c3cccc4oc5ccccc5c34)n2)cc1 ; c1ccc(-c2nc(-c3cc(-c4ccc5ccccc5c4)cc4ccccc34)nc(-c3cccc4oc5ccccc5c34)n2)cc1 ; c1ccc(-c2nc(-c3cccc4ccc(-c5cccc6ccccc56)cc34)nc(-c3cccc4oc5ccccc5c34)n2)cc1 ; c1ccc(-c2cccc3ccc(-c4nc(-c5ccc6oc7ccccc7c6c5)nc(-c5cccc6oc7ccccc7c56)n4)cc23)cc1 ; c1ccc(-c2ccc(-c3nc(-c4ccc5cccc(-c6ccccc6)c5c4)nc(-c4cccc5oc6ccccc6c45)n3)cc2)cc1] 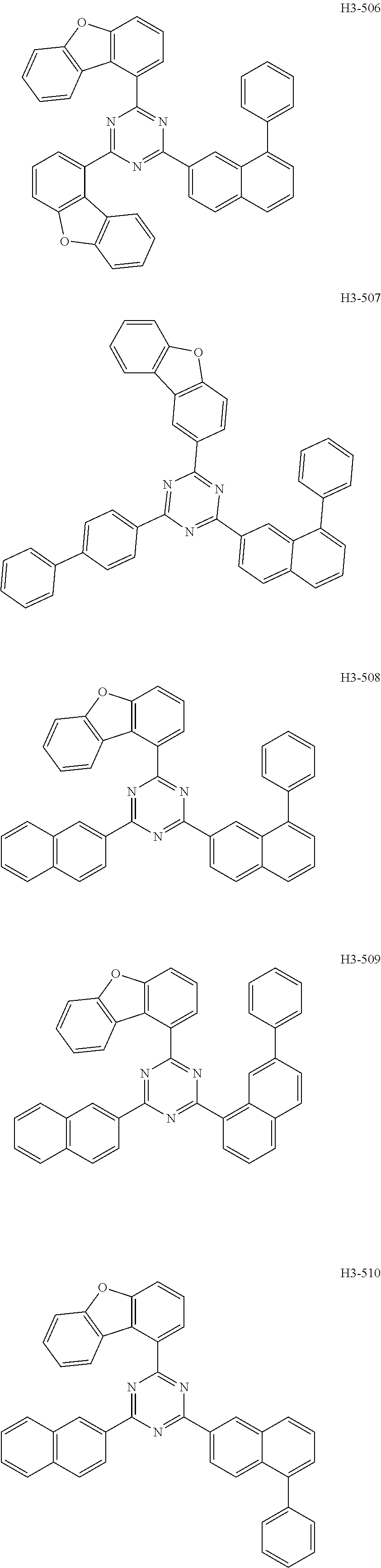 [SMILES: c1ccc(-c2cccc3ccc(-c4nc(-c5cccc6oc7ccccc7c56)nc(-c5cccc6oc7ccccc7c56)n4)cc23)cc1 ; c1ccc(-c2ccc(-c3nc(-c4ccc5cccc(-c6ccccc6)c5c4)nc(-c4ccc5oc6ccccc6c5c4)n3)cc2)cc1 ; c1ccc(-c2cccc3ccc(-c4nc(-c5ccc6ccccc6c5)nc(-c5cccc6oc7ccccc7c56)n4)cc23)cc1 ; c1ccc(-c2ccc3cccc(-c4nc(-c5ccc6ccccc6c5)nc(-c5cccc6oc7ccccc7c56)n4)c3c2)cc1 ; c1ccc(-c2cccc3cc(-c4nc(-c5ccc6ccccc6c5)nc(-c5cccc6oc7ccccc7c56)n4)ccc23)cc1] 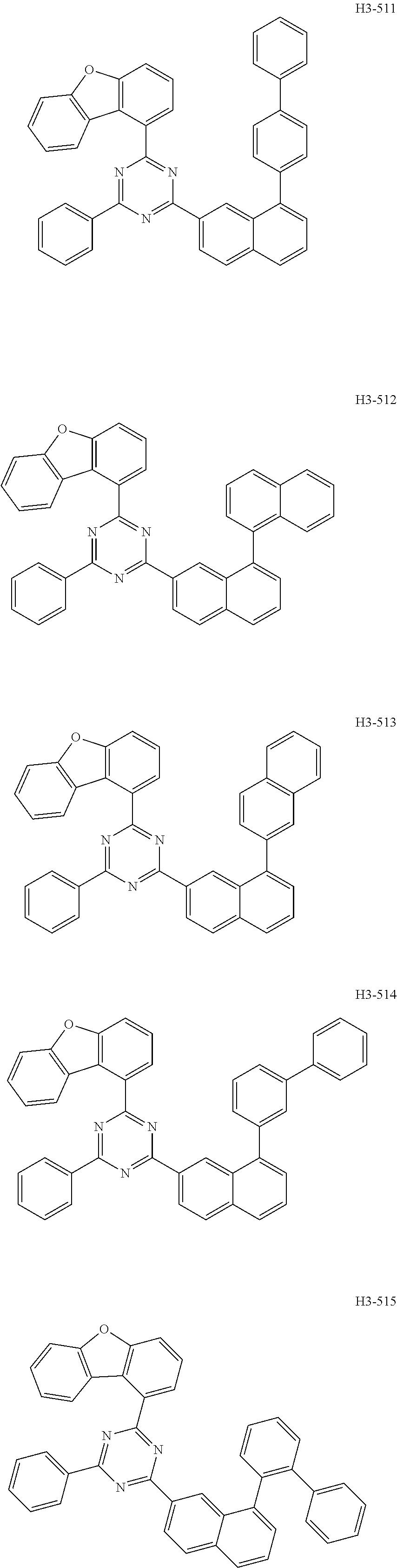 [SMILES: c1ccc(-c2ccc(-c3cccc4ccc(-c5nc(-c6ccccc6)nc(-c6cccc7oc8ccccc8c67)n5)cc34)cc2)cc1 ; c1ccc(-c2nc(-c3ccc4cccc(-c5cccc6ccccc56)c4c3)nc(-c3cccc4oc5ccccc5c34)n2)cc1 ; c1ccc(-c2nc(-c3ccc4cccc(-c5ccc6ccccc6c5)c4c3)nc(-c3cccc4oc5ccccc5c34)n2)cc1 ; c1ccc(-c2cccc(-c3cccc4ccc(-c5nc(-c6ccccc6)nc(-c6cccc7oc8ccccc8c67)n5)cc34)c2)cc1 ; c1ccc(-c2nc(-c3ccc4cccc(-c5ccccc5-c5ccccc5)c4c3)nc(-c3cccc4oc5ccccc5c34)n2)cc1] 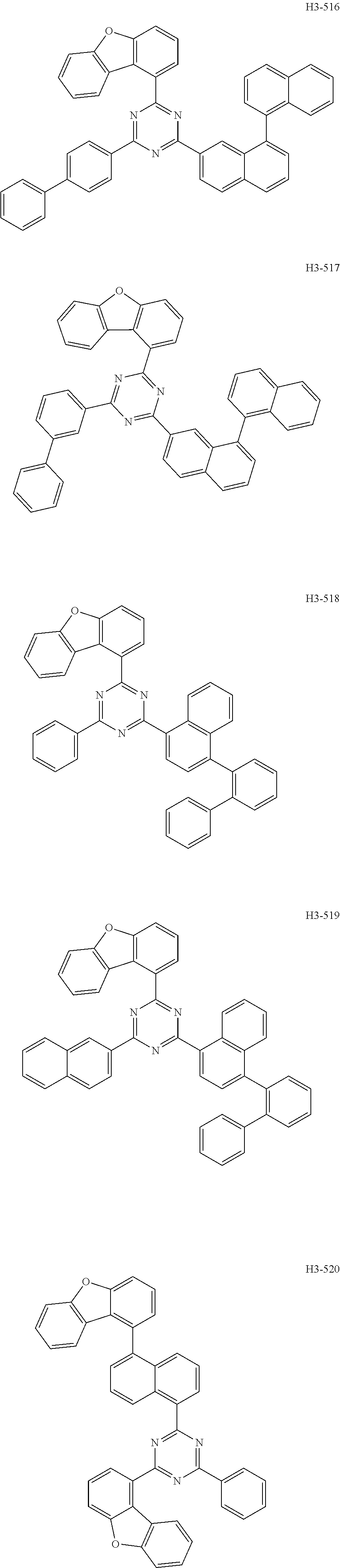 [SMILES: c1ccc(-c2ccc(-c3nc(-c4ccc5cccc(-c6cccc7ccccc67)c5c4)nc(-c4cccc5oc6ccccc6c45)n3)cc2)cc1 ; c1ccc(-c2cccc(-c3nc(-c4ccc5cccc(-c6cccc7ccccc67)c5c4)nc(-c4cccc5oc6ccccc6c45)n3)c2)cc1 ; c1ccc(-c2nc(-c3ccc(-c4ccccc4-c4ccccc4)c4ccccc34)nc(-c3cccc4oc5ccccc5c34)n2)cc1 ; c1ccc(-c2ccccc2-c2ccc(-c3nc(-c4ccc5ccccc5c4)nc(-c4cccc5oc6ccccc6c45)n3)c3ccccc23)cc1 ; c1ccc(-c2nc(-c3cccc4c(-c5cccc6oc7ccccc7c56)cccc34)nc(-c3cccc4oc5ccccc5c34)n2)cc1] 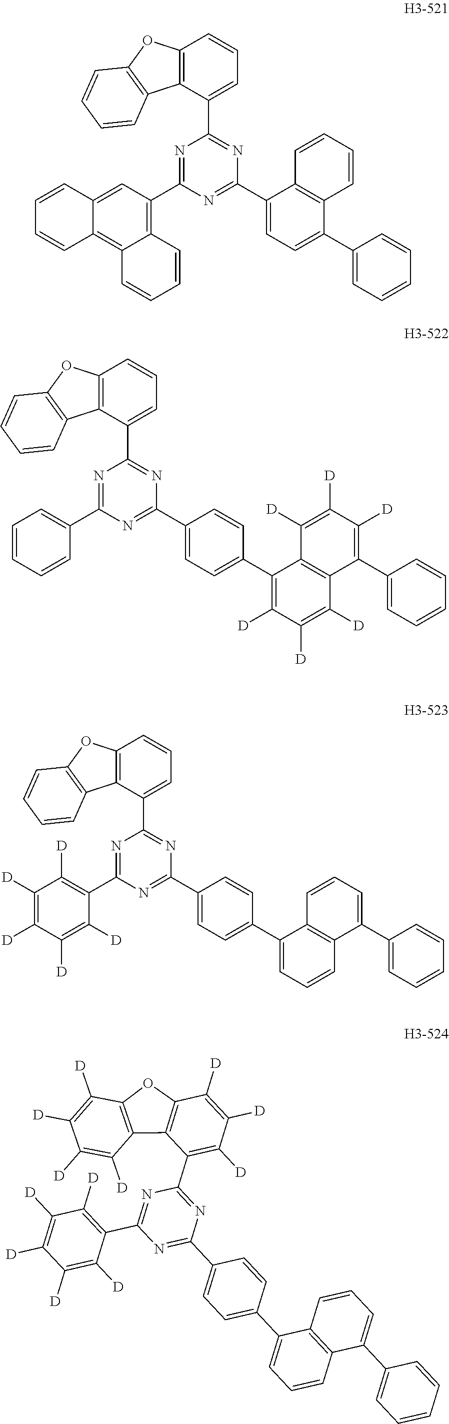 [SMILES: c1ccc(-c2ccc(-c3nc(-c4cc5ccccc5c5ccccc45)nc(-c4cccc5oc6ccccc6c45)n3)c3ccccc23)cc1 ; [2H]c1c([2H])c(-c2ccc(-c3nc(-c4ccccc4)nc(-c4cccc5oc6ccccc6c45)n3)cc2)c2c([2H])c([2H])c([2H])c(-c3ccccc3)c2c1[2H] ; [2H]c1c([2H])c([2H])c(-c2nc(-c3ccc(-c4cccc5c(-c6ccccc6)cccc45)cc3)nc(-c3cccc4oc5ccccc5c34)n2)c([2H])c1[2H] ; [2H]c1c([2H])c([2H])c(-c2nc(-c3ccc(-c4cccc5c(-c6ccccc6)cccc45)cc3)nc(-c3c([2H])c([2H])c([2H])c4oc5c([2H])c([2H])c([2H])c([2H])c5c34)n2)c([2H])c1[2H]] 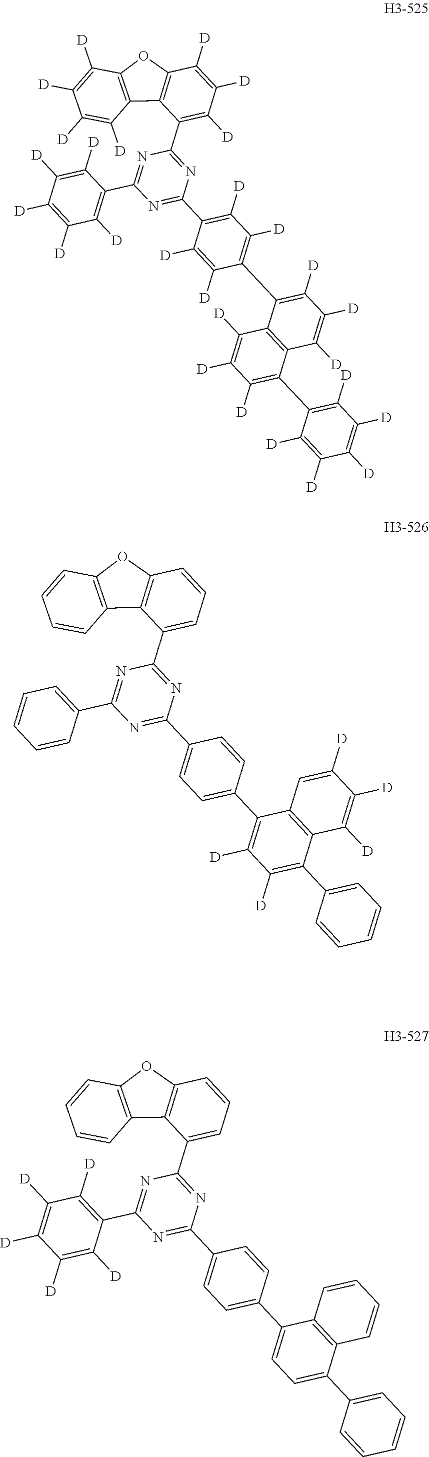 [SMILES: [2H]c1c([2H])c([2H])c(-c2nc(-c3c([2H])c([2H])c(-c4c([2H])c([2H])c([2H])c5c(-c6c([2H])c([2H])c([2H])c([2H])c6[2H])c([2H])c([2H])c([2H])c45)c([2H])c3[2H])nc(-c3c([2H])c([2H])c([2H])c4oc5c([2H])c([2H])c([2H])c([2H])c5c34)n2)c([2H])c1[2H] ; [2H]c1cc2c(-c3ccc(-c4nc(-c5ccccc5)nc(-c5cccc6oc7ccccc7c56)n4)cc3)c([2H])c([2H])c(-c3ccccc3)c2c([2H])c1[2H] ; [2H]c1c([2H])c([2H])c(-c2nc(-c3ccc(-c4ccc(-c5ccccc5)c5ccccc45)cc3)nc(-c3cccc4oc5ccccc5c34)n2)c([2H])c1[2H]] 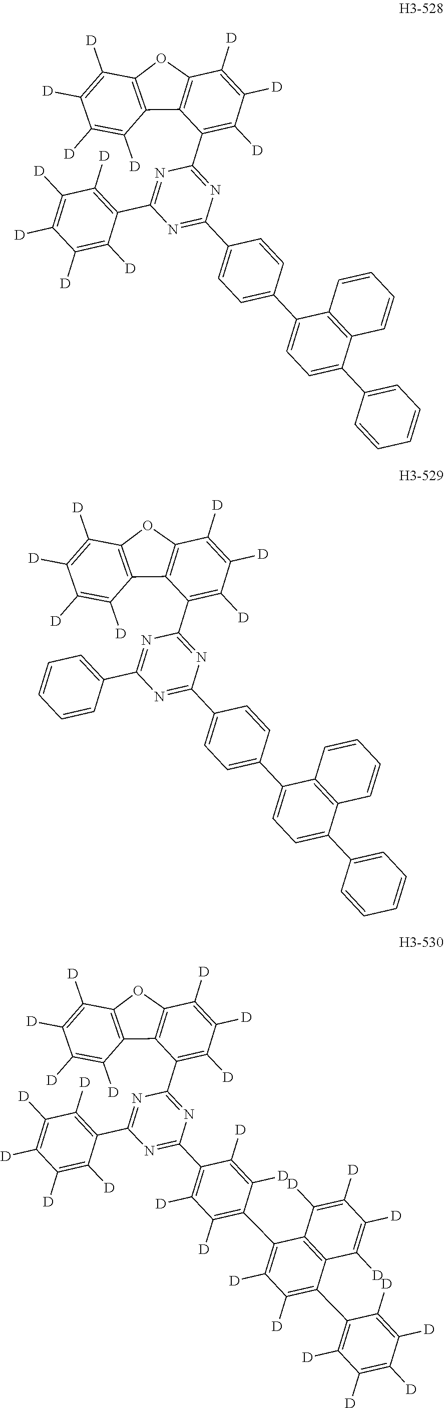 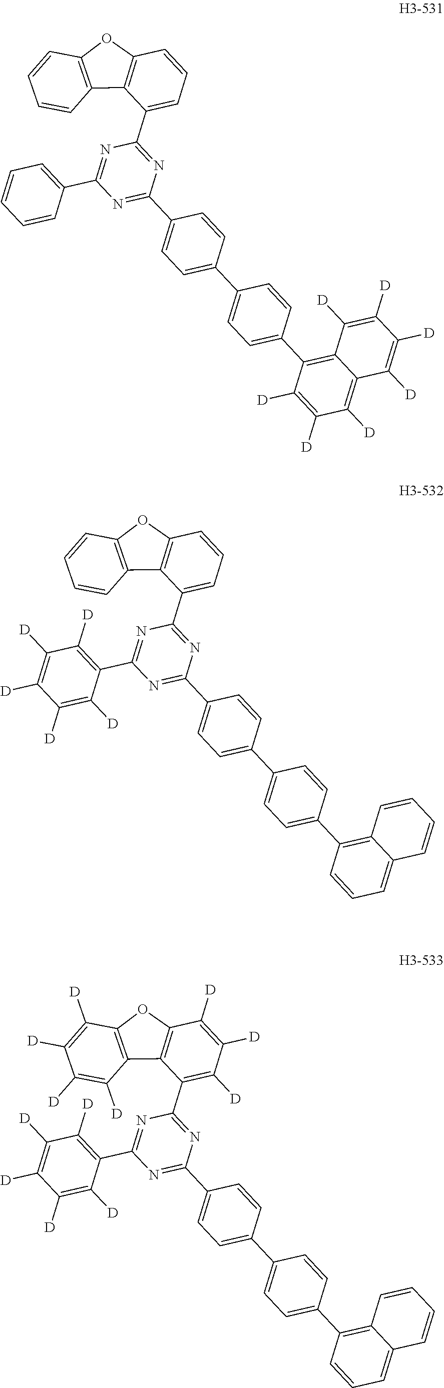 [SMILES: [2H]c1c([2H])c([2H])c2c(-c3ccc(-c4ccc(-c5nc(-c6ccccc6)nc(-c6cccc7oc8ccccc8c67)n5)cc4)cc3)c([2H])c([2H])c([2H])c2c1[2H] ; [2H]c1c([2H])c([2H])c(-c2nc(-c3ccc(-c4ccc(-c5cccc6ccccc56)cc4)cc3)nc(-c3cccc4oc5ccccc5c34)n2)c([2H])c1[2H] ; [2H]c1c([2H])c([2H])c(-c2nc(-c3ccc(-c4ccc(-c5cccc6ccccc56)cc4)cc3)nc(-c3c([2H])c([2H])c([2H])c4oc5c([2H])c([2H])c([2H])c([2H])c5c34)n2)c([2H])c1[2H]] 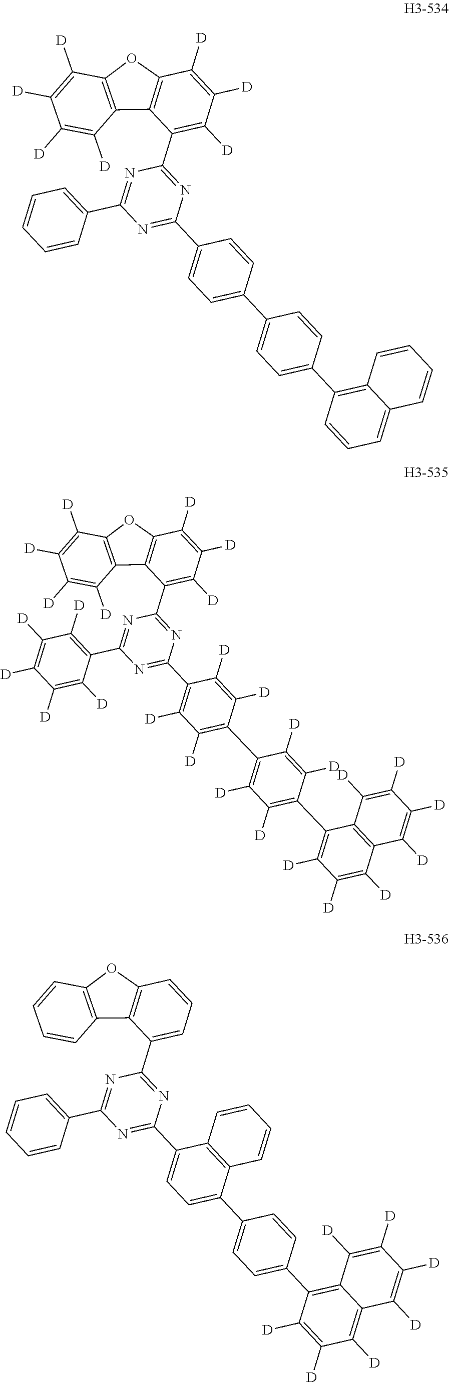 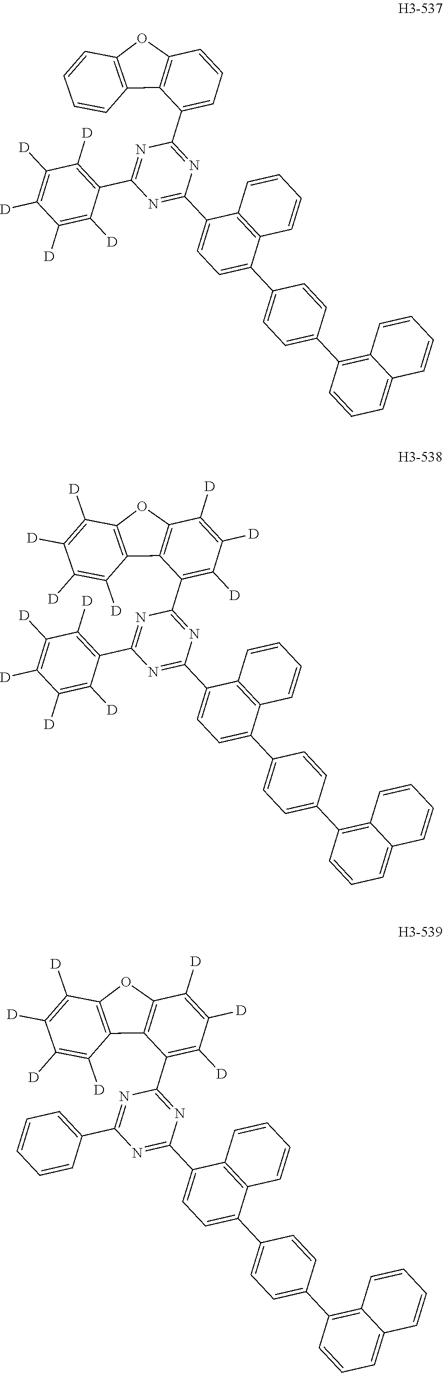 [SMILES: [2H]c1c([2H])c([2H])c(-c2nc(-c3ccc(-c4ccc(-c5cccc6ccccc56)cc4)c4ccccc34)nc(-c3cccc4oc5ccccc5c34)n2)c([2H])c1[2H] ; [2H]c1c([2H])c([2H])c(-c2nc(-c3ccc(-c4ccc(-c5cccc6ccccc56)cc4)c4ccccc34)nc(-c3c([2H])c([2H])c([2H])c4oc5c([2H])c([2H])c([2H])c([2H])c5c34)n2)c([2H])c1[2H] ; [2H]c1c([2H])c([2H])c2c(oc3c([2H])c([2H])c([2H])c(-c4nc(-c5ccccc5)nc(-c5ccc(-c6ccc(-c7cccc8ccccc78)cc6)c6ccccc56)n4)c32)c1[2H]] 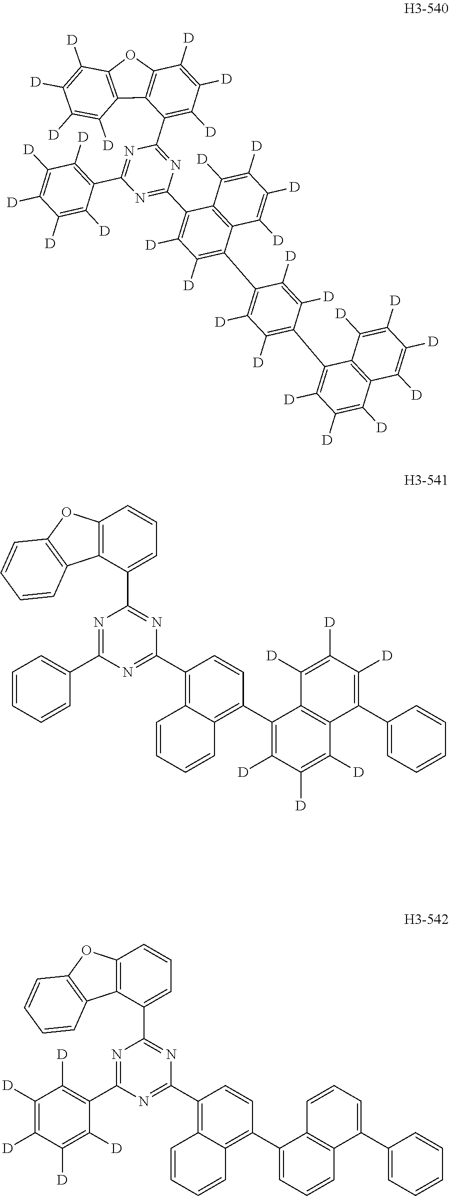 [SMILES: [2H]c1c([2H])c([2H])c(-c2nc(-c3c([2H])c([2H])c(-c4c([2H])c([2H])c(-c5c([2H])c([2H])c([2H])c6c([2H])c([2H])c([2H])c([2H])c56)c([2H])c4[2H])c4c([2H])c([2H])c([2H])c([2H])c34)nc(-c3c([2H])c([2H])c([2H])c4oc5c([2H])c([2H])c([2H])c([2H])c5c34)n2)c([2H])c1[2H] ; [2H]c1c([2H])c(-c2ccc(-c3nc(-c4ccccc4)nc(-c4cccc5oc6ccccc6c45)n3)c3ccccc23)c2c([2H])c([2H])c([2H])c(-c3ccccc3)c2c1[2H] ; [2H]c1c([2H])c([2H])c(-c2nc(-c3ccc(-c4cccc5c(-c6ccccc6)cccc45)c4ccccc34)nc(-c3cccc4oc5ccccc5c34)n2)c([2H])c1[2H]] 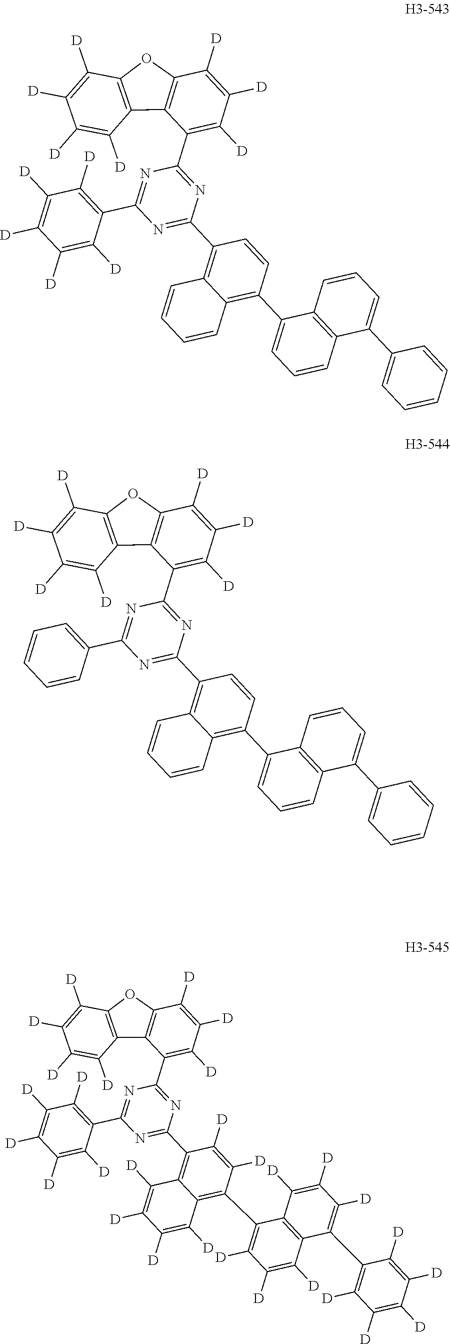 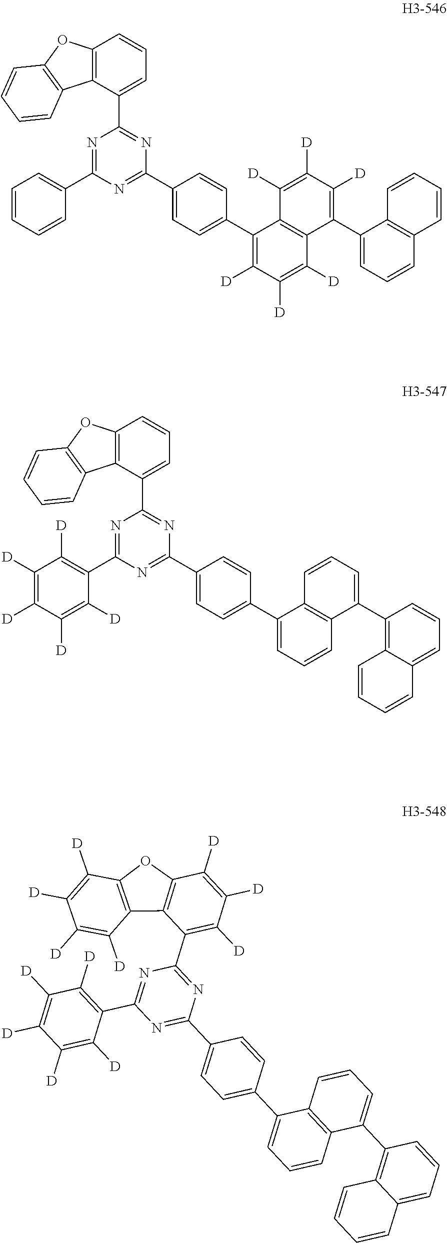 [SMILES: [2H]c1c([2H])c(-c2cccc3ccccc23)c2c([2H])c([2H])c([2H])c(-c3ccc(-c4nc(-c5ccccc5)nc(-c5cccc6oc7ccccc7c56)n4)cc3)c2c1[2H] ; [2H]c1c([2H])c([2H])c(-c2nc(-c3ccc(-c4cccc5c(-c6cccc7ccccc67)cccc45)cc3)nc(-c3cccc4oc5ccccc5c34)n2)c([2H])c1[2H] ; [2H]c1c([2H])c([2H])c(-c2nc(-c3ccc(-c4cccc5c(-c6cccc7ccccc67)cccc45)cc3)nc(-c3c([2H])c([2H])c([2H])c4oc5c([2H])c([2H])c([2H])c([2H])c5c34)n2)c([2H])c1[2H]] 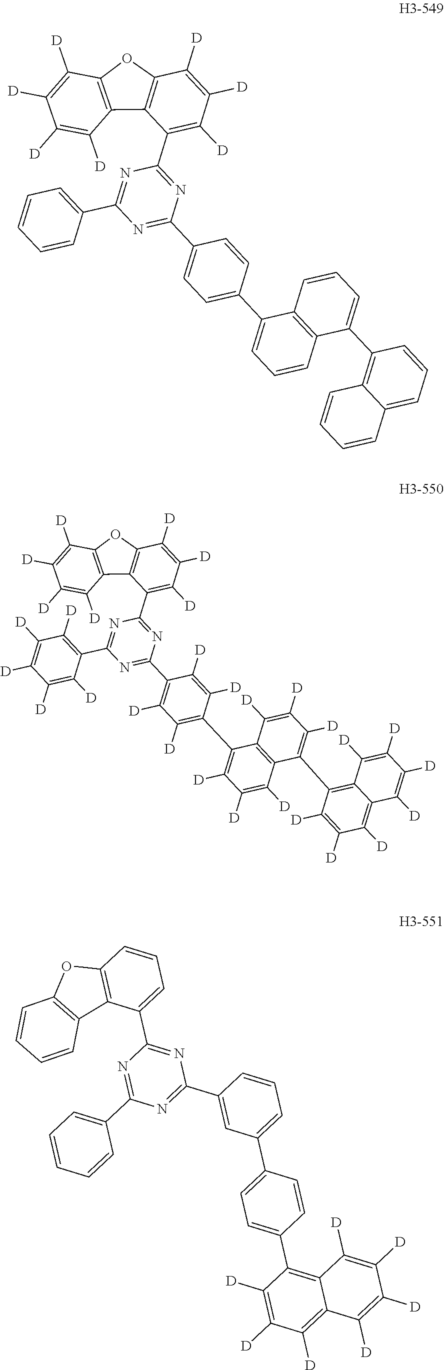 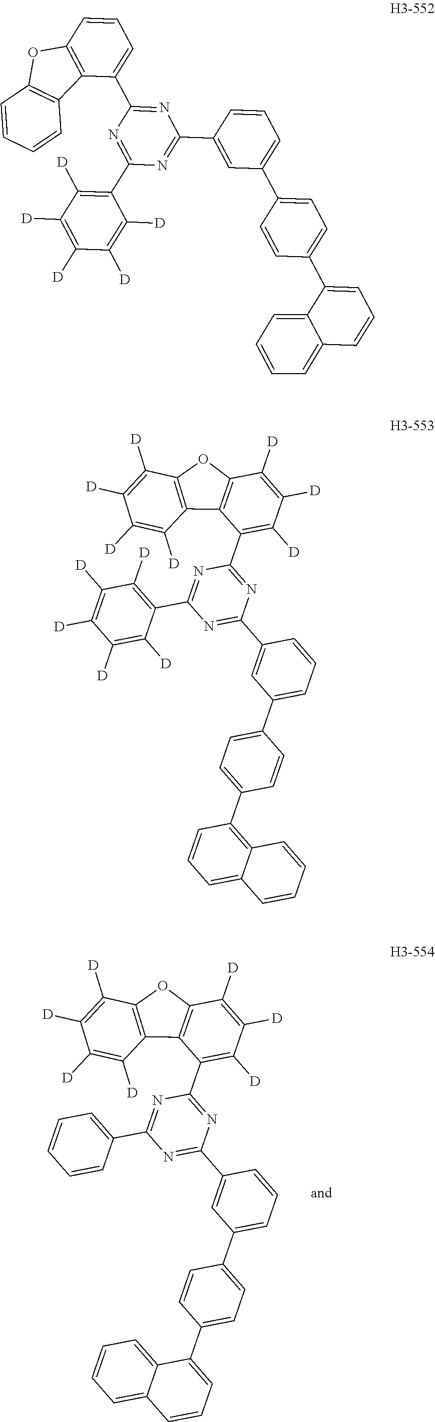 [SMILES: [2H]c1c([2H])c([2H])c(-c2nc(-c3cccc(-c4ccc(-c5cccc6ccccc56)cc4)c3)nc(-c3cccc4oc5ccccc5c34)n2)c([2H])c1[2H] ; [2H]c1c([2H])c([2H])c(-c2nc(-c3cccc(-c4ccc(-c5cccc6ccccc56)cc4)c3)nc(-c3c([2H])c([2H])c([2H])c4oc5c([2H])c([2H])c([2H])c([2H])c5c34)n2)c([2H])c1[2H] ; [2H]c1c([2H])c([2H])c2c(oc3c([2H])c([2H])c([2H])c(-c4nc(-c5ccccc5)nc(-c5cccc(-c6ccc(-c7cccc8ccccc78)cc6)c5)n4)c32)c1[2H]] 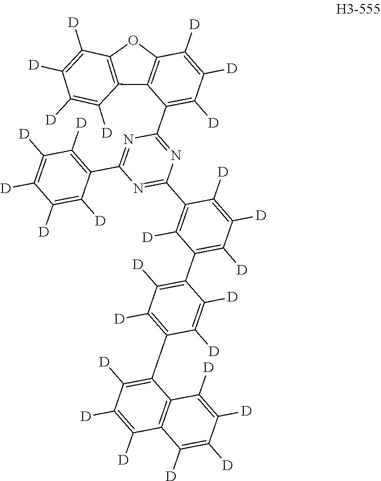 [SMILES: [2H]c1c([2H])c([2H])c(-c2nc(-c3c([2H])c([2H])c([2H])c(-c4c([2H])c([2H])c(-c5c([2H])c([2H])c([2H])c6c([2H])c([2H])c([2H])c([2H])c56)c([2H])c4[2H])c3[2H])nc(-c3c([2H])c([2H])c([2H])c4oc5c([2H])c([2H])c([2H])c([2H])c5c34)n2)c([2H])c1[2H]]
wherein in the compounds above, Dₙ means that n number of hydrogens are replaced with deuterium, and n represents an integer from 1 to the maximum number of hydrogens in the compound.

18. The organic electroluminescent device according to claim 12, wherein the light-emitting layer further comprises an additional compound different from the compounds represented by Formula 6 and Formula 7.
